(19) United States
(12) Patent Application Publication
(10) Pub. No.: US 2026/0337319 A1
(43) Pub. Date: Oct. 29, 2026

(54) SULFONIUM SALT MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/632,626

(22) Filed: Mar. 30, 2026

(30) Foreign Application Priority Data
Apr. 4, 2025 (JP) 2025-062615

(51) Int. Cl. C08F 220/22; C08F 212/14; C08F 212/32; C08F 220/18; C08F 220/30; G03F 7/004; G03F 7/039; G03F 7/20

(52) U.S. Cl. CPC C08F 220/22; C08F 212/24; C08F 212/32; C08F 220/1807; C08F 220/301; G03F 7/0045; G03F 7/0397; G03F 7/2004

(57) ABSTRACT

Provided are a sulfonium salt monomer, a polymer comprising repeat units derived from the sulfonium salt monomer, a chemically amplified resist composition comprising the polymer, and a pattern forming process using the chemically amplified resist composition, the sulfonium salt monomer being used in a chemically amplified resist composition that exhibits a satisfactory solvent solubility, high sensitivity, high contrast, and improved lithography properties including LWR, CDU, EL and DOF when processed by photolithography using high-energy radiation. The sulfonium salt monomer has the formula (A), wherein Z⁻ is a fluoroalkanesulfonate anion having a polymerizable group.

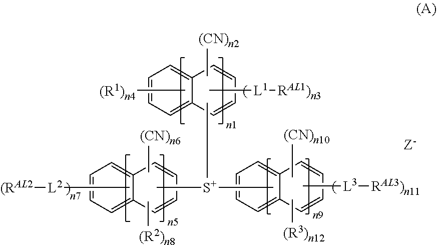

### CROSS-REFERENCE TO RELATED APPLICATION

This non-provisional application claims priority under 35 U.S.C. § 119(a) on Patent Application No. 2025-062615 filed in Japan on Apr. 4, 2025, the entire contents of which are hereby incorporated by reference.

### TECHNICAL FIELD

This invention relates to a sulfonium salt monomer, a polymer, a chemically amplified resist composition, and a pattern forming process.

### BACKGROUND ART

To meet the demand for higher integration density and operating speed of LSIs, the effort to reduce the pattern rule is in rapid progress. In particular, the expansion of the flash memory market and the increase in storage capacity are driving miniaturization. As state-of-the-art miniaturization technology, mass production of 65-nm node devices by ArF lithography has been carried out, and preparations for mass production of 45-nm node devices by next-generation ArF immersion lithography are in progress. Candidates for next-generation 32-nm node devices include immersion lithography using an ultra-high-NA lens that combines a liquid having a refractive index higher than that of water, a high-refractive-index lens, and a high-refractive-index resist film; extreme ultraviolet (EUV) lithography at a wavelength of 13.5 nm; and ArF lithography using double exposure (double patterning), all of which are under active consideration.

As miniaturization progresses and approaches the diffraction limit of light, optical contrast decreases. As a result of the reduction in optical contrast, a positive resist film exhibits reduced resolution and a reduced focus margin for hole and trench patterns.

With progressive pattern miniaturization, line width roughness (LWR) of line patterns and critical dimension uniformity (CDU) of hole patterns have become issues of concern. These issues have been attributed to non-uniform distribution or agglomeration of a base polymer and an acid generator, as well as to acid diffusion. Furthermore, LWR tends to increase as the resist film thickness is reduced, and degradation of LWR caused by resist film thinning accompanying further miniaturization has become a serious problem.

In an EUV lithography resist composition, it is required to achieve high sensitivity, high resolution, and low LWR simultaneously. When the acid diffusion distance is shortened, LWR is reduced, but sensitivity is also reduced. For example, lowering the post-exposure bake (PEB) temperature reduces LWR but also lowers sensitivity. Increasing the amount of quencher added likewise reduces LWR but also lowers sensitivity. Accordingly, it is necessary to overcome the trade-off relationship between sensitivity and LWR.

With the aim to suppress acid diffusion, Patent Document 1 discloses a resist compound comprising repeat units derived from an onium salt of a polymerizable unsaturated bond-containing sulfonic acid. Since the so-called polymer-bound acid generator is capable of generating a polymeric sulfonic acid upon exposure, it is characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, an increase of acid diffusion leads to degraded LWR or CDU. With respect to a balance of sensitivity, LWR and CDU, the polymer-bound acid generator is regarded as having a high capability.

Since iodine atoms are highly absorptive to EUV of wavelength 13.5 nm, they generate secondary electrons upon light exposure. This effect is noteworthy in the EUV lithography. Patent Document 2 describes a photoacid generator having an iodized anion. Patent Documents 3 and 4 describe a photoacid generator having an iodized anion and containing a polymerizable group. Although the lithography performance is improved to some extent, the organic solvent solubility of iodine-containing compounds is not so high, accompanied with a concern about precipitation in the solvent.

Patent Documents 5 and 6 describe photoacid generators in which a pentafluorosulfanyl group (—SF₅ group) is introduced into a cation. Patent Document 7 describes a photoacid generator in which a trifluoromethoxy group (—OCF₃ group) is introduced. Patent Document 8 describes a photoacid generator containing a sulfonium cation having a fluorine atom or a trifluoromethoxy group, and an acid labile group. Patent Document 9 describes a photoacid generator containing a sulfonium cation into which an iodine atom and an acid labile group are introduced. Although lithographic performance has been improved to some extent by these approaches, room for improvement still remains, and development of resist materials effective for further fine pattern formation is desired.

### CITATION LIST

Patent Document 1: JP 4425776
Patent Document 2: JP 6720926
Patent Document 3: JP 6973274
Patent Document 4: JP-A 2024-043941
Patent Document 5: WO 2023/223624
Patent Document 6: WO 2024/128017
Patent Document 7: JP-A 2022-059112
Patent Document 8: JP-A 2023-169812
Patent Document 9: JP 7367185

### SUMMARY OF THE INVENTION

In acid-catalyzed chemically amplified resist compositions, development of compositions capable of achieving further increased sensitivity while improving lithographic performance, including LWR, CDU, exposure latitude (EL), and depth of focus (DOF), is desired.

An object of the invention is to provide a sulfonium salt monomer, a polymer comprising repeat units derived from the monomer, and a chemically amplified resist composition comprising the polymer, the chemically amplified resist composition, when processed by photolithography using high-energy radiation such as KrF excimer laser, ArF excimer laser, EB or EUV, exhibiting a satisfactory solvent solubility, high sensitivity, high contrast, and improved lithography properties including LWR, CDU, EL, and DOF. Another object of the invention is to provide a pattern forming process using the chemically amplified resist composition.

The inventor has found that by using, as a polymer-bound acid generator, a polymer comprising repeat units derived from a sulfonium salt monomer containing a triarylsulfonium cation having, on the same aromatic ring, a cyano group and an acid labile group having a tertiary/secondary ether structure, a tertiary/secondary ester structure, a tertiary/secondary carbonate structure or an acetal structure, and a fluorosulfonate anion having a polymerizable group, it is possible to obtain a chemically amplified resist composition that exhibits high sensitivity, controlled acid diffusion of generated acid, improved lithographic performance such as LWR, CDU, EL, and DOF, high contrast, and high resolution.

That is, the invention provides a sulfonium salt monomer, a polymer, a chemically amplified resist composition, and a pattern forming process.

In one aspect, the invention provides a sulfonium salt monomer having the formula (A):

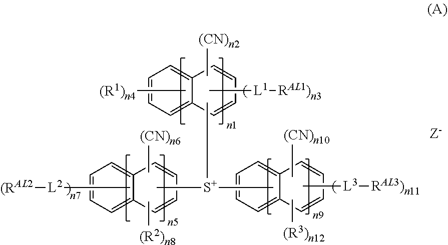

wherein n1 is 0 or 1, n2 is 0, 1 or 2, n3 is 0, 1 or 2, n4 is 0, 1 or 2, provided that 0≤n2+n3+n4≤5 when n1=0, and 0≤n2+n3+n4≤7 when n1=1; n5 is 0 or 1, n6 is 0, 1 or 2, n7 is 0, 1 or 2, n8 is 0, 1 or 2, provided that 0≤n6+n7+n8≤5 when n5=0, and 0≤n6+n7+n8≤7 when n5=1; n9 is 0 or 1, n10 is 0, 1 or 2, n11 is 0, 1 or 2, n12 is 0, 1 or 2, provided that 0≤n10+n11+n12≤5 when n9=0, and 0≤n10+n11+n12≤7 when n9=1, and 1≤n2+n6+n10≤6 and 1≤n3+n7+n11≤6,
R¹, R² and R³ are each independently halogen, nitro, hydroxy, carboxy, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom; when n4=2, two R¹ may be identical or different and may bond together to form a ring with the carbon atoms to which they are attached; when n8=2, two R² may be identical or different and may bond together to form a ring with the carbon atoms to which they are attached; when n12=2, two R³ may be identical or different and may bond together to form a ring with the carbon atoms to which they are attached,
L¹, L² and L³ are each independently an ether bond, ester bond or carbonate bond, two L¹ may be identical or different when n3=2, two L² may be identical or different when n7=2, two L³ may be identical or different when n11=2,
R^{AL1}, R^{AL2} and R^{AL3} are each independently an acid labile group, two R^{AL1} may be identical or different when n3=2, two R^{AL2} may be identical or different when n7=2, two R^{AL3} may be identical or different when n11=2,
two of three aromatic rings bonded to S⁺ may bond together to form a ring with the sulfur atom to which they are attached, and
Z⁻ is a fluoroalkanesulfonate anion having a polymerizable group.

The preferred sulfonium salt monomer has the formula (A1):

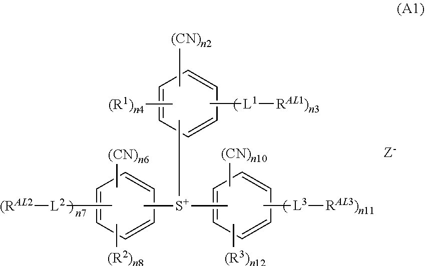

wherein n2 to n4, n6 to n8, n10 to n12, R¹ to R³, L¹ to L³, R^{AL1} to R^{AL3}, and Z⁻ are as defined above.

In a preferred embodiment, the acid labile group has the formula (AL-1) or (AL-2):

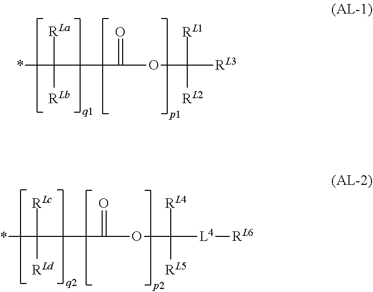

wherein p1 and p2 are each independently 0 or 1, q1 and q2 are each independently 0, 1, 2, 3 or 4,
R^{L1} and R^{L2} are each independently a C₁-C₁₂ hydrocarbyl group, R^{L3} is hydrogen or a C₁-C₁₂ hydrocarbyl group, some constituent —CH₂— in the hydrocarbyl groups R^{L1}, R^{L2} and R^{L3} may be replaced by —O— or —S—, and when hydrocarbyl group contains an aromatic ring, some or all of the hydrogen atoms on the aromatic ring may be substituted by halogen, cyano, nitro, a C₁-C₄ alkyl group which may contain a halogen atom or C₁-C₄ alkoxy group which may contain a halogen atom, R^{L1} and R^{L2} may bond together to form a ring with the carbon atom to which they are attached, and some constituent —CH₂— in the ring may be replaced by —O— or —S—, provided that when R^{L3} is hydrogen, R^{L1} and R^{L2} bond together to form an alicyclic ring containing a multiple bond together with the carbon atom to which they are bonded, some of the hydrogen atoms on the alicyclic ring may be replaced by halogen,
R^{L4} and R^{L5} are each independently hydrogen or a C₁-C₁₀ hydrocarbyl group, R^{L6} is a C₁-C₂₀ hydrocarbyl group in which some constituent —CH₂— may be replaced by —O— or —S—, R^{L5} and R^{L6} may bond together to form a C₃-C₂₀ heterocyclic group with the carbon atom and L⁴ to which they are attached, and some constituent —CH₂— in the heterocyclic group may be replaced by —O— or —S—,
L⁴ is —O— or —S—,
R^{La} to R^{Ld} are each independently hydrogen or a C₁-C₂₀ hydrocarbyl group which may contain a heteroatom, and
* designates a point of attachment to L¹, L² and L³.

In another preferred embodiment, Z is an anion having the formula (Z1):

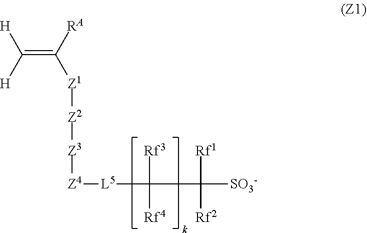

wherein k is 0, 1, 2 or 3,
R^{A} is each independently hydrogen, fluorine, methyl or trifluoromethyl,
Z¹ is each independently a single bond, optionally substituted phenylene group, naphthylene group or *—C(═O)—O—Z¹¹—, Z¹¹ is a C₁-C₁₀ aliphatic hydrocarbylene group which may contain halogen, hydroxy, ether bond, ester bond or lactone ring, a phenylene group or naphthylene group,
Z² is a single bond, ether bond, ester bond, amide bond, sulfonate ester bond, sulfonamide bond, carbonate bond or carbamate bond,
Z³ is each independently a single bond, ***—Z³¹—C(═O)—O—, ***—C(═O)—N(H)—Z³¹— or ***—O—Z³¹—, Z³¹ is a C₁-C₂₀ hydrocarbylene group which may contain a heteroatom,
Z⁴ is each independently a single bond, ****—Z⁴¹—C(═O)—O—, ****—C(═O)—N(H)—Z⁴¹— or ****—O—Z⁴¹—, Z⁴¹ is a C₁-C₂₀ hydrocarbylene group which may contain a heteroatom,
* designates a point of attachment to the carbon atom in the backbone, *** designates a point of attachment to Z², **** designates a point of attachment to Z³,
L⁵ is a single bond, ether bond, ester bond, carbonyl group, sulfonate ester bond, sulfonamide bond, carbonate bond or carbamate bond,
Rf¹ and Rf² are each independently fluorine or a C₁-C₆ fluorinated saturated hydrocarbyl group, and
Rf³ and Rf⁴ are each independently hydrogen, fluorine or a C₁-C₆ fluorinated saturated hydrocarbyl group.

In still another preferred embodiment, Z⁻ is an anion having the formula (Z2):

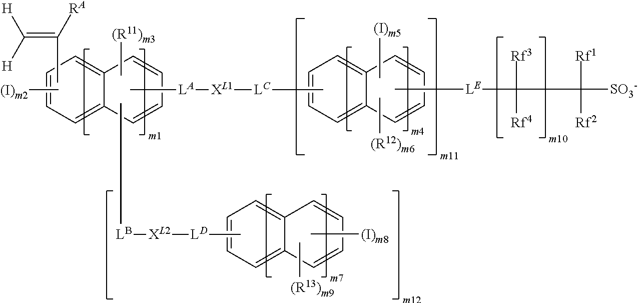

wherein m1 is 0 or 1, m2 is 0, 1, 2, 3 or 4, m3 is 0, 1, 2 or 3, m4 is 0 or 1, m5 is 0, 1, 2, 3 or 4, m6 is 0, 1, 2 or 3, m7 is 0 or 1, m8 is 1, 2, 3 or 4, m9 is 0, 1, 2 or 3, m10 is 0, 1, 2, 3 or 4, m11 is 0 or 1, m12 is 0 or 1, provided that 0≤m2+m3+m12≤4 when m1=0 and 0≤m2+m3+m12≤6 when m1=1; 0≤m5+m6≤4 when m4=0 and 0≤m5+m6≤6 when m4=1, 0≤m8+m9≤5 when m7=0 and 0≤m8+m9≤7 when m7=1, and 1≤m2+m5+m8≤4,
R^{A} is hydrogen, fluorine, methyl or trifluoromethyl,
R¹¹, R¹² and R¹³ are each independently halogen exclusive of iodine, nitro, cyano, hydroxy, carboxy, a C₁-C₂₀ hydrocarbyl group which may contain a heteroatom, C₁-C₂₀ hydrocarbyloxy group which may contain a heteroatom, C₁-C₂₀ hydrocarbylthio group which may contain a heteroatom or C₂-C₂₀ hydrocarbyloxycarbonyl group which may contain a heteroatom; when m3 is 2 or 3, a plurality of R¹¹ may be identical or different and two R¹¹ may bond together to form a ring with the carbon atoms to which they are attached; when m6 is 2 or 3, a plurality of R¹² may be identical or different and two R¹² may bond together to form a ring with the carbon atoms to which they are attached; when m9 is 2 or 3, a plurality of R¹³ may be identical or different and two R¹³ may bond together to form a ring with the carbon atoms to which they are attached;
L^{A}, L^{B}, L^{C}, L^{D} and L^{E} are each independently a single bond, ether bond, ester bond, sulfonate ester bond, amide bond, sulfonamide bond, carbonate bond or carbamate bond,
X^{L1} and X^{L2} are each independently a single bond or a C₁-C₄₀ hydrocarbylene group which may contain a heteroatom,
Rf¹ and Rf² are each independently a fluorine atom or a C₁-C₆ fluorinated saturated hydrocarbyl group,
Rf³ and Rf⁴ are each independently hydrogen, fluorine or a C₁-C₆ fluorinated saturated hydrocarbyl group,
provided that m11 and m12 are not simultaneously 0, and L^{A}, L^{B}, L^{C}, L^{D}, X^{L1} and X^{L2} are not simultaneously single bonds.

In another aspect, the invention provides a monomeric photoacid generator in the form of the sulfonium salt monomer defined herein.

In a further aspect, the invention provides a polymer comprising repeat units derived from the monomeric photoacid generator defined above.

The polymer may further comprise repeat units of at least one type selected from repeat units having the formula (a1), repeat units having the formula (a2), and repeat units having the formula (a3):

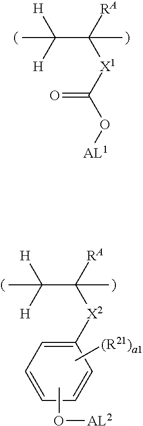

wherein R^{A} is each independently hydrogen, fluorine, methyl or trifluoromethyl,
X¹ is a single bond, phenylene group, naphthylene group, *—C(═O)—O—X¹¹— or *—C(═O)—N(H)—X¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O— or *—C(═O)—N(H)—,
* designates a point of attachment to the carbon atom in the backbone,
R²¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom; when a1 is 2, 3 or 4, a plurality of R²¹ may be identical or different,
AL¹ and AL² are each independently an acid labile group, and
a1 is 0, 1, 2, 3 or 4,

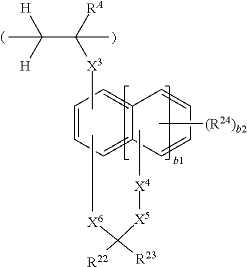

wherein b1 is 0 or 1, b2 is 0, 1, 2 or 3 when b1=0 and 0, 1, 2, 3, 4 or 5 when b1=1,
R^{A} is hydrogen, fluorine, methyl or trifluoromethyl,
X³ is a single bond, *—C(═O)—O— or *—C(═O)—N(H)—, * designates a point of attachment to the carbon atom in the backbone,
X⁴ is a single bond, a C₁-C₄ aliphatic hydrocarbylene group, carbonyl group, sulfonyl group or a group obtained by combining the foregoing,
X⁵ and X⁶ are each independently oxygen or sulfur, provided that X⁴ and X⁶ are attached to adjacent carbon atoms on the aromatic ring,
R²² and R²³ are each independently hydrogen or a C₁-C₂₀ hydrocarbyl group which may contain a heteroatom, R²² and R²³ may bond together to form a ring with the carbon atom to which they are attached,
R²⁴ is halogen, hydroxy, cyano, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{24A})(R^{24B}), R^{24A} and R^{24B} are each independently hydrogen or a C₁-C₆ hydrocarbyl group; when b2 is 2 or more, a plurality of R²⁴ may be identical or different and may bond together to form a ring with the carbon atom on the aromatic ring to which they are attached.

The polymer may further comprise repeat units of at least one type selected from repeat units having the formula (b1) and repeat units having the formula (b2):

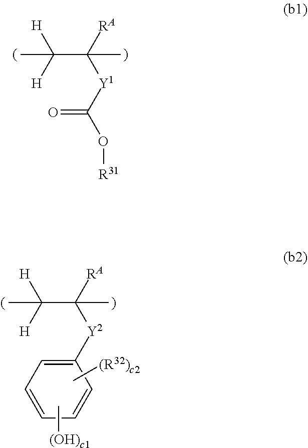

wherein R^{A} is each independently hydrogen, fluorine, methyl or trifluoromethyl,
Y¹ is a single bond, phenylene group, naphthylene group, *—C(═O)—O—Y¹¹— or *—C(═O)—N(H)—Y¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, Y¹¹ is a C₁-C₁₀ saturated hydrocarbylene group, phenylene group or naphthylene group, the saturated hydrocarbylene group may contain hydroxy, ether bond, ester bond or lactone ring,
Y² is a single bond or *—C(═O)—O—, * designates a point of attachment to the carbon atom in the backbone,
R³¹ is hydrogen or a C₁-C₂₀ group which contains at least one structure selected from hydroxy exclusive of phenolic hydroxy, cyano, nitro, carbonyl, carboxy, ether bond, ester bond, sulfonate ester bond, carbonate bond, lactone ring, sultone ring, and carboxylic anhydride (—C(═O)—O—C(═O)—),
R³² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom; when c2 is 2, 3 or 4, a plurality of R³² may be identical or different,
c1 is 1, 2, 3 or 4, c2 is 0, 1, 2, 3 or 4, provided that 1≤c1+c2≤5.

In a yet further aspect, the invention provides a chemically amplified resist composition comprising (A) a base polymer containing the polymer defined herein.

The chemically amplified resist composition may further comprise (B) an organic solvent.

The chemically amplified resist composition may further comprise (C) a quencher.

The chemically amplified resist composition may further comprise (D) a photoacid generator.

The chemically amplified resist composition may further comprise (E) a surfactant.

In a still further aspect, the invention provides a pattern forming process comprising the steps of applying the chemically amplified resist composition defined herein onto a substrate to form a resist film thereon, exposing the resist film to high-energy radiation, and developing the exposed resist film in a developer.

In a preferred embodiment, the high-energy radiation is KrF excimer laser, ArF excimer laser, EB or EUV of wavelength 3 to 15 nm.

### Advantageous Effects of the Invention

When a chemically amplified resist composition comprising a polymer comprising repeat units derived from a sulfonium salt monomer and functioning as a photoacid generator is processed by lithography, a resist pattern having a high sensitivity, high contrast and improved lithography properties including LWR, CDU, EL and DOF is formed.

### DESCRIPTION OF THE PREFERRED EMBODIMENT

Hereinafter, the invention is described in detail. It is understood that for some structures represented by chemical formulae, there can exist enantiomers and diastereomers because of the presence of asymmetric carbon atoms. In such a case, a single formula collectively represents all such isomers. The isomers may be used alone or in admixture.

### [Sulfonium Salt Monomer]

One embodiment of the invention is a sulfonium salt monomer having the formula (A).

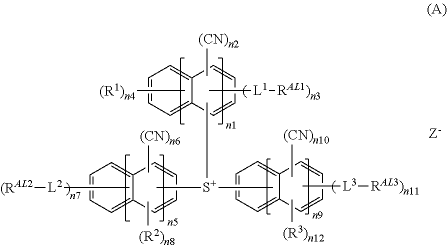

In formula (A), n1 is 0 or 1. The relevant structure is a benzene ring in case of n1=0 and a naphthalene ring in case of n1=1. From the aspect of solvent solubility, the benzene ring corresponding to n1=0 is preferred. n2 is 0, 1 or 2. n3 is 0, 1 or 2. n4 is 0, 1 or 2. It is preferred from the aspect of reactant availability that n4 be 0 or 1. Provided that 0≤n2+n3+n4≤5 when n1=0, and 0≤n2+n3+n4≤7 when n1=1.

In formula (A), n5 is 0 or 1. The relevant structure is a benzene ring in case of n5=0 and a naphthalene ring in case of n5=1. From the aspect of solvent solubility, the benzene ring corresponding to n5=0 is preferred. n6 is 0, 1 or 2. n7 is 0, 1 or 2. n8 is 0, 1 or 2. It is preferred from the aspect of reactant availability that n8 be 0 or 1. Provided that 0≤n6+n7+n8≤5 when n5=0, and 0≤n6+n7+n8≤7 when n5=1.

In formula (A), n9 is 0 or 1. The relevant structure is a benzene ring in case of n9=0 and a naphthalene ring in case of n9=1. From the aspect of solvent solubility, the benzene ring corresponding to n9=0 is preferred. n10 is 0, 1 or 2. n11 is 0, 1 or 2. n12 is 0, 1 or 2. It is preferred from the aspect of reactant availability that n12 be 0 or 1. Provided that 0≤n10+n11+n12≤5 when n9=0, and 0≤n10+n11+n12≤7 when n9=1.

In formula (A), 1≤n2+n6+n10≤6, preferably 1≤n2+n6+n10≤3, more preferably 1≤n2+n6+n10≤2. 1≤n3+n7+n11≤6, preferably 1≤n3+n7+n11≤3, more preferably 1≤n3+n7+n11≤2.

In formula (A), R¹, R² and R³ are each independently halogen, nitro, hydroxy, carboxy, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Suitable halogen atoms include fluorine, chlorine, bromine and iodine. The hydrocarbyl group and hydrocarbyl moiety in the hydrocarbyloxy and hydrocarbylthio groups may be saturated or unsaturated, and may be straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. Inter alia, aryl groups are preferred. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety.

Two R¹ may be identical or different when n4=2. When n4=2, two R¹ may bond together to form a ring with the carbon atom to which they are attached. The ring is preferably 5 to 8-membered. Two R² may be identical or different when n8=2. When n8=2, two R² may bond together to form a ring with the carbon atom to which they are attached. The ring is preferably 5 to 8-membered. Two R³ may be identical or different when n12=2. When n12=2, two R³ may bond together to form a ring with the carbon atom to which they are attached. The ring is preferably 5 to 8-membered.

In formula (A), L¹, L² and L³ are each independently an ether bond, ester bond or carbonate bond. Inter alia, an ether bond or ester bond is preferred. Two L¹ may be identical or different when n3=2. Two L² may be identical or different when n7=2. Two L³ may be identical or different when n11=2.

In formula (A), R^{AL1}, R^{AL2} and R^{AL3} are each independently an acid labile group. Two R^{AL1} may be identical or different when n3=2. Two R^{AL2} may be identical or different when n7=2. Two R^{AL3} may be identical or different when n11=2.

The acid labile group is preferably a group having the formula (AL-1) or (AL-2).

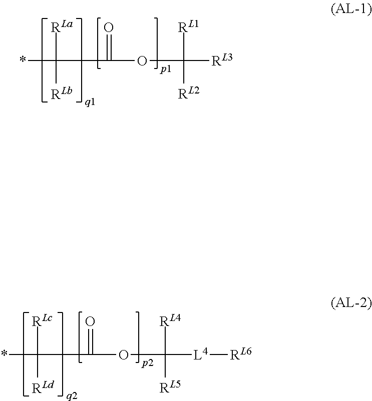

Herein, * designates a point of attachment to L¹, L² and L³.

In formulae (AL-1) and (AL-2), p1 and p2 are each independently 0 or 1. q1 and q2 are each independently 0, 1, 2, 3 or 4. When p1=1, q1 is preferably 1 or 2, and when p1=0, q1 is 0. When p2=1, q2 is preferably 1 or 2, and when p2=0, q2 is 0.

In formula (AL-1), R^{L1} and R^{L2} are each independently a C₁-C₁₂ hydrocarbyl group. R^{L3} is hydrogen or a C₁-C₁₂ hydrocarbyl group. Some constituent —CH₂— in the hydrocarbyl groups R^{L1}, R^{L2} and R^{L3} may be replaced by —O— or —S—, and when hydrocarbyl group contains an aromatic ring, some or all of the hydrogen atoms on the aromatic ring may be substituted by halogen, cyano, nitro, a C₁-C₄ alkyl group which may contain a halogen atom or C₁-C₄ alkoxy group which may contain a halogen atom.

The C₁-C₁₂ hydrocarbyl groups R^{L1}, R^{L2} and R^{L3} may be saturated or unsaturated, and may be straight, branched or cyclic. Examples thereof include C₁-C₁₂ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, n-decyl, n-undecyl, and n-dodecyl; C₃-C₁₂ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₁₂ alkenyl groups such as vinyl, 1-propenyl, 2-propenyl, butenyl, pentenyl, and hexenyl; C₂-C₁₂ alkynyl groups such as ethynyl, propynyl, butynyl, pentynyl, and hexynyl; C₃-C₁₂ cyclic unsaturated aliphatic hydrocarbyl groups such as cyclopentenyl and cyclohexenyl; C₆-C₁₂ aryl groups such as phenyl, naphthyl, and indanyl; C₇-C₁₂ aralkyl groups such as benzyl, 1-phenylethyl, and 2-phenylethyl; and combinations thereof.

R^{L1} and R^{L2} may bond together to form a ring with the carbon atom to which they are attached, and some constituent —CH₂— in the ring may be replaced by —O— or —S—. Examples of the ring formed at this time include cyclopropane, cyclobutane, cyclopentane, cyclohexane, cycloheptane, cyclooctane, norbornane, adamantane, tricyclo[5.2.1.0^{2,6}]decane and tetracyclo[5.2.1.0^{2,6}]]dodecane rings. Some constituent —CH₂— in the ring may be replaced by —O— or —S—. Provided that when R^{L3} is hydrogen, R^{L1} and R^{L2} bond together to form an alicyclic ring containing a multiple bond together with the carbon atom to which they are bonded. Some of the hydrogen atoms on the alicyclic ring may be replaced by halogen.

In formula (AL-2), R^{L4} and R^{L5} are each independently hydrogen or a C₁-C₁₀ hydrocarbyl group. The C₁-C₁₀ hydrocarbyl groups R^{L4} and R^{L5} may be saturated or unsaturated, and may be straight, branched or cyclic. Examples thereof are as exemplified above for the C₁-C₁₀ hydrocarbyl groups R^{L1}, R^{L2} and R^{L3}.

In formula (AL-2), R^{L6} is a C₁-C₂₀ hydrocarbyl group in which some constituent —CH₂— may be replaced by —O— or —S—. The hydrocarbyl group may be saturated or unsaturated, and may be straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, norbornylmethyl, adamantyl, adamantylmethyl, tricyclo[5.2.1.0^{2,6}]decyl, and tetracyclo[6.2.1.1^{3,6}.0^{2,7}]dodecyl; C₂-C₂₀ alkenyl groups such as vinyl, propenyl, butenyl, pentenyl, and hexenyl; C₂-C₂₀ alkynyl groups such as ethynyl, propynyl, butynyl, pentynyl, and hexynyl; C₃-C₂₀ cyclic unsaturated aliphatic hydrocarbyl groups such as cyclopentenyl, cyclohexenyl, and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl; and combinations thereof. R^{L5} and R^{L6} may bond together to form a C₃-C₂₀ heterocyclic group with the carbon atom and L⁴ to which they are attached, and some constituent —CH₂— in the heterocyclic group may be replaced by —O— or —S—.

In formula (AL-2), L⁴ is —O— or —S—.

In formulae (AL-1) and (AL-2), R^{La} to R^{Ld} are each independently hydrogen or a C₁-C₂₀ hydrocarbyl group which may contain a heteroatom. The C₁-C₂₀ hydrocarbyl group may be saturated or unsaturated, and may be straight, branched or cyclic. Examples thereof are as exemplified above for the hydrocarbyl group R^{L6}, but are not limited thereto.

Examples of the acid labile group having formula (AL-1) are shown below, but are not limited thereto. Herein, * designates a point of attachment to L¹, L² and L³.

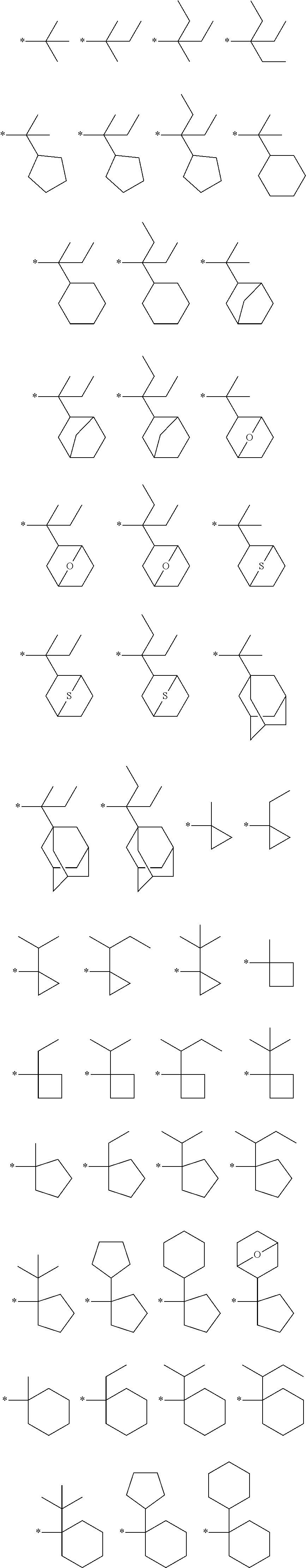

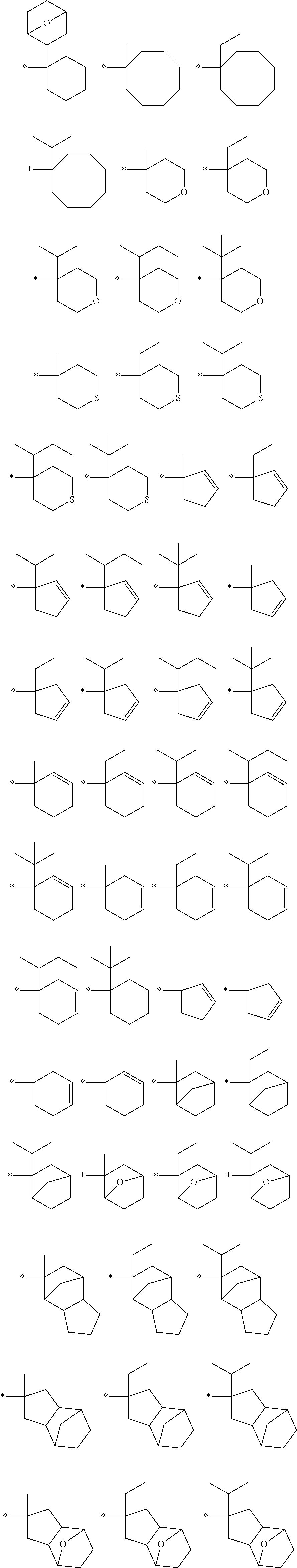

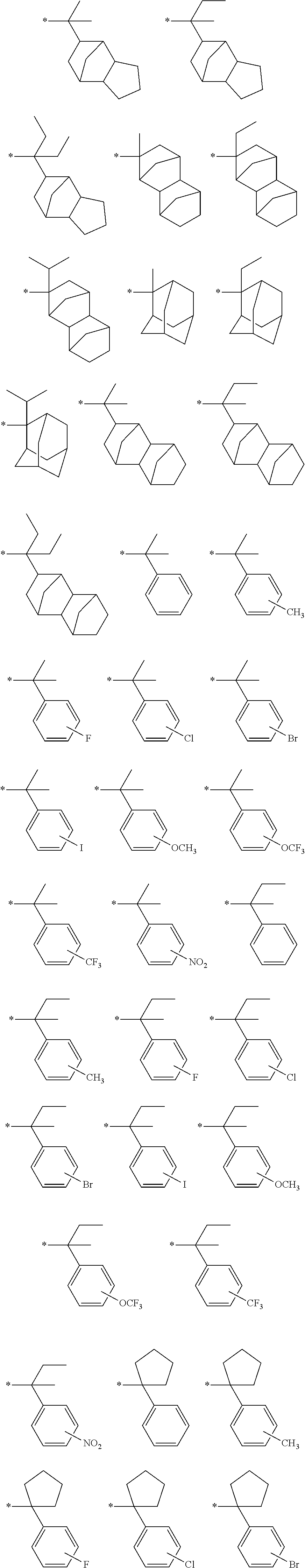

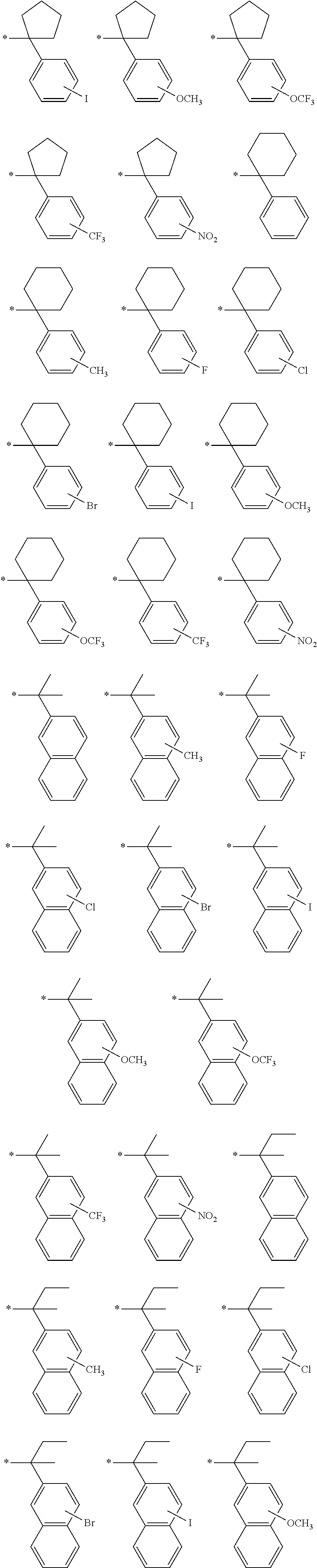

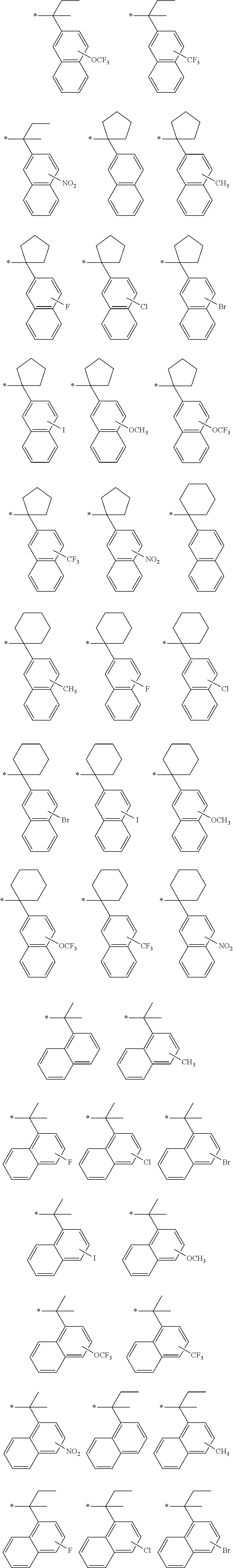

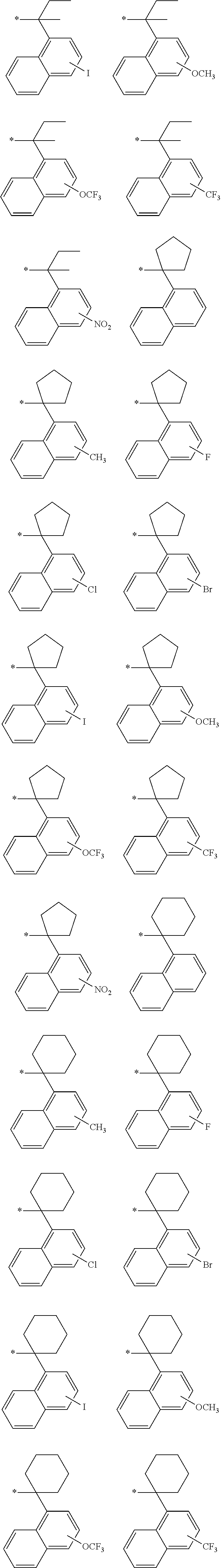

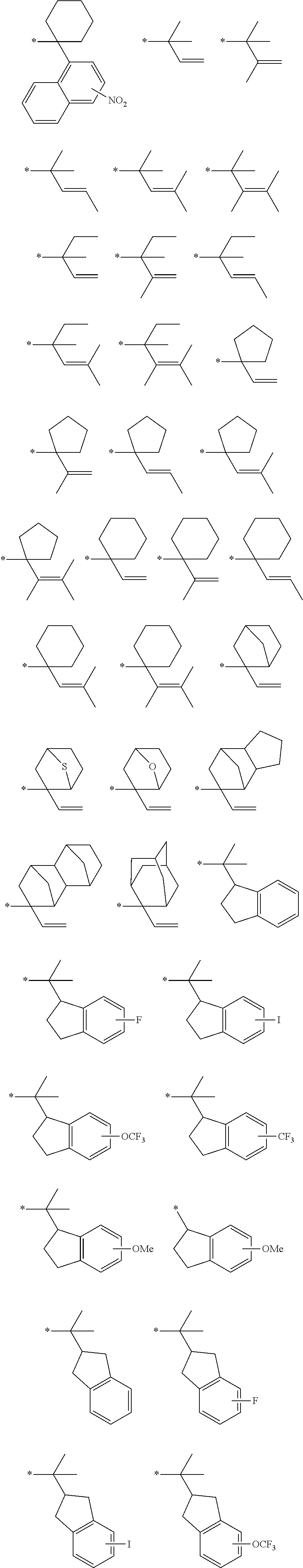

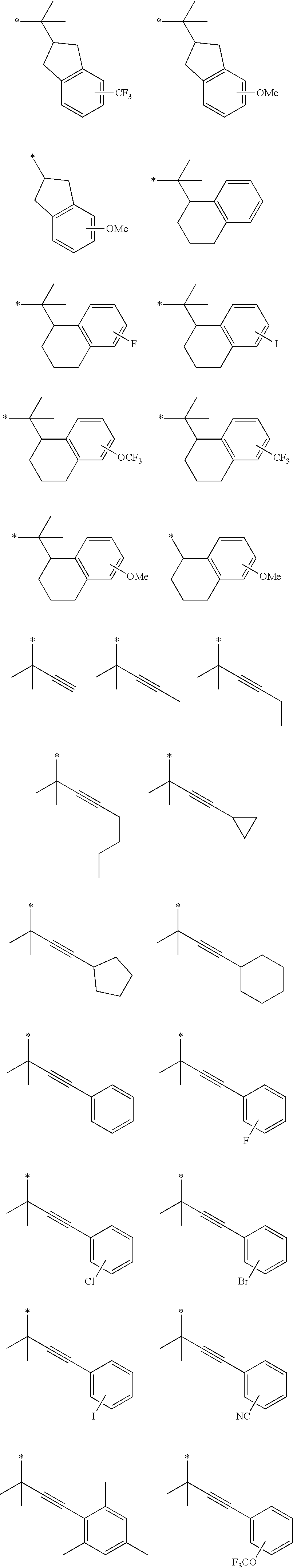

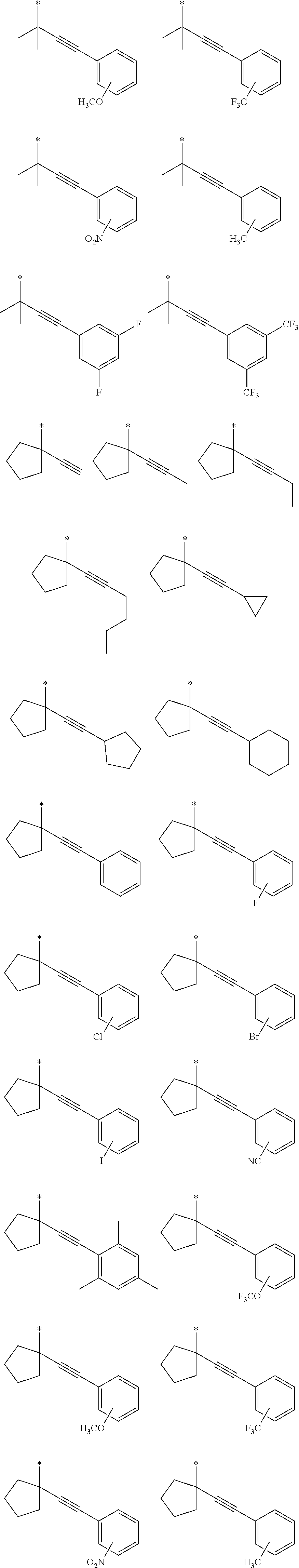

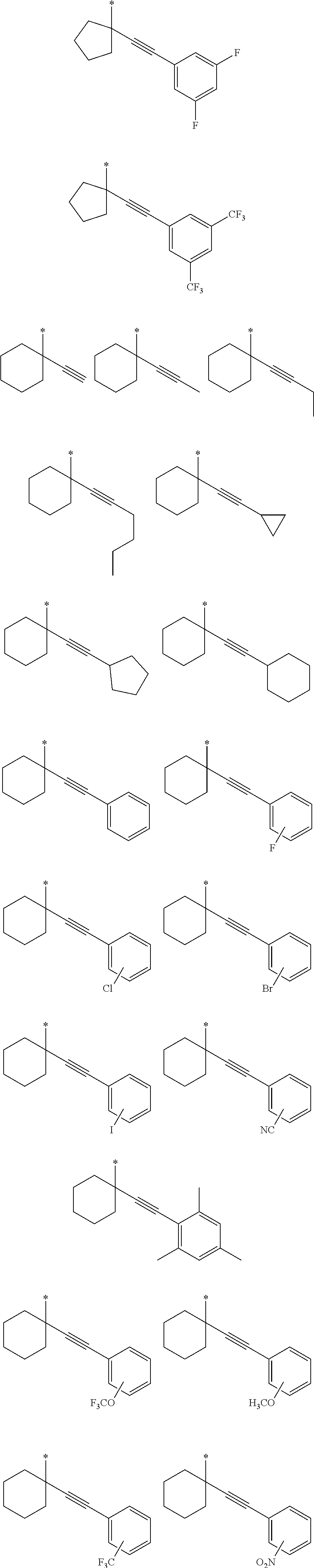

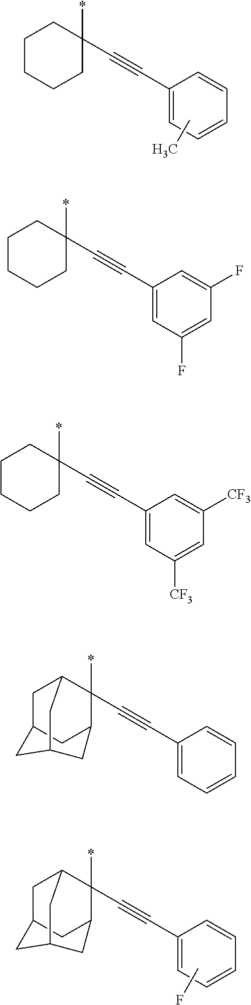

Examples of the acid labile group having formula (AL-2) are shown below, but are not limited thereto. Herein, * designates a point of attachment to L¹, L² and L³.

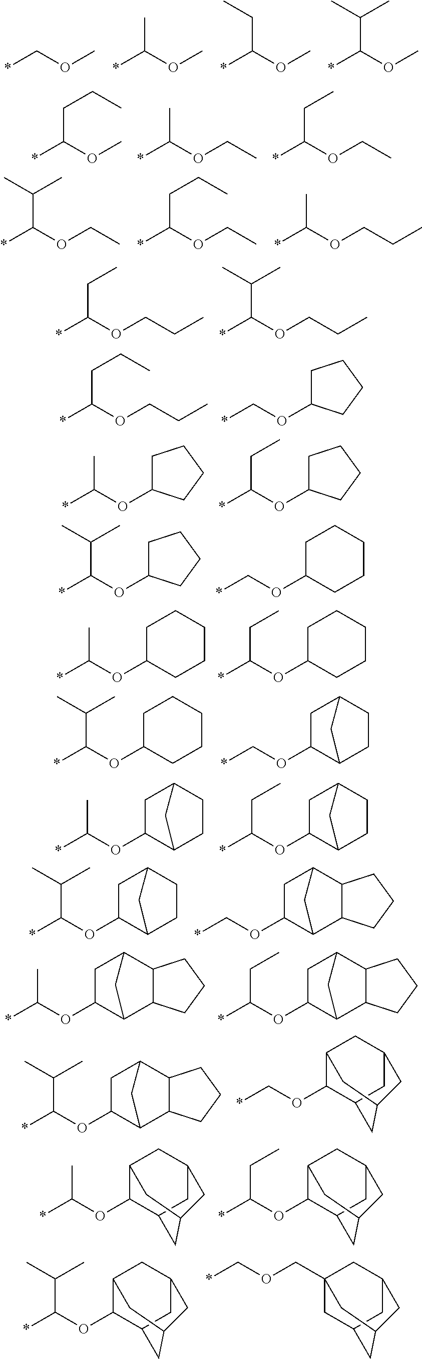

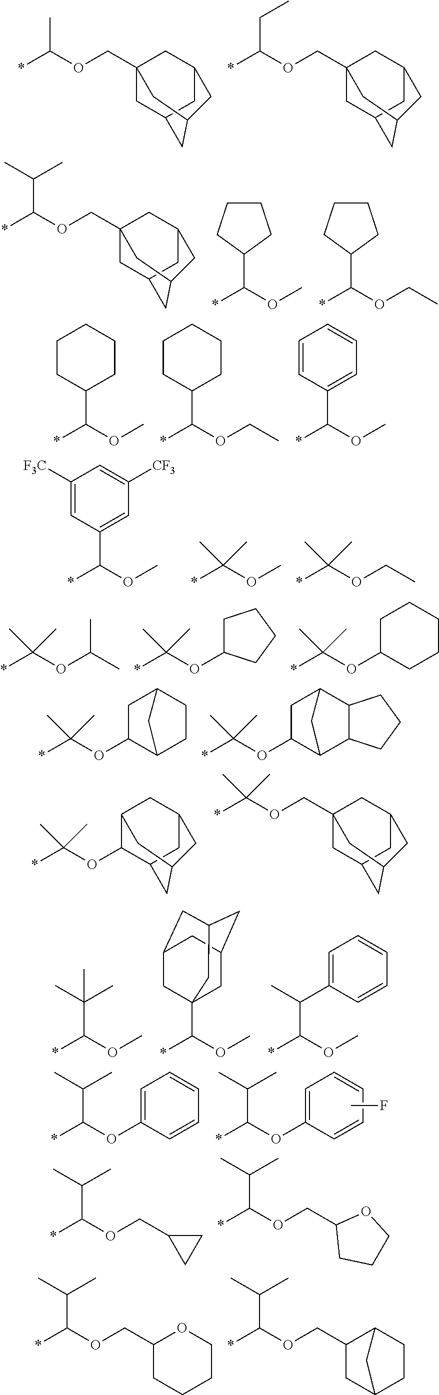

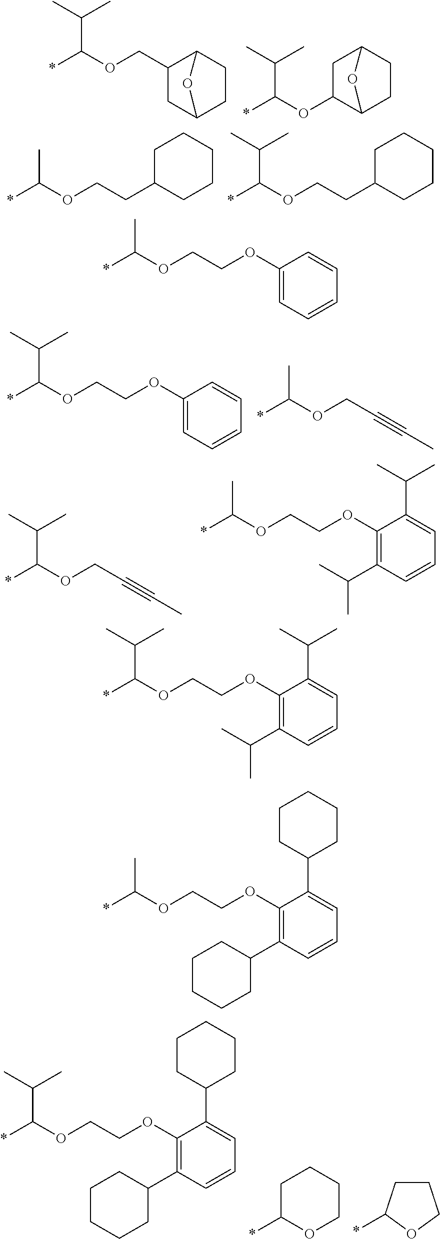

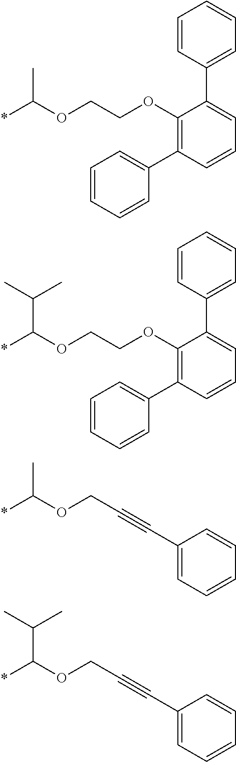 [SMILES: *C(C)OCCOc1c(-c2ccccc2)cccc1-c1ccccc1 ; *C(OCCOc1c(-c2ccccc2)cccc1-c1ccccc1)C(C)C ; *C(C)OCC#Cc1ccccc1 ; *C(OCC#Cc1ccccc1)C(C)C]

In formula (A), the cyano and -L¹-R^{AL1} are preferably bound to carbon atoms adjacent to each other. When the cyano and -L¹-R^{AL1} are adjacent to each other, the acidity of phenols and carboxylic acids formed by deprotection of —R^{AL1} is increased by the electron-withdrawing property of the cyano, so that the dissolution contrast is improved.

In formula (A), the cyano and -L²-R^{AL2} are preferably bound to carbon atoms adjacent to each other. When the cyano and -L²-R^{AL2} are adjacent to each other, the acidity of phenols and carboxylic acids formed by deprotection of —R^{AL2} is increased by the electron-withdrawing property of the cyano, so that the dissolution contrast is improved.

In formula (A), the cyano and -L³-R^{AL3} are preferably bound to carbon atoms adjacent to each other. When the cyano and -L³-R^{AL3} are adjacent to each other, the acidity of phenols and carboxylic acids formed by deprotection of —R^{AL3} is increased by the electron-withdrawing property of the cyano, so that the dissolution contrast is improved.

Two of three aromatic rings bonded to S⁺ may bond together to form a ring with the sulfur atom to which they are attached. Exemplary structures of the ring are shown below.

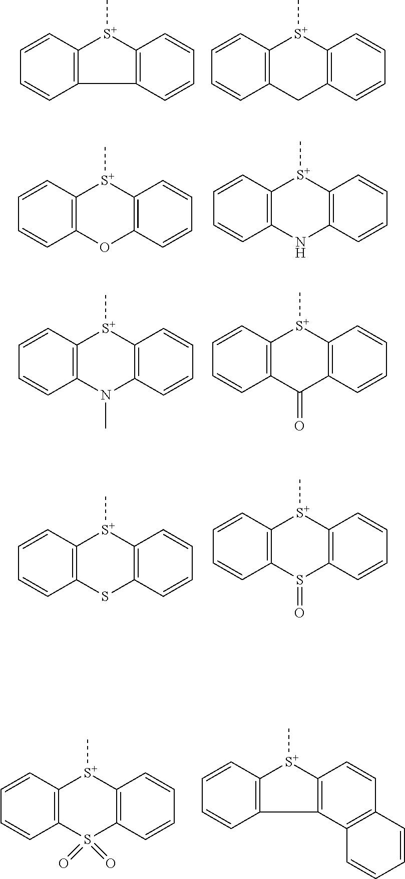

Herein, the broken line denotes a point of attachment.

Of the sulfonium salt monomers having formula (A), a monomer having the formula (A1) is preferred.

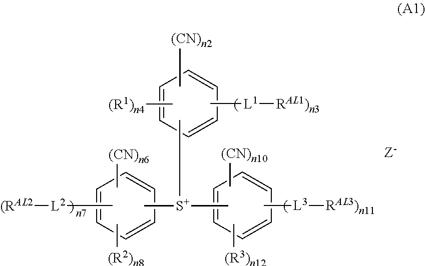

Herein, n2 to n4, n6 to n8, n10 to n12, R¹ to R³, L¹ to L³ and R^{AL1} to R^{AL3} are as defined above. Z⁻ is described below.

Examples of the cation in the sulfonium salt monomer having formula (A) are shown below, but are not limited thereto. Herein, Me is methyl.

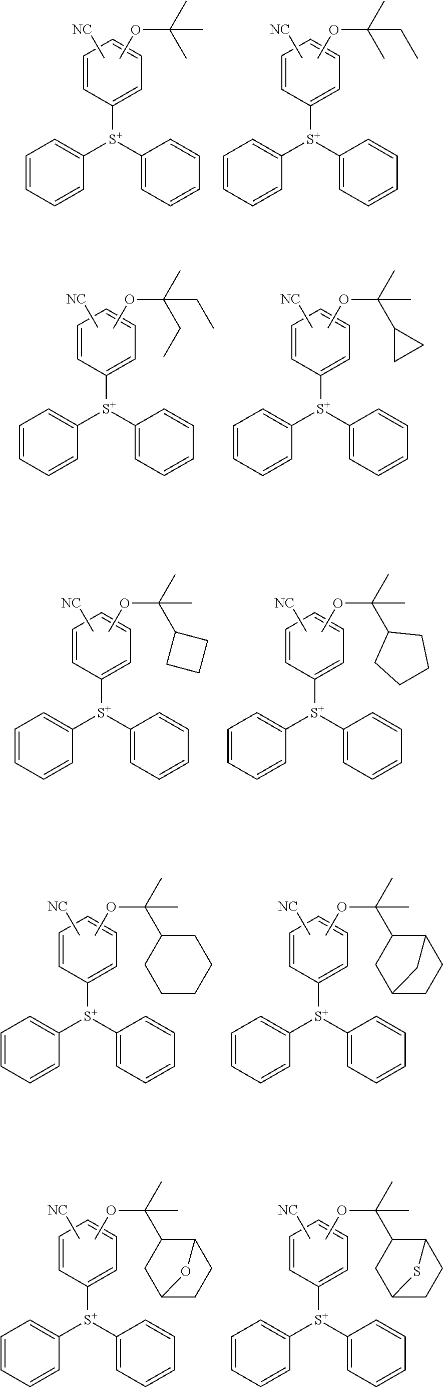

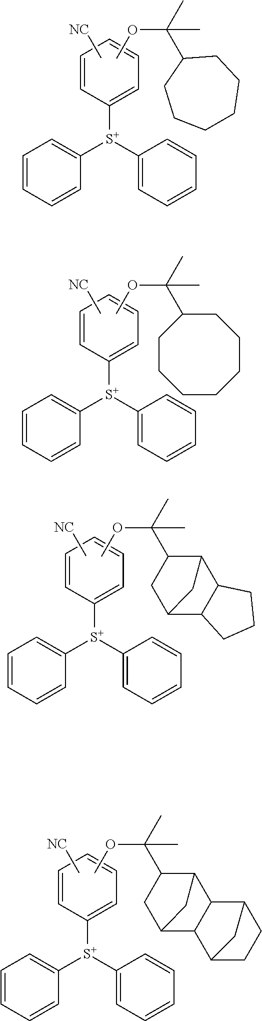

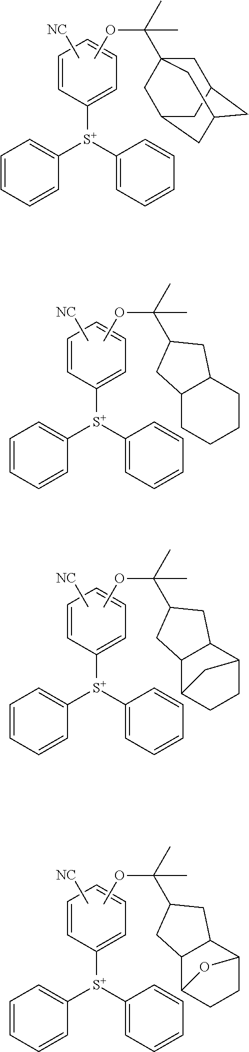

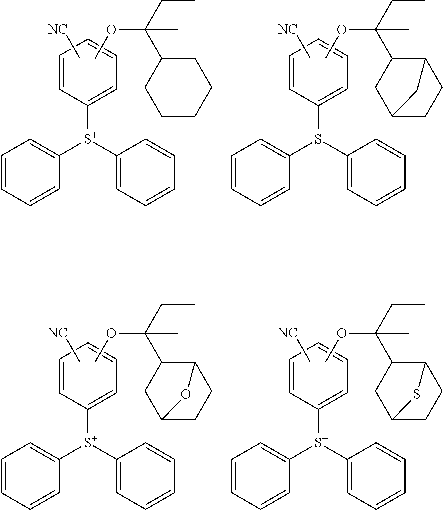

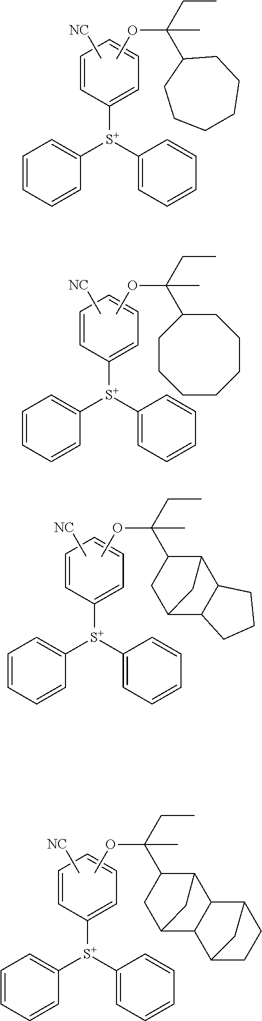

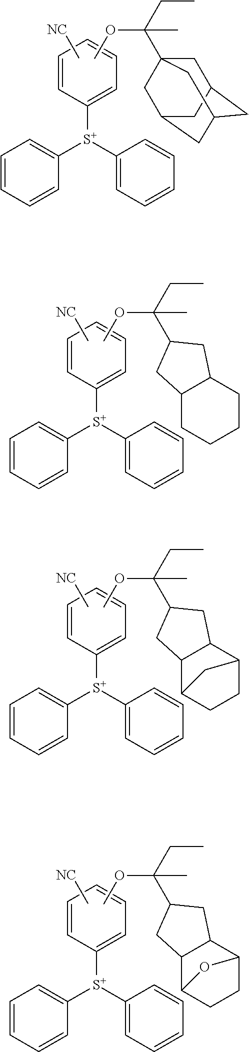

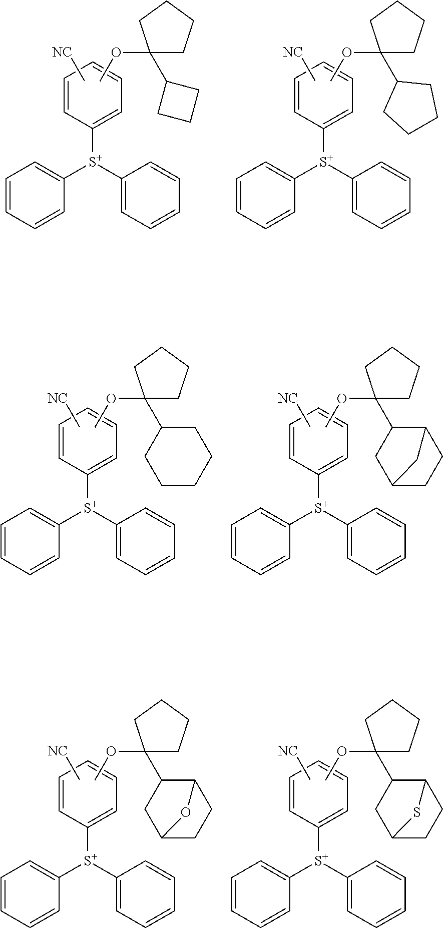

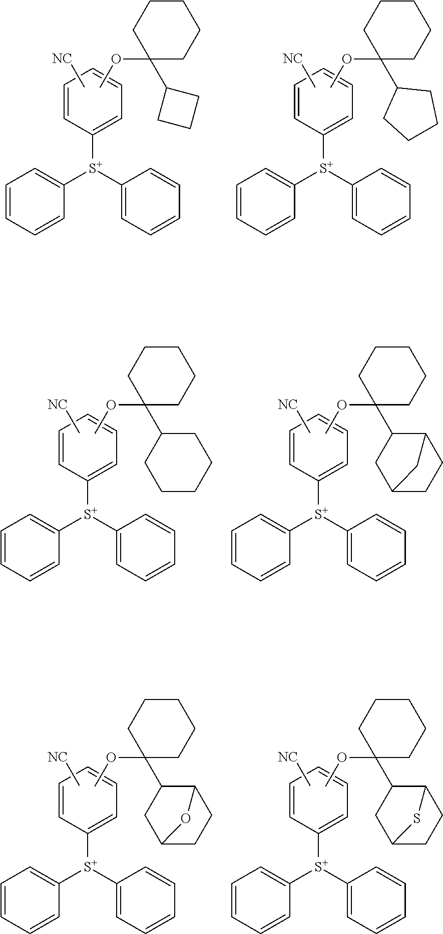

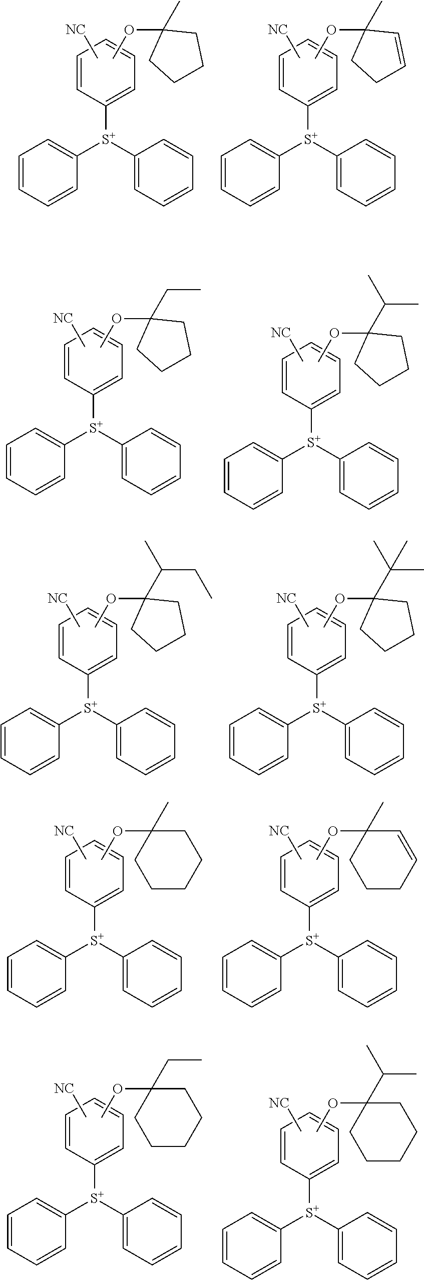

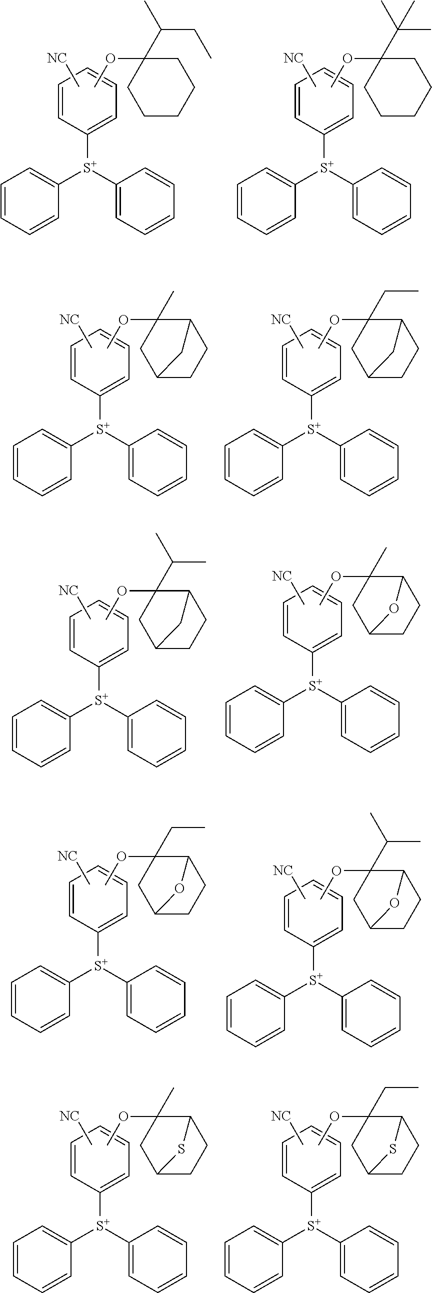

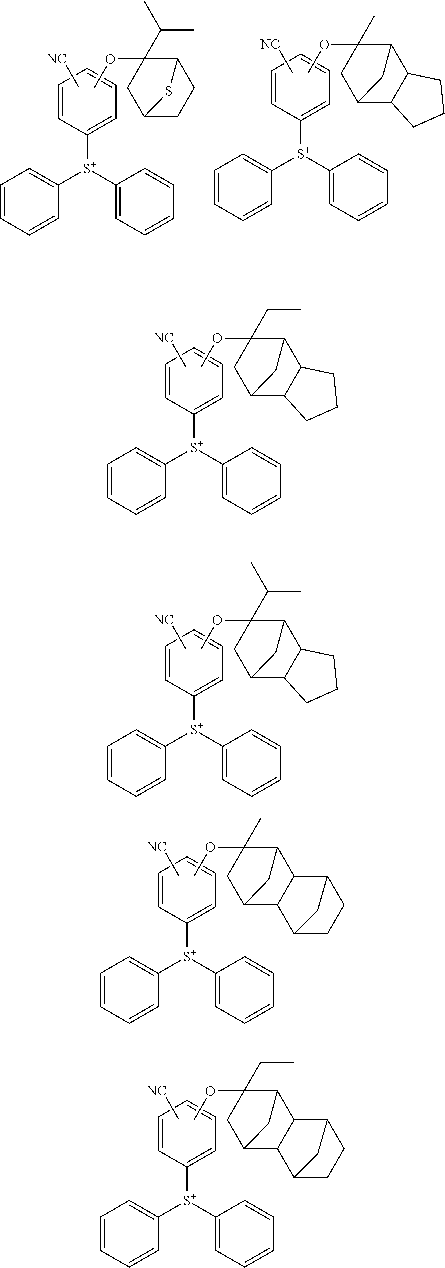

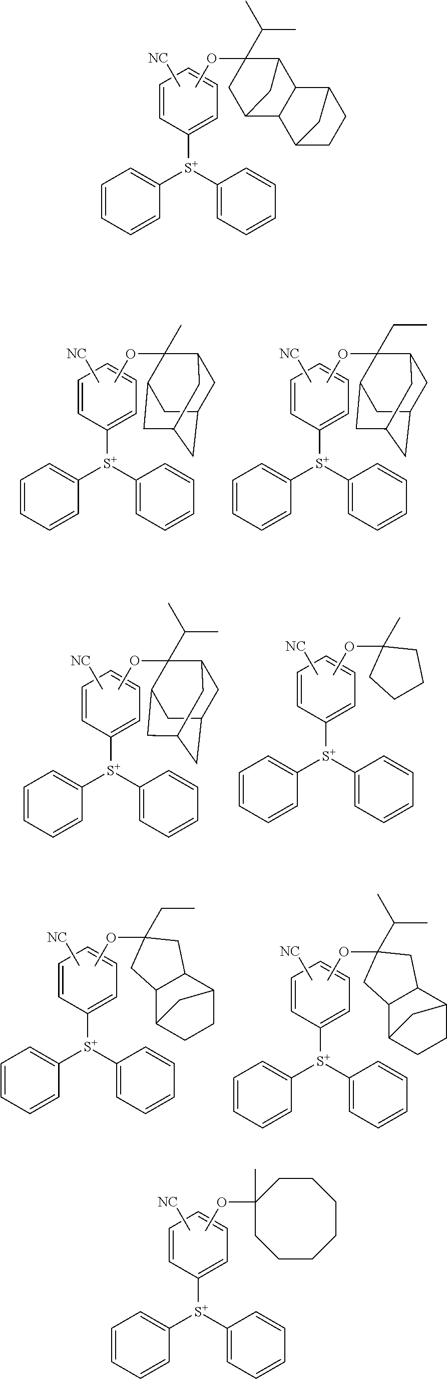

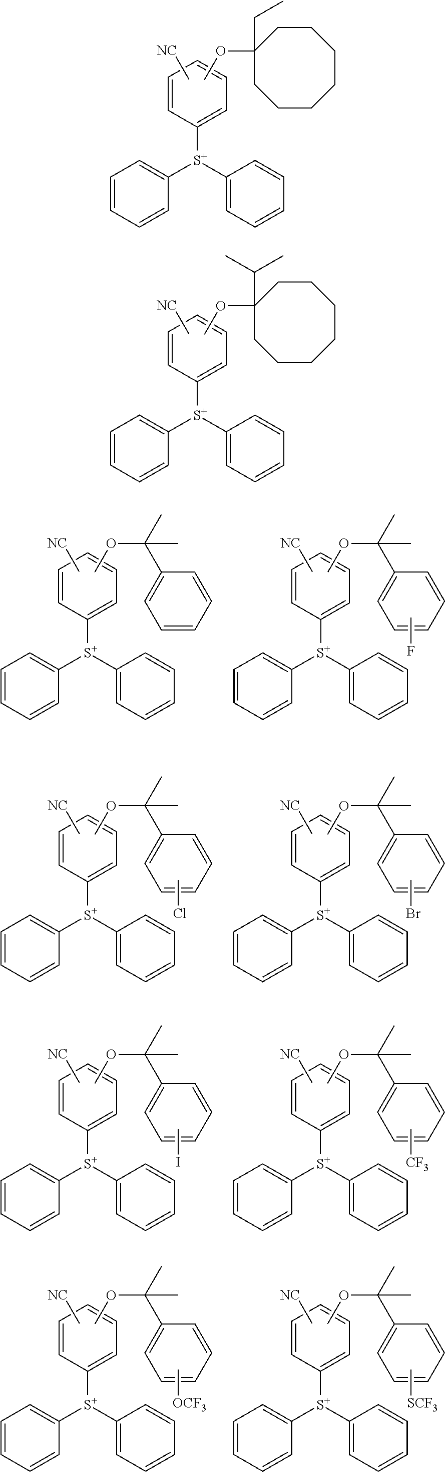

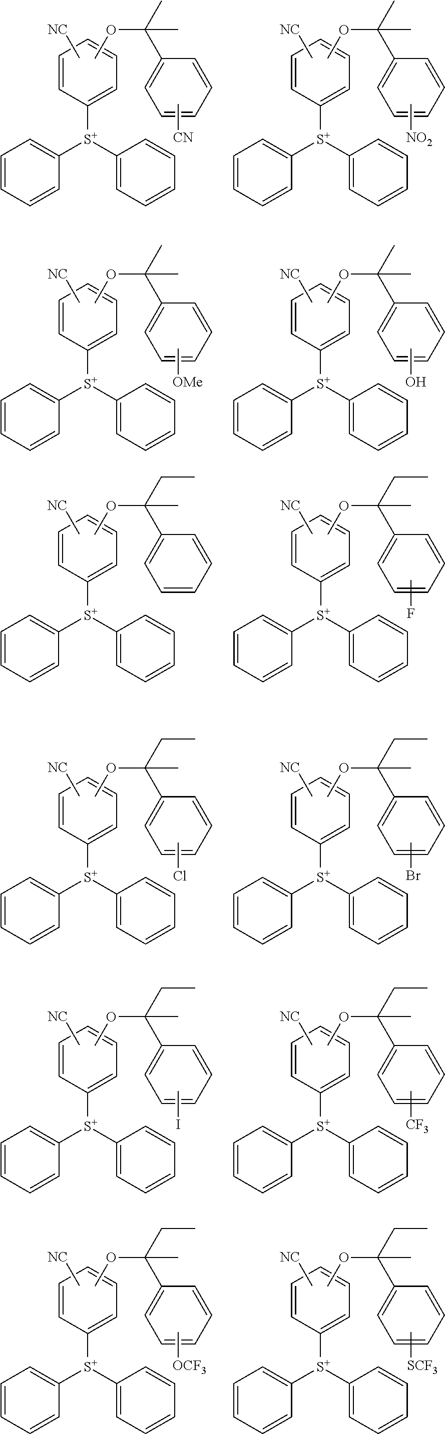

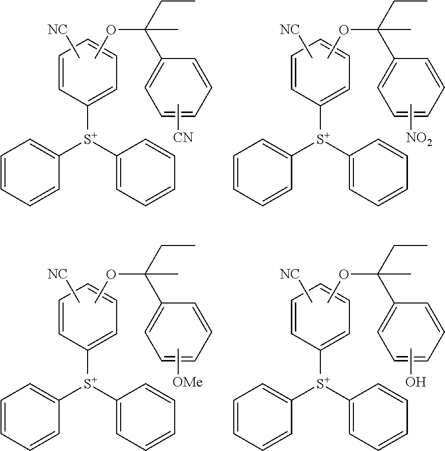

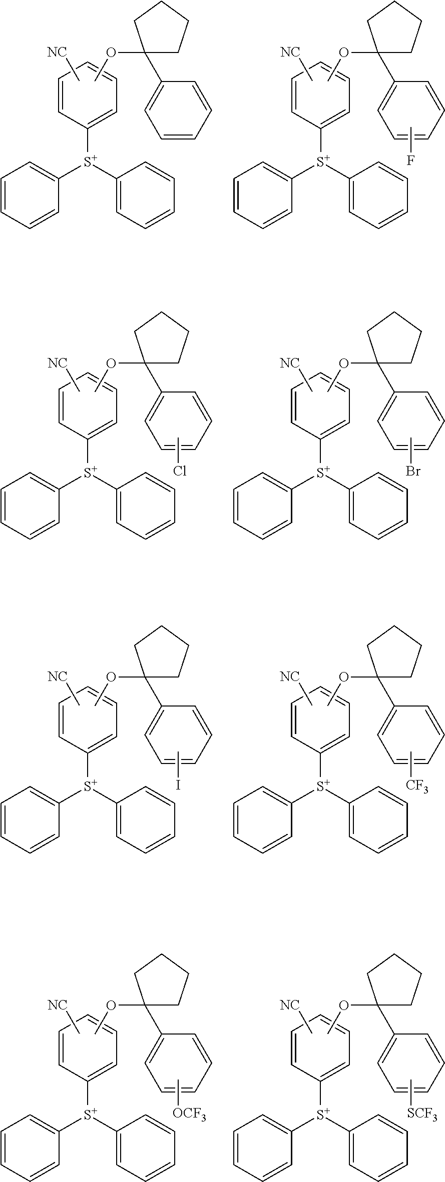

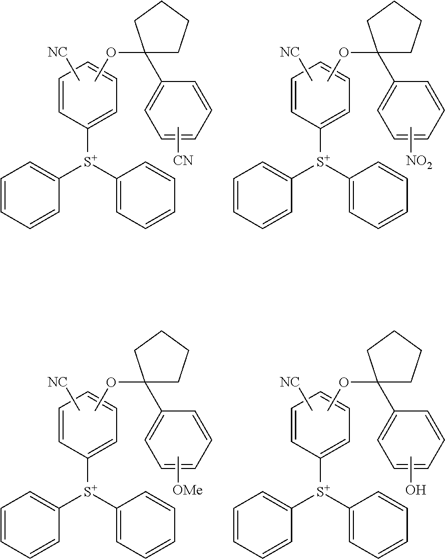

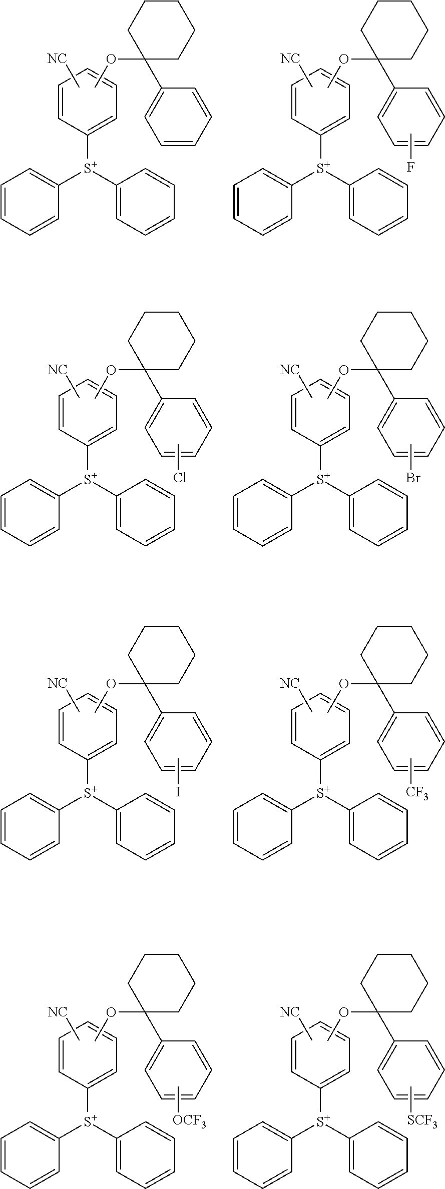

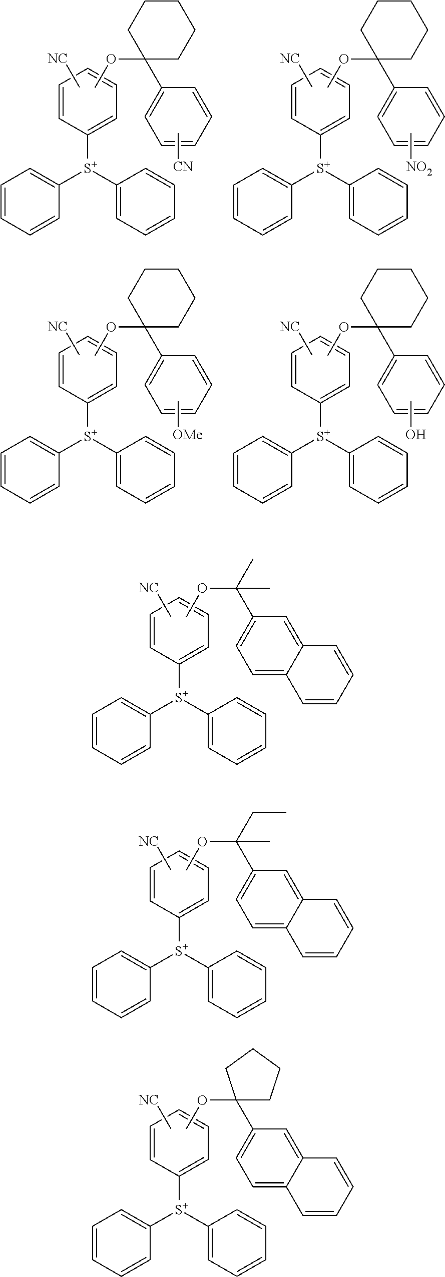

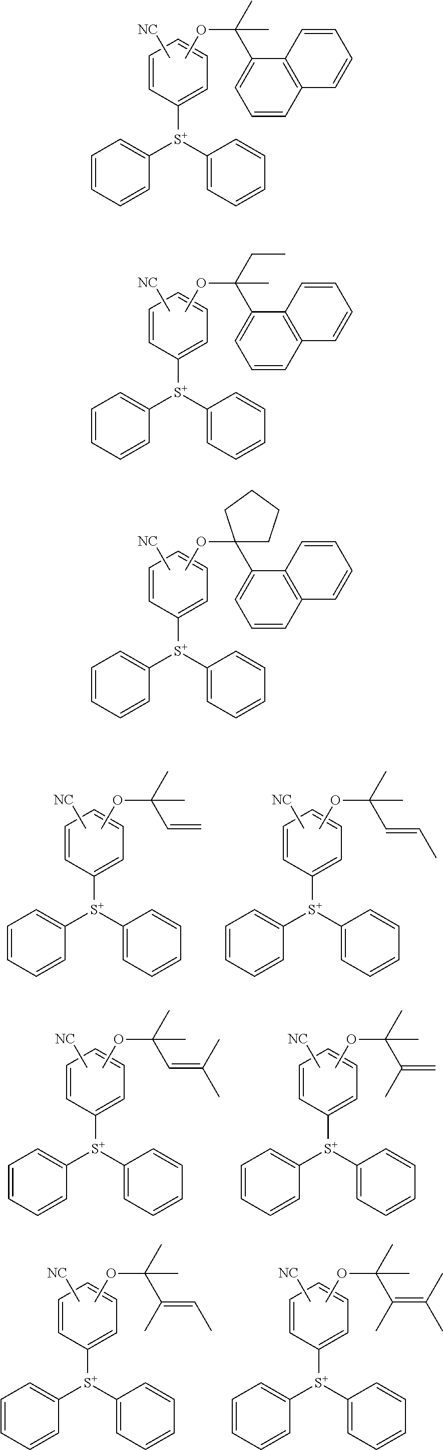

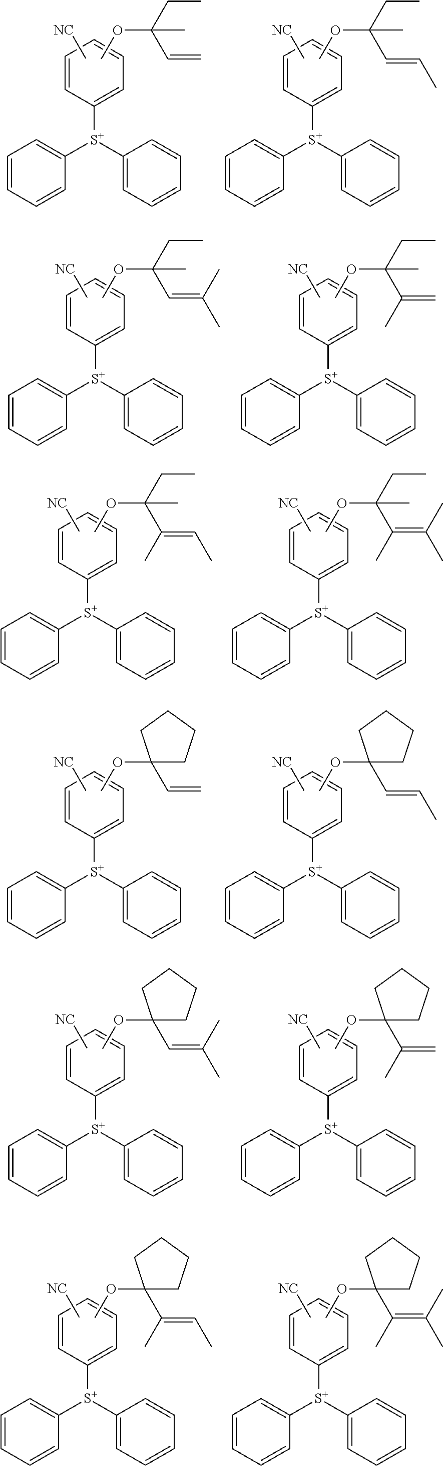

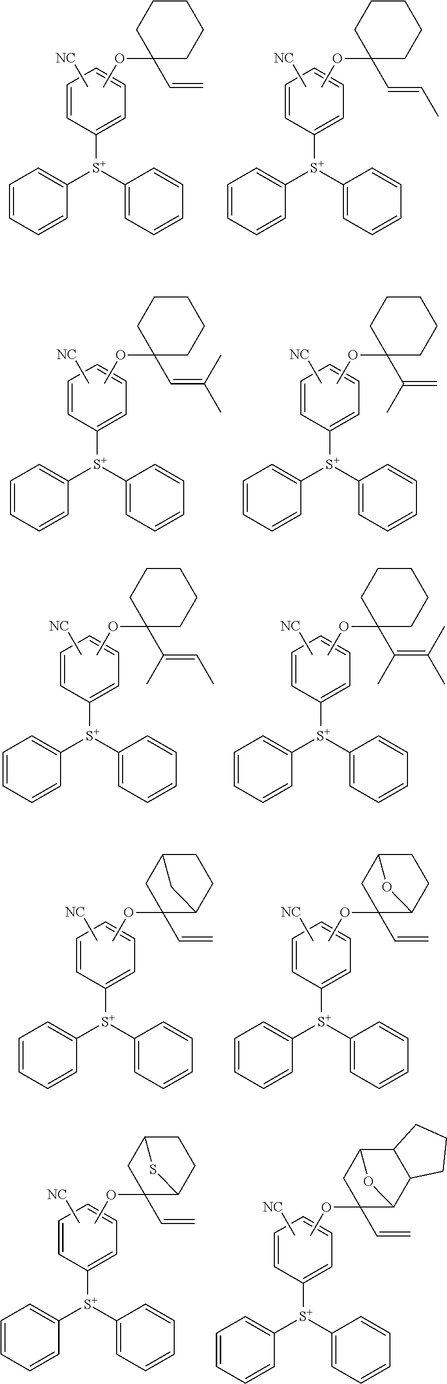

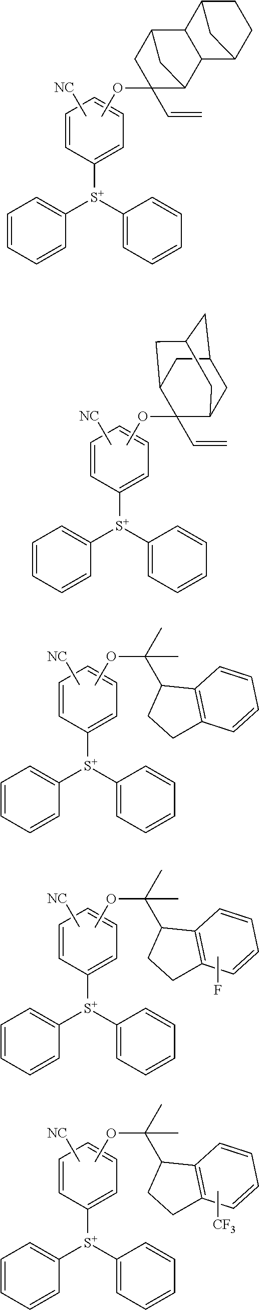

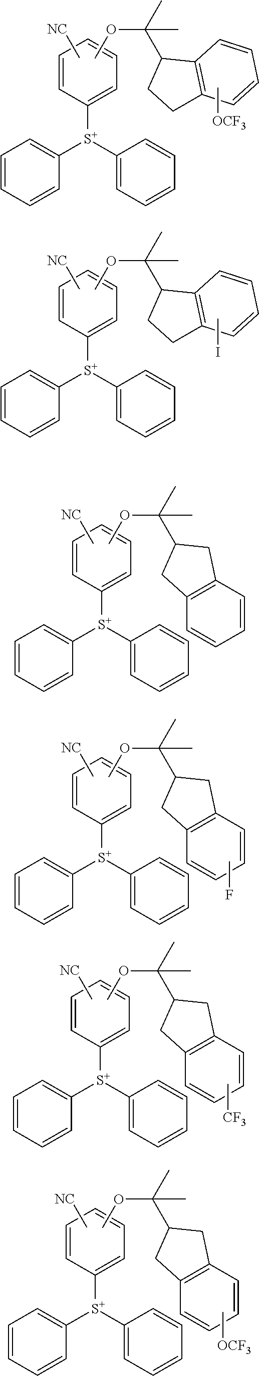

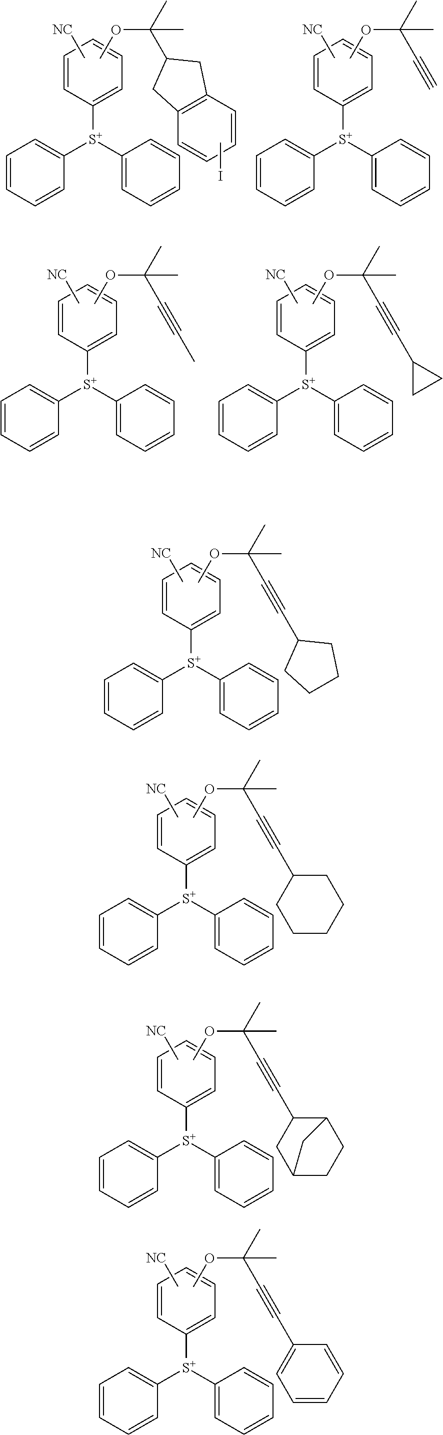

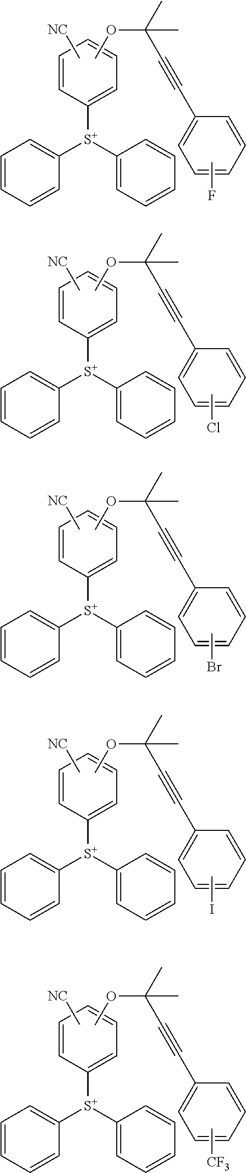

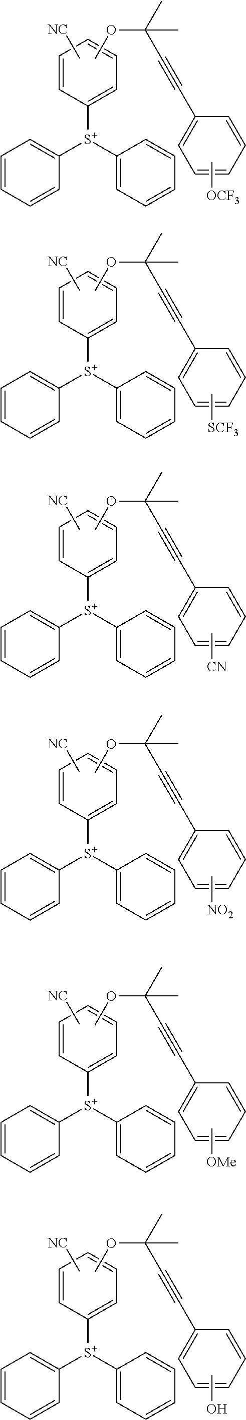

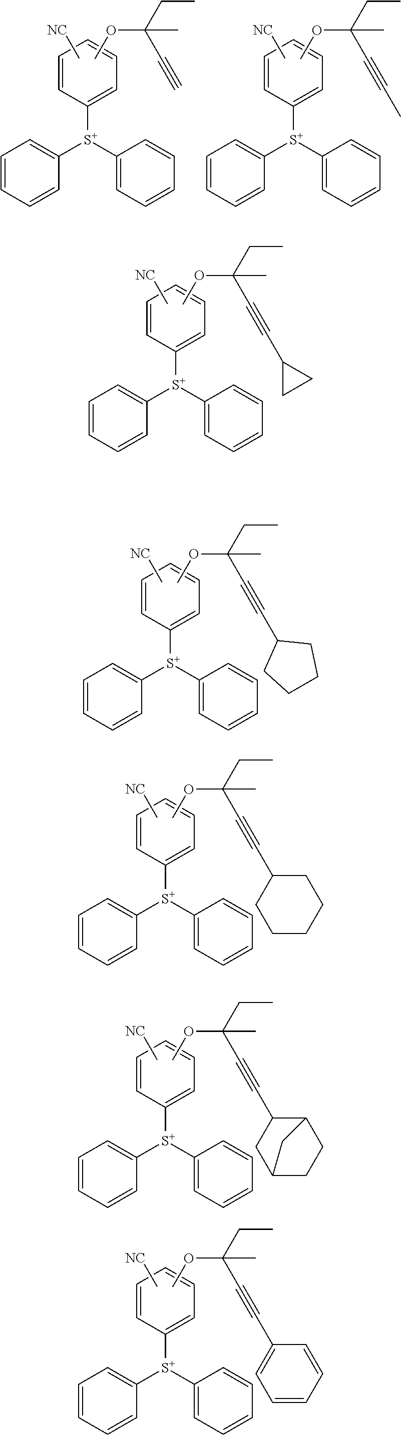

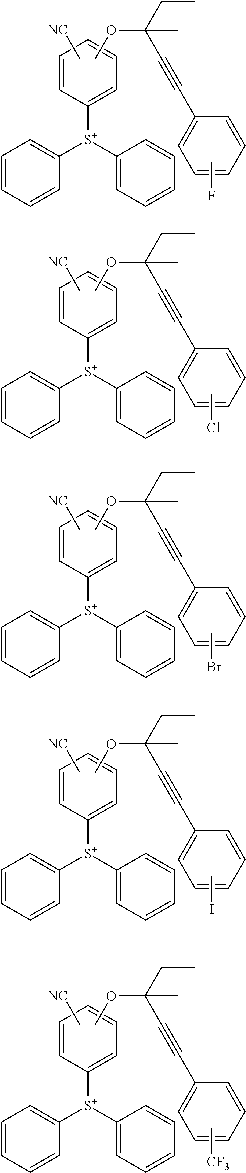

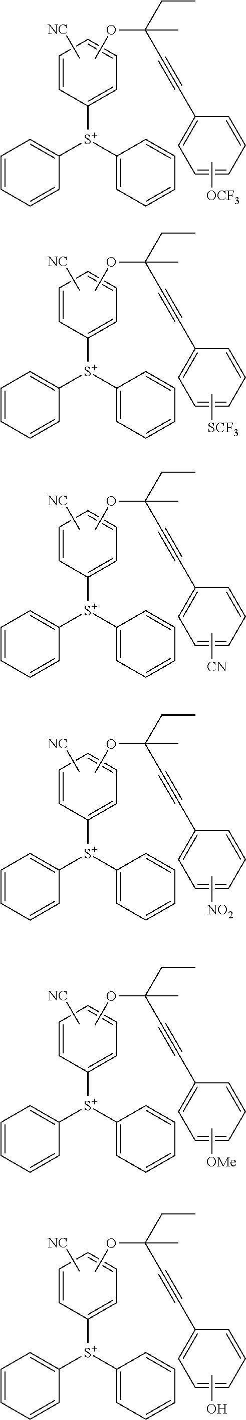

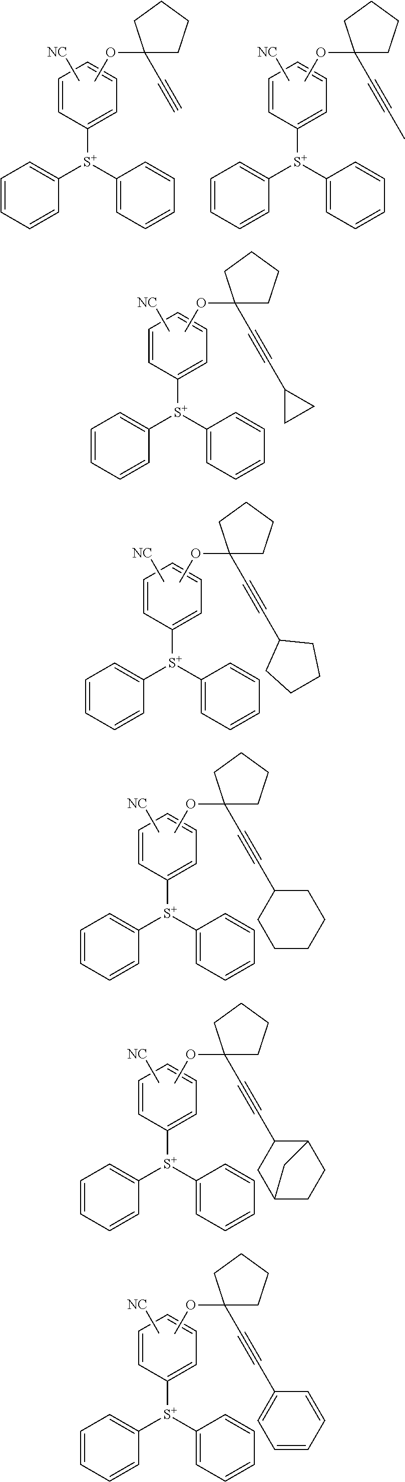

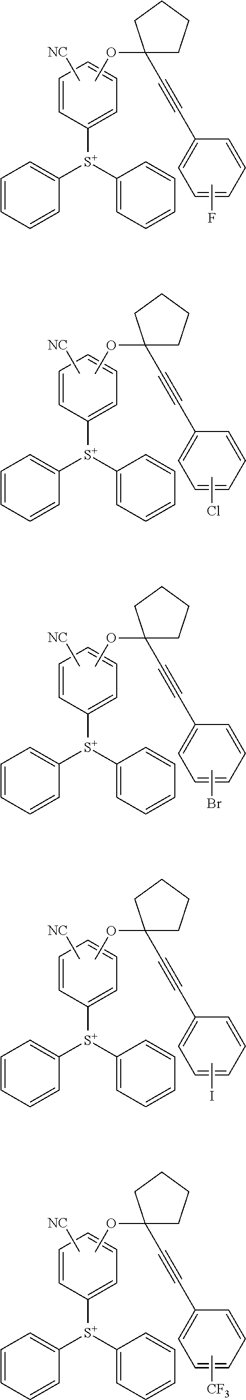

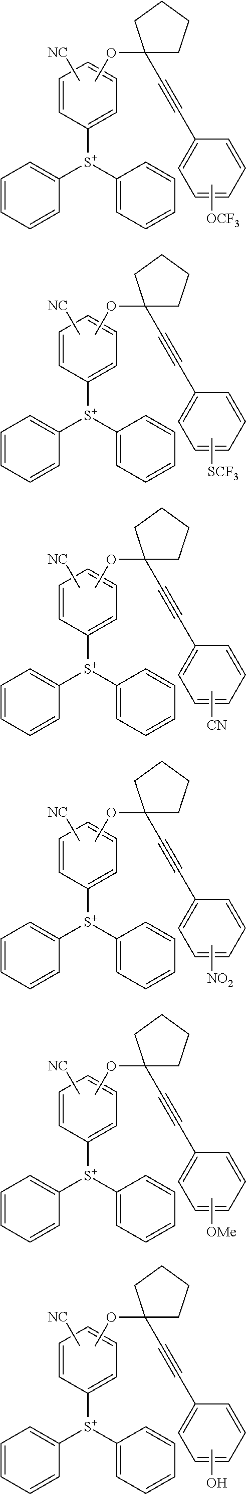

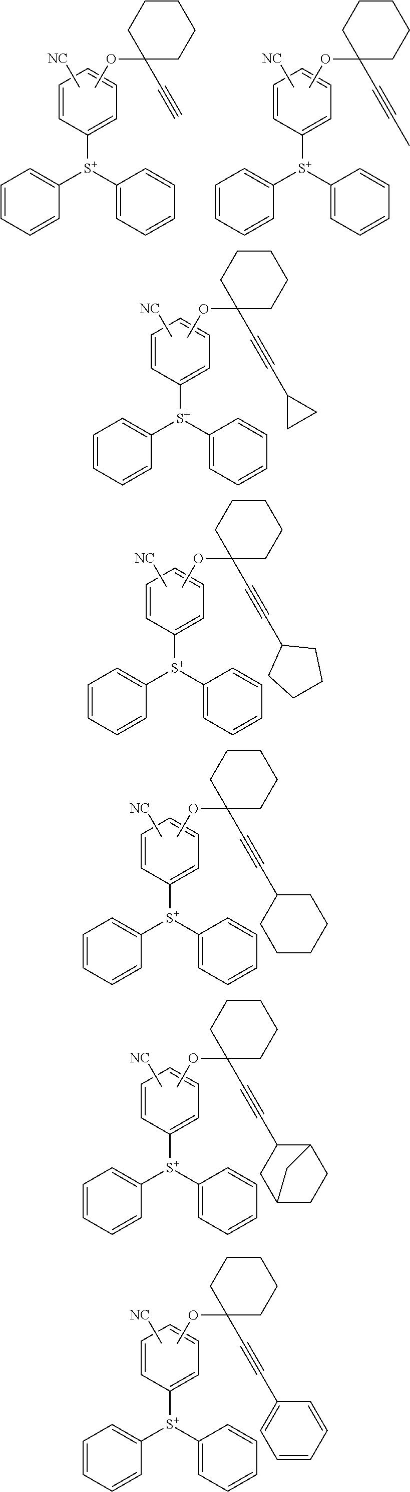

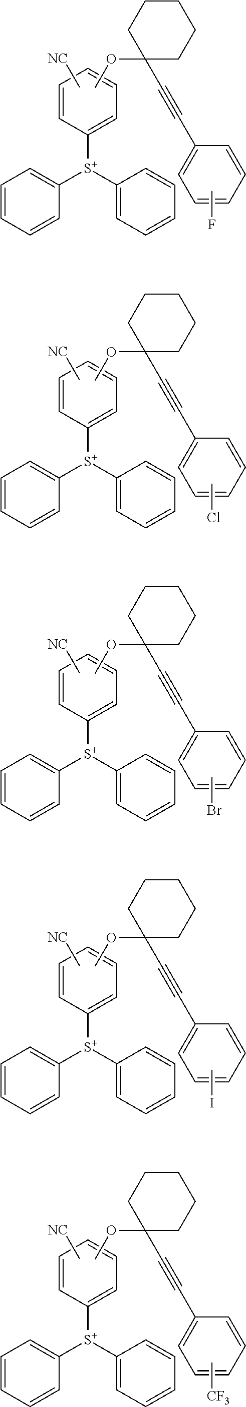

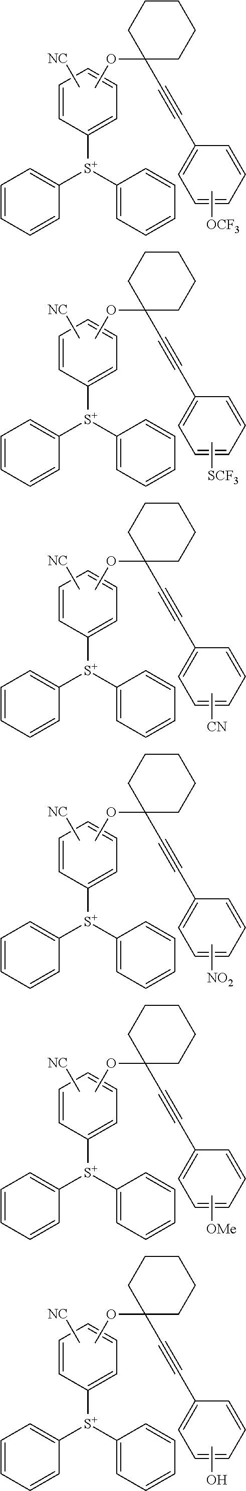

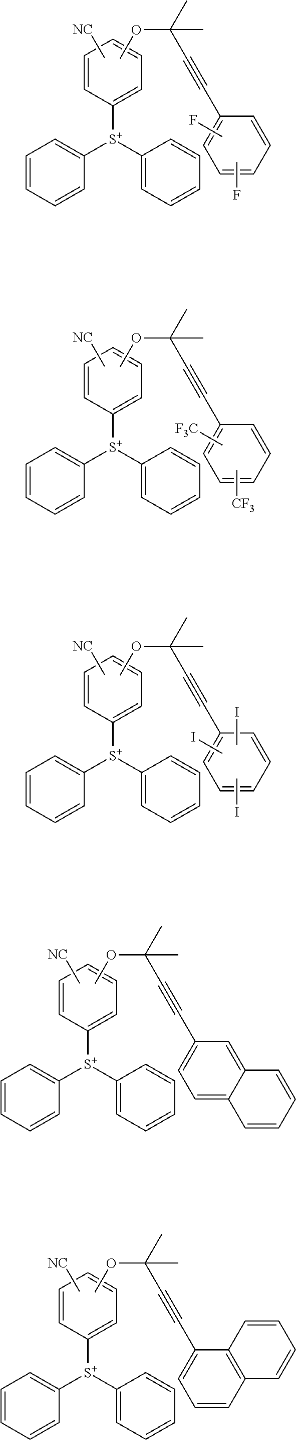

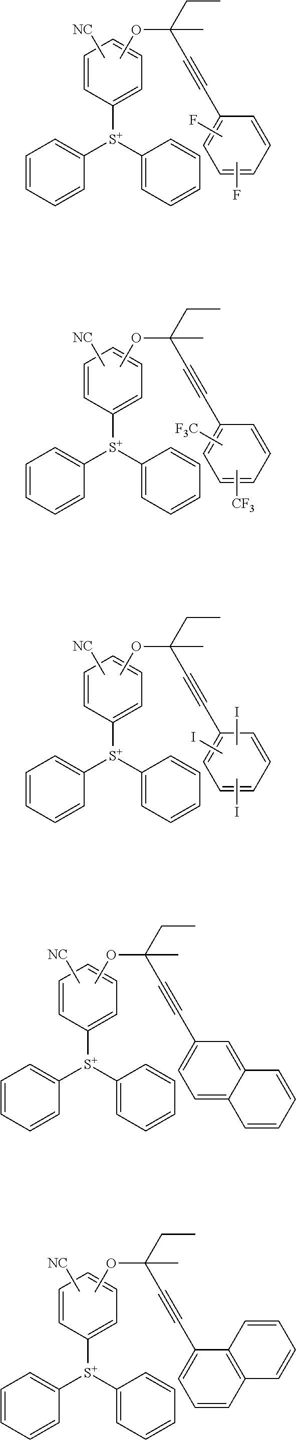

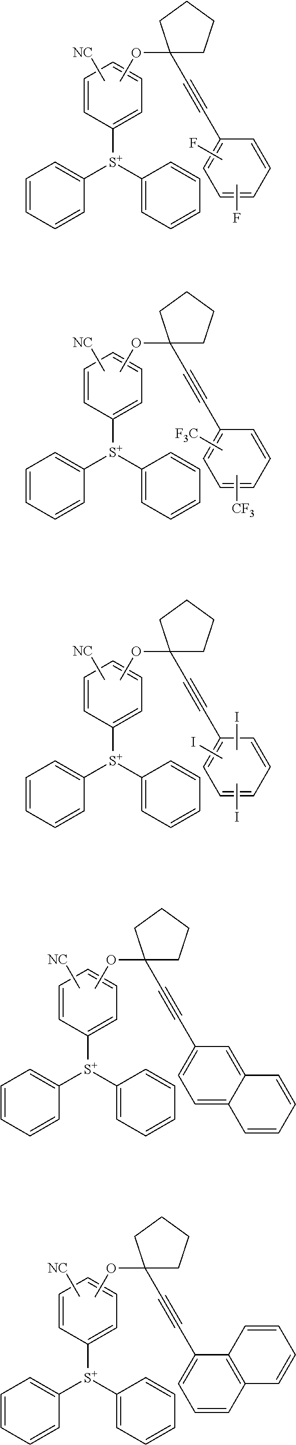

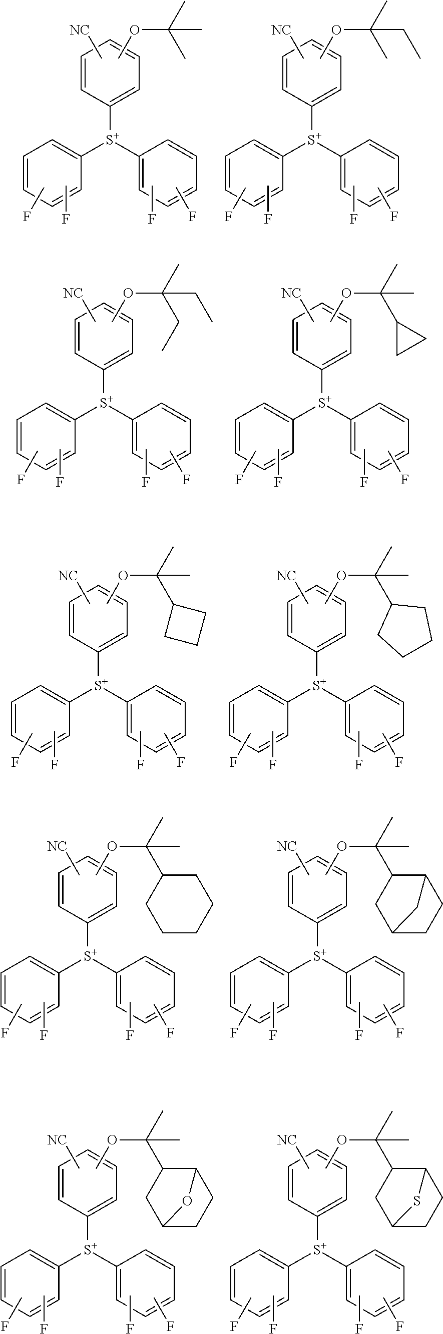

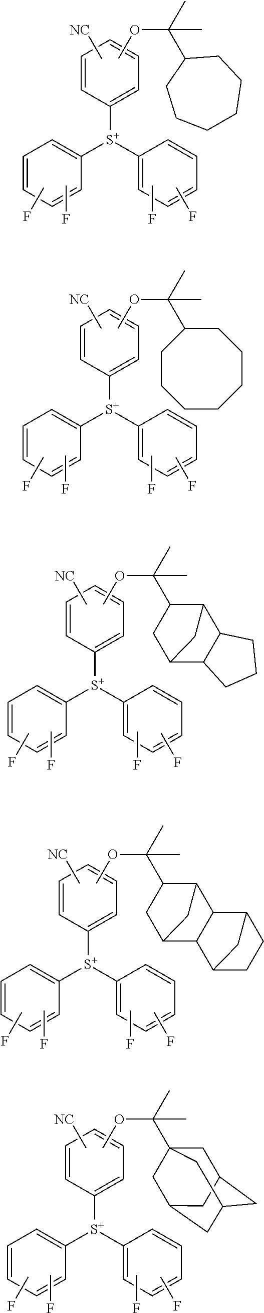

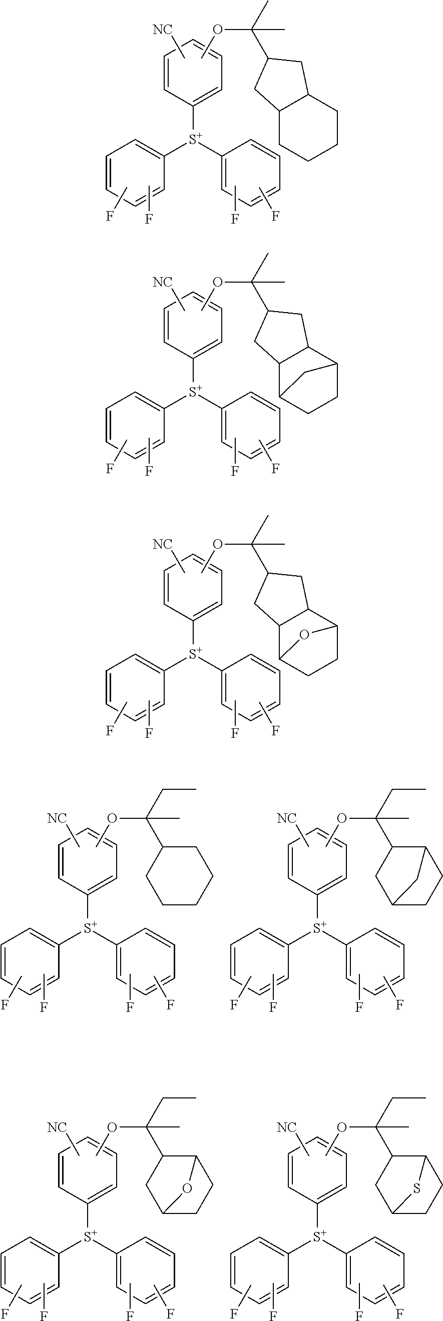

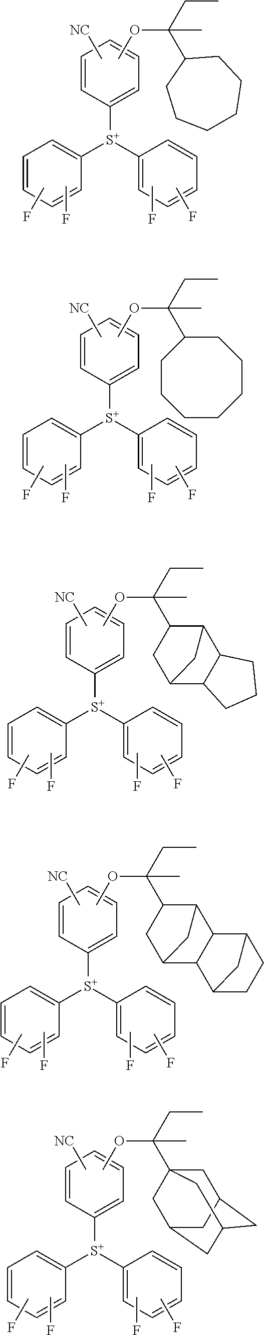

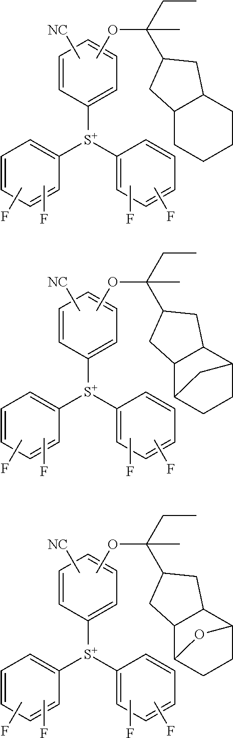

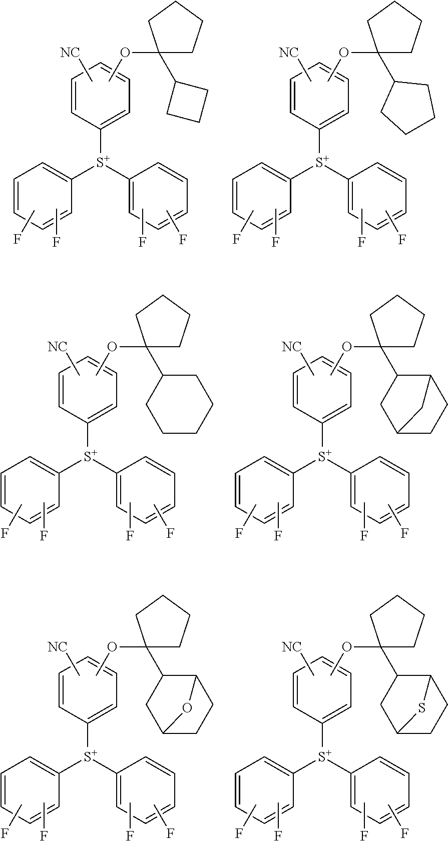

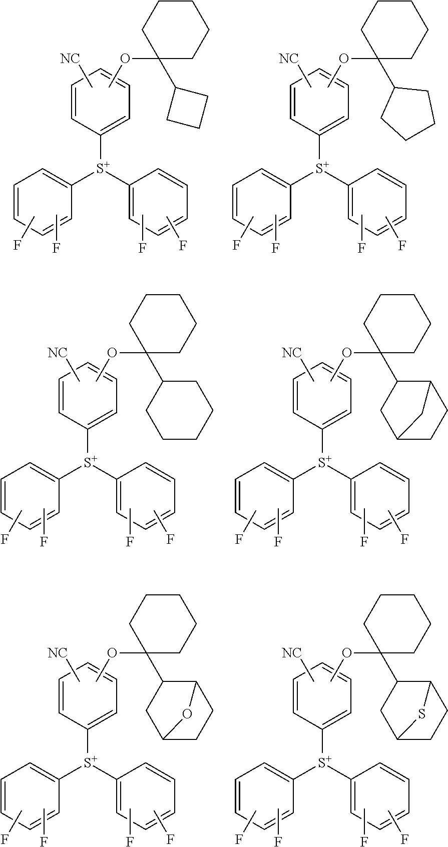

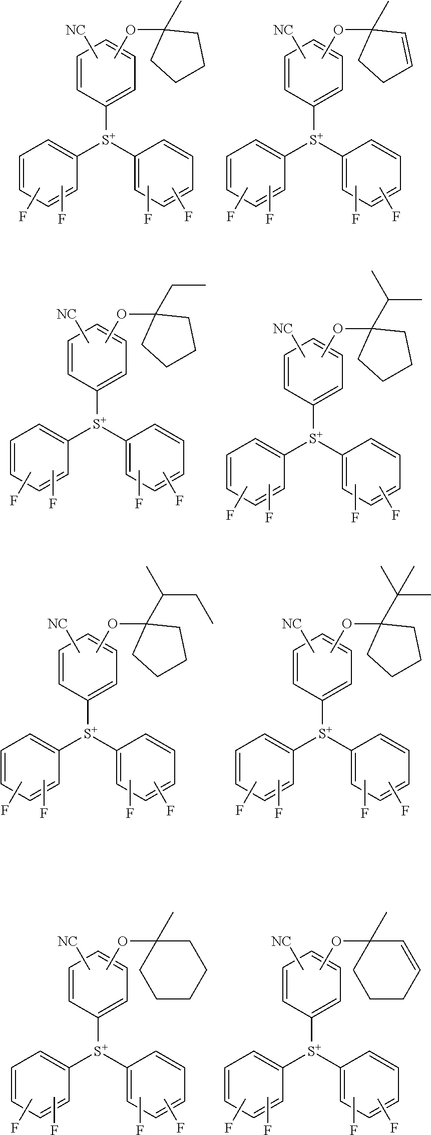

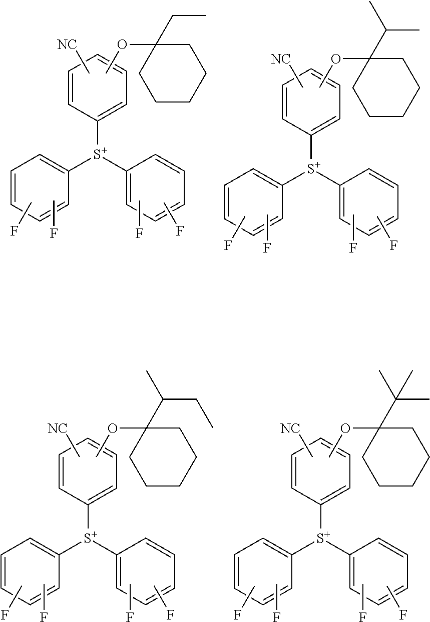

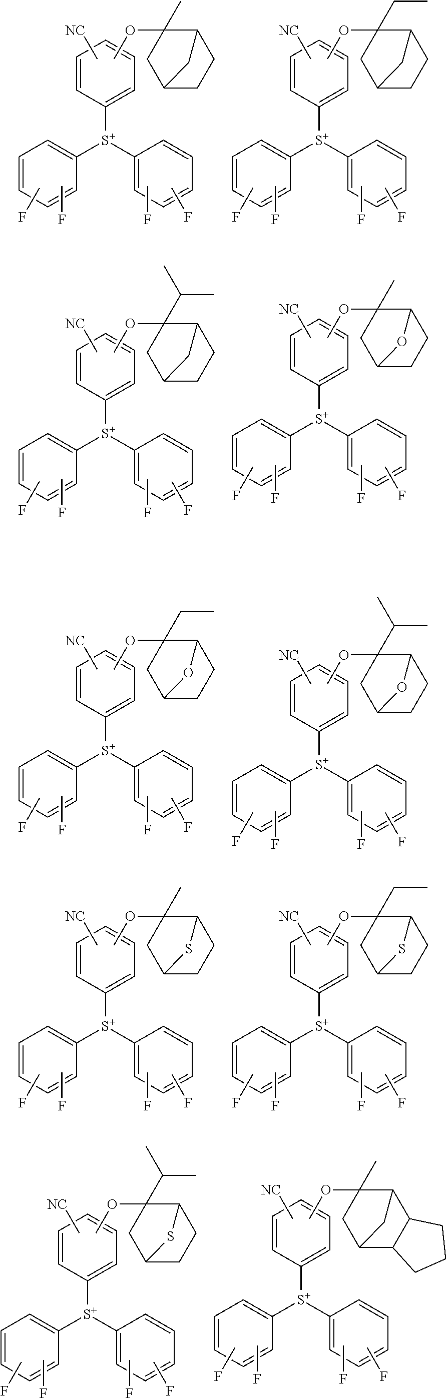

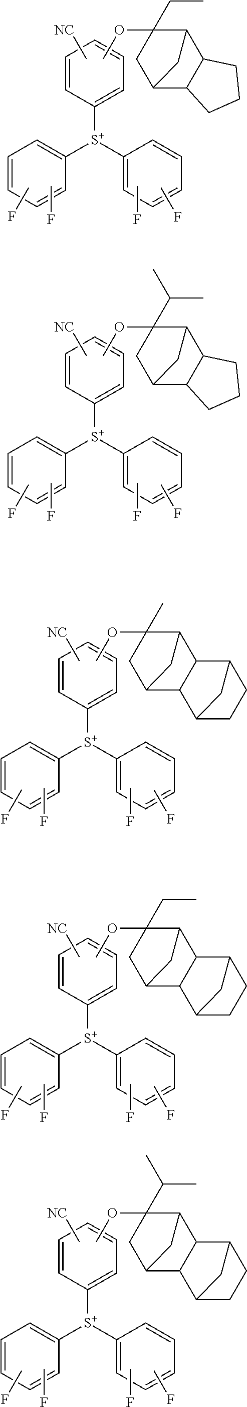

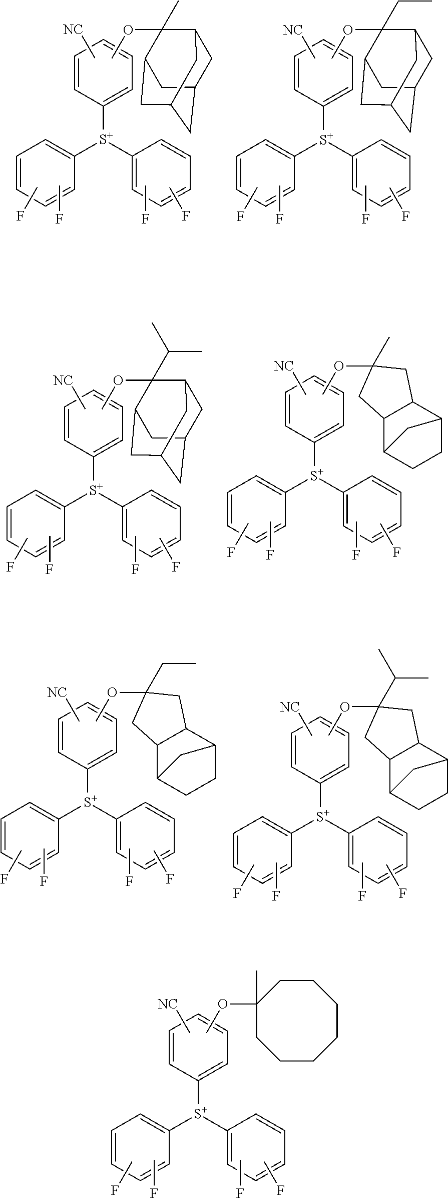

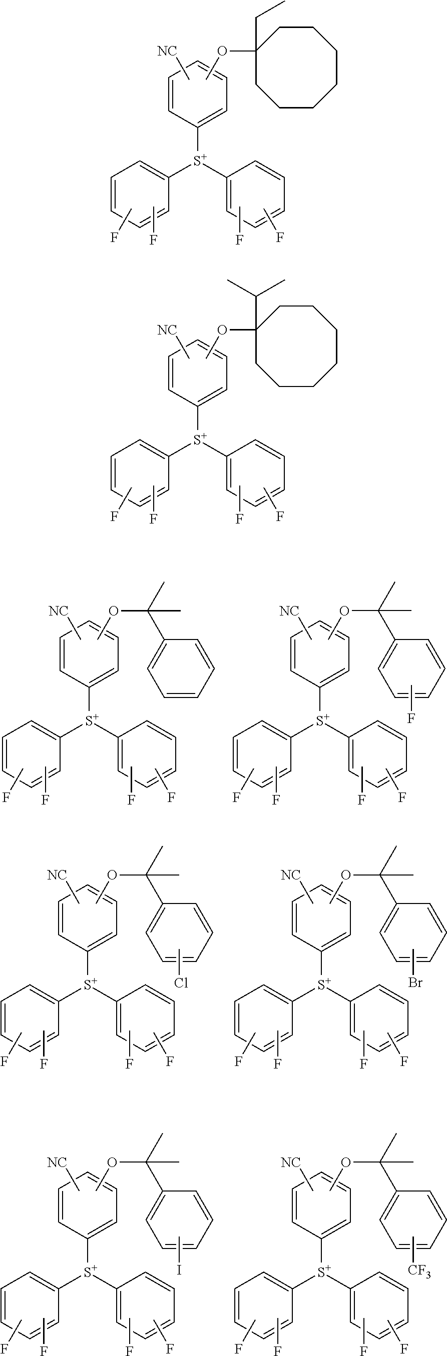

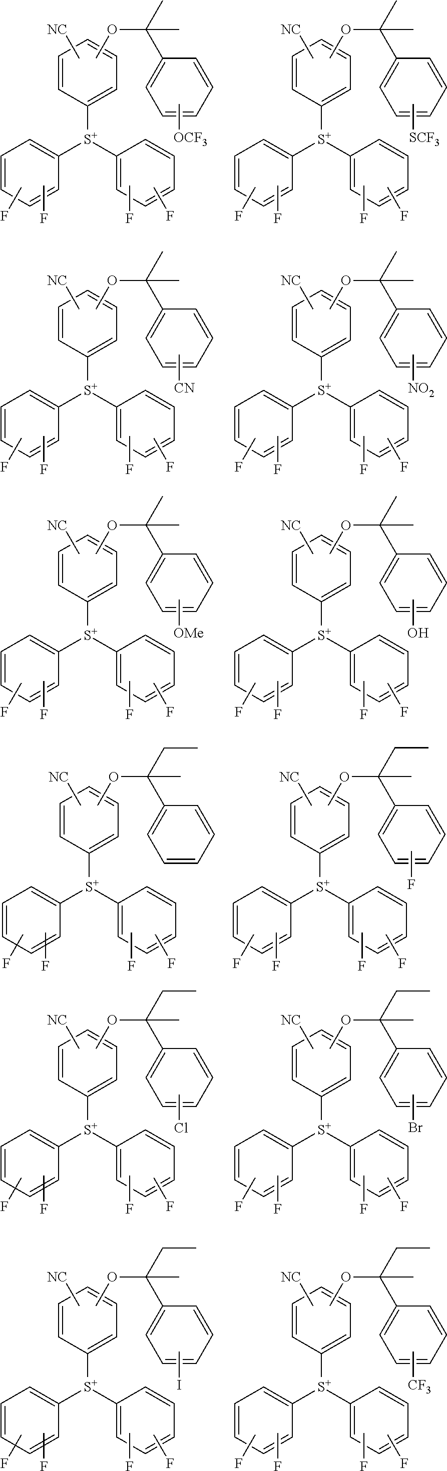

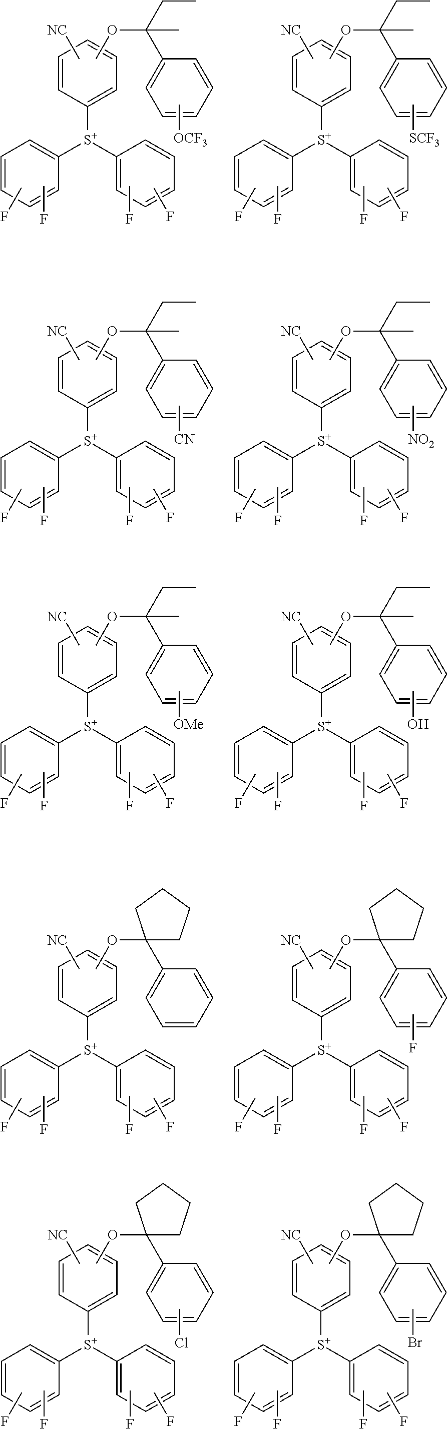

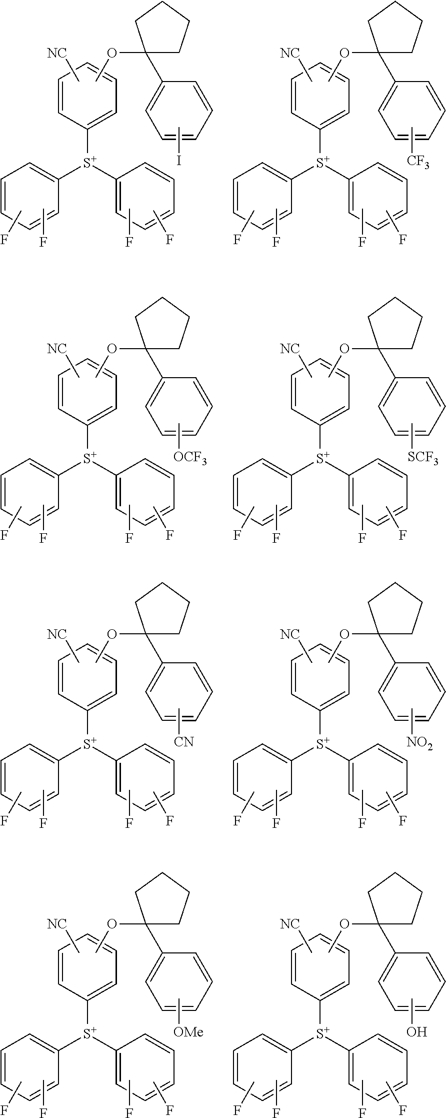

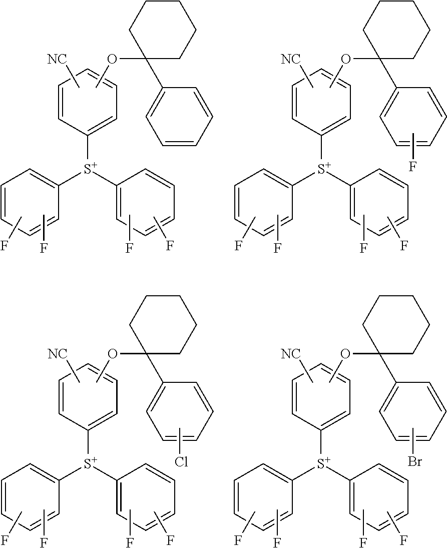

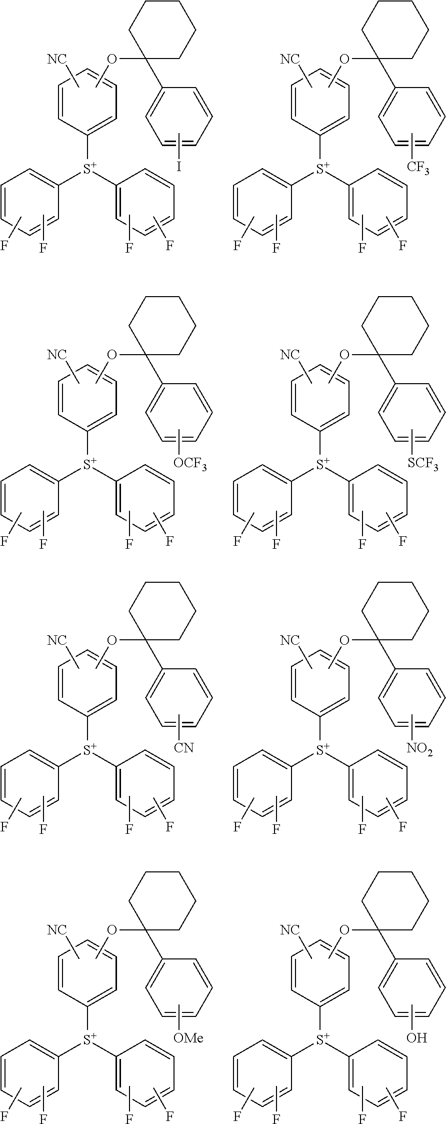

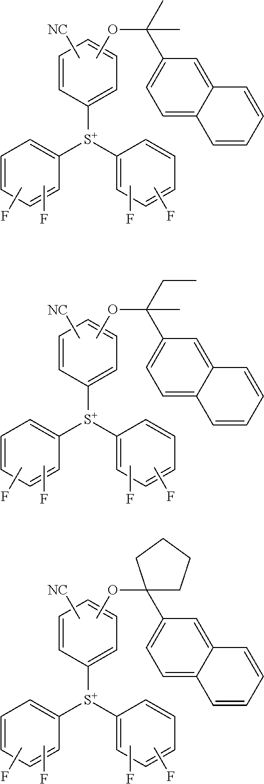

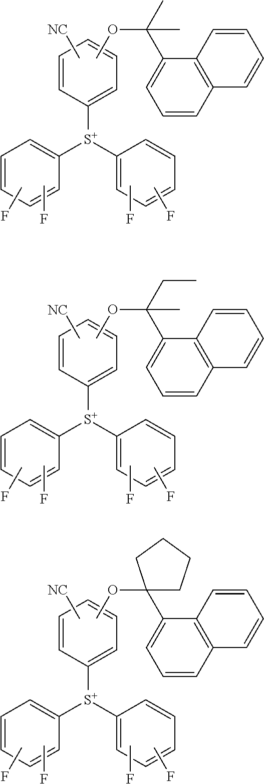

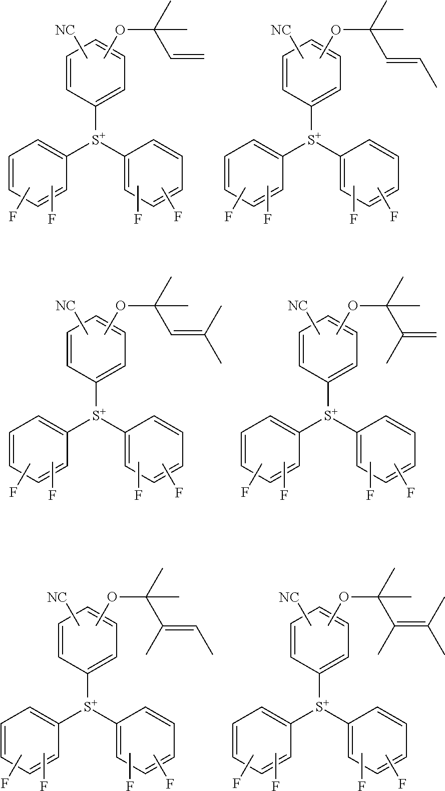

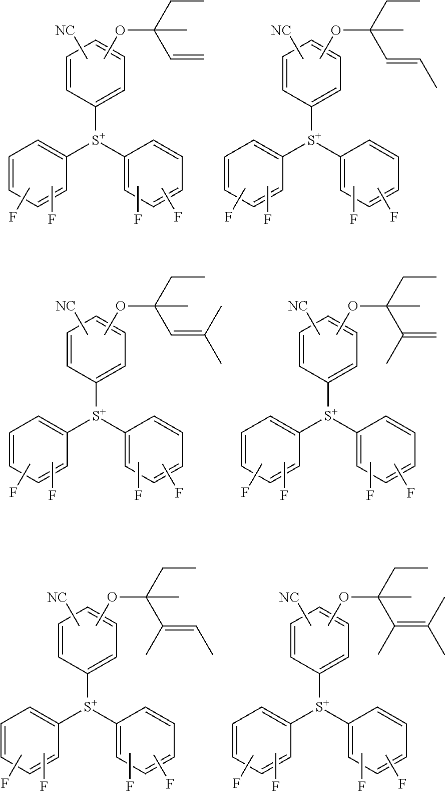

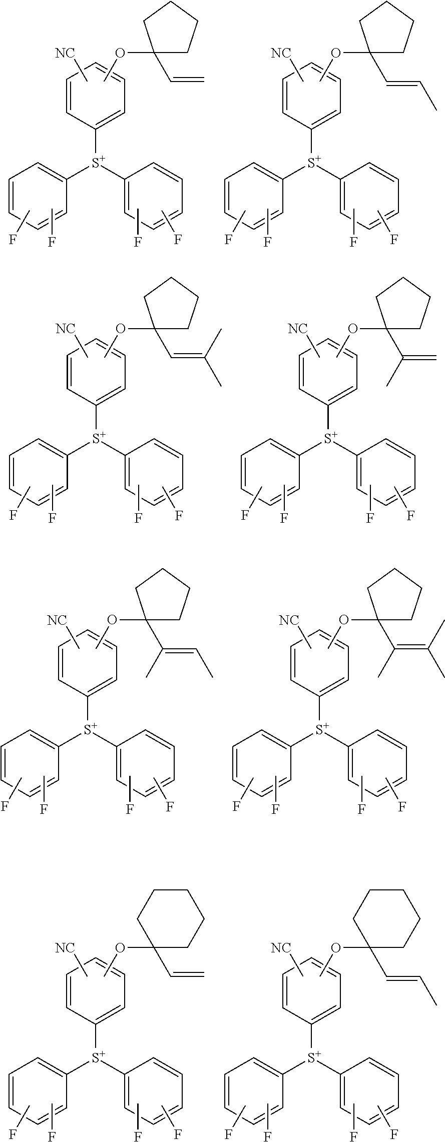

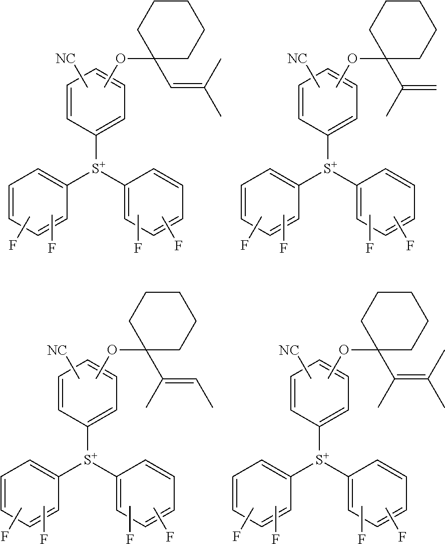

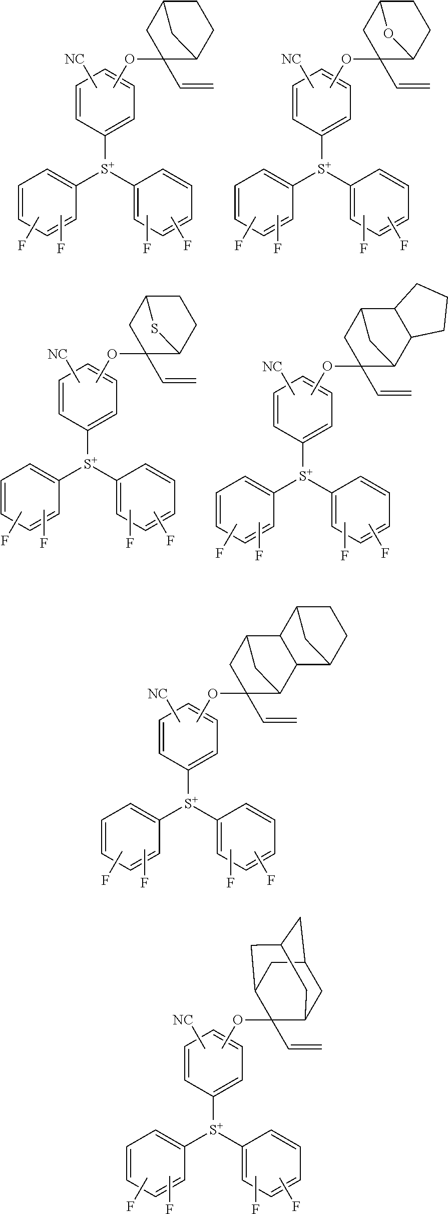

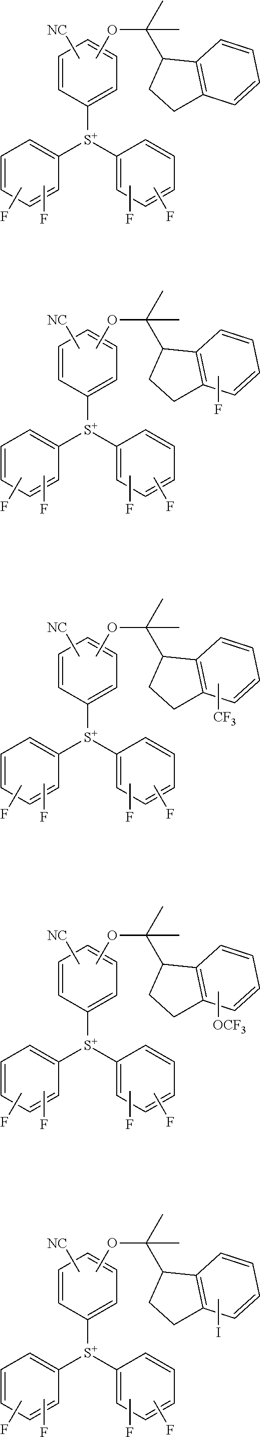

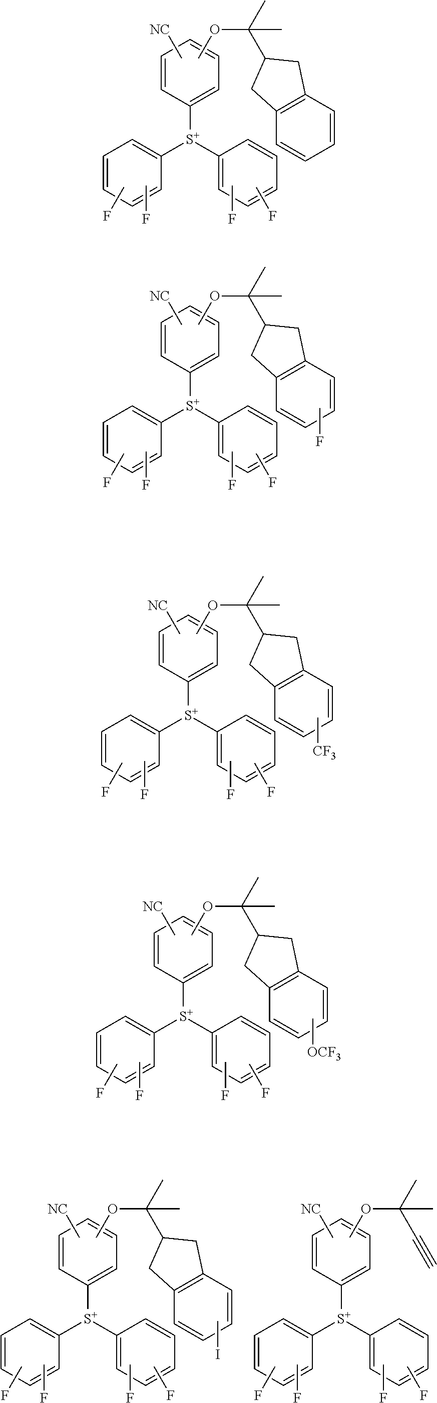

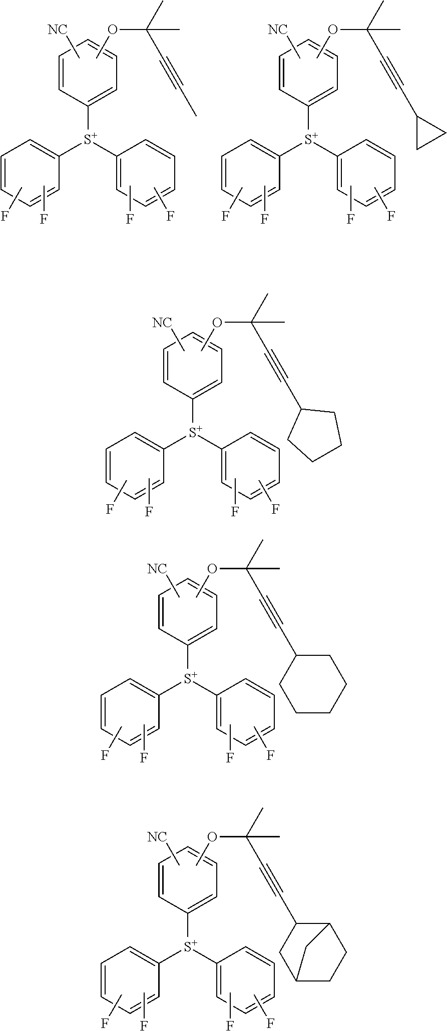

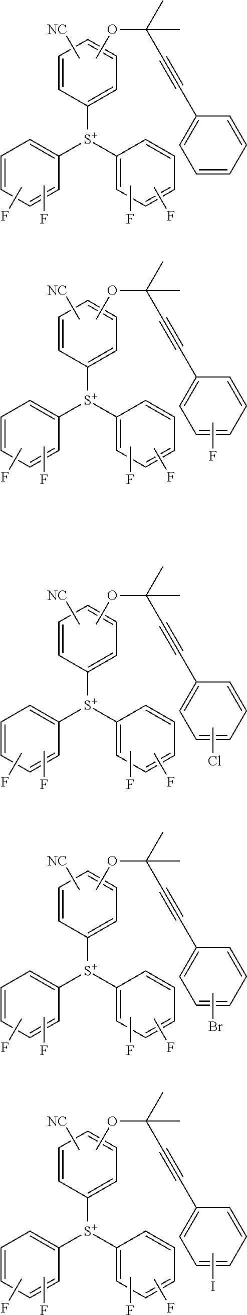

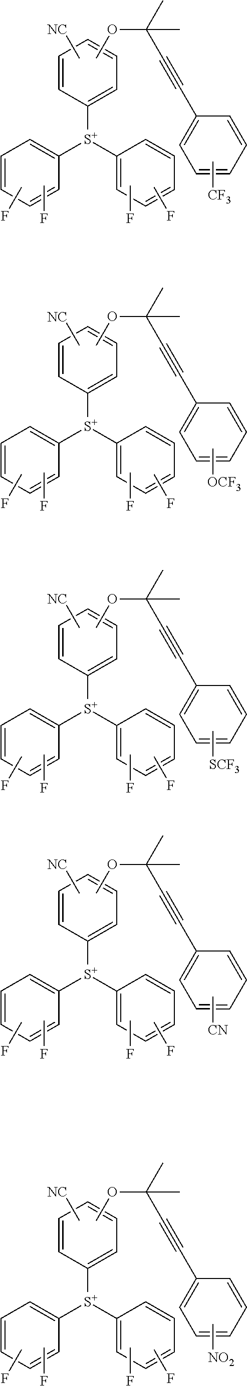

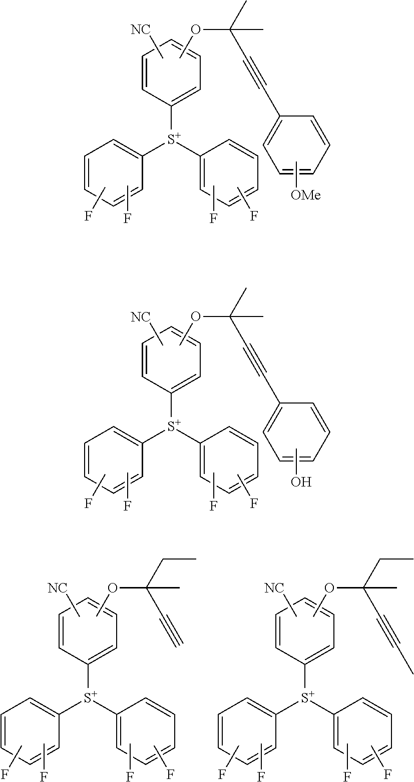

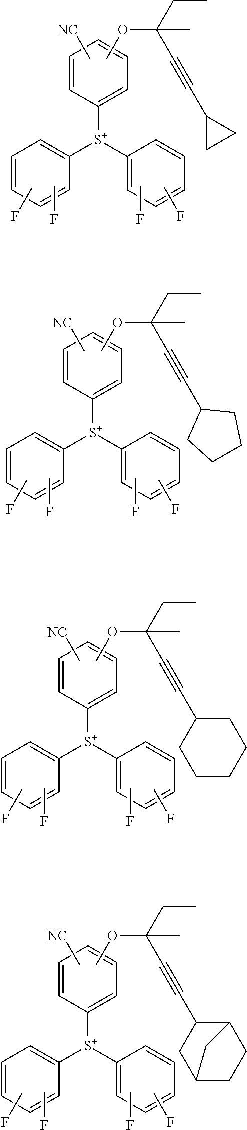

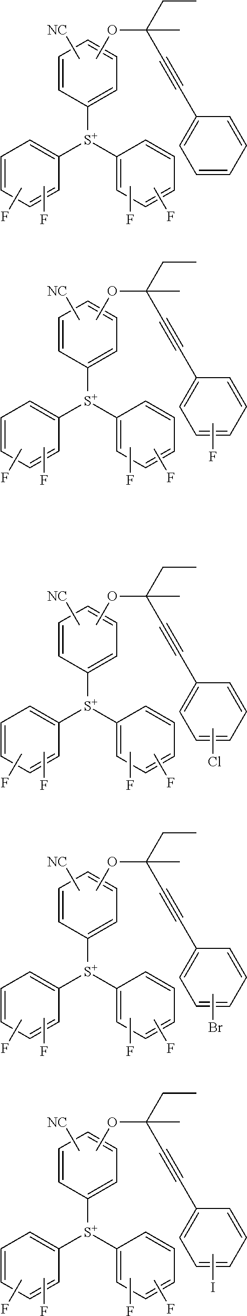

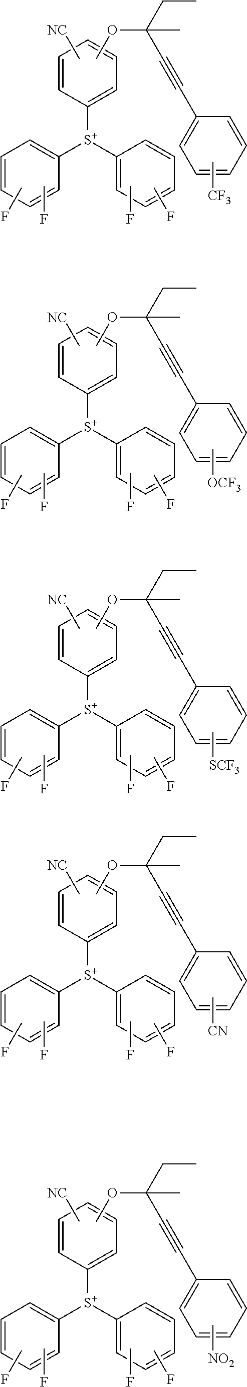

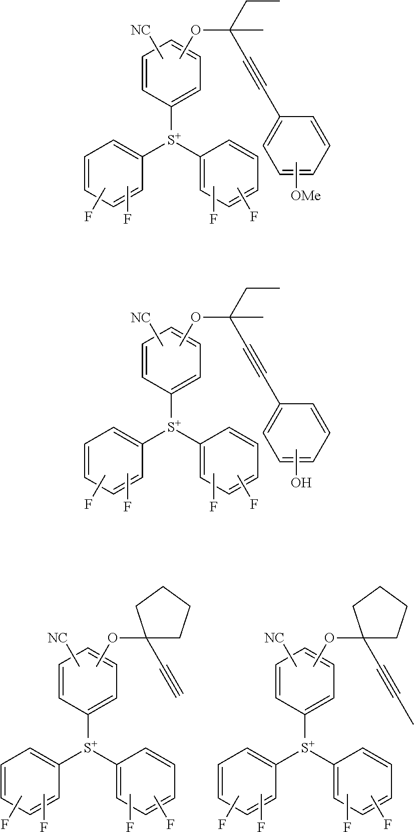

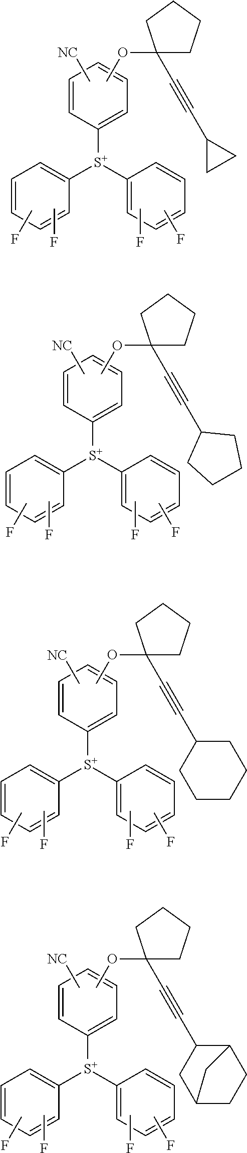

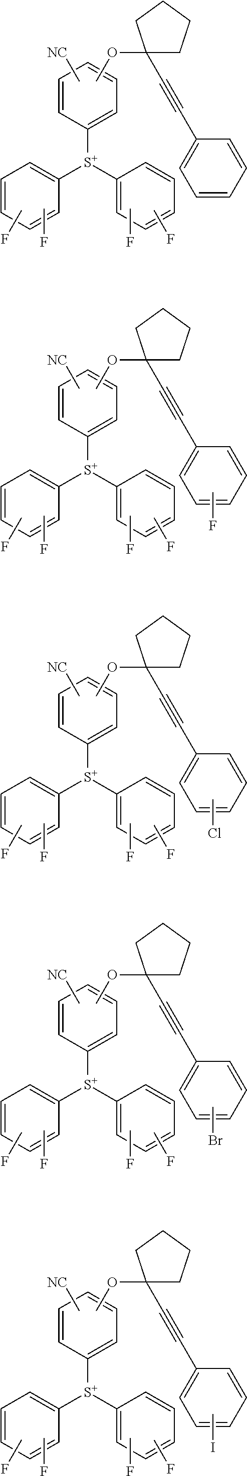

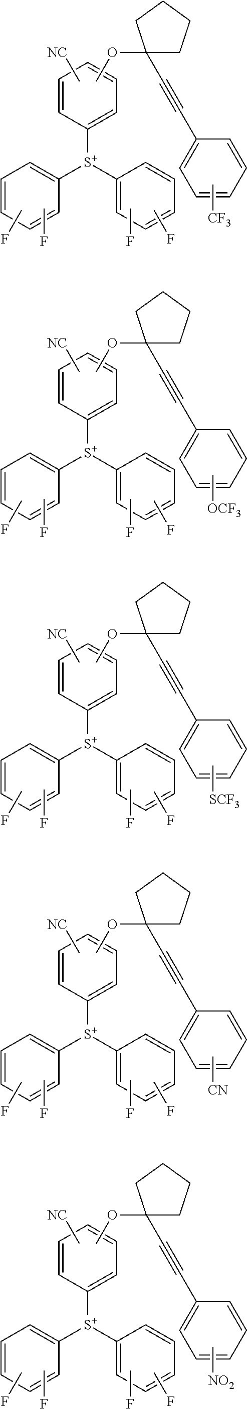

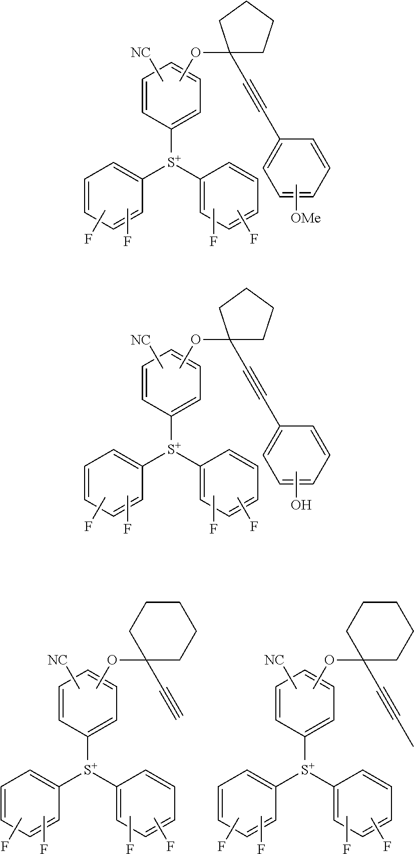

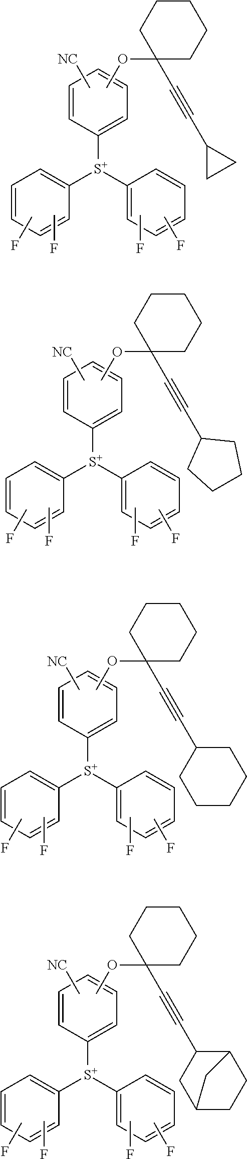

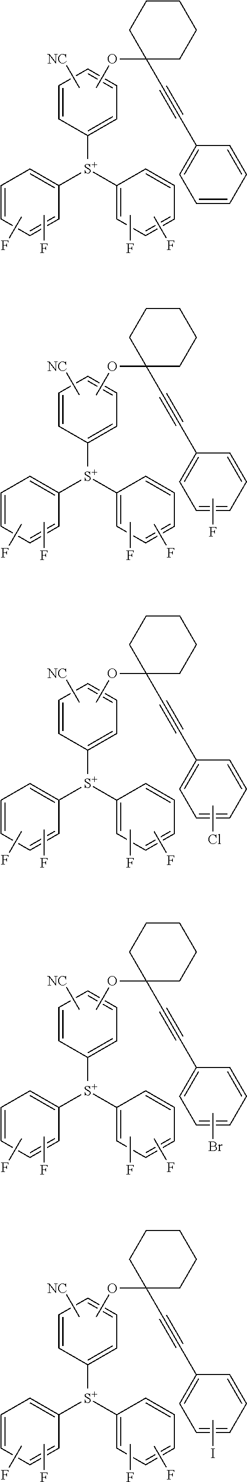

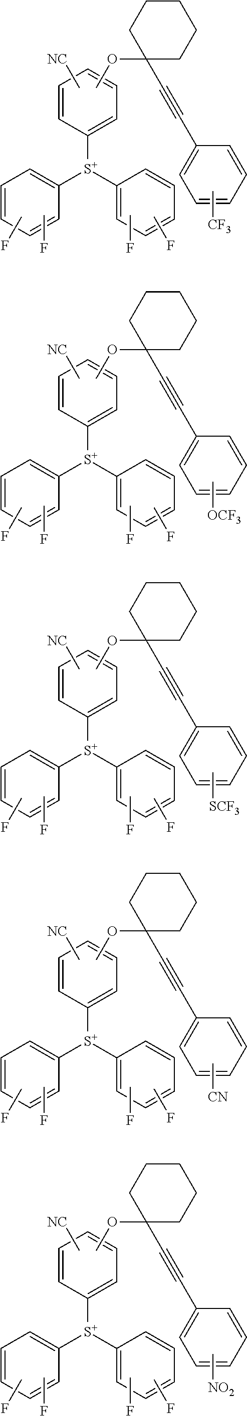

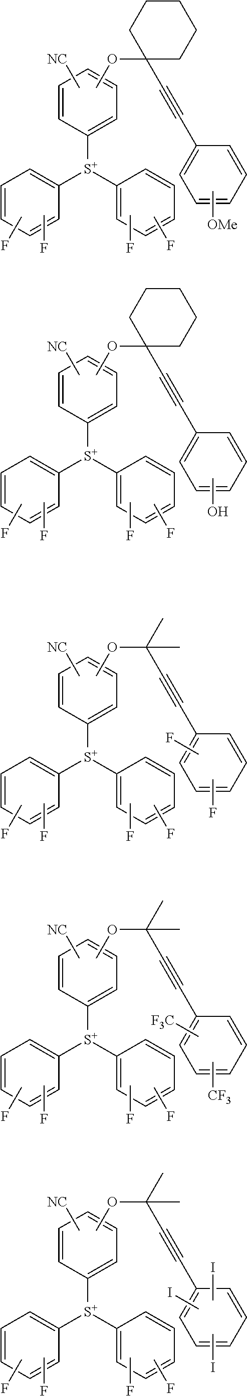

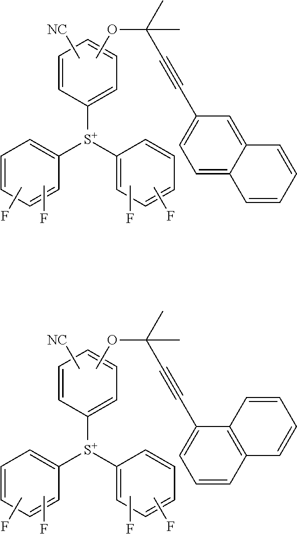

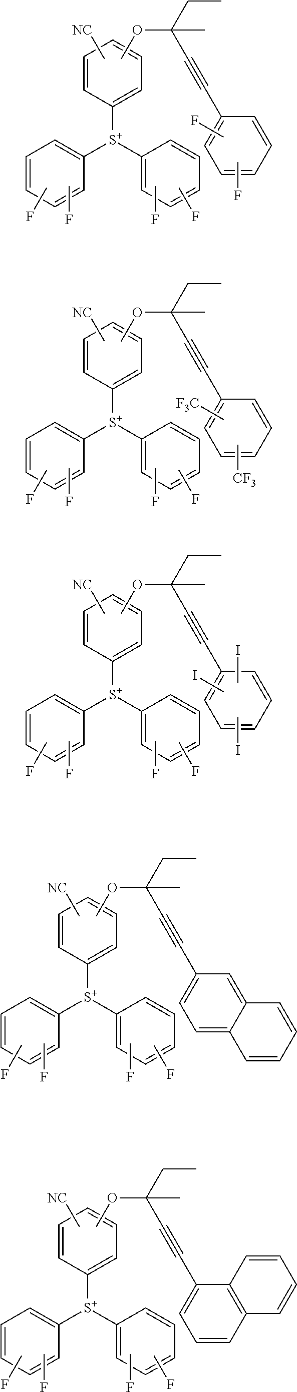

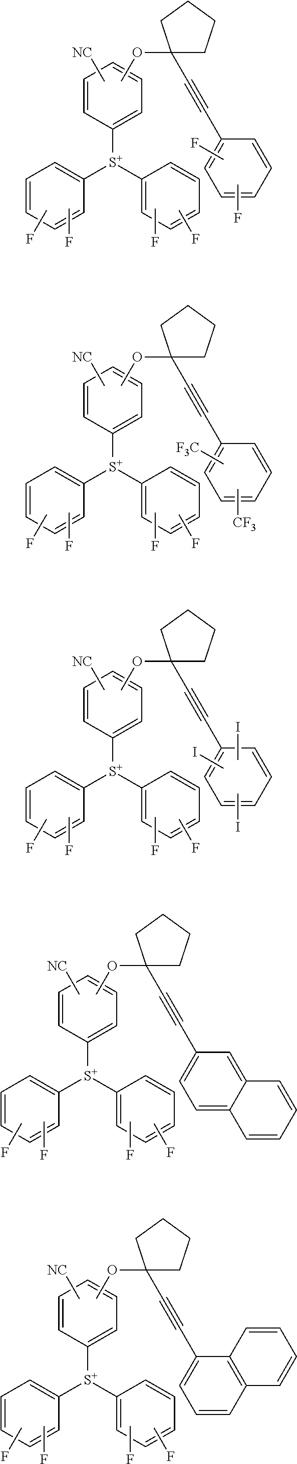

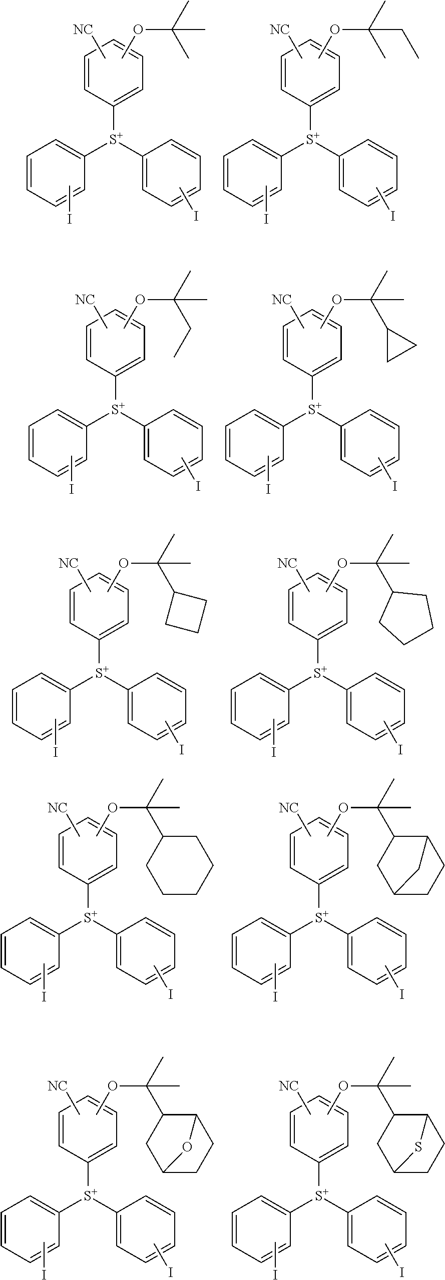

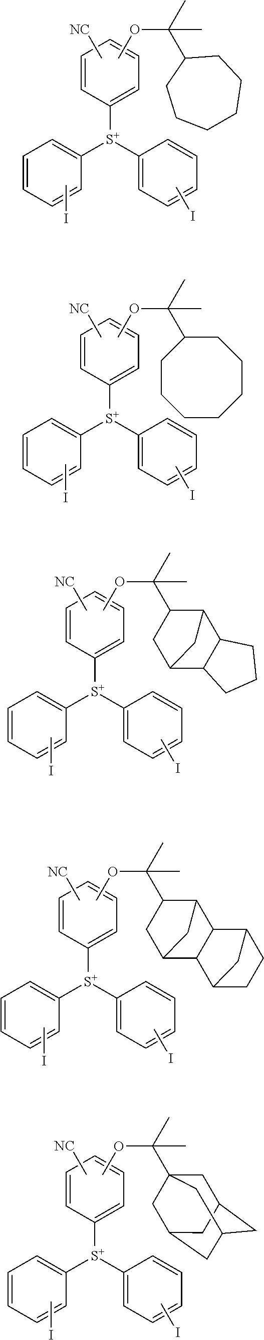

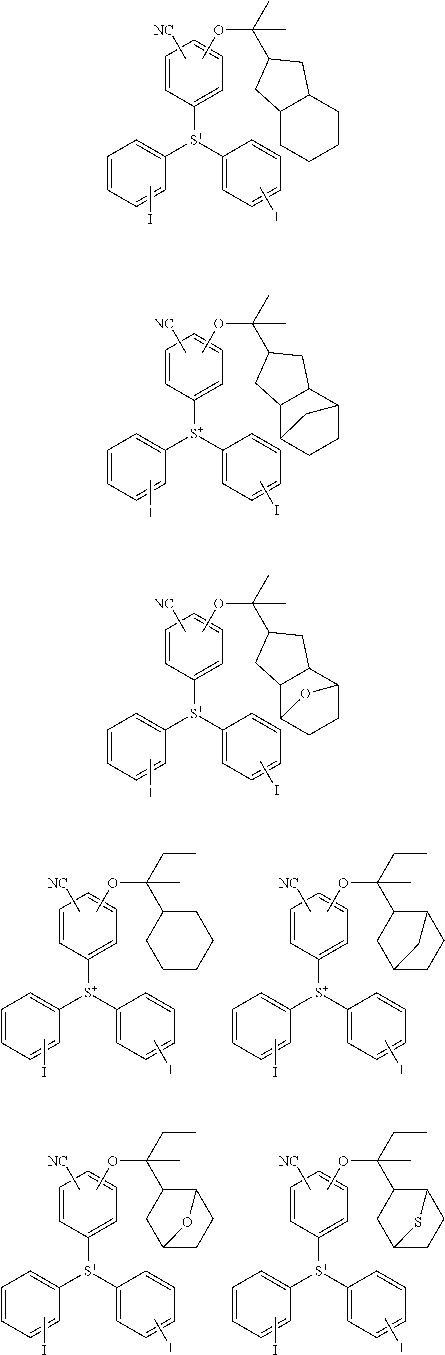

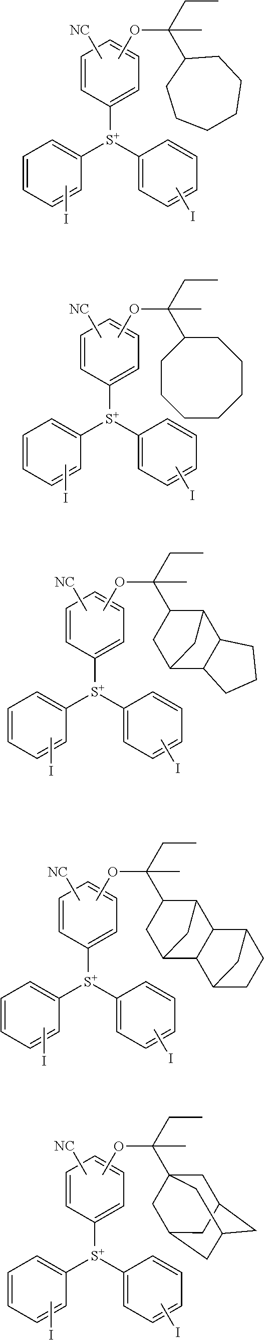

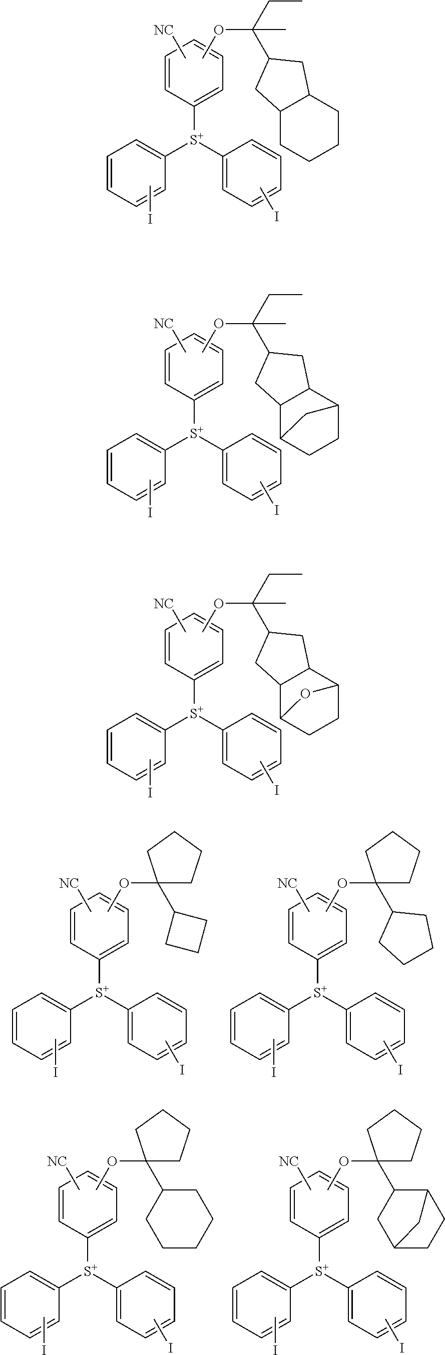

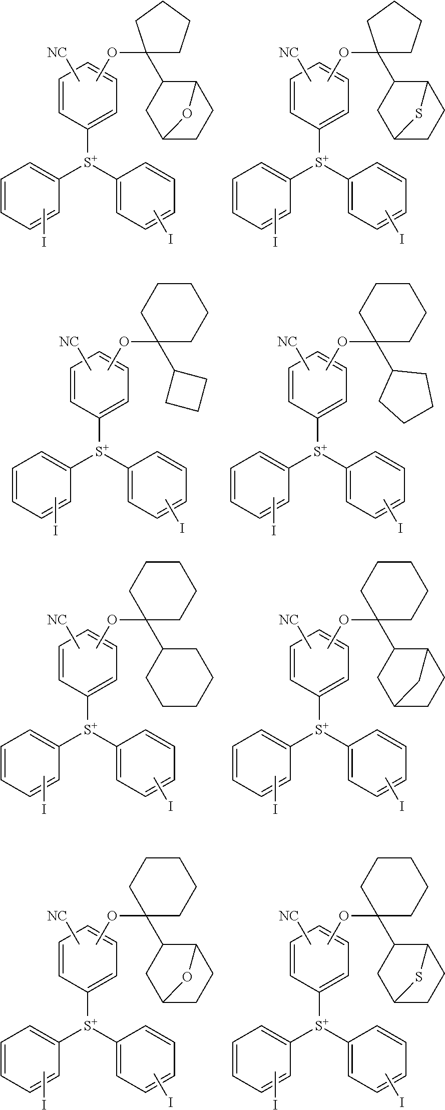

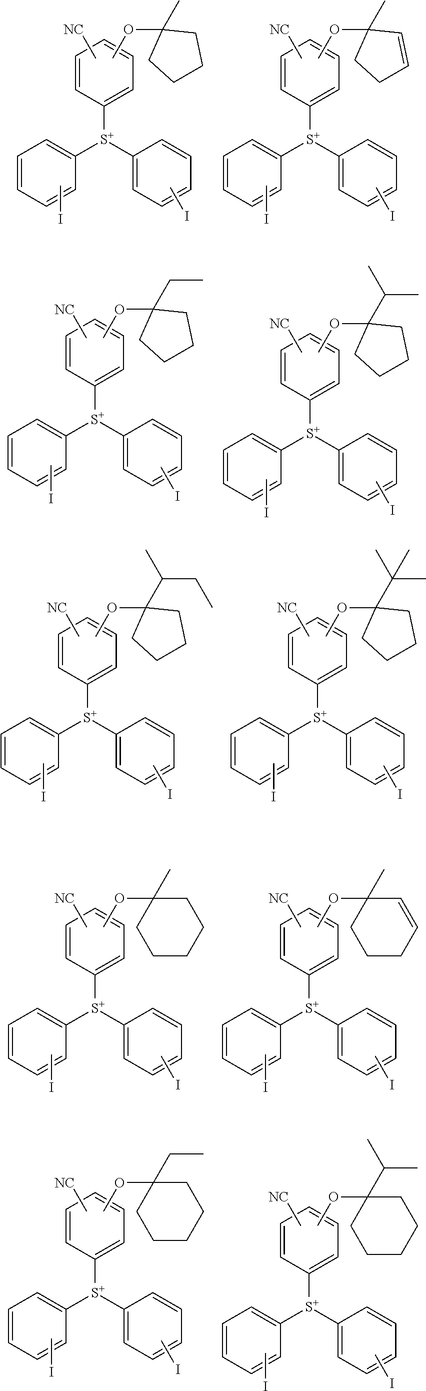

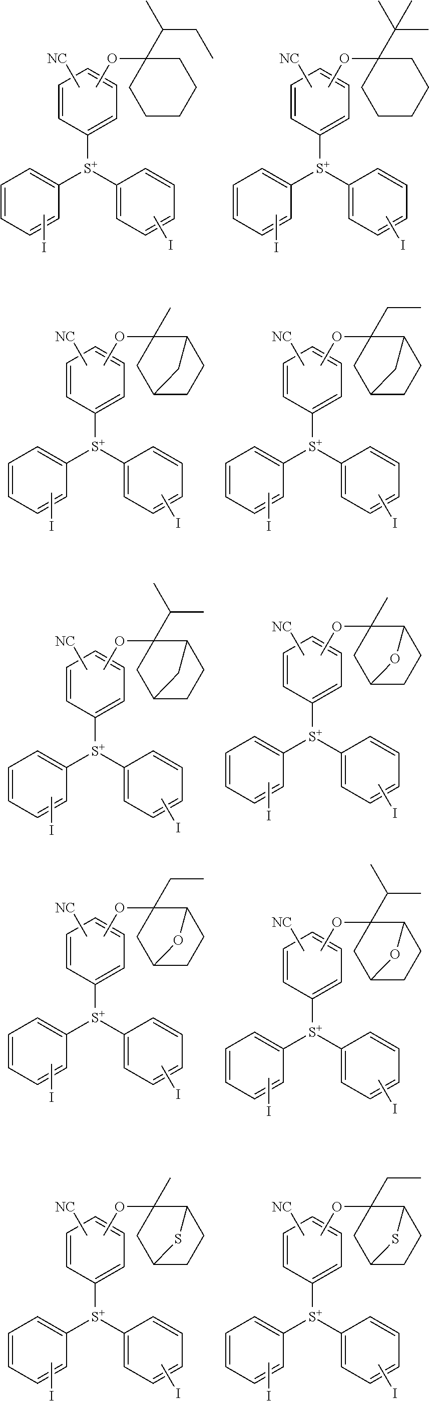

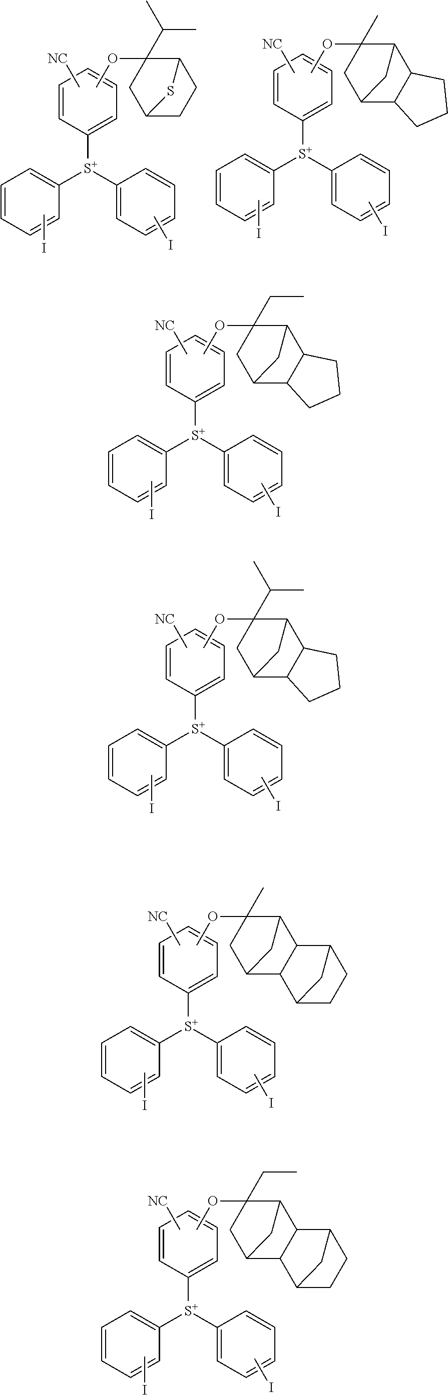

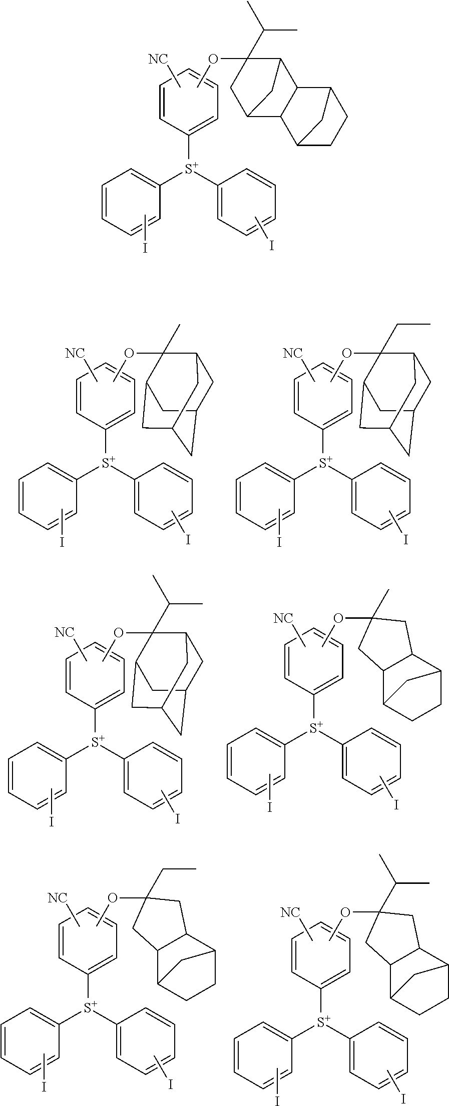

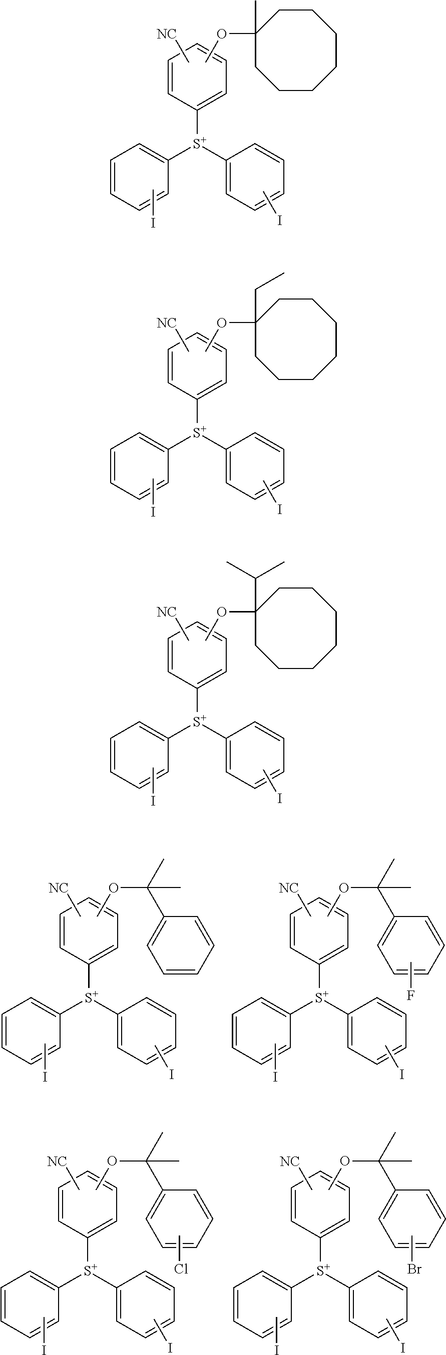

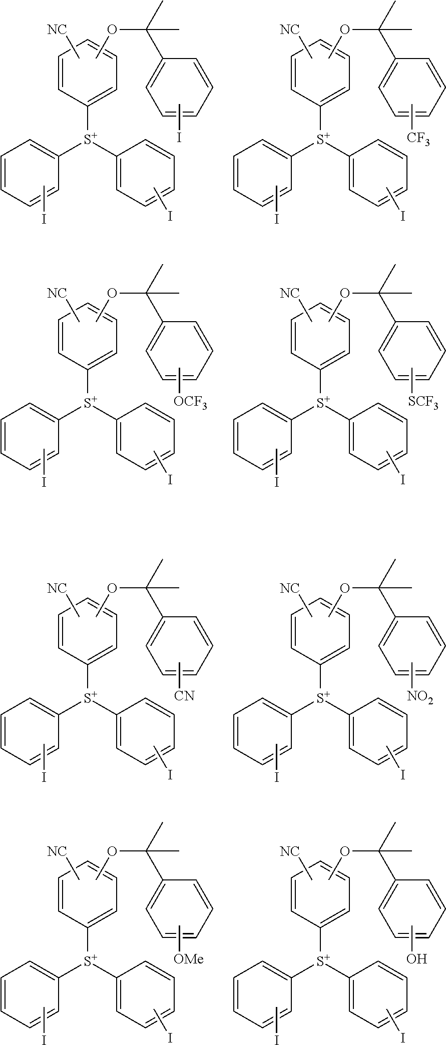

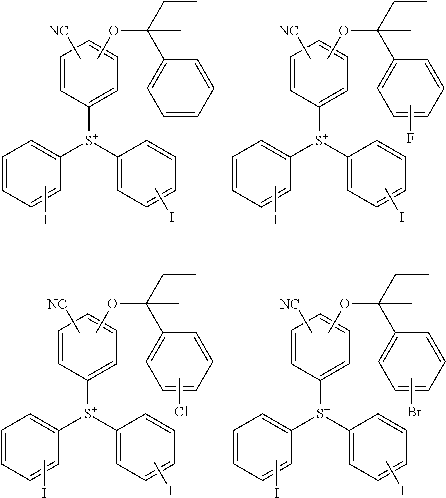

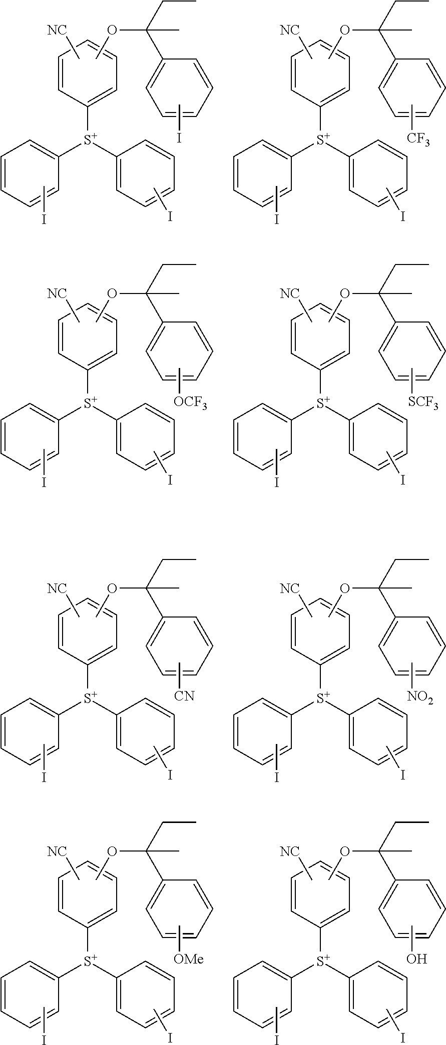

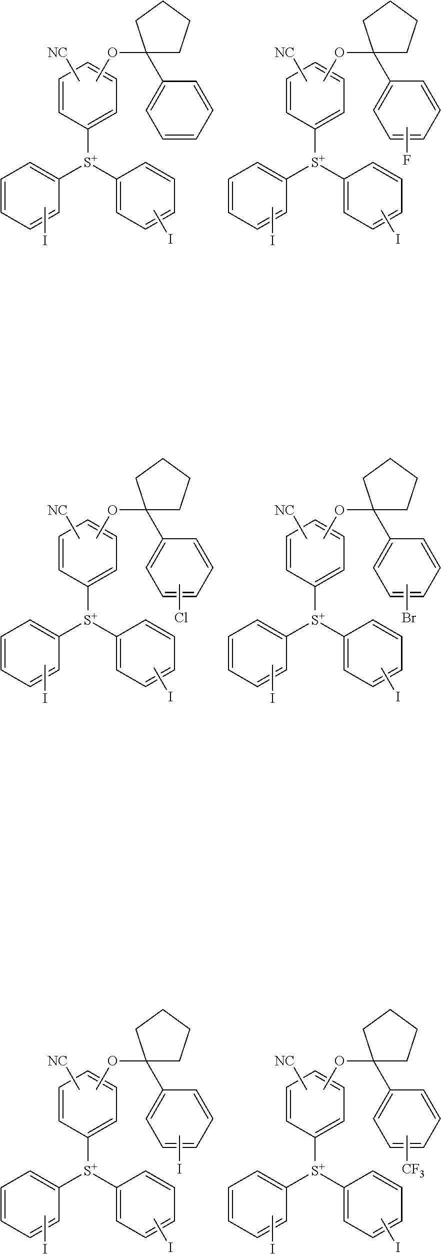

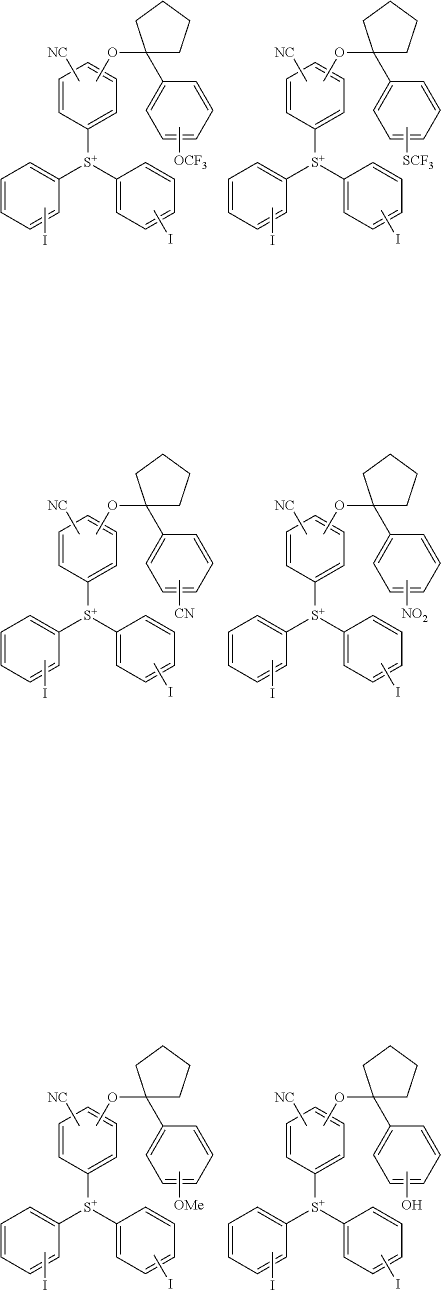

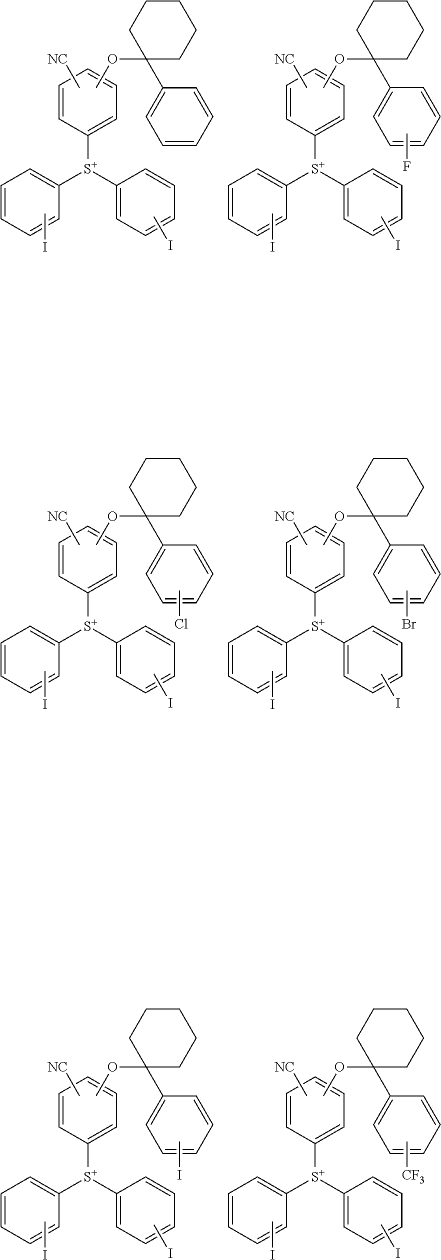

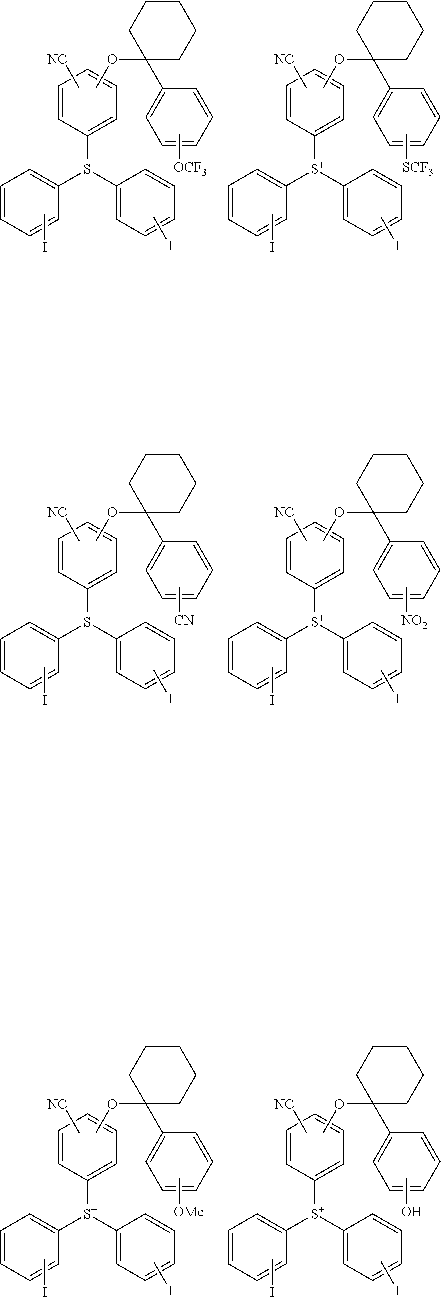

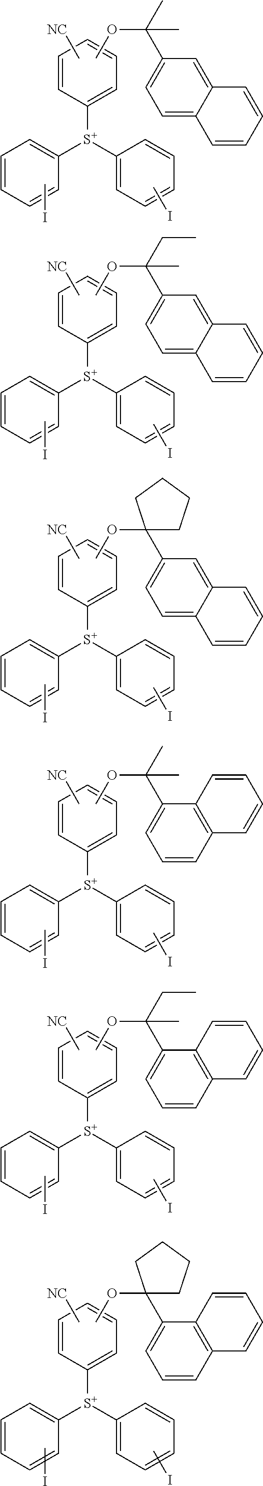

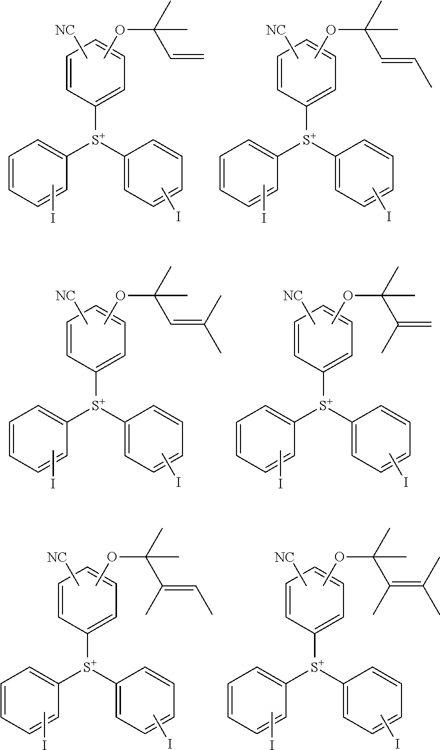

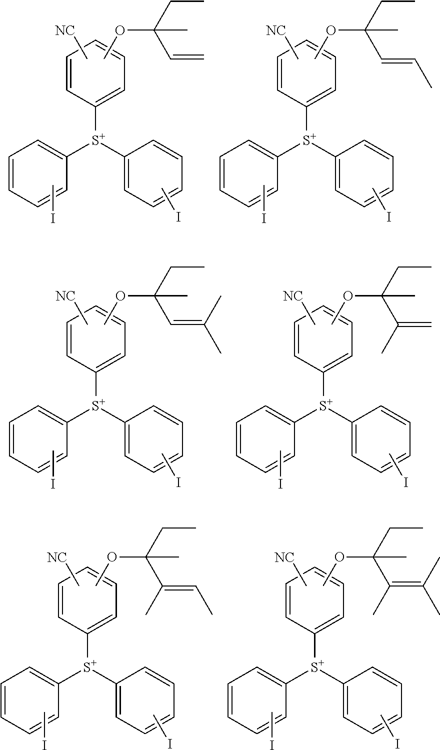

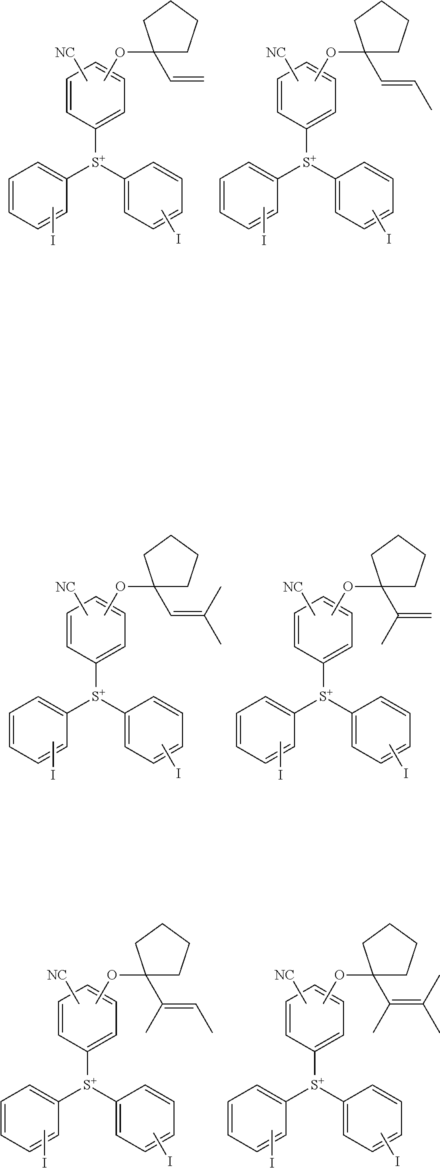

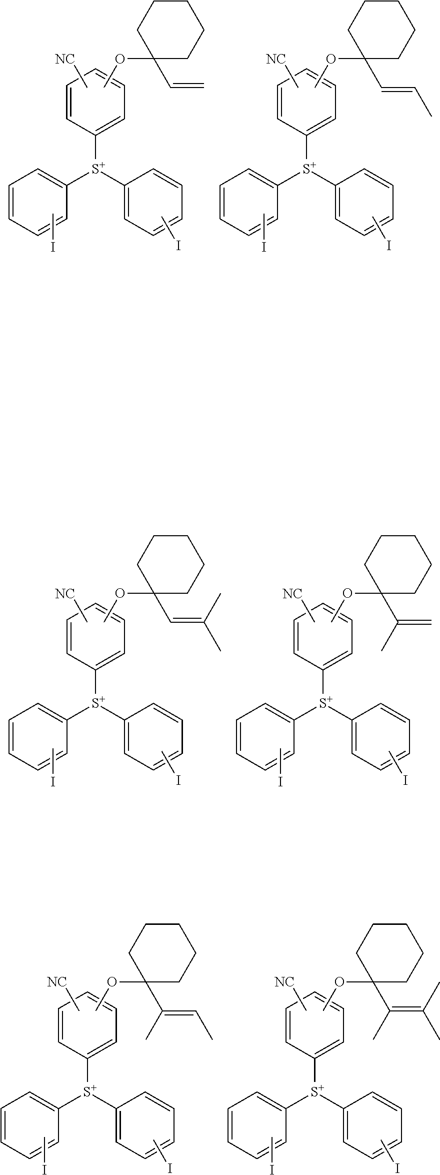

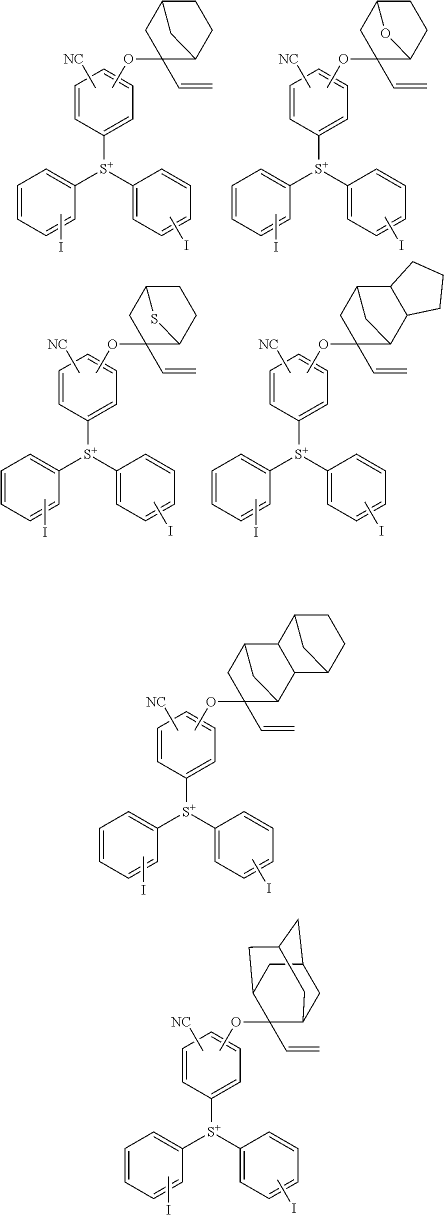

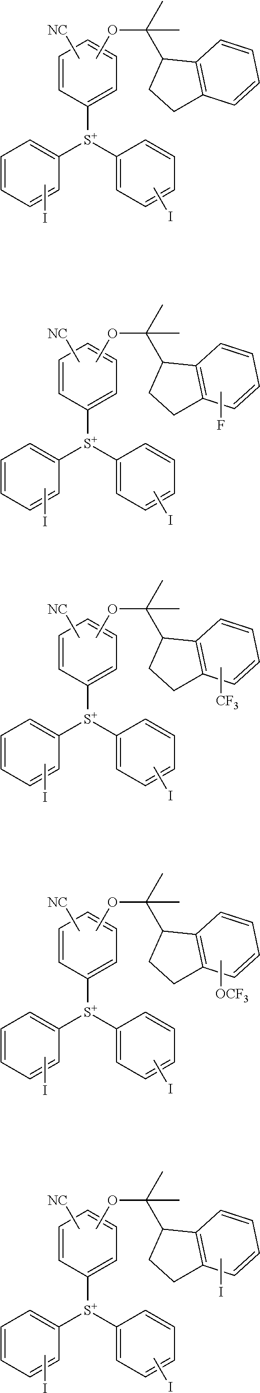

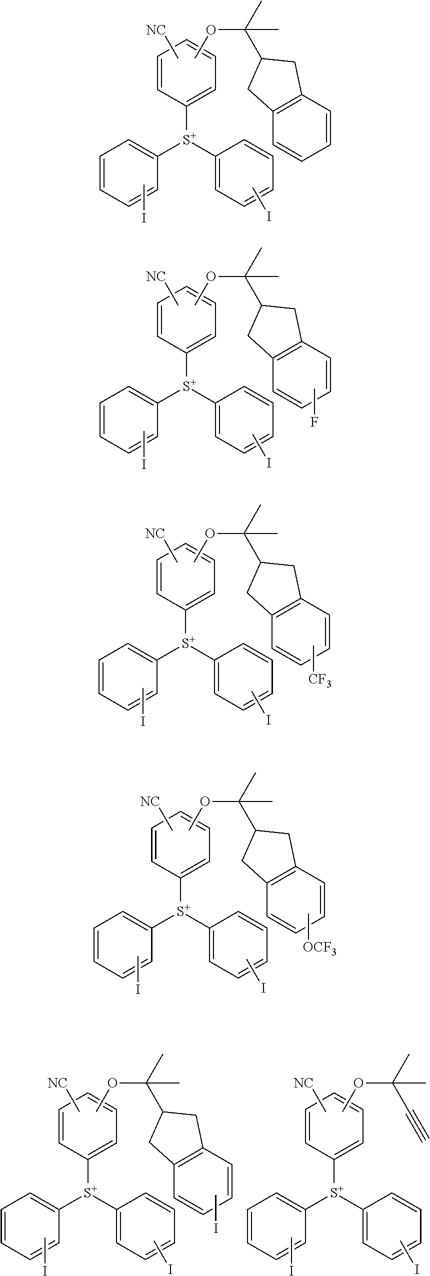

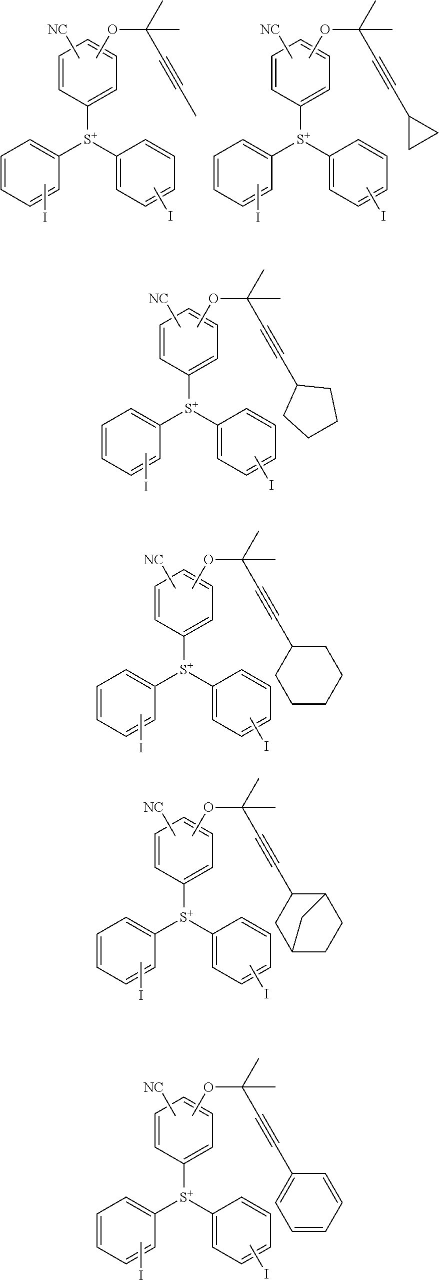

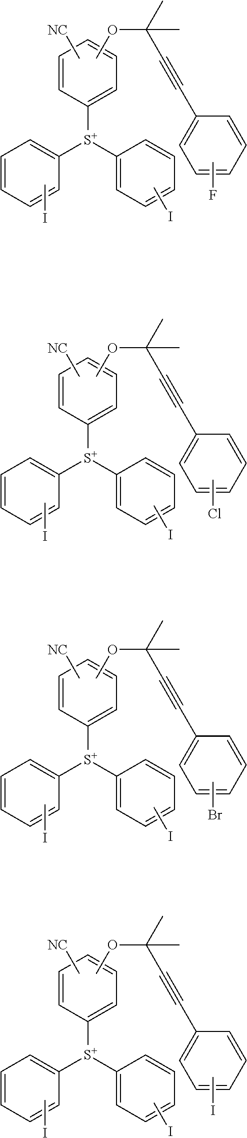

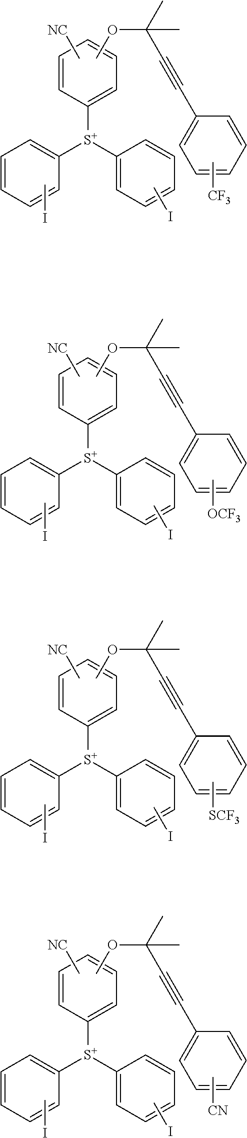

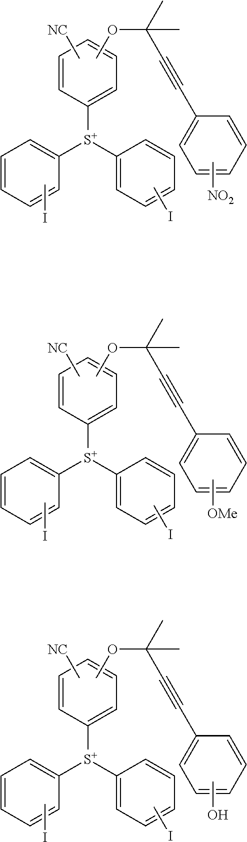

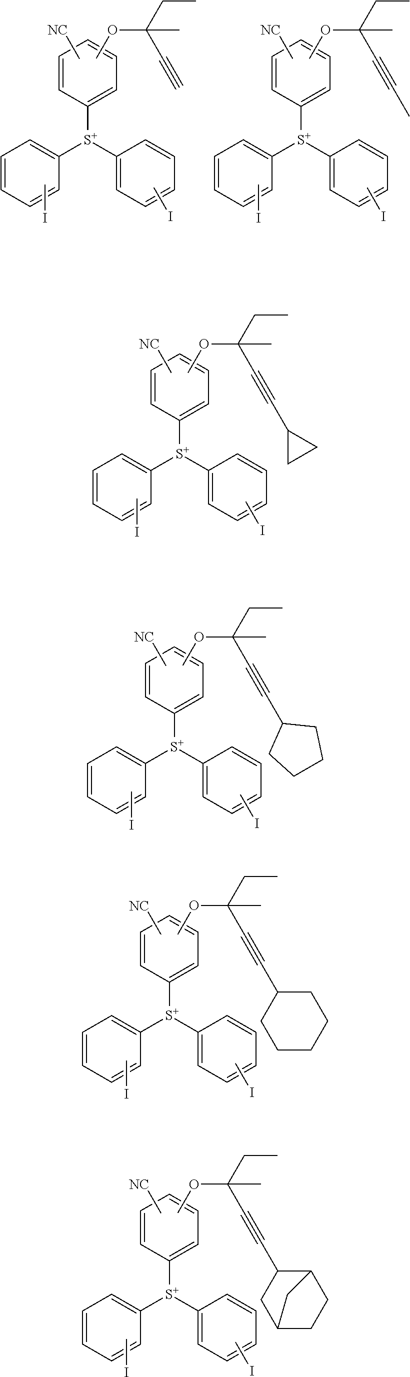

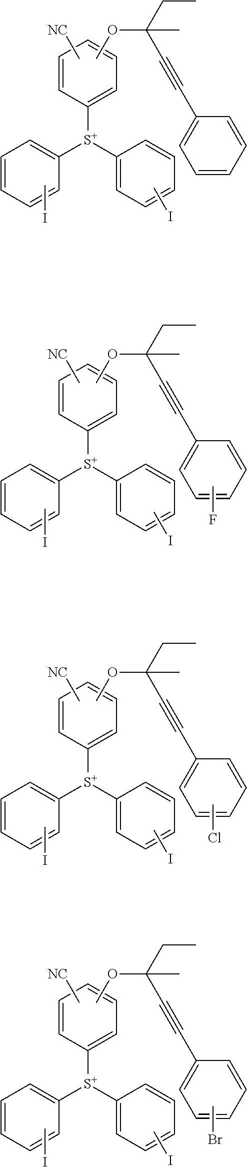

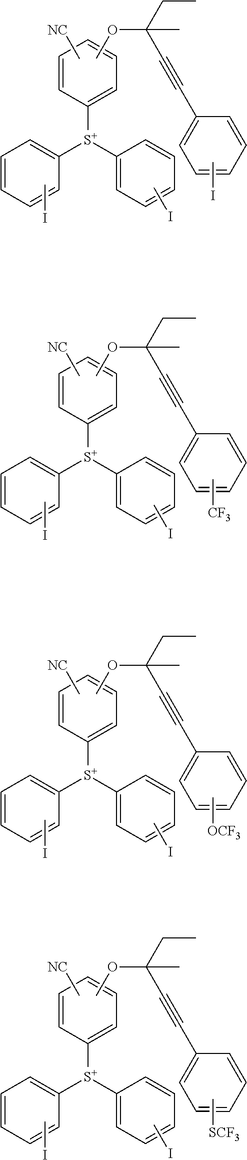

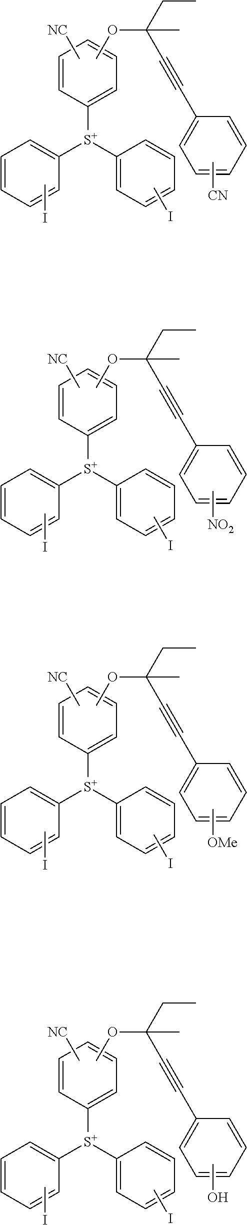

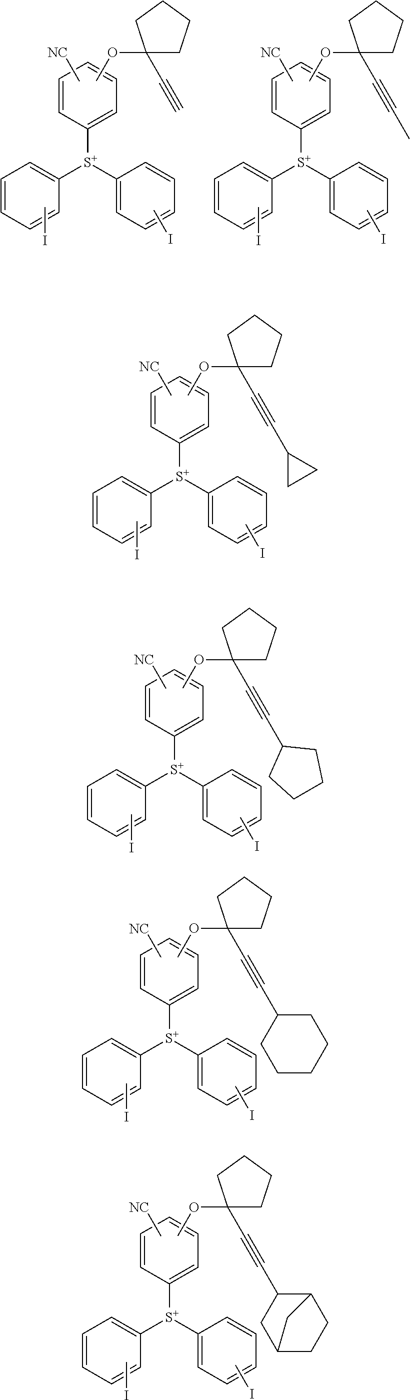

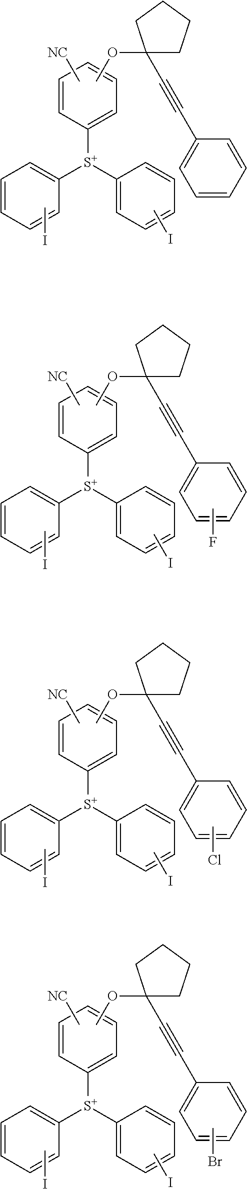

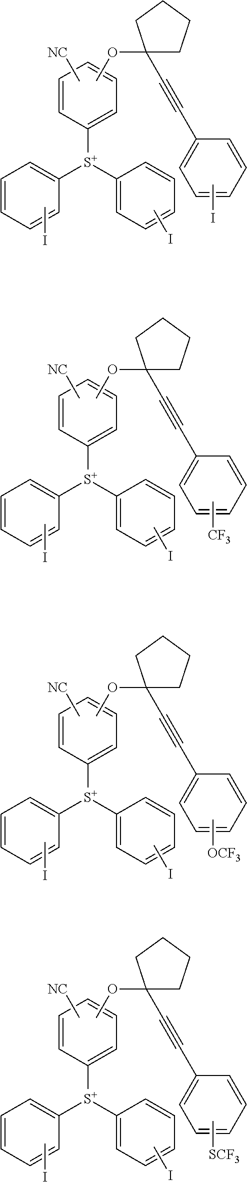

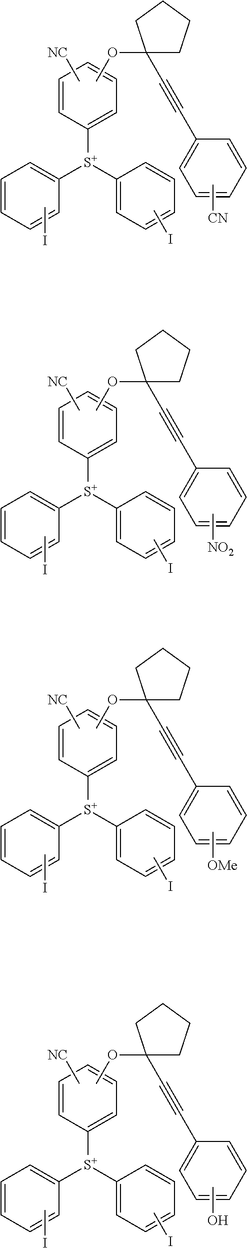

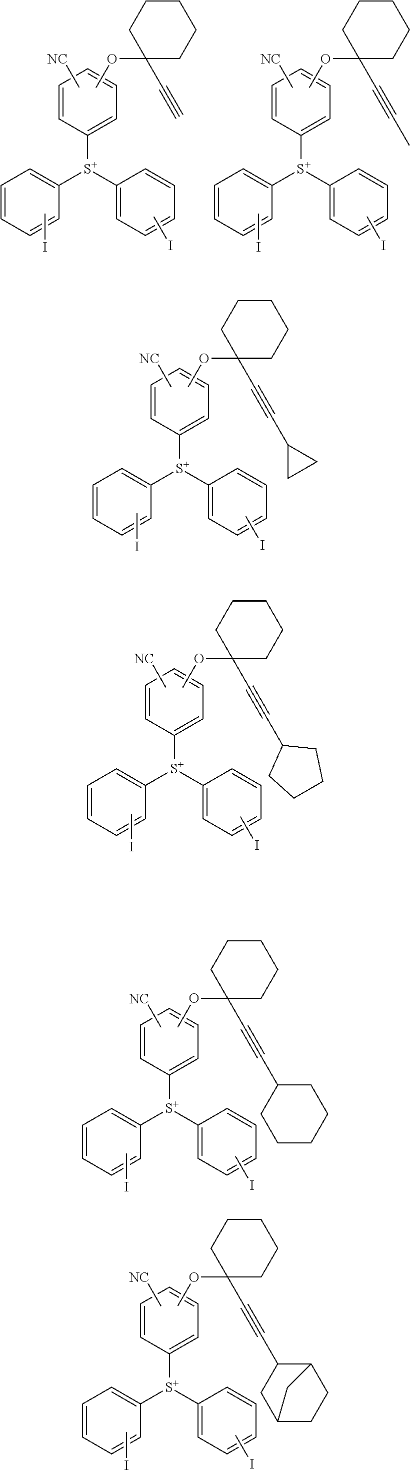

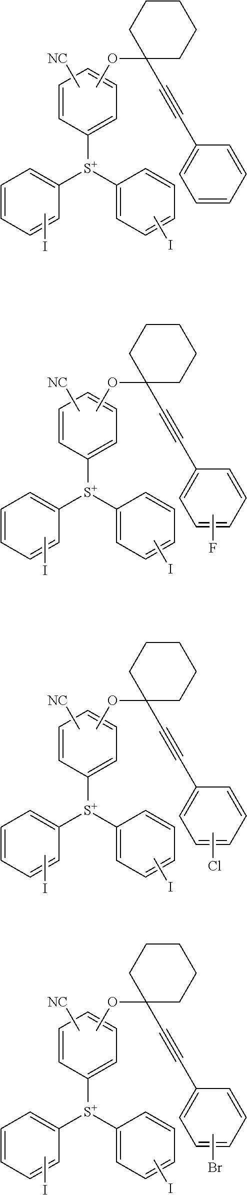

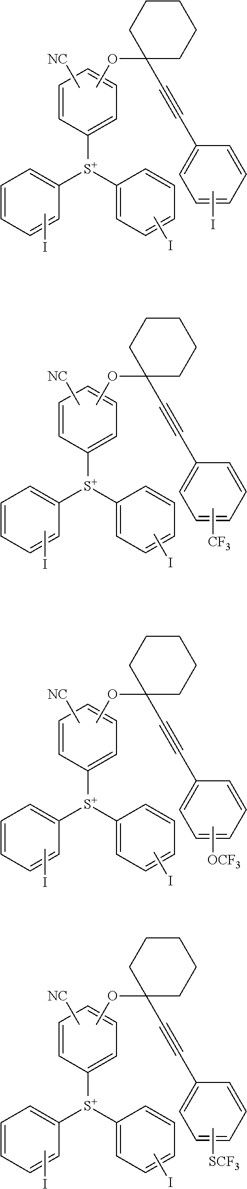

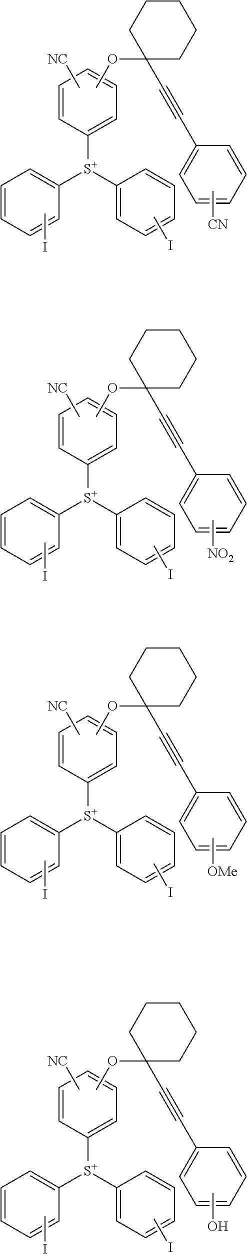

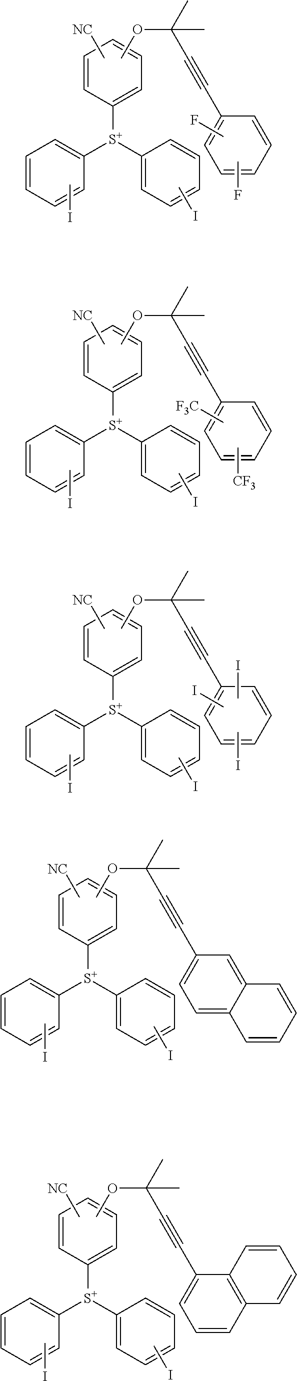

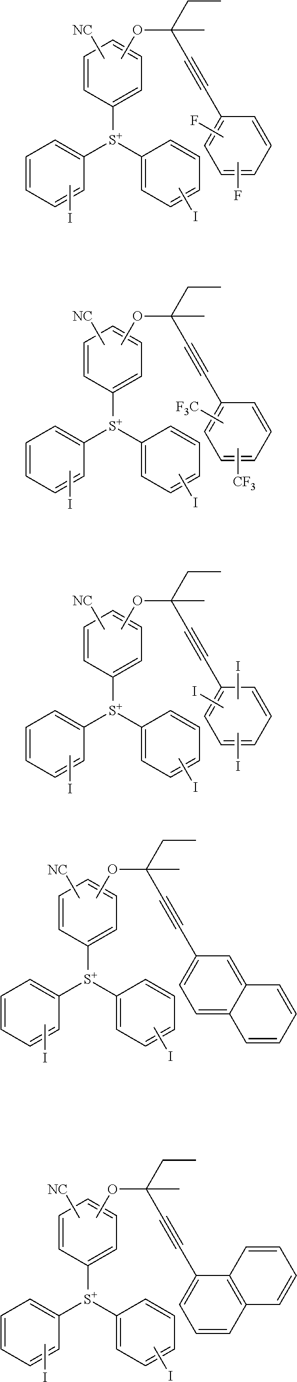

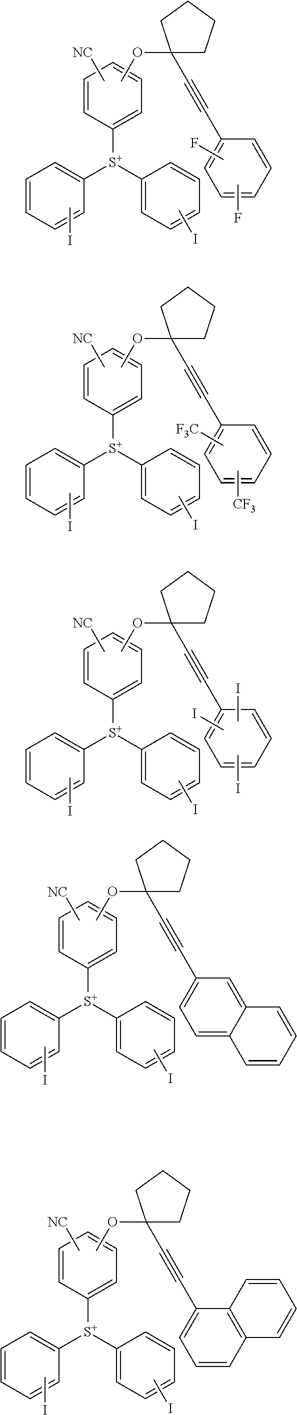

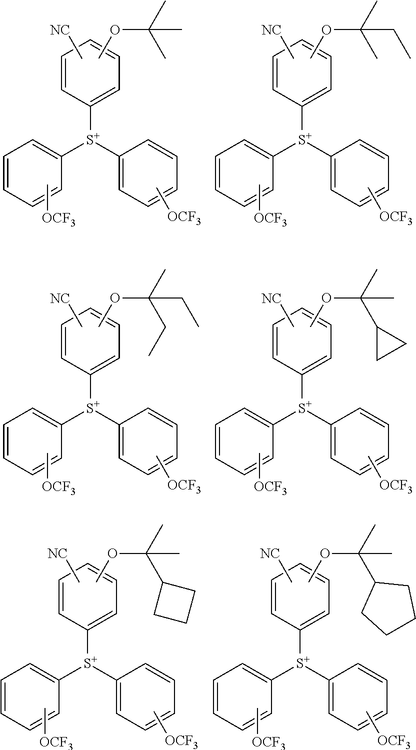

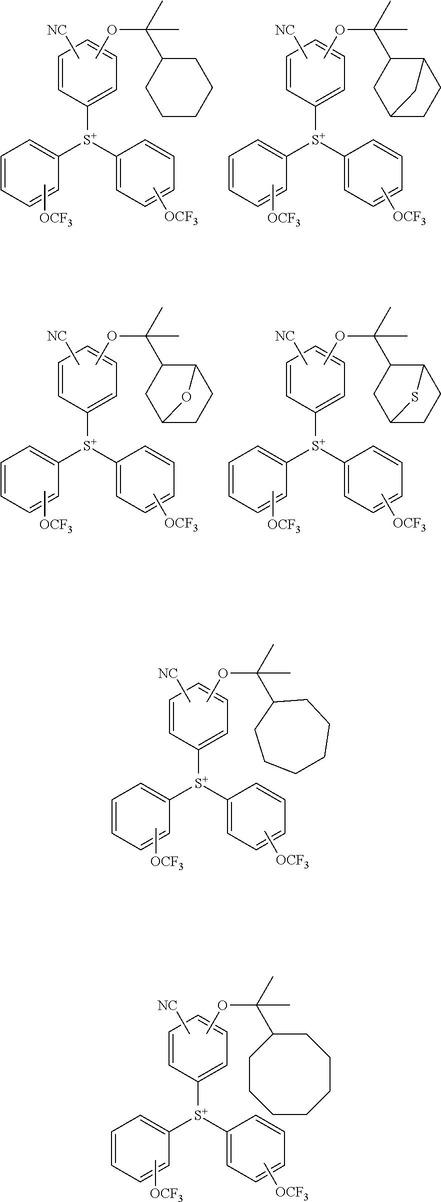

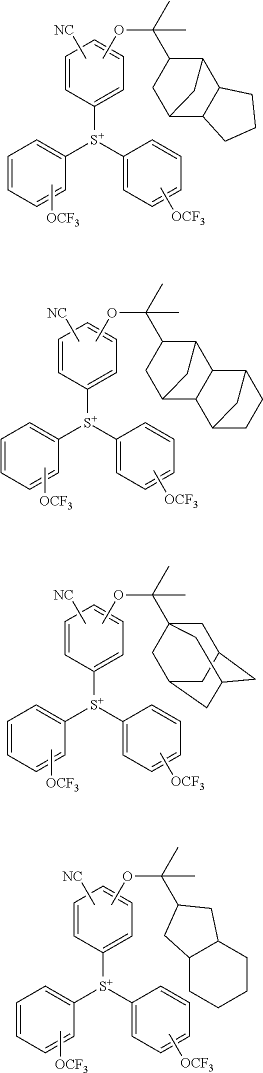

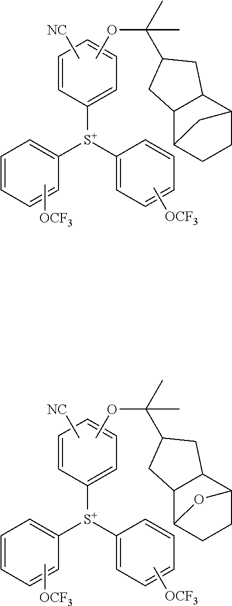

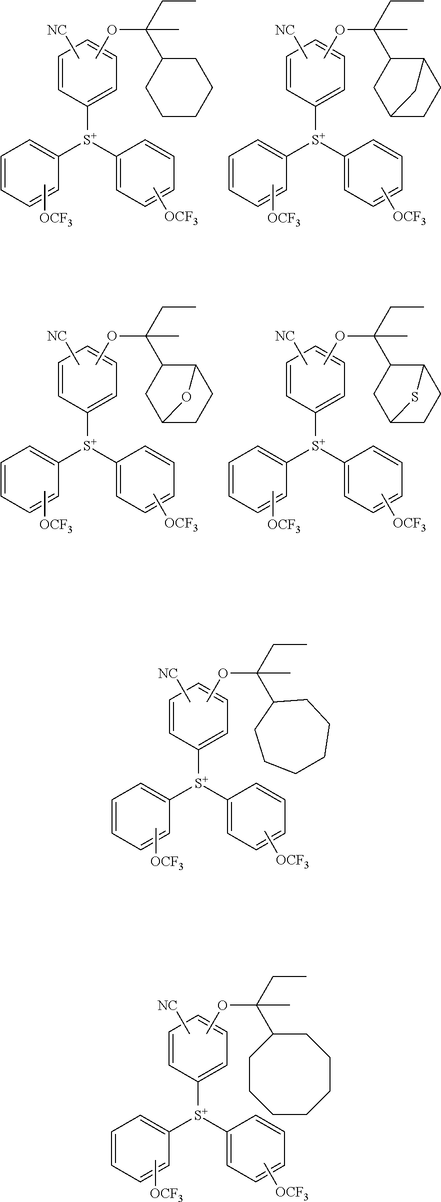

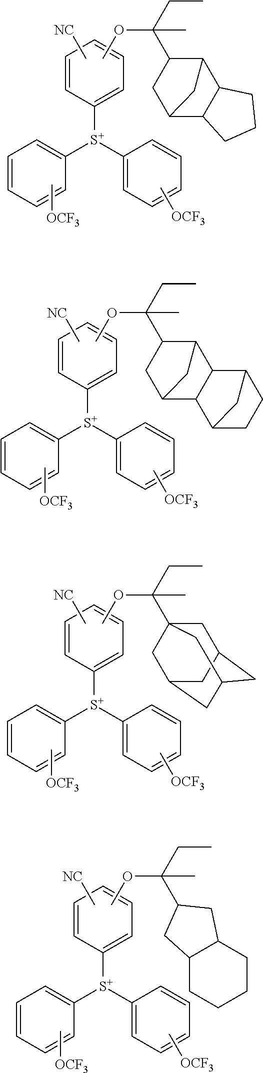

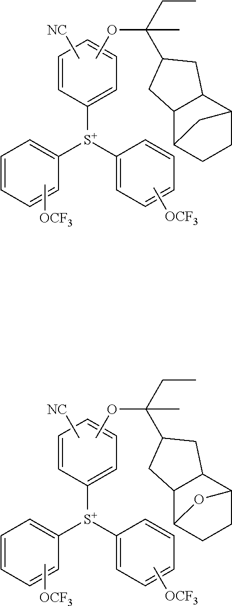

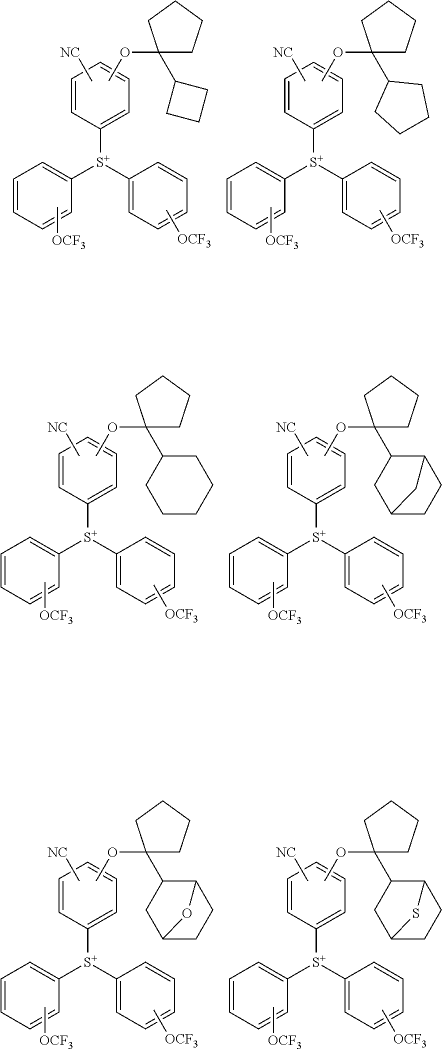

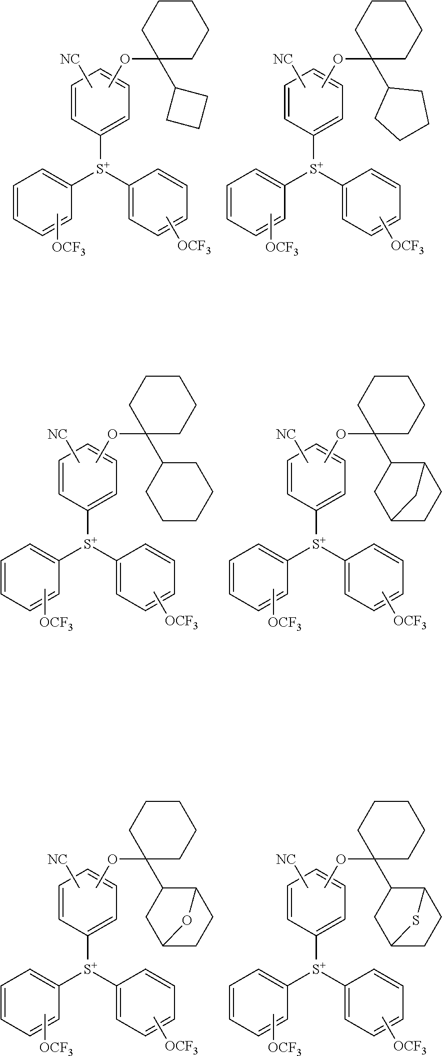

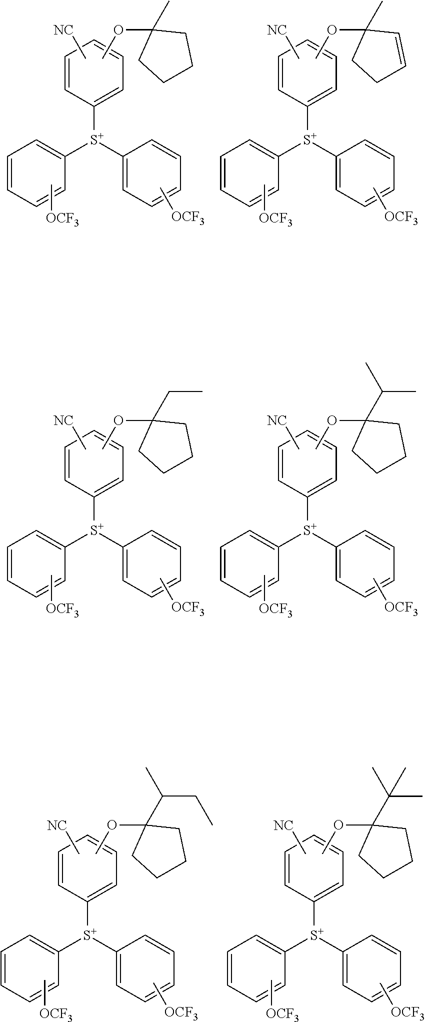

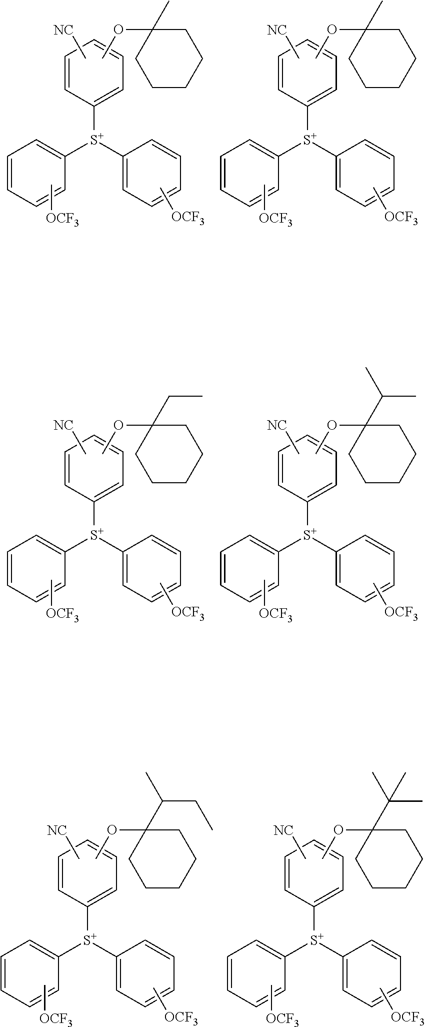

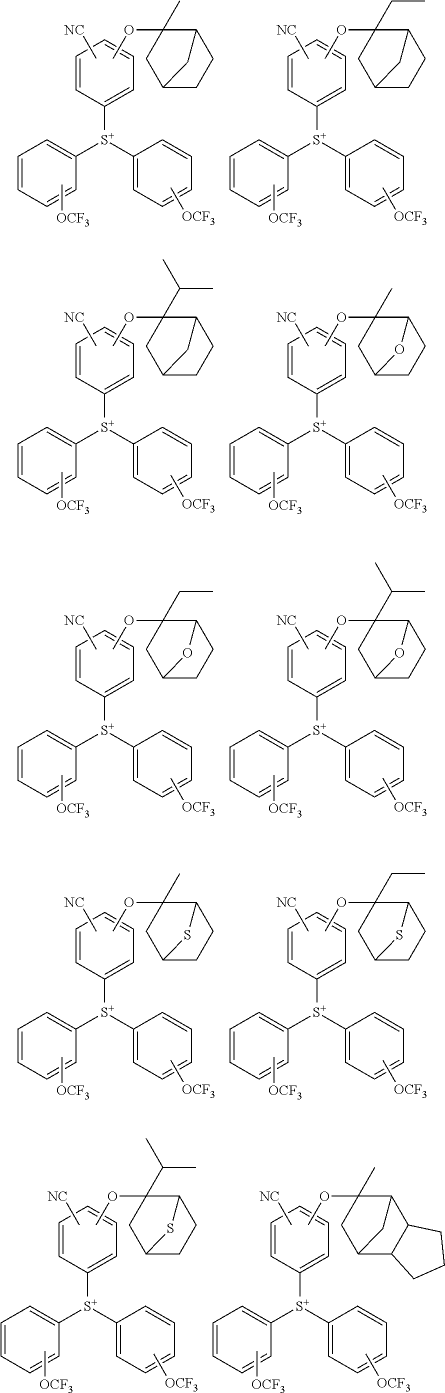

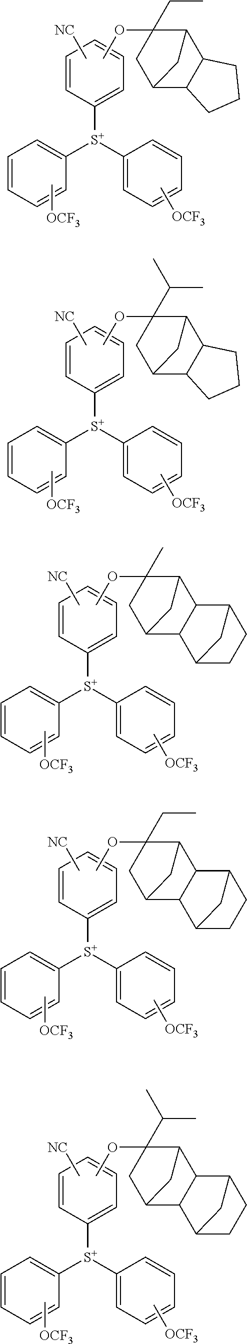

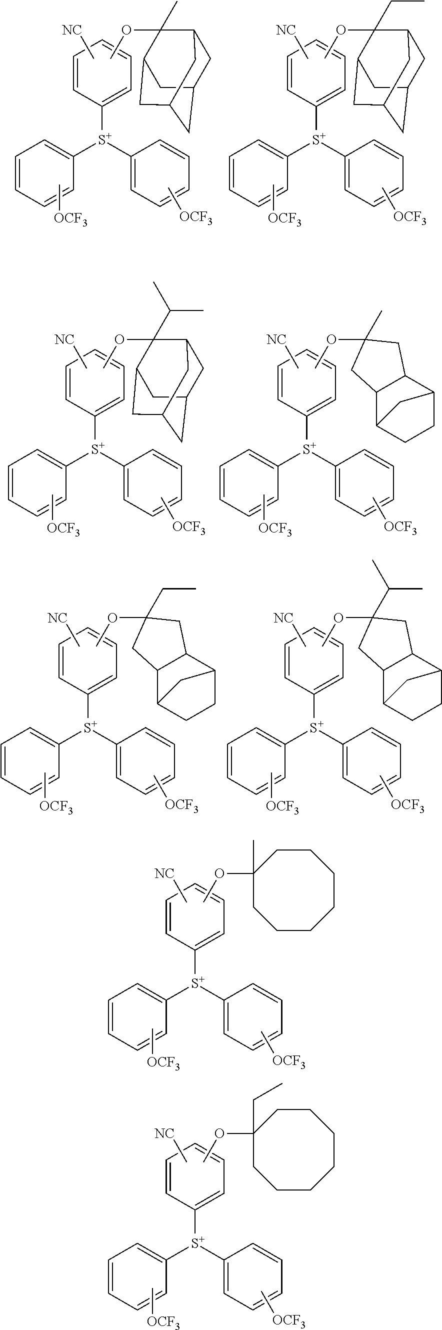

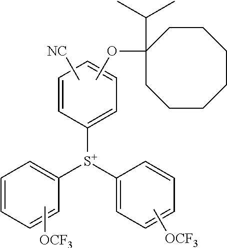

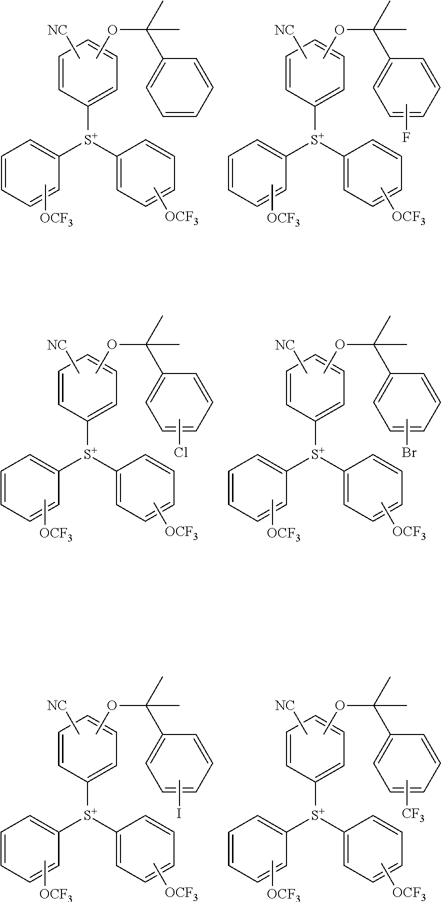

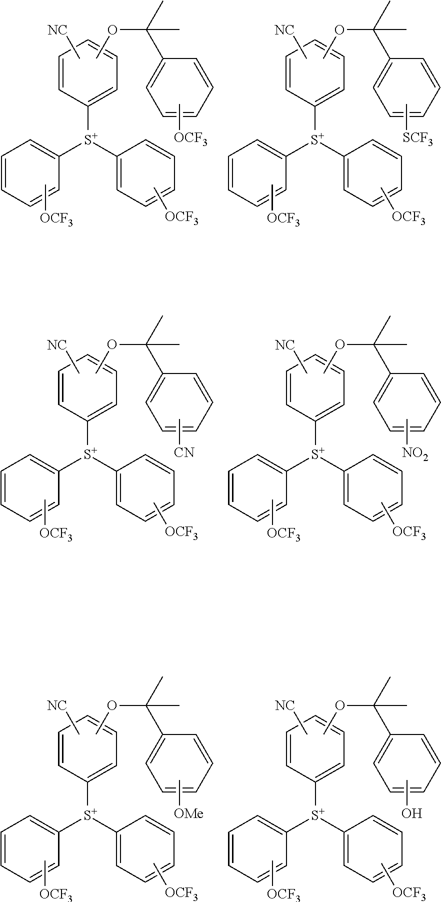

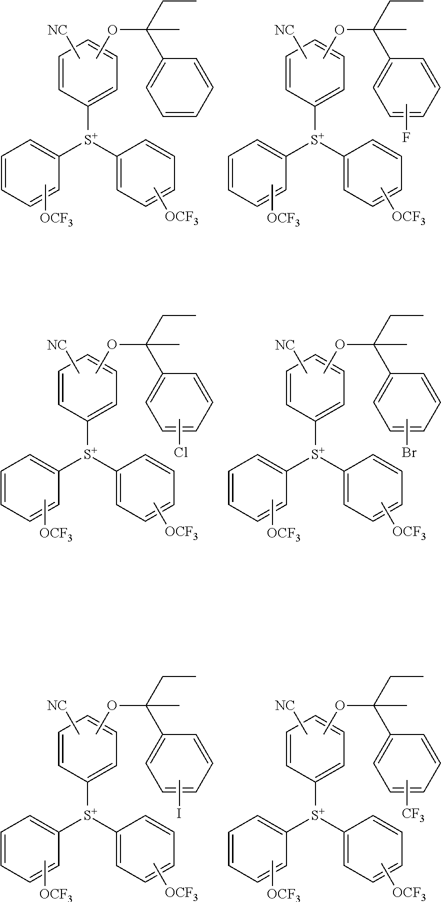

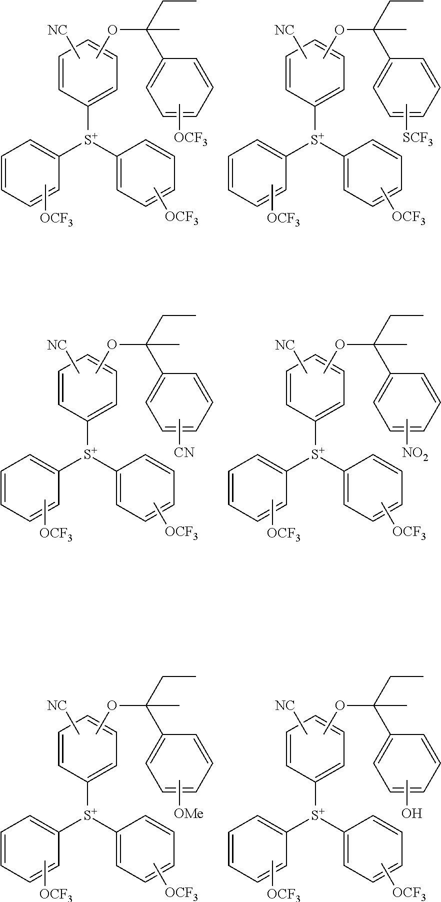

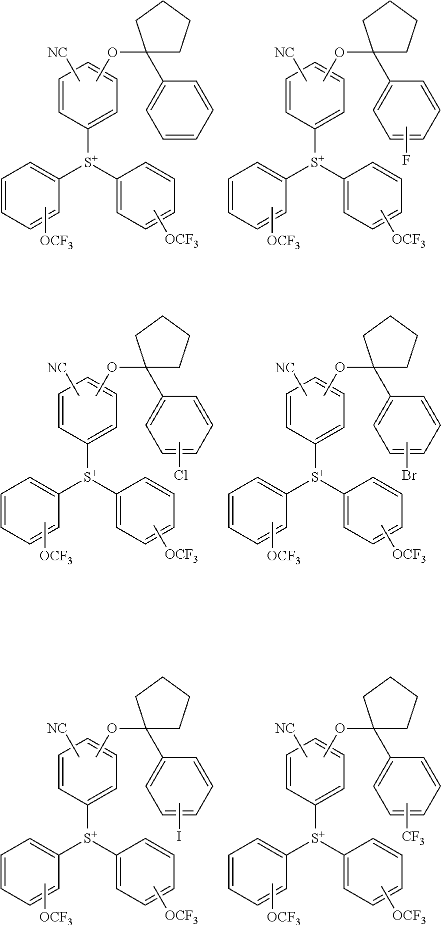

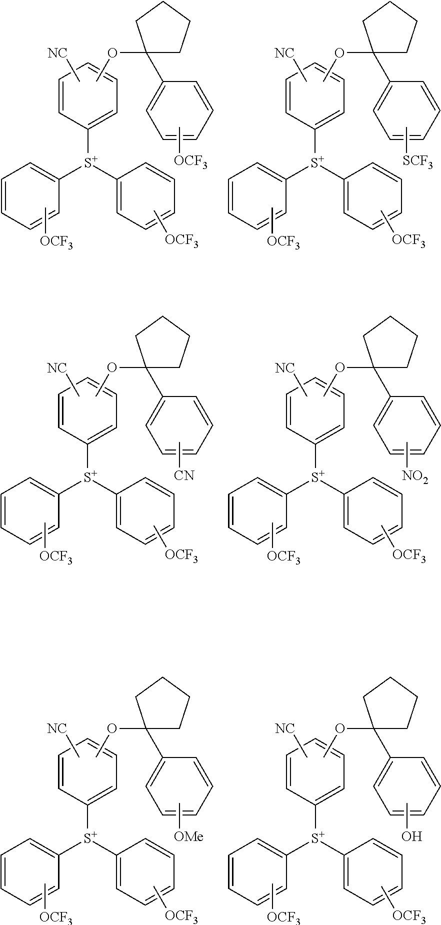

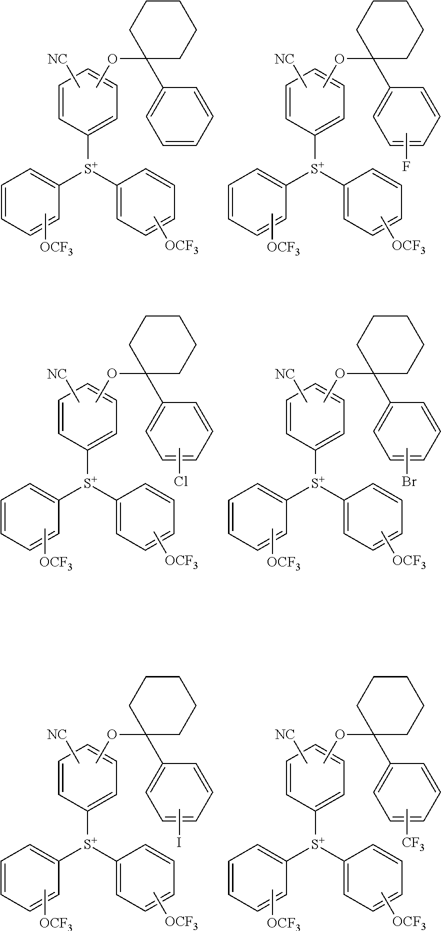

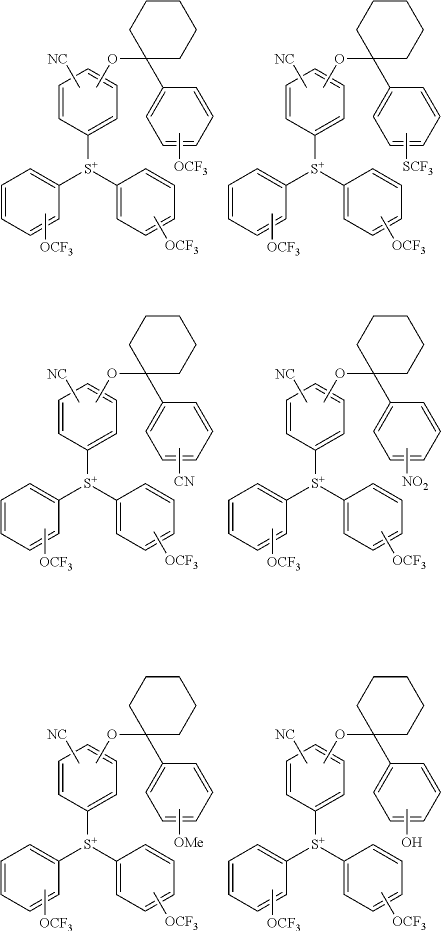

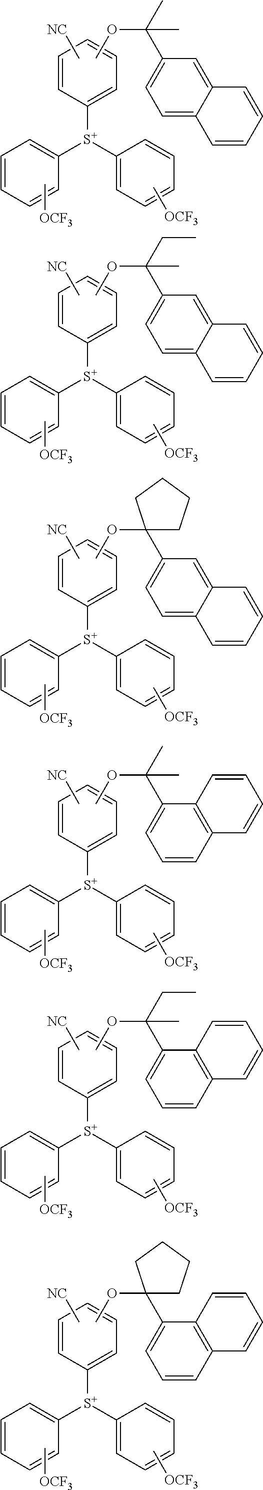

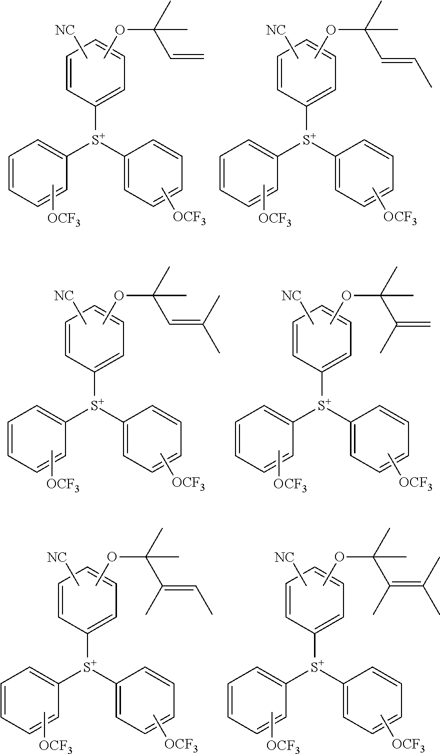

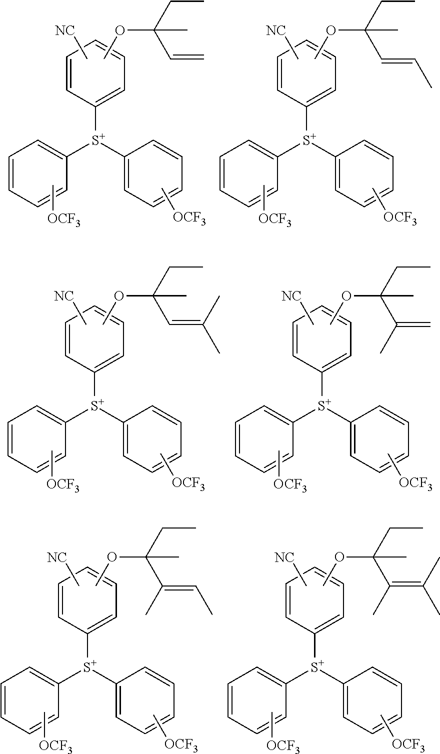

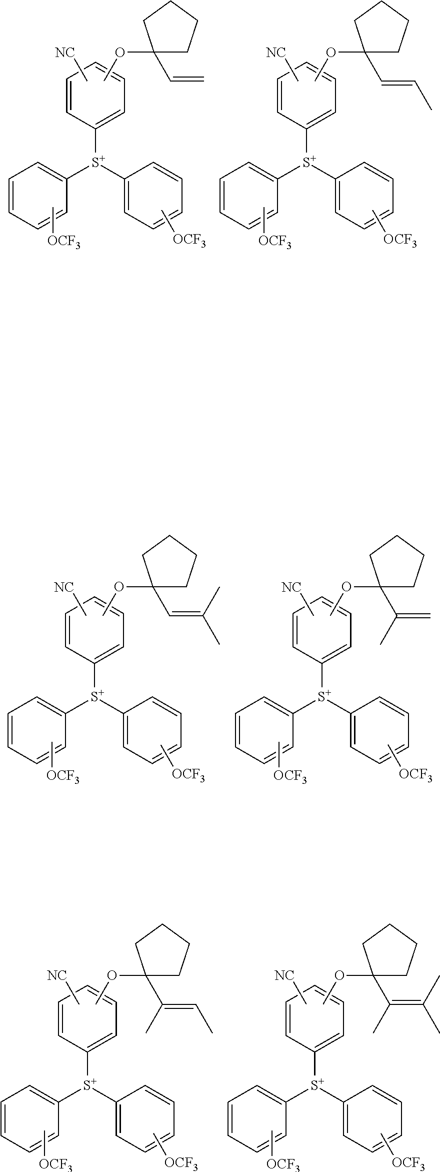

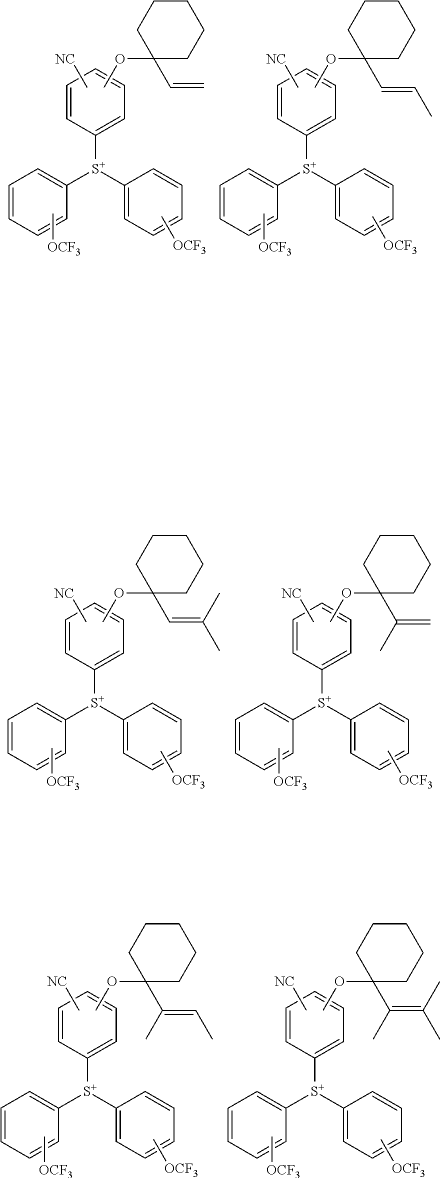

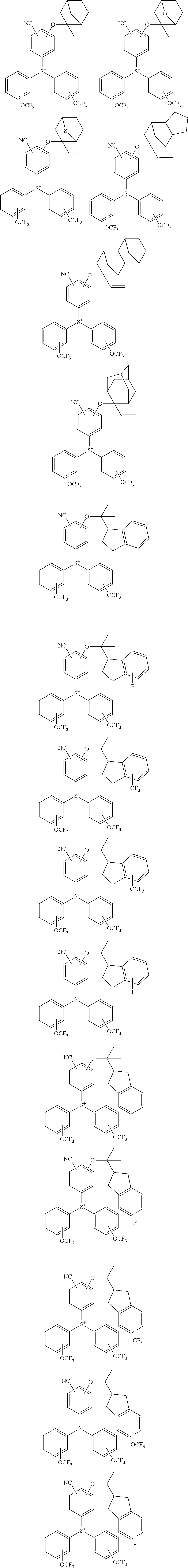

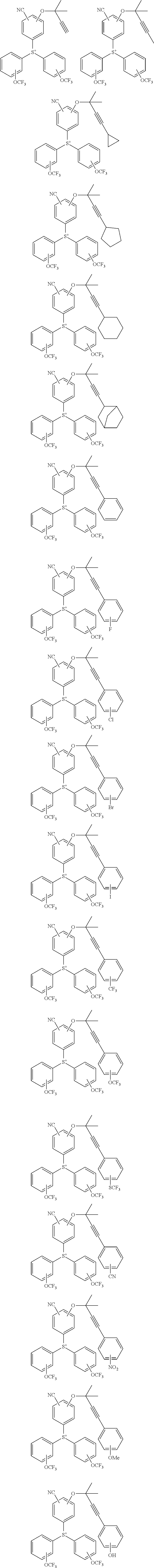

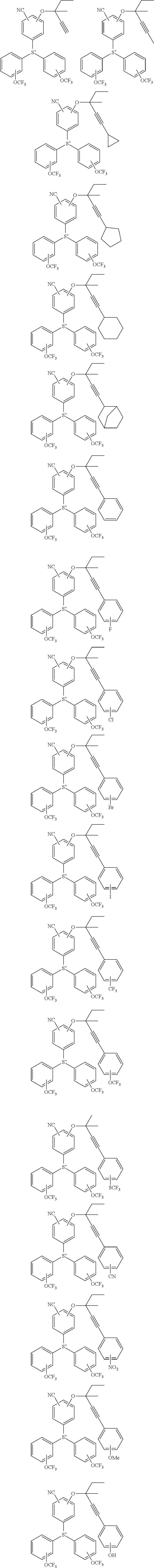

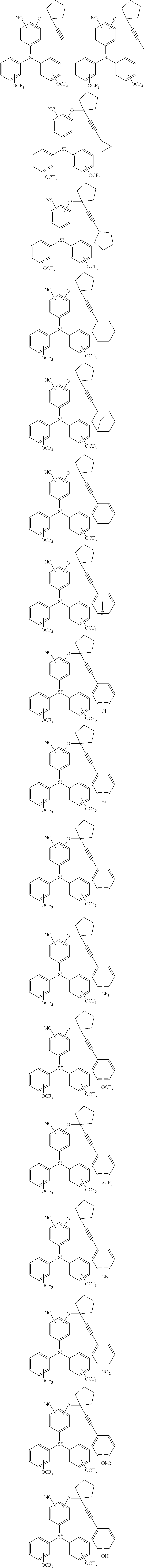

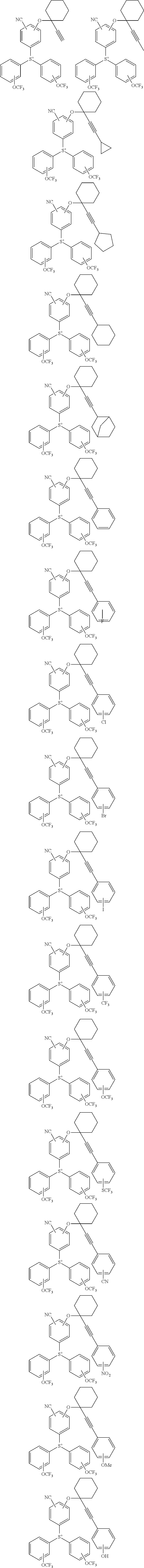

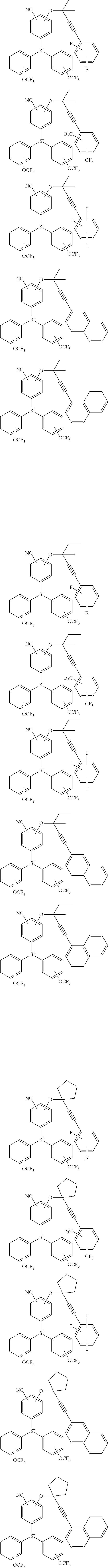

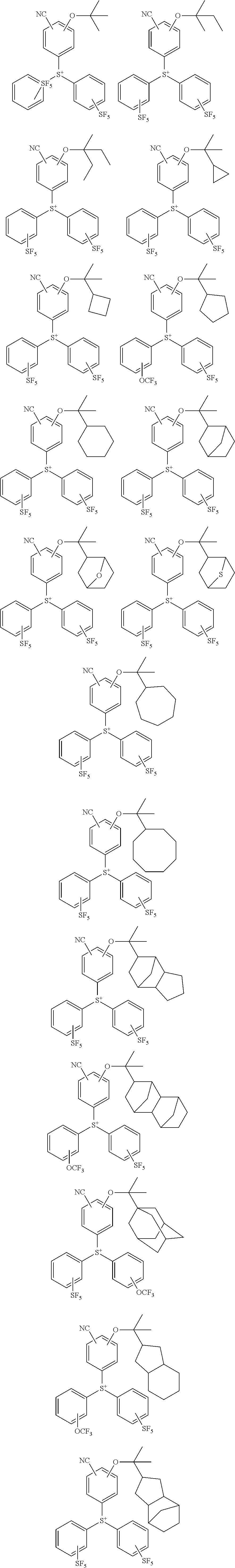

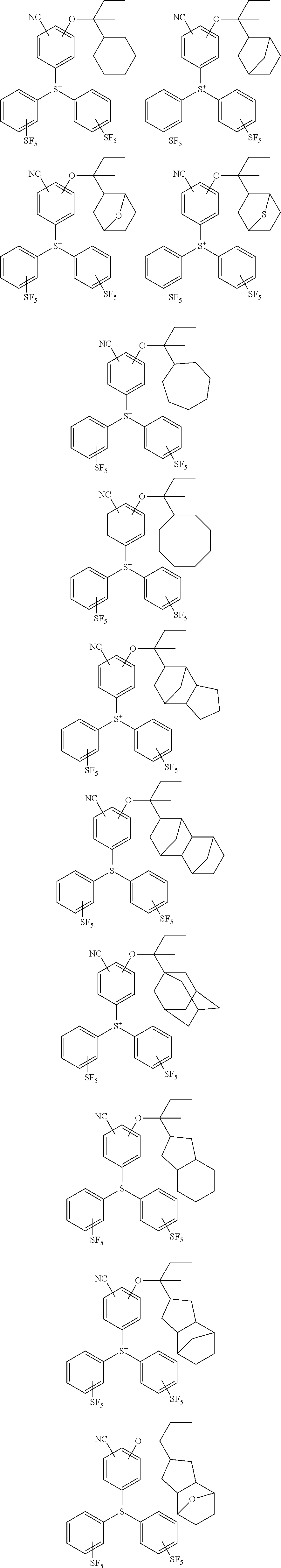

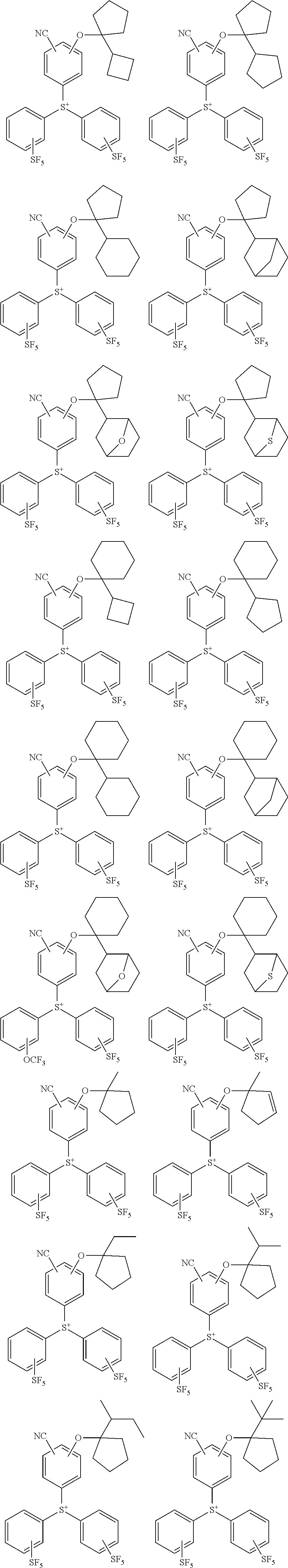

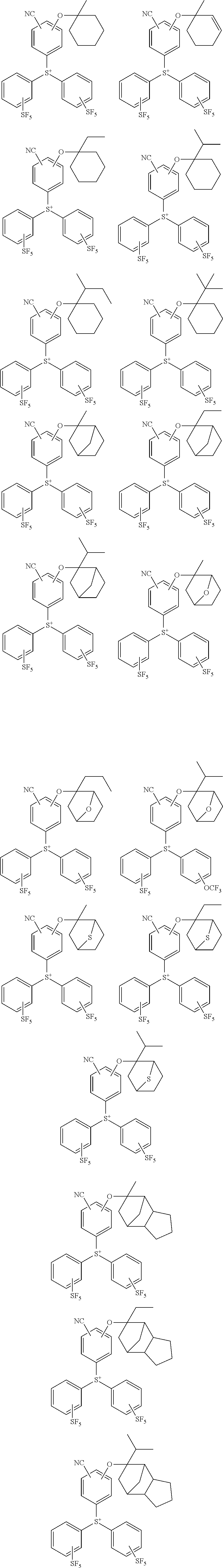

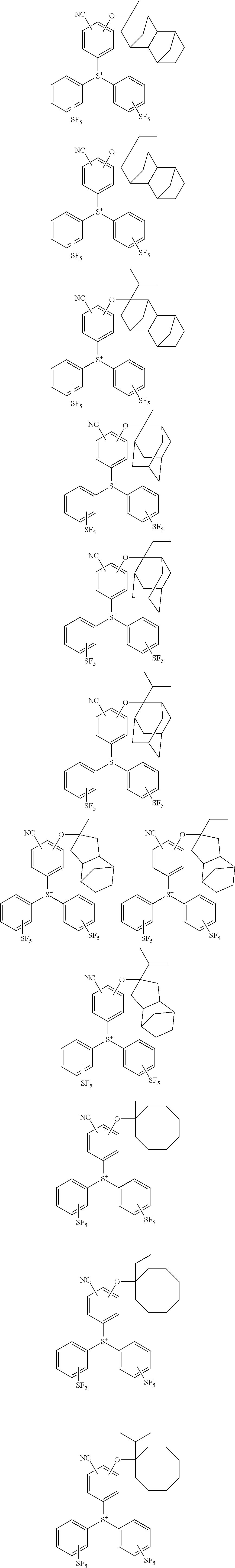

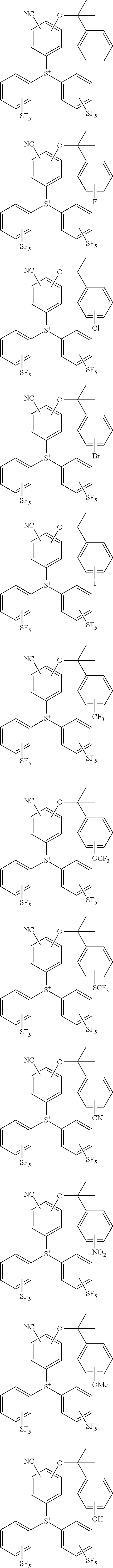

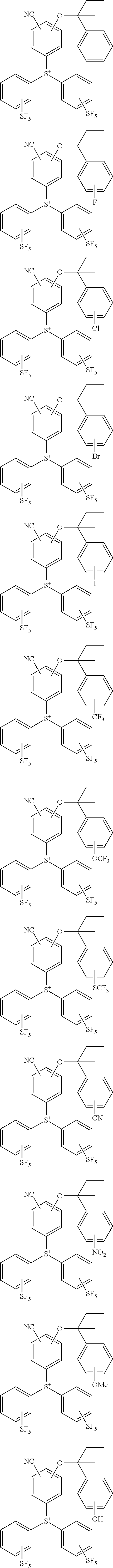

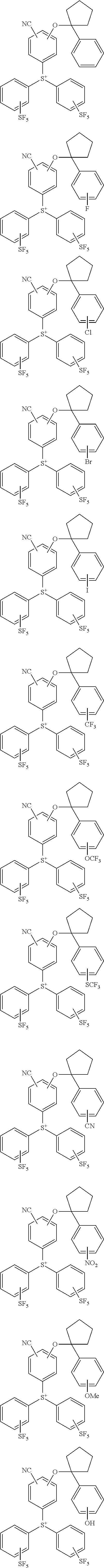

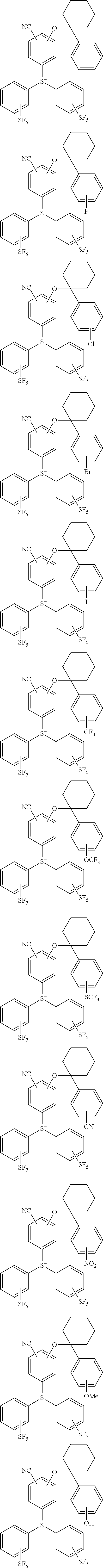

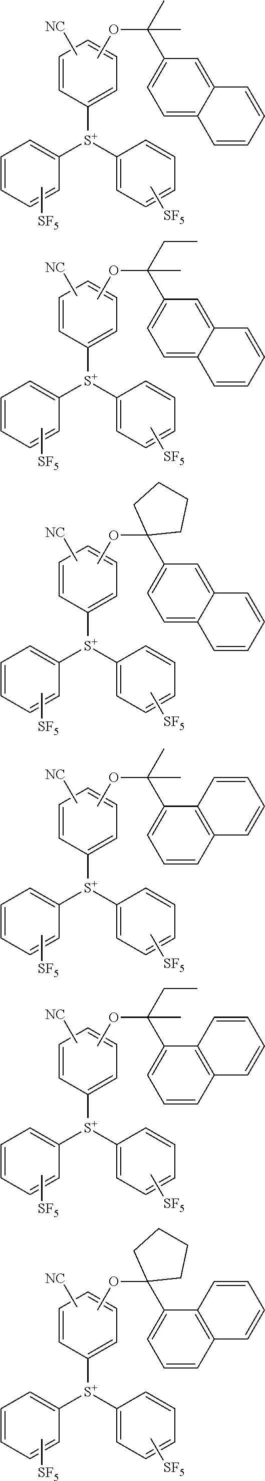

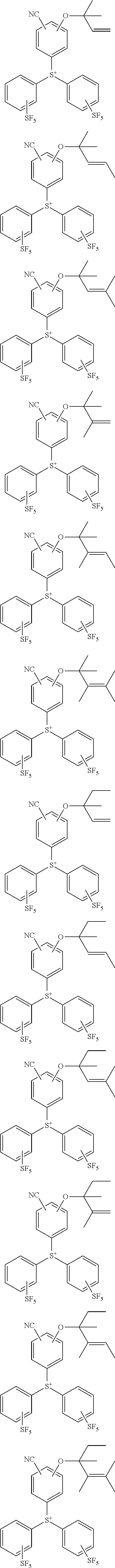

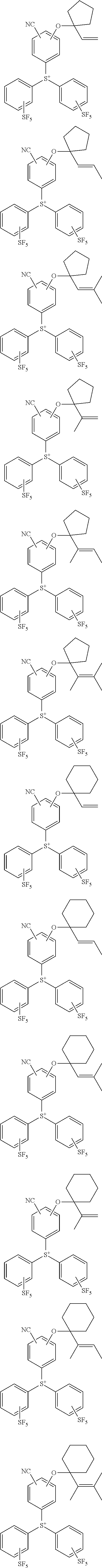

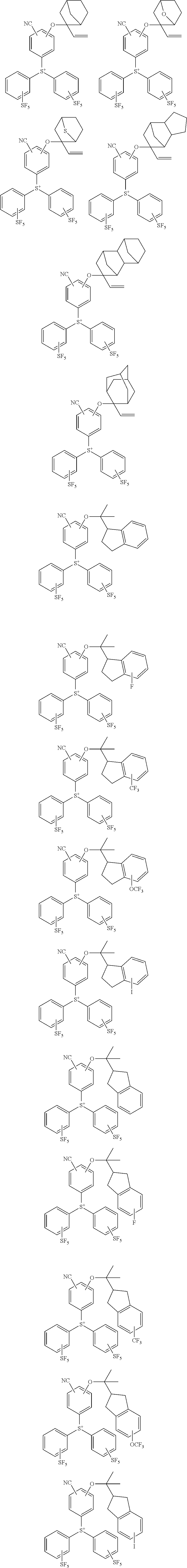

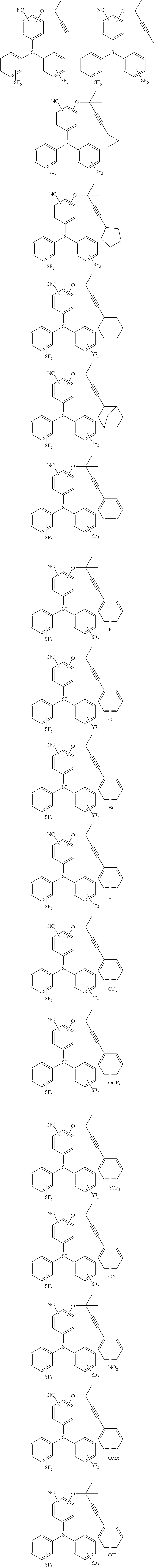

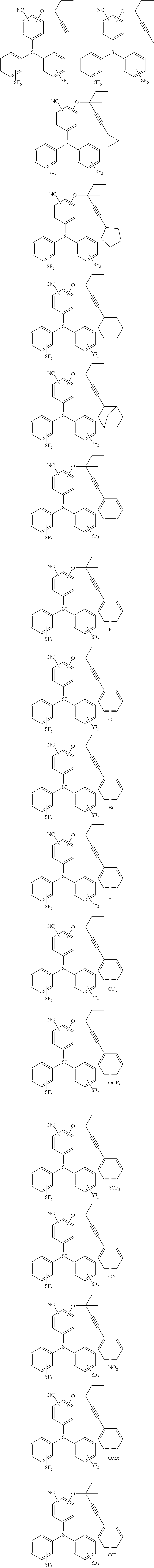

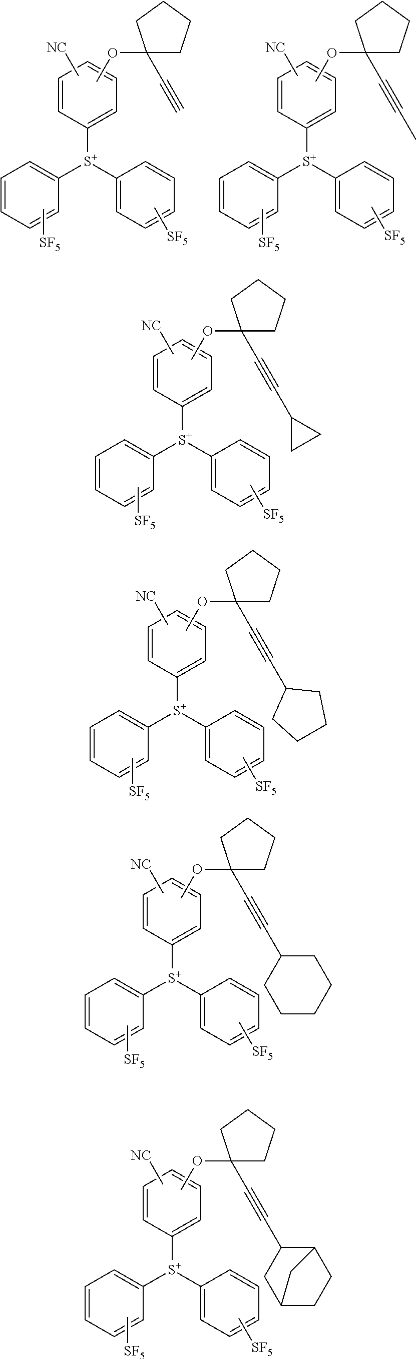

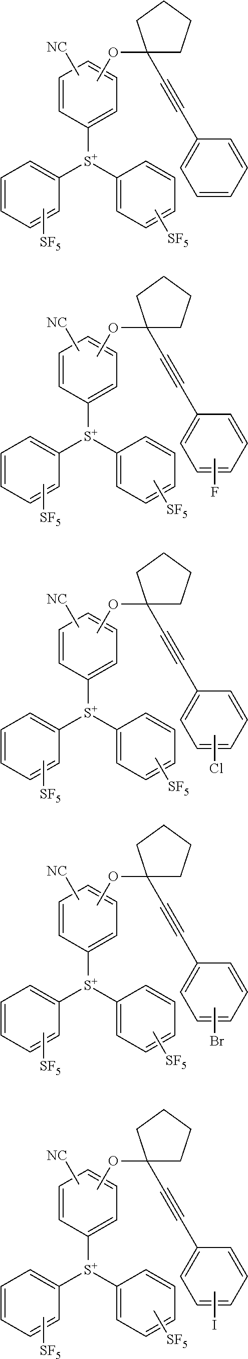

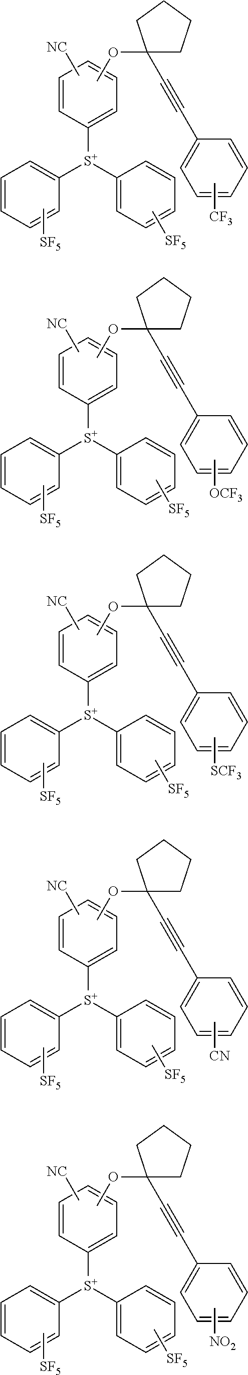

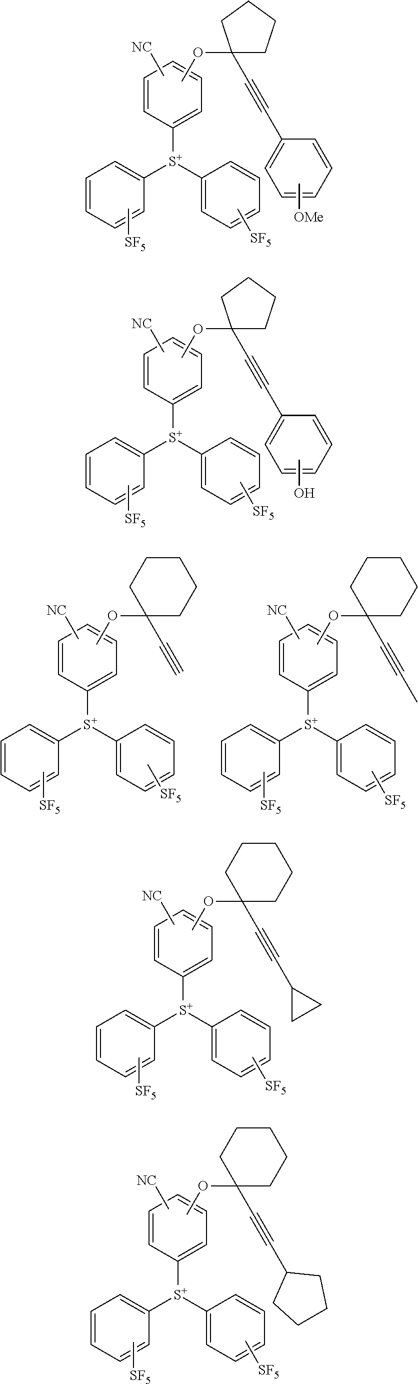

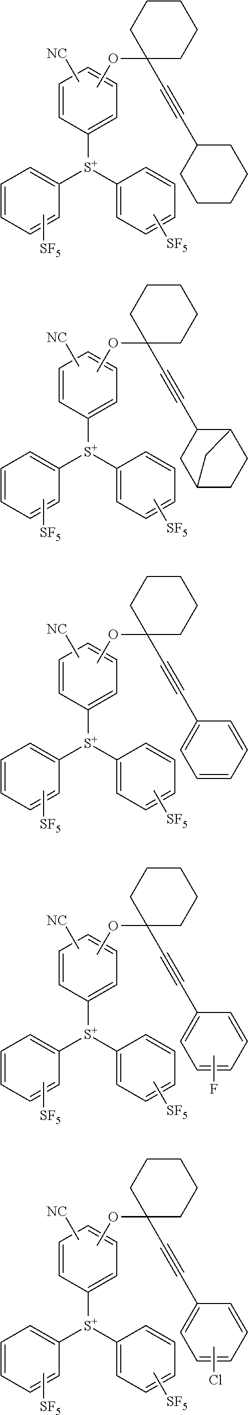

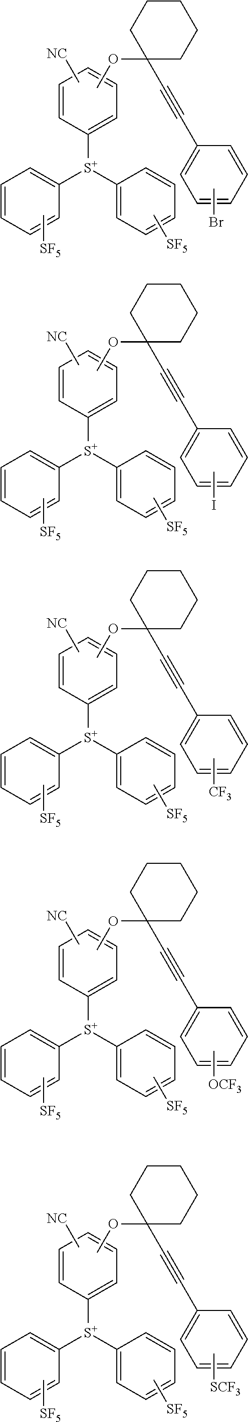

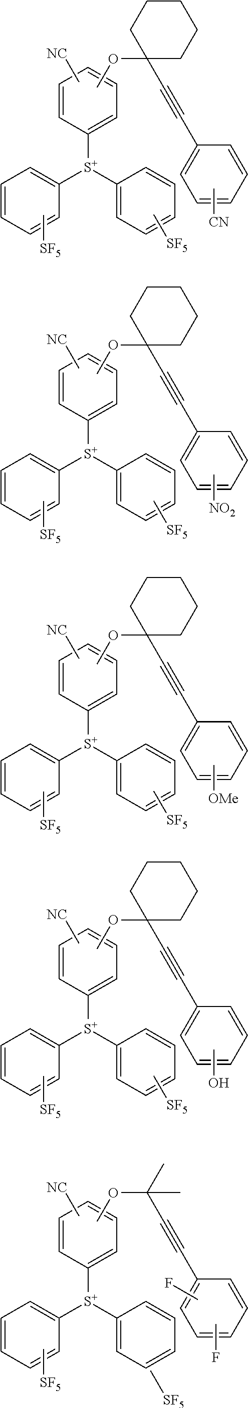

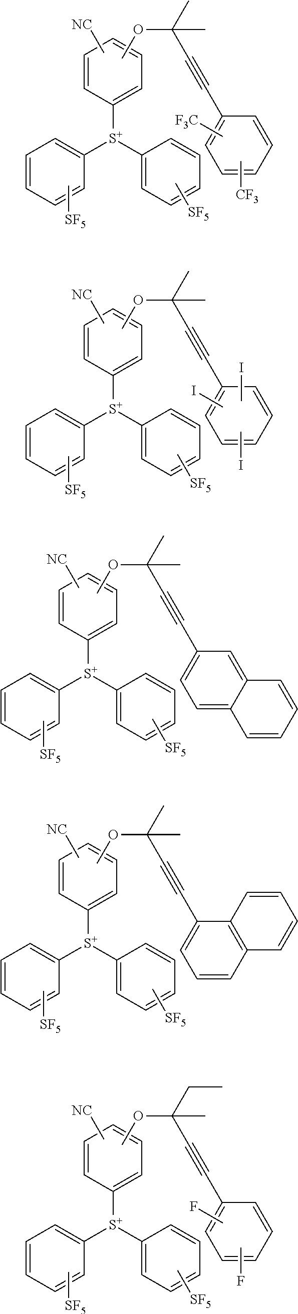

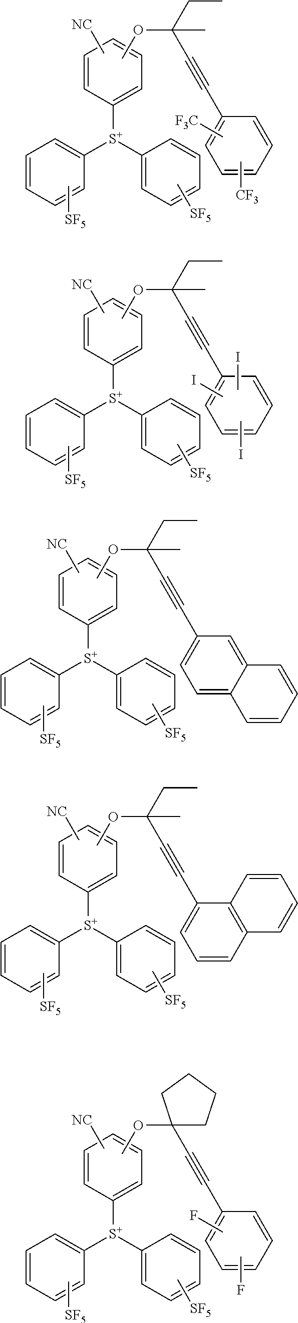

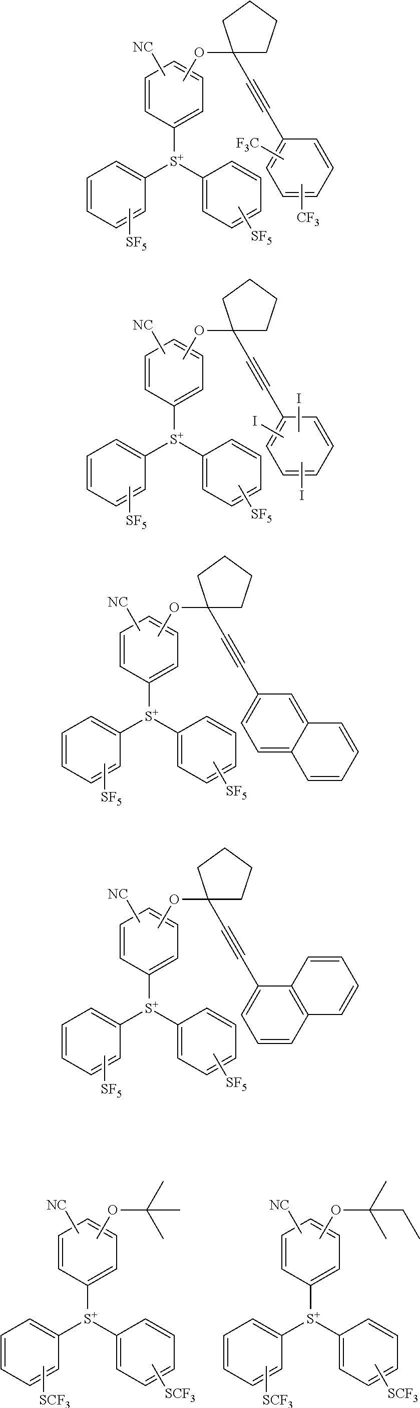

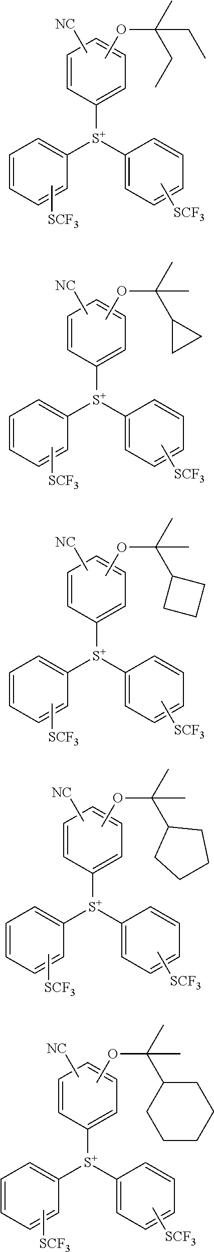

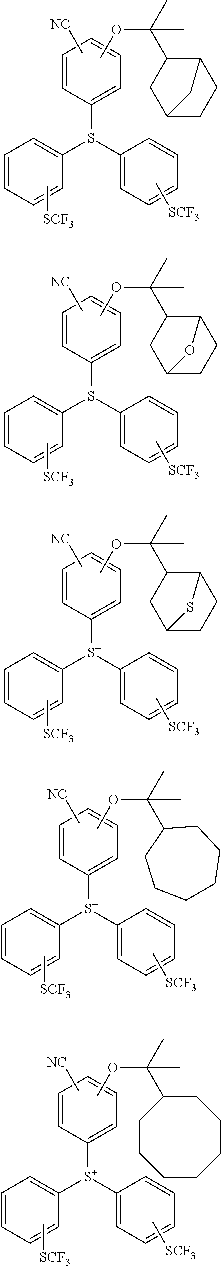

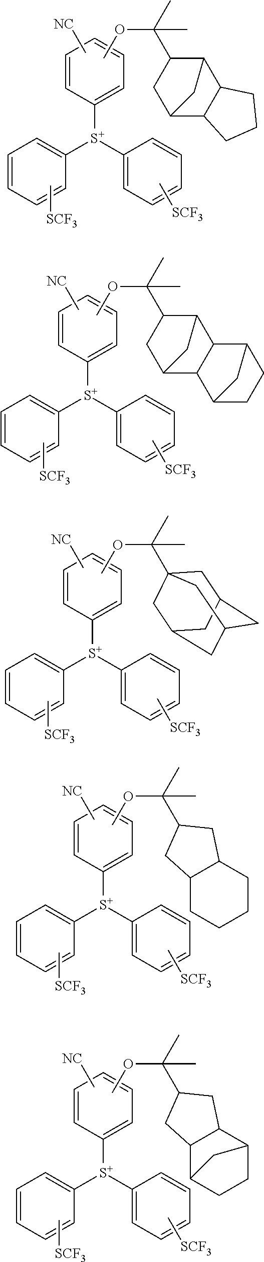

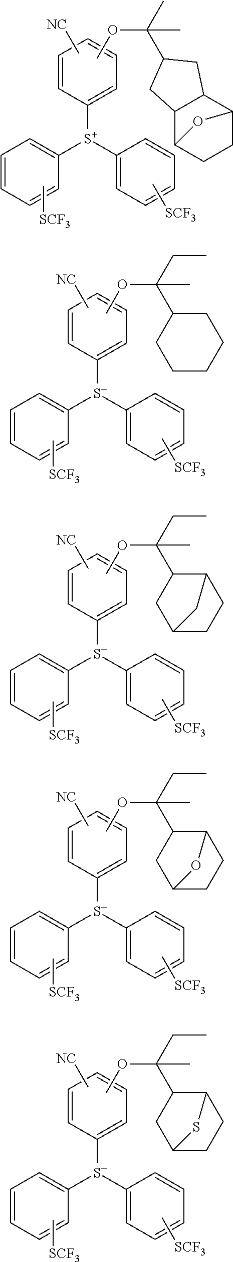

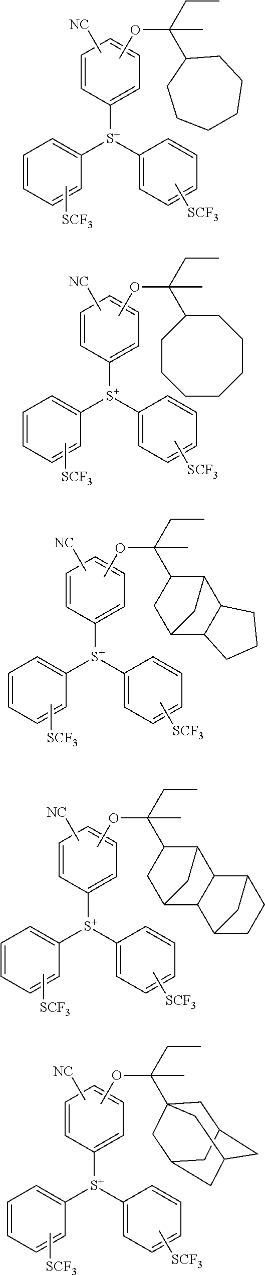

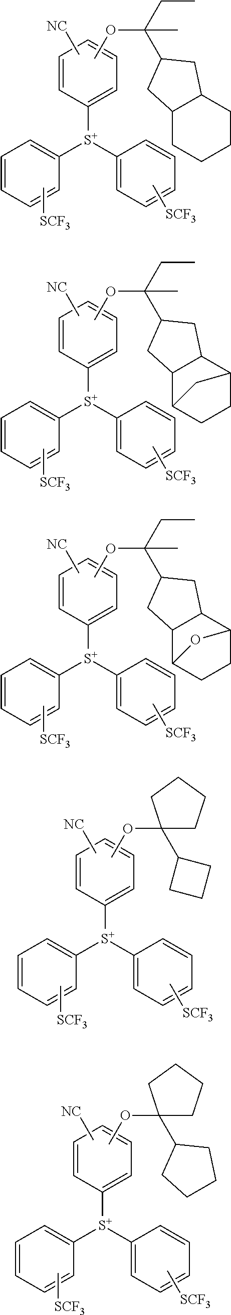

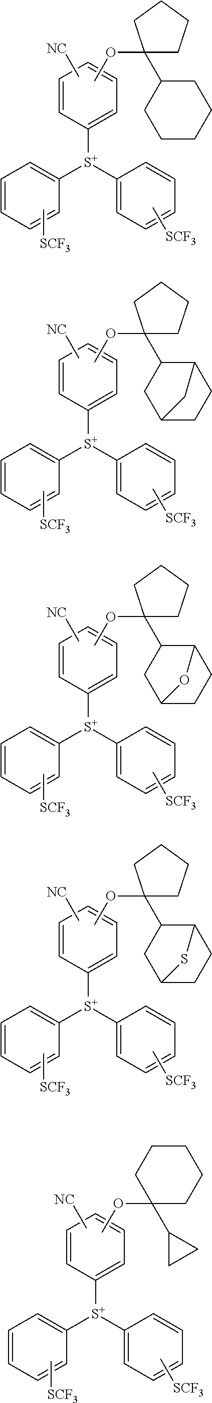

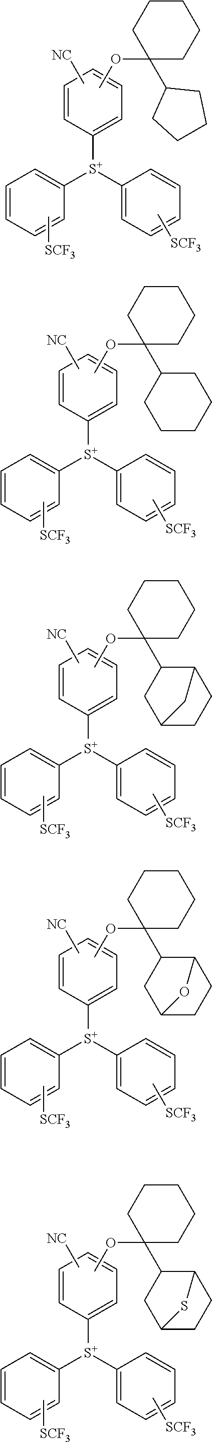

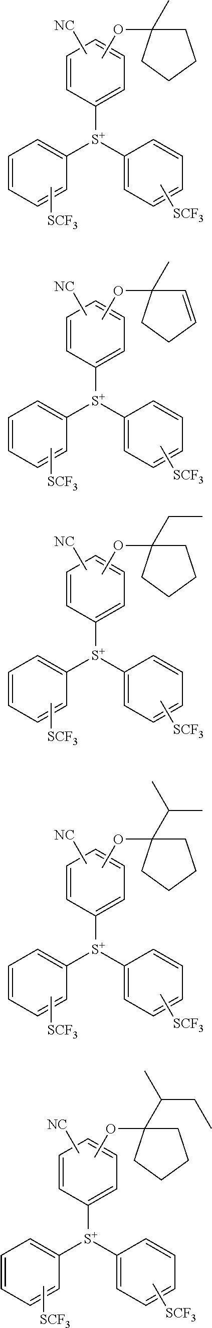

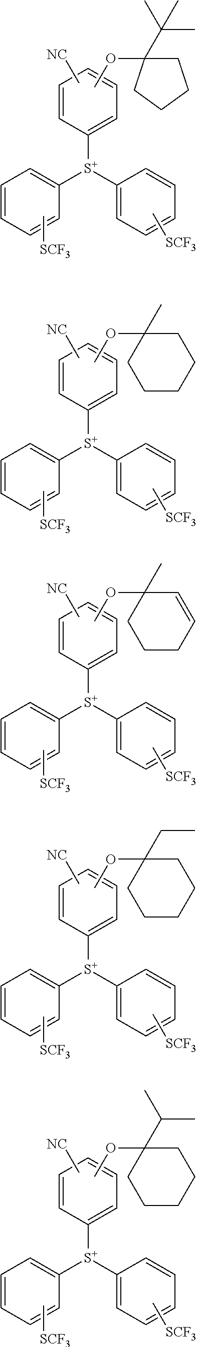

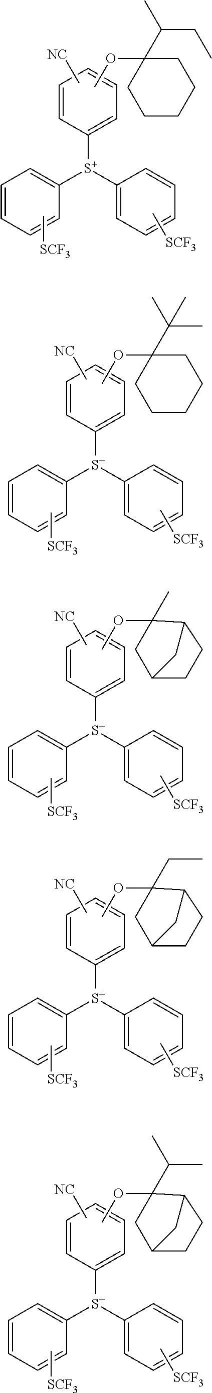

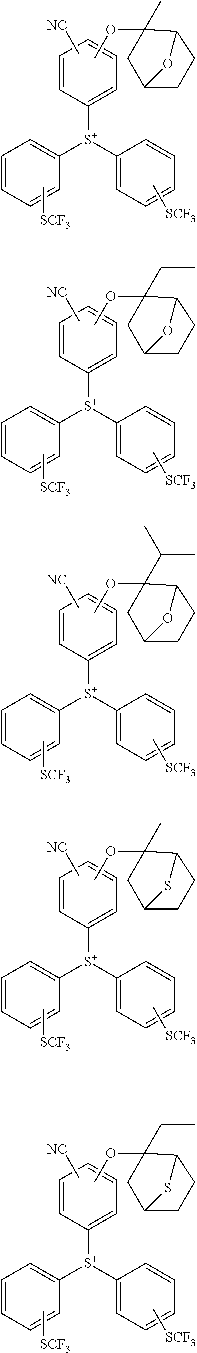

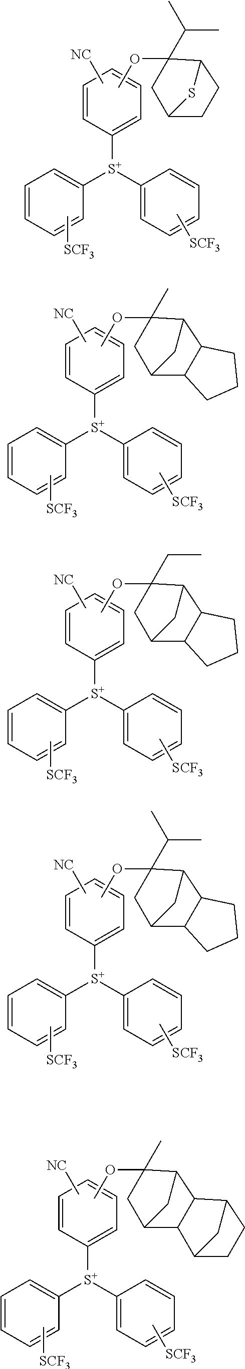

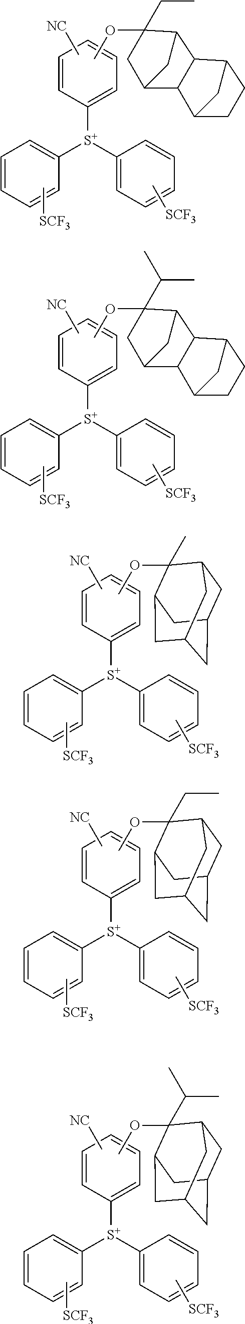

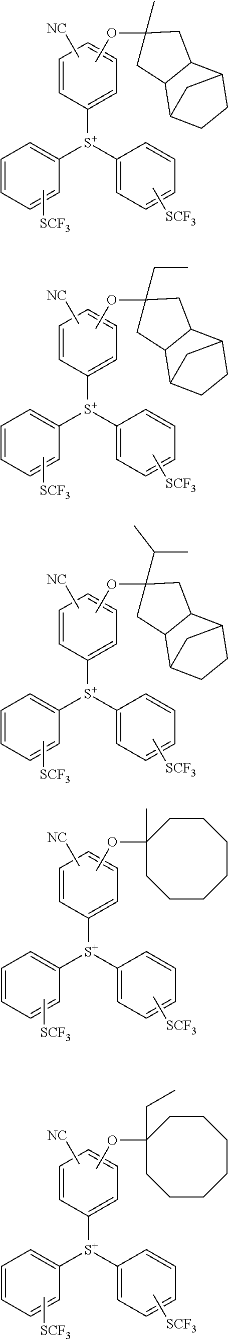

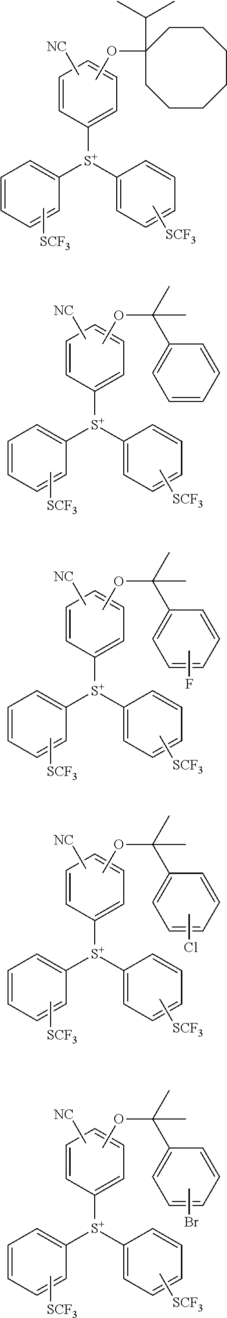

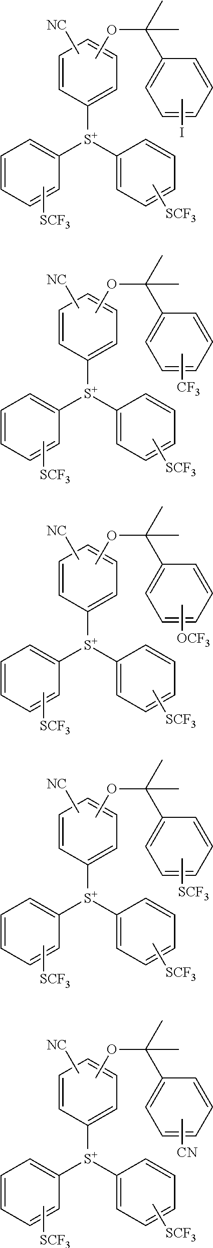

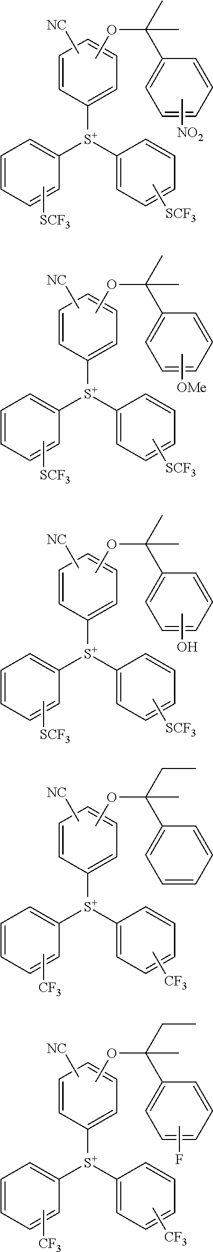

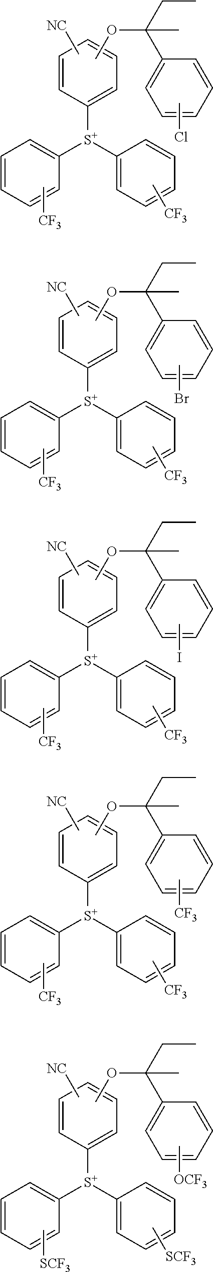

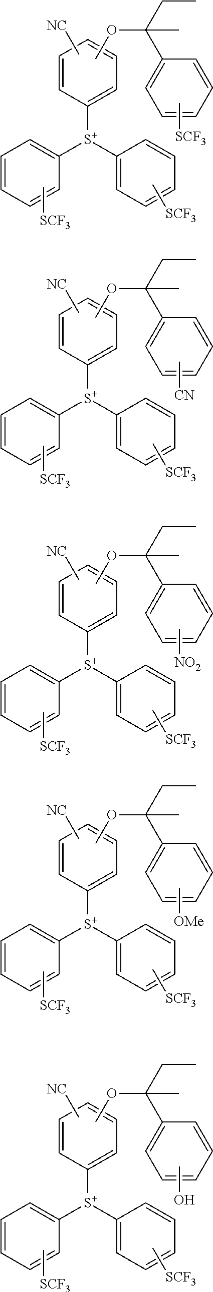

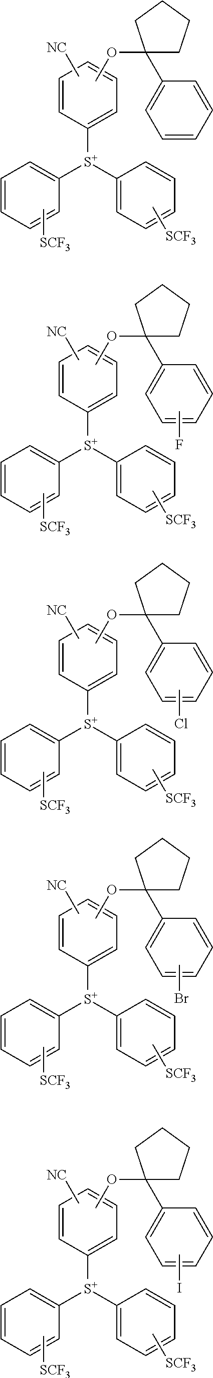

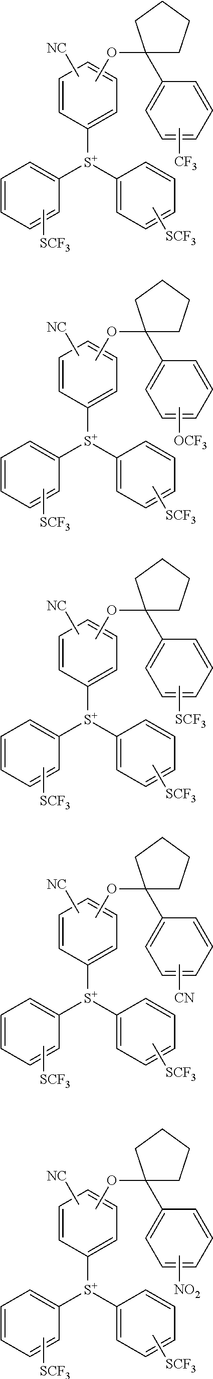

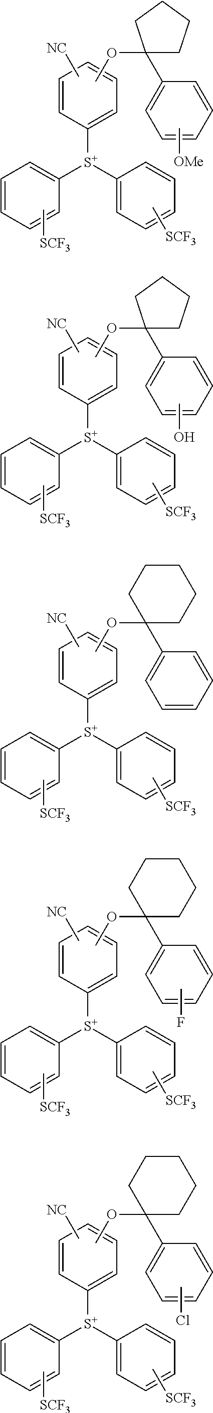

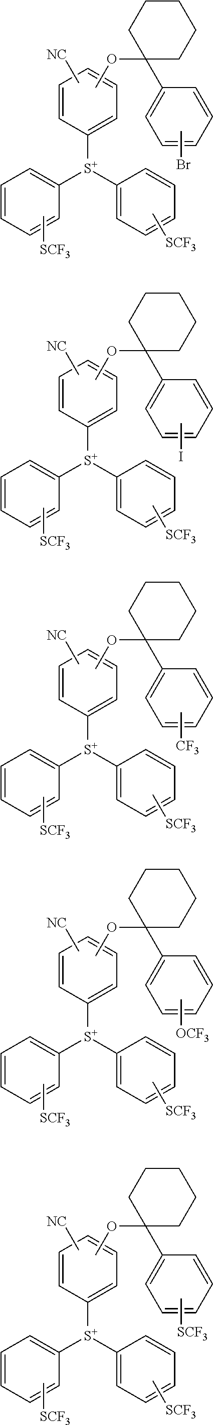

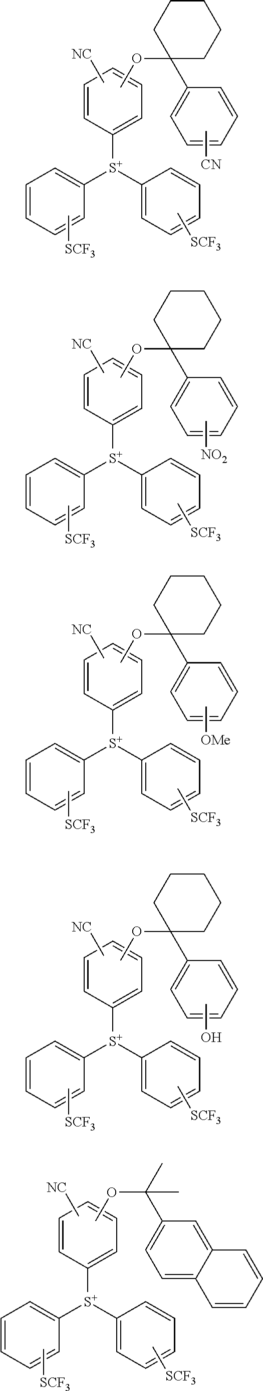

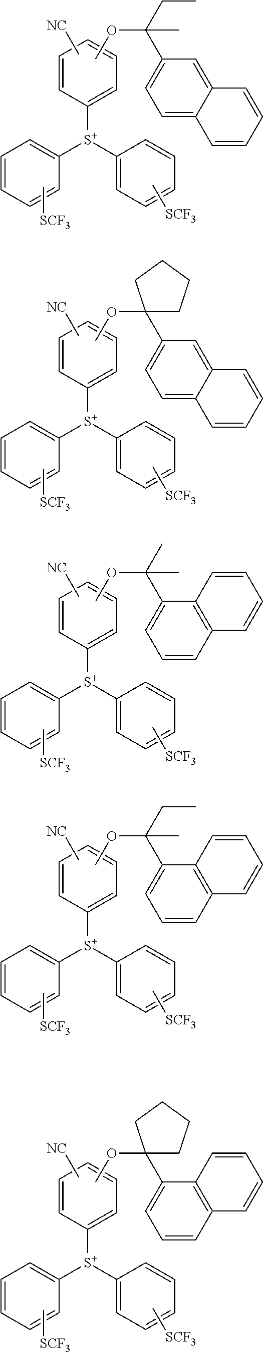

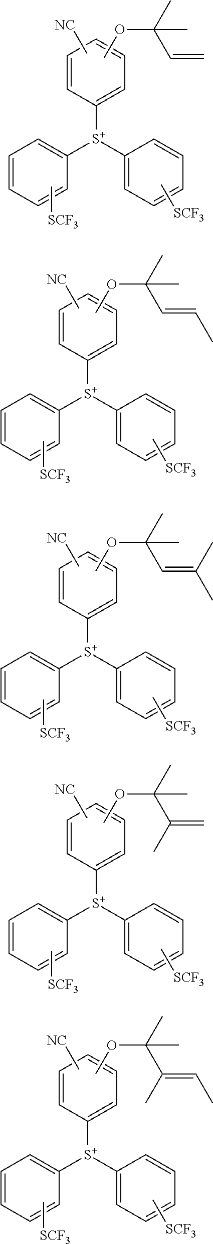

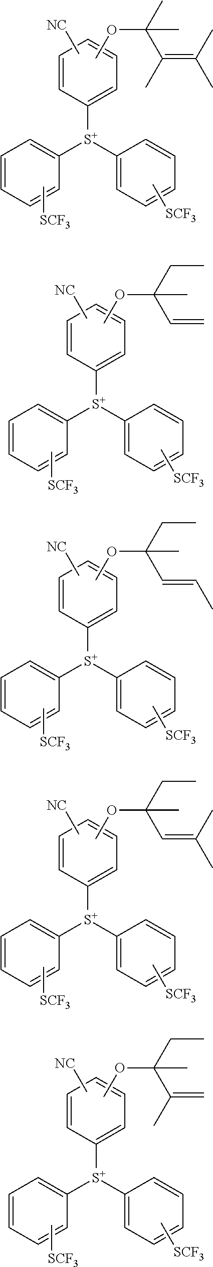

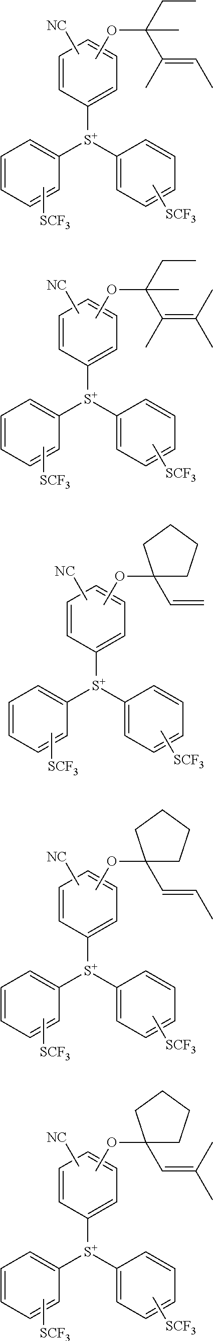

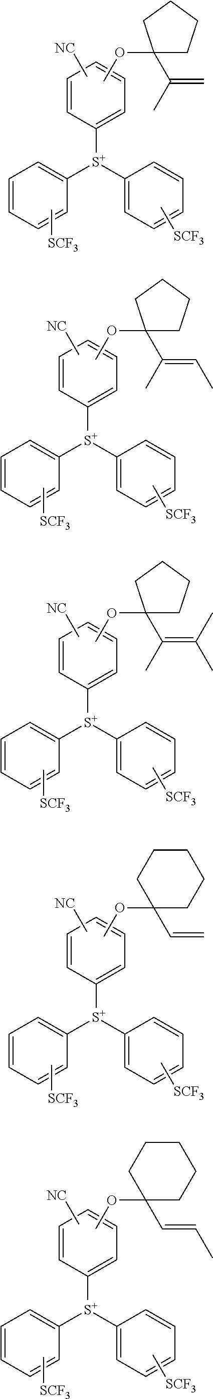

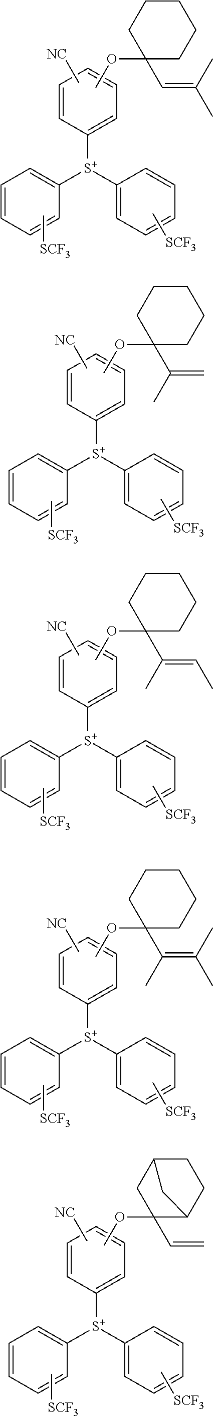

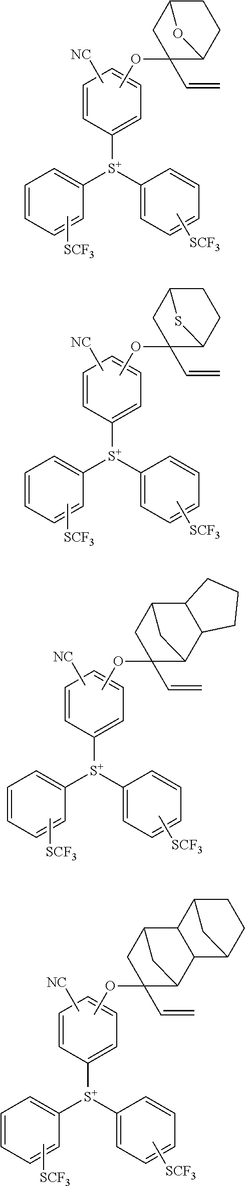

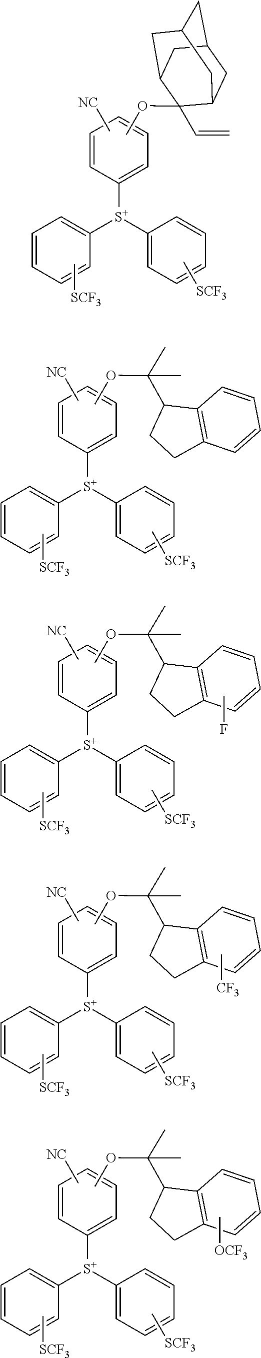

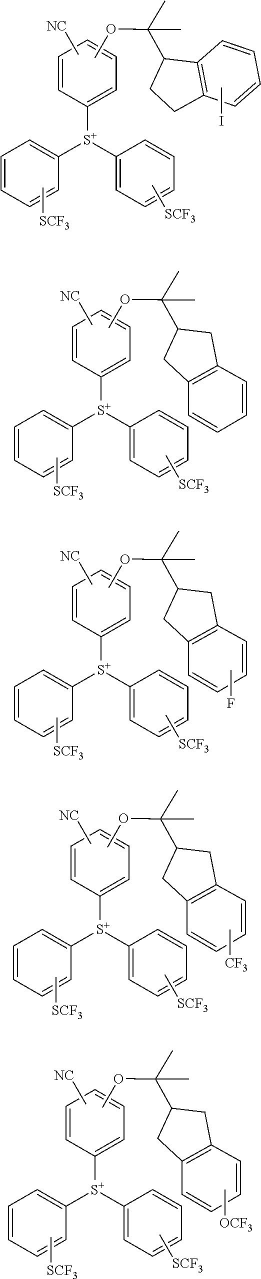

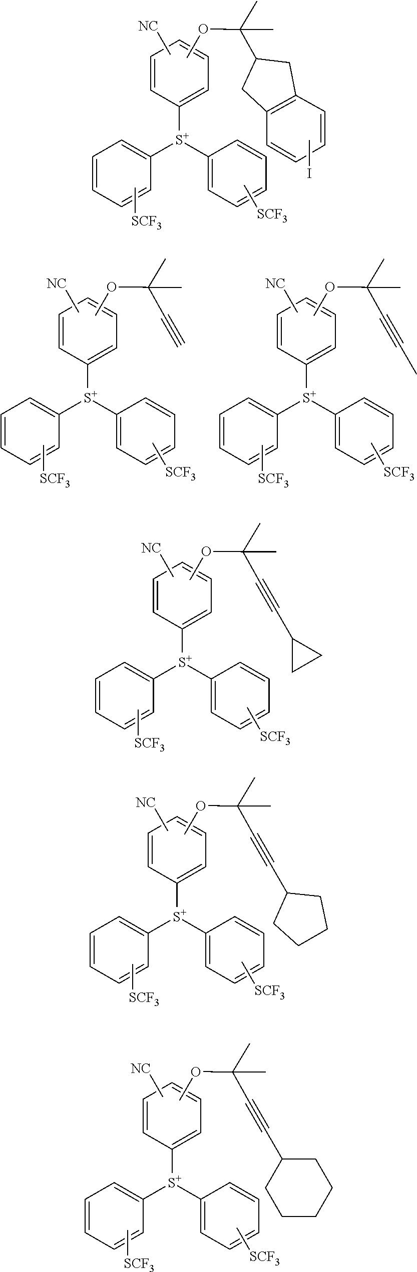

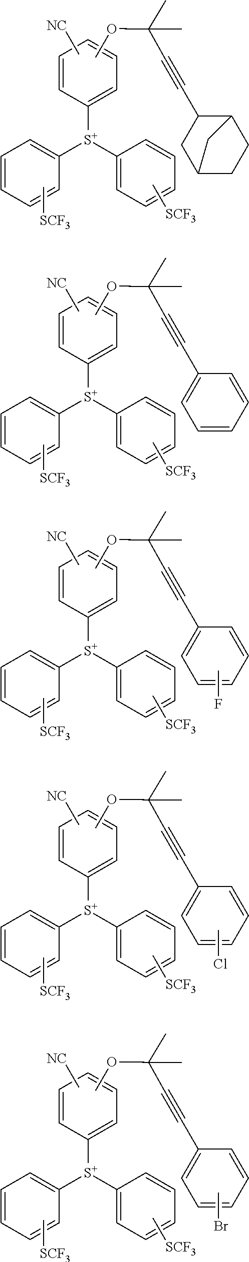

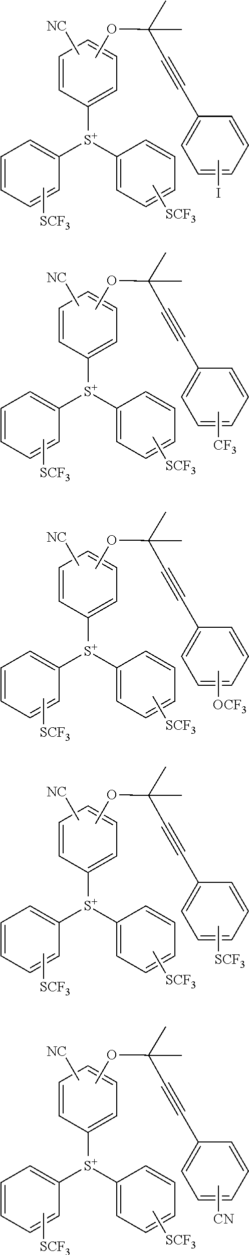

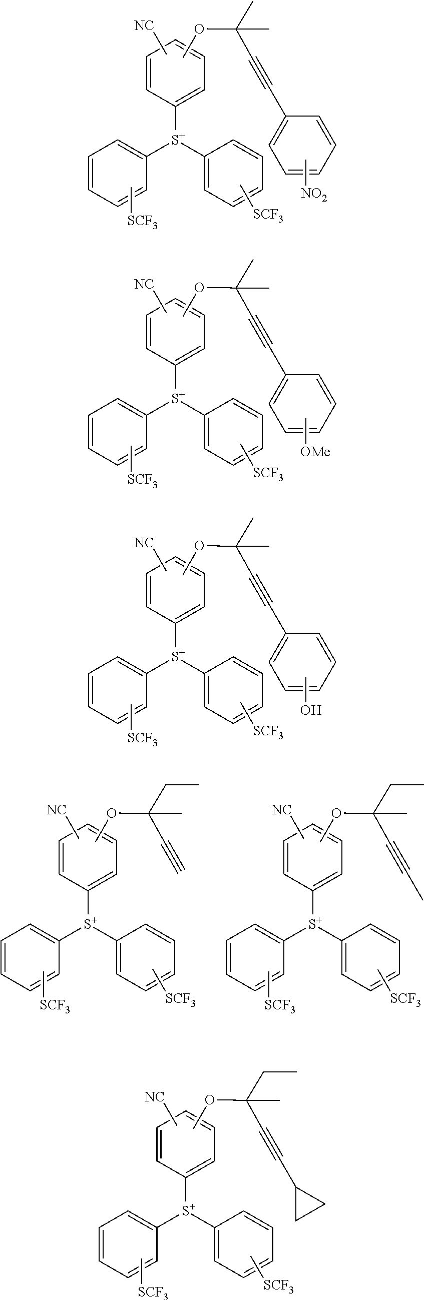

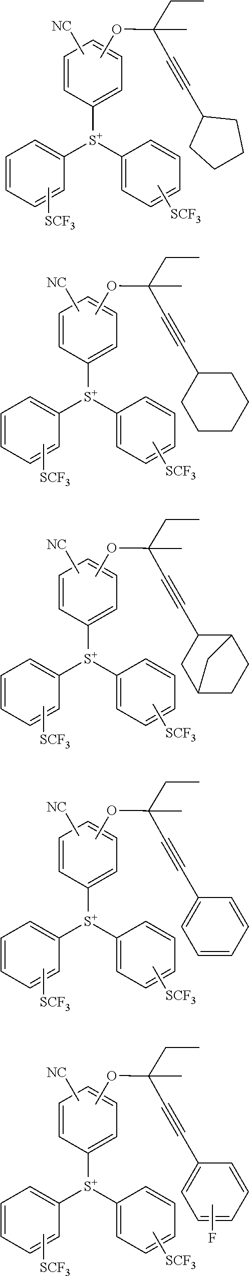

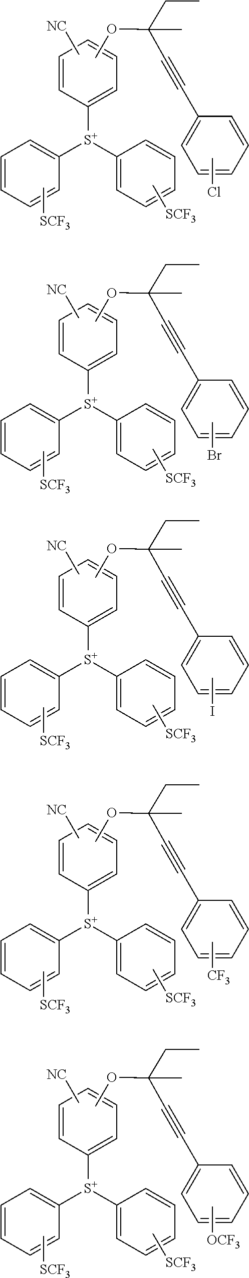

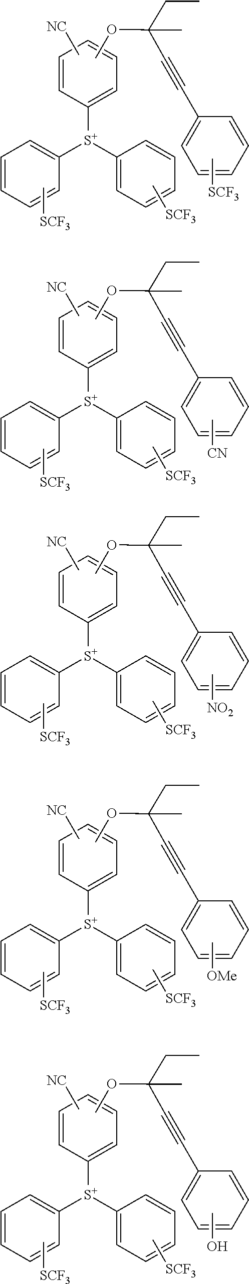

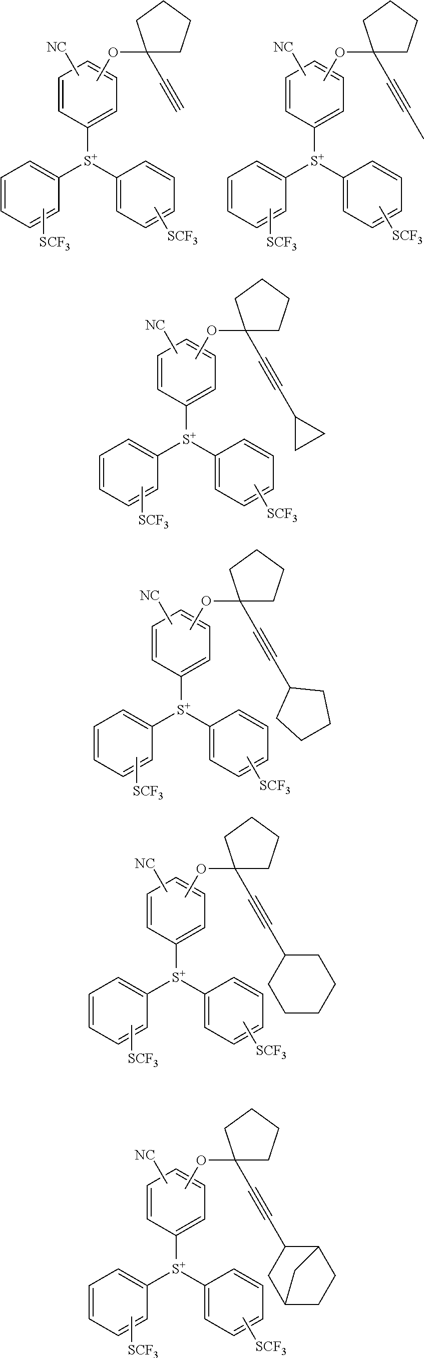

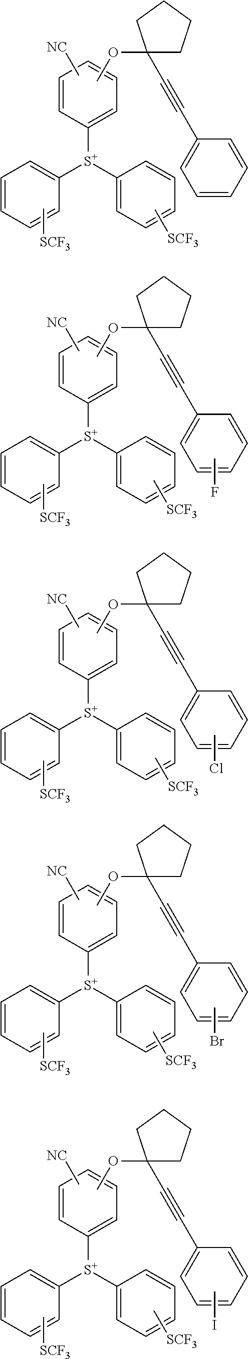

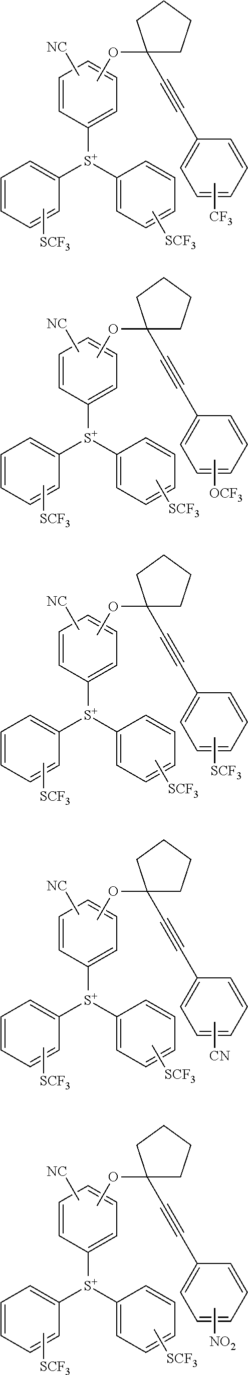

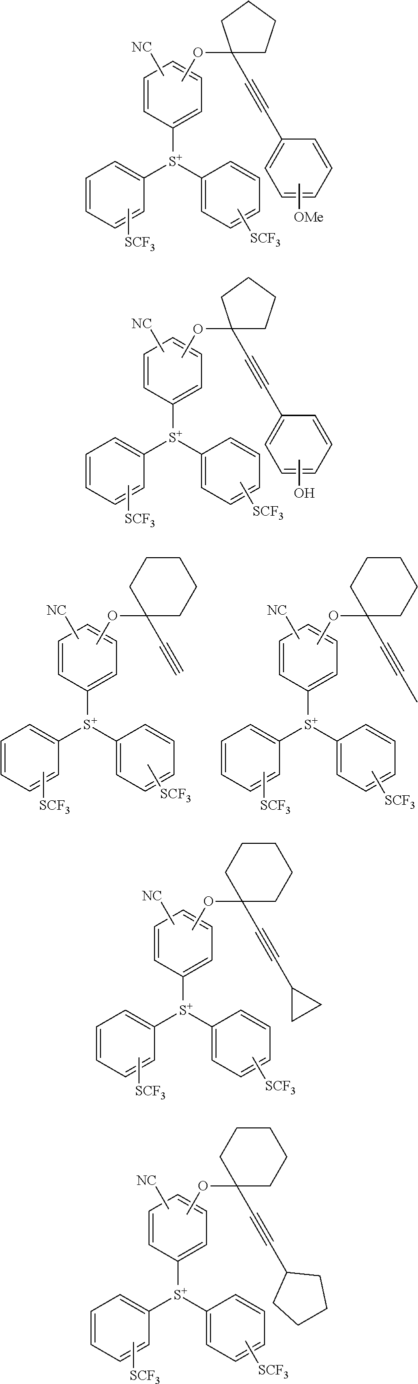

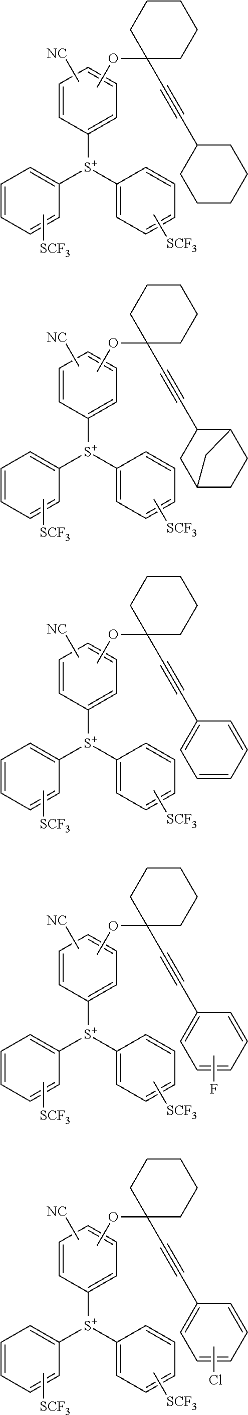

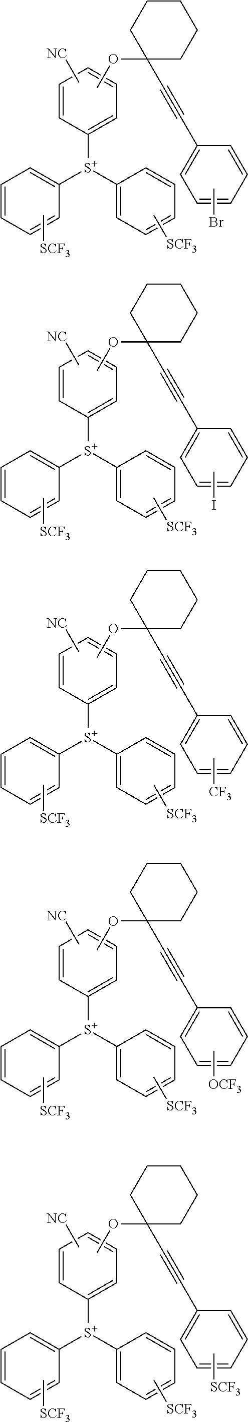

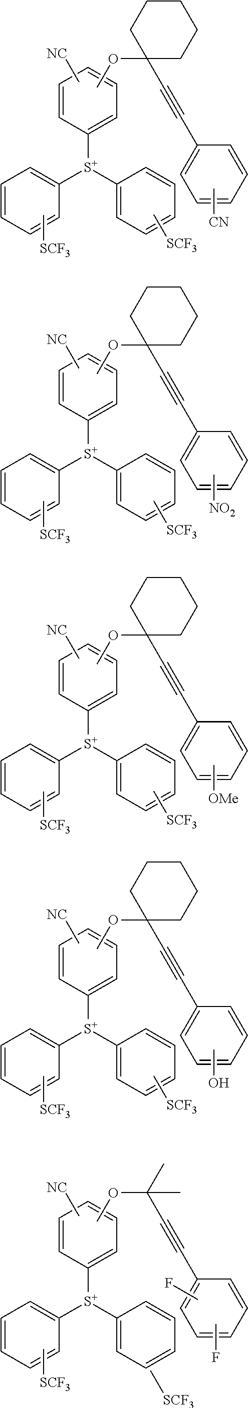

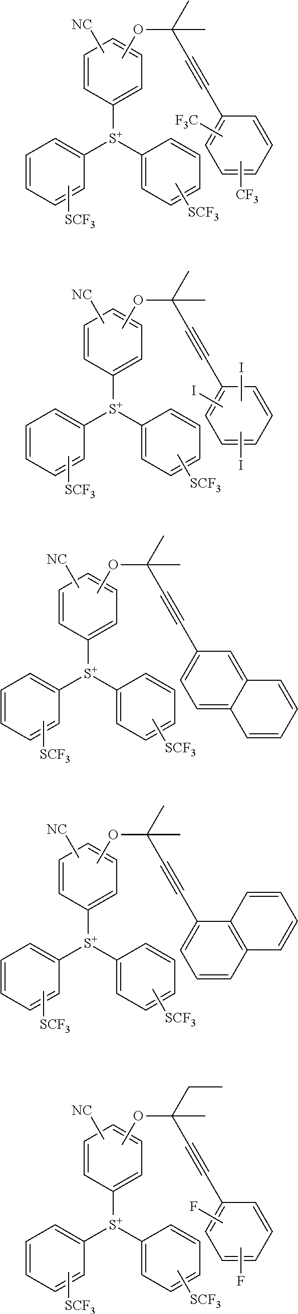

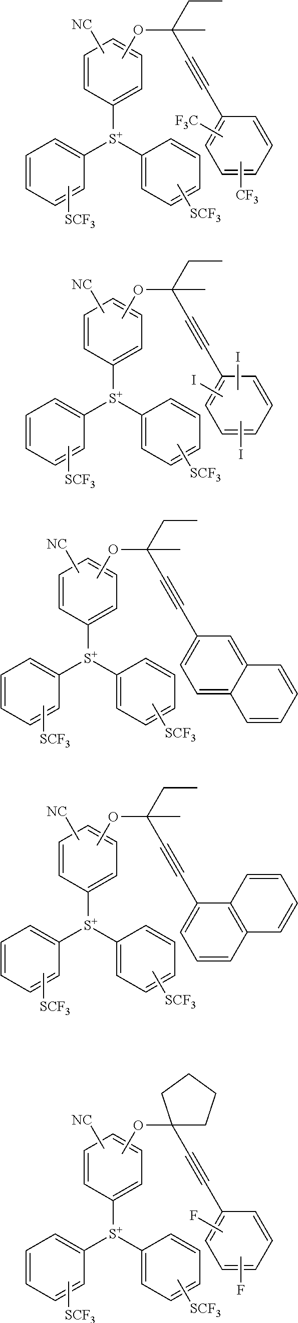

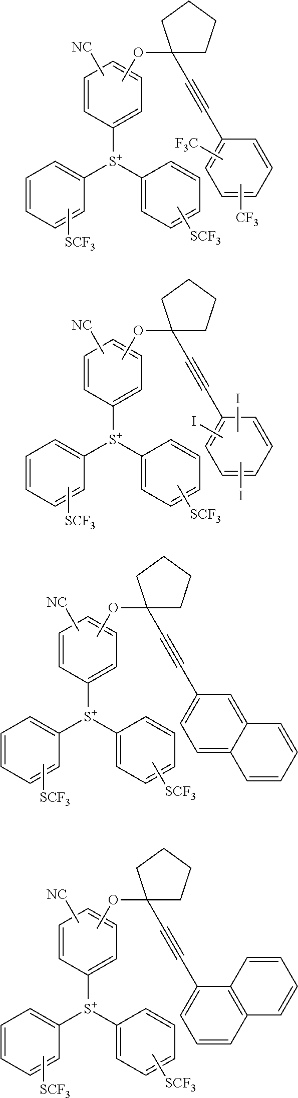

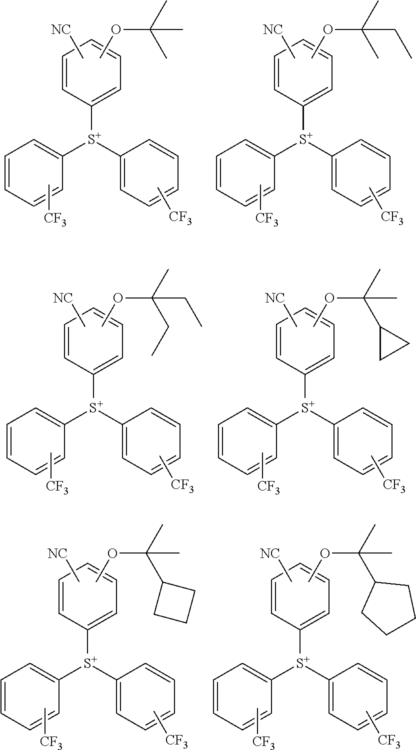

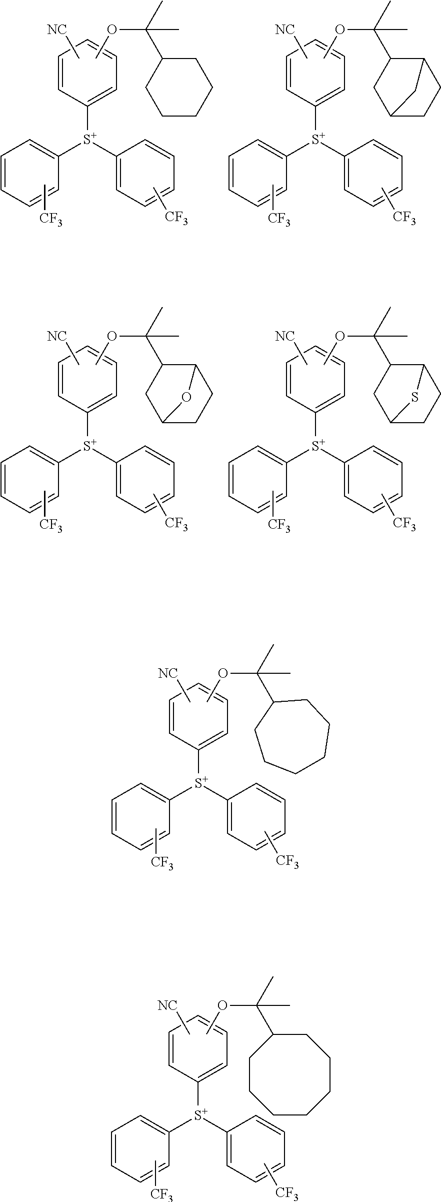

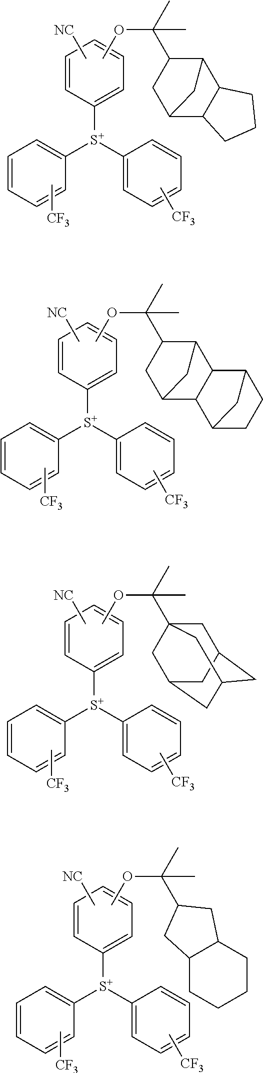

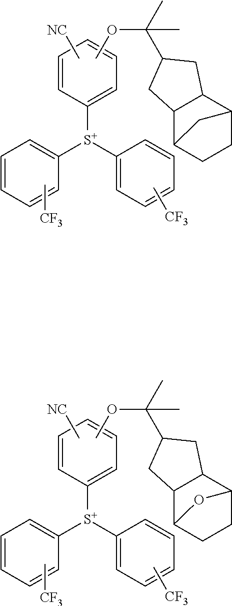

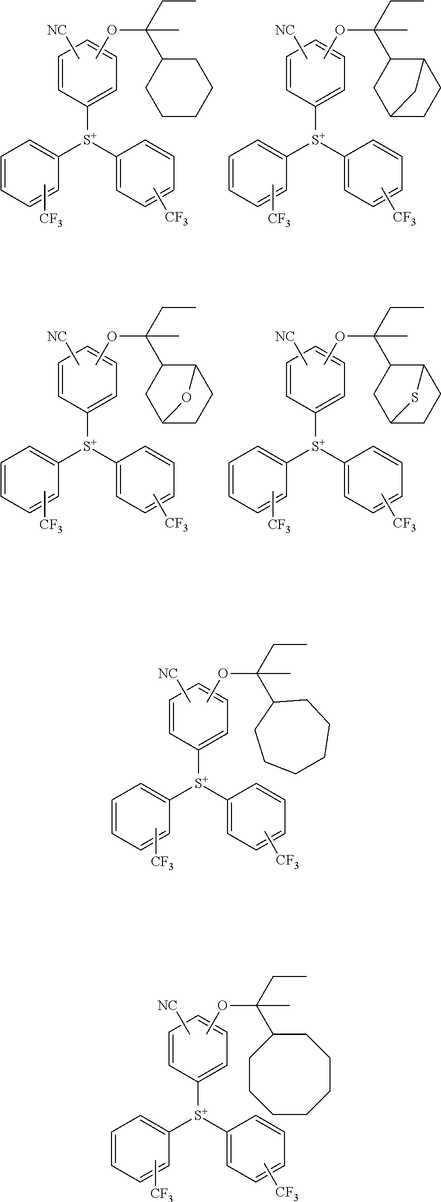

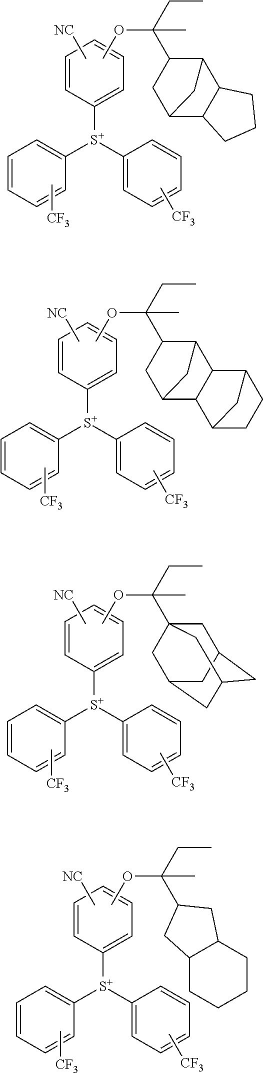

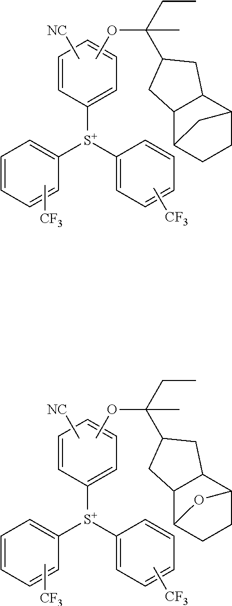

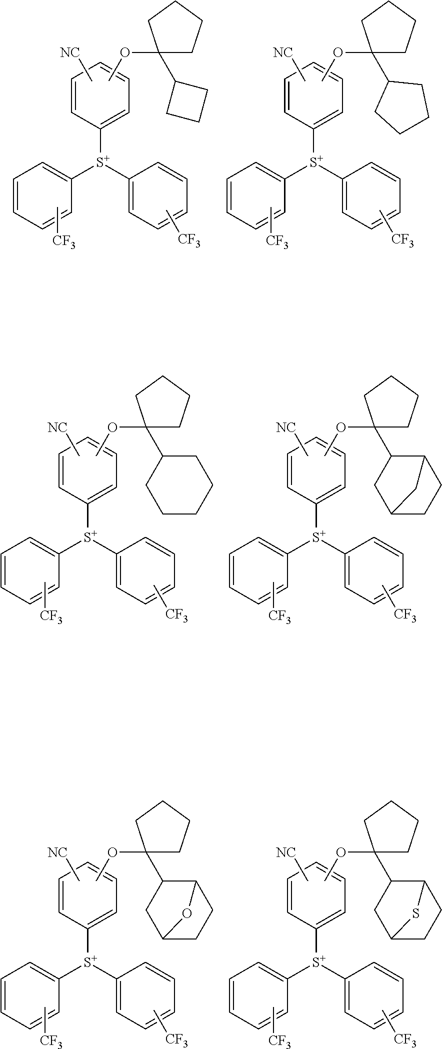

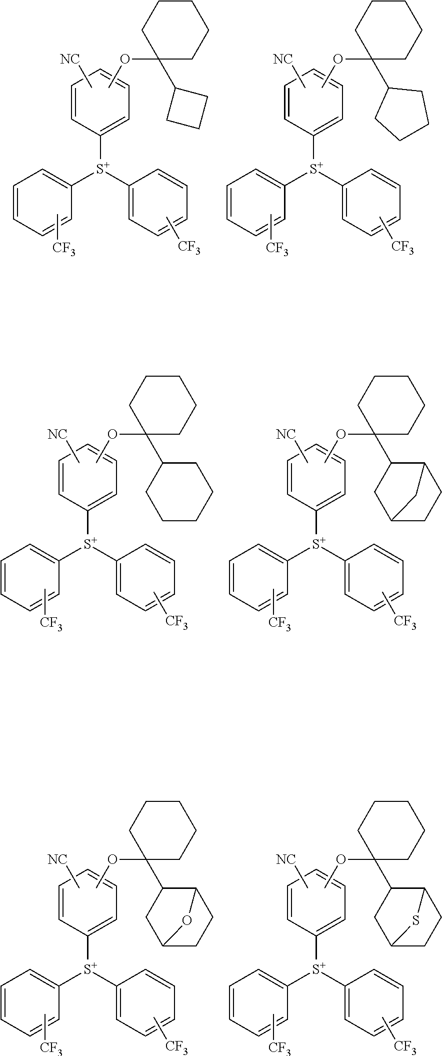

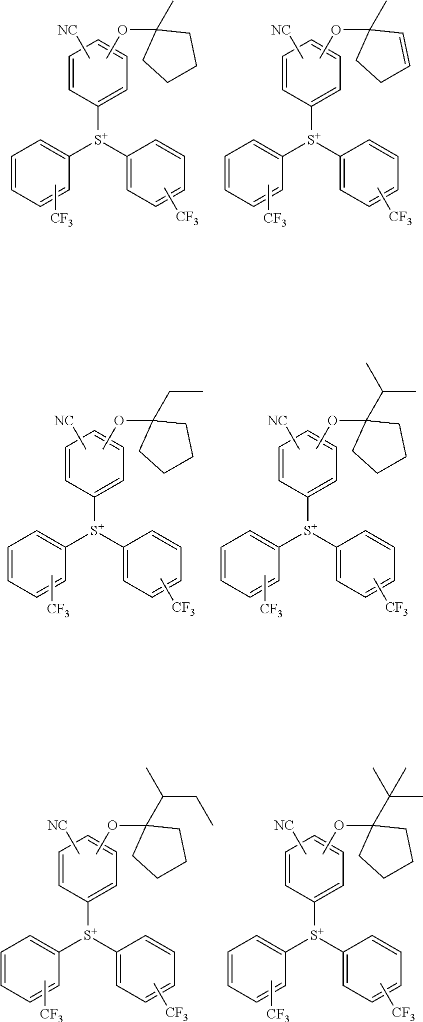

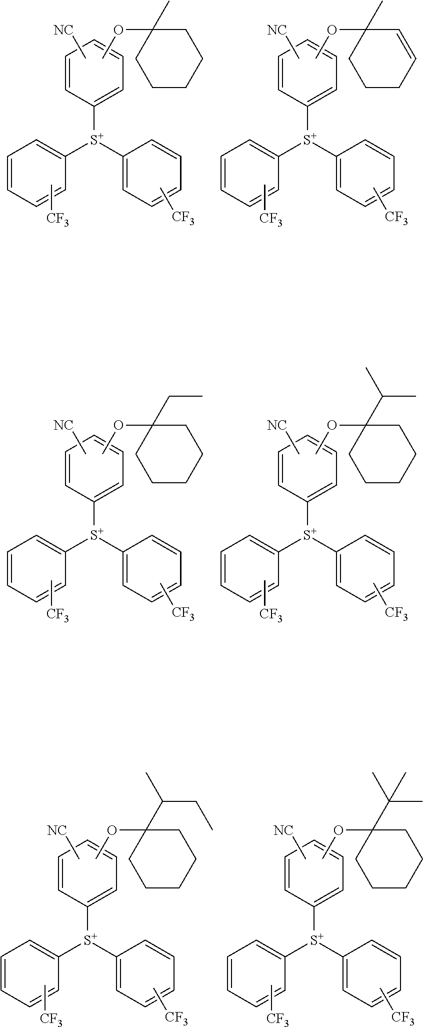

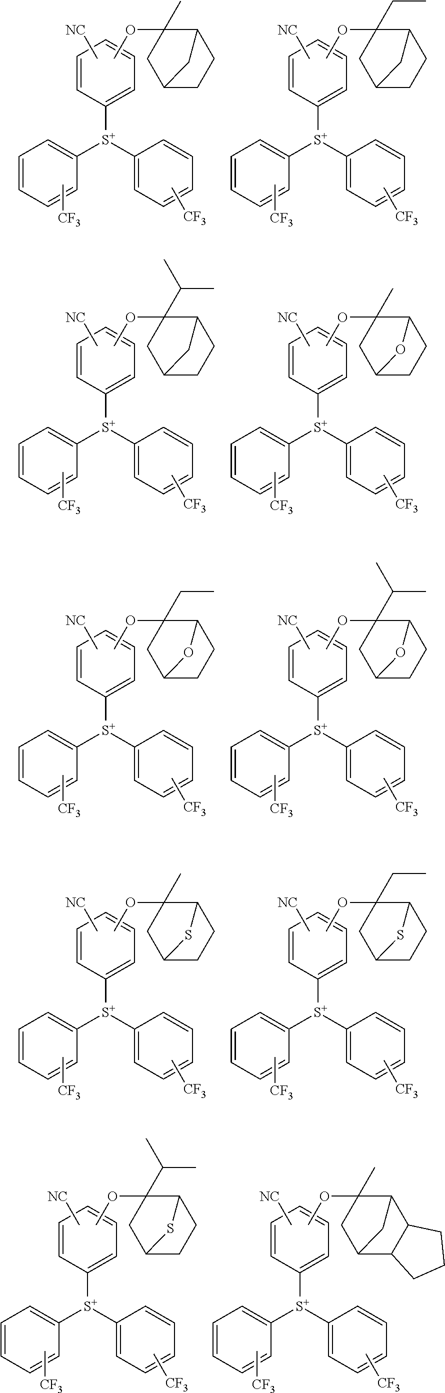

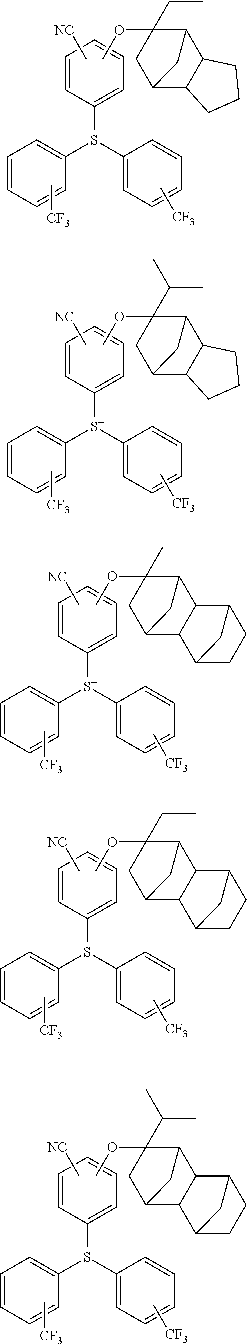

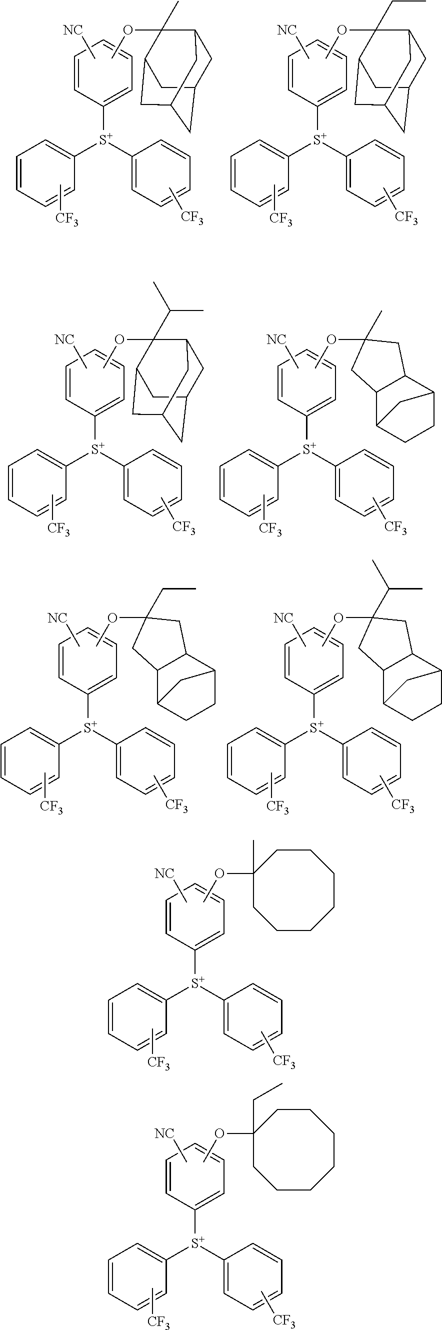

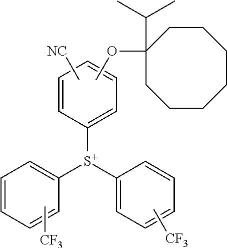

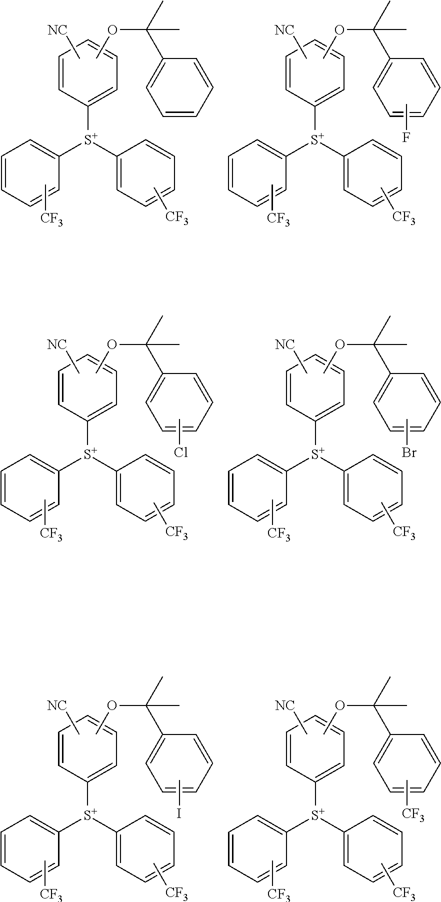

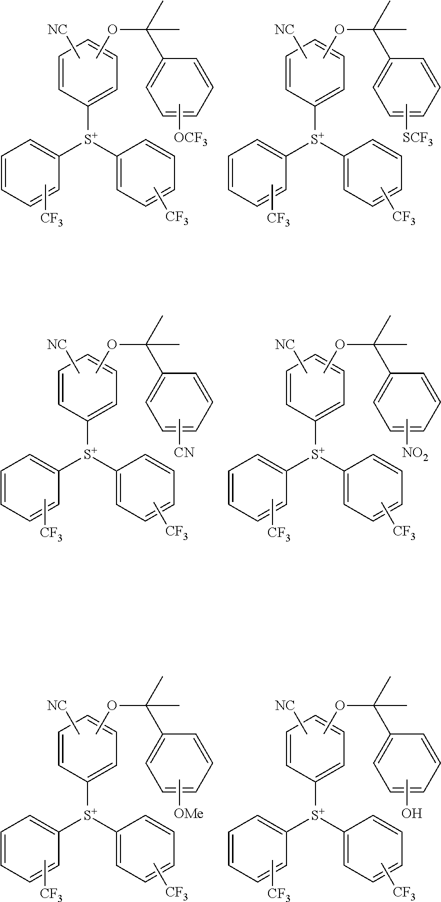

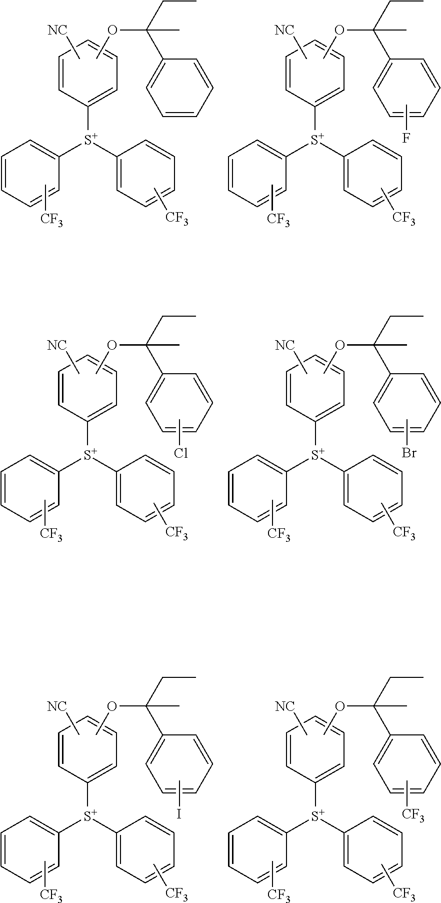

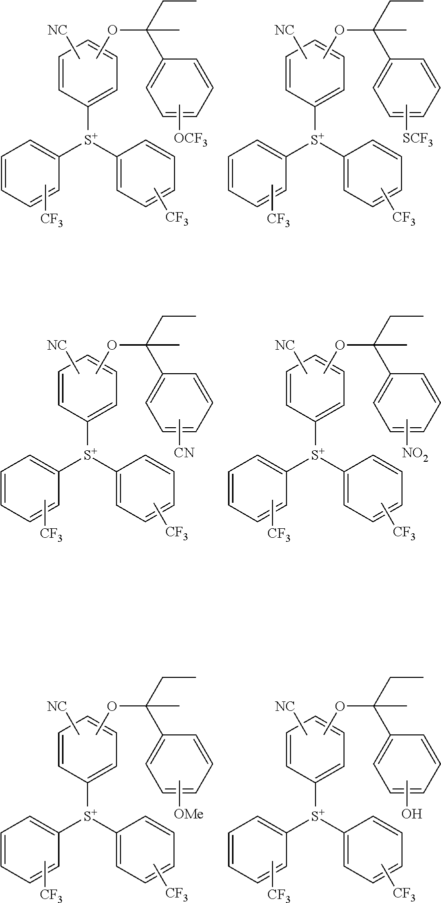

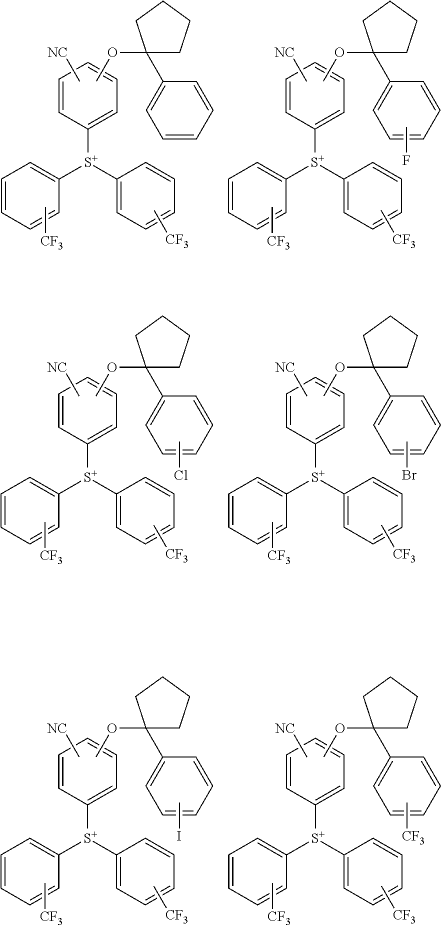

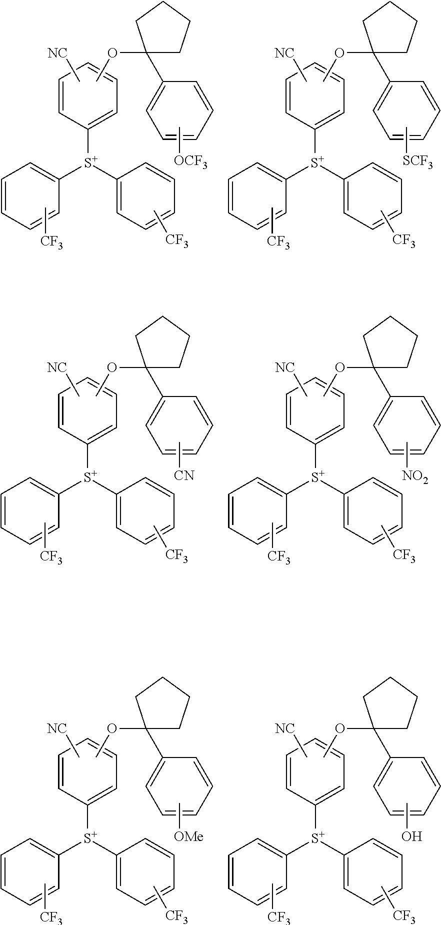

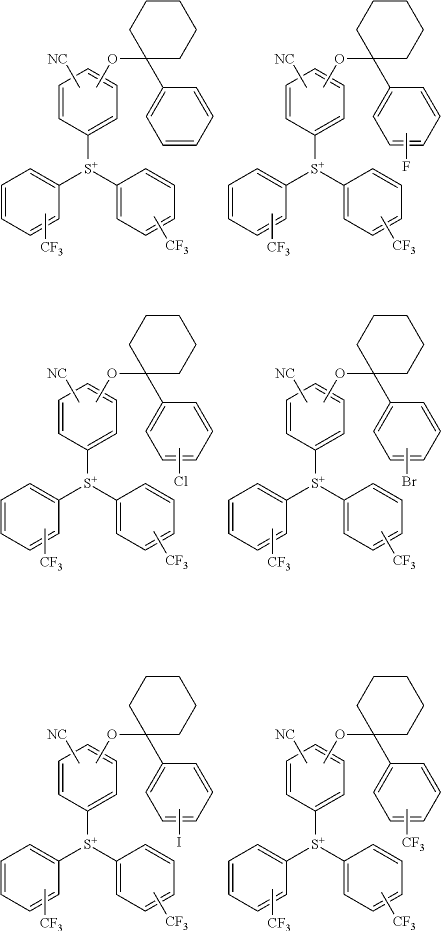

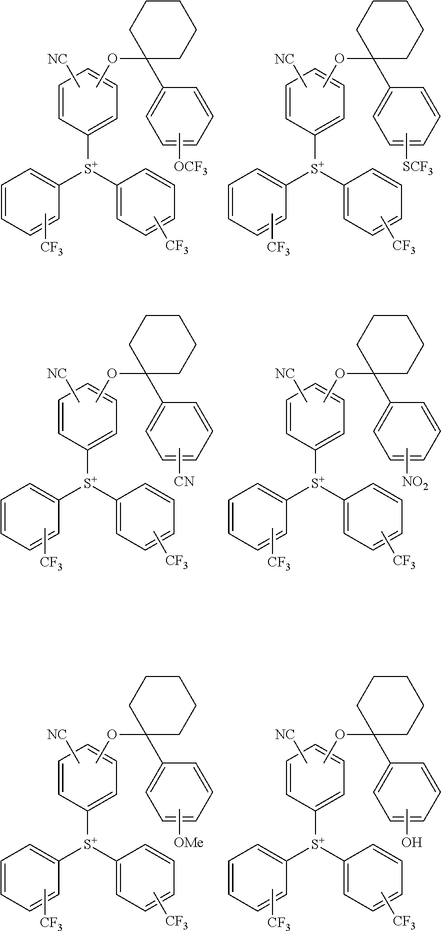

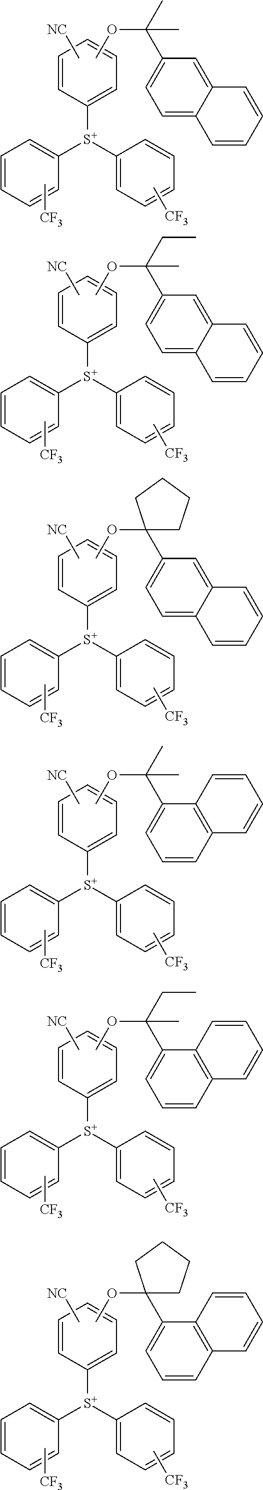

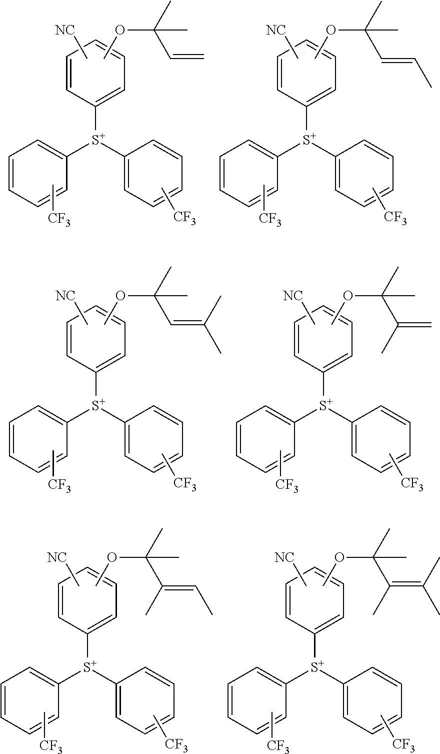

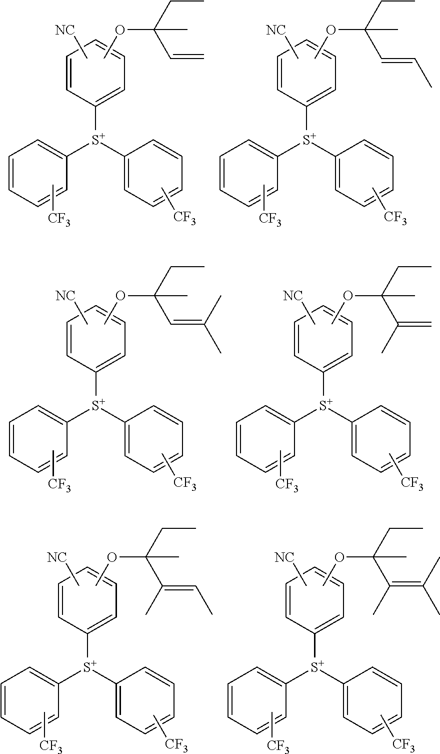

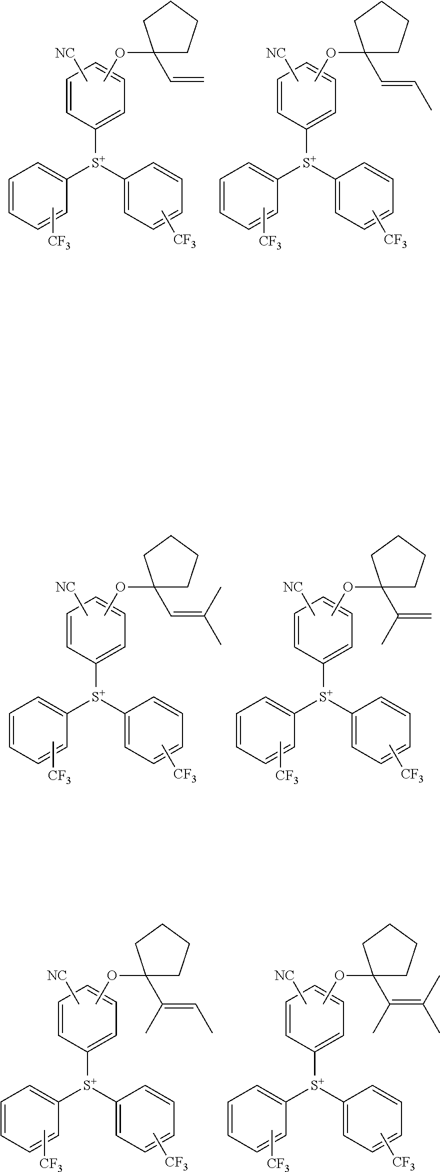

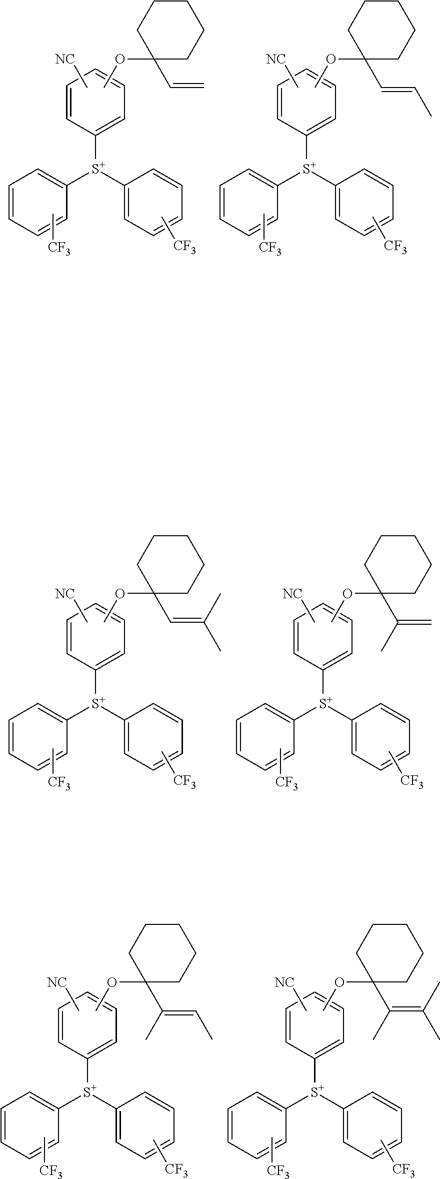

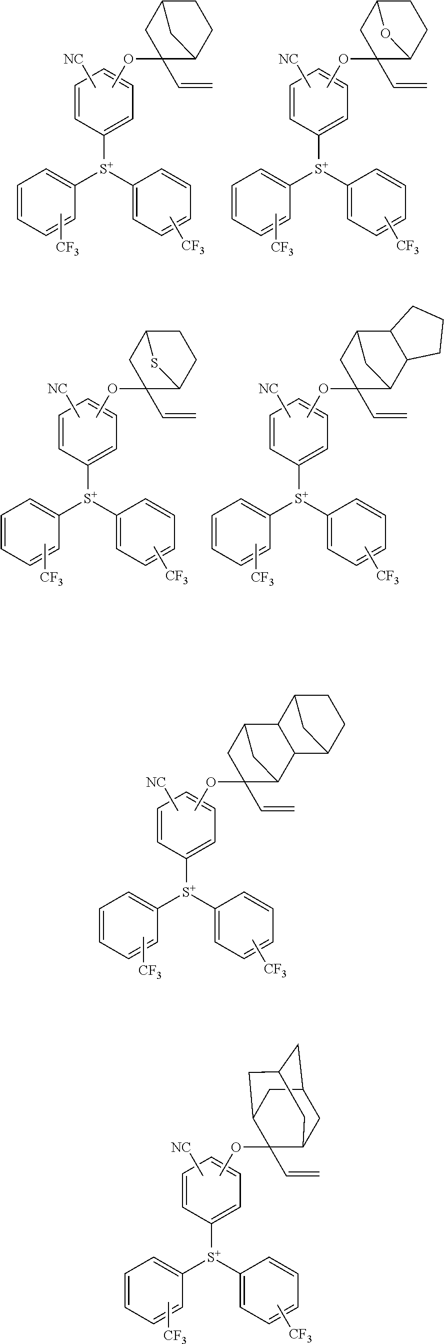

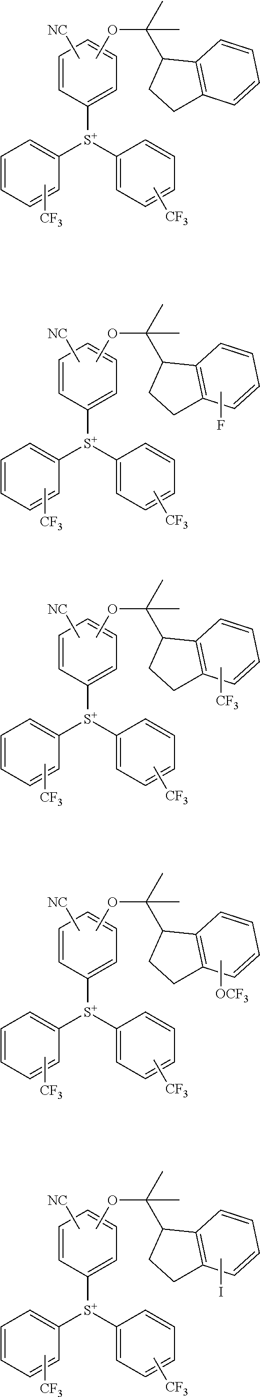

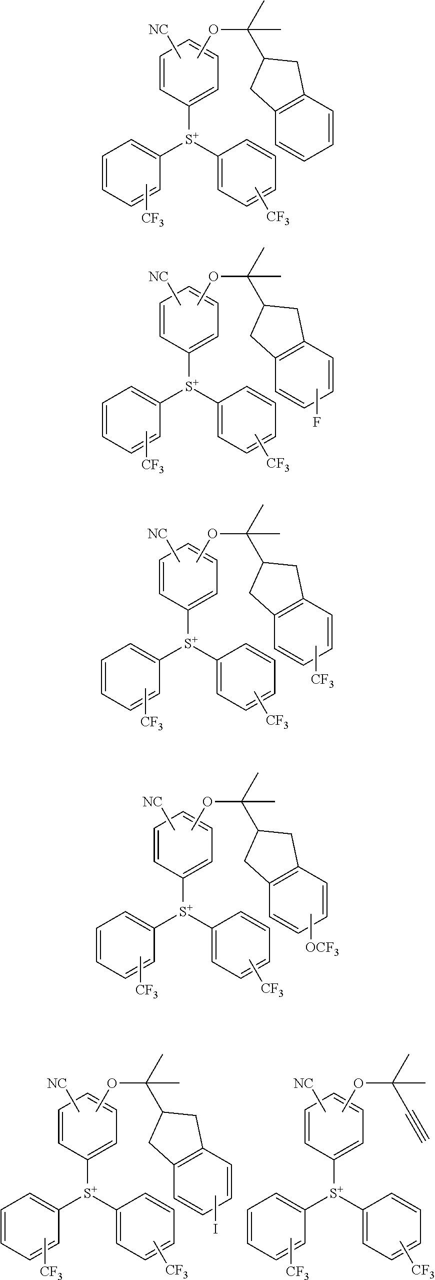

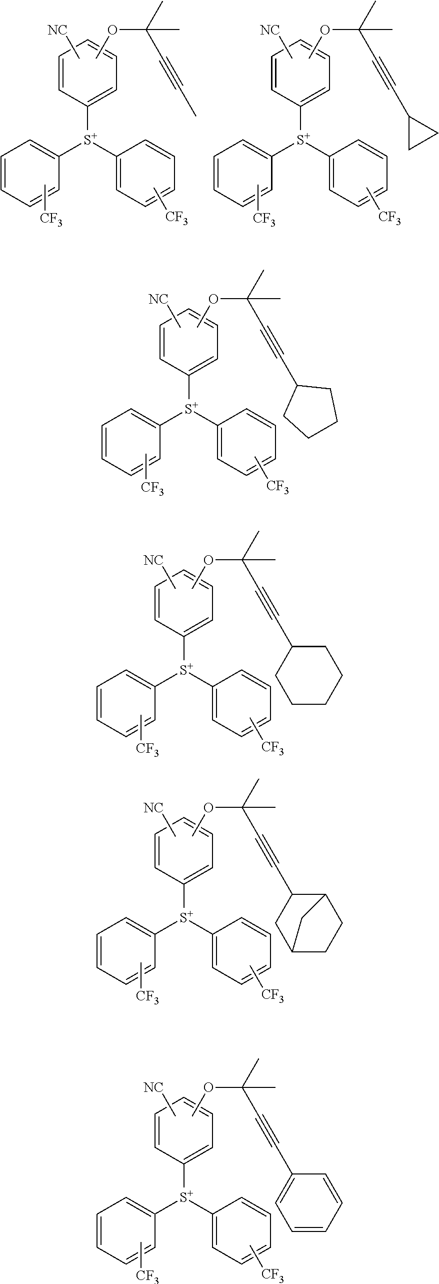

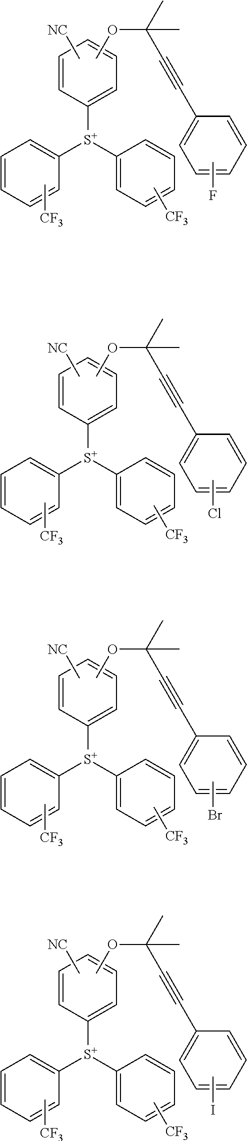

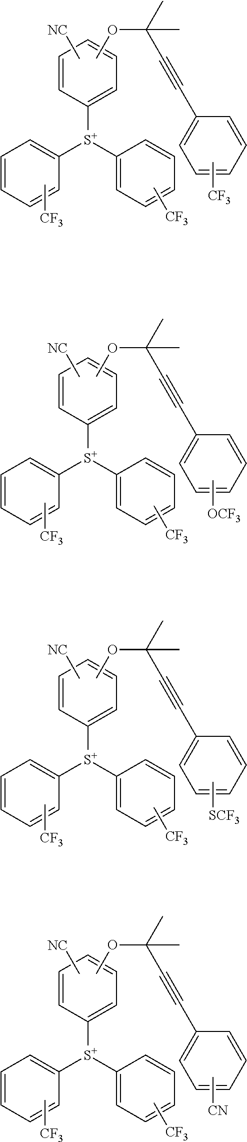

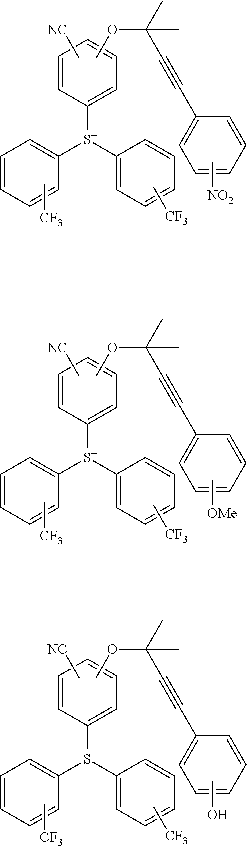

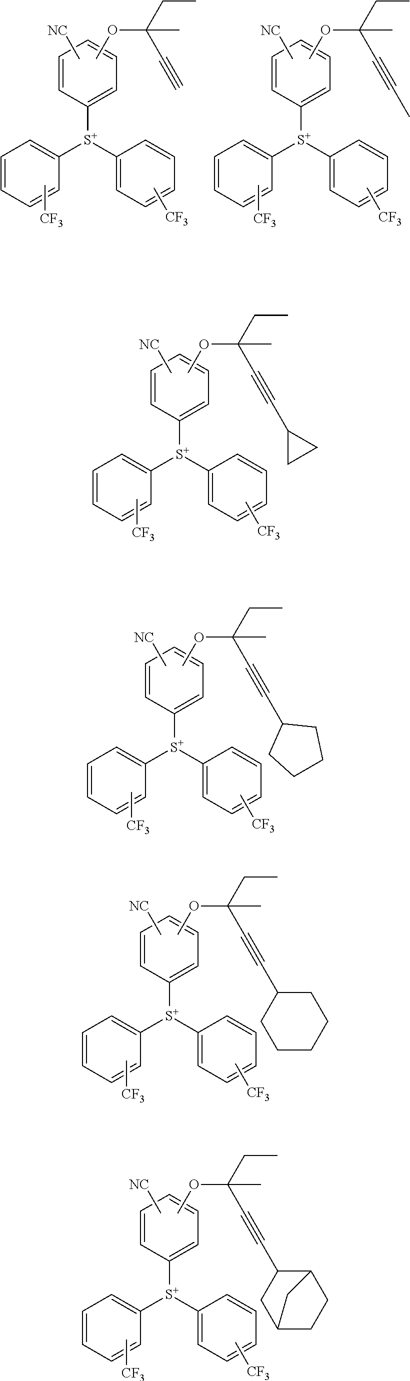

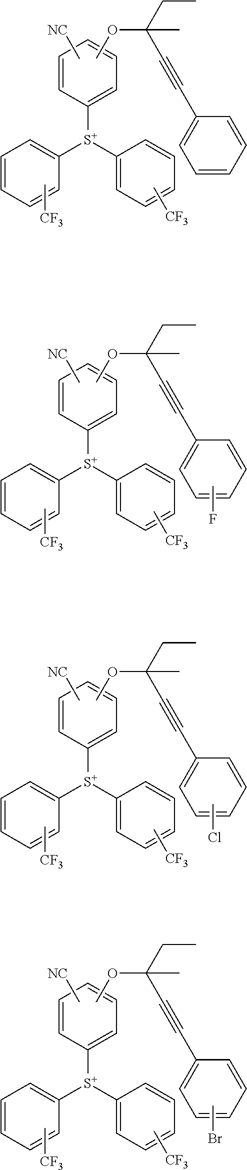

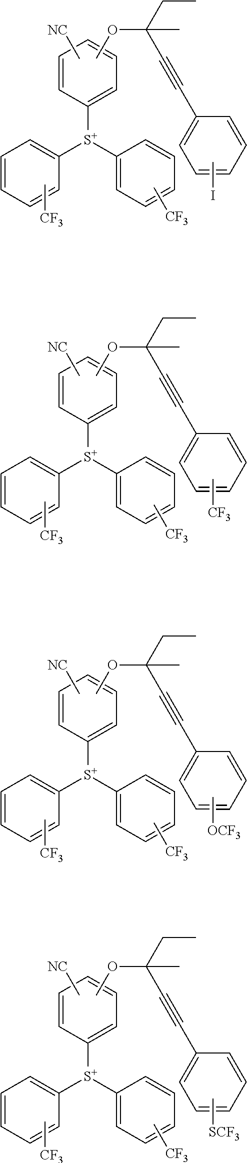

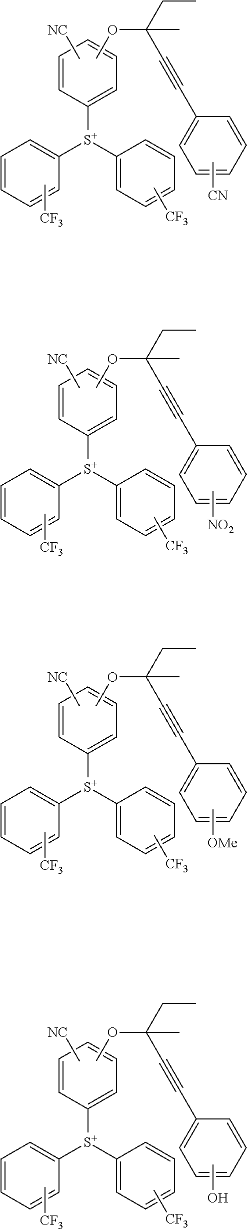

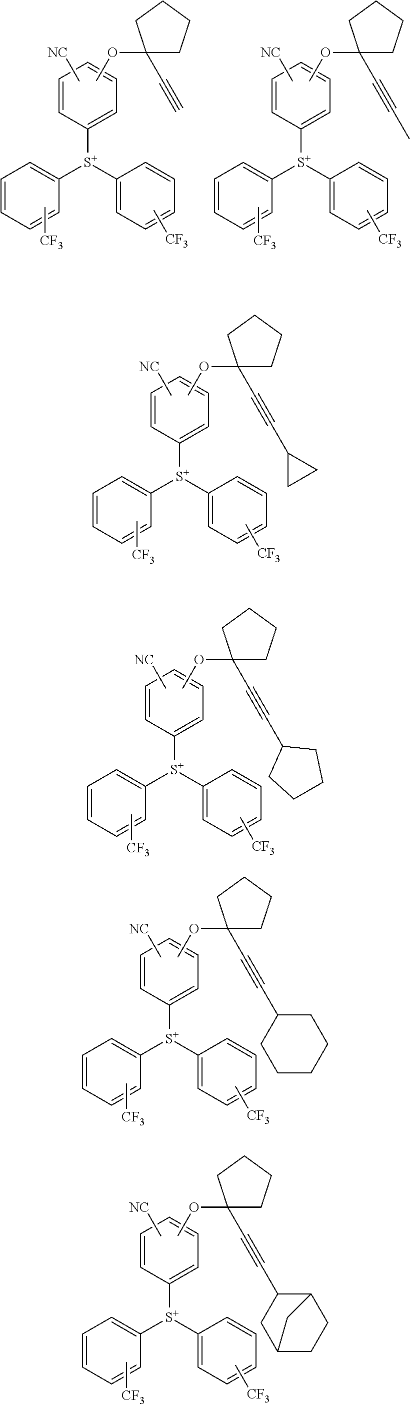

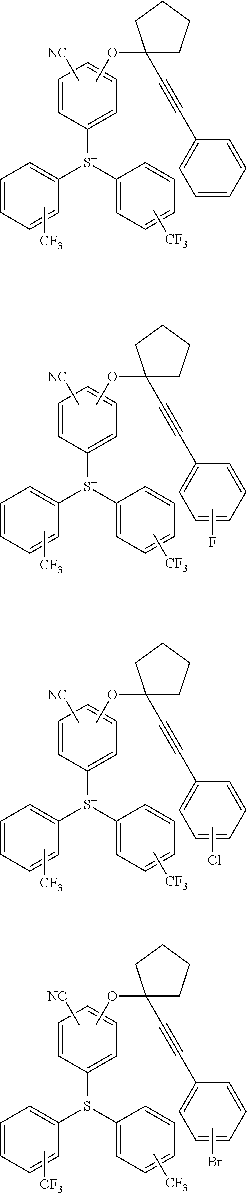

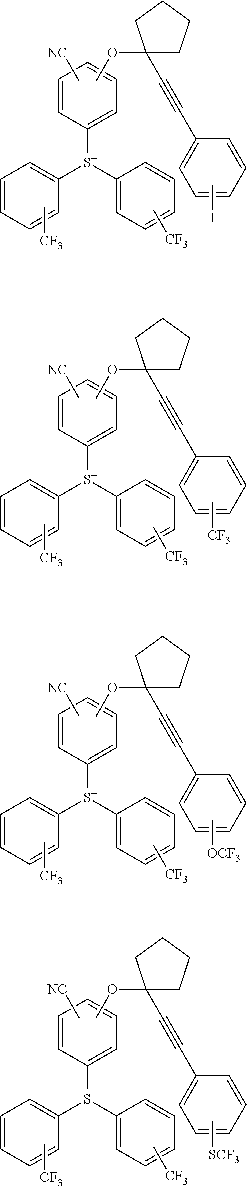

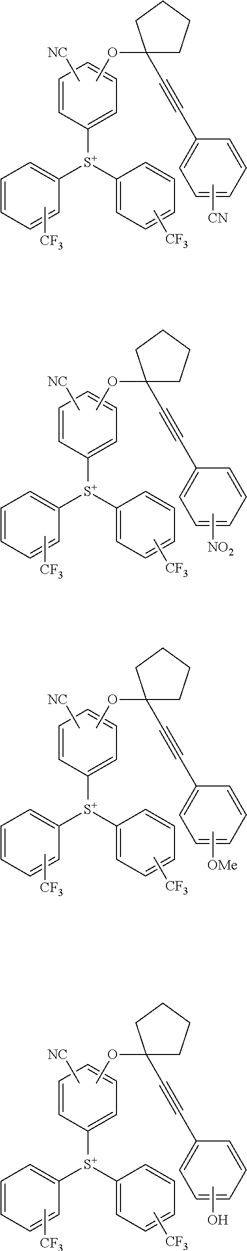

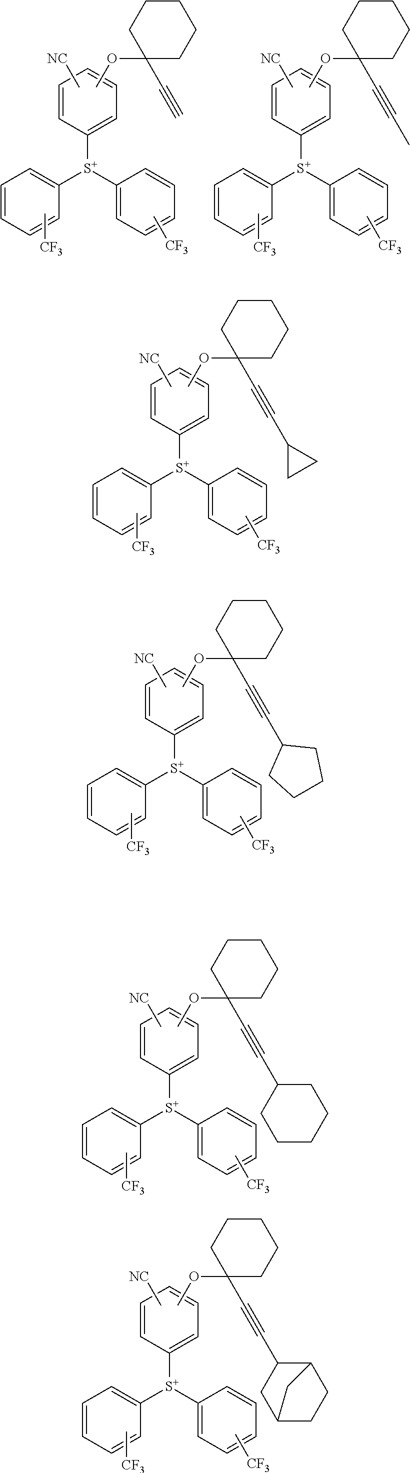

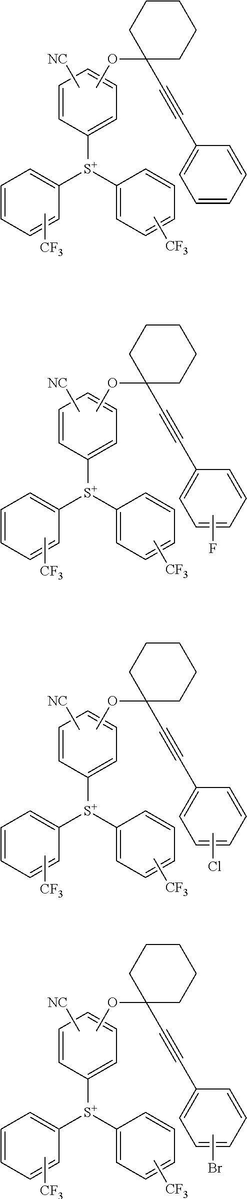

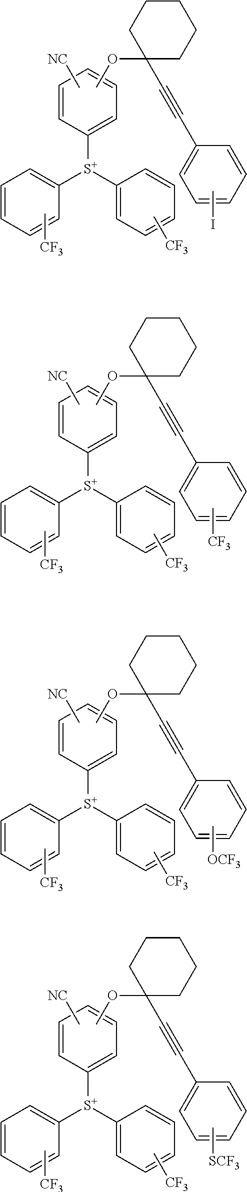

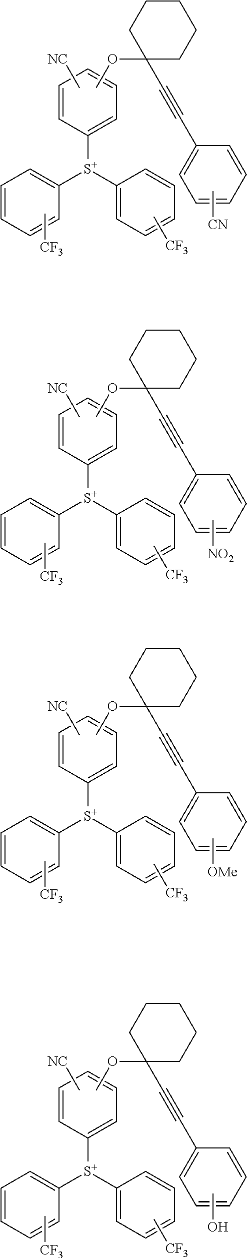

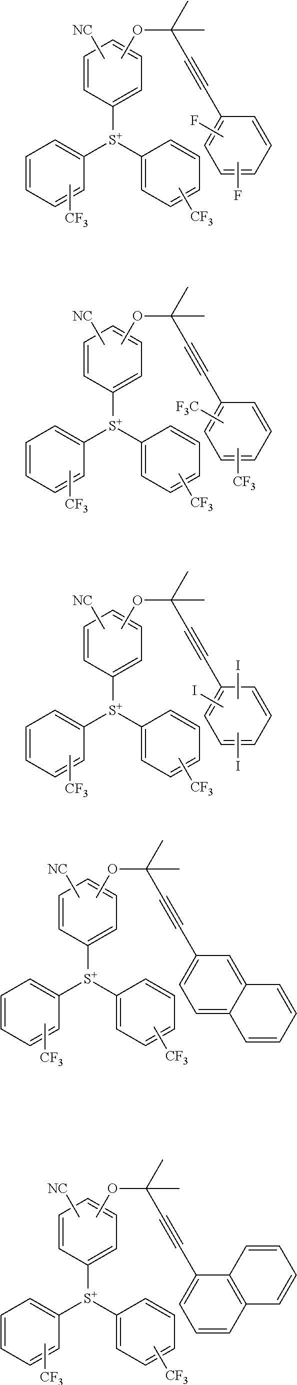

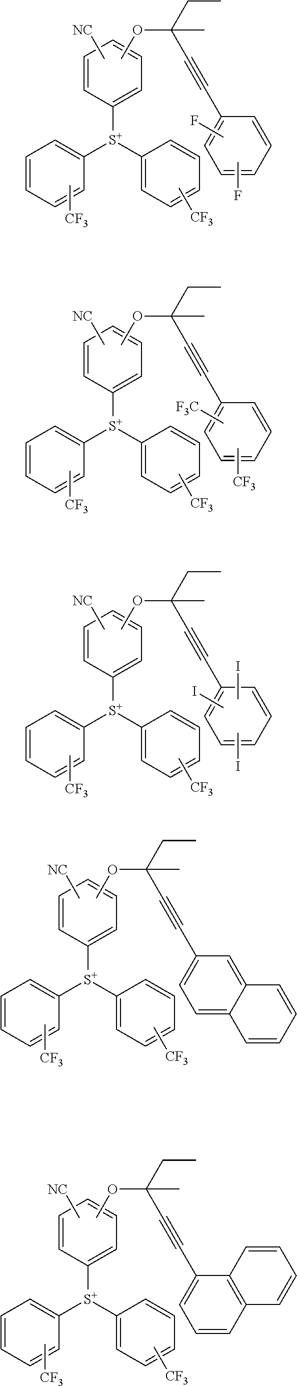

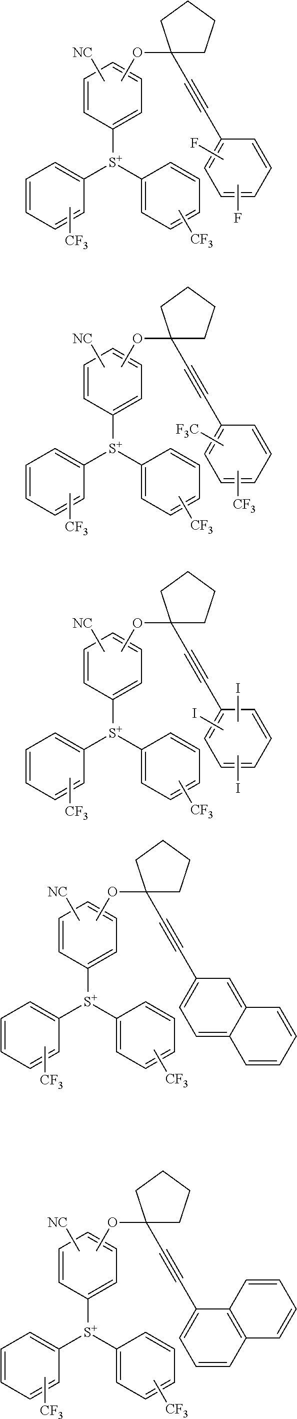

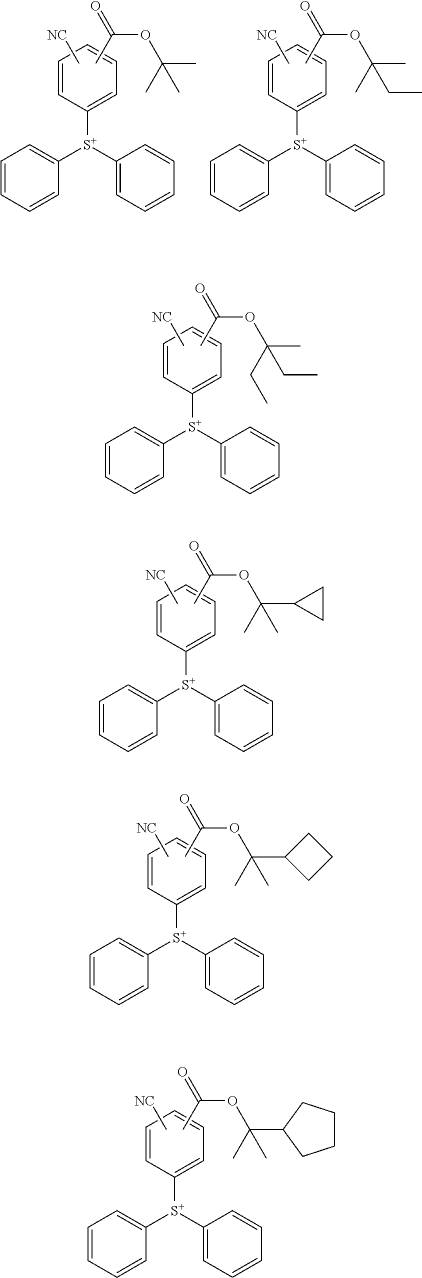

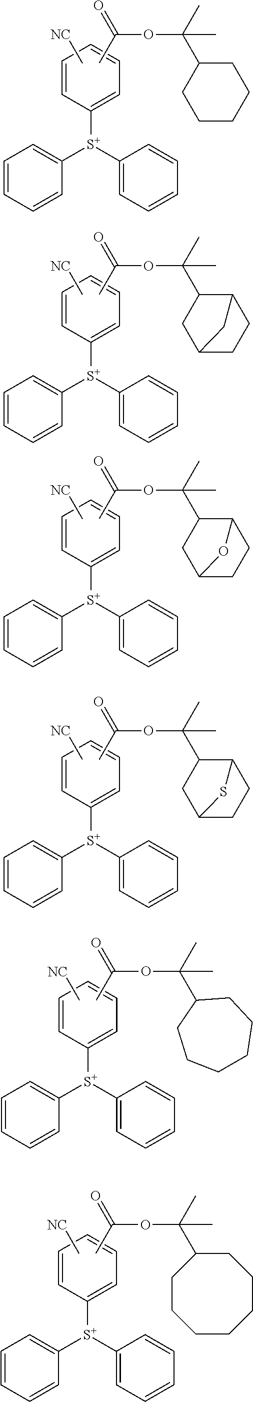

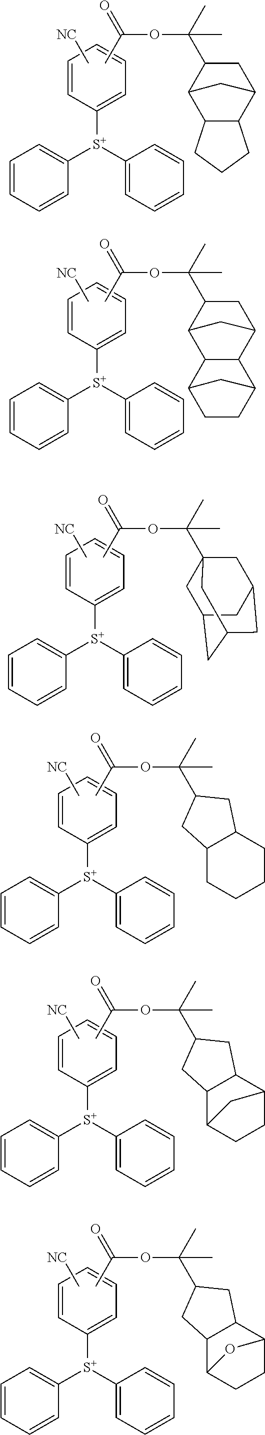

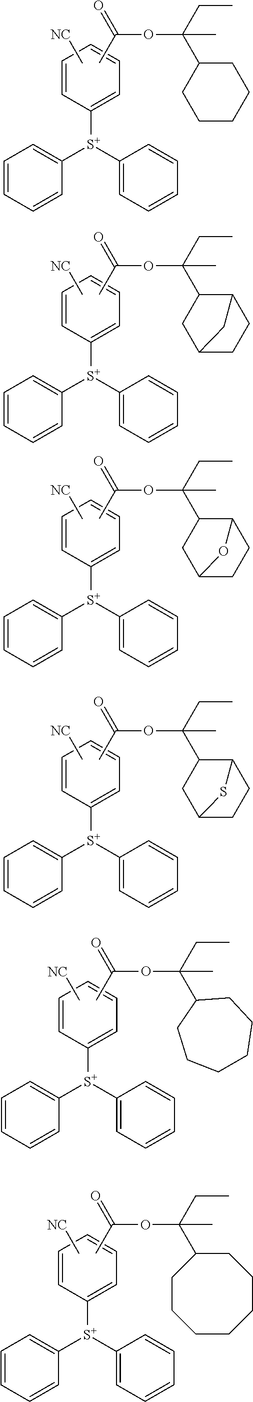

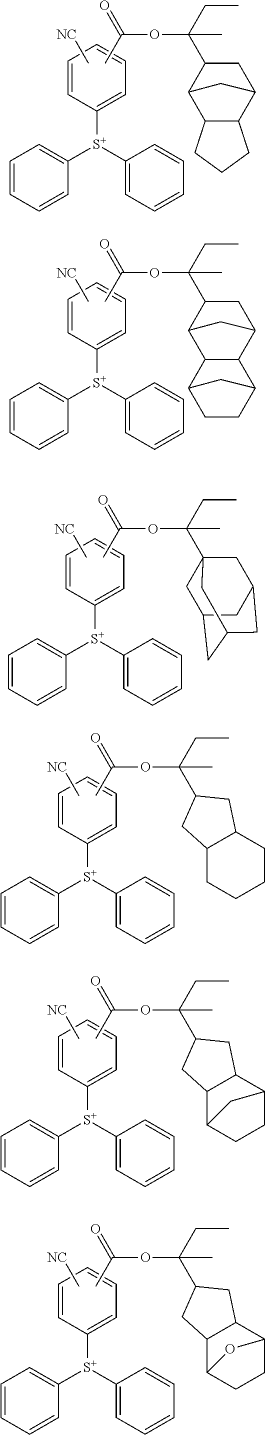

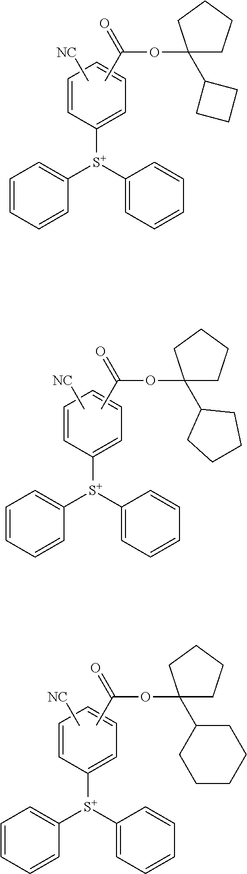

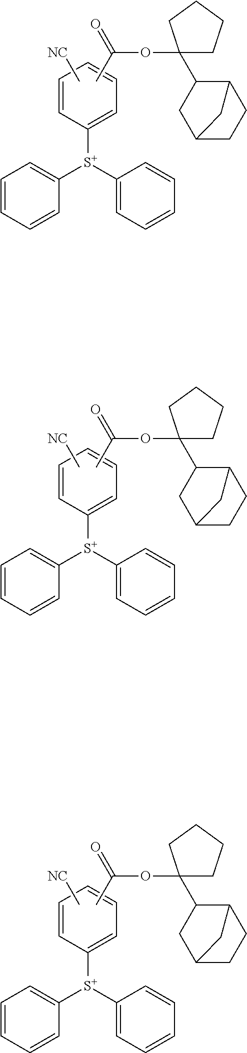

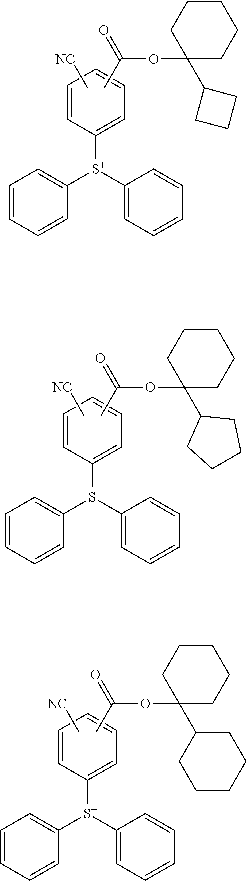

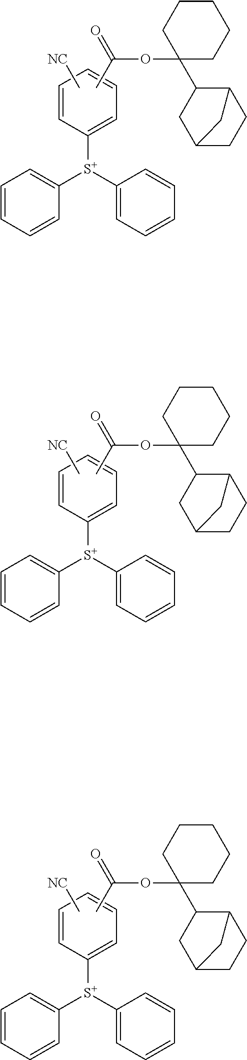

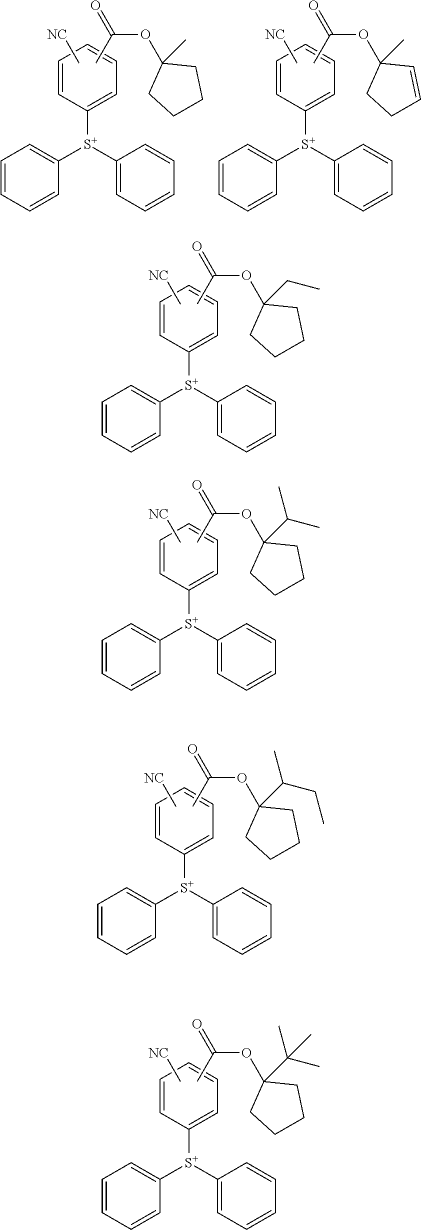

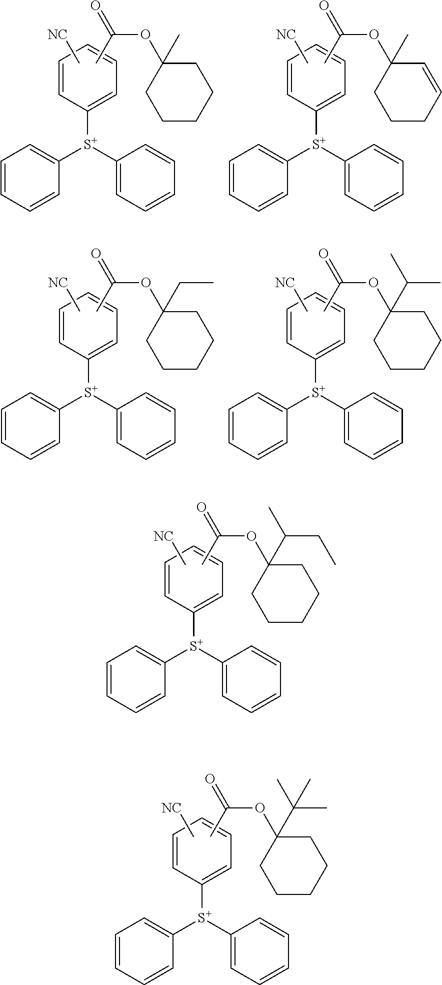

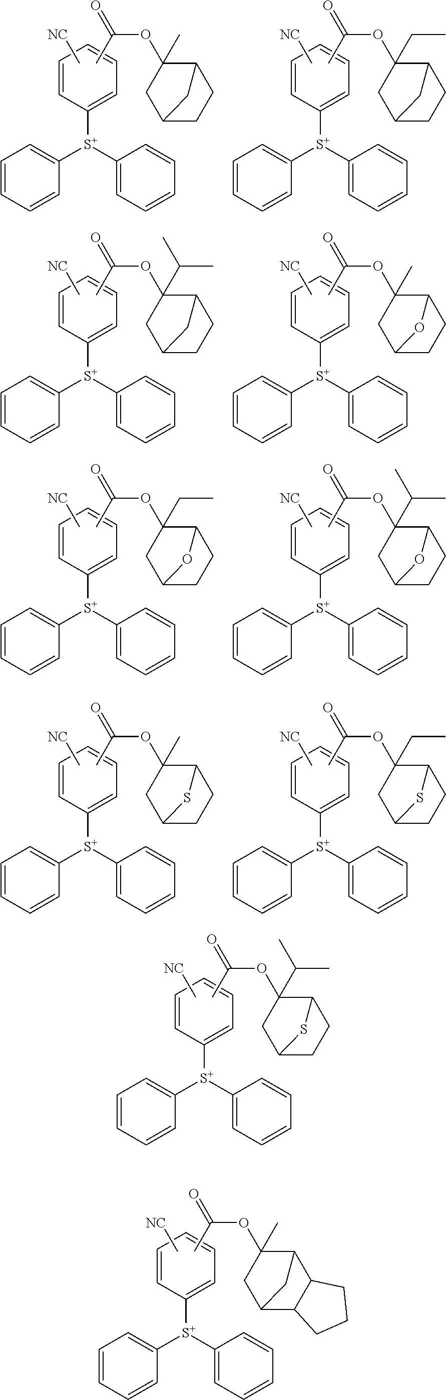

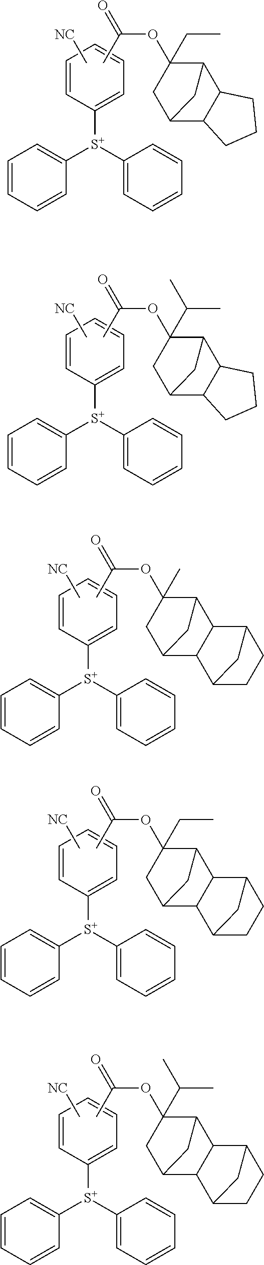

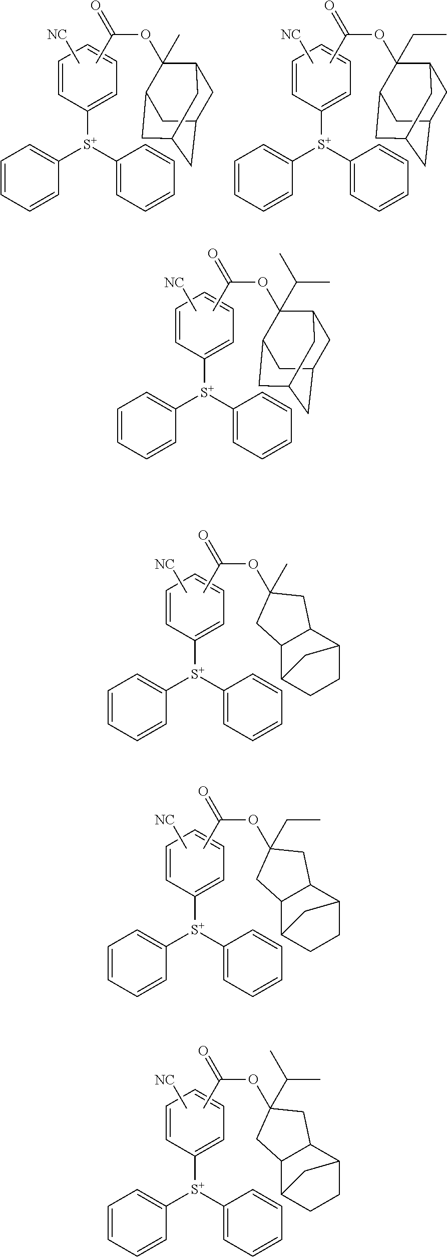

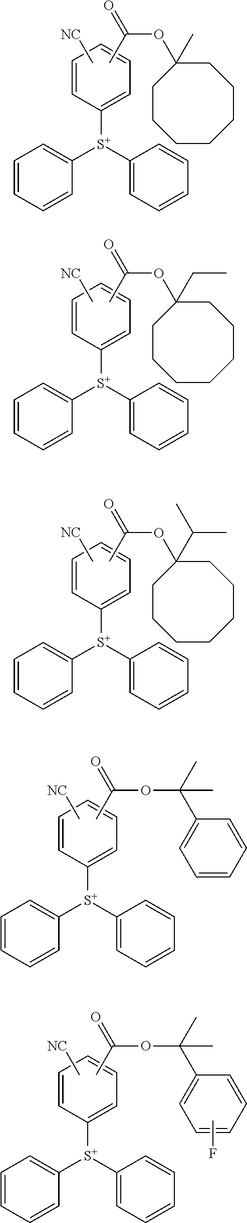

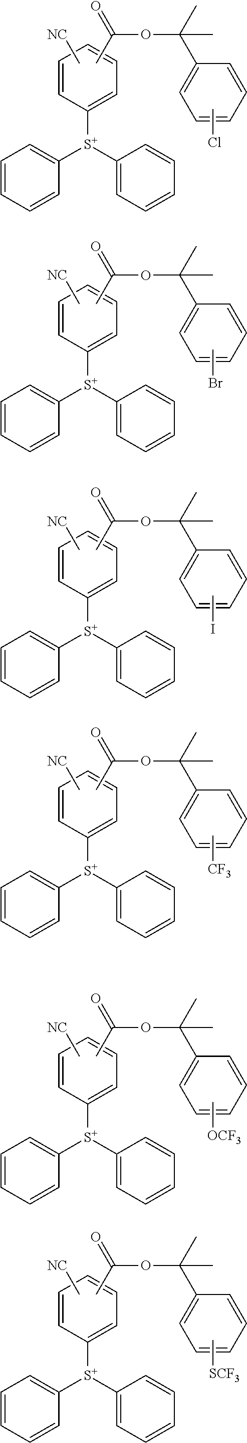

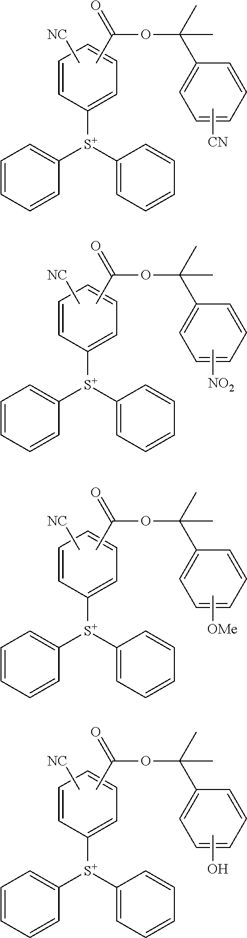

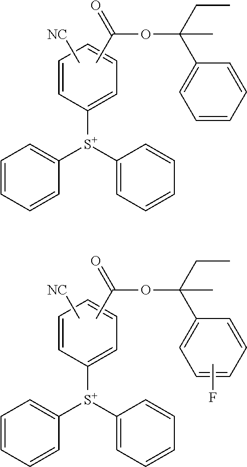

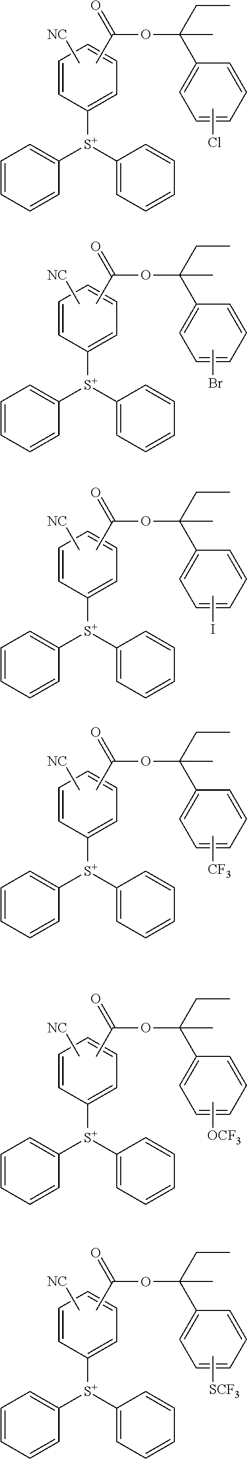

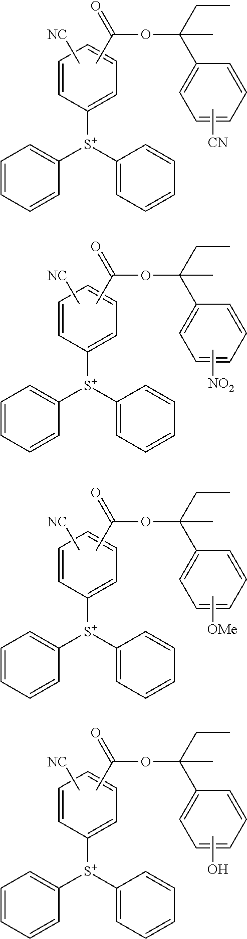

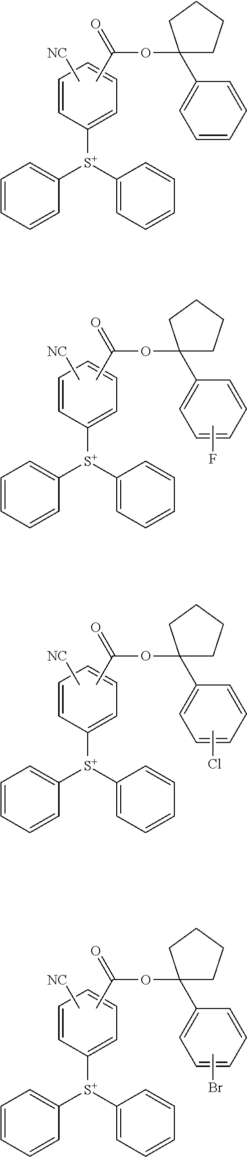

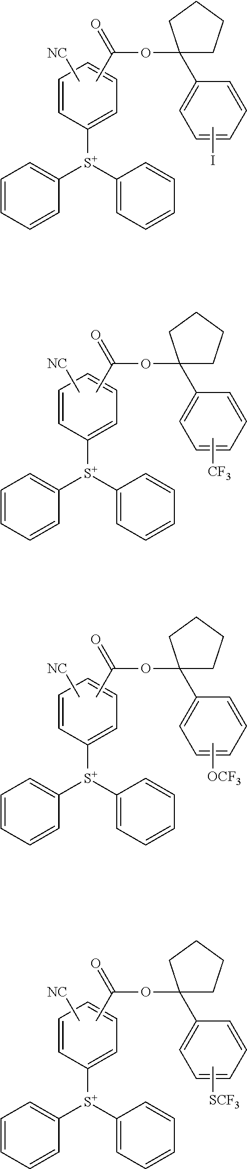

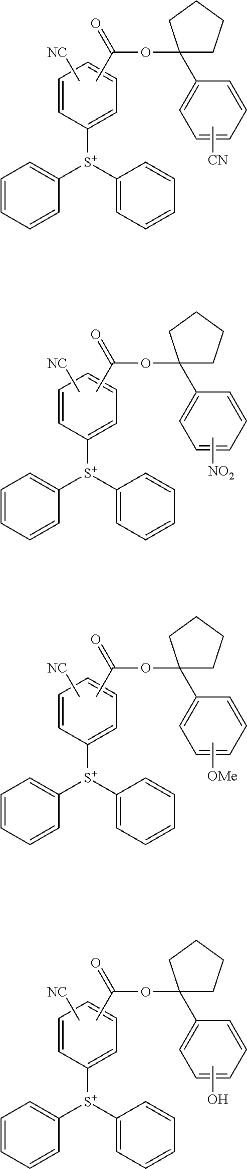

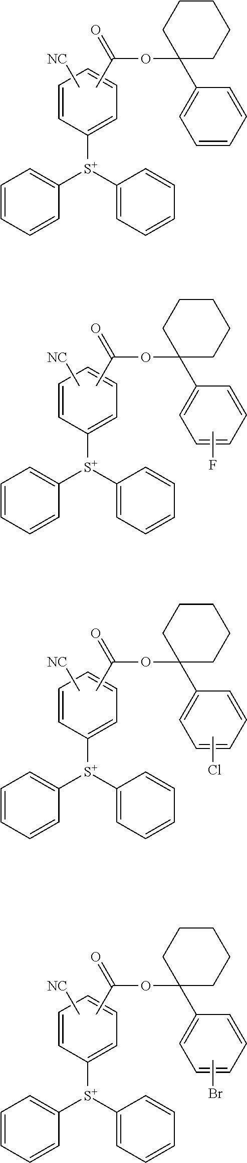

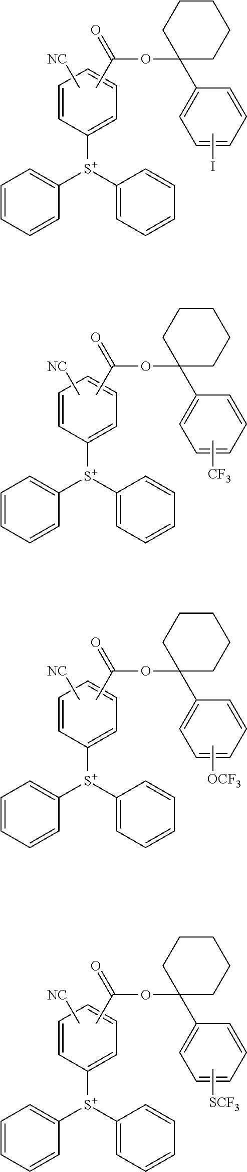

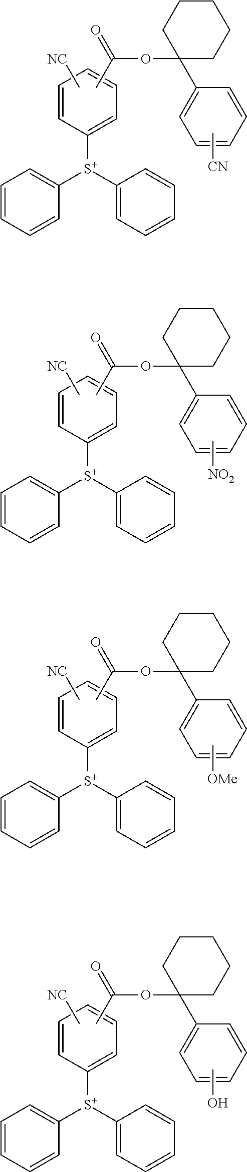

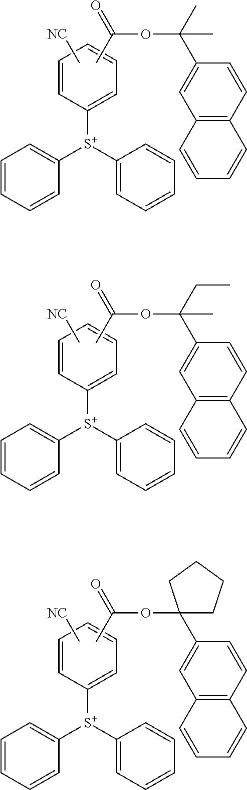

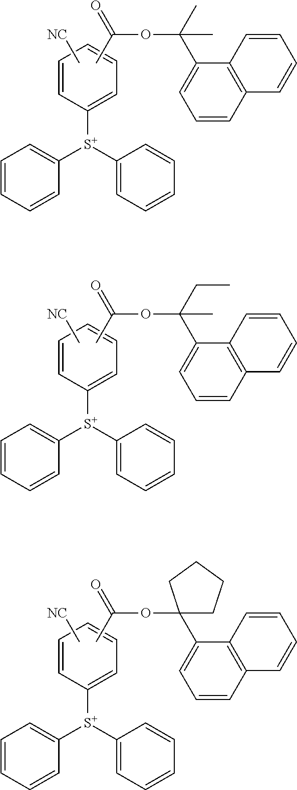

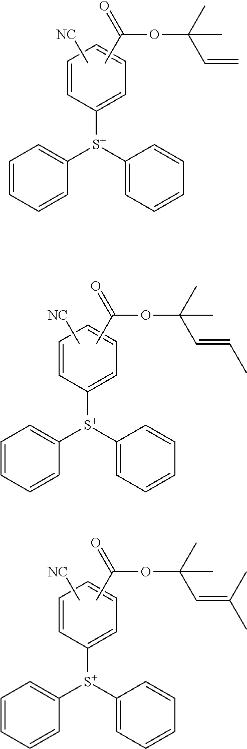

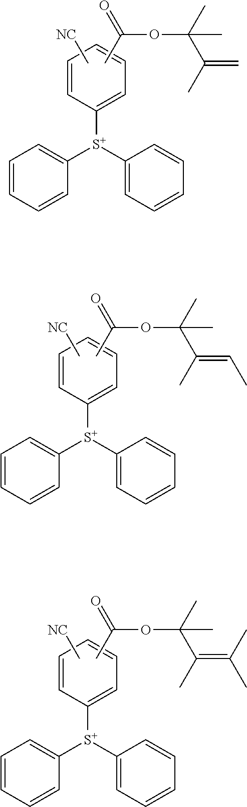

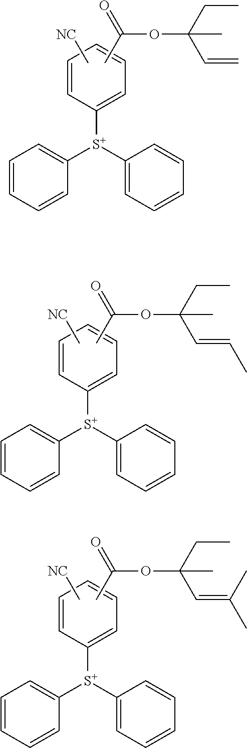

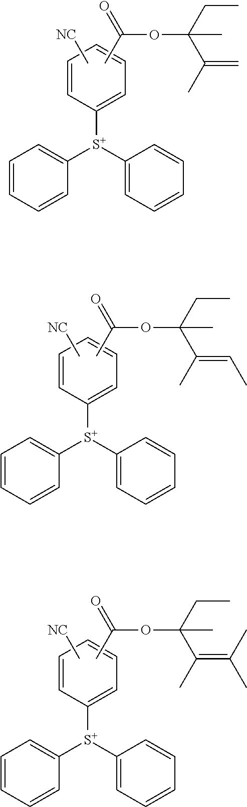

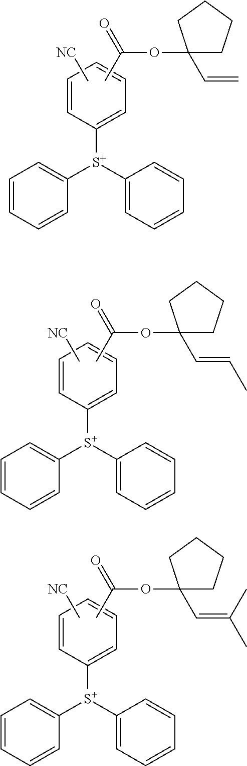

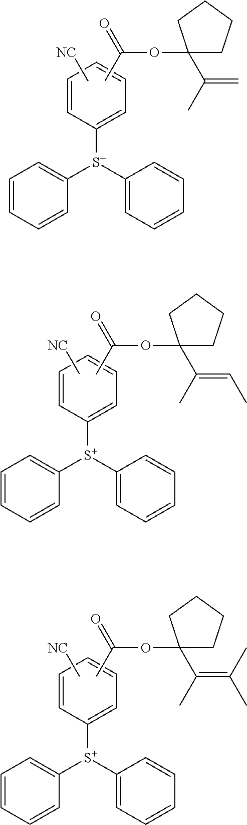

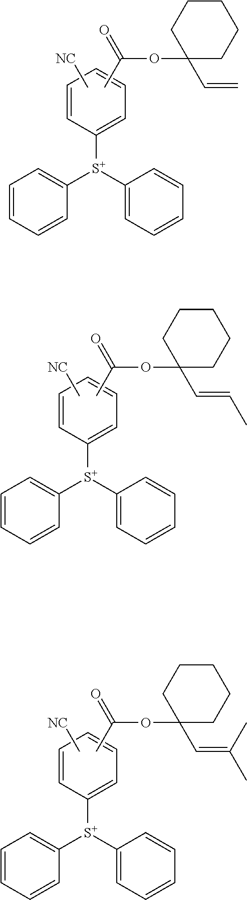

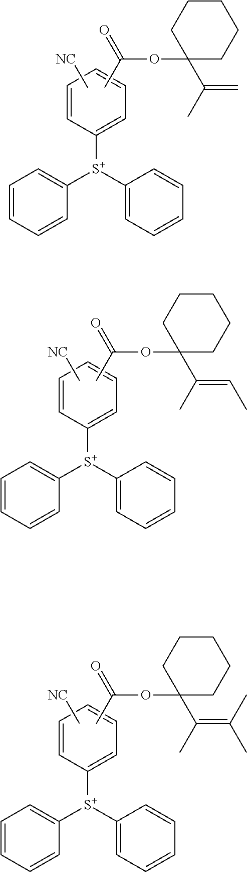

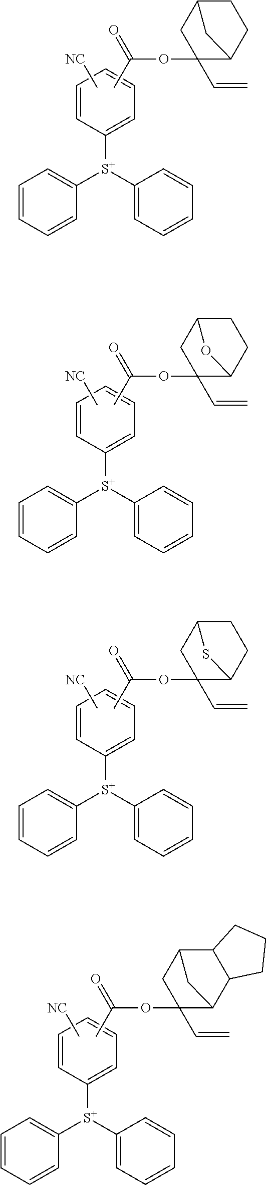

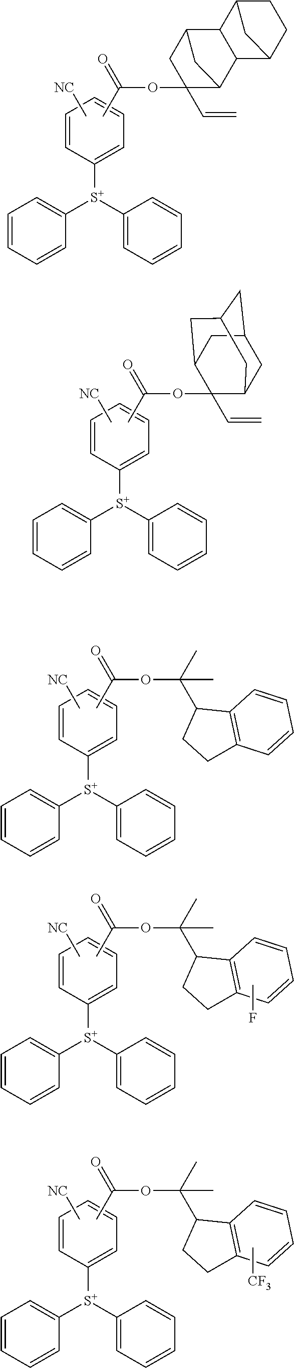

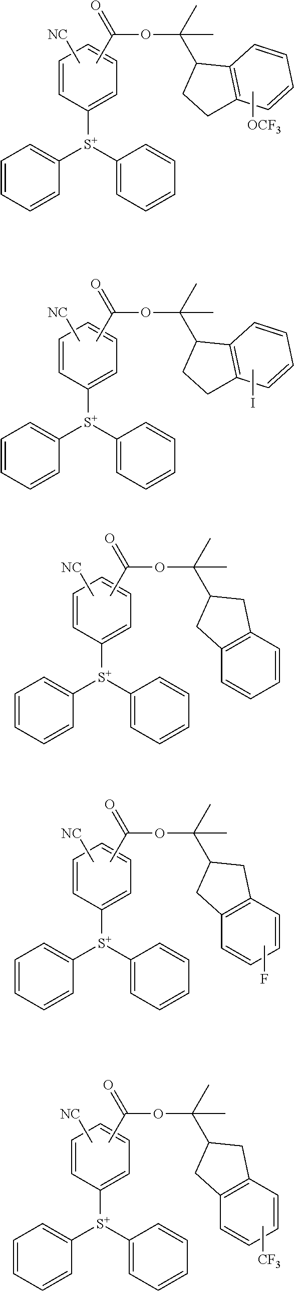

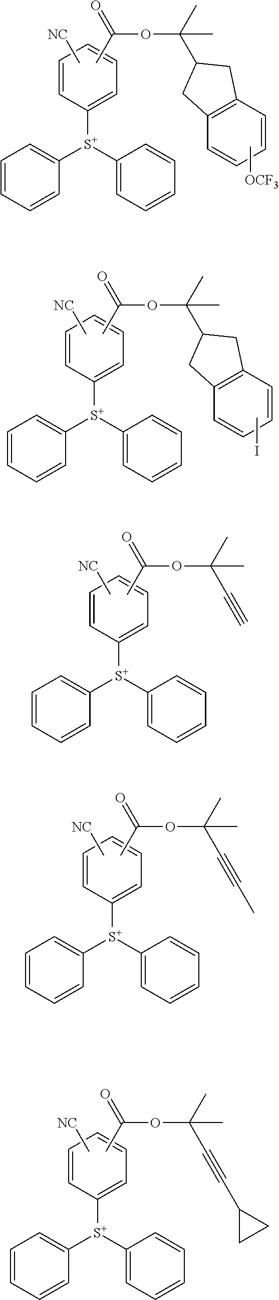

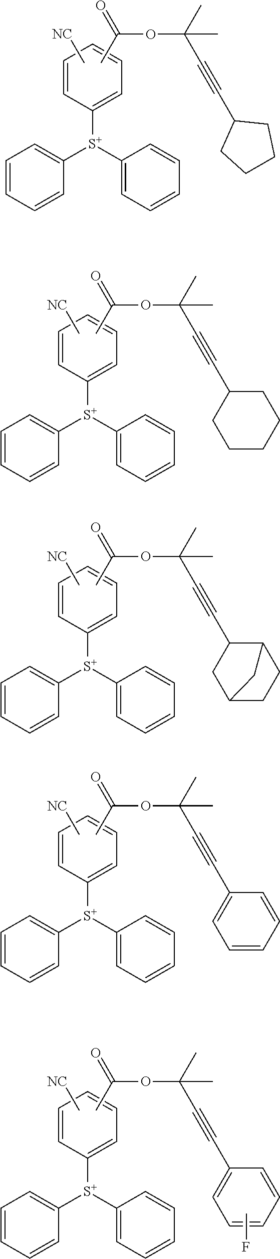

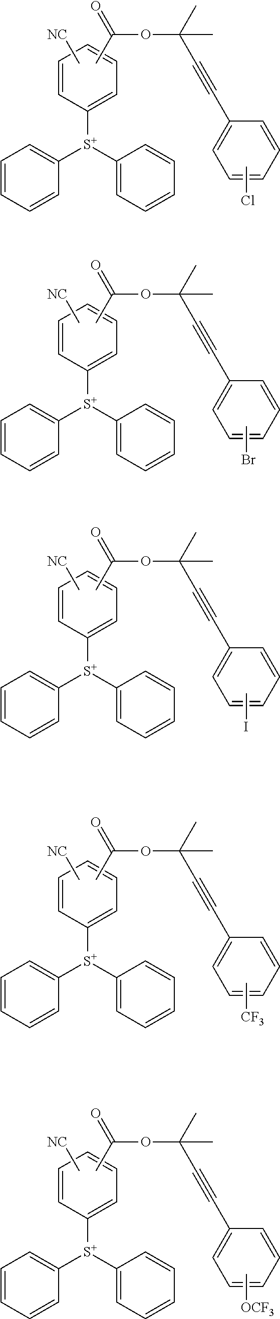

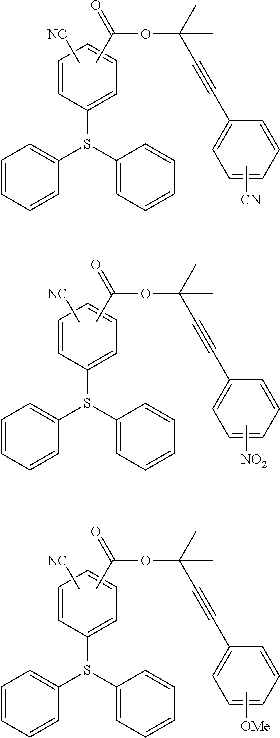

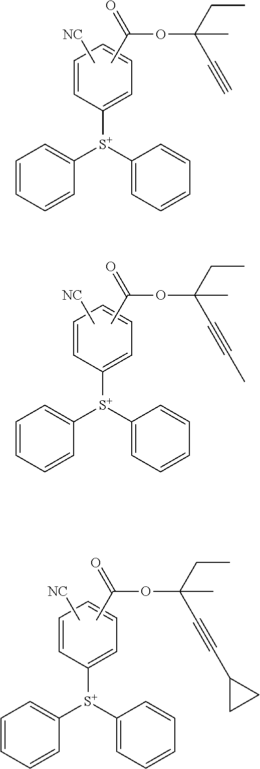

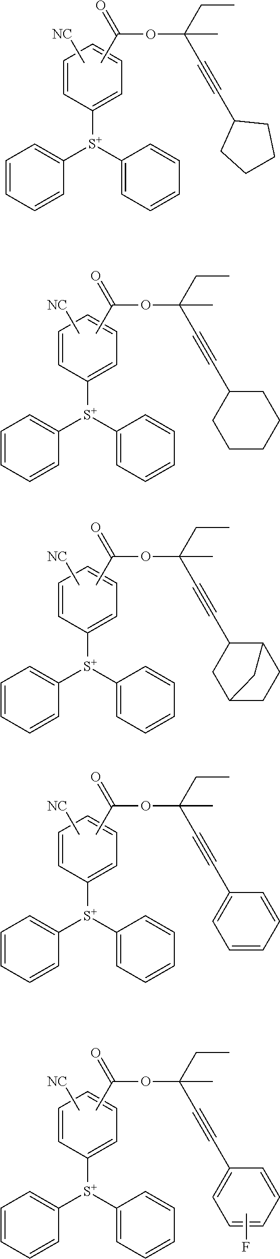

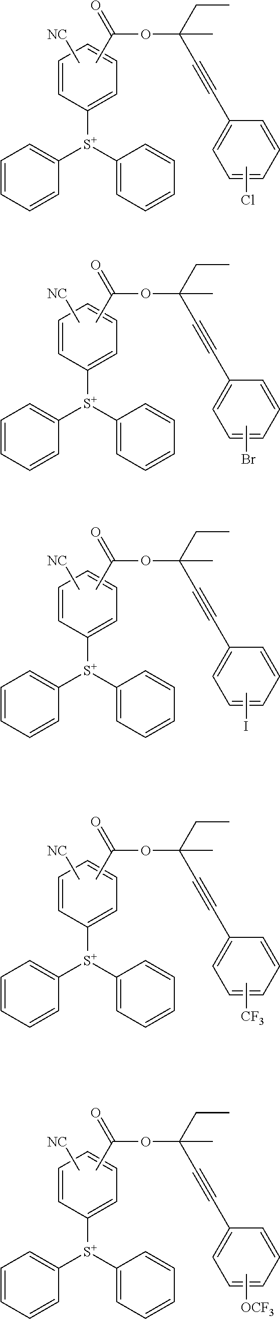

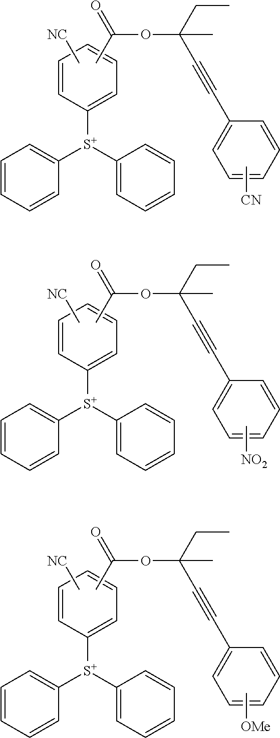

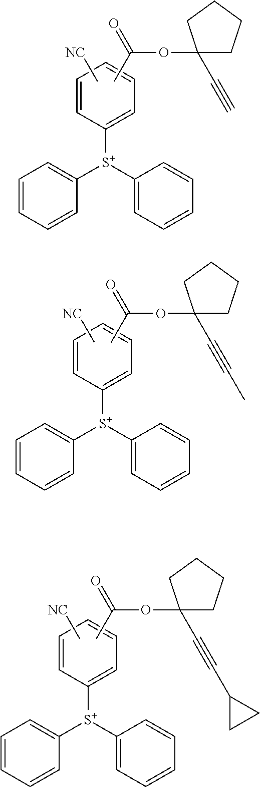

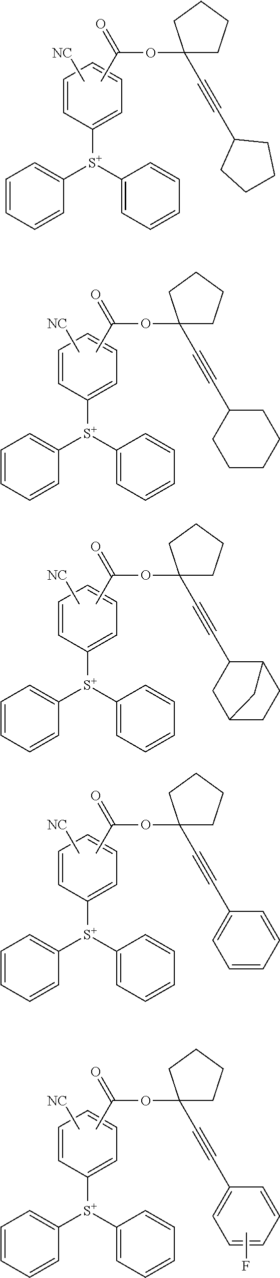

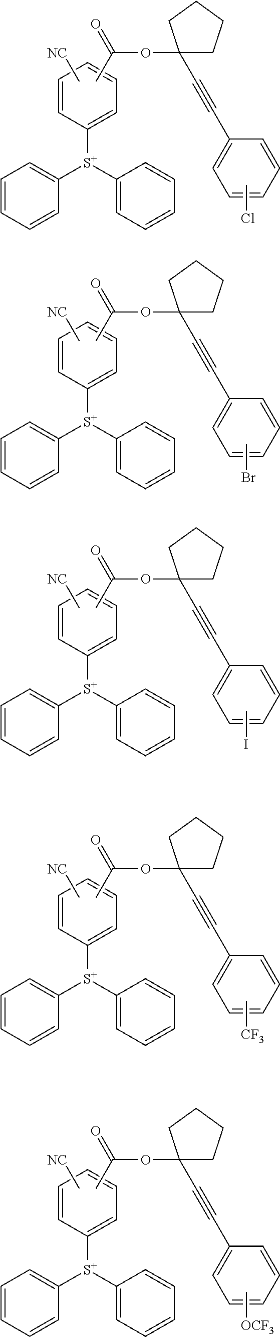

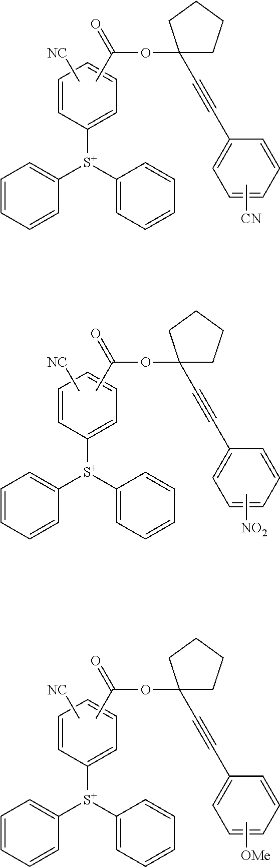

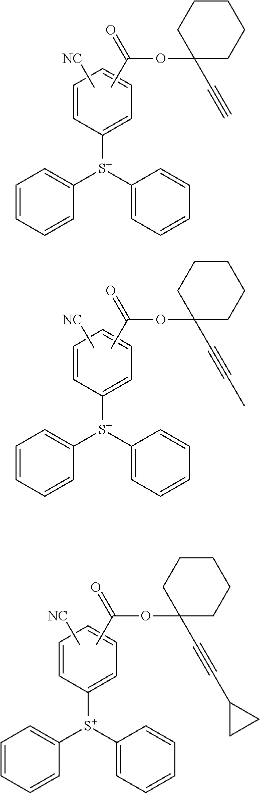

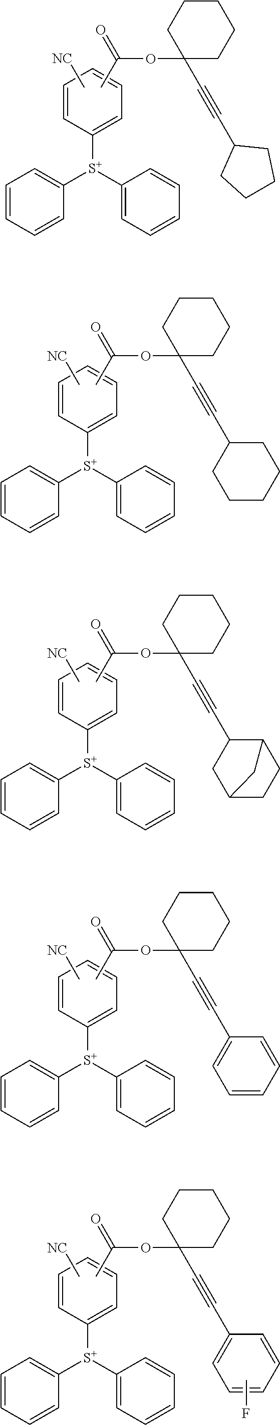

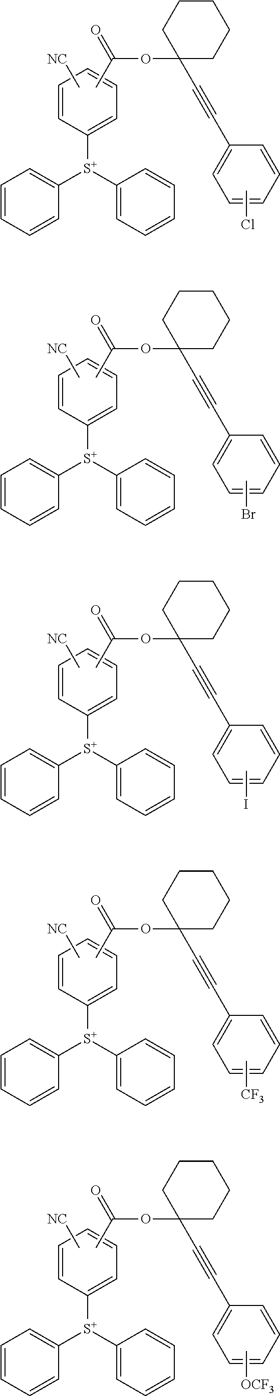

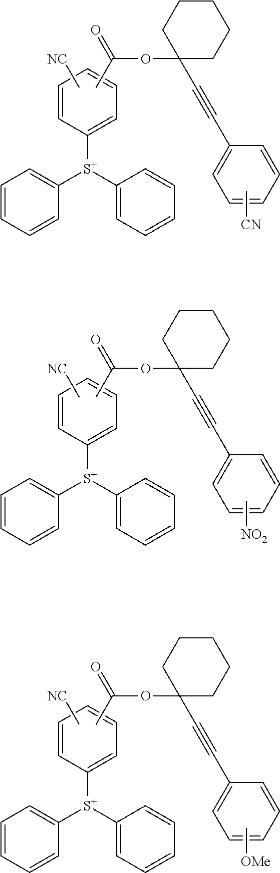

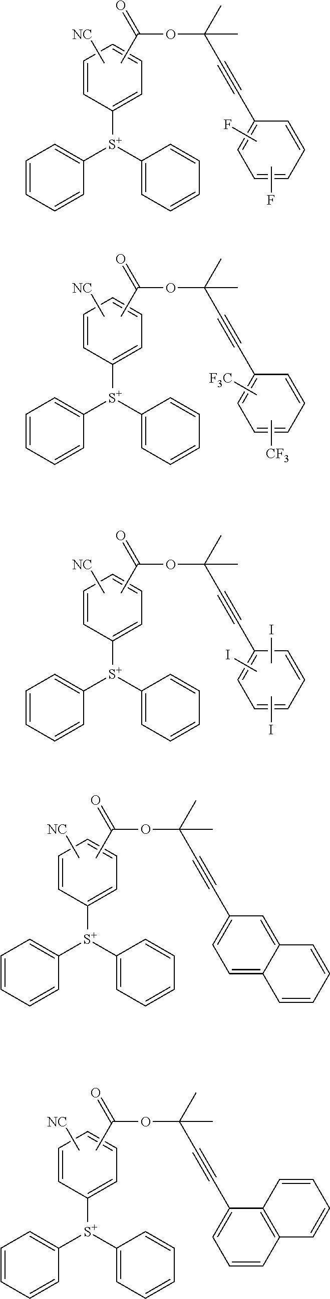

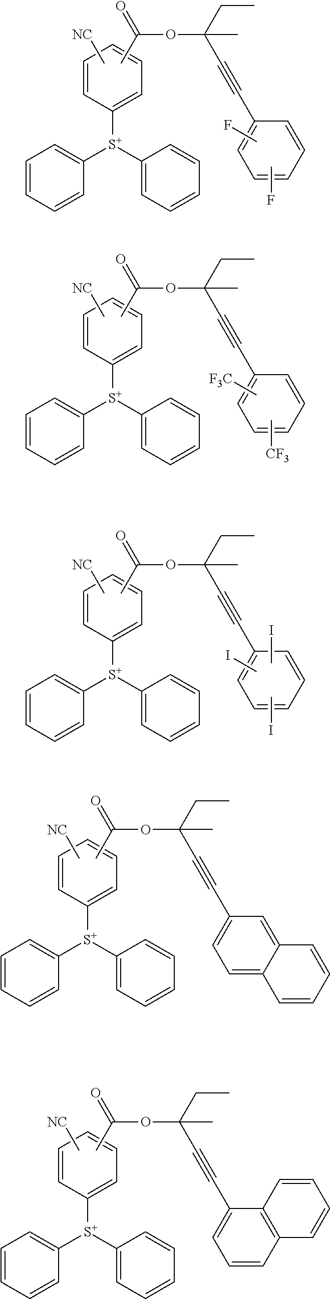

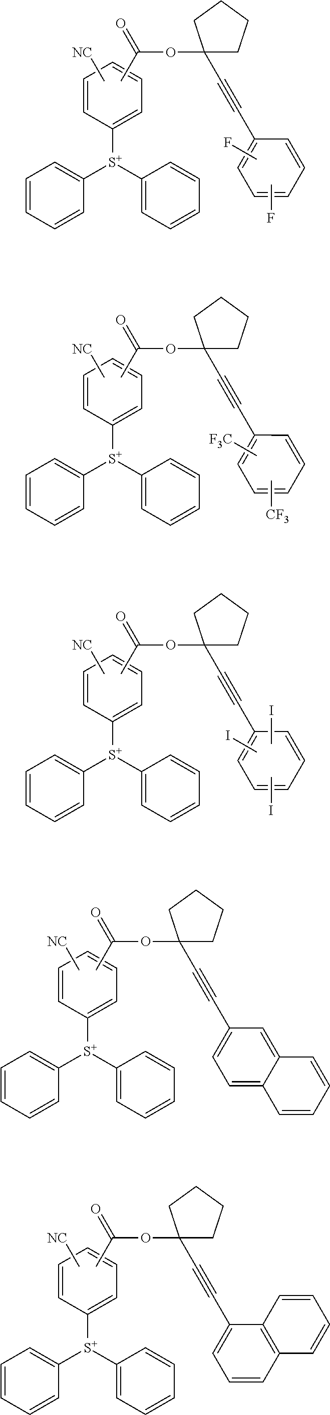

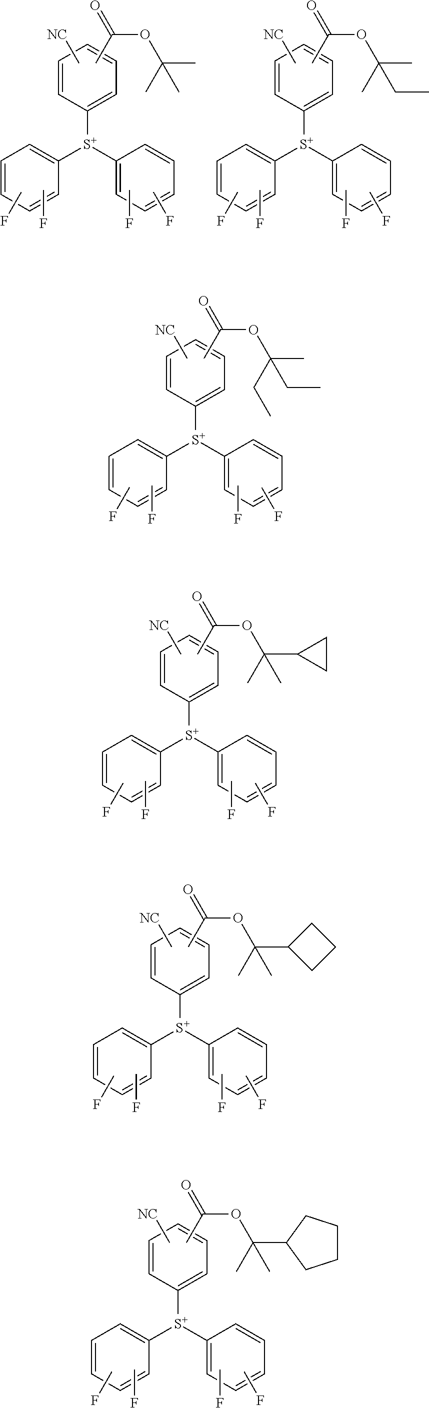

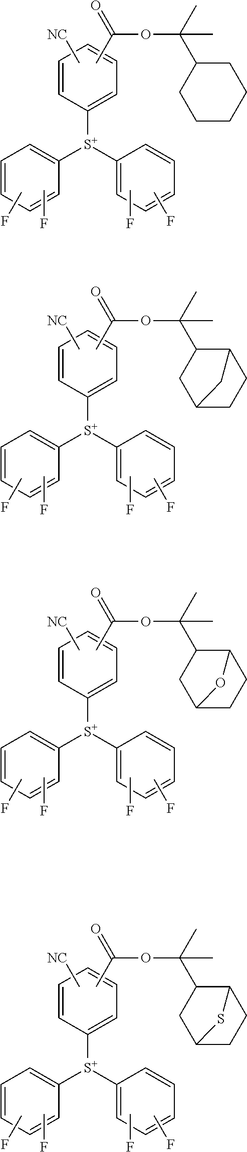

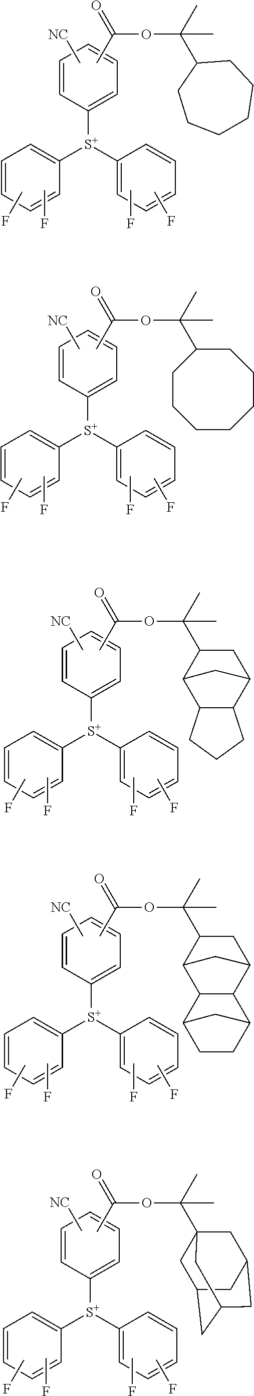

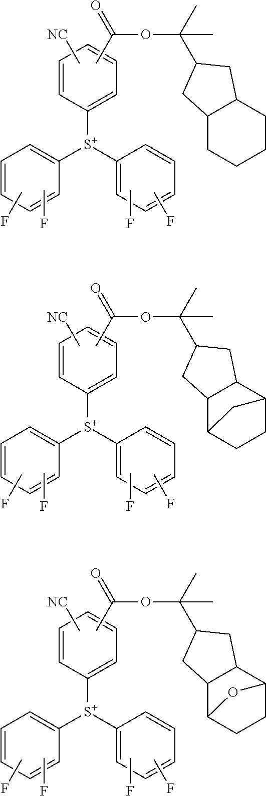

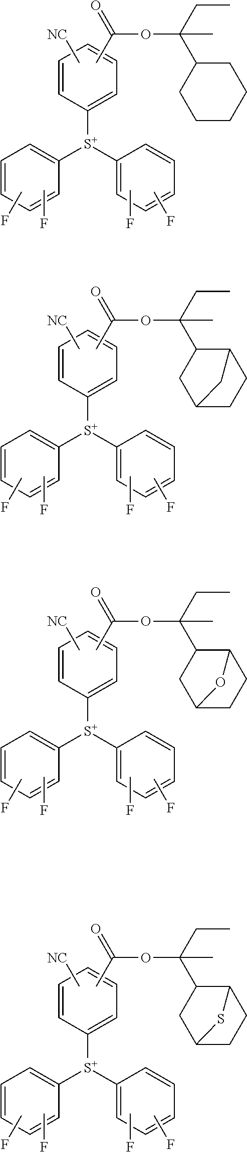

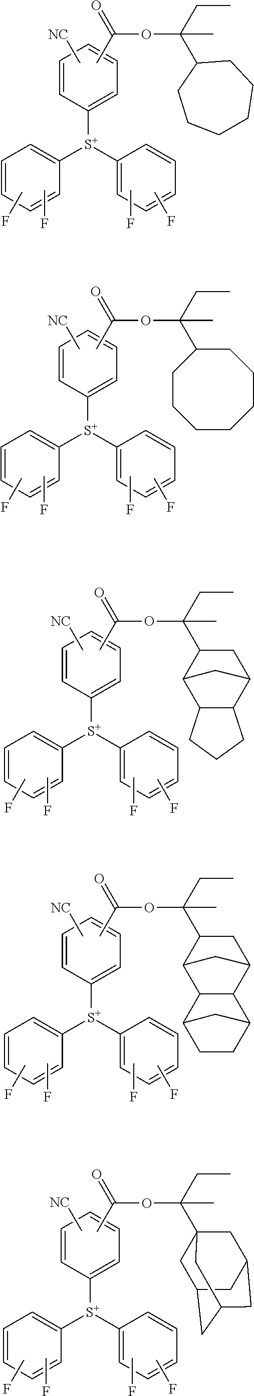

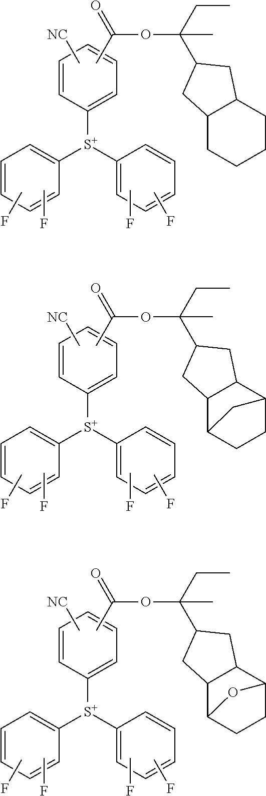

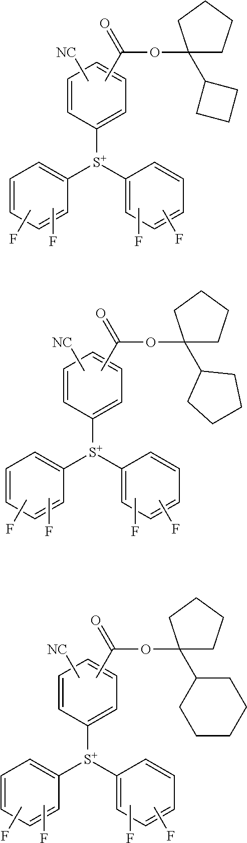

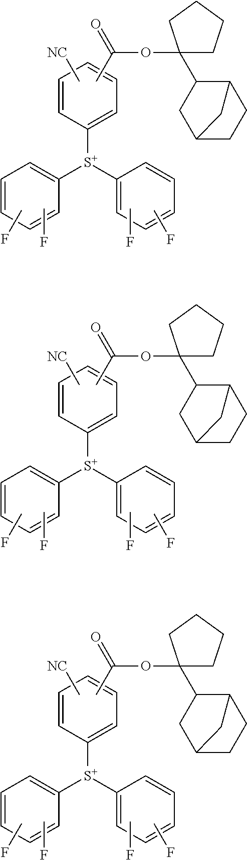

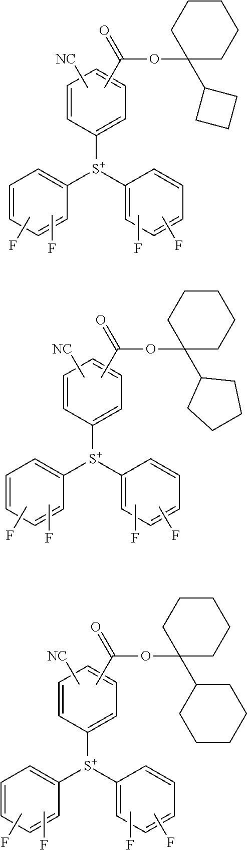

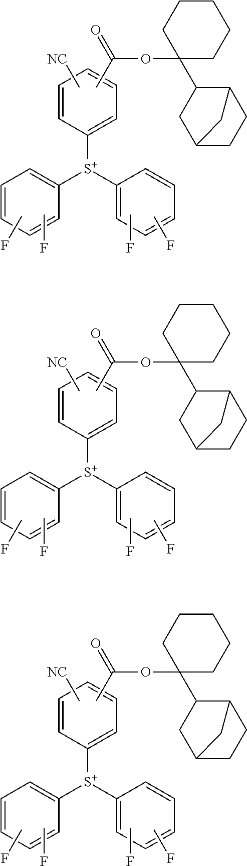

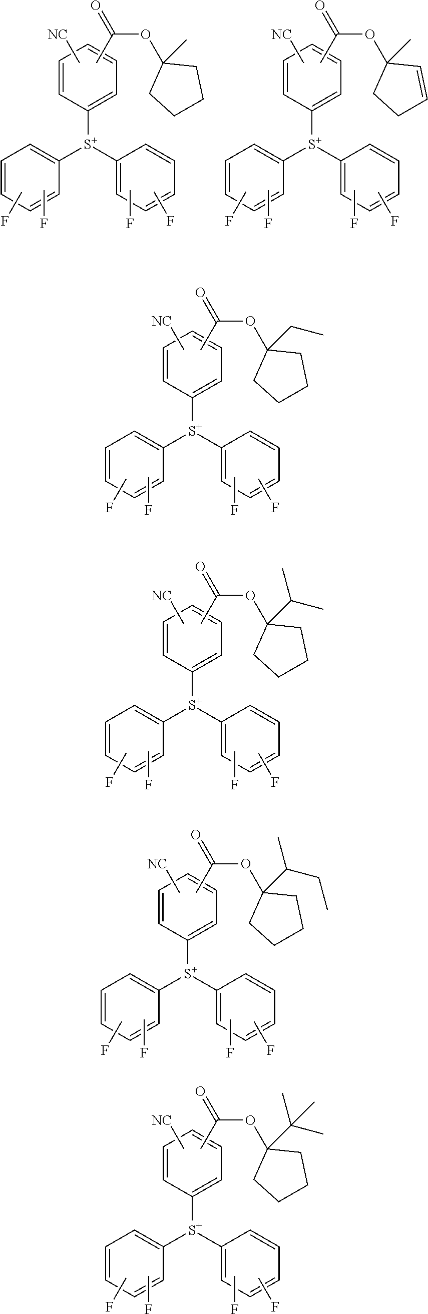

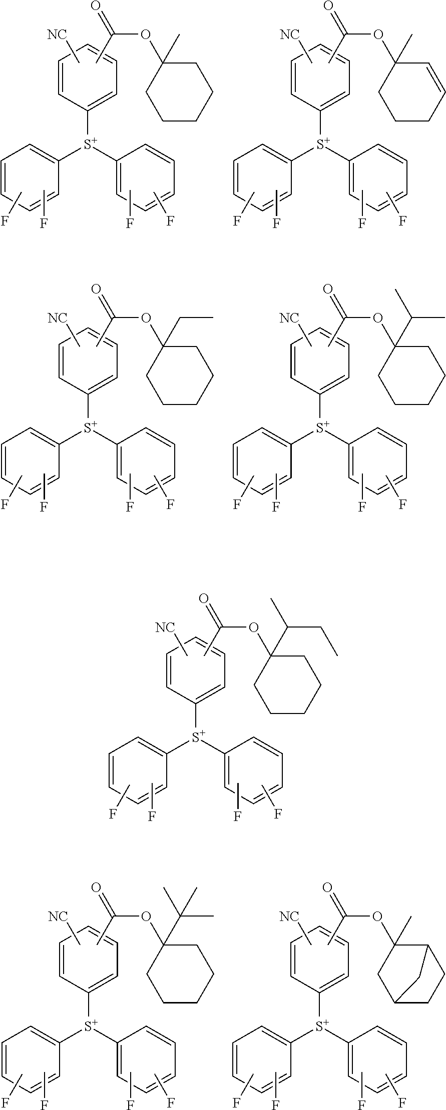

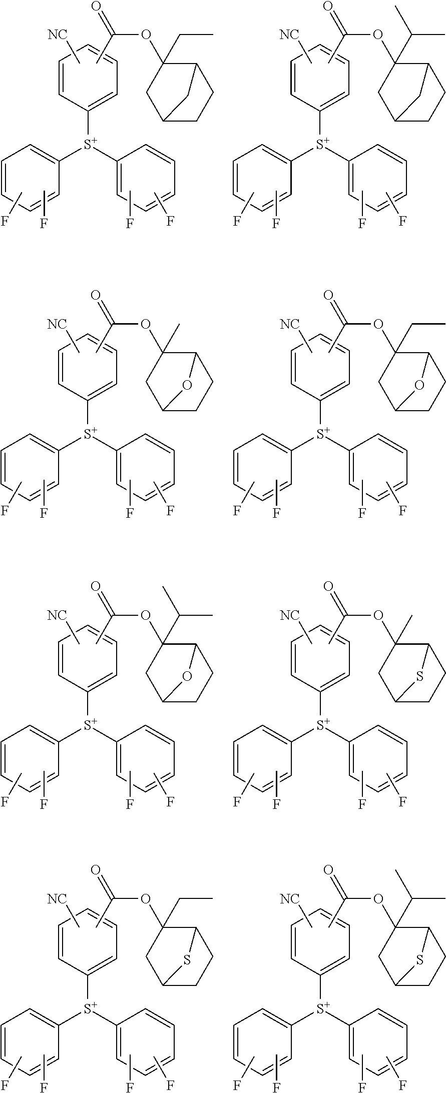

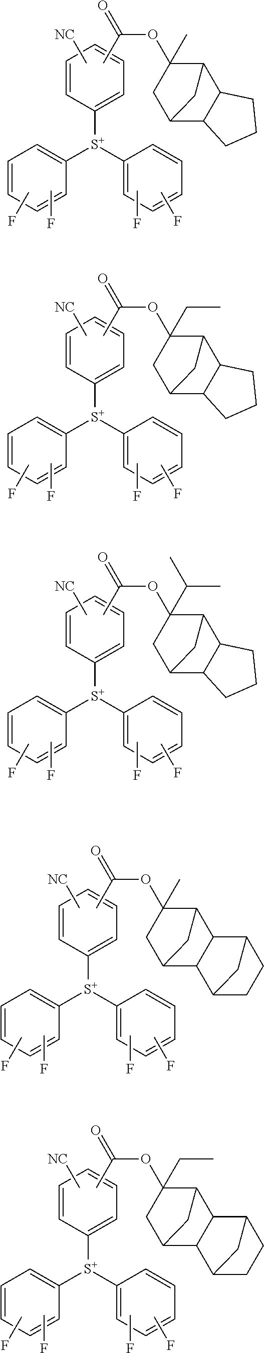

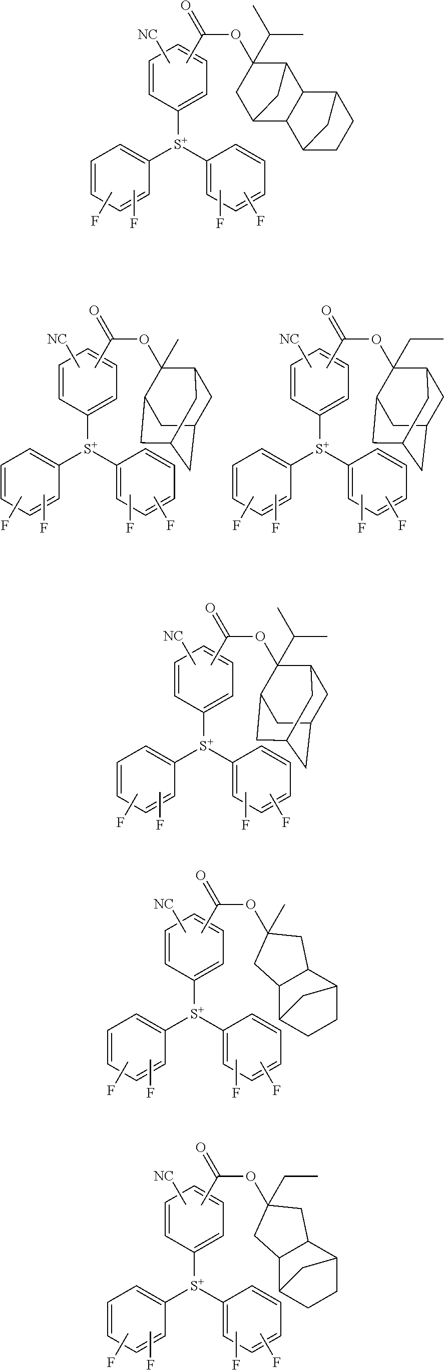

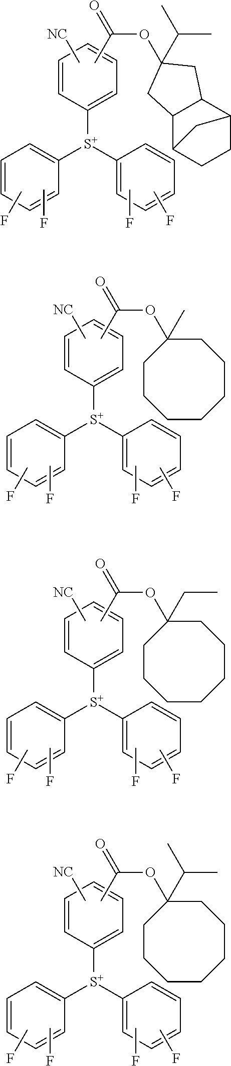

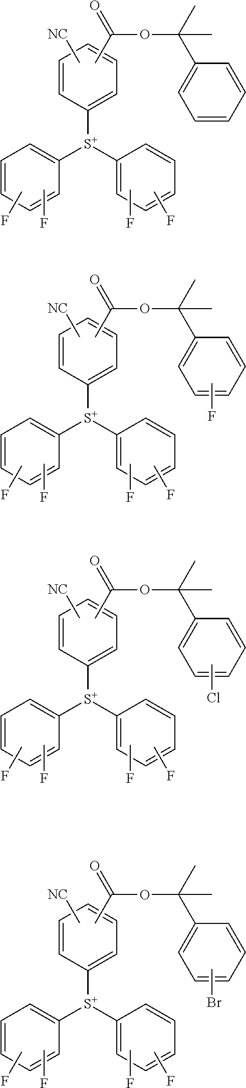

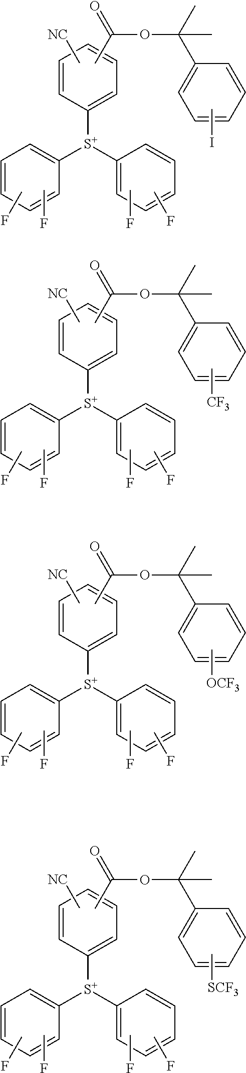

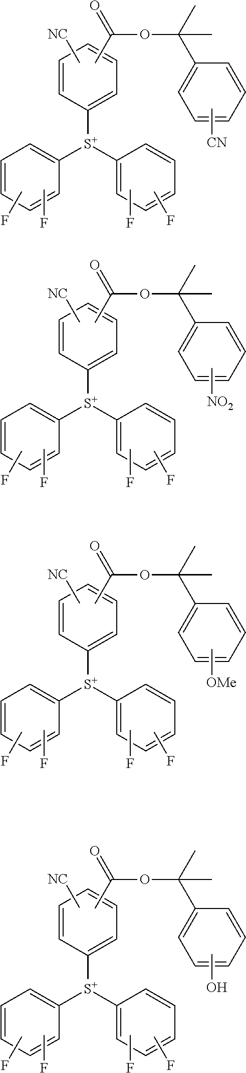

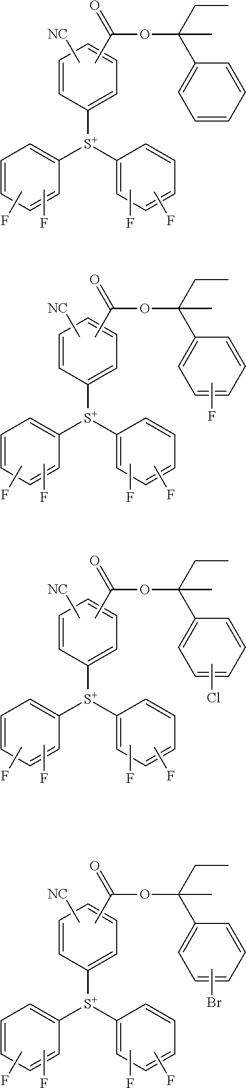

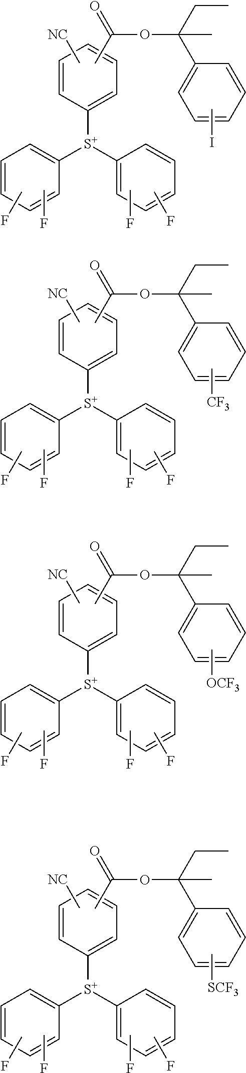

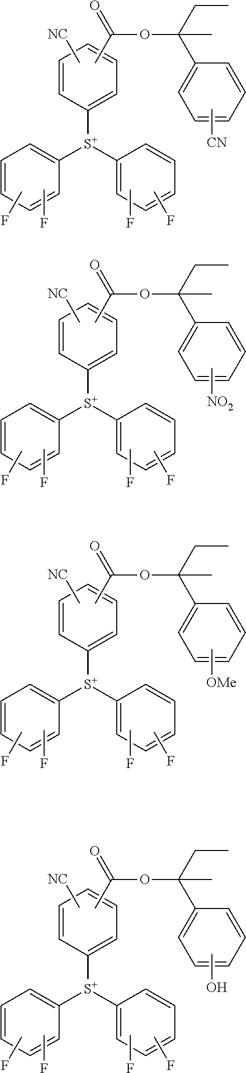

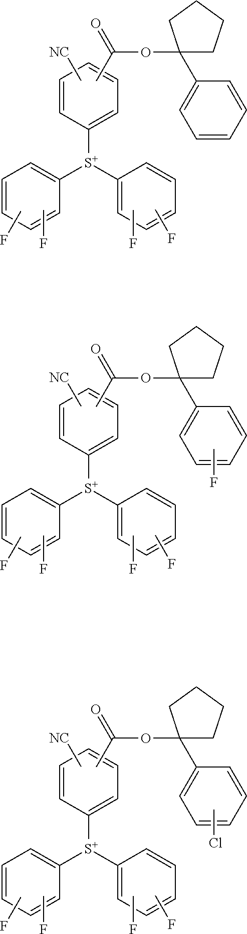

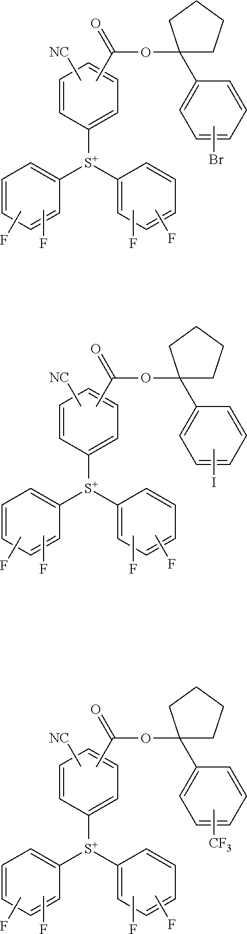

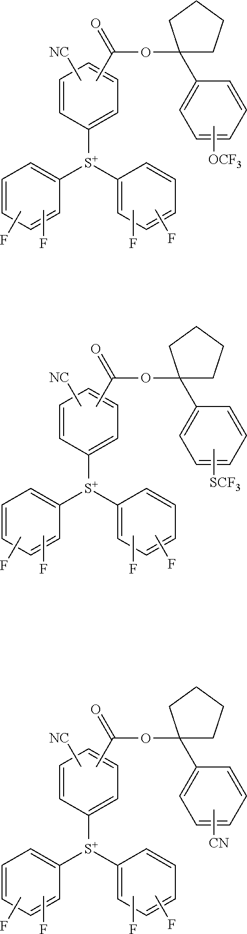

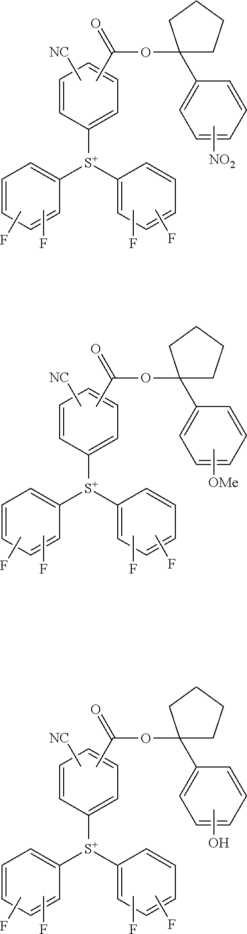

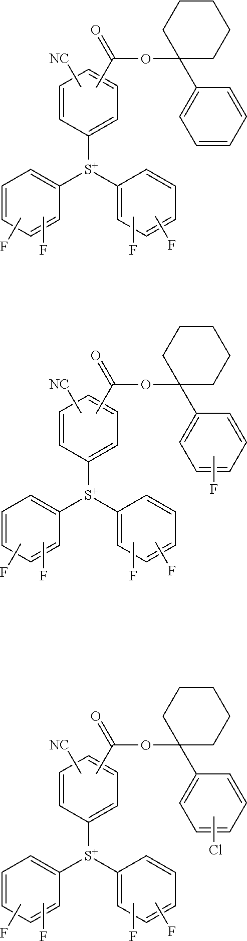

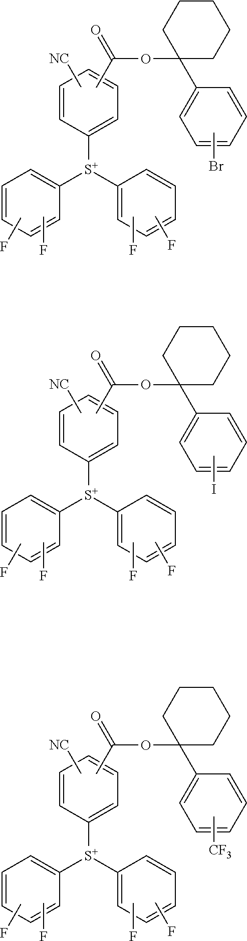

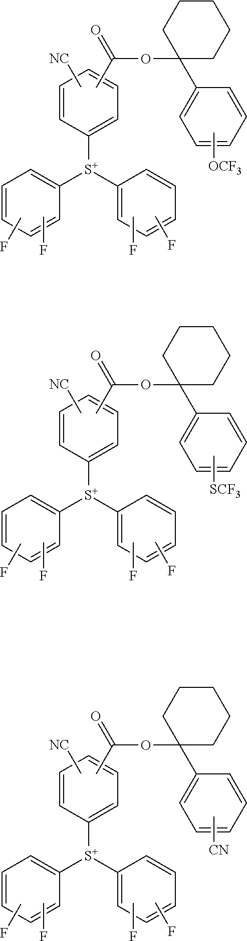

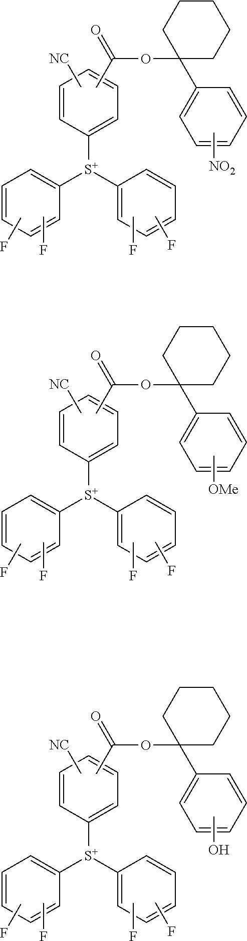

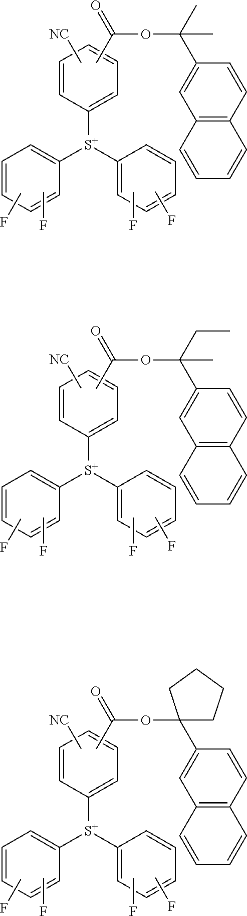

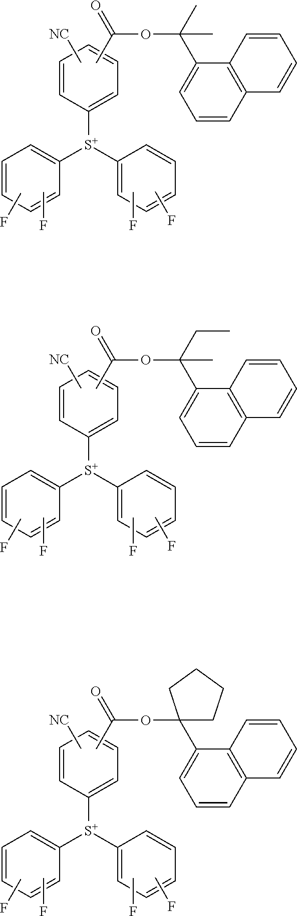

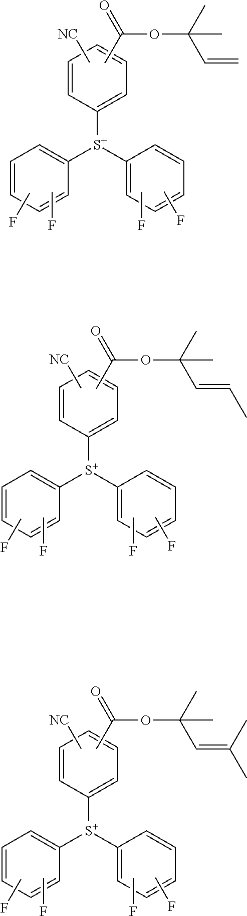

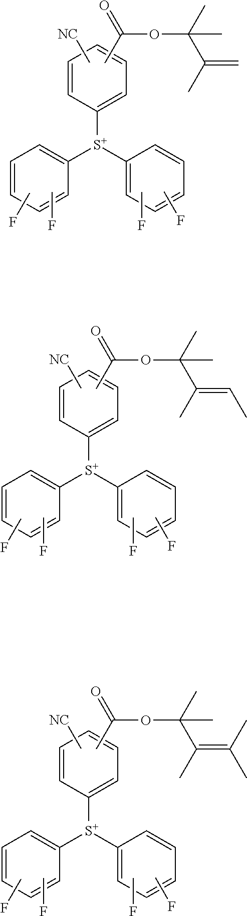

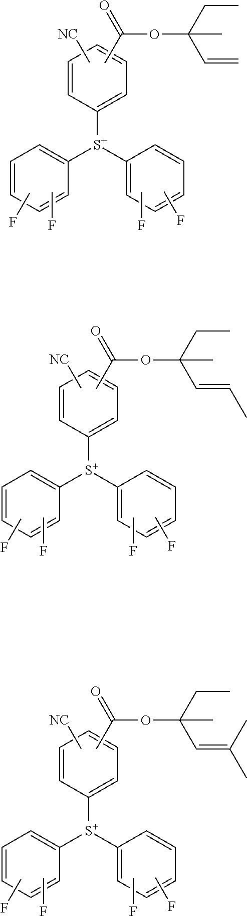

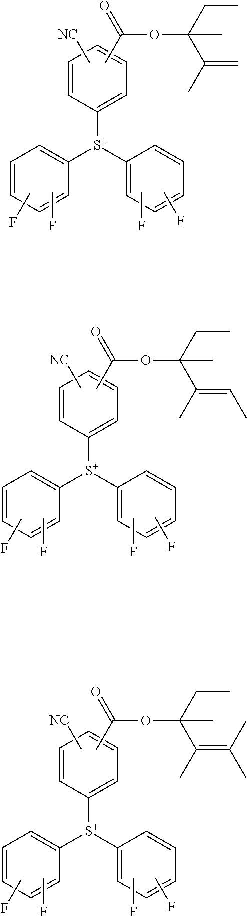

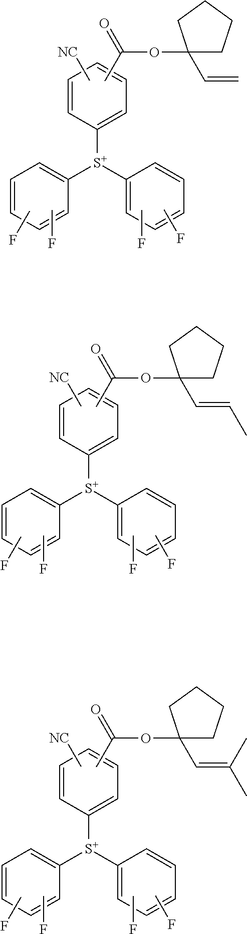

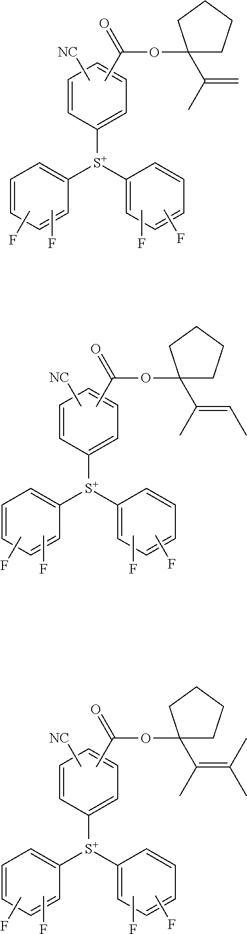

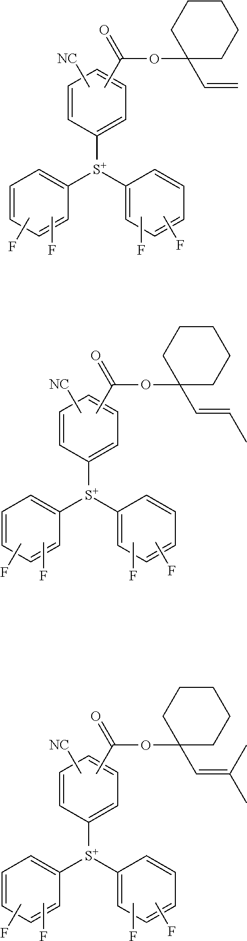

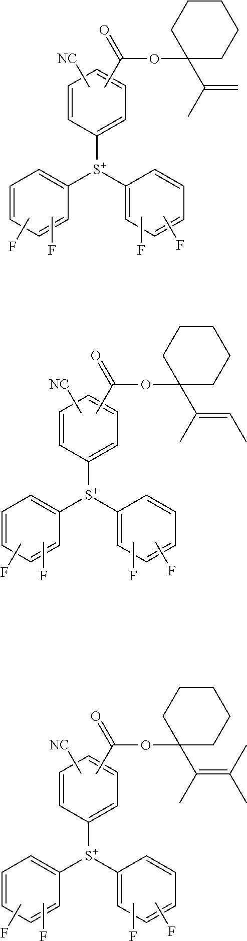

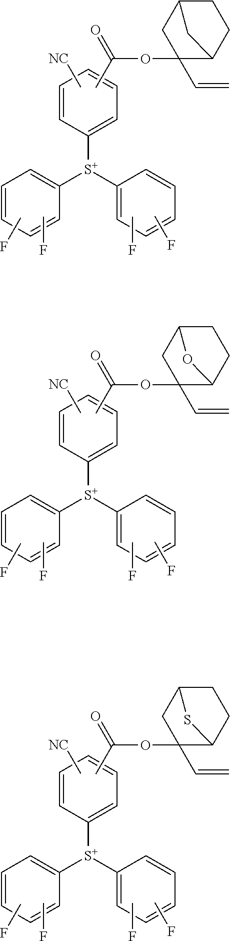

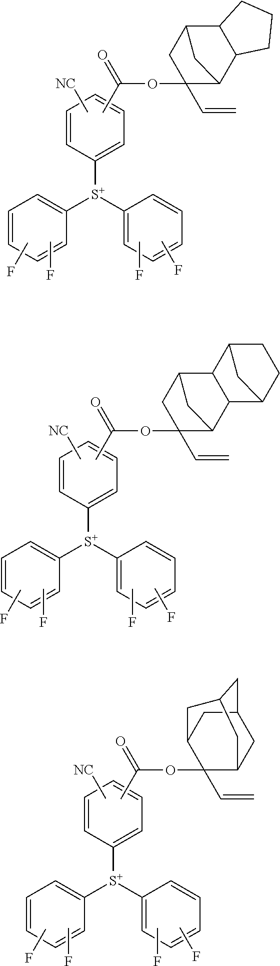

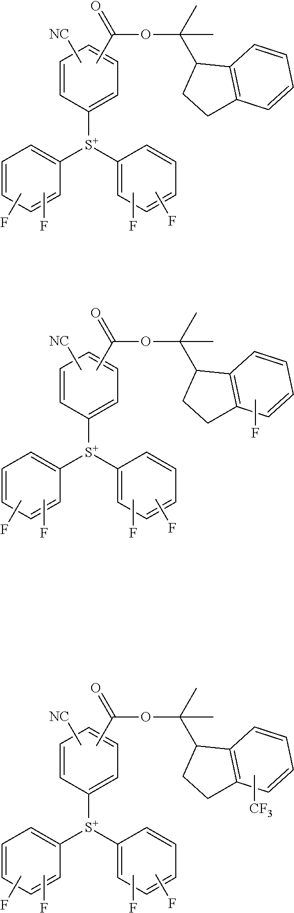

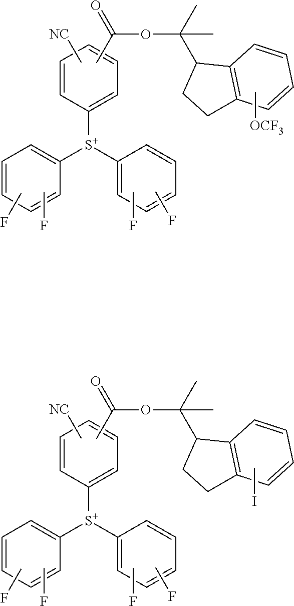

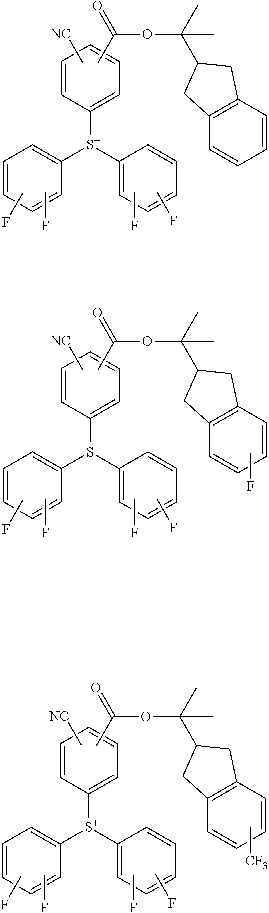

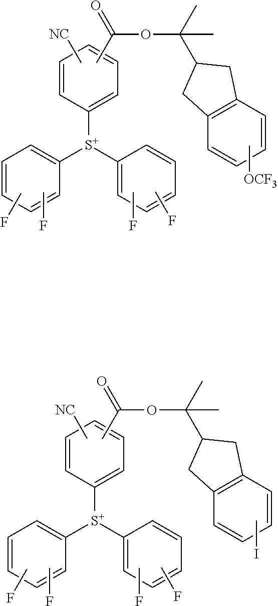

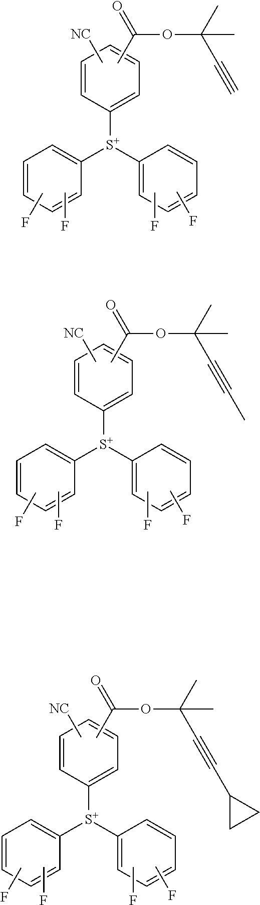

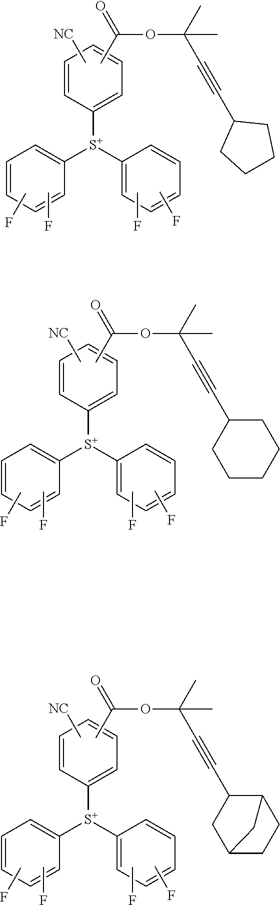

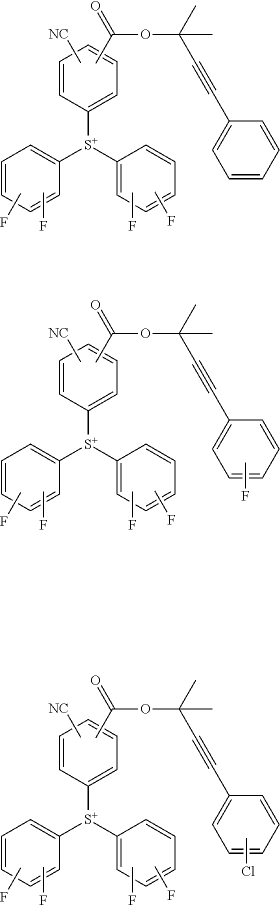

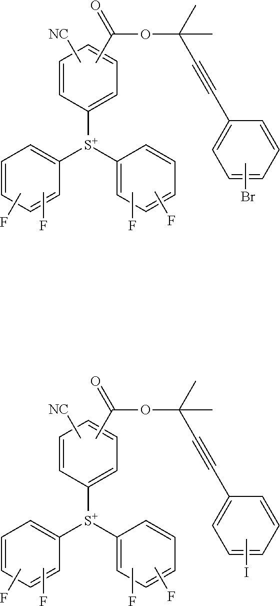

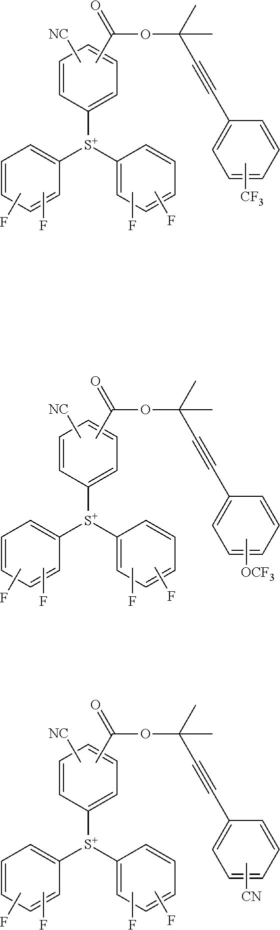

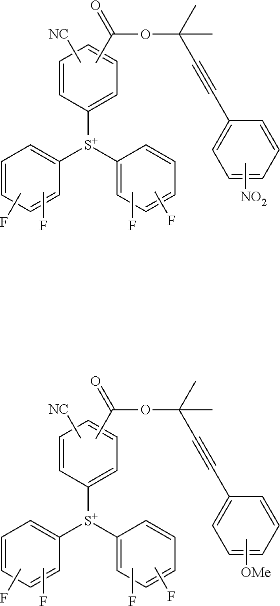

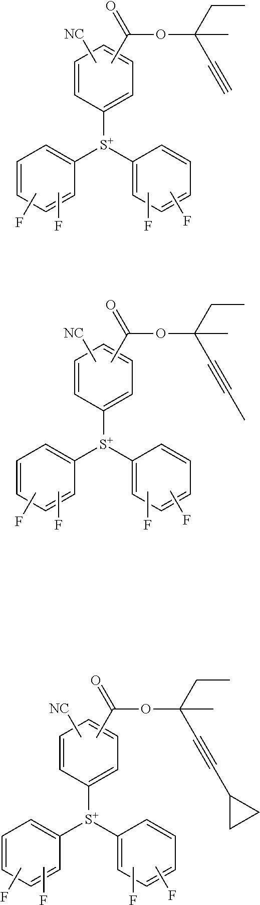

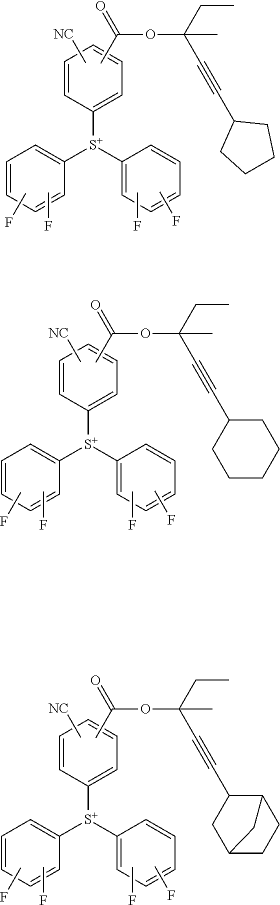

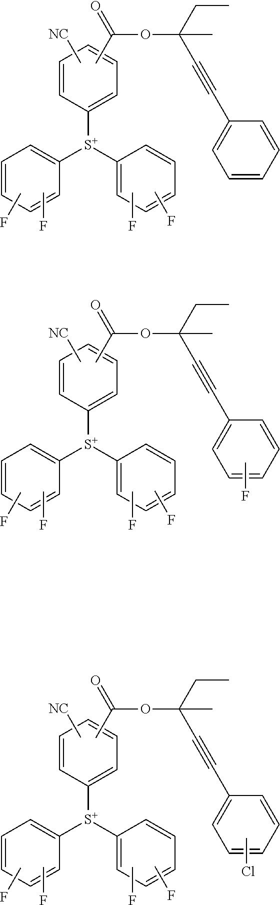

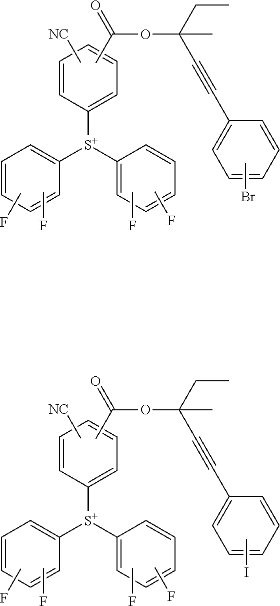

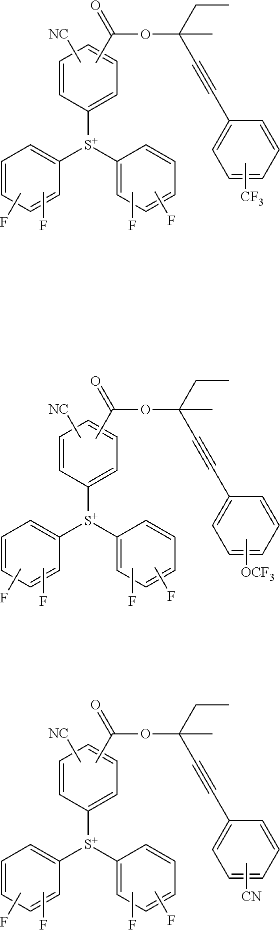

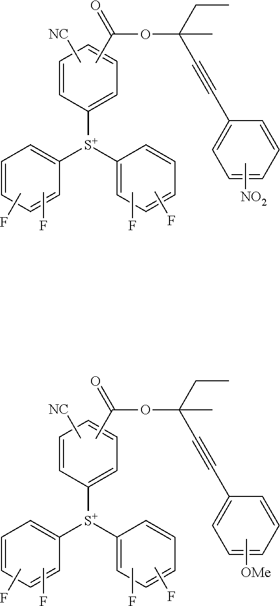

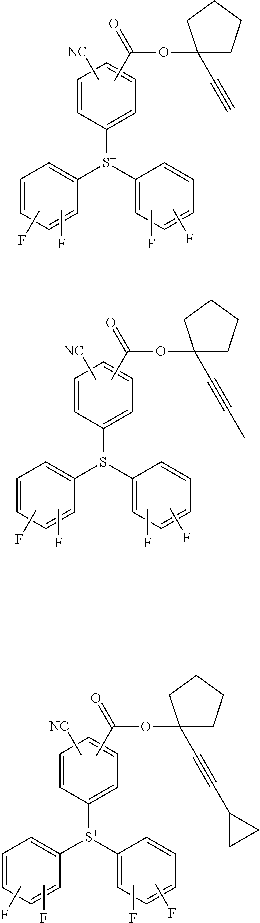

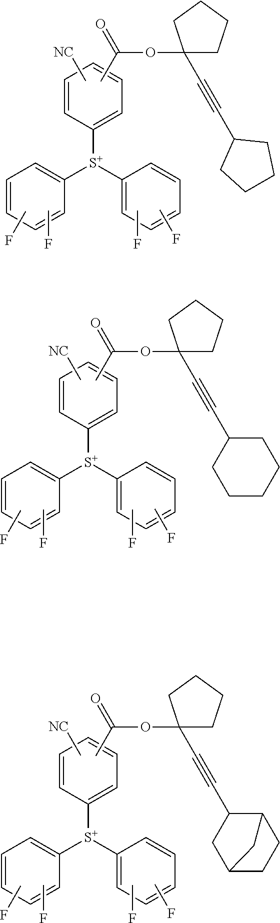

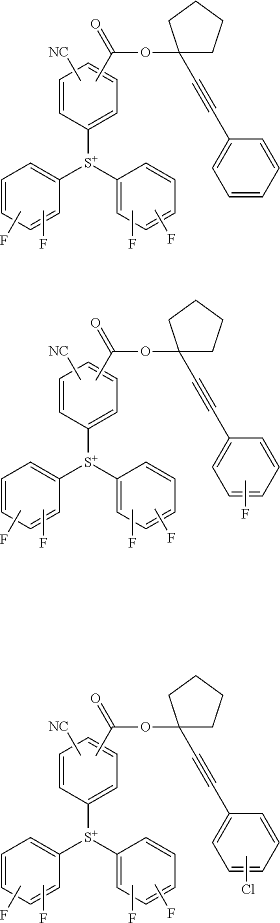

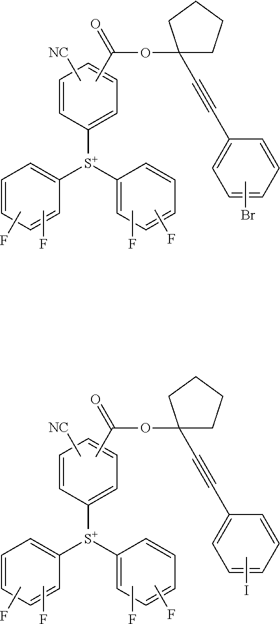

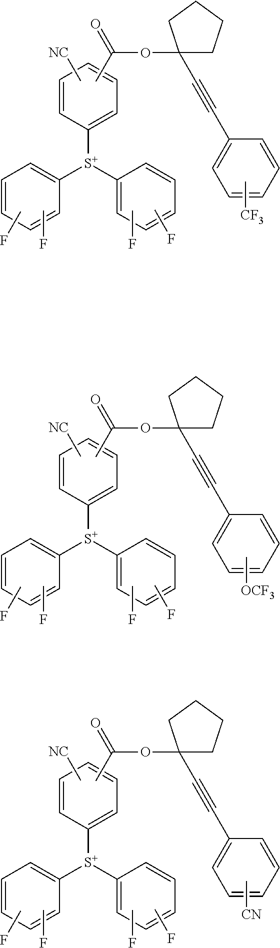

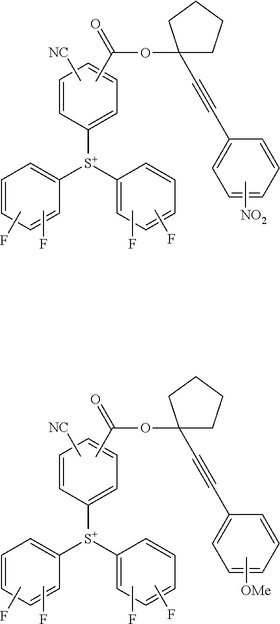

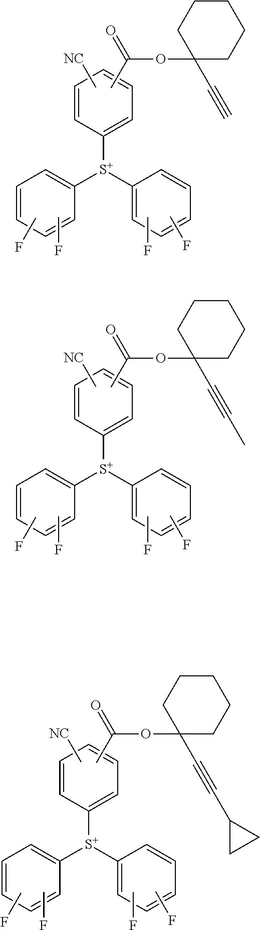

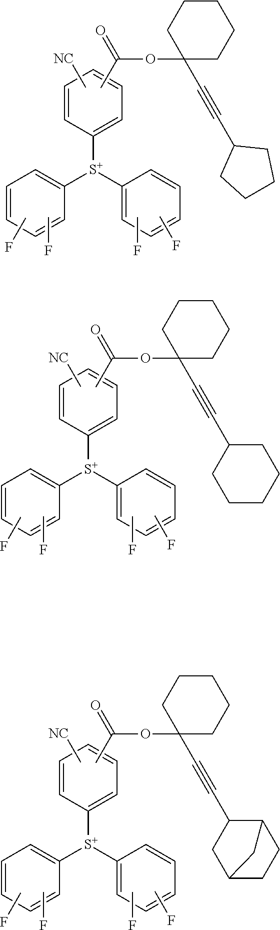

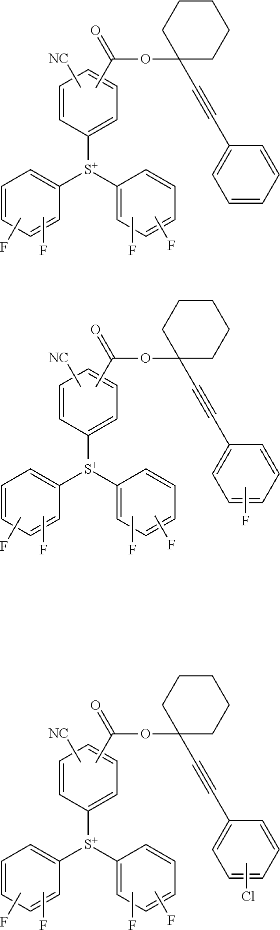

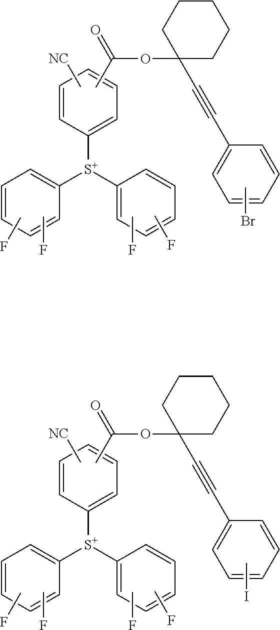

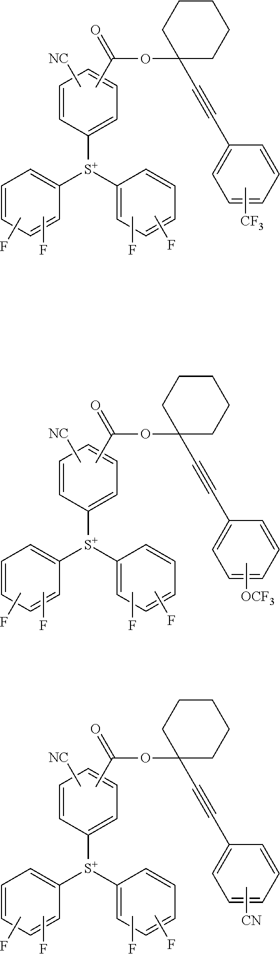

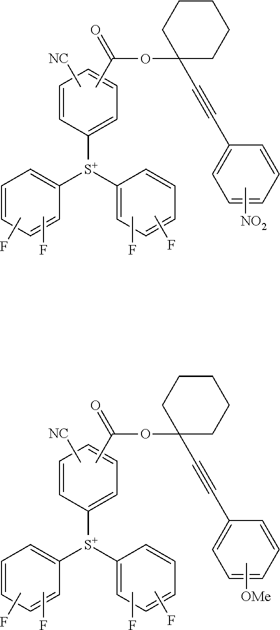

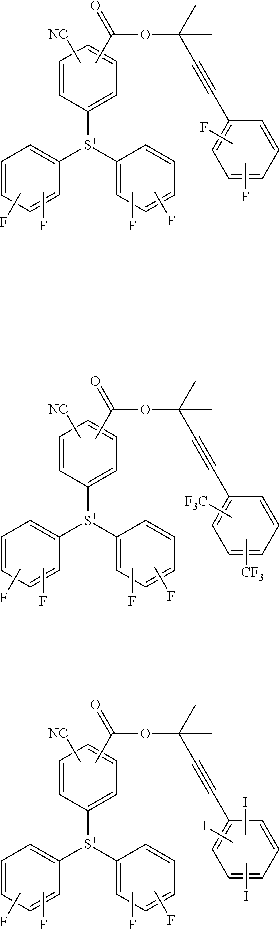

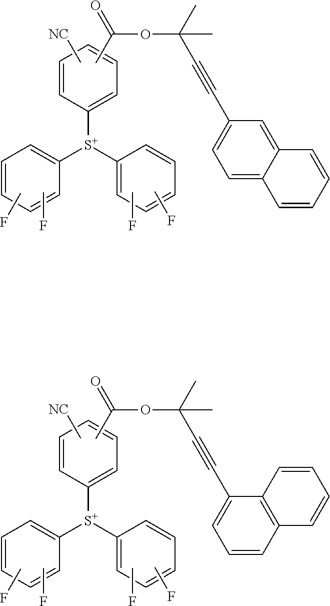

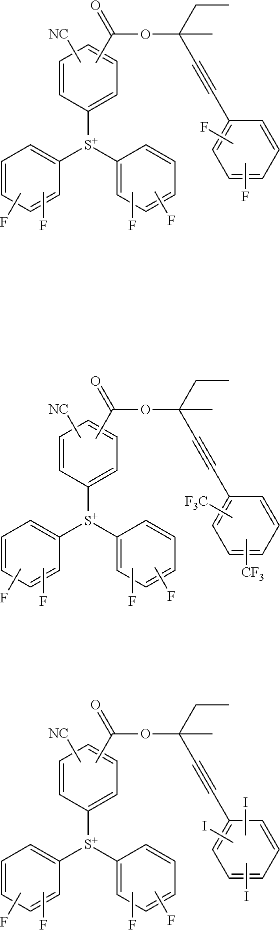

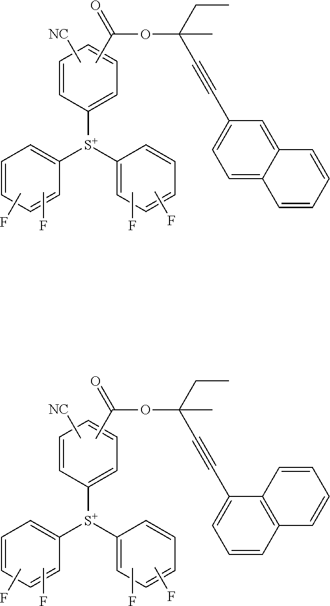

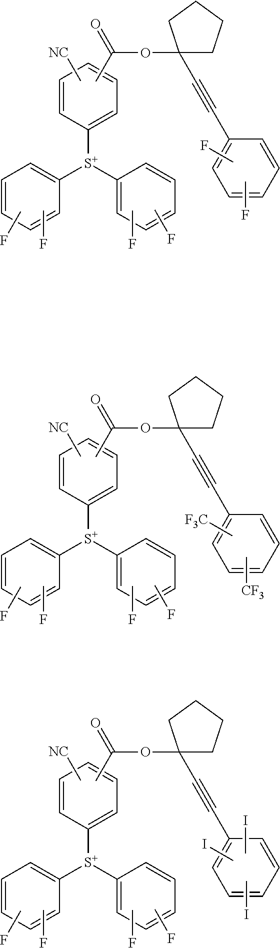

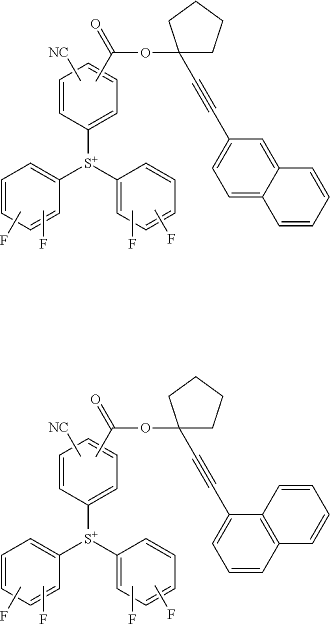

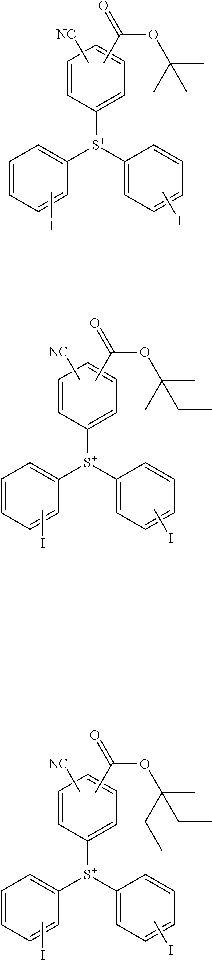

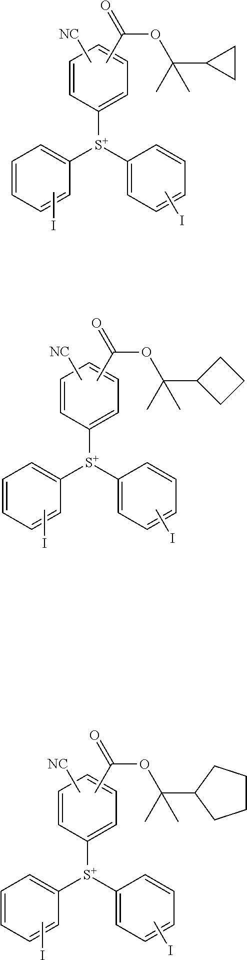

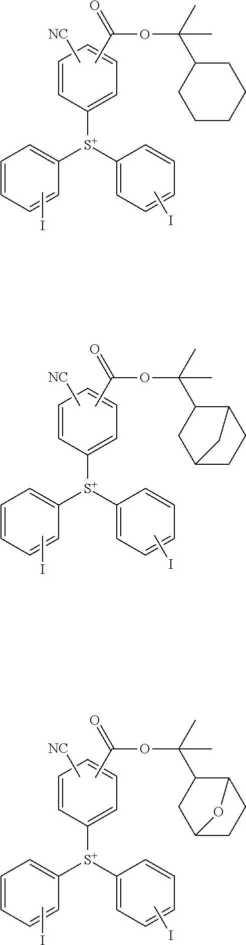

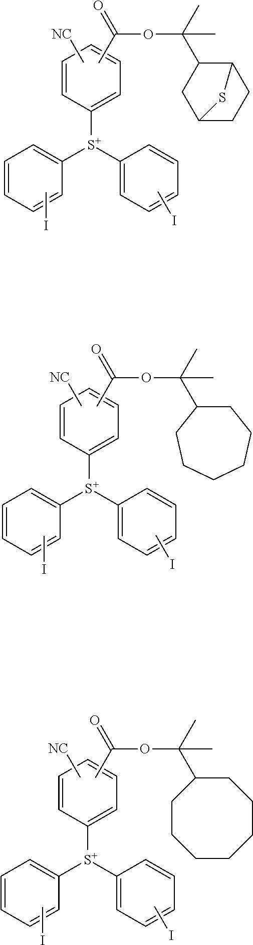

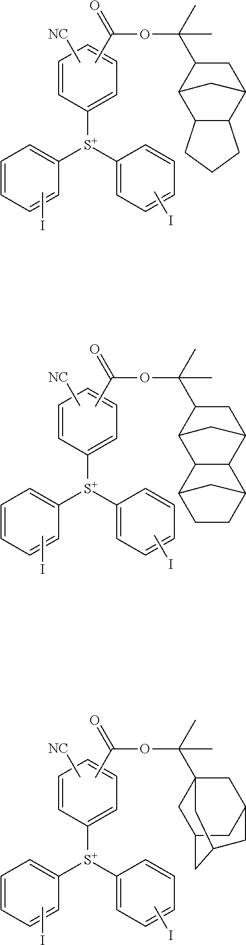

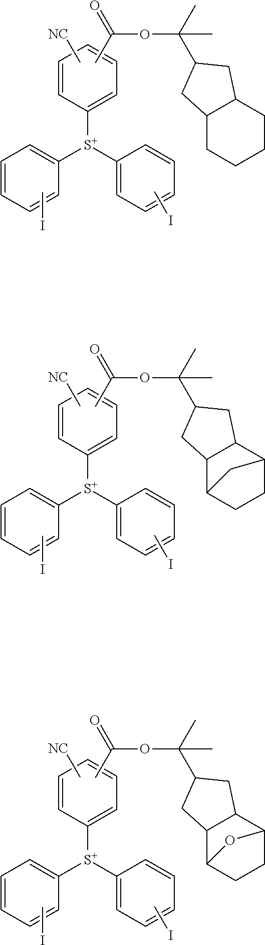

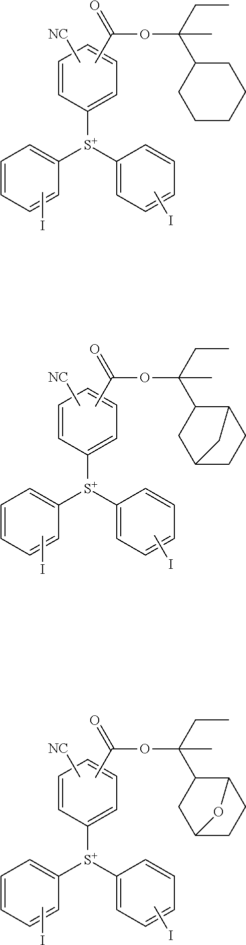

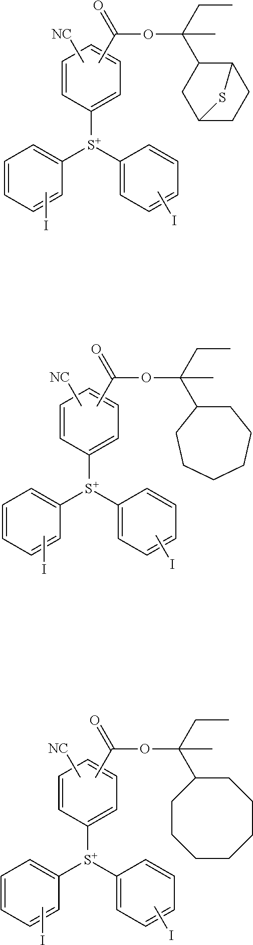

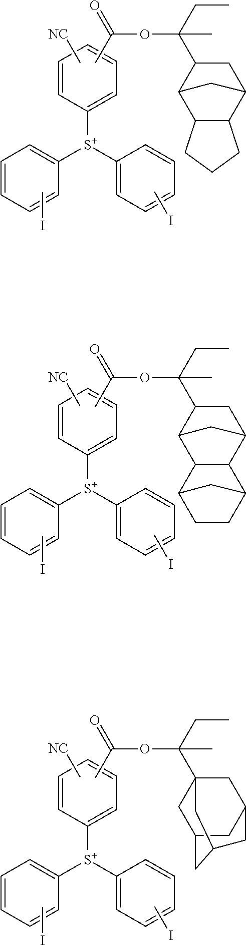

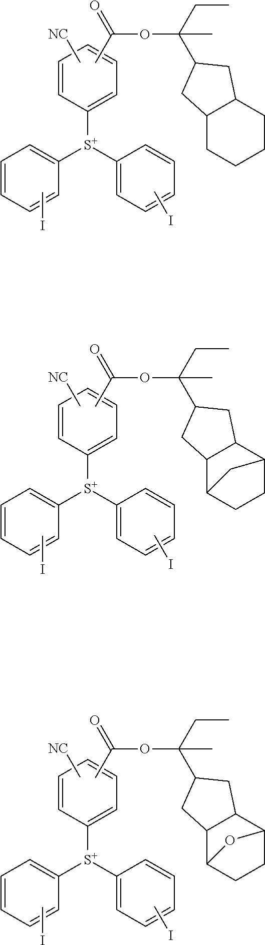

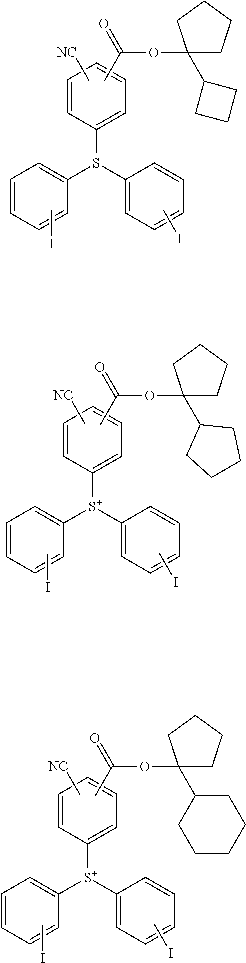

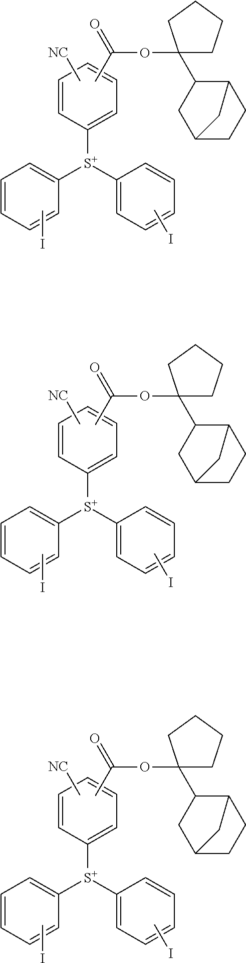

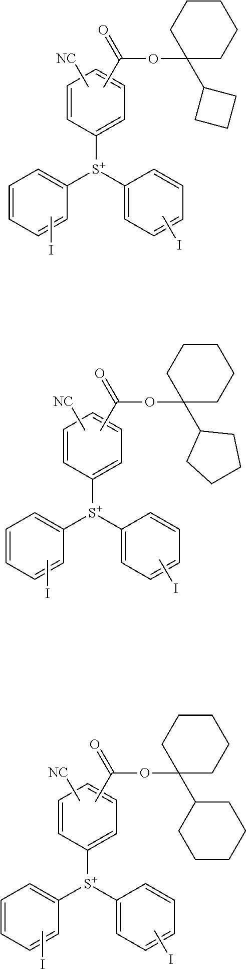

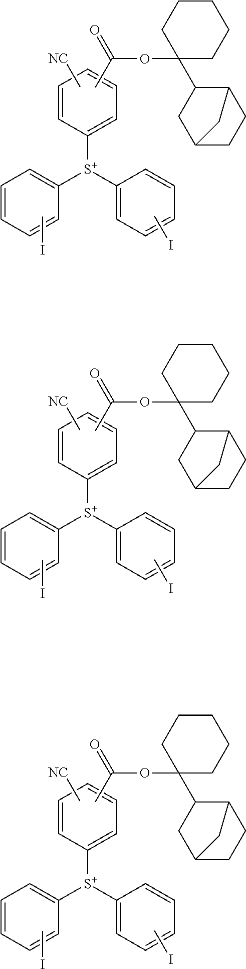

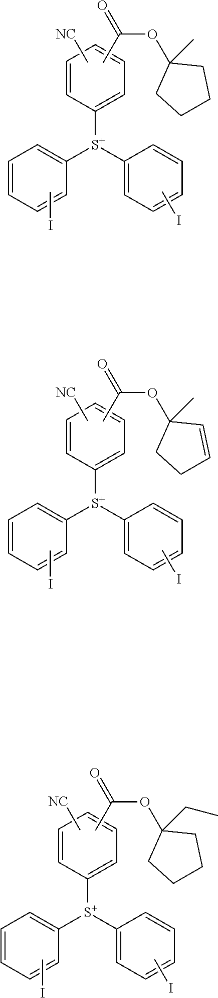

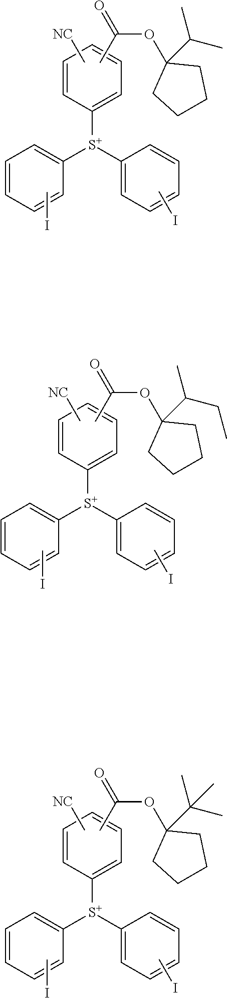

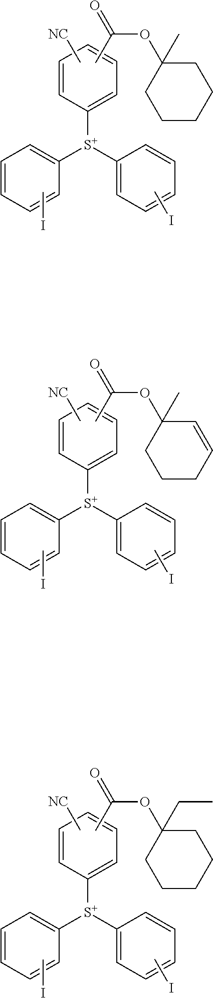

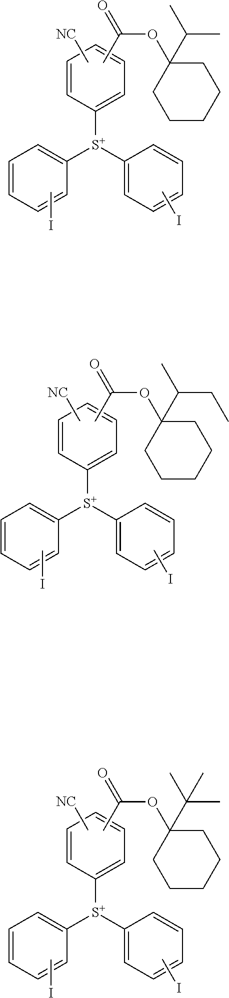

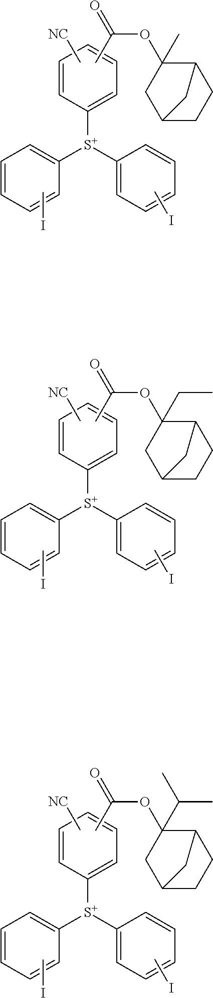

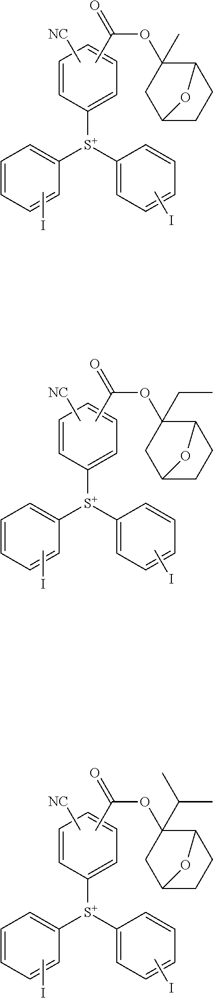

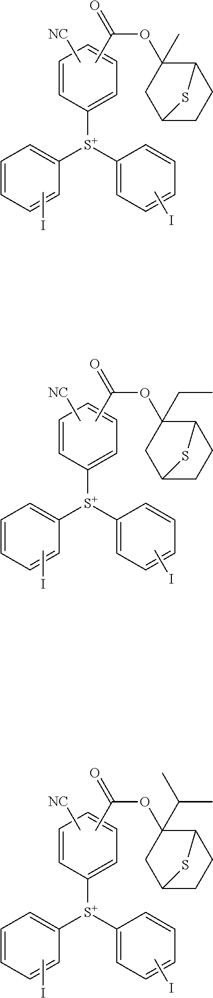

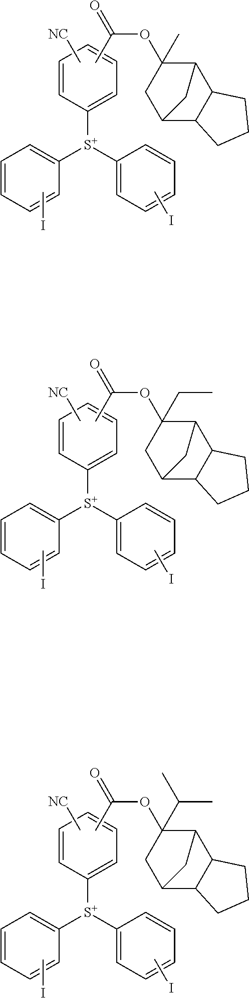

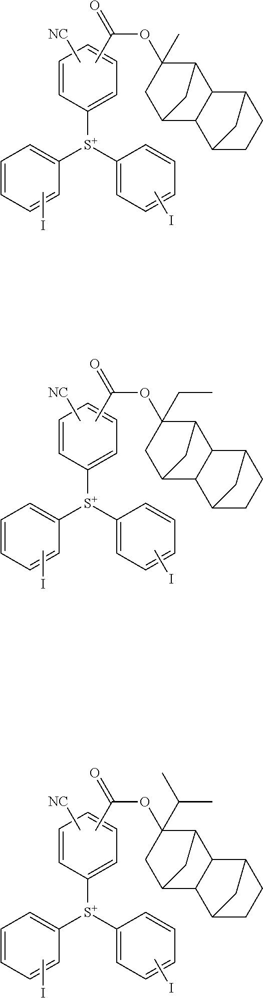

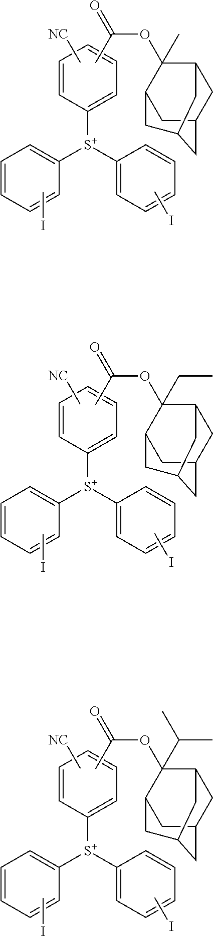

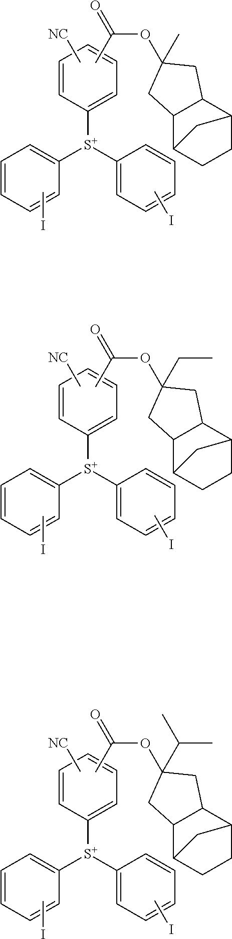

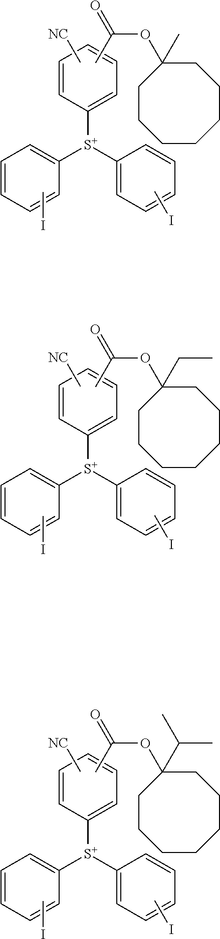

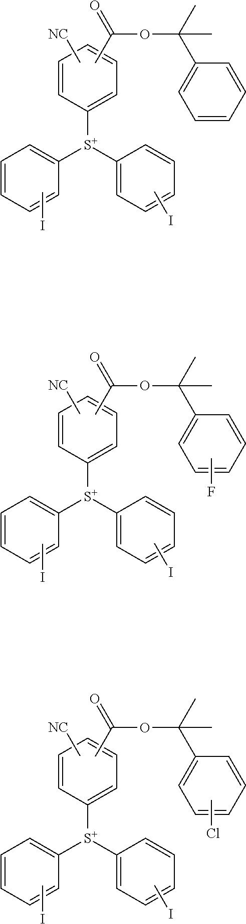

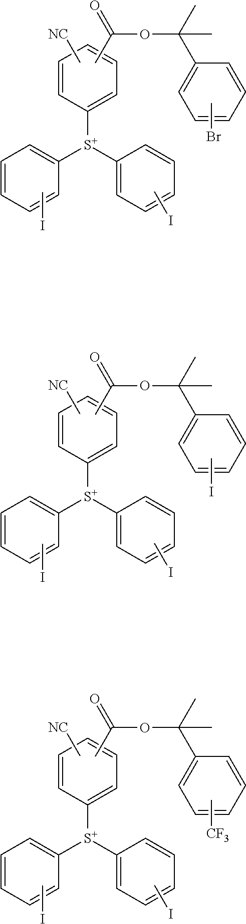

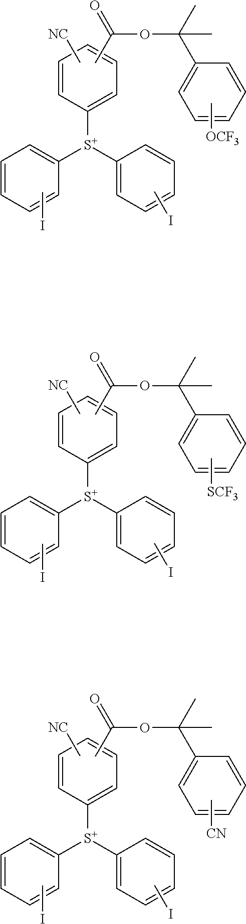

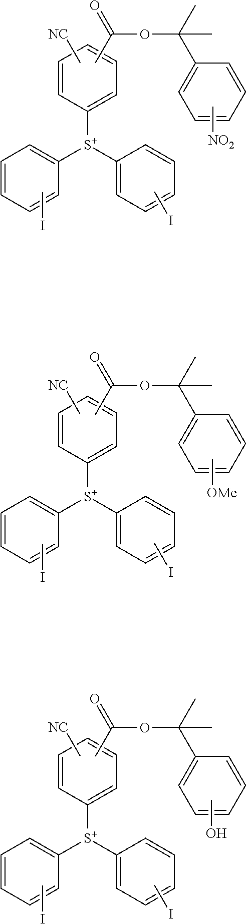

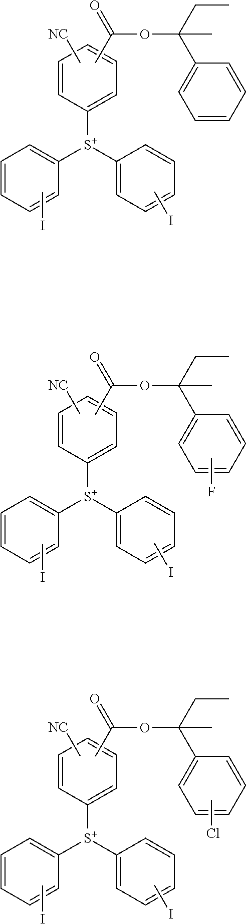

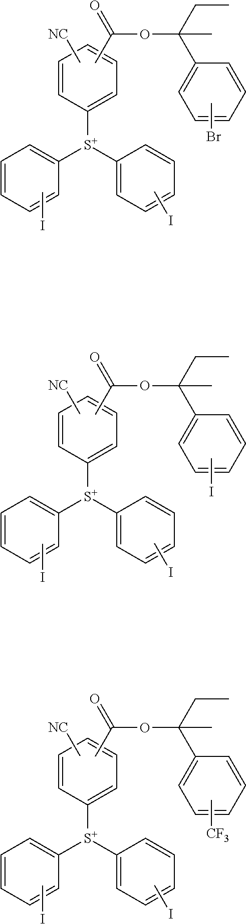

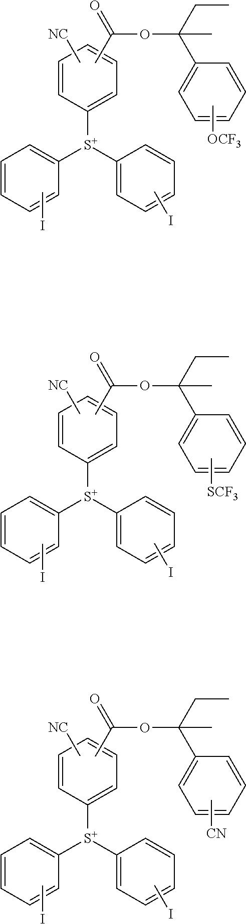

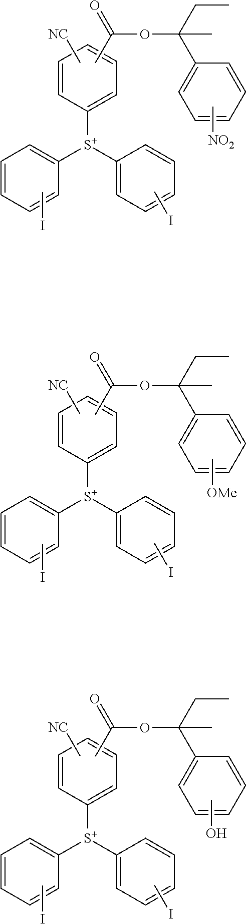

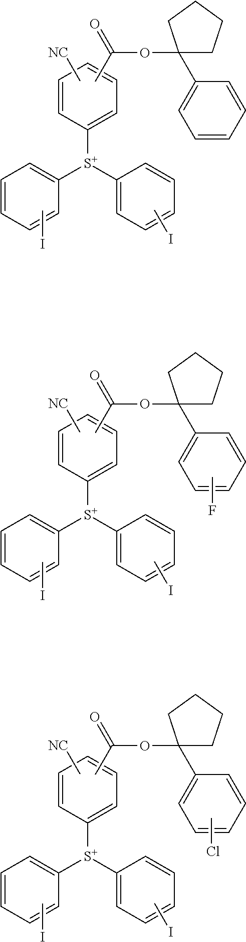

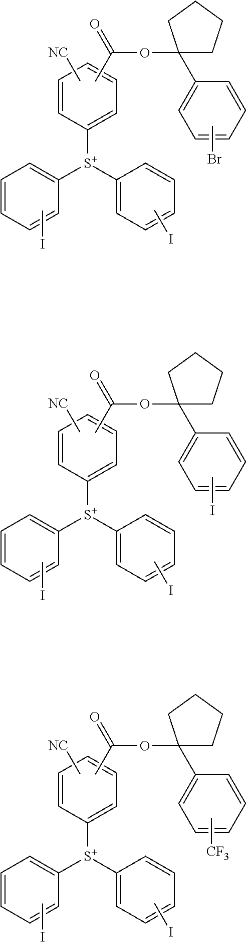

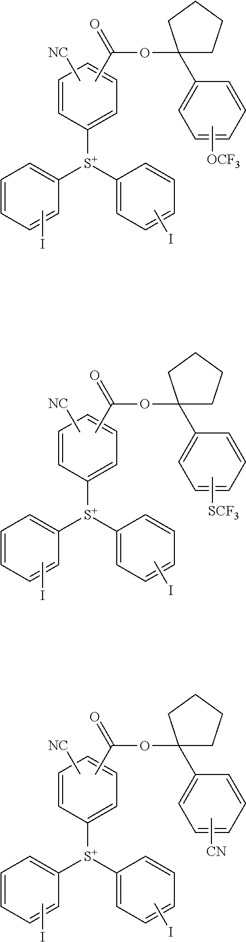

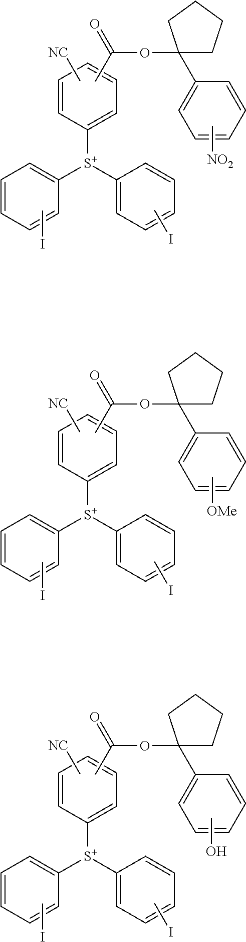

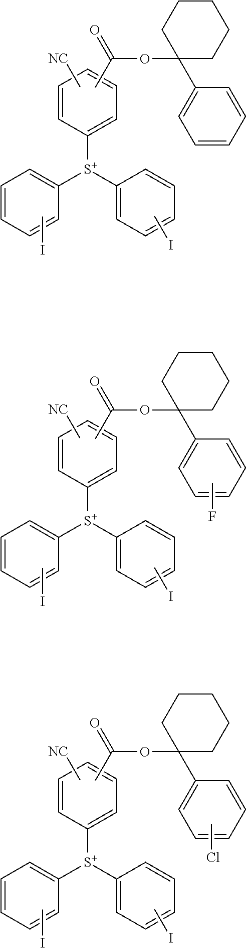

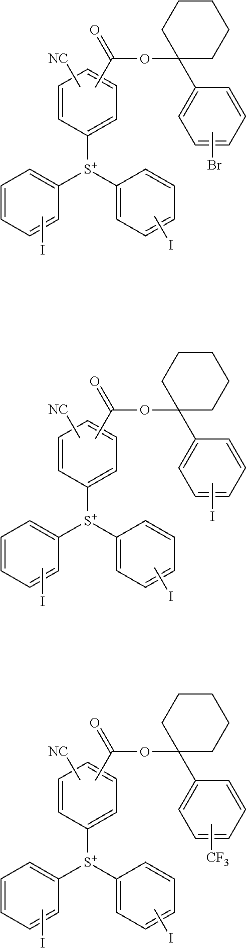

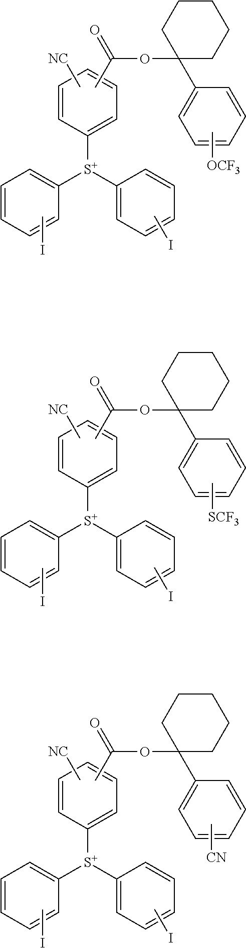

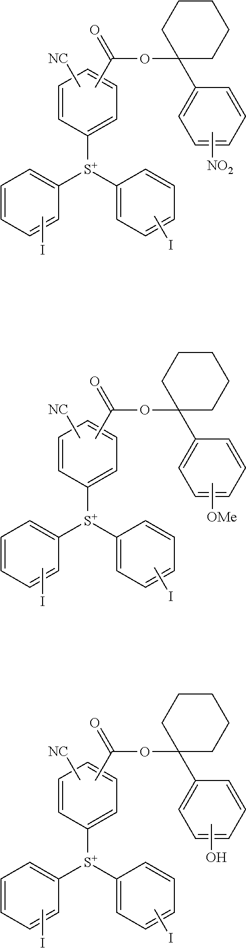

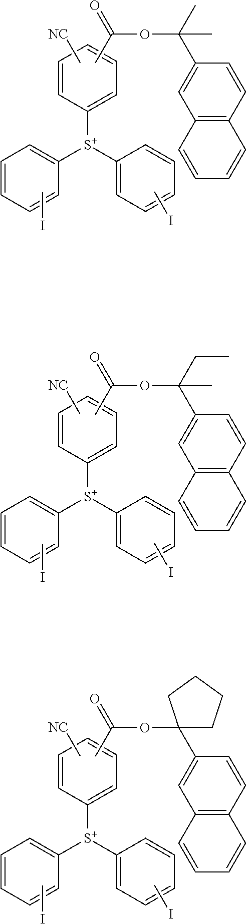

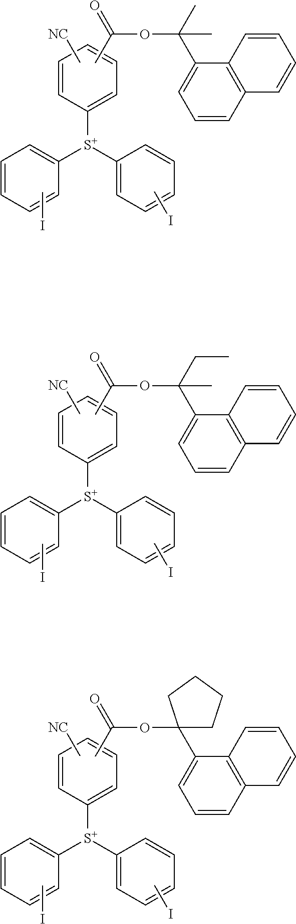

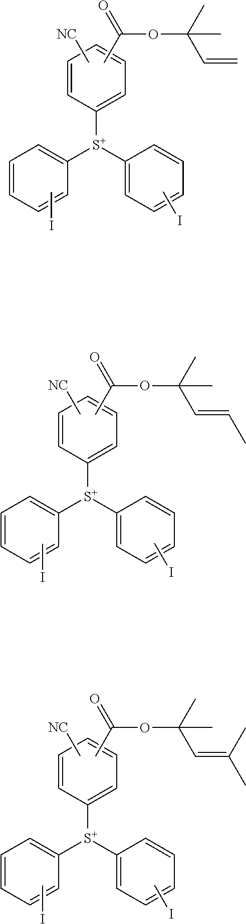

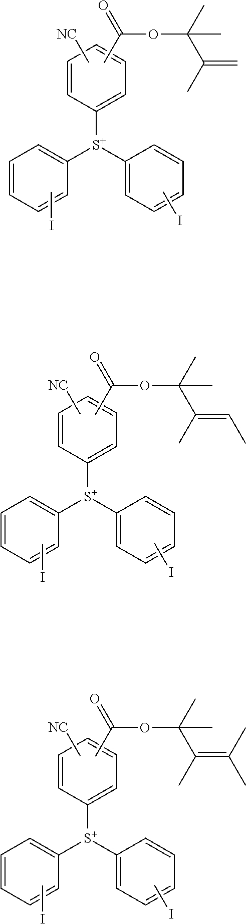

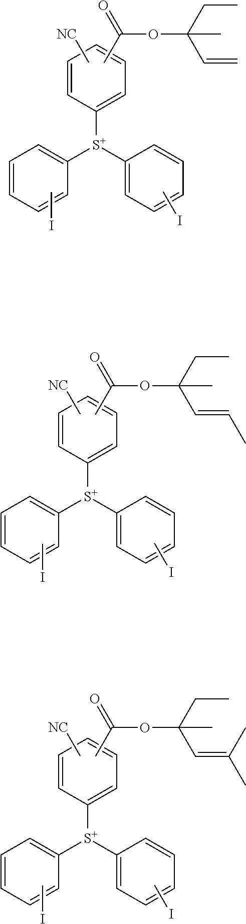

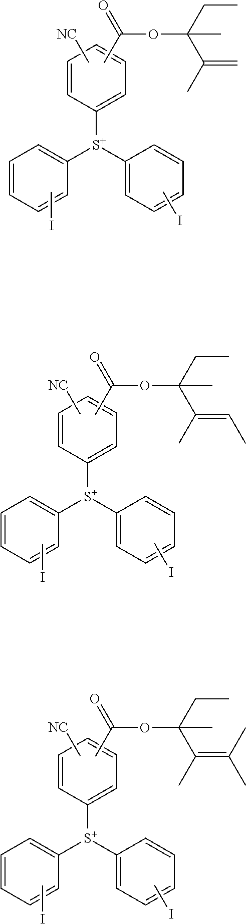

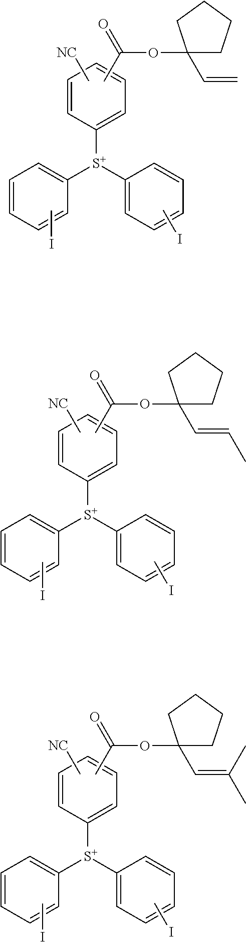

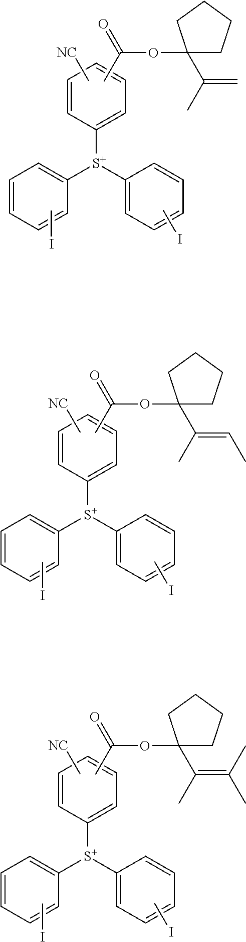

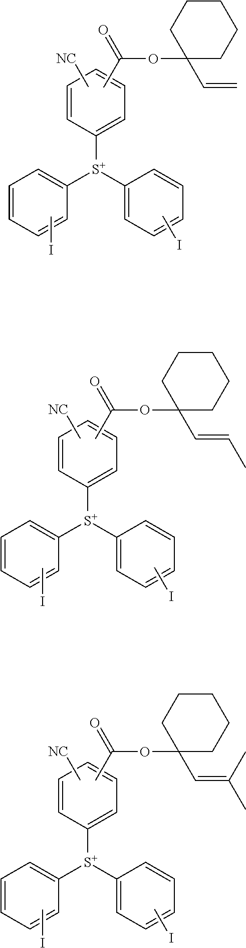

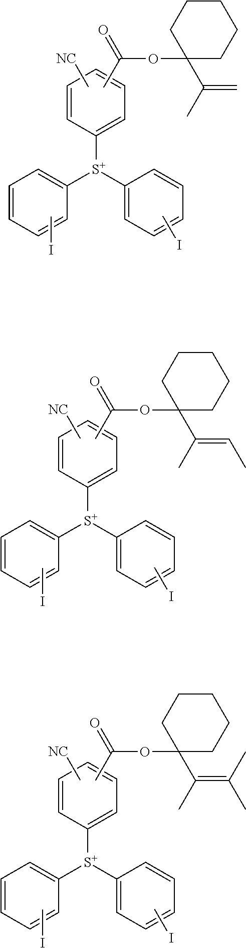

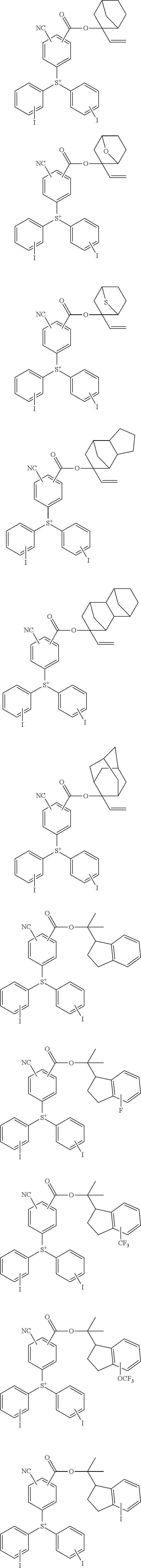

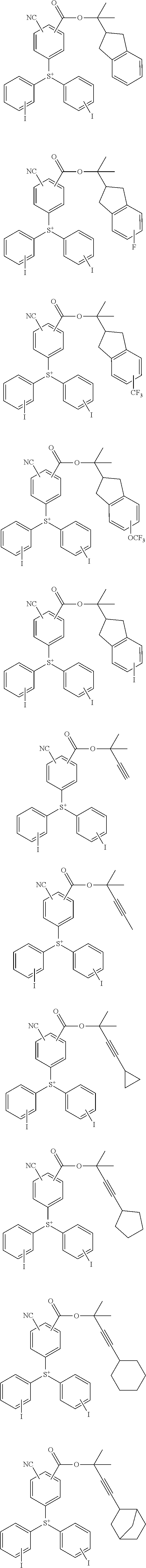

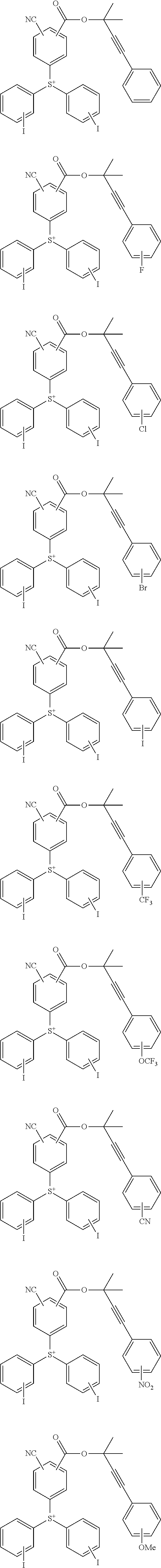

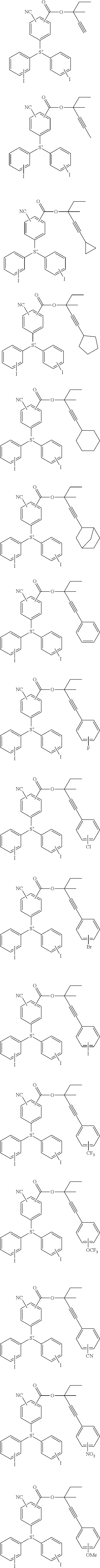

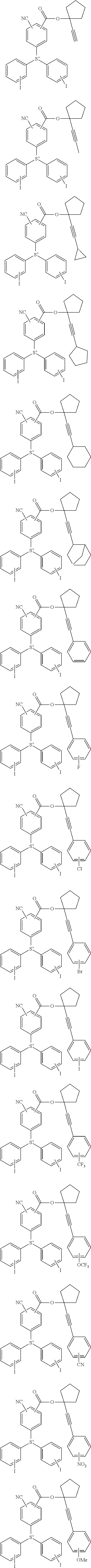

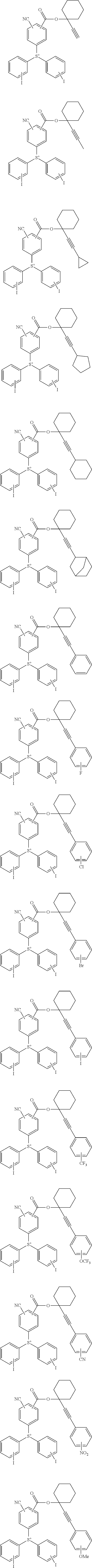

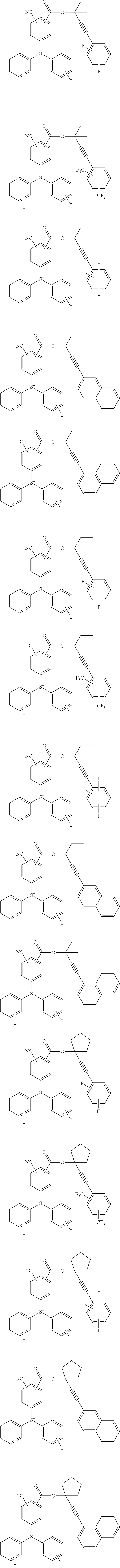

[chemical structure]

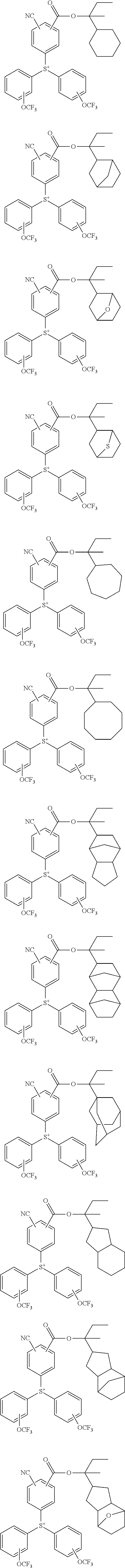

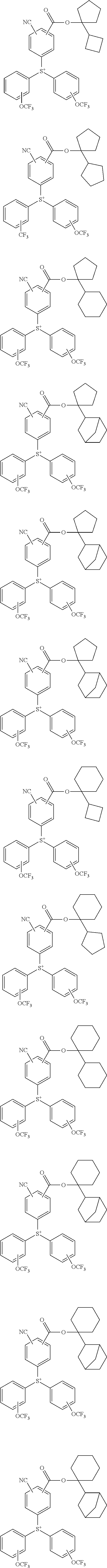

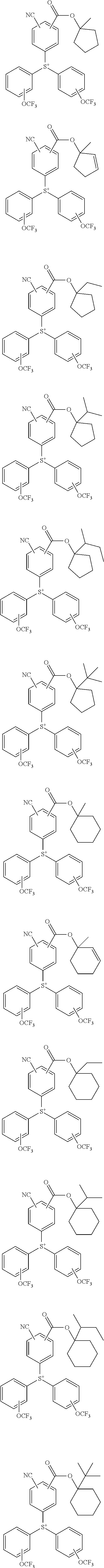

[chemical structure]

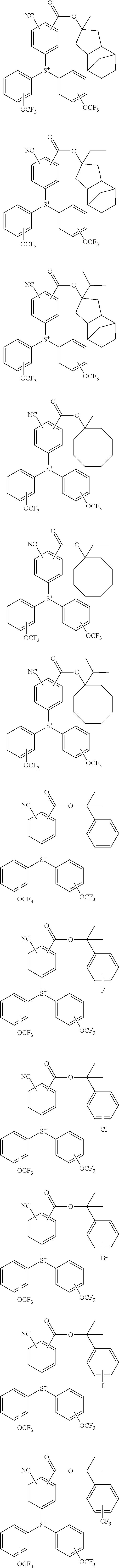

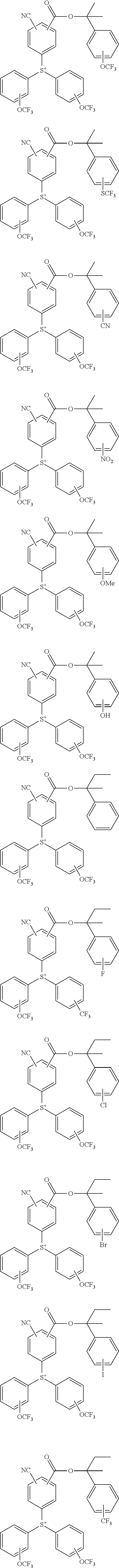

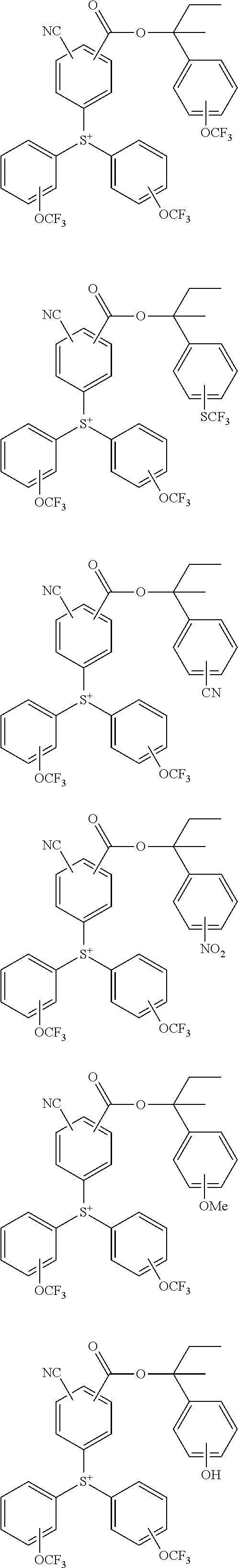

[chemical structure]

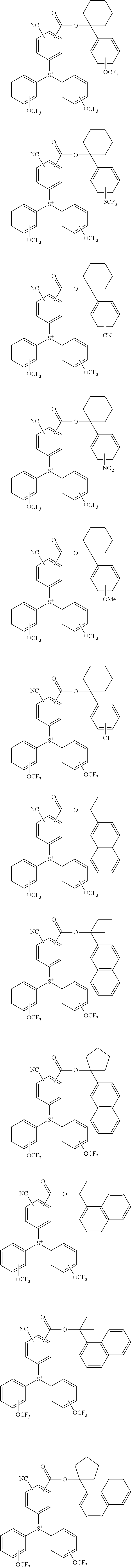

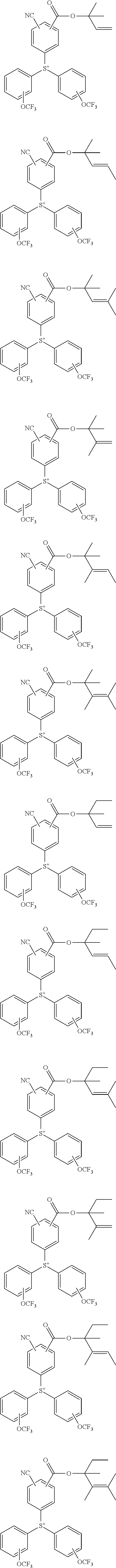

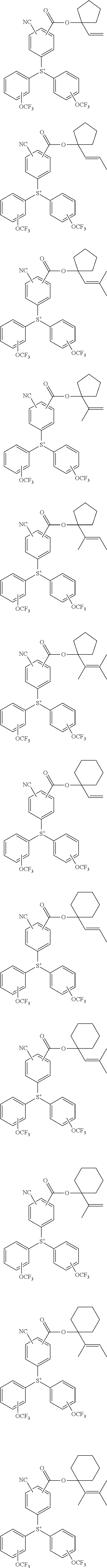

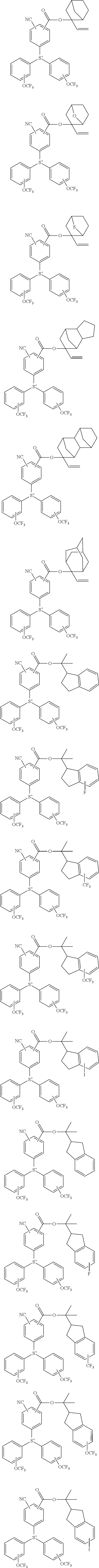

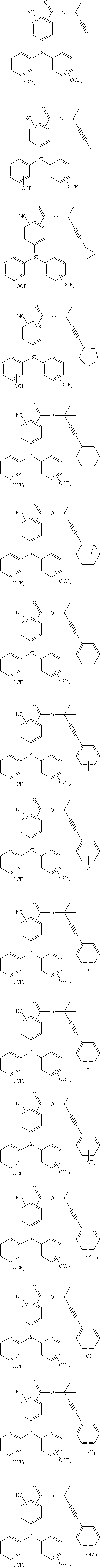

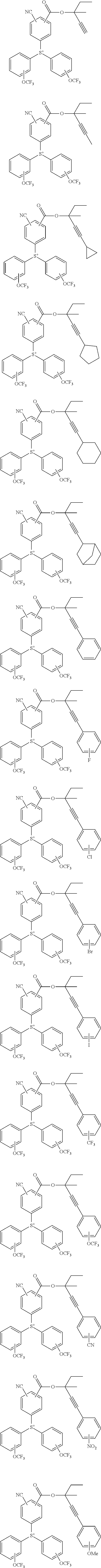

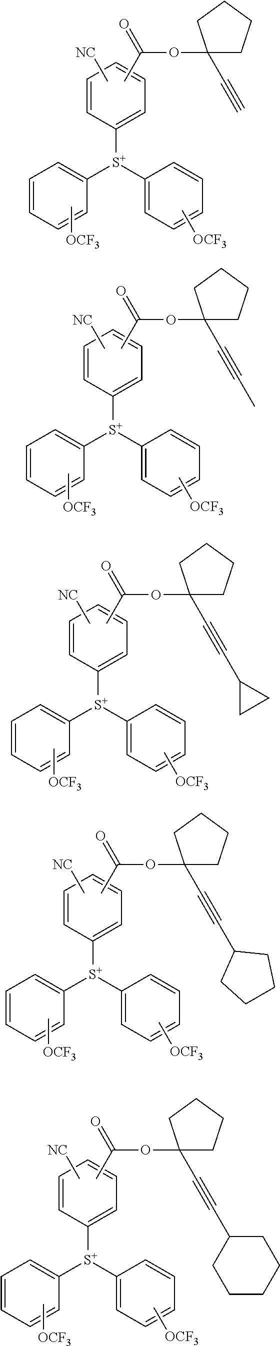

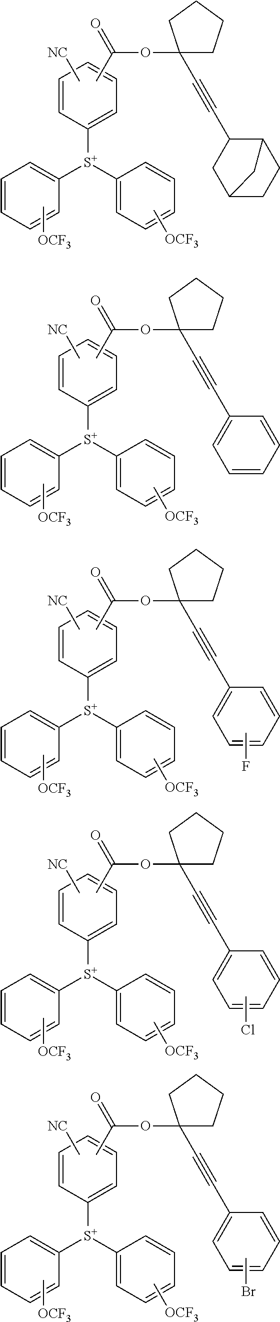

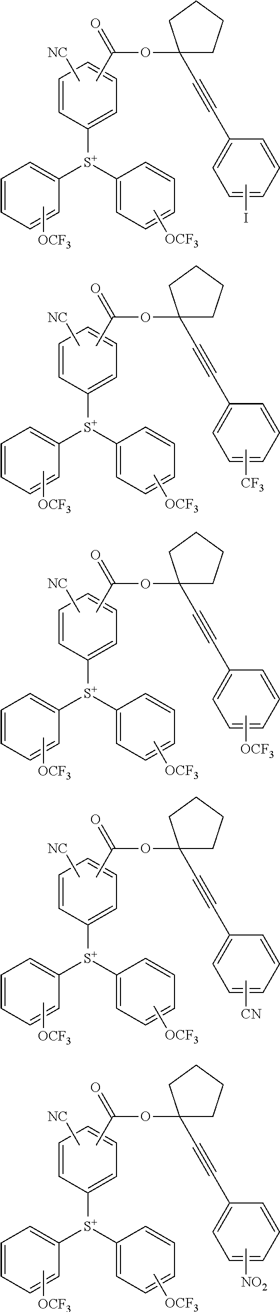

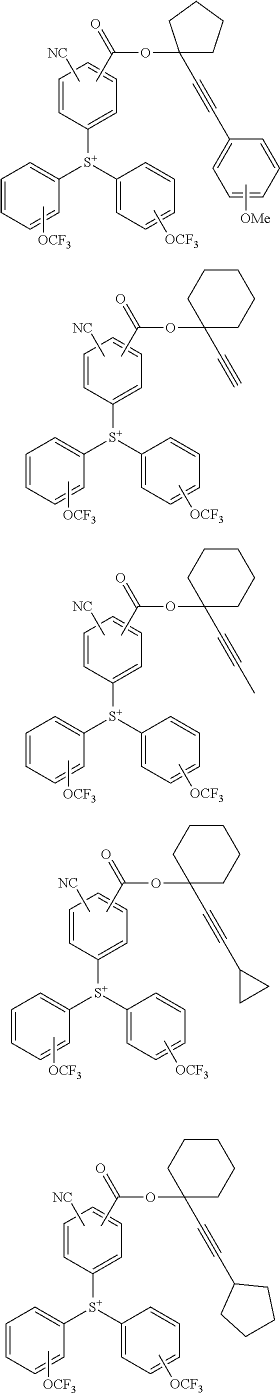

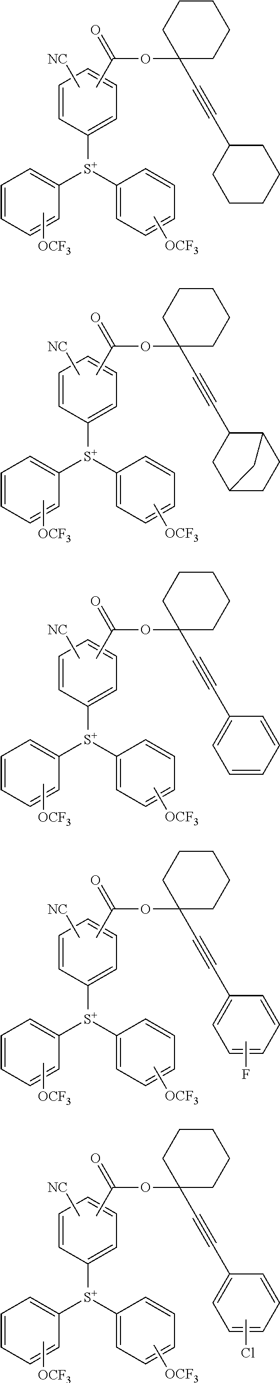

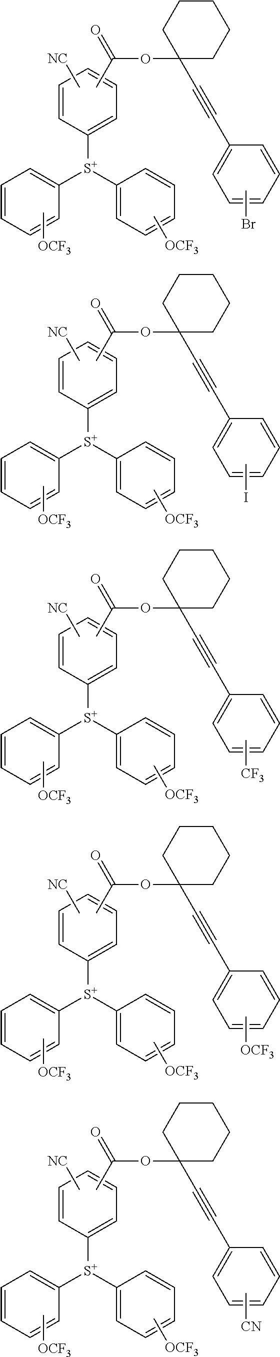

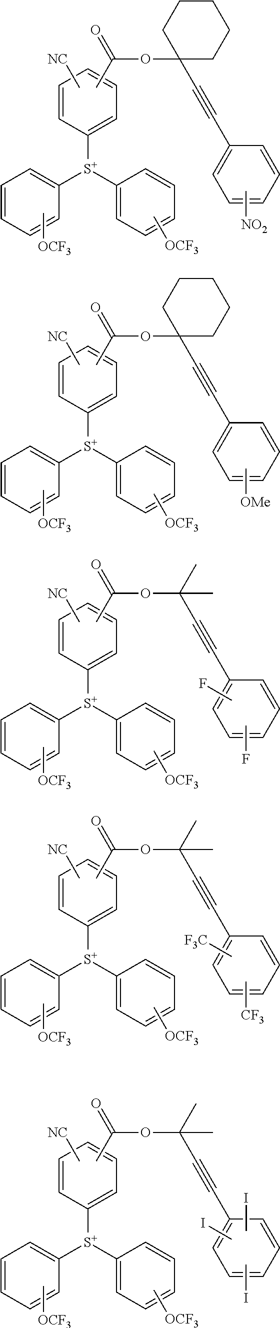

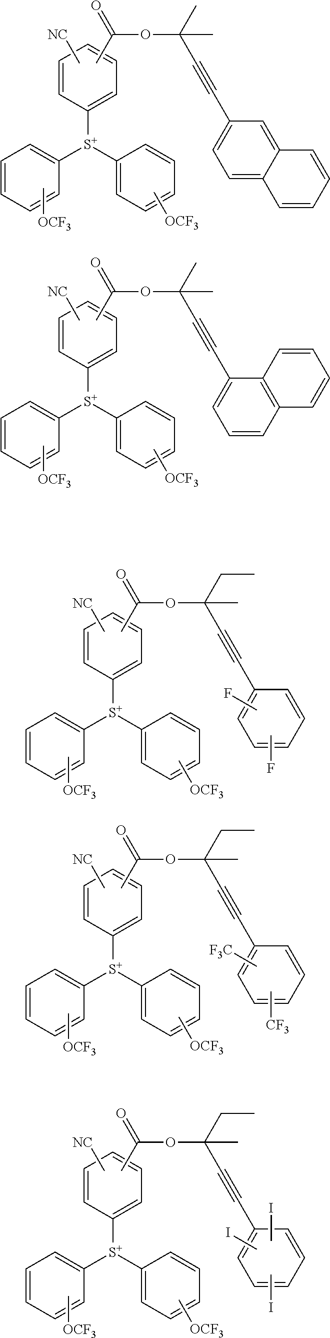

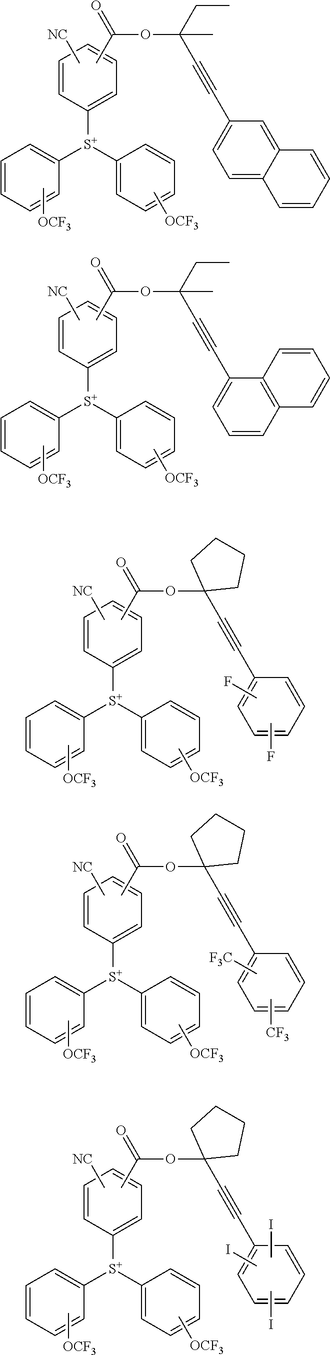

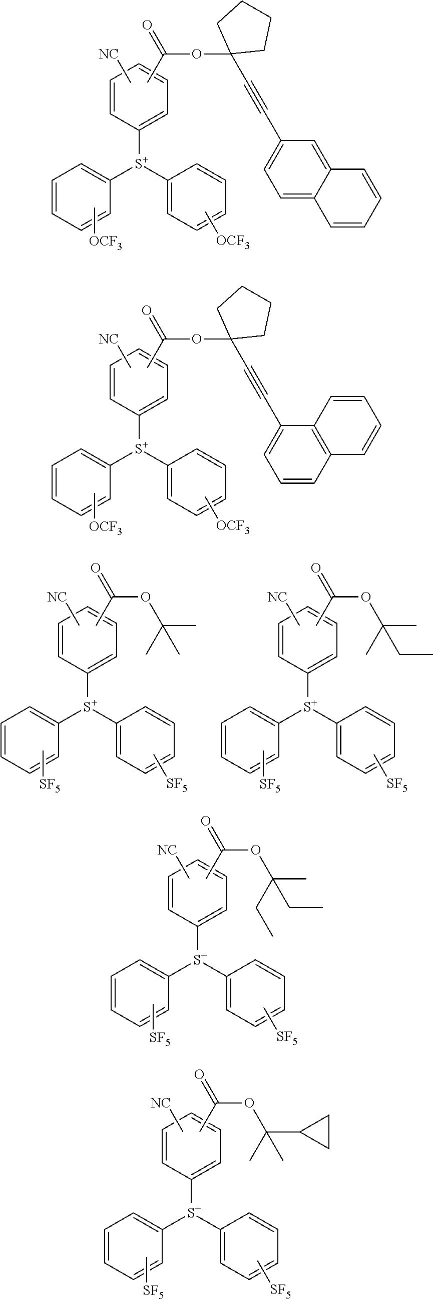

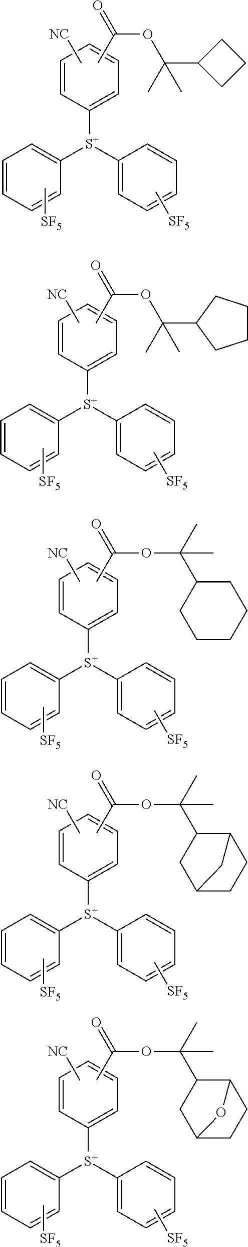

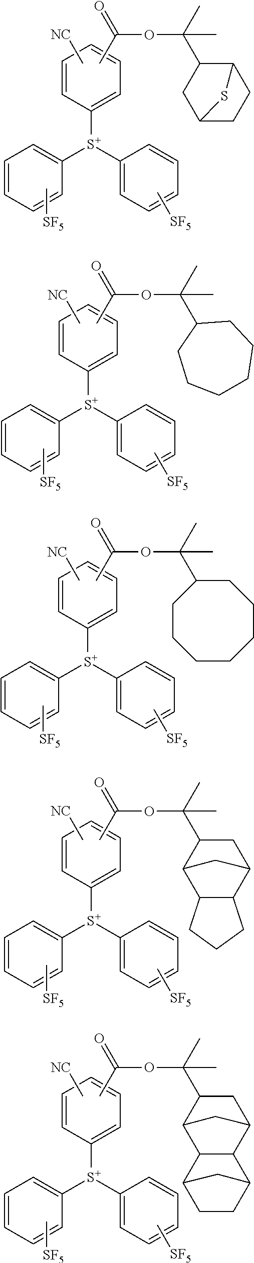

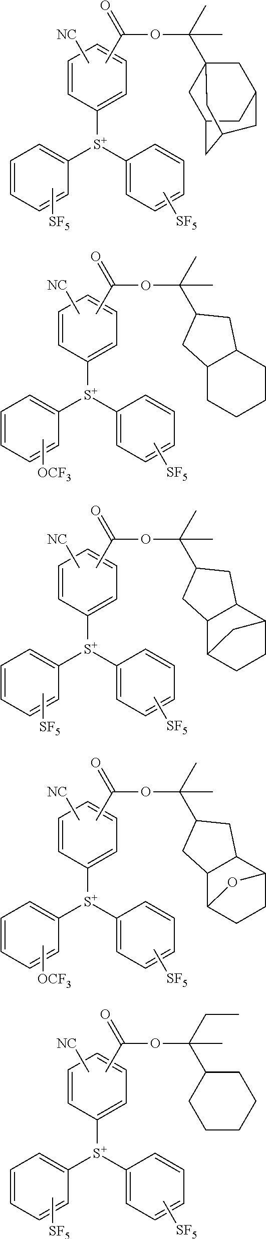

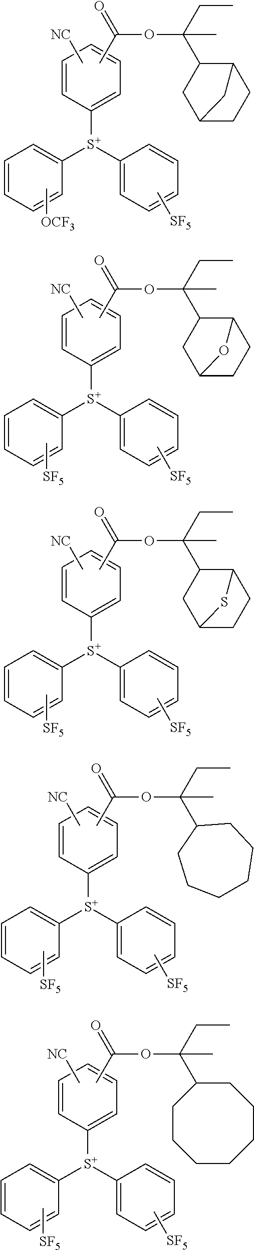

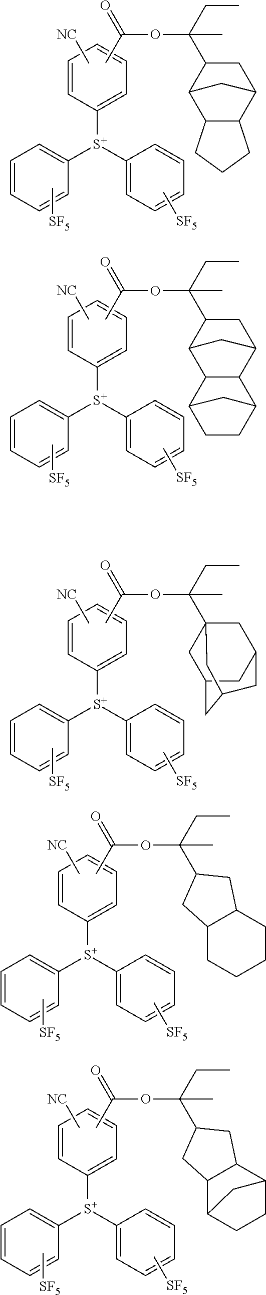

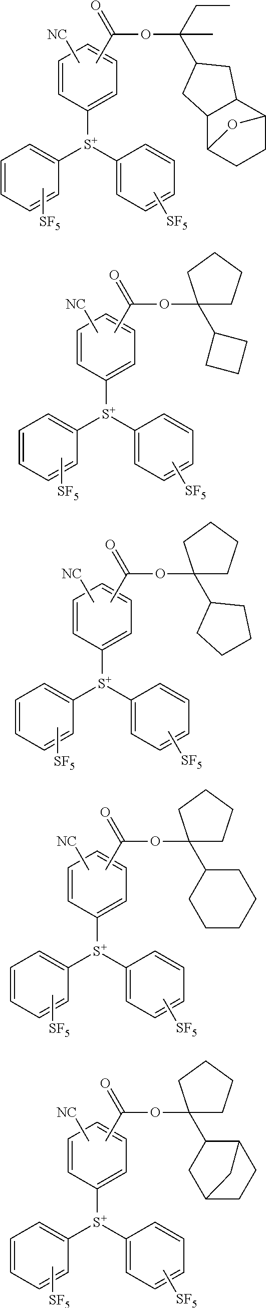

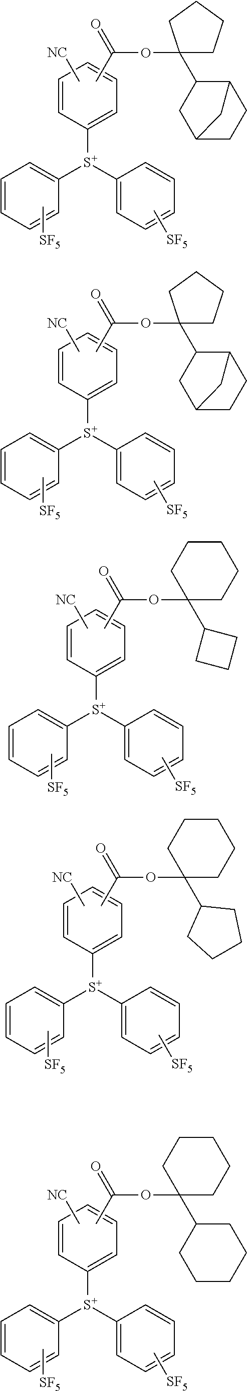

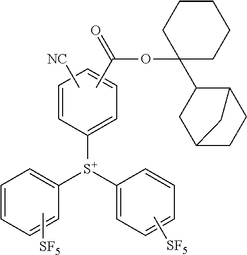

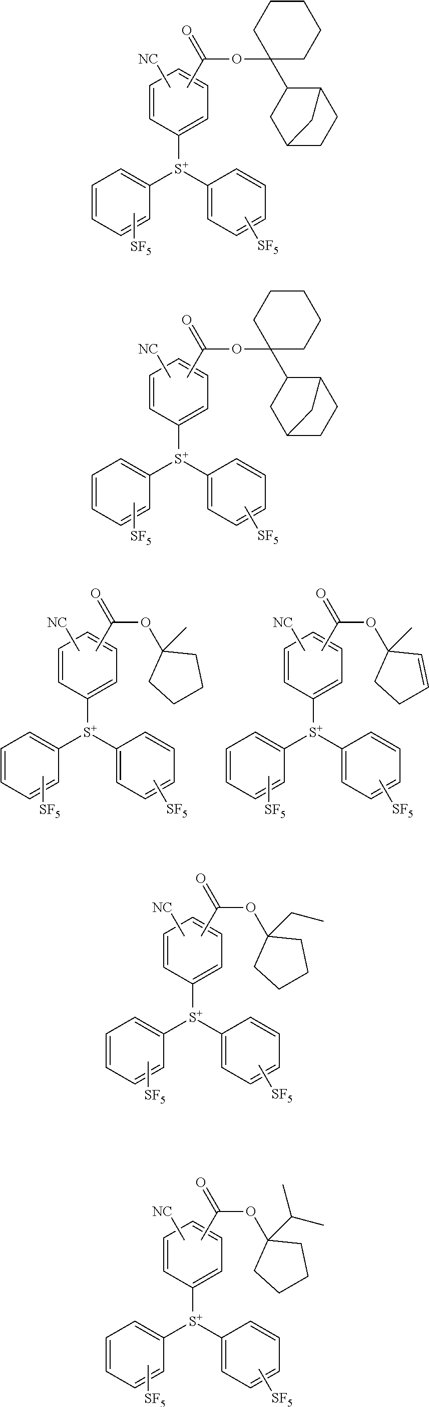

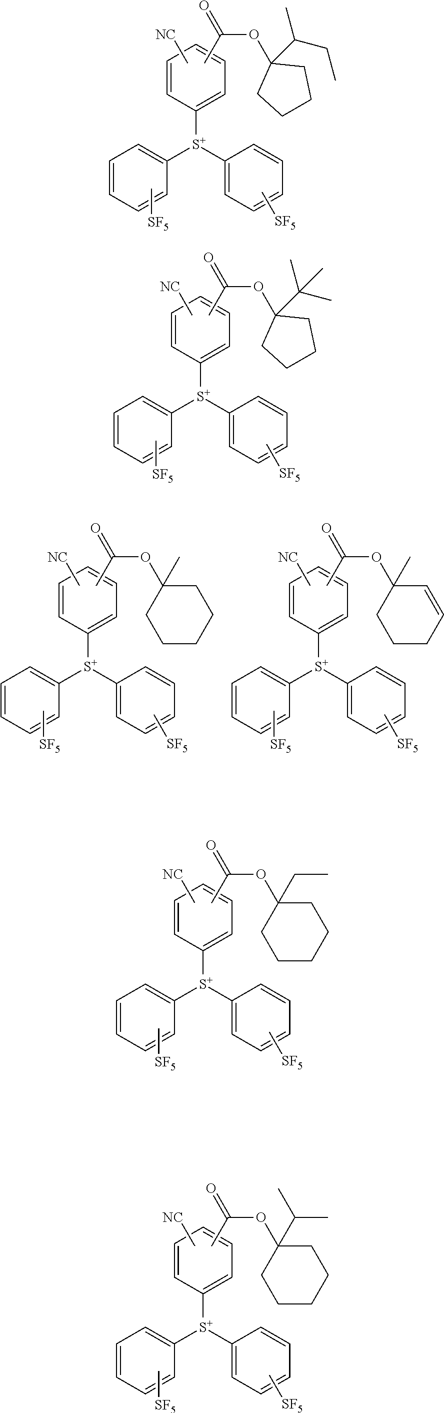

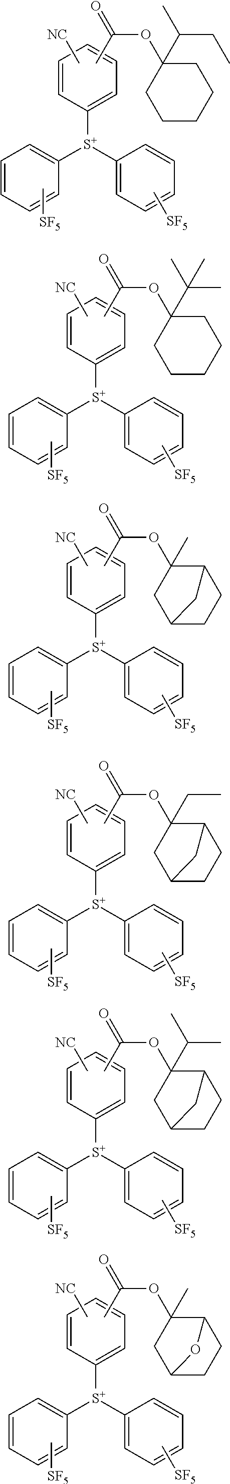

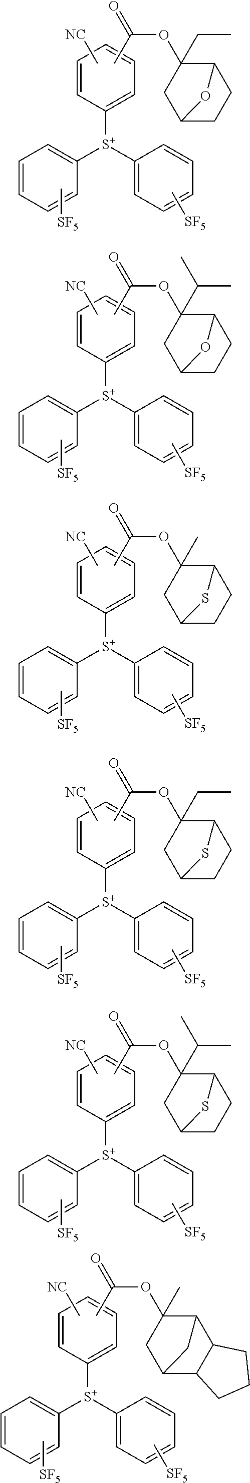

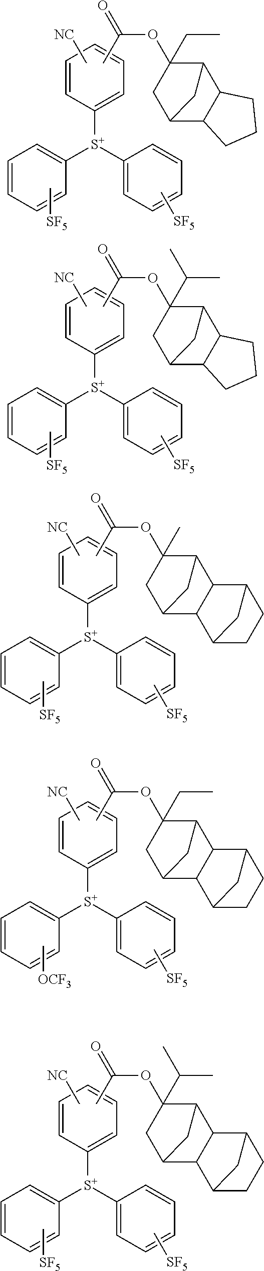

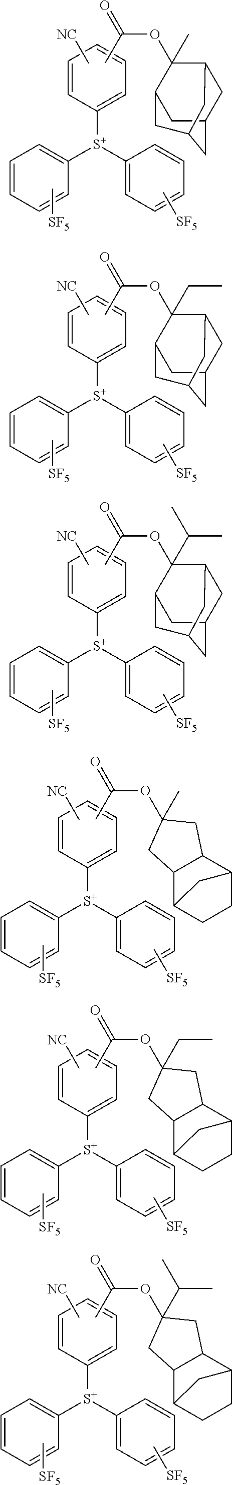

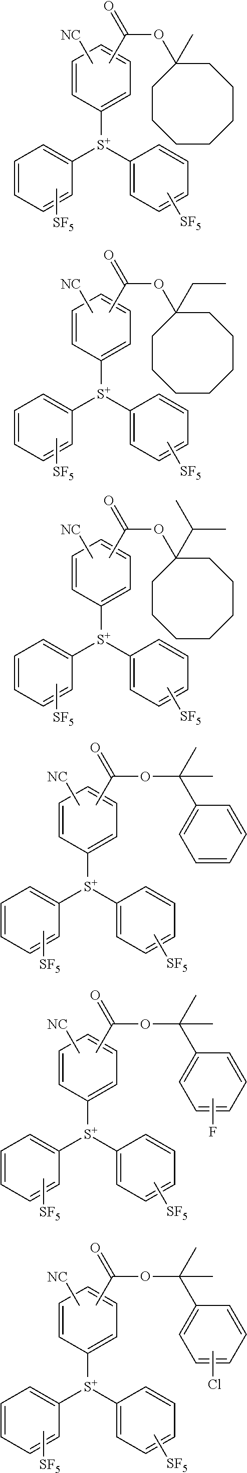

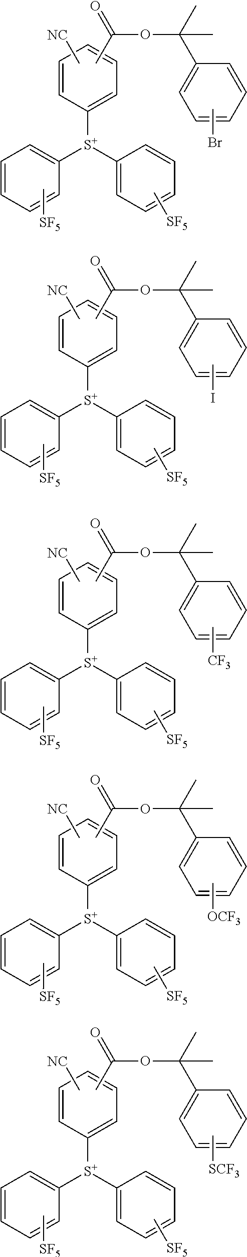

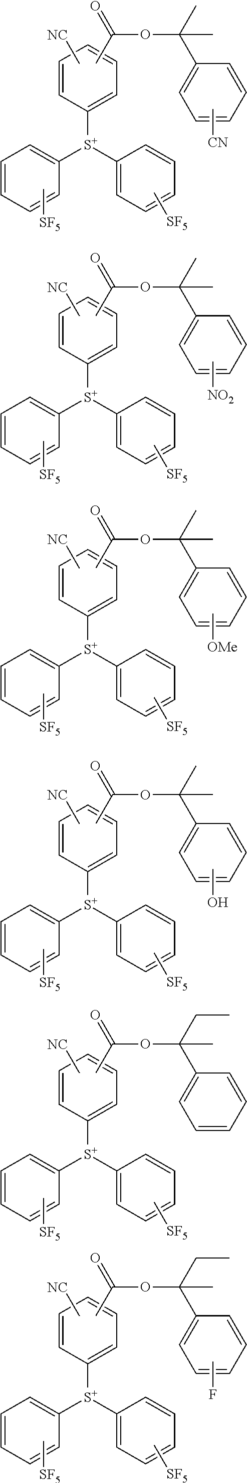

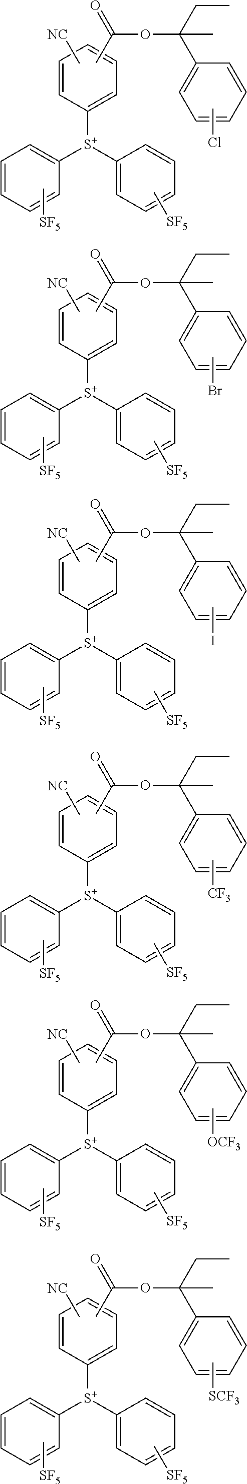

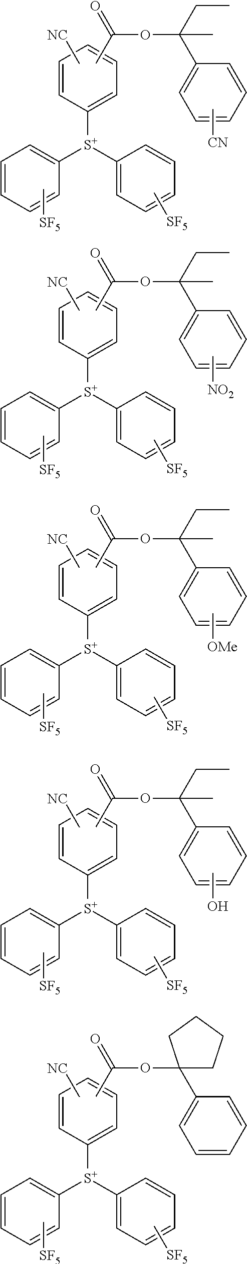

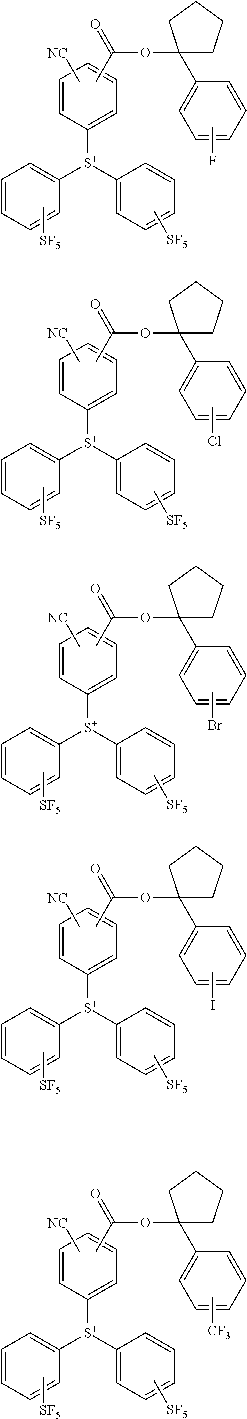

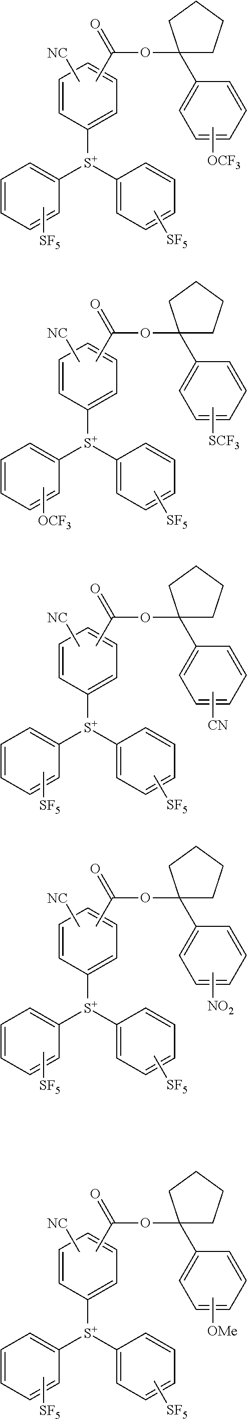

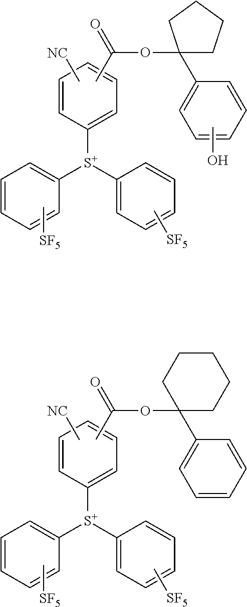

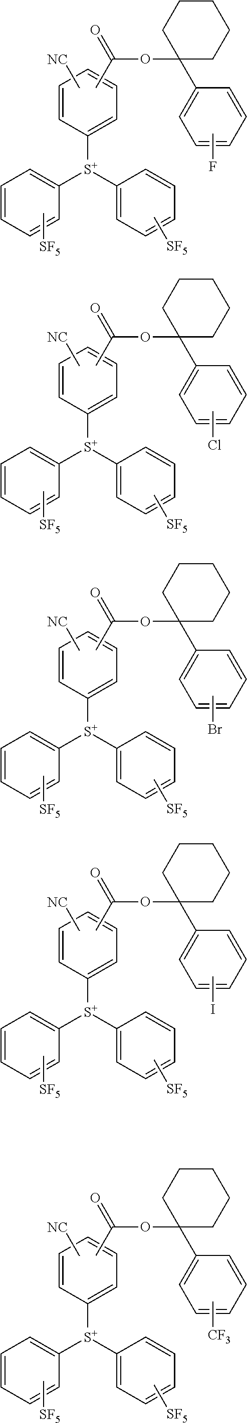

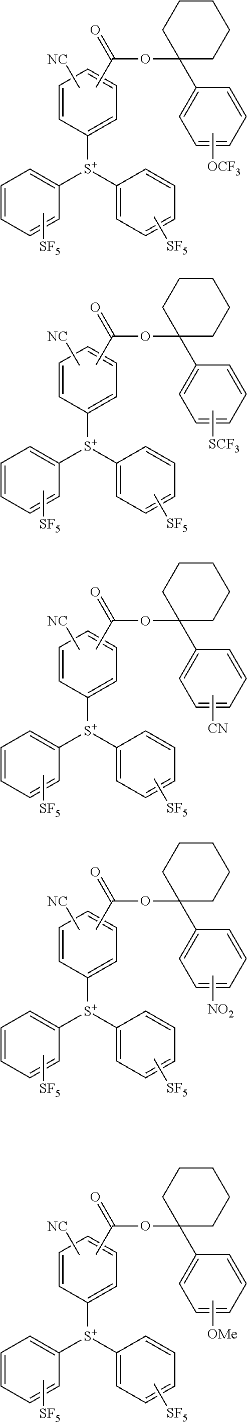

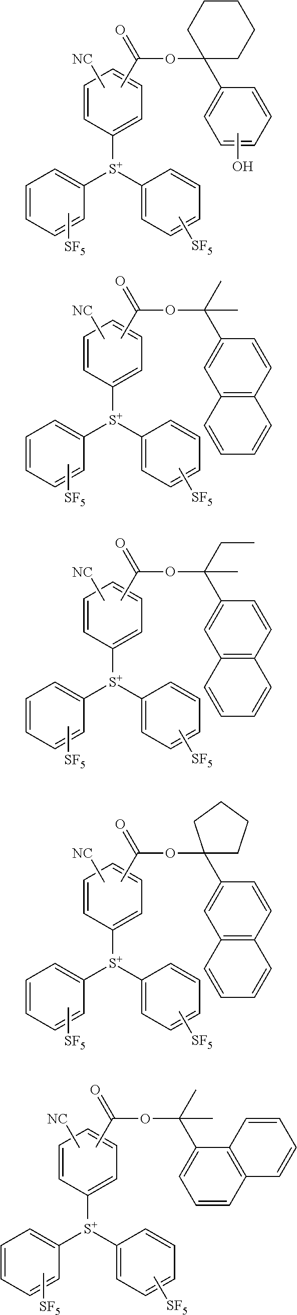

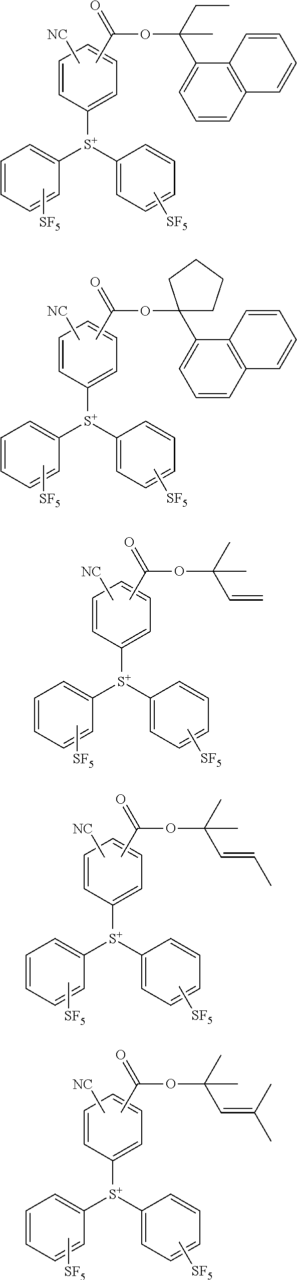

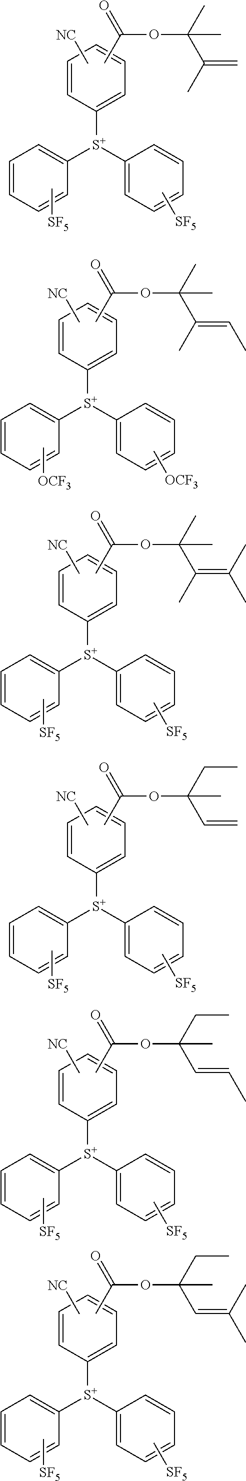

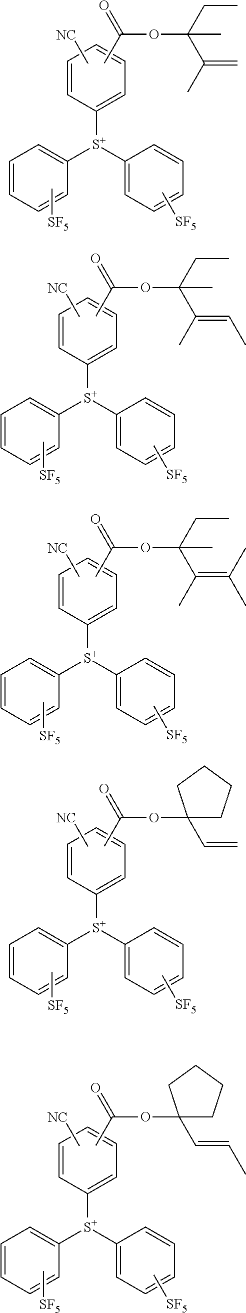

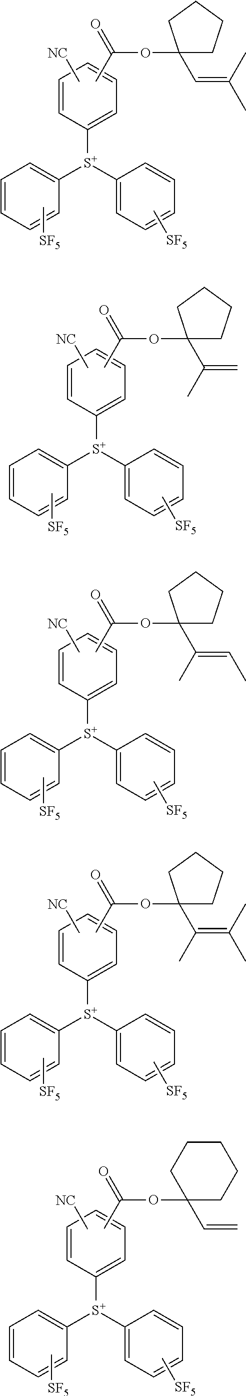

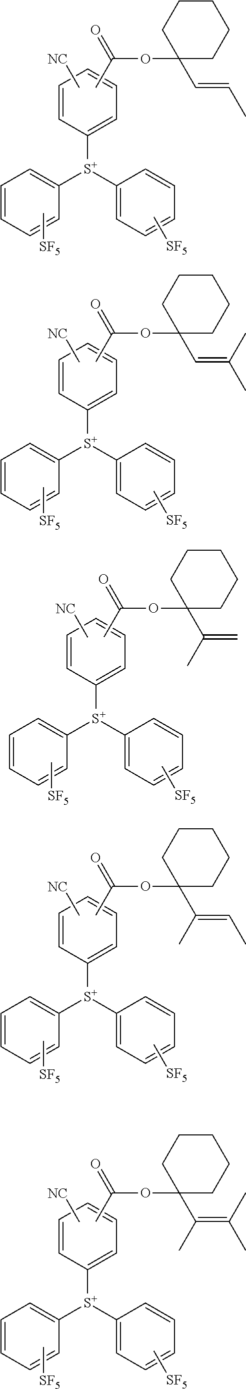

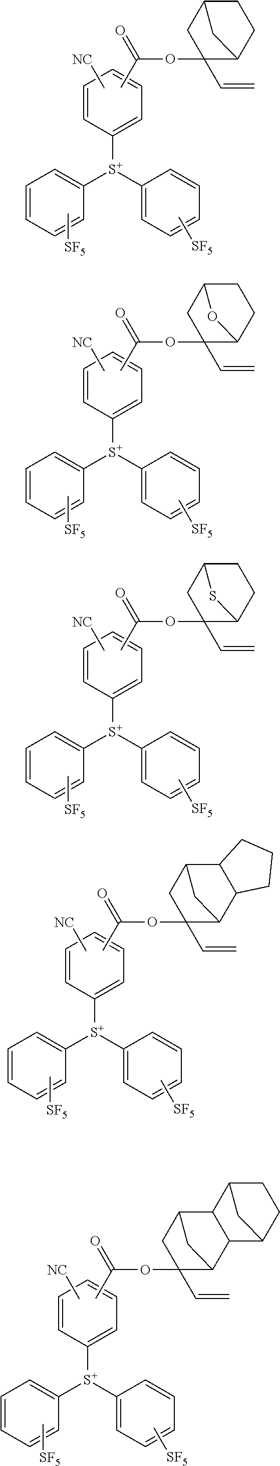

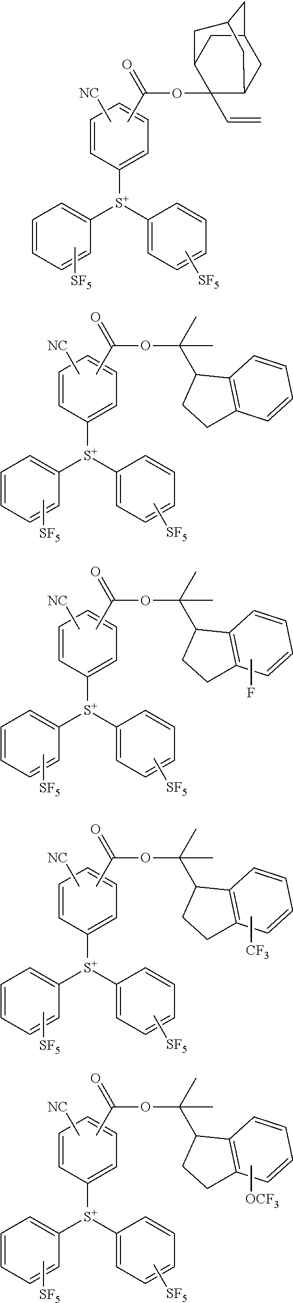

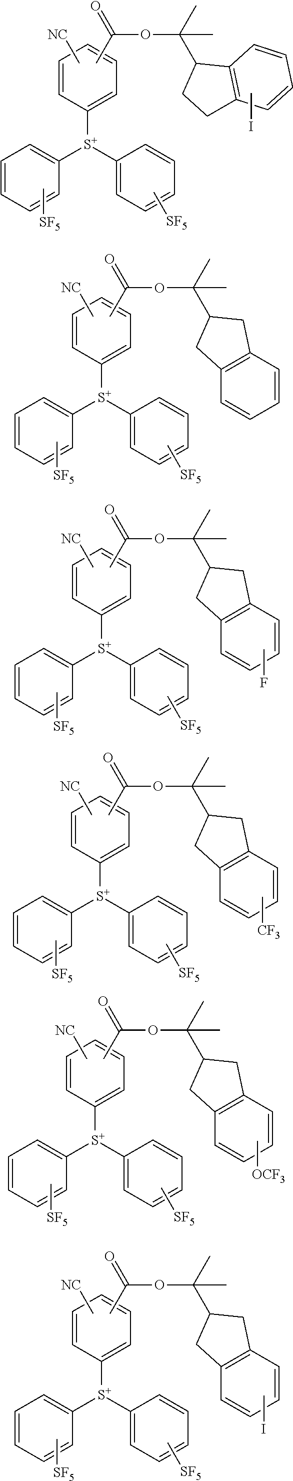

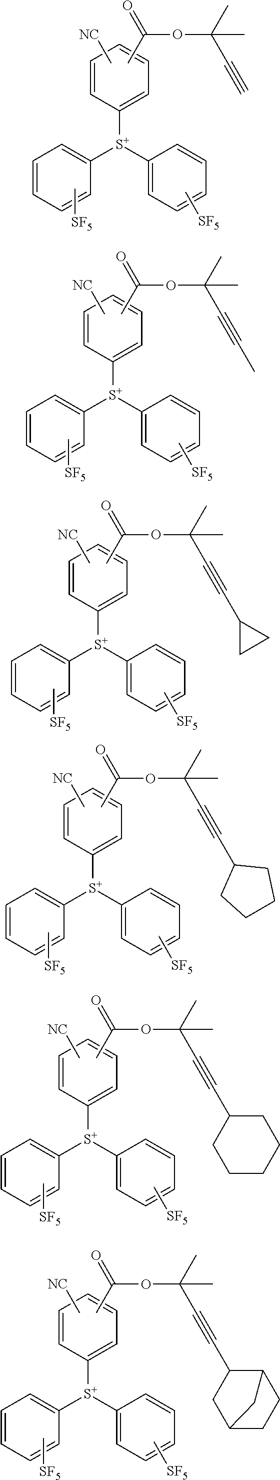

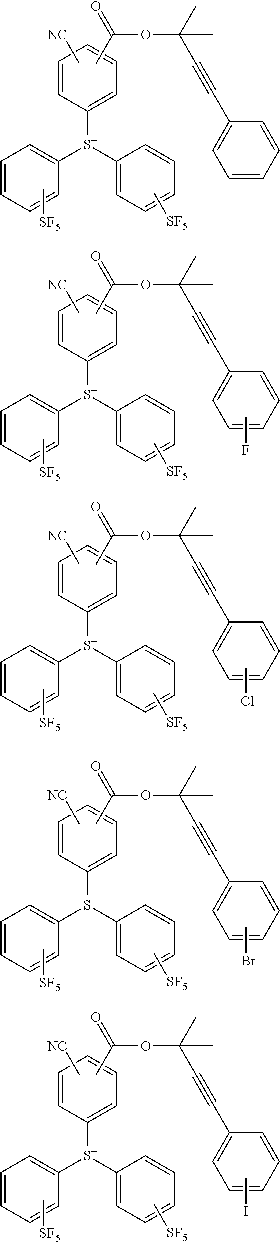

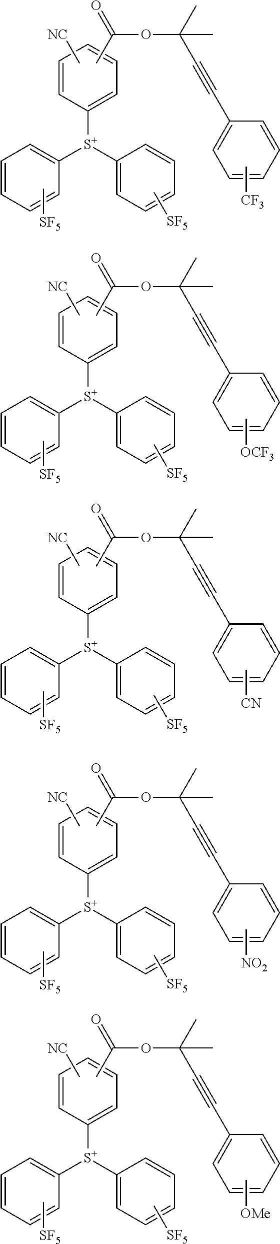

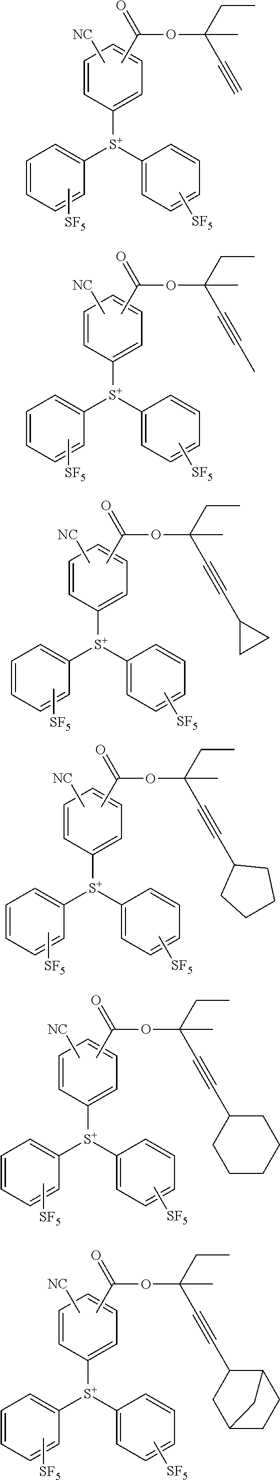

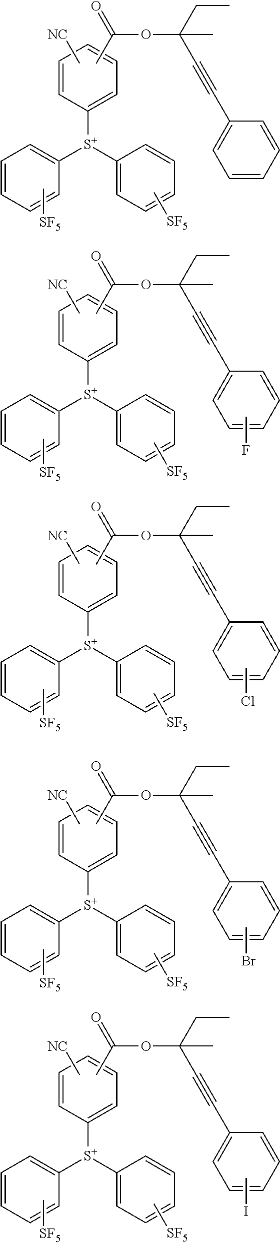

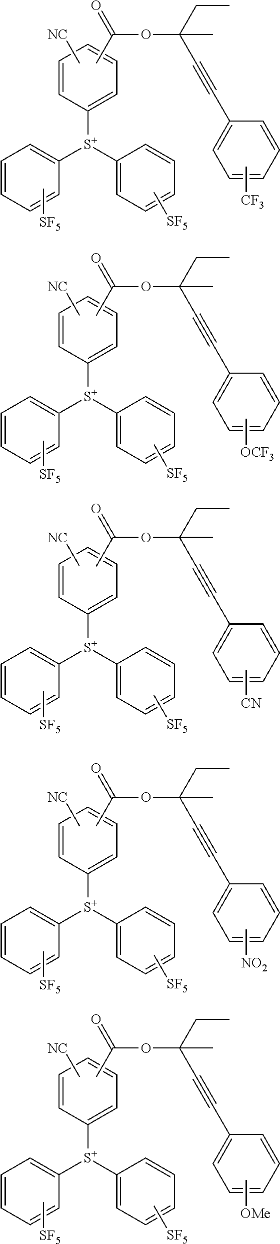

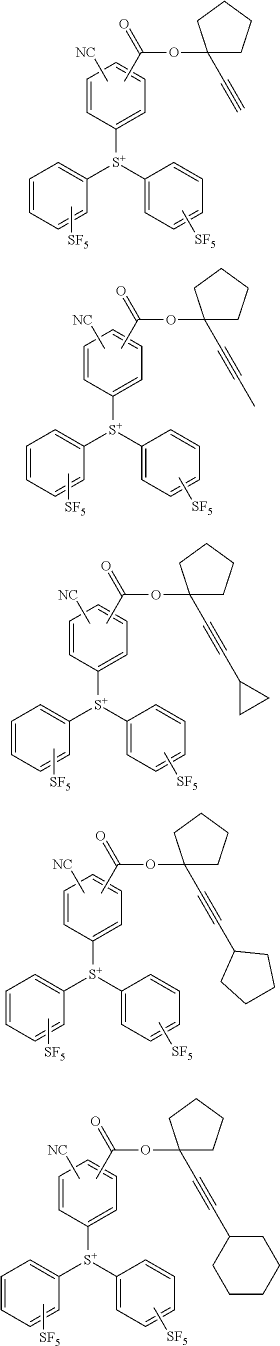

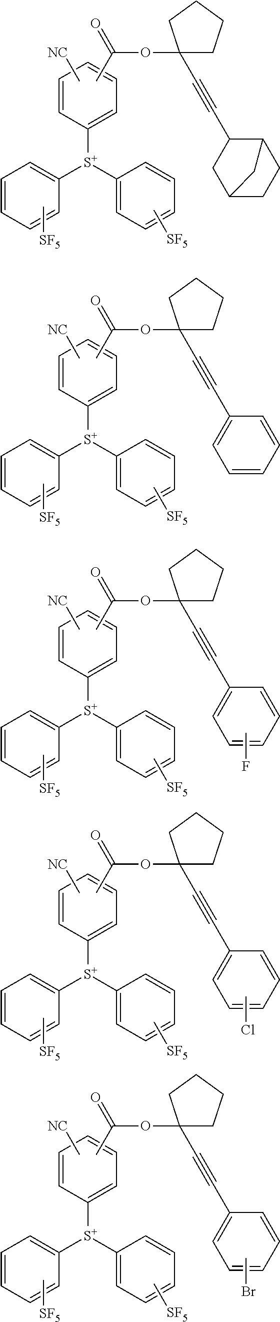

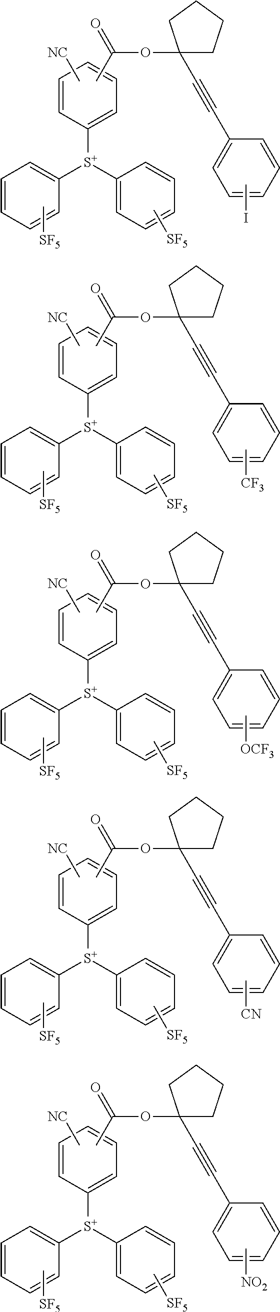

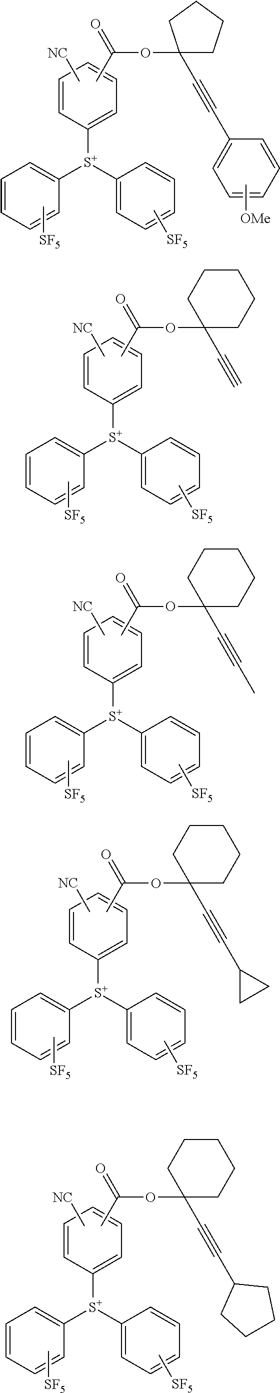

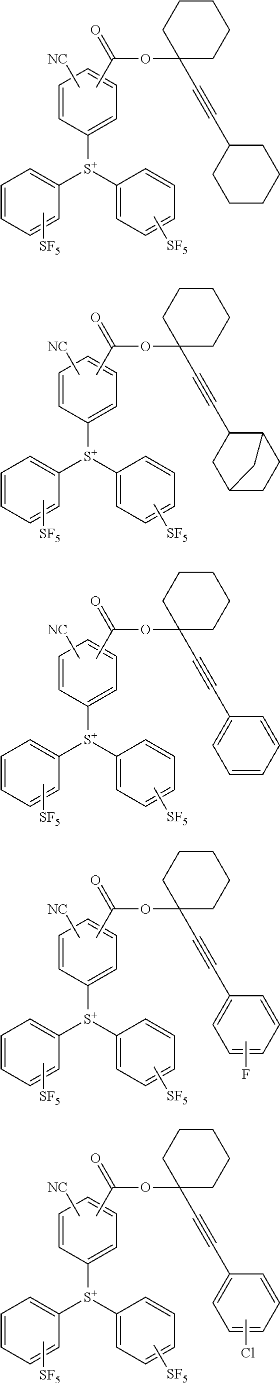

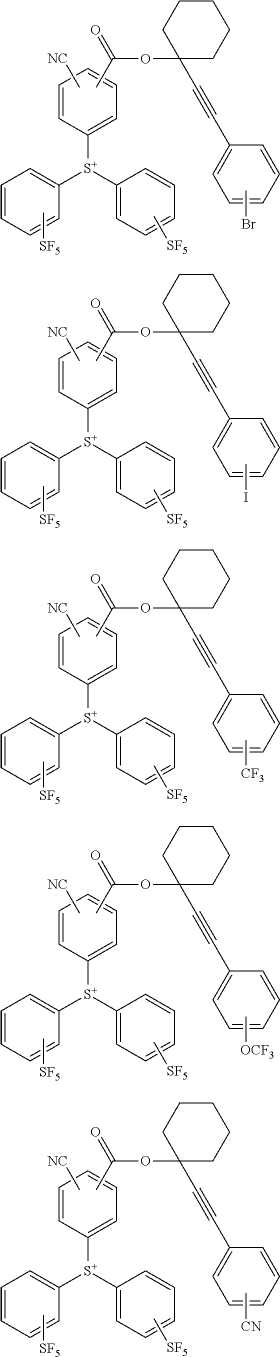

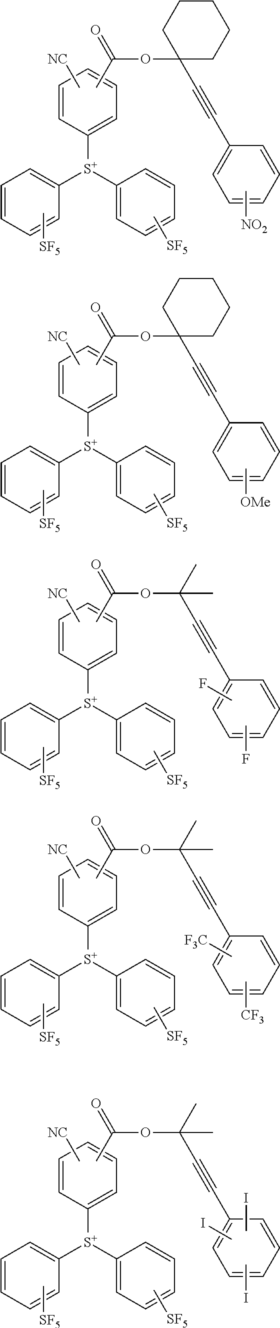

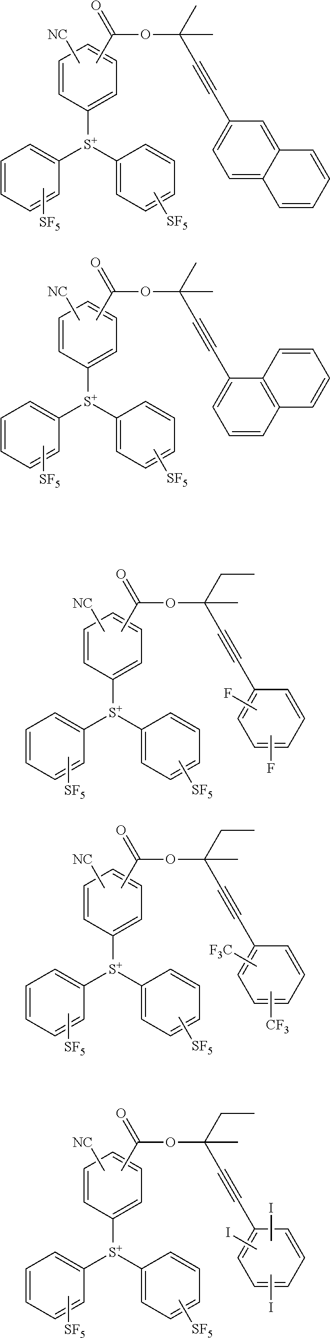

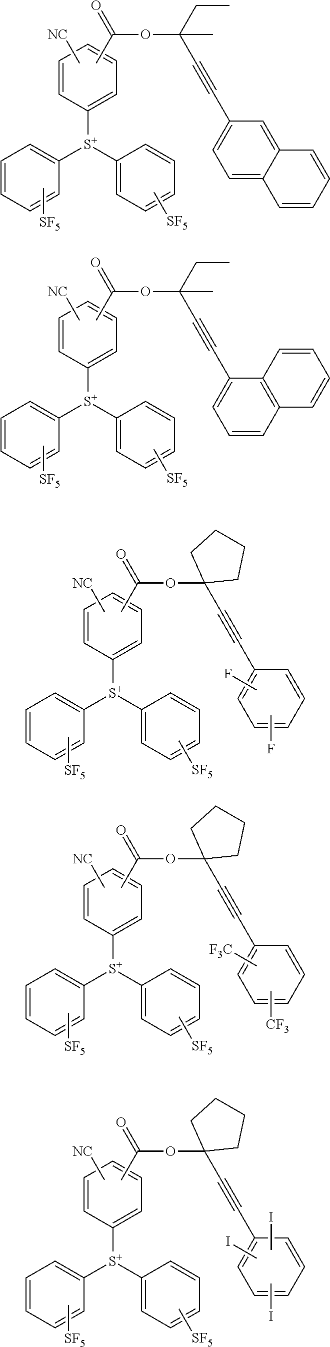

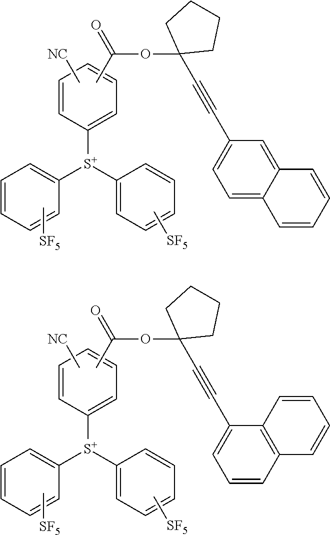

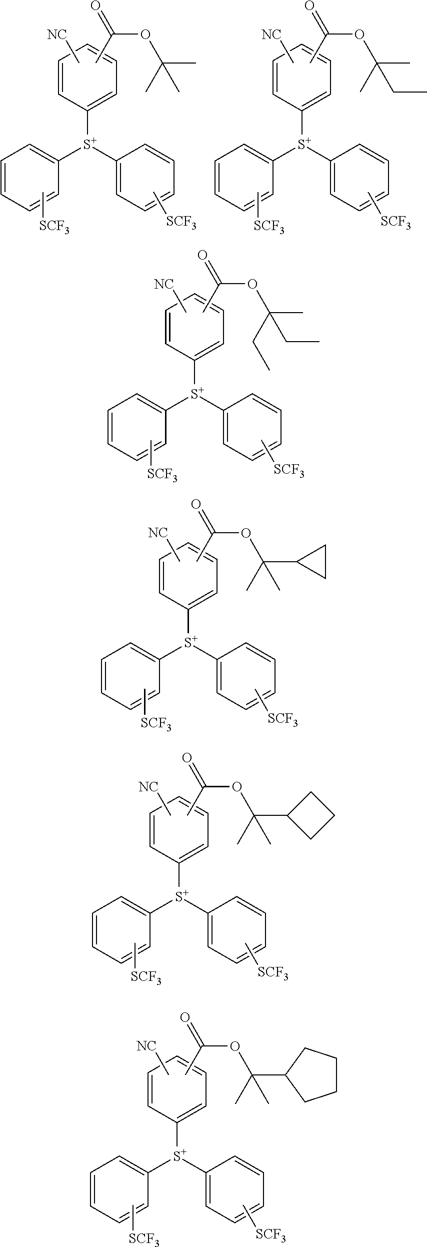

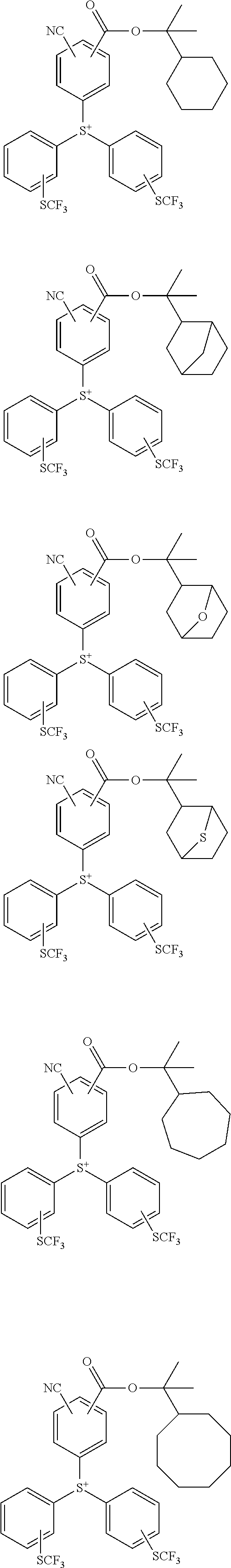

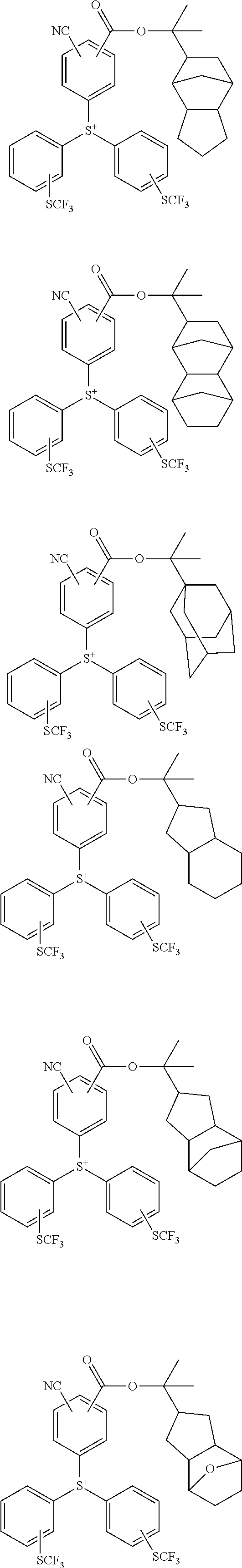

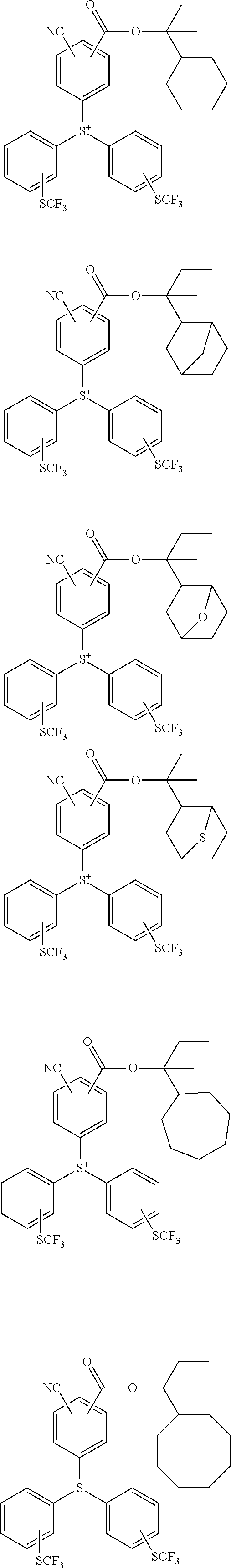

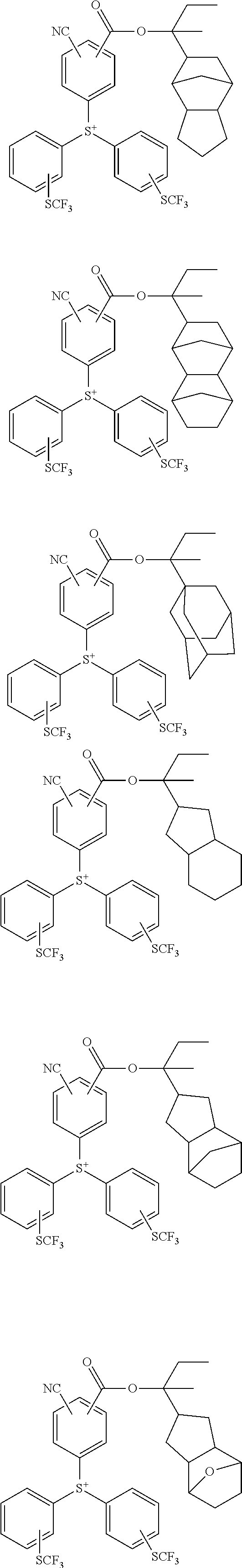

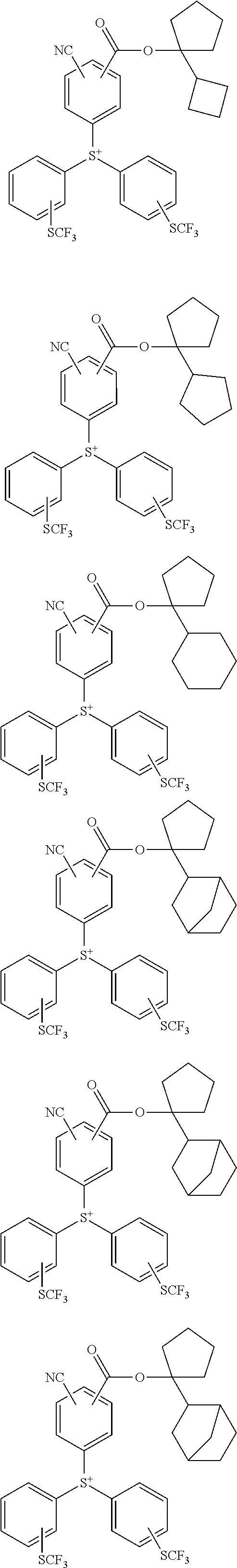

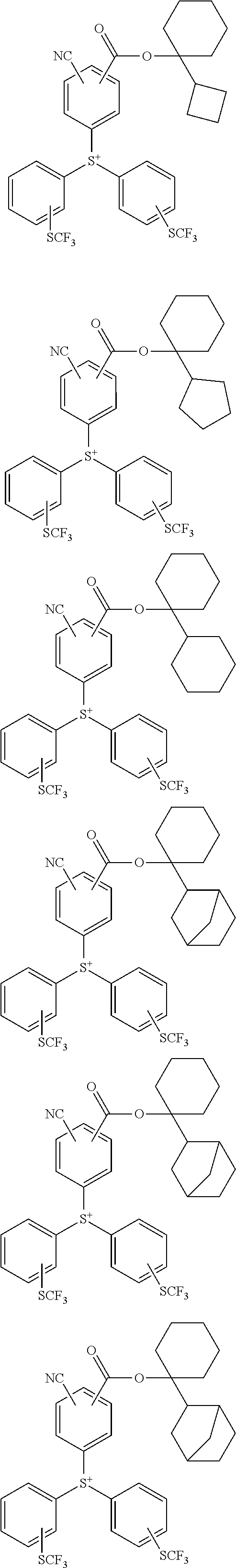

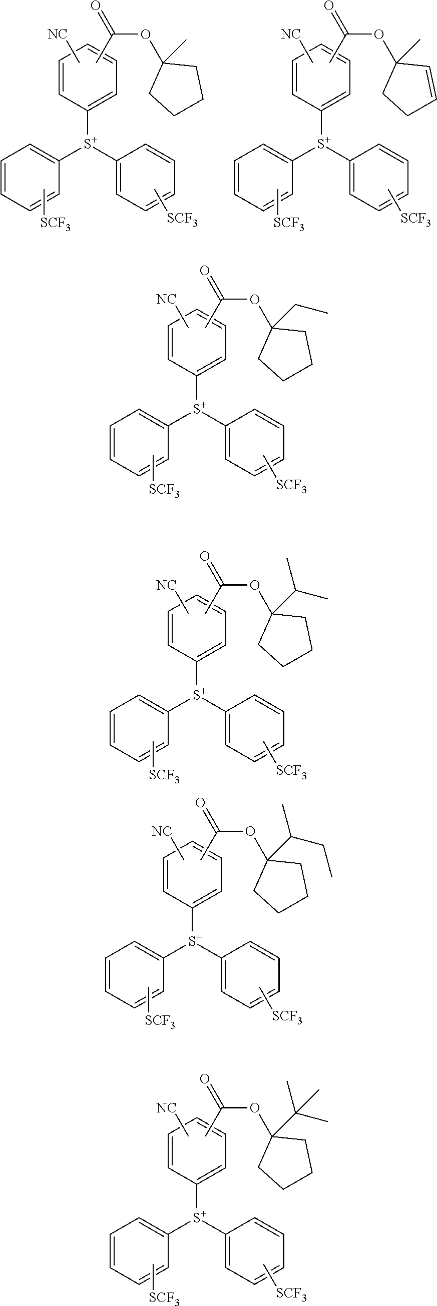

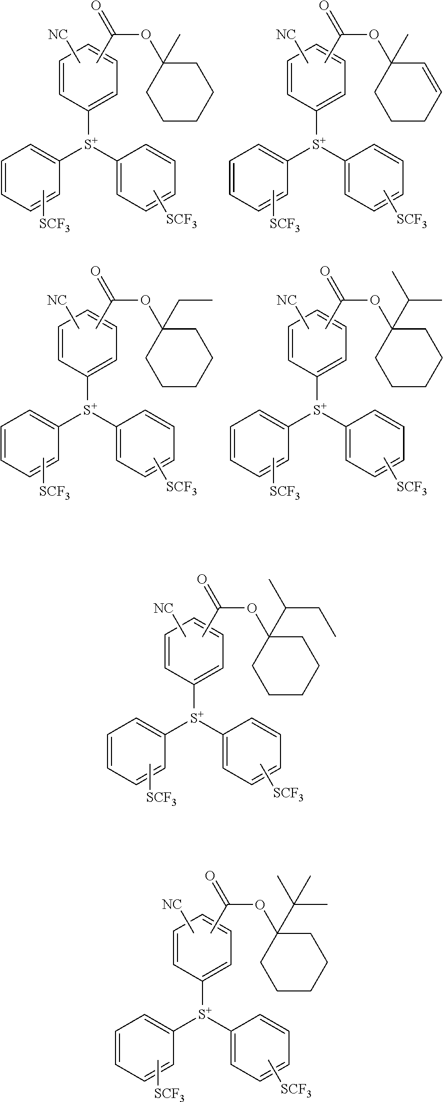

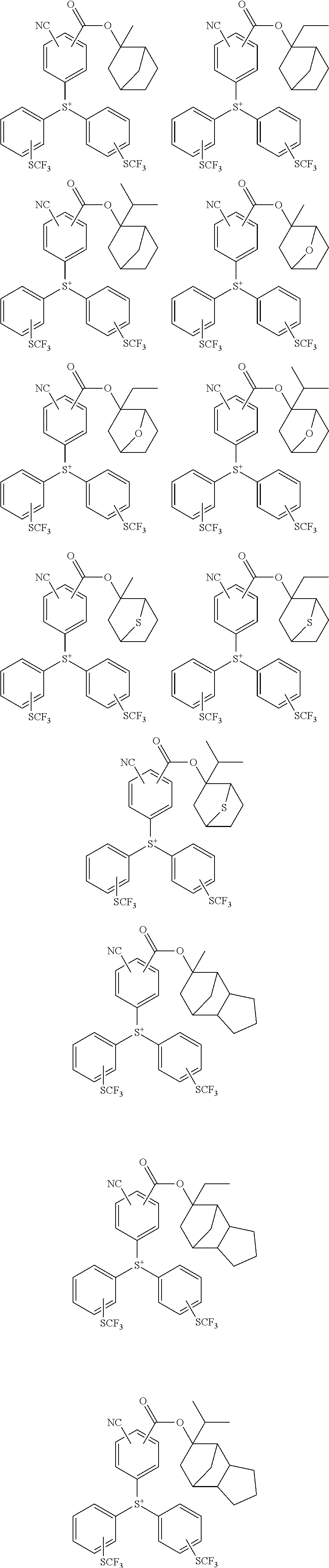

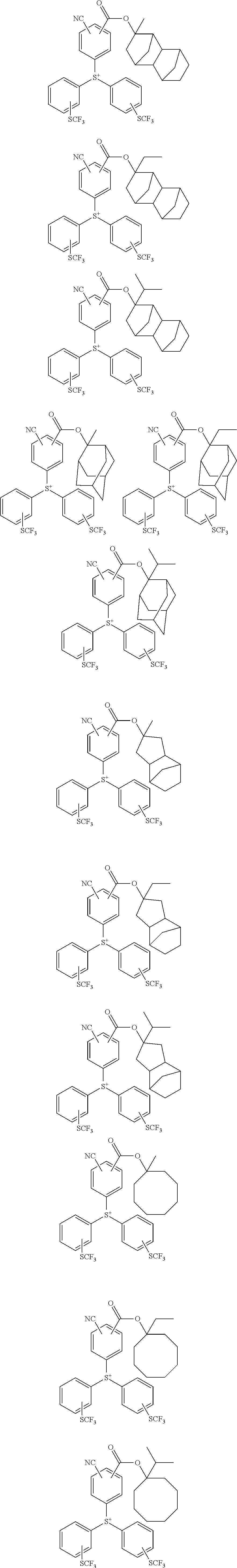

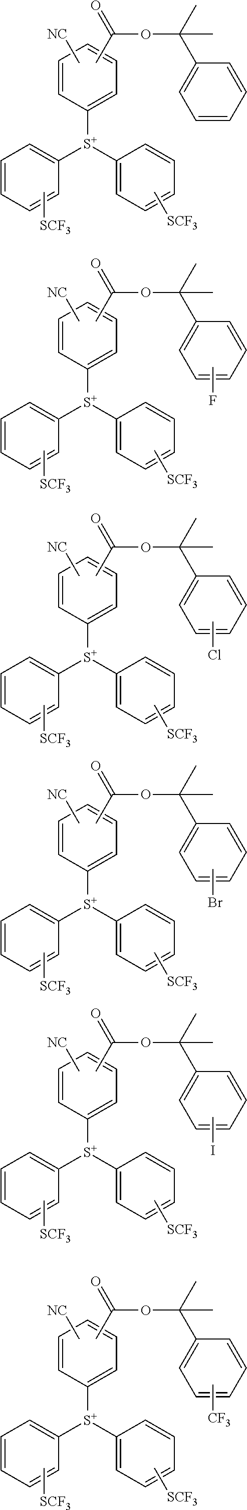

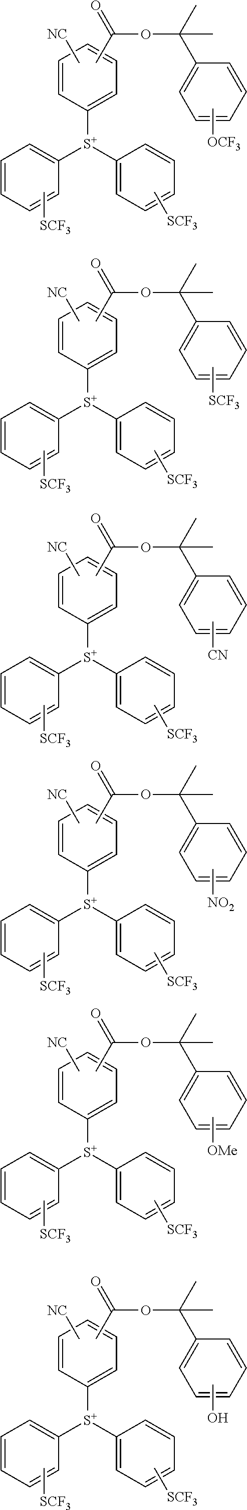

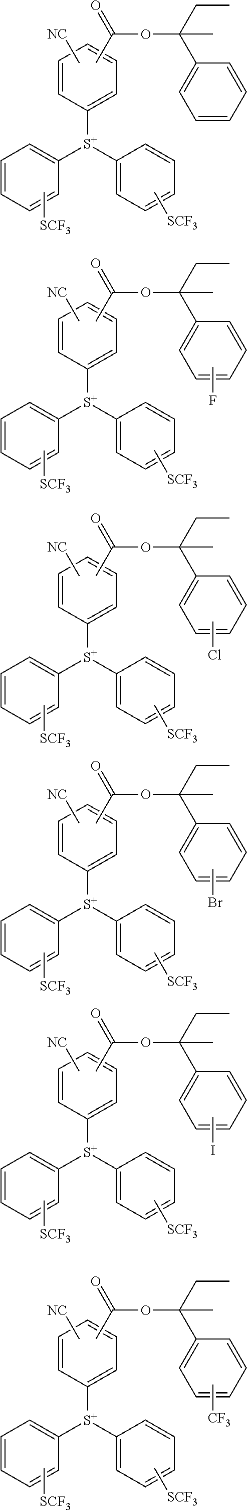

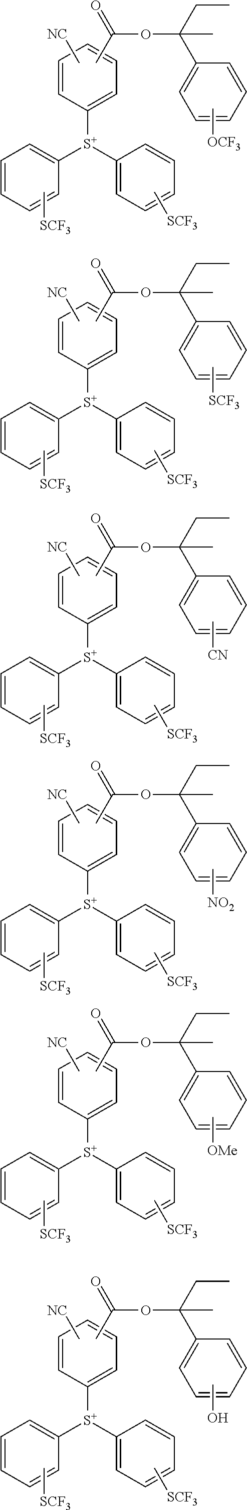

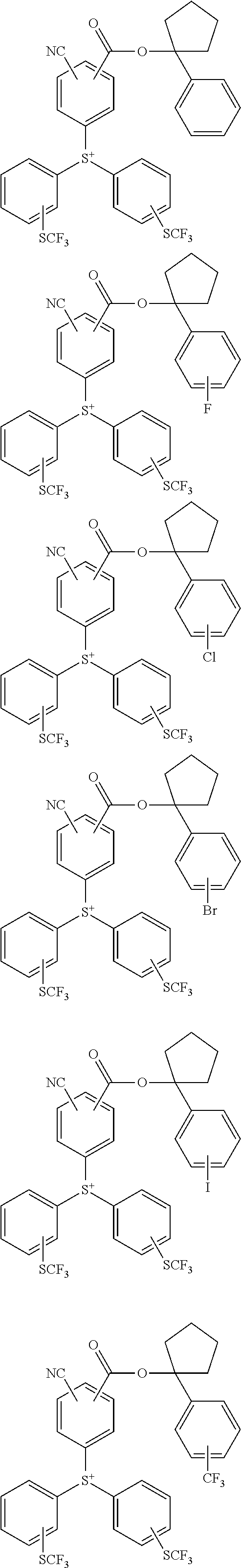

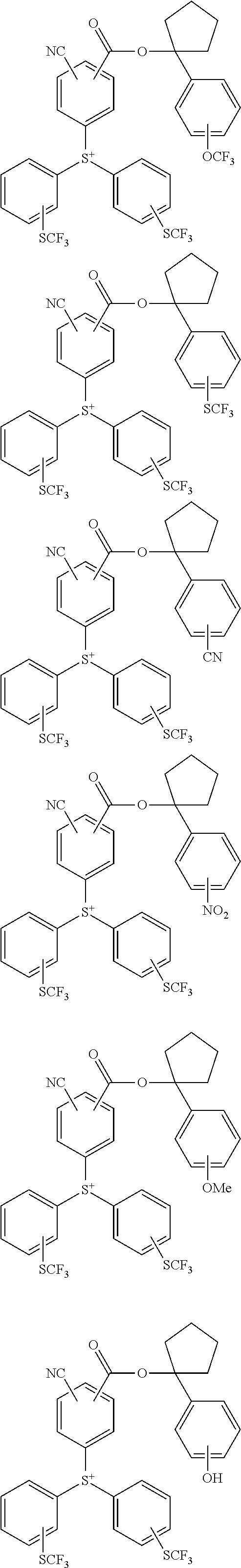

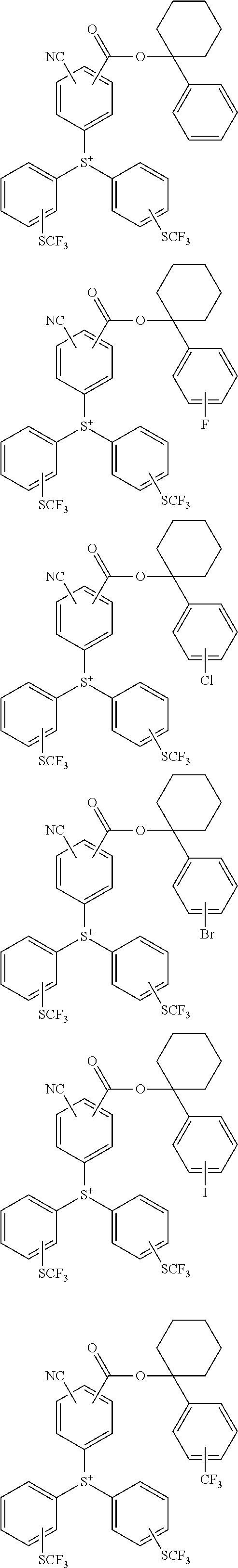

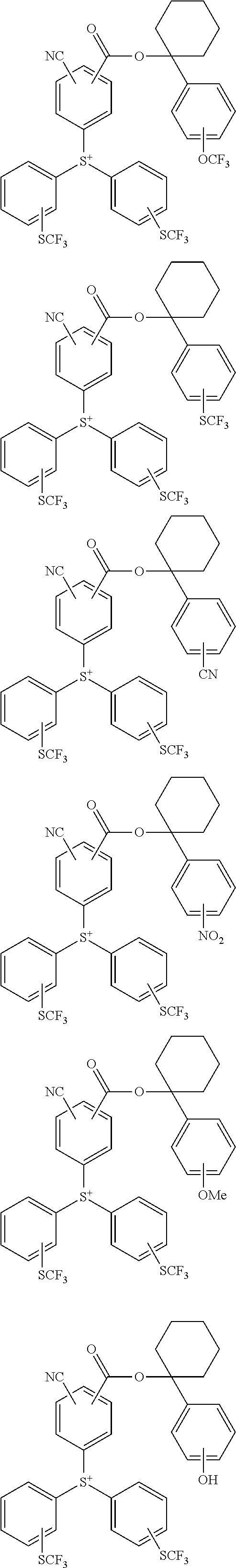

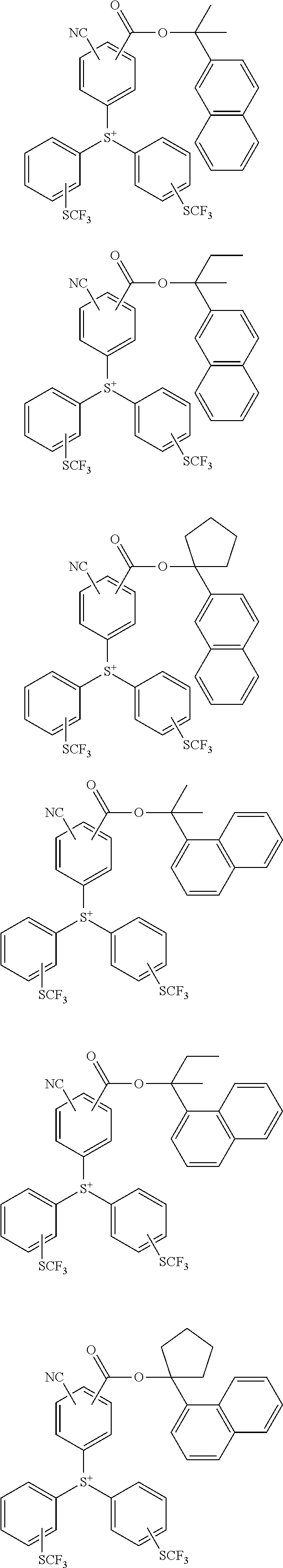

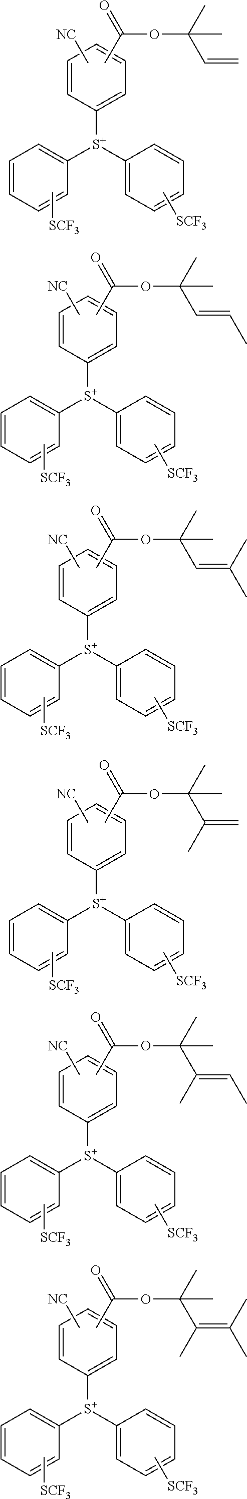

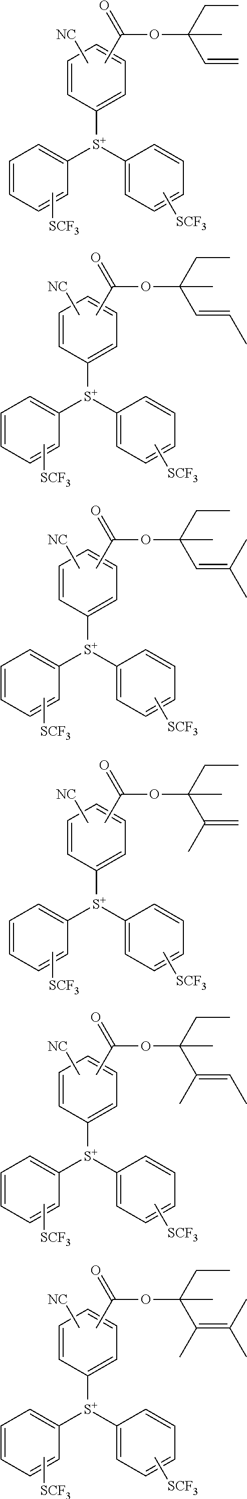

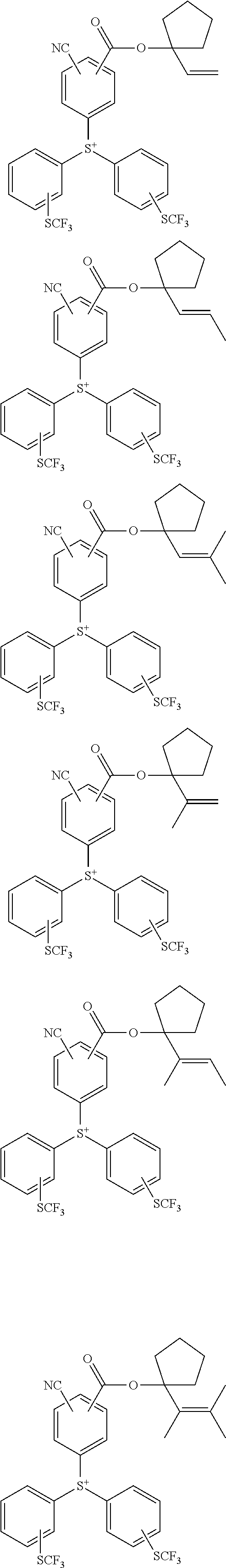

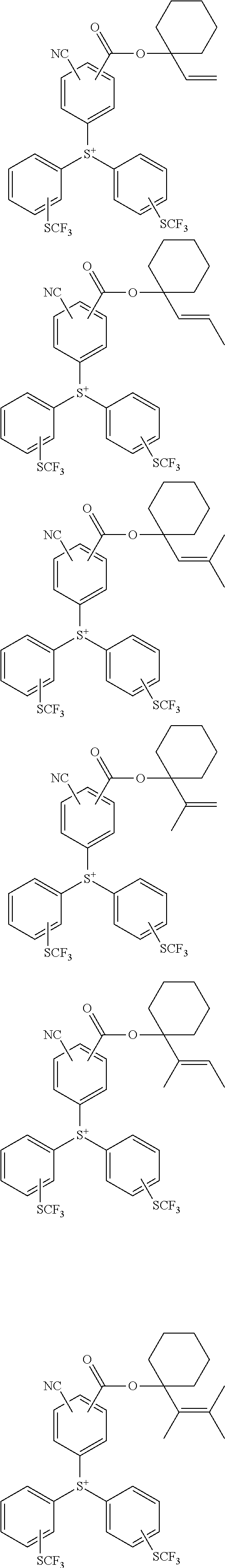

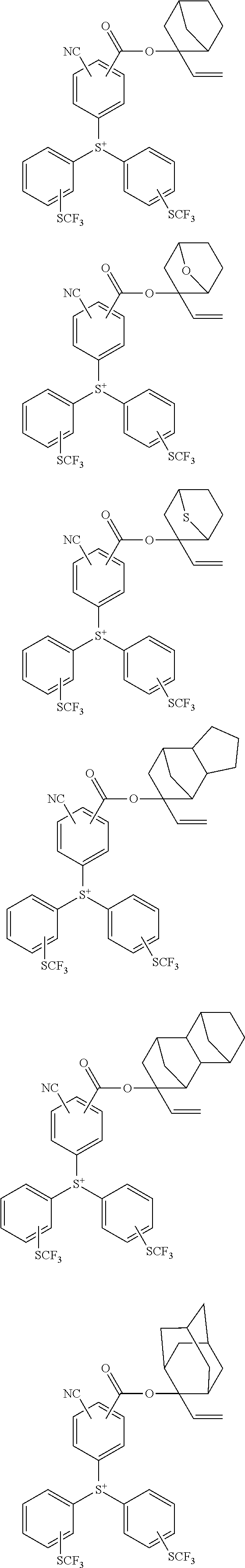

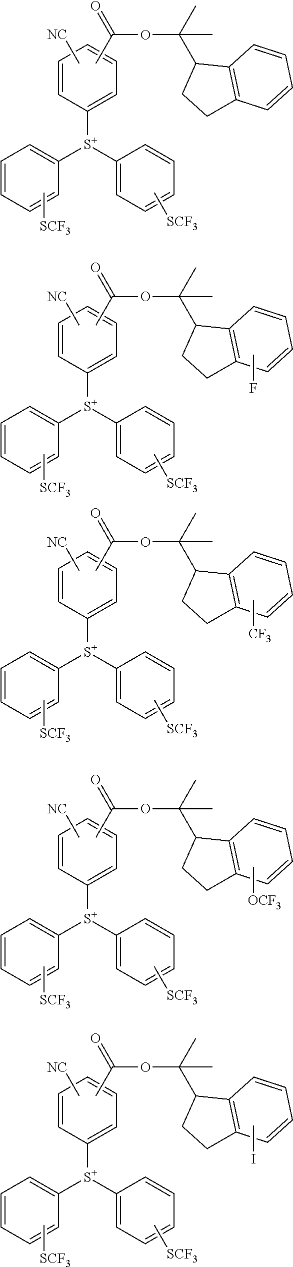

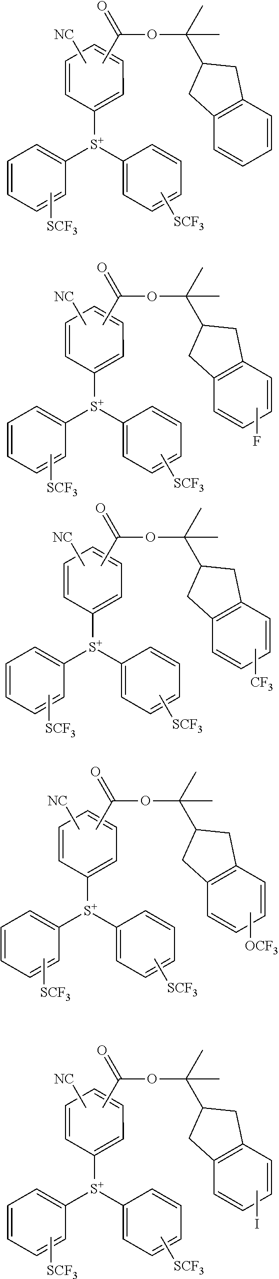

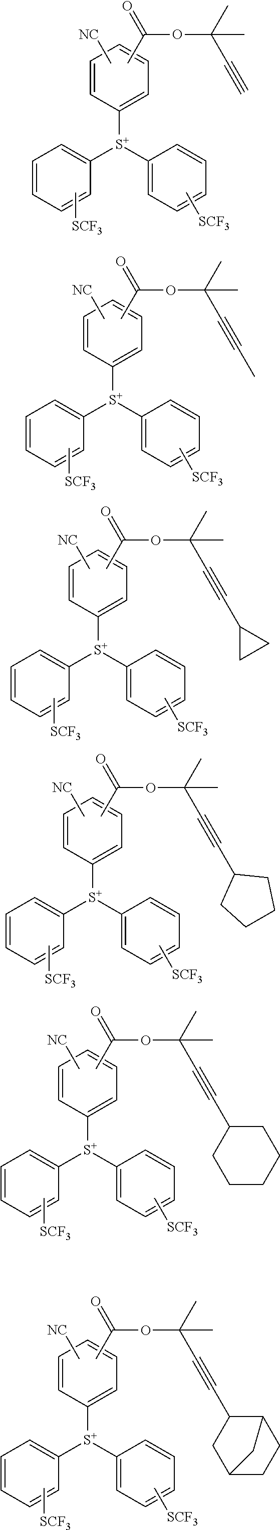

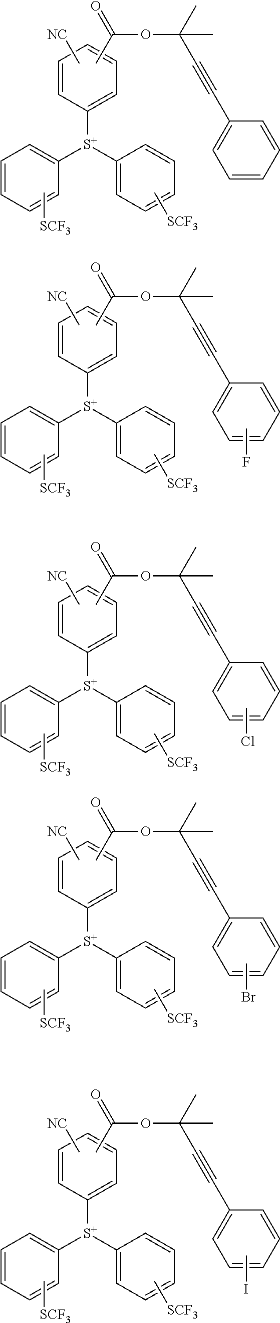

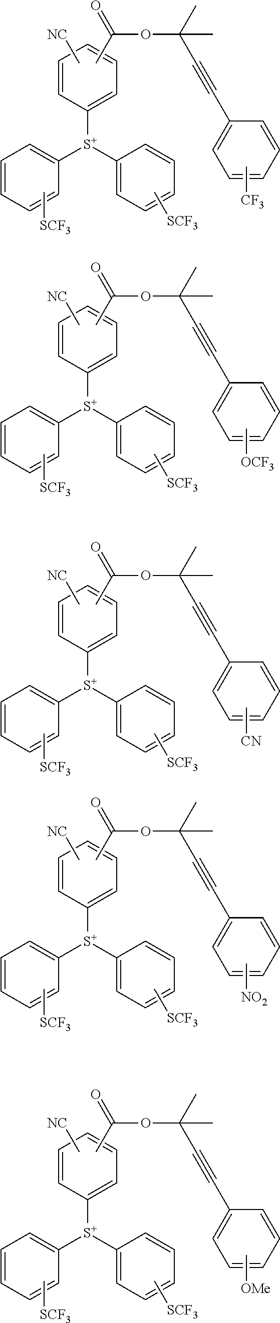

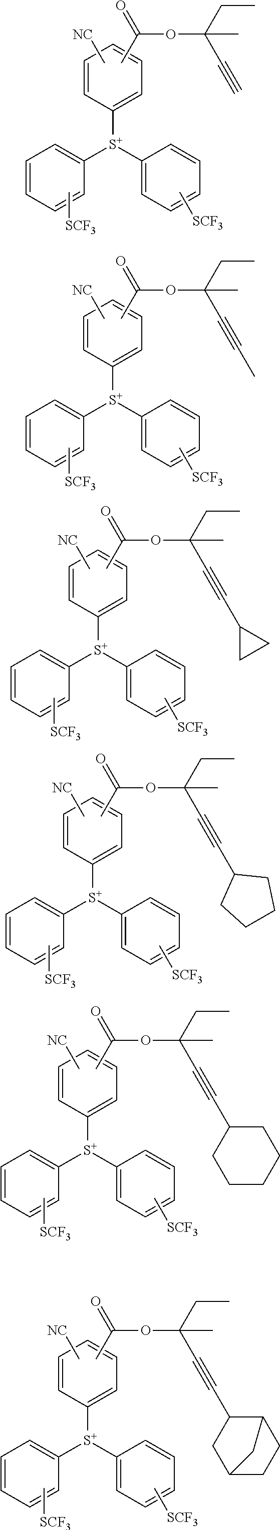

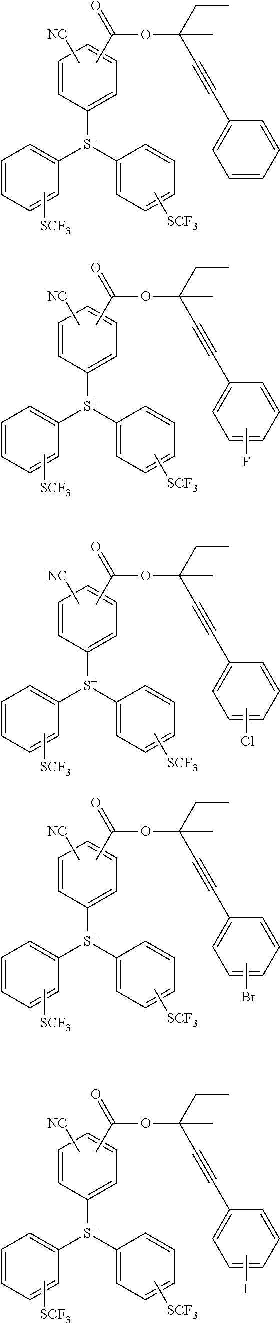

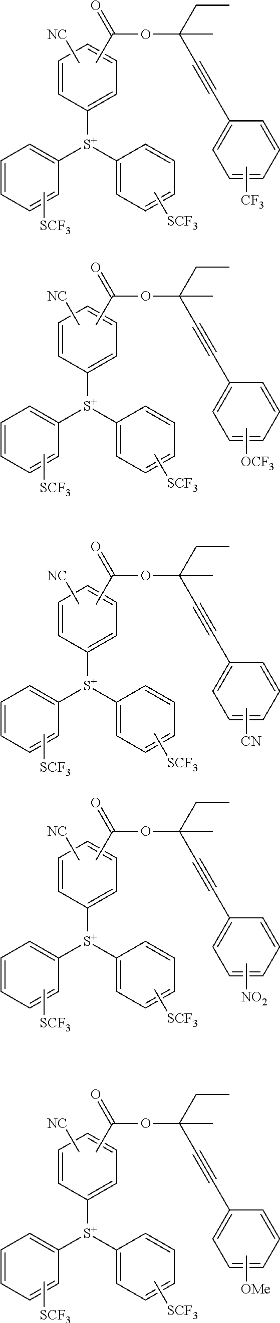

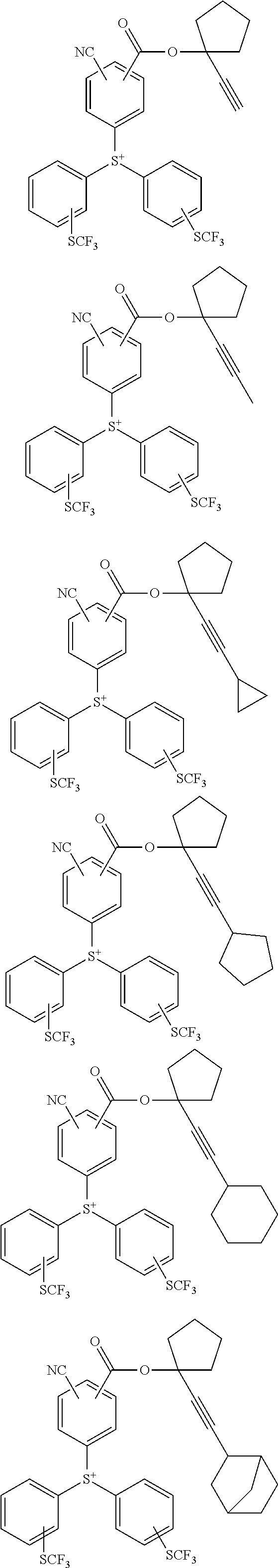

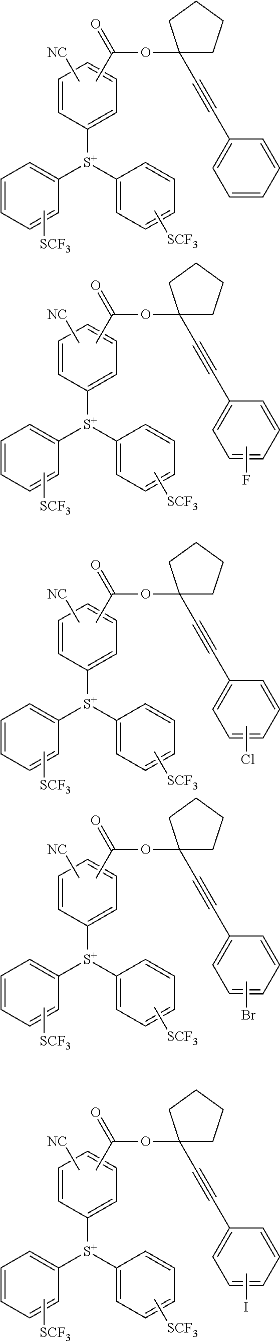

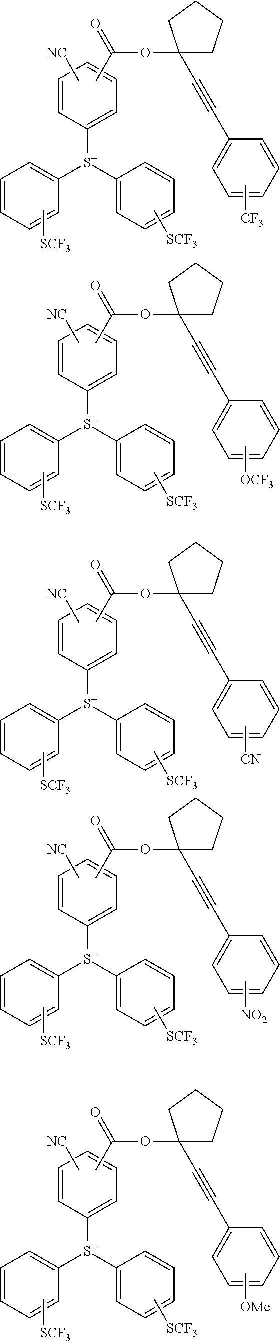

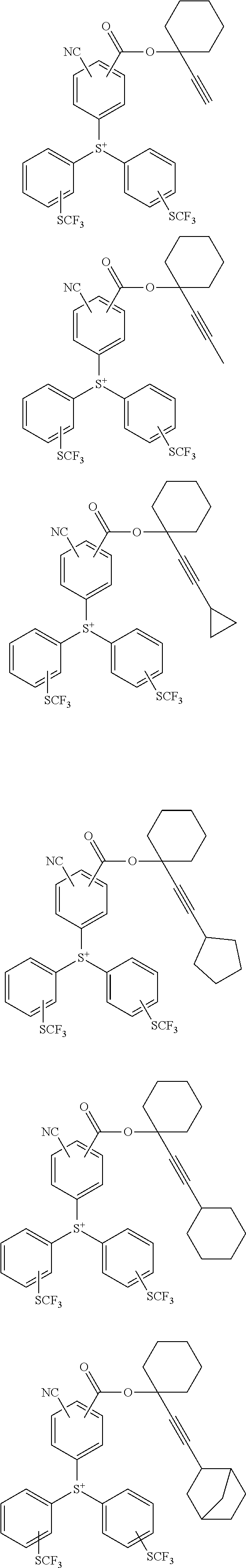

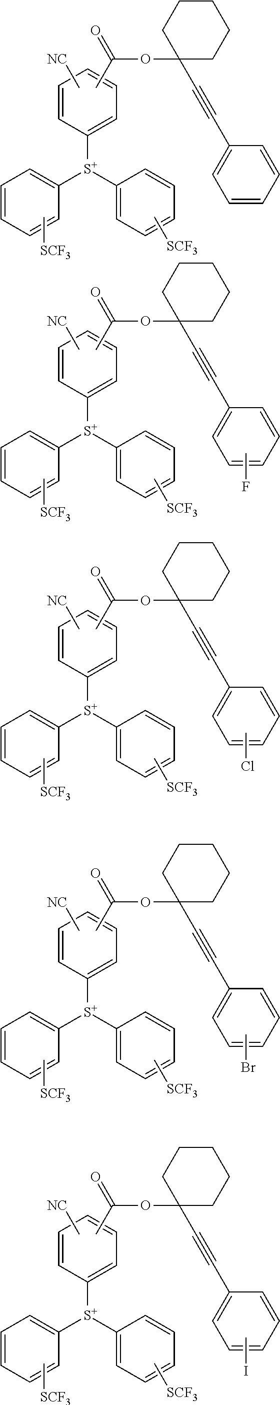

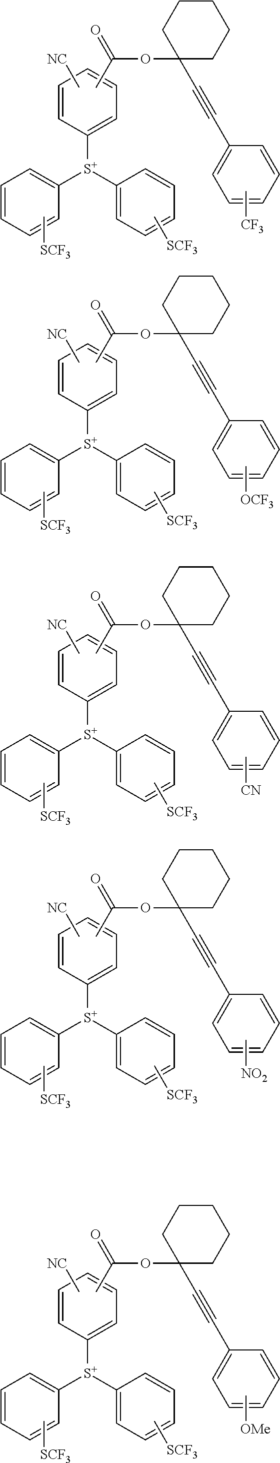

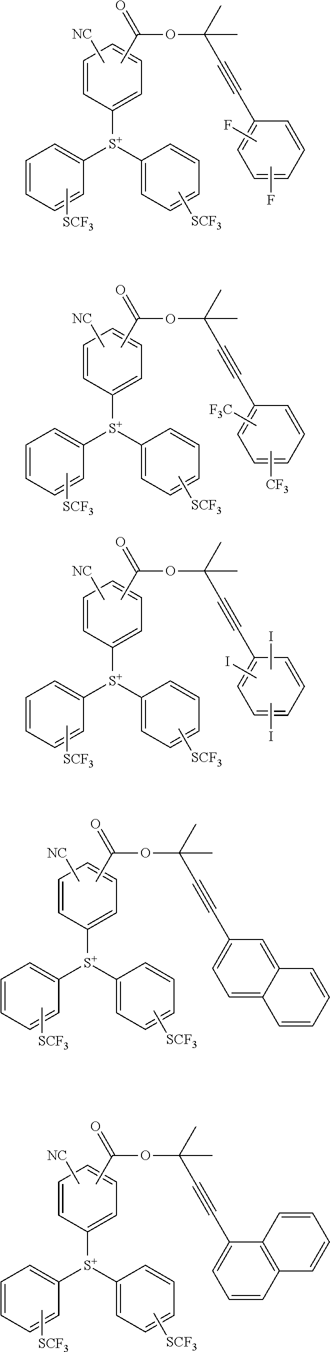

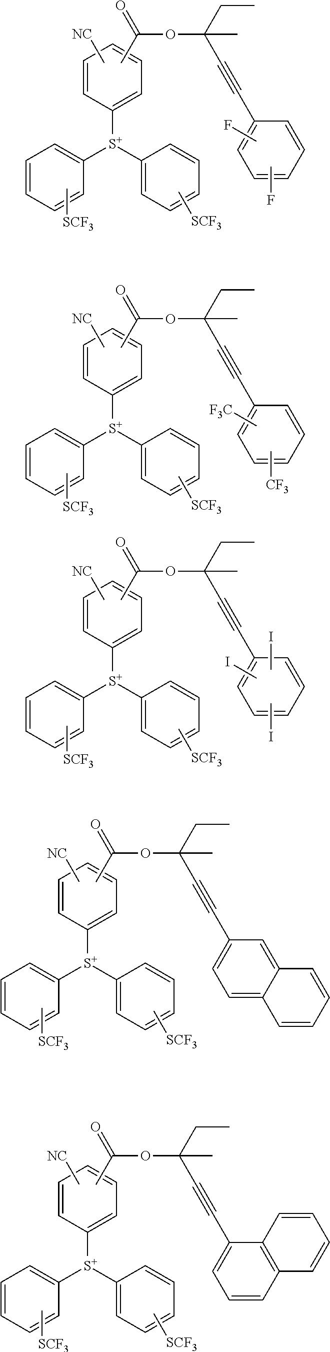

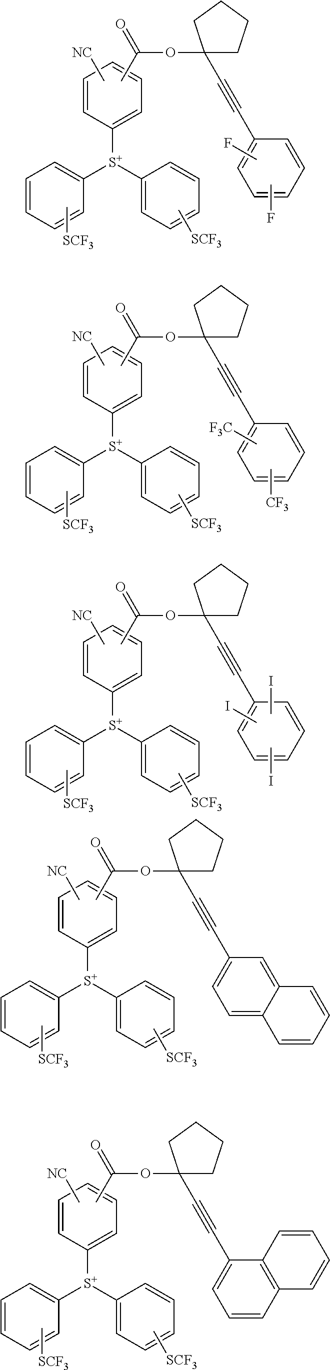

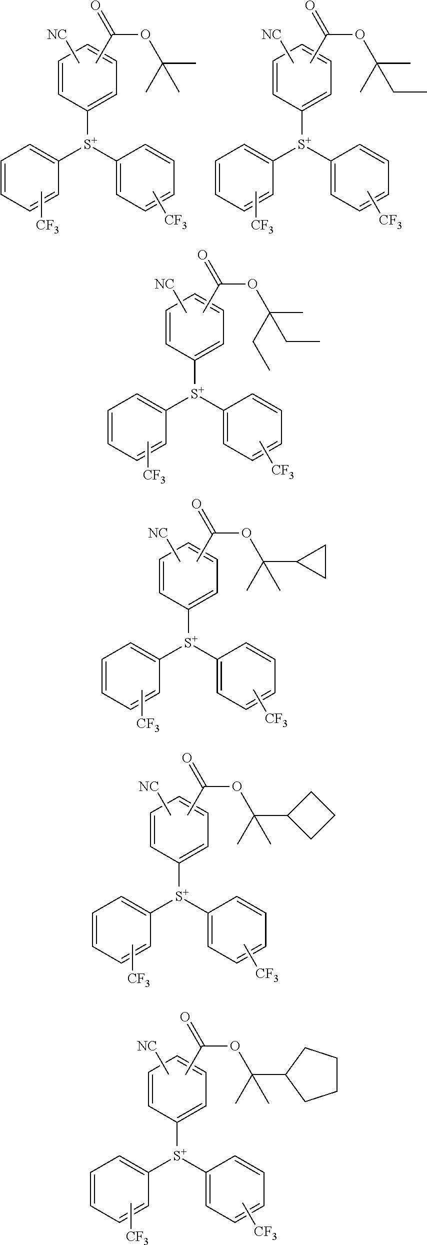

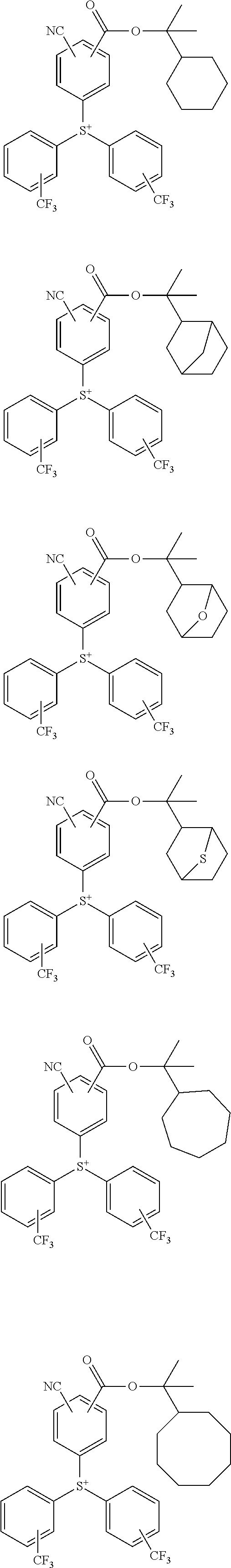

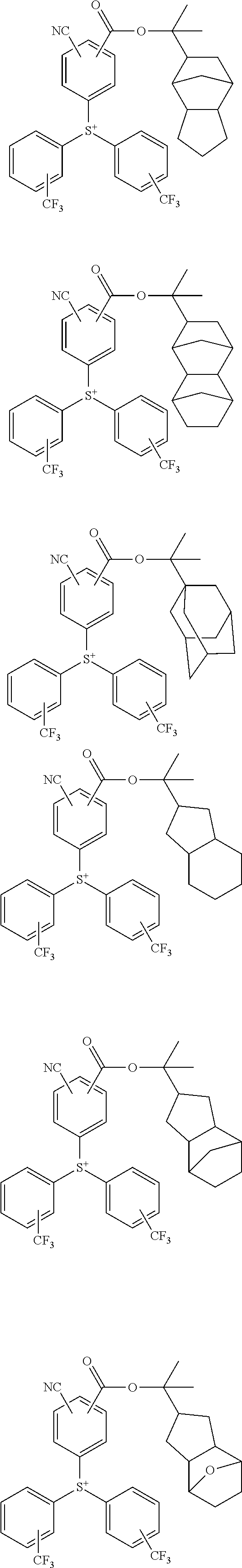

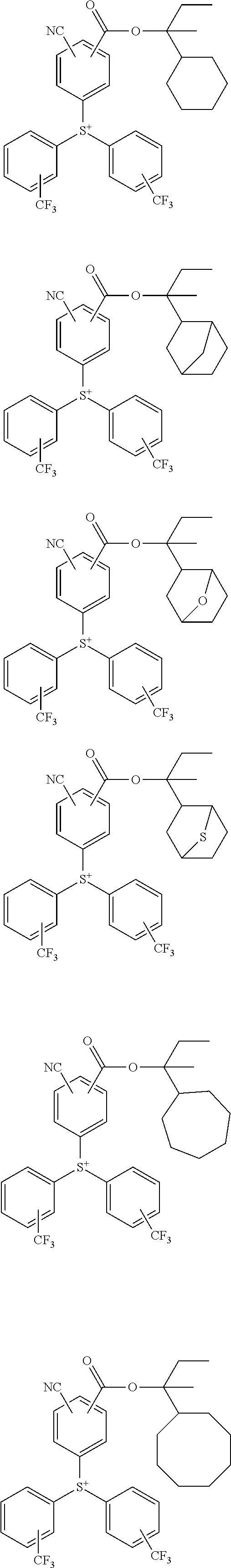

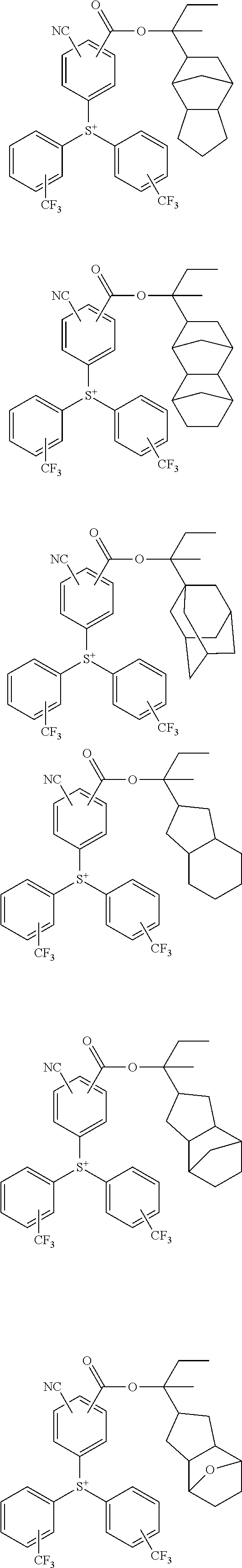

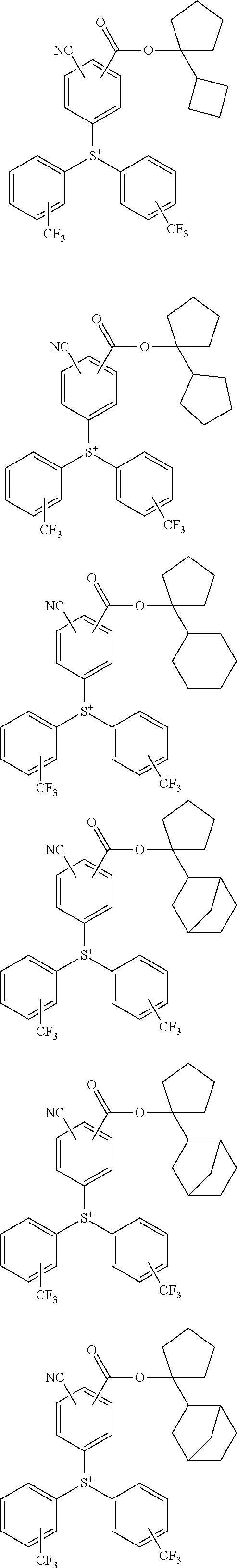

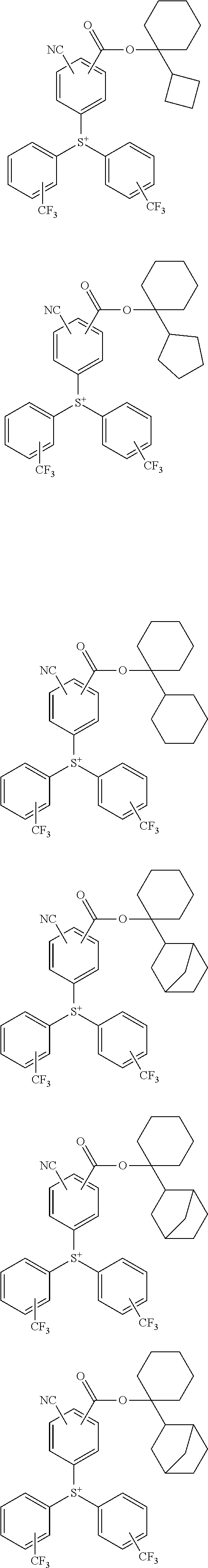

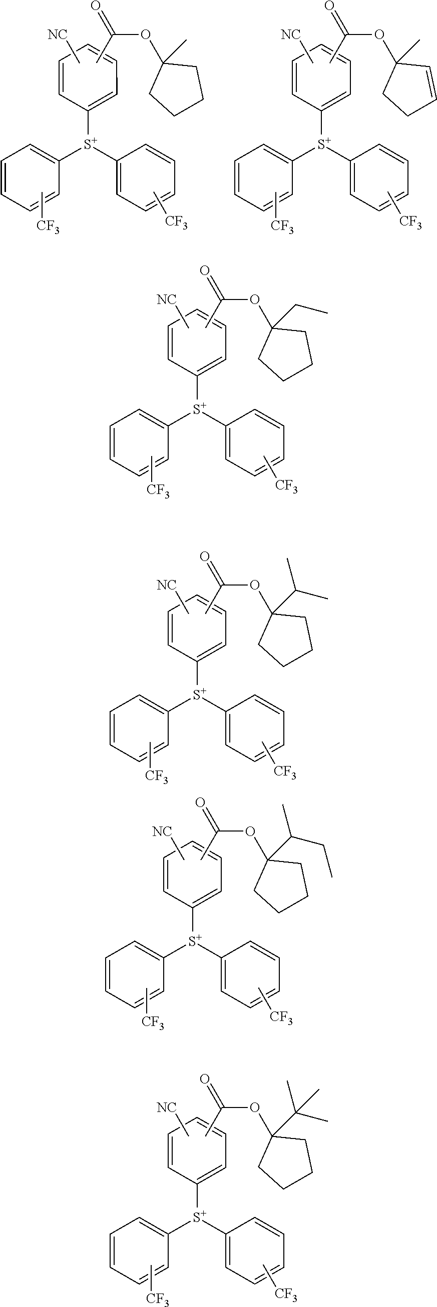

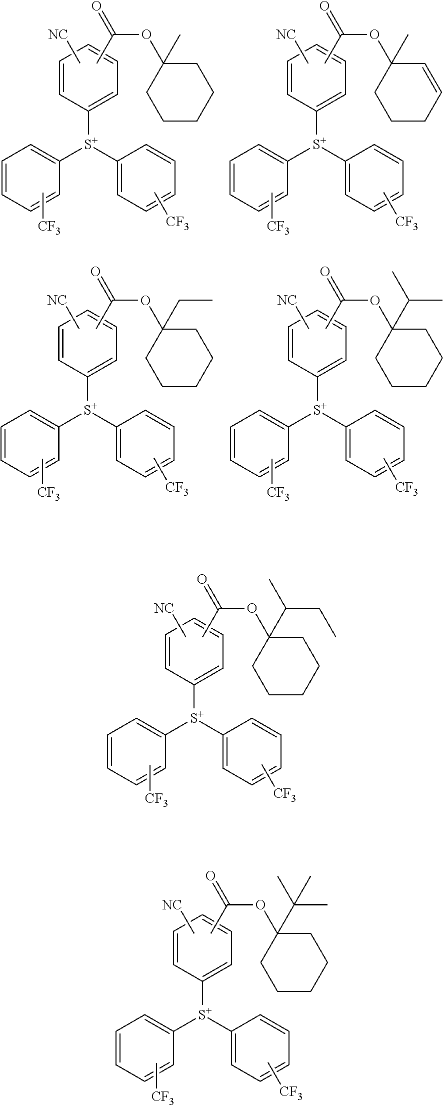

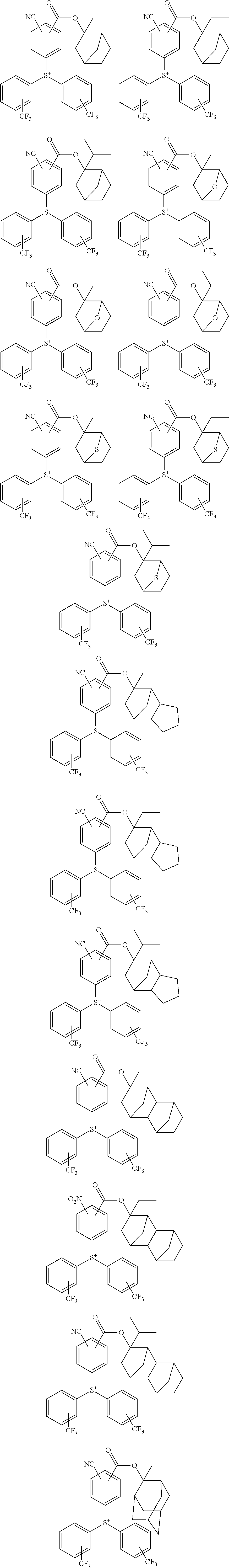

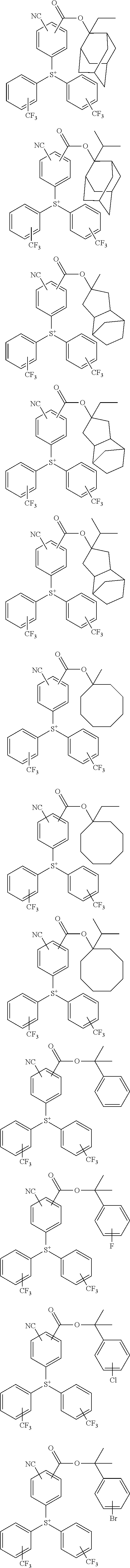

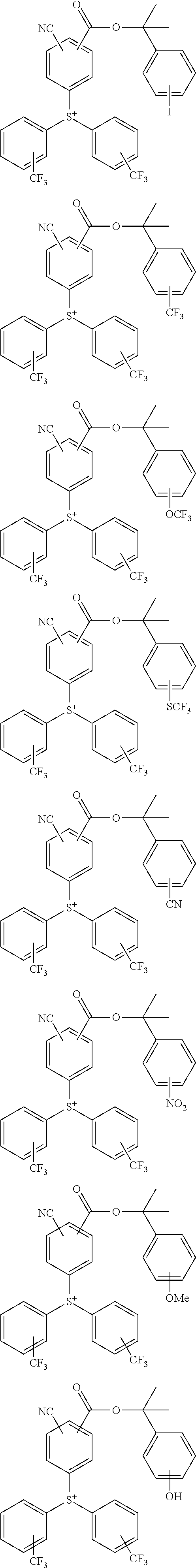

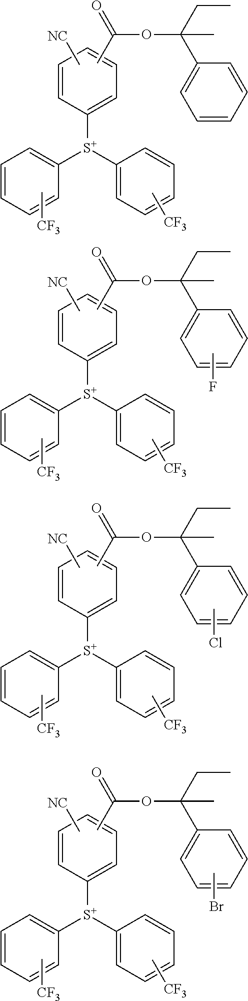

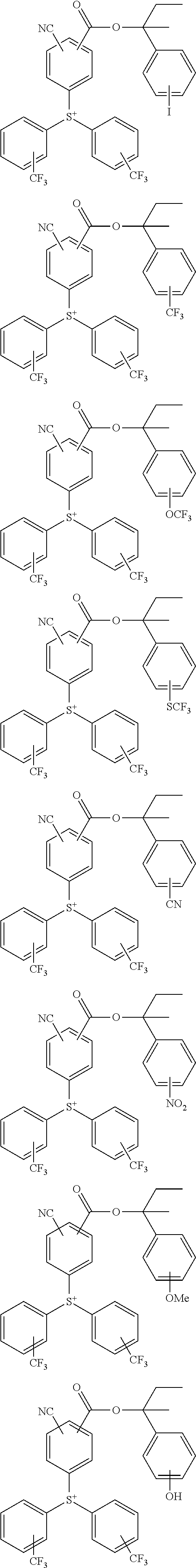

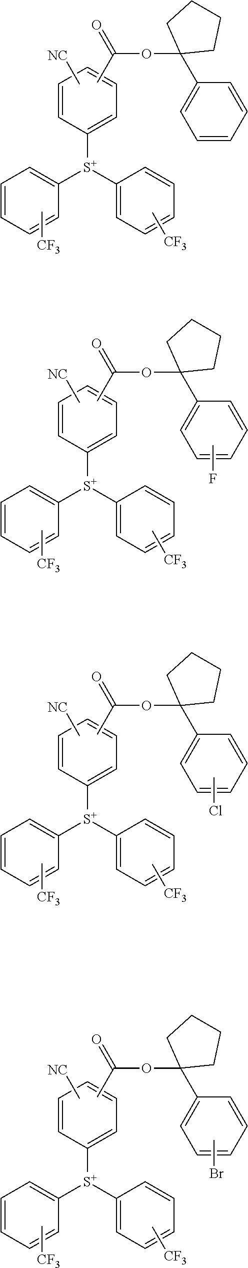

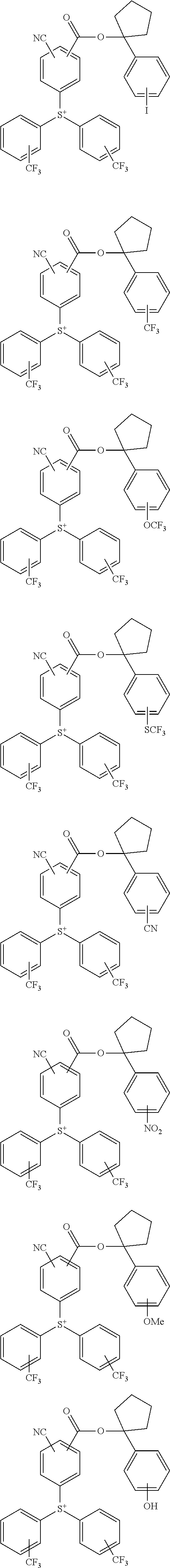

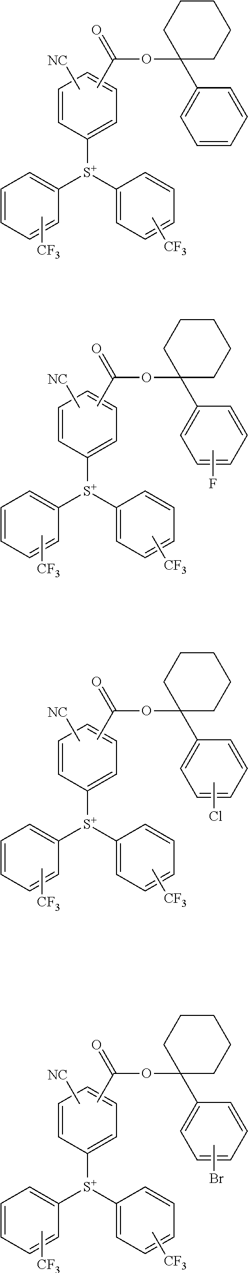

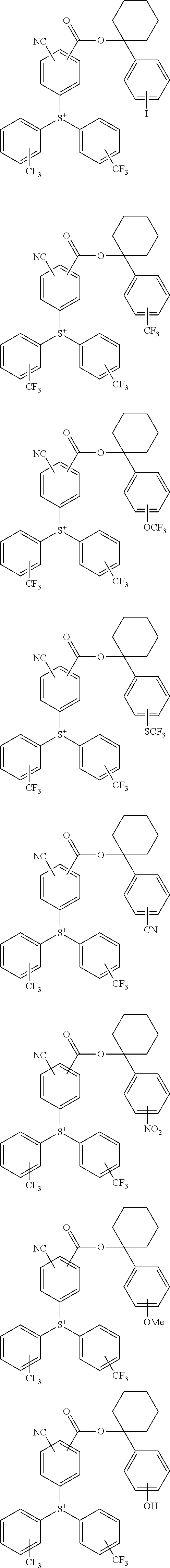

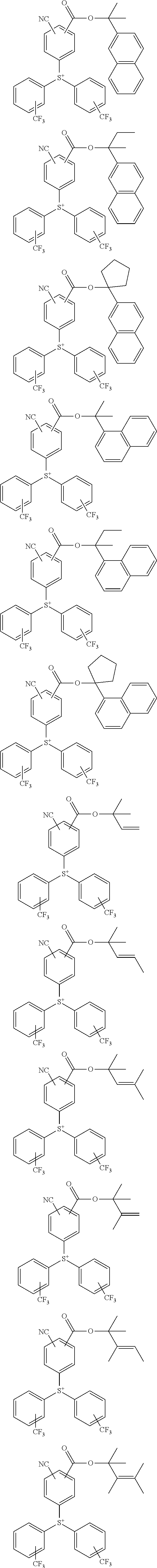

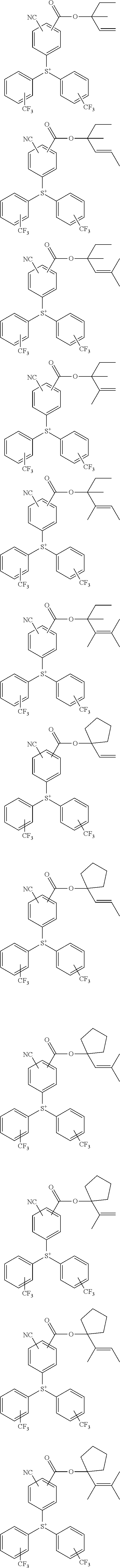

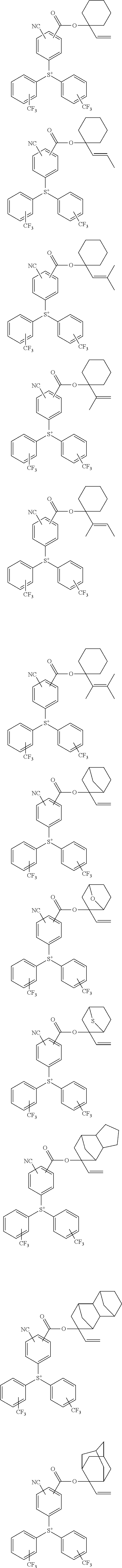

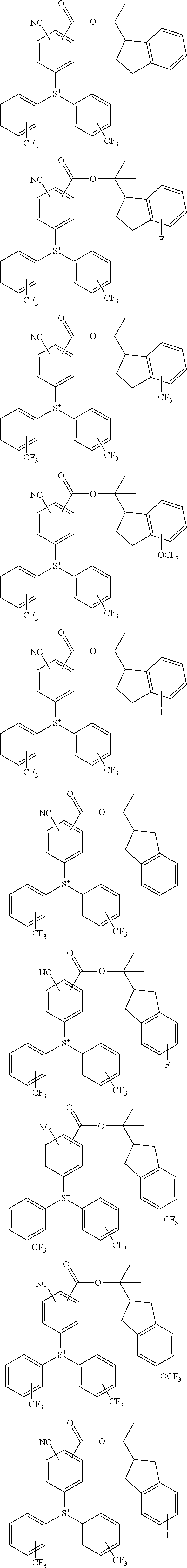

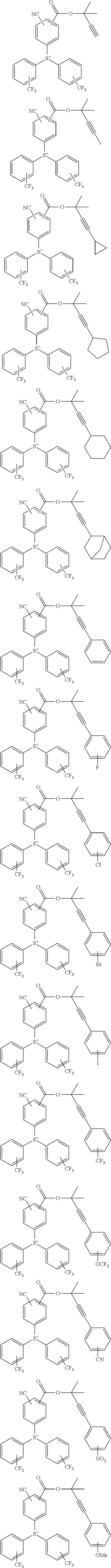

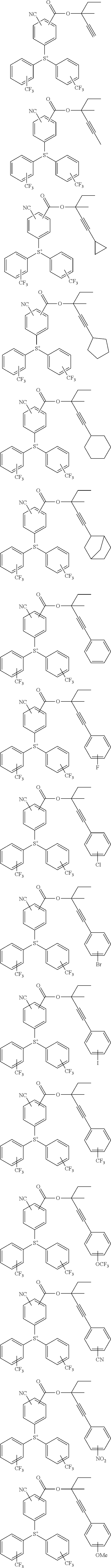

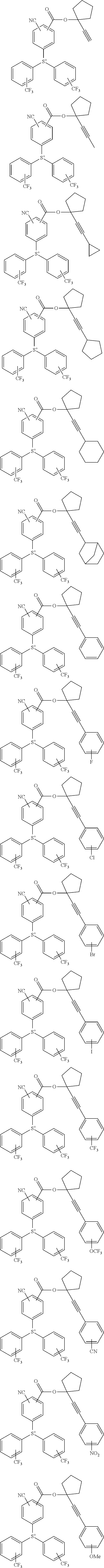

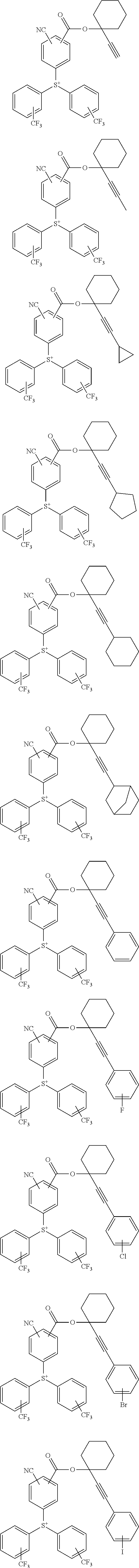

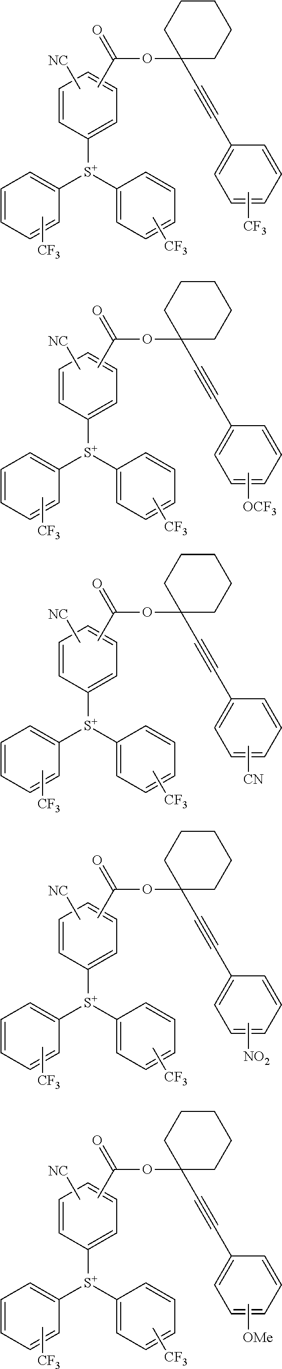

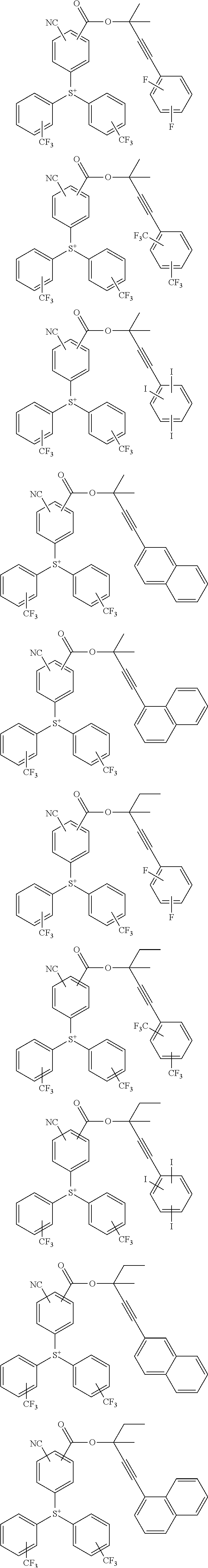

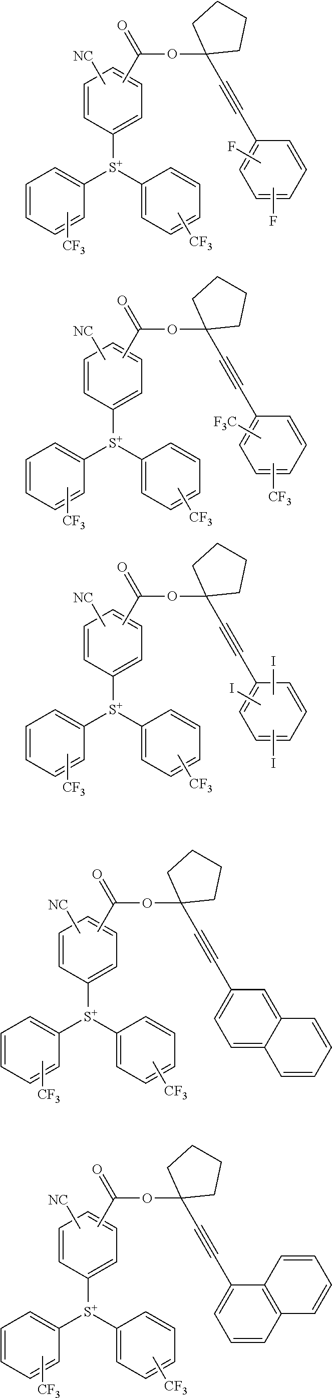

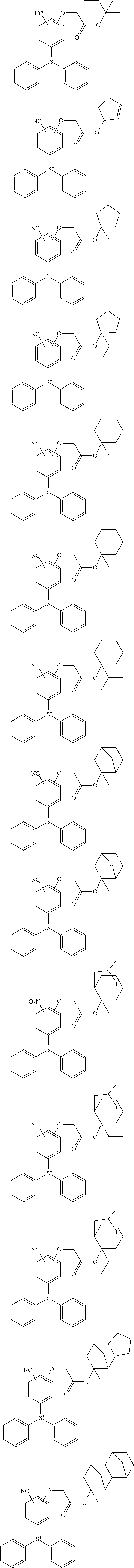

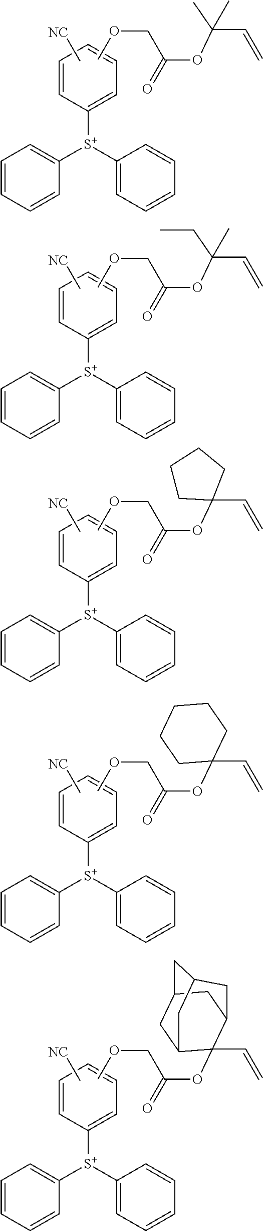

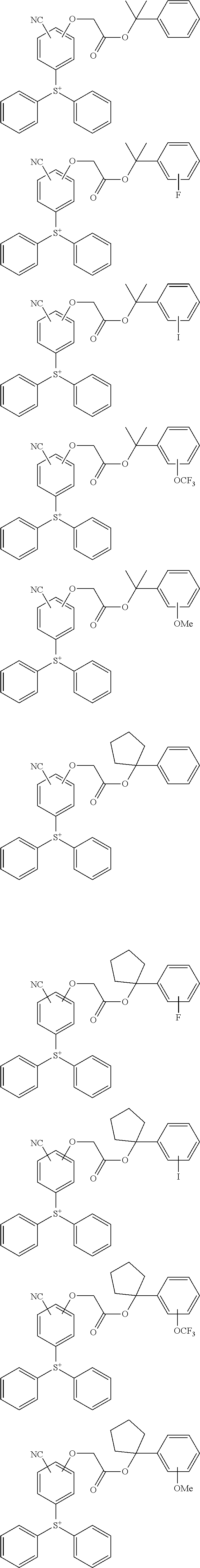

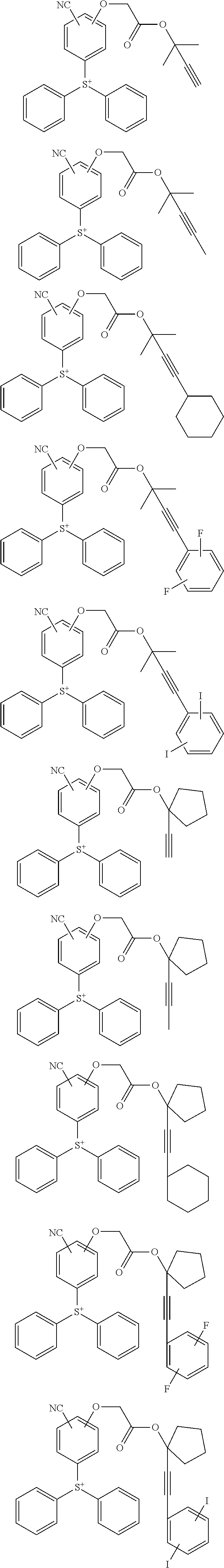

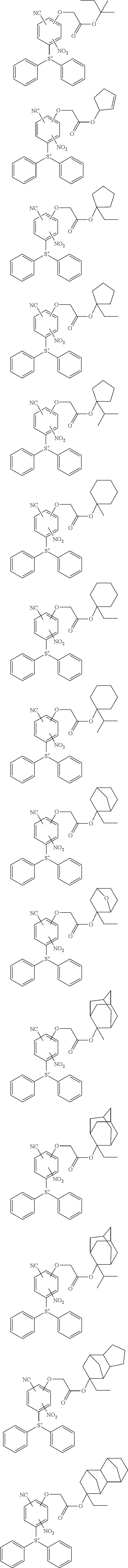

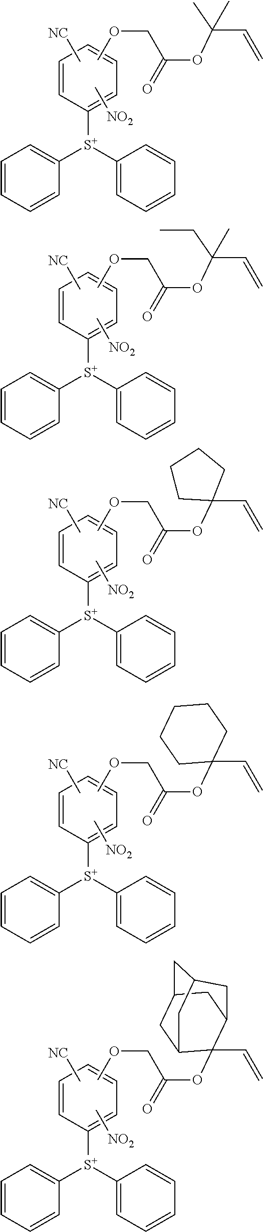

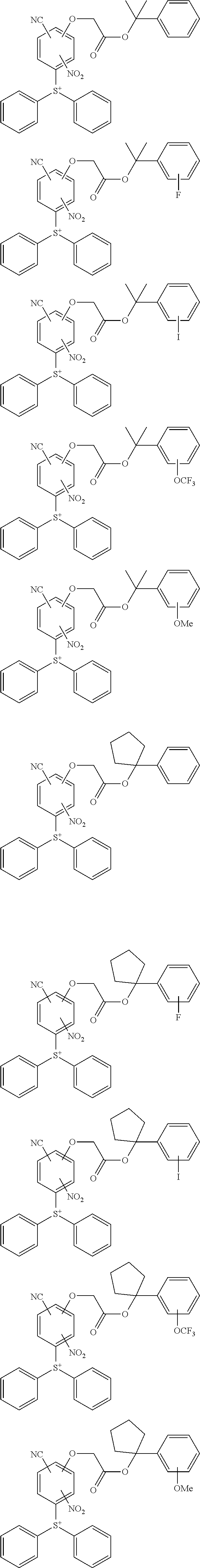

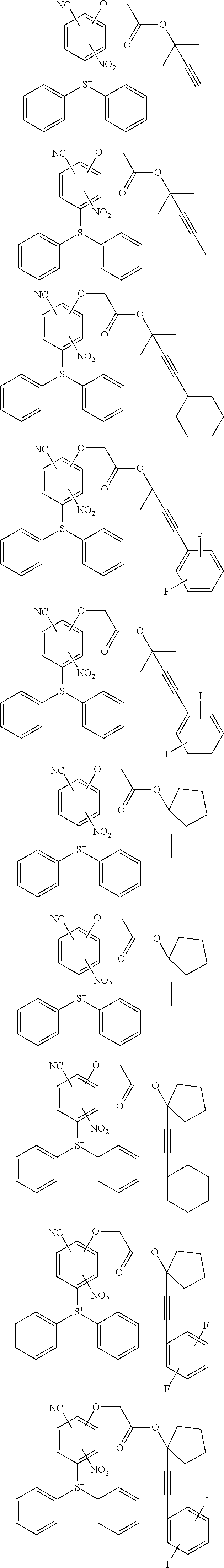

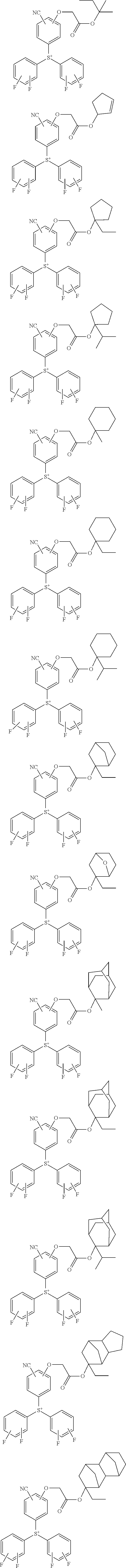

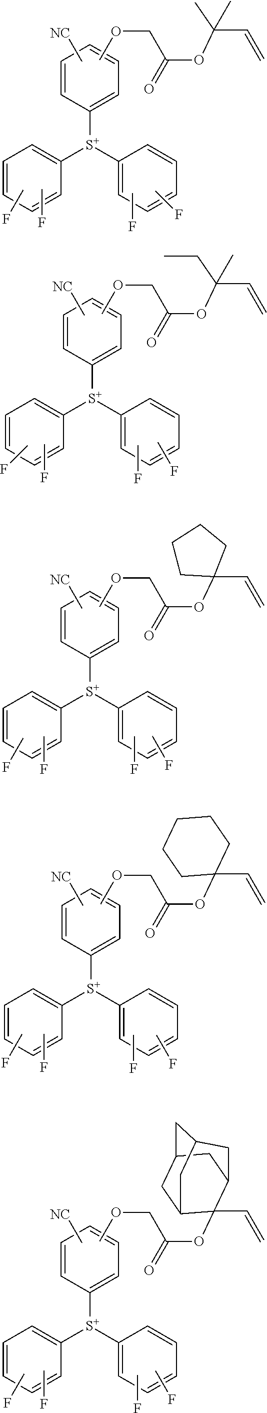

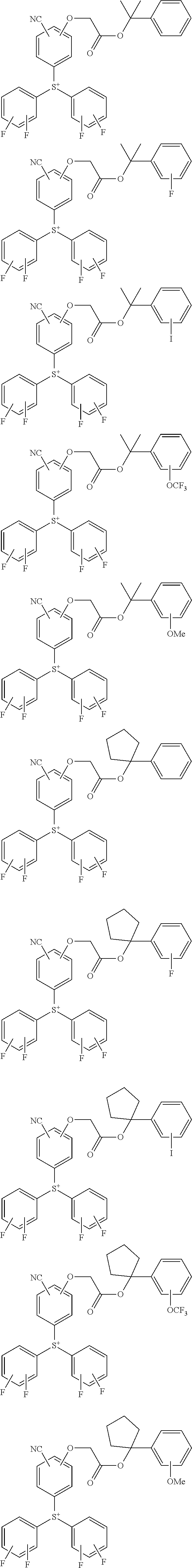

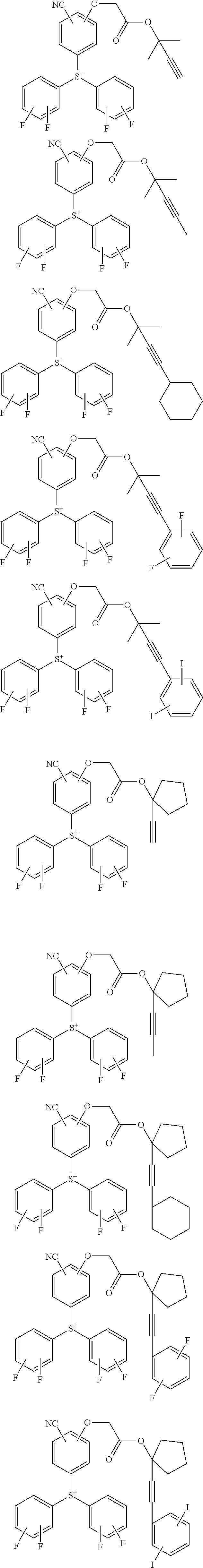

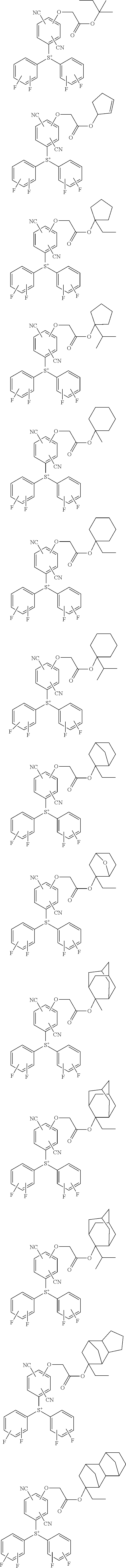

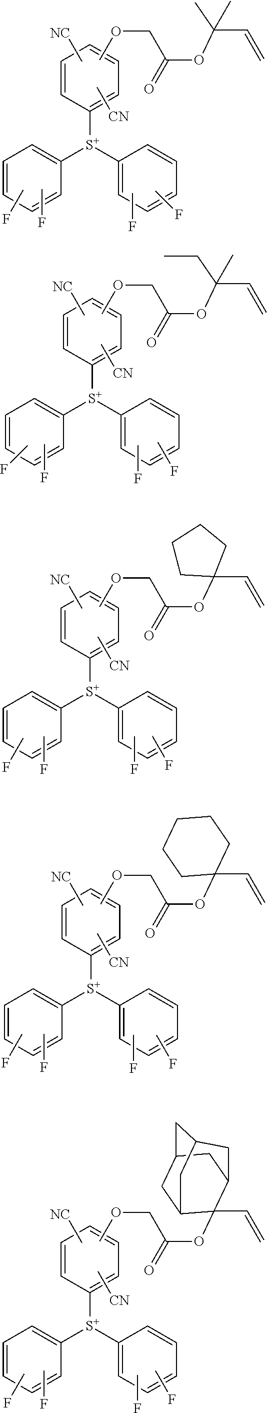

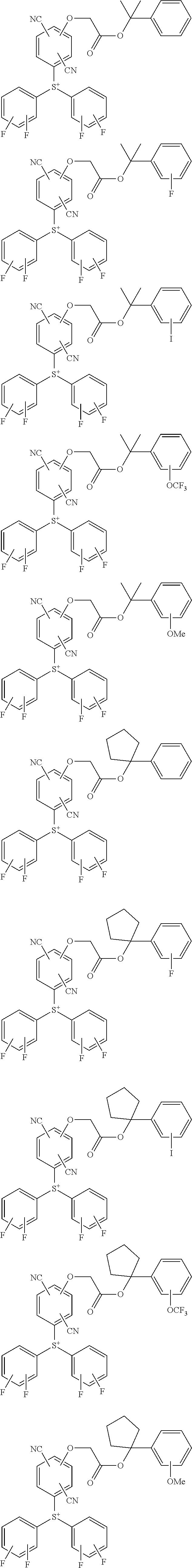

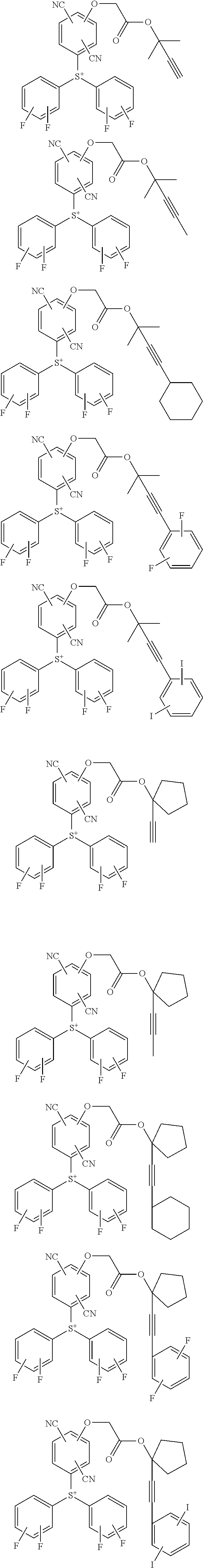

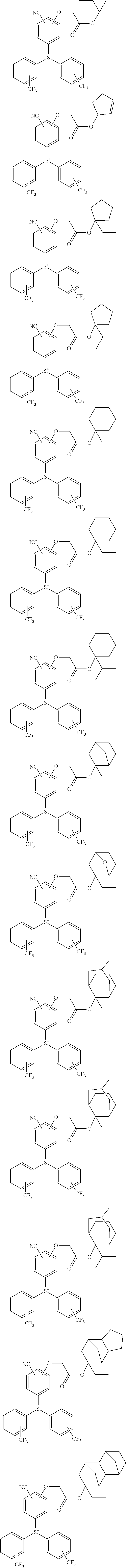

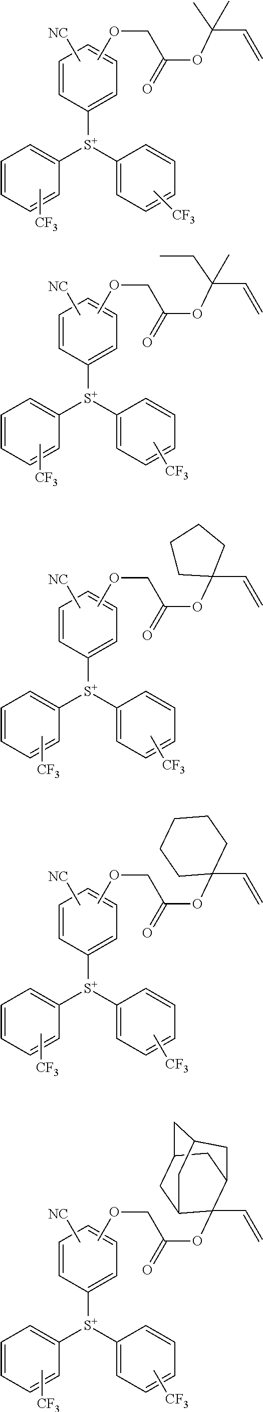

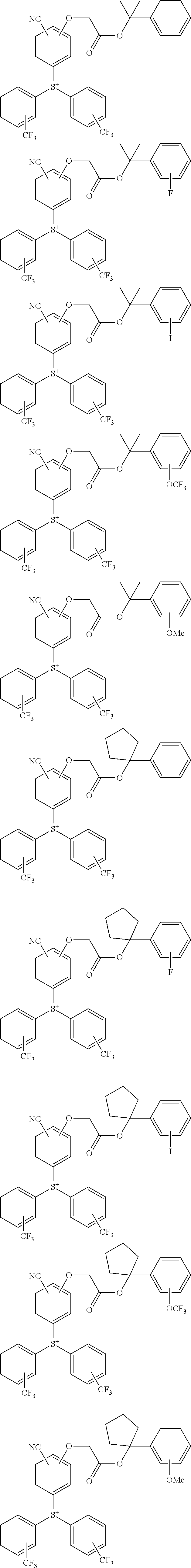

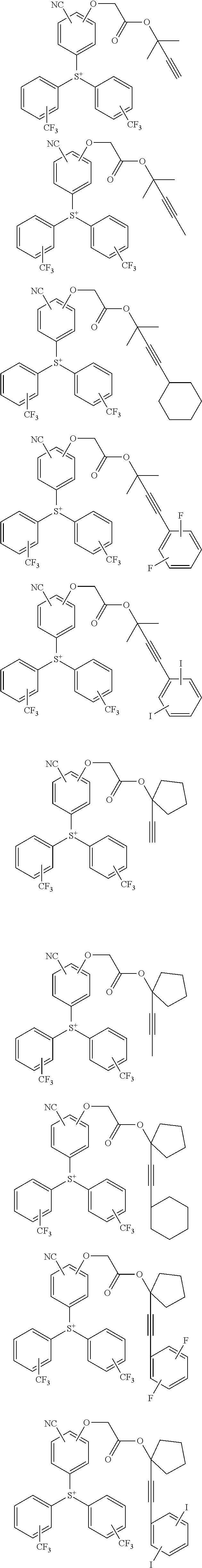

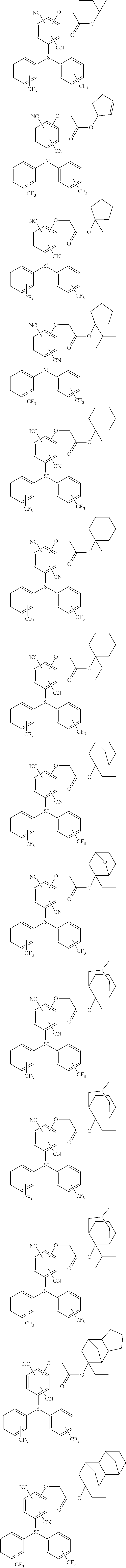

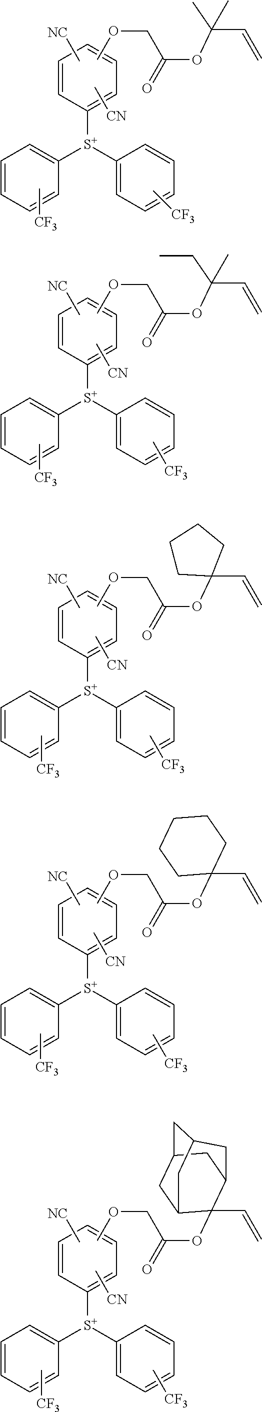

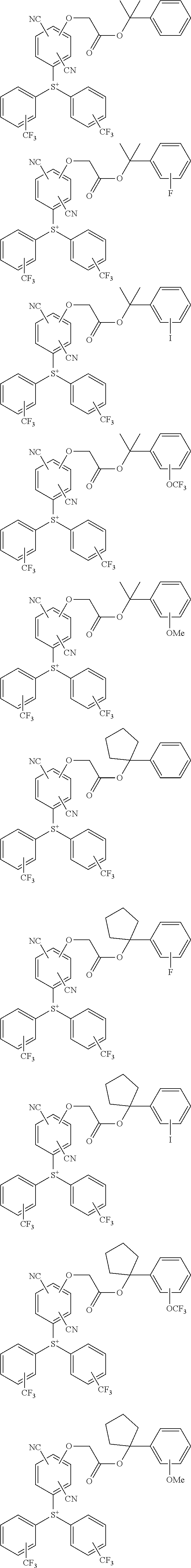

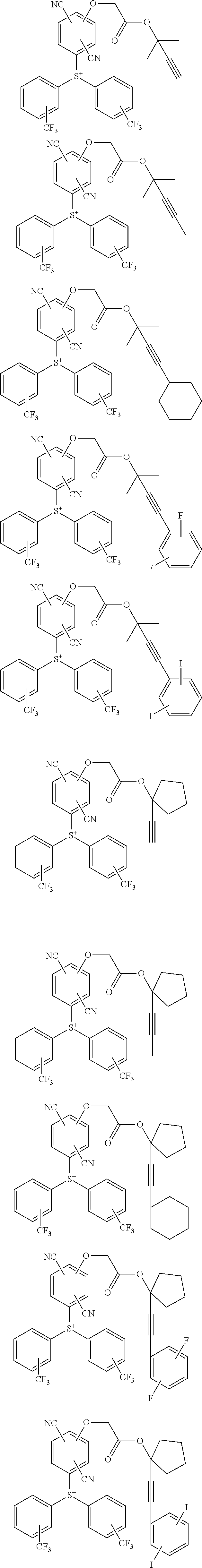

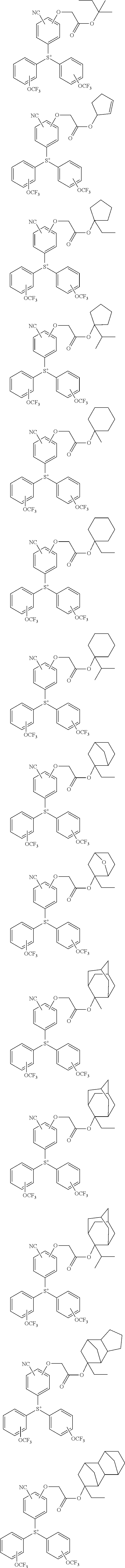

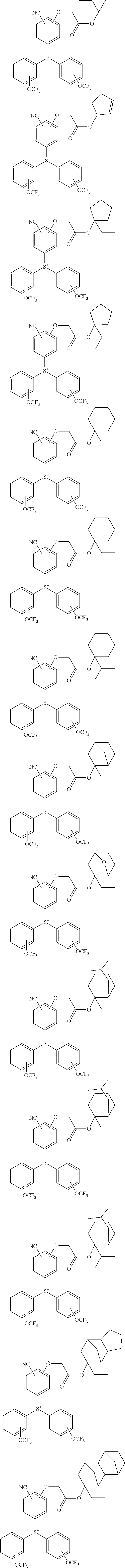

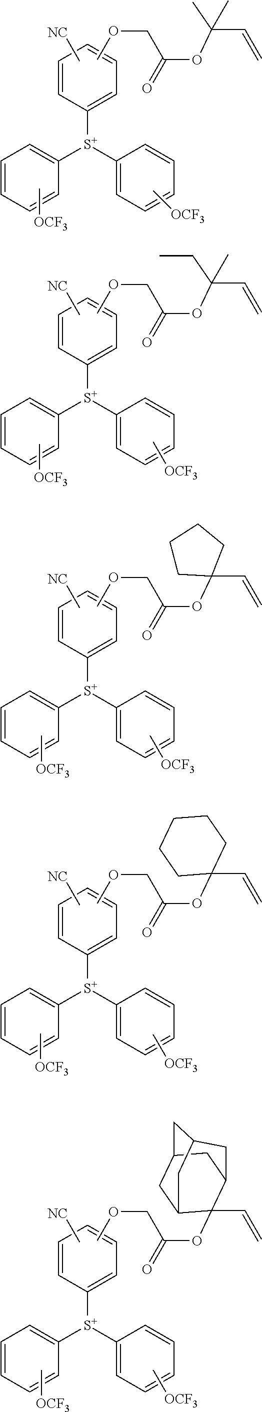

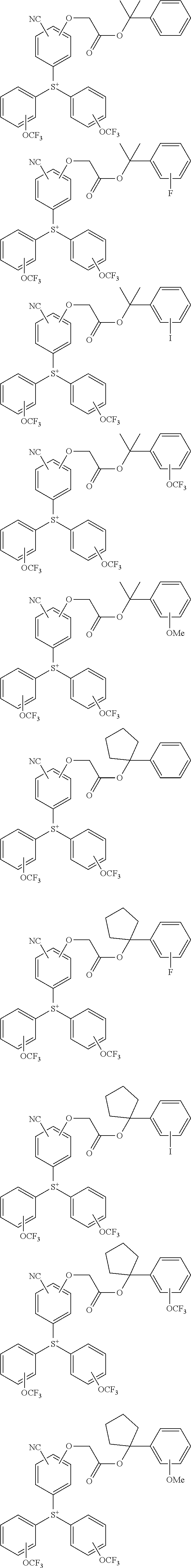

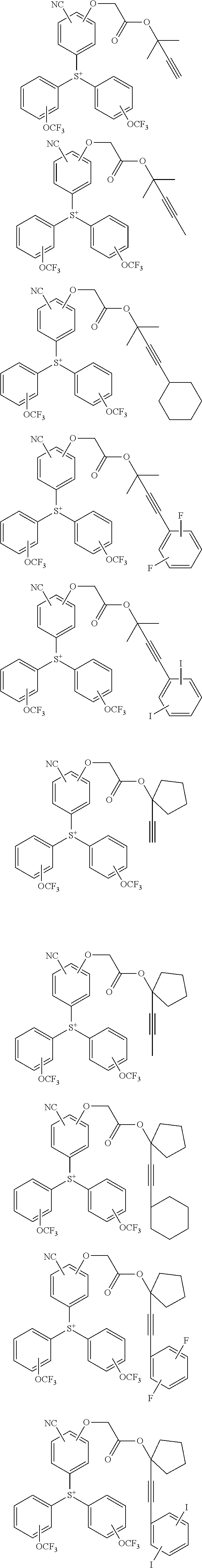

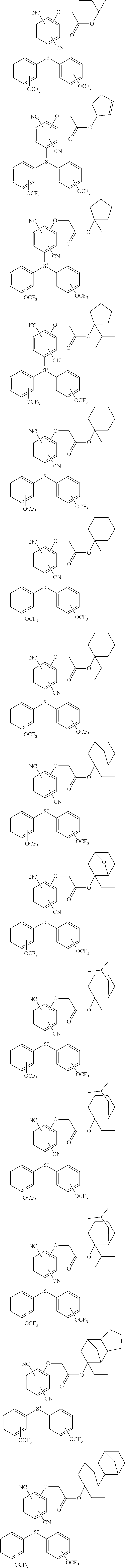

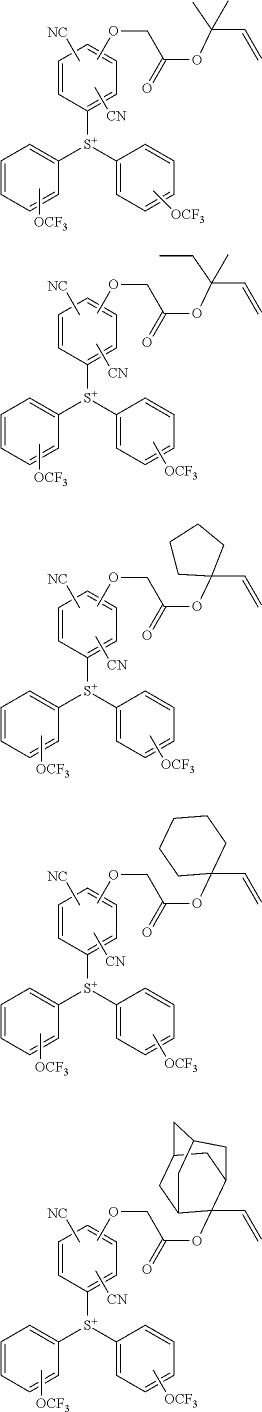

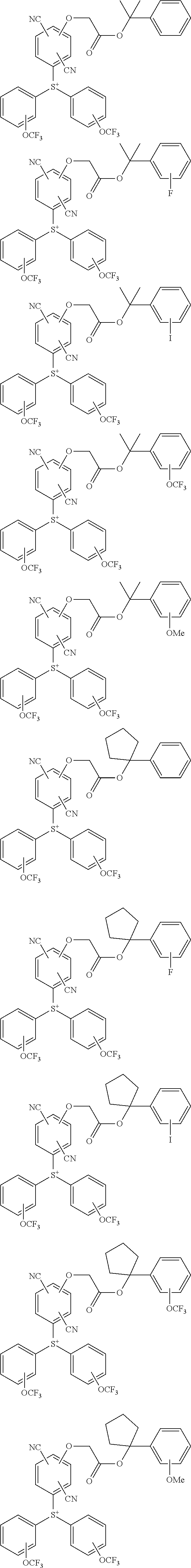

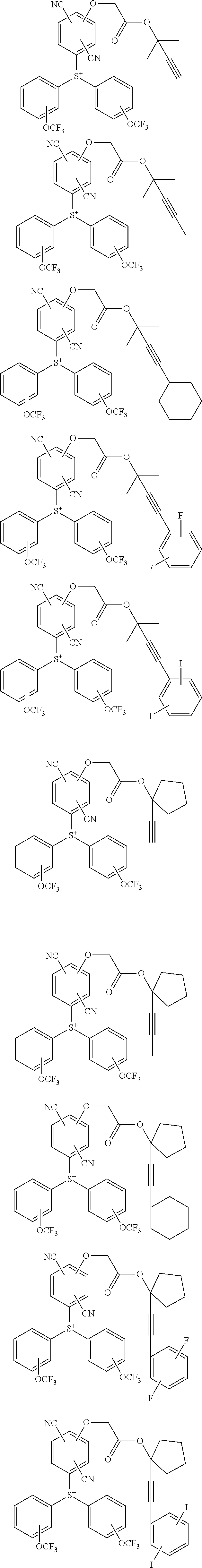

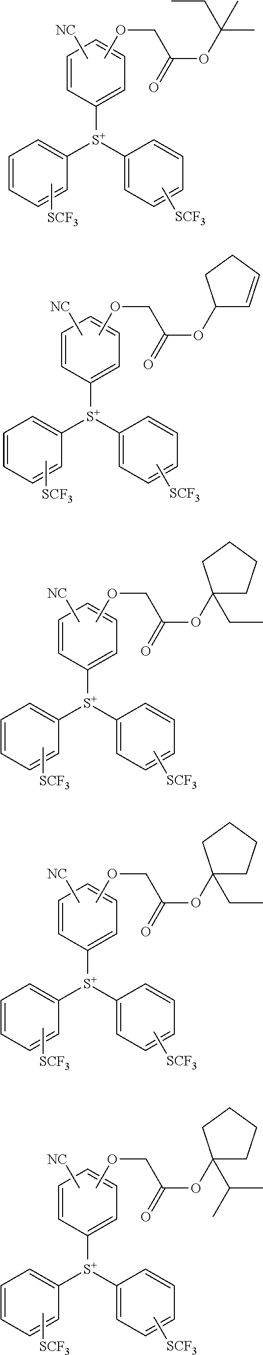

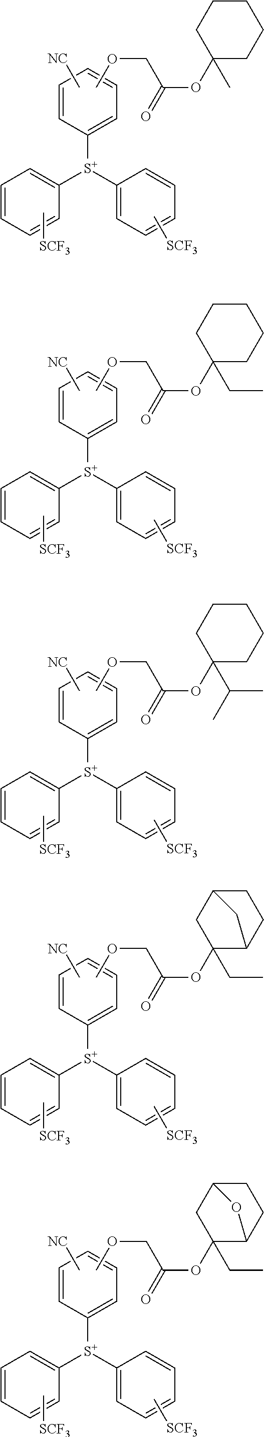

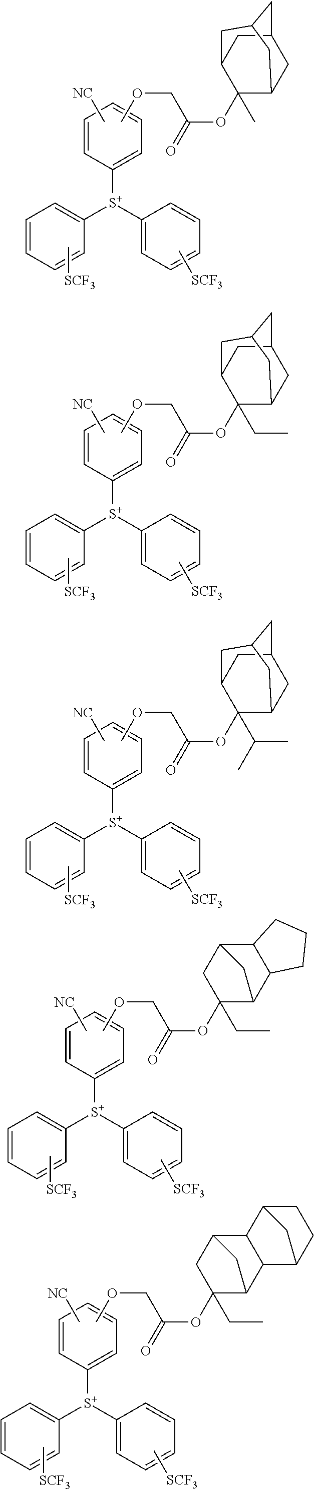

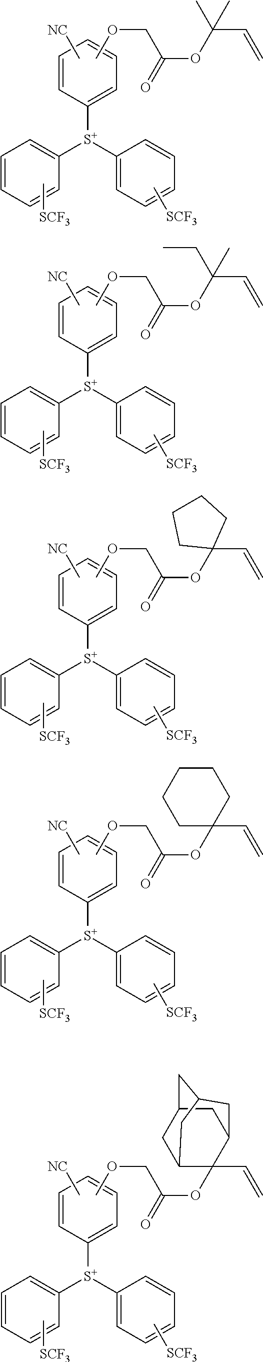

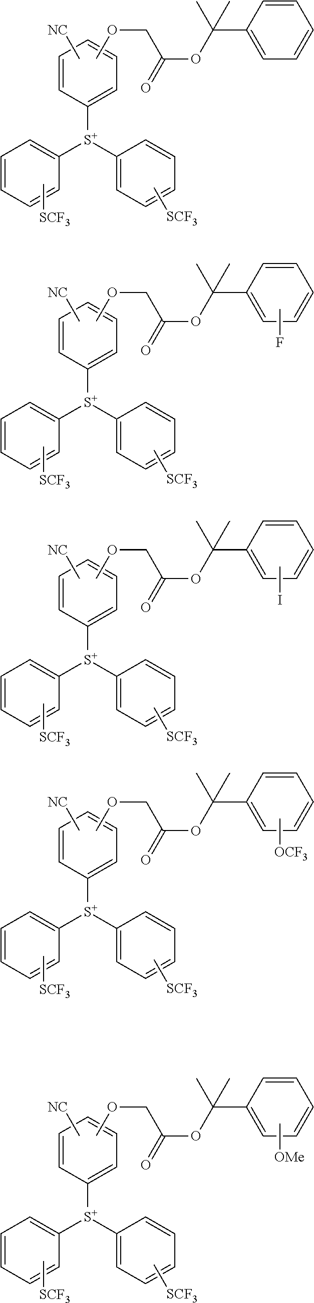

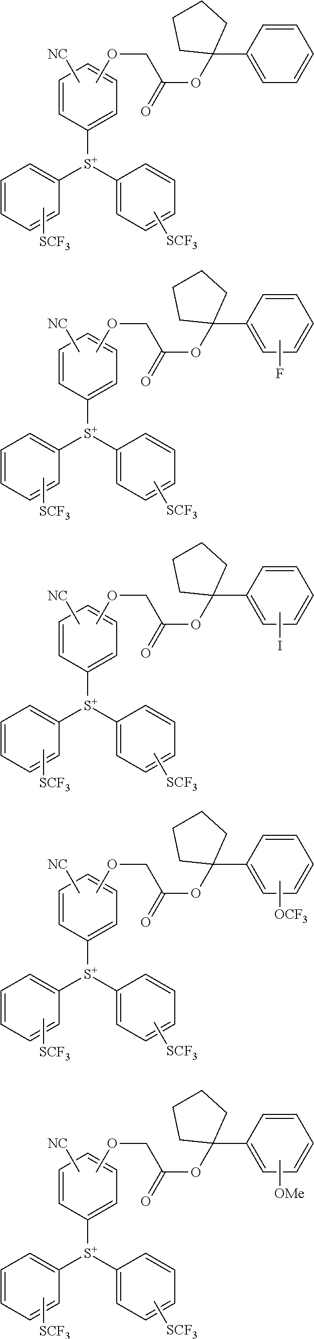

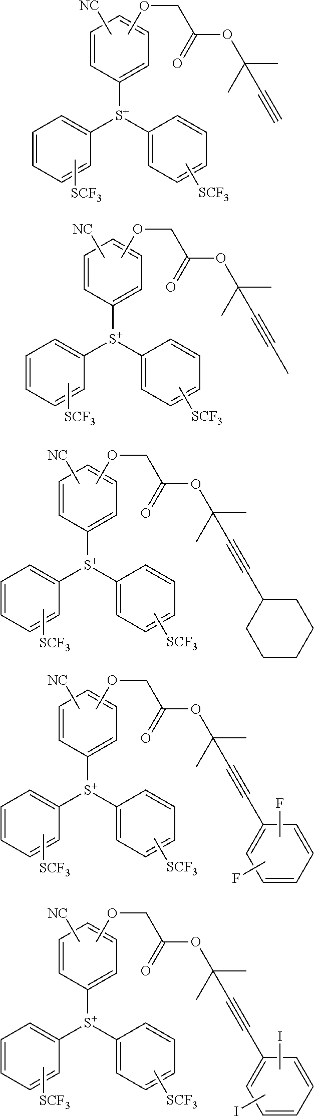

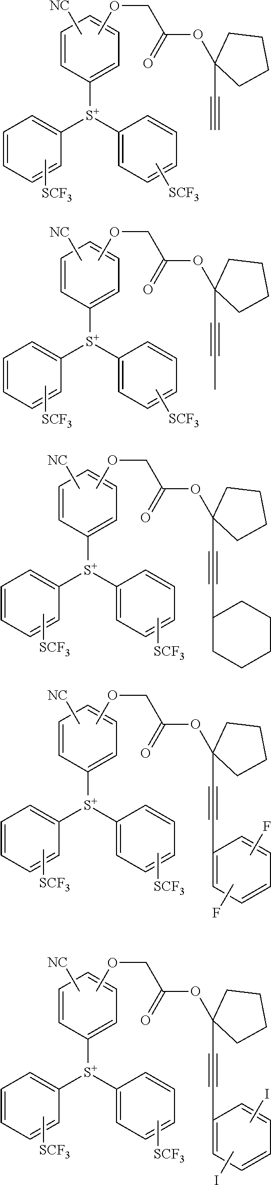

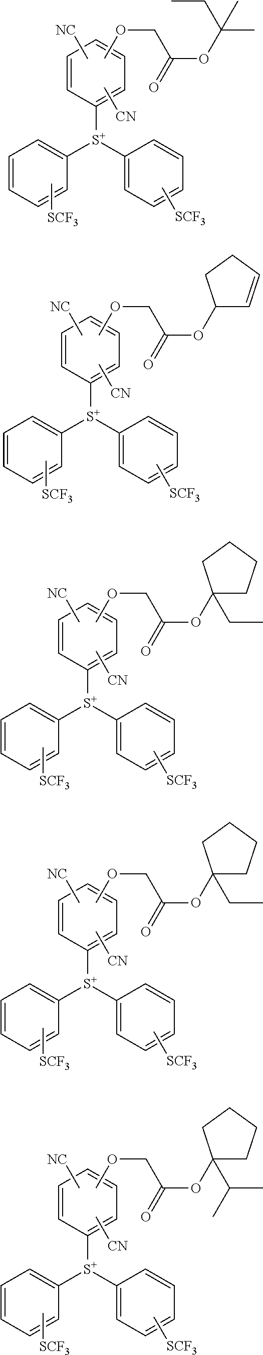

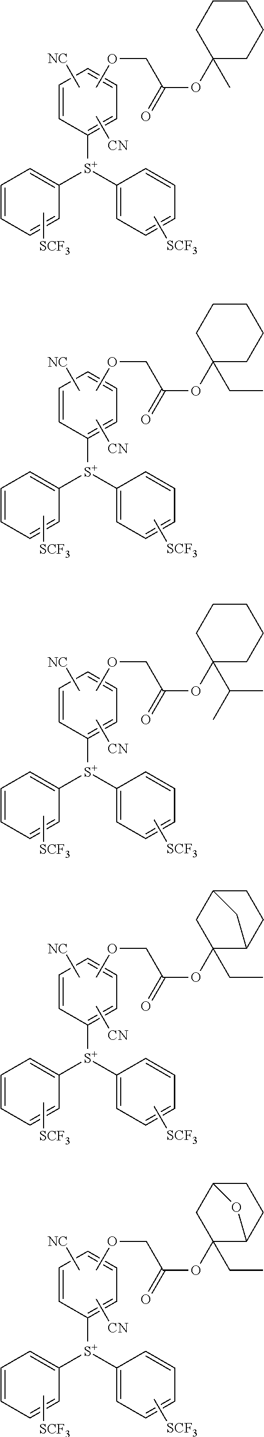

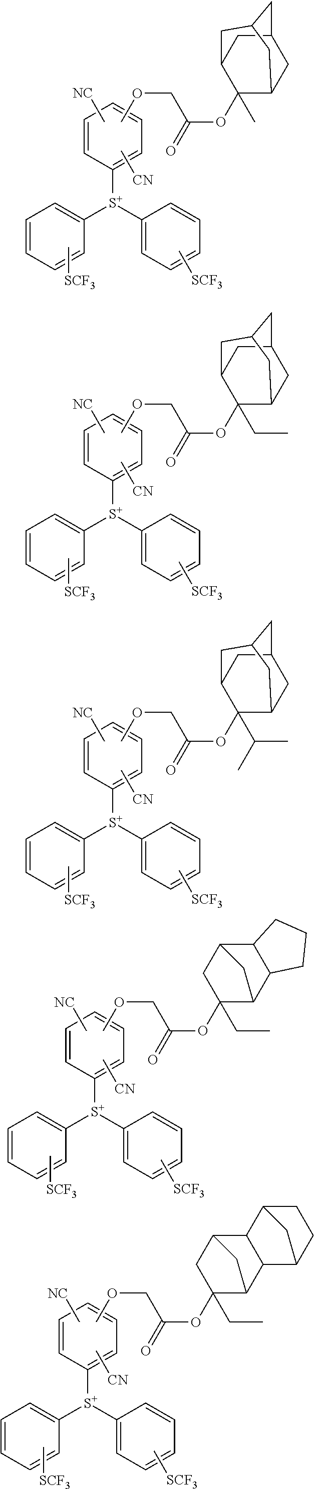

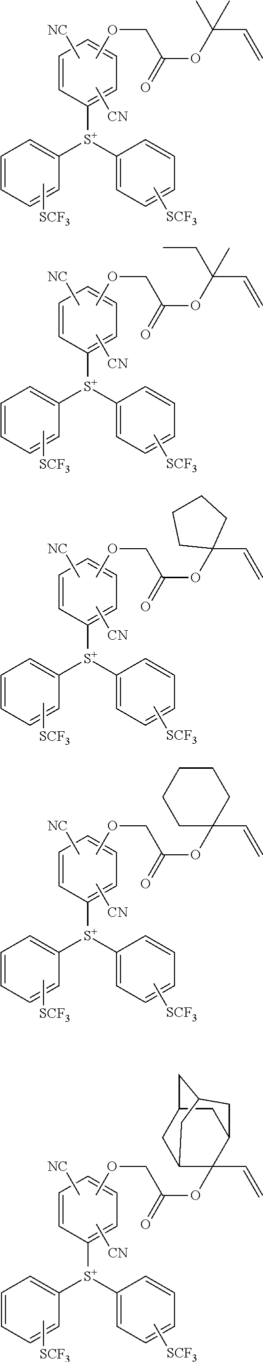

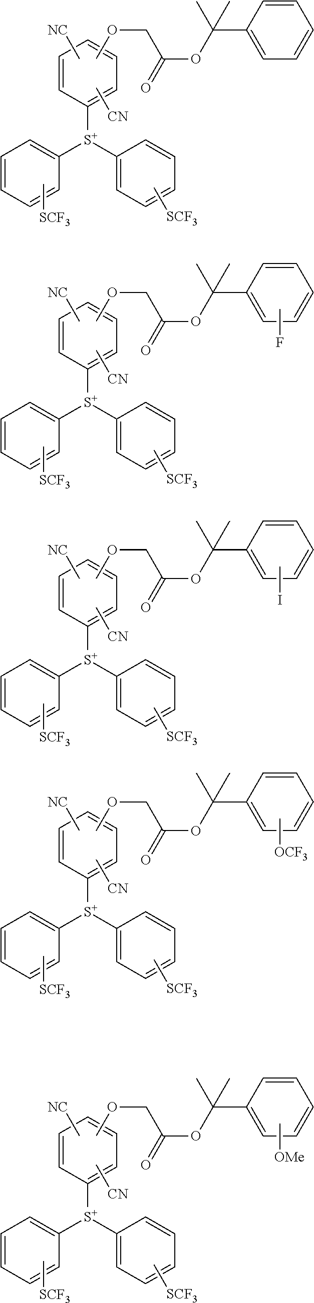

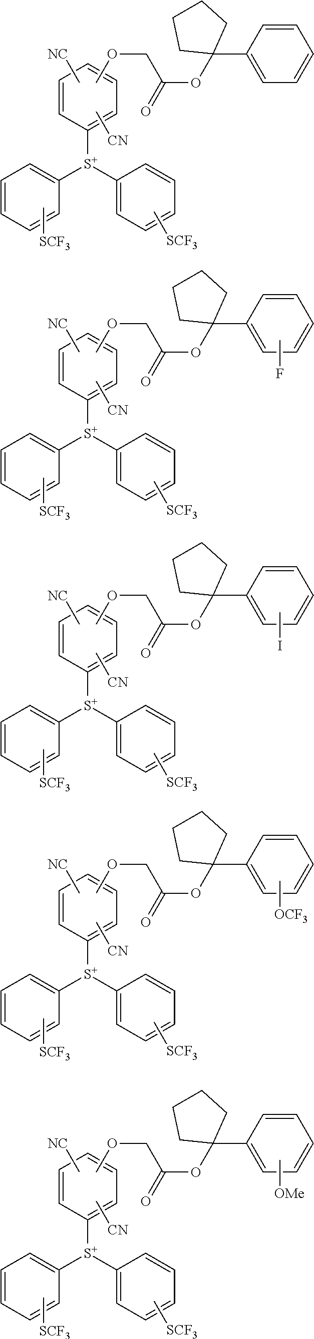

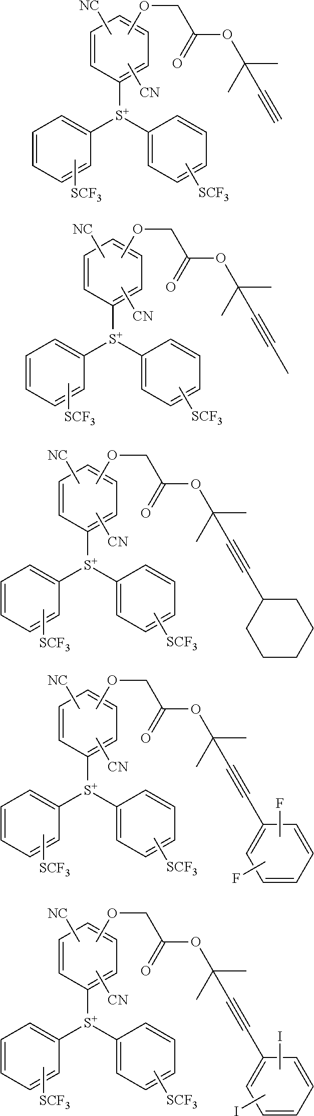

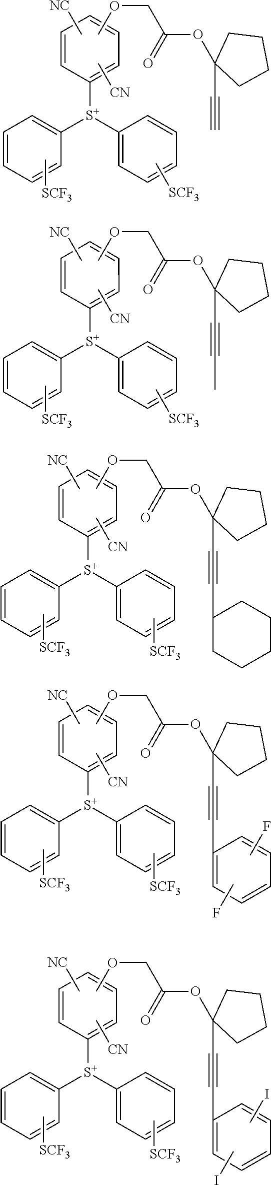

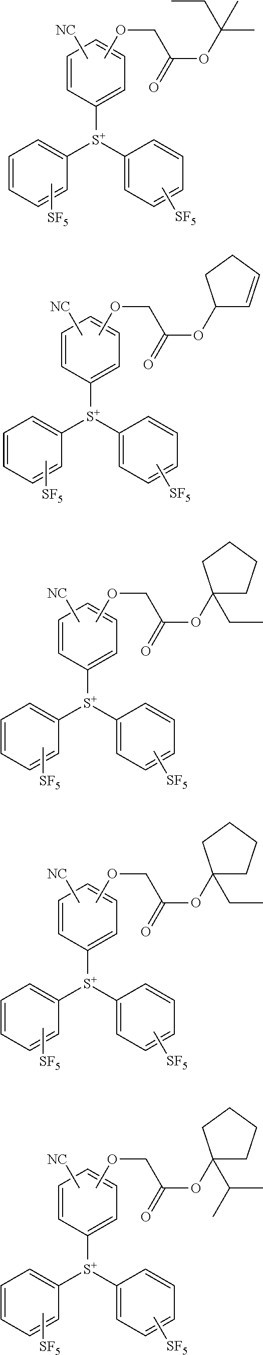

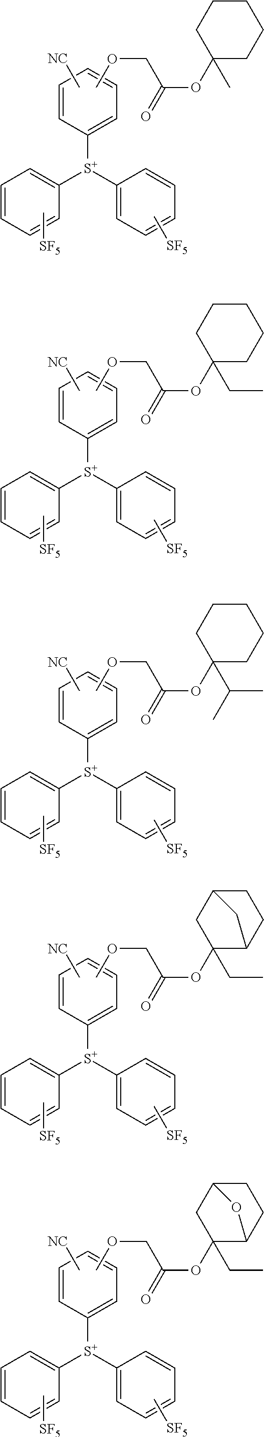

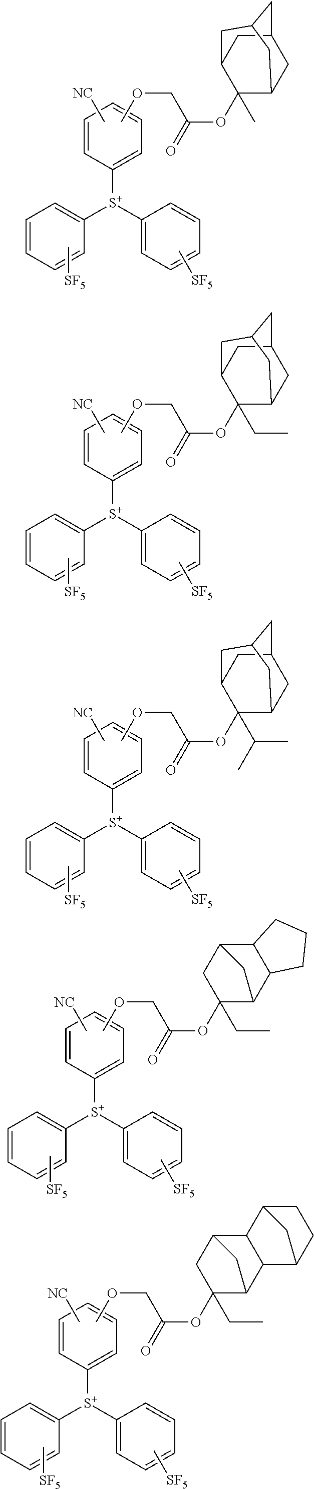

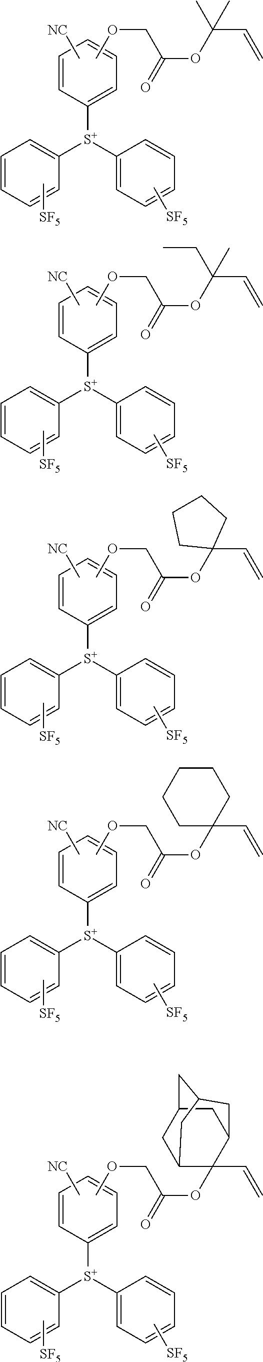

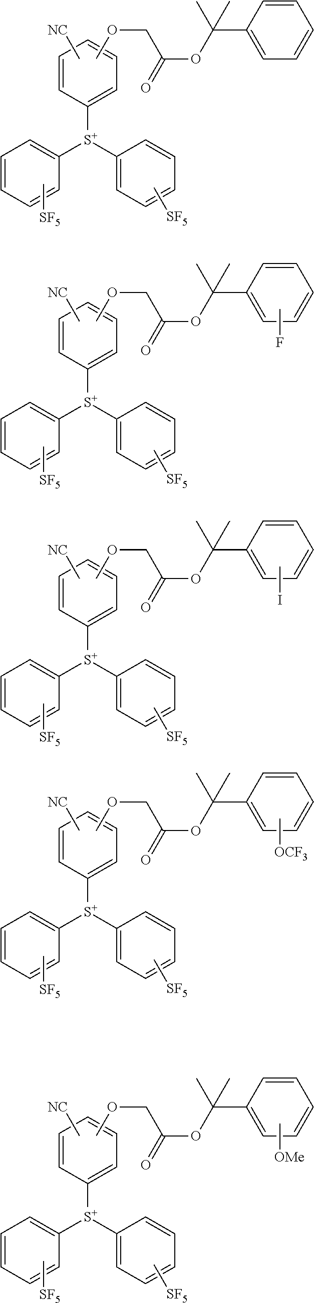

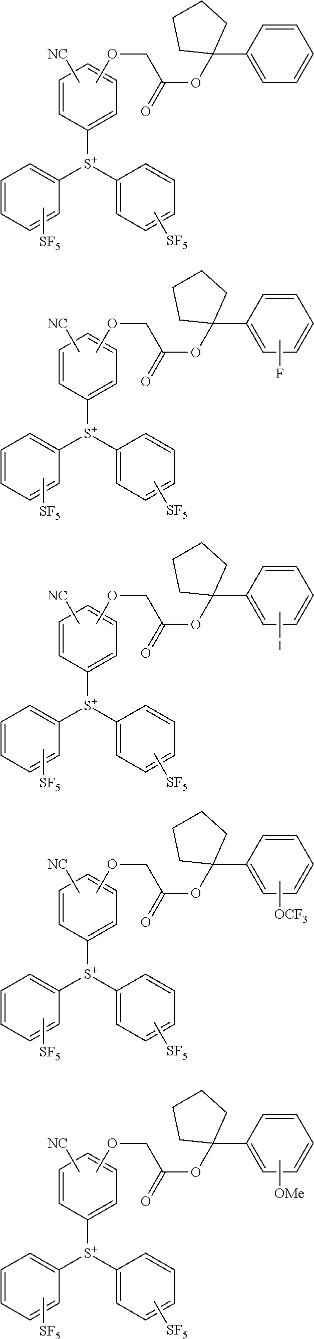

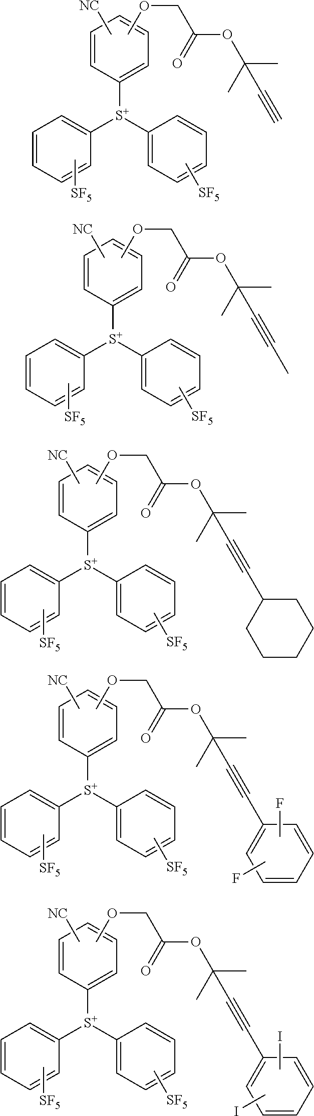

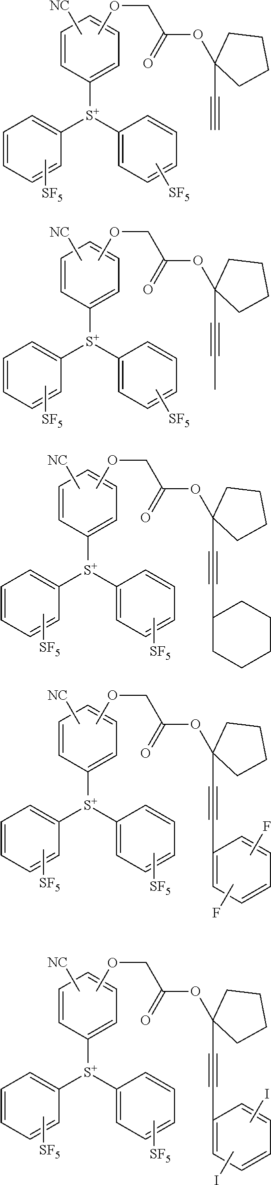

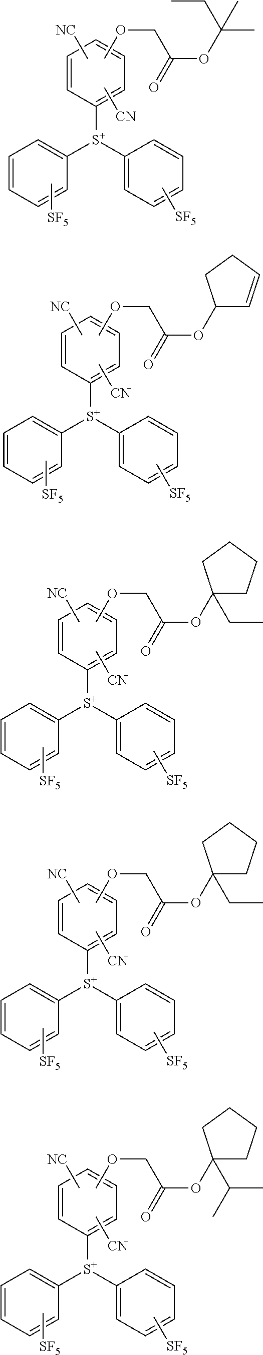

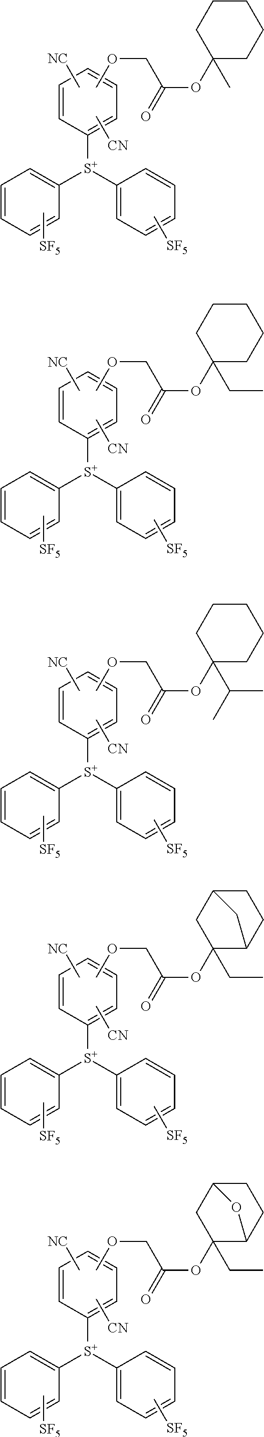

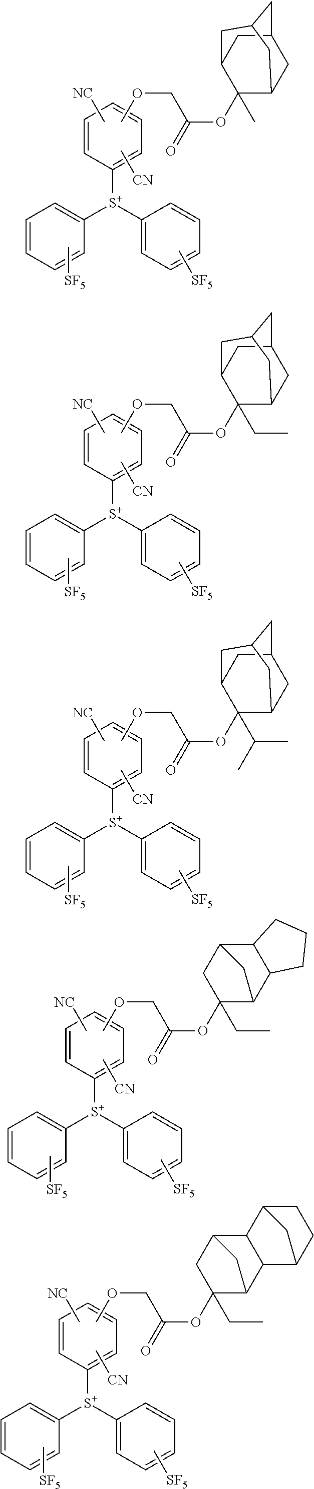

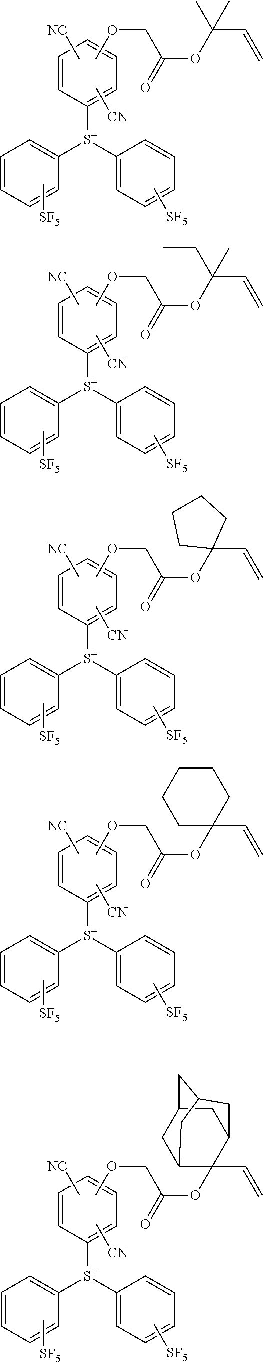

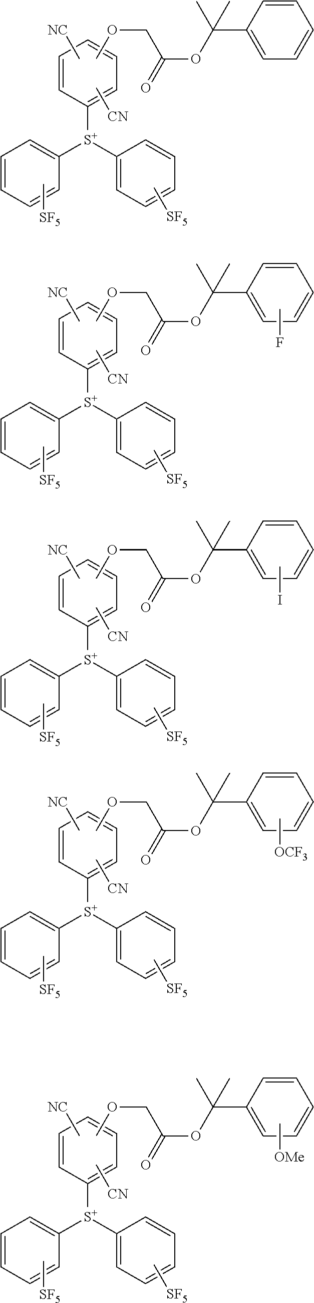

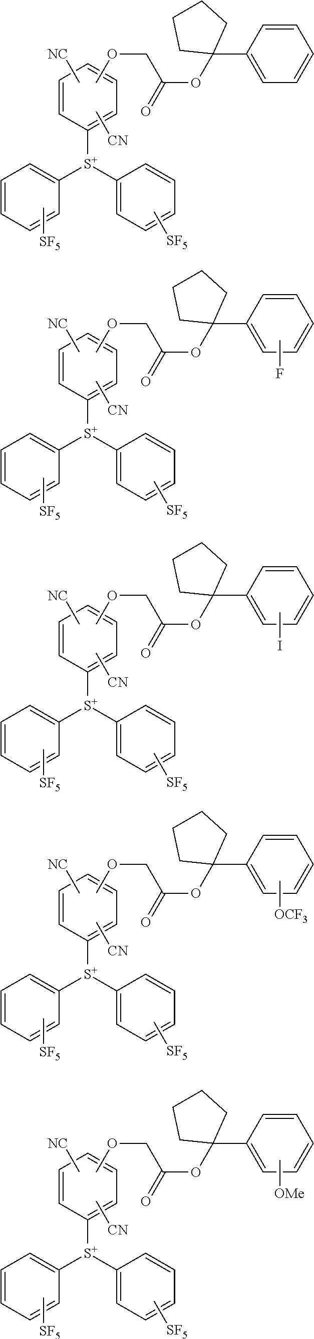

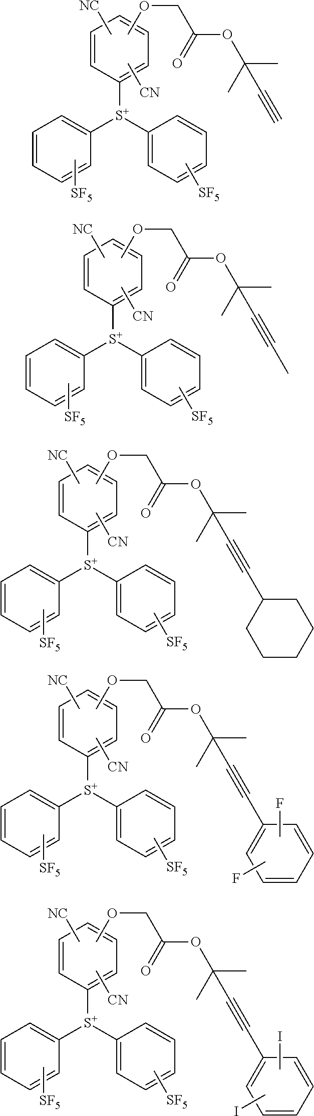

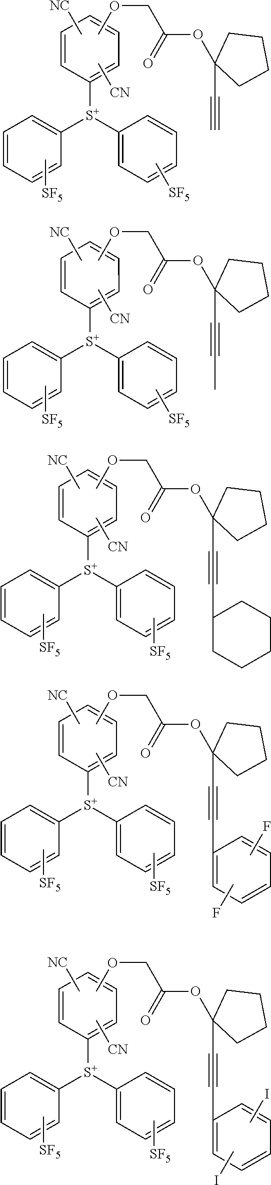

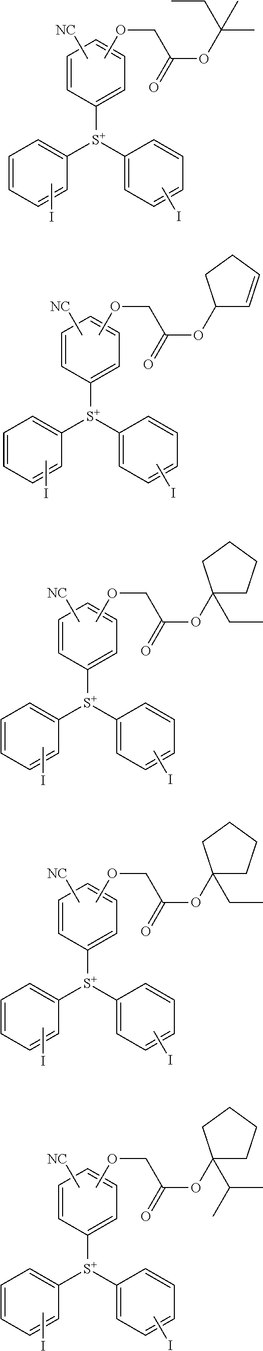

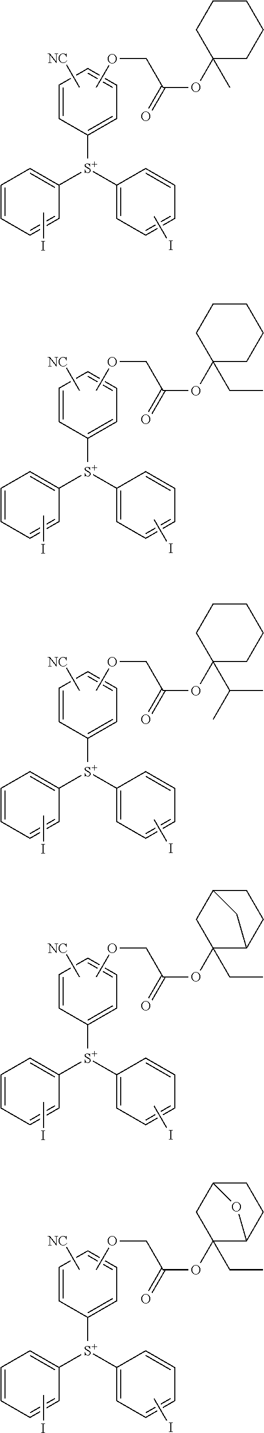

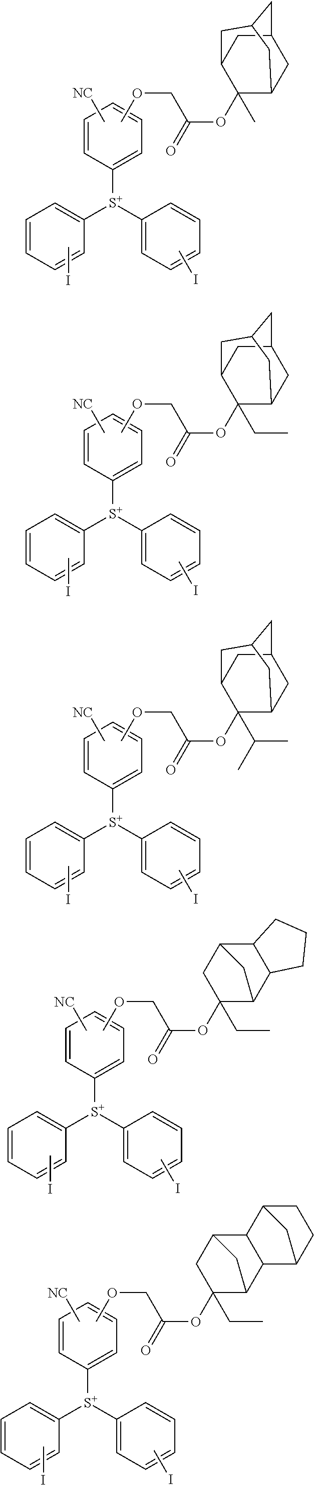

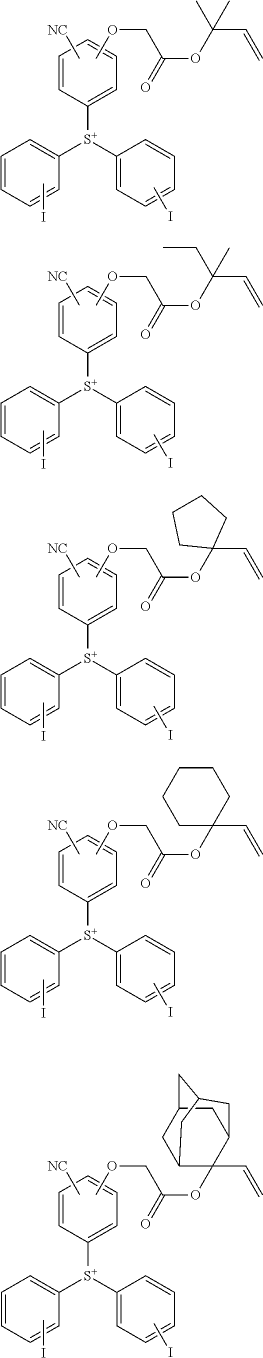

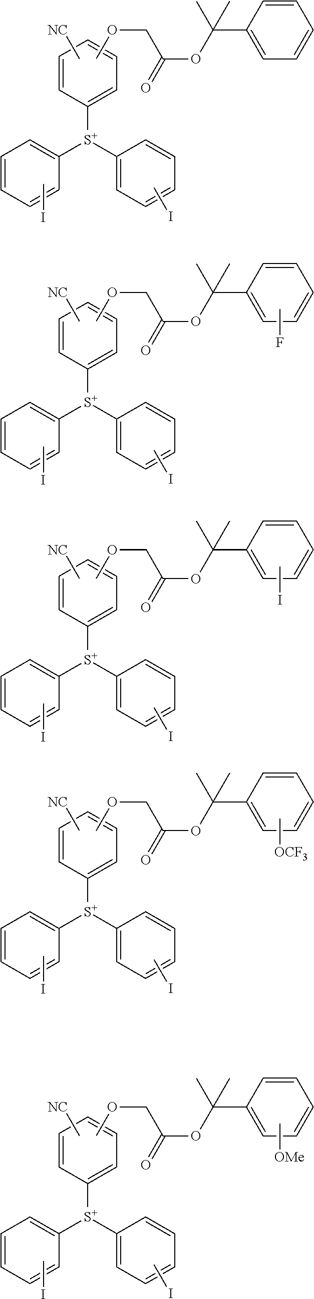

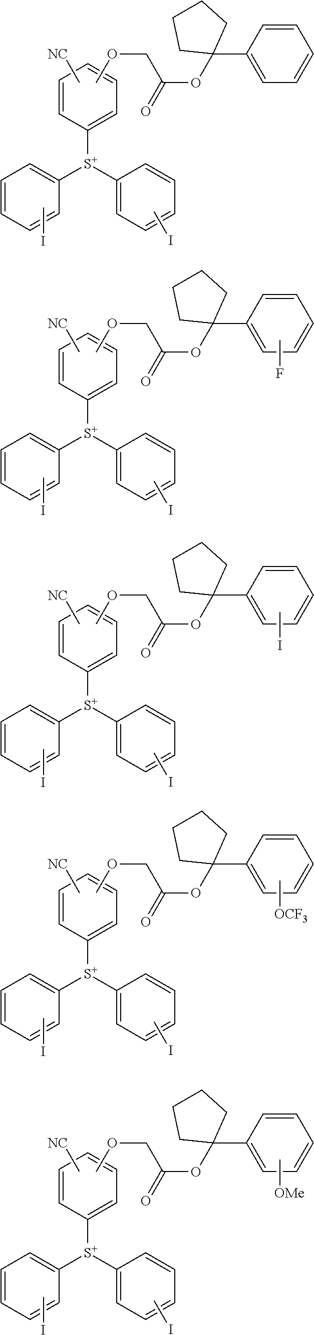

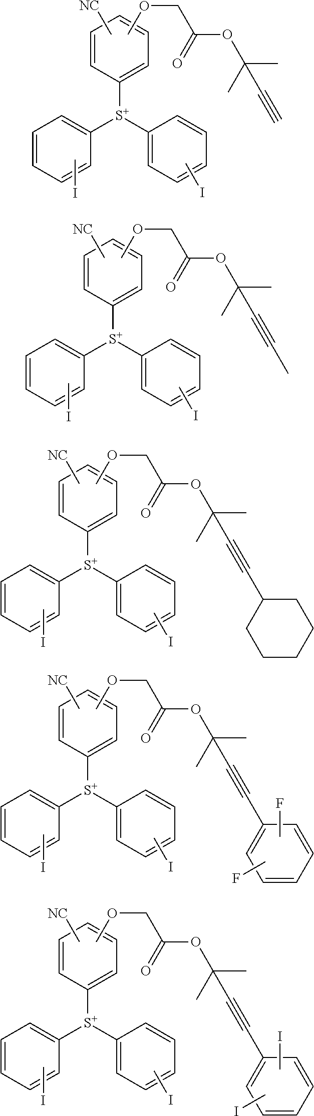

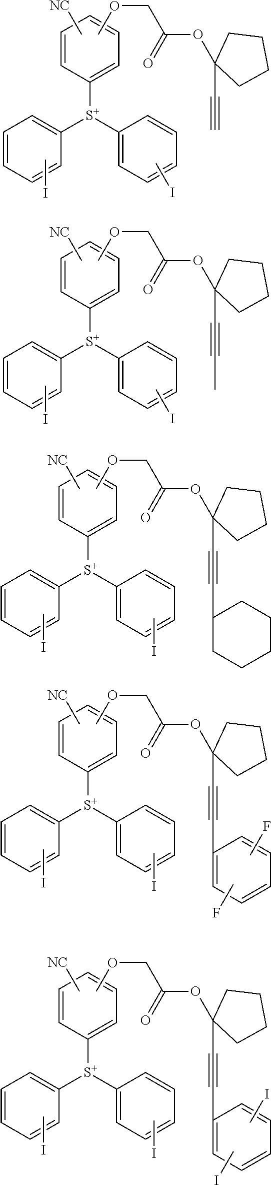

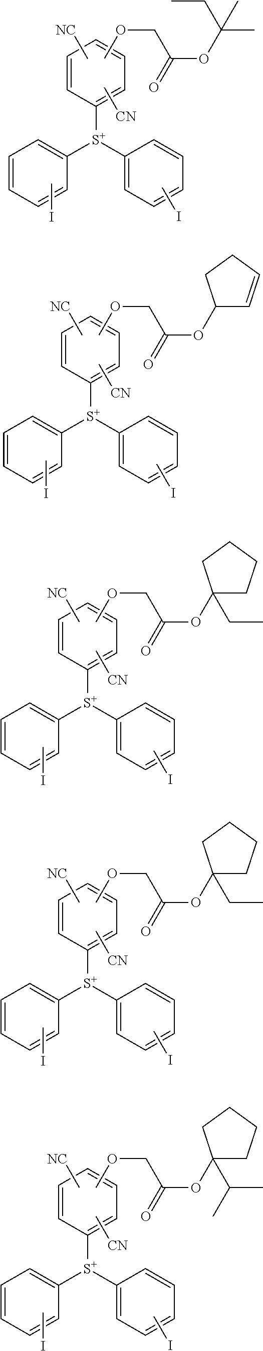

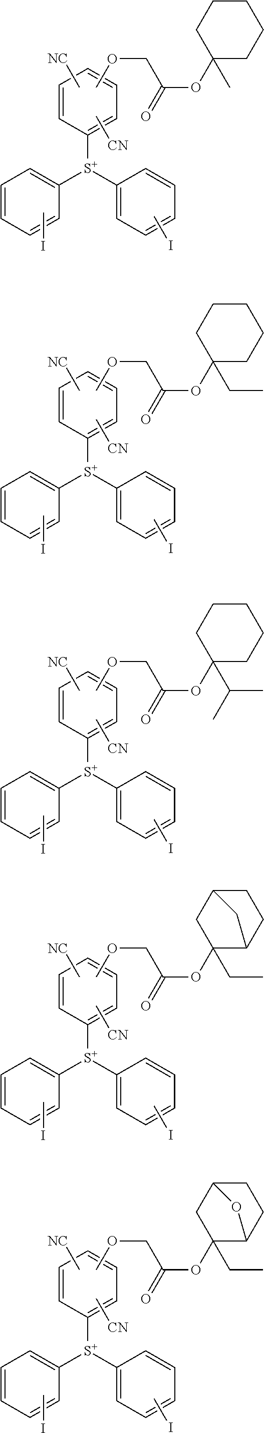

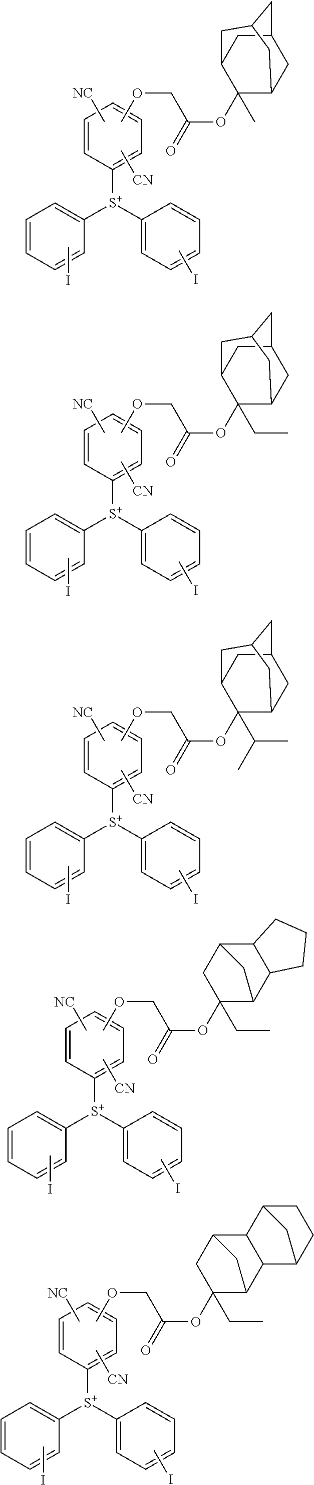

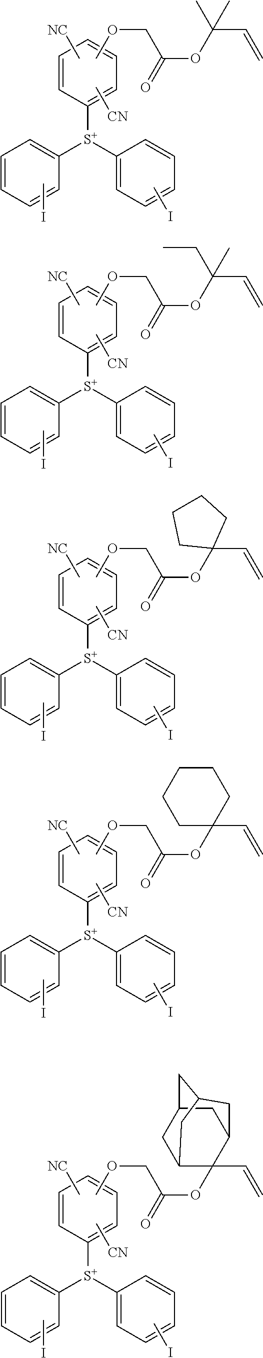

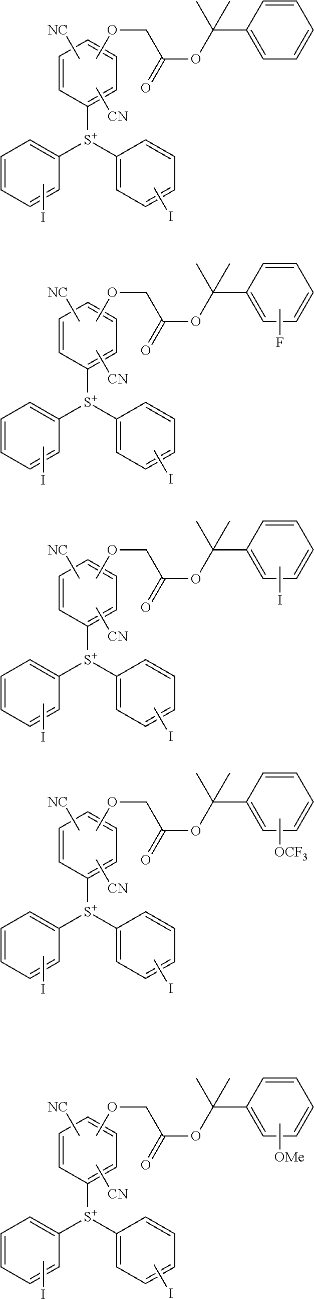

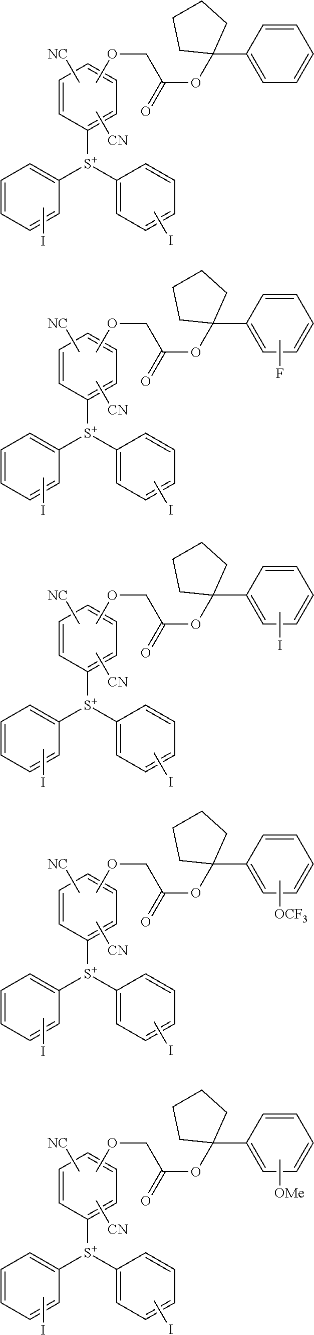

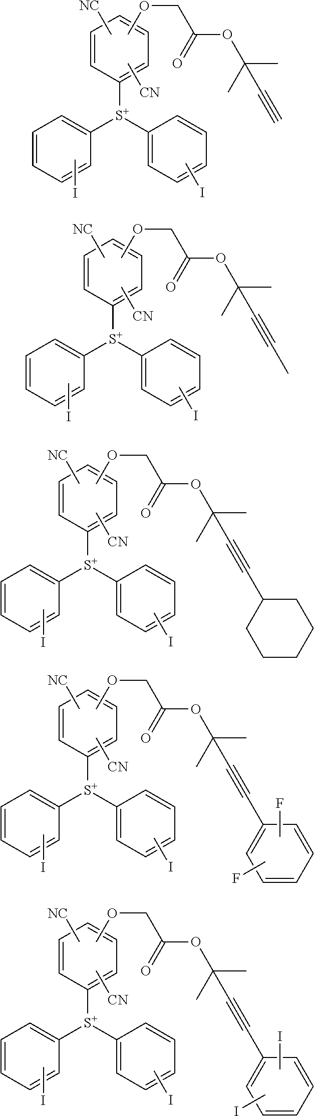

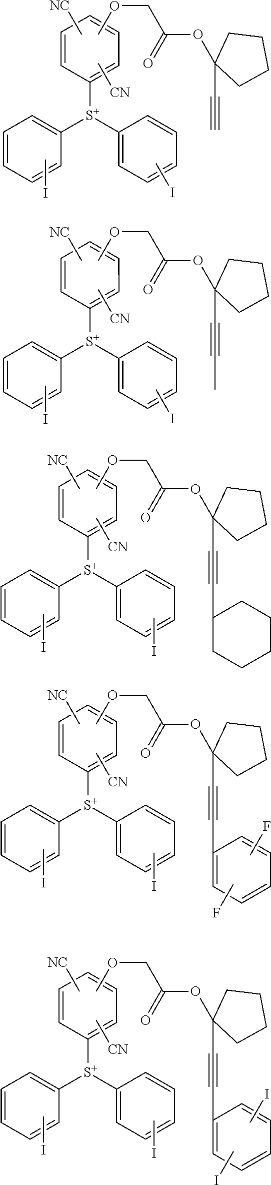

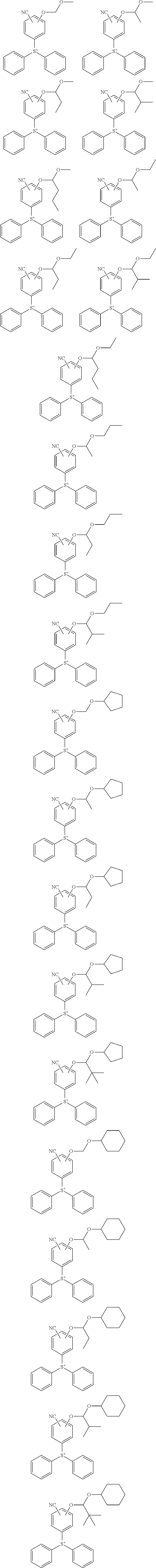

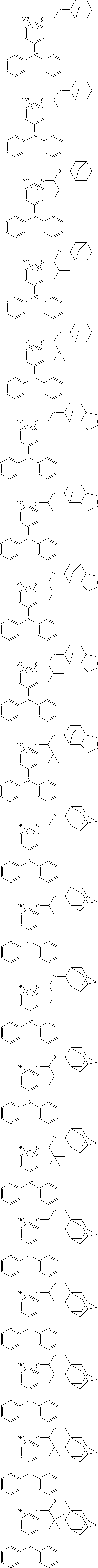

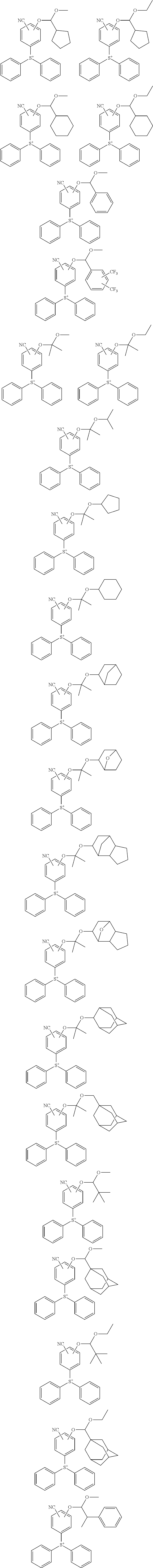

[chemical structure]

[chemical structure]

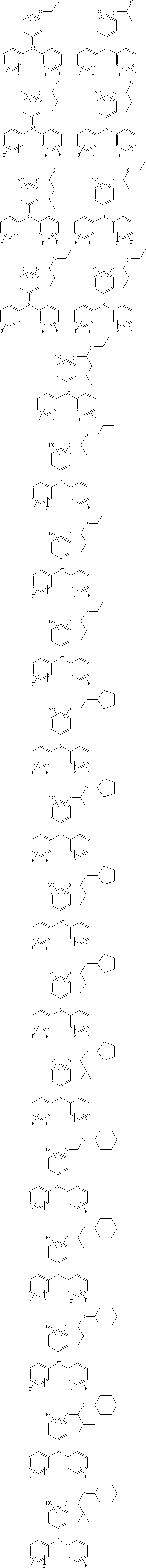

[chemical structure]

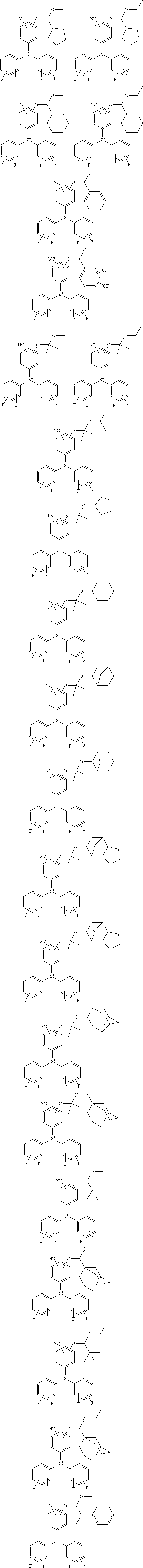

[chemical structure]

[chemical structure]

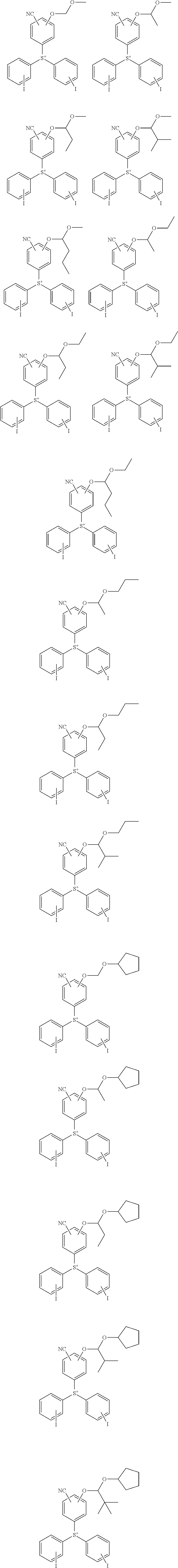

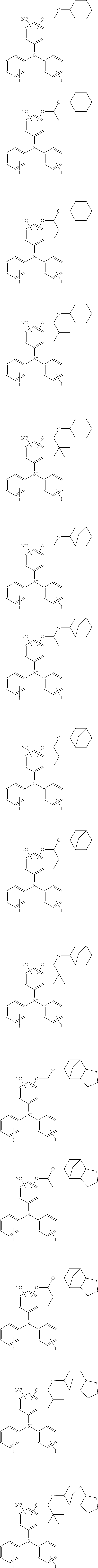

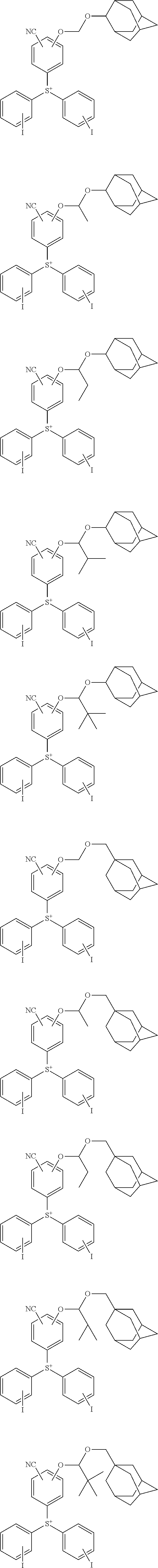

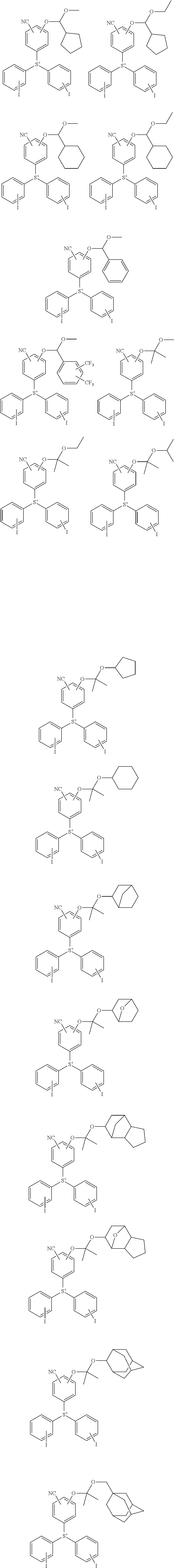

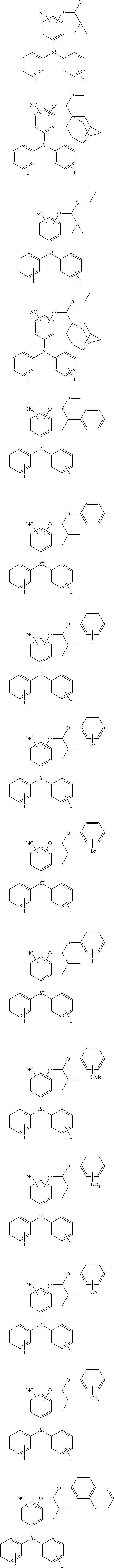

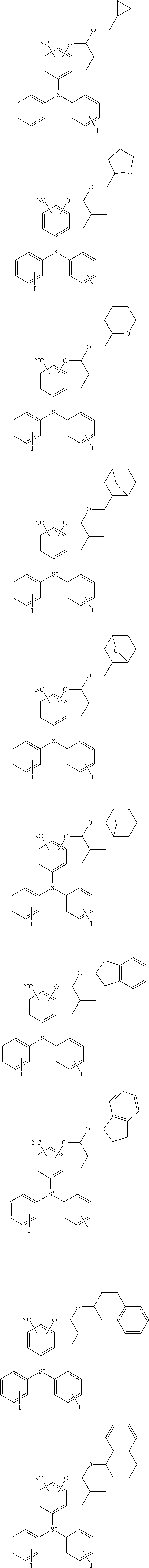

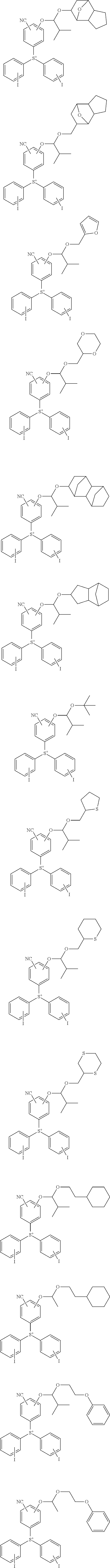

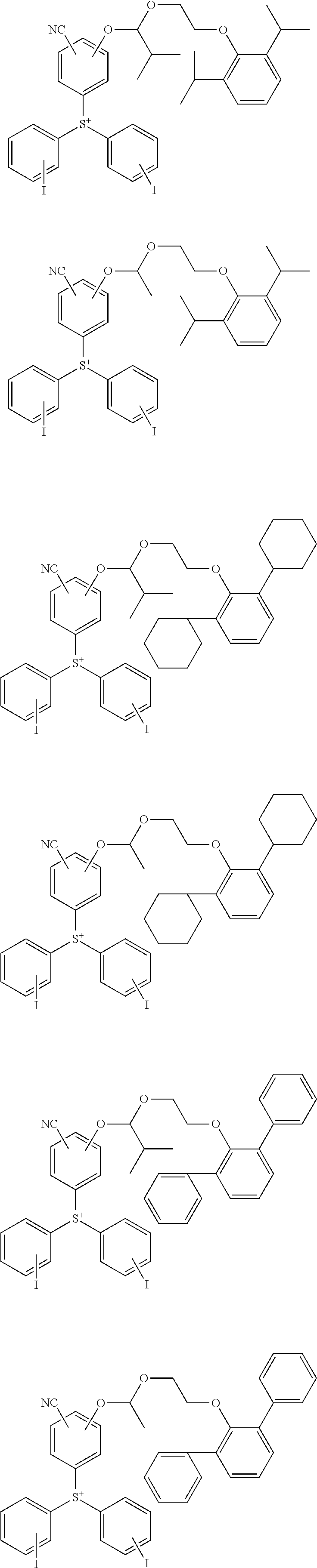

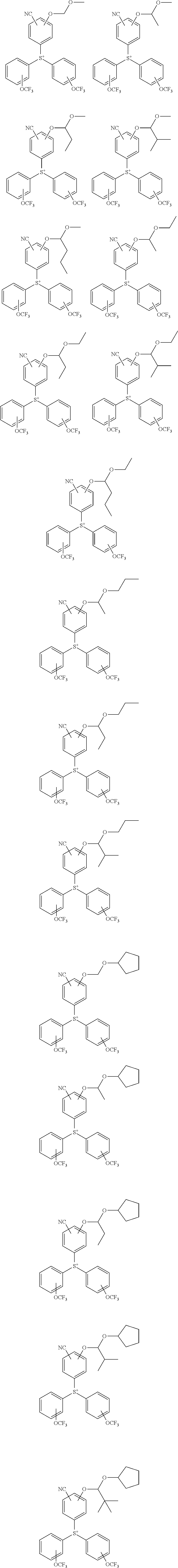

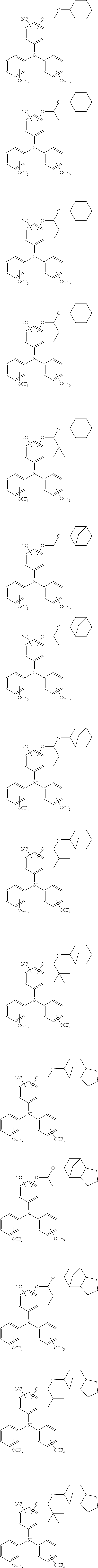

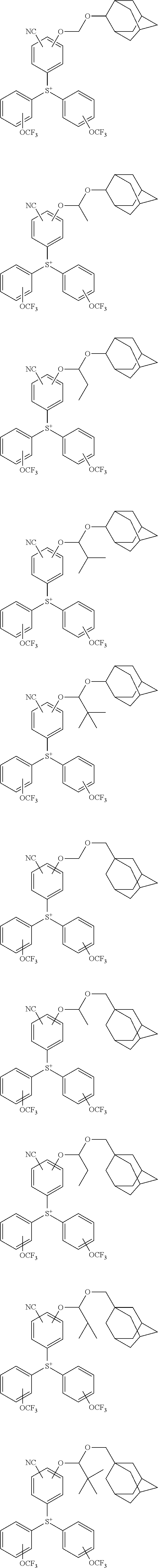

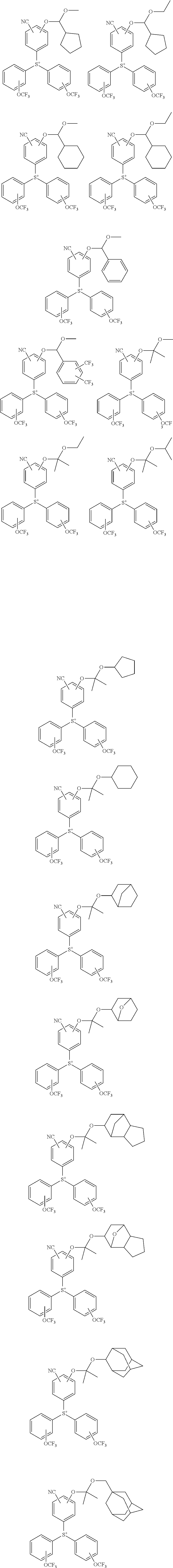

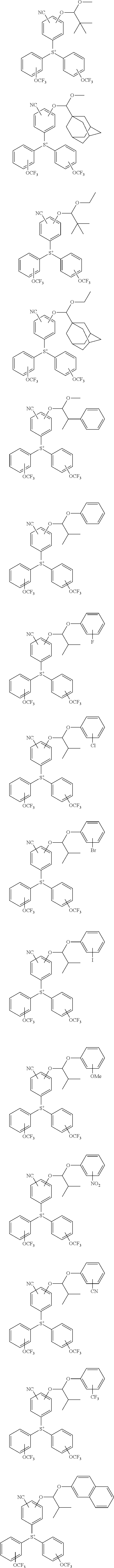

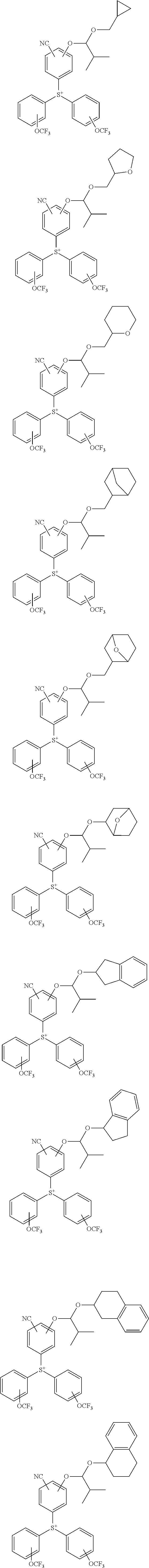

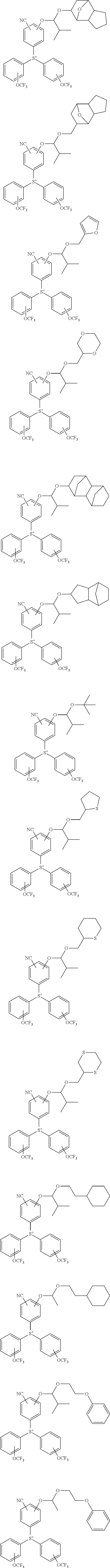

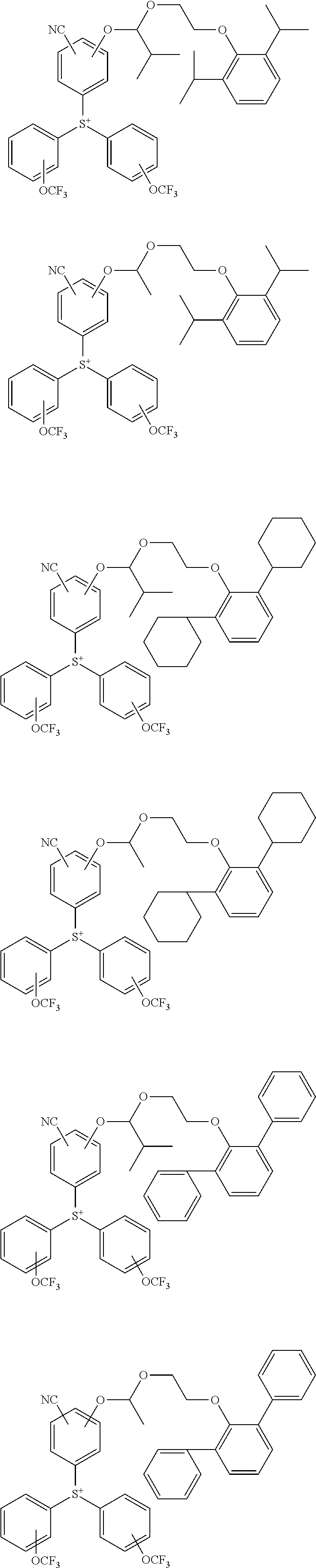

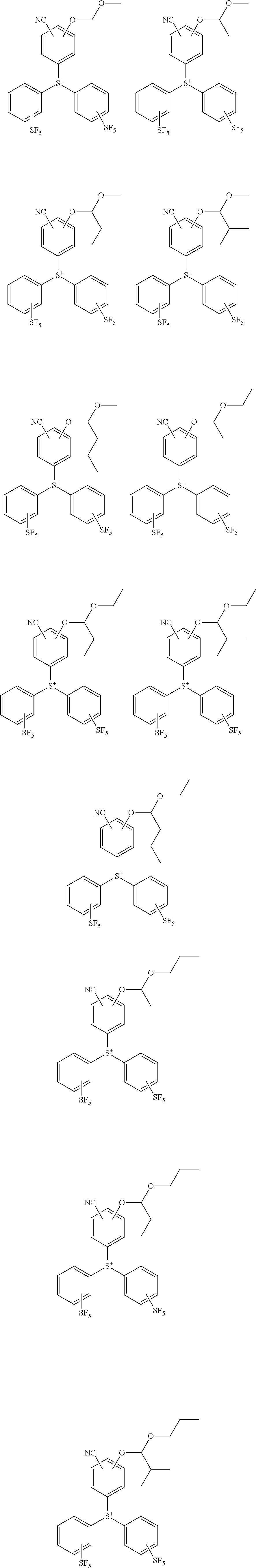

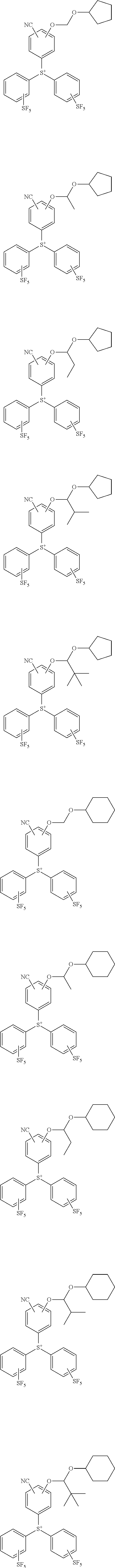

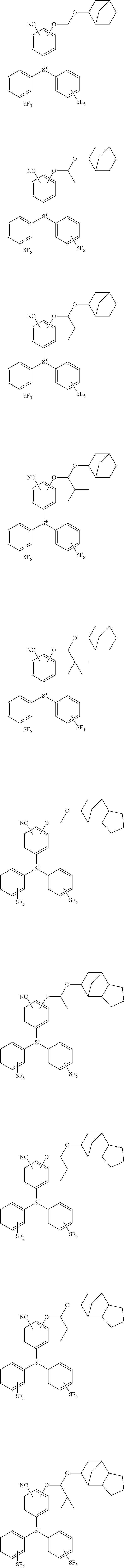

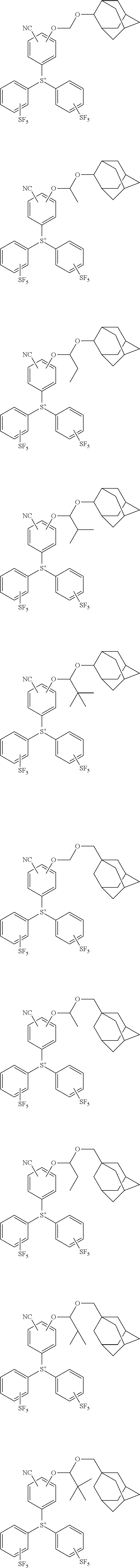

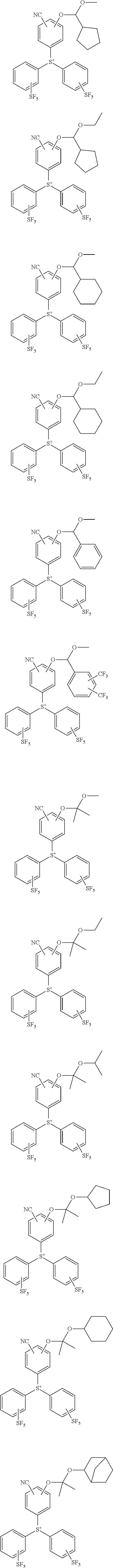

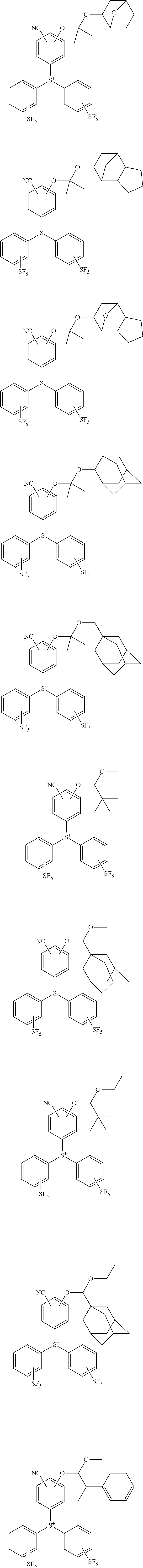

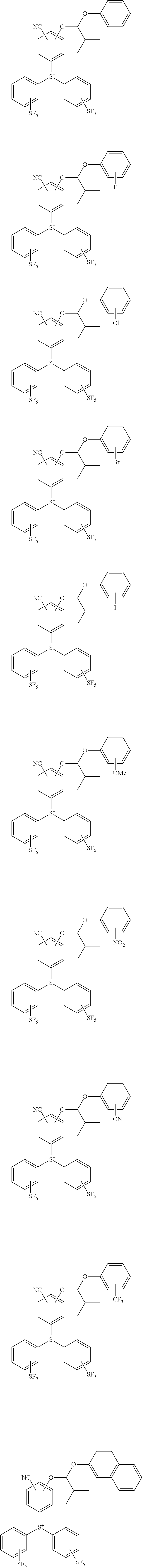

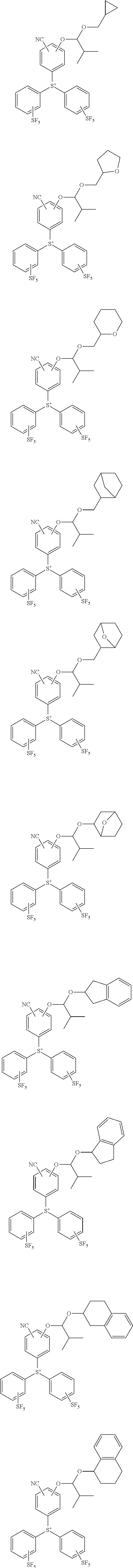

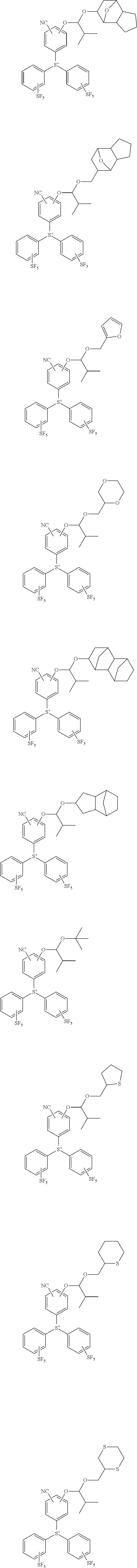

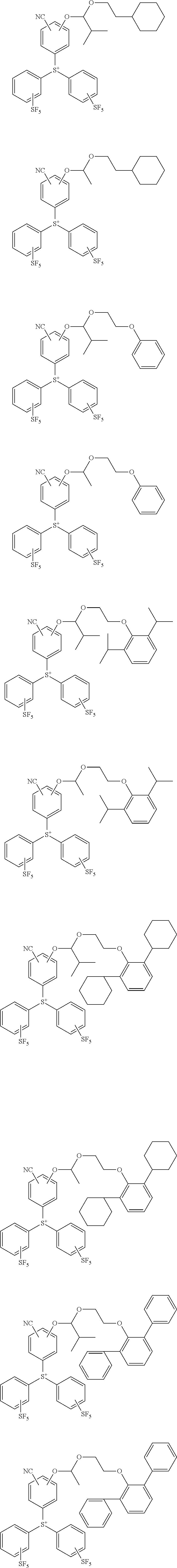

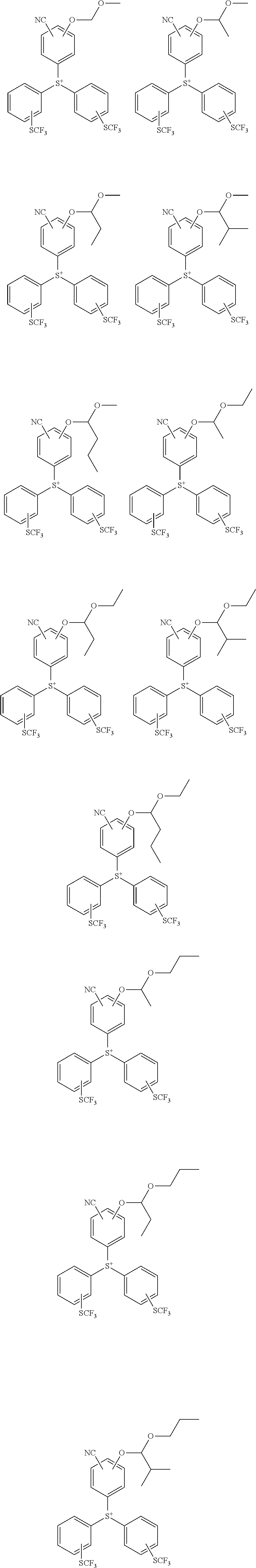

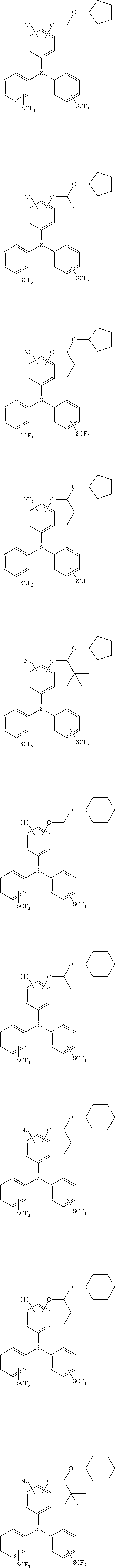

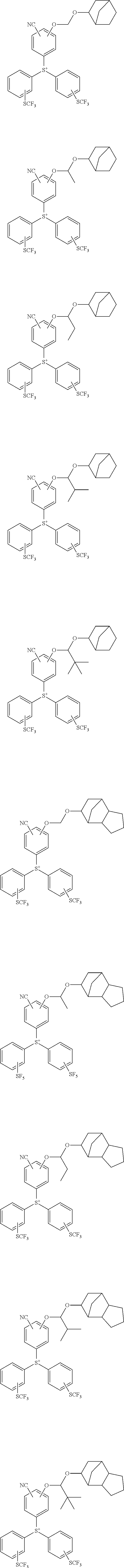

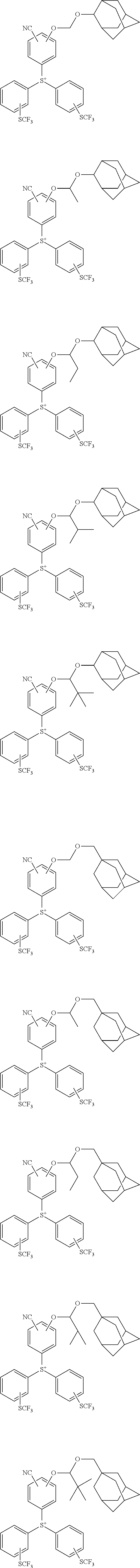

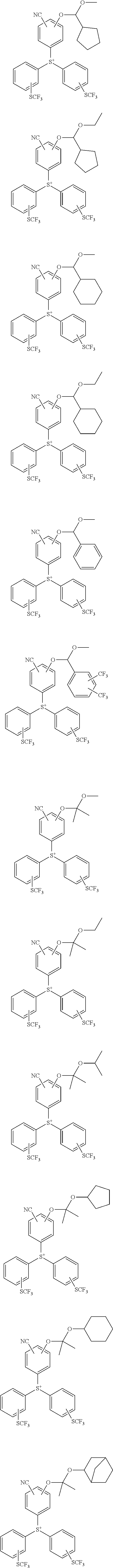

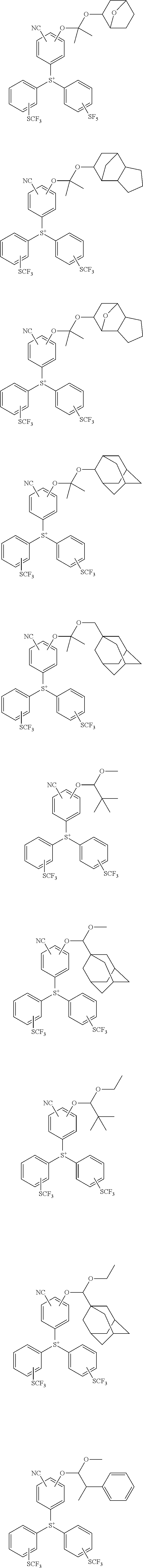

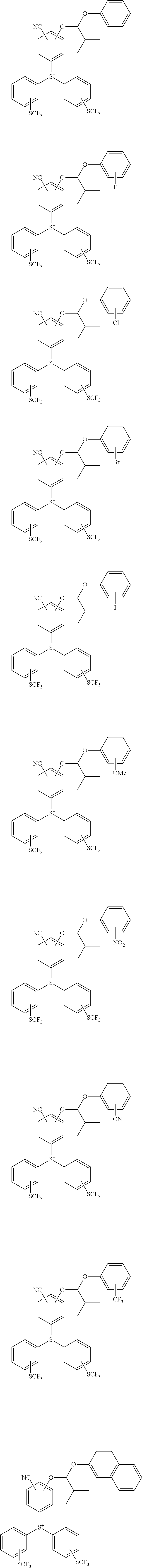

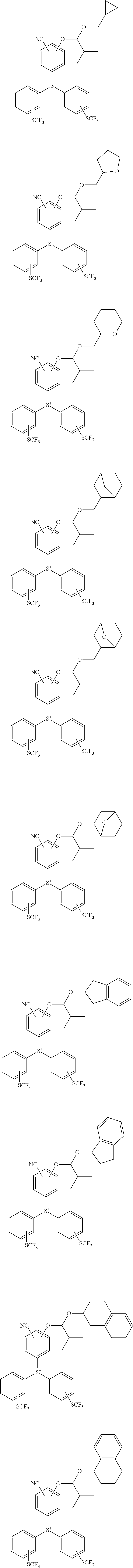

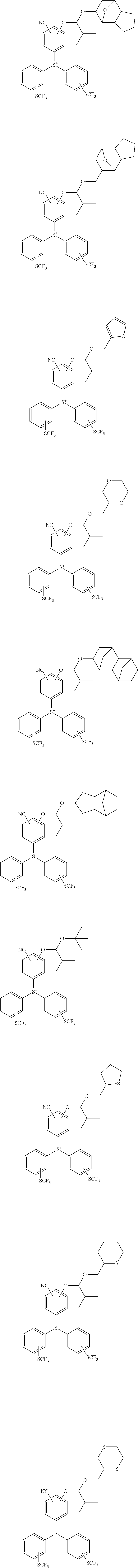

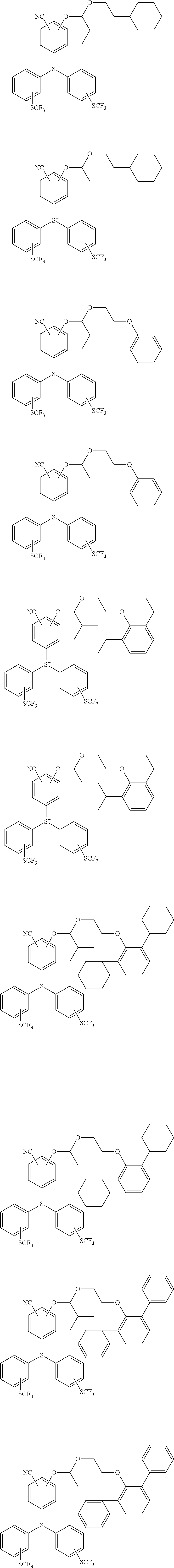

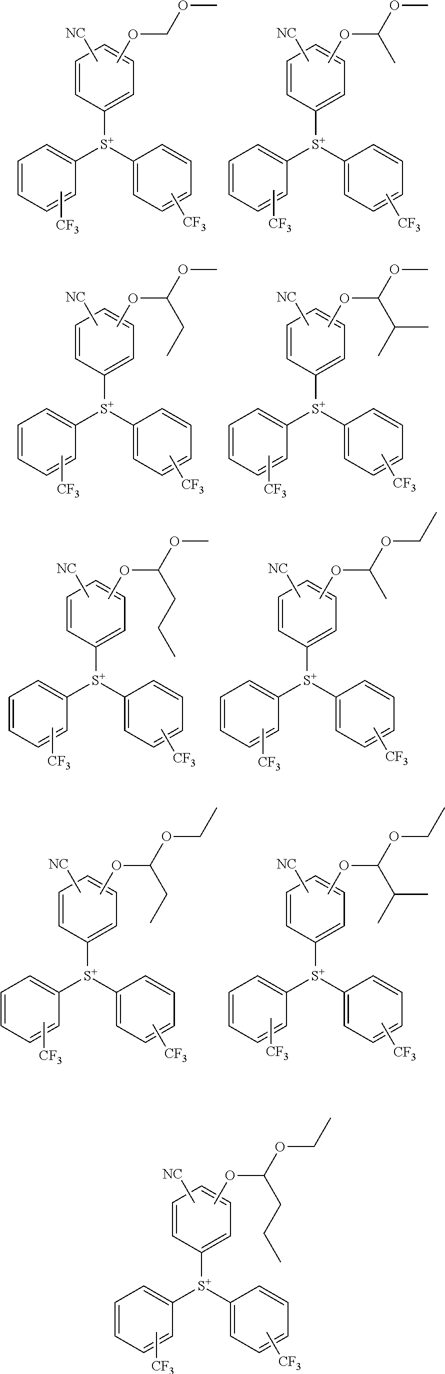

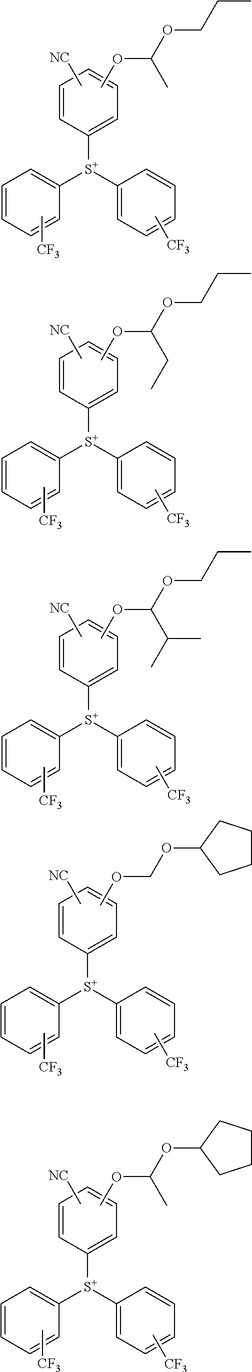

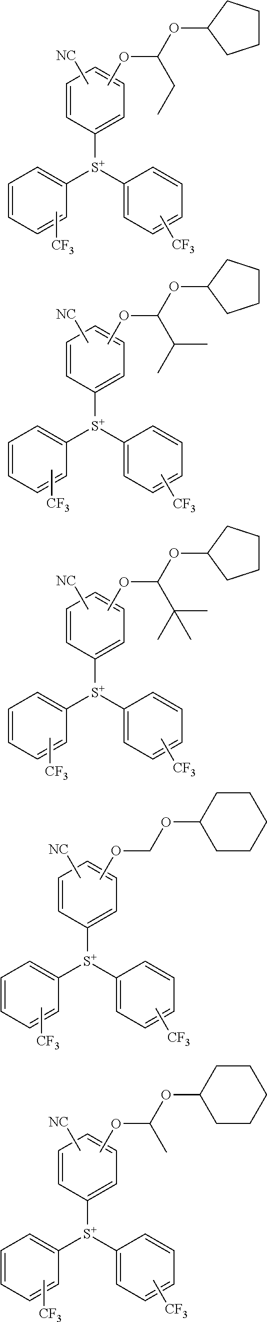

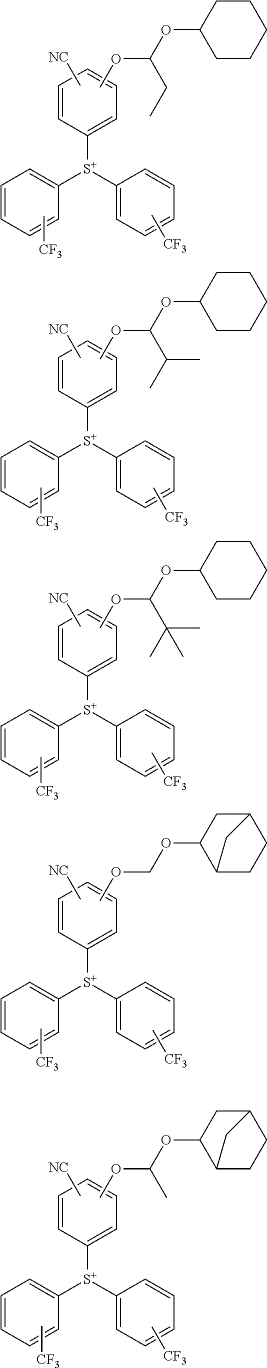

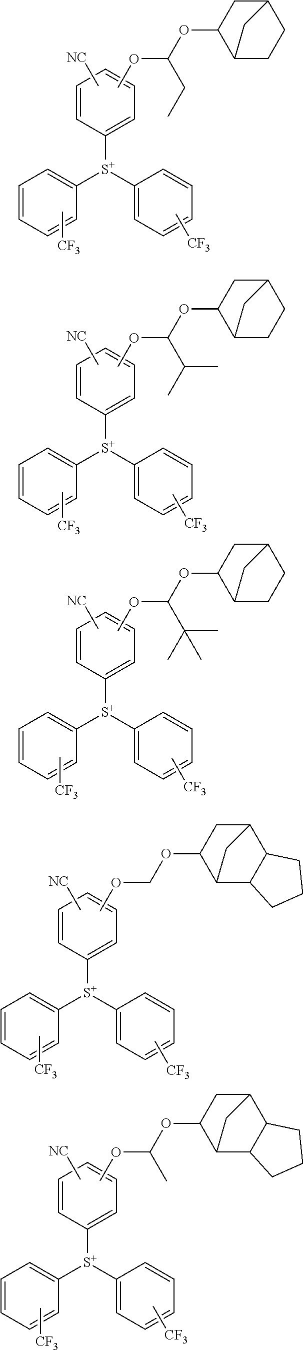

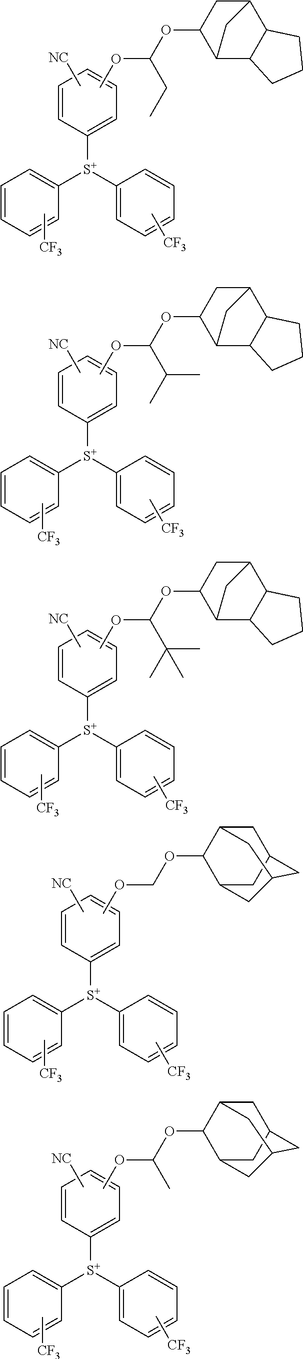

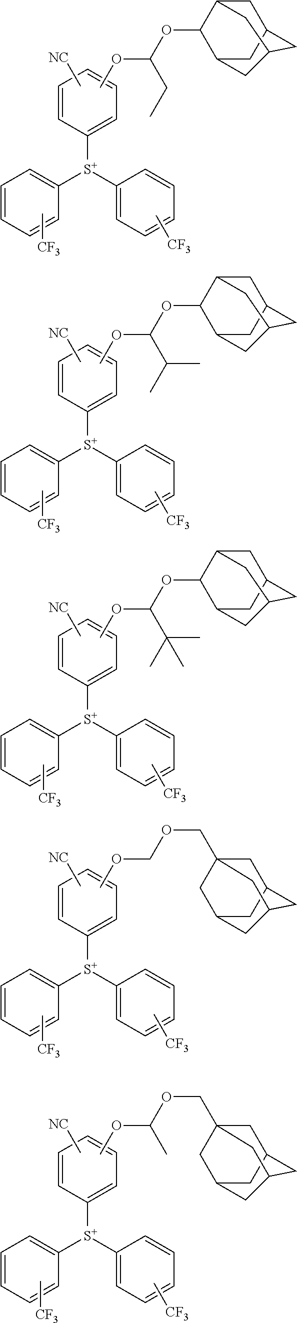

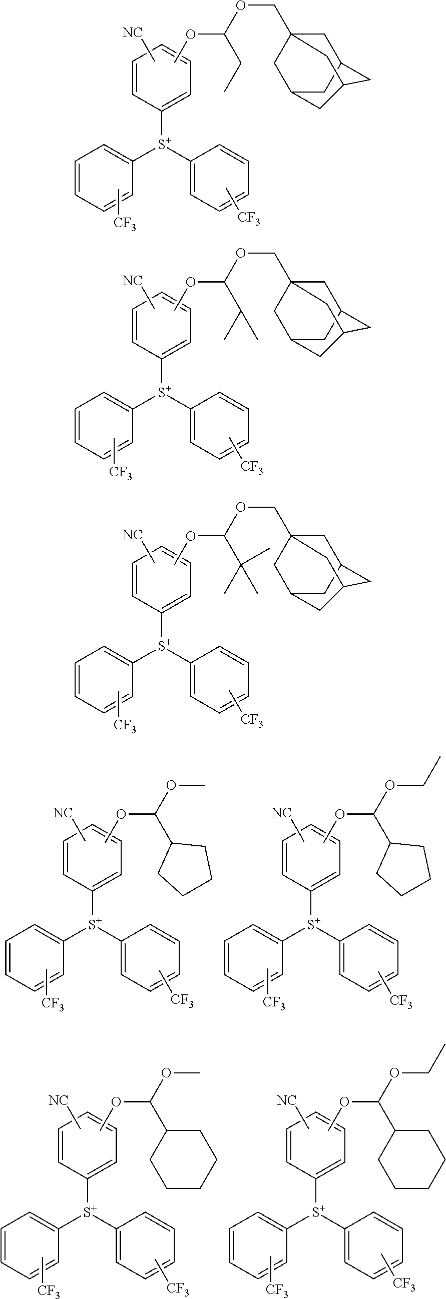

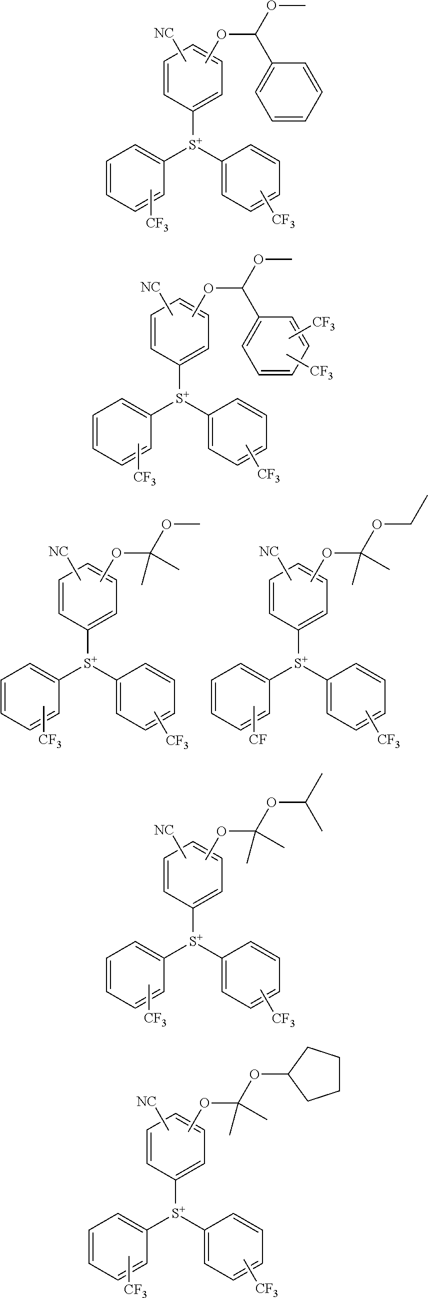

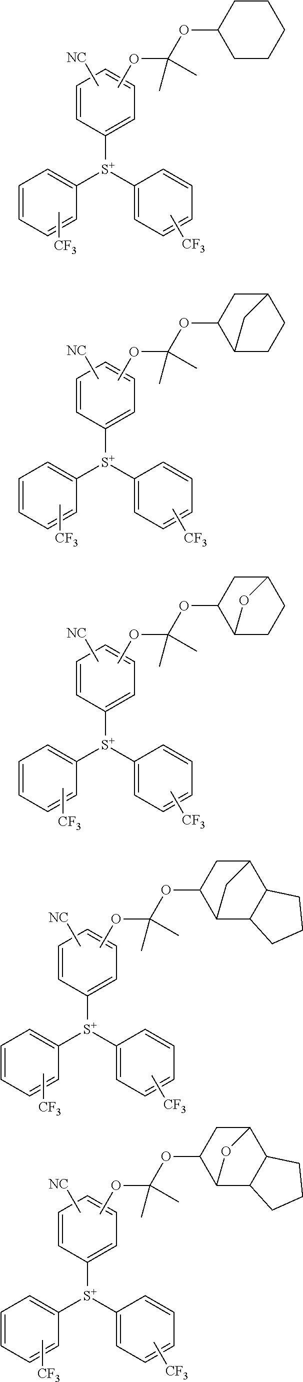

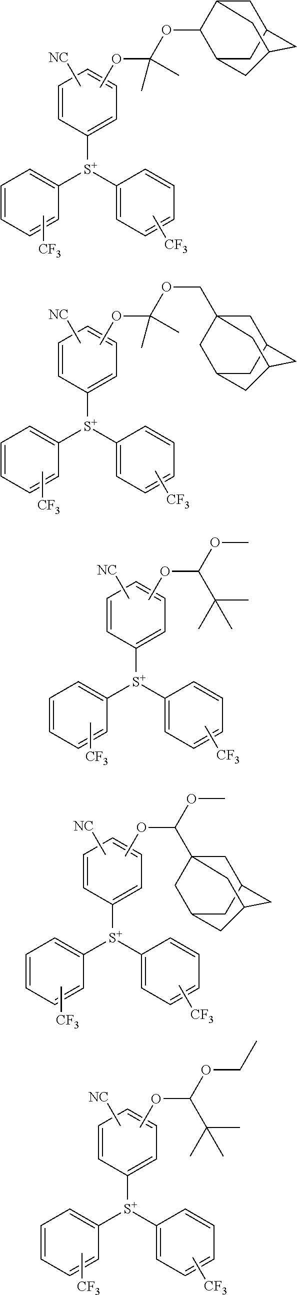

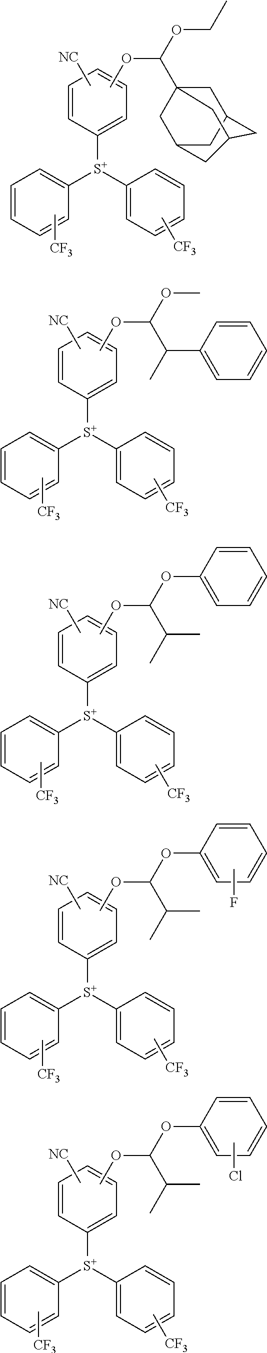

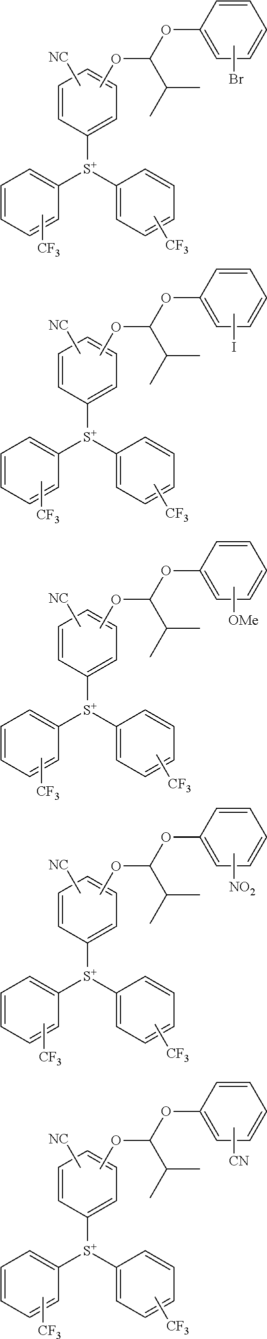

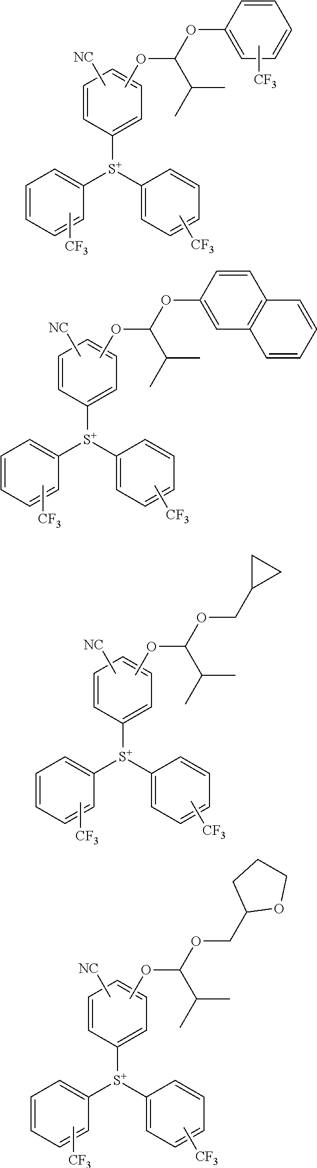

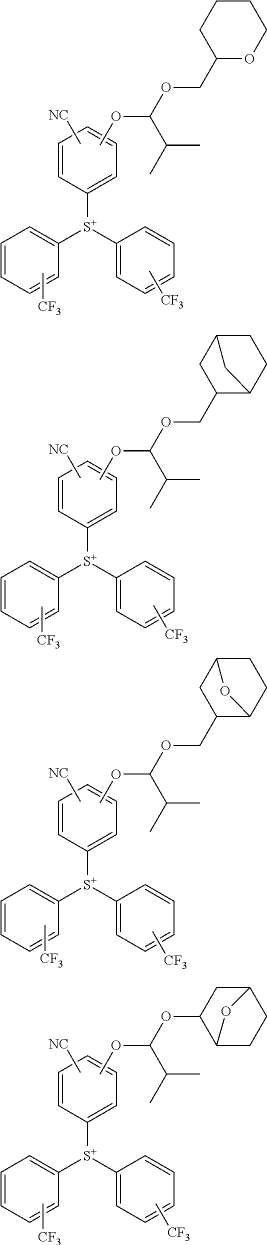

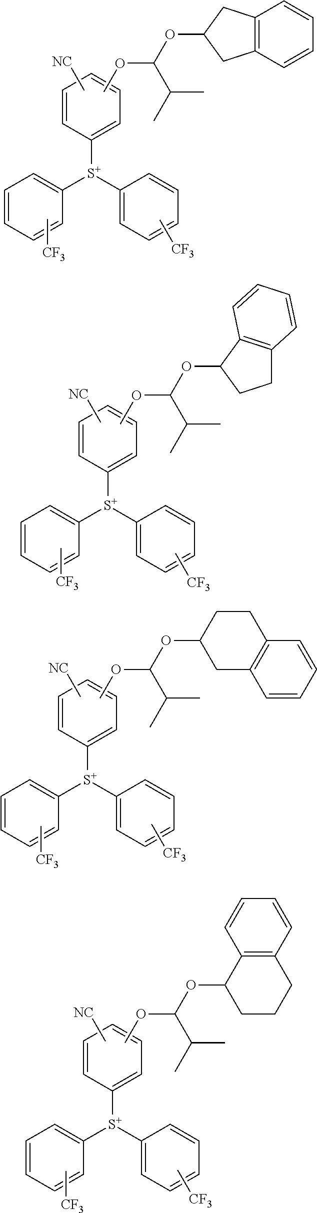

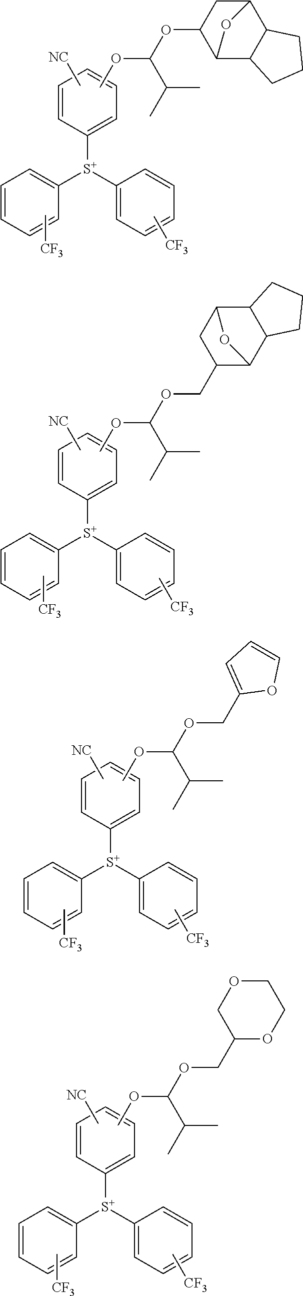

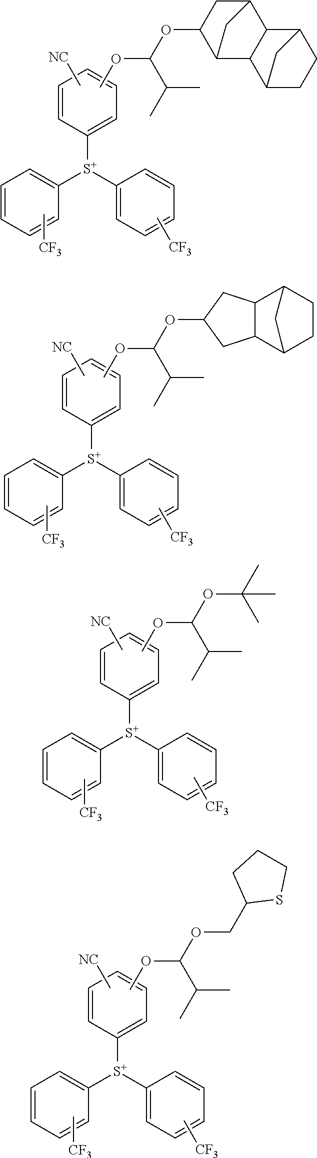

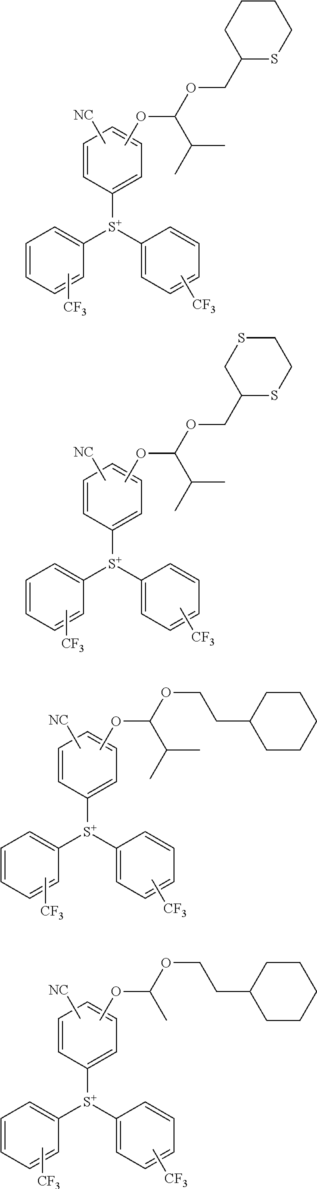

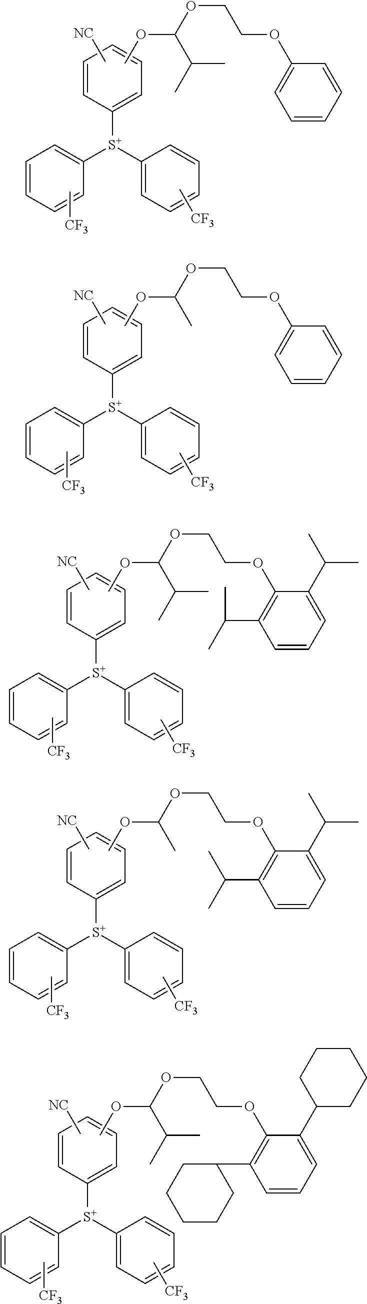

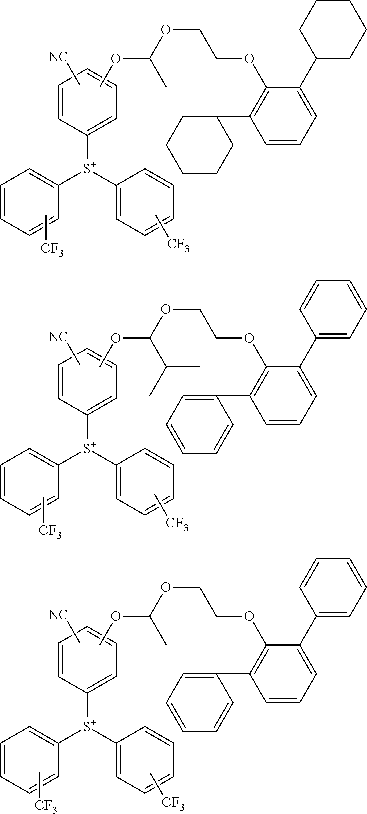

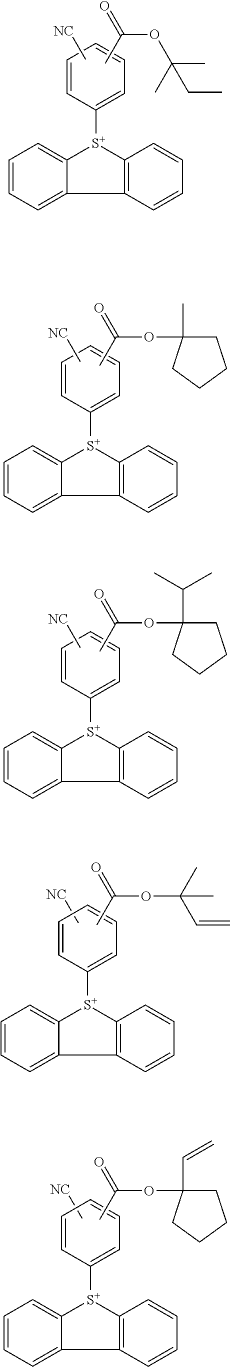

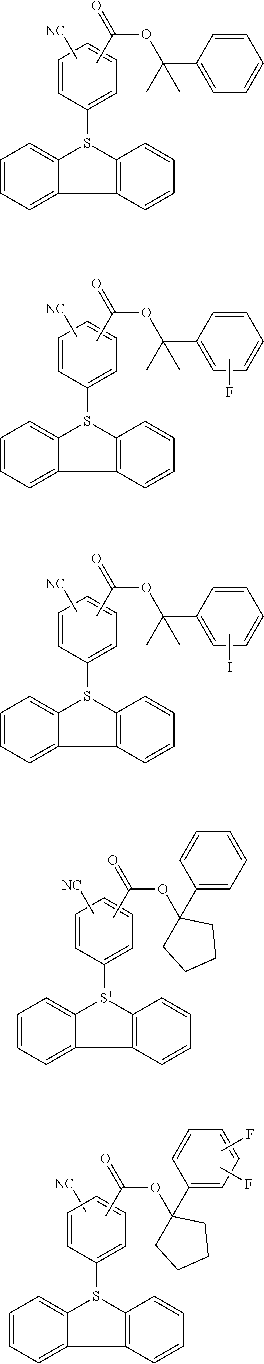

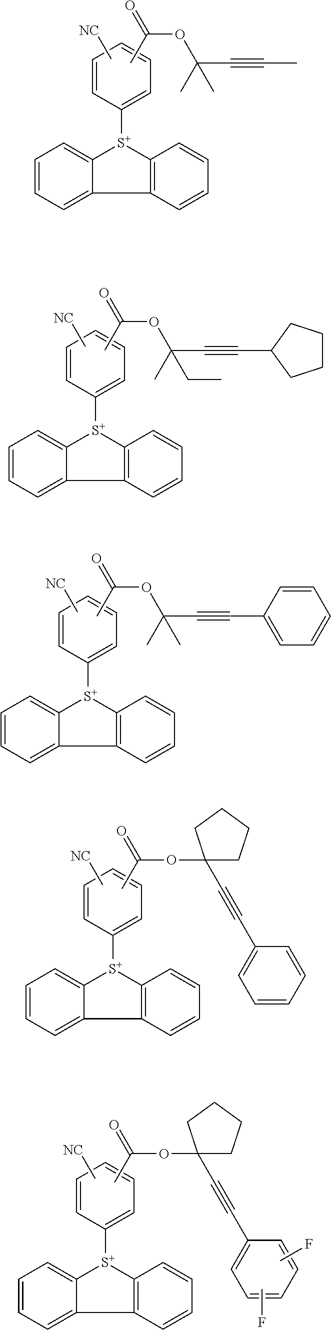

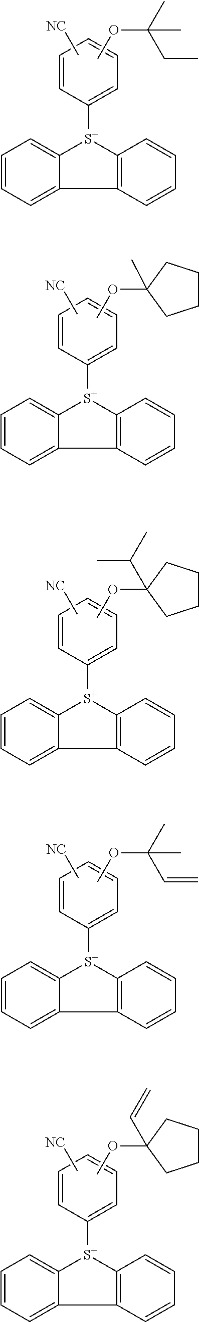

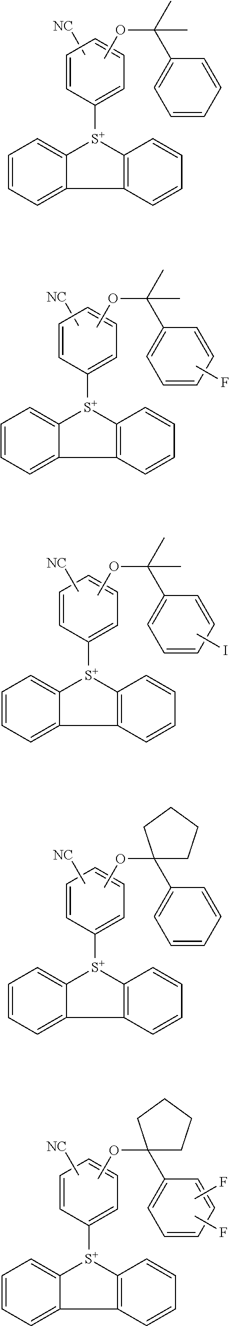

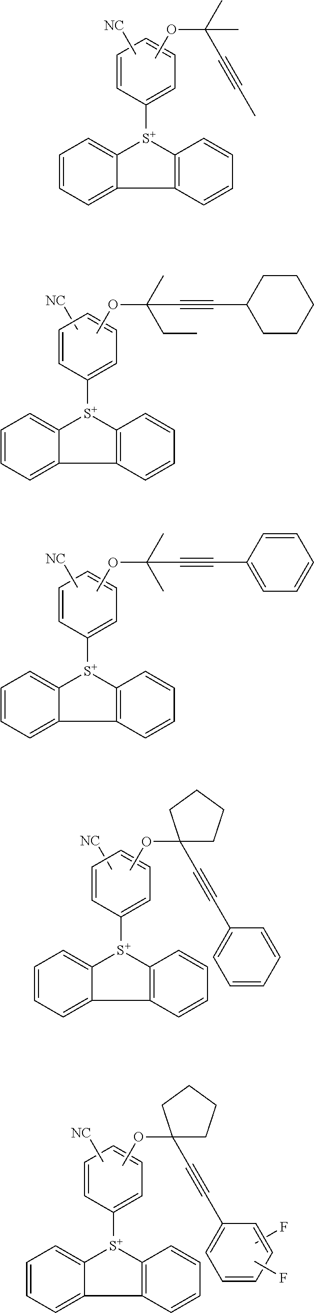

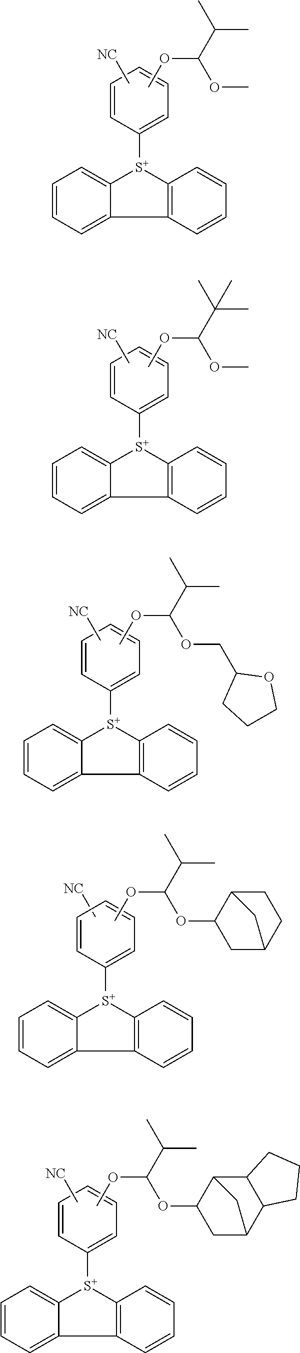

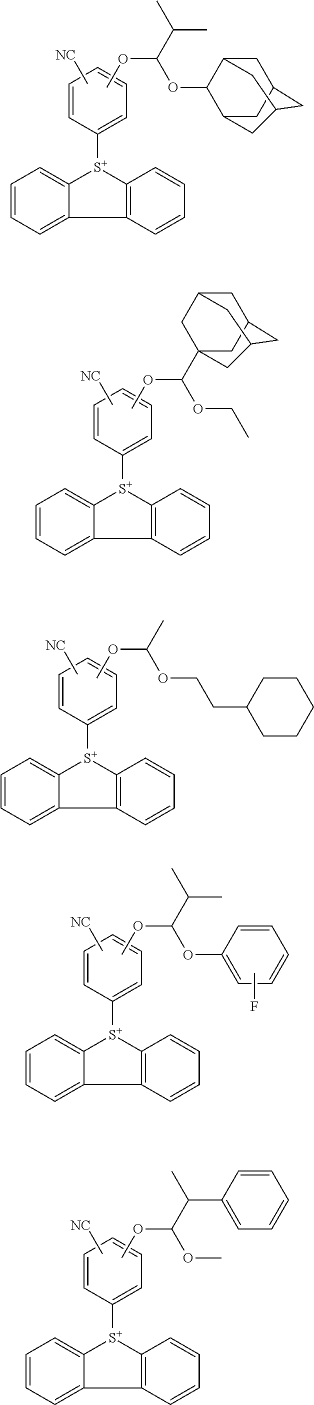

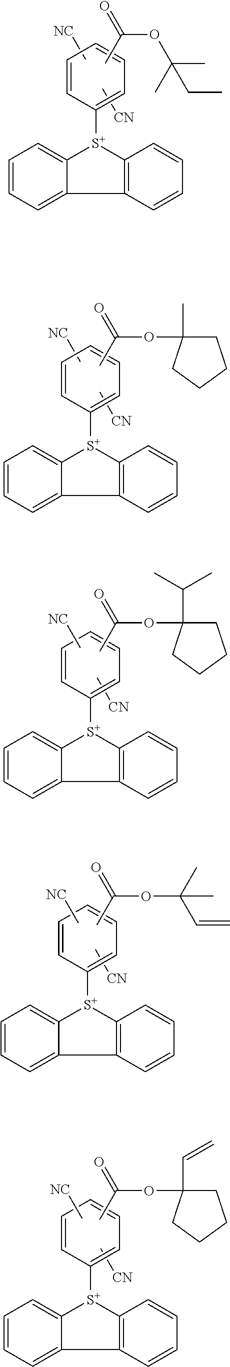

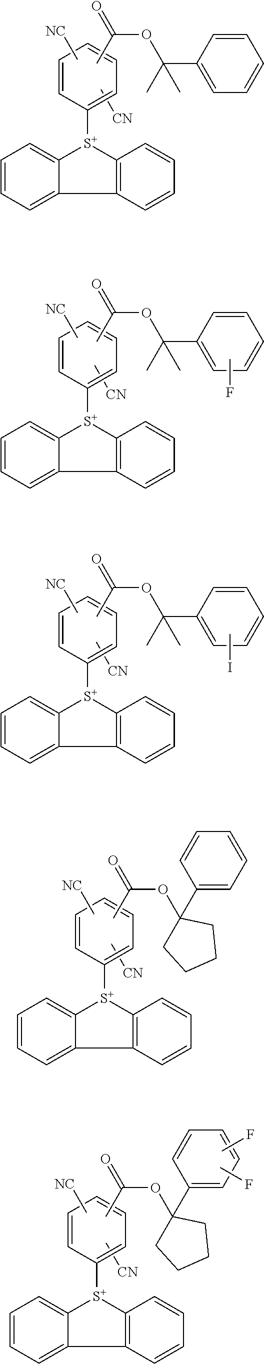

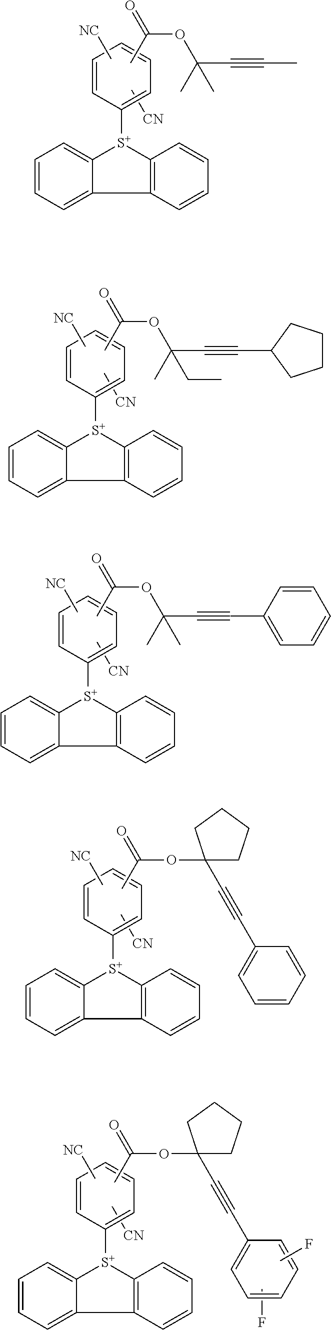

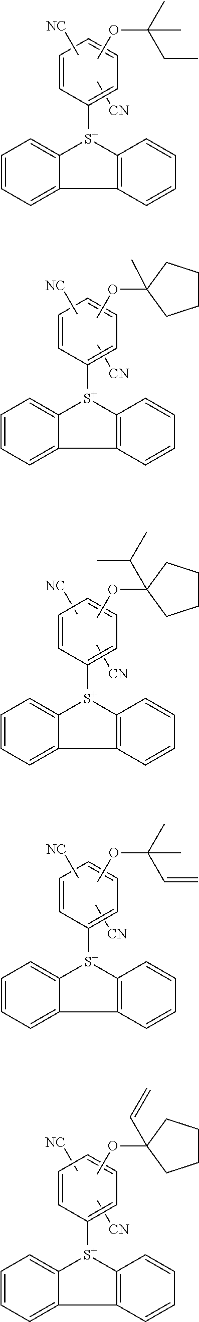

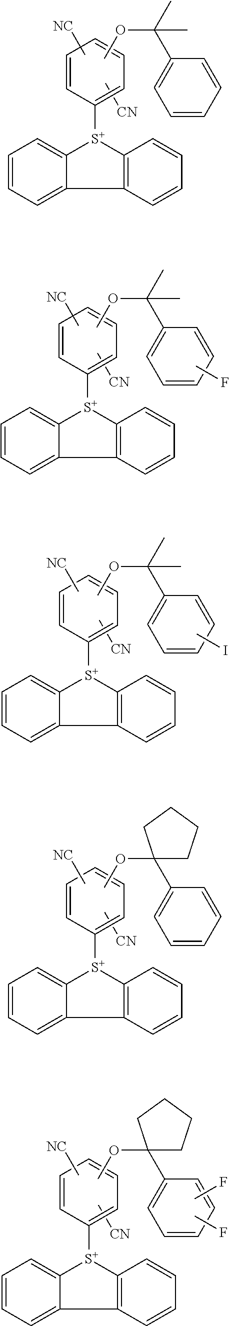

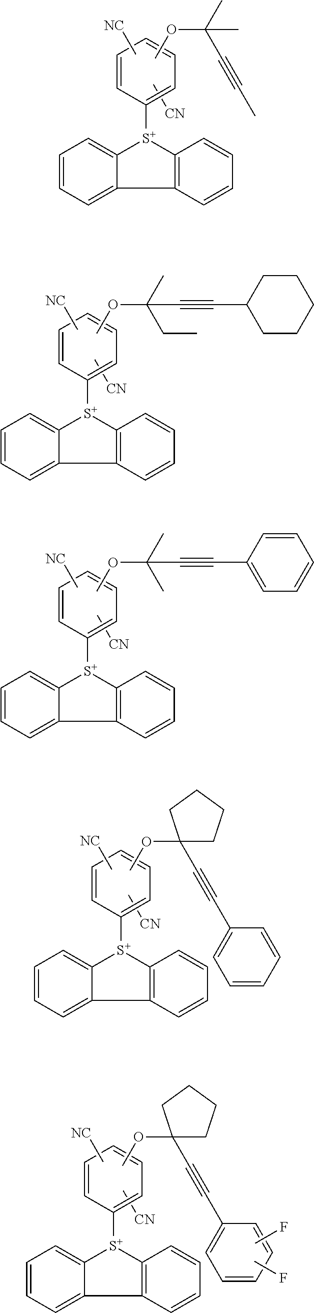

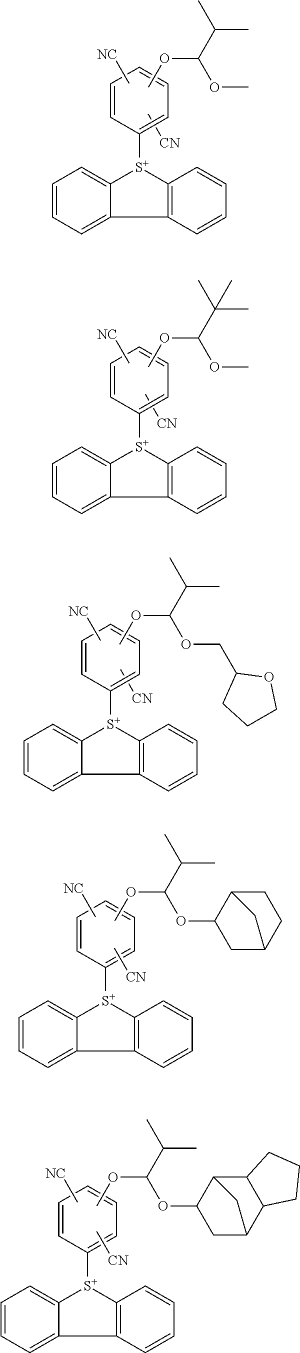

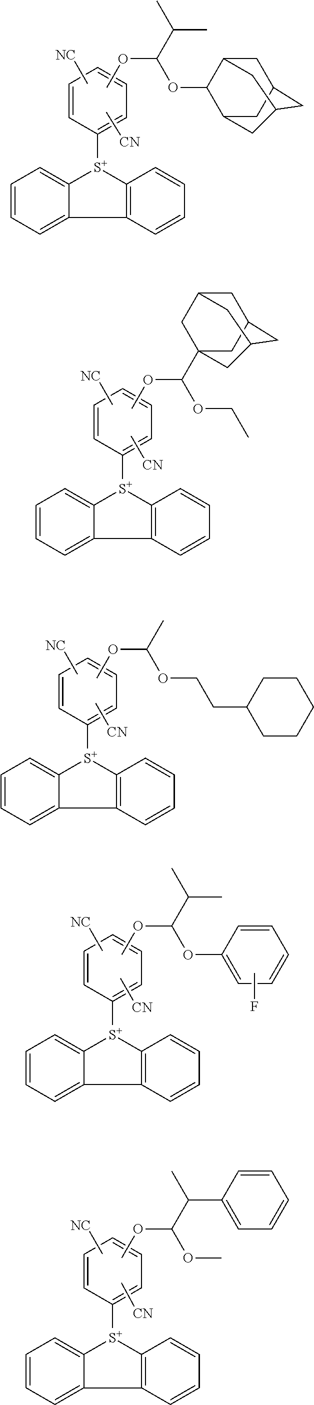

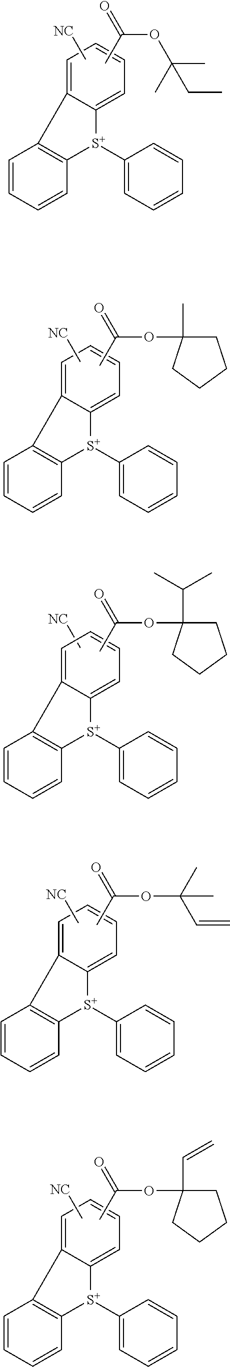

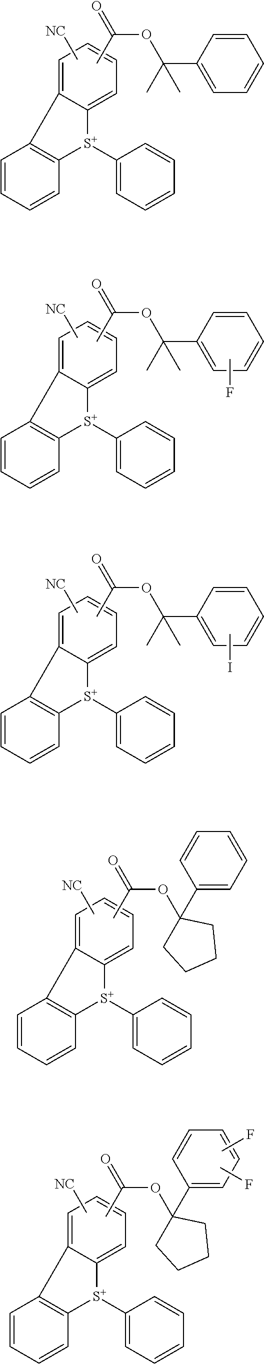

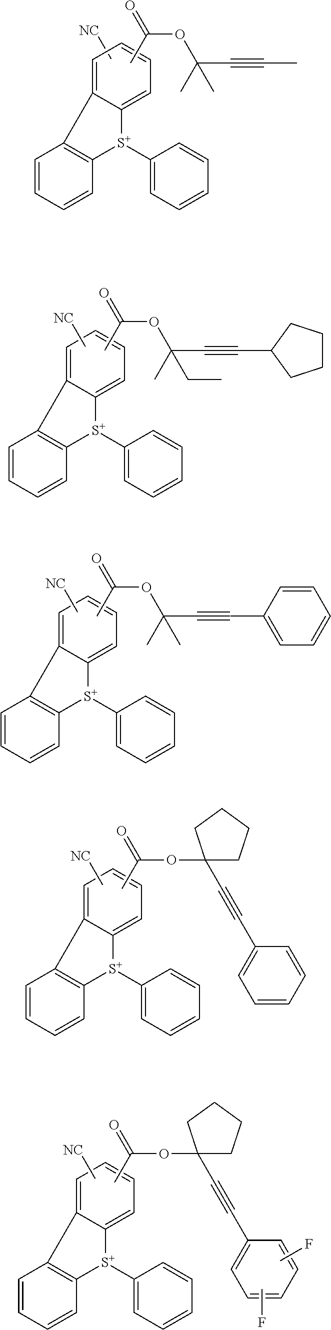

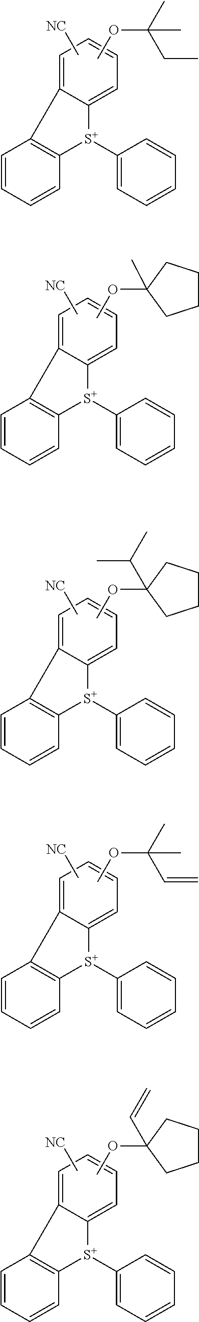

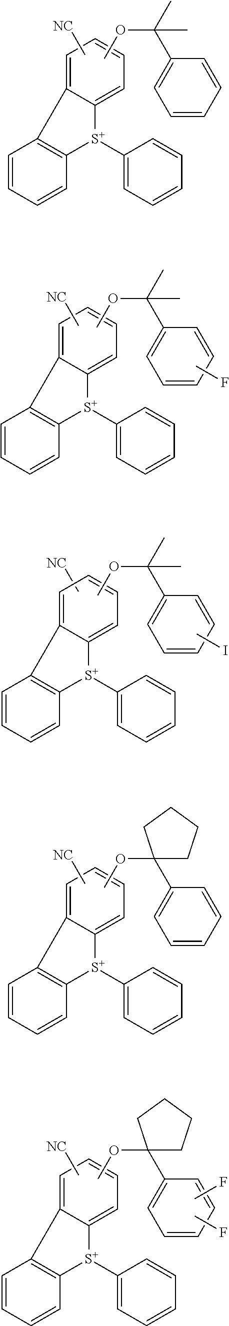

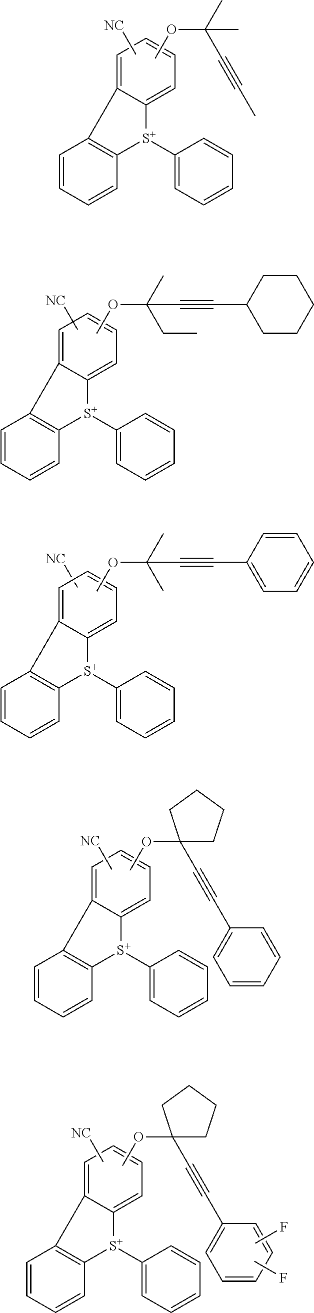

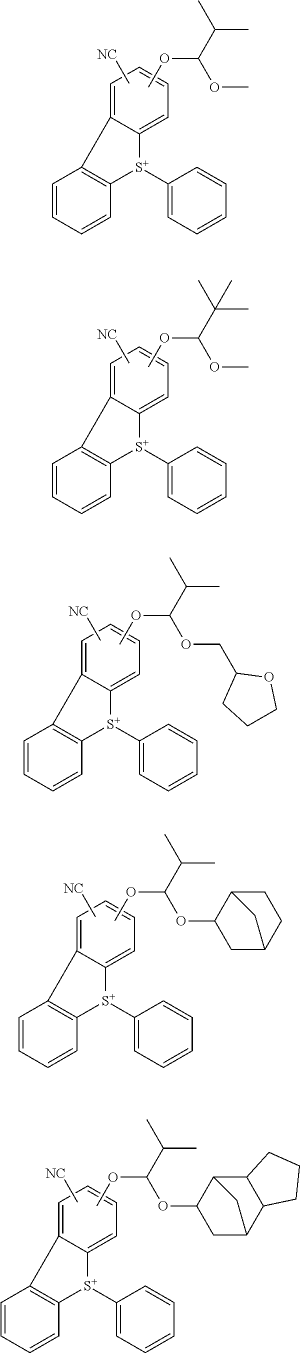

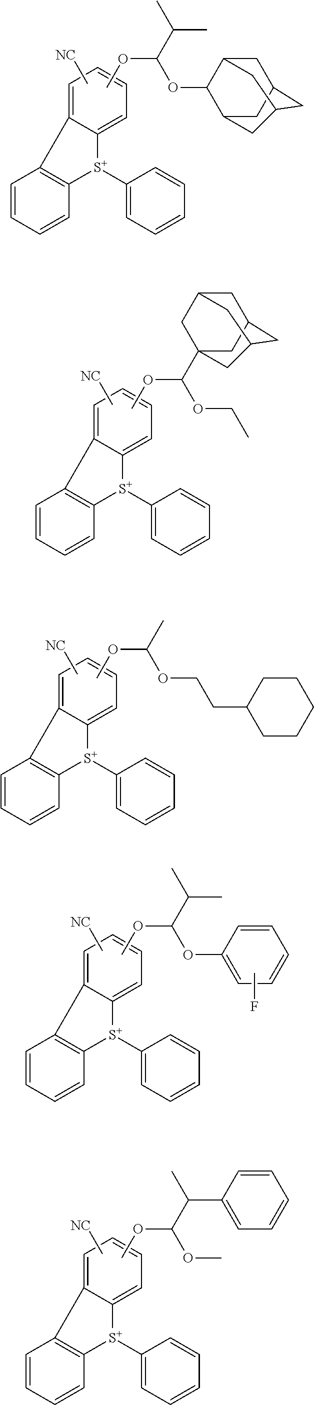

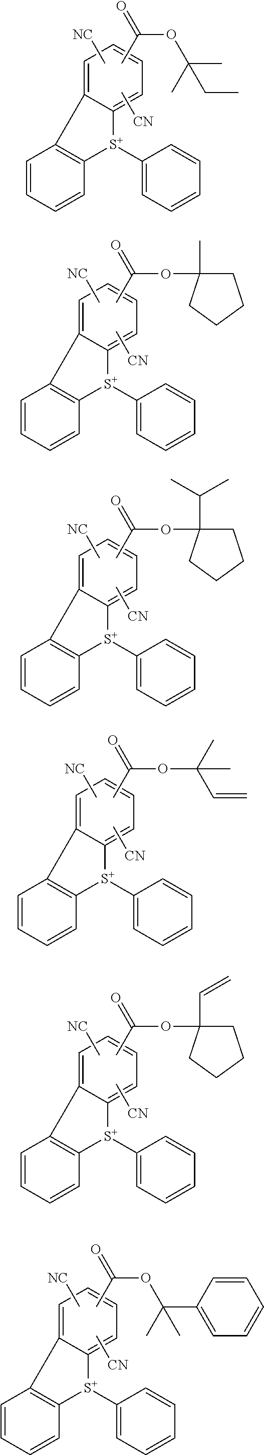

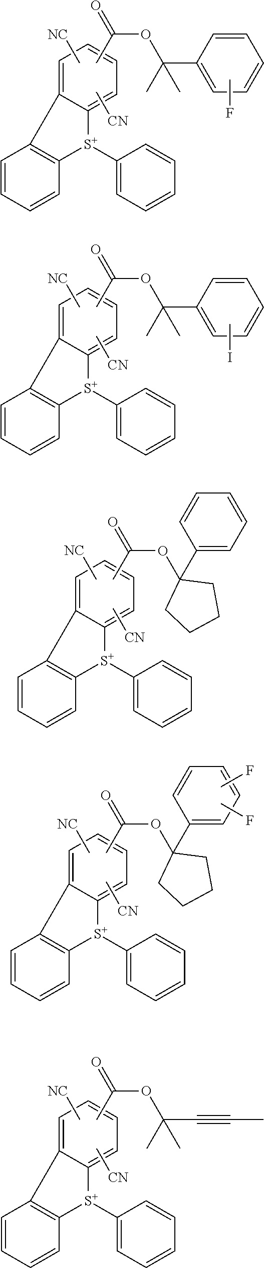

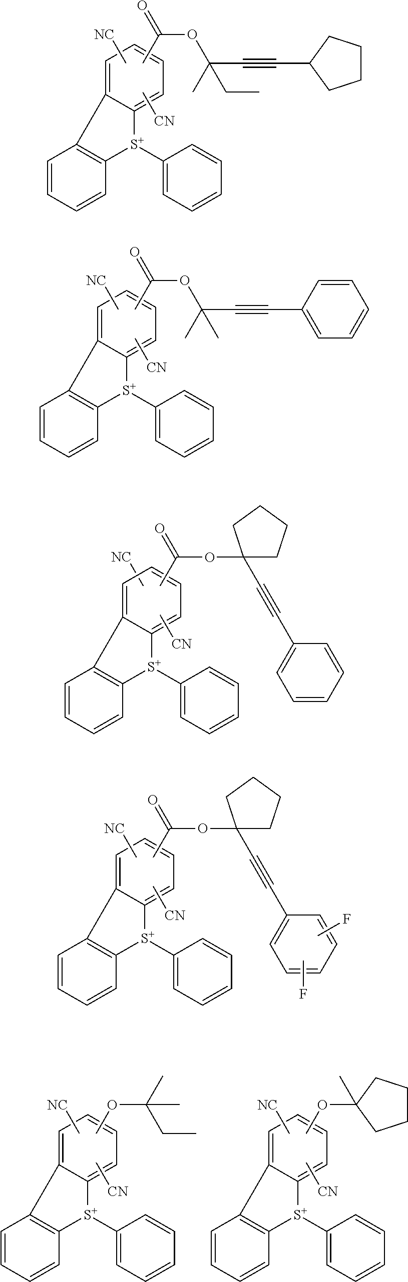

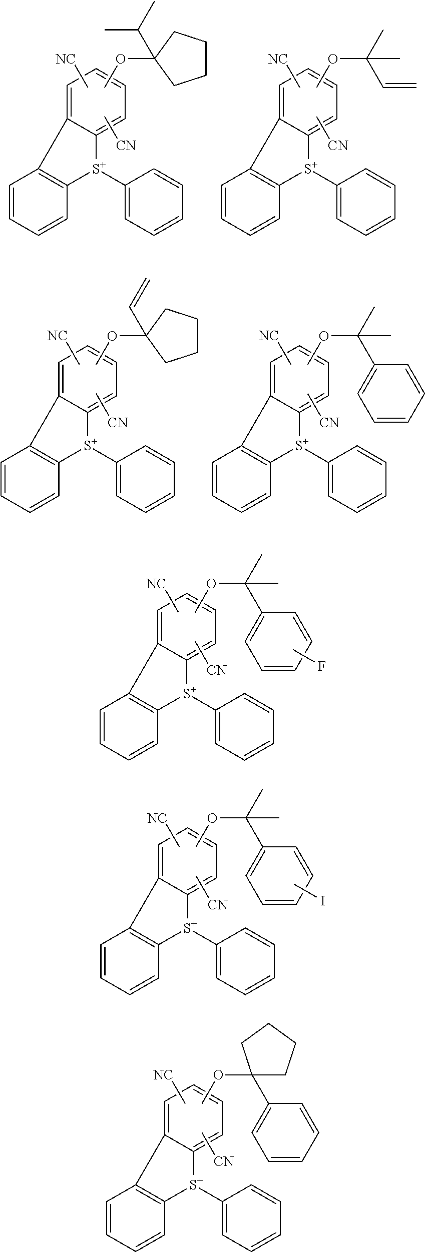

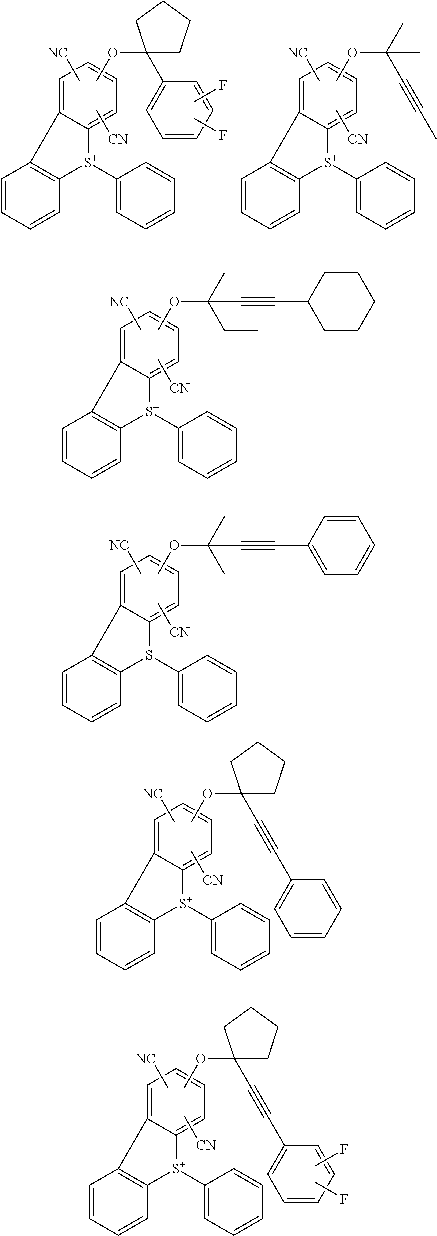

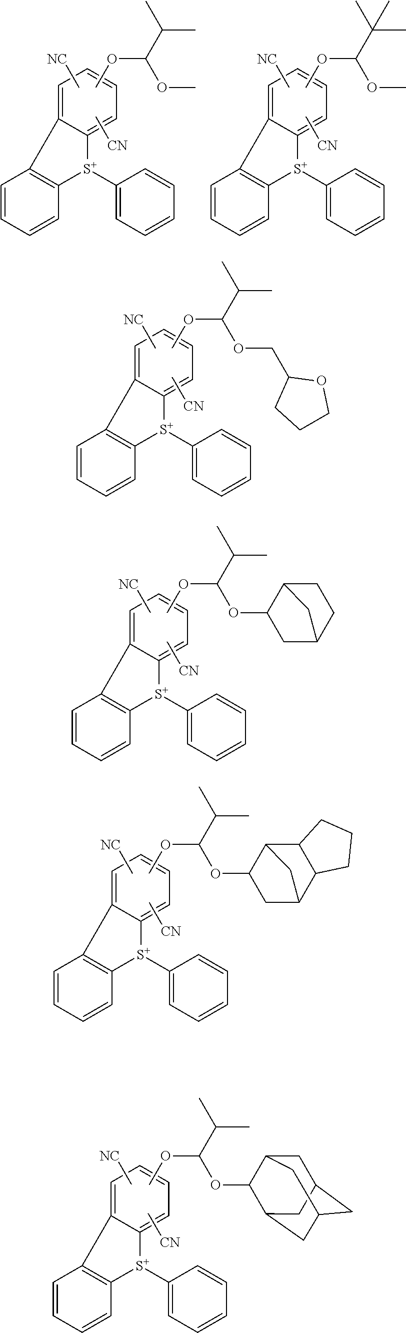

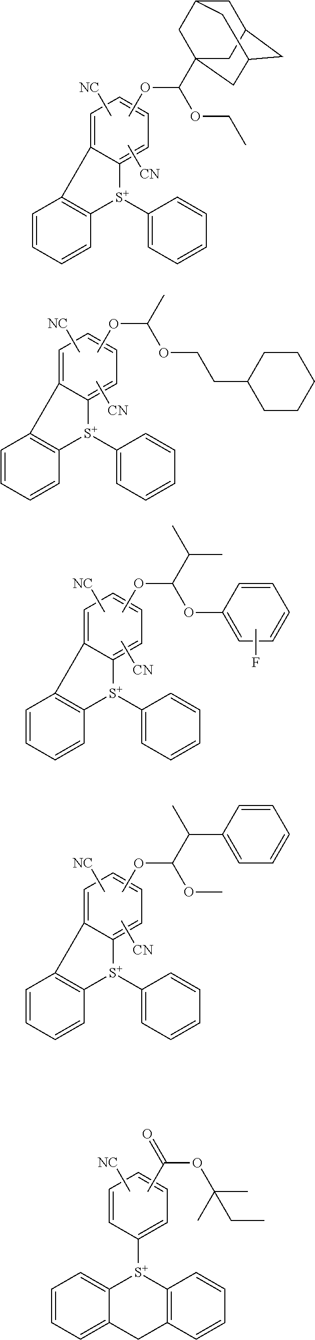

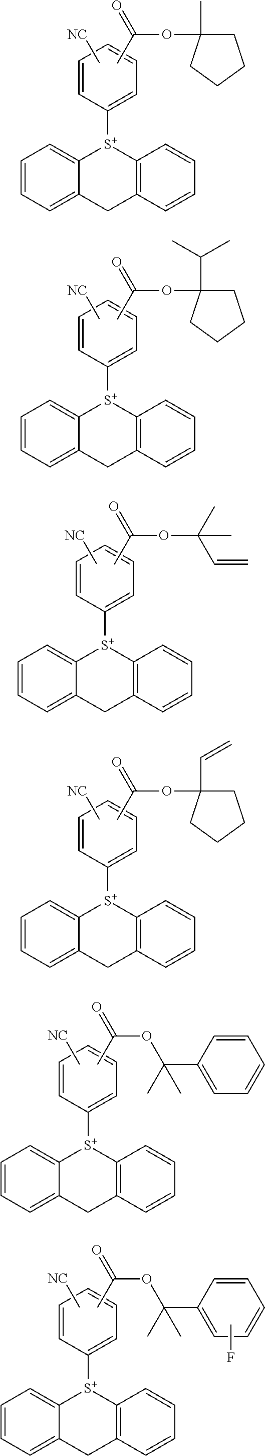

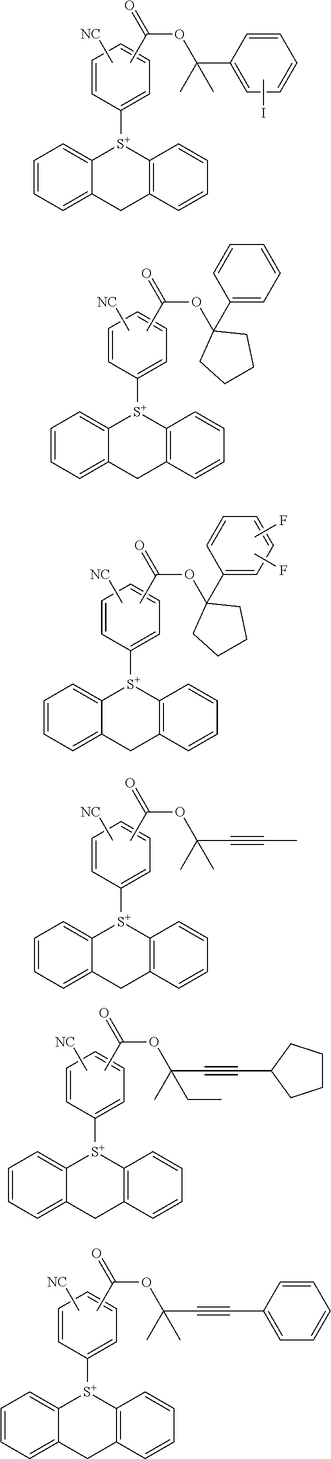

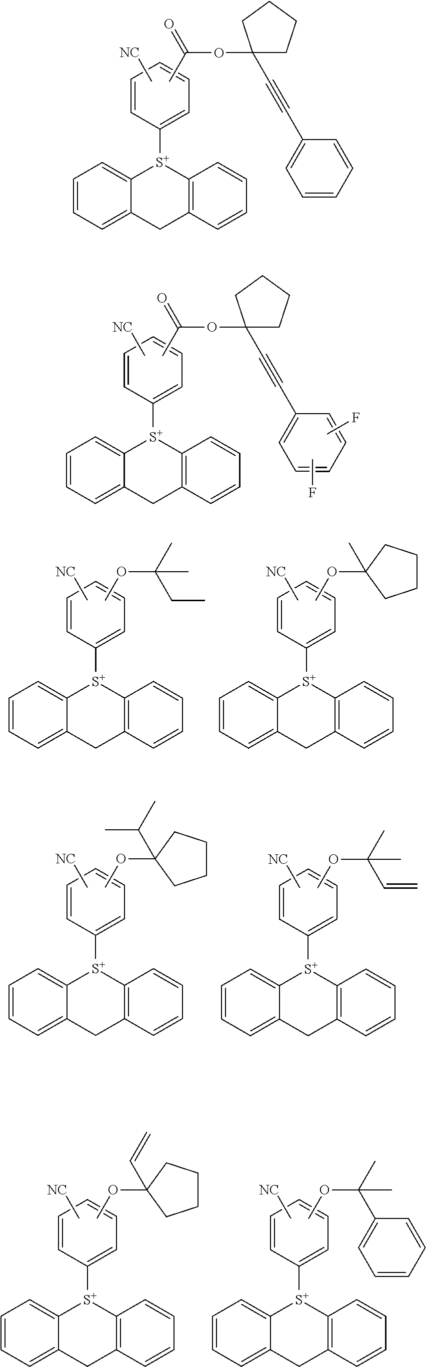

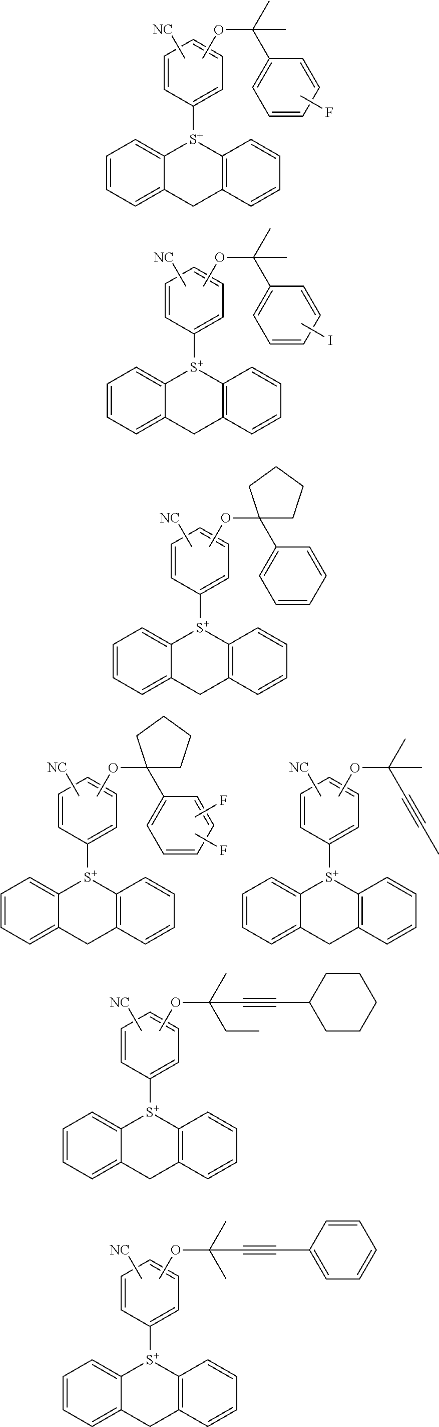

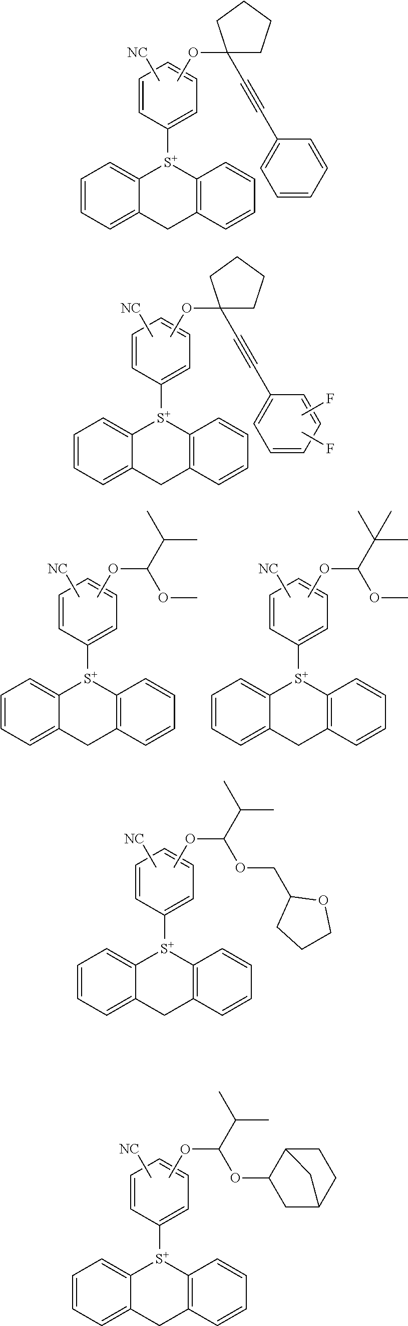

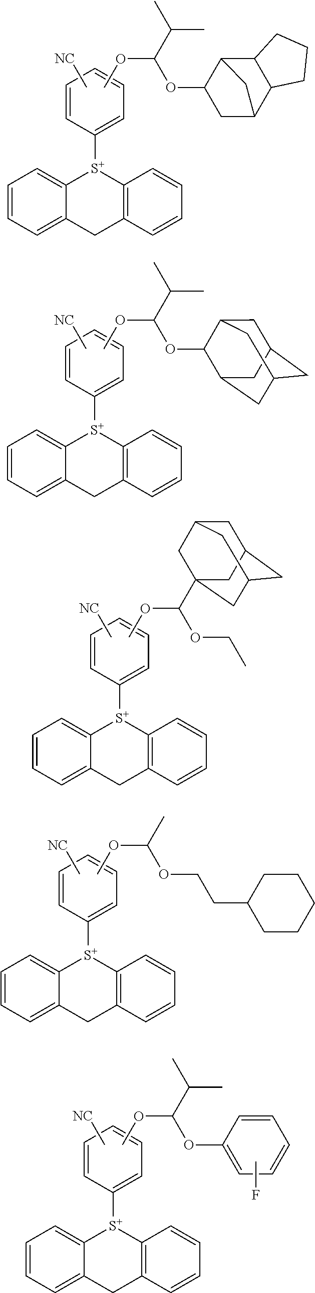

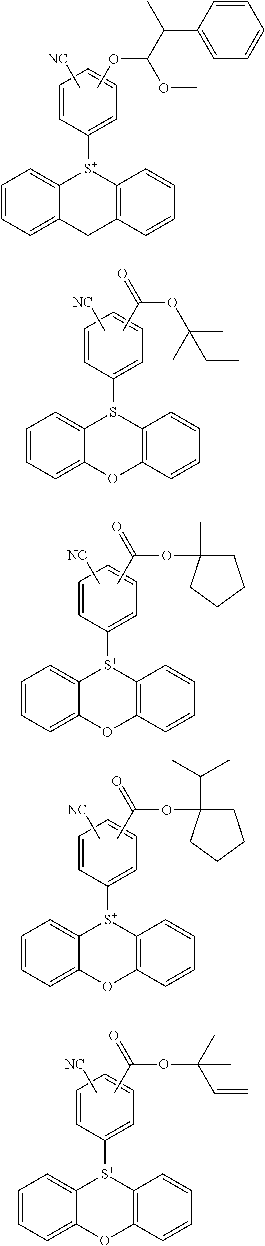

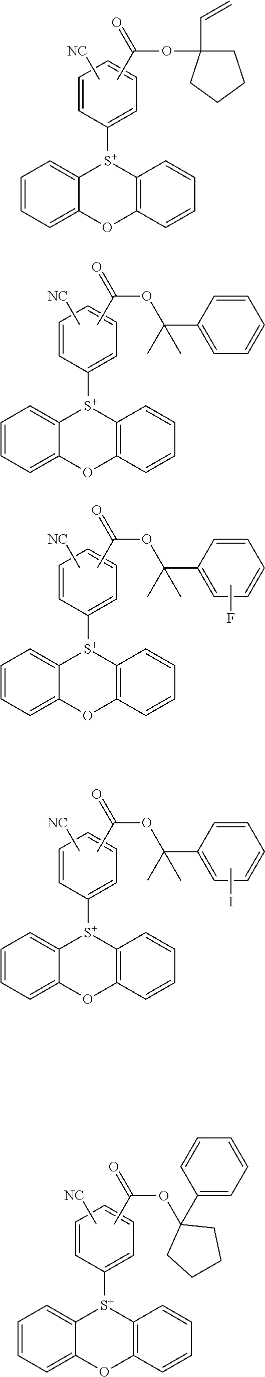

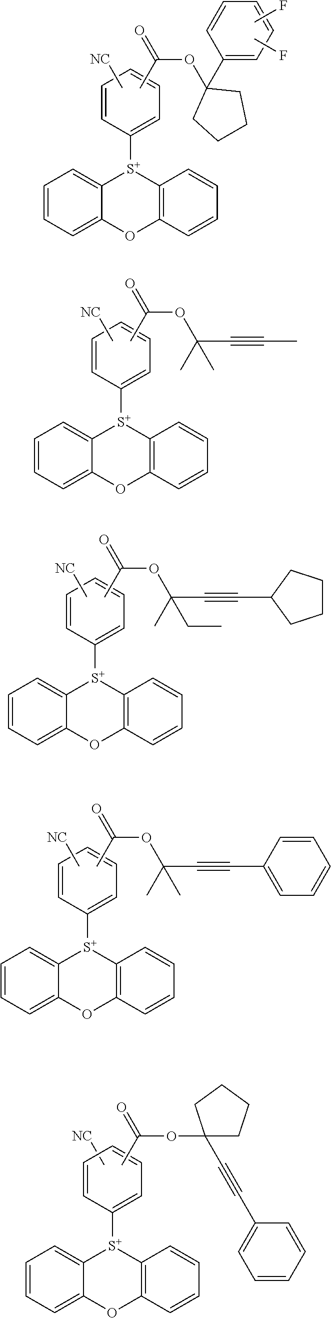

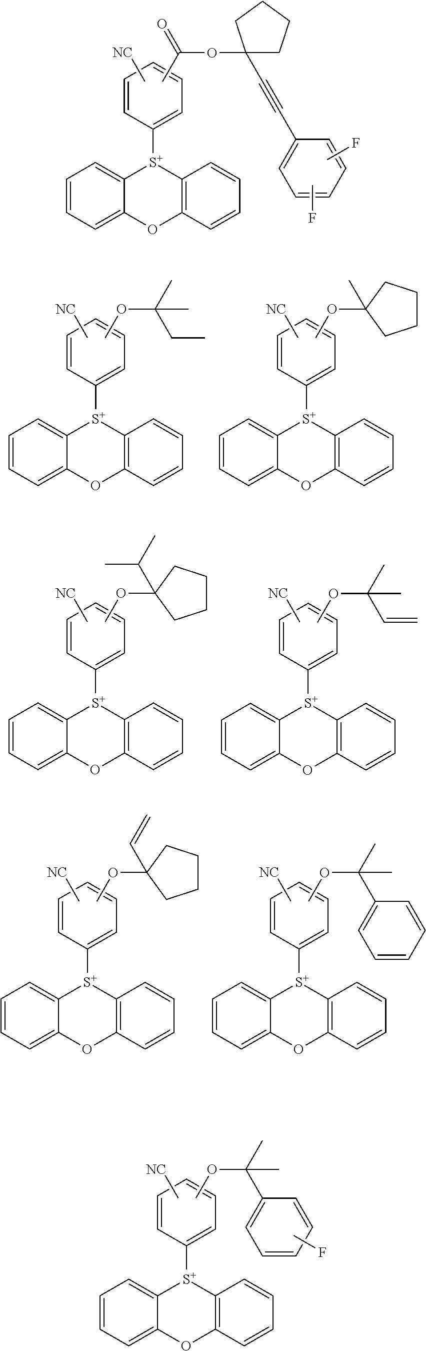

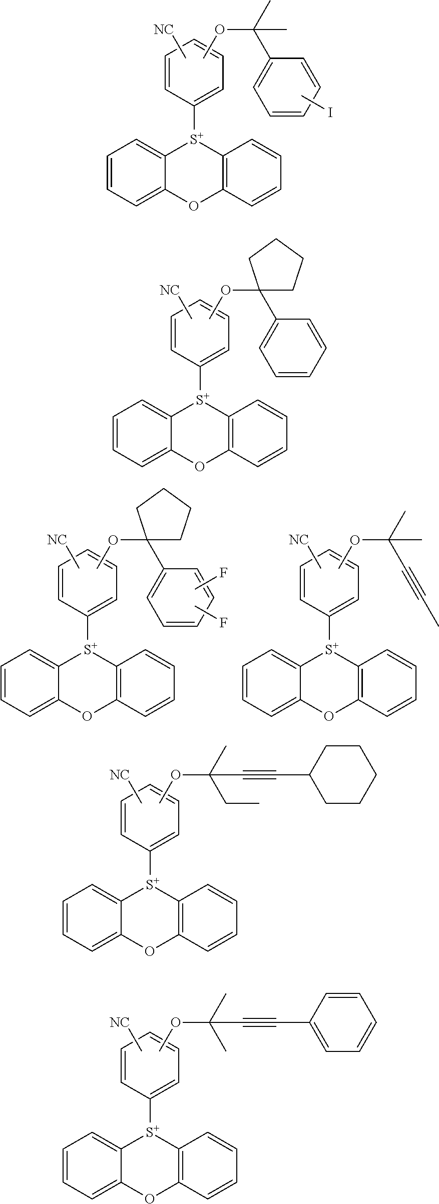

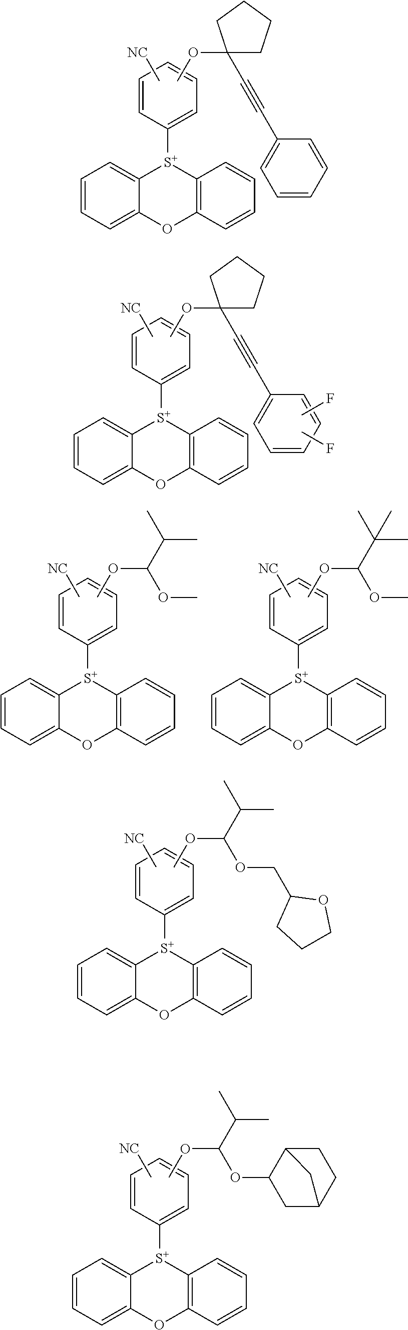

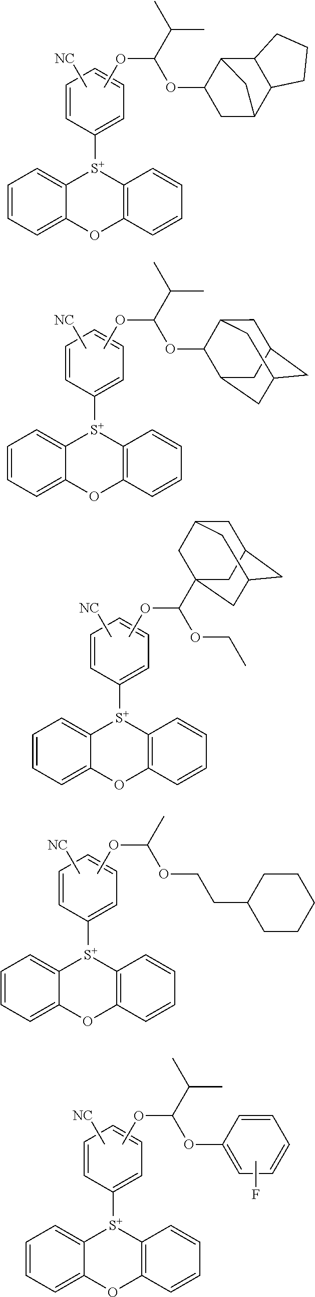

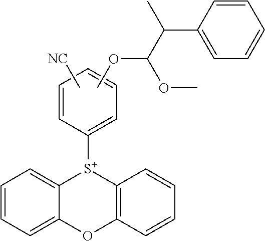

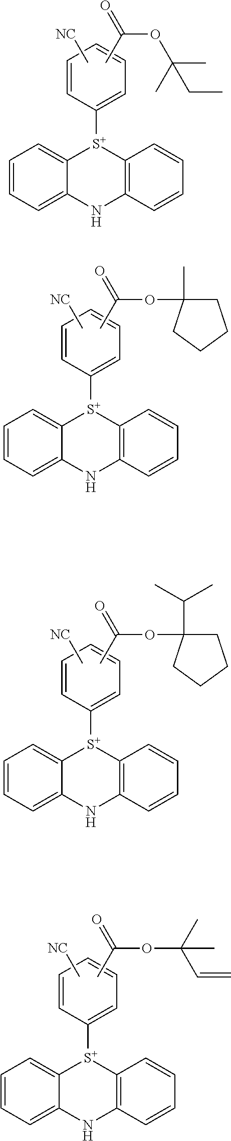

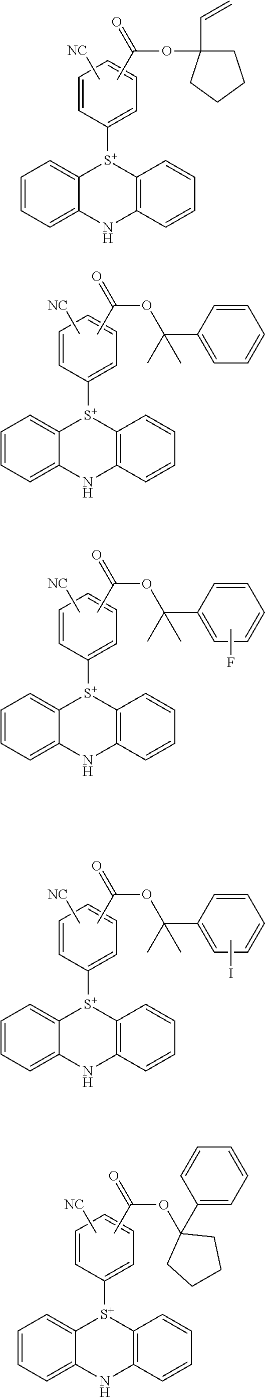

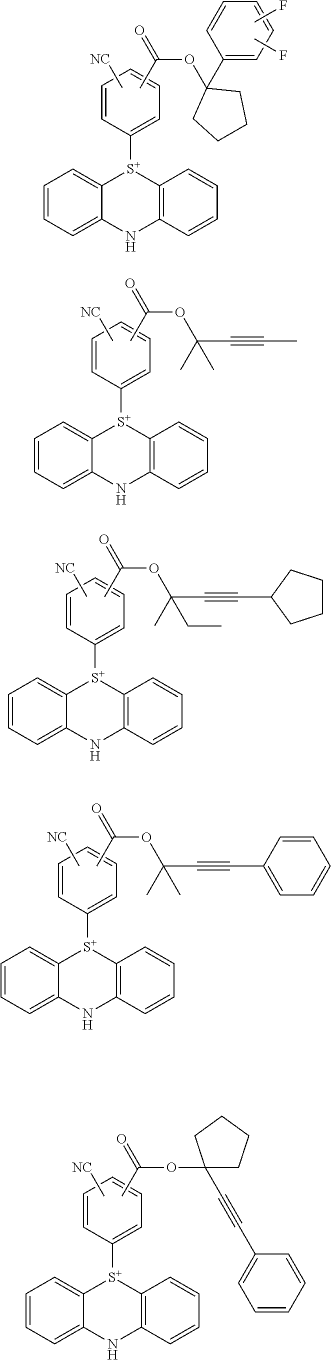

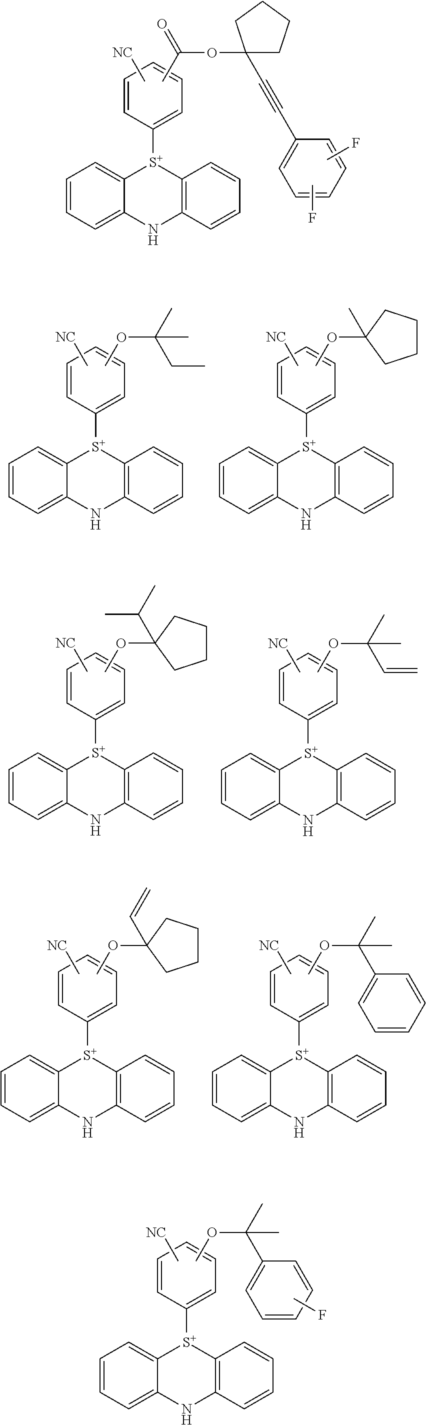

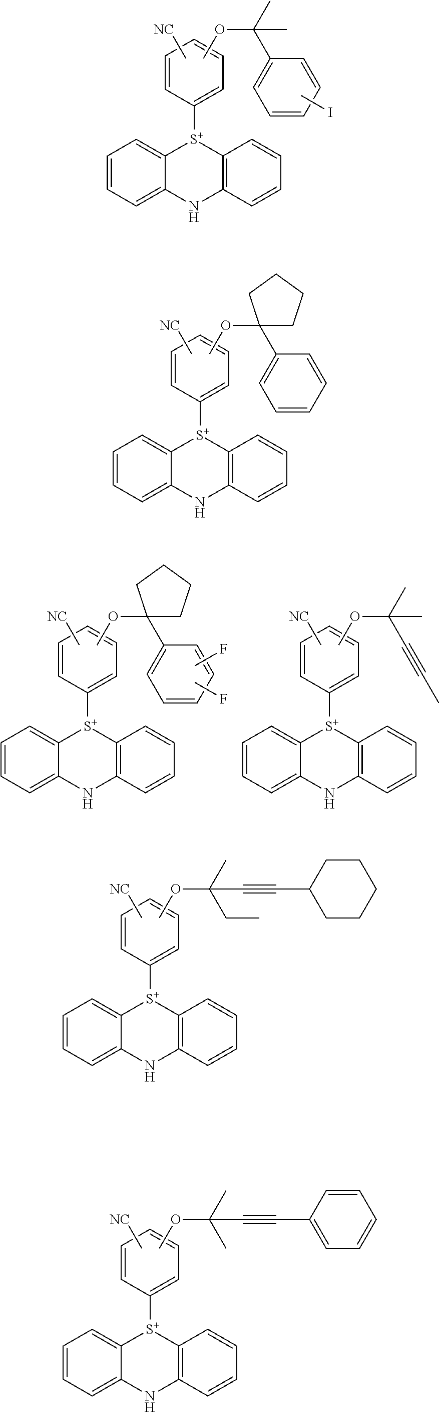

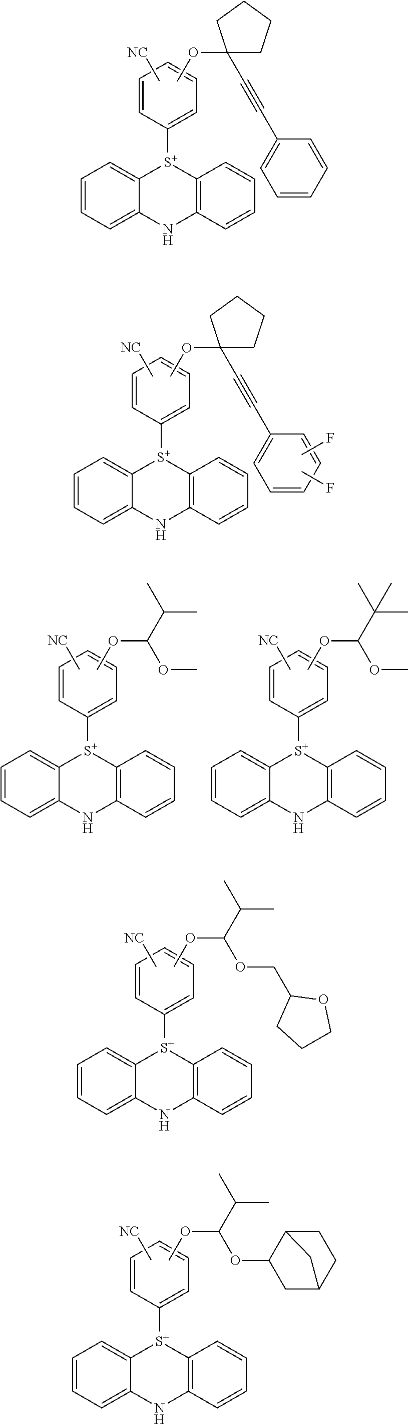

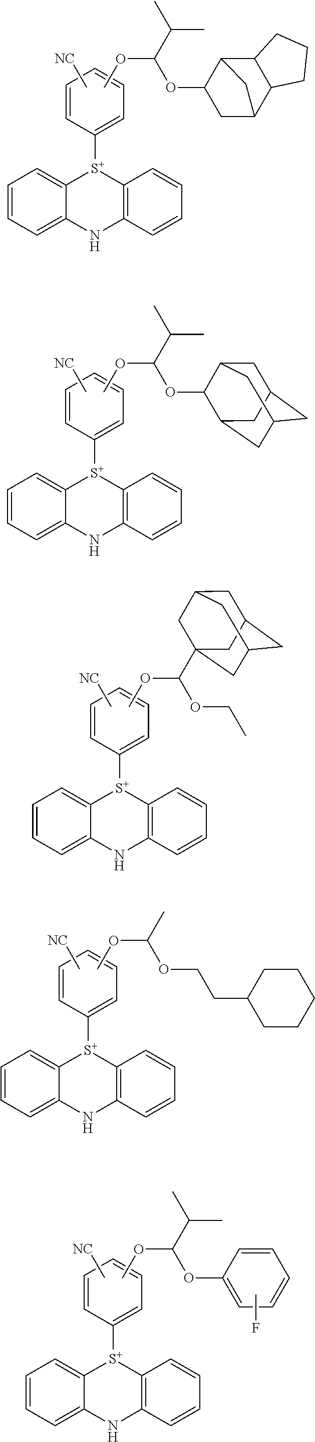

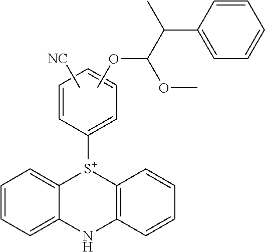

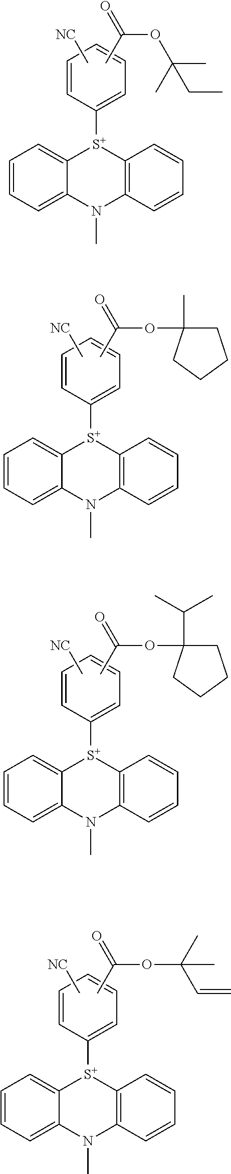

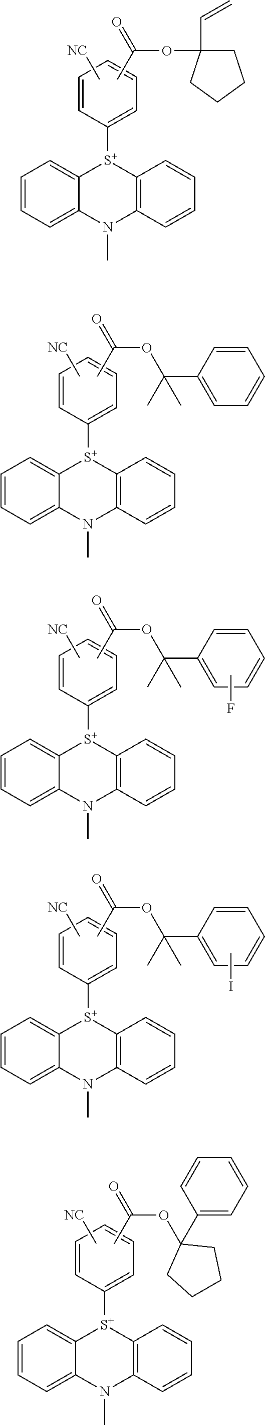

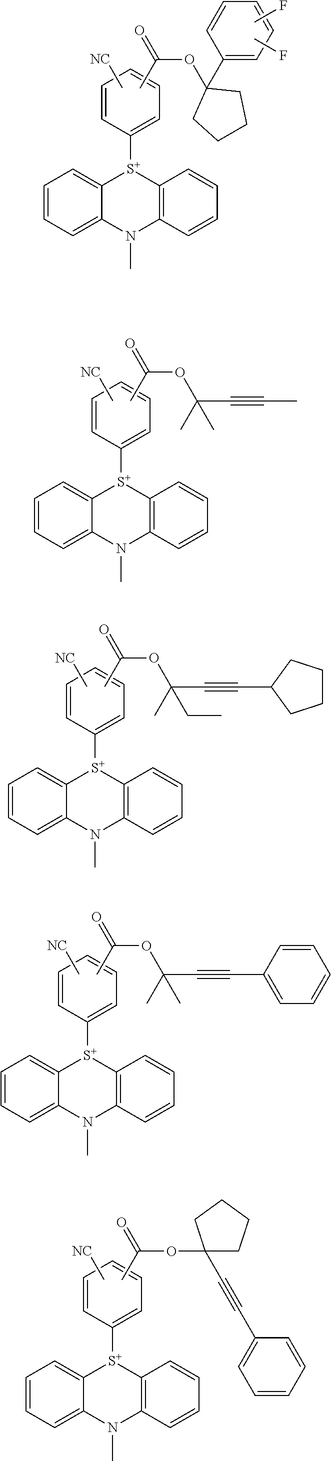

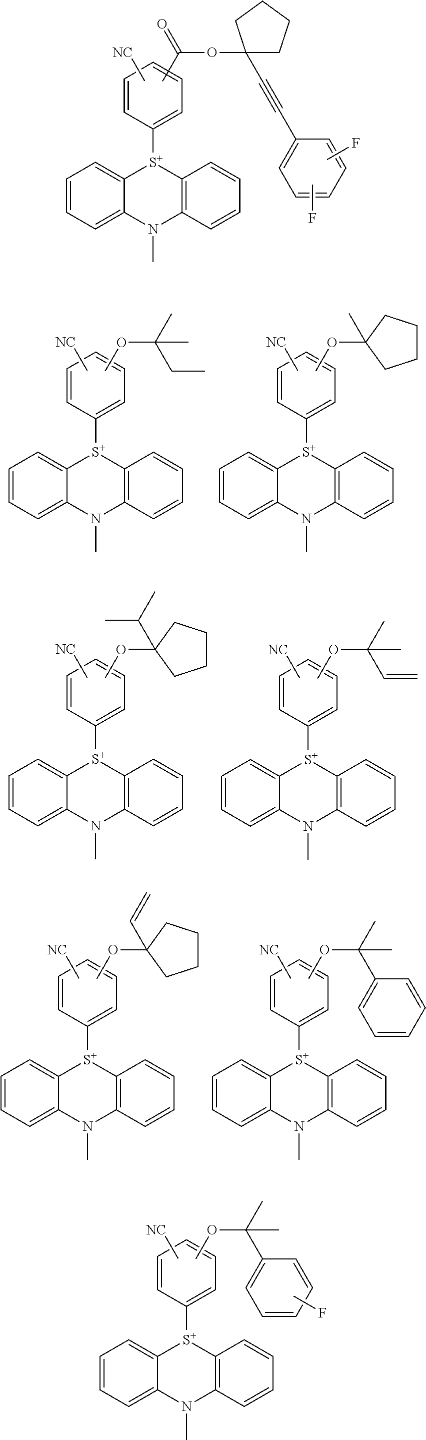

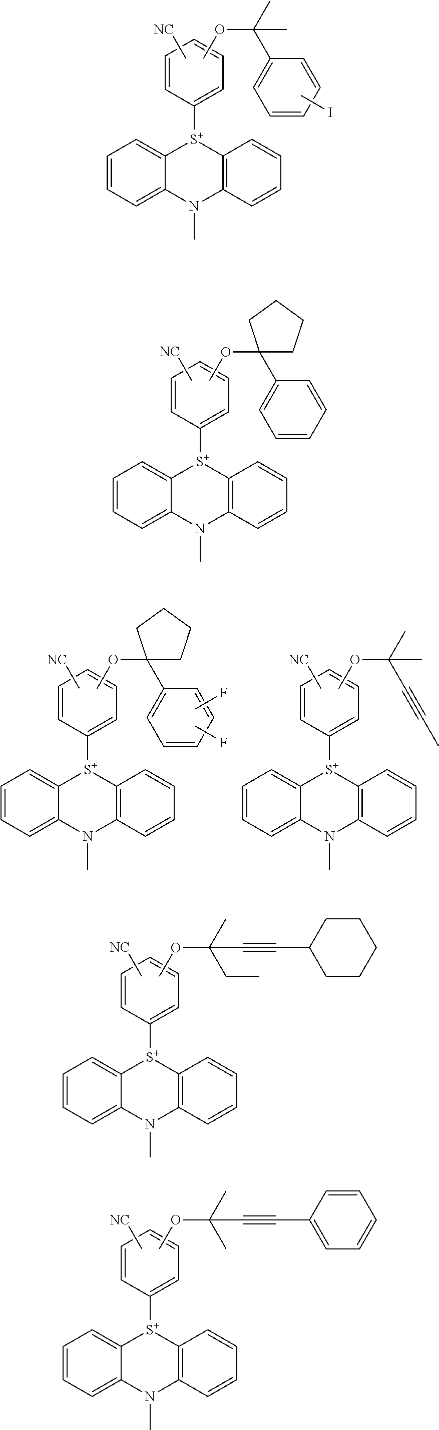

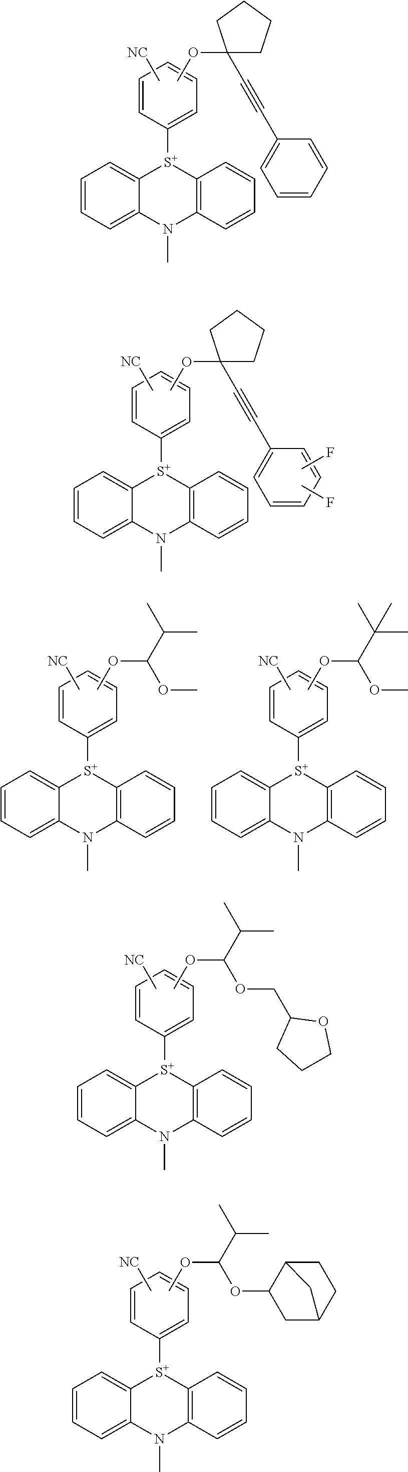

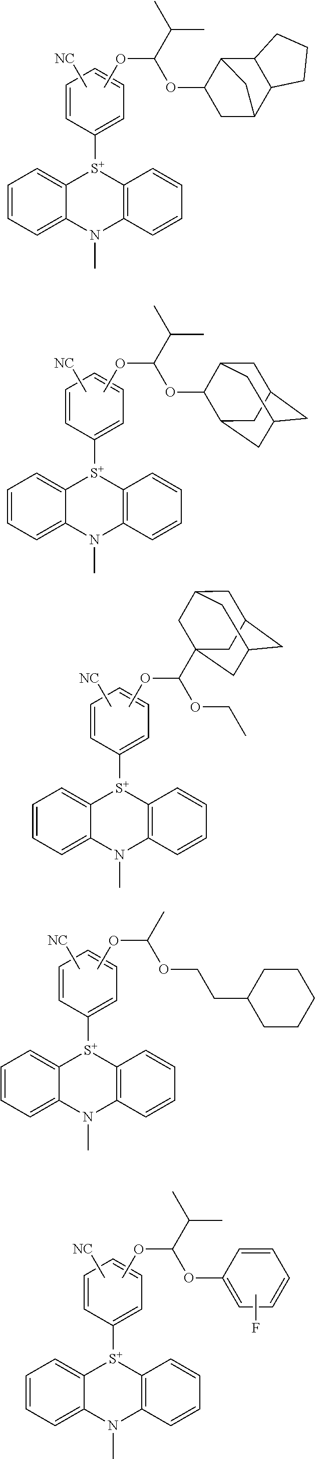

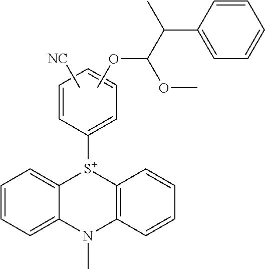

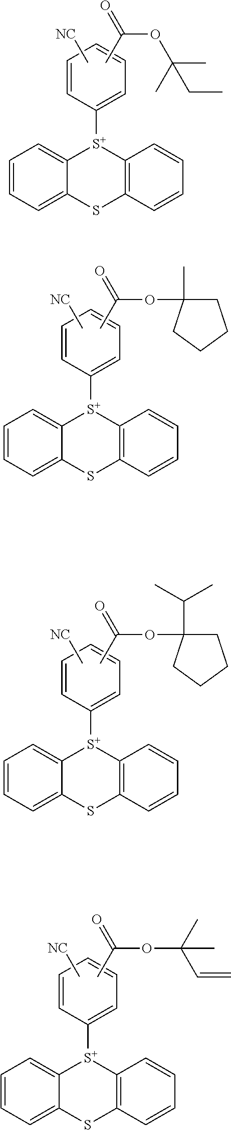

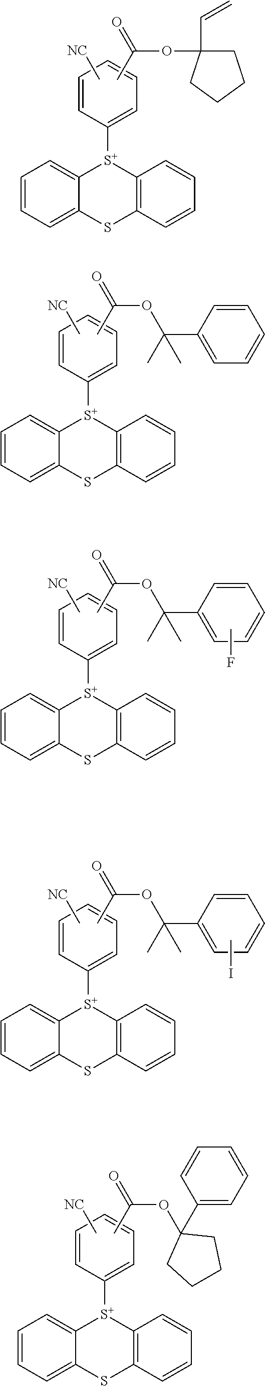

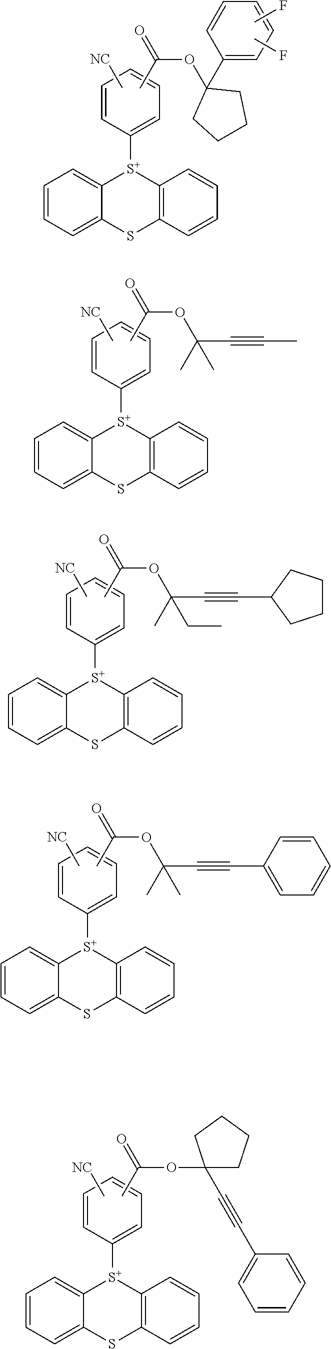

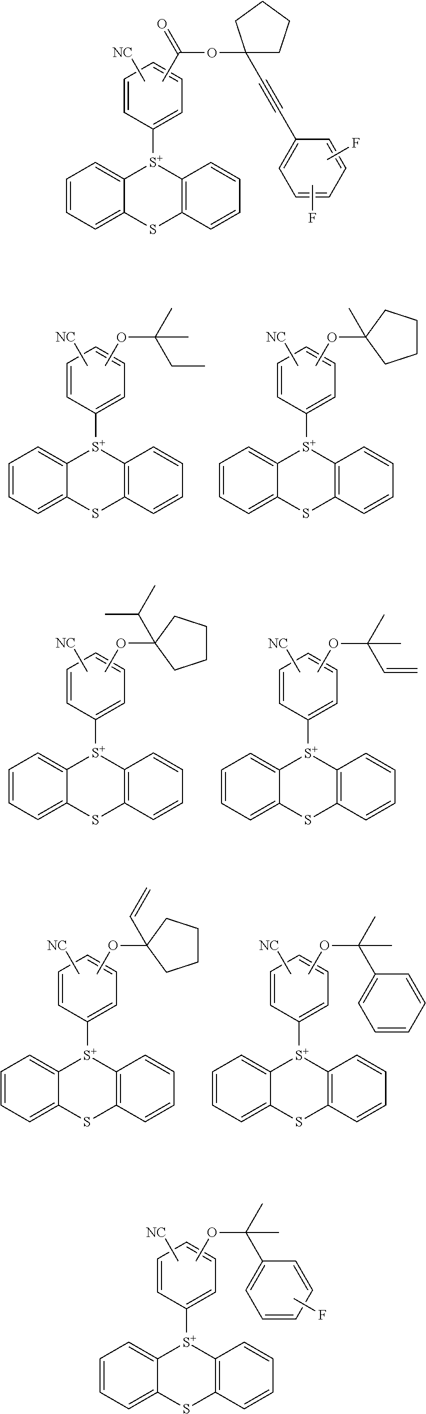

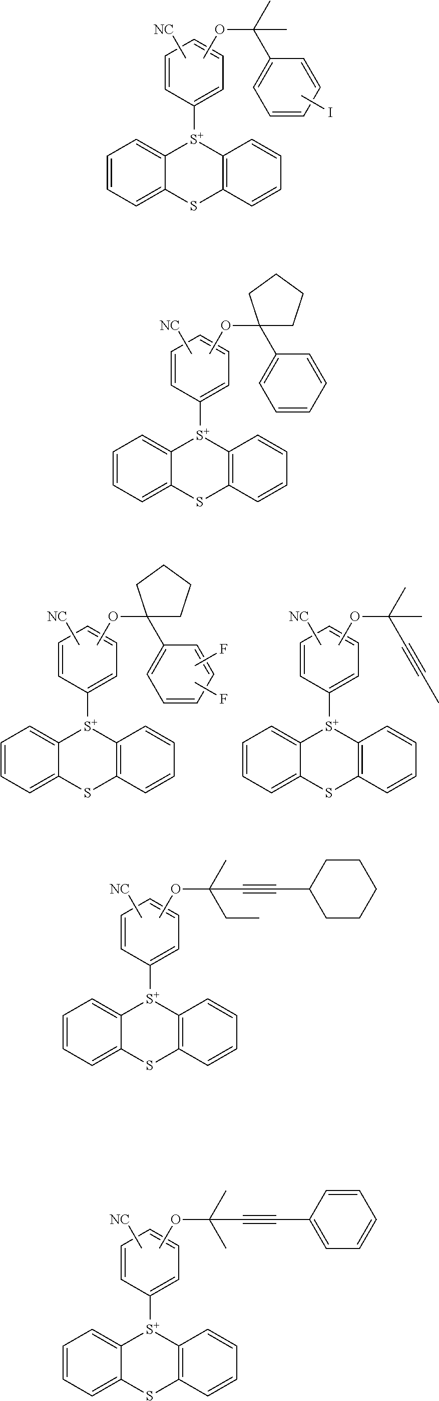

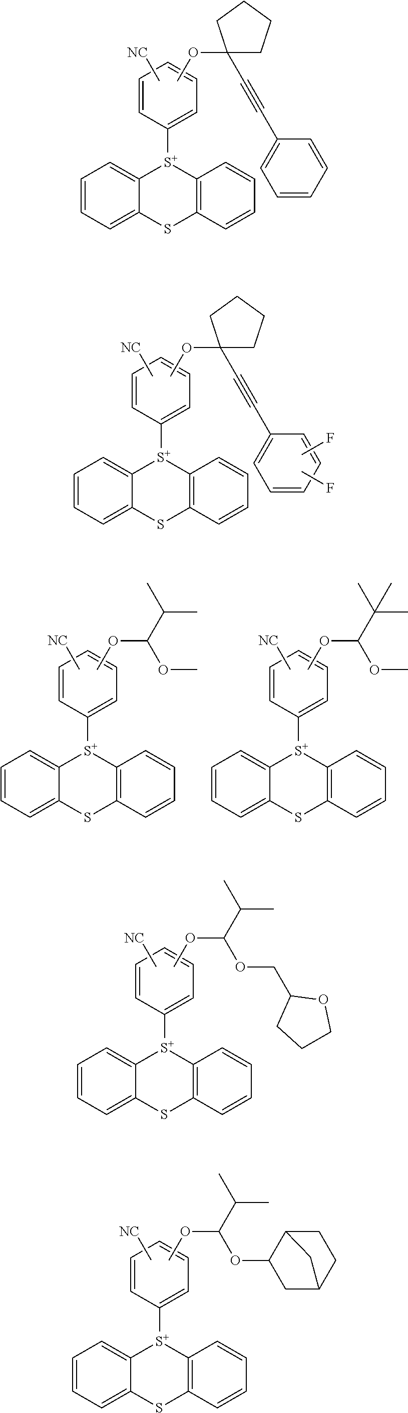

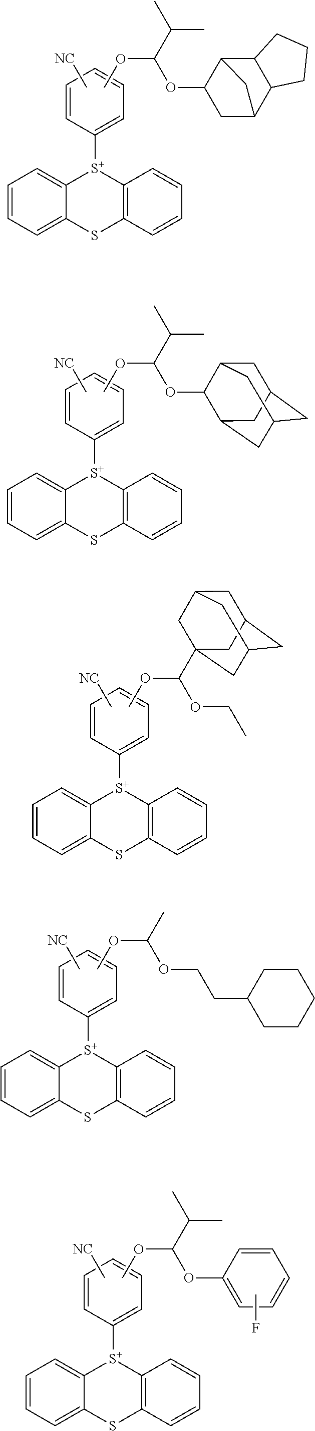

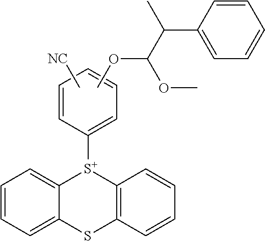

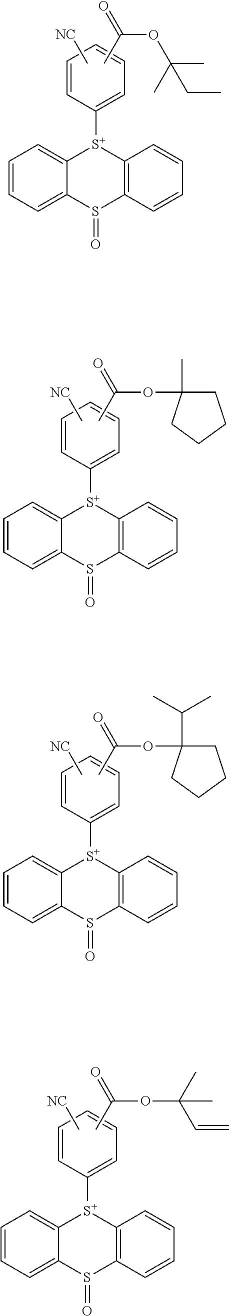

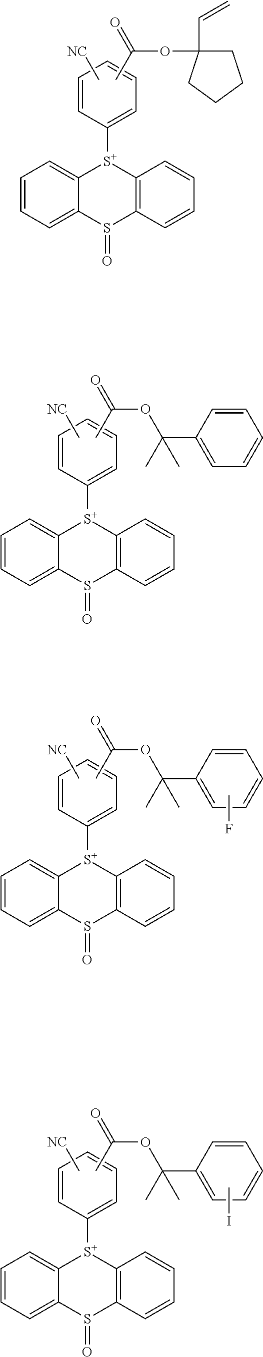

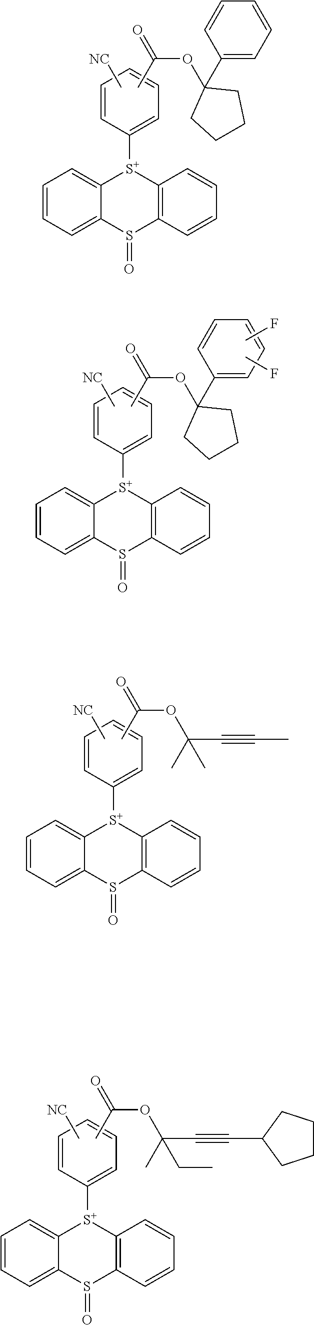

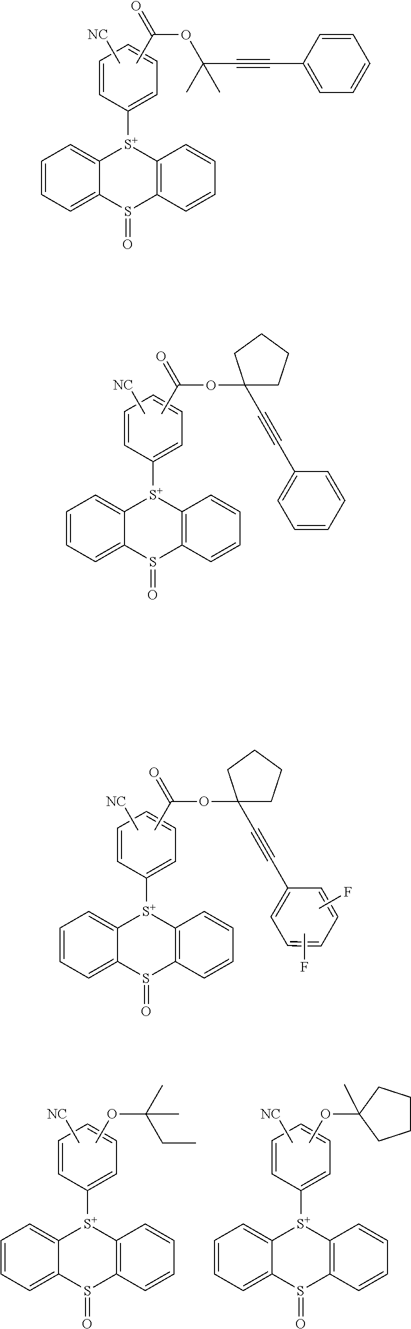

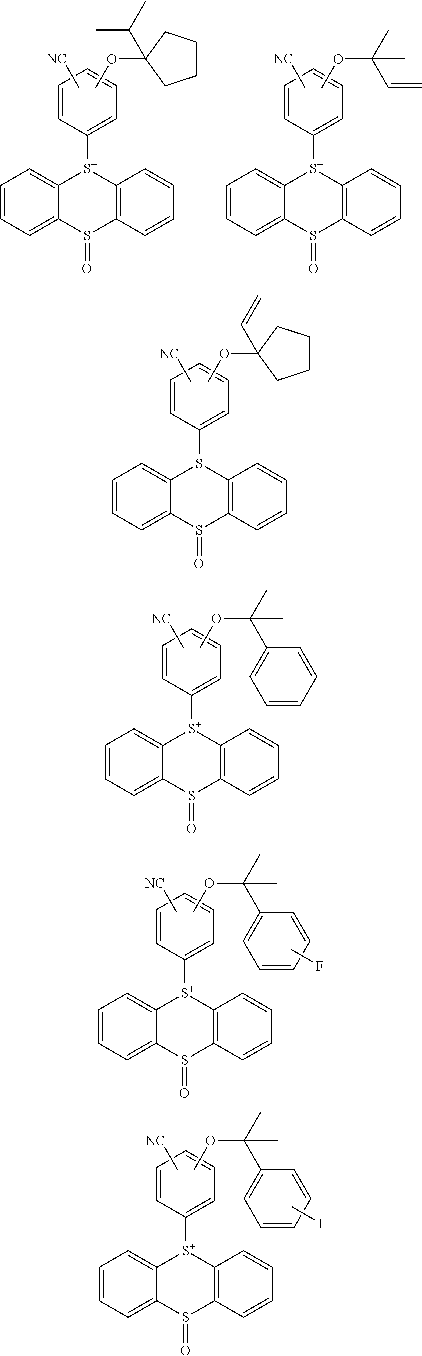

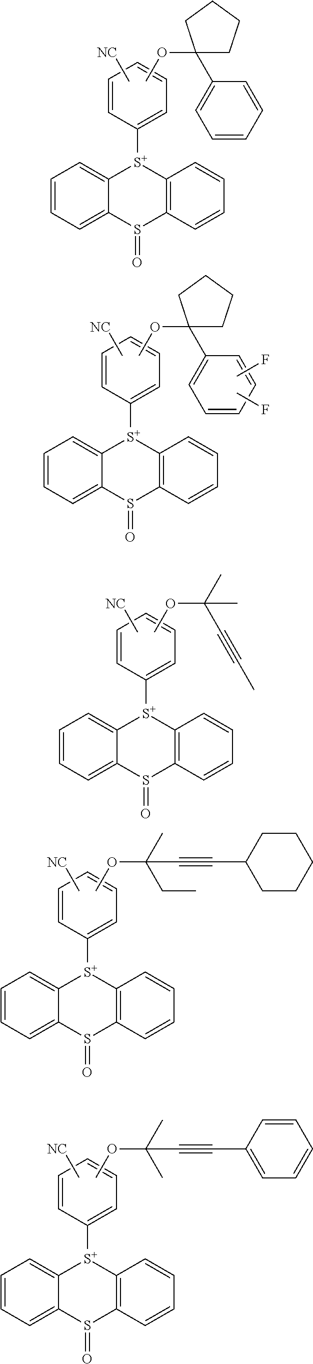

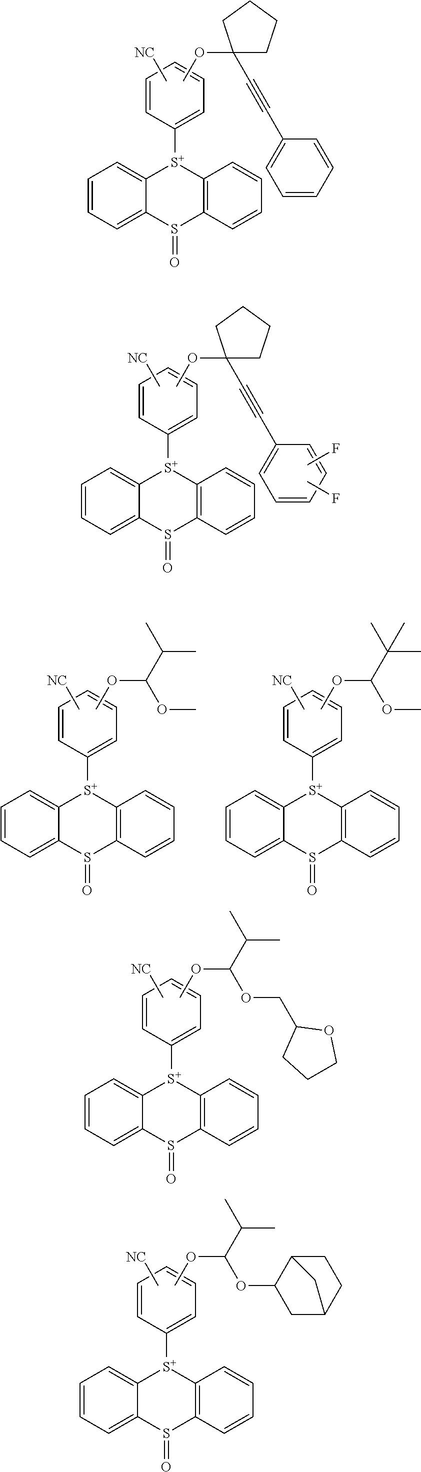

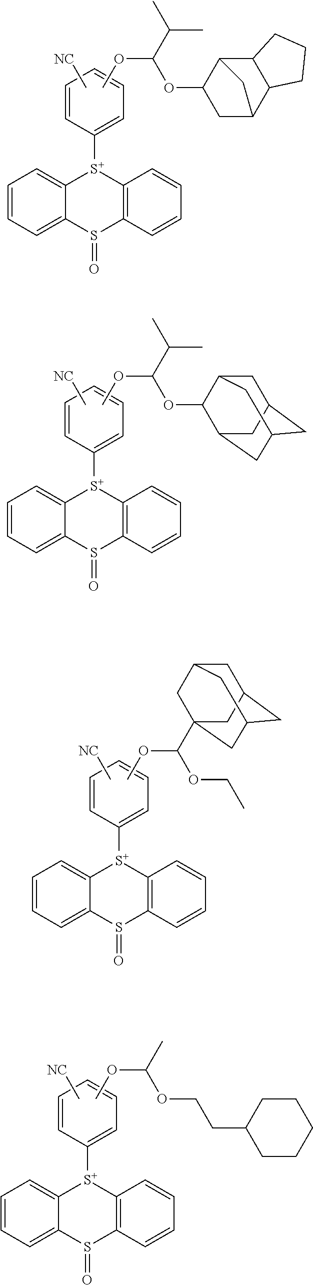

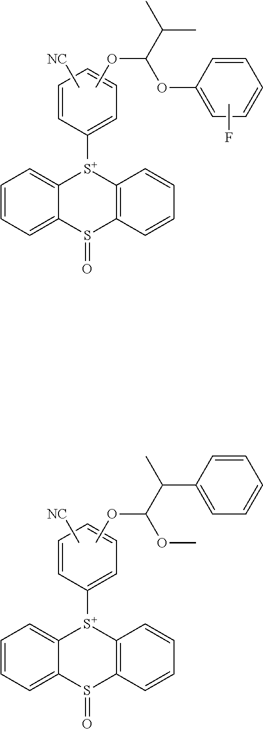

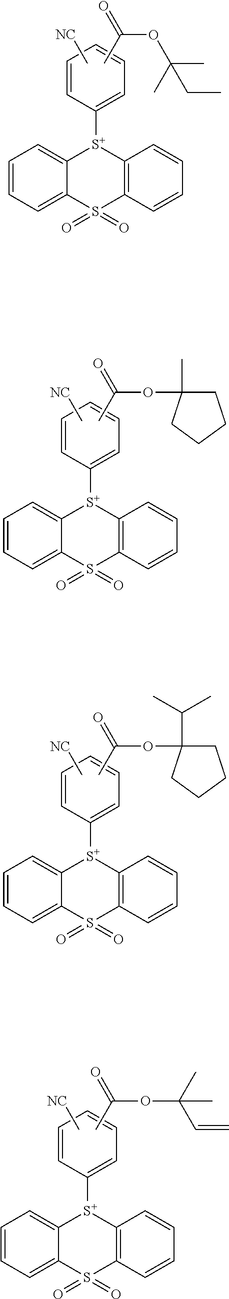

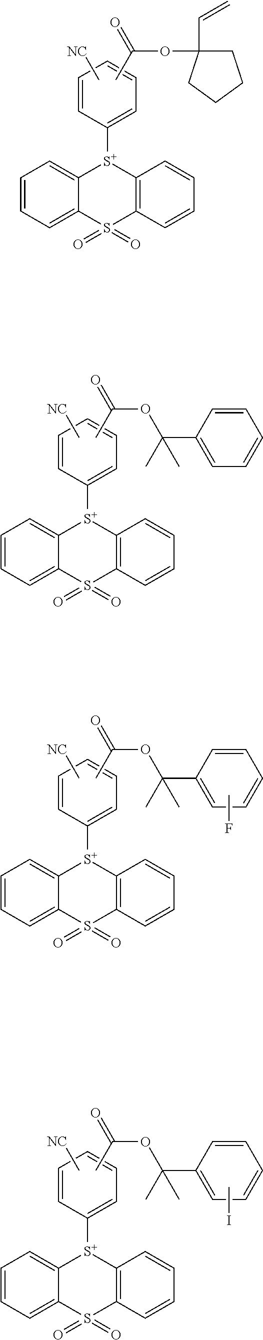

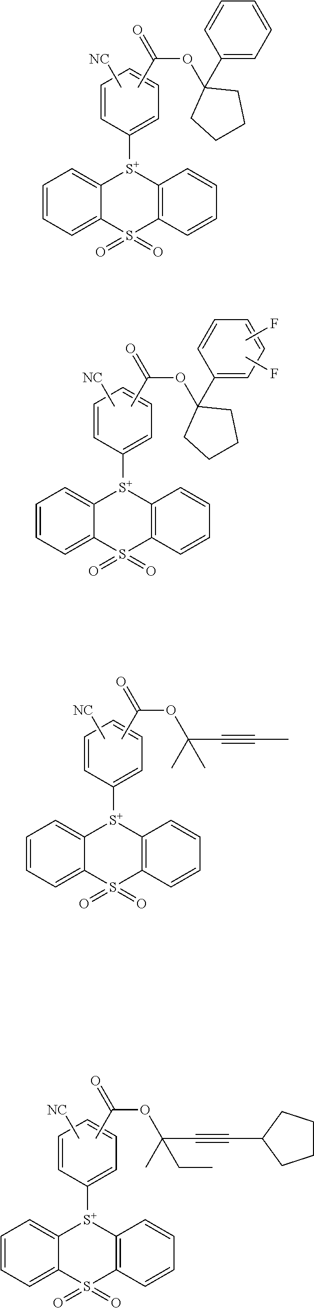

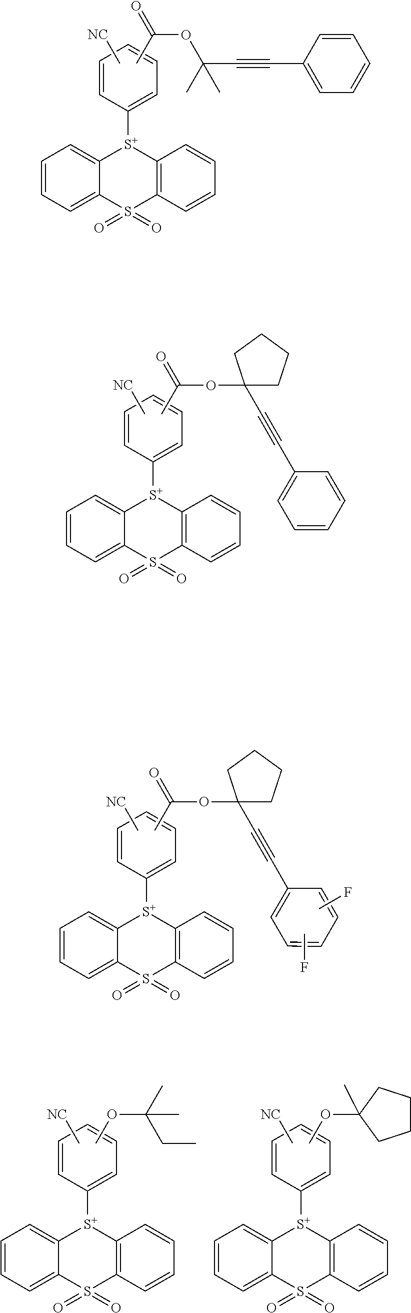

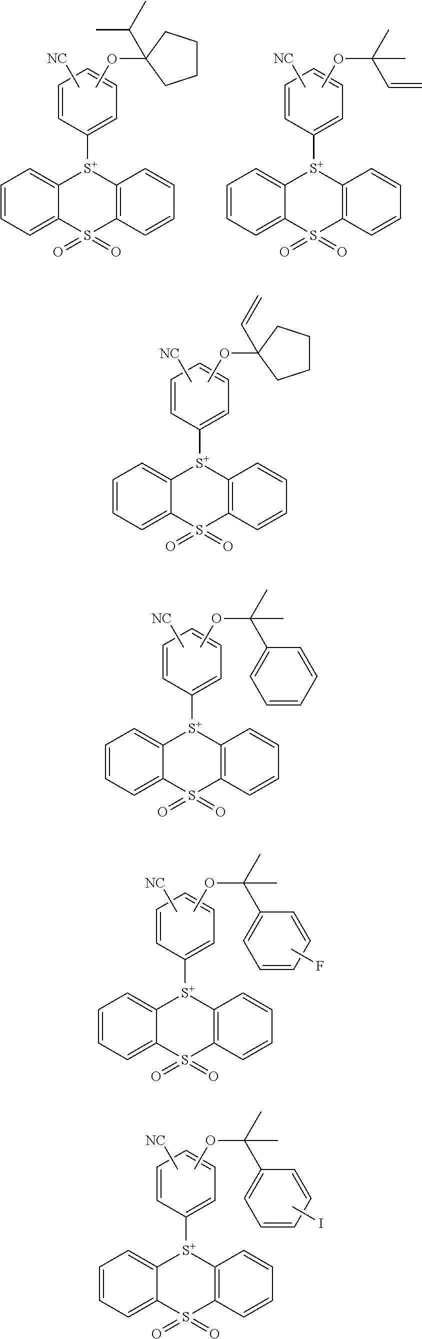

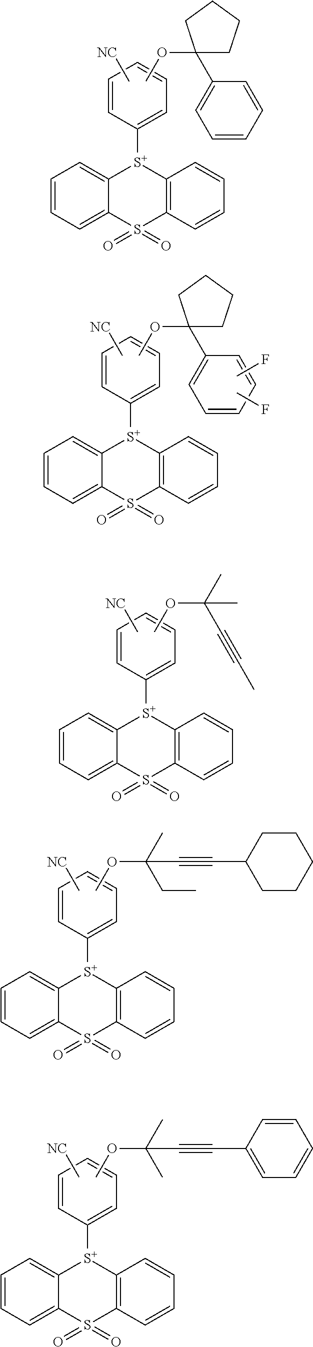

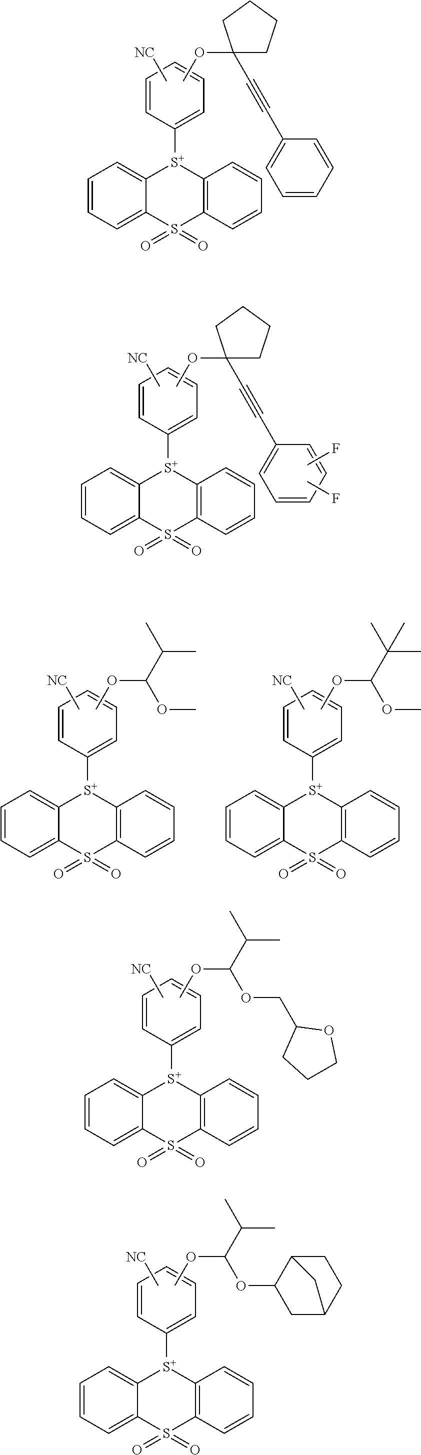

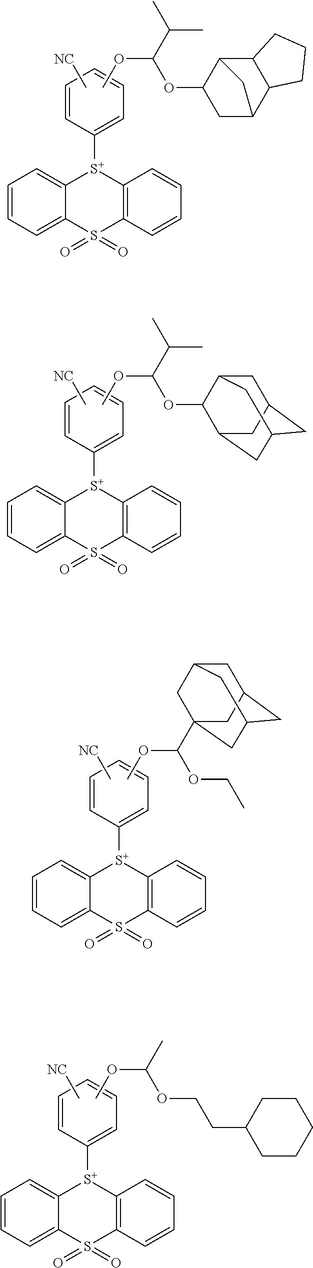

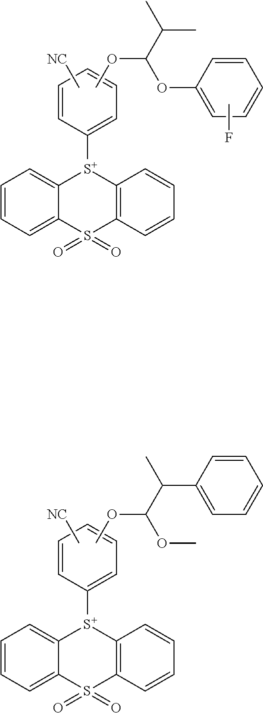

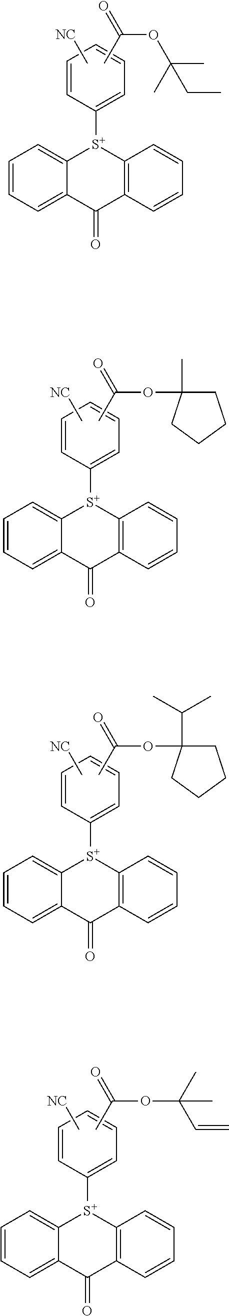

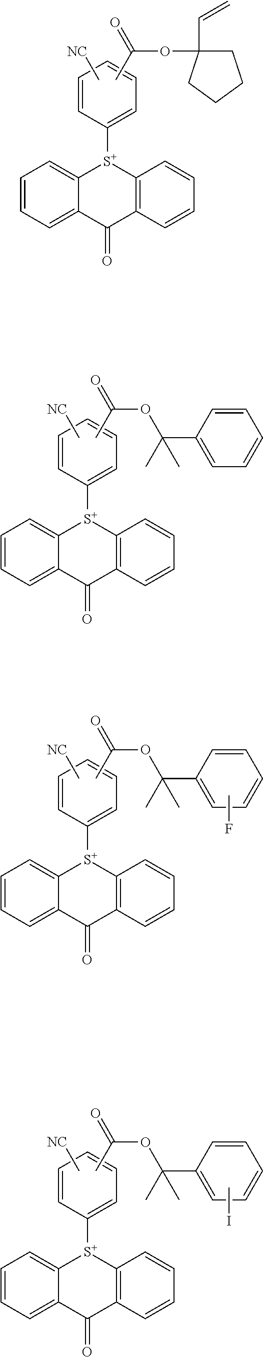

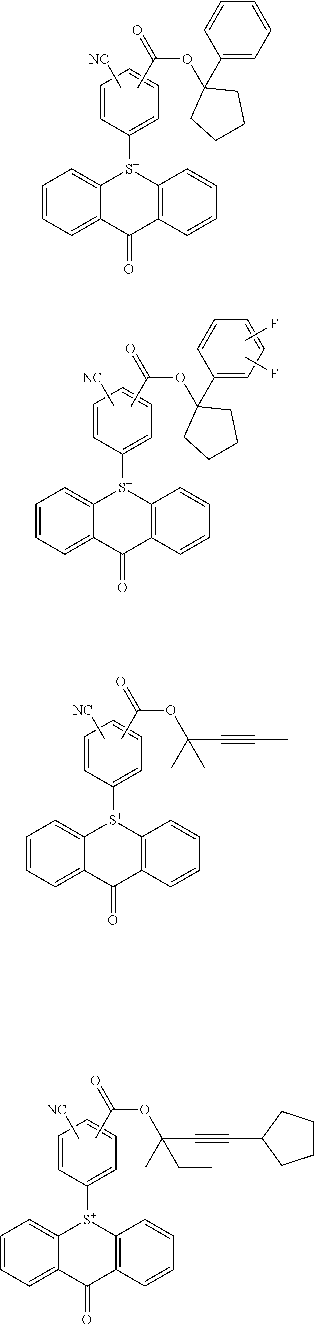

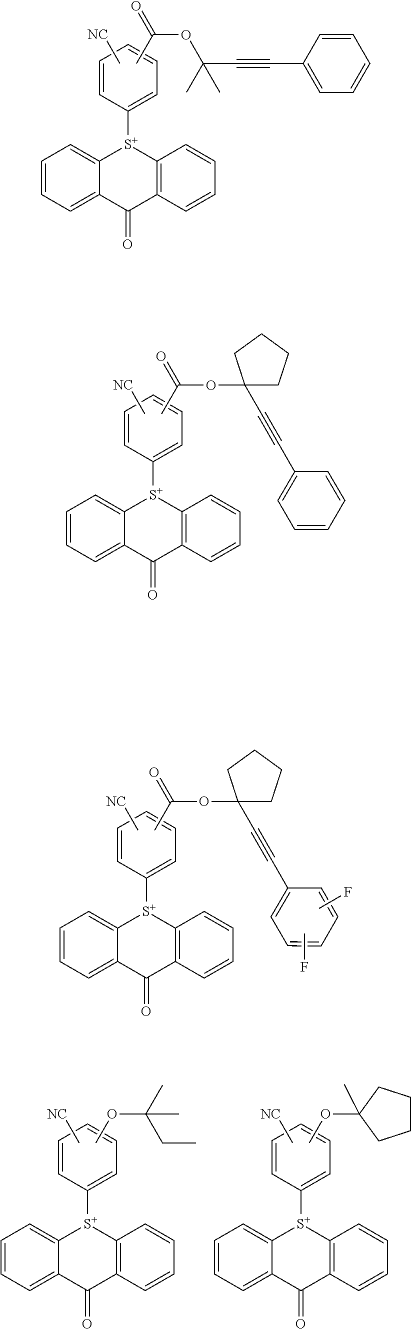

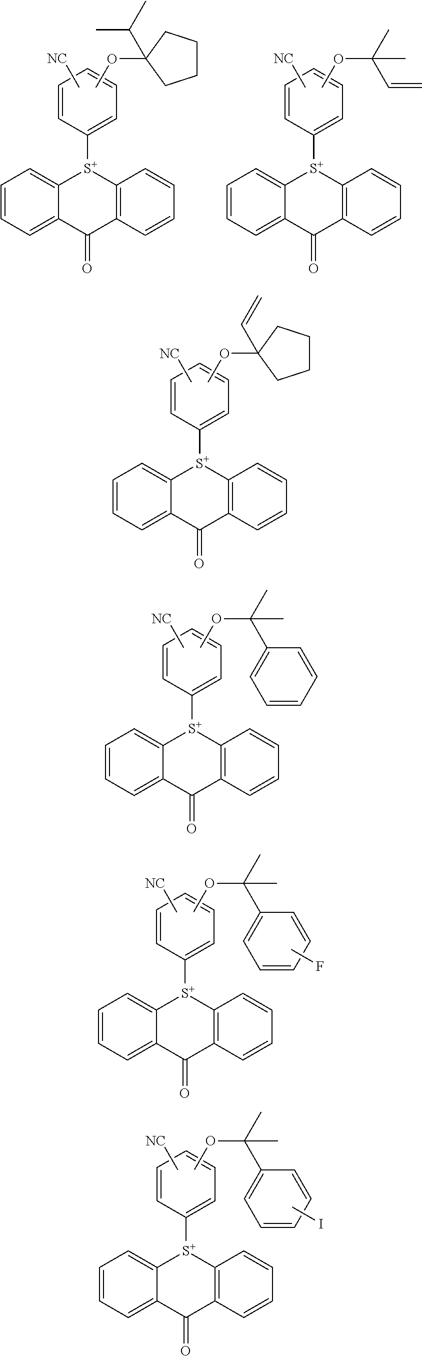

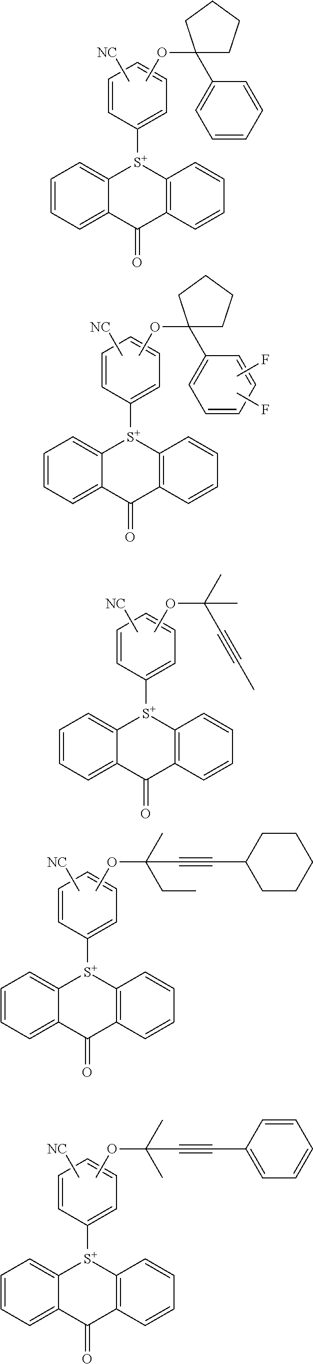

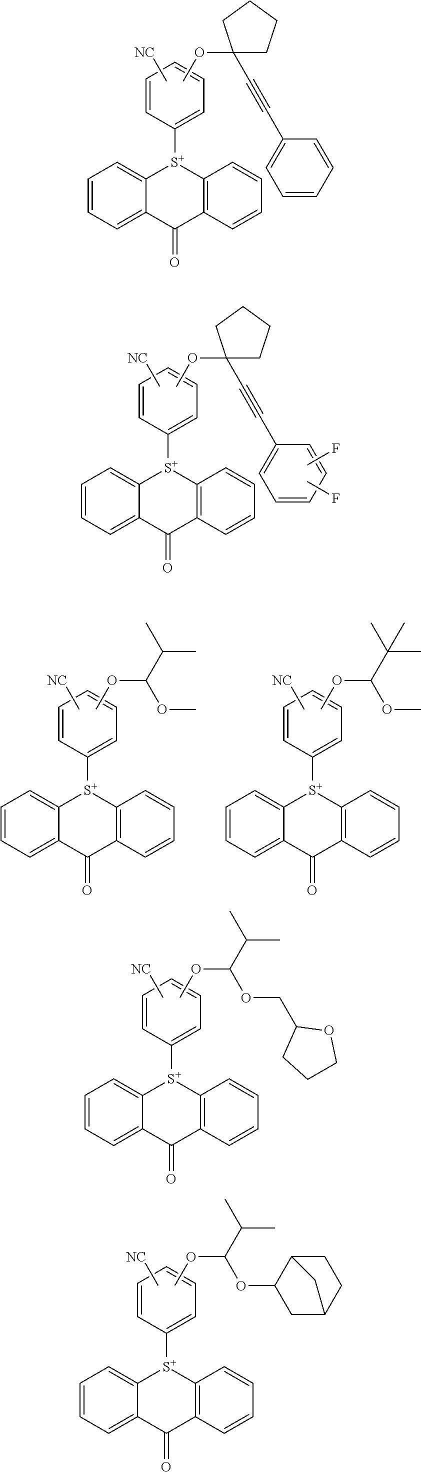

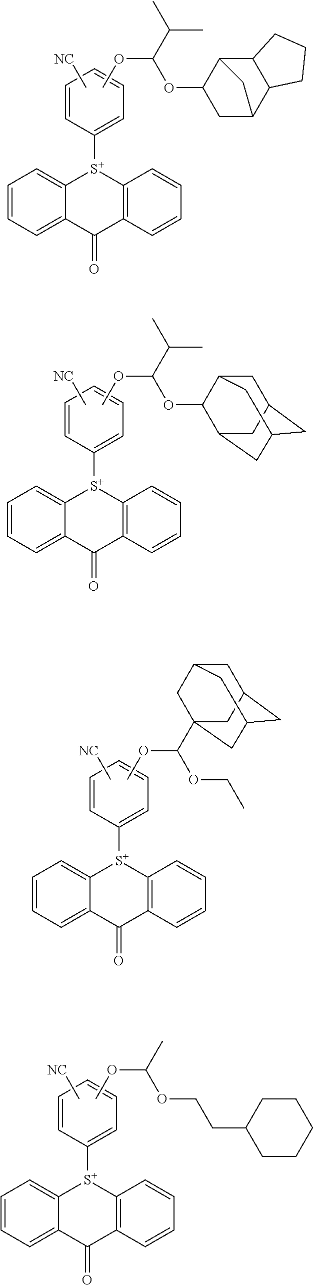

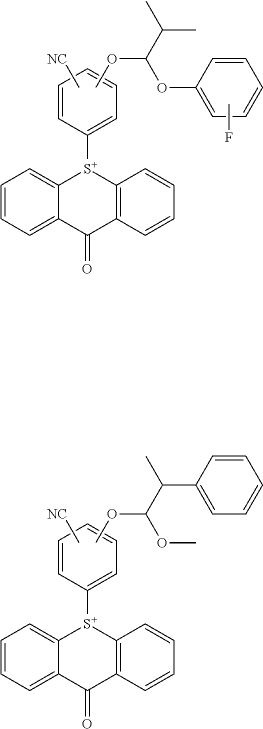

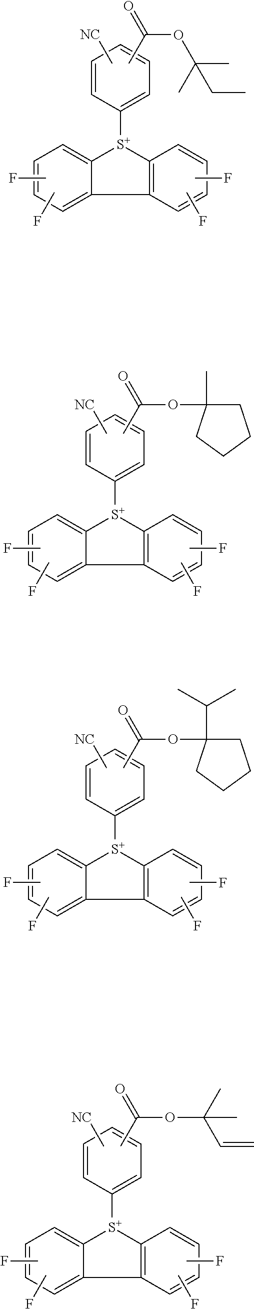

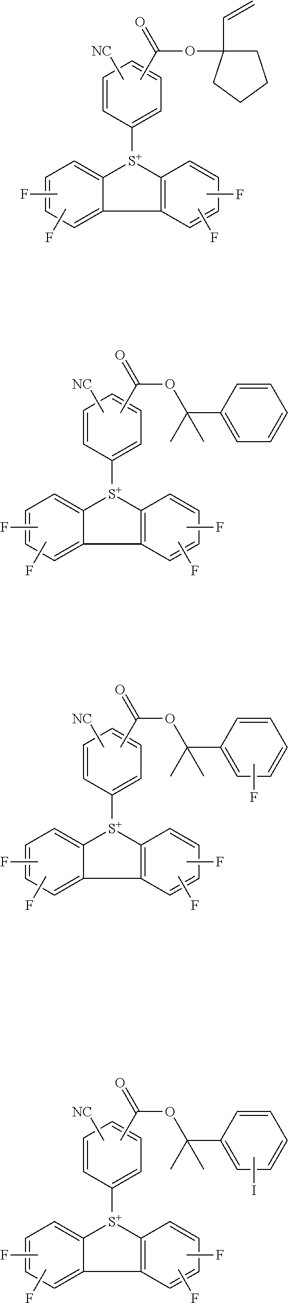

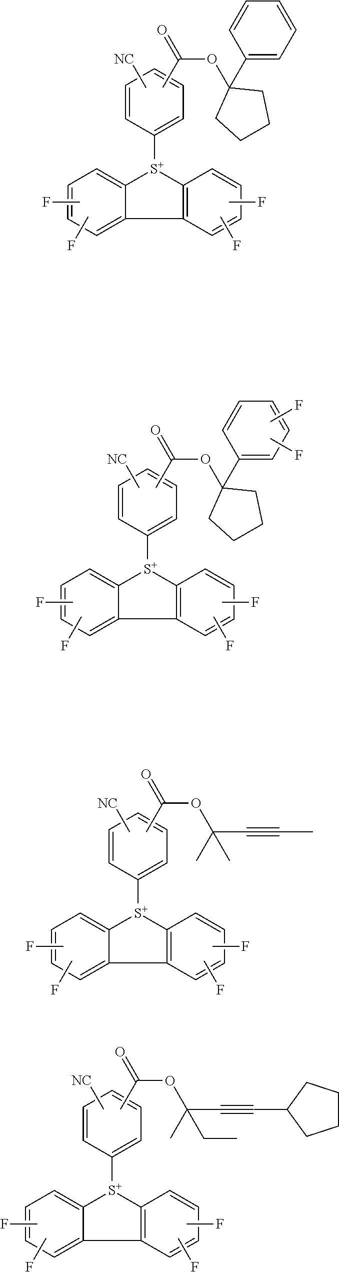

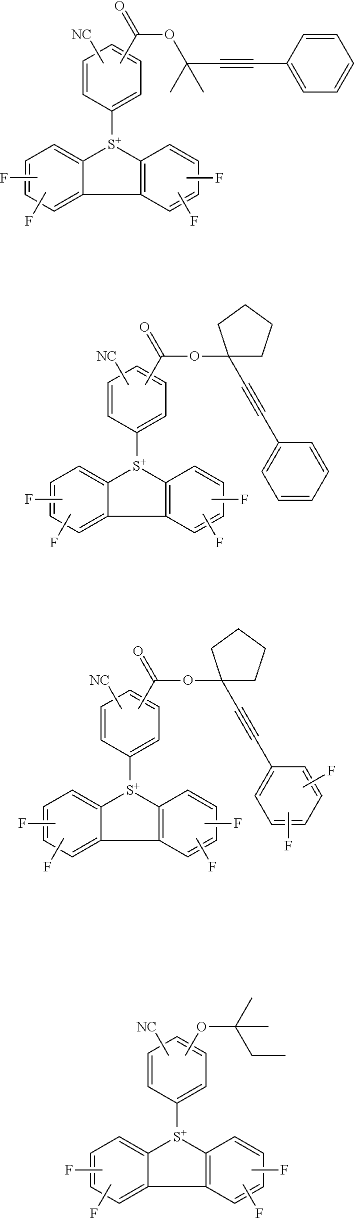

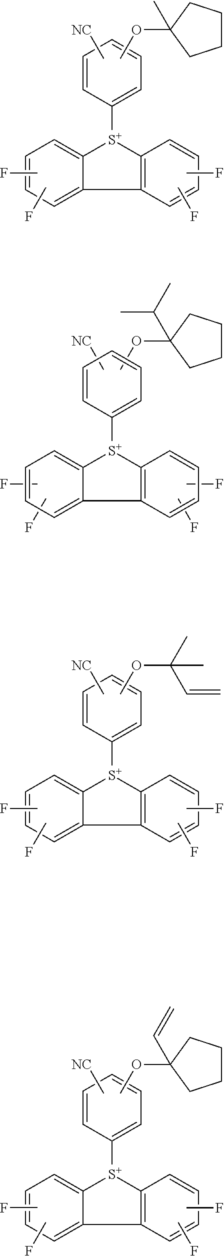

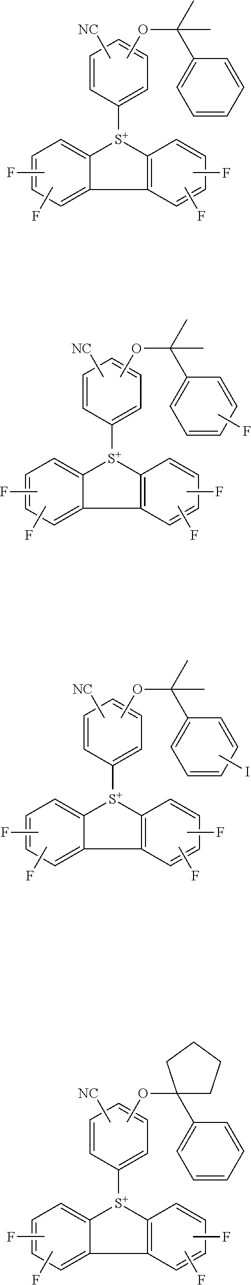

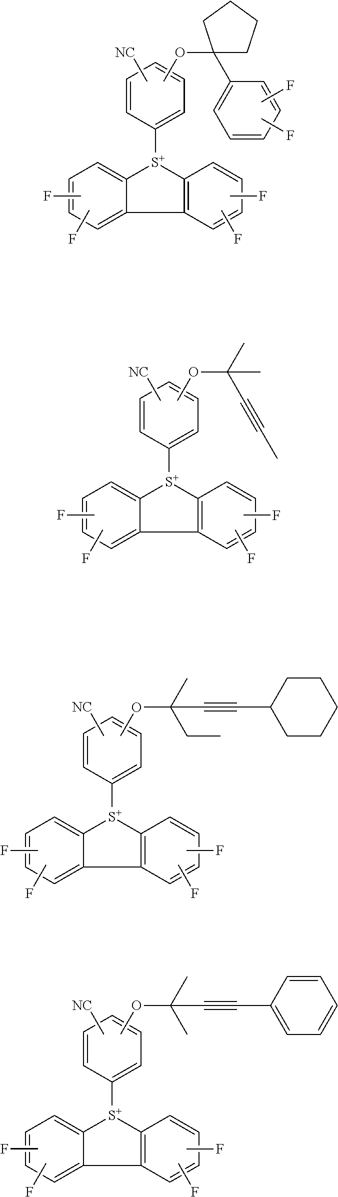

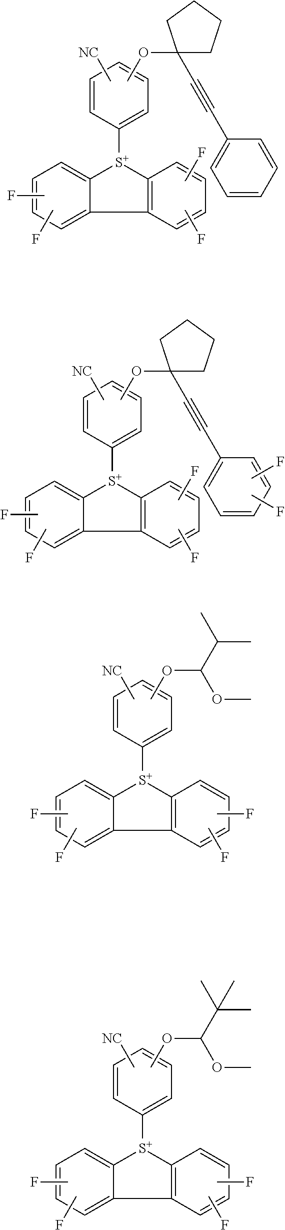

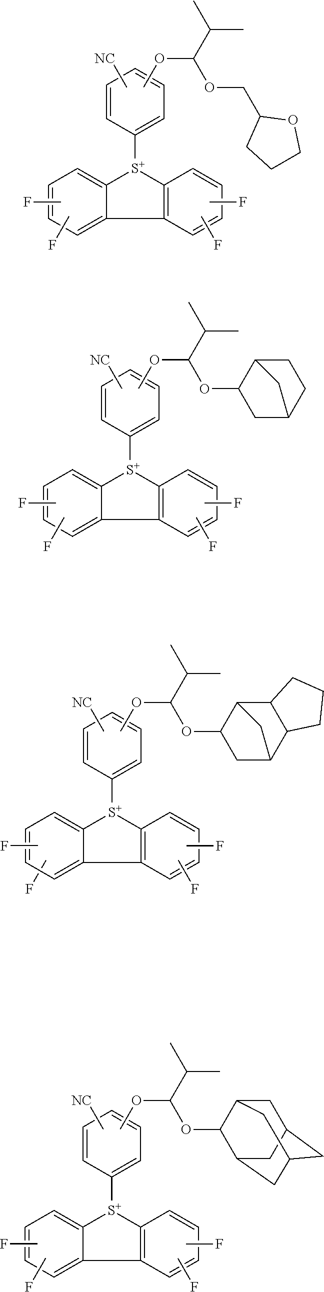

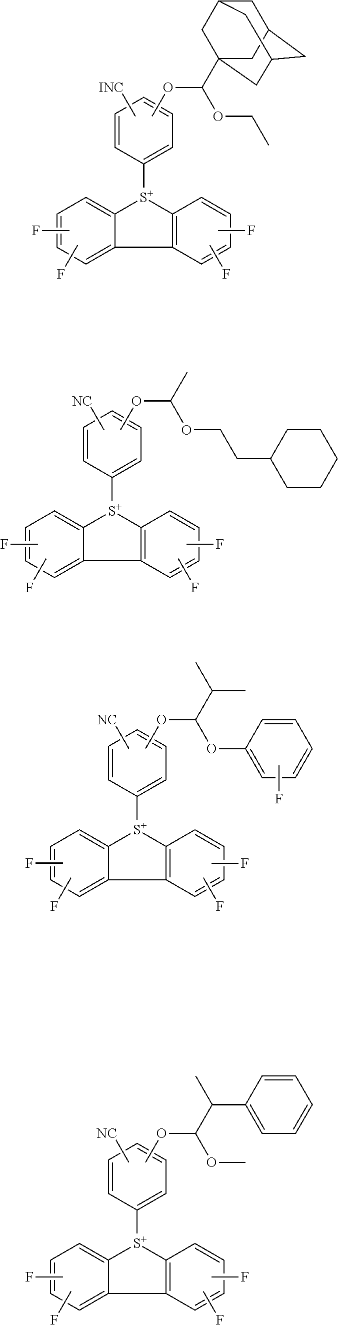

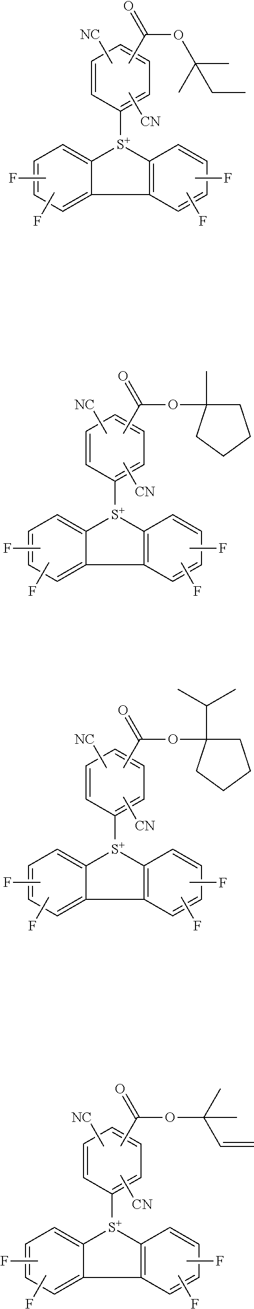

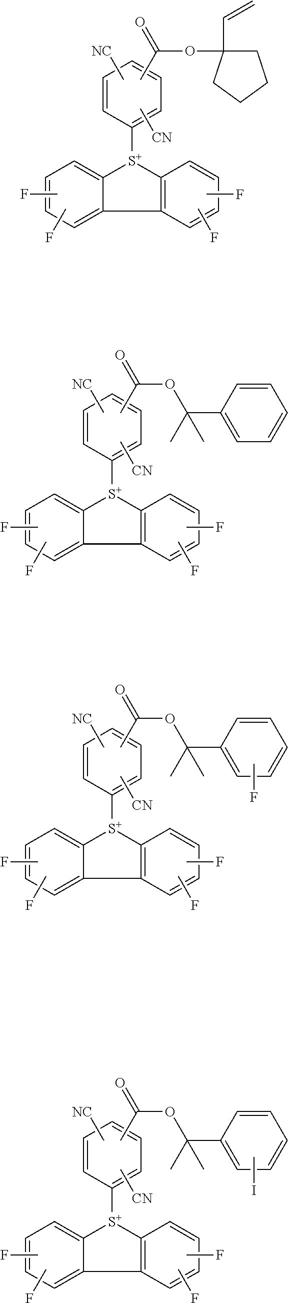

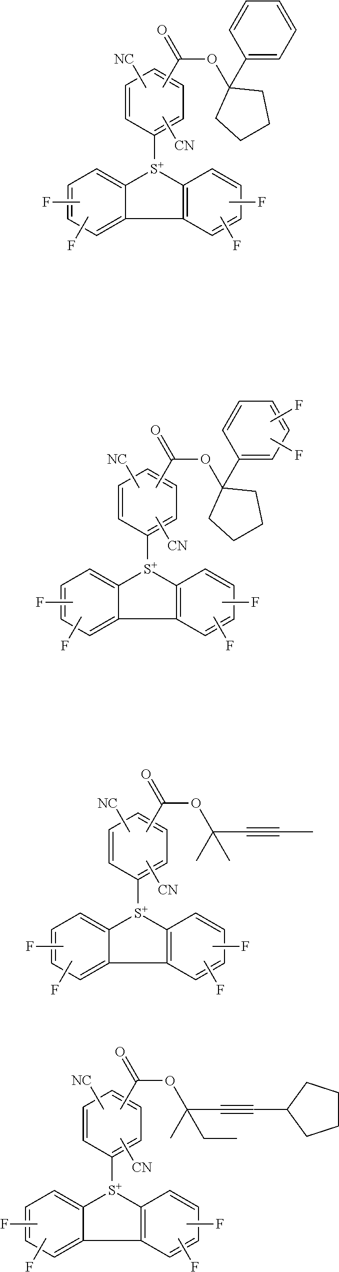

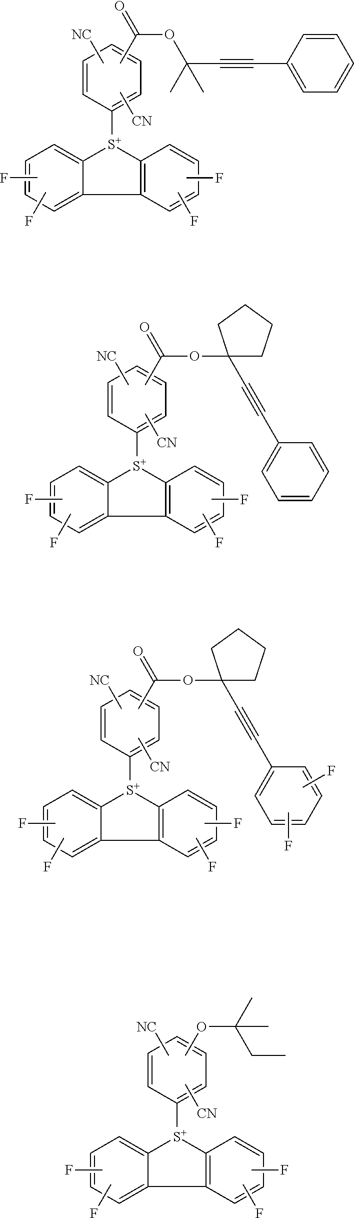

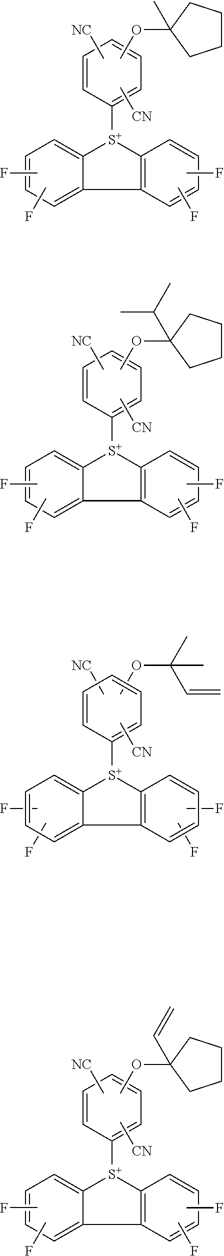

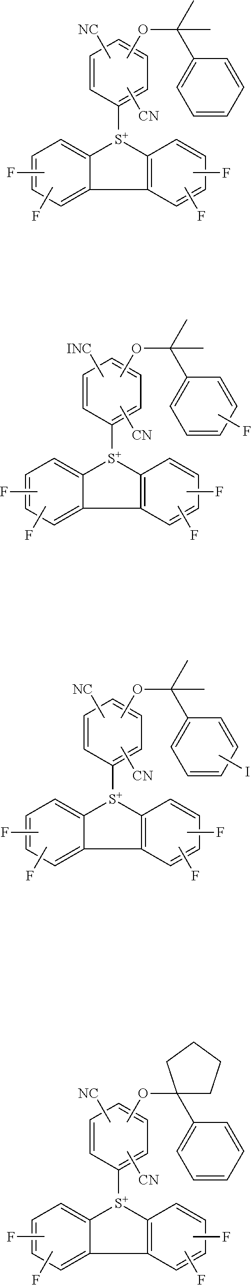

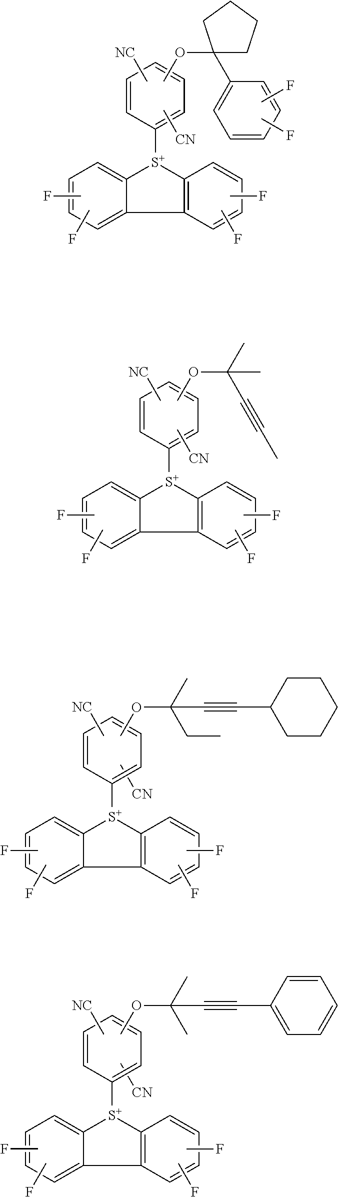

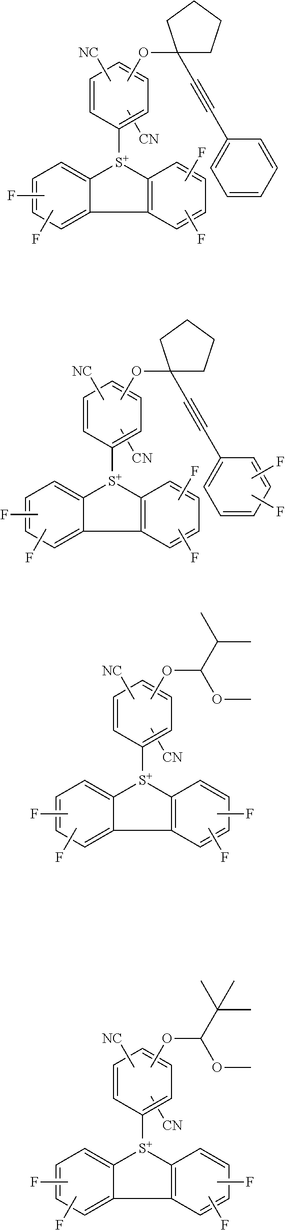

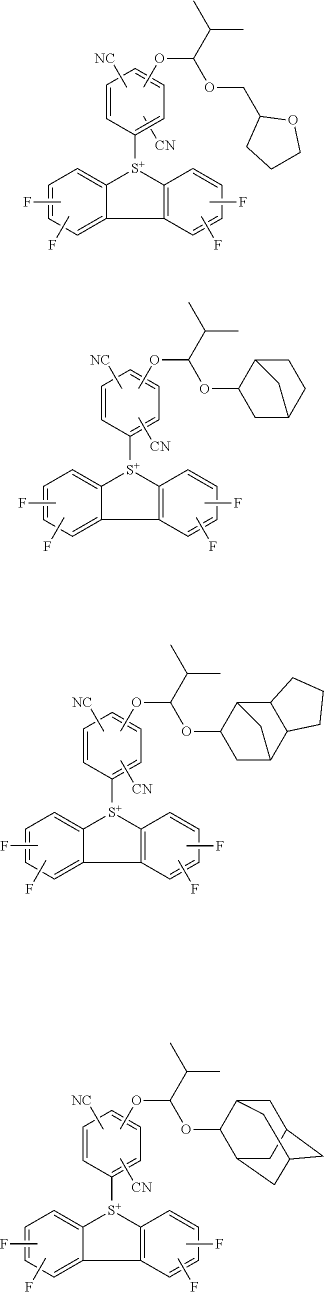

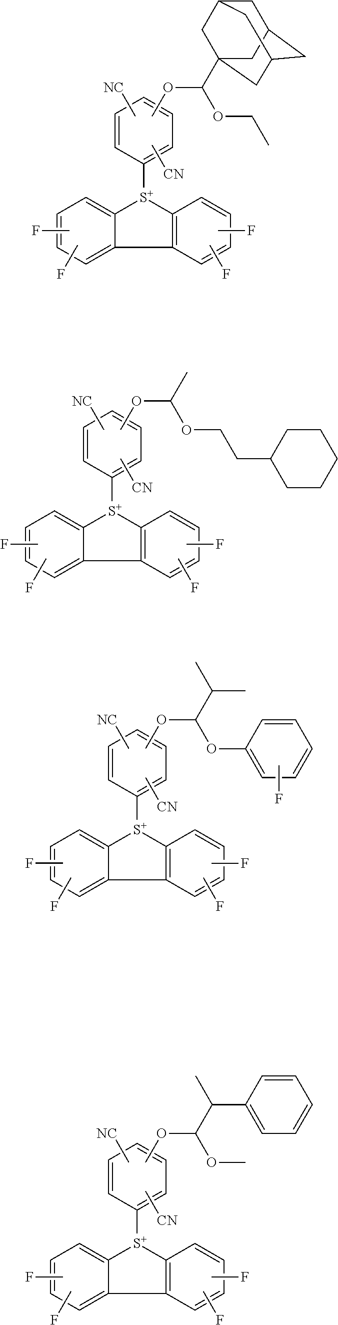

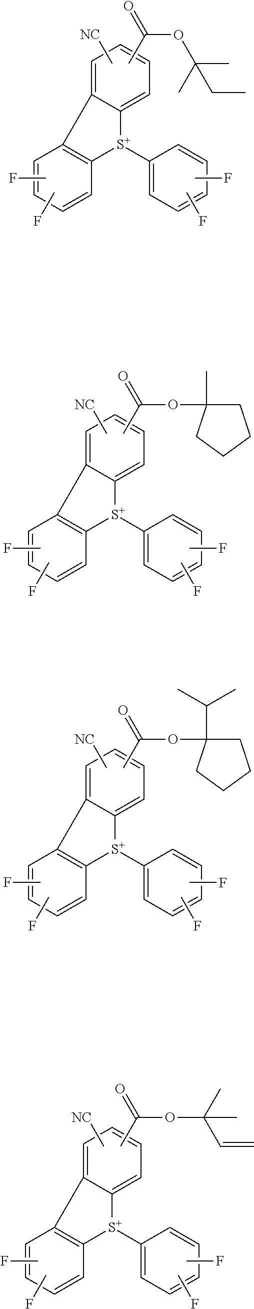

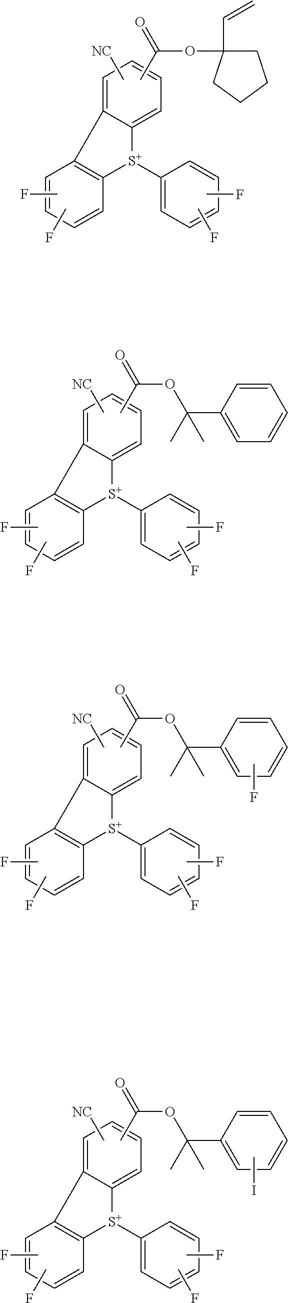

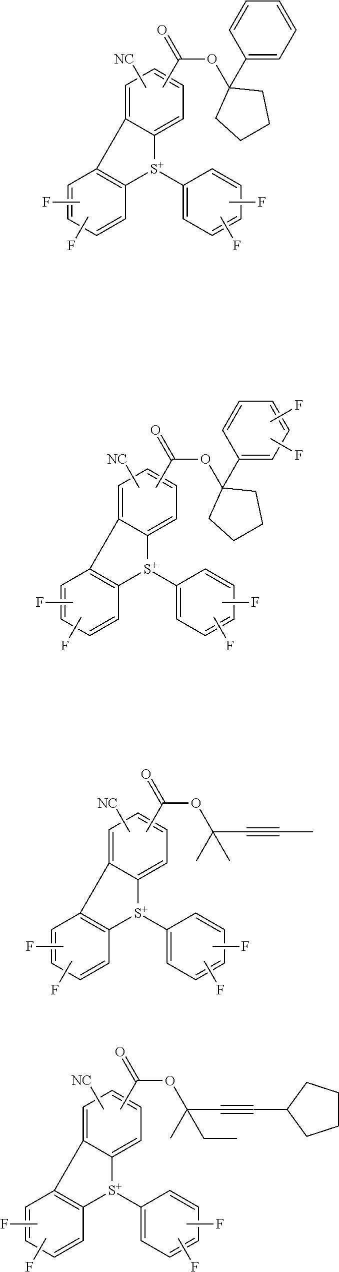

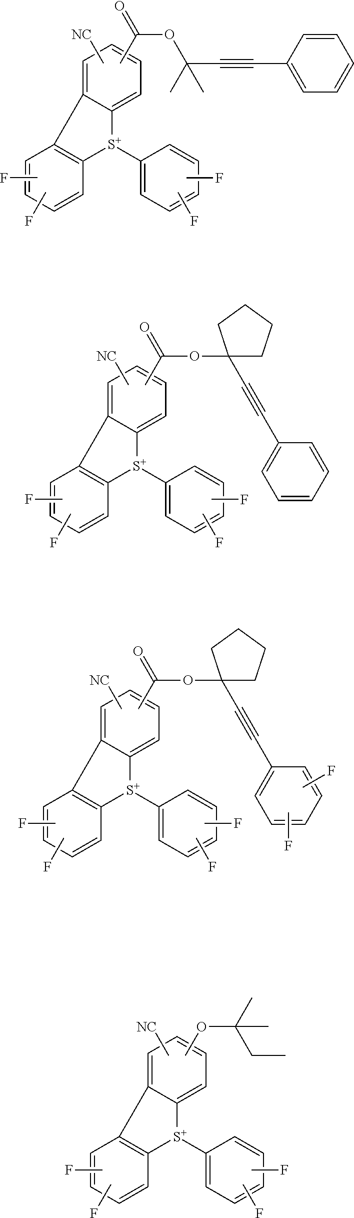

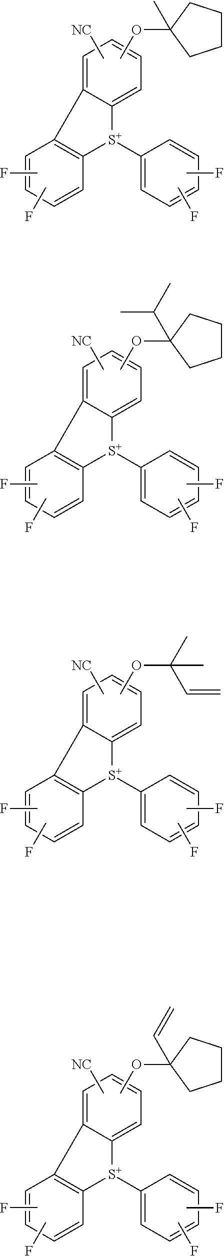

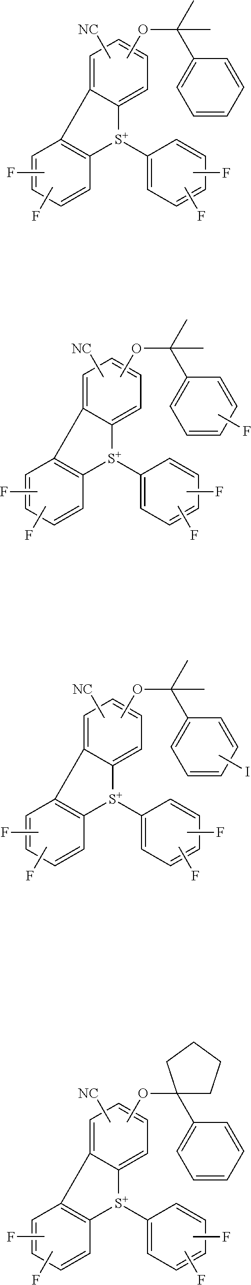

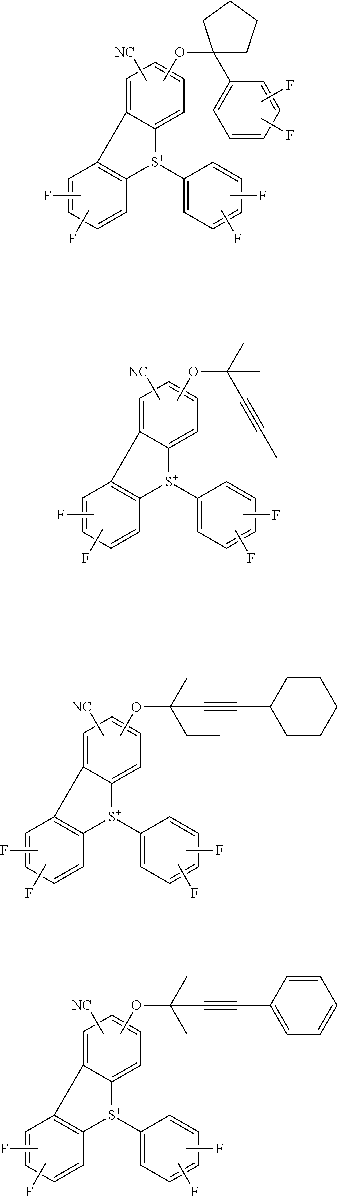

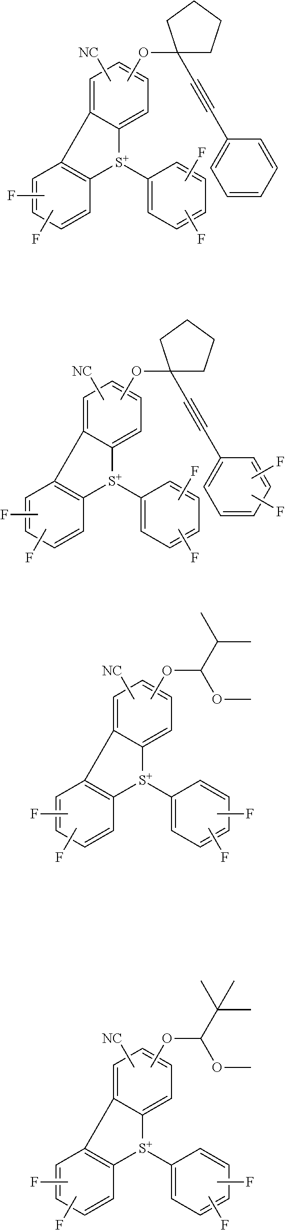

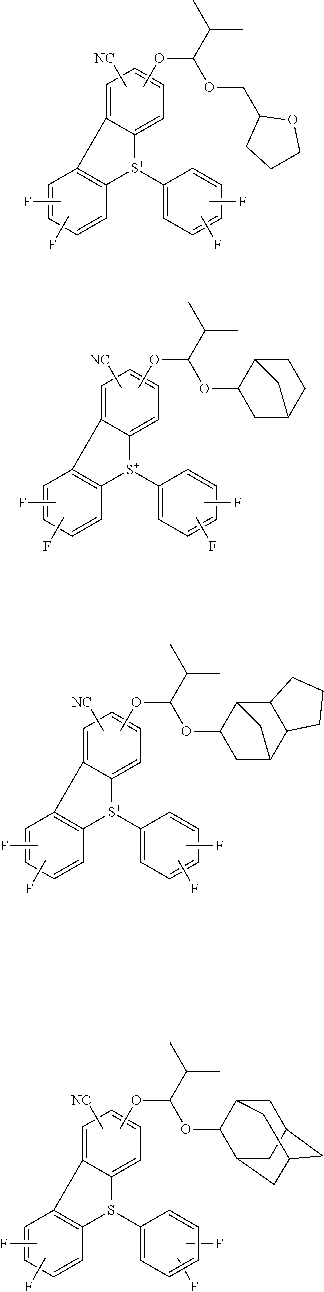

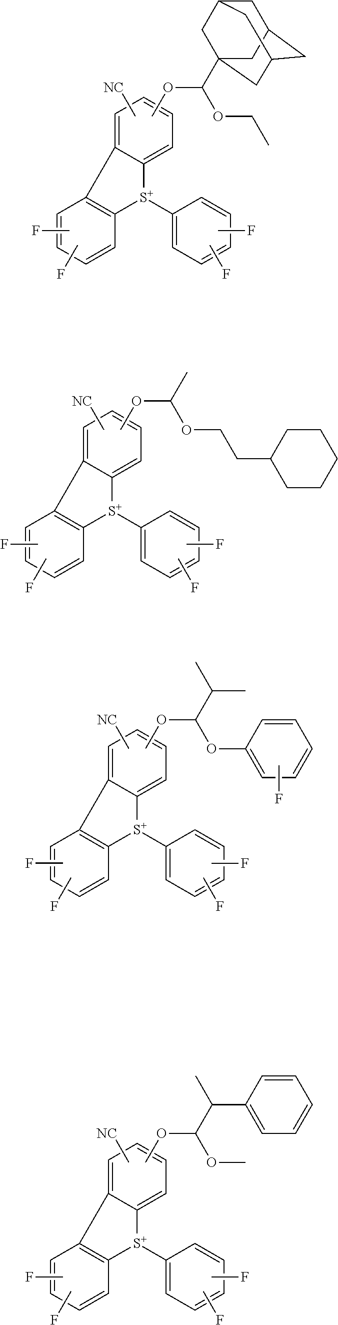

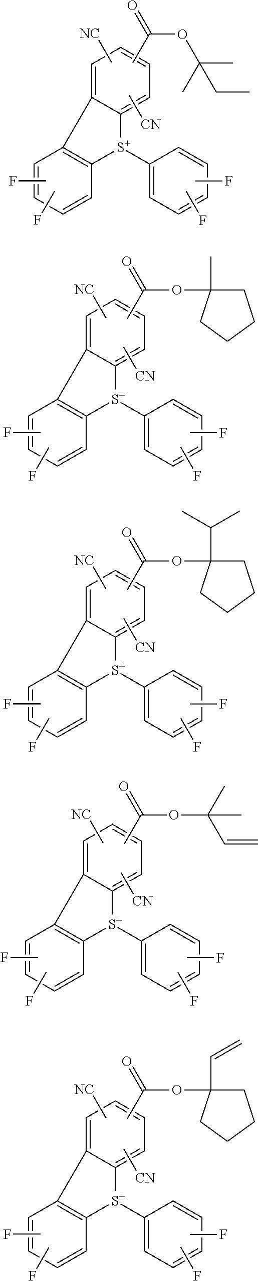

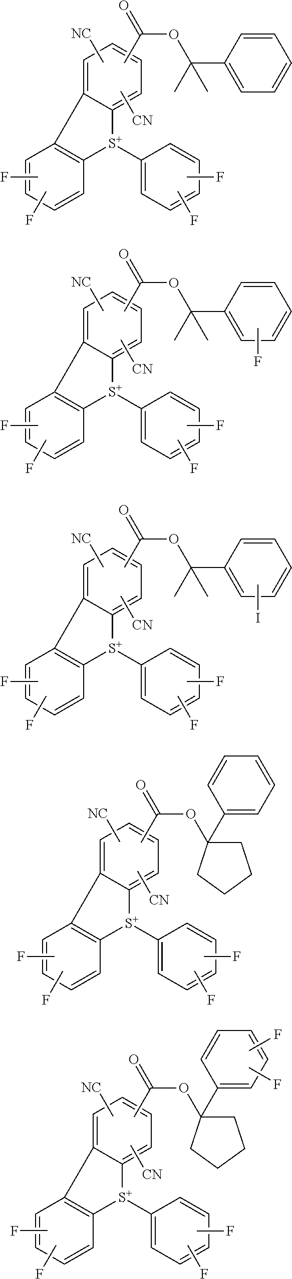

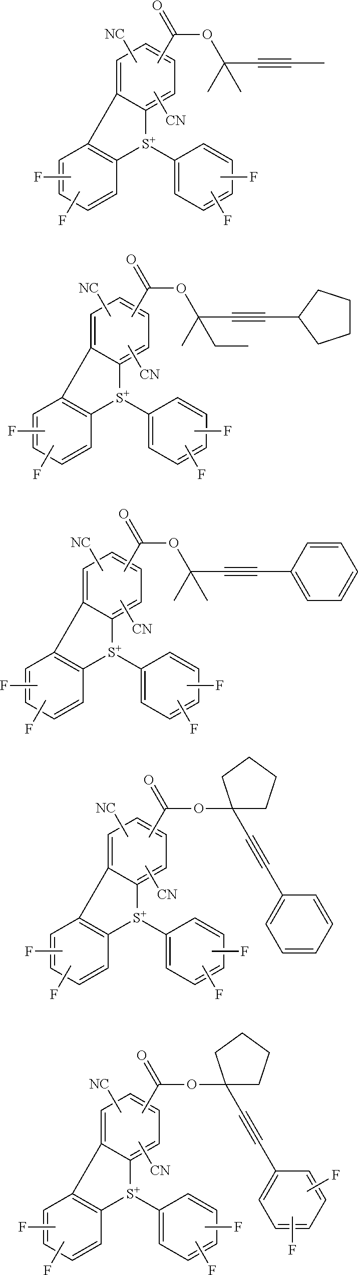

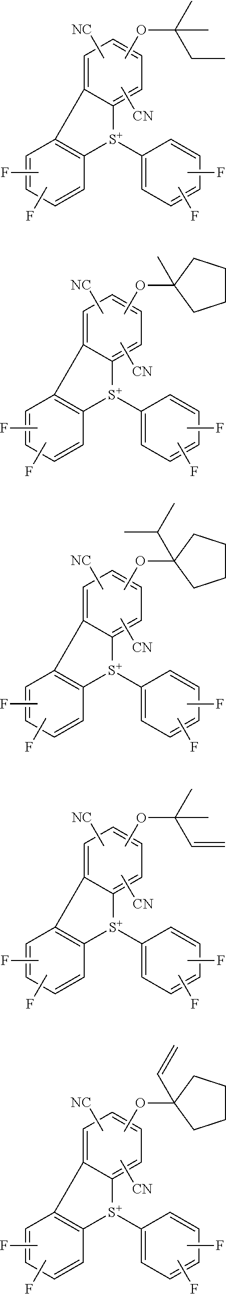

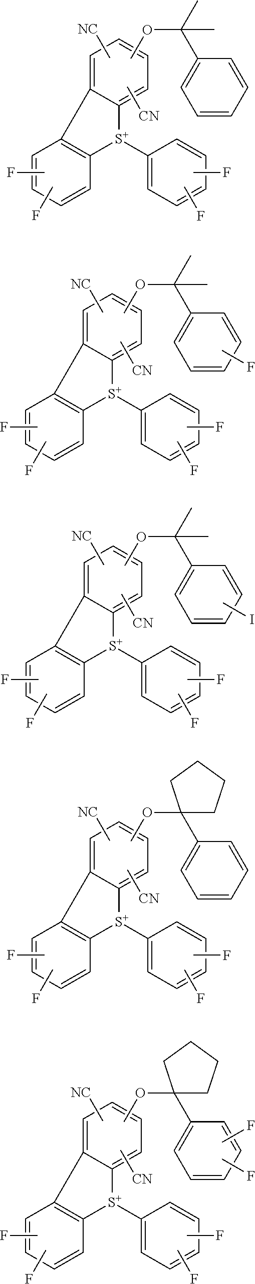

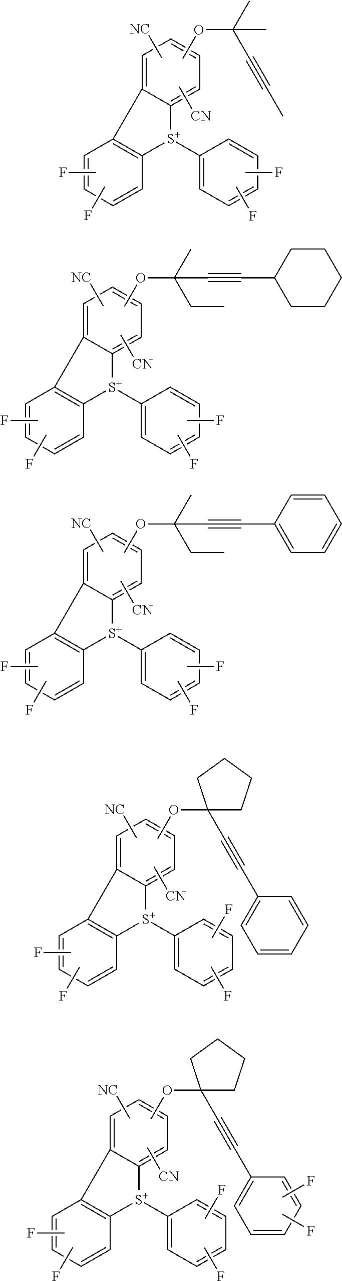

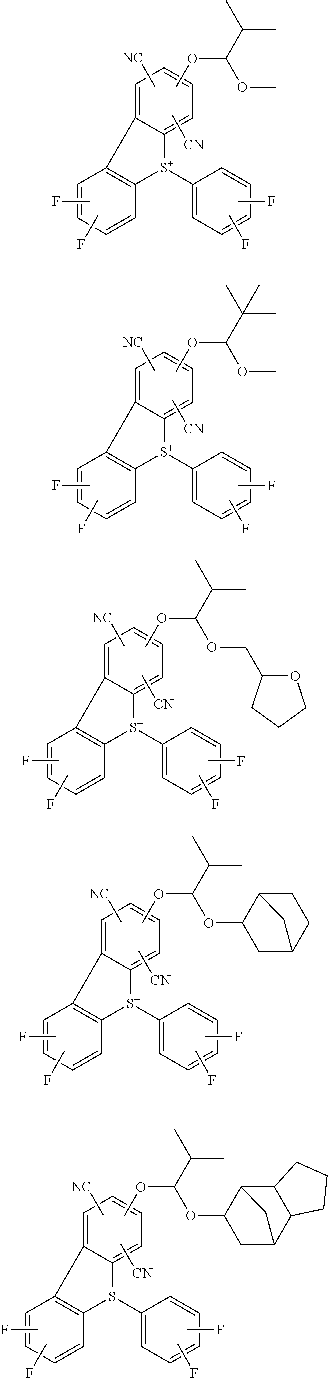

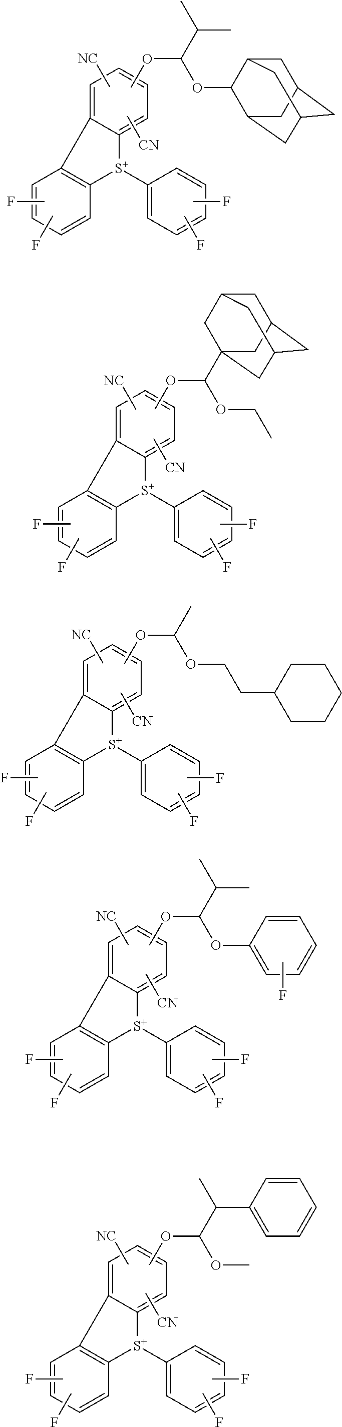

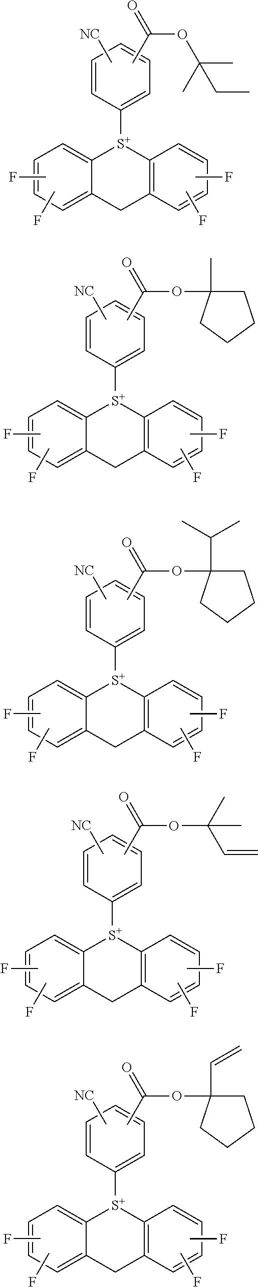

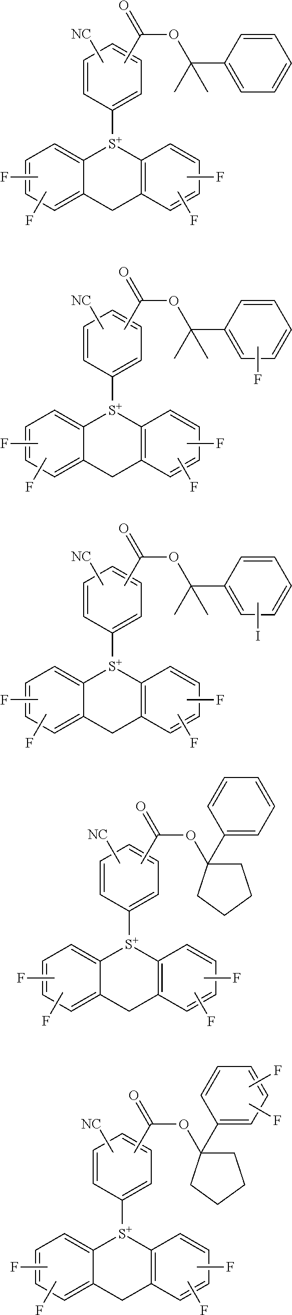

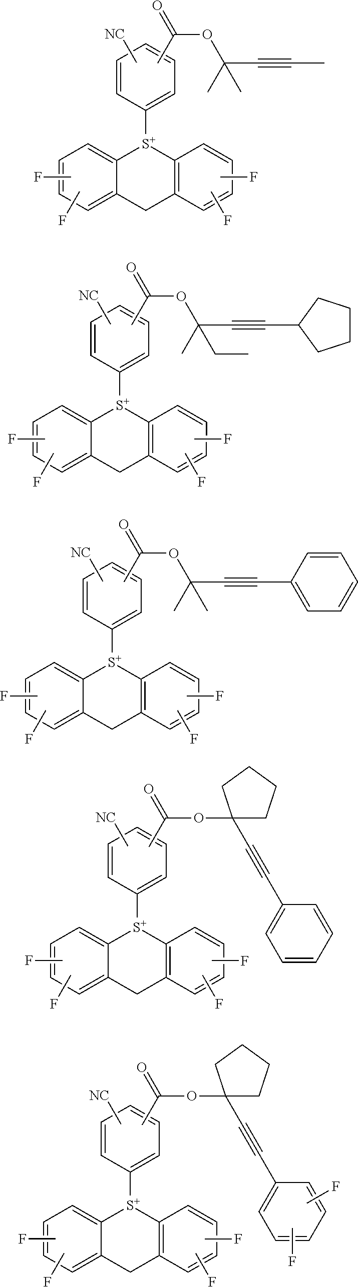

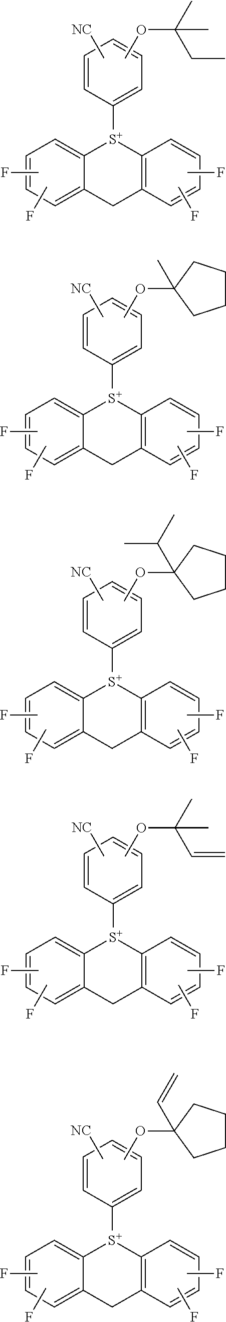

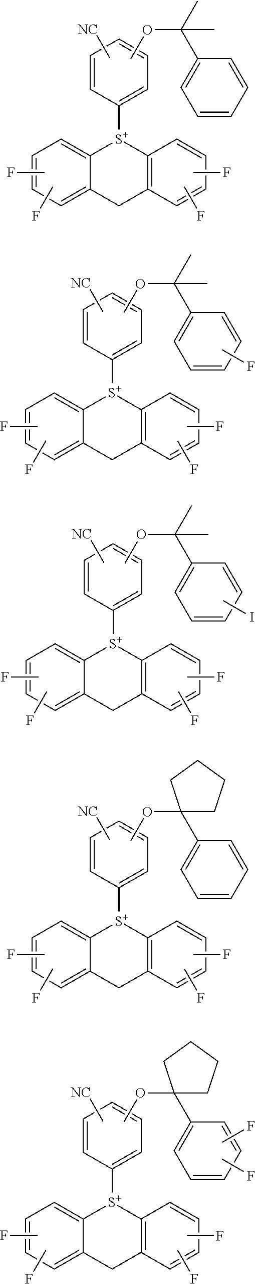

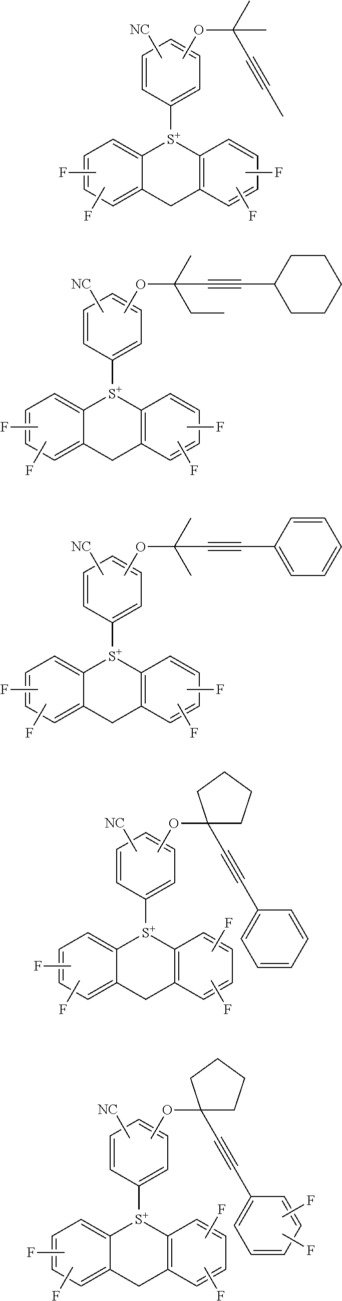

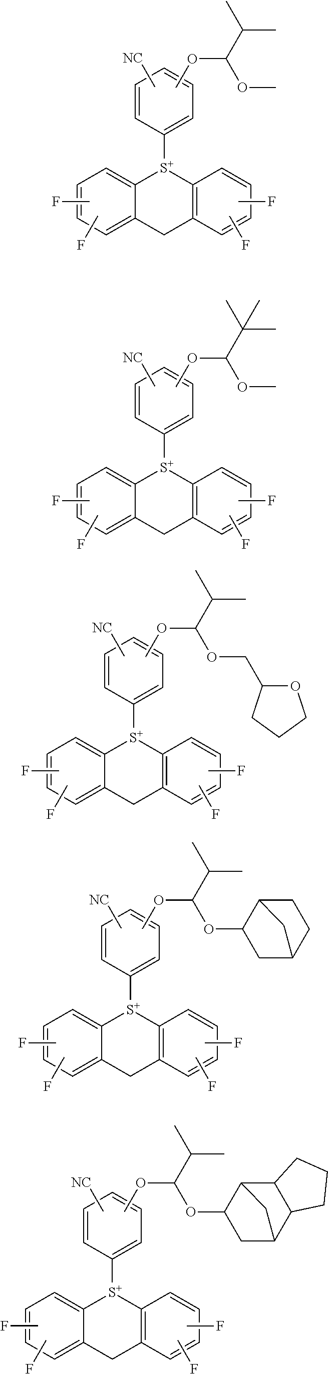

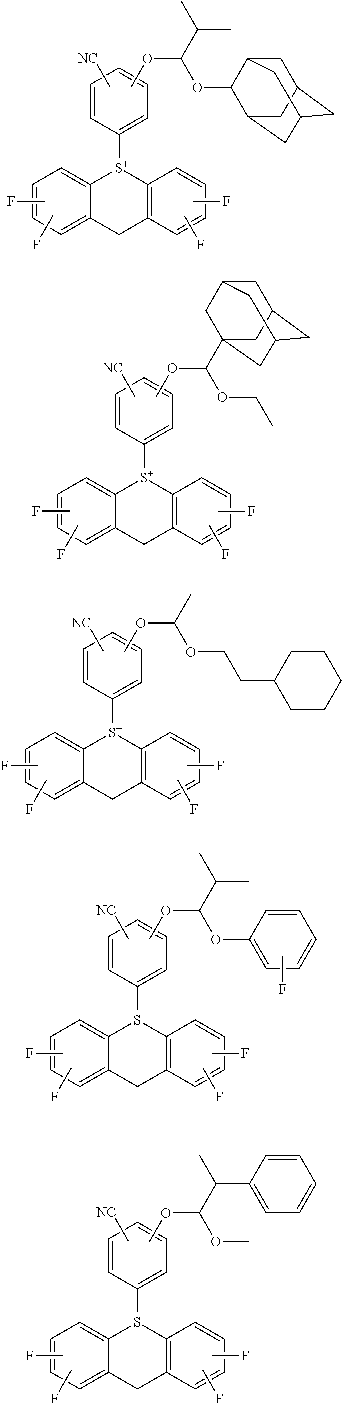

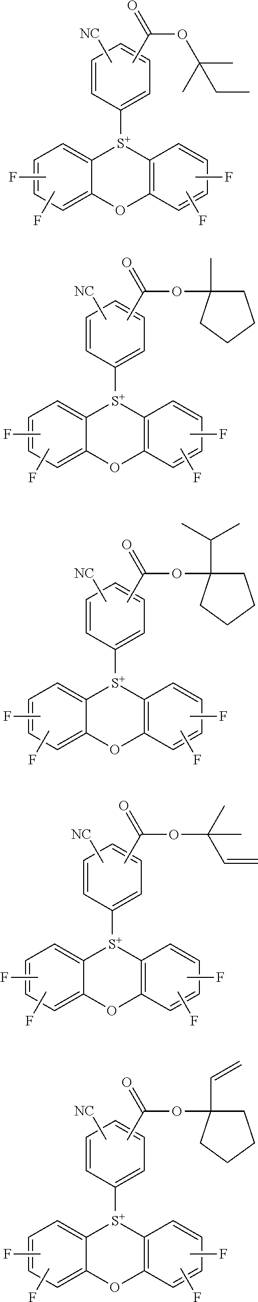

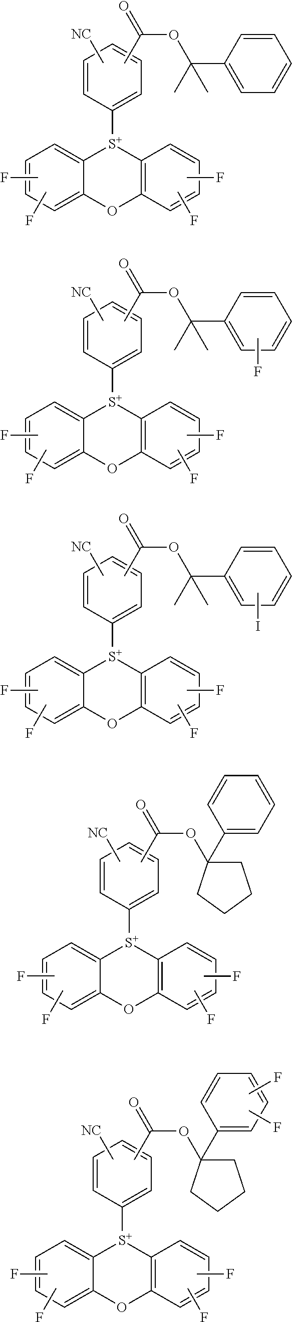

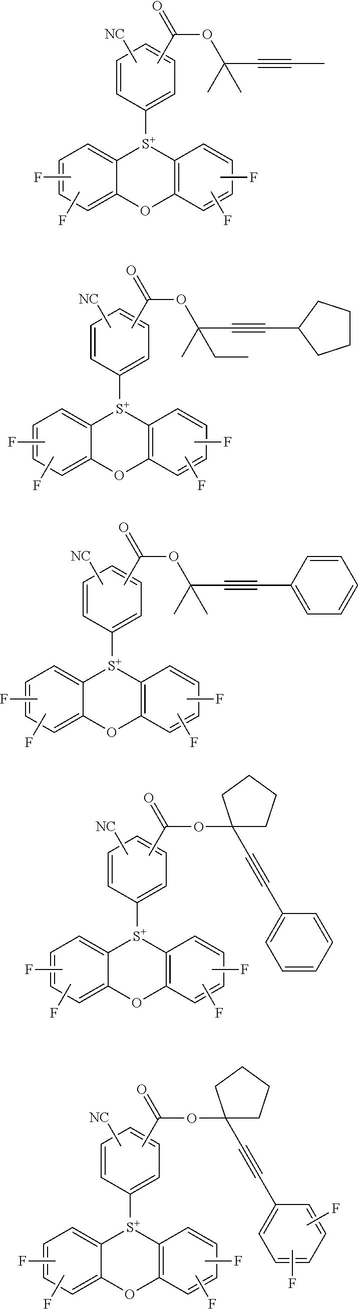

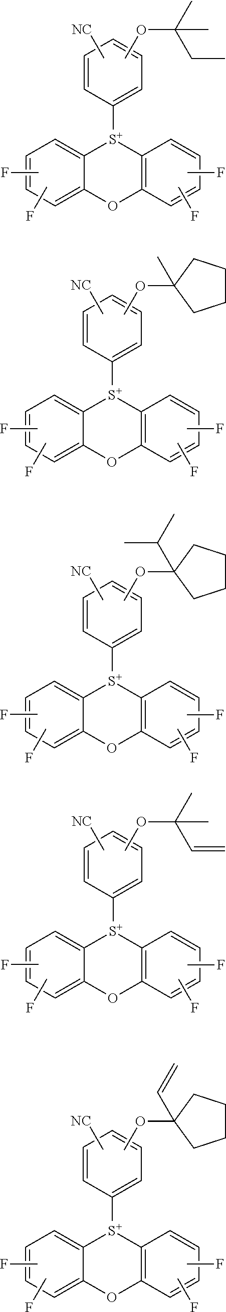

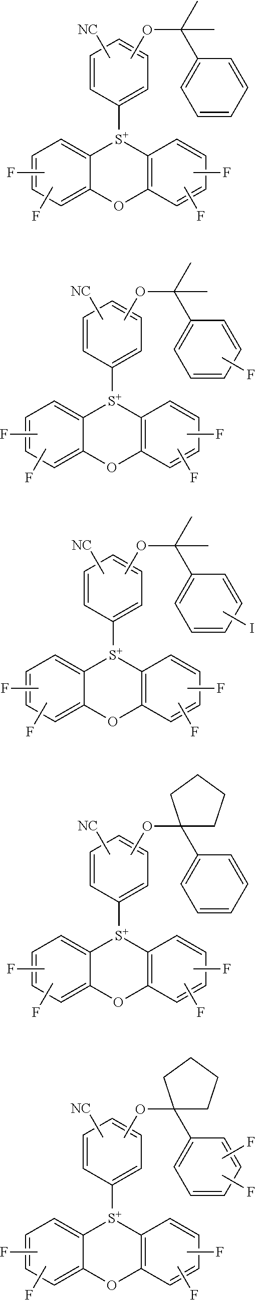

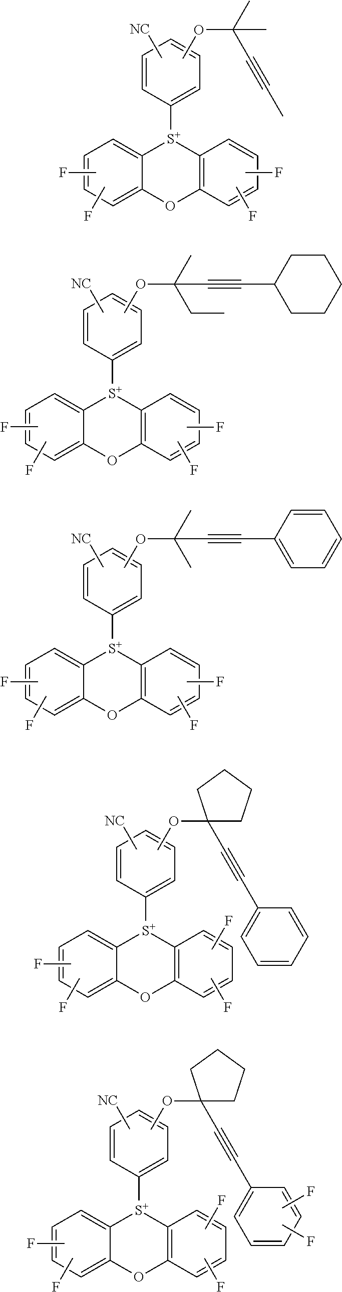

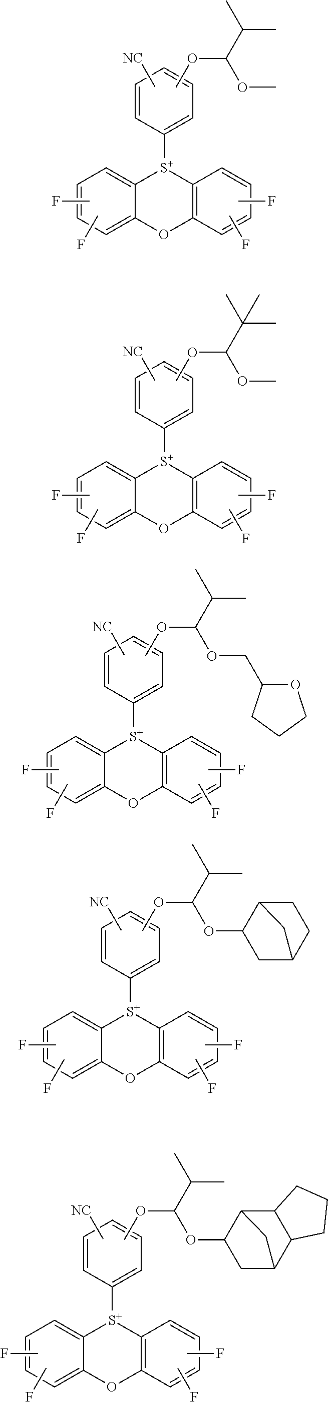

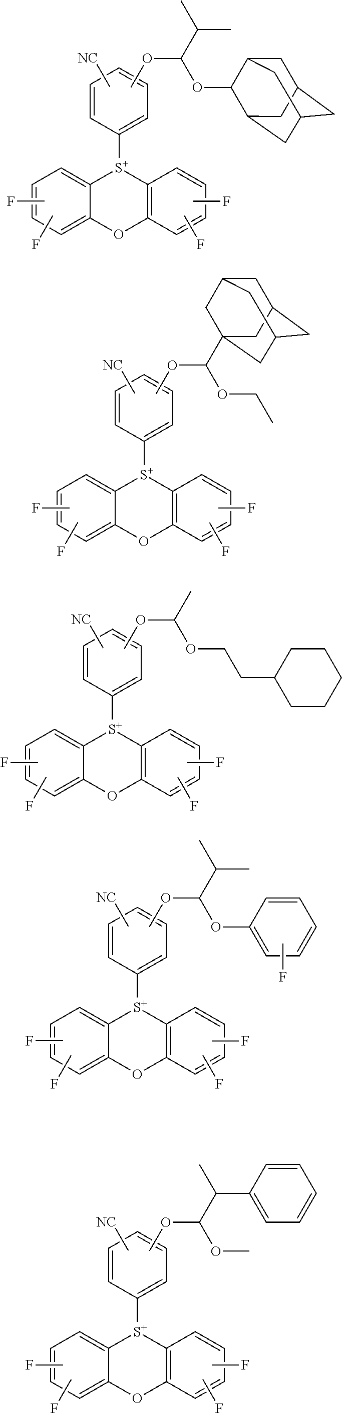

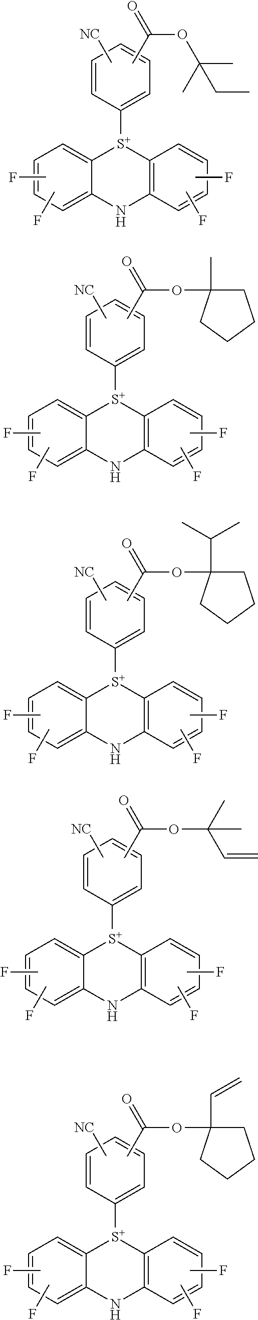

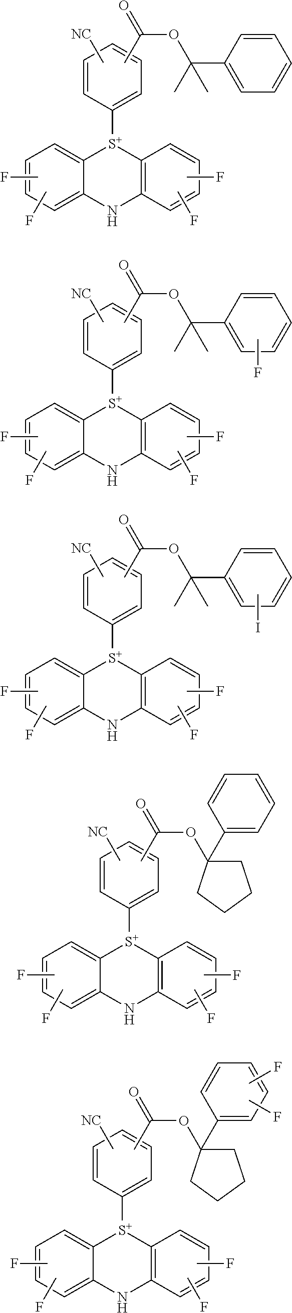

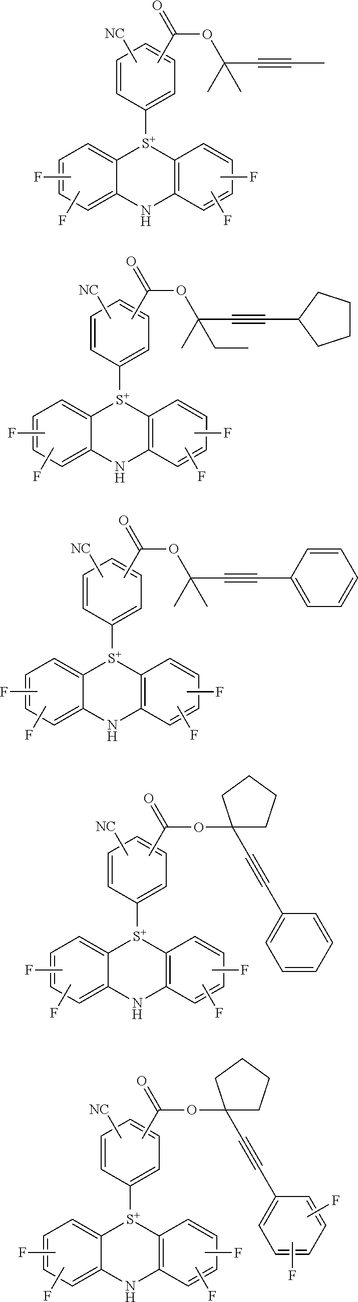

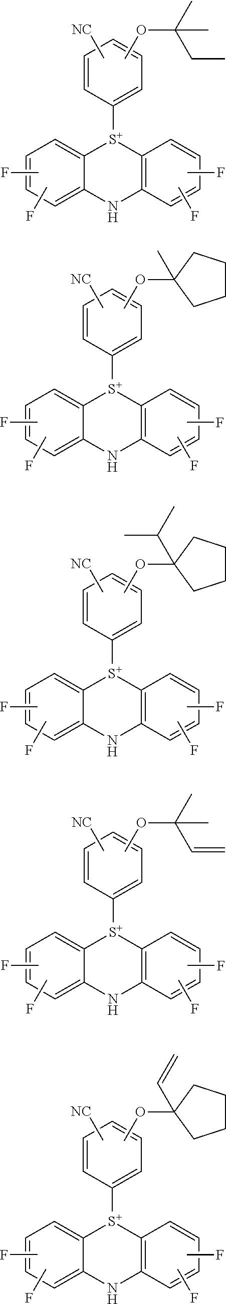

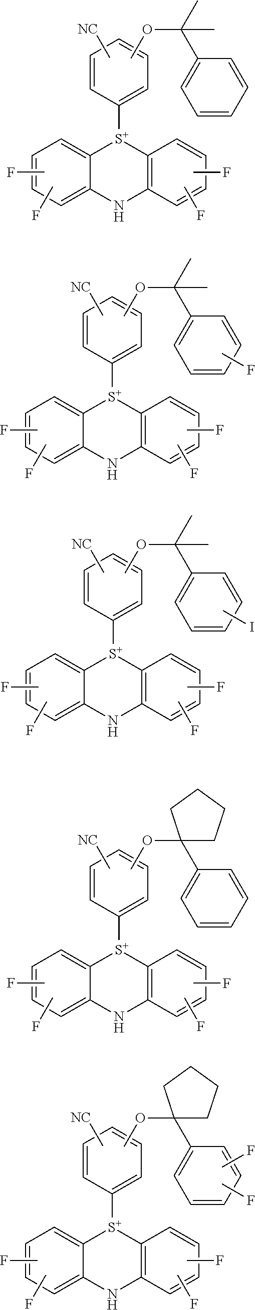

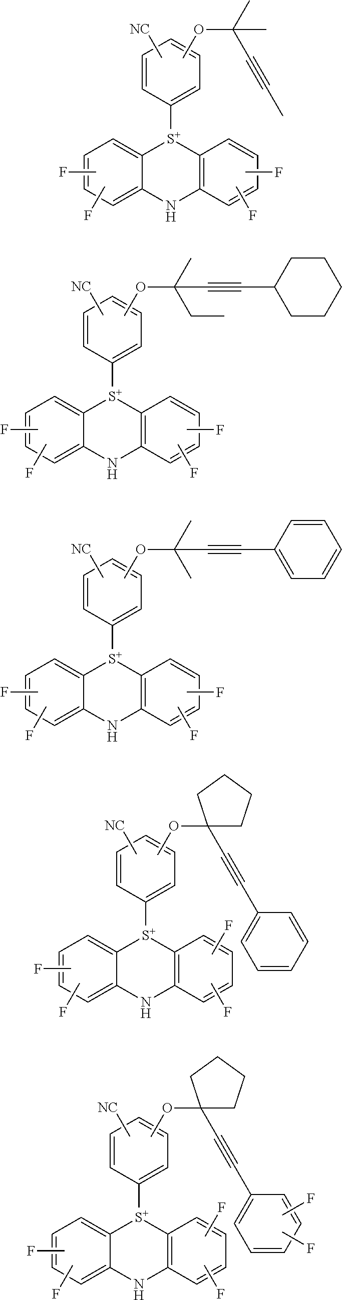

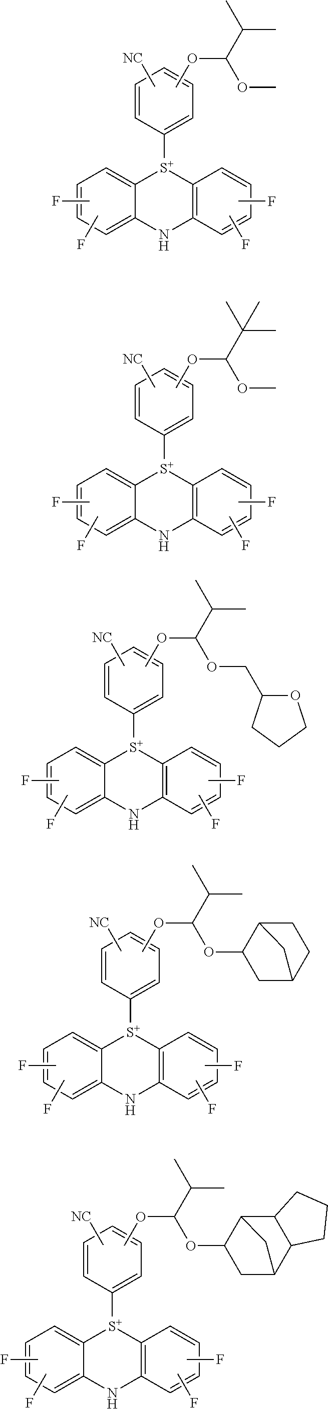

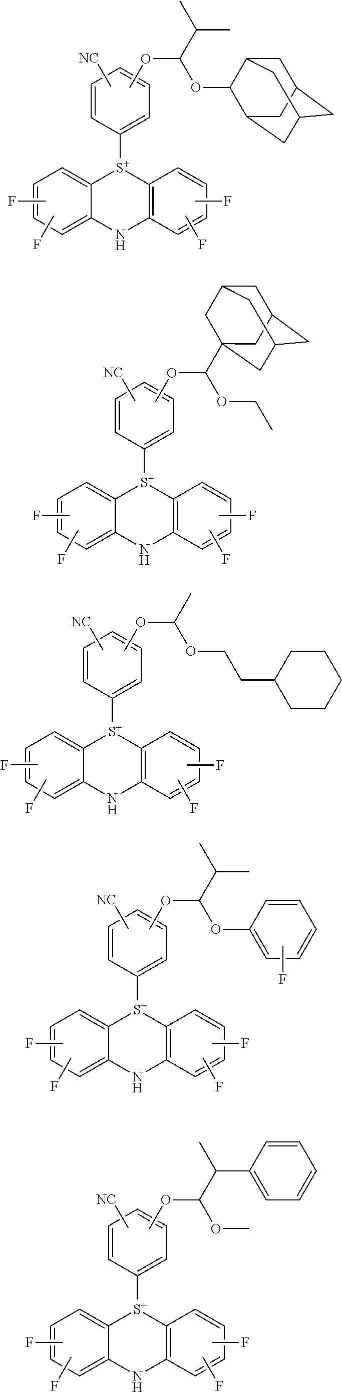

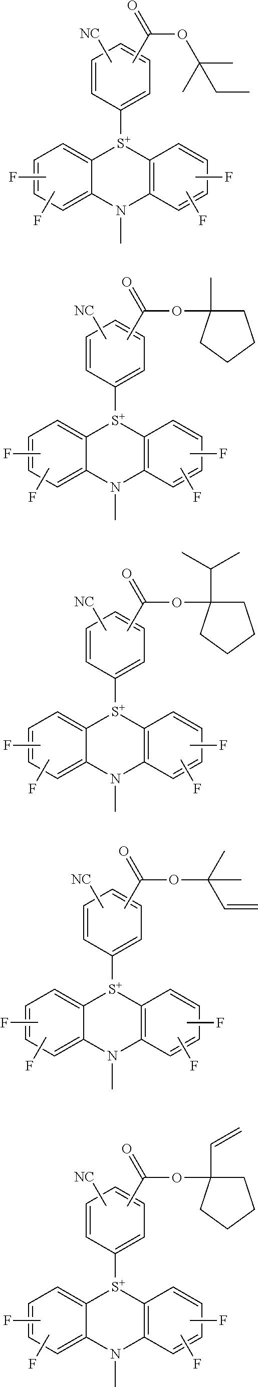

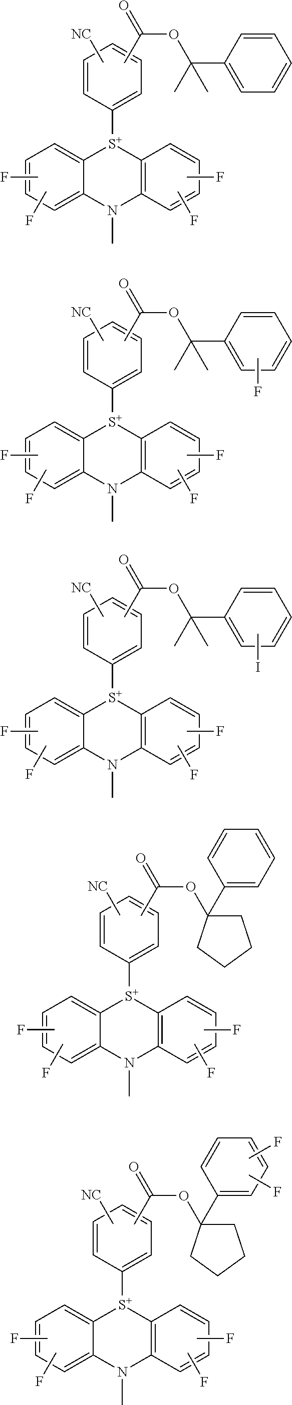

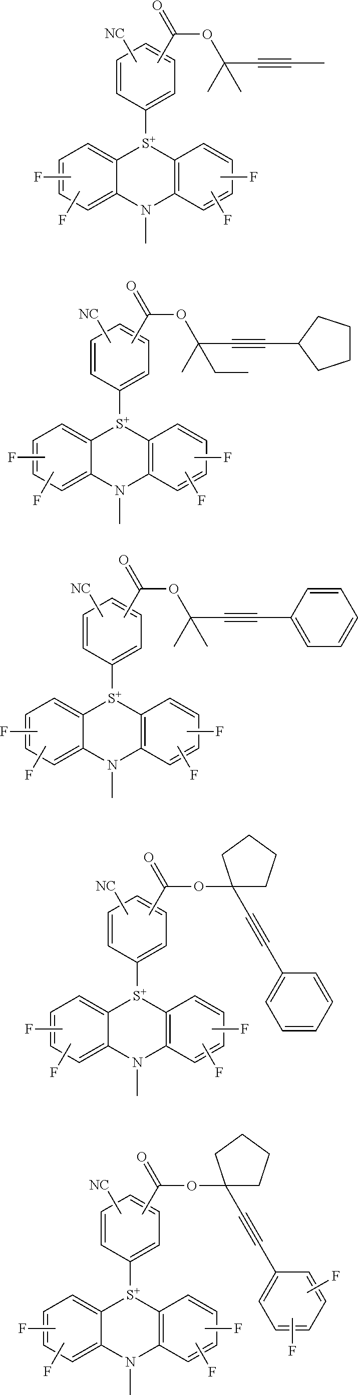

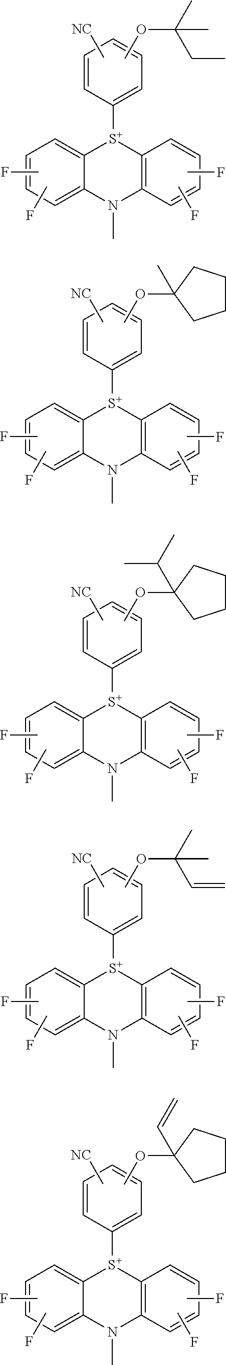

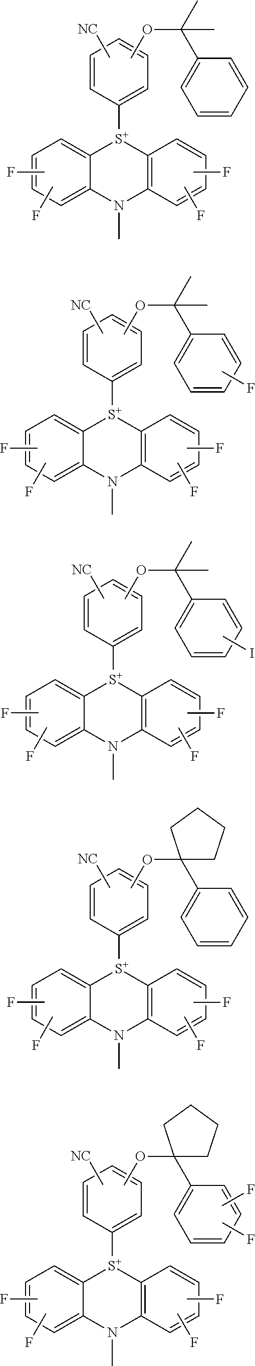

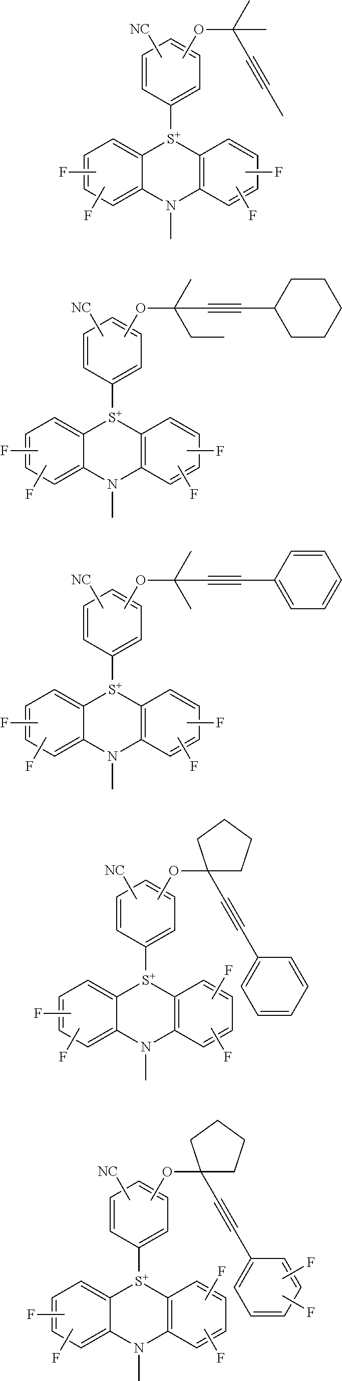

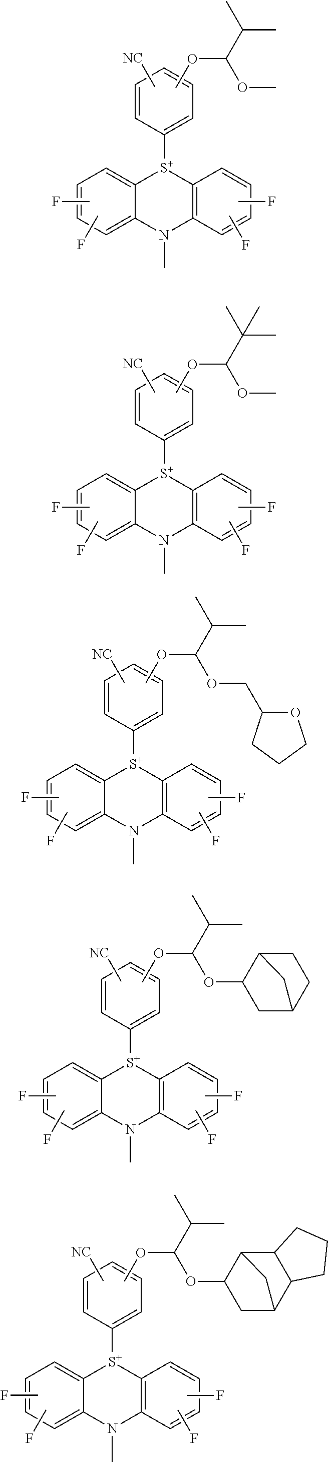

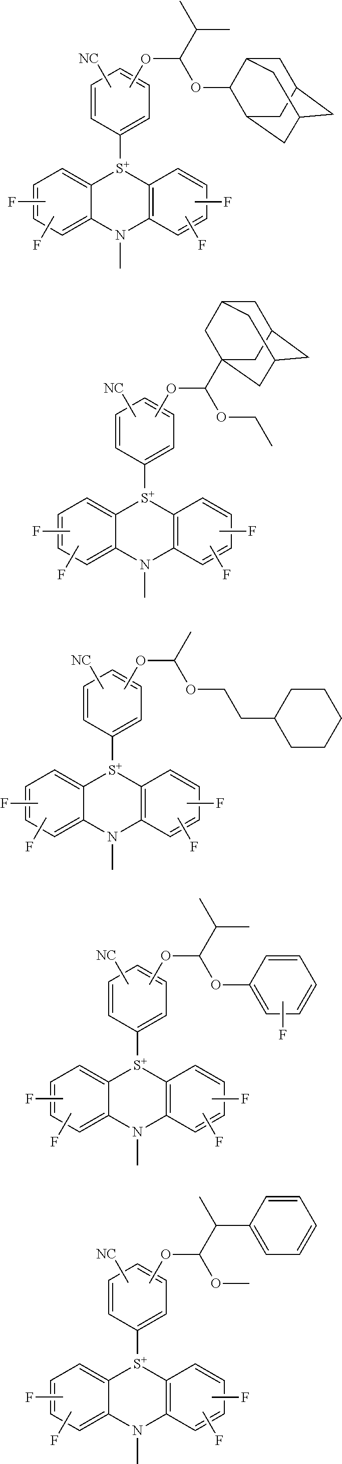

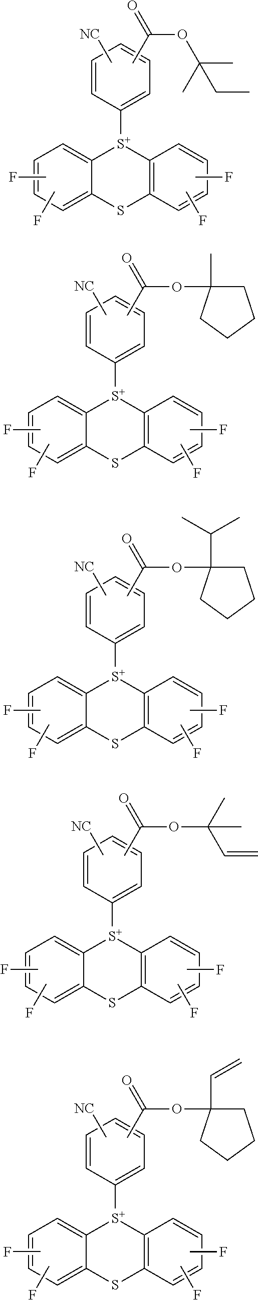

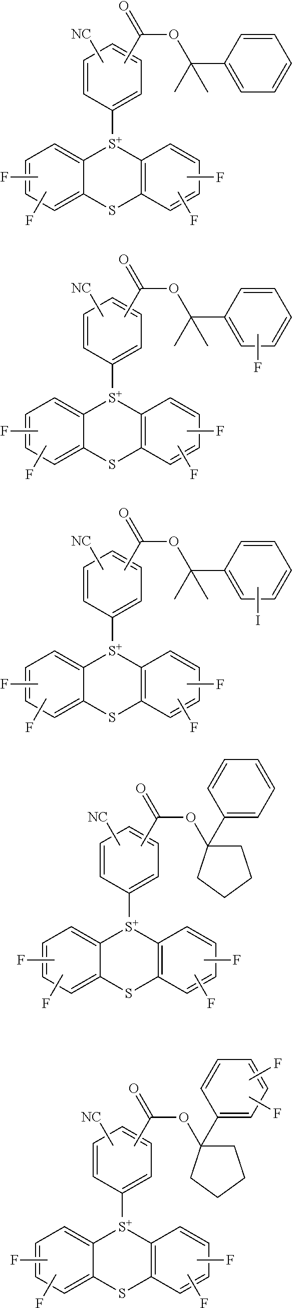

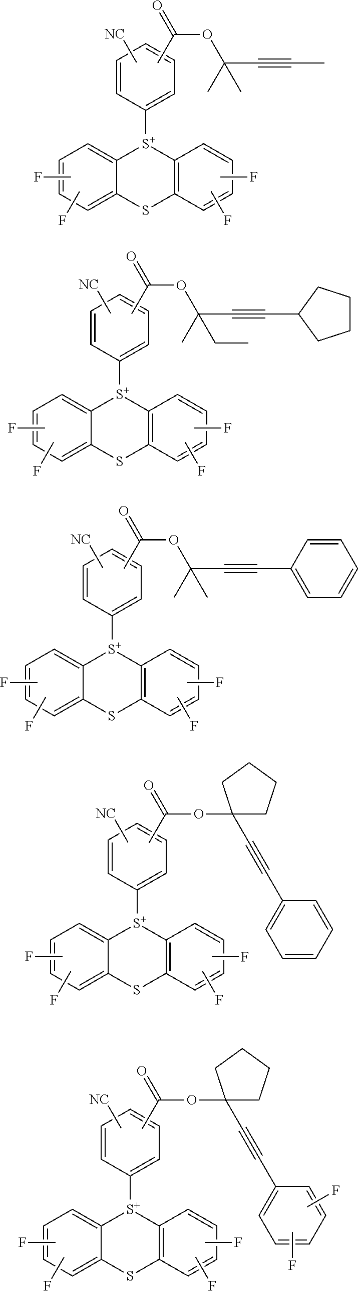

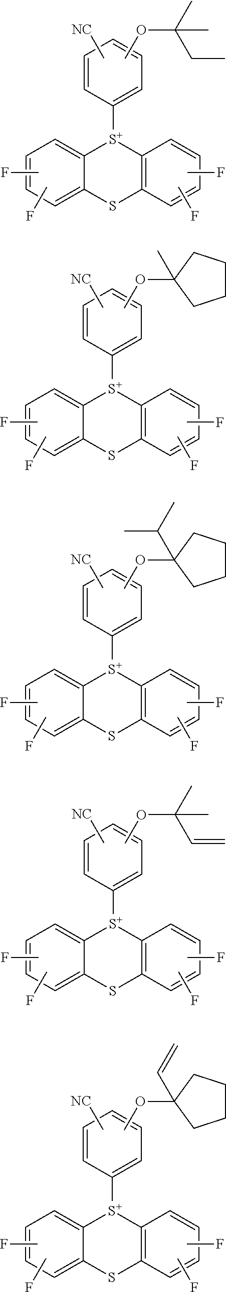

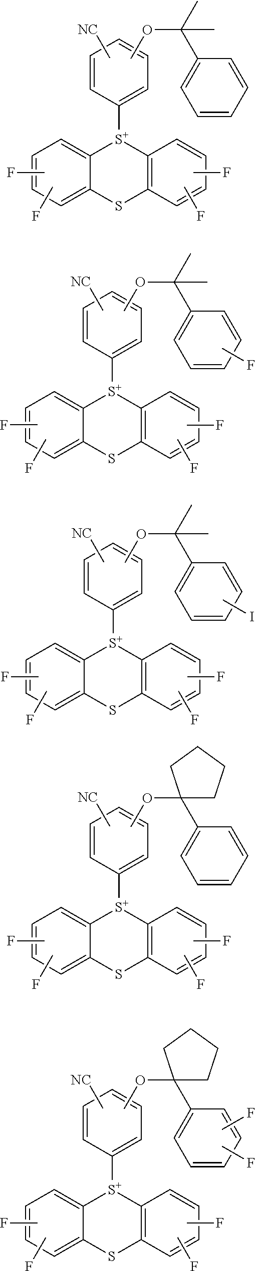

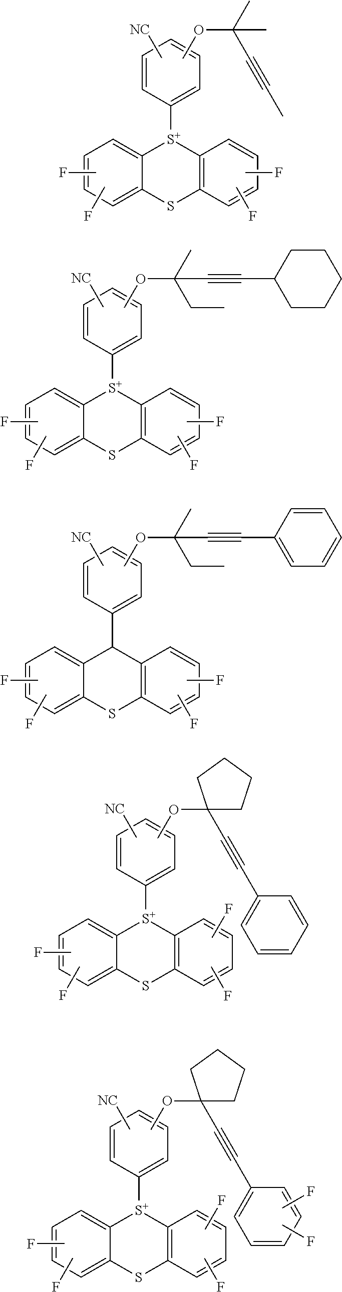

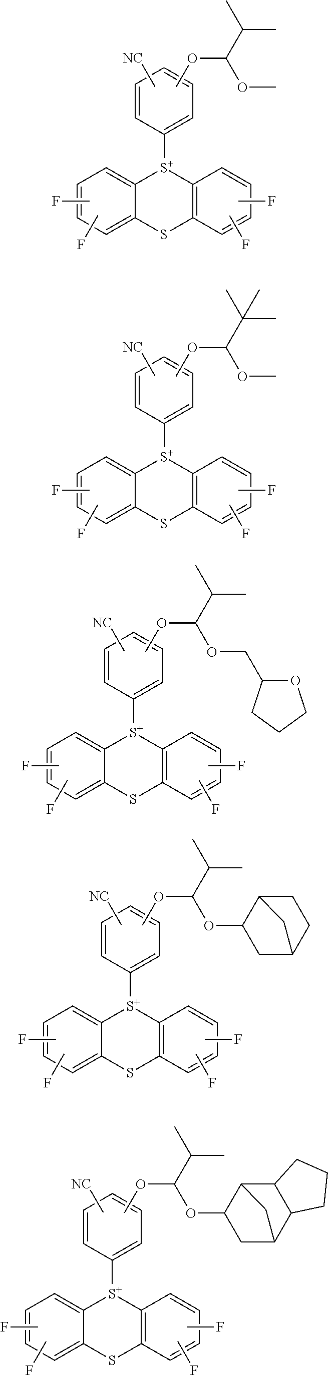

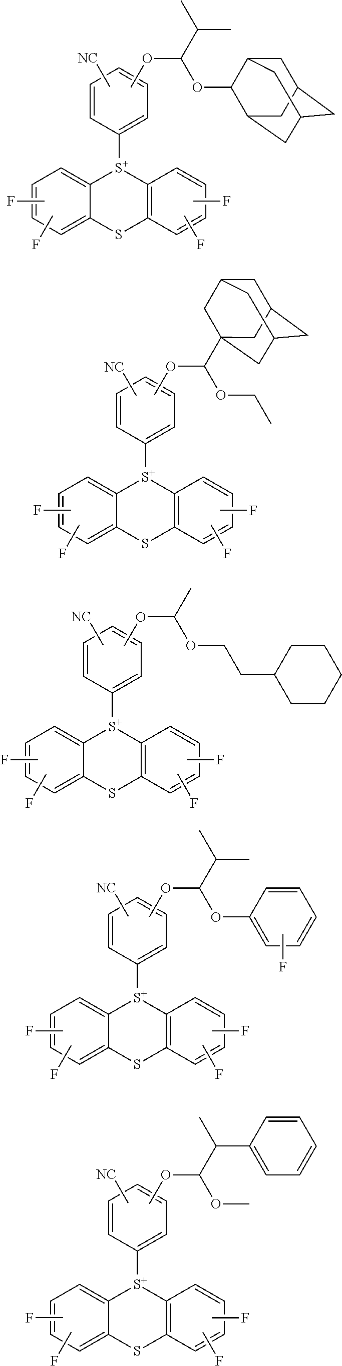

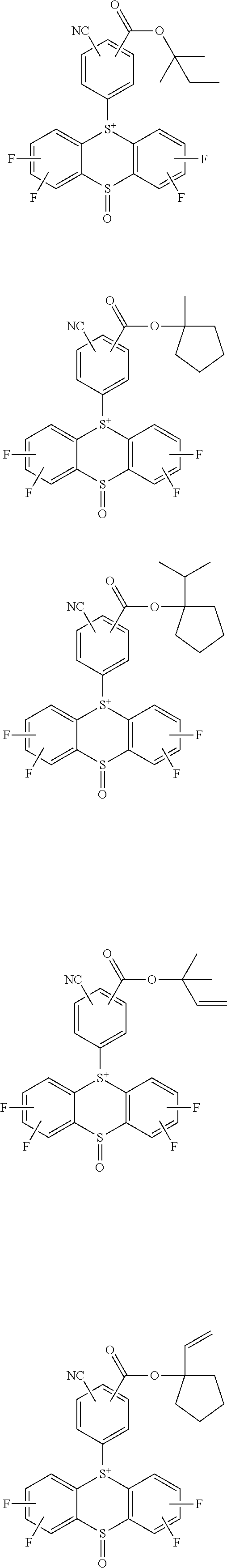

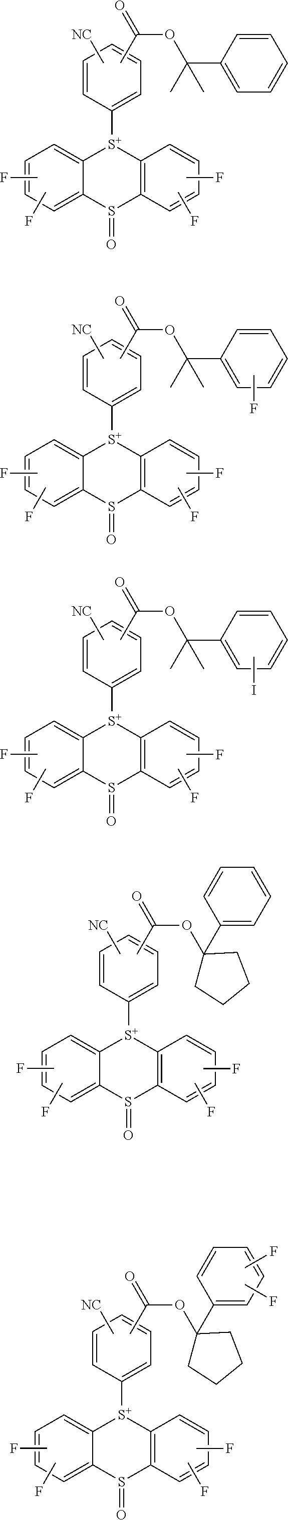

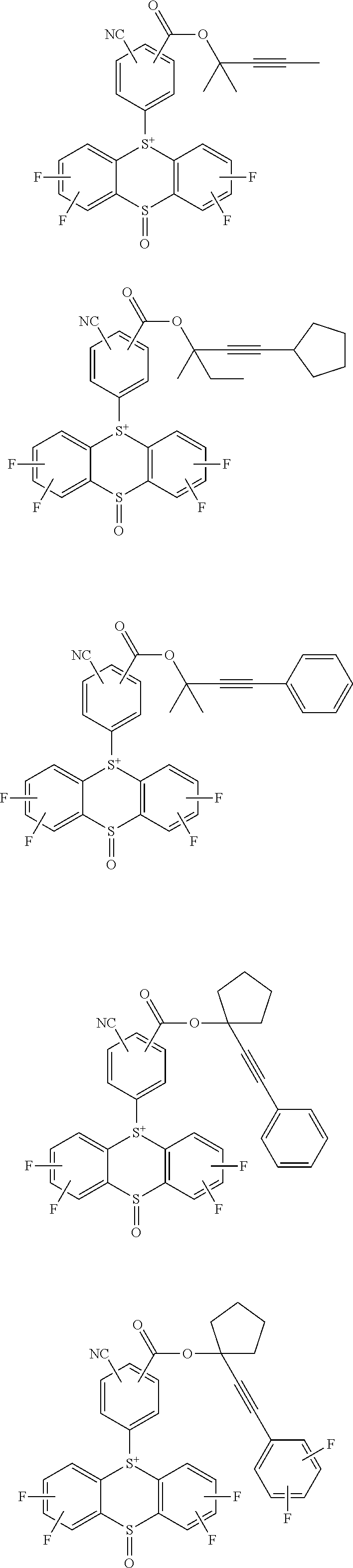

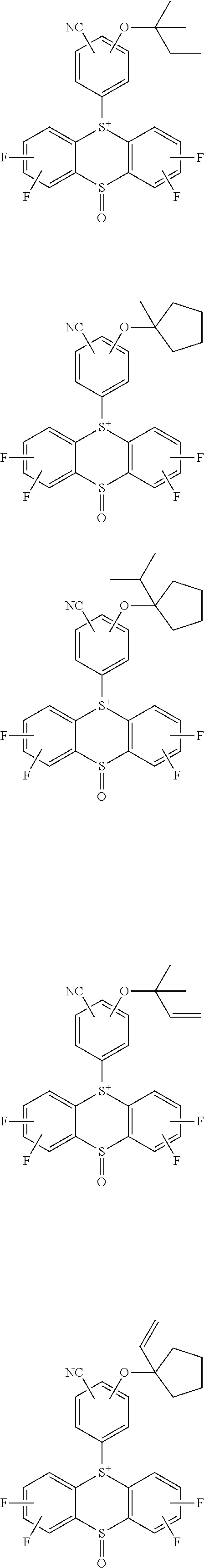

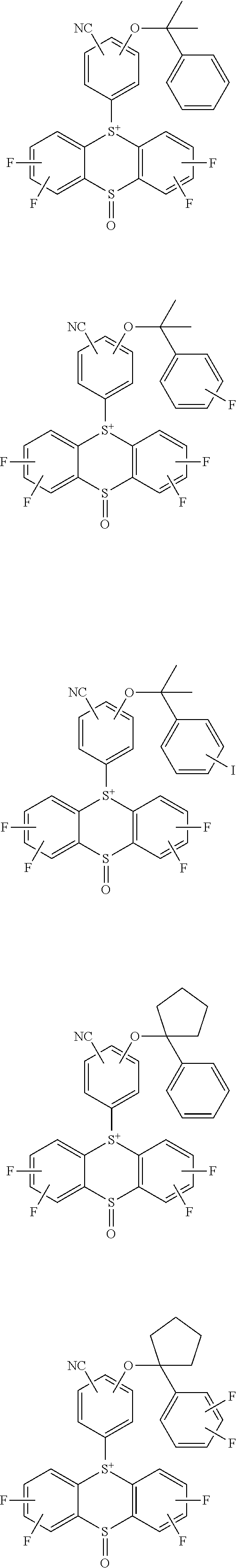

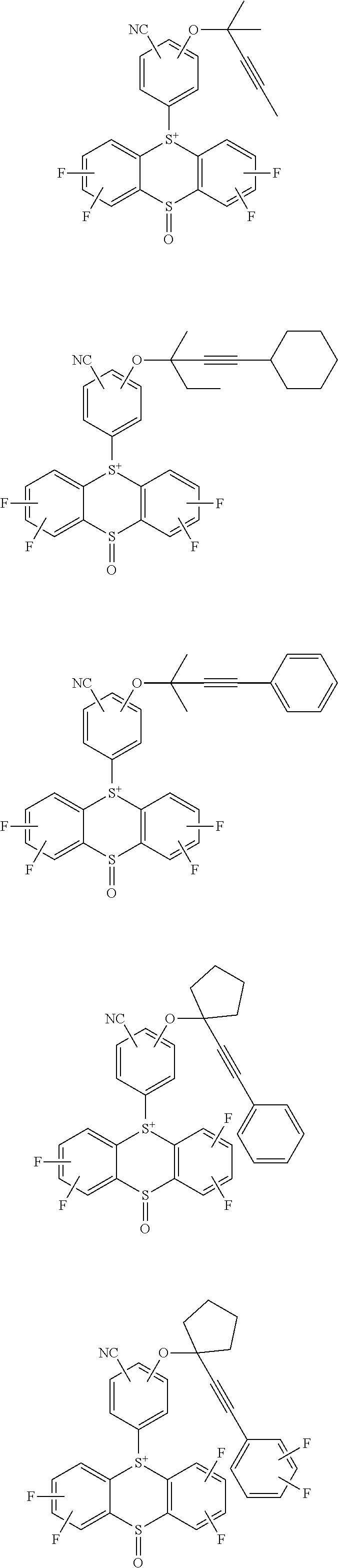

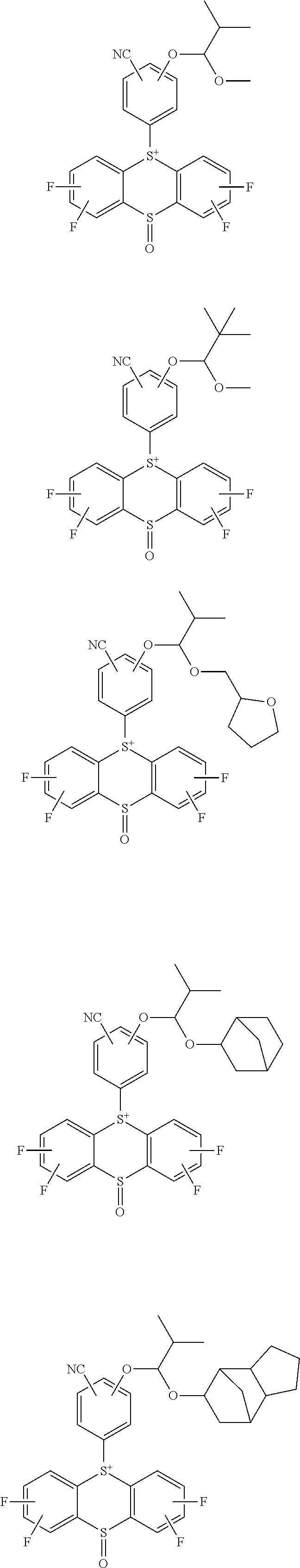

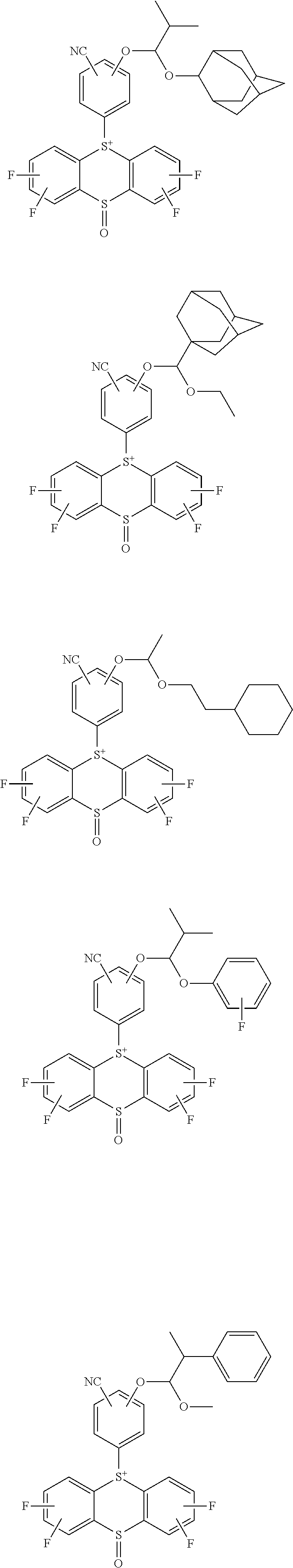

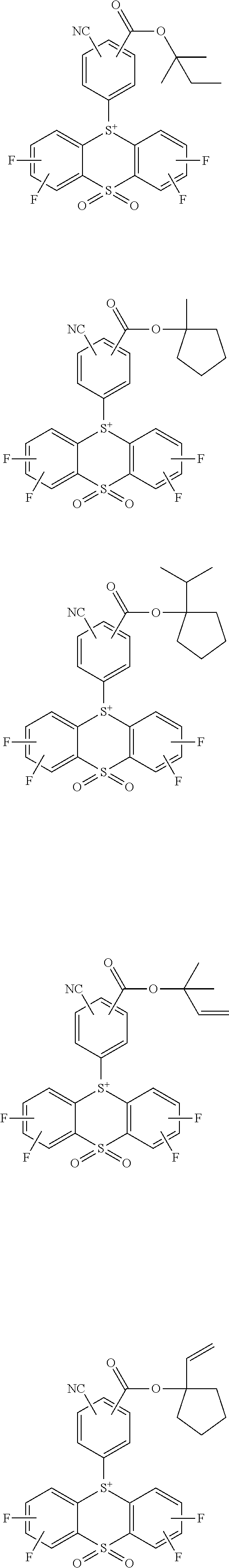

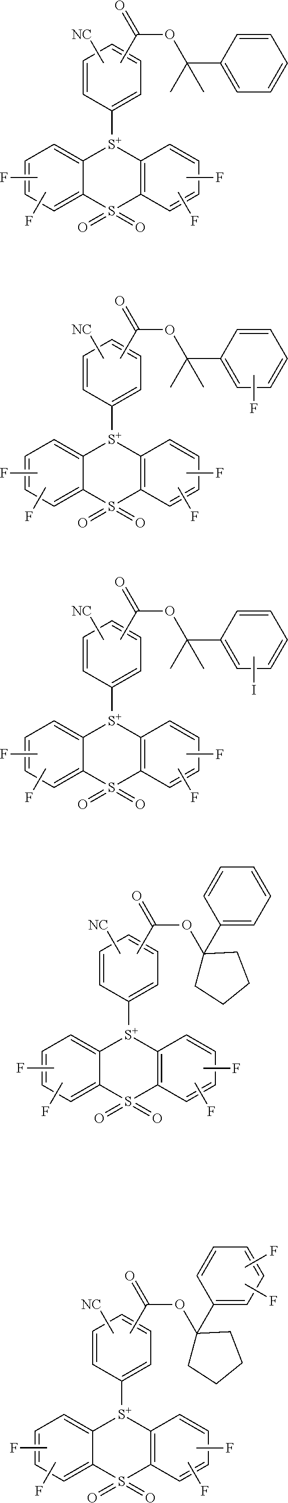

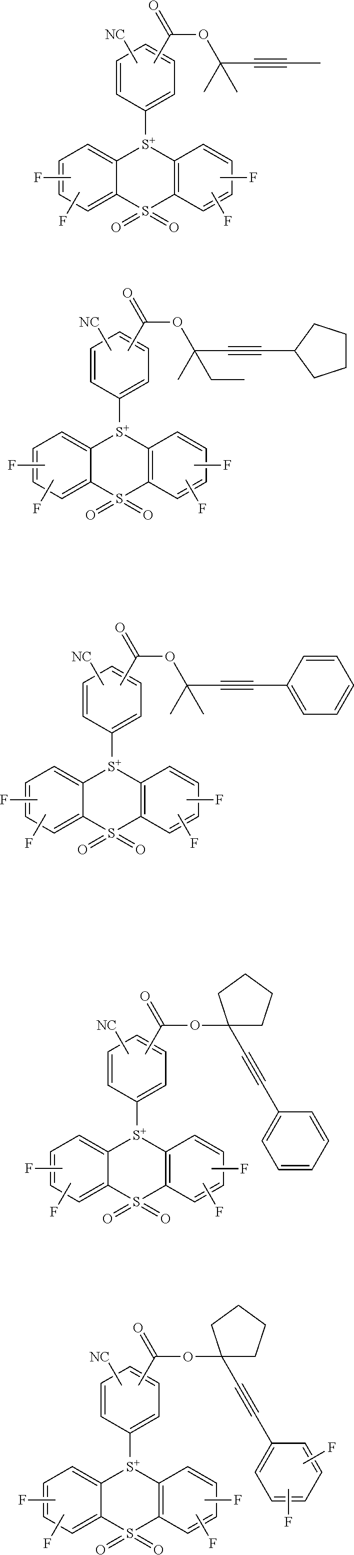

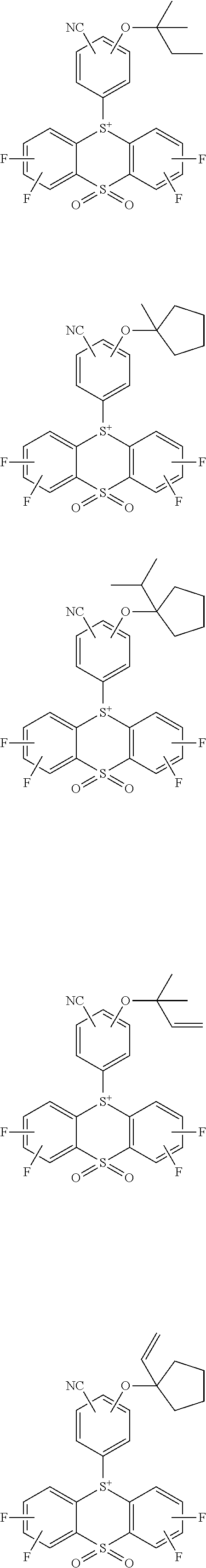

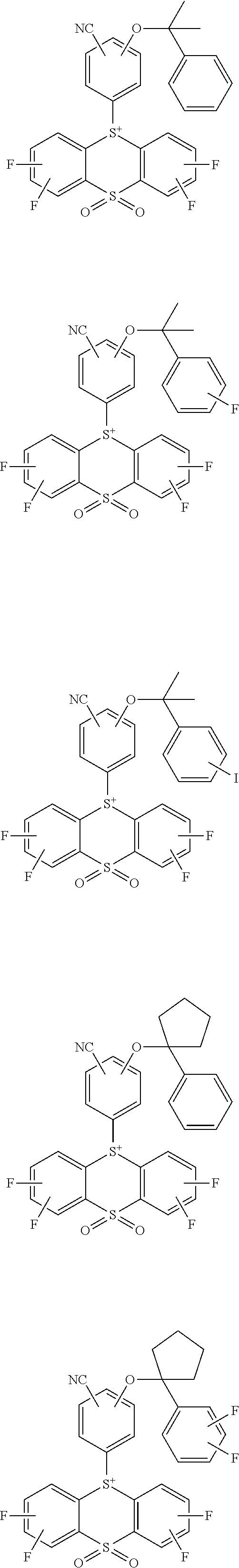

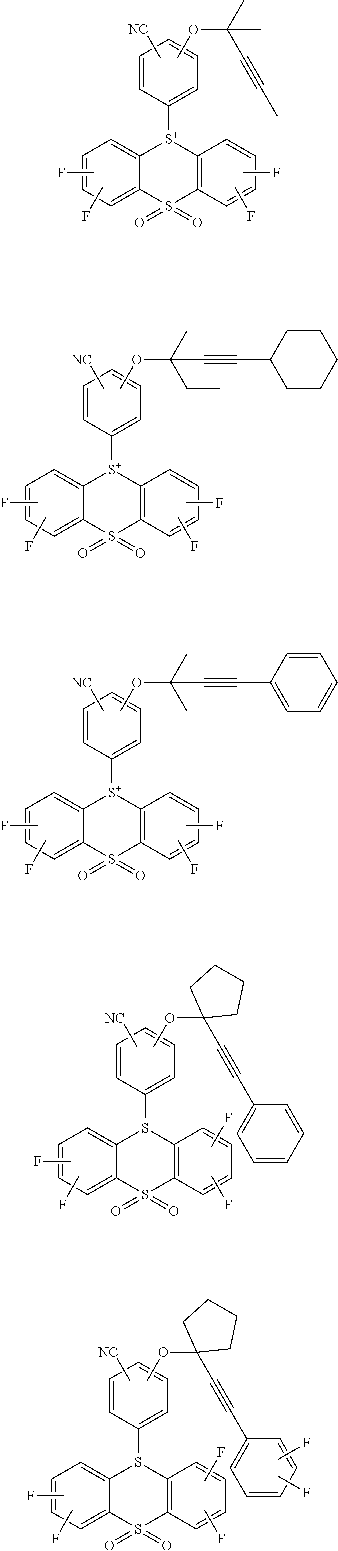

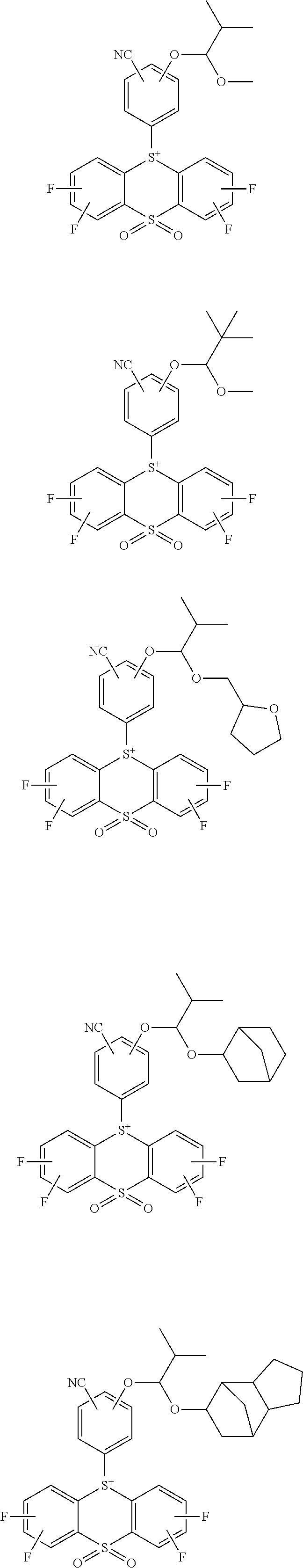

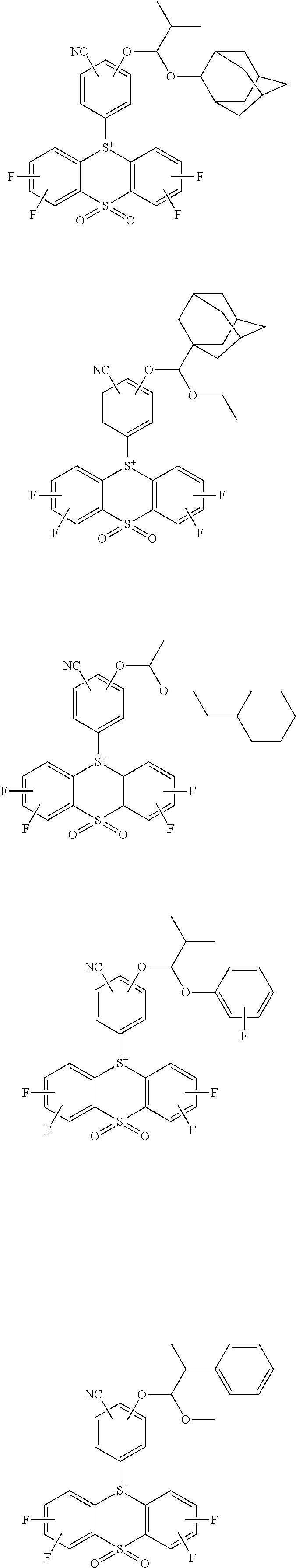

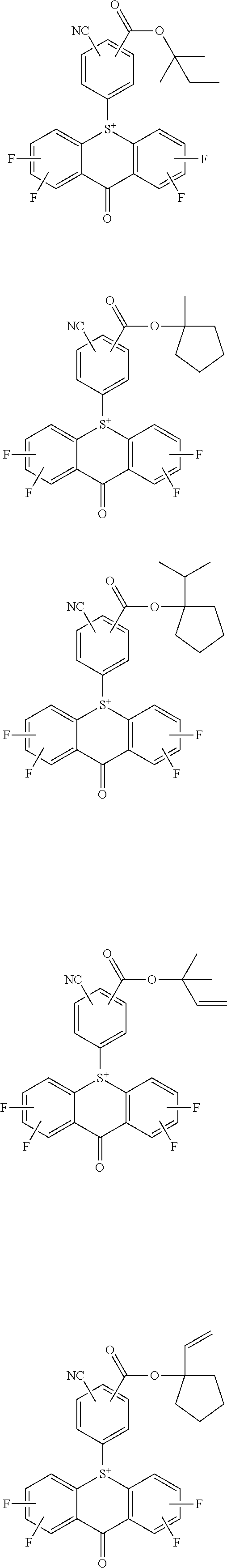

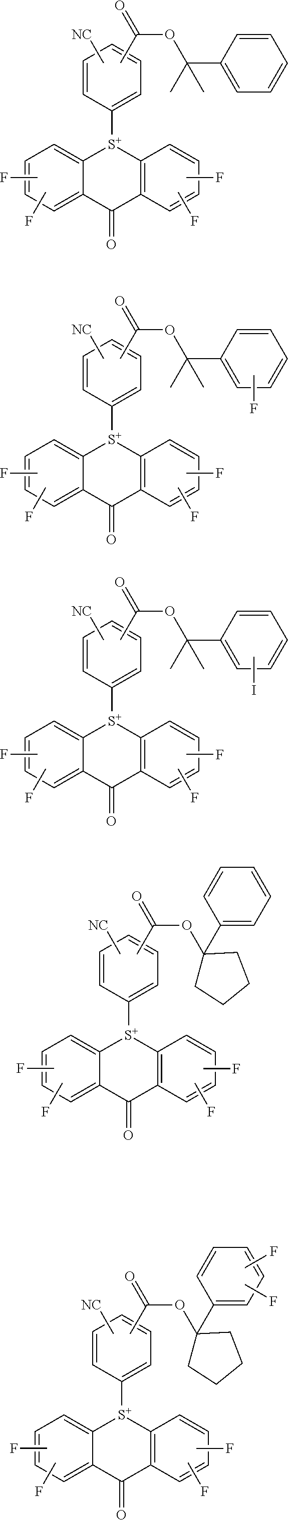

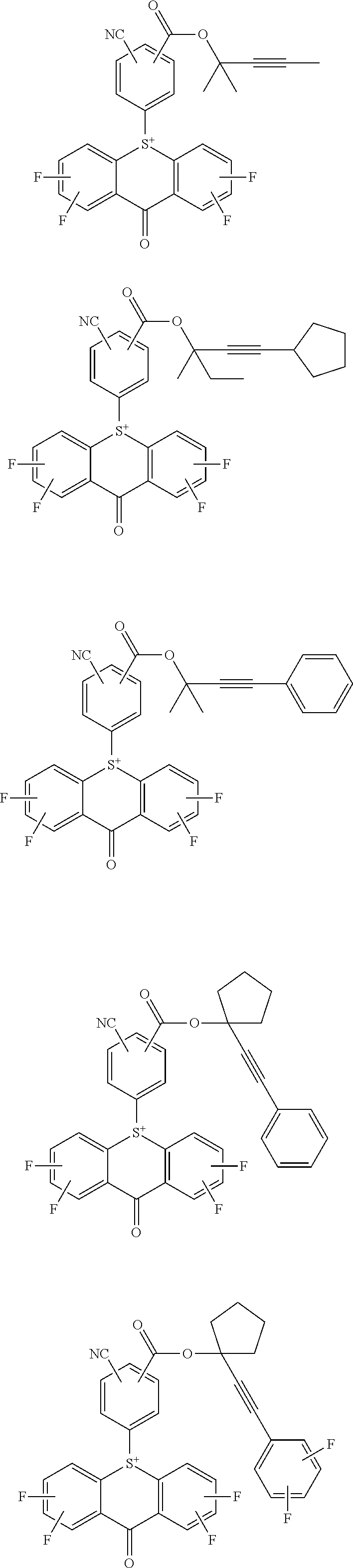

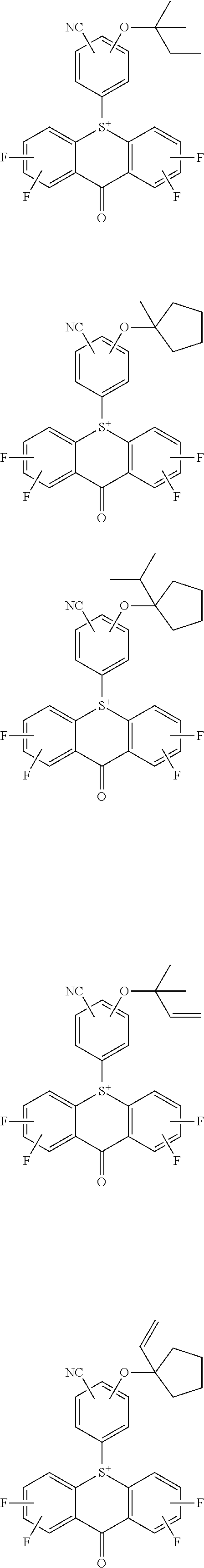

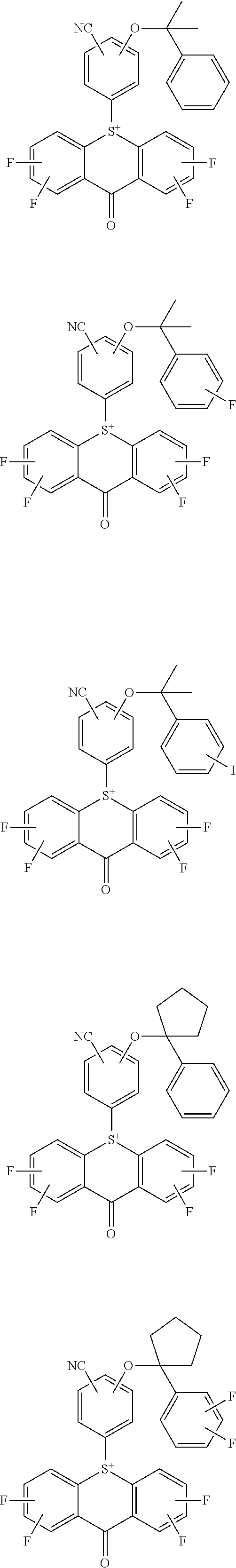

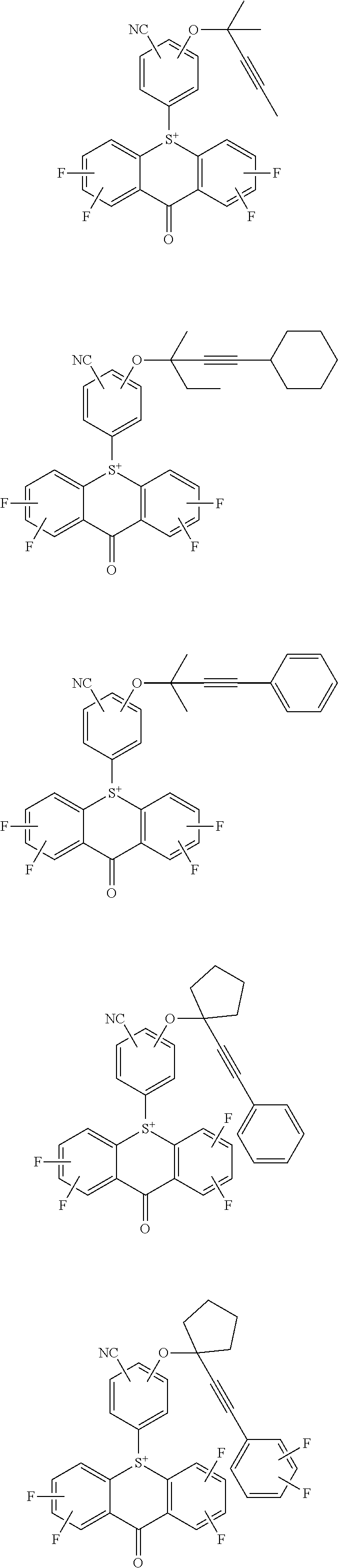

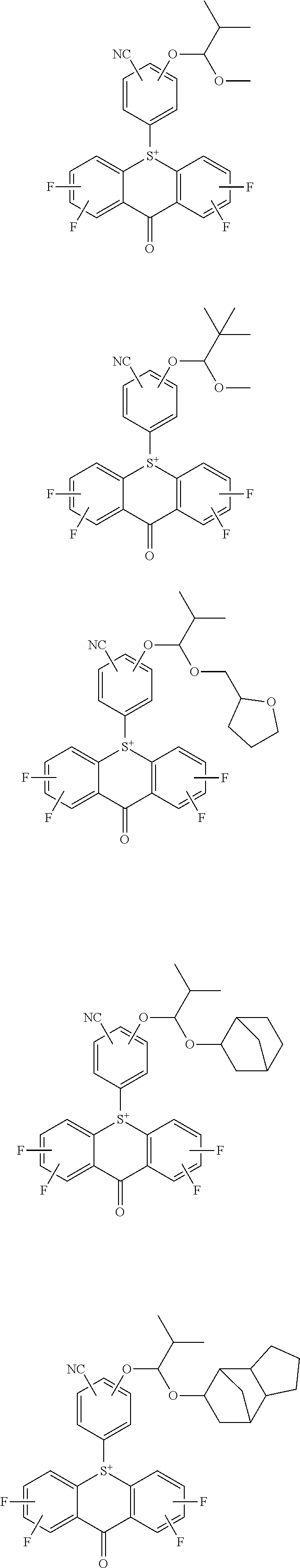

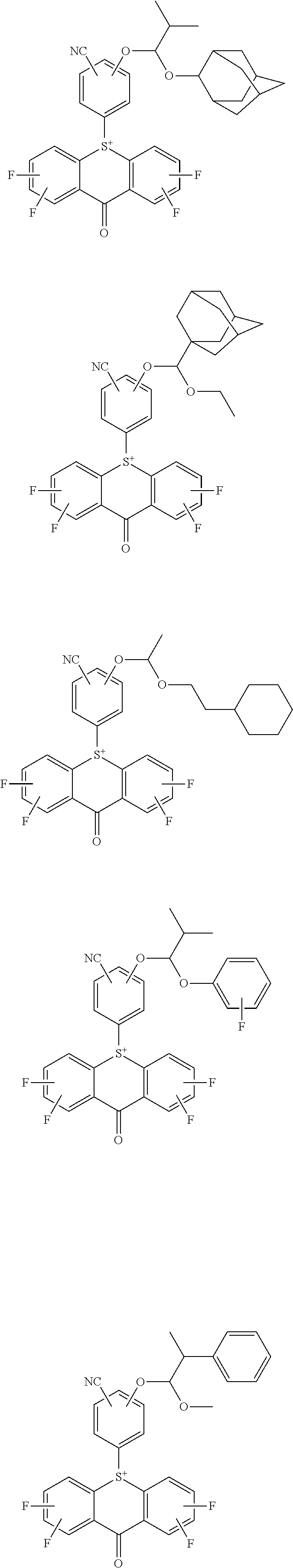

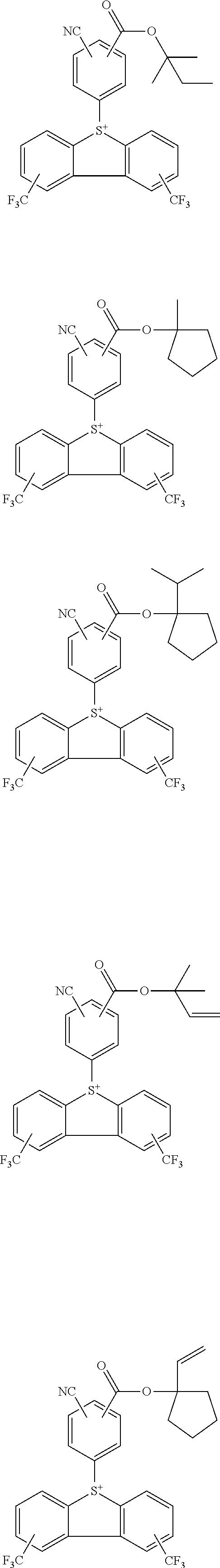

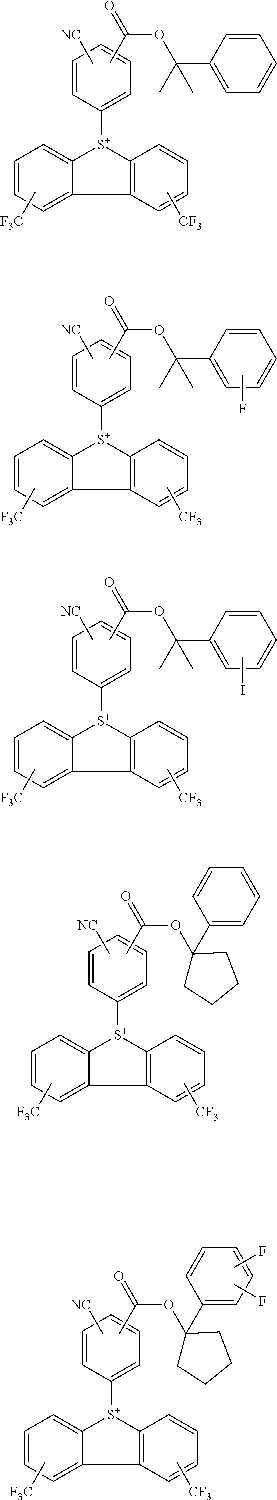

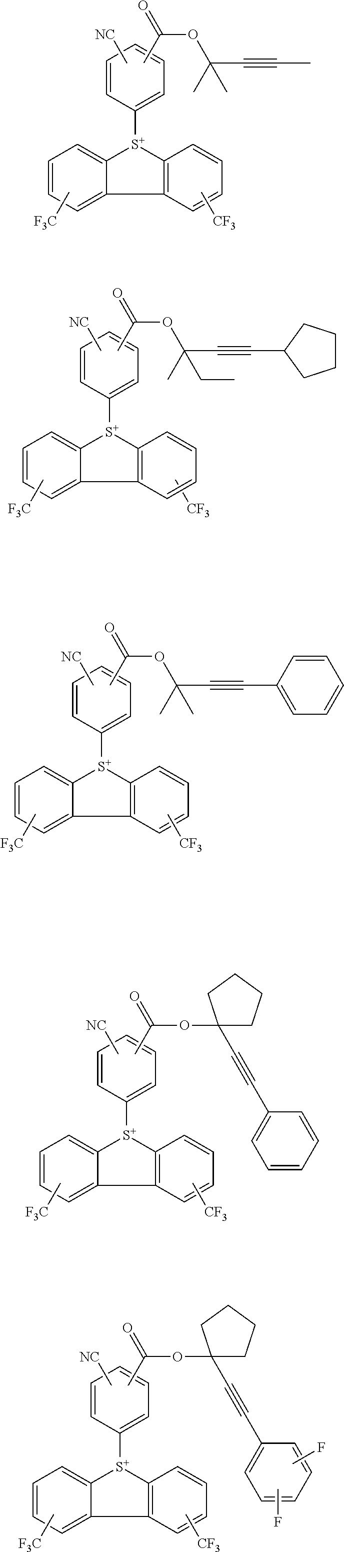

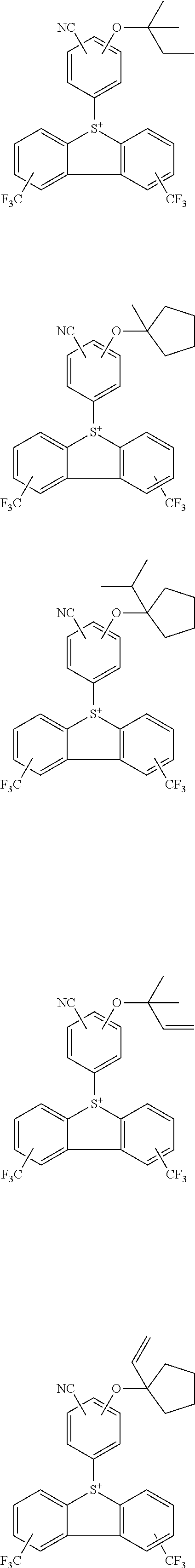

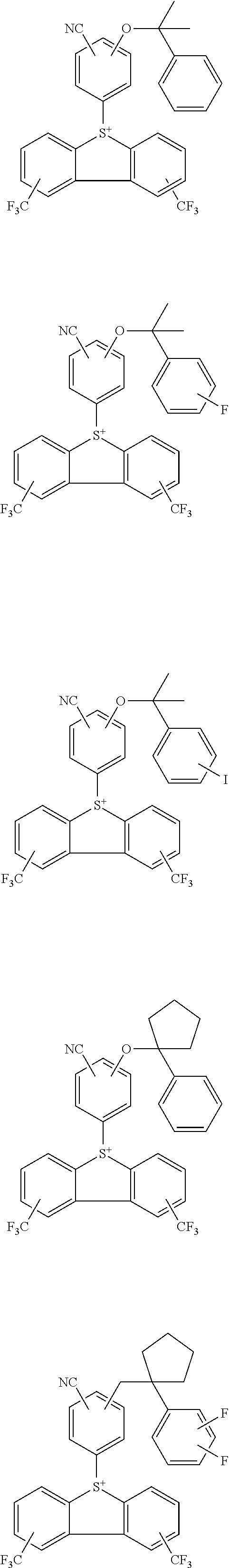

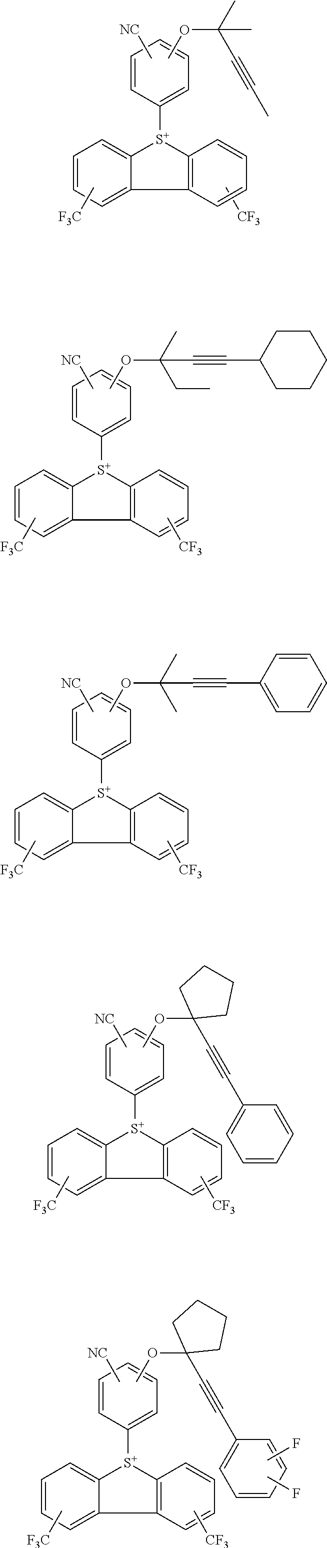

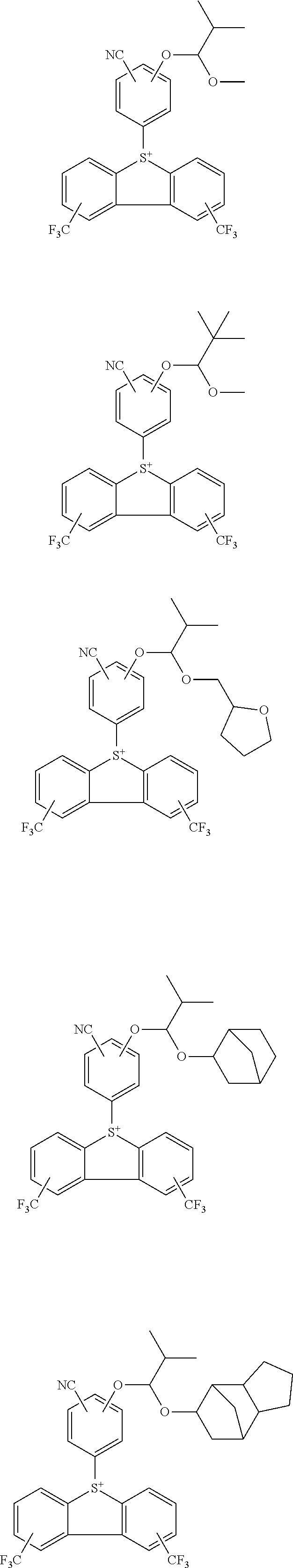

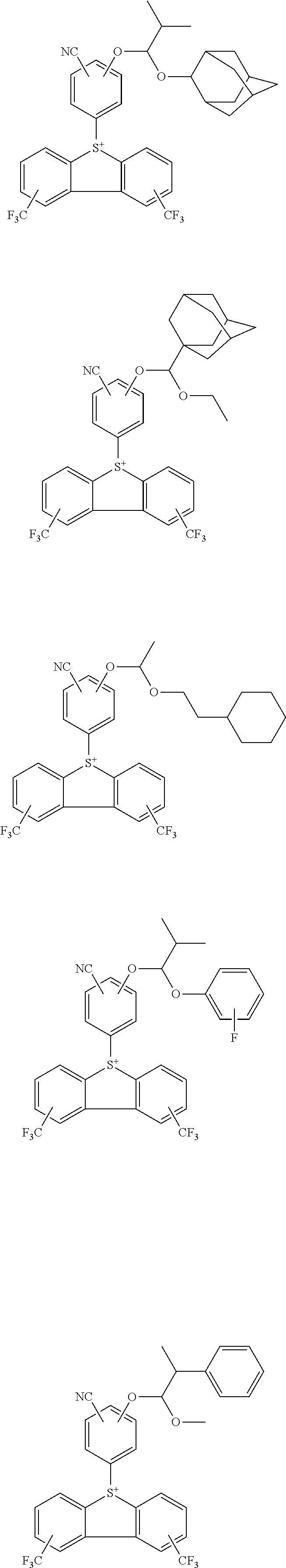

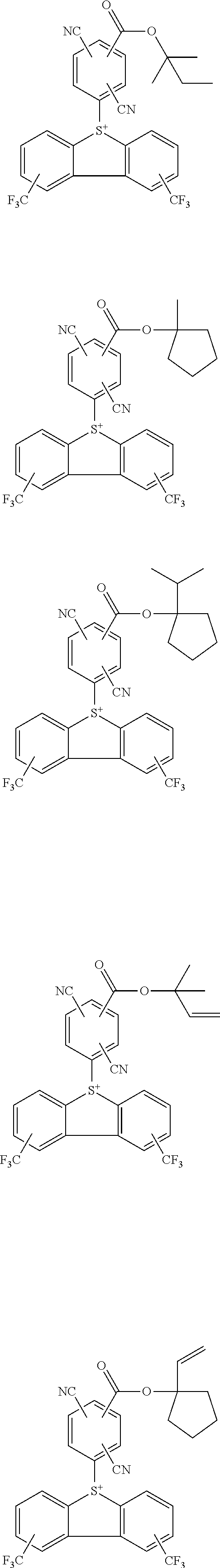

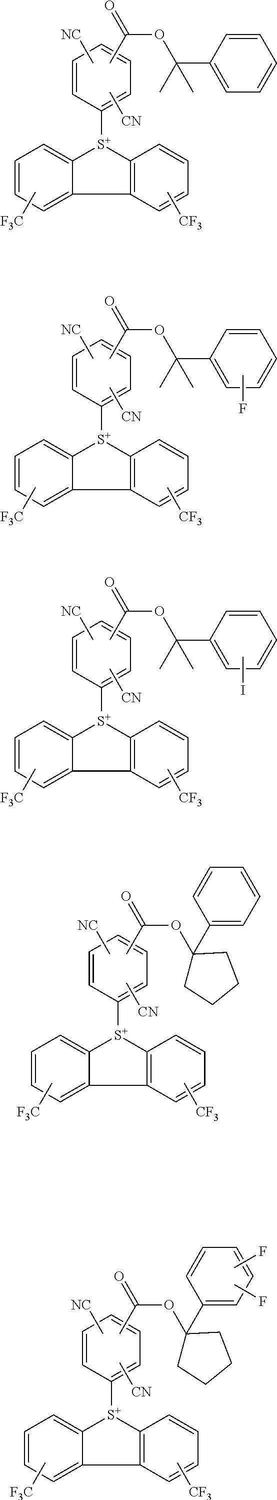

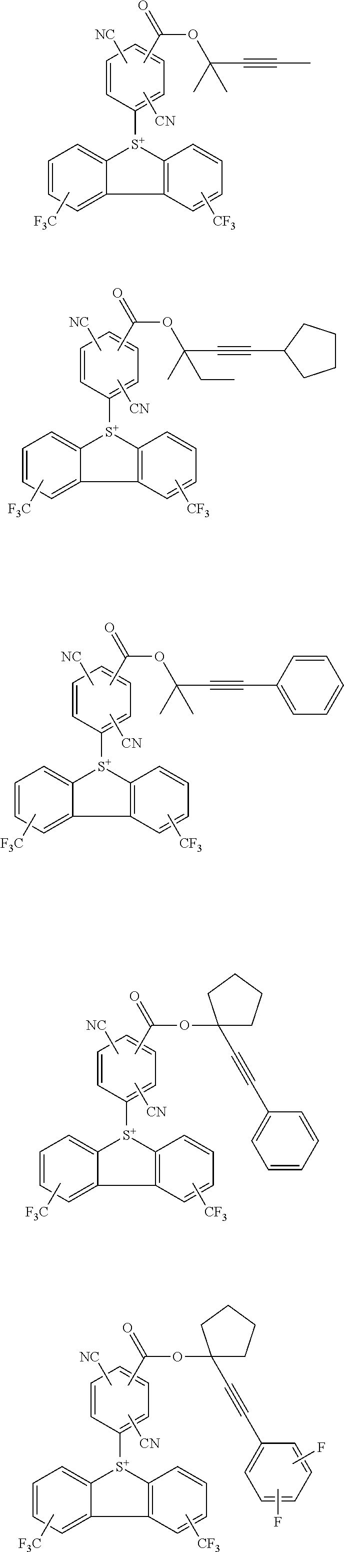

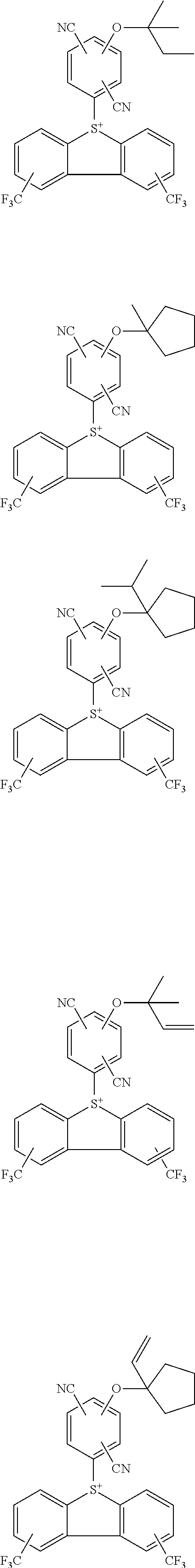

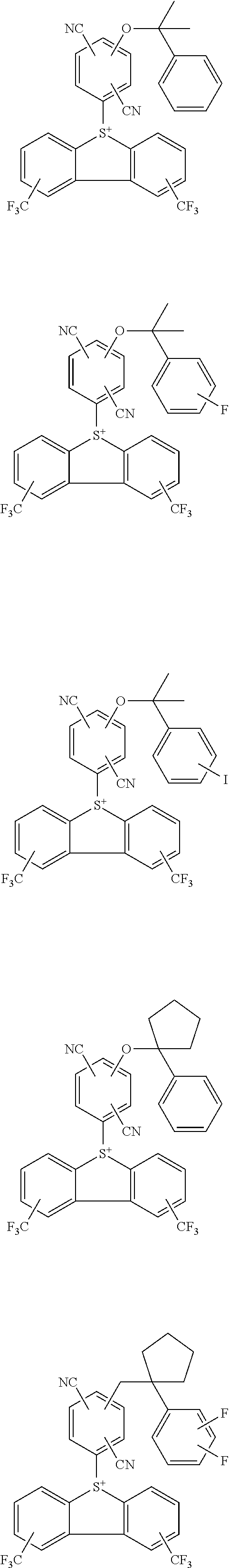

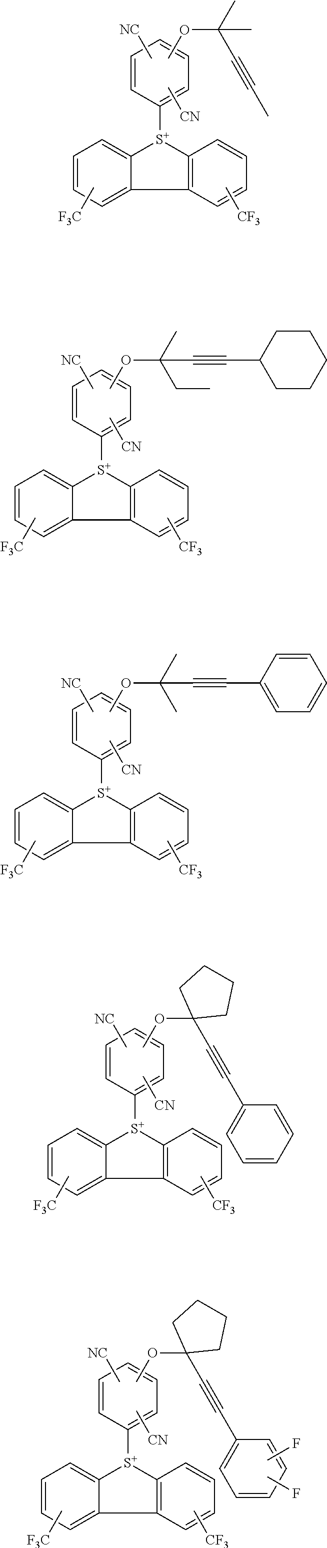

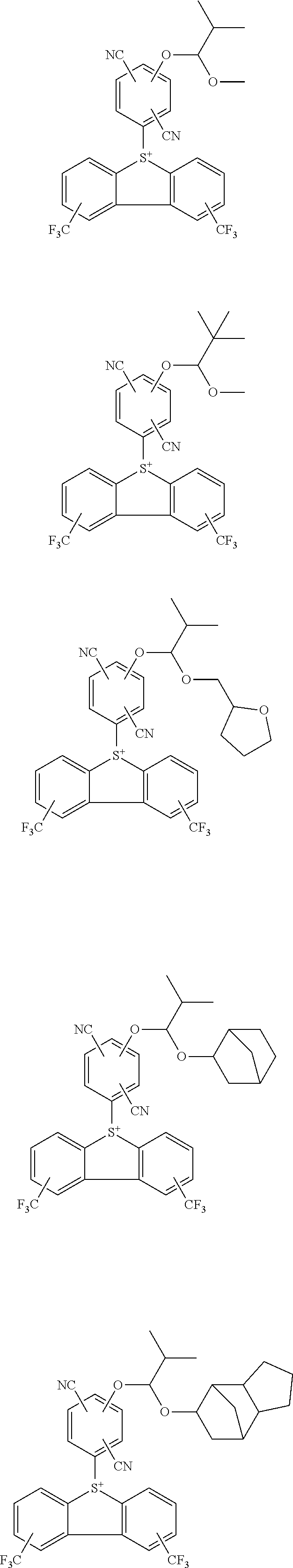

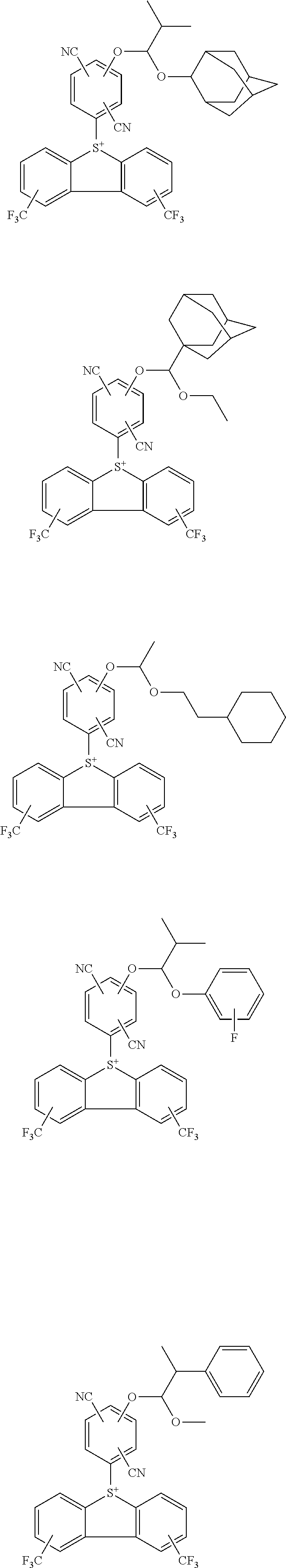

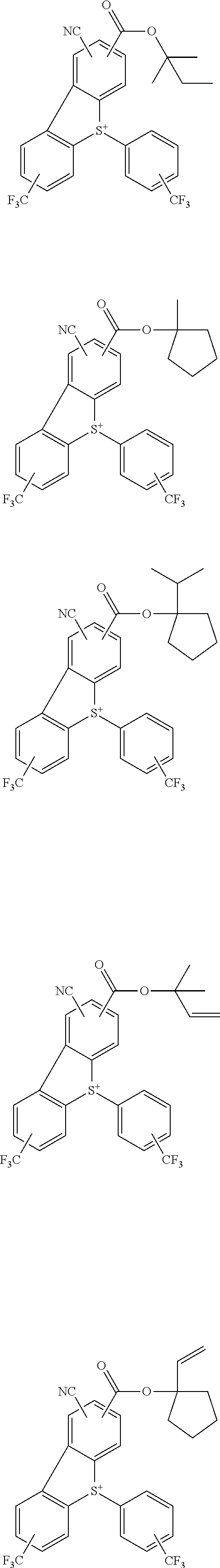

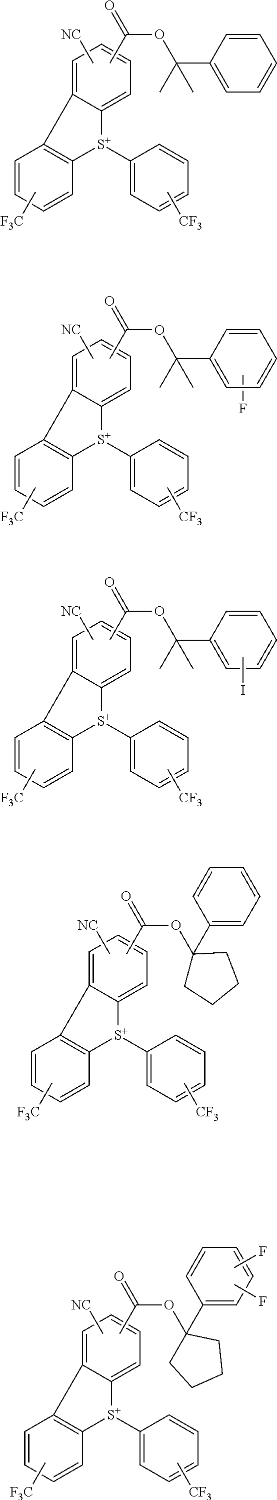

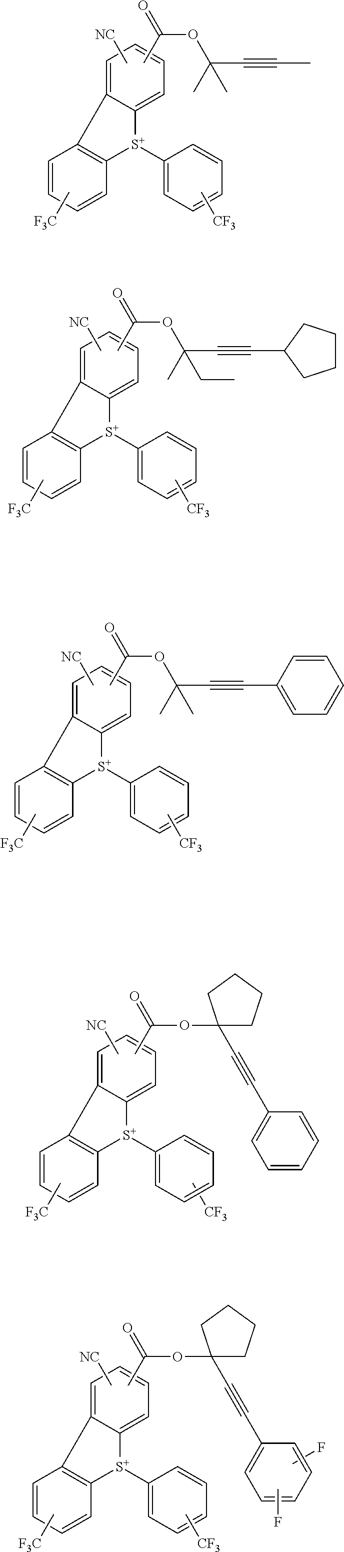

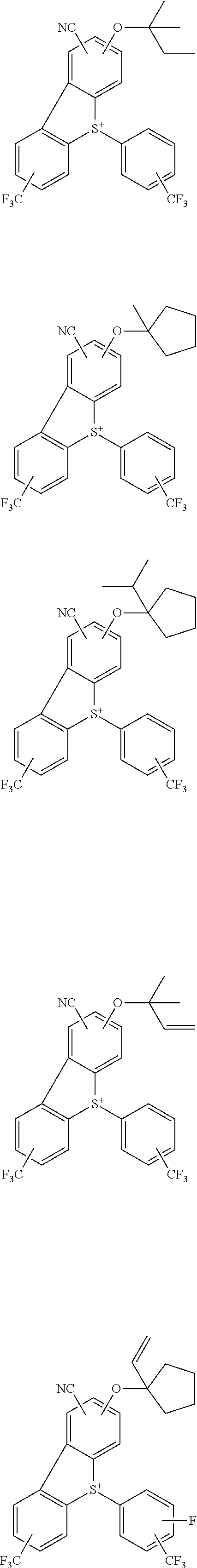

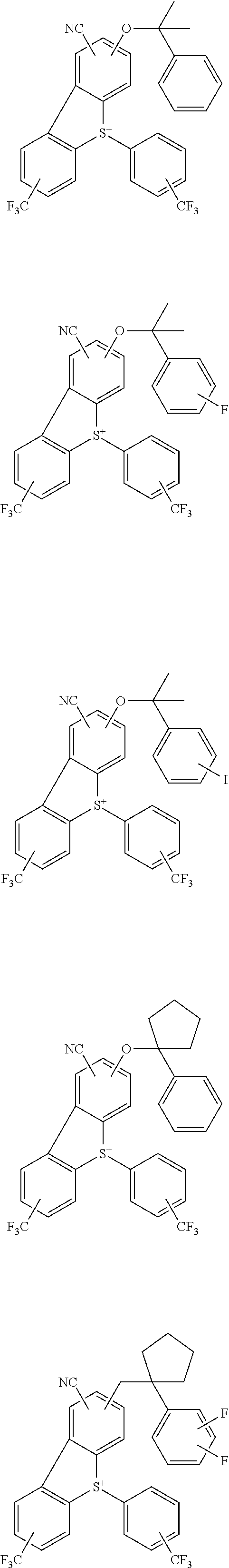

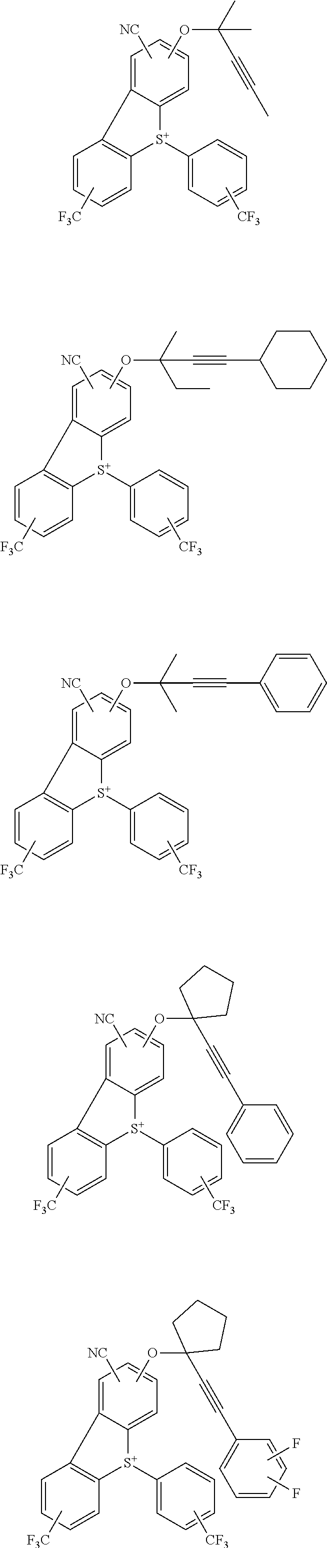

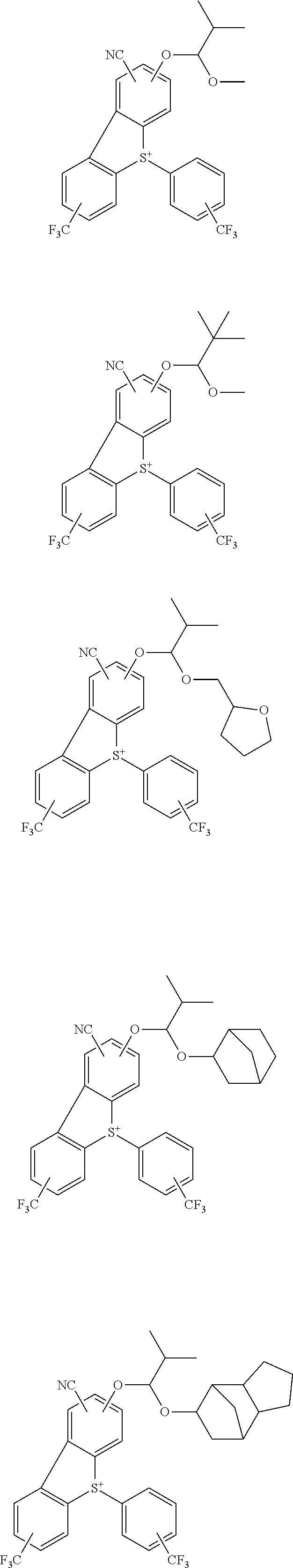

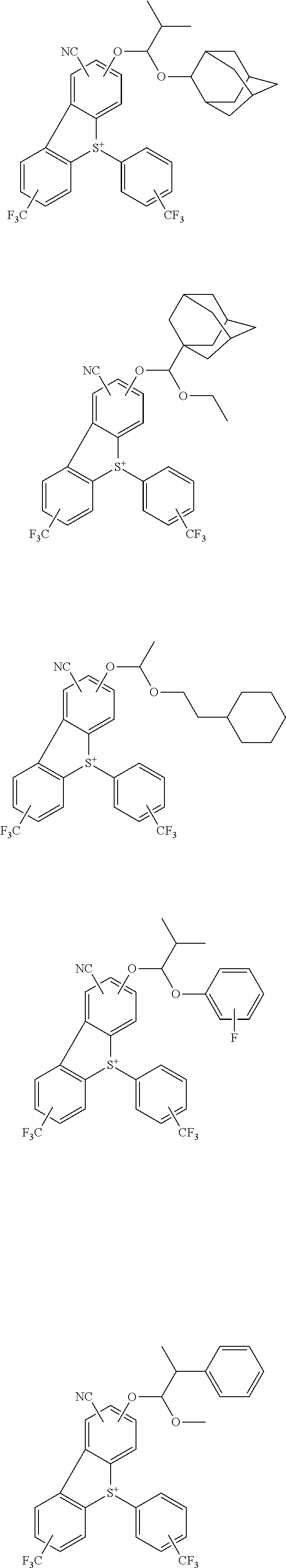

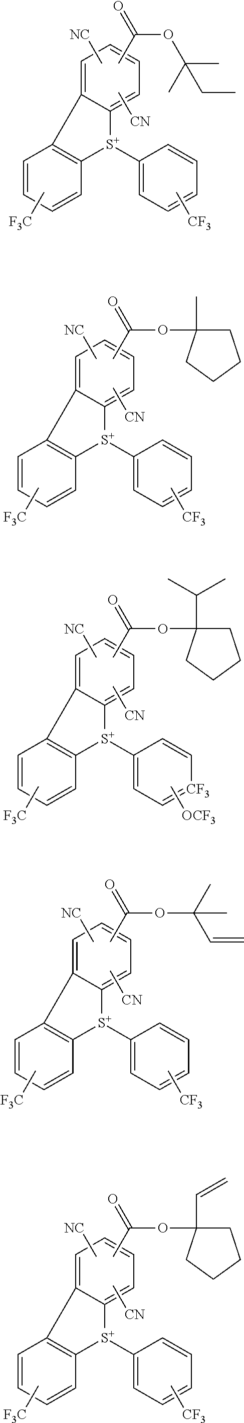

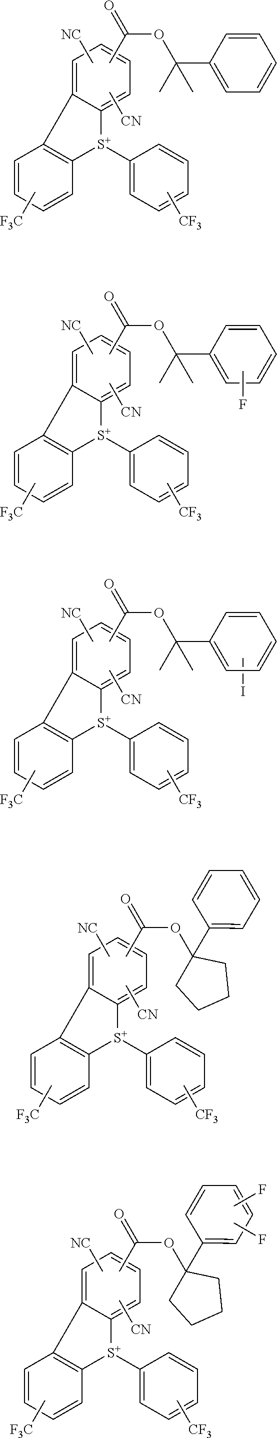

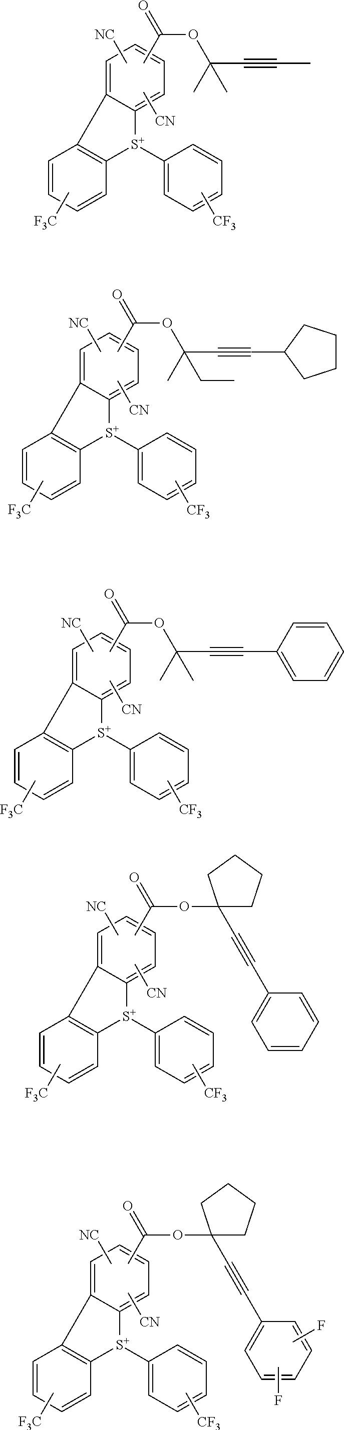

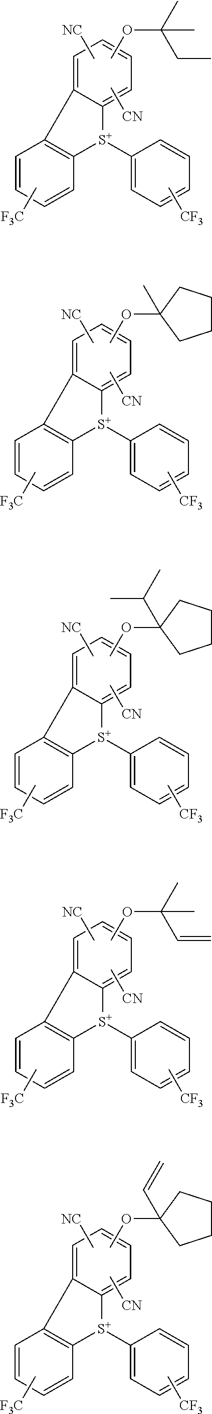

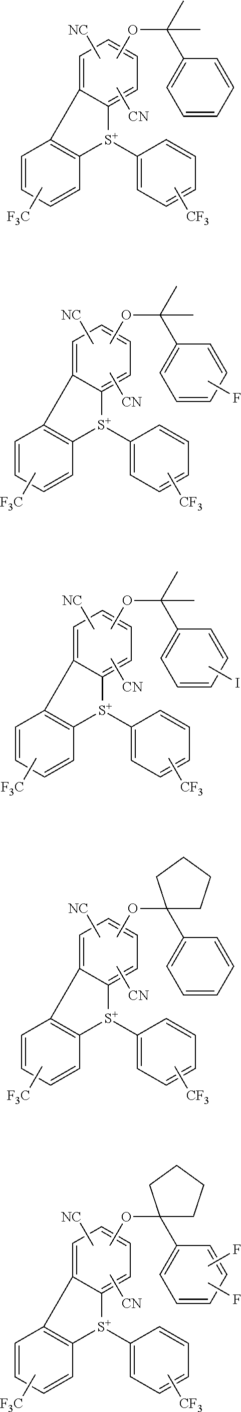

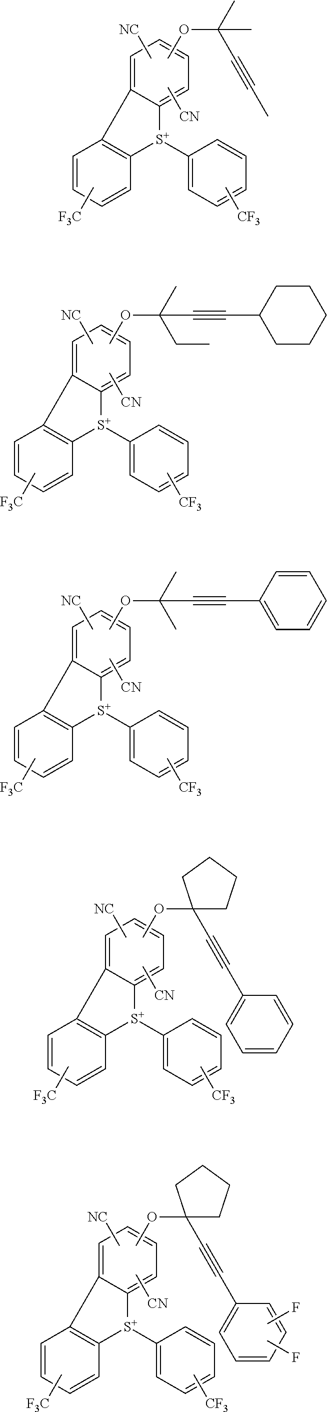

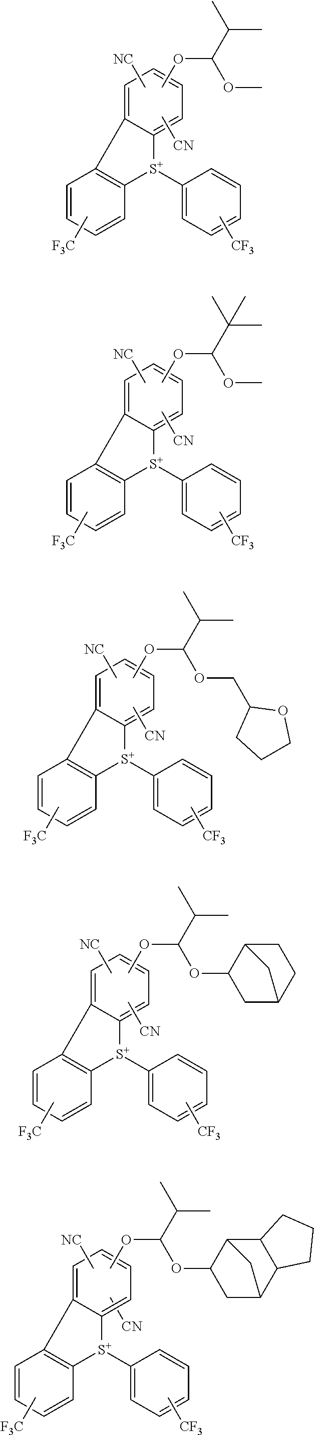

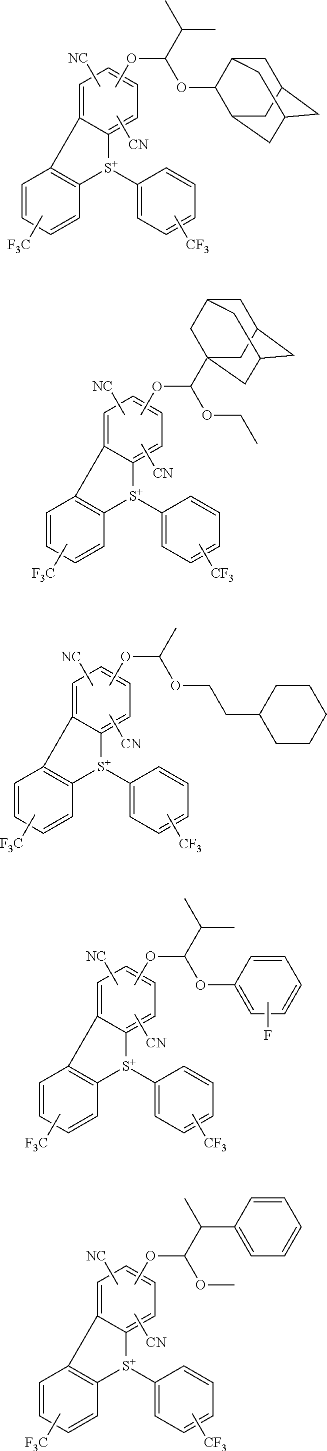

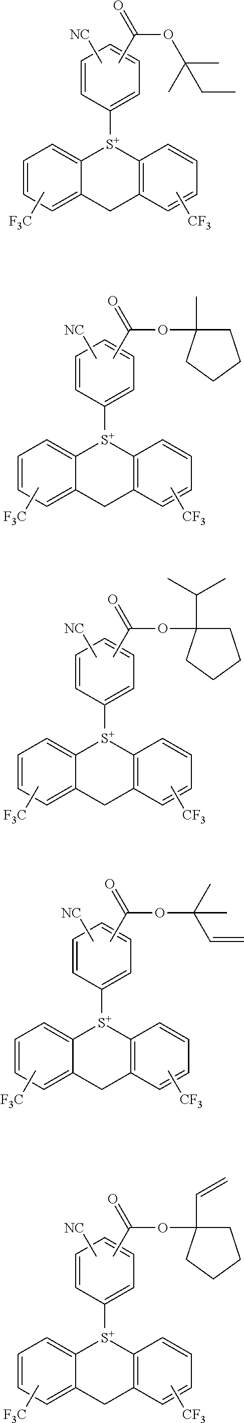

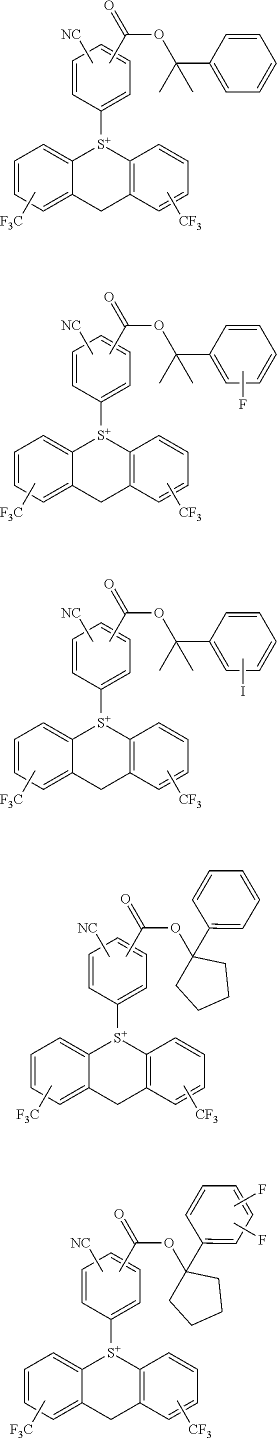

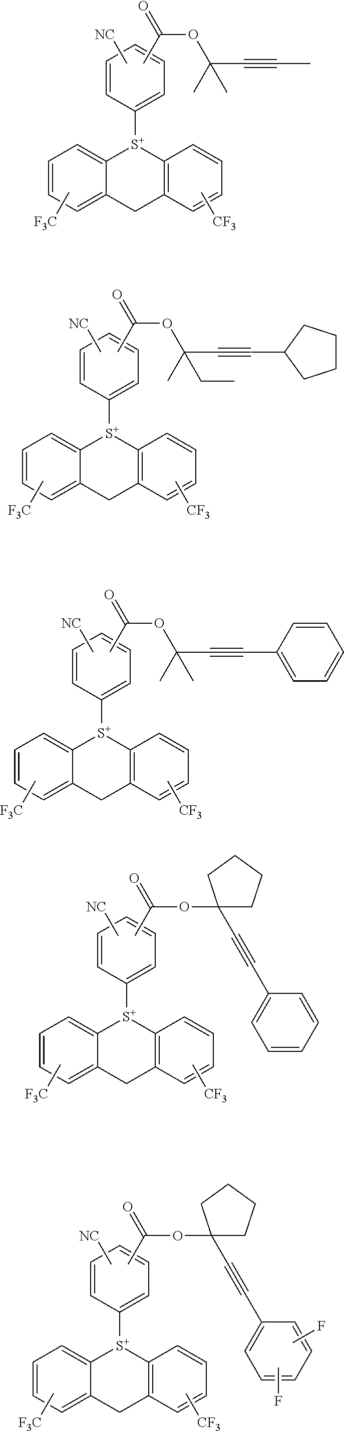

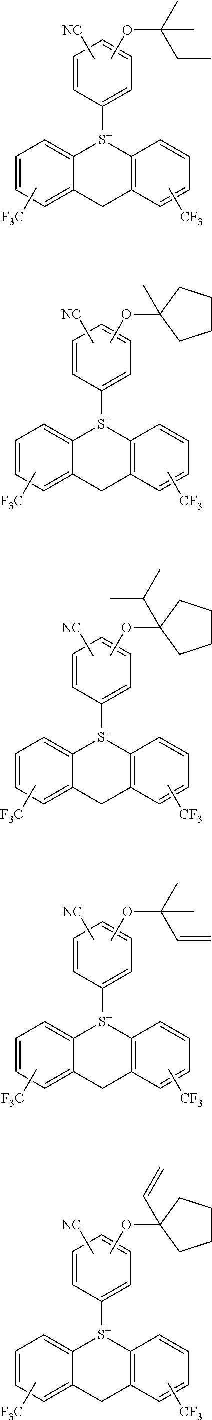

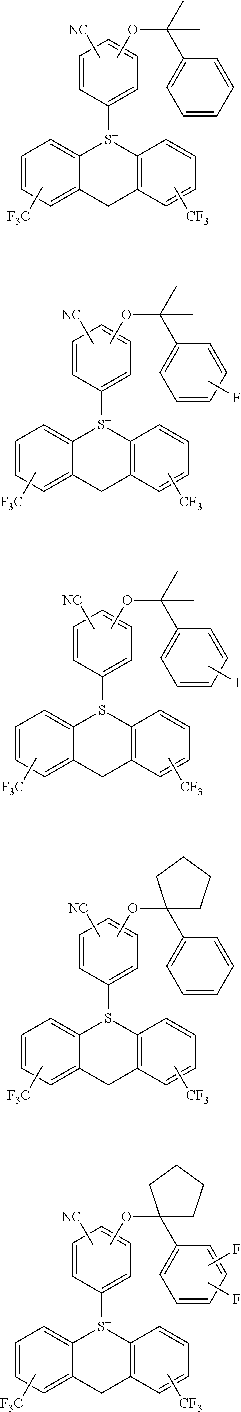

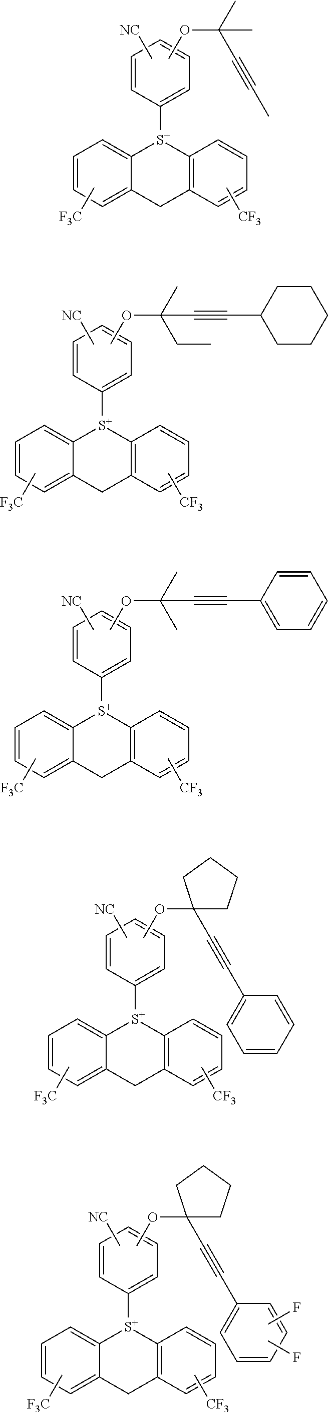

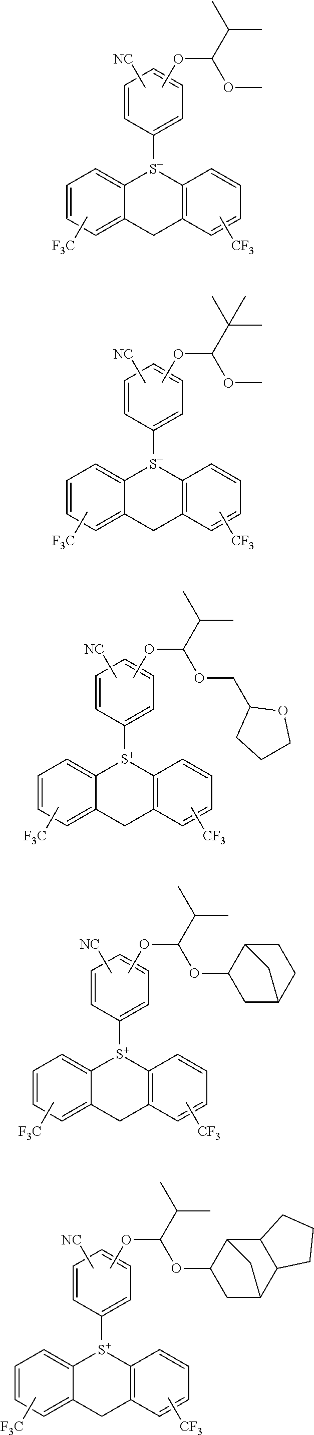

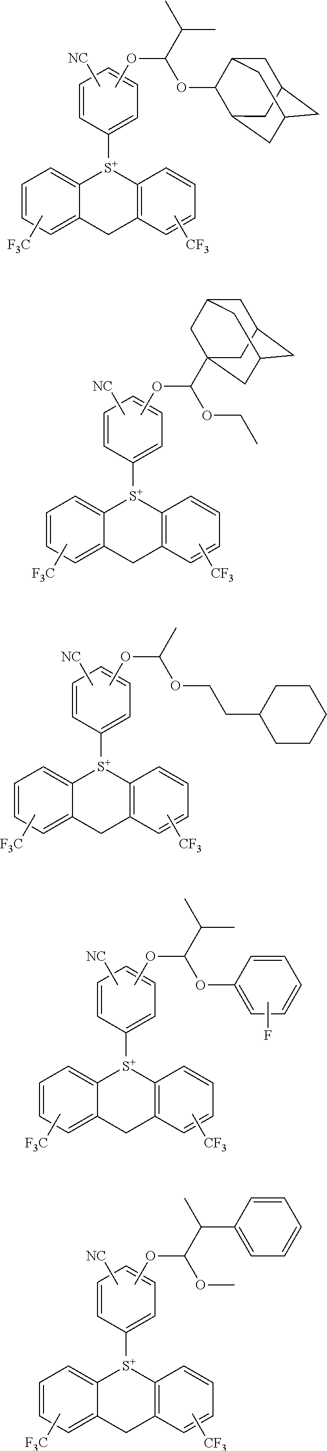

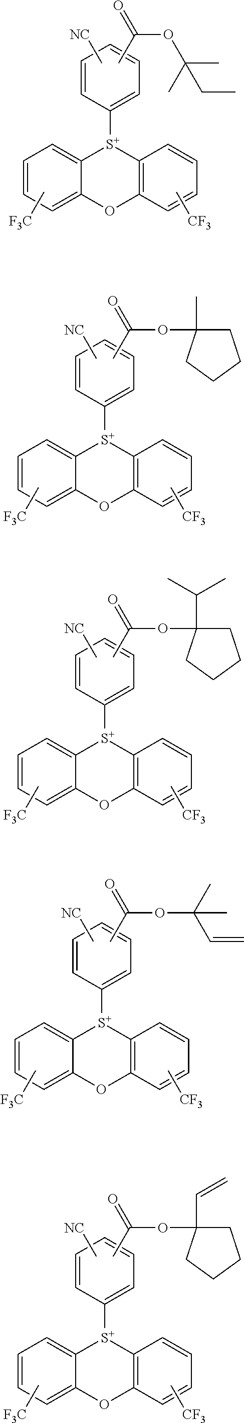

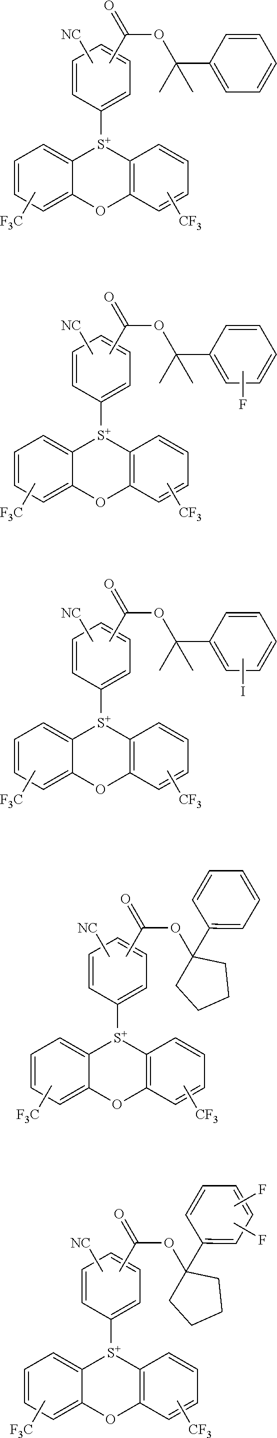

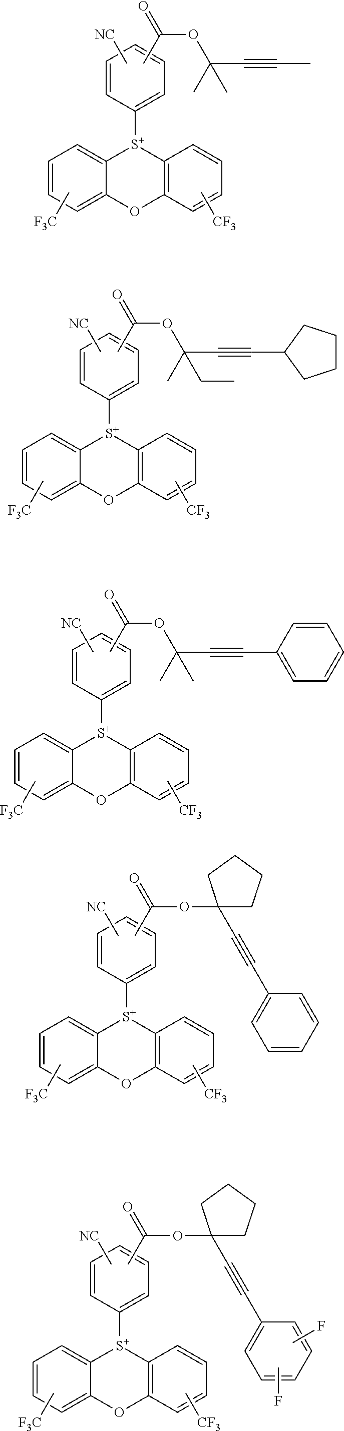

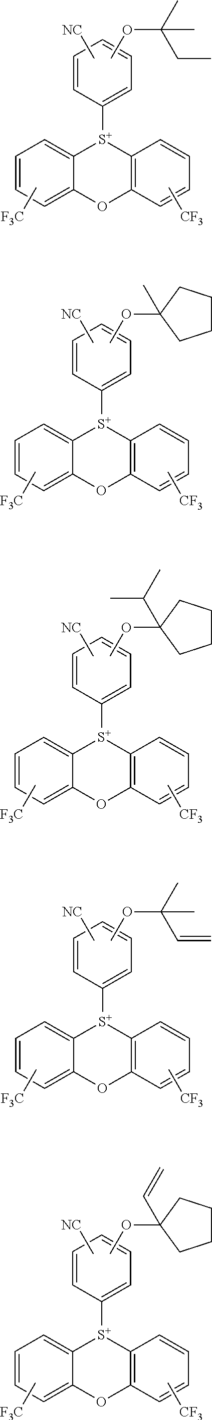

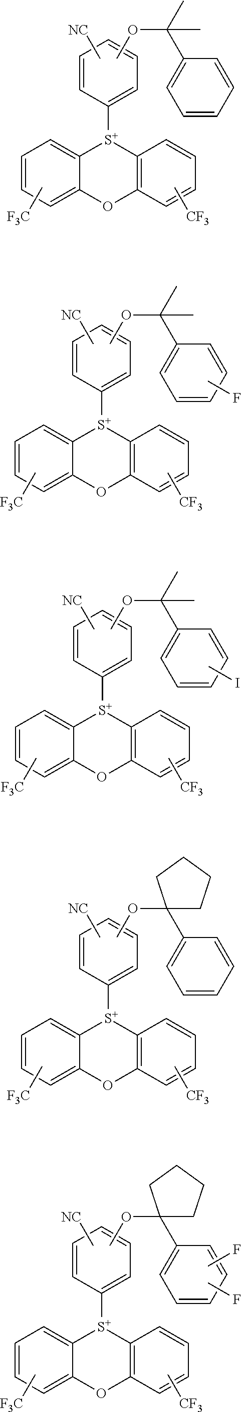

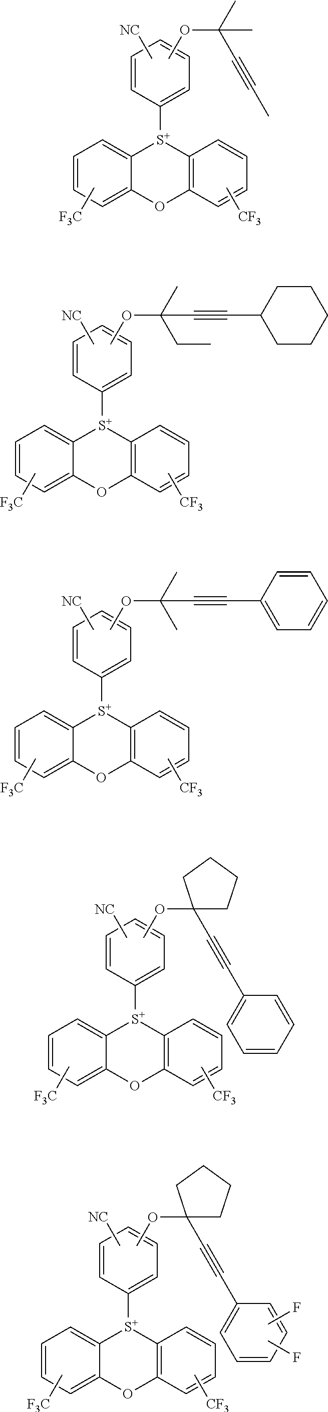

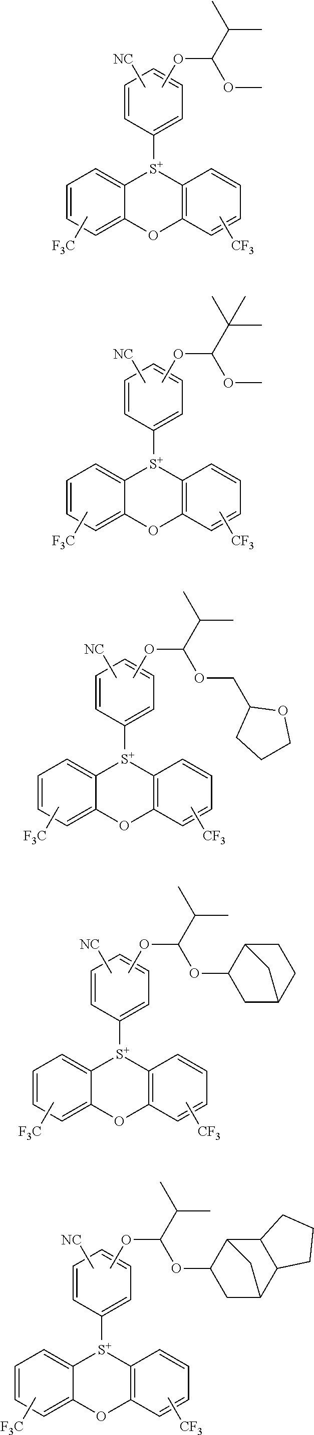

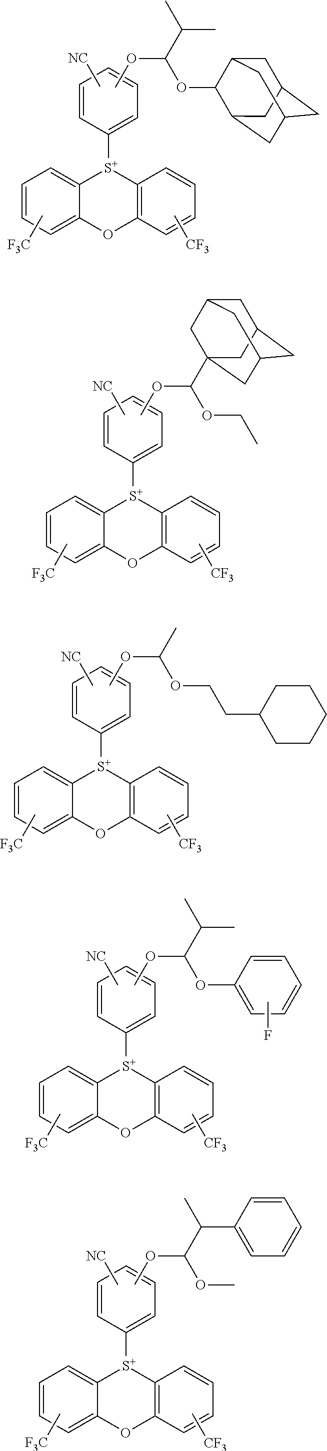

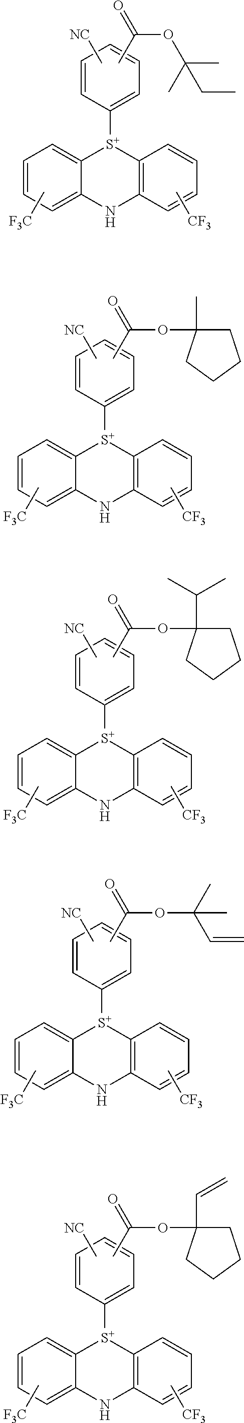

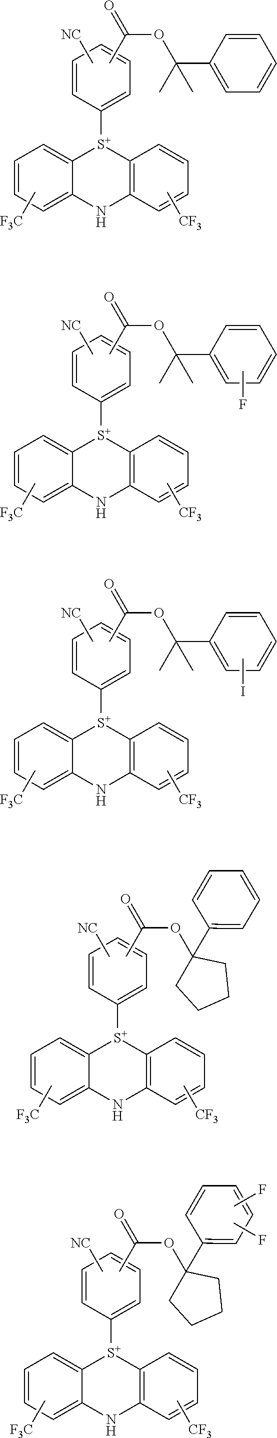

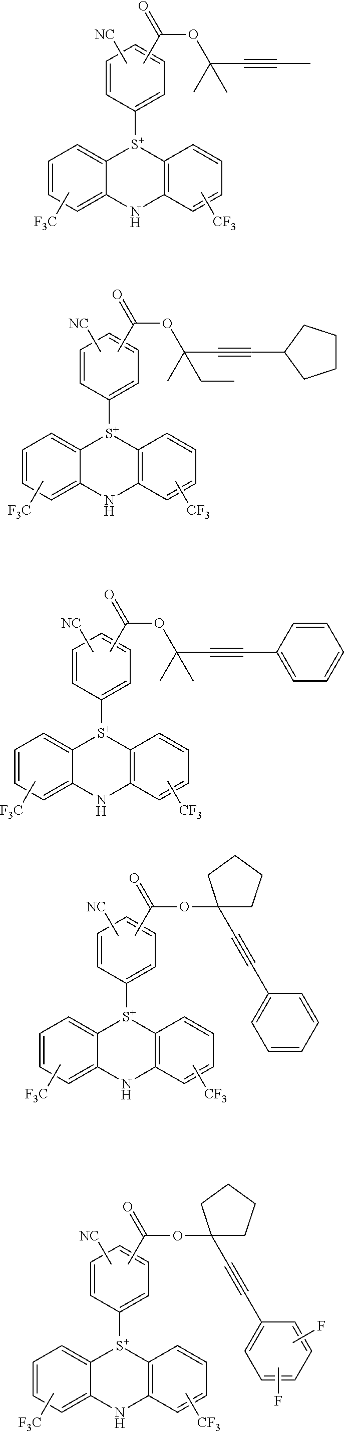

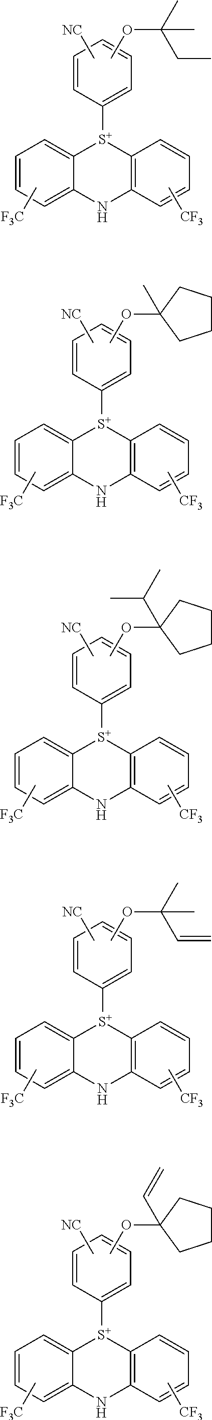

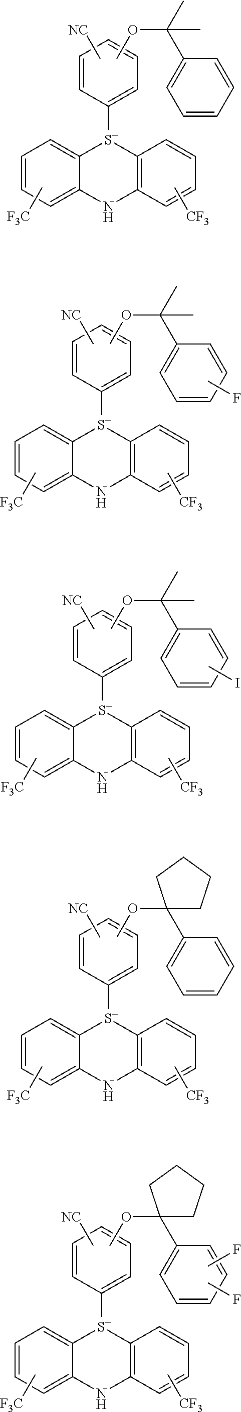

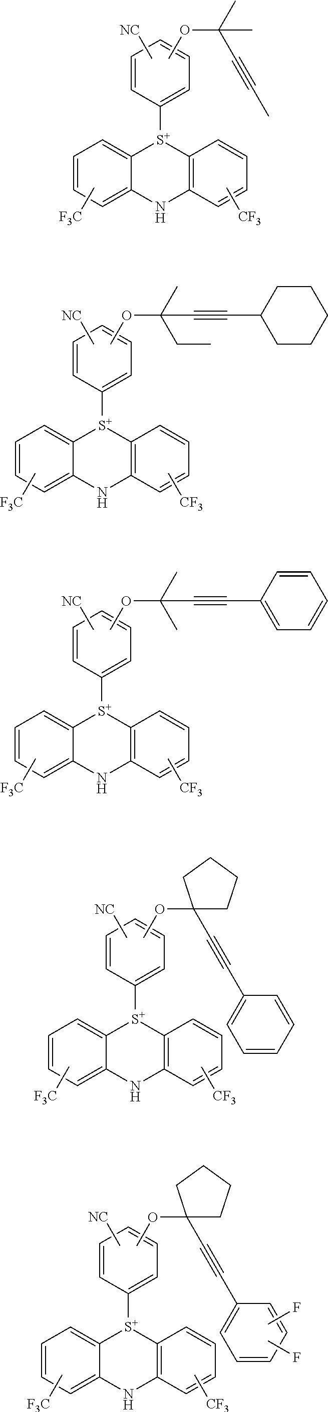

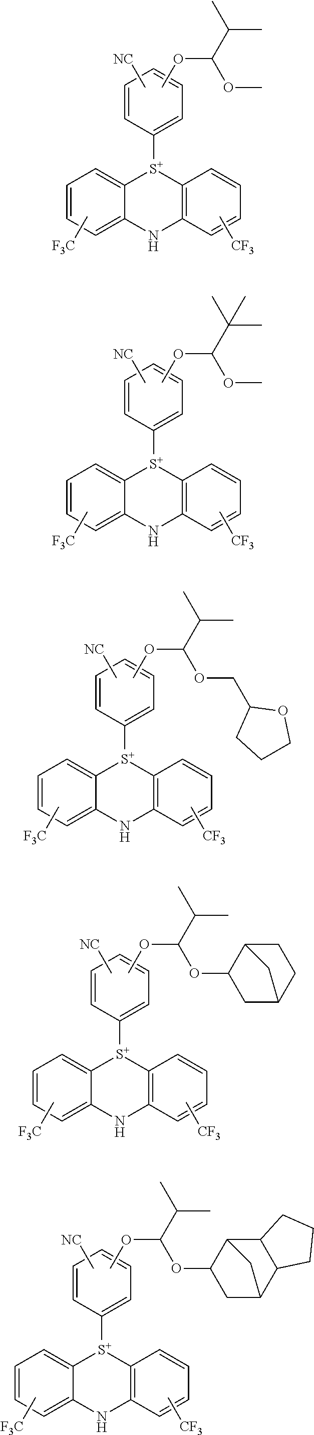

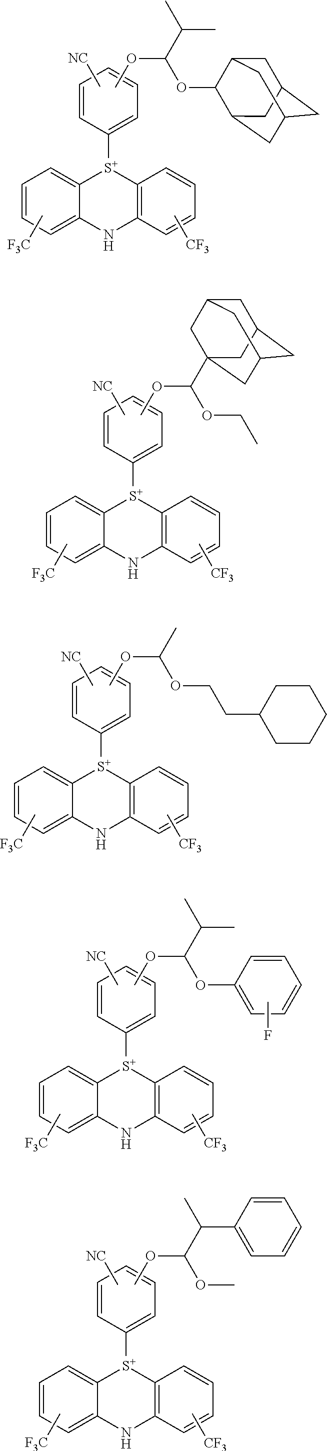

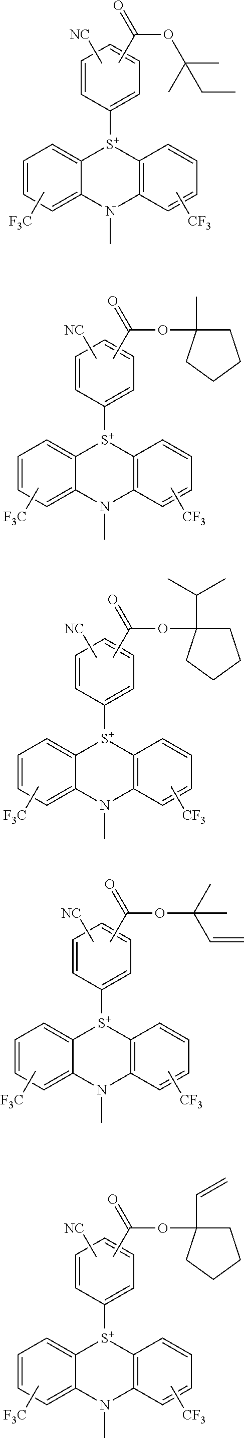

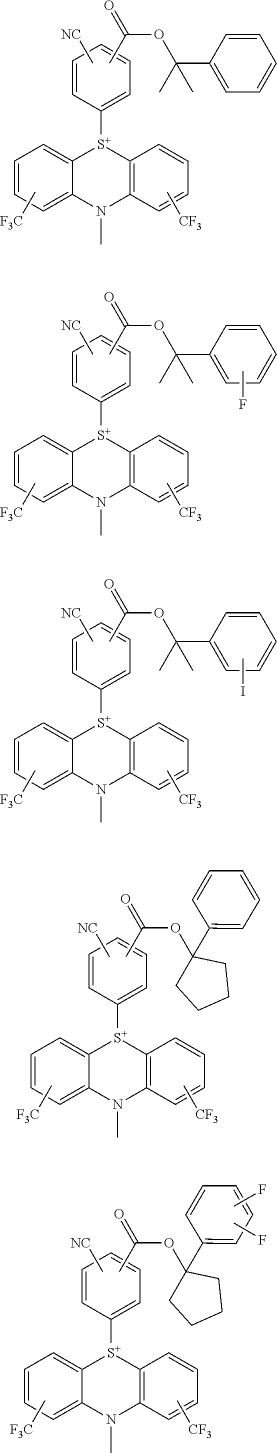

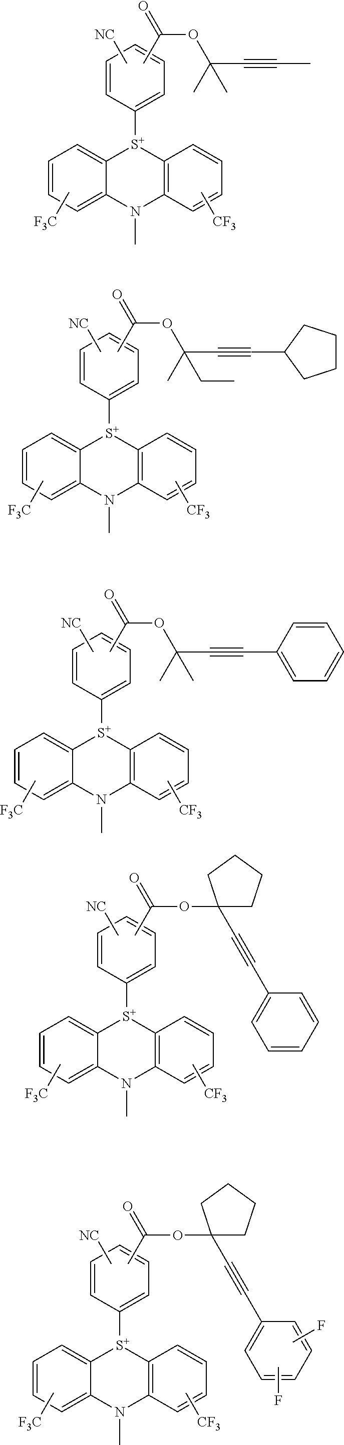

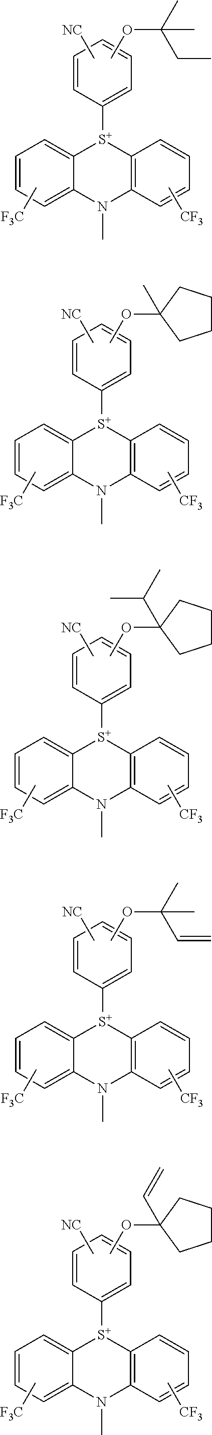

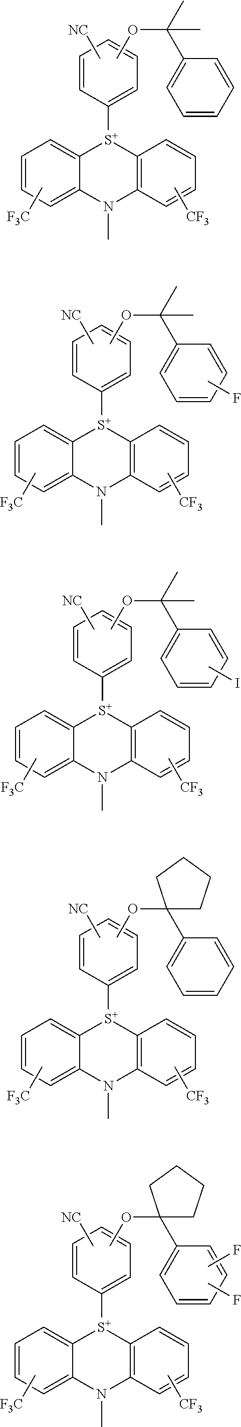

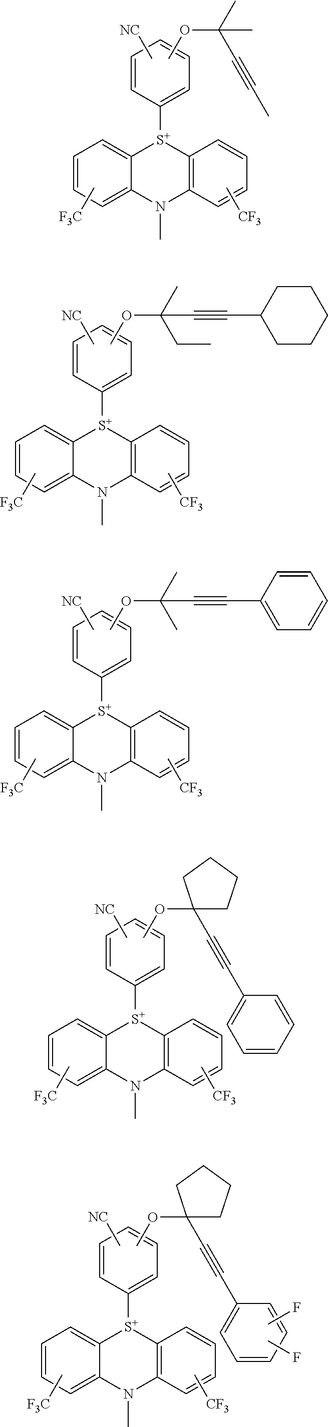

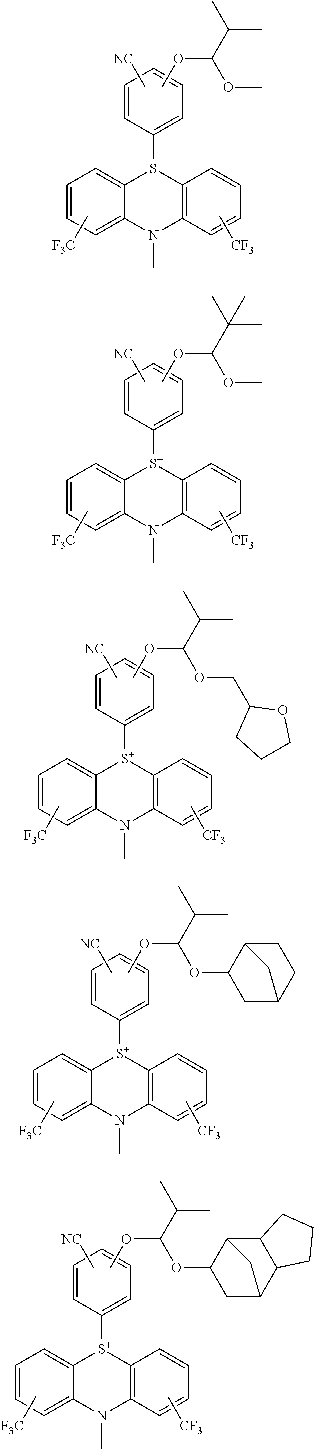

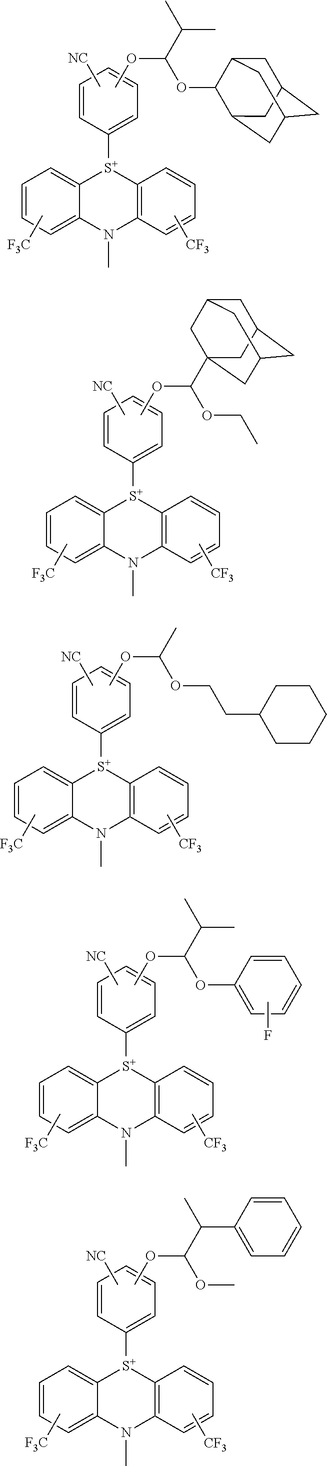

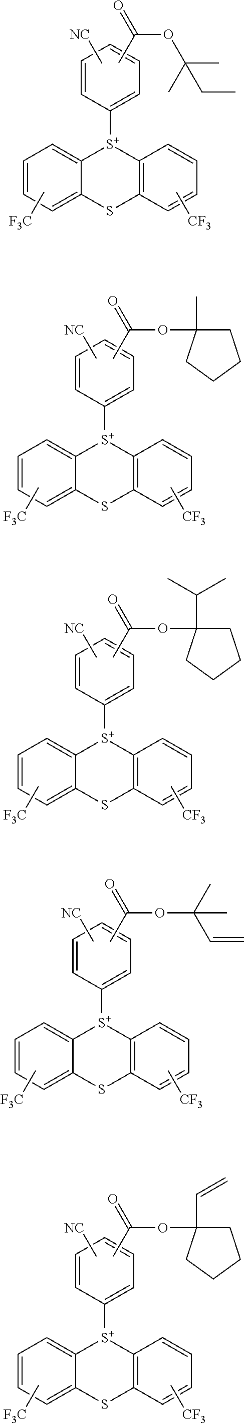

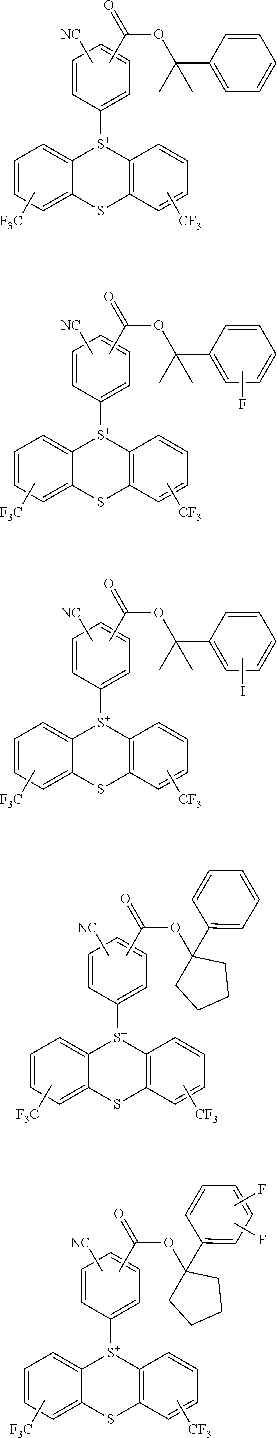

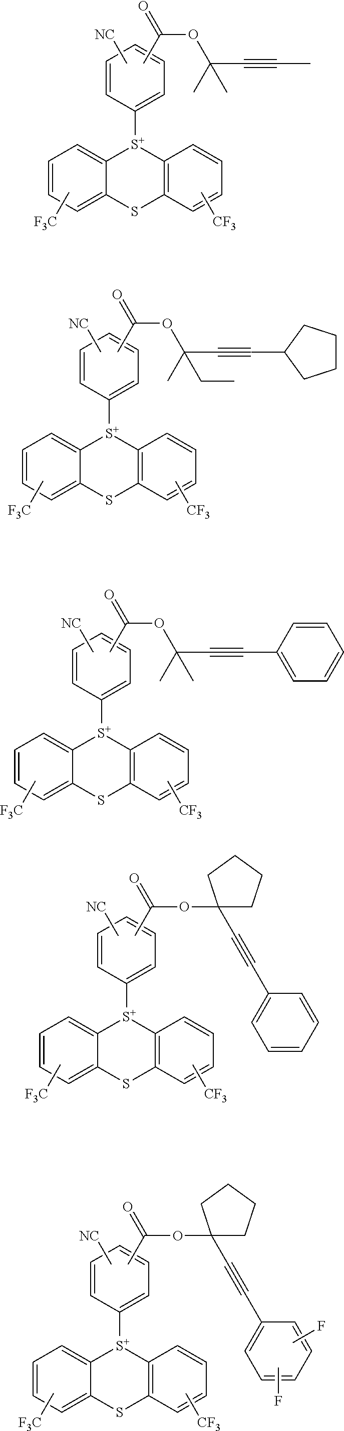

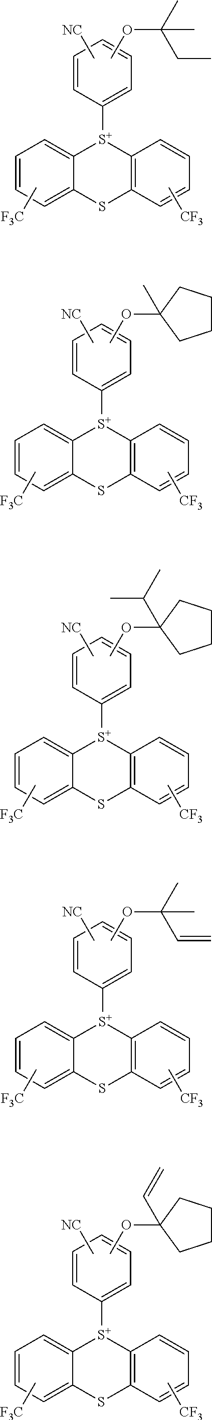

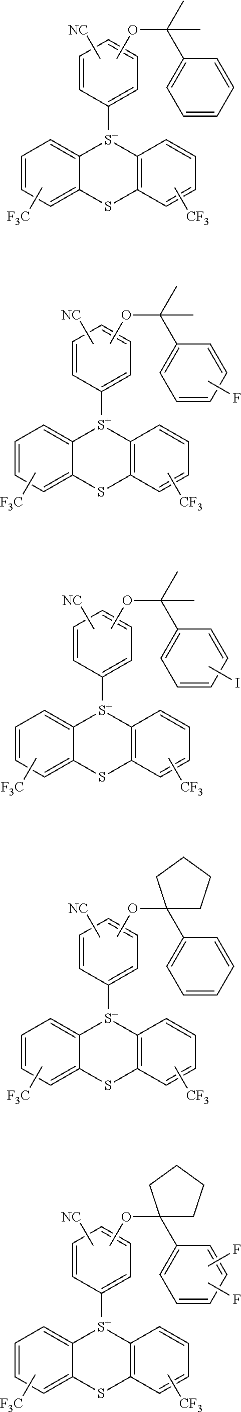

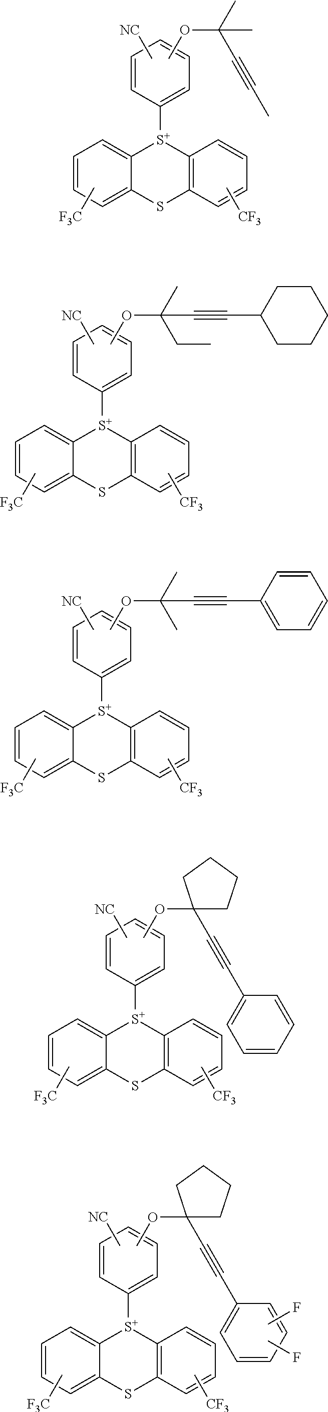

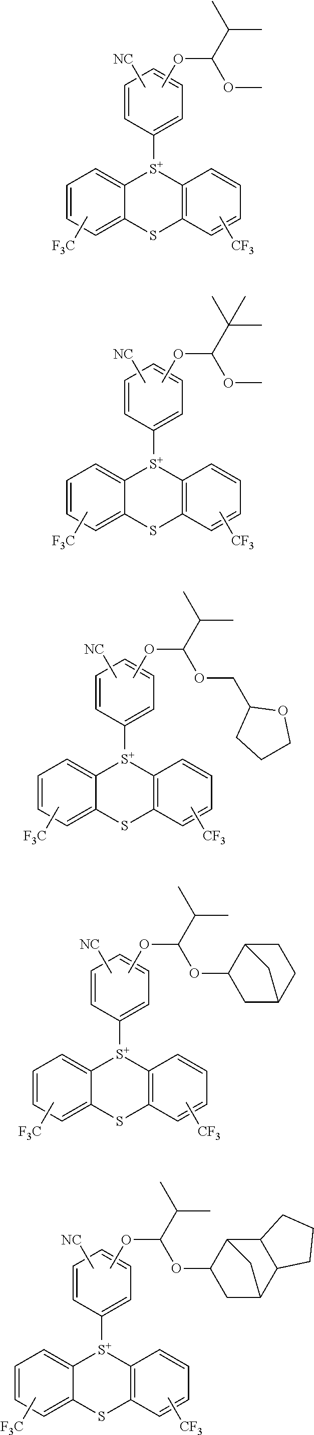

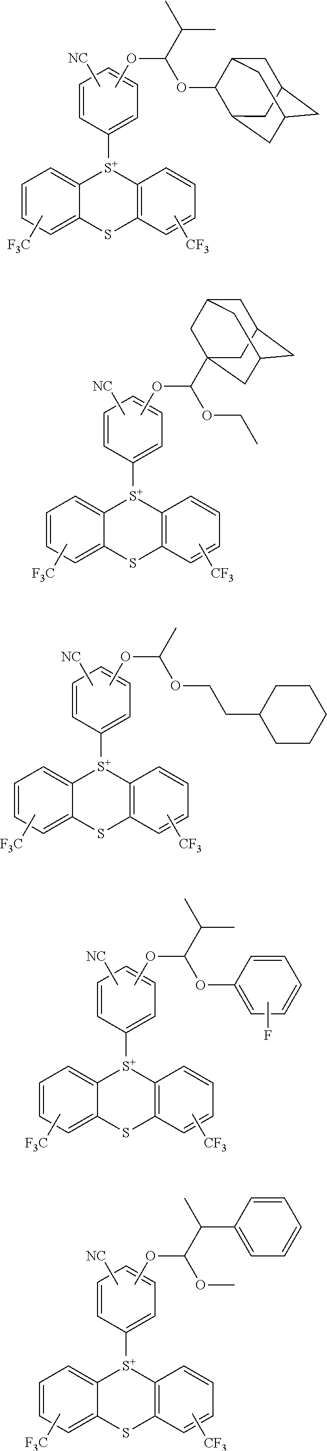

[chemical structure]

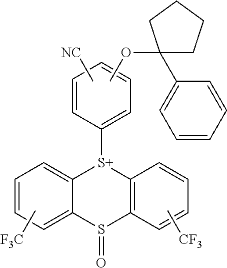

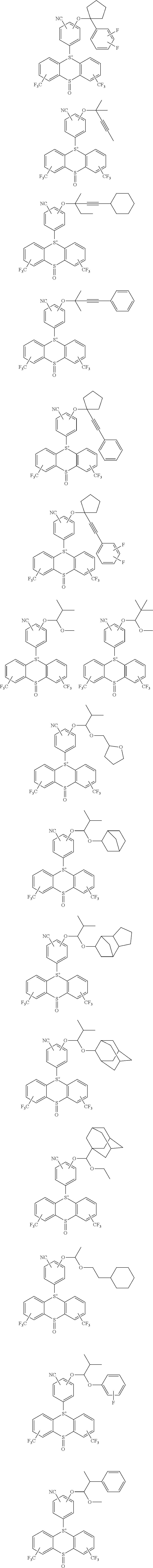

[chemical structure]

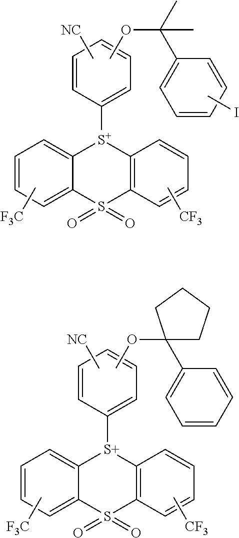

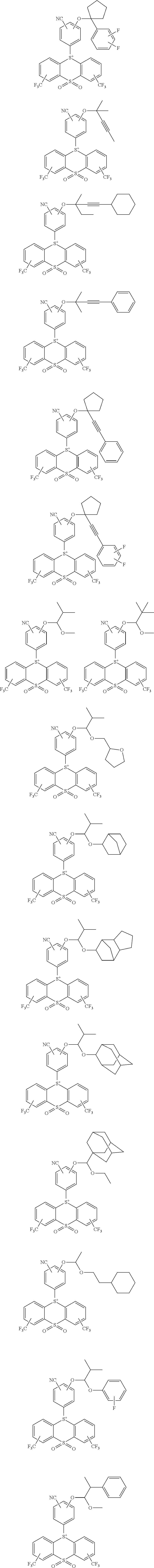

[chemical structure]

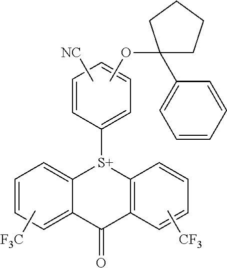

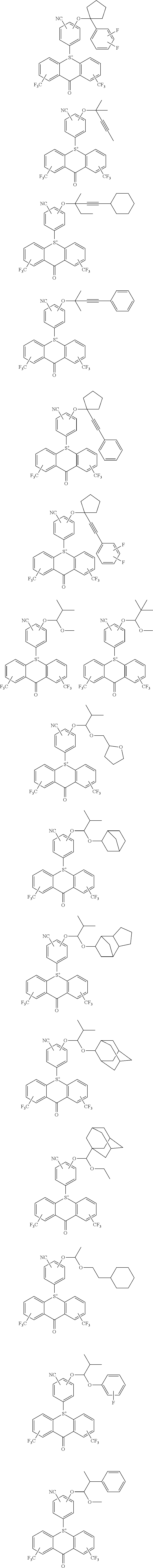

[chemical structure]

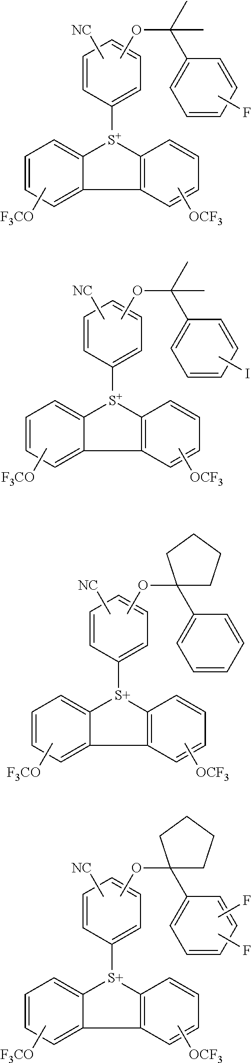

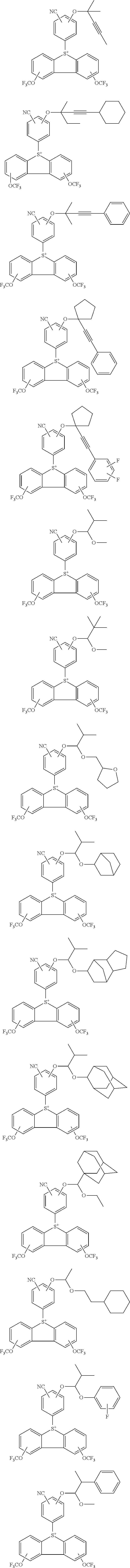

[chemical structure]

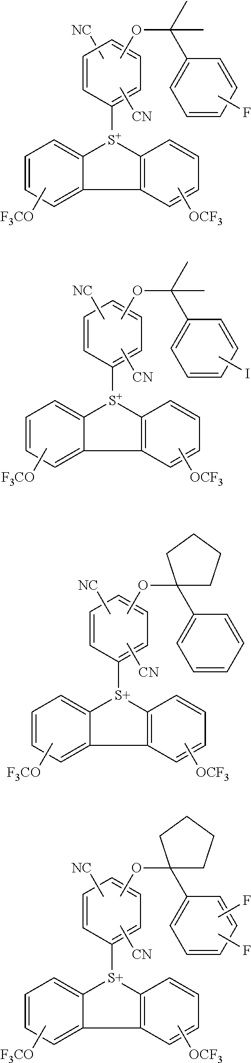

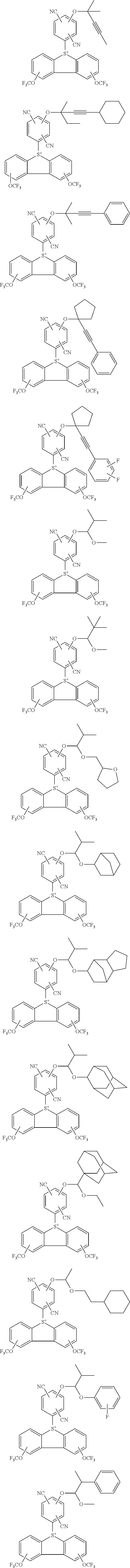

[chemical structure]

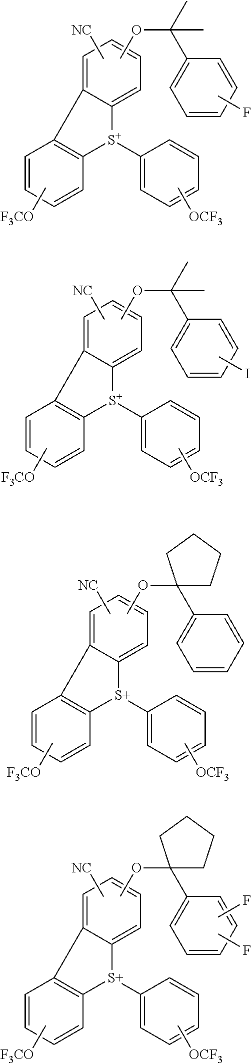

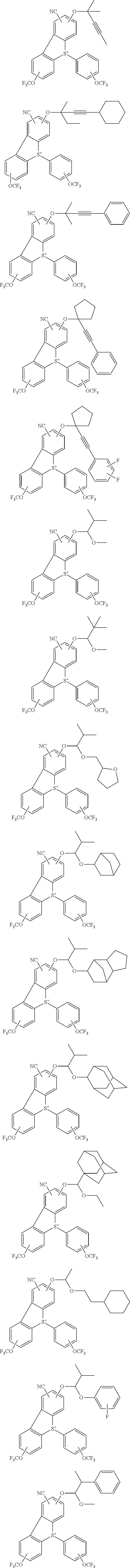

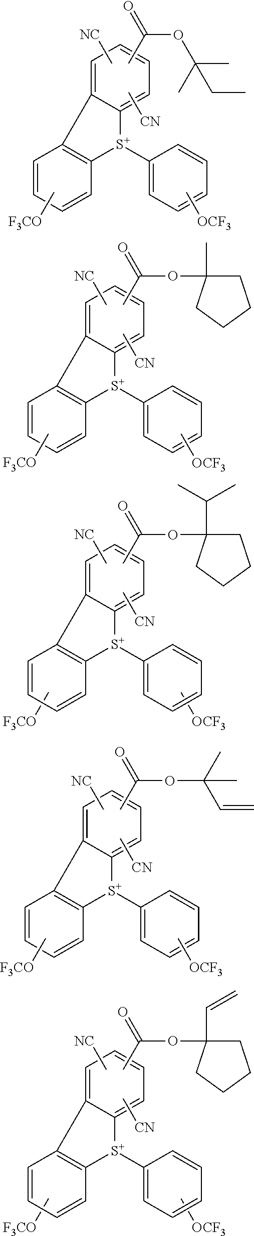

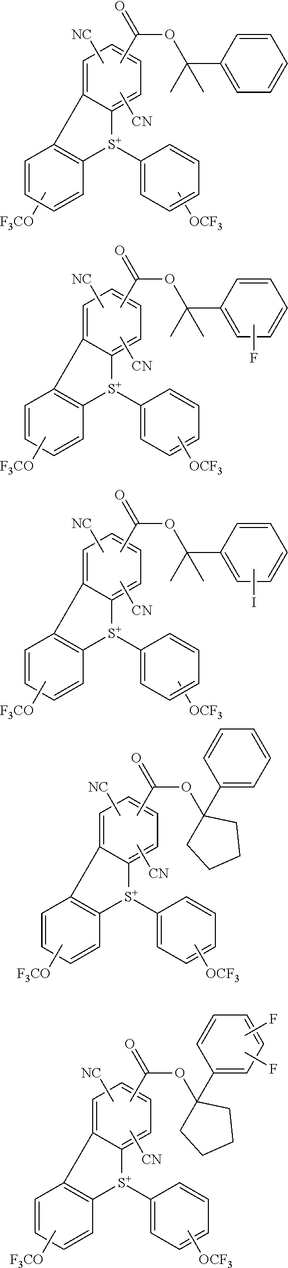

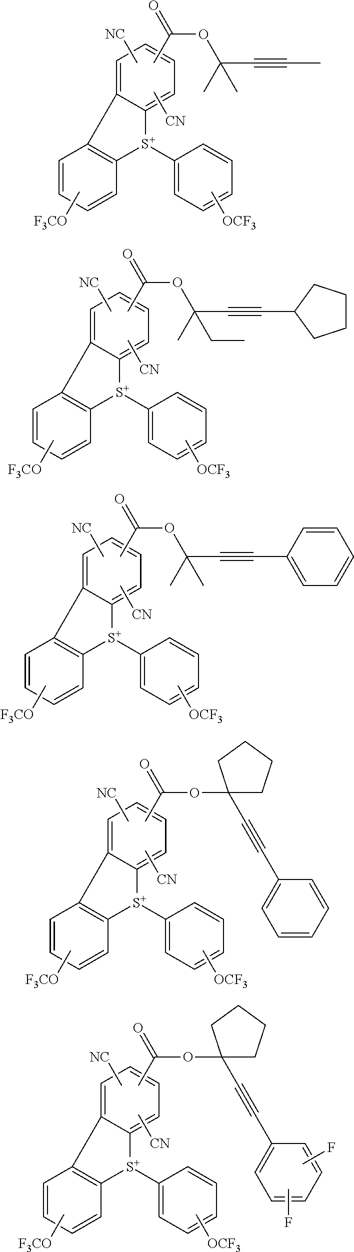

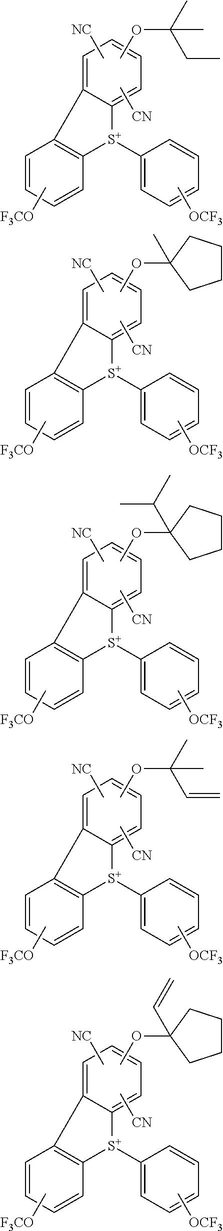

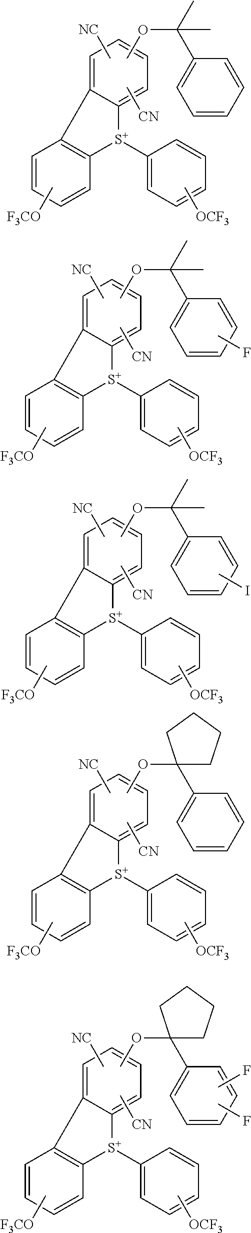

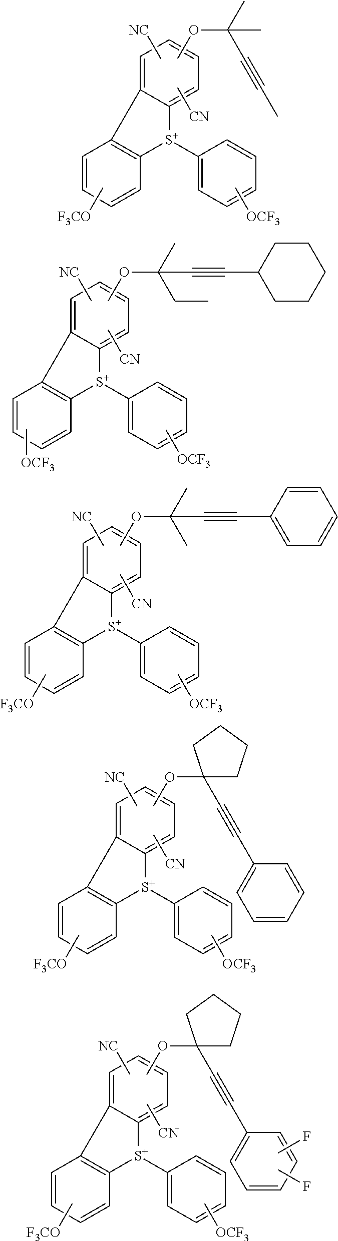

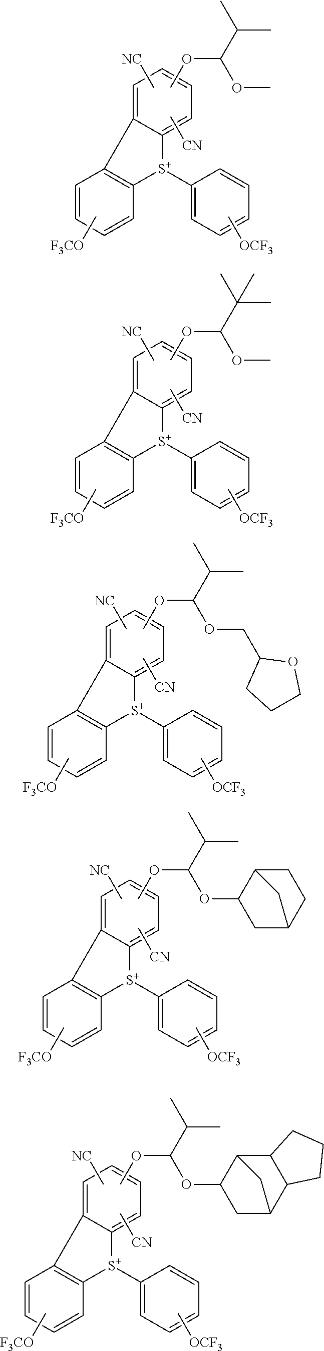

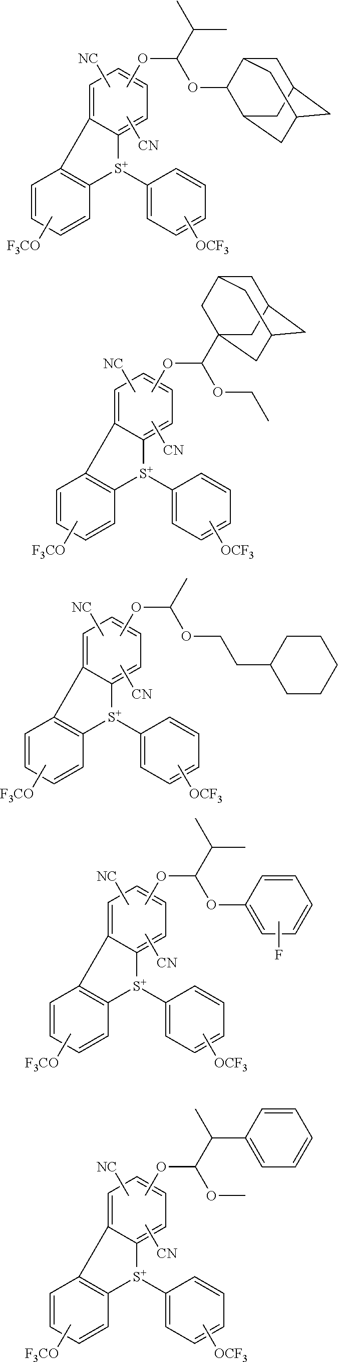

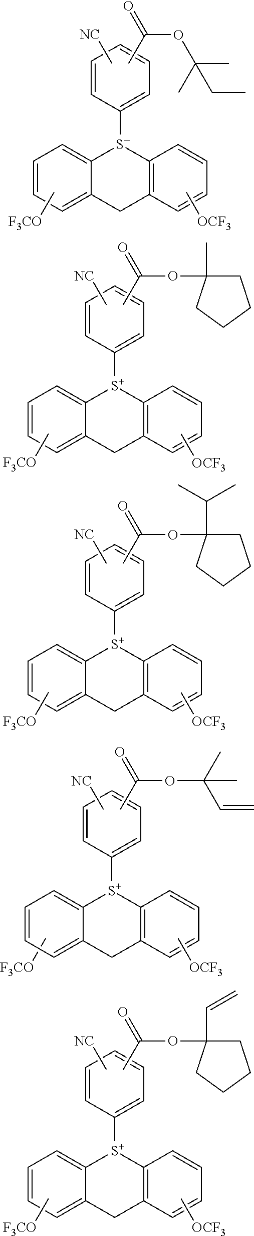

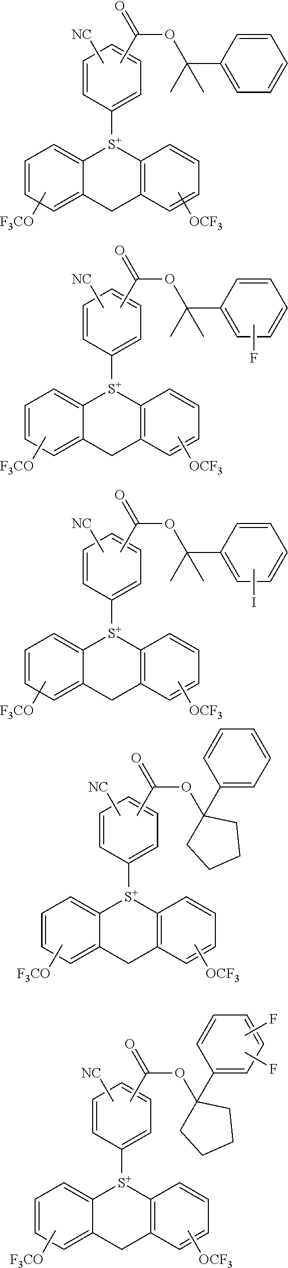

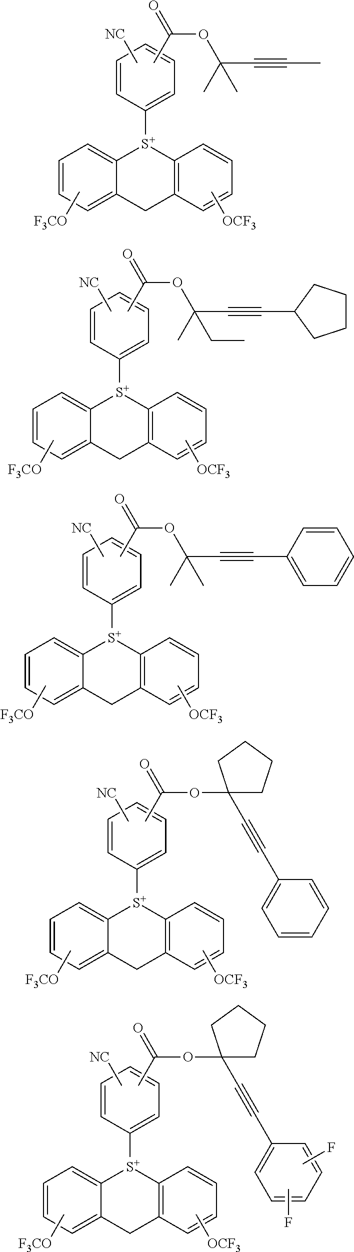

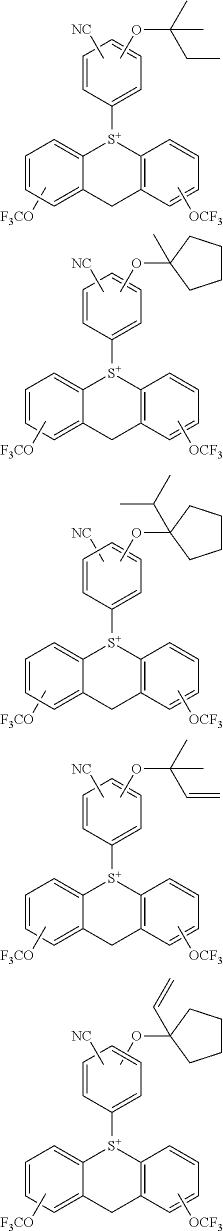

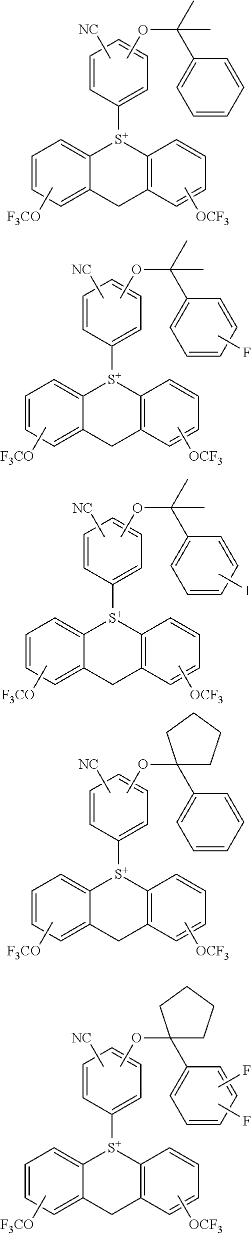

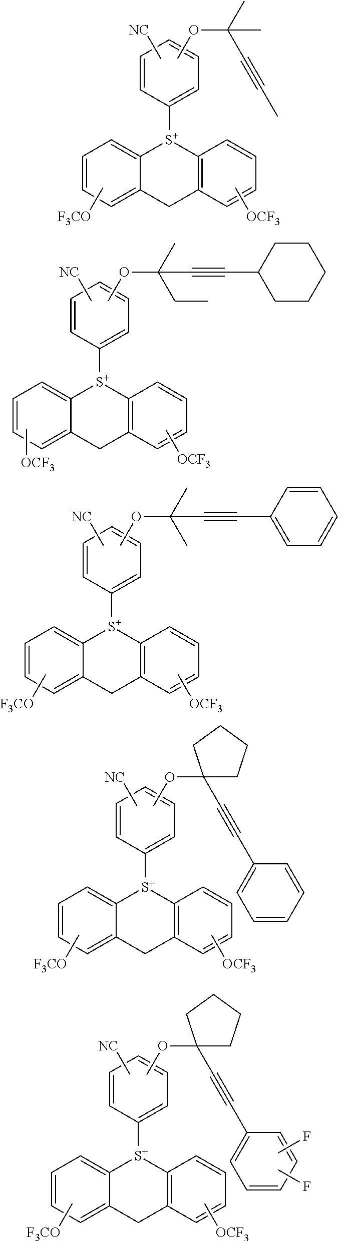

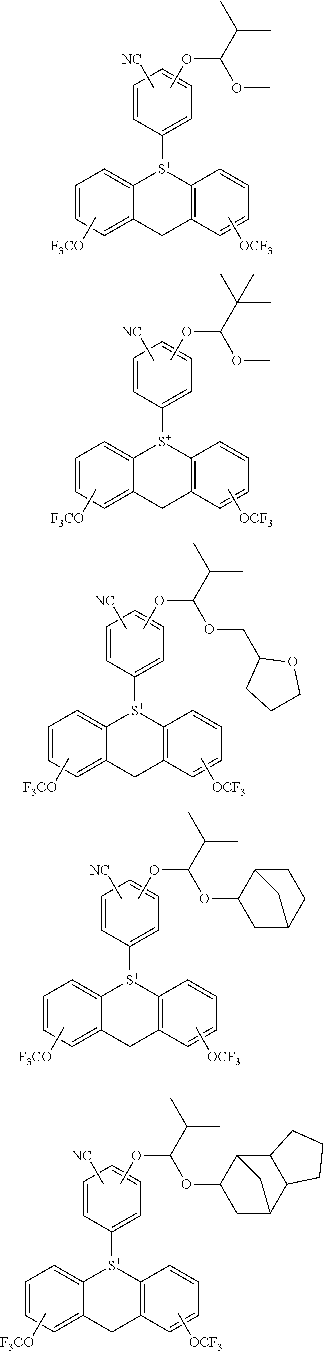

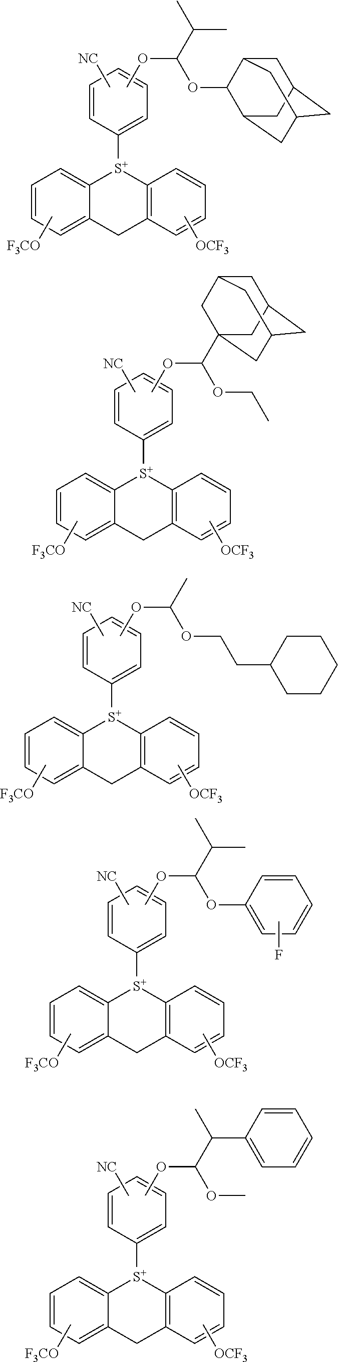

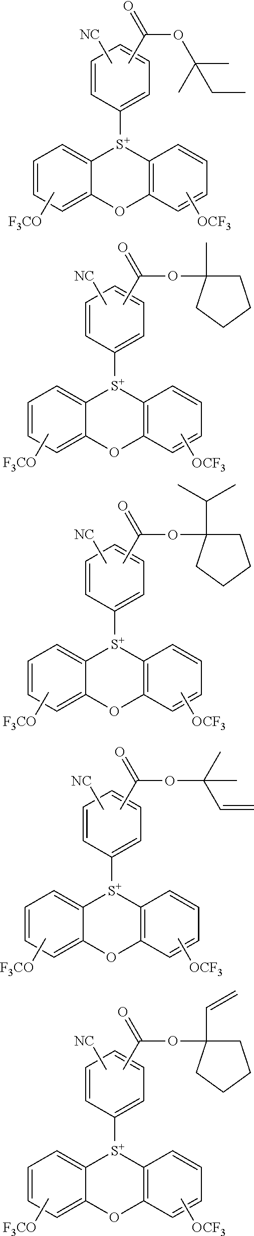

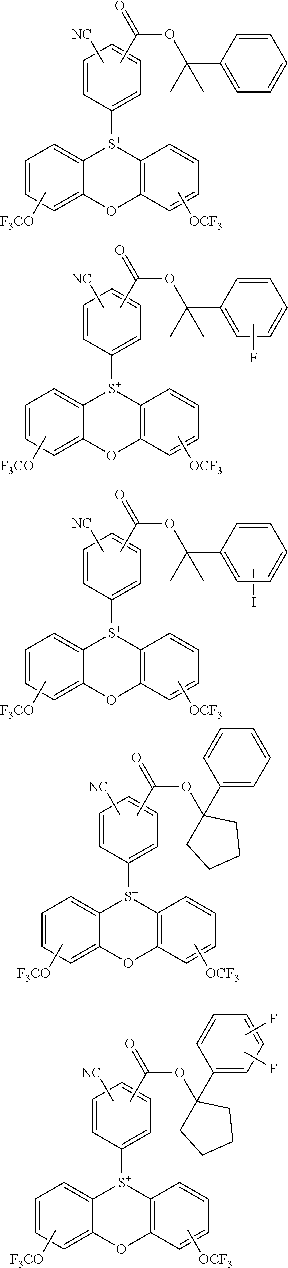

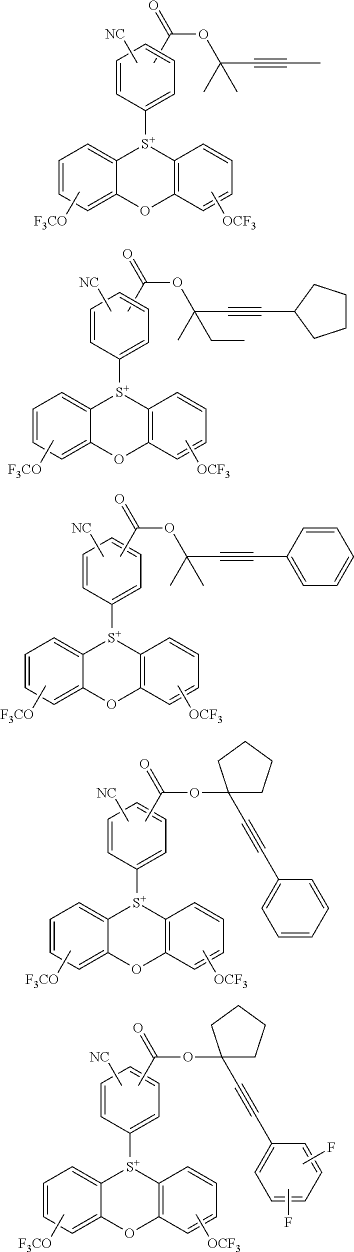

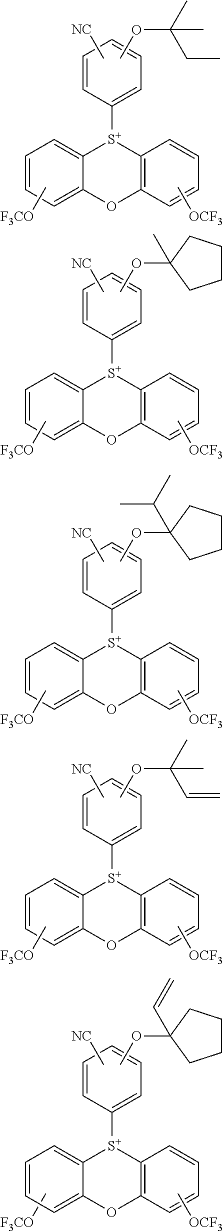

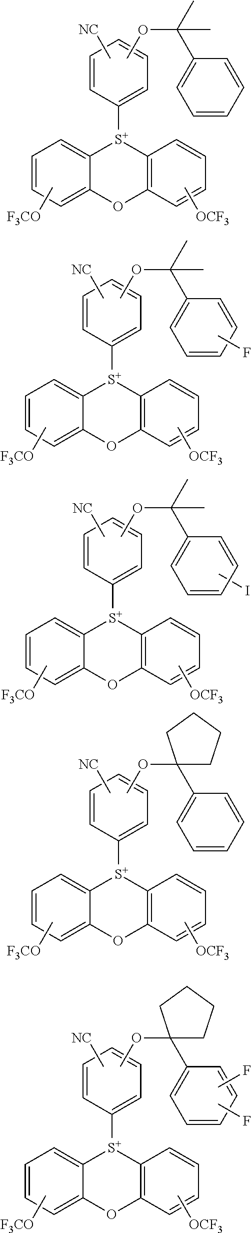

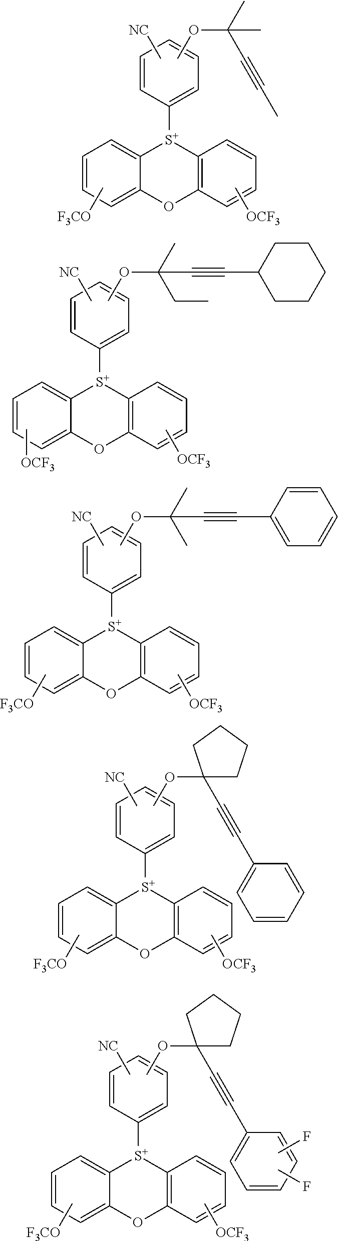

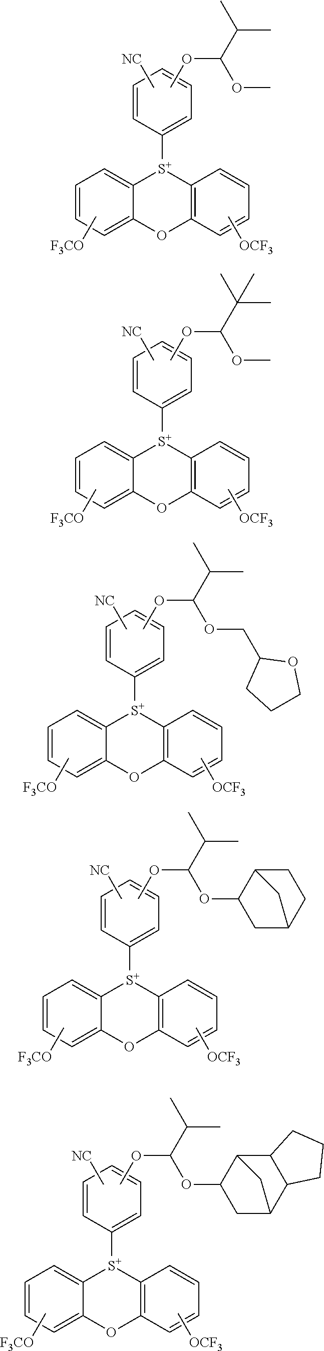

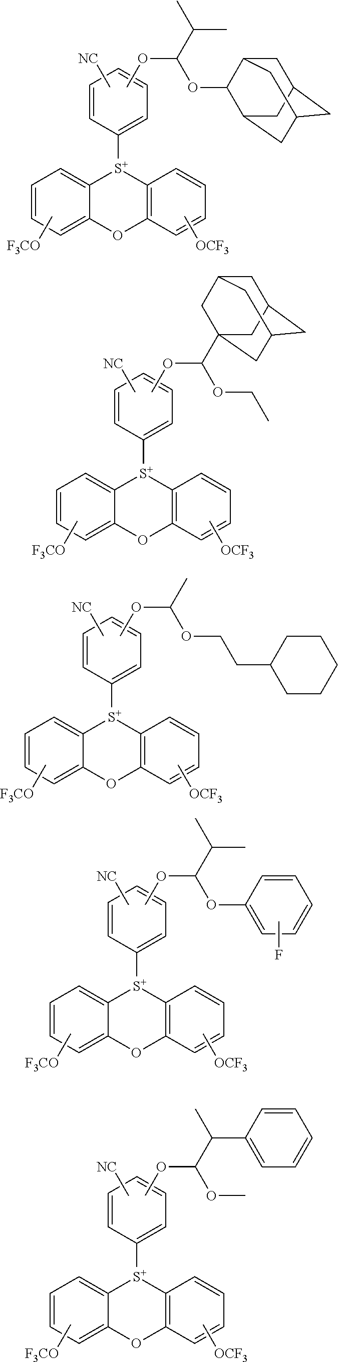

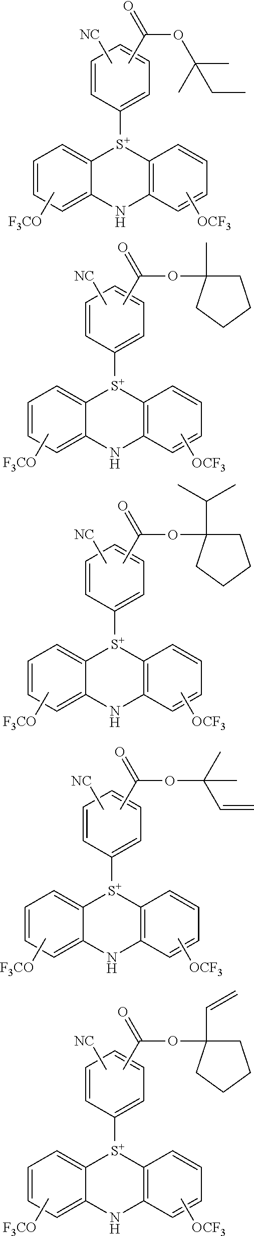

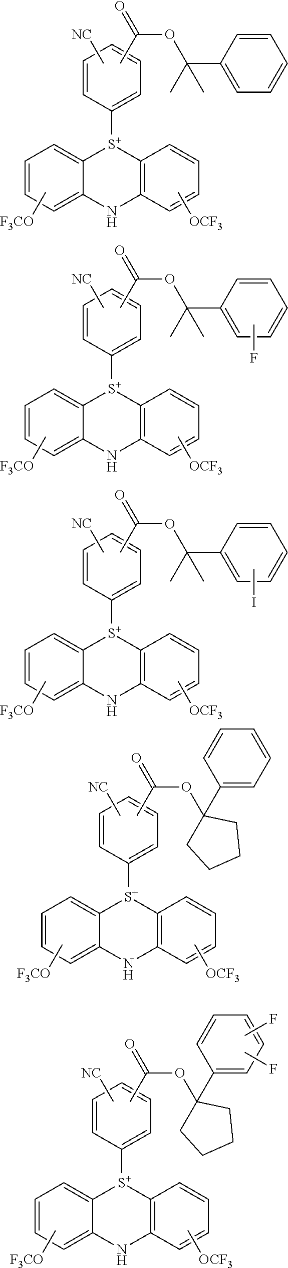

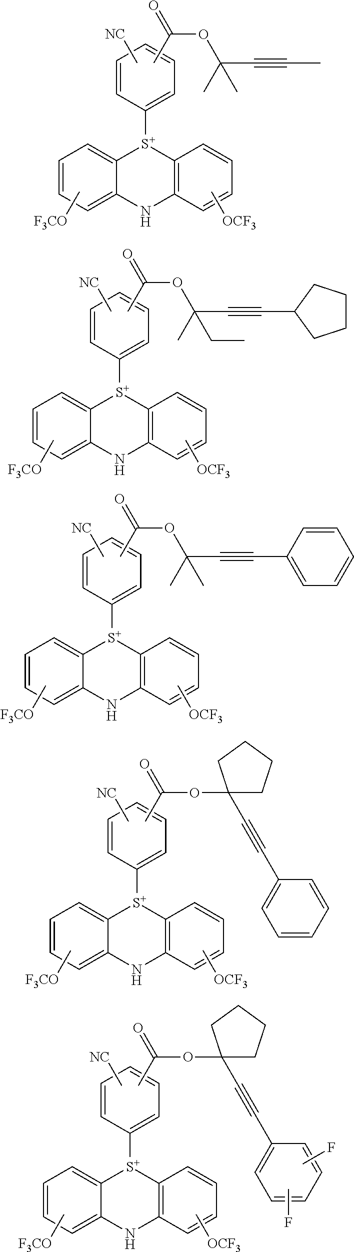

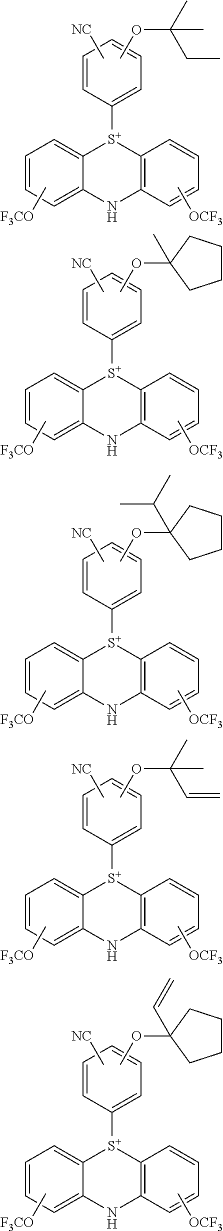

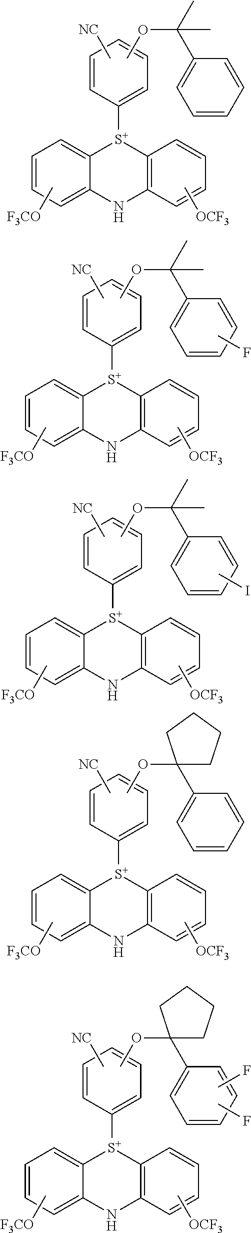

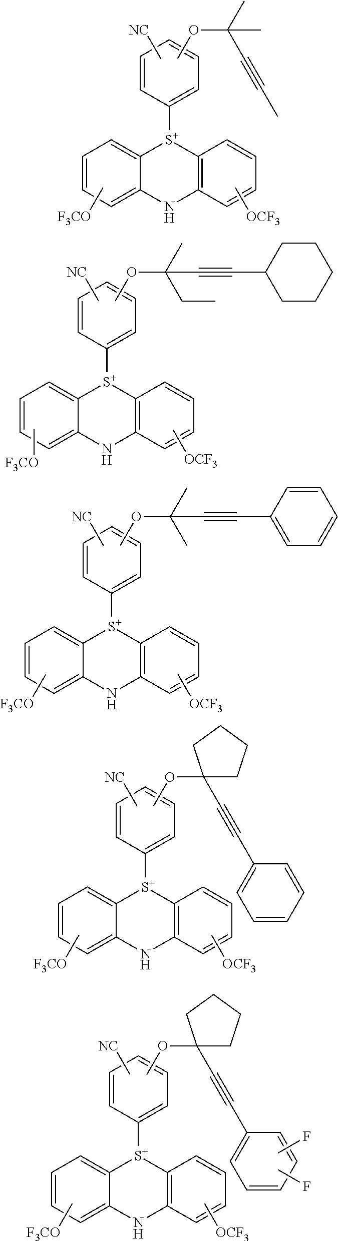

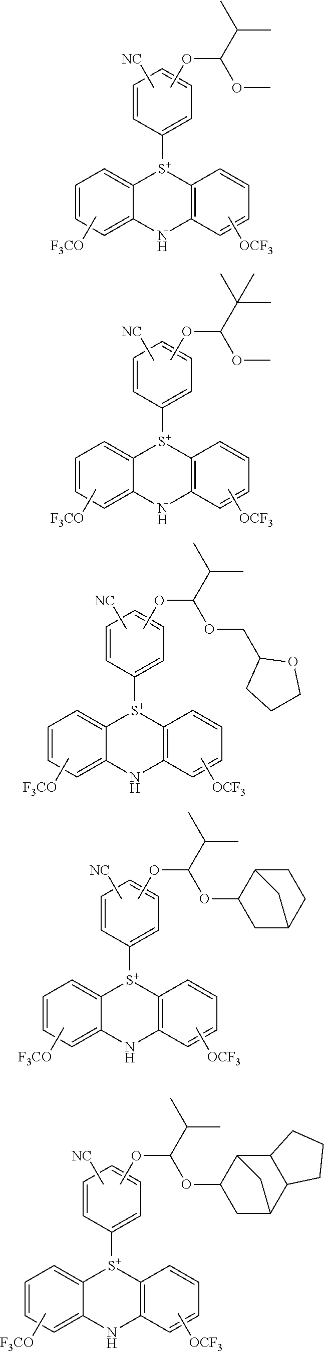

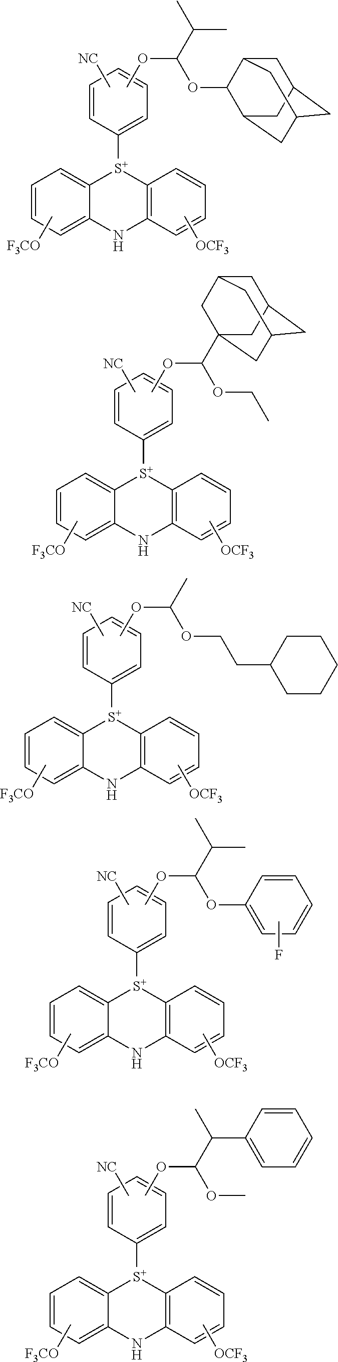

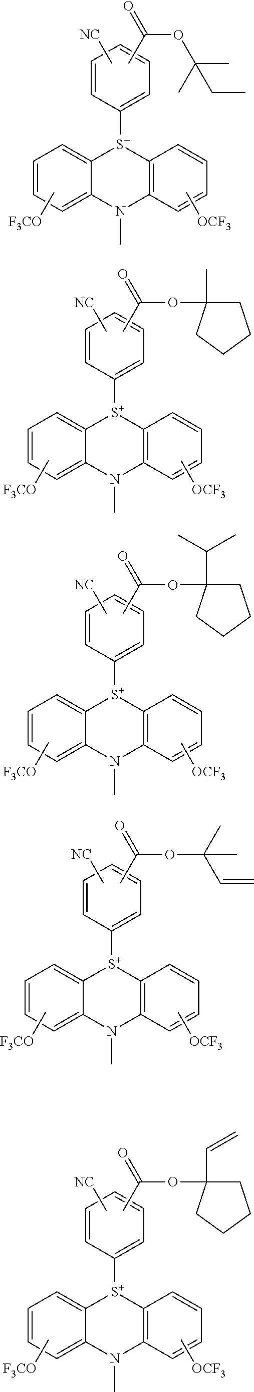

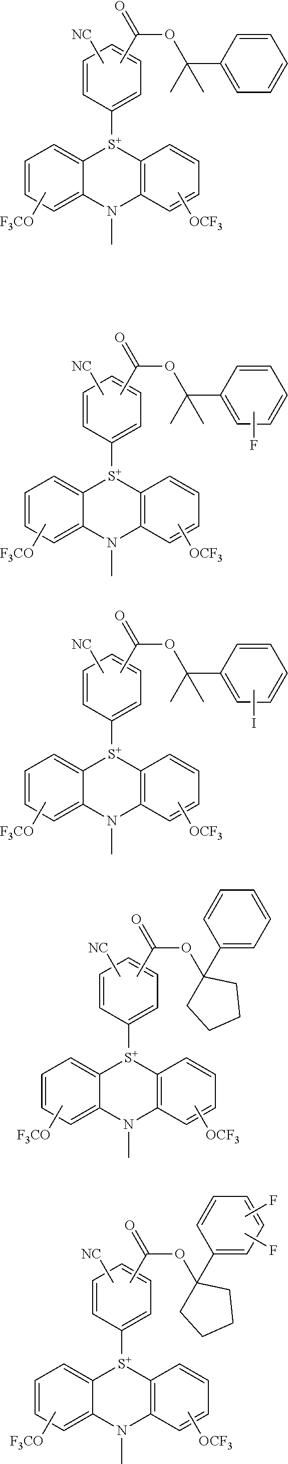

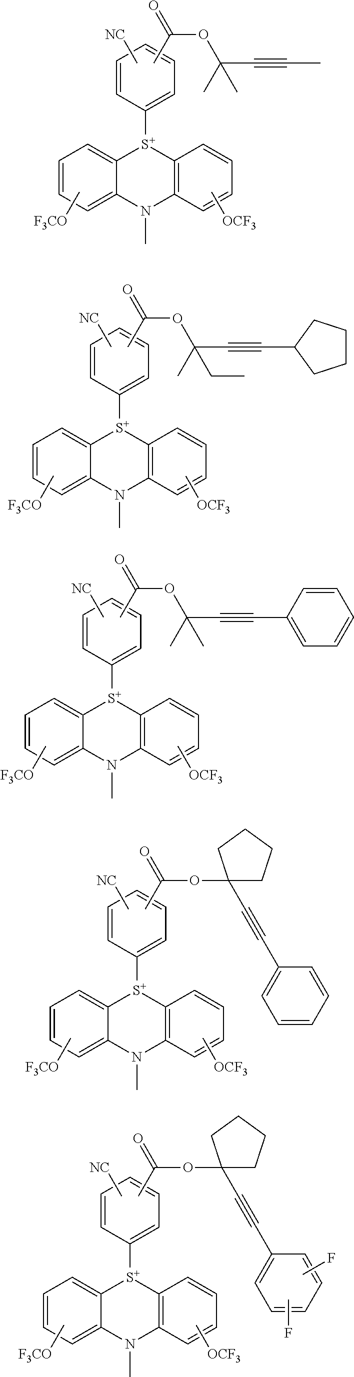

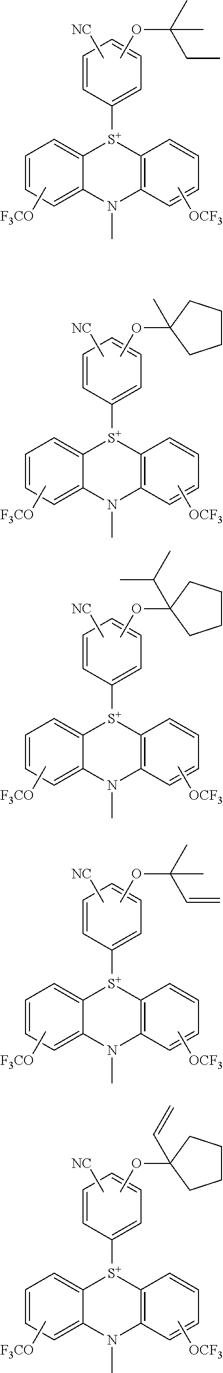

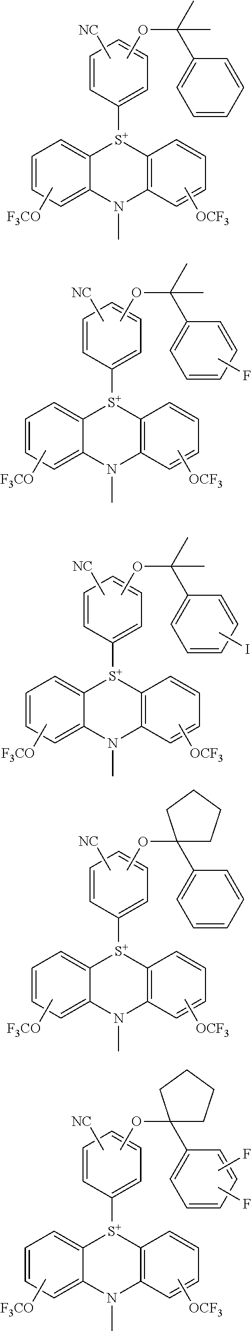

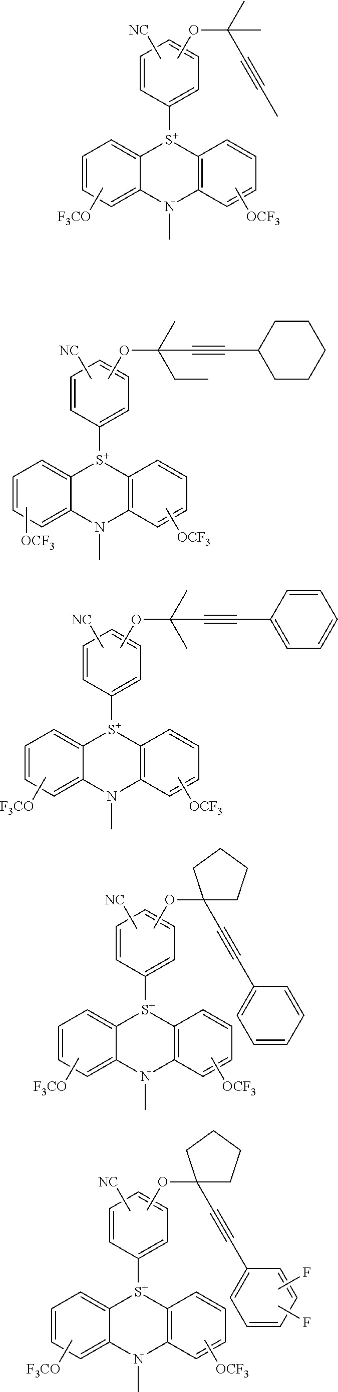

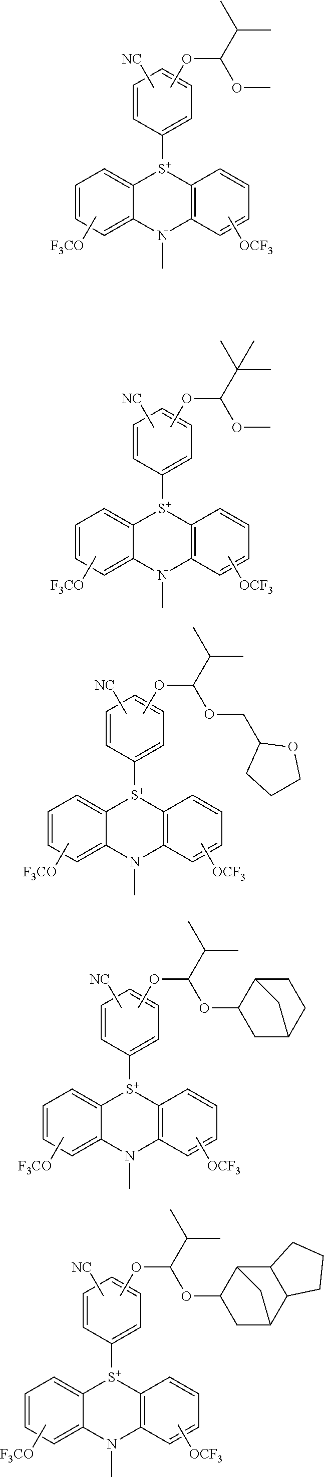

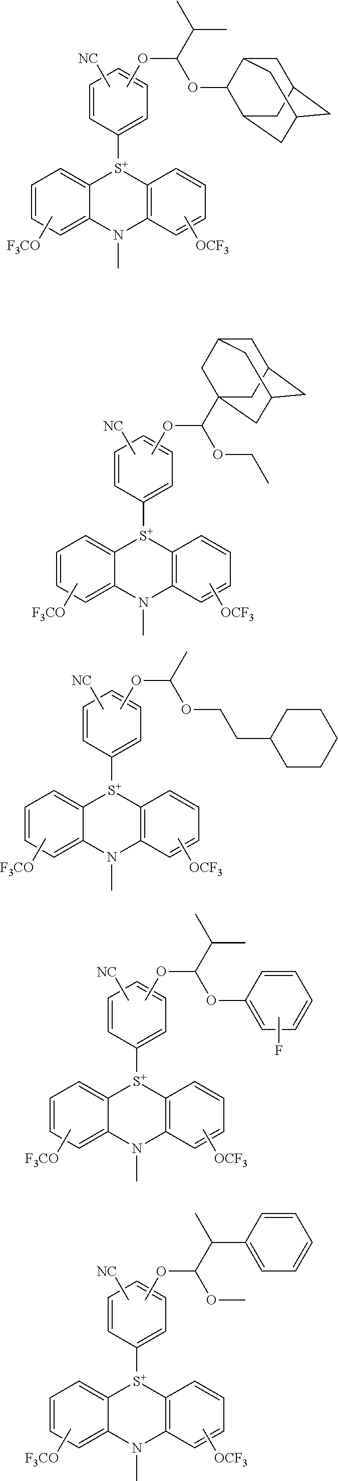

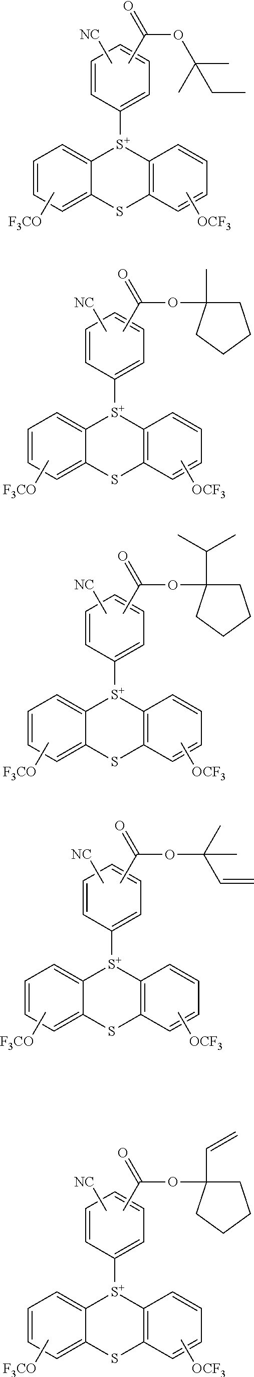

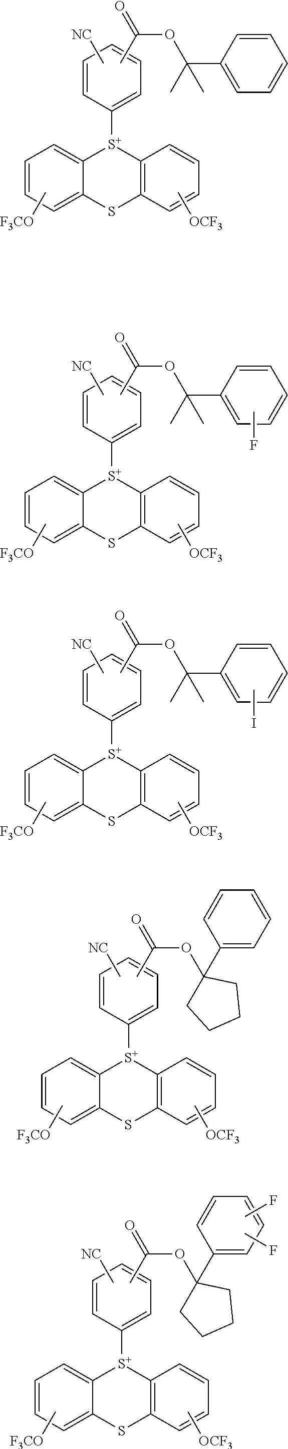

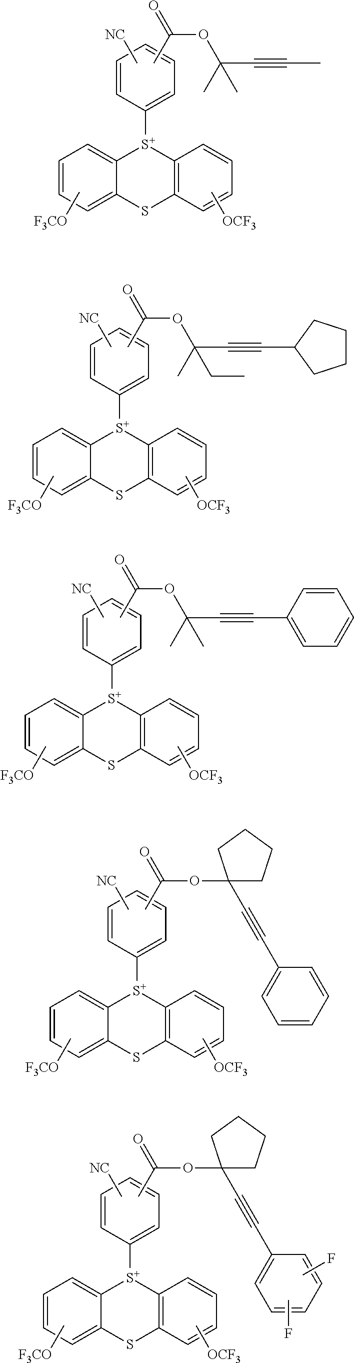

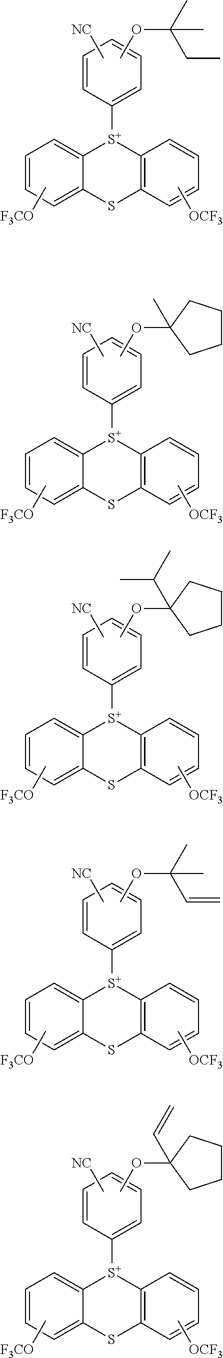

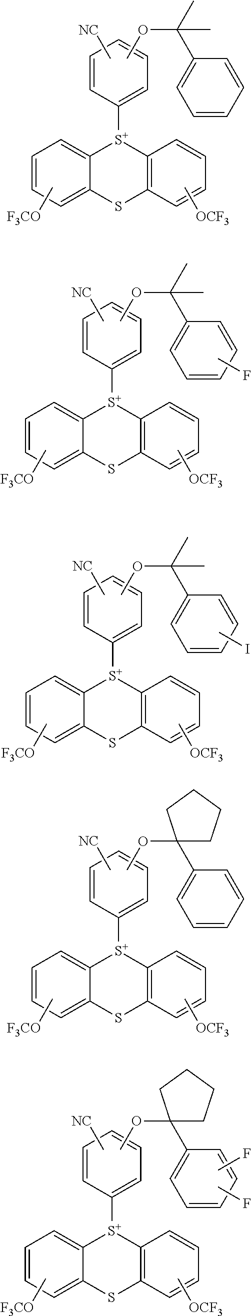

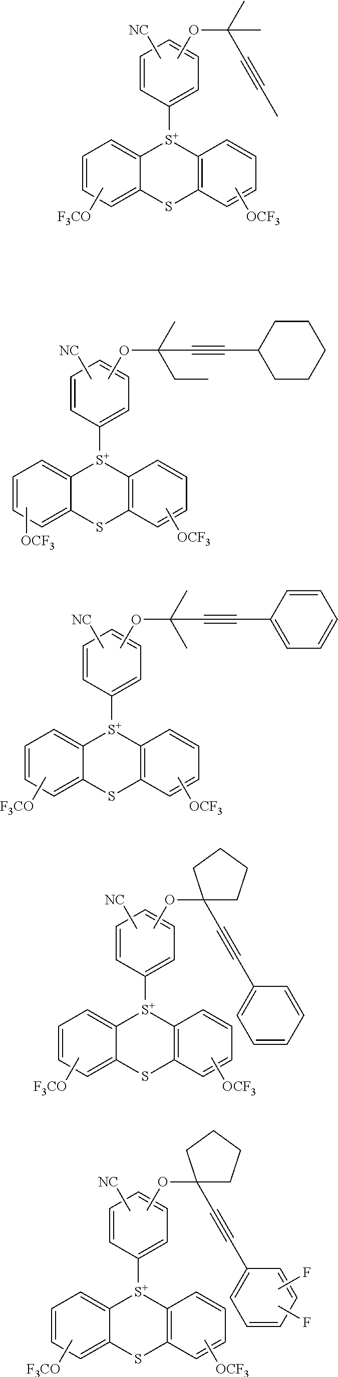

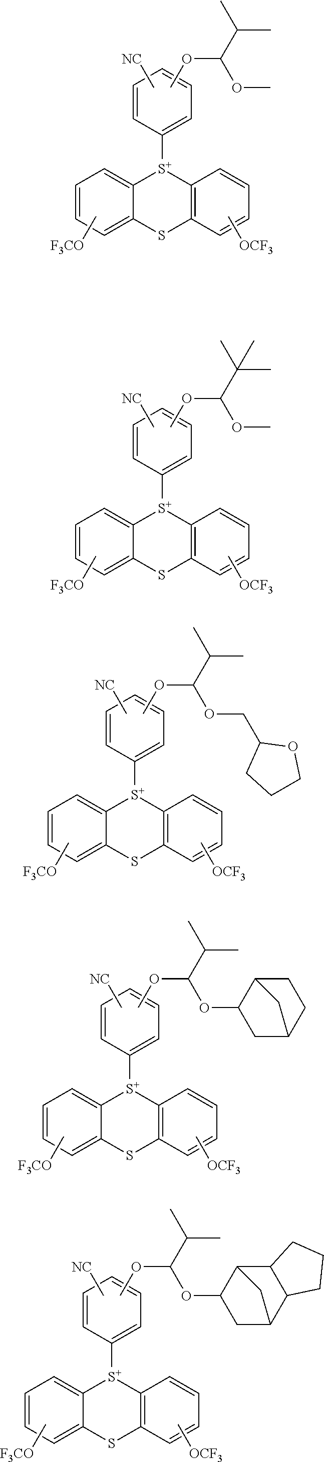

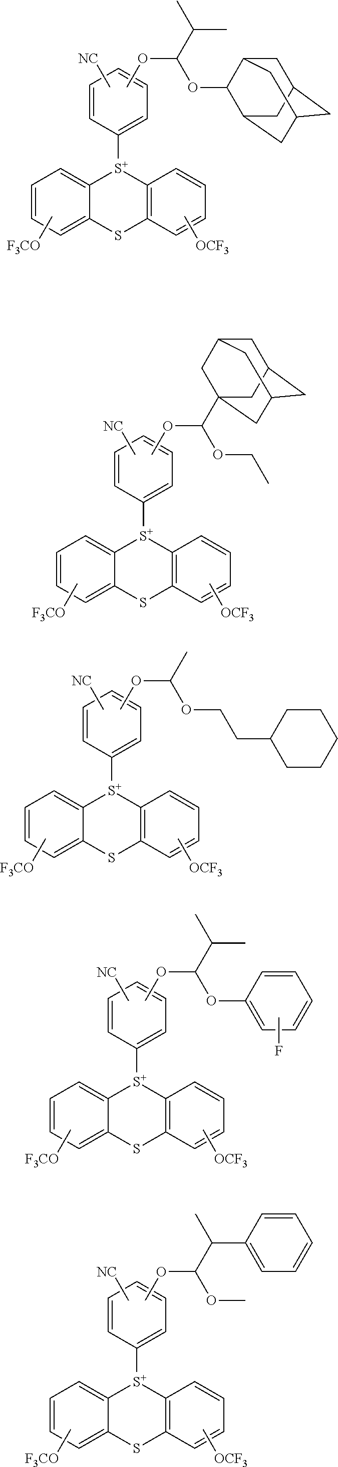

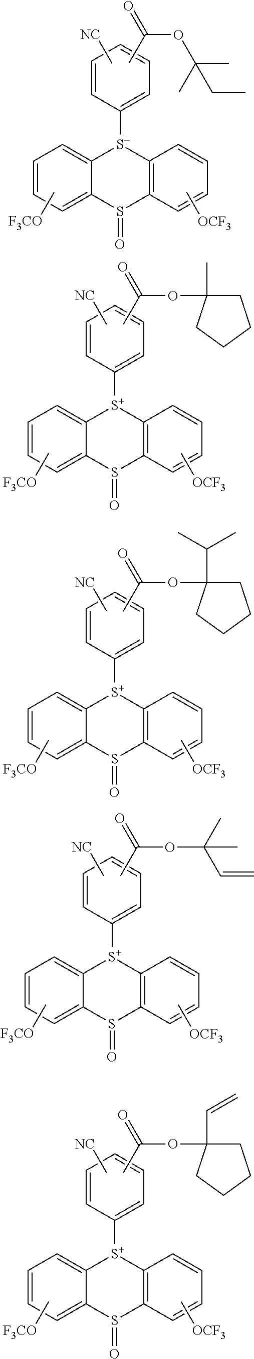

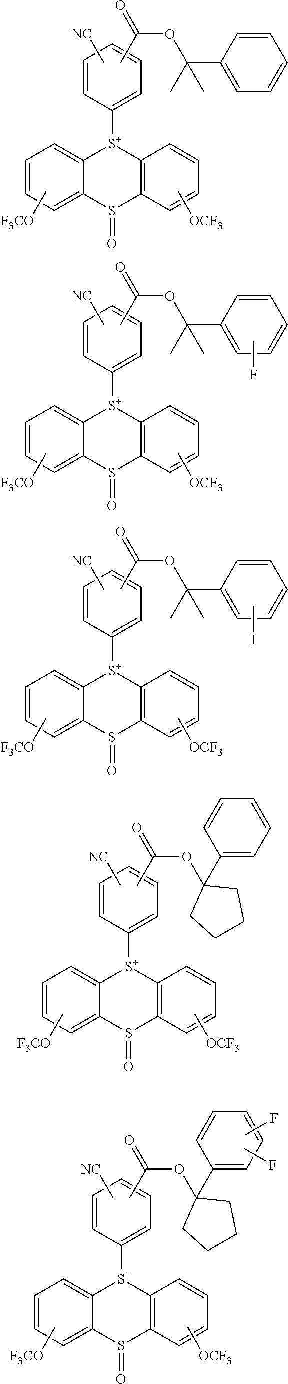

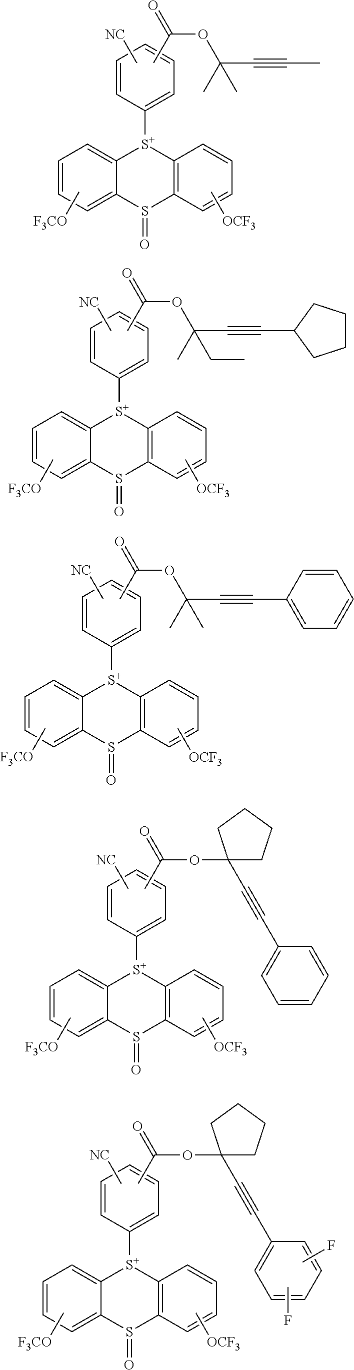

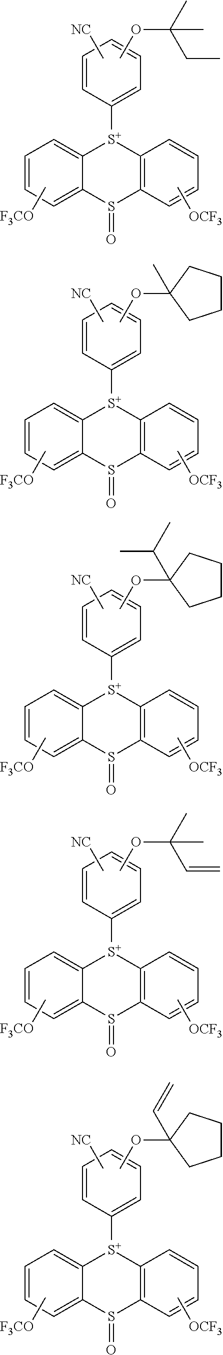

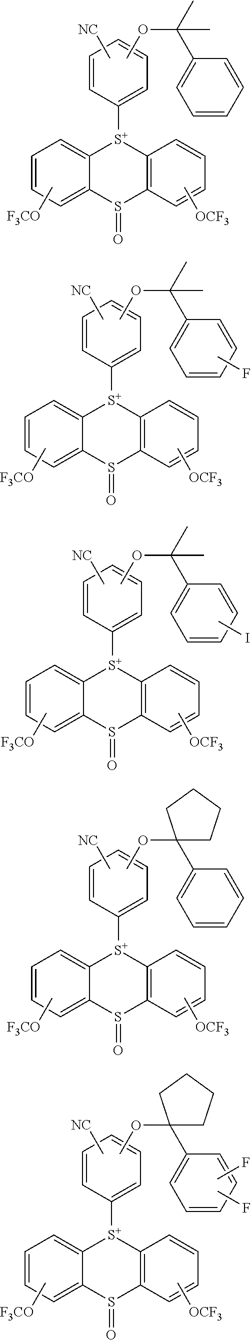

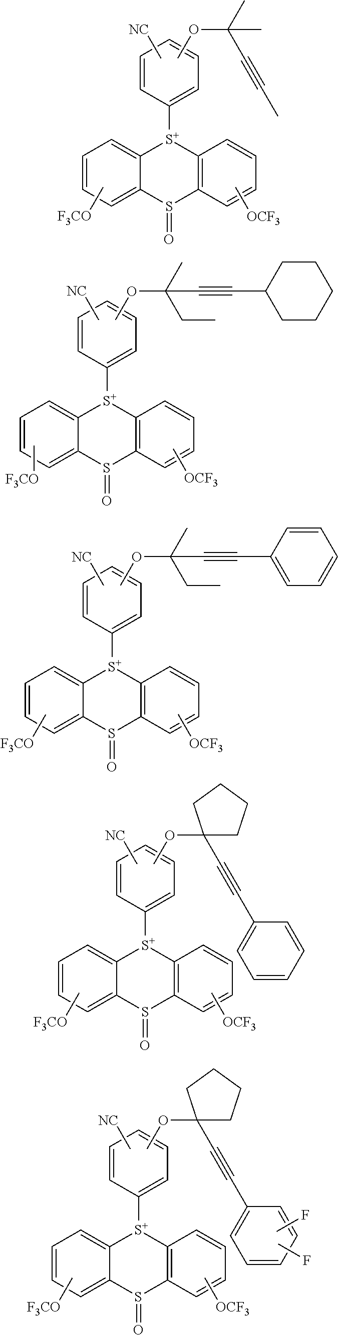

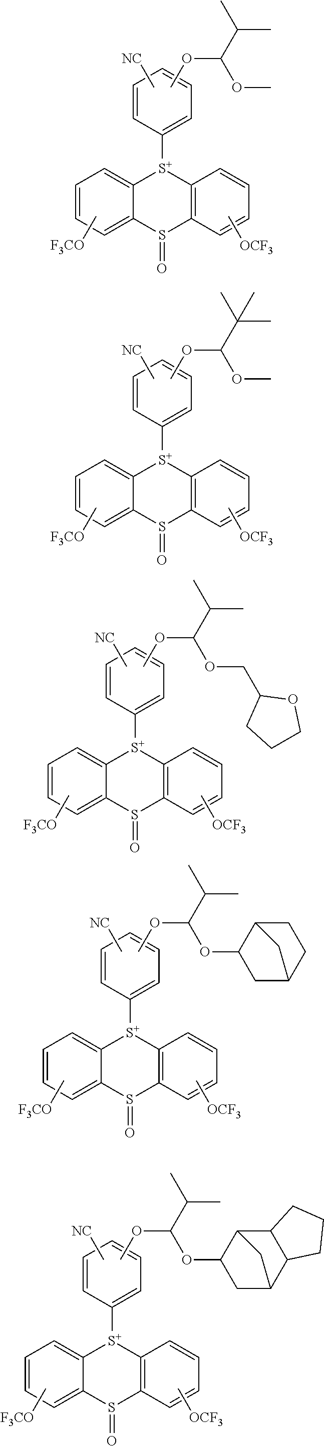

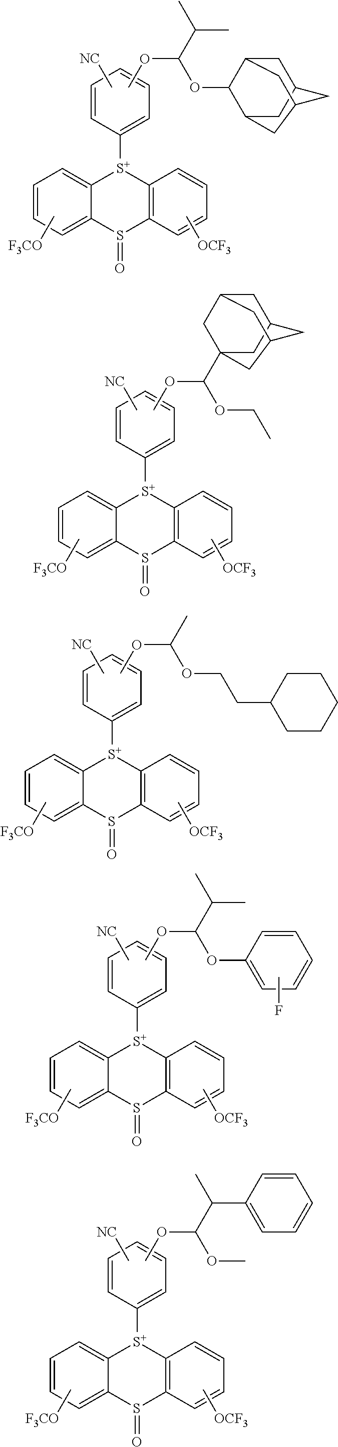

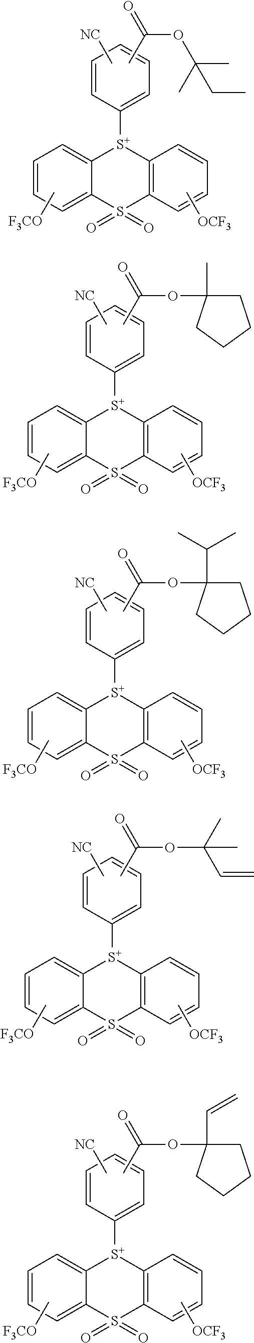

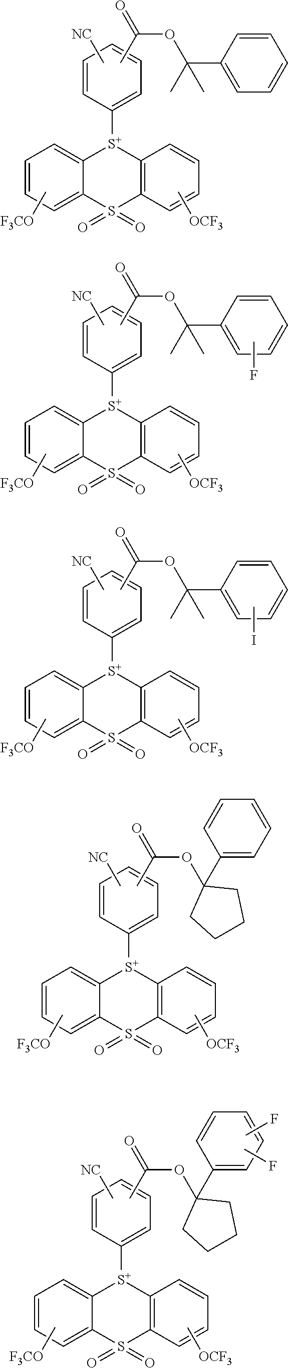

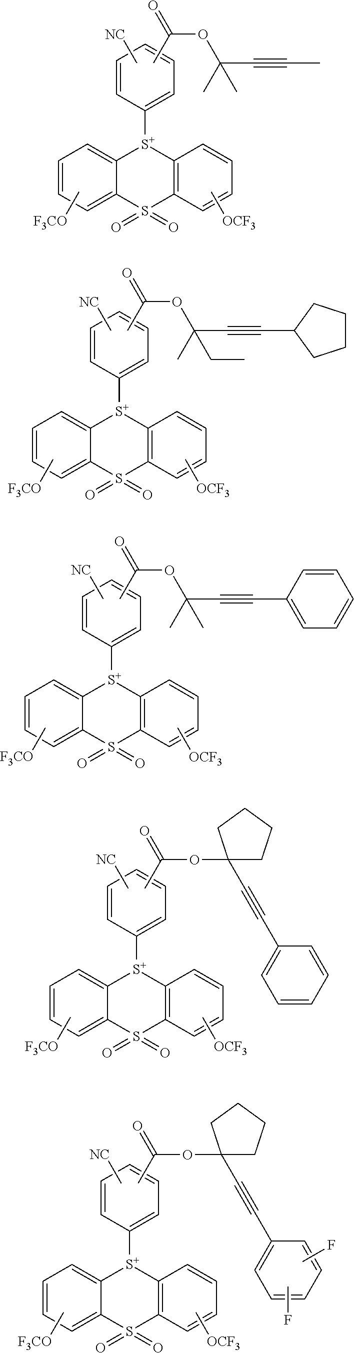

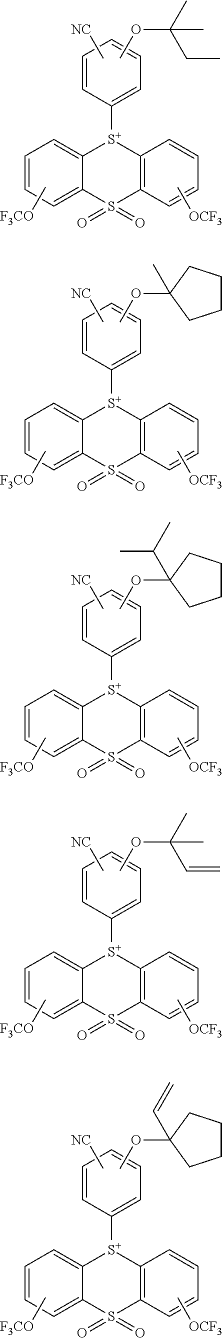

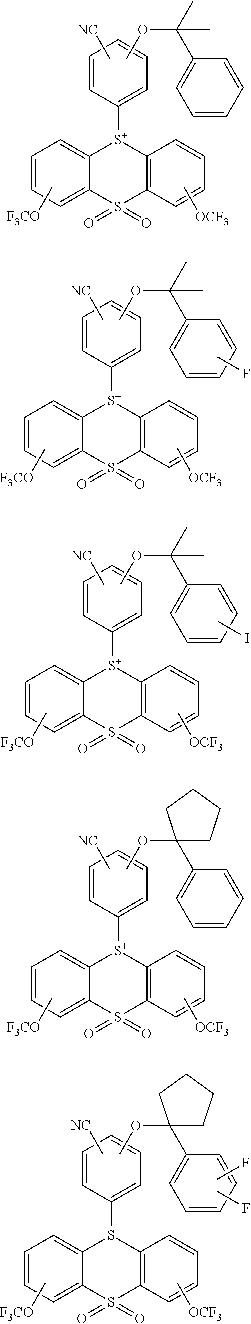

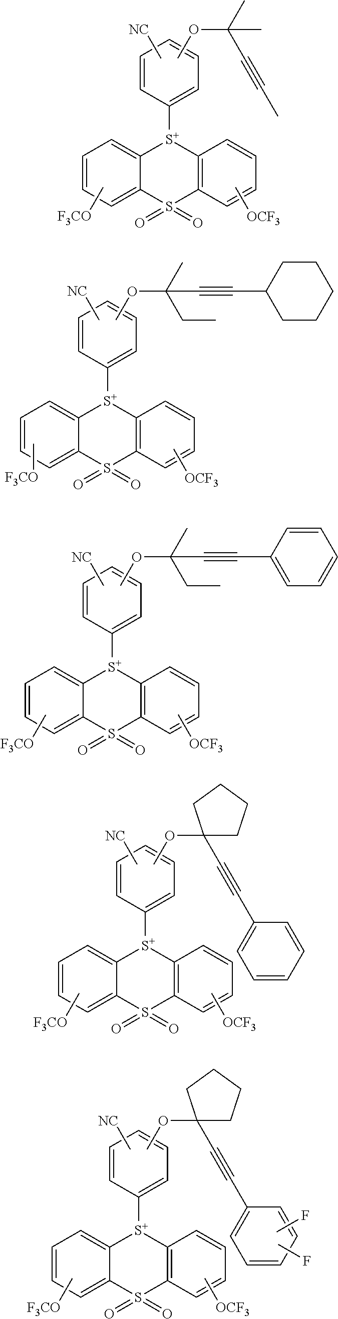

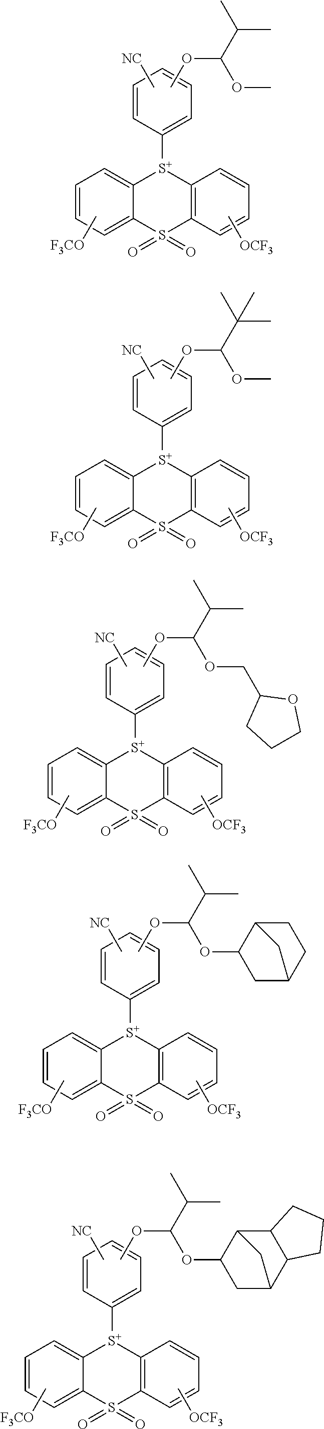

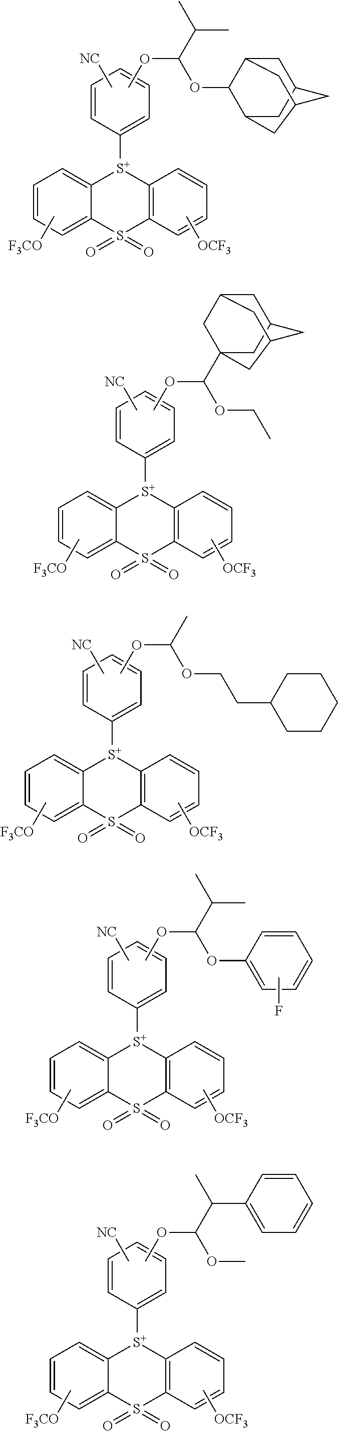

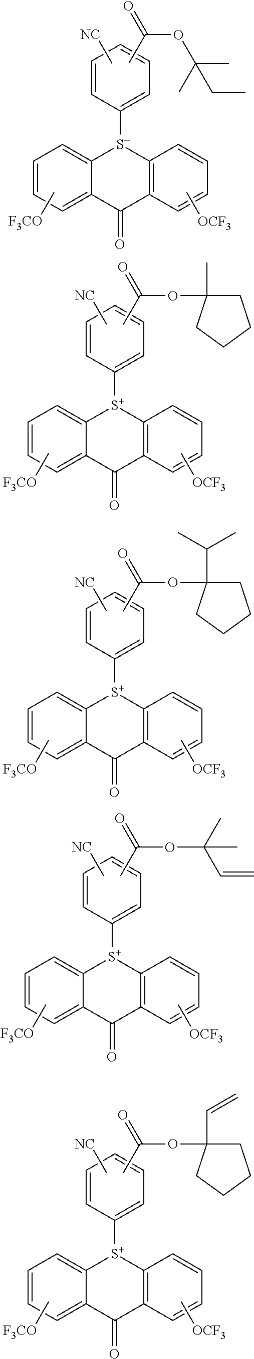

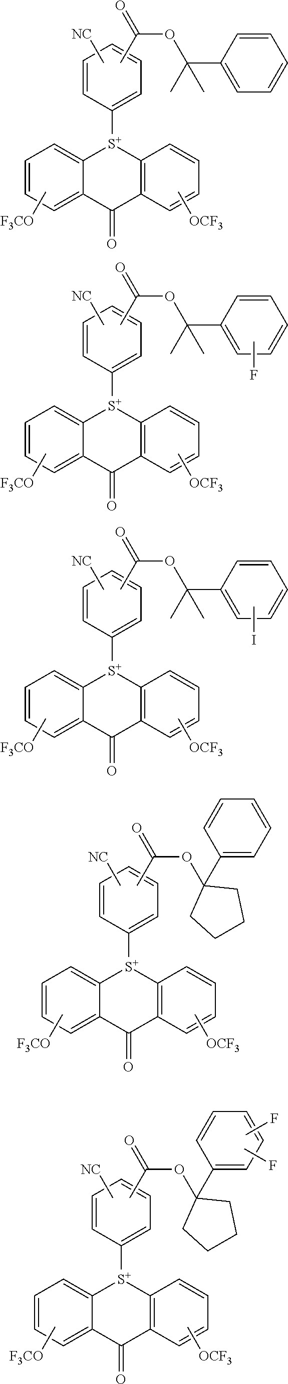

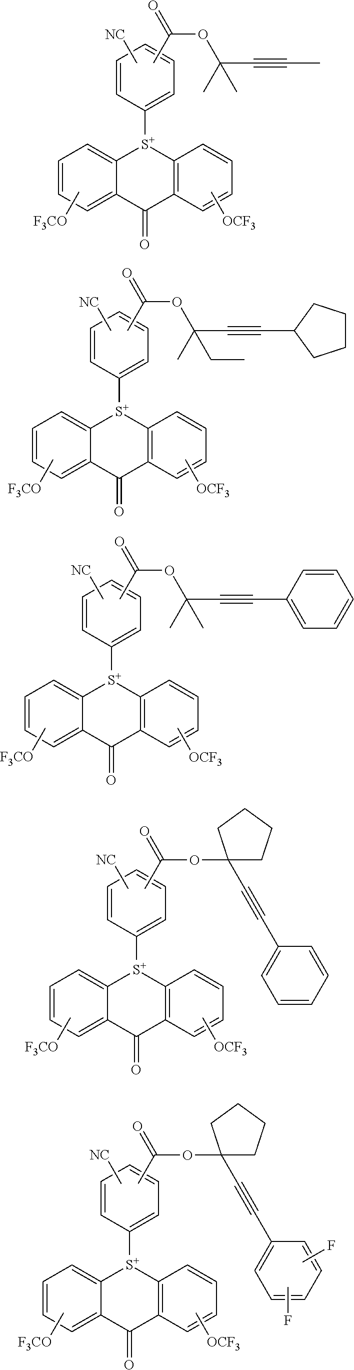

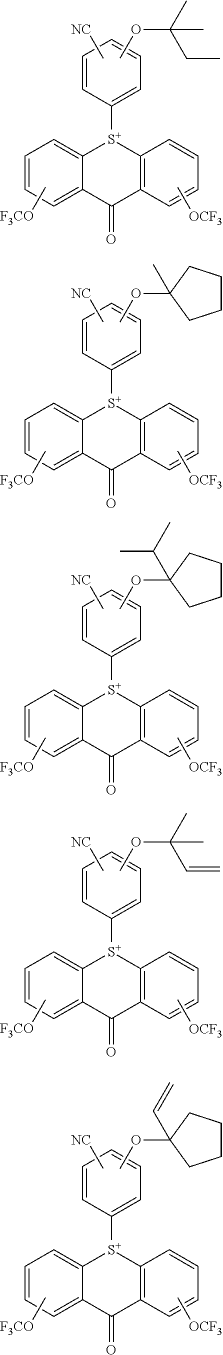

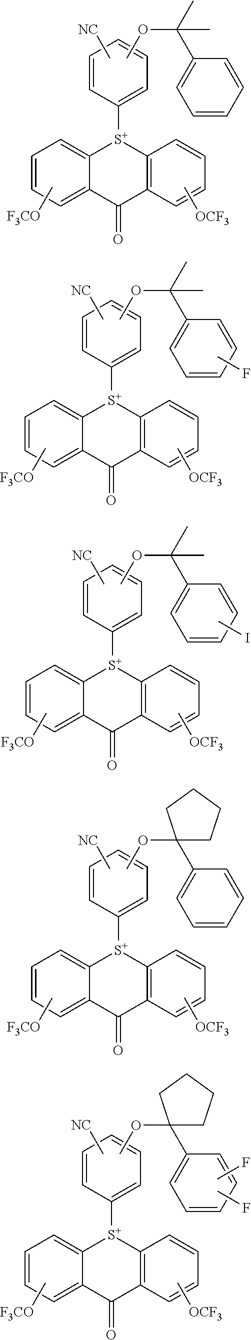

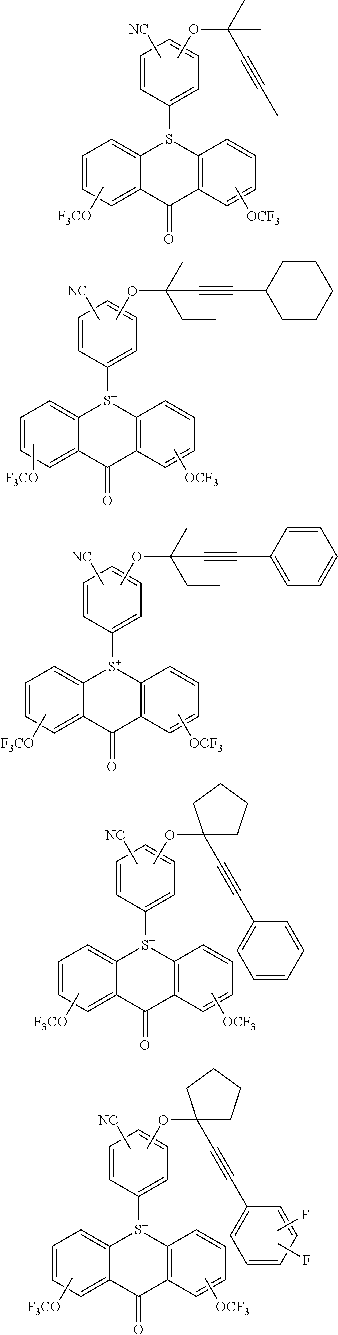

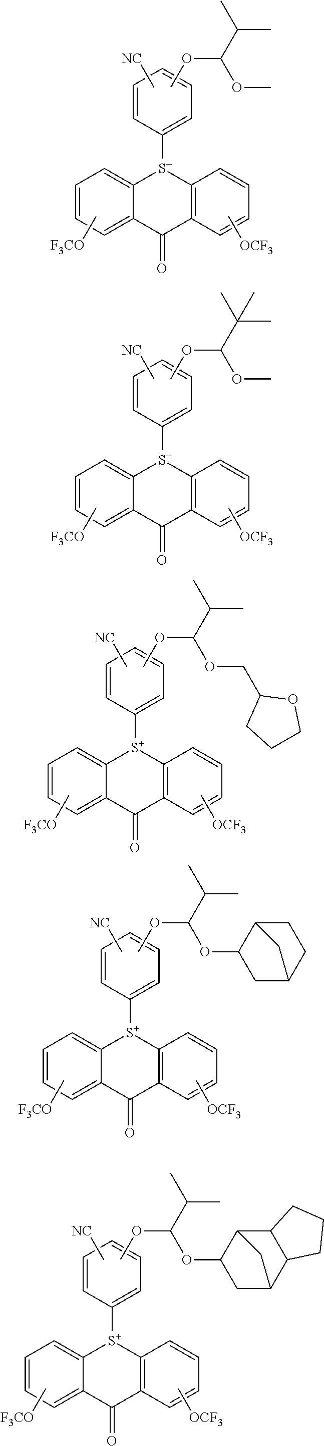

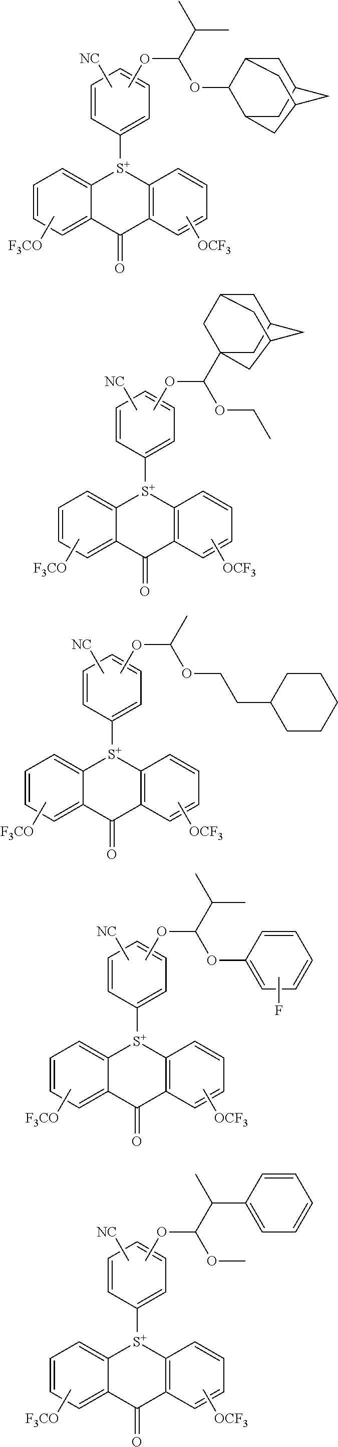

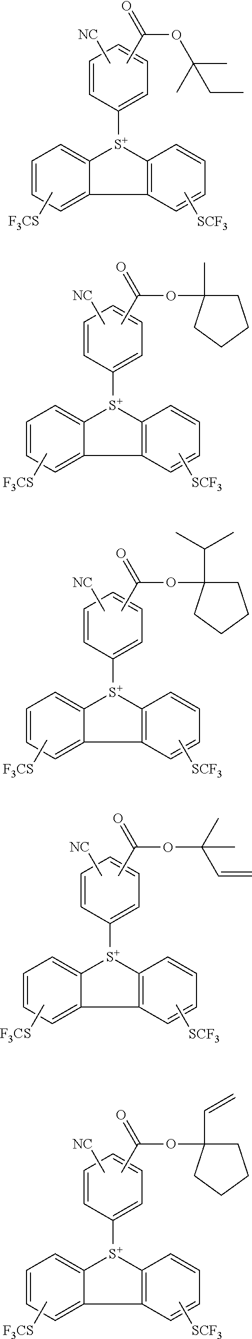

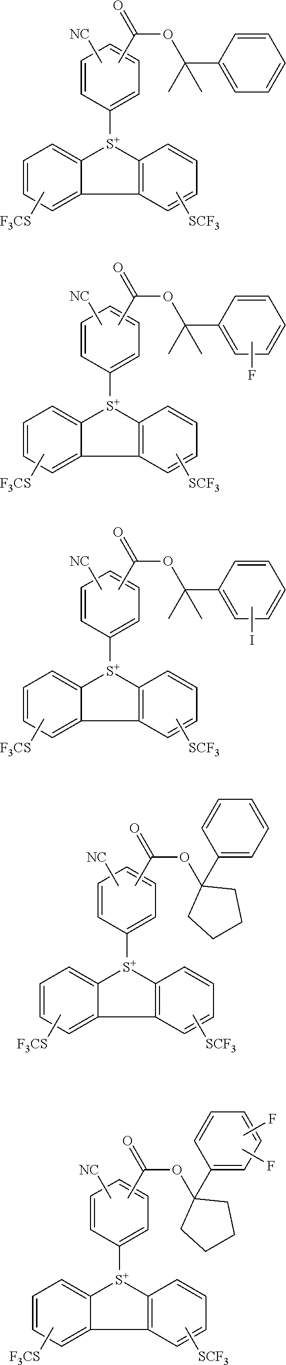

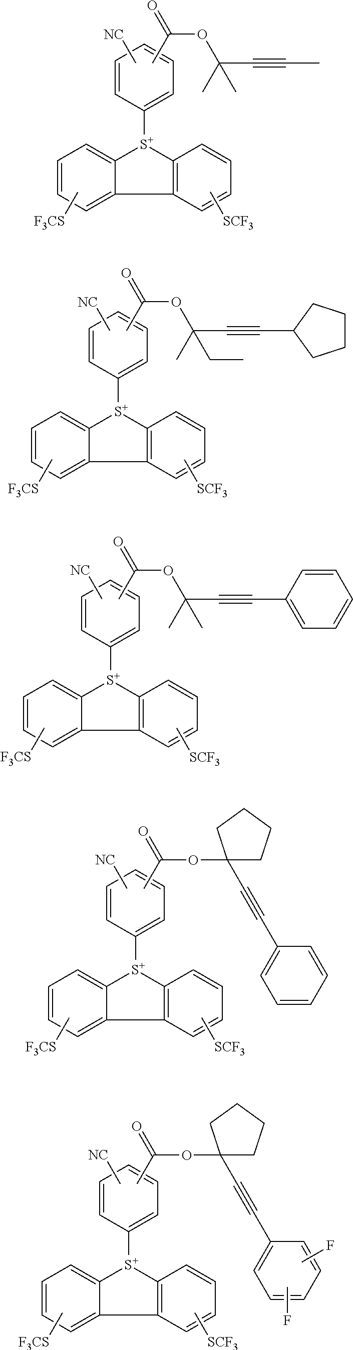

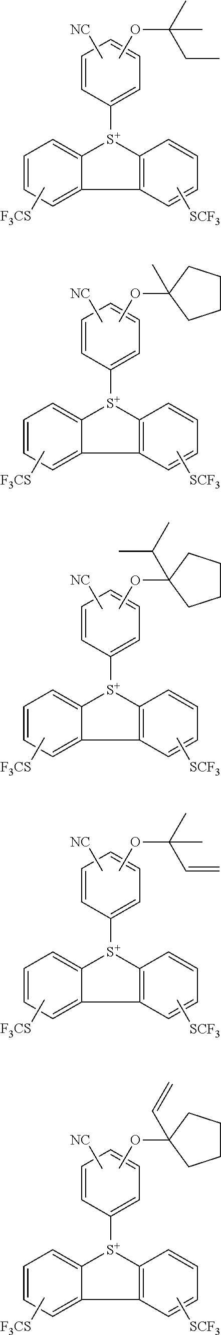

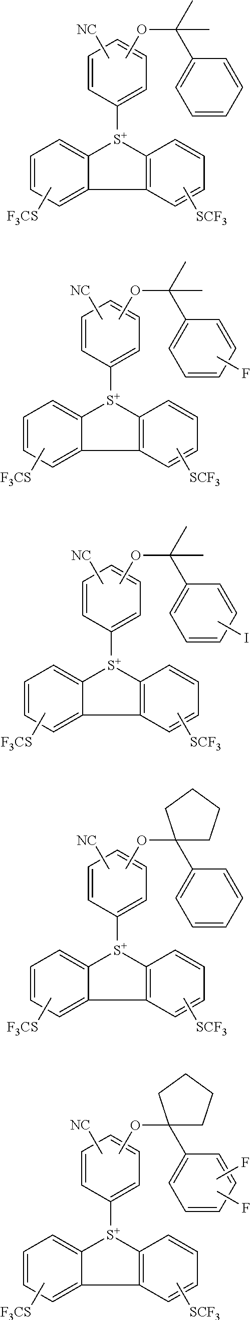

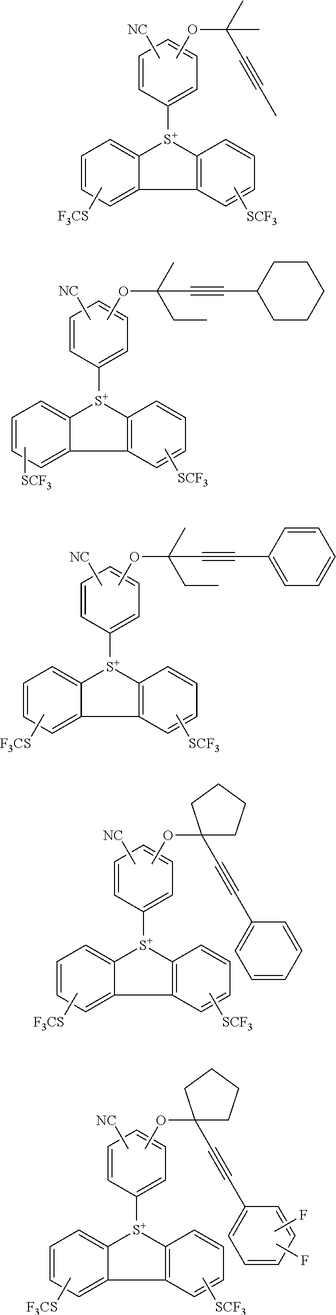

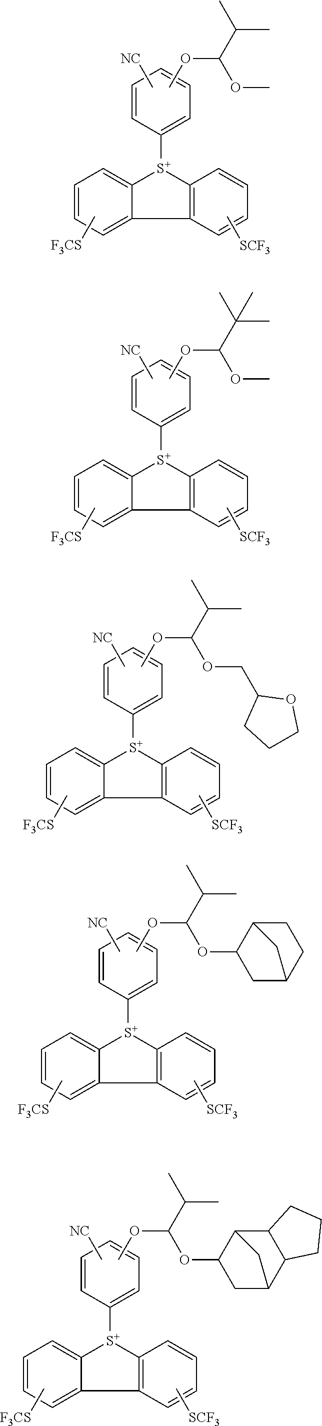

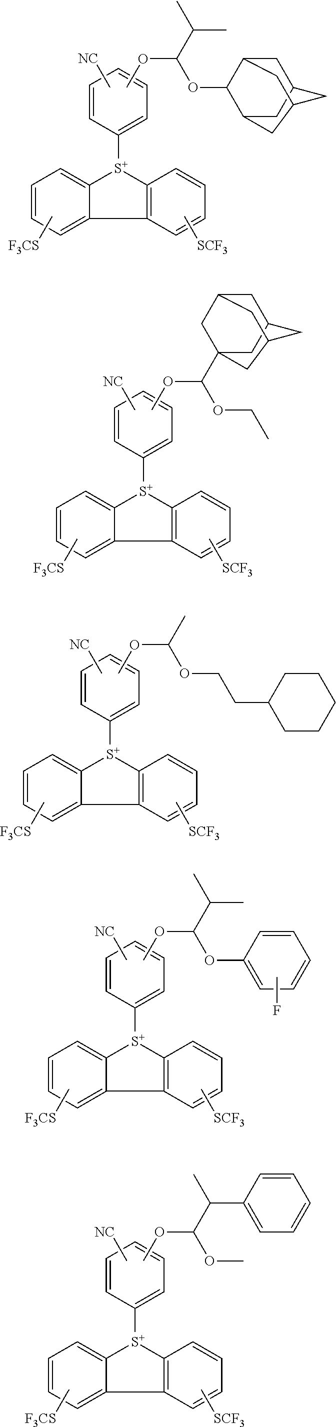

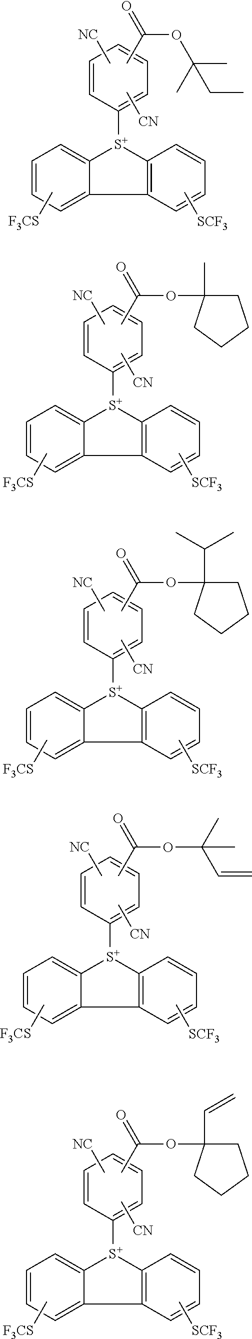

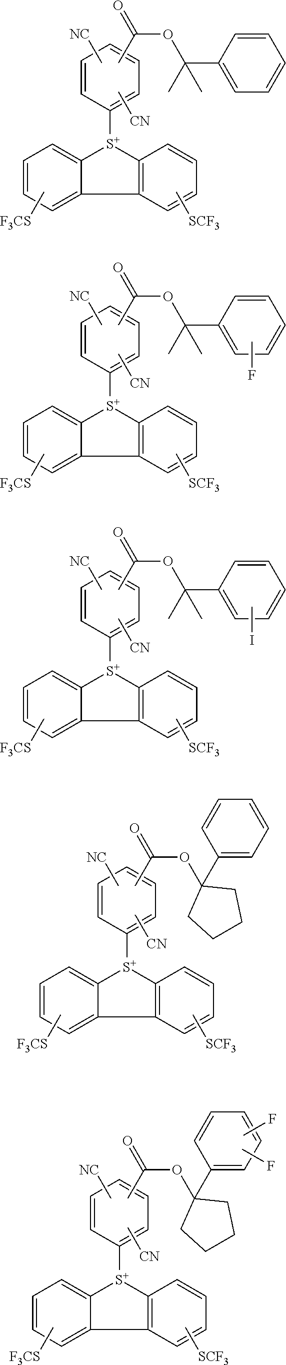

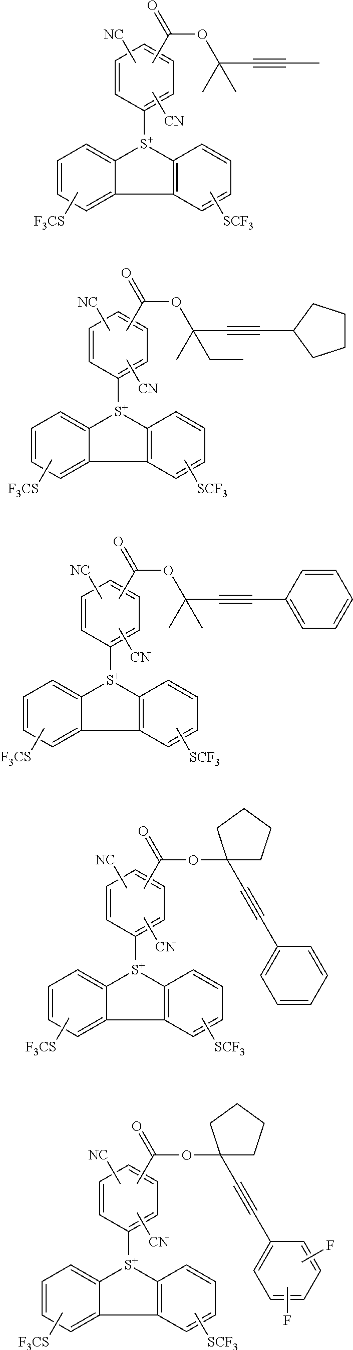

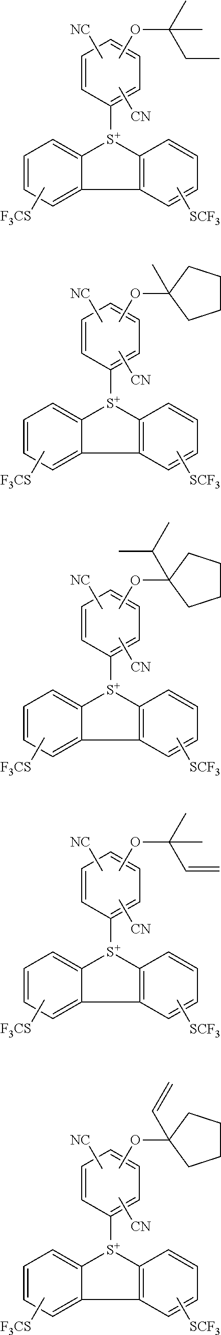

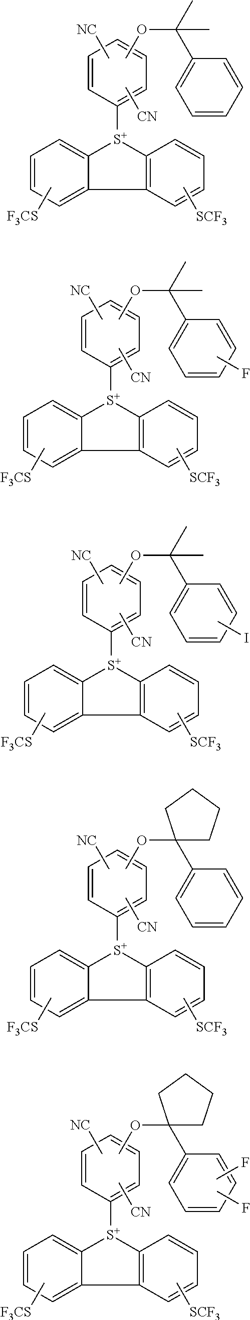

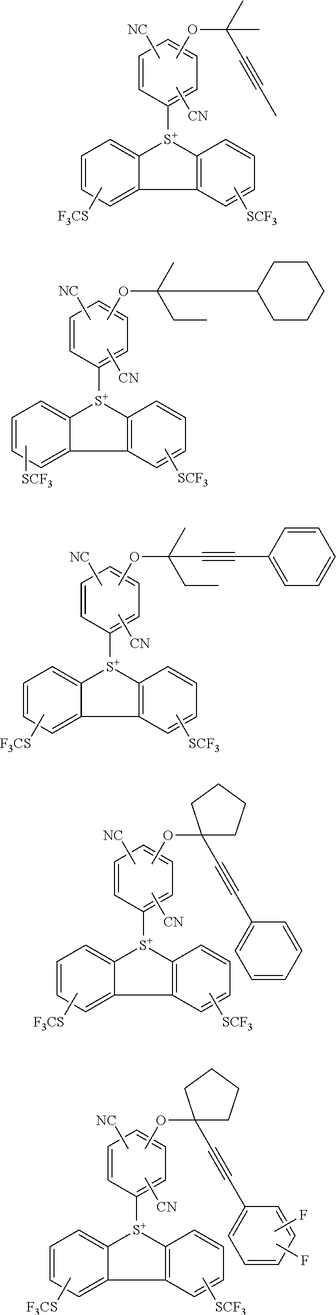

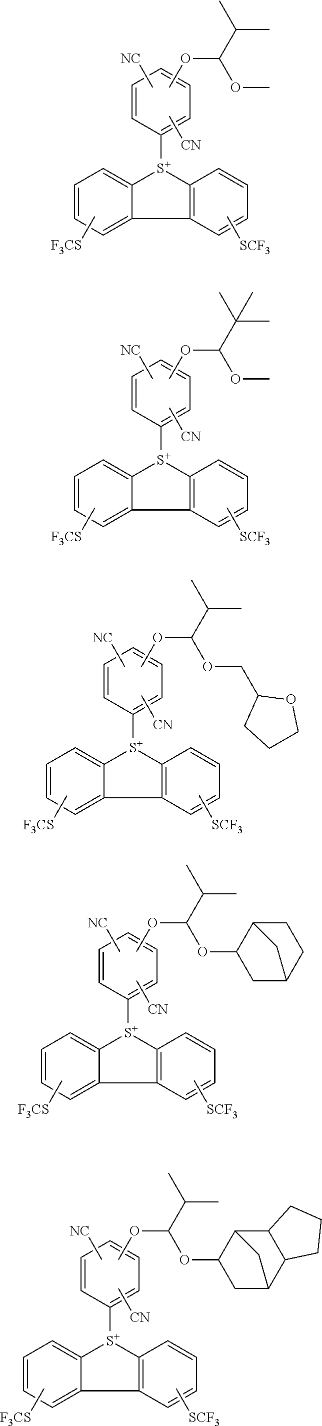

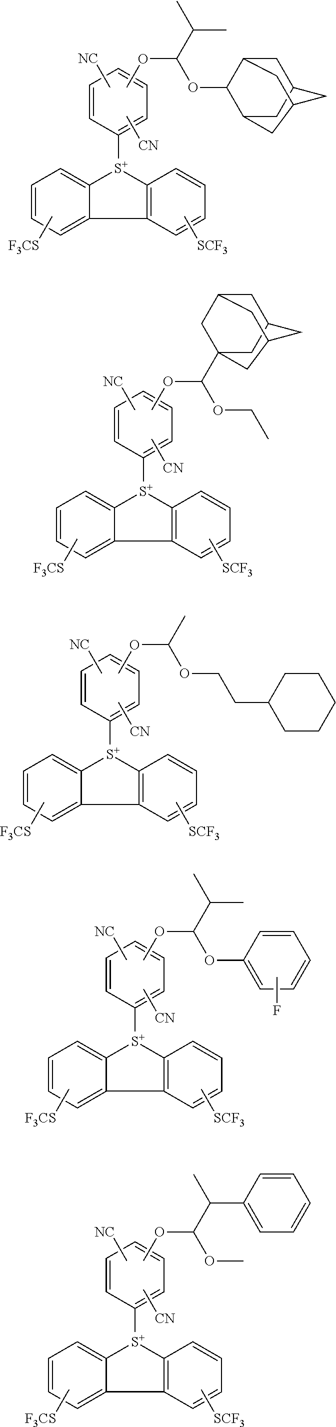

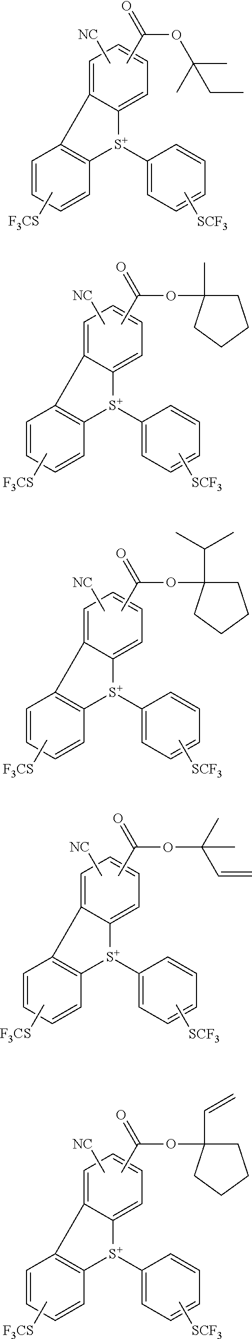

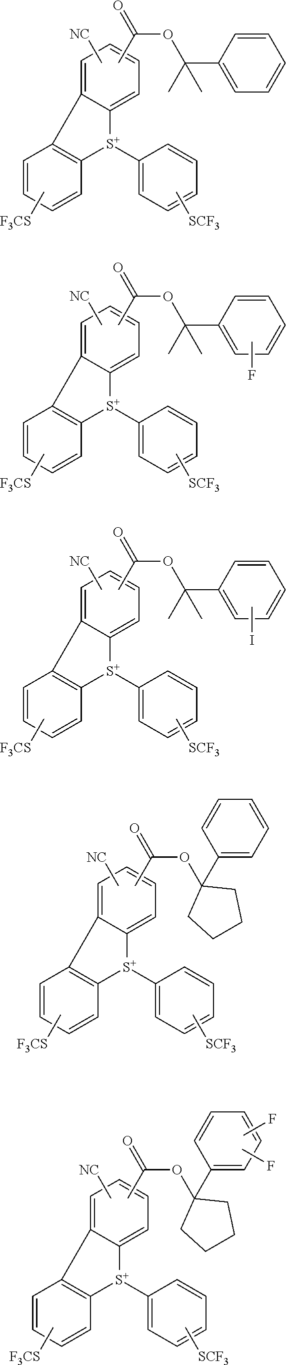

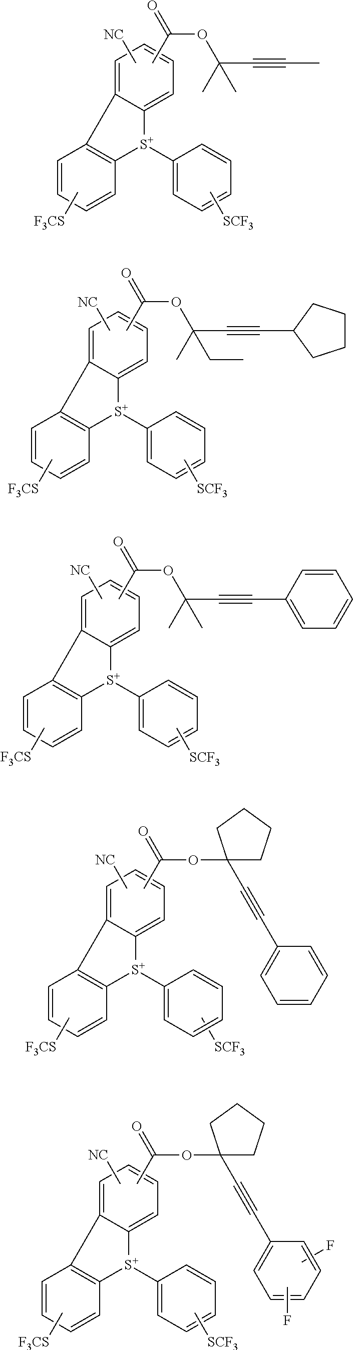

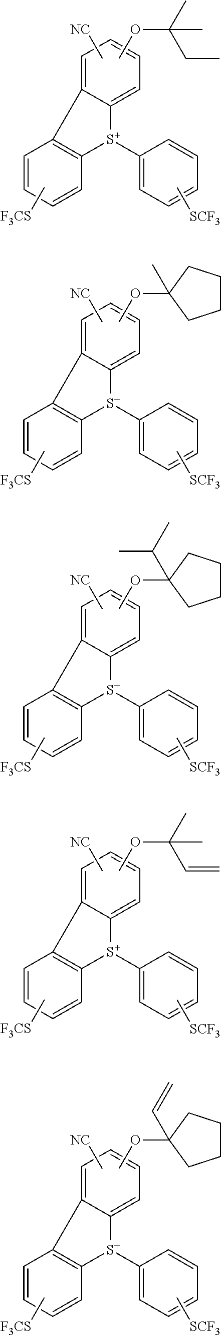

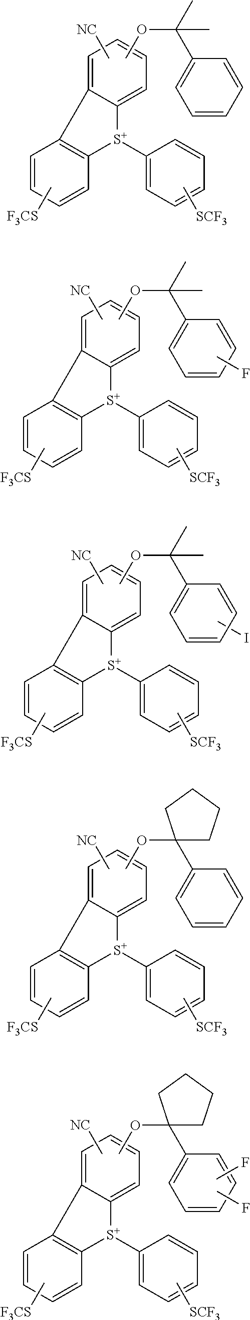

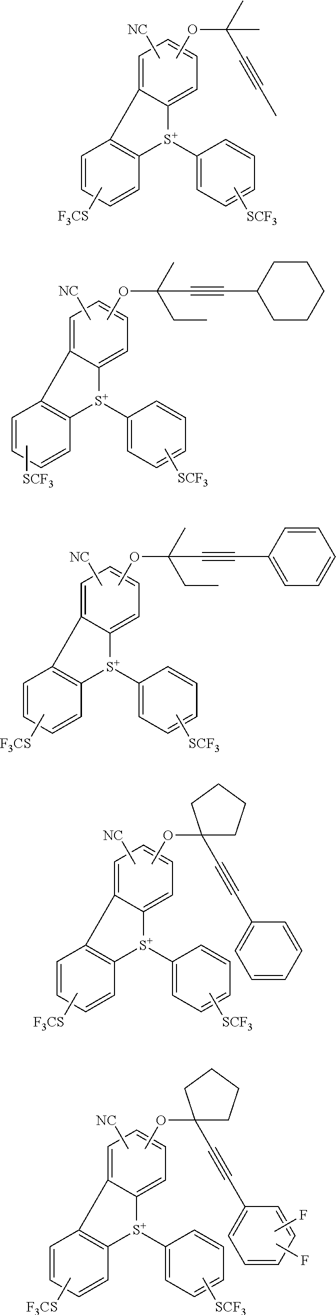

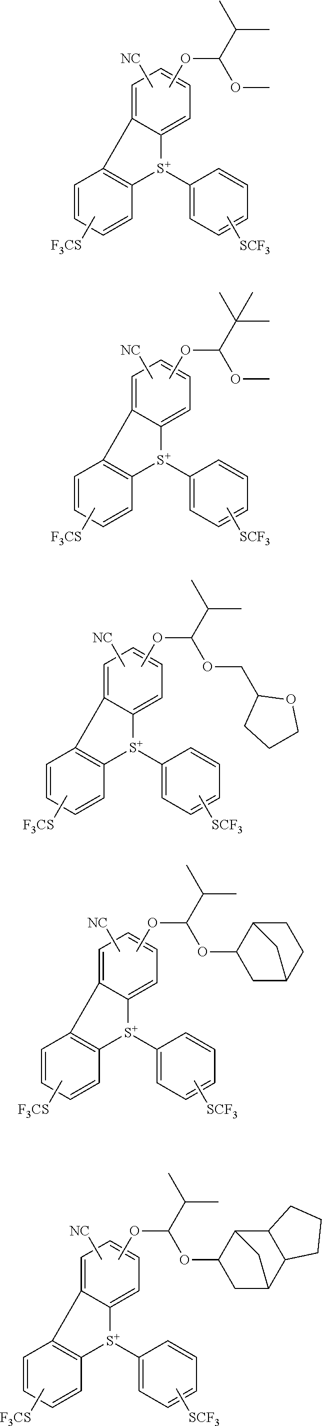

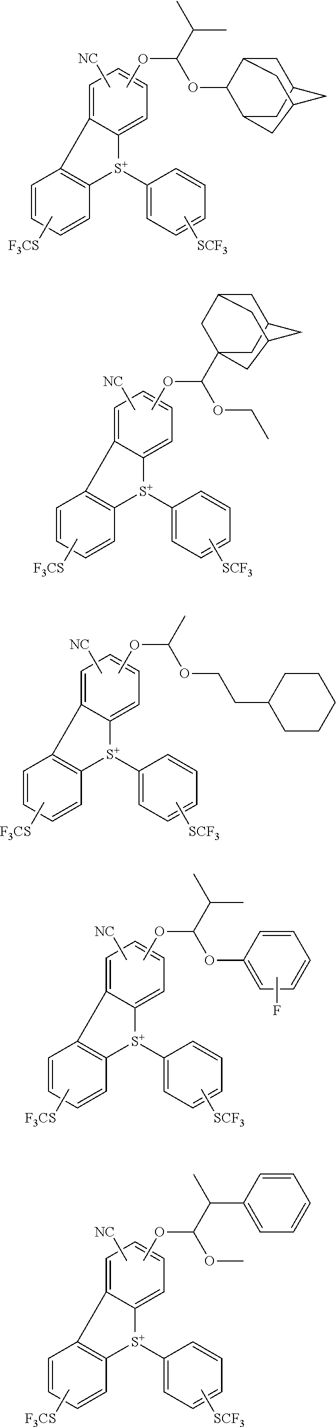

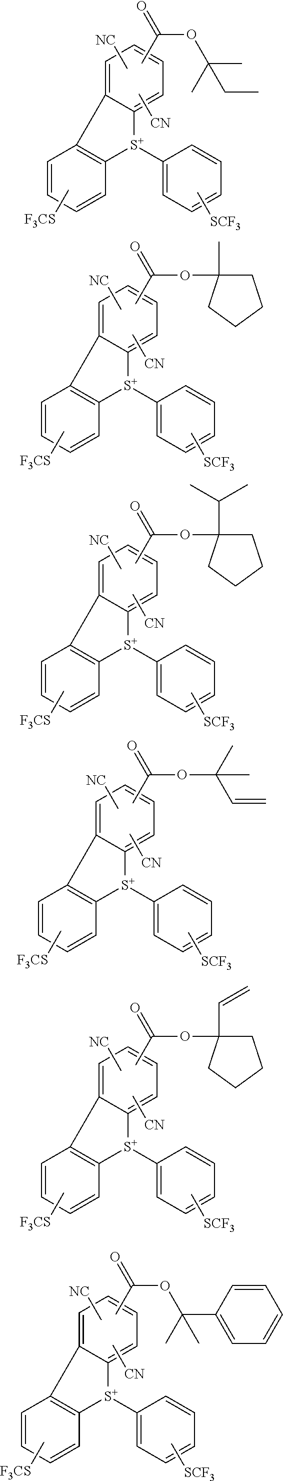

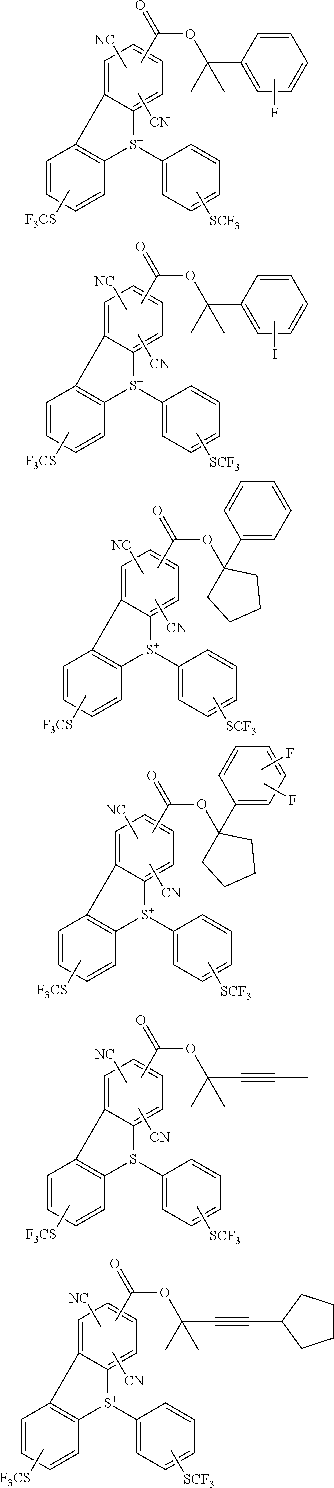

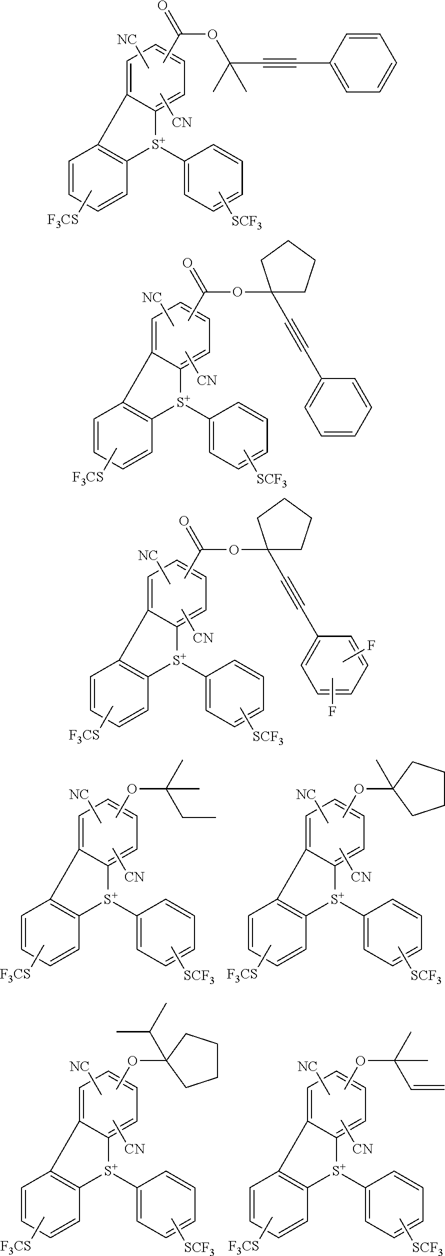

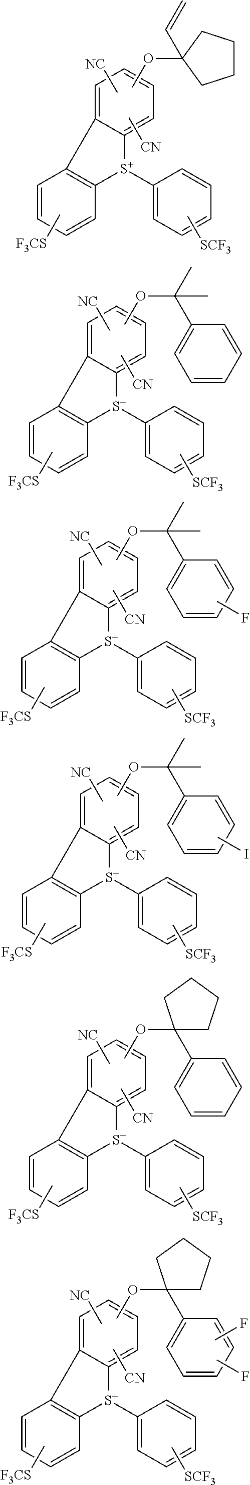

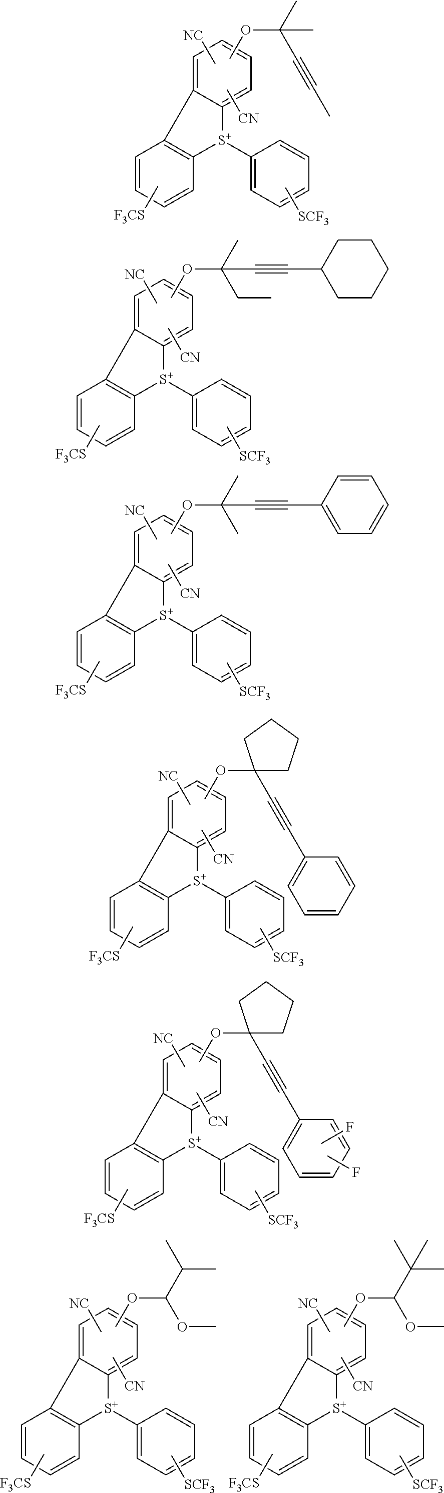

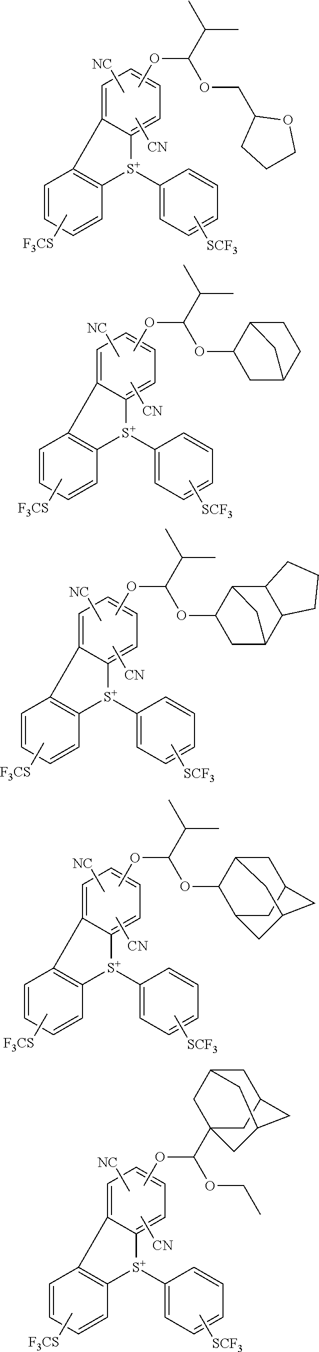

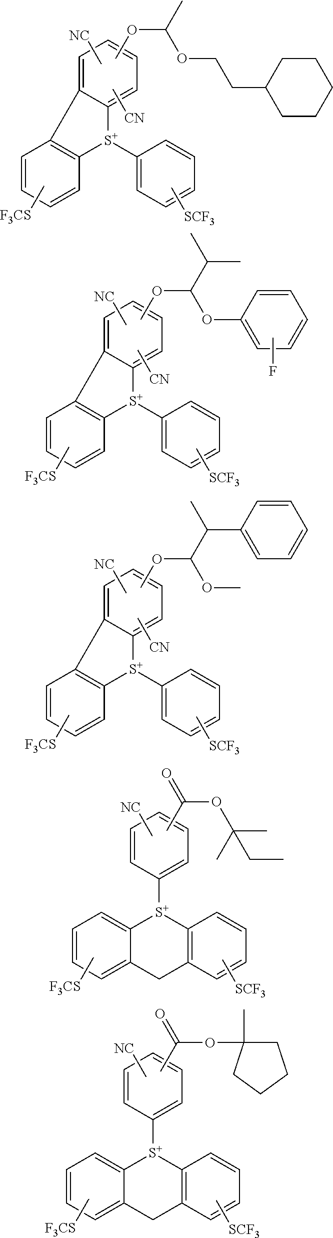

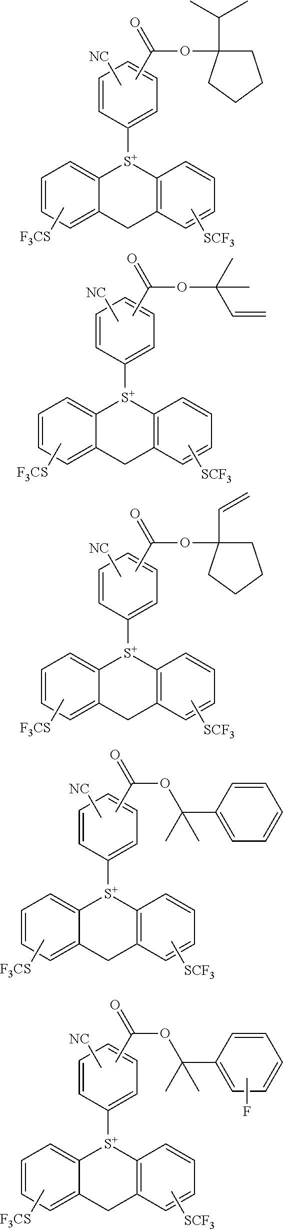

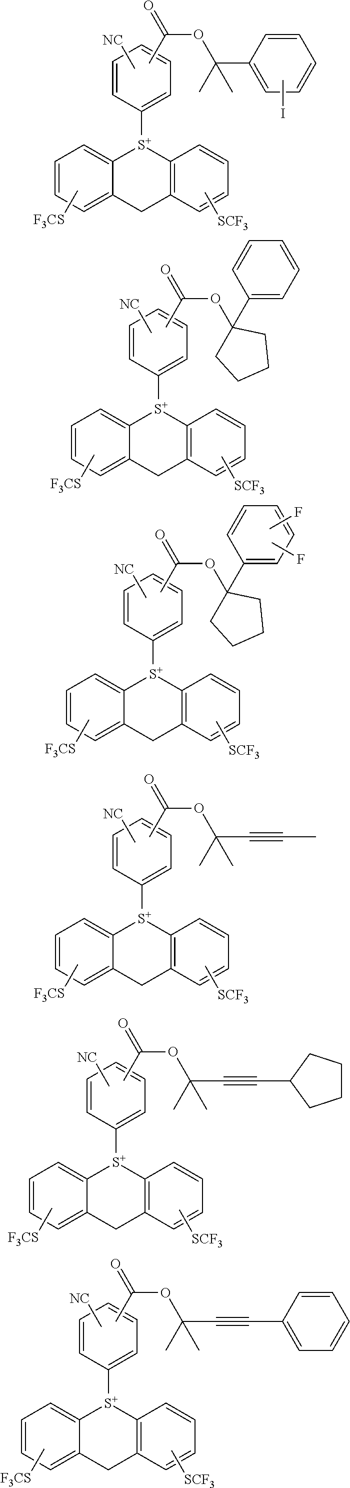

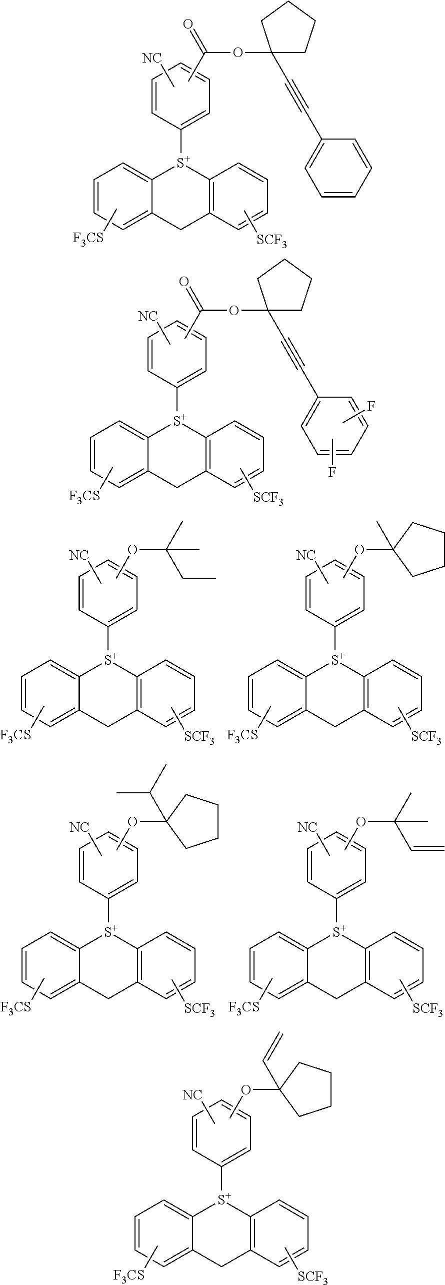

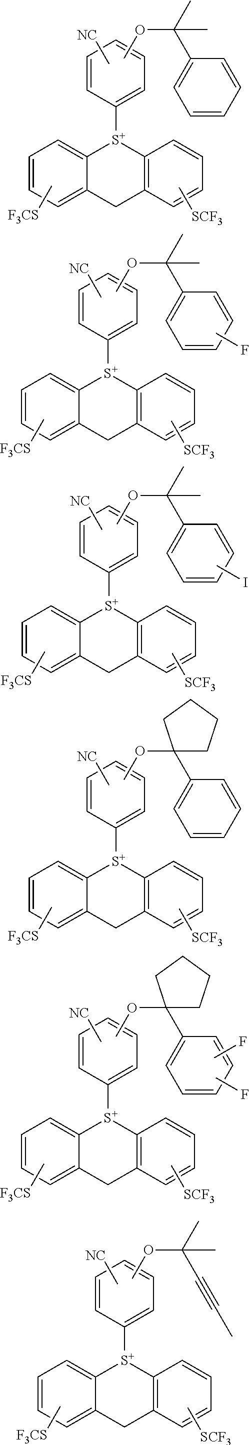

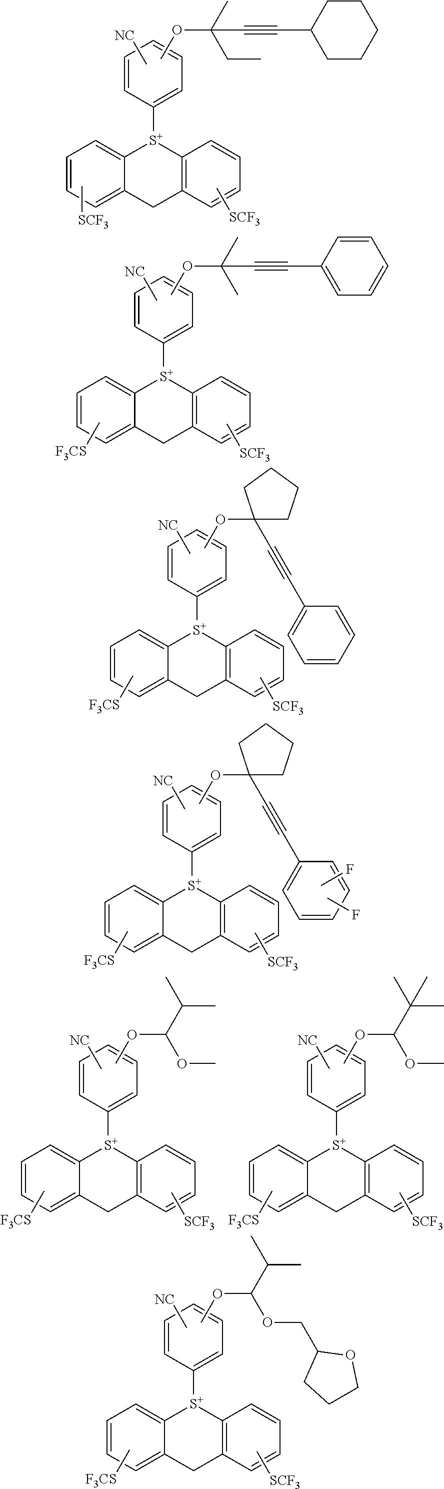

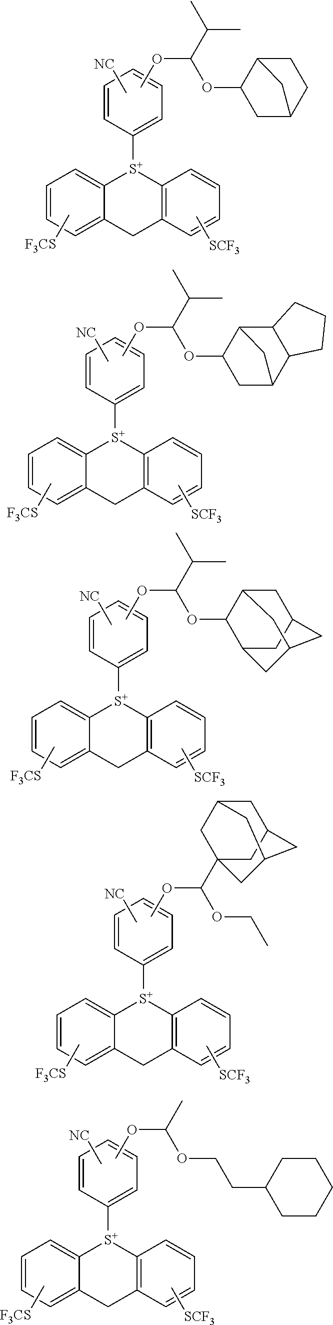

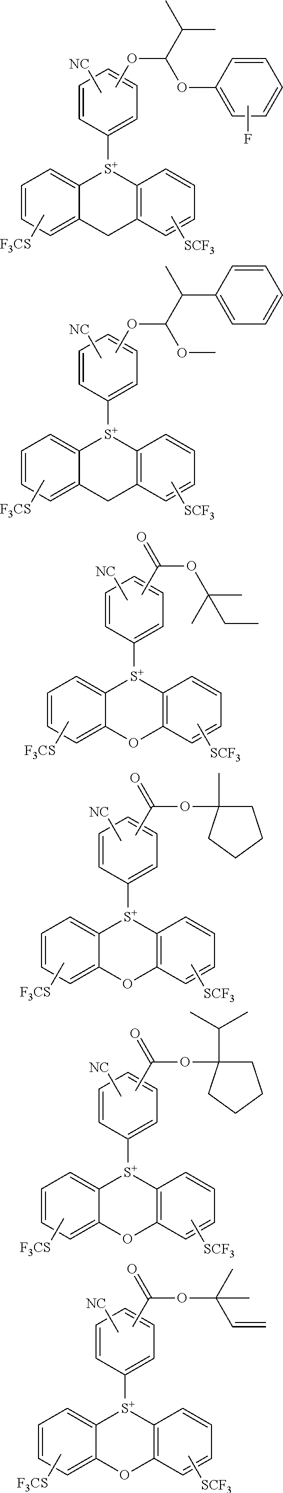

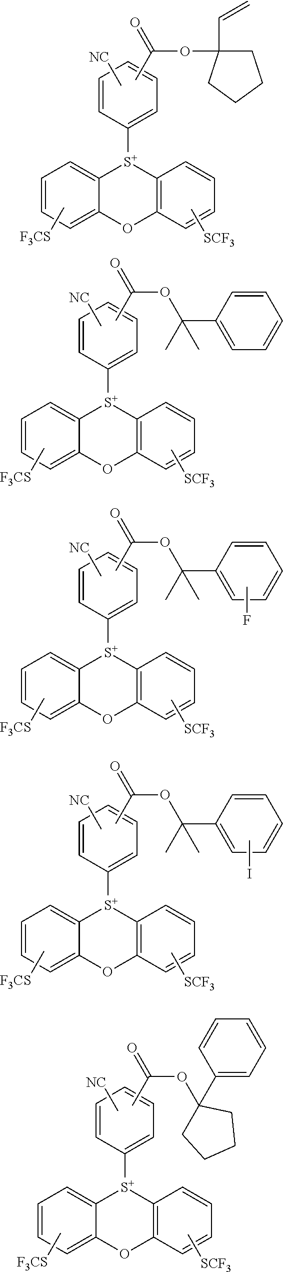

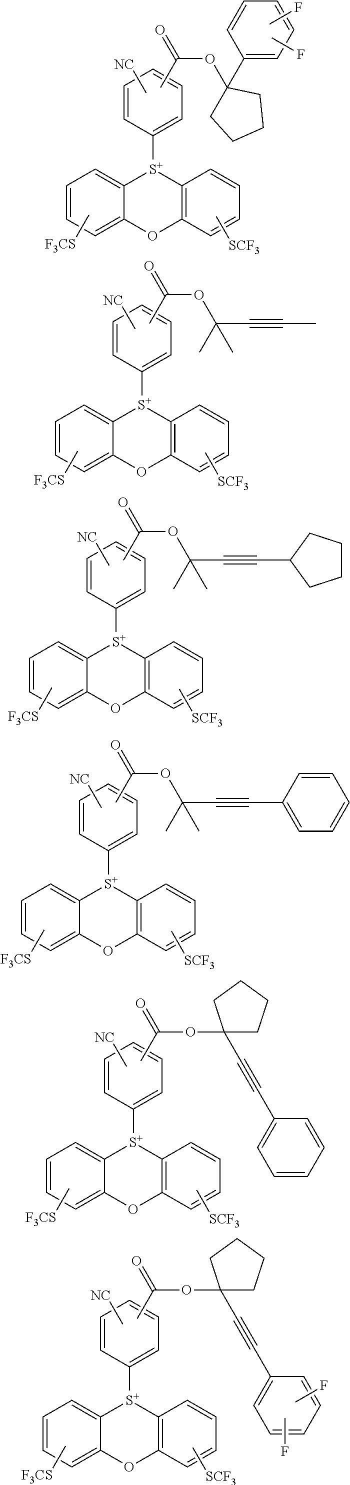

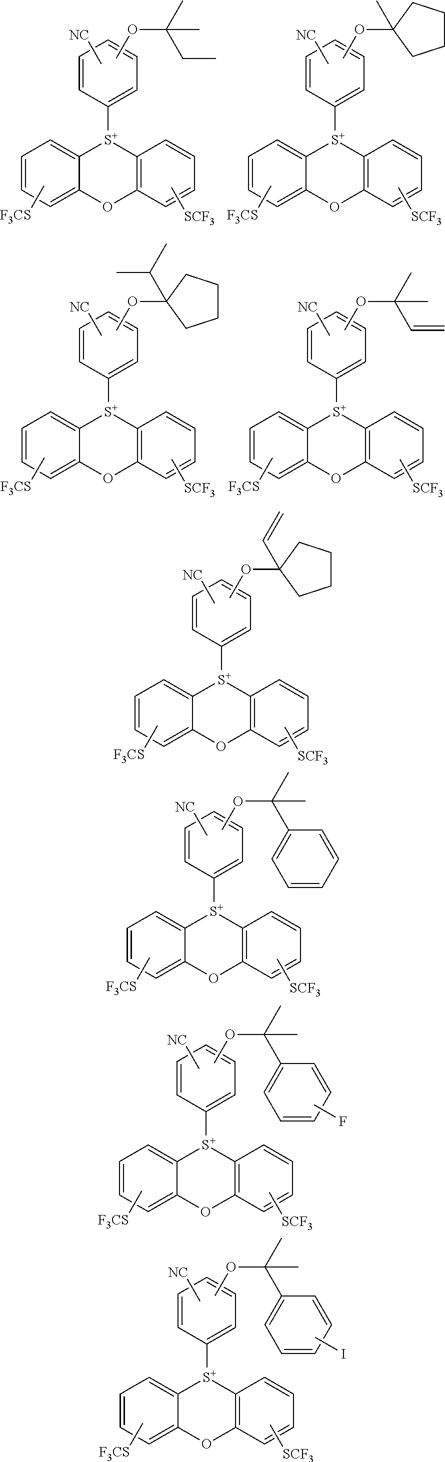

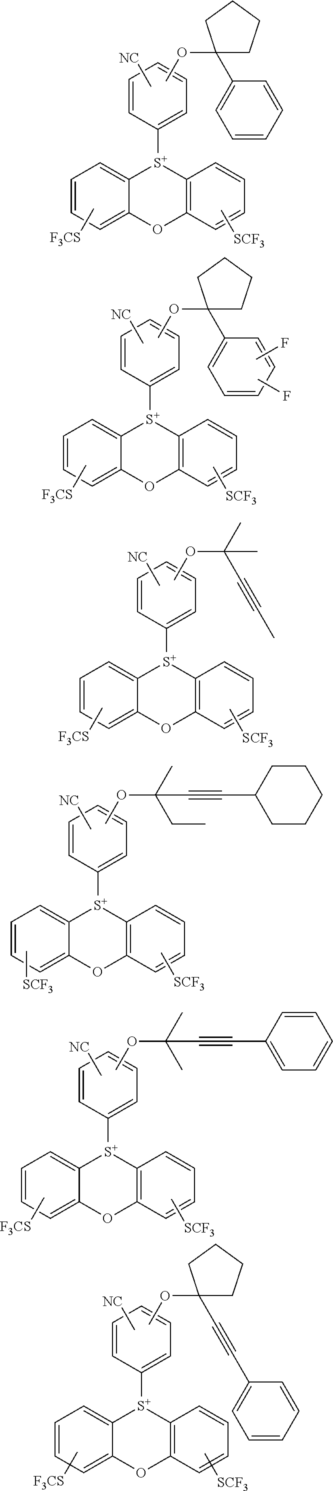

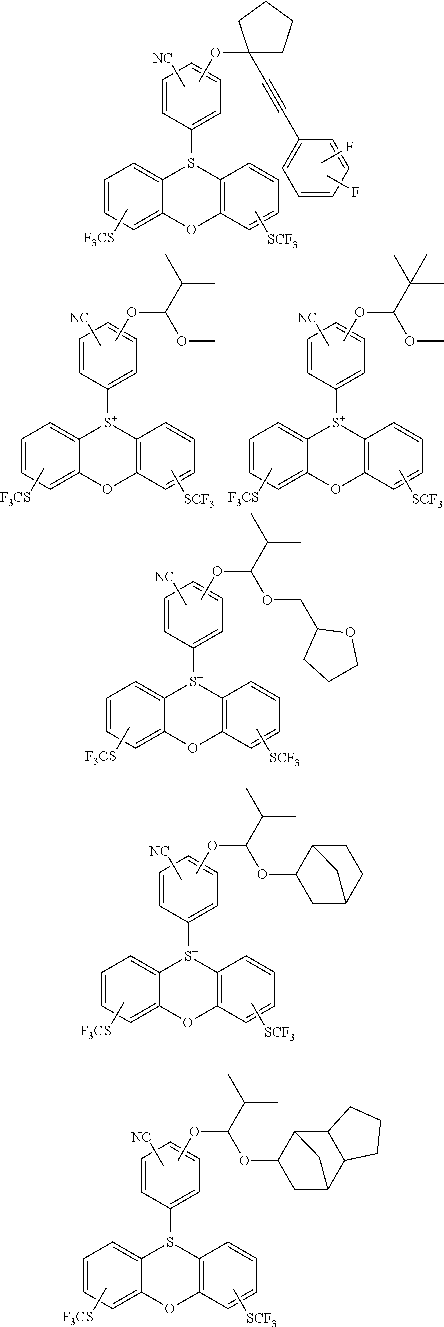

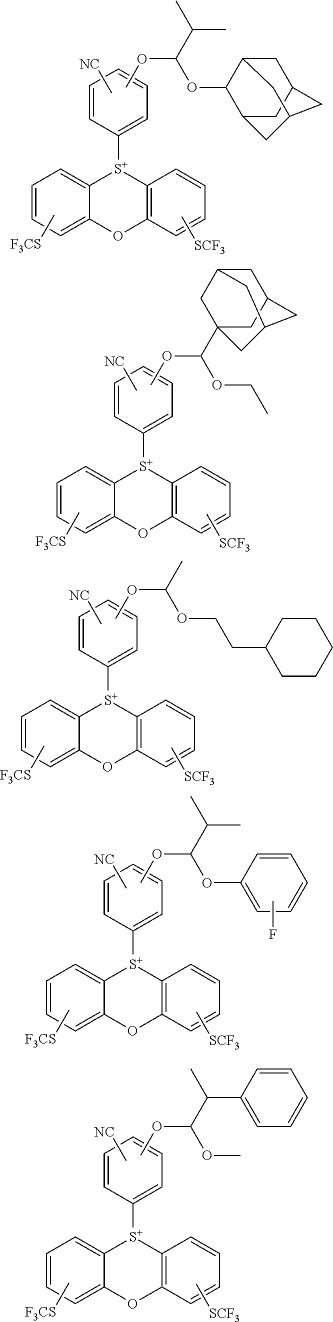

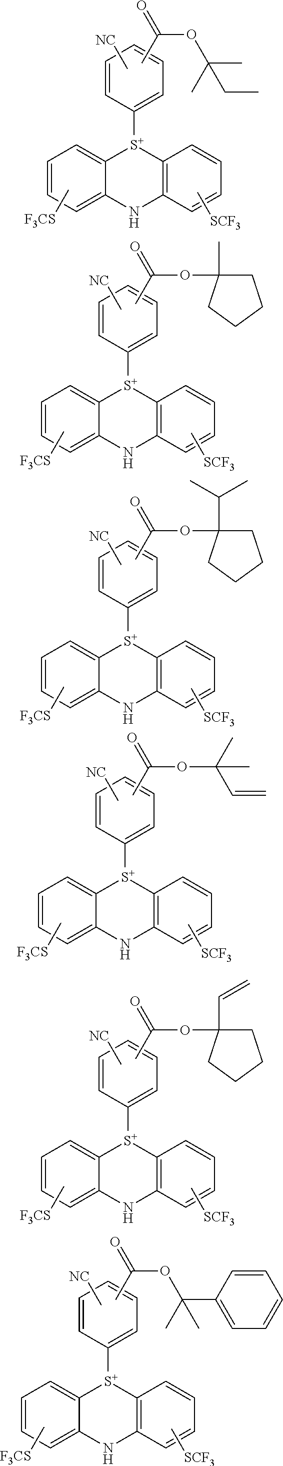

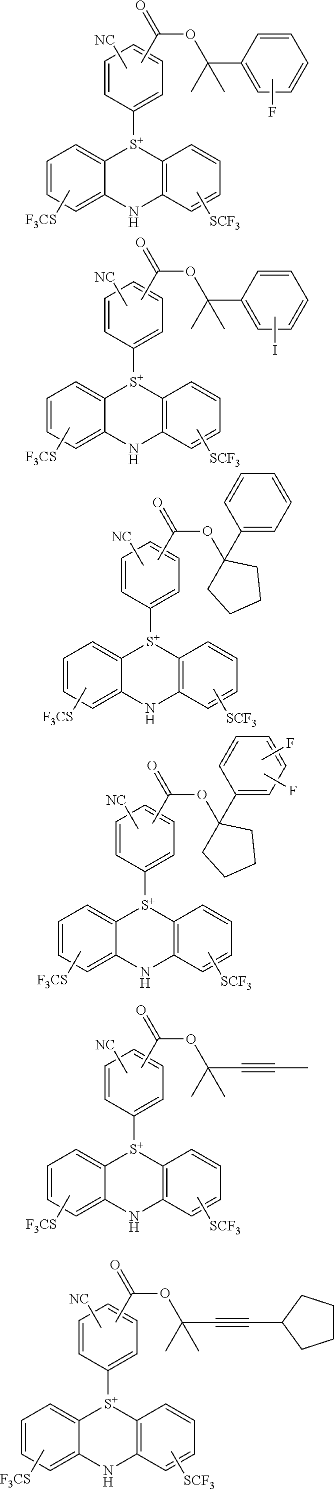

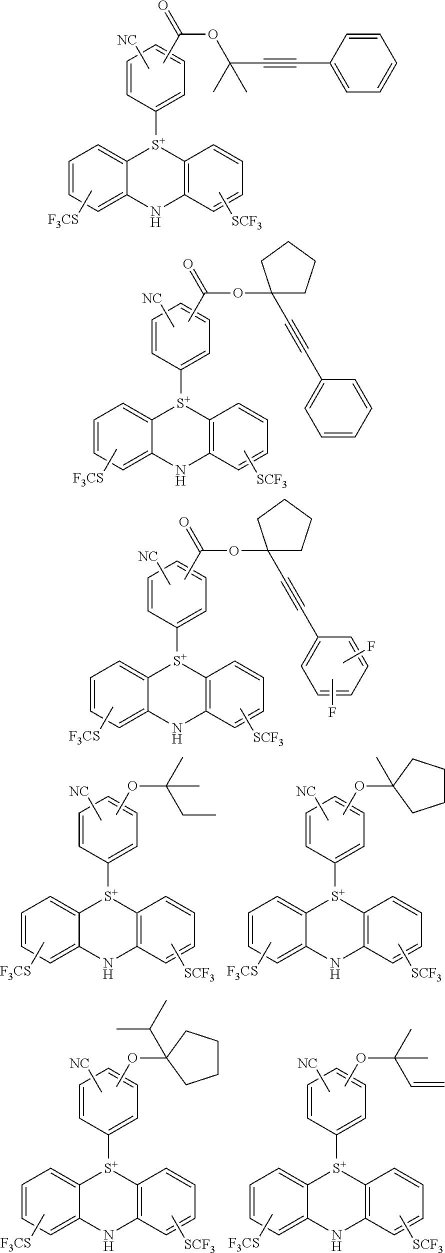

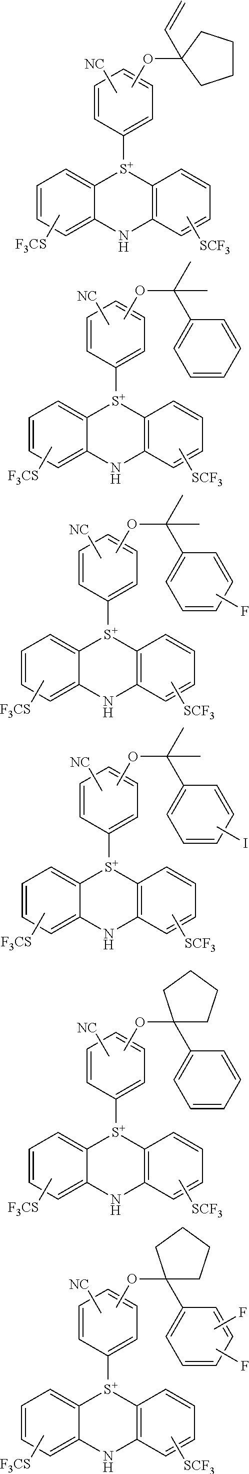

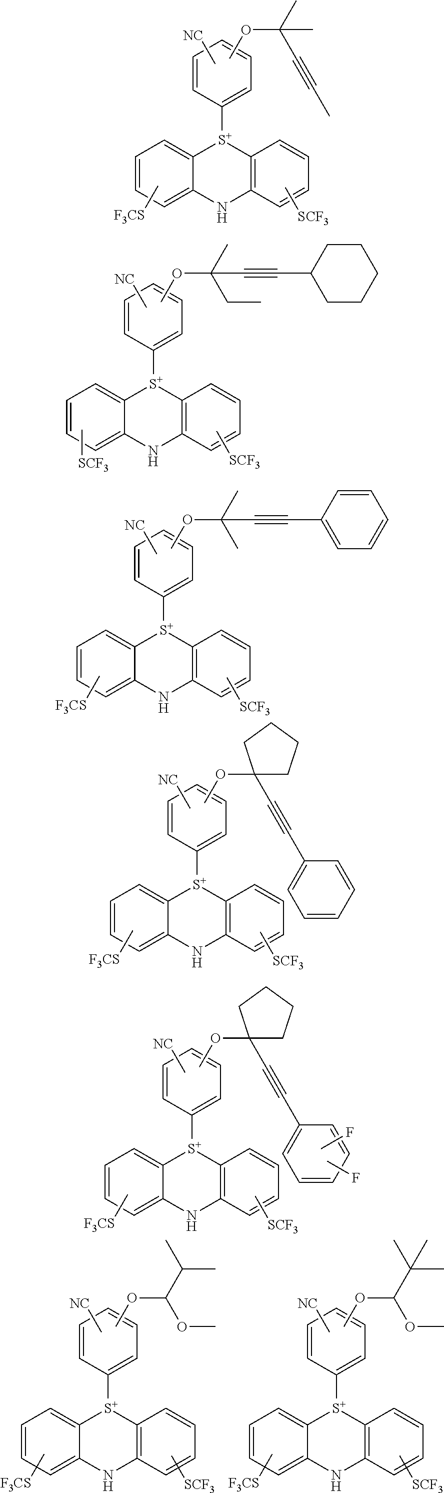

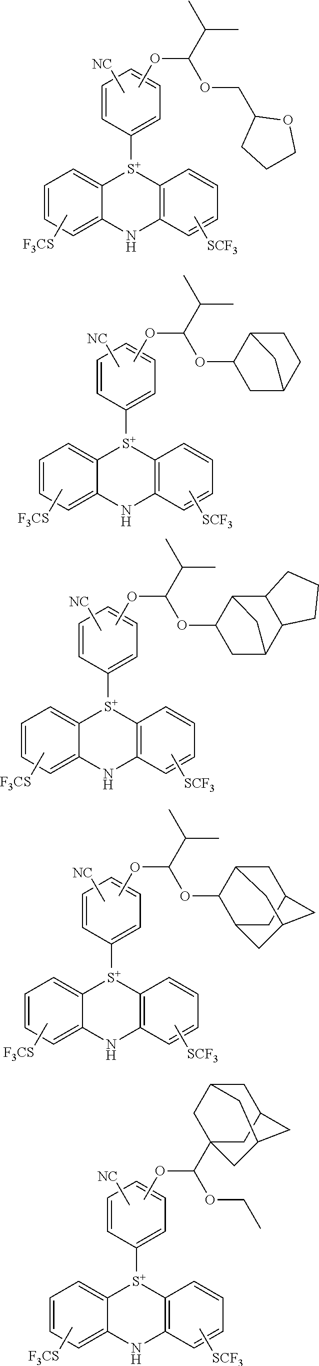

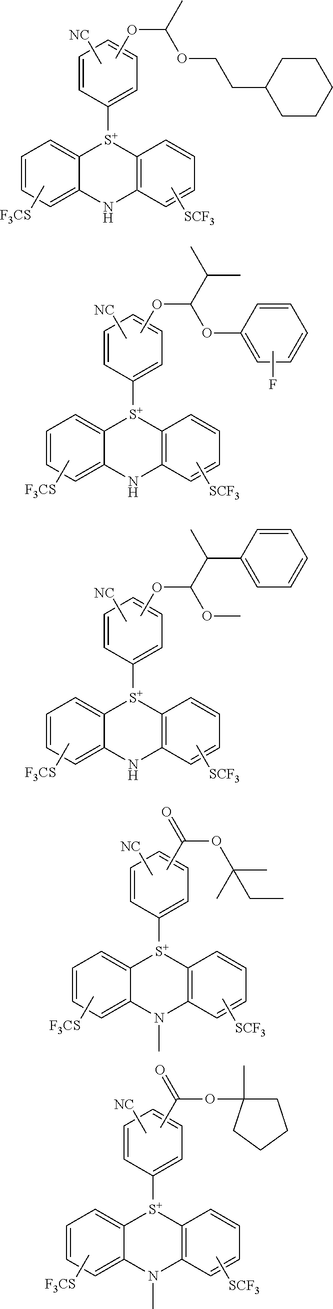

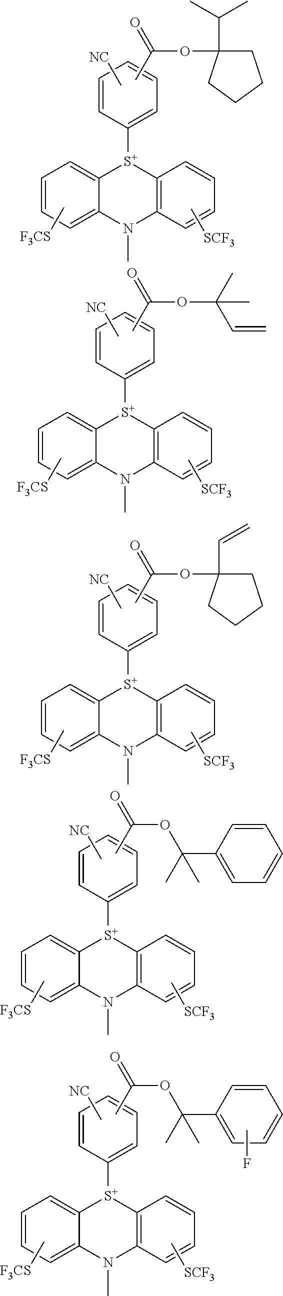

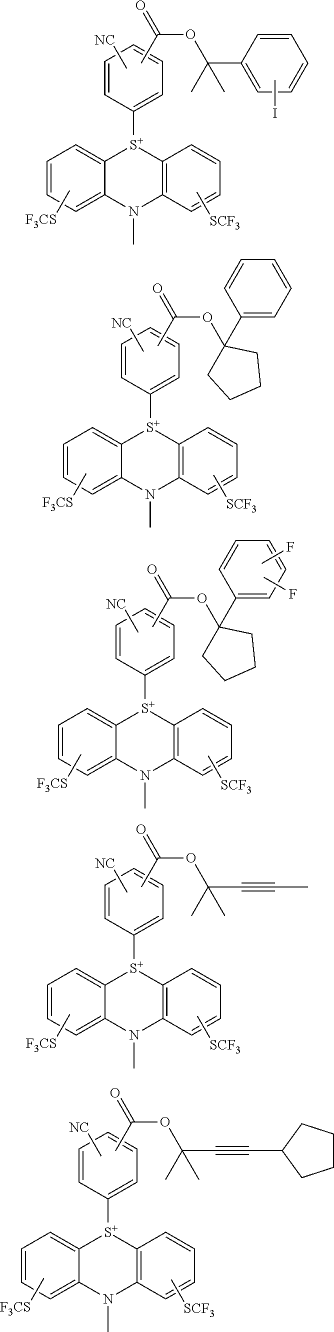

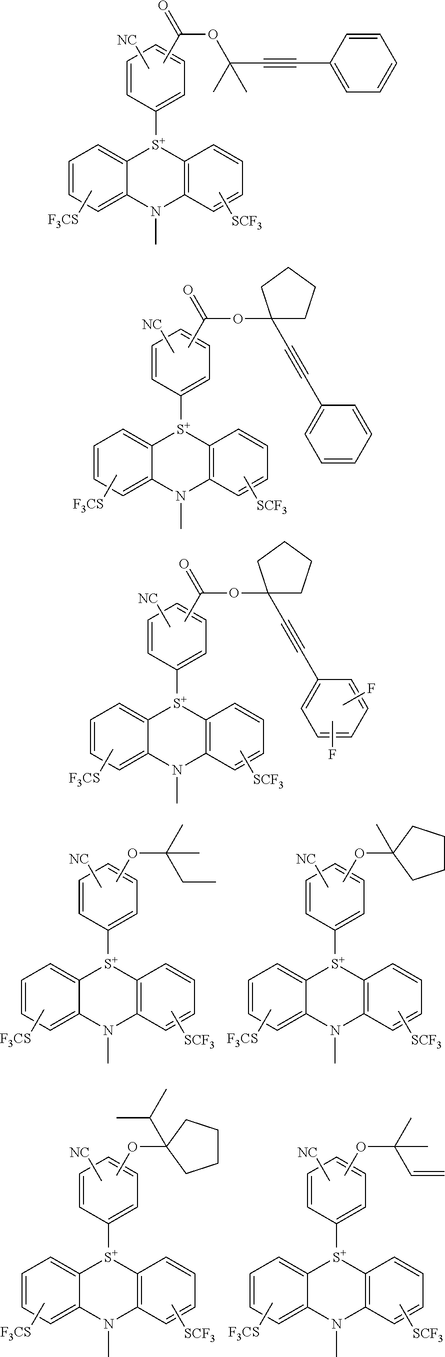

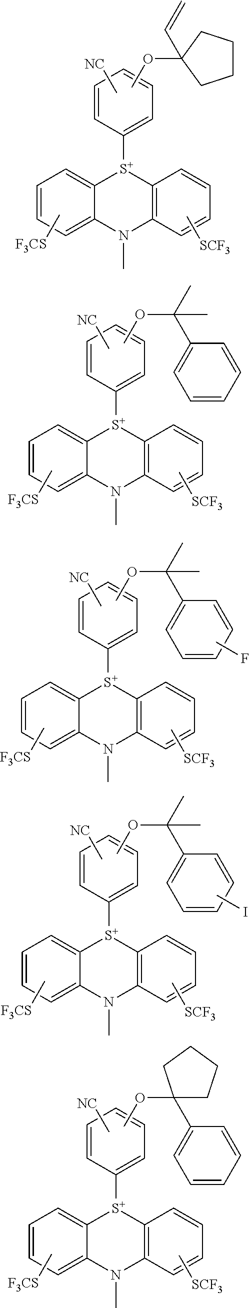

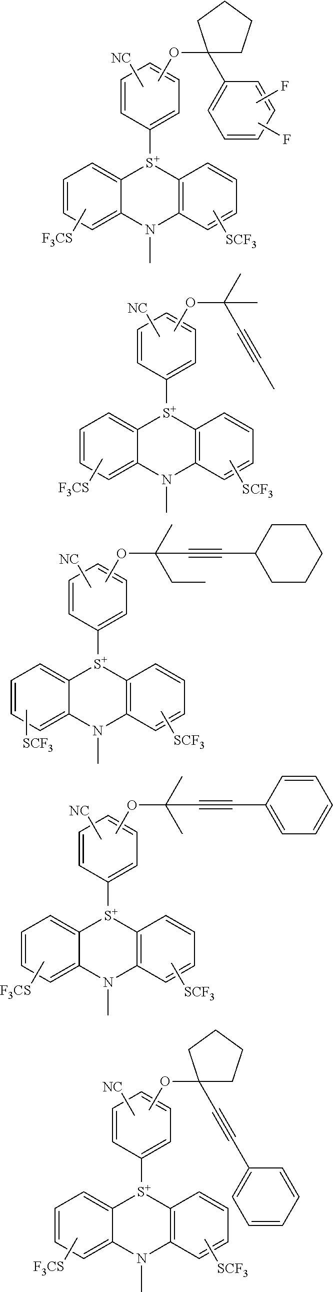

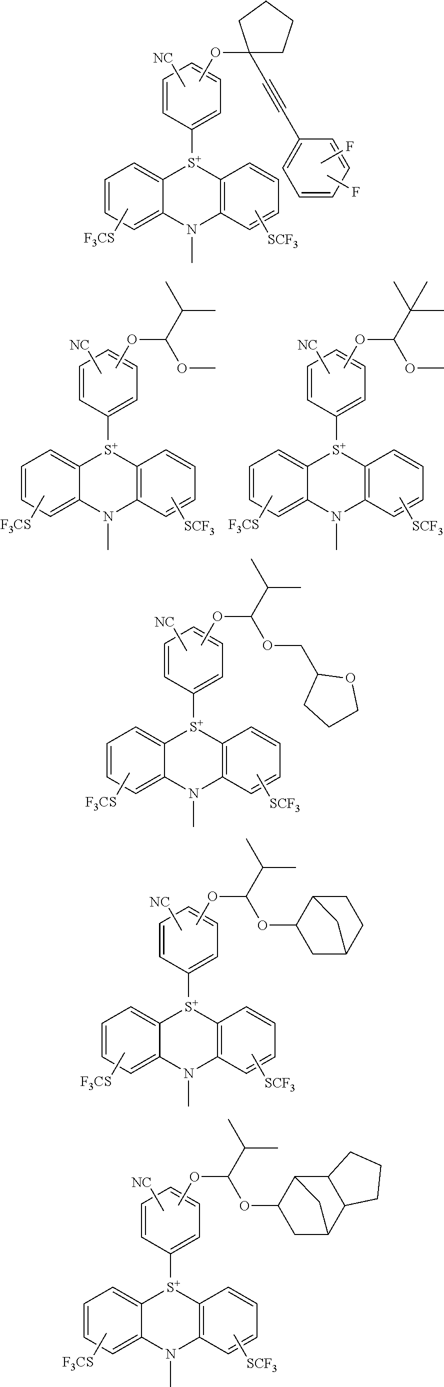

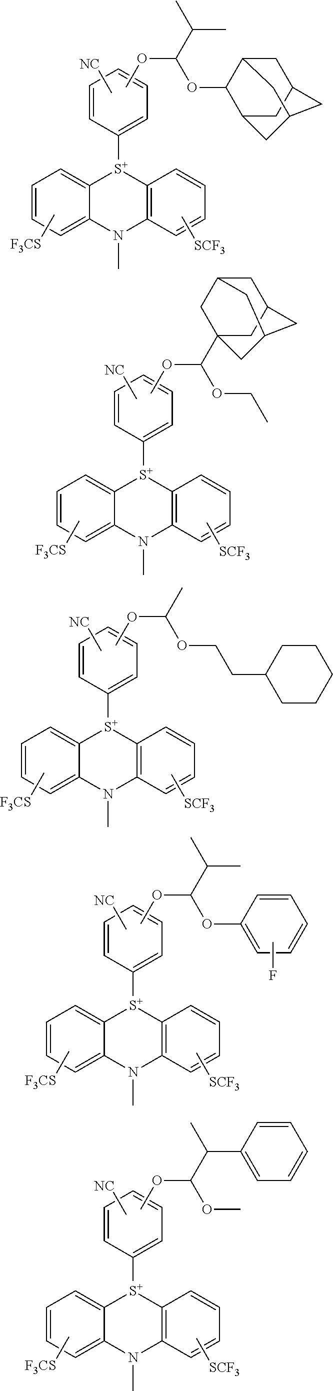

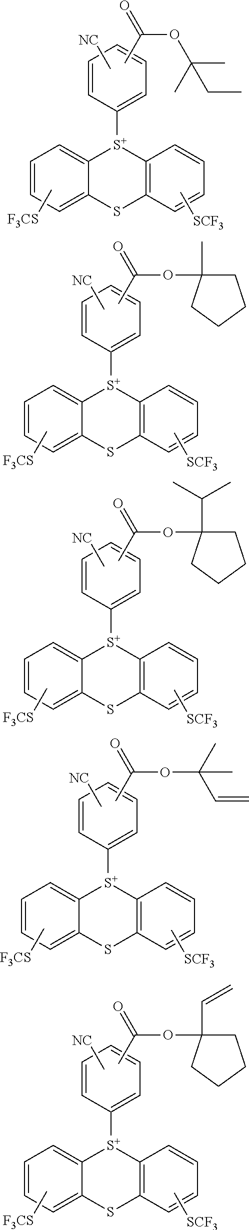

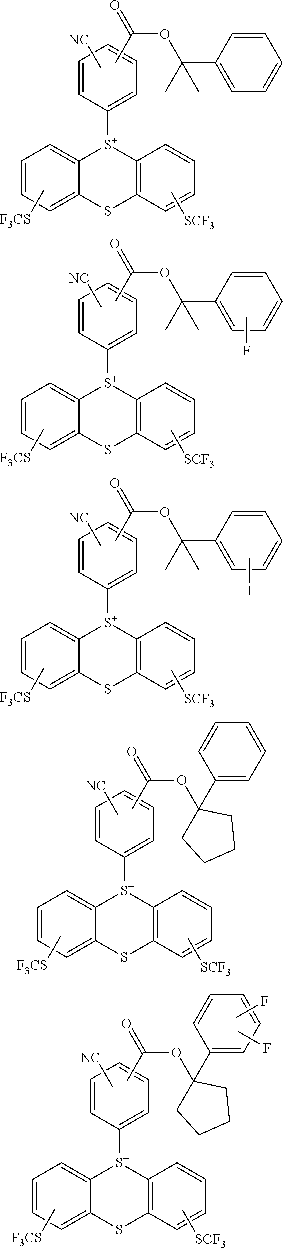

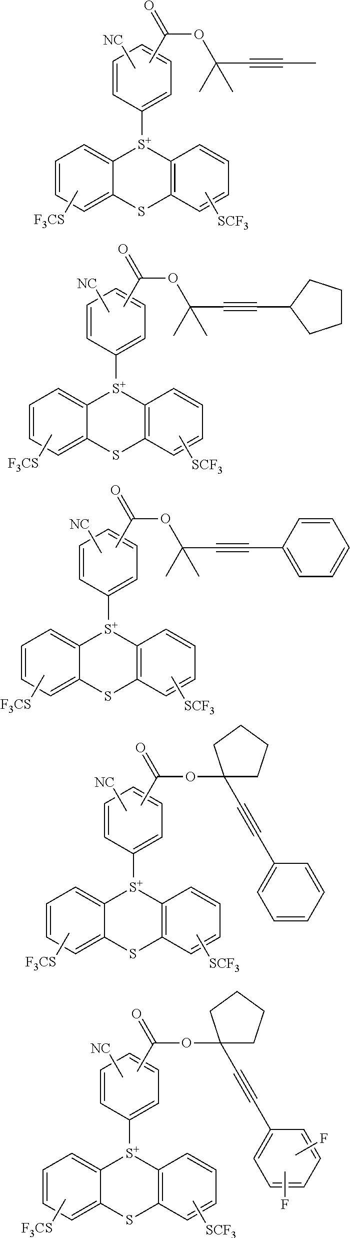

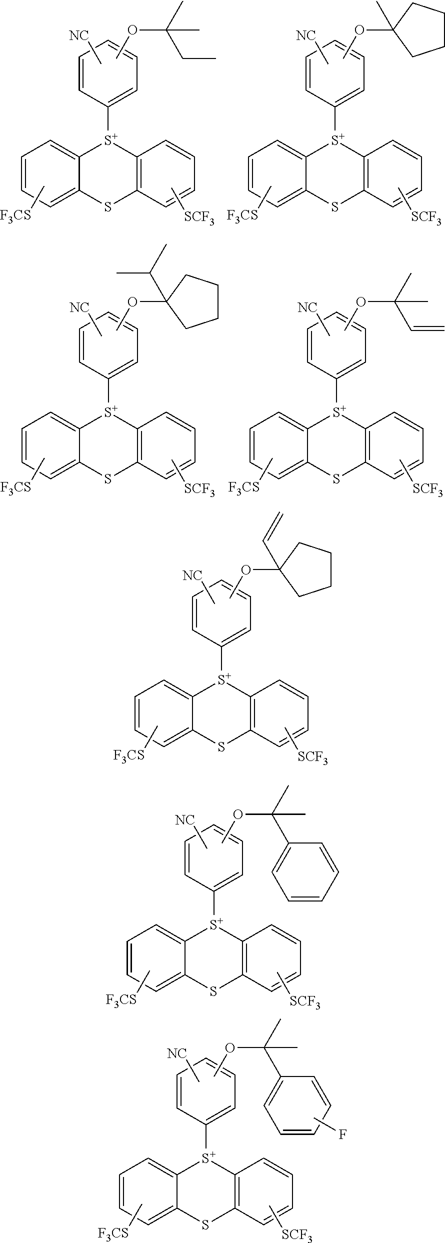

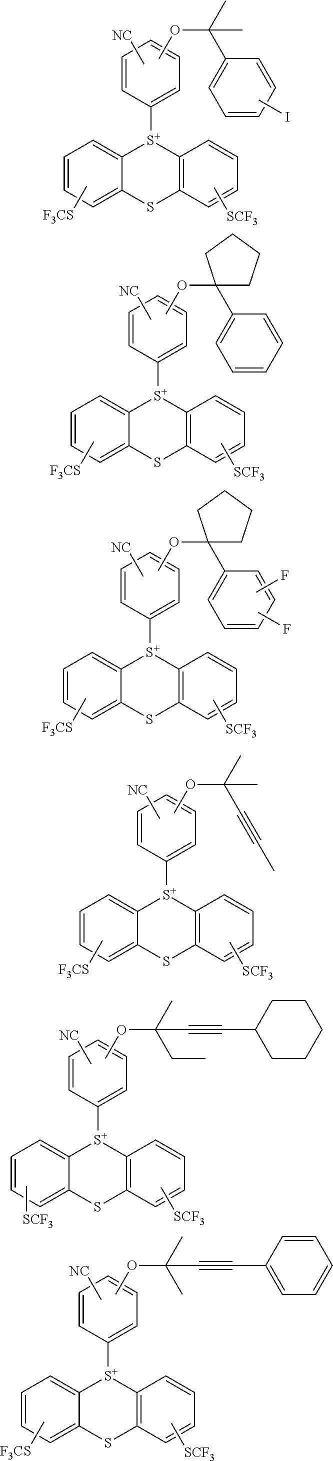

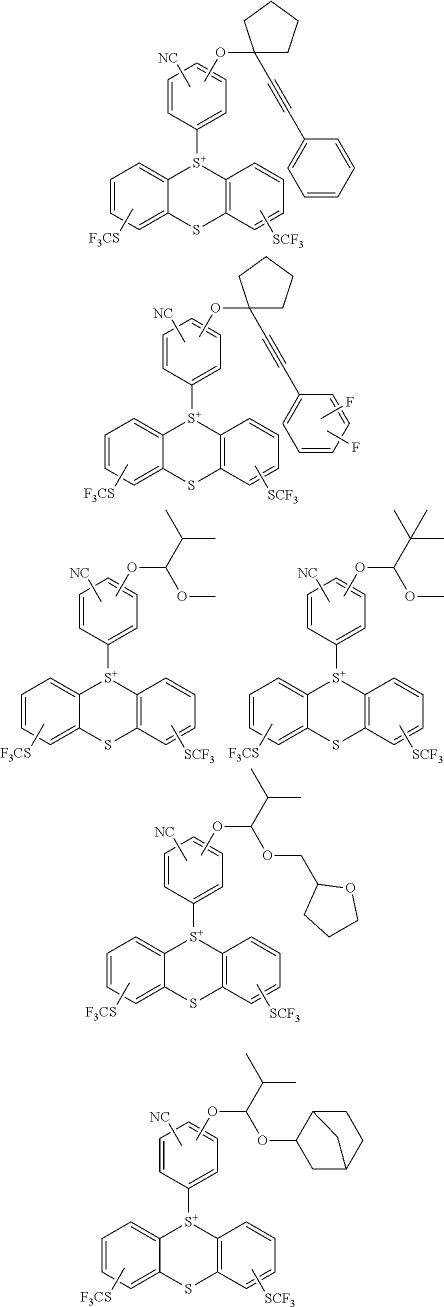

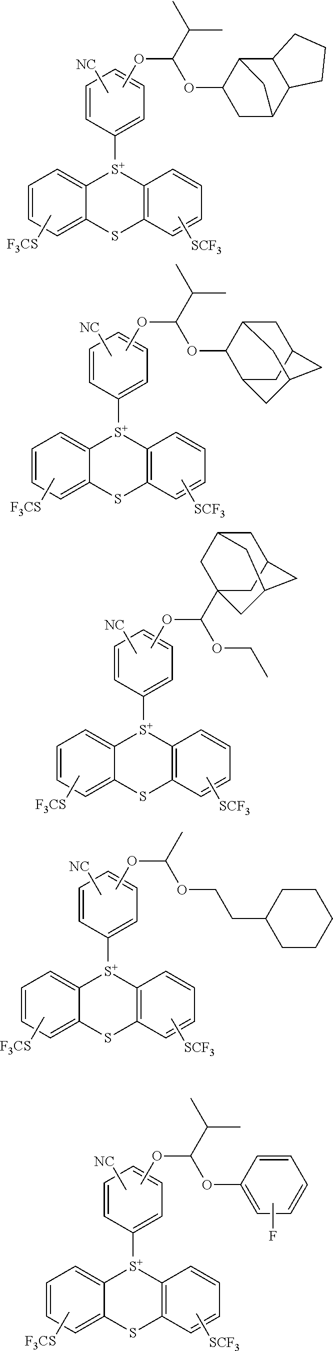

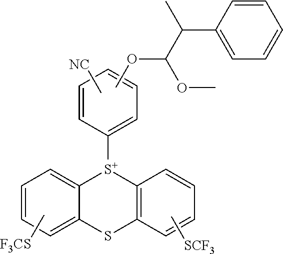

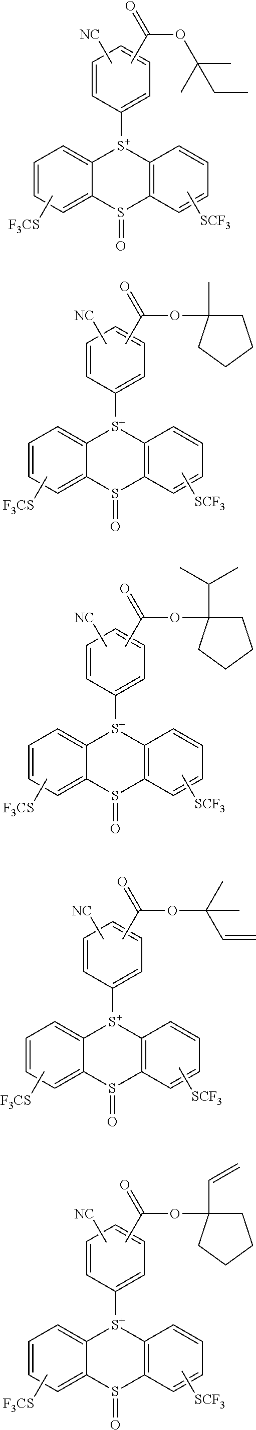

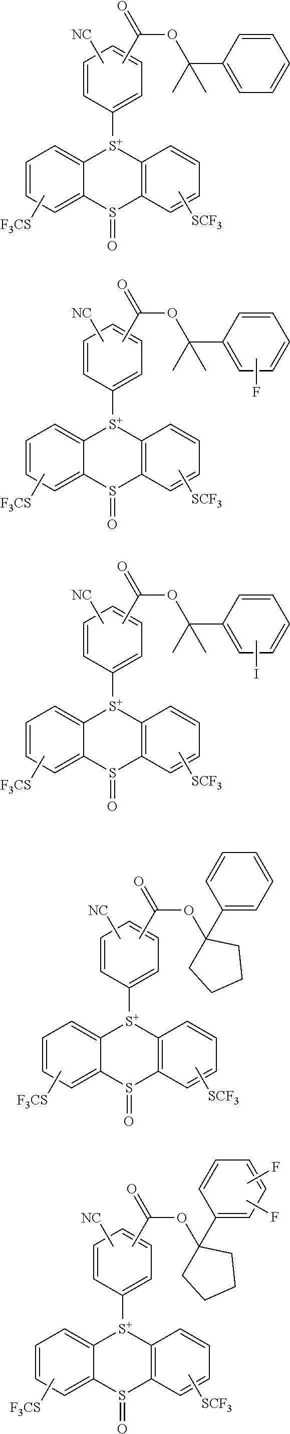

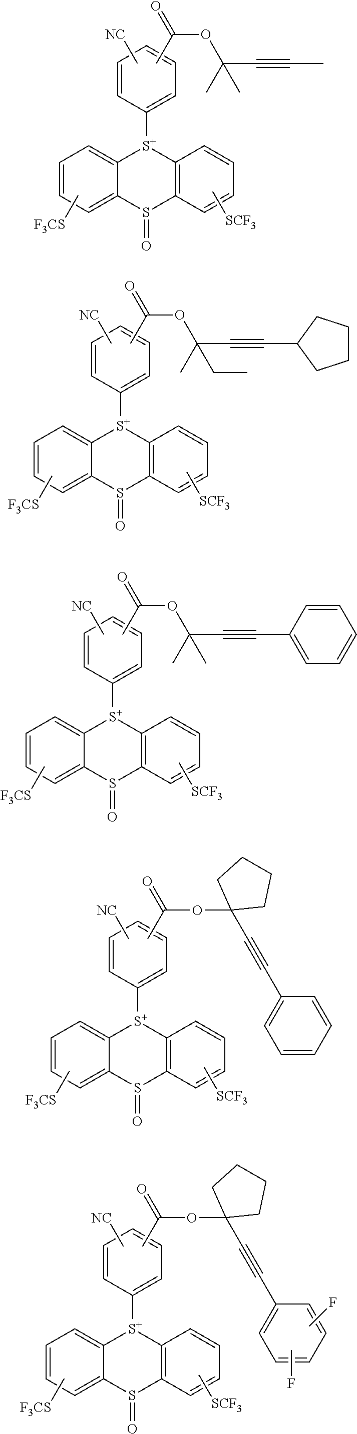

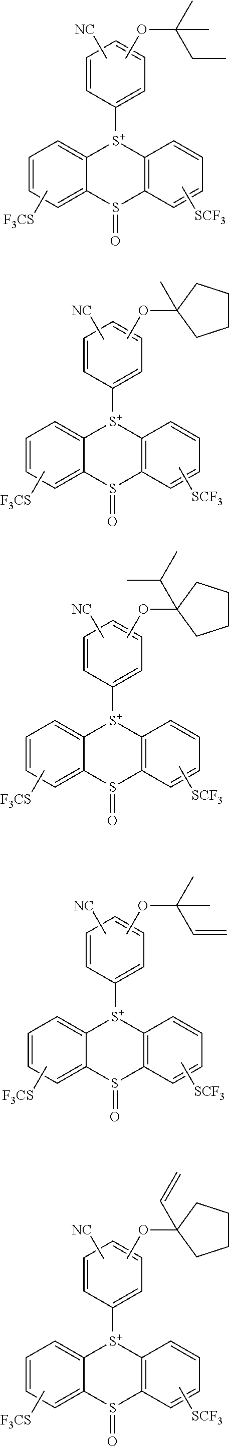

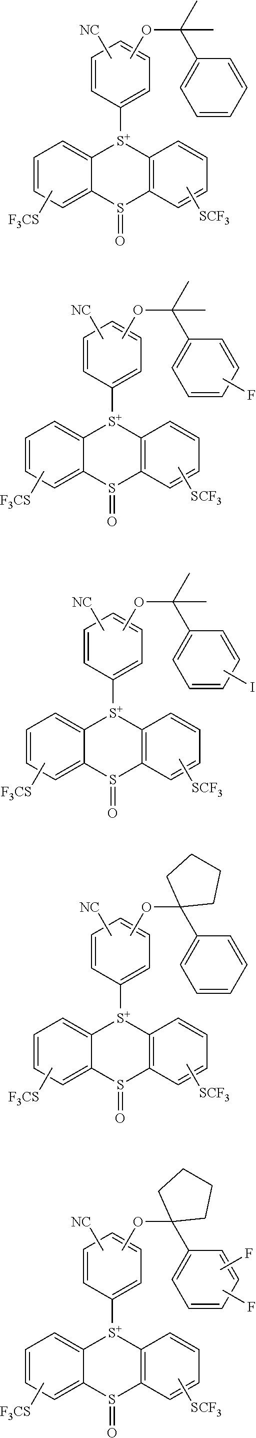

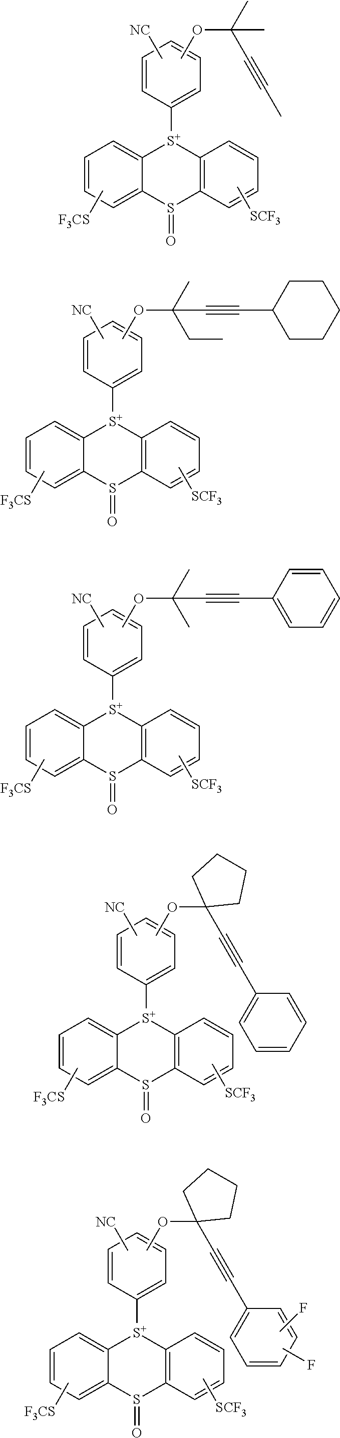

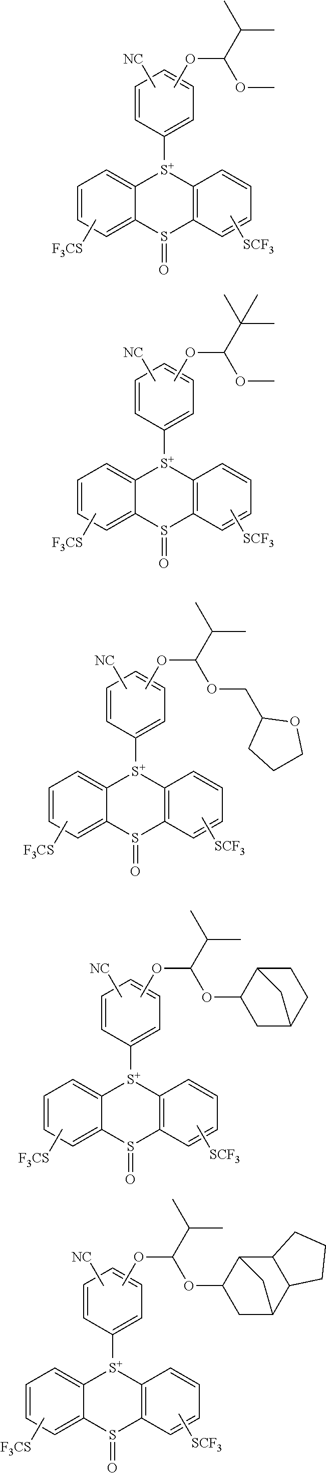

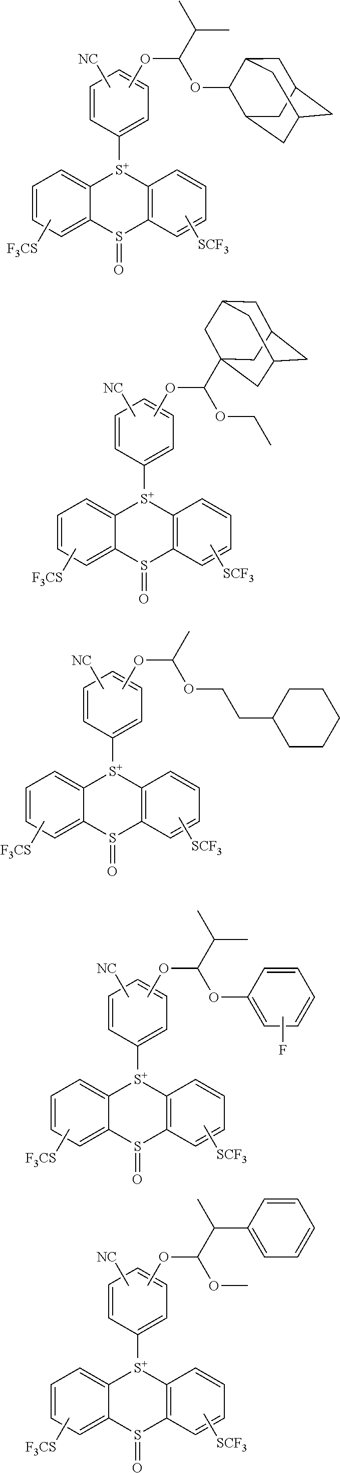

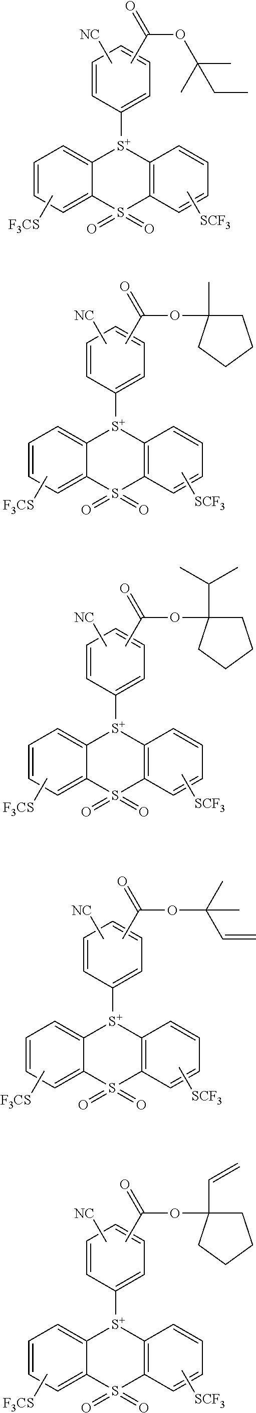

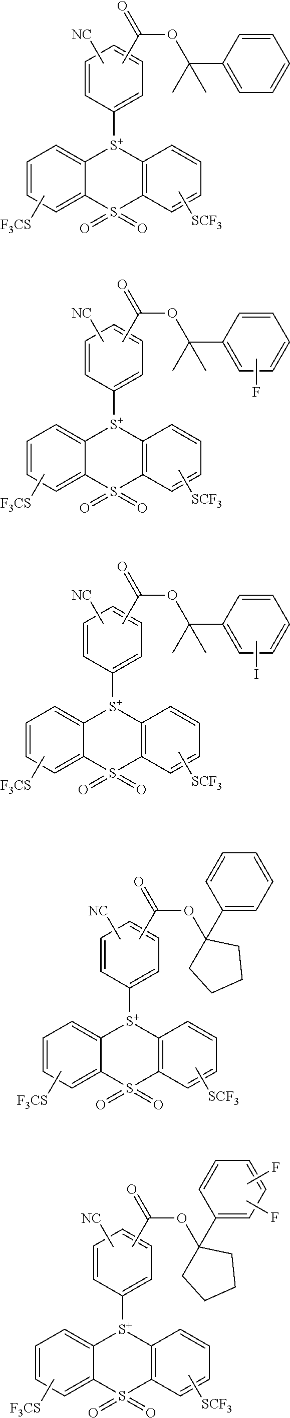

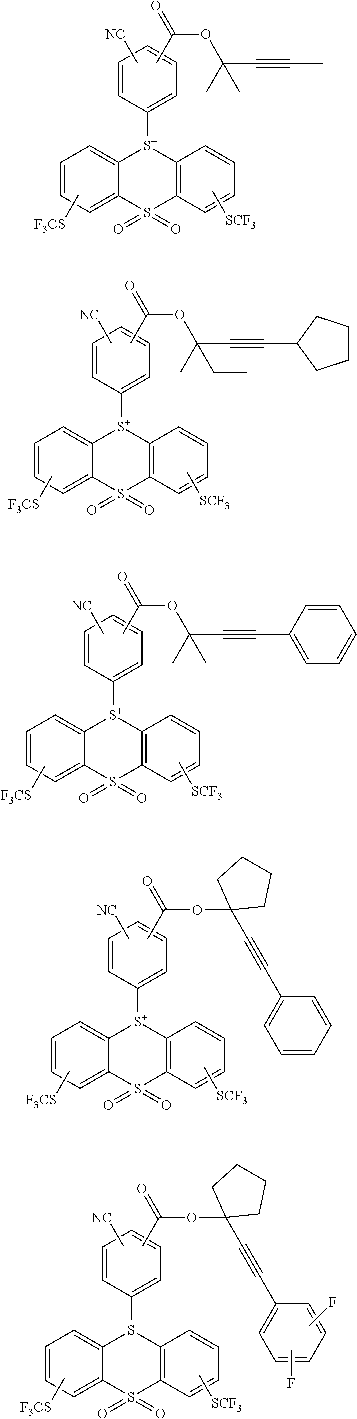

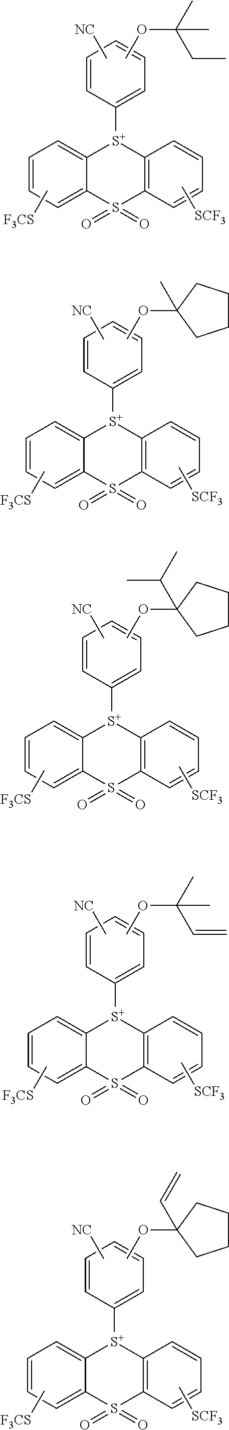

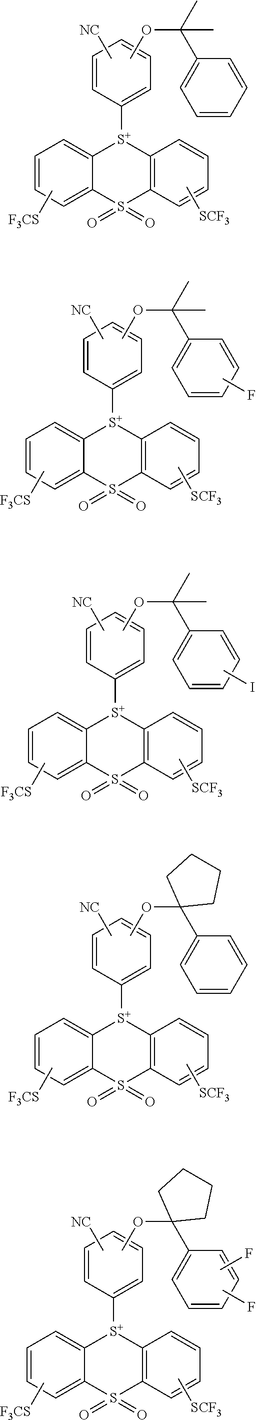

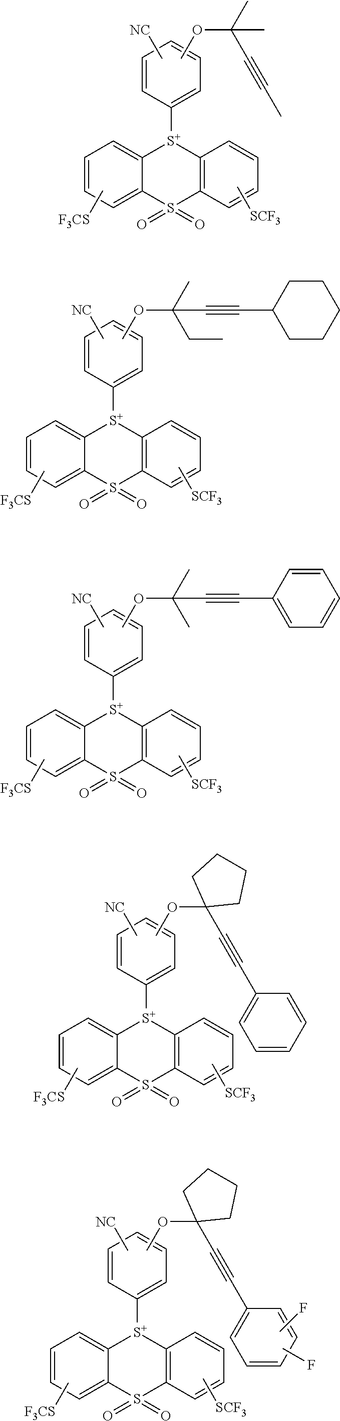

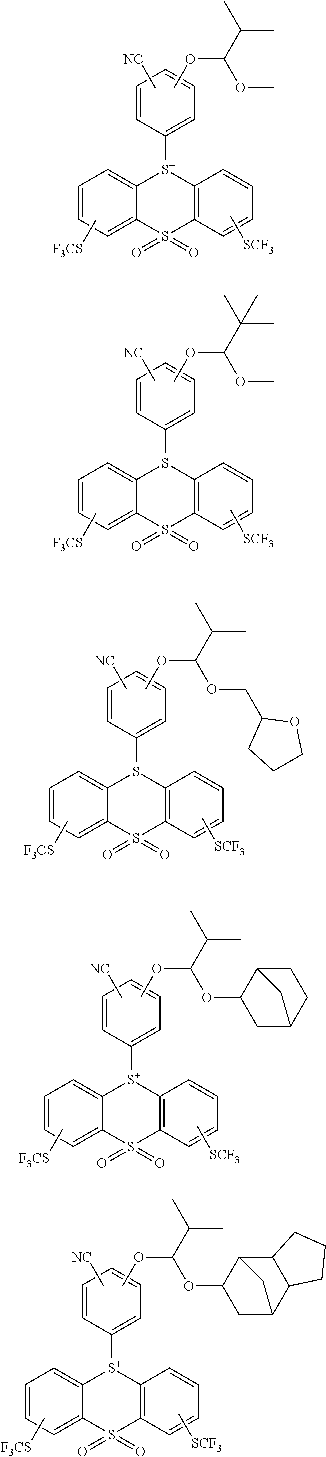

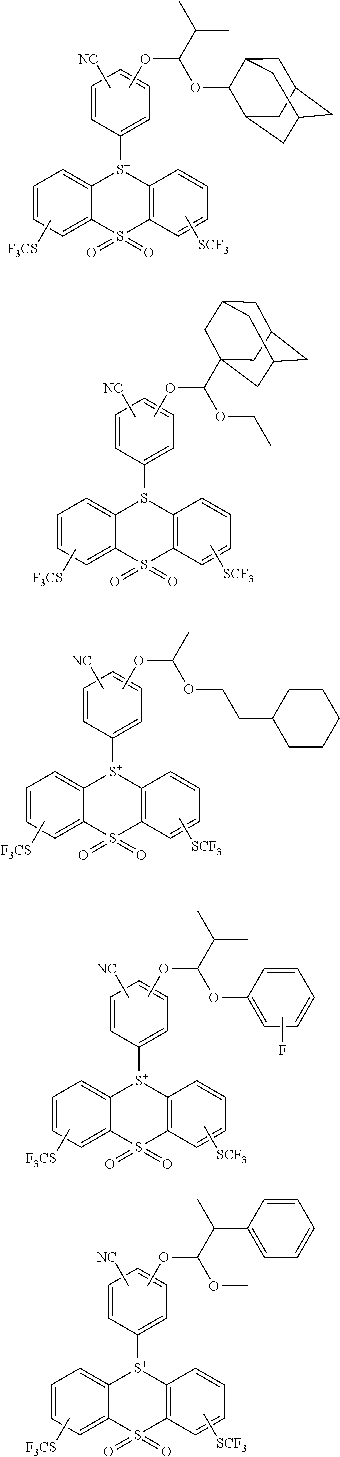

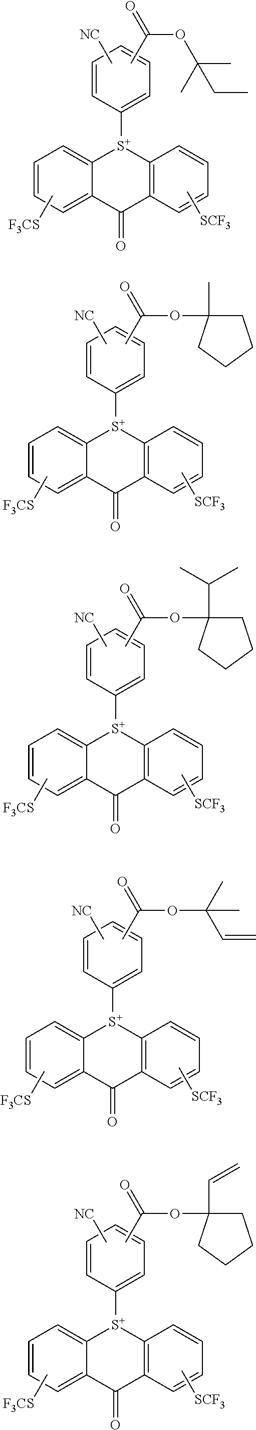

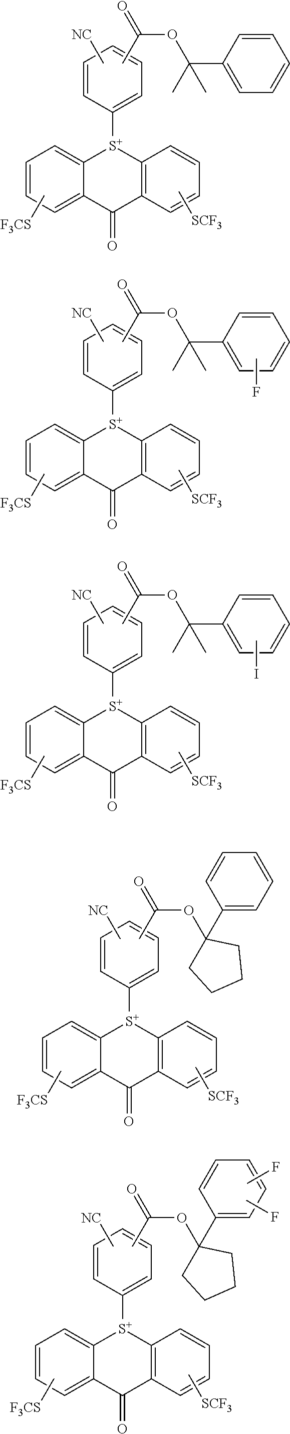

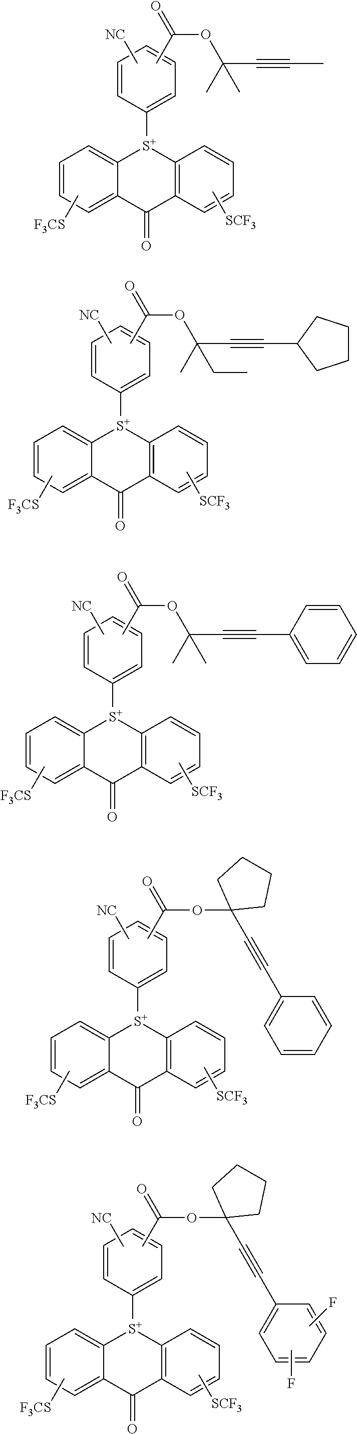

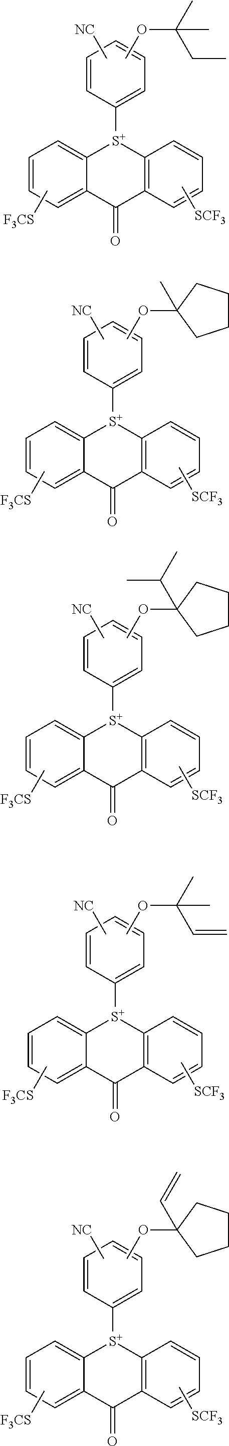

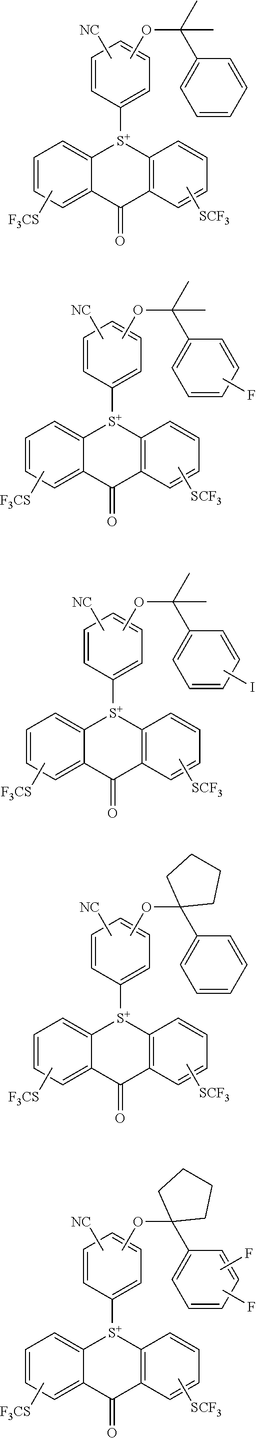

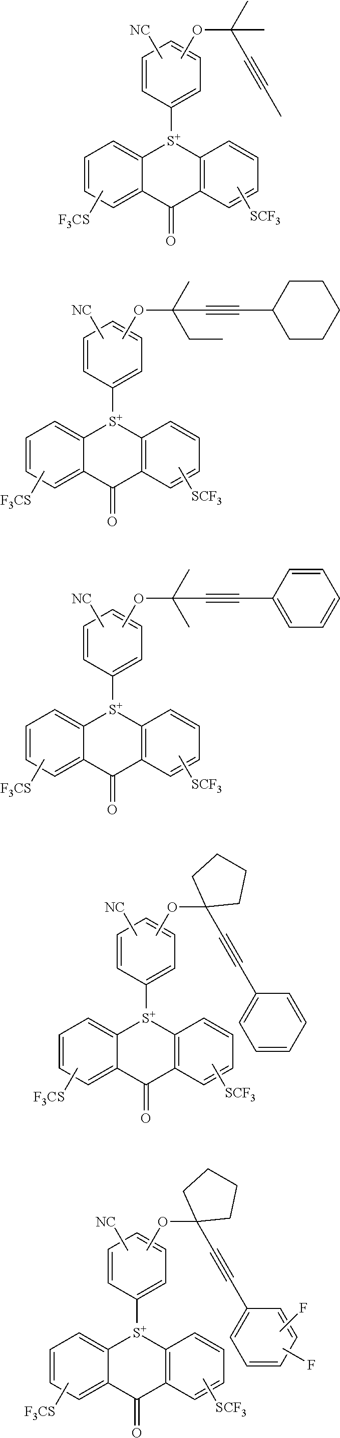

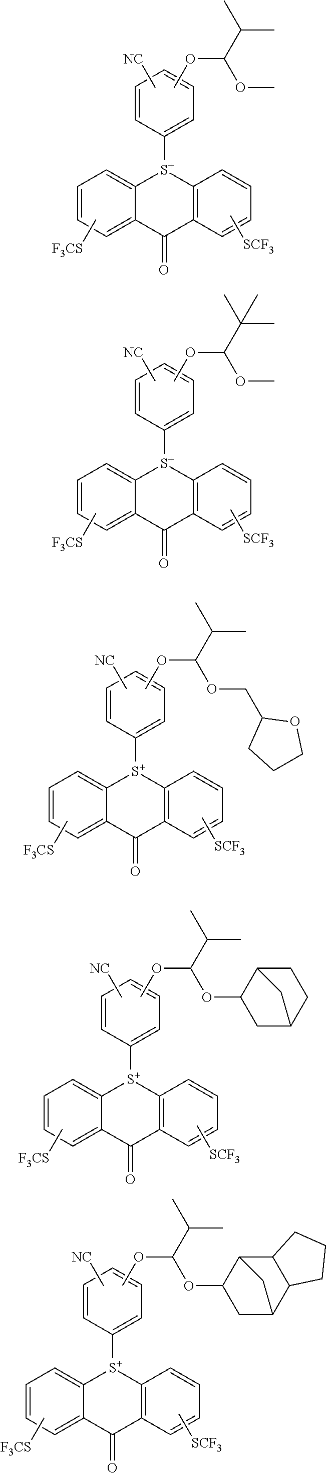

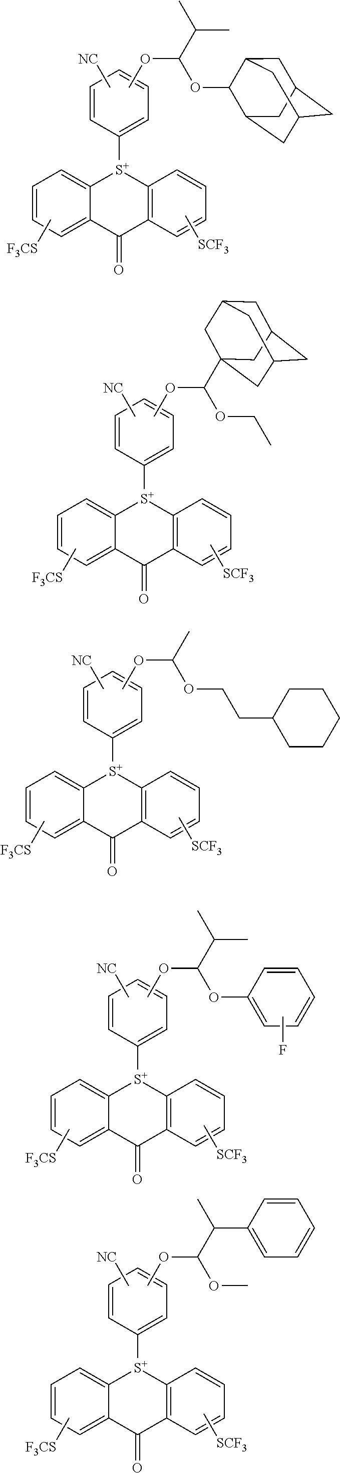

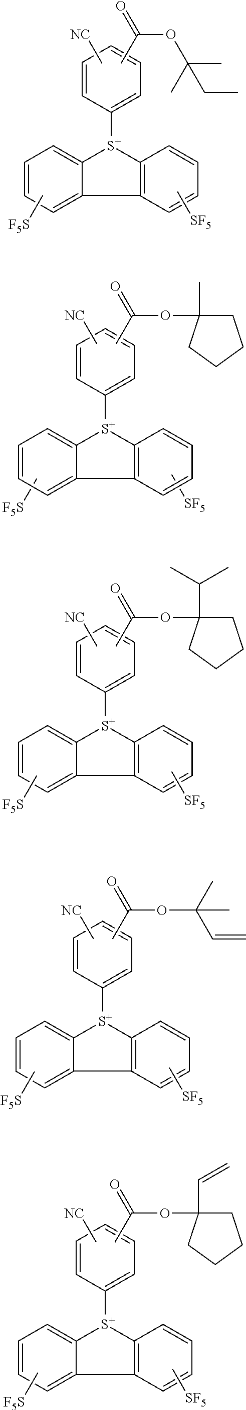

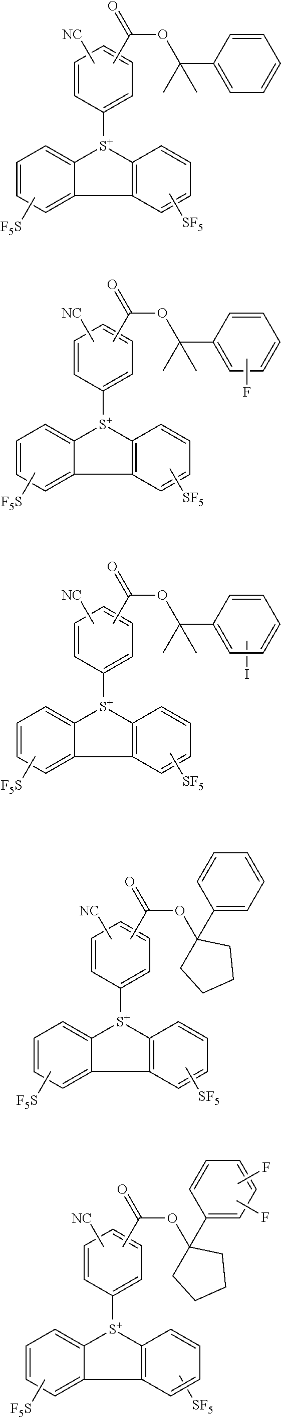

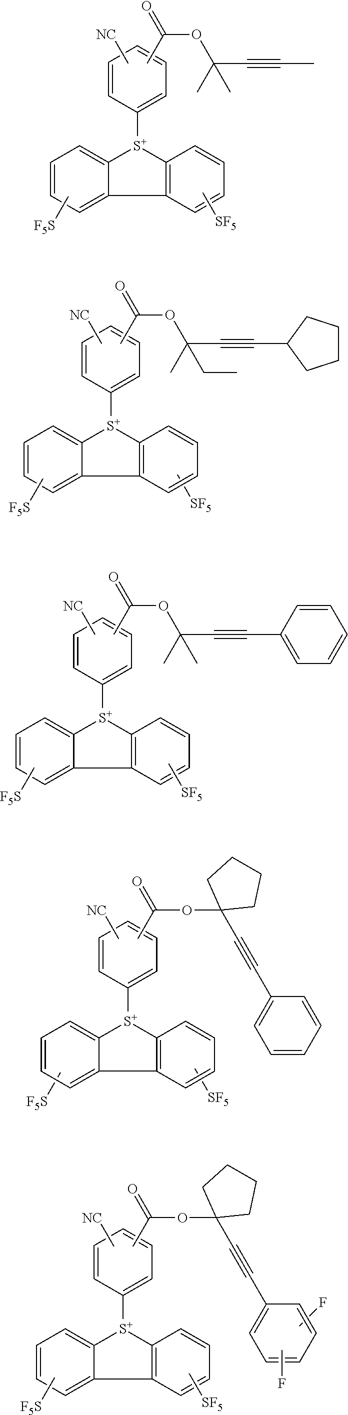

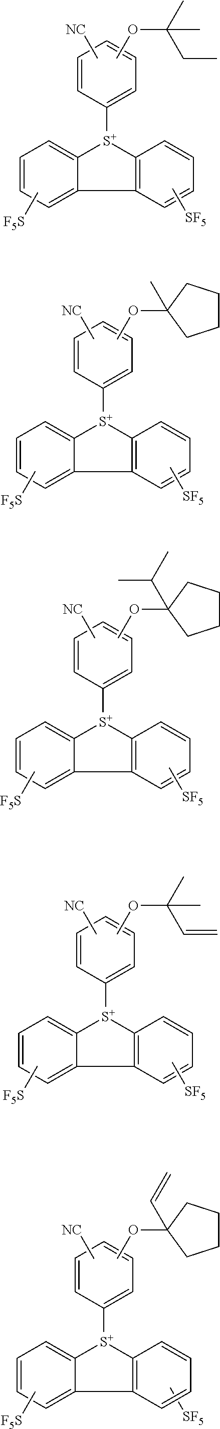

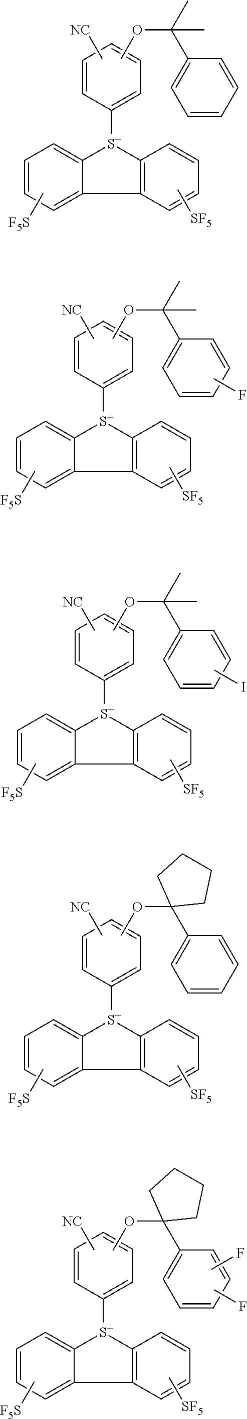

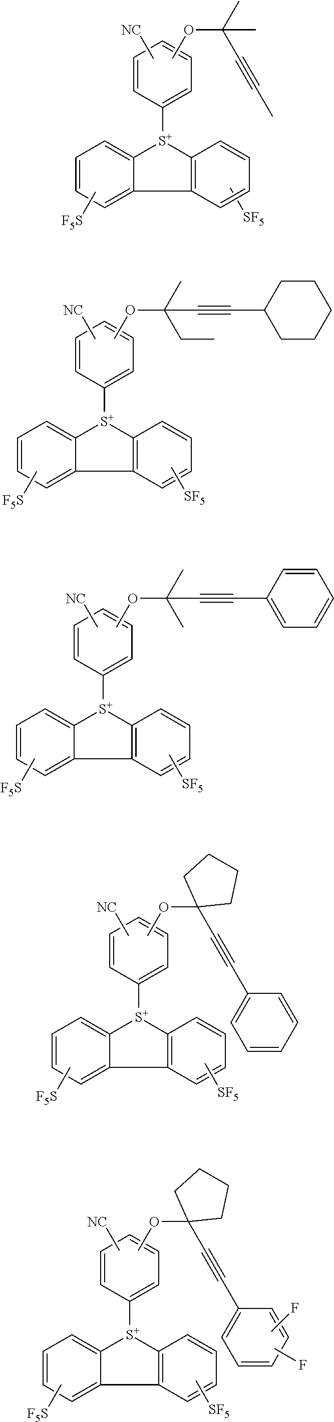

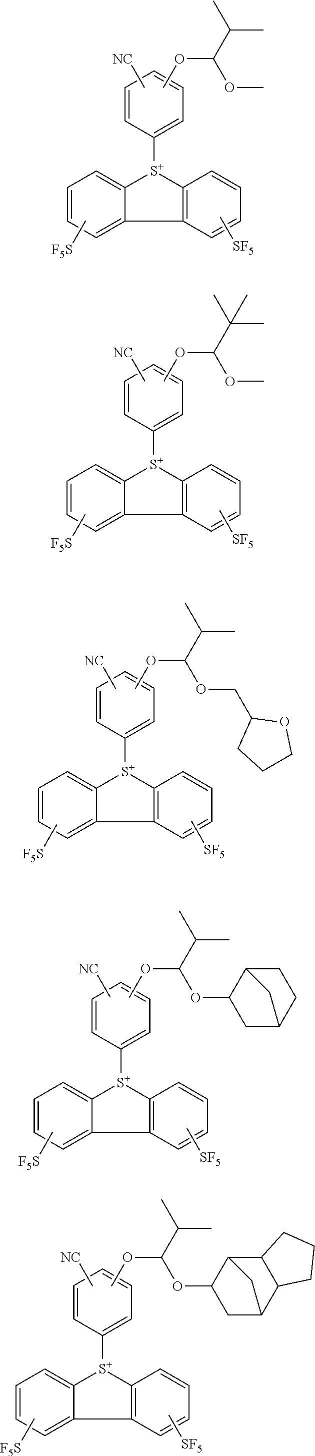

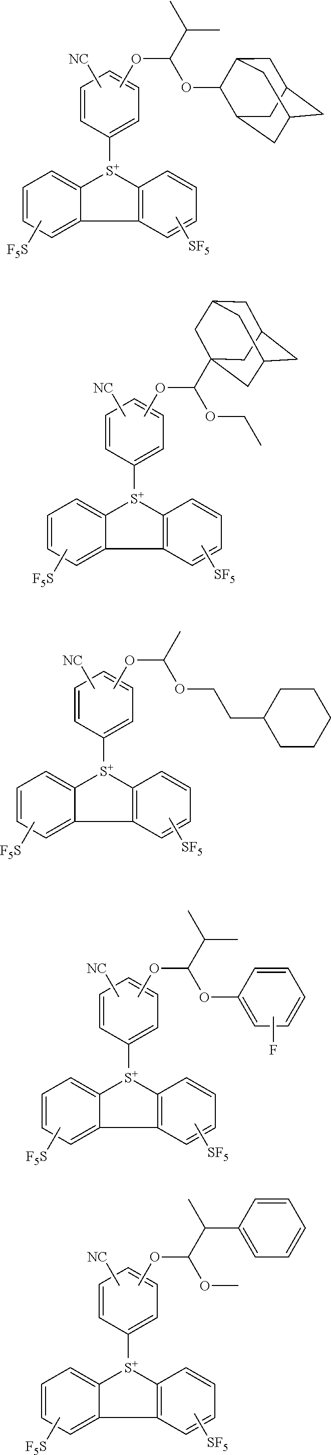

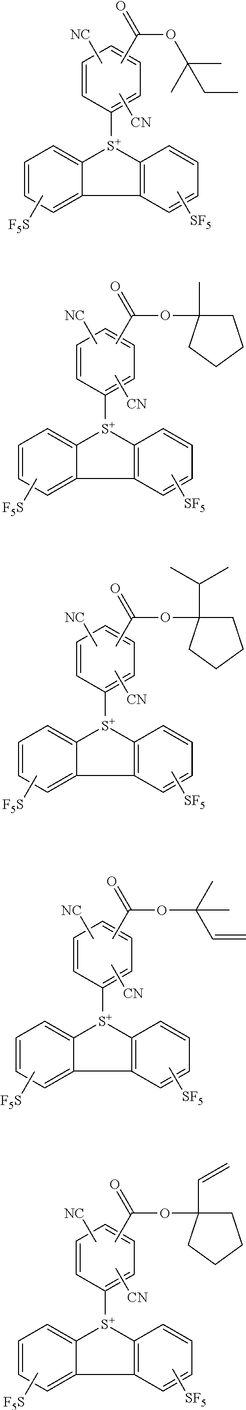

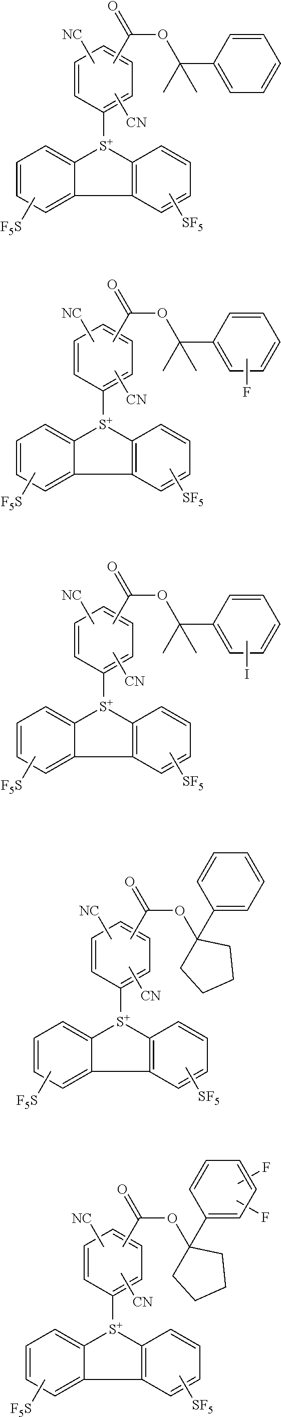

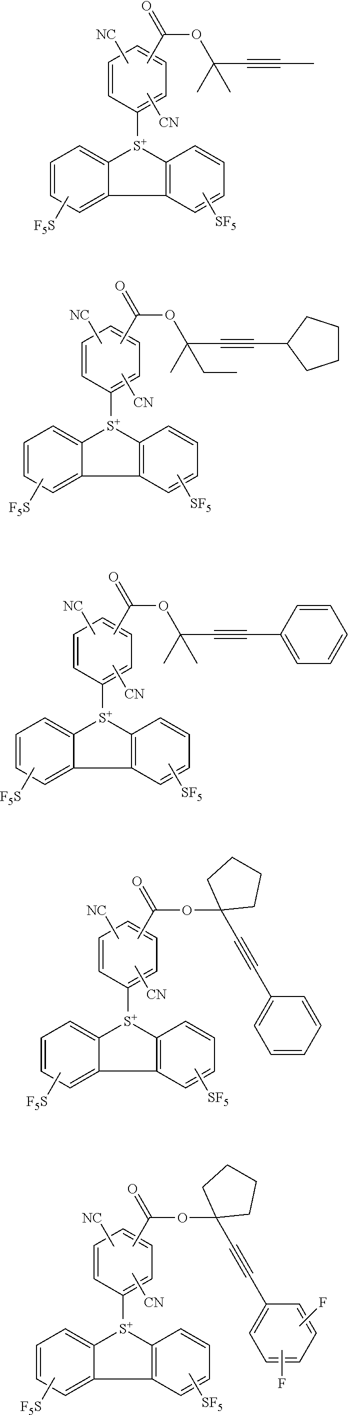

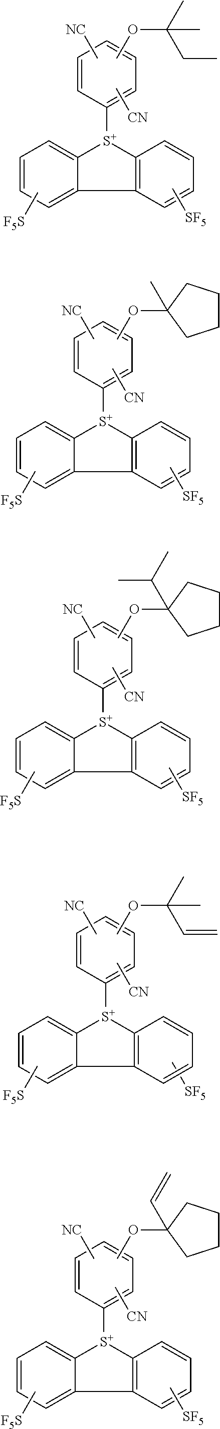

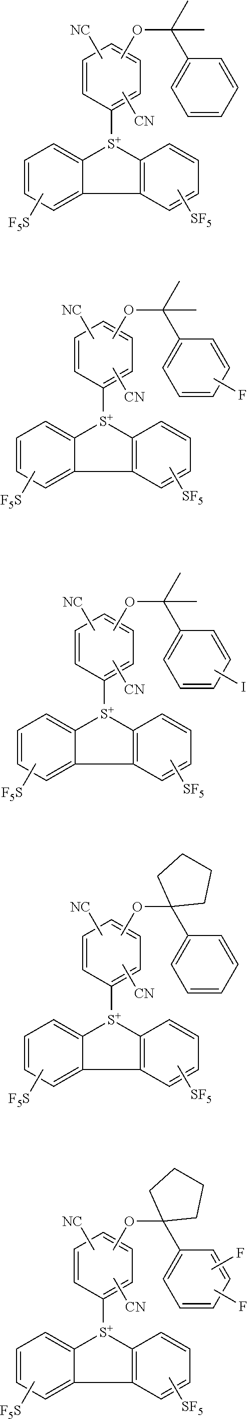

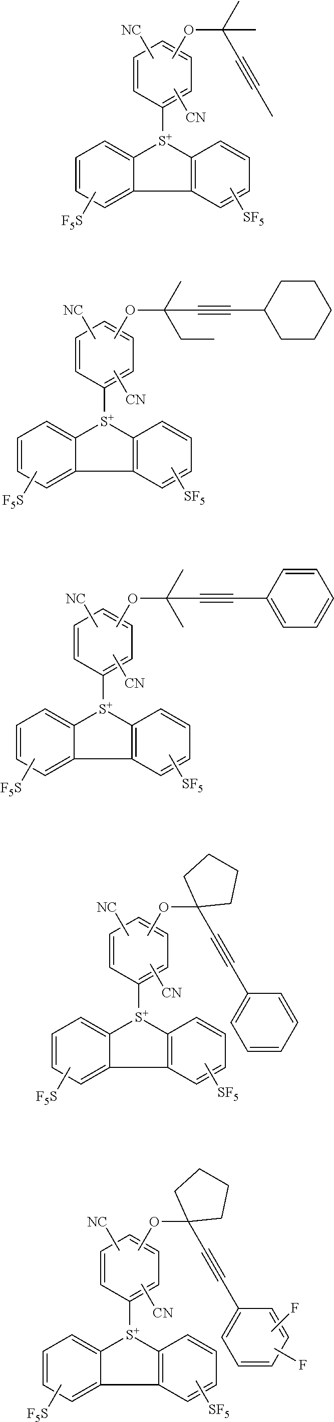

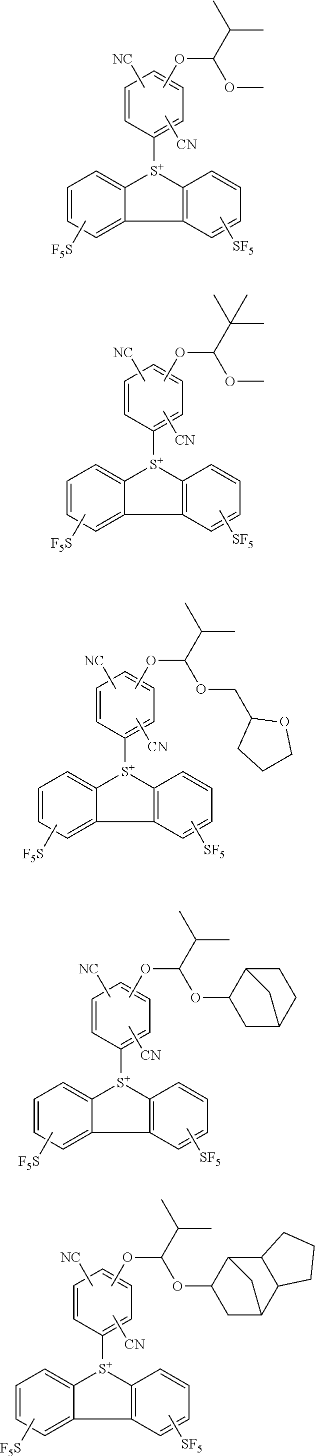

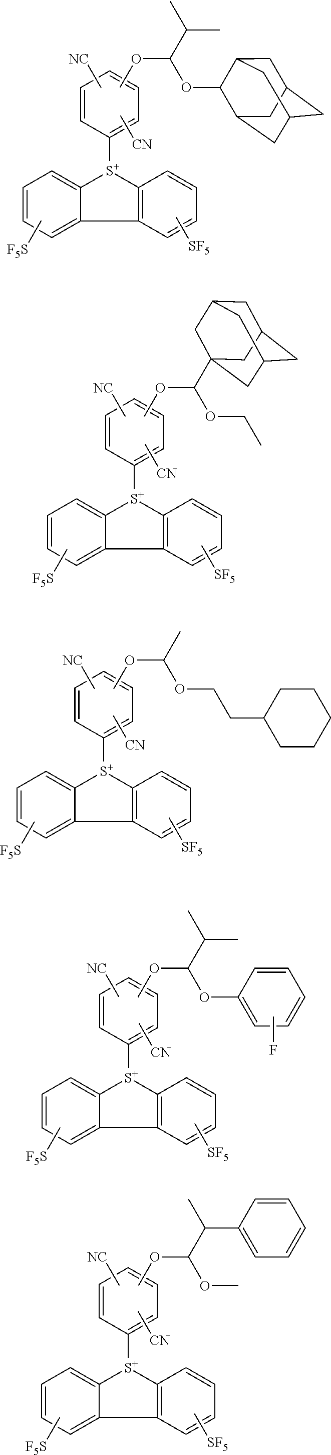

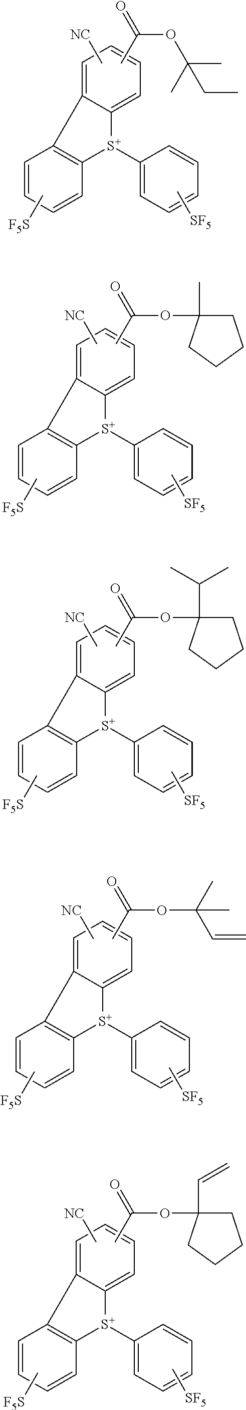

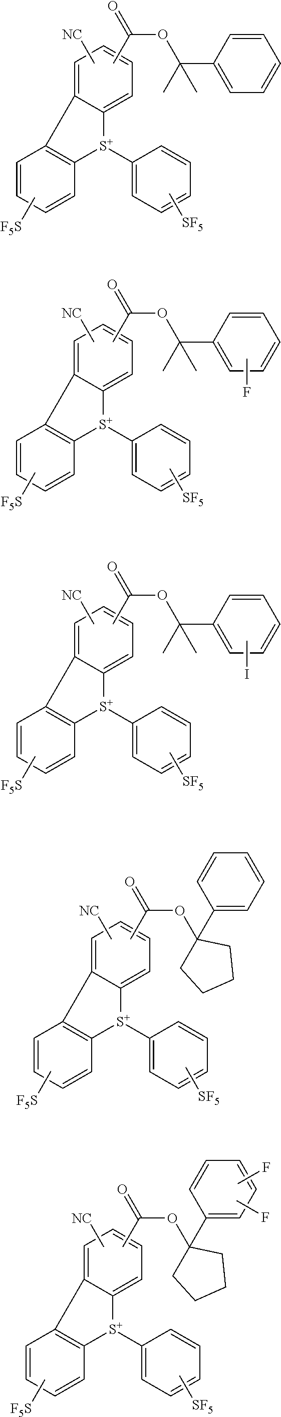

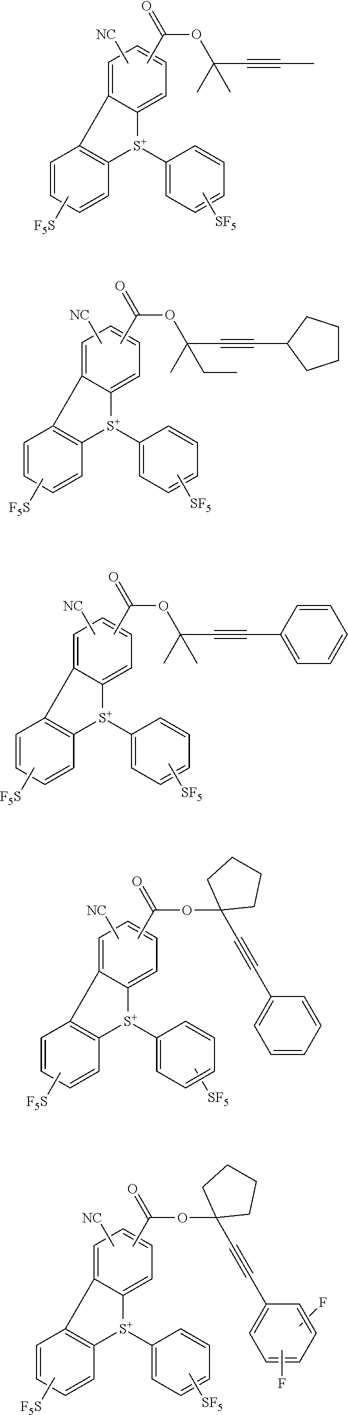

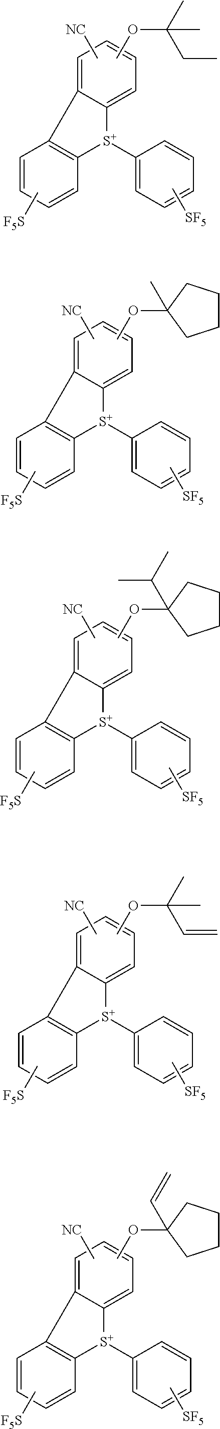

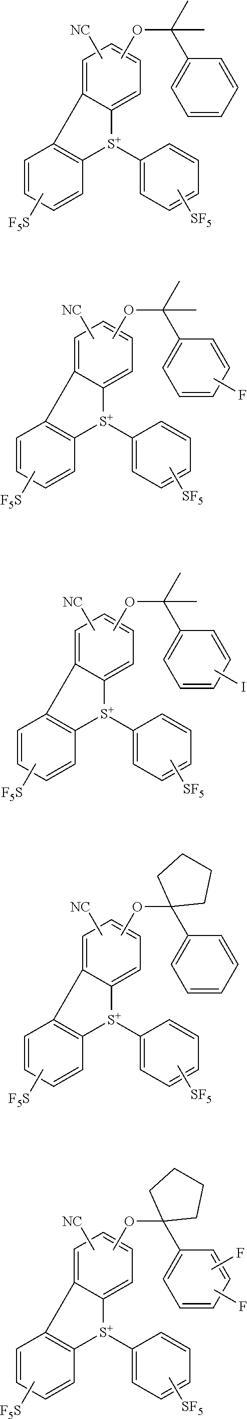

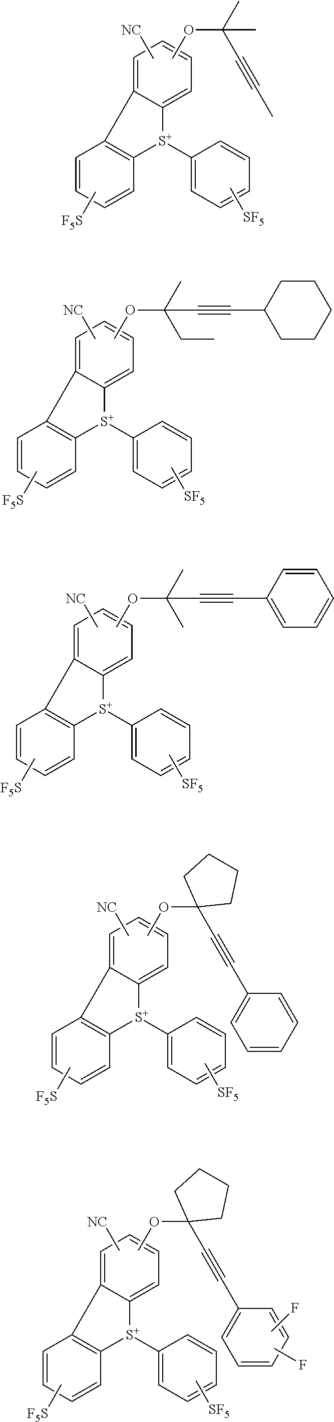

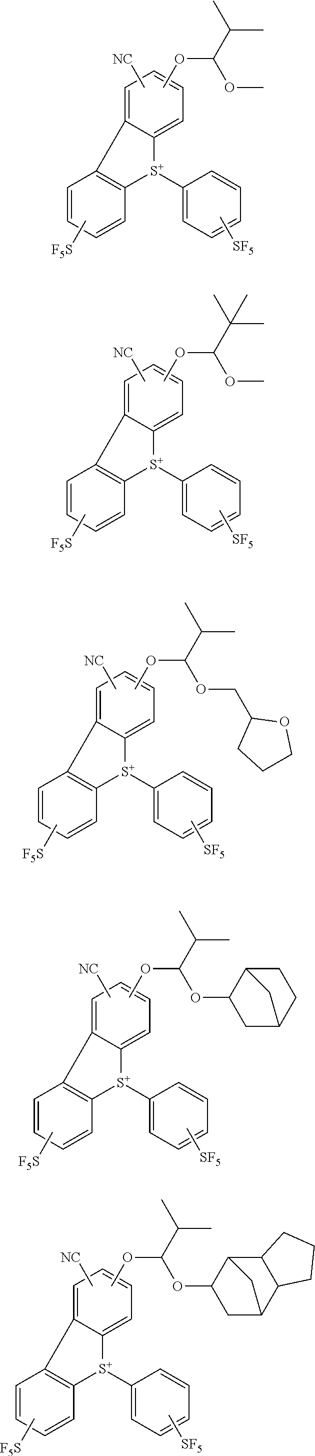

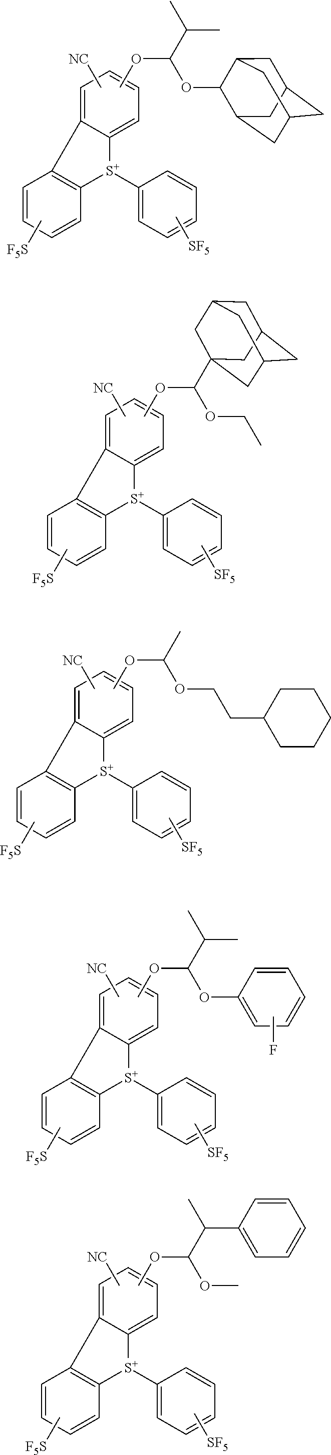

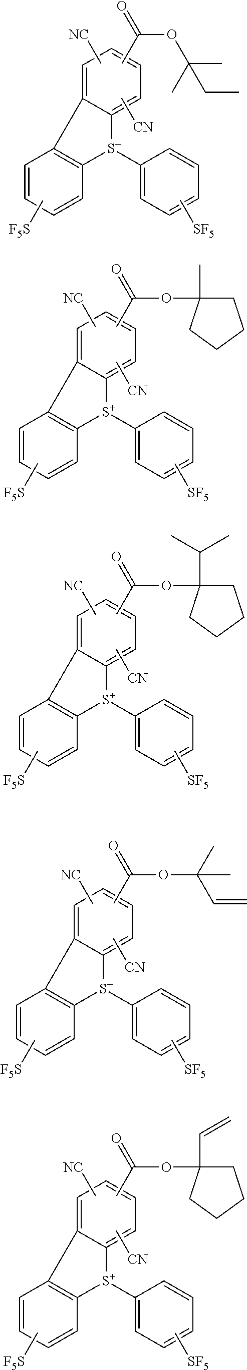

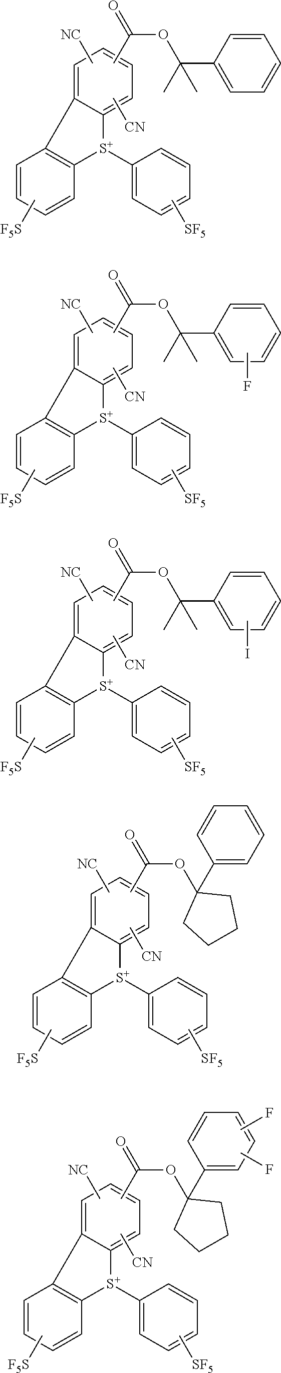

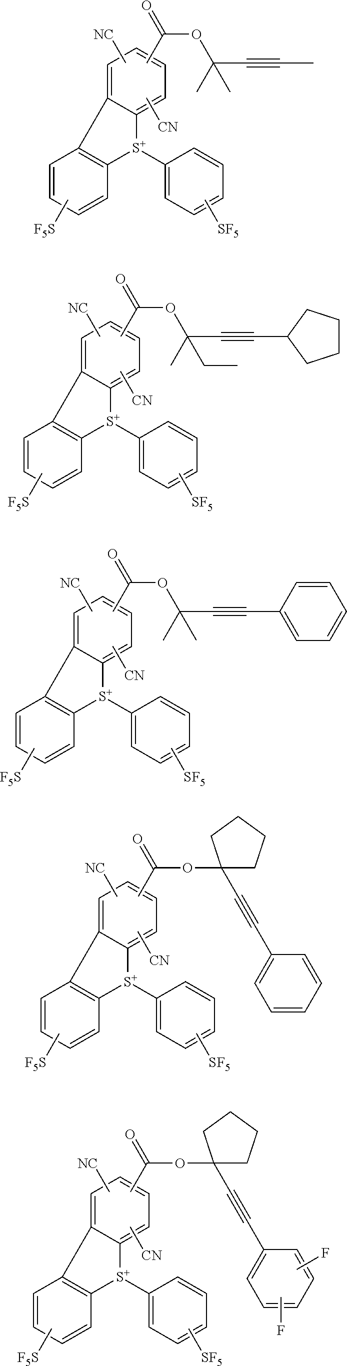

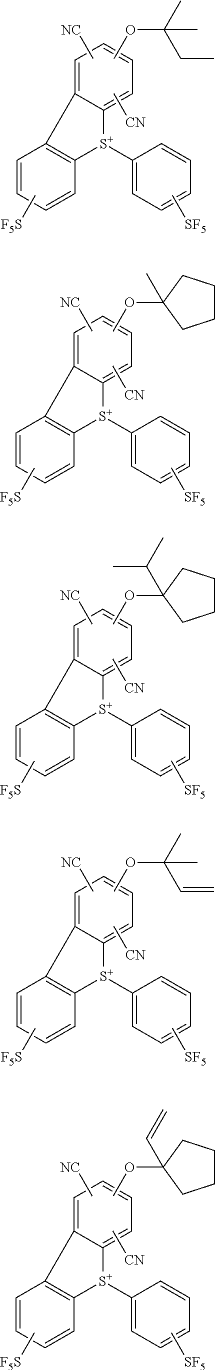

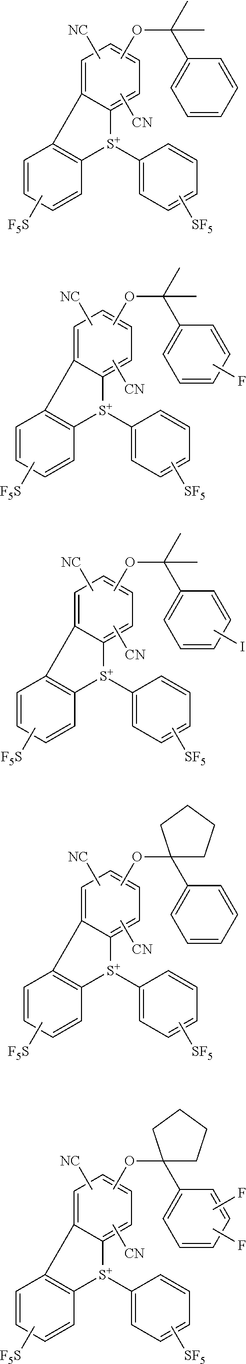

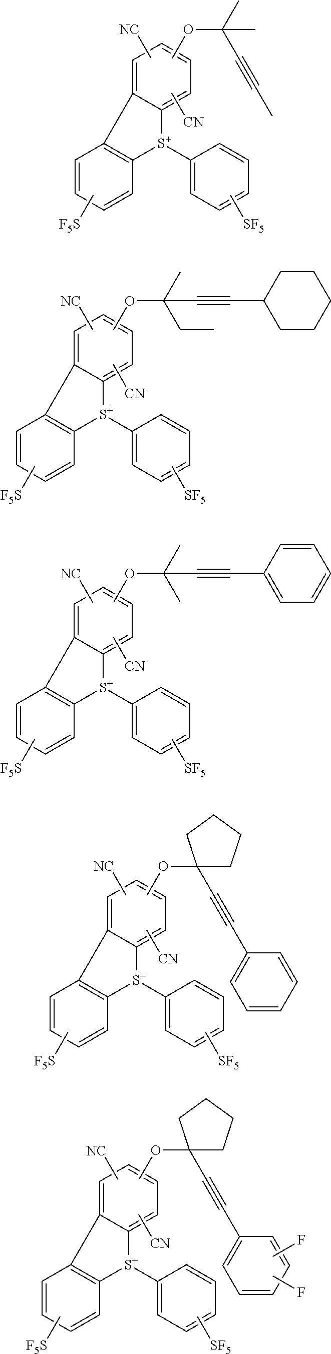

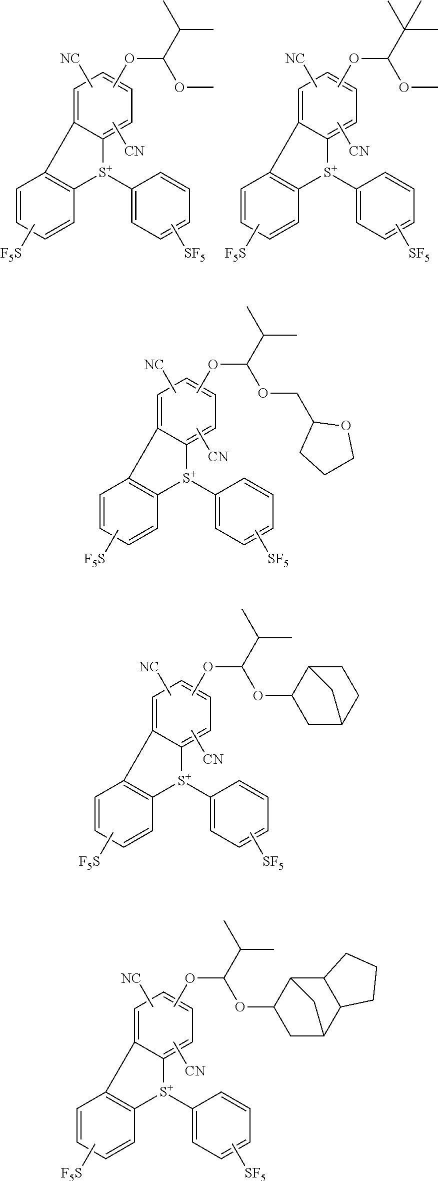

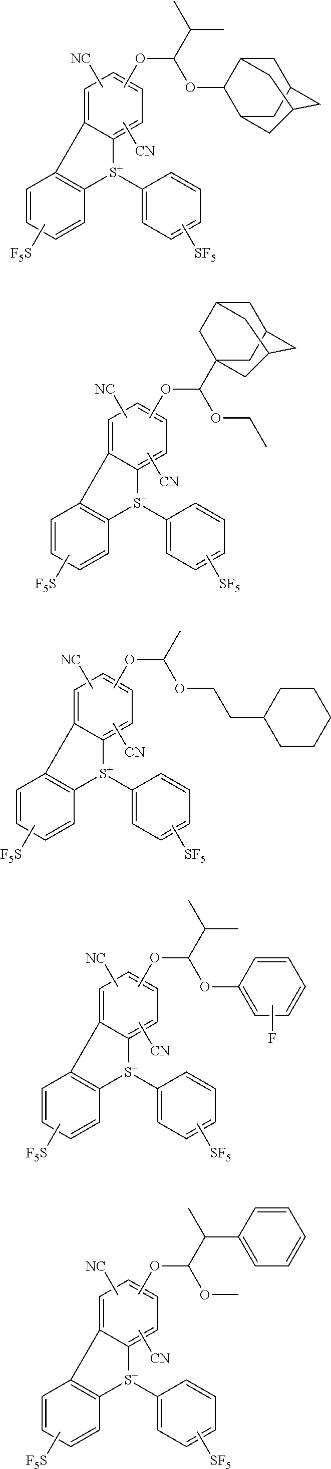

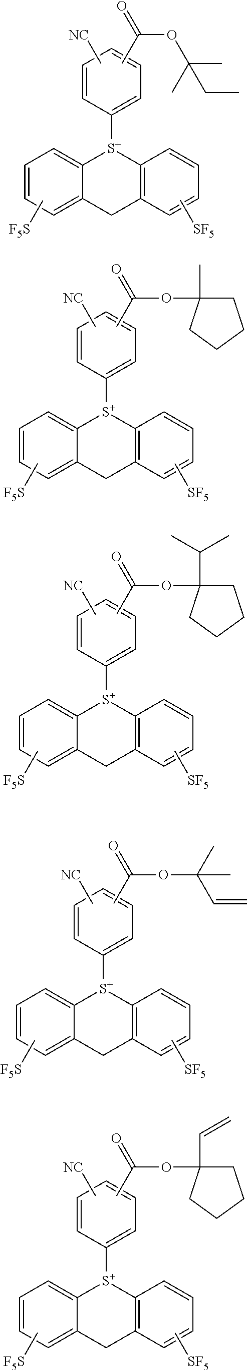

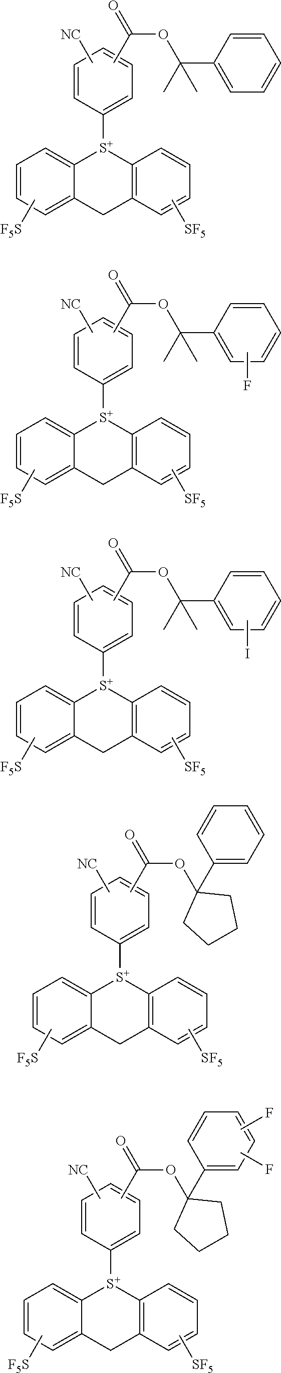

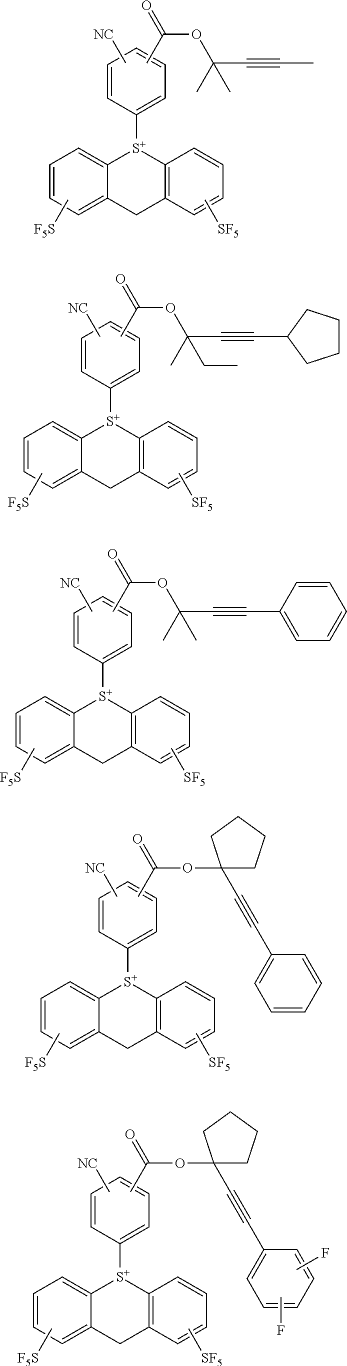

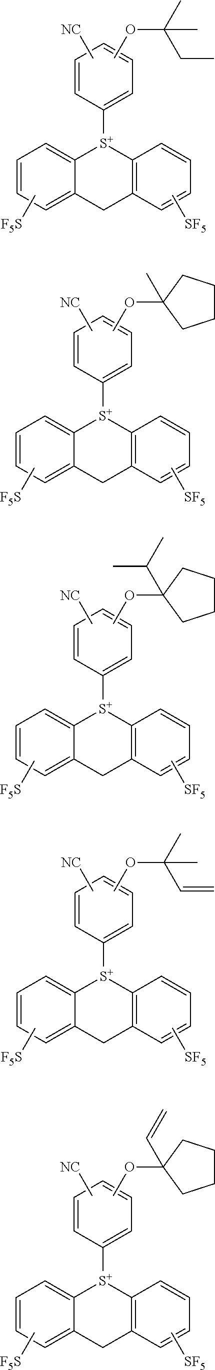

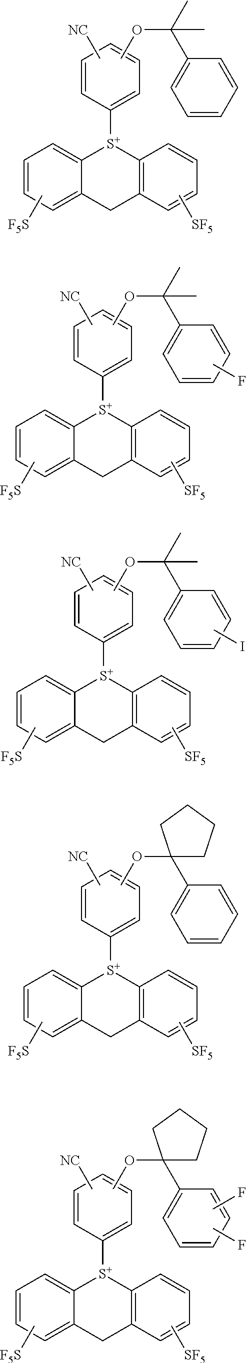

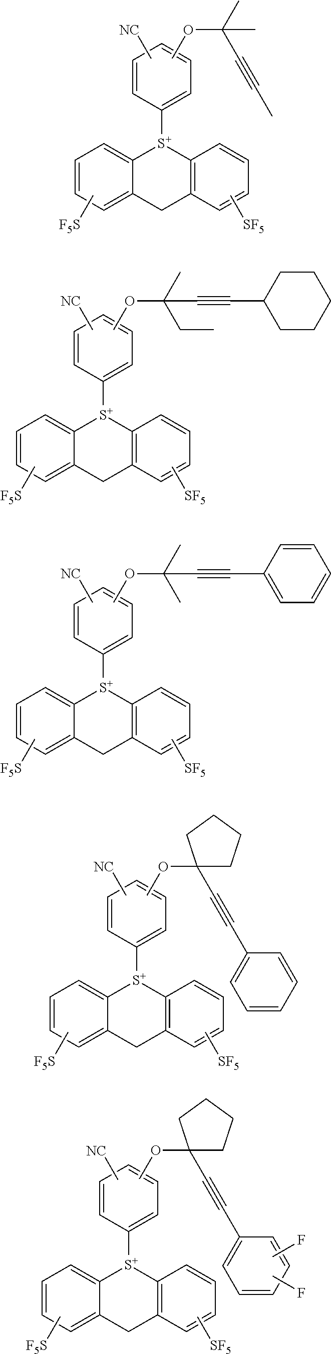

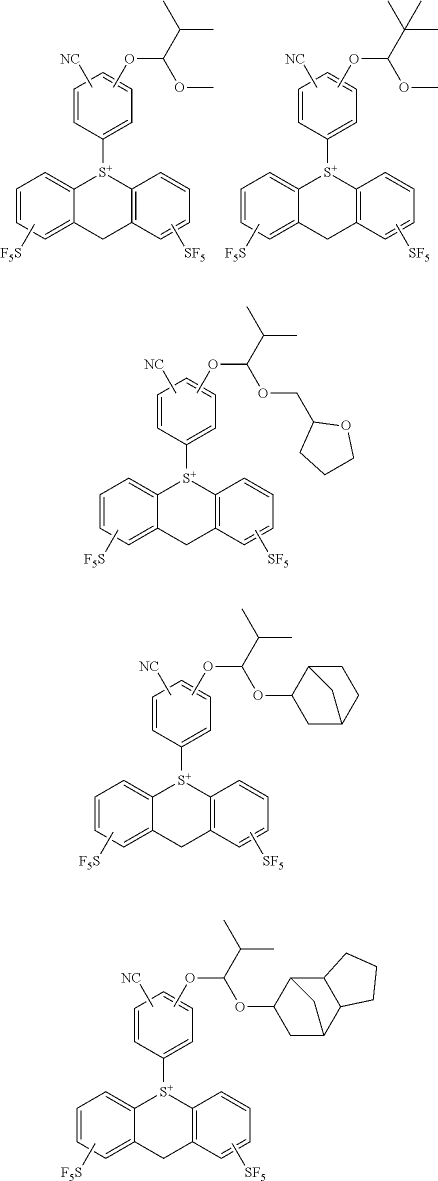

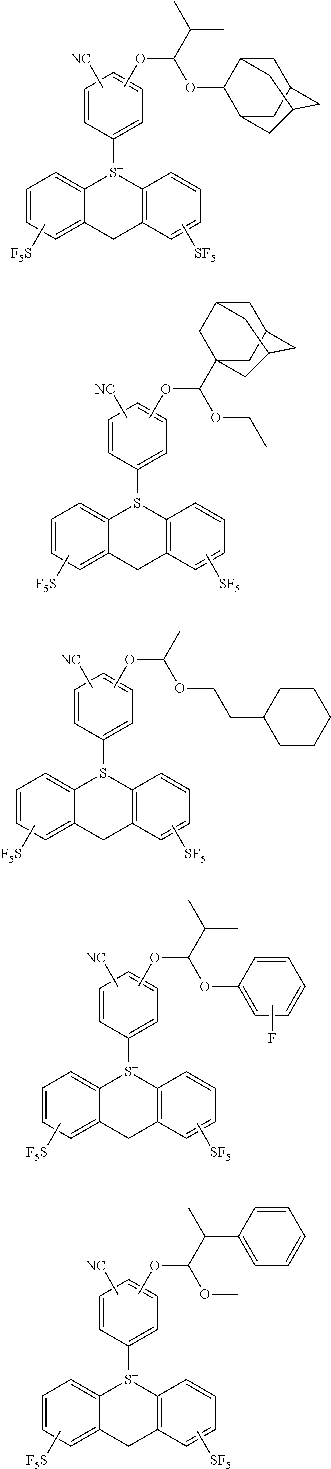

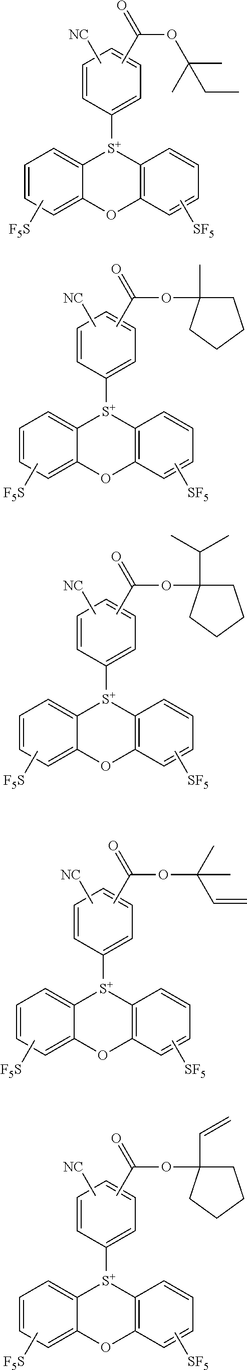

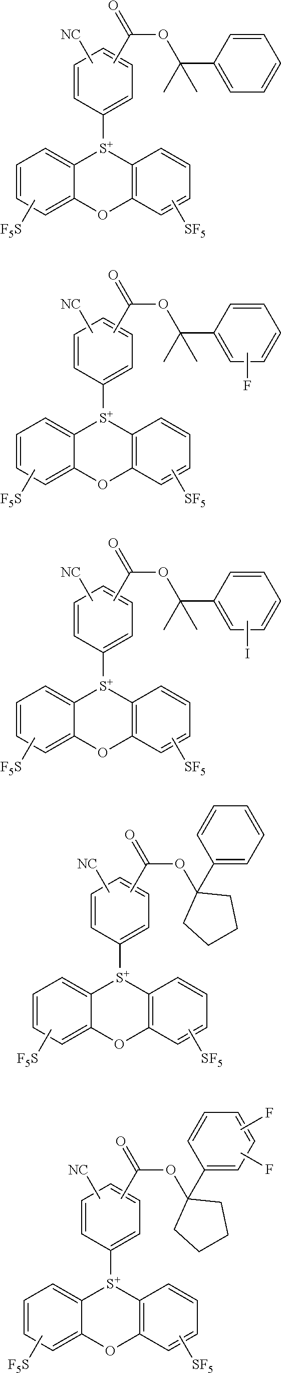

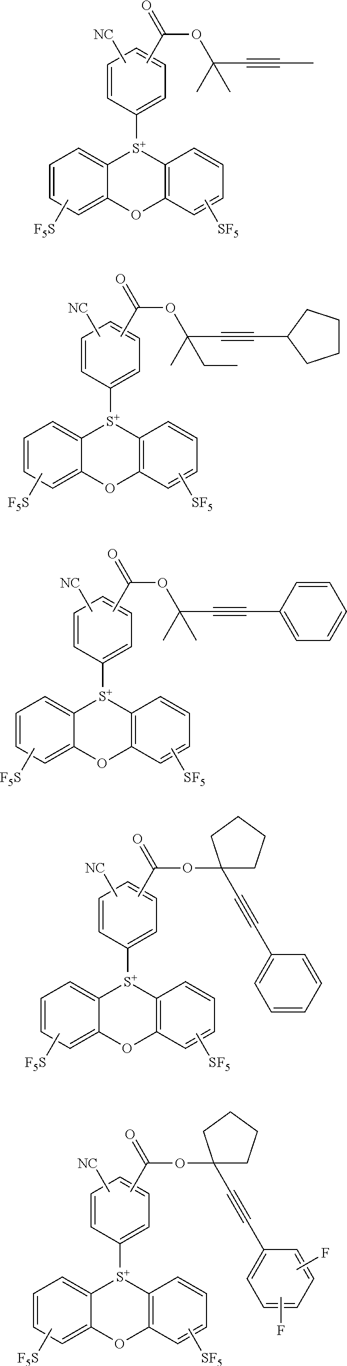

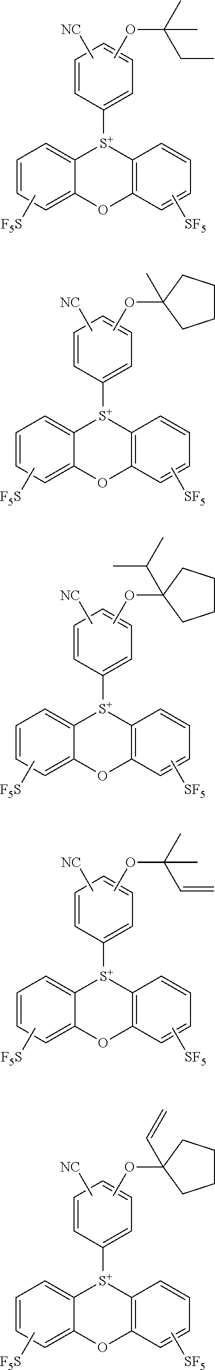

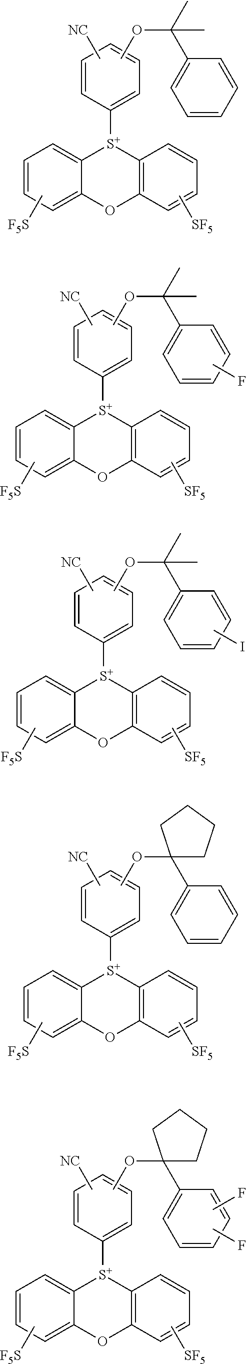

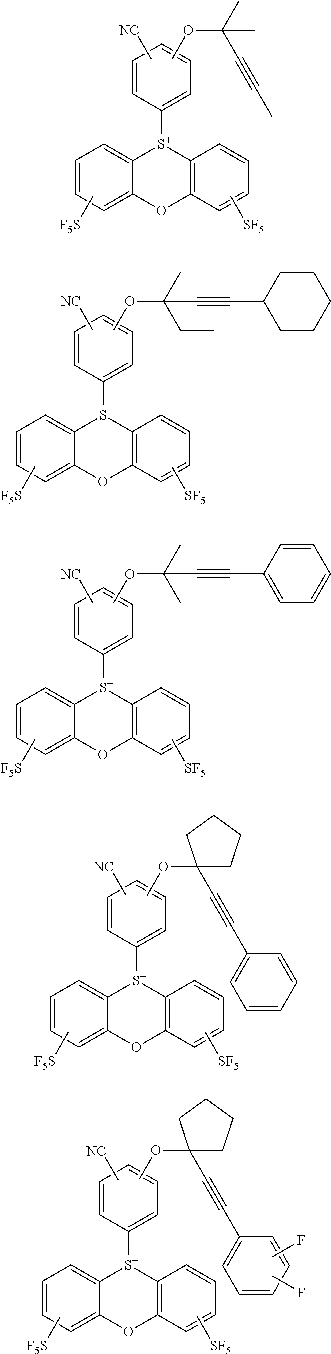

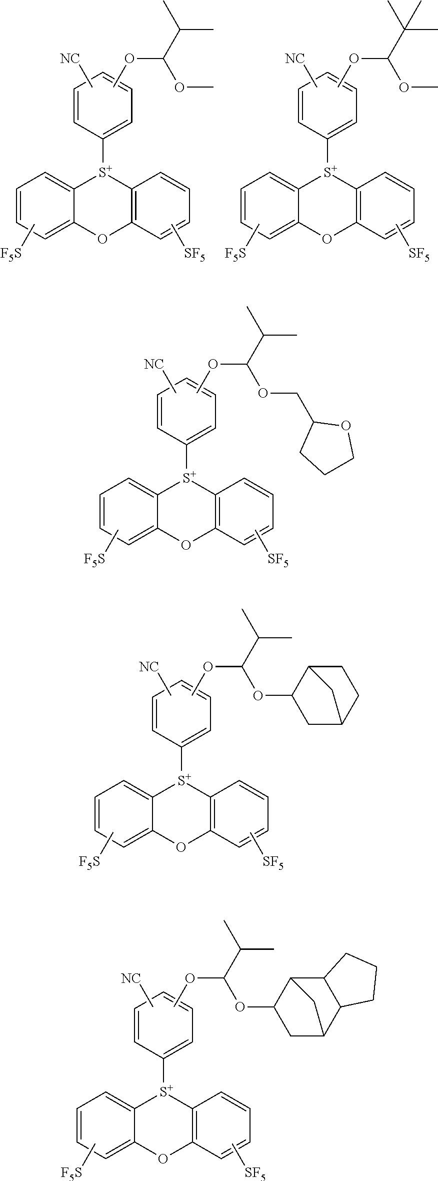

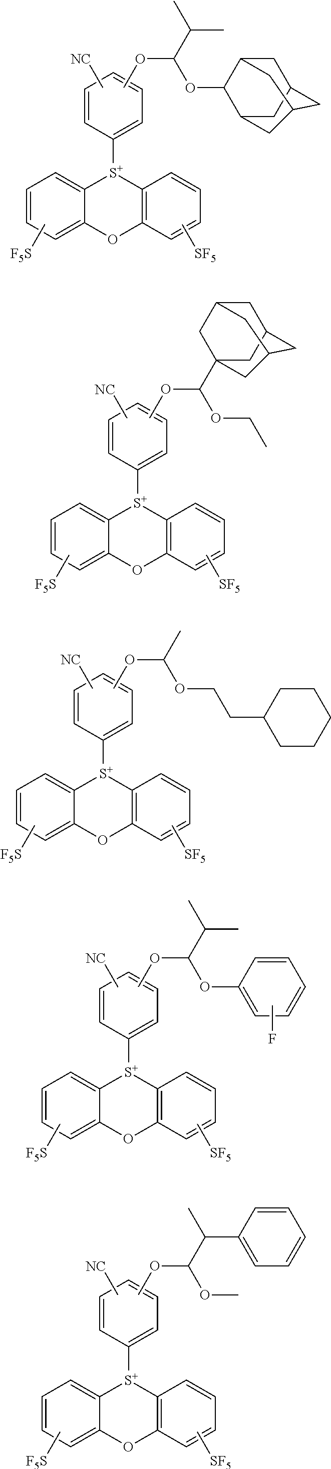

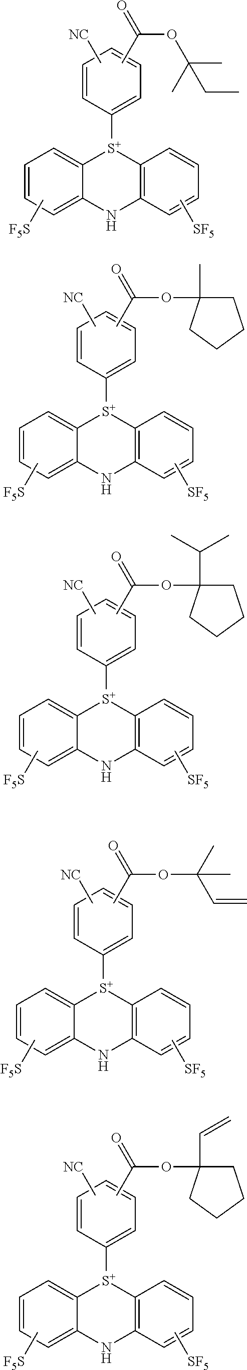

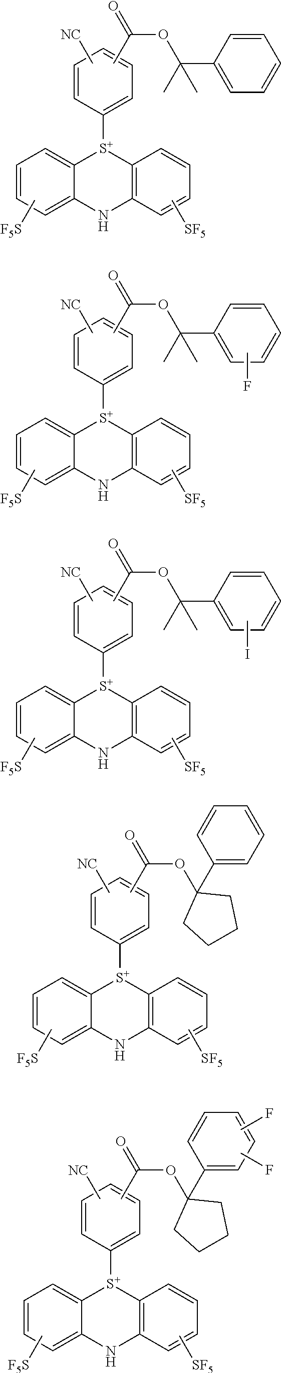

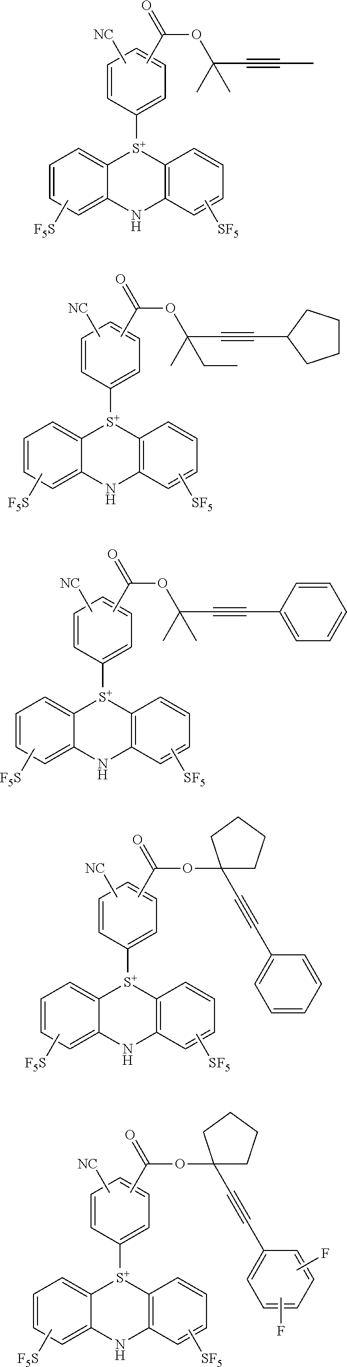

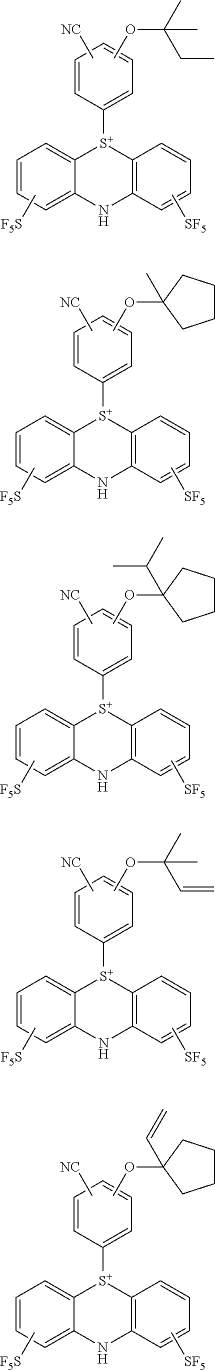

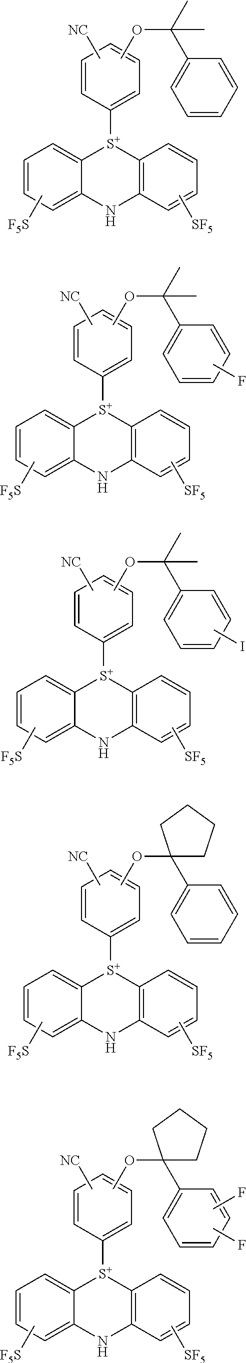

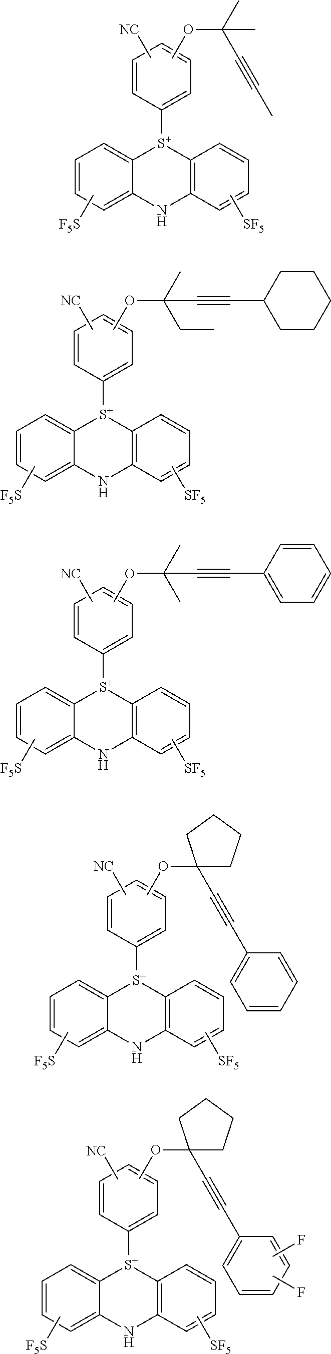

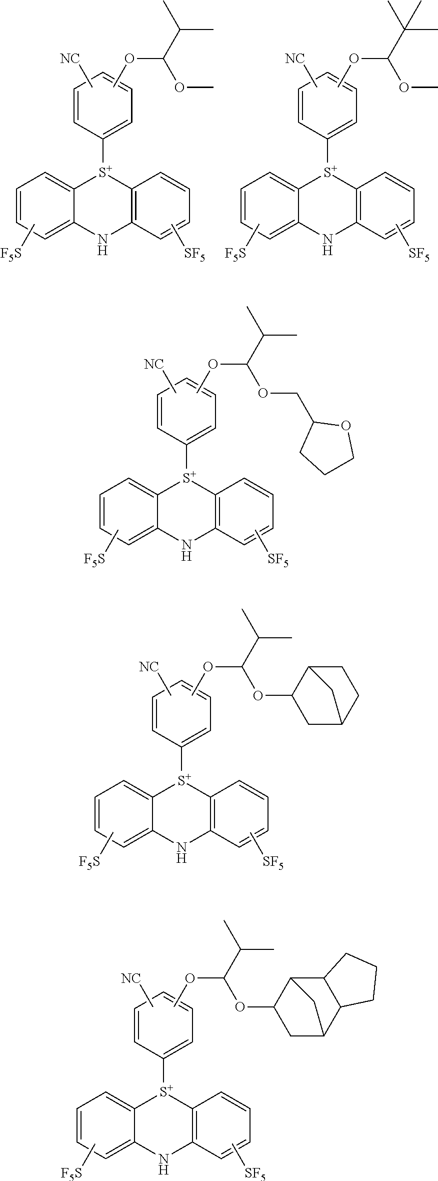

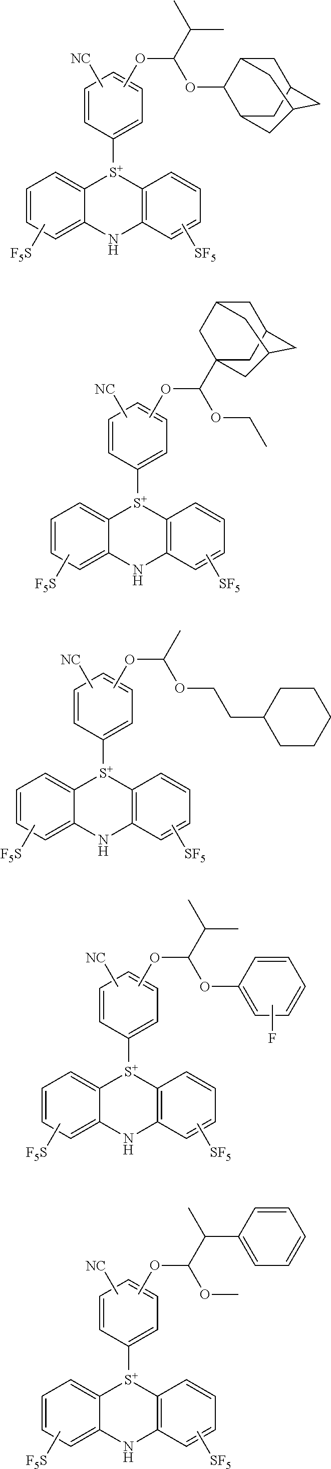

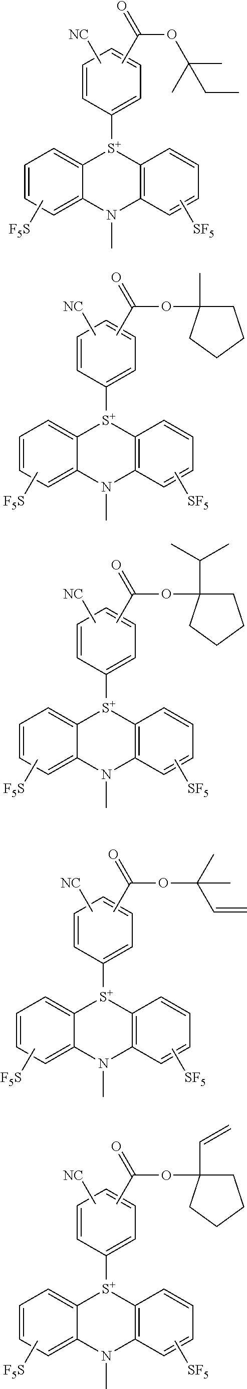

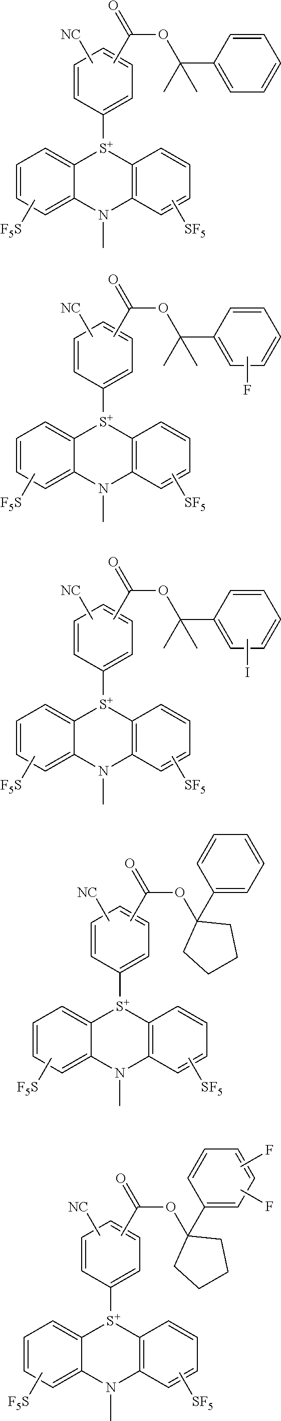

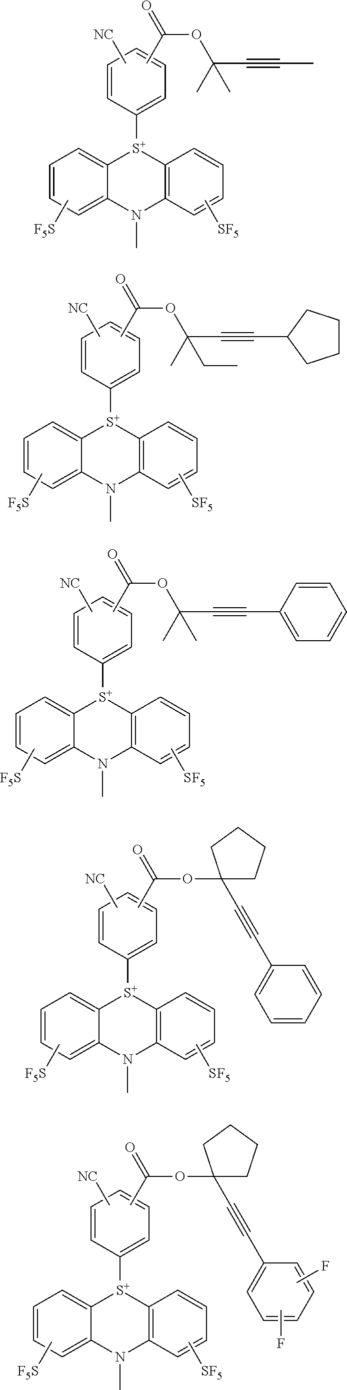

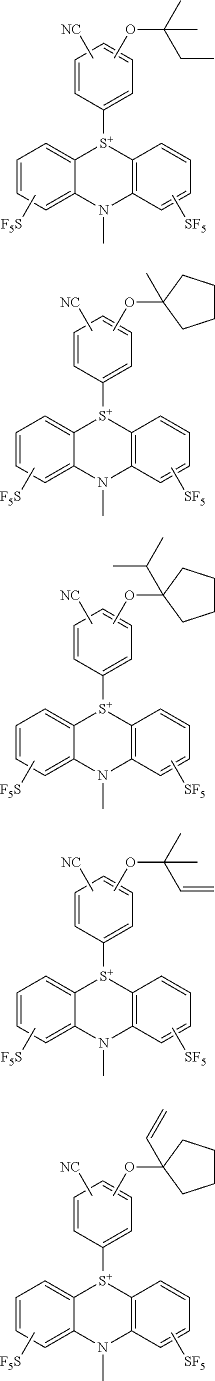

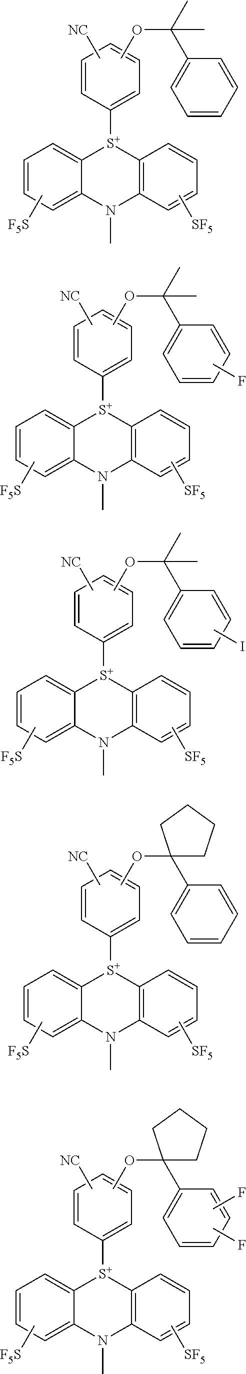

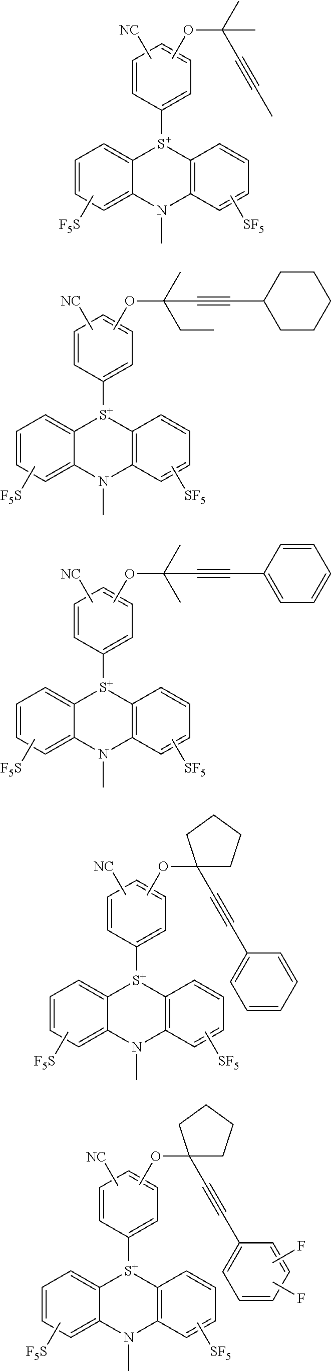

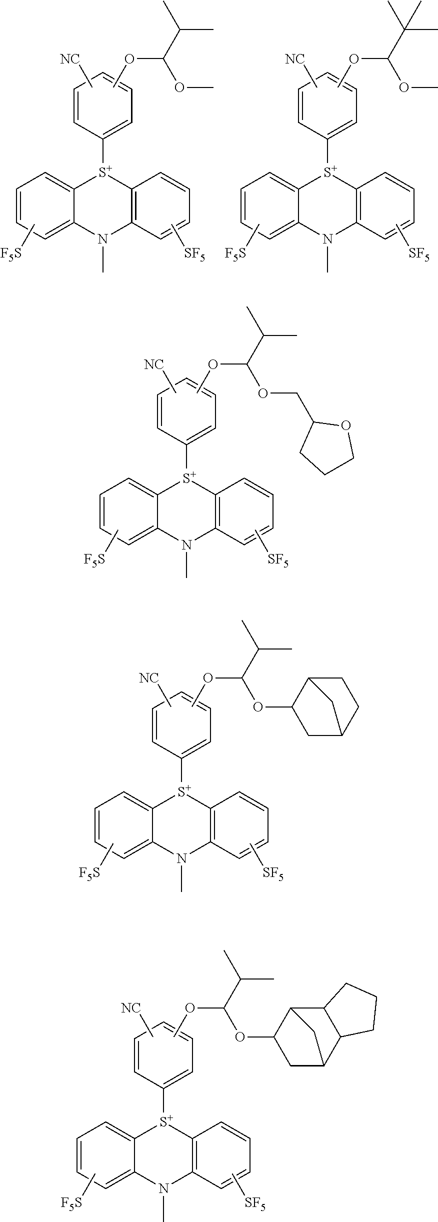

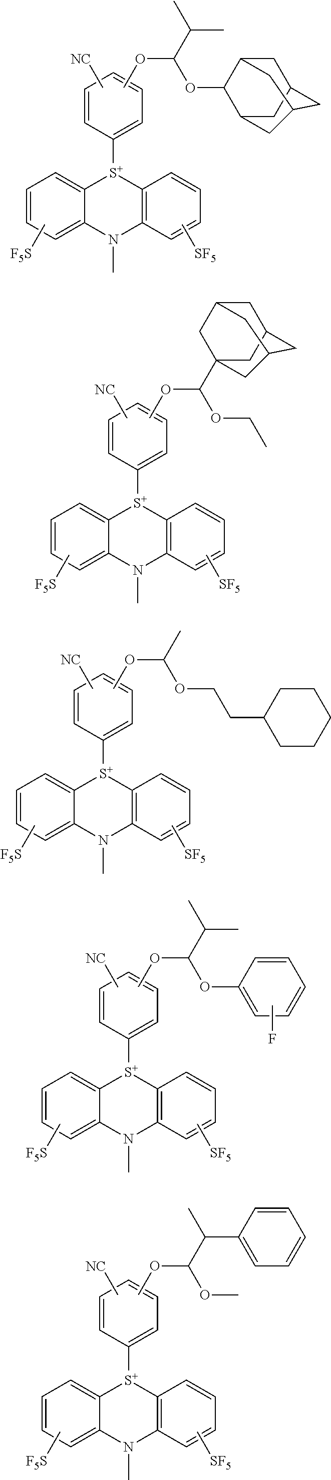

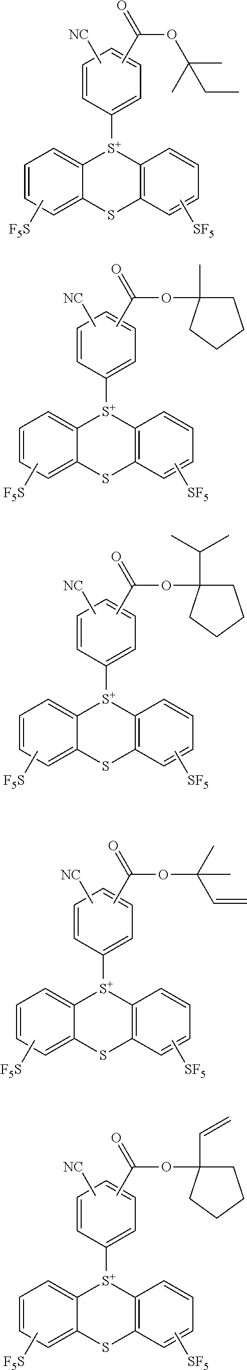

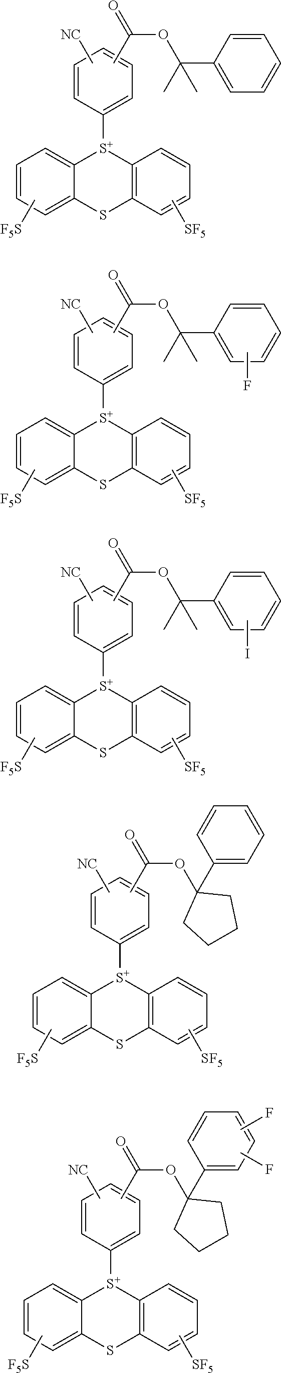

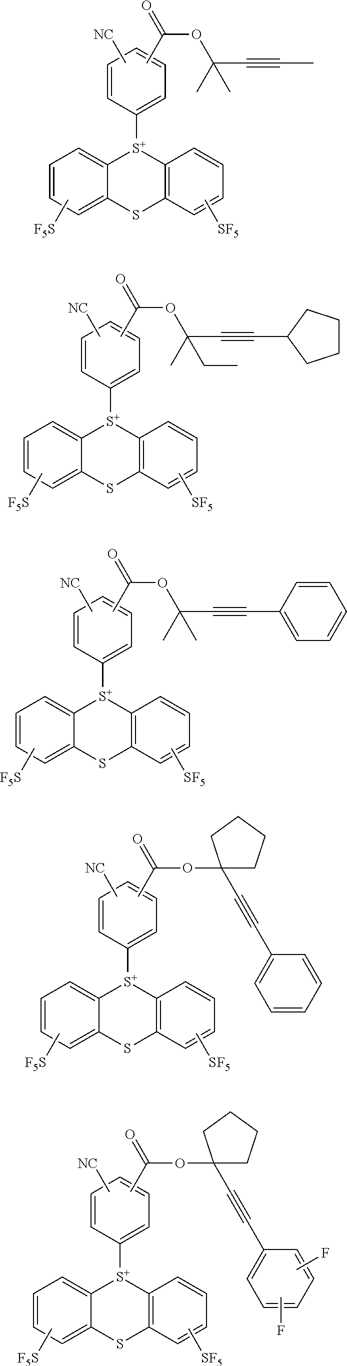

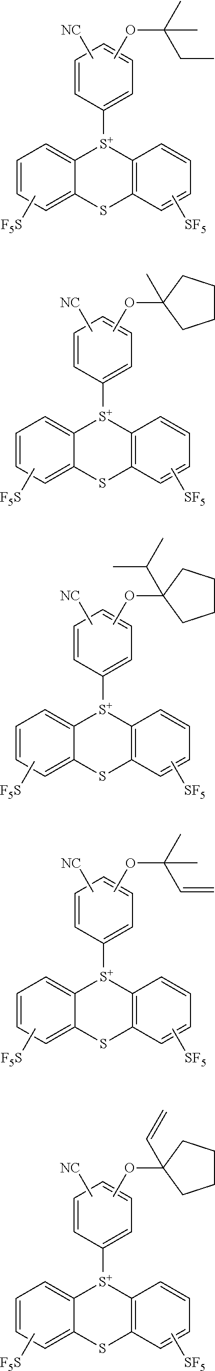

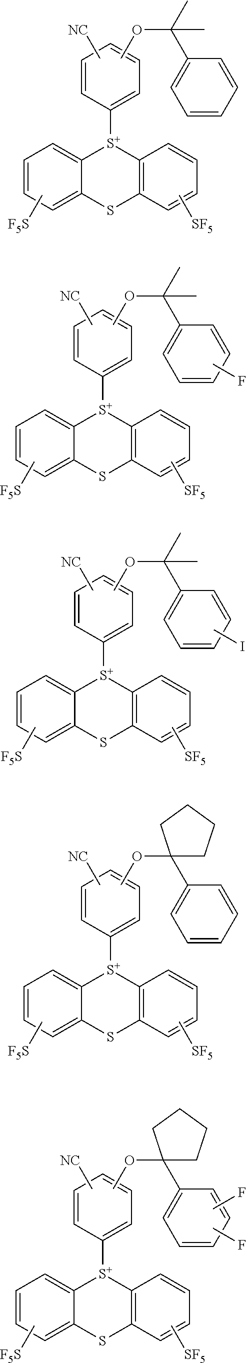

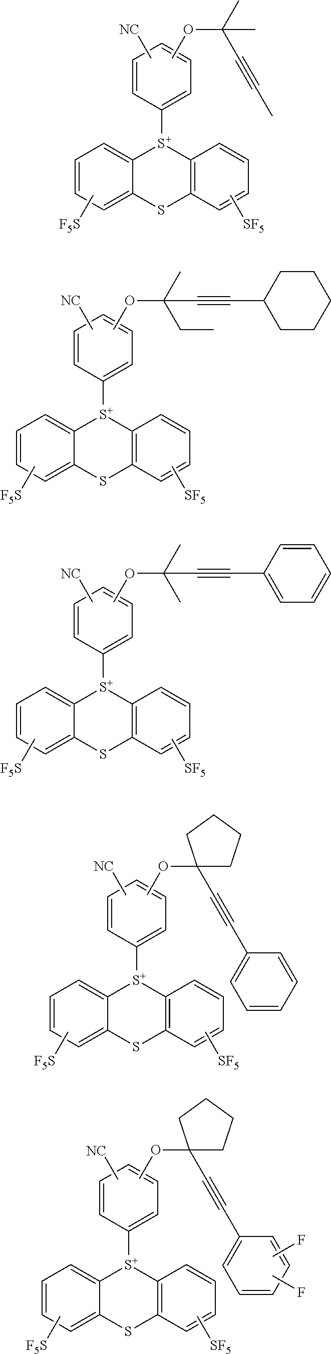

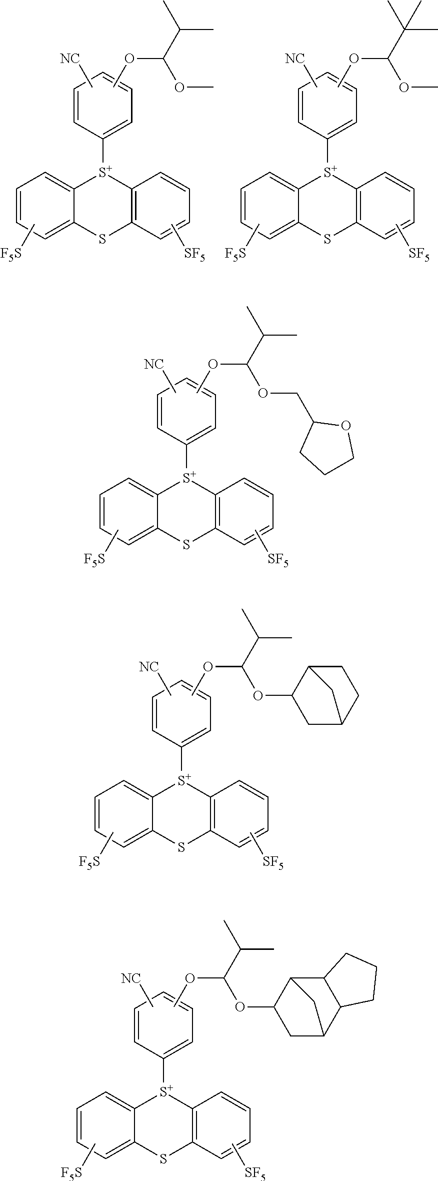

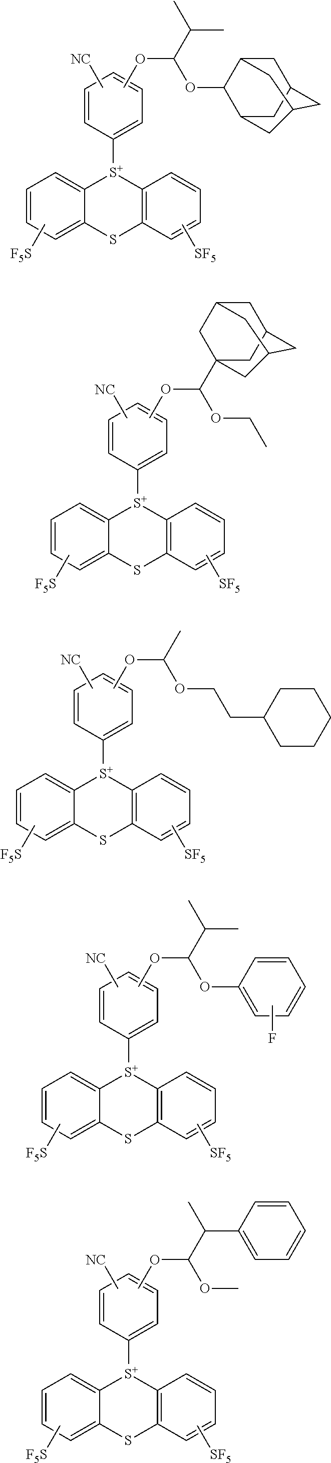

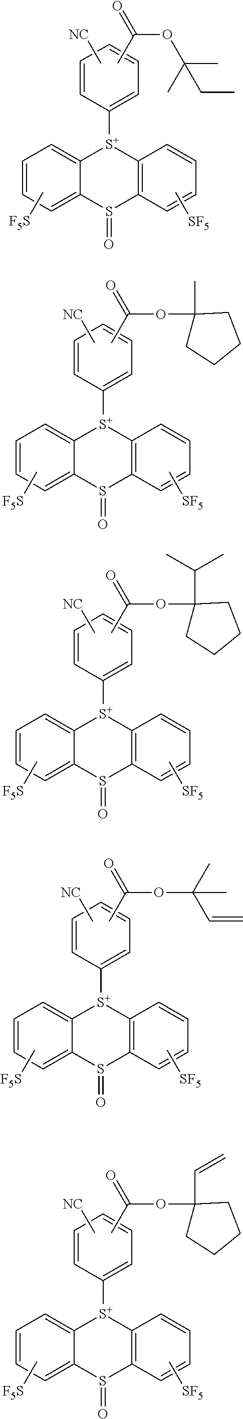

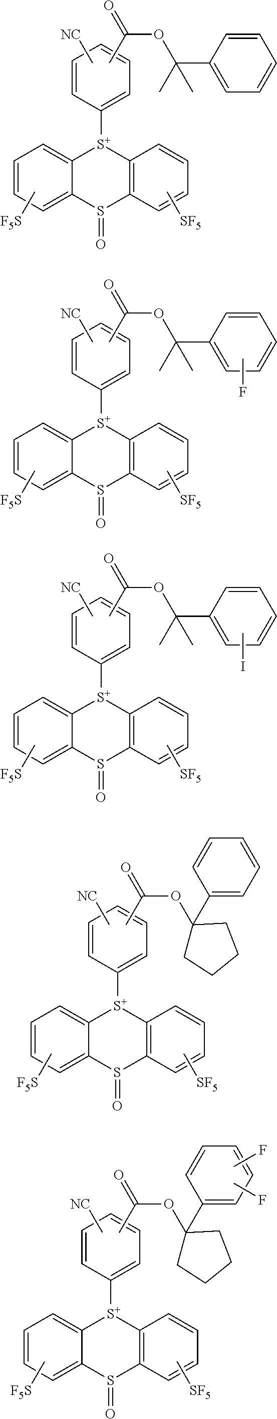

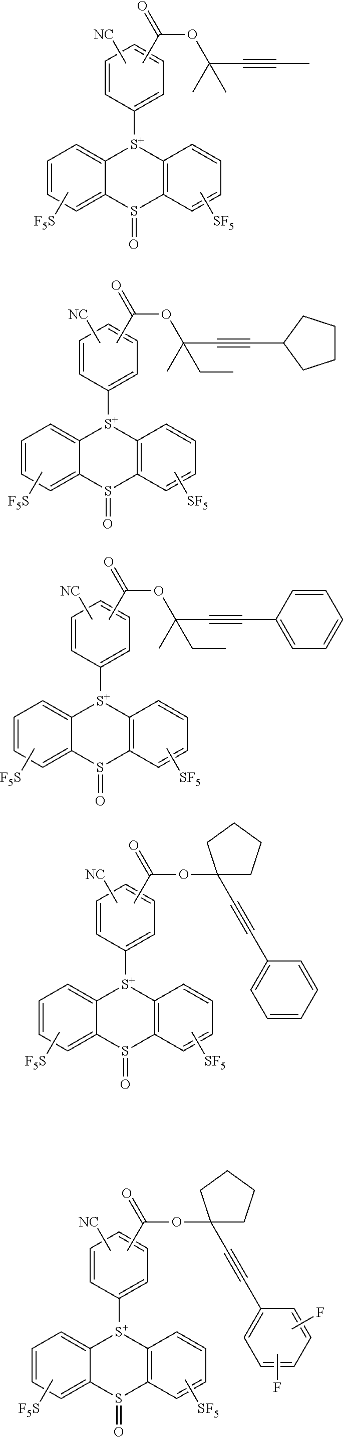

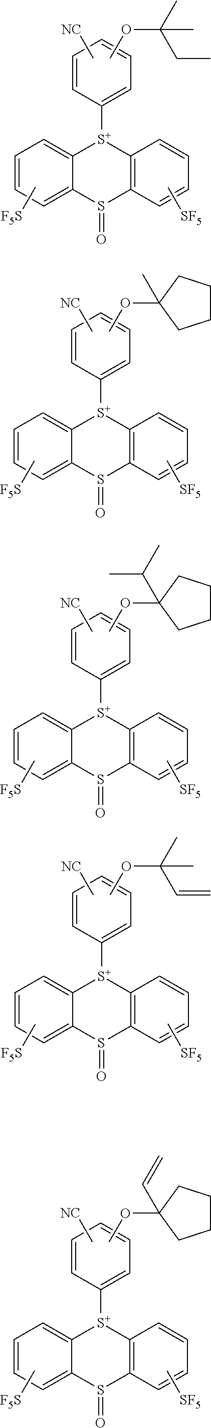

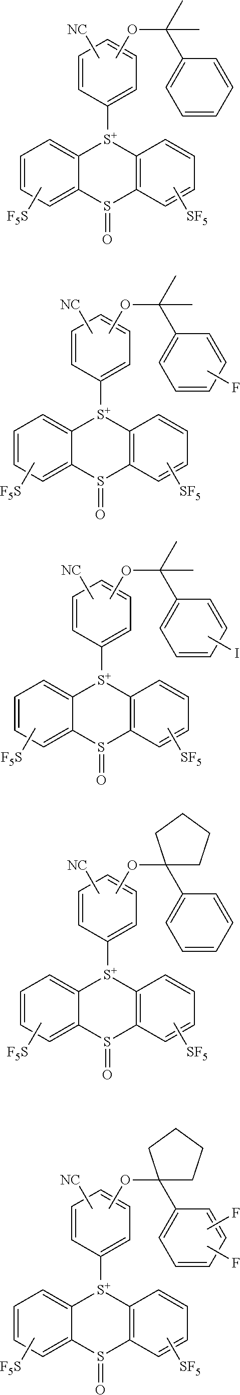

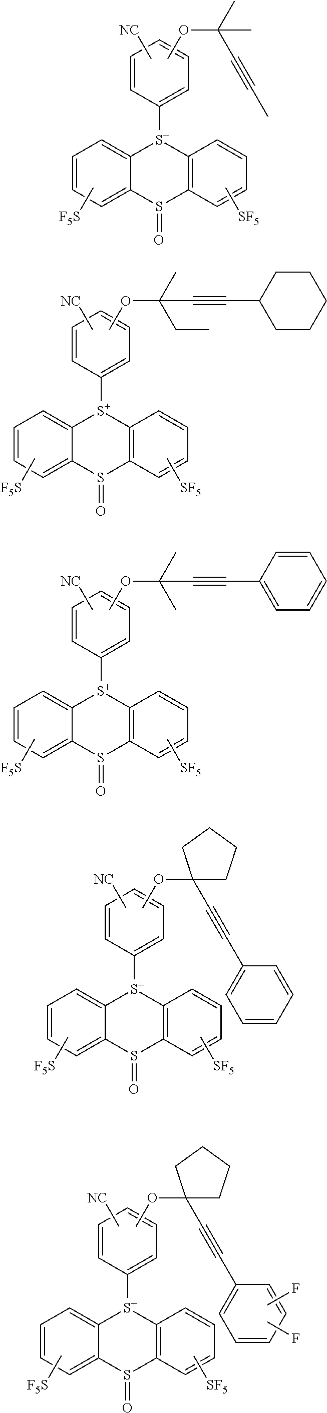

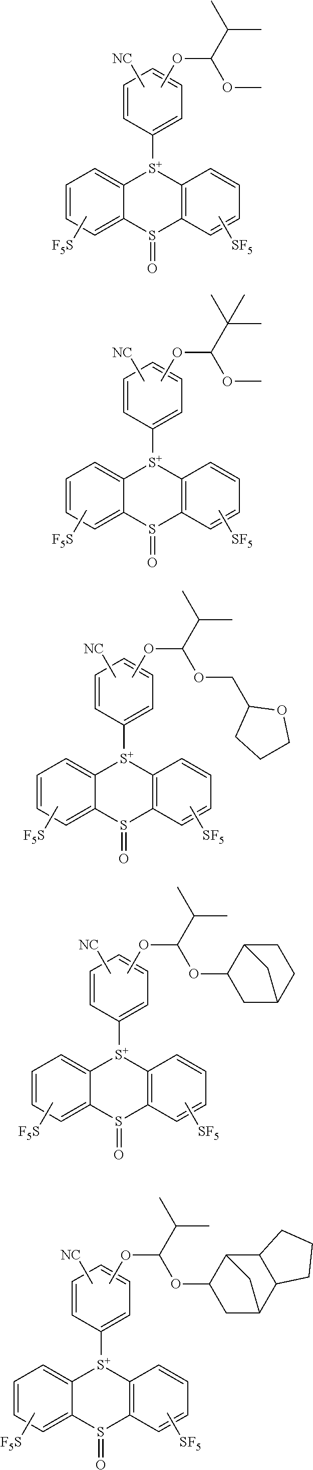

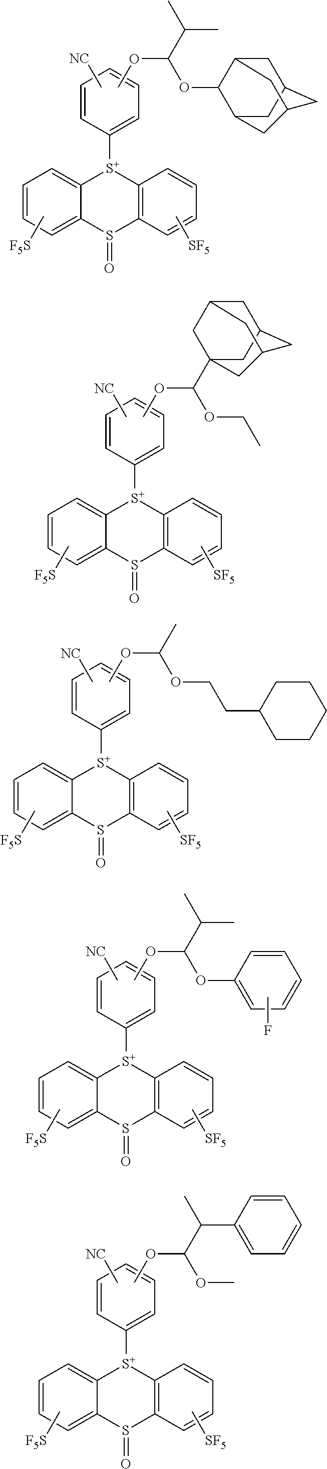

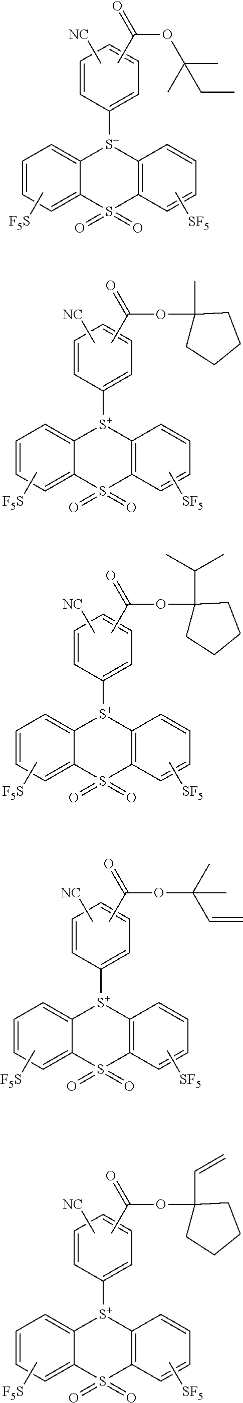

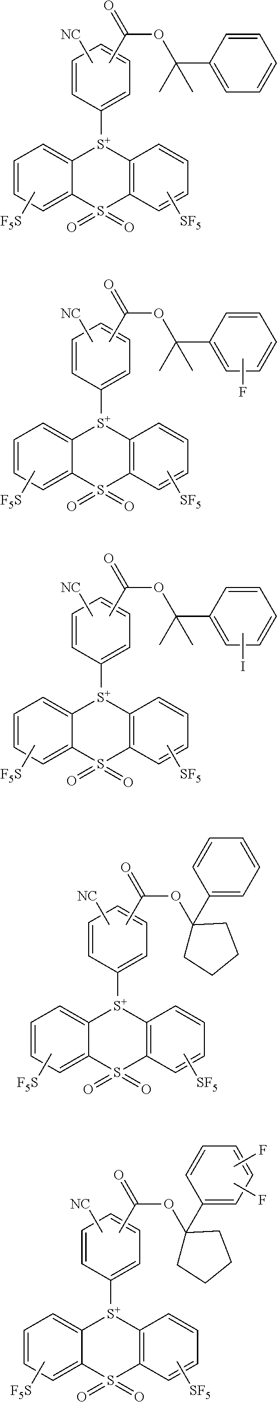

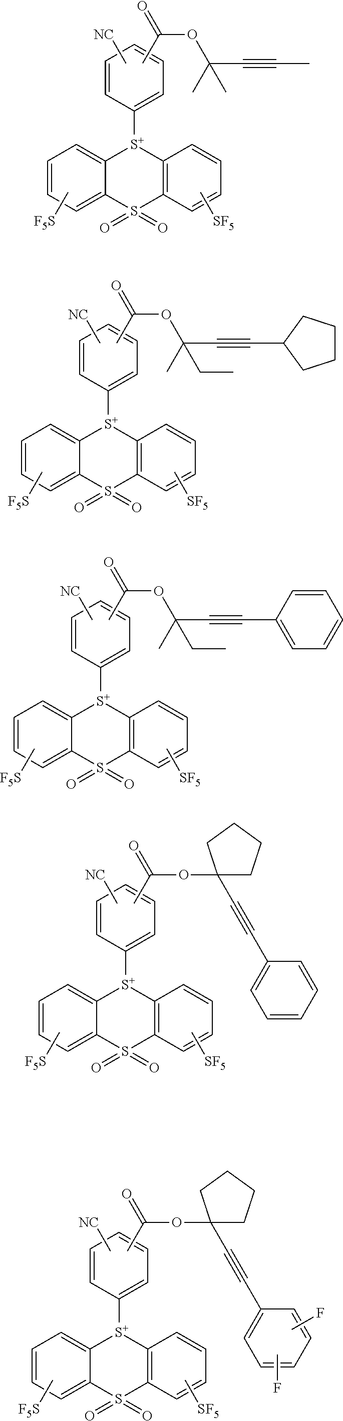

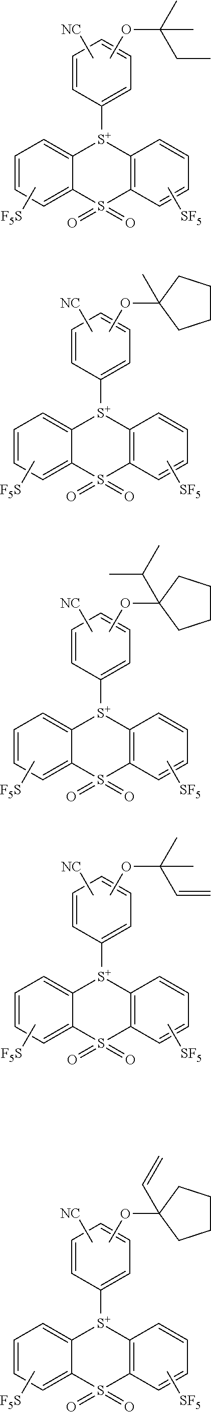

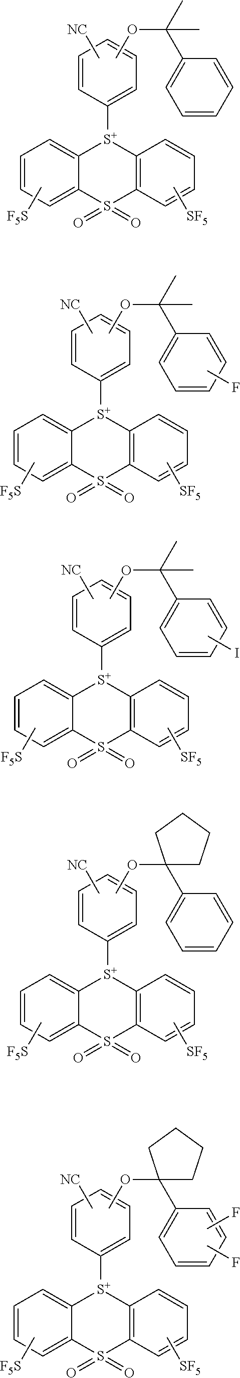

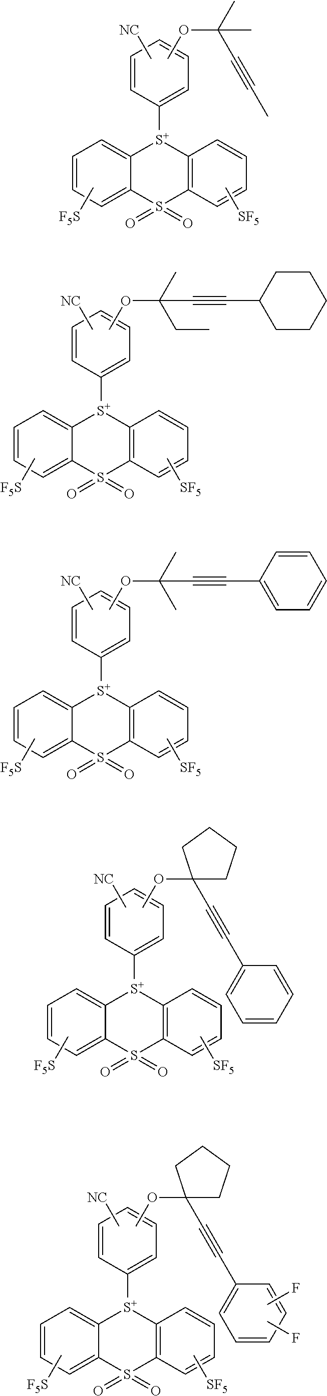

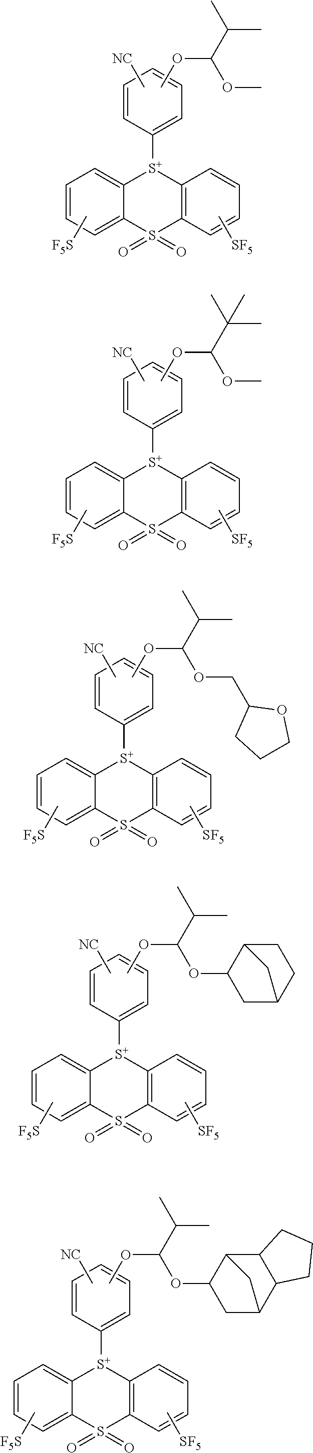

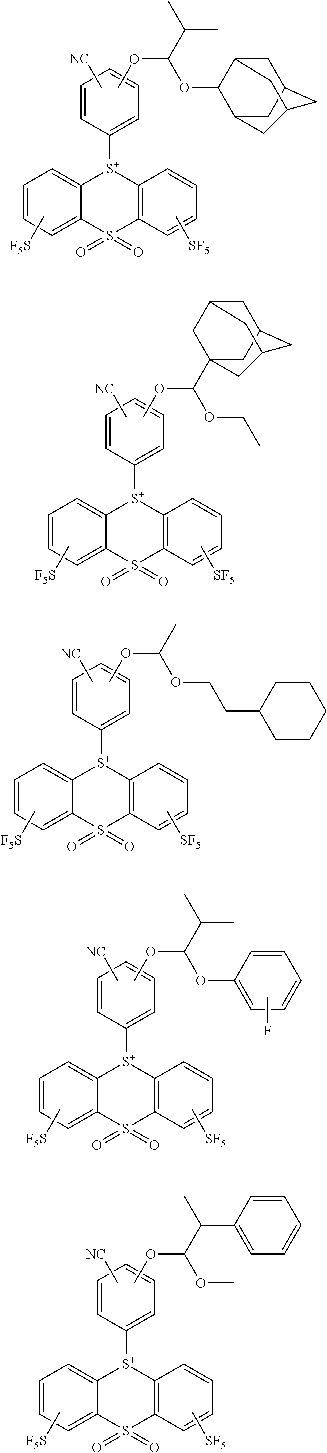

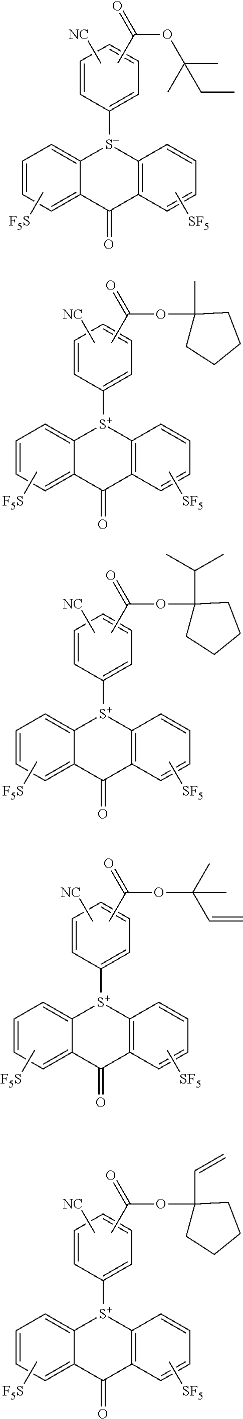

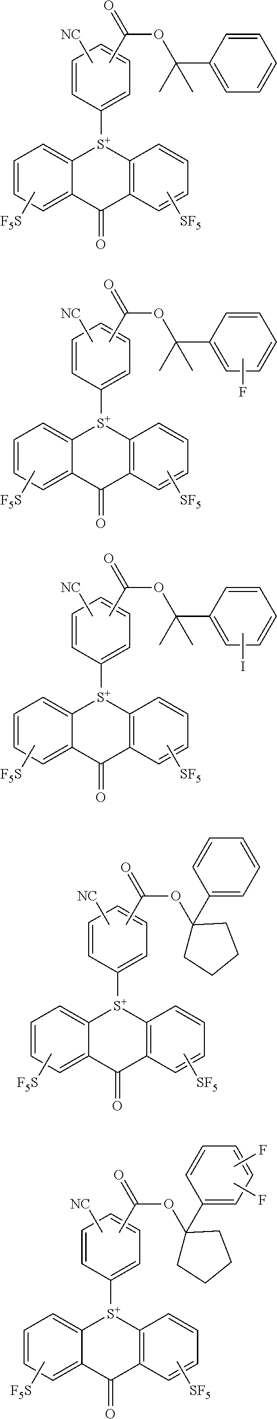

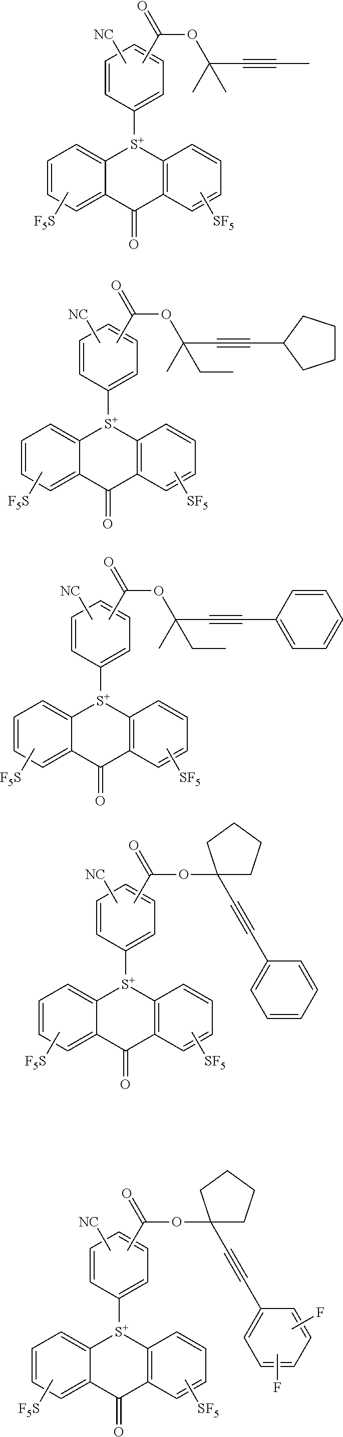

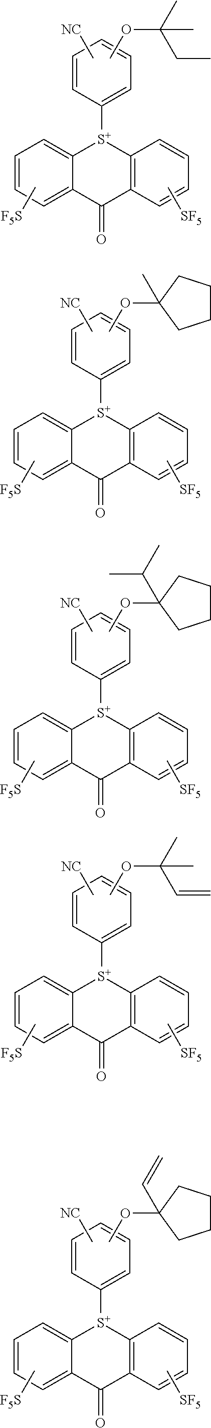

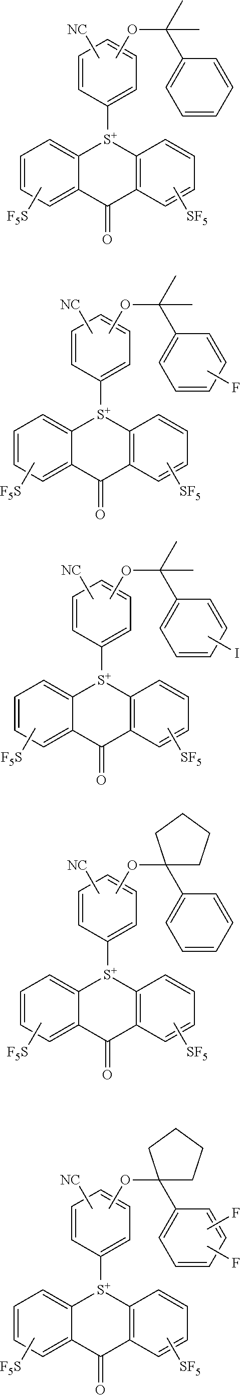

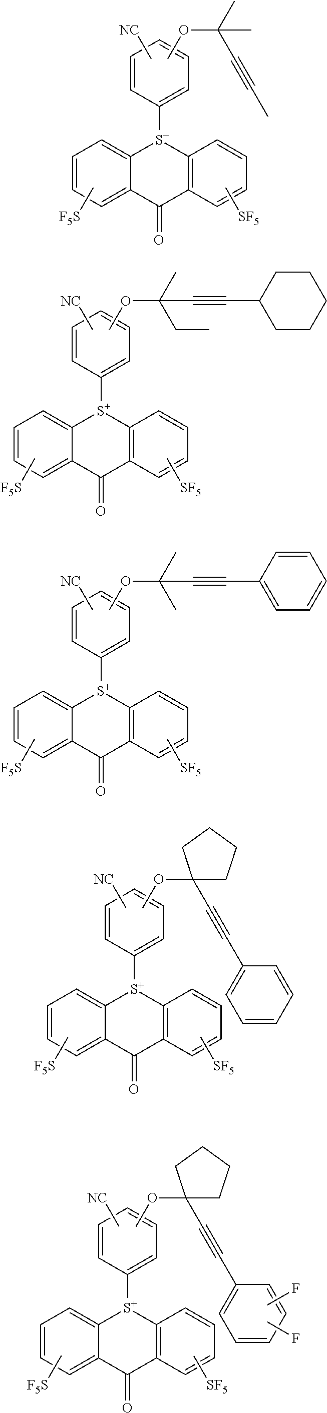

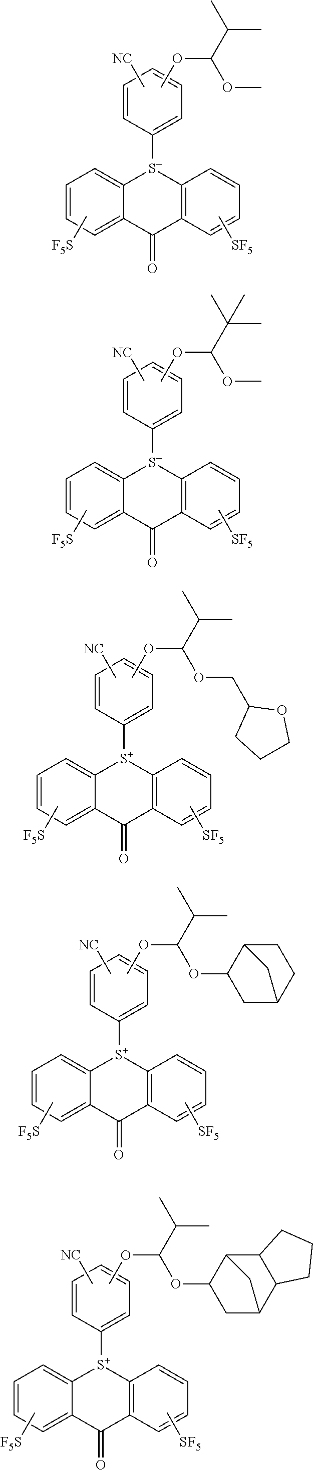

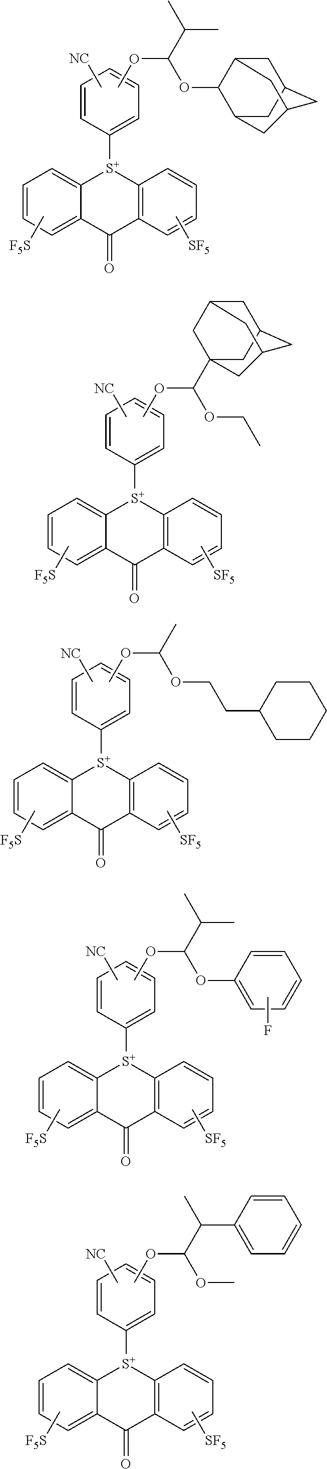

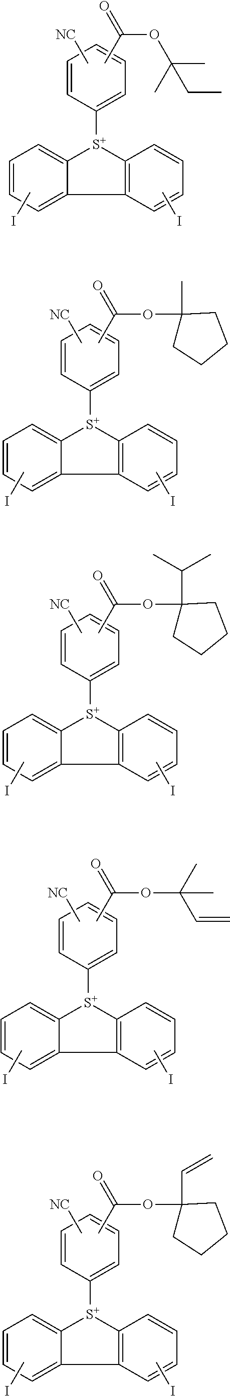

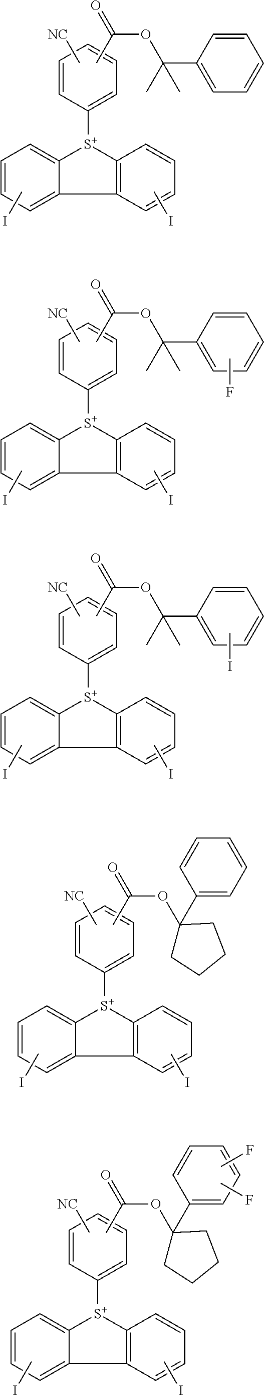

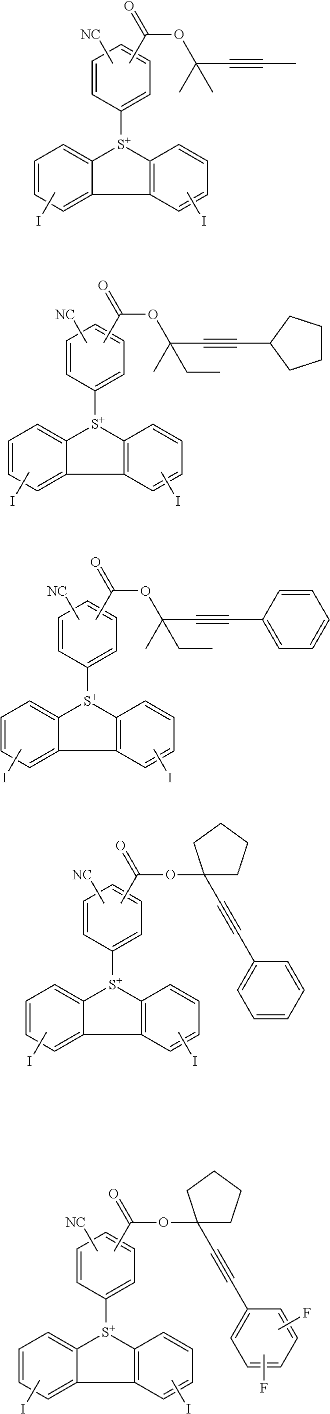

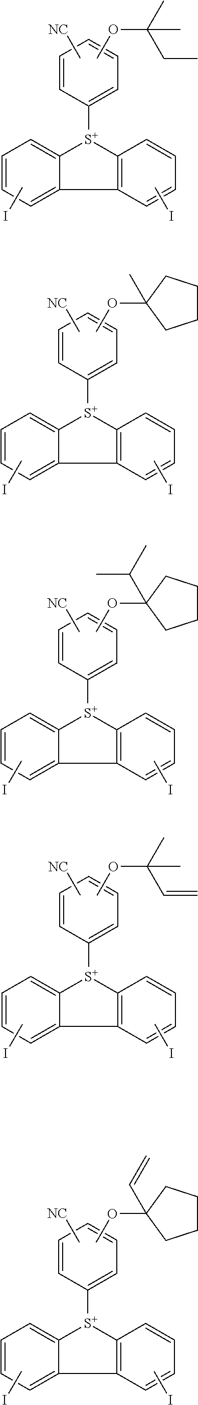

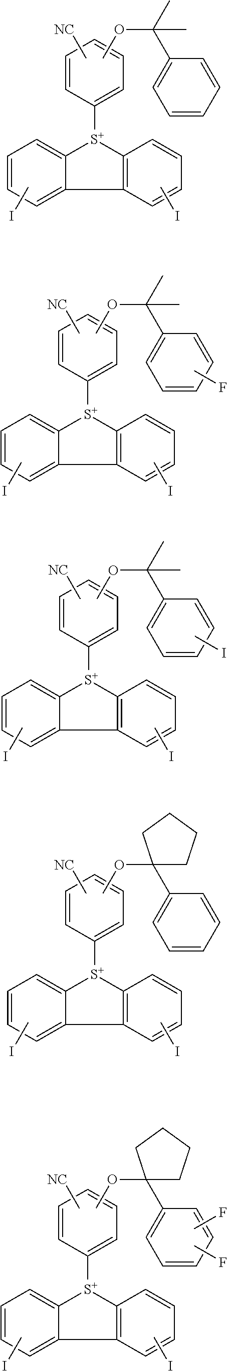

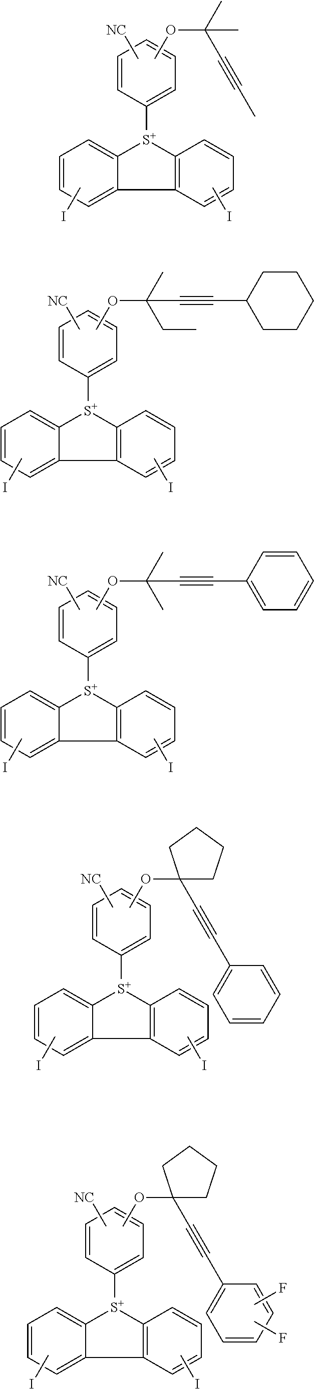

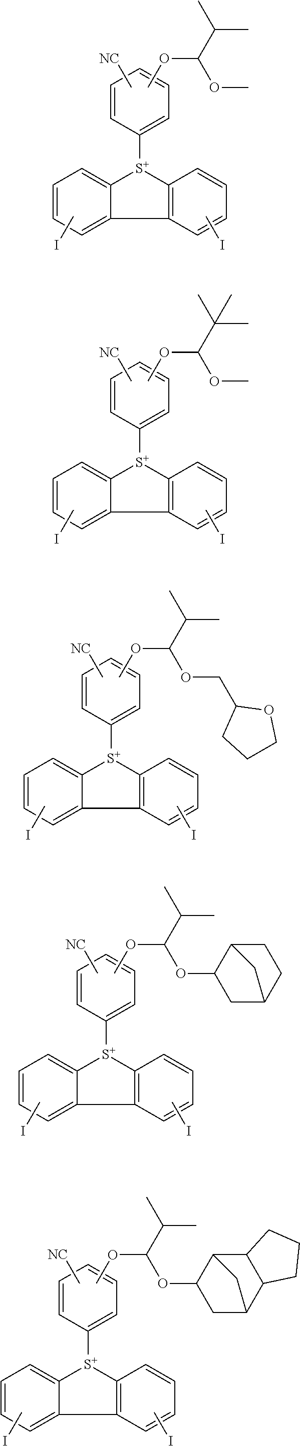

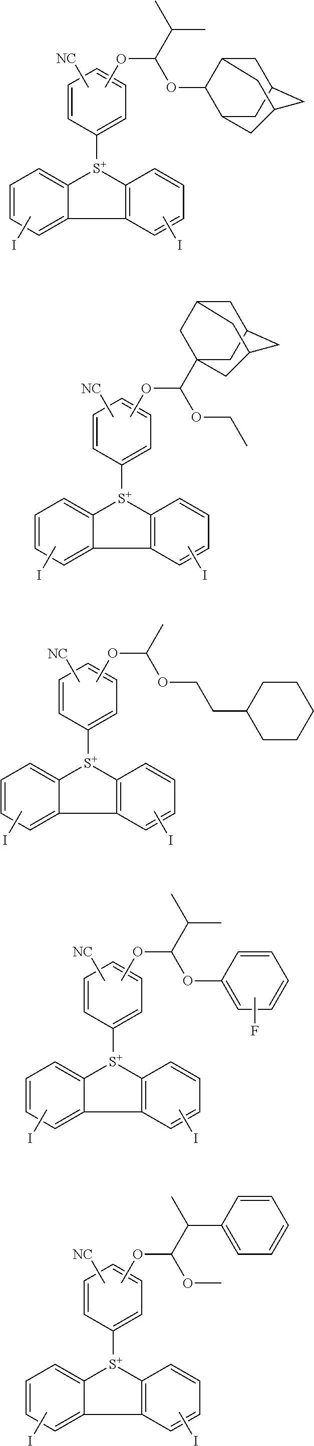

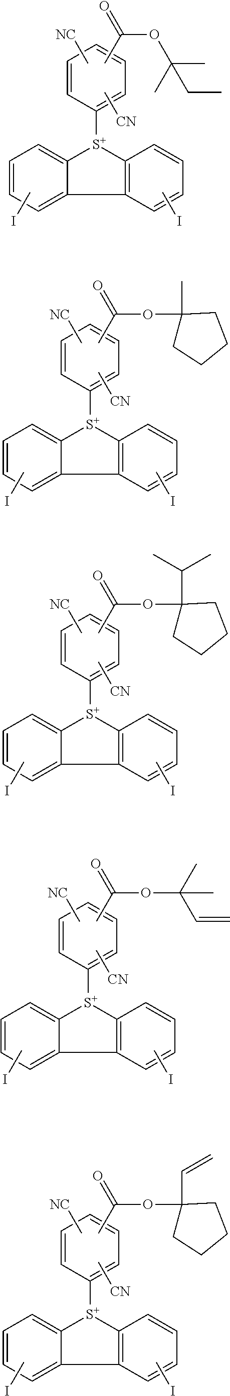

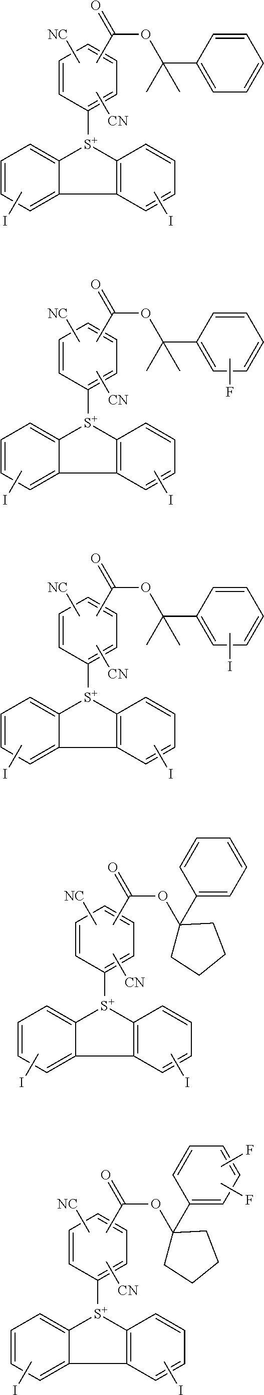

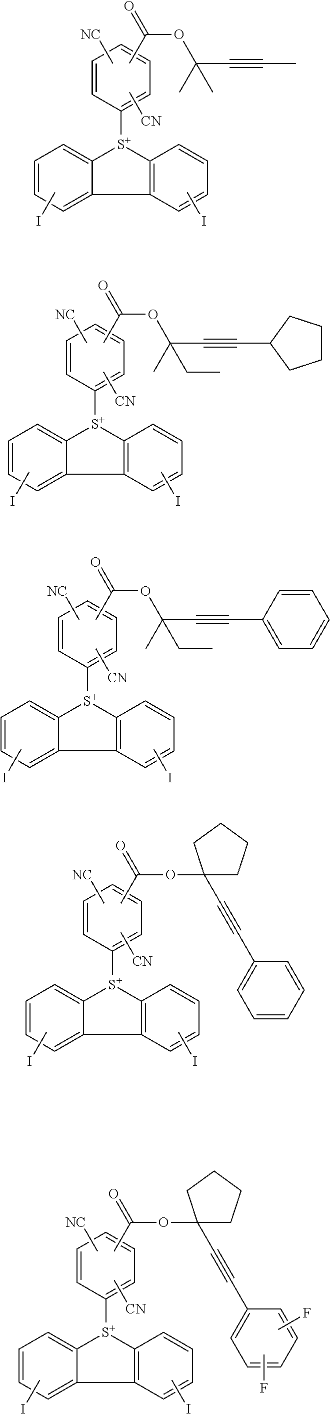

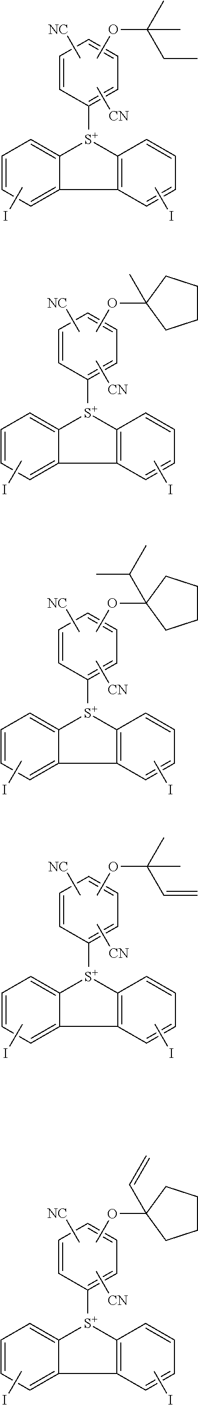

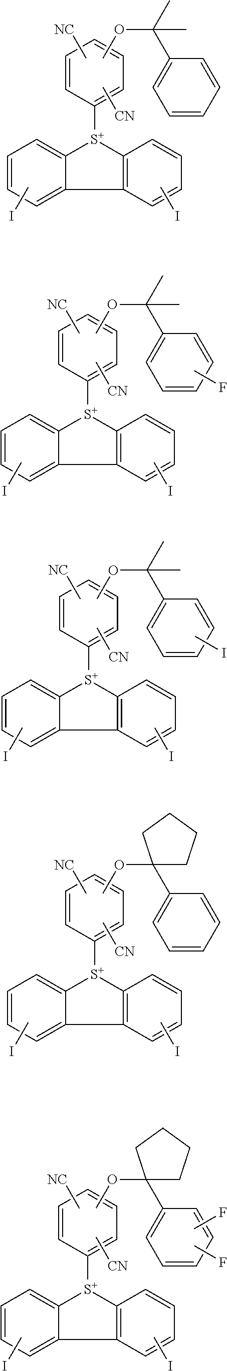

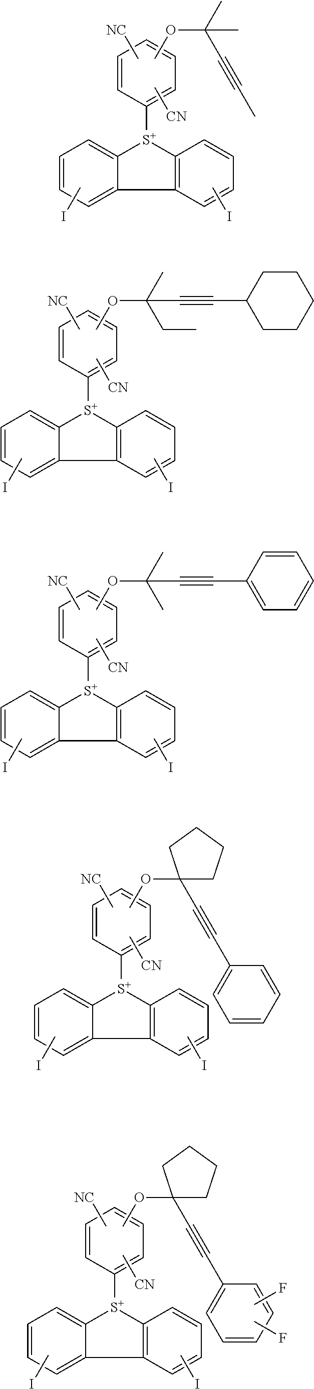

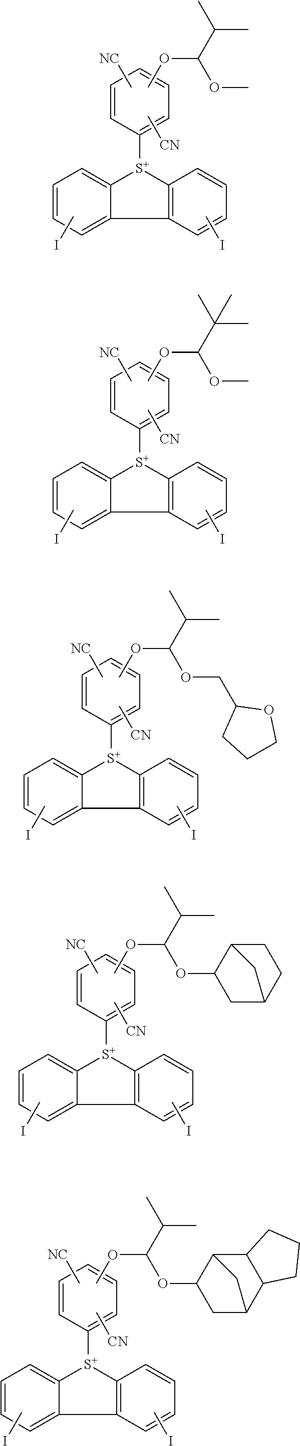

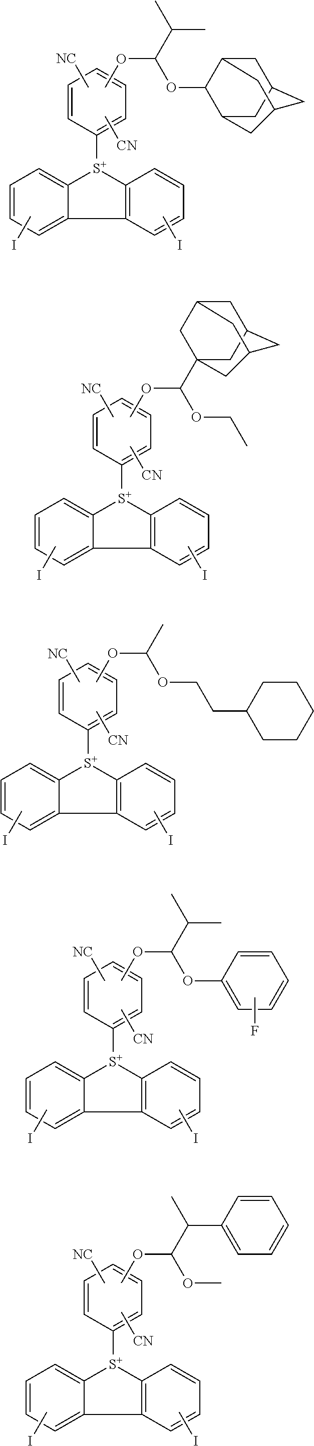

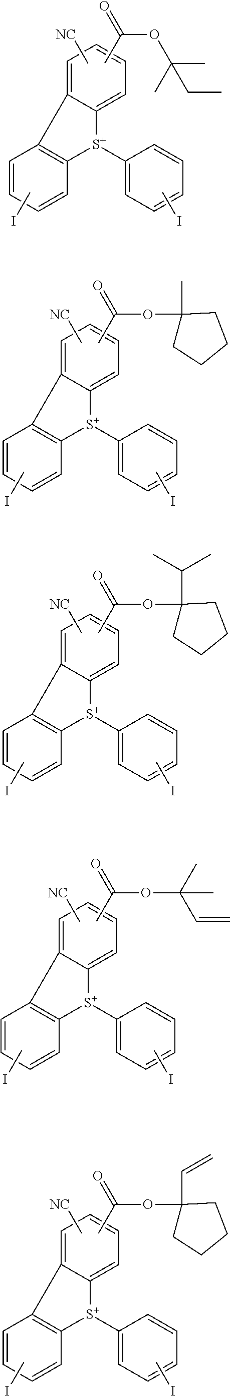

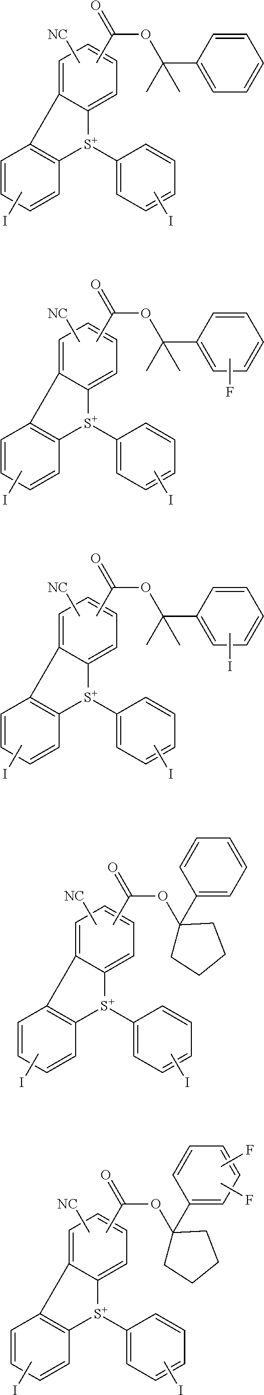

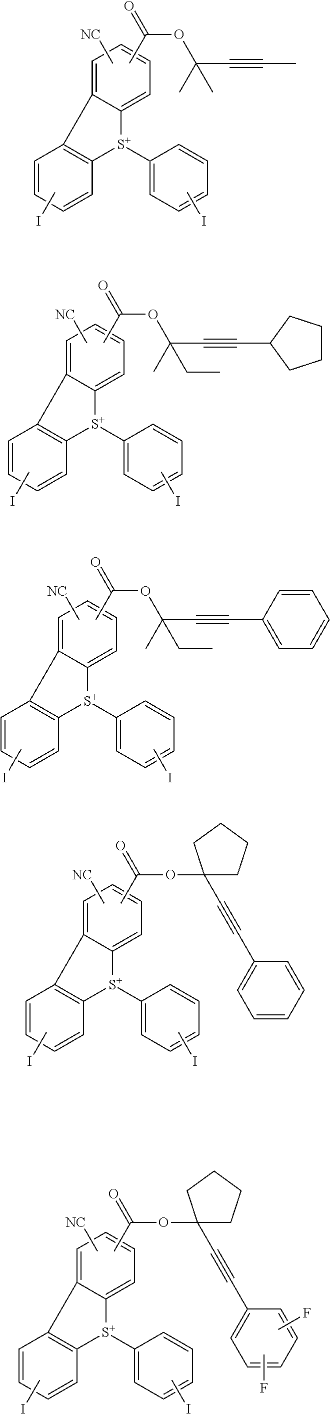

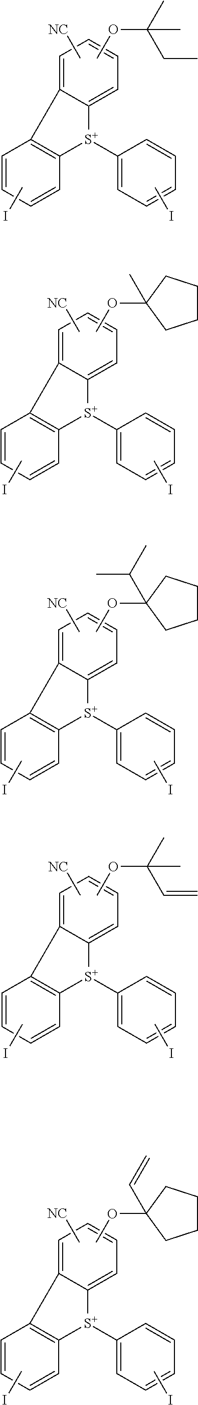

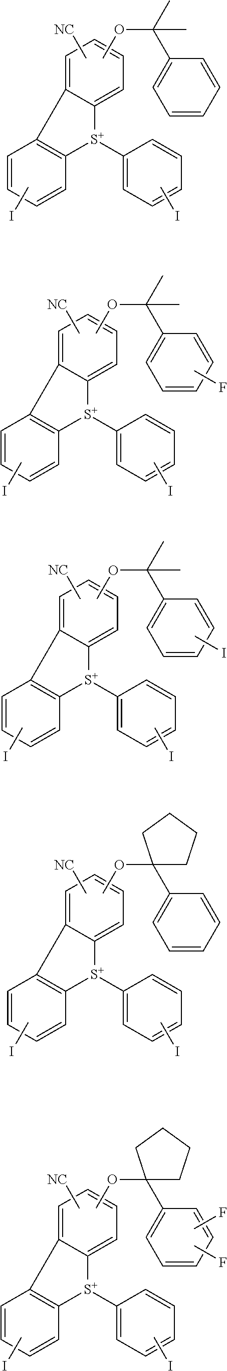

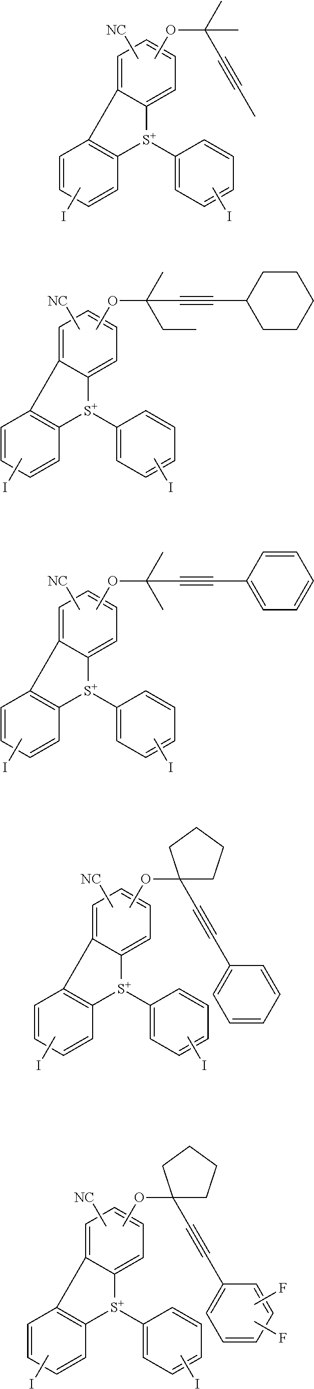

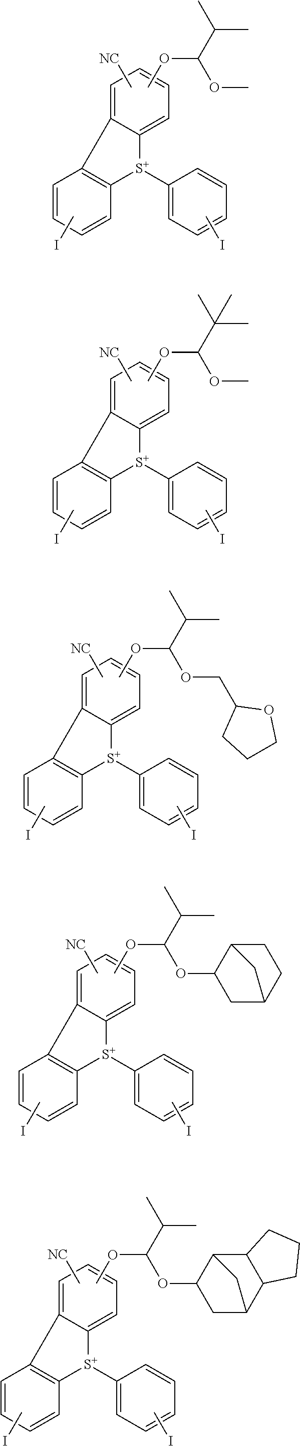

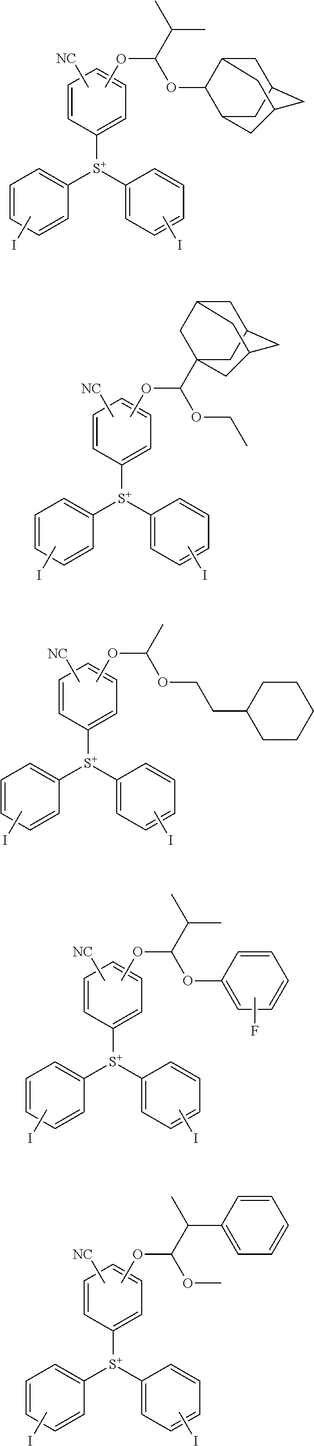

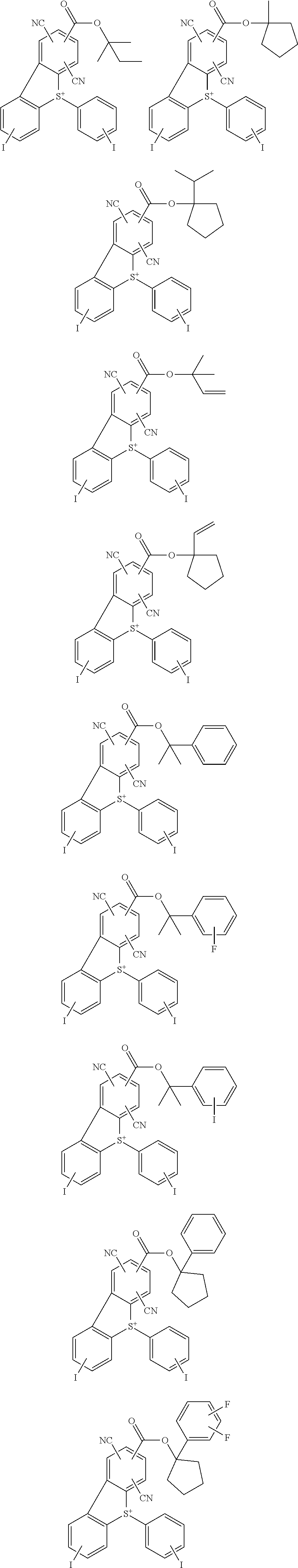

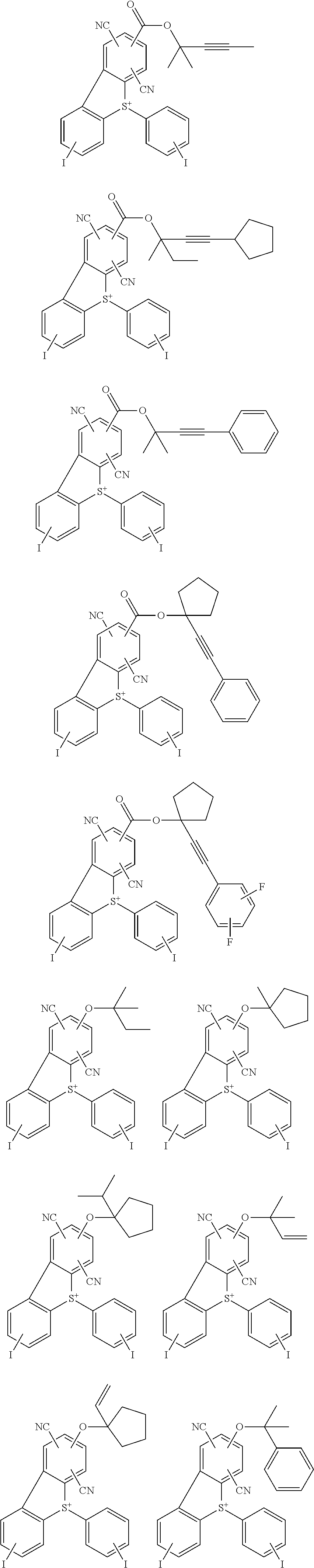

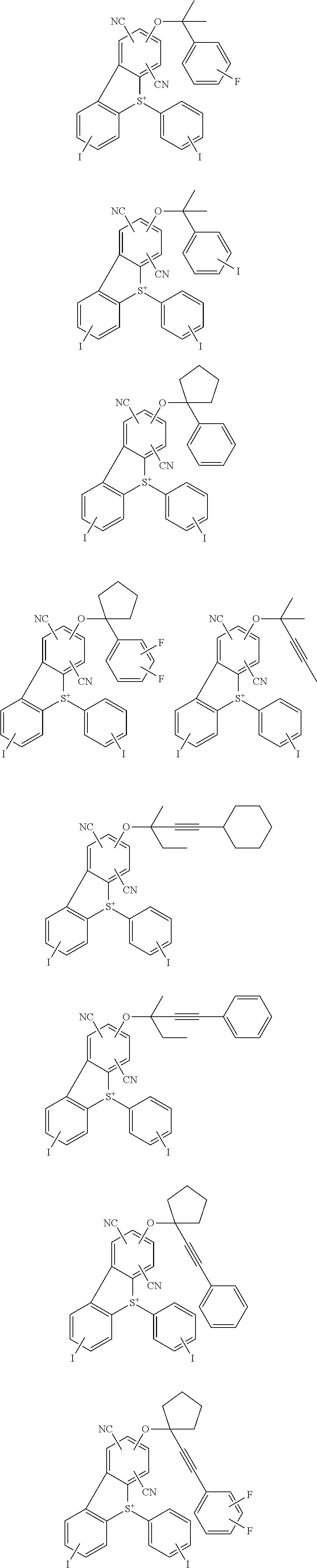

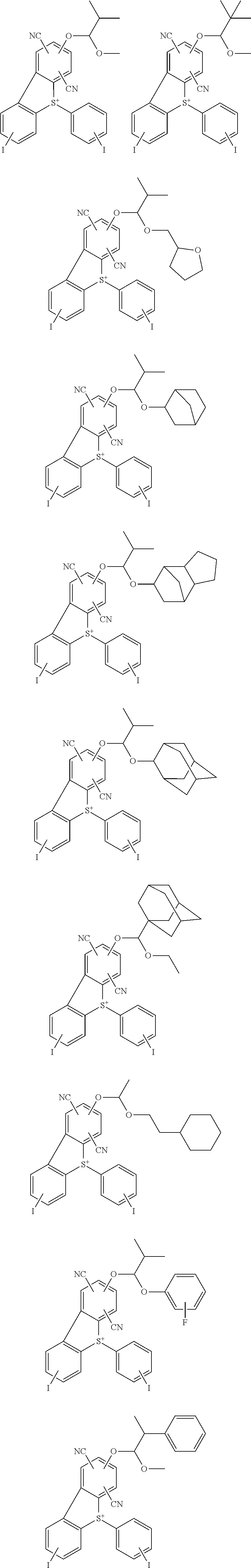

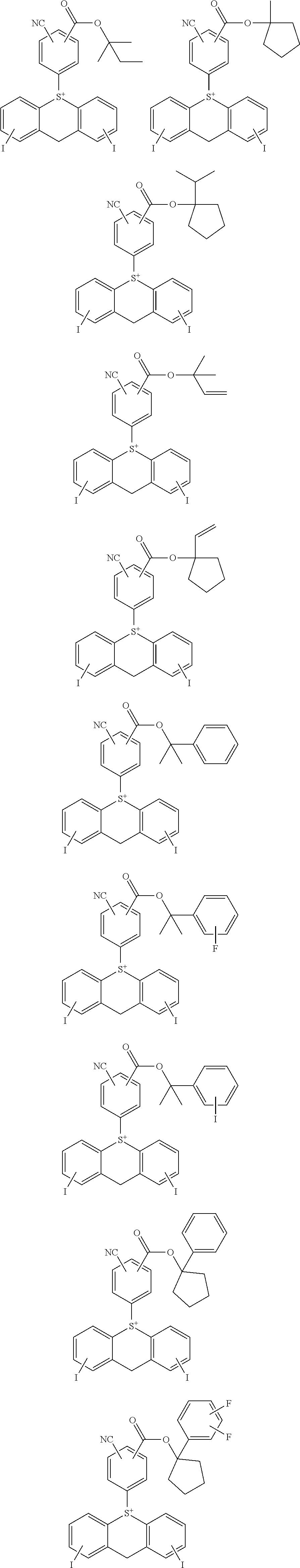

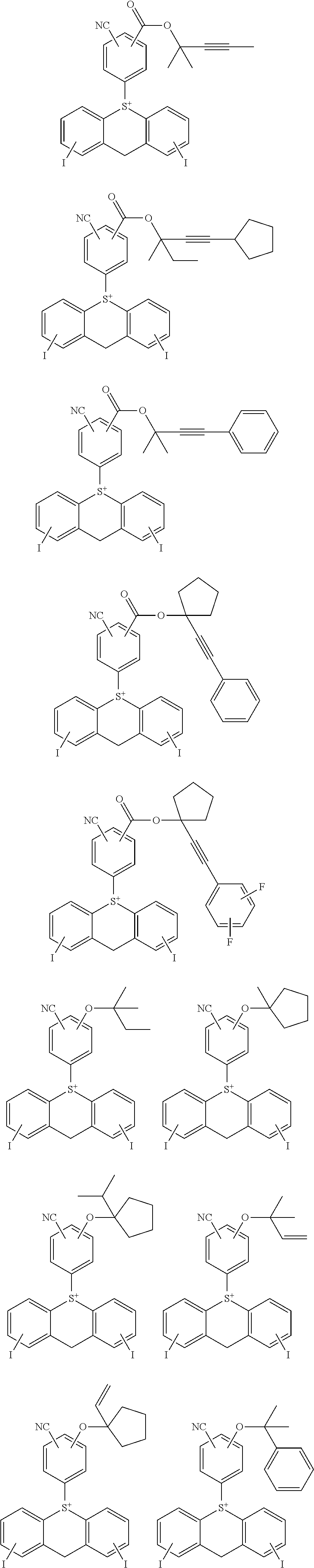

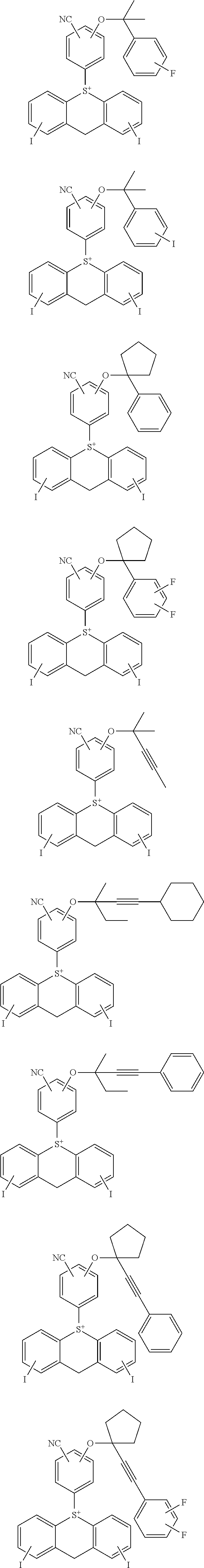

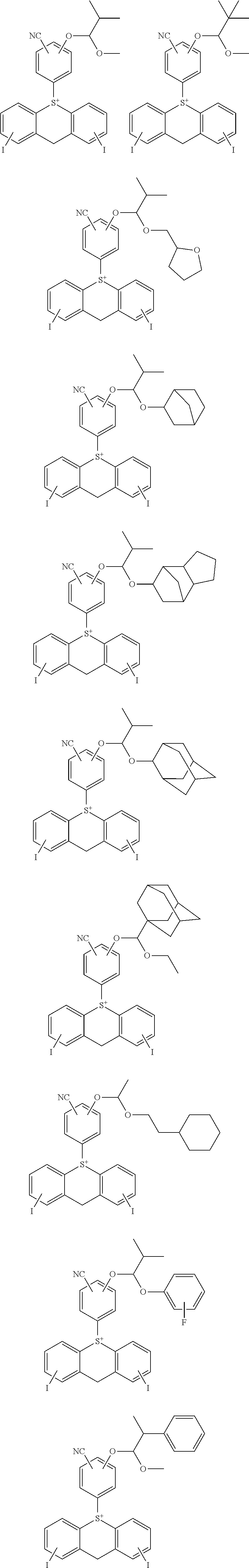

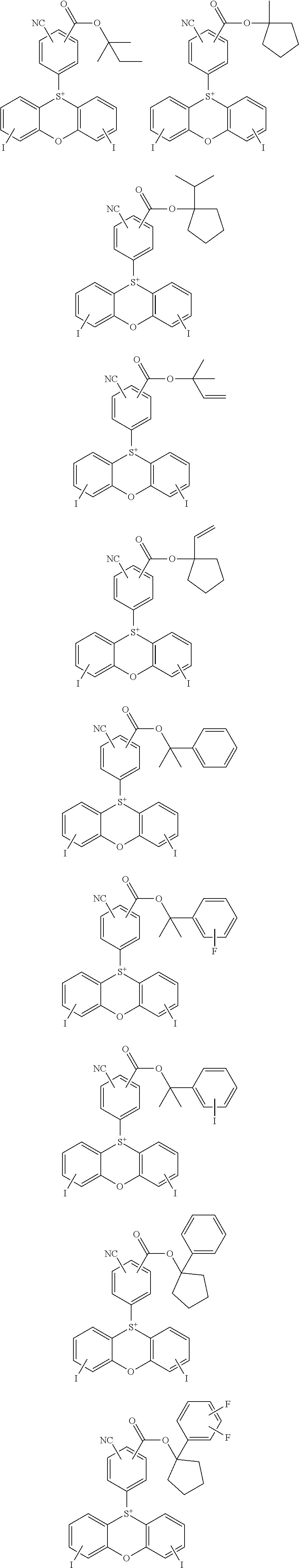

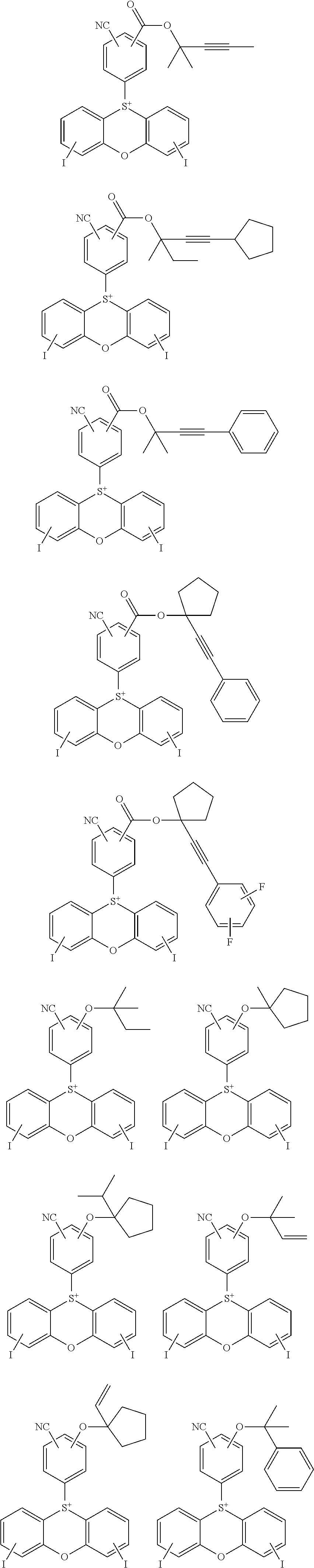

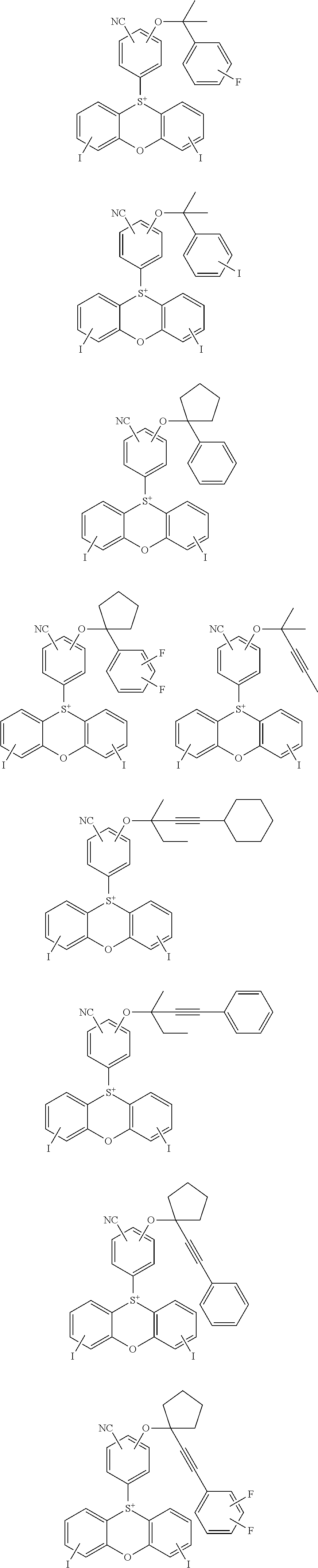

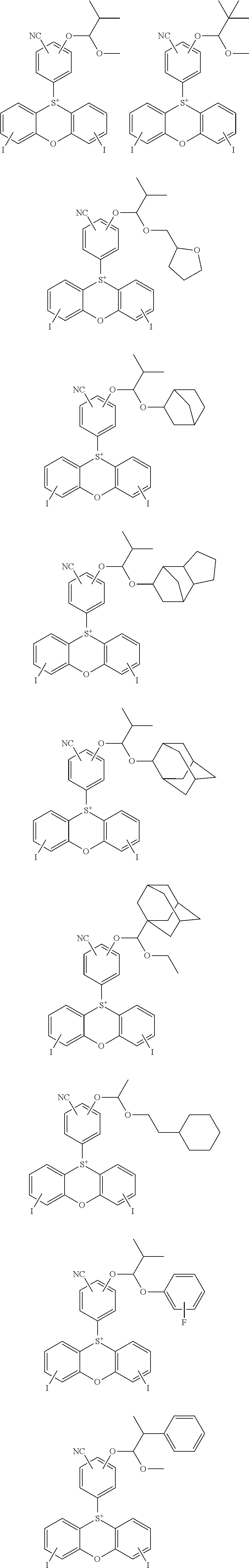

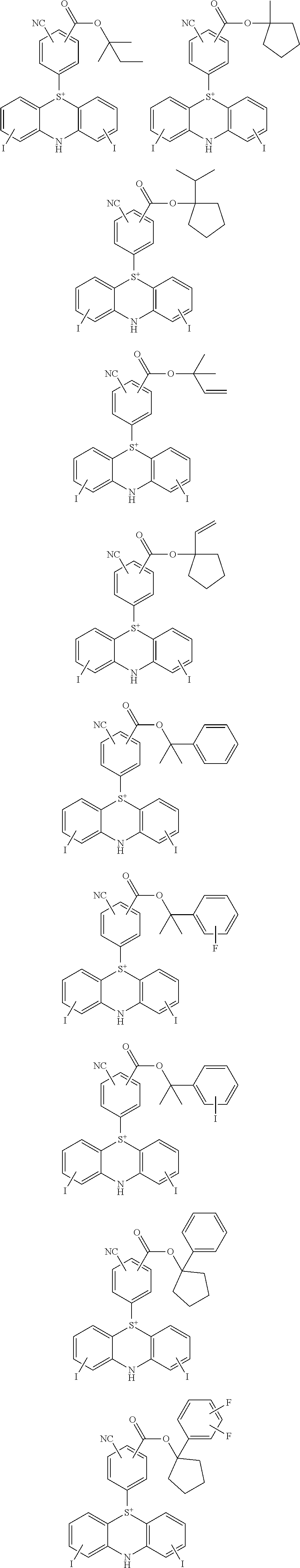

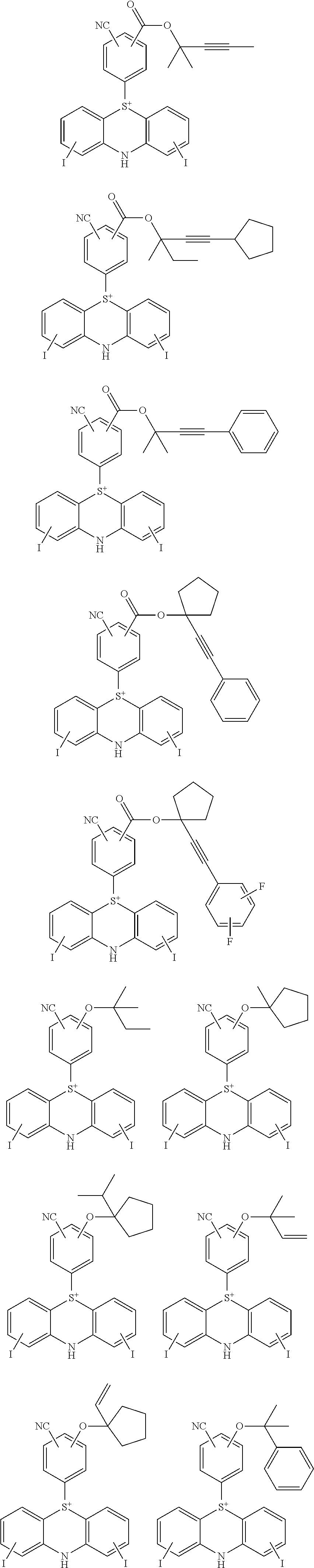

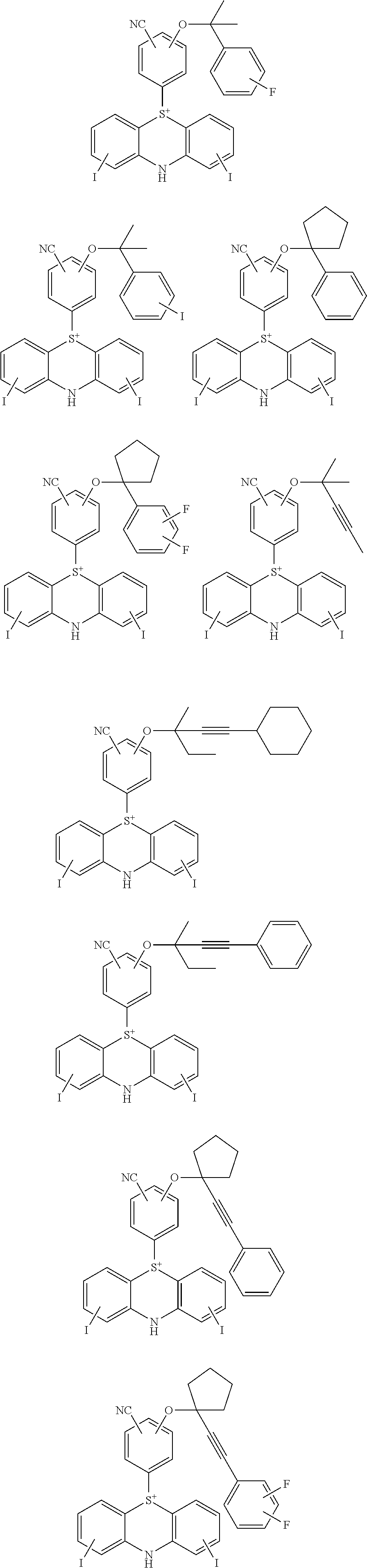

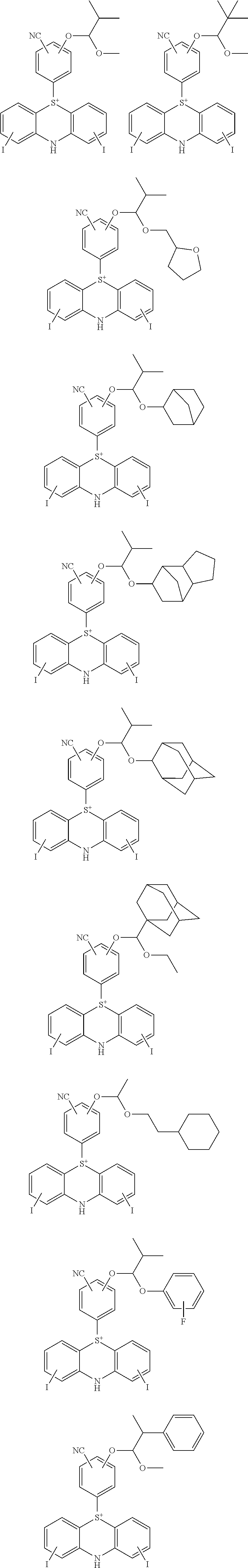

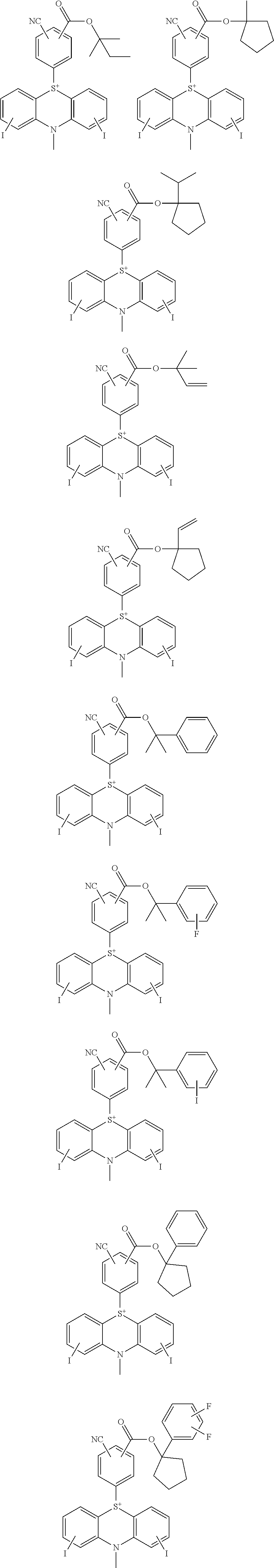

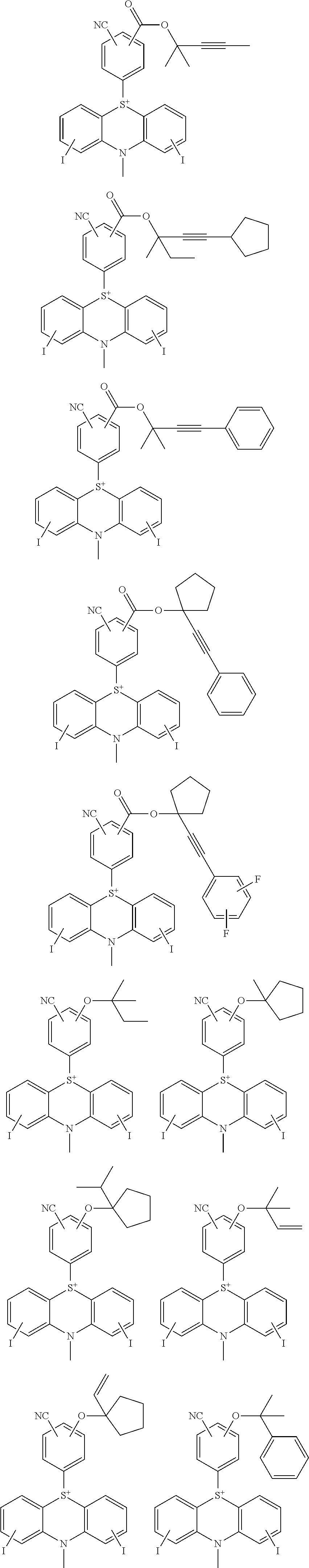

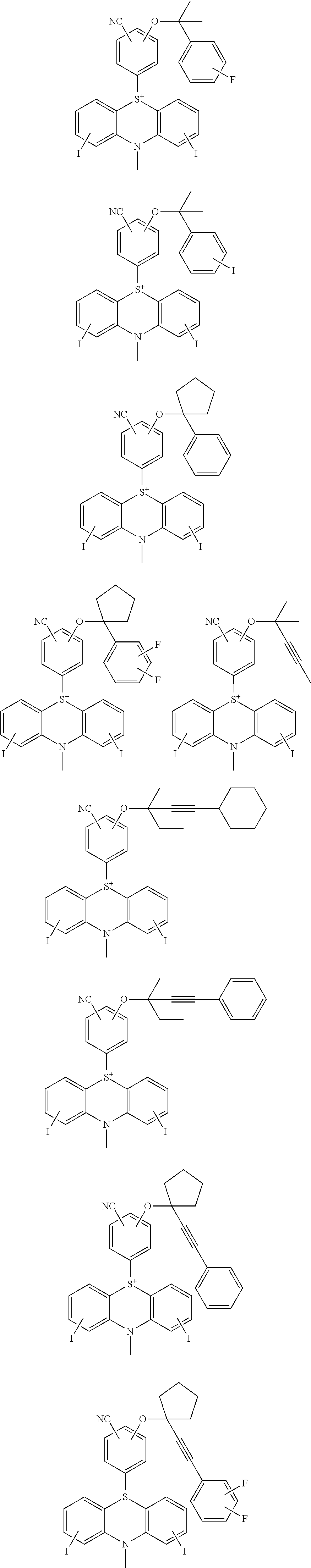

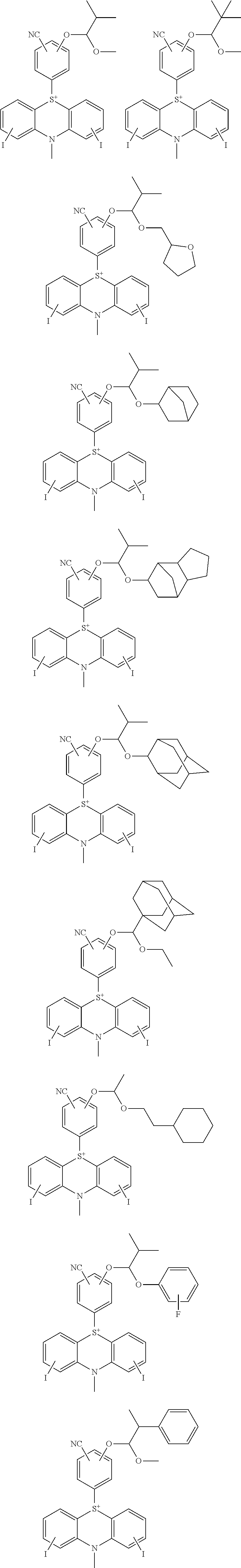

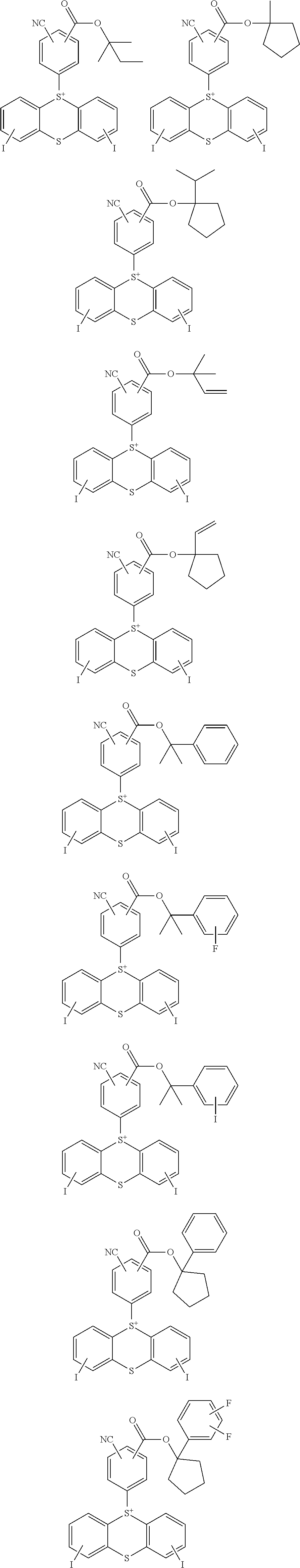

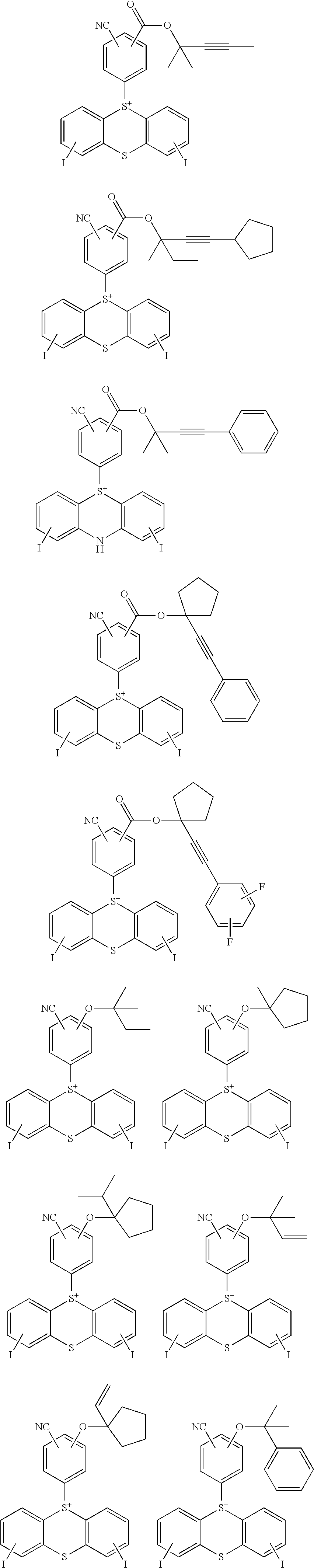

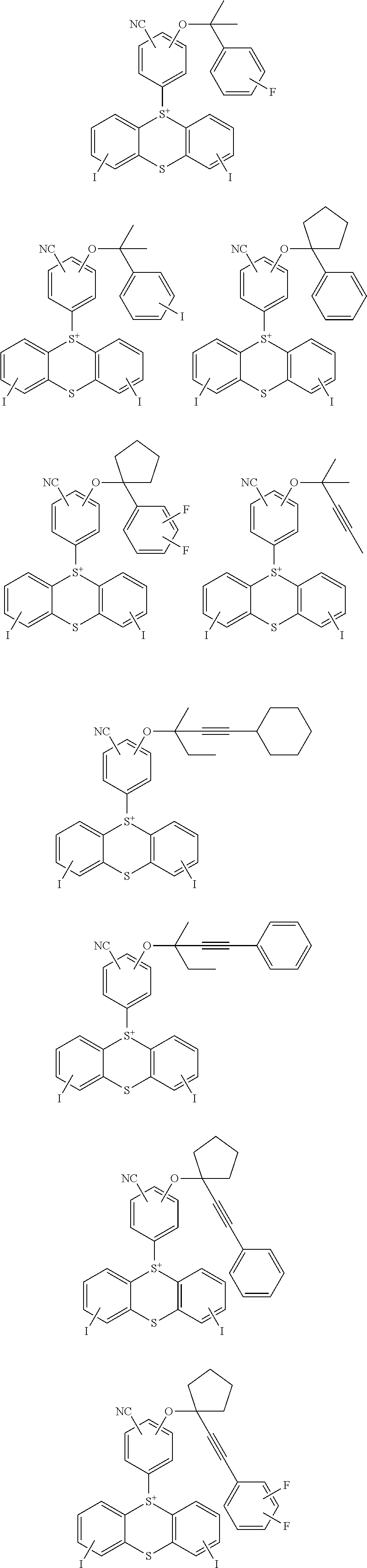

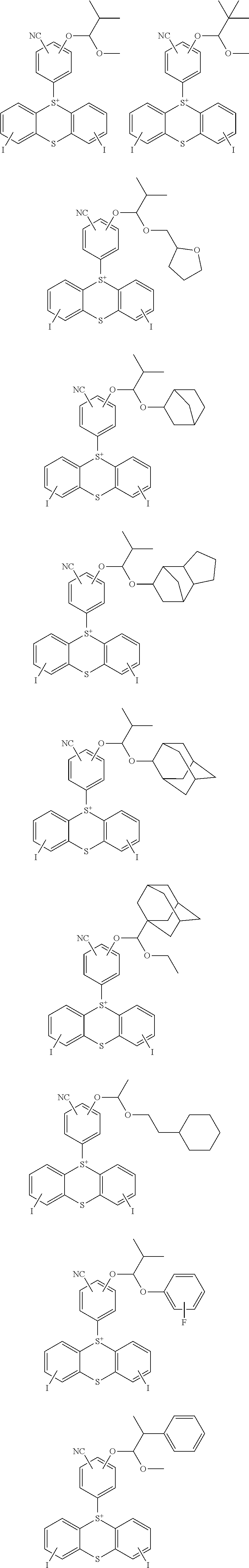

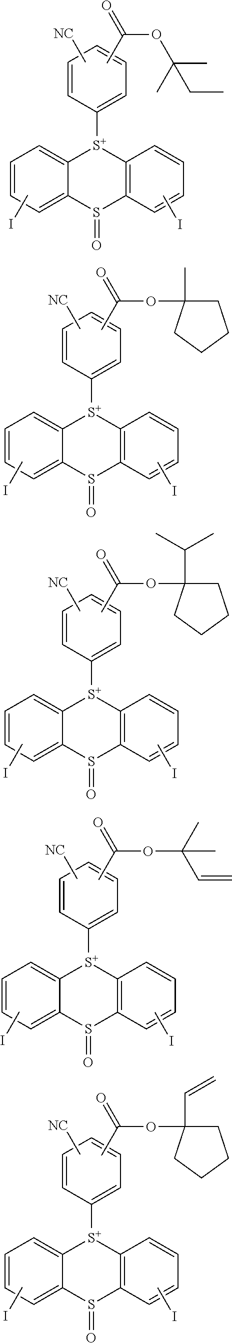

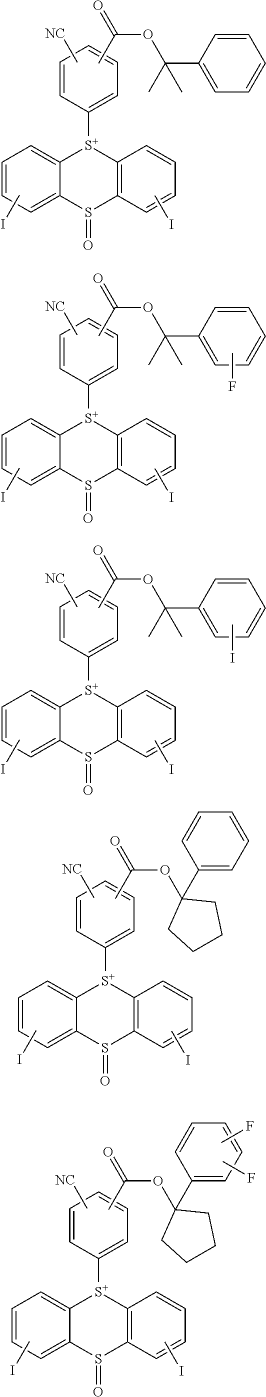

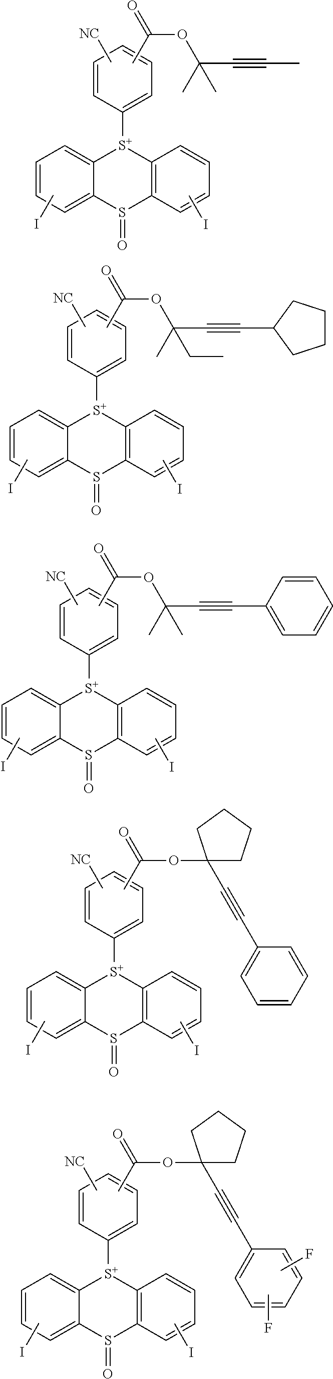

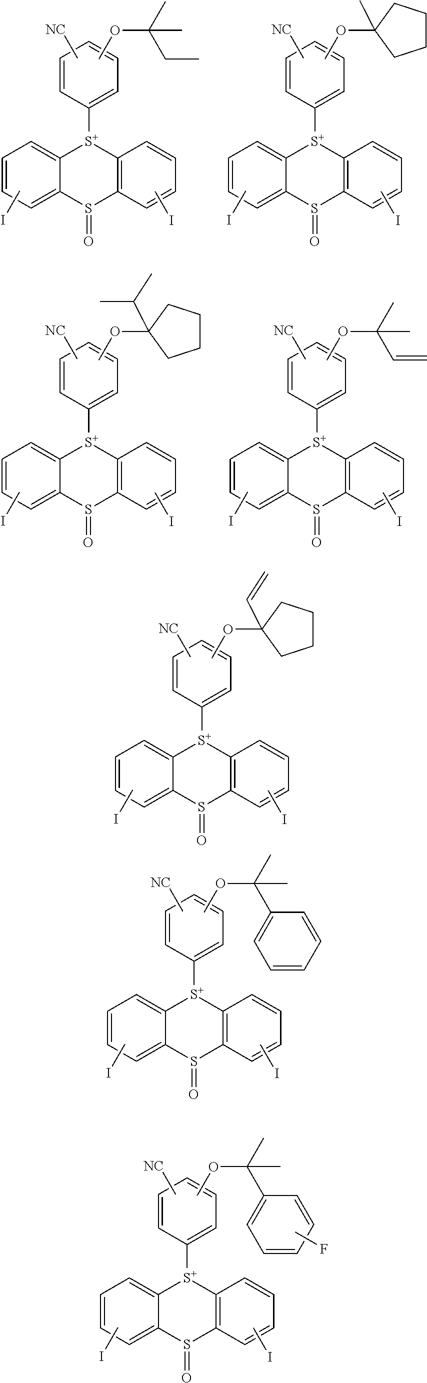

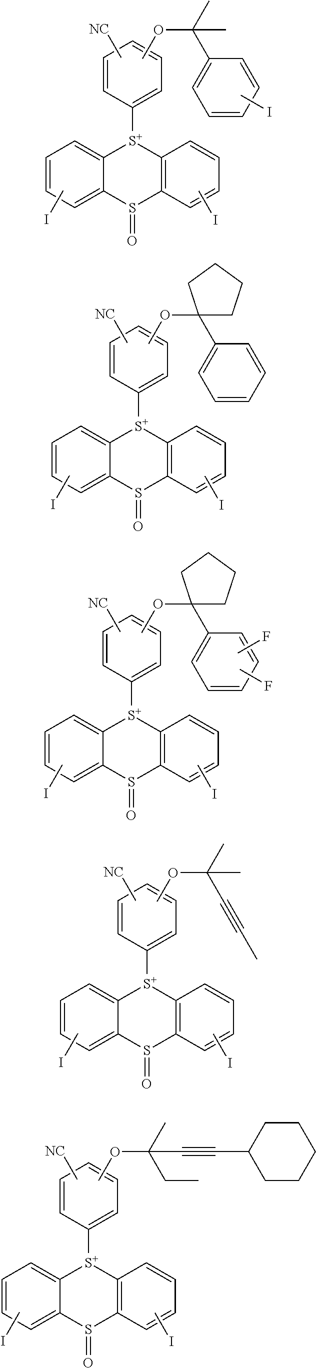

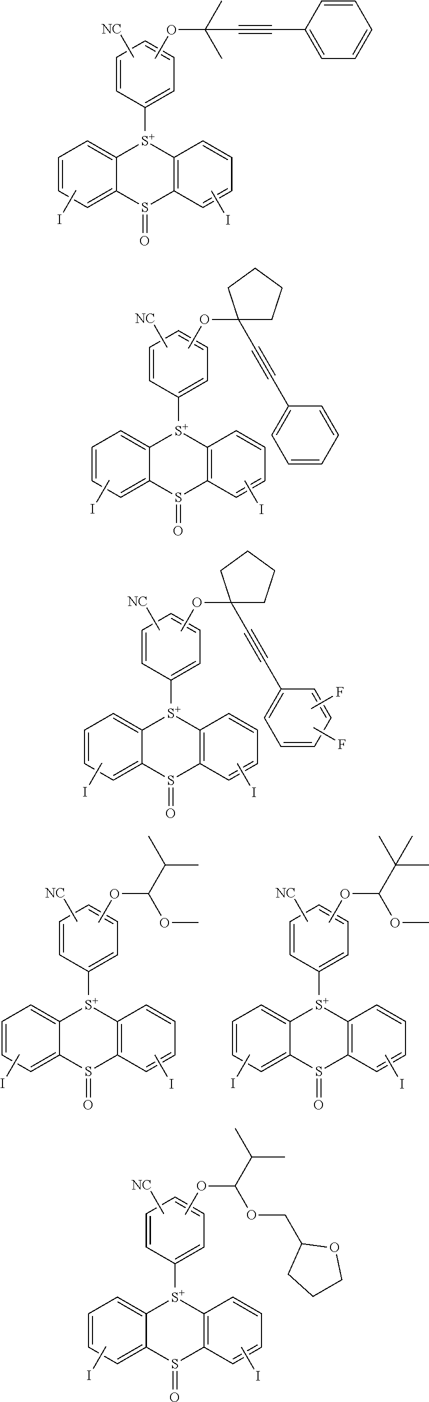

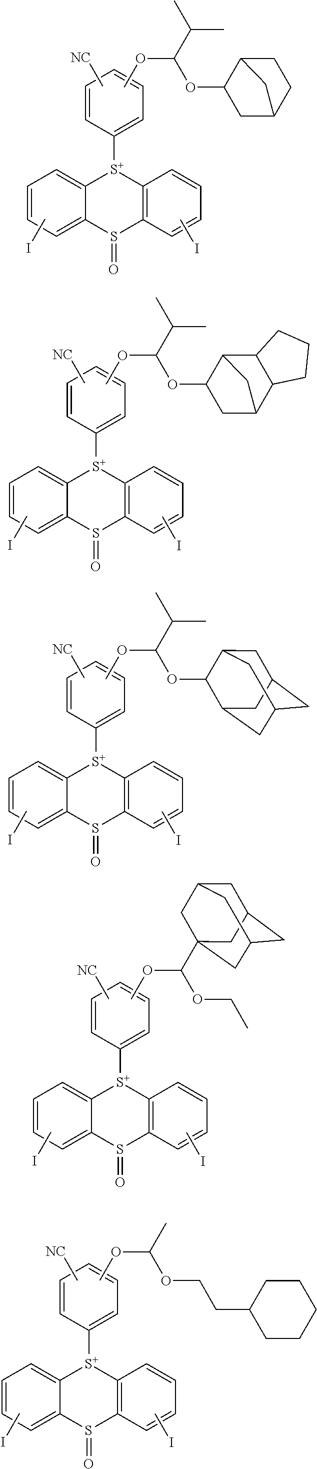

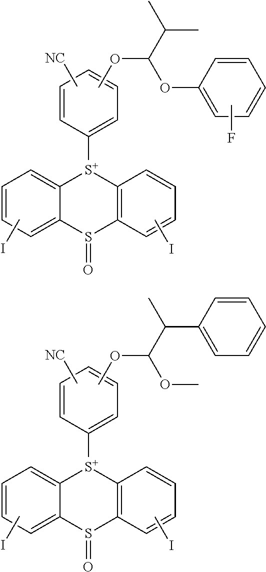

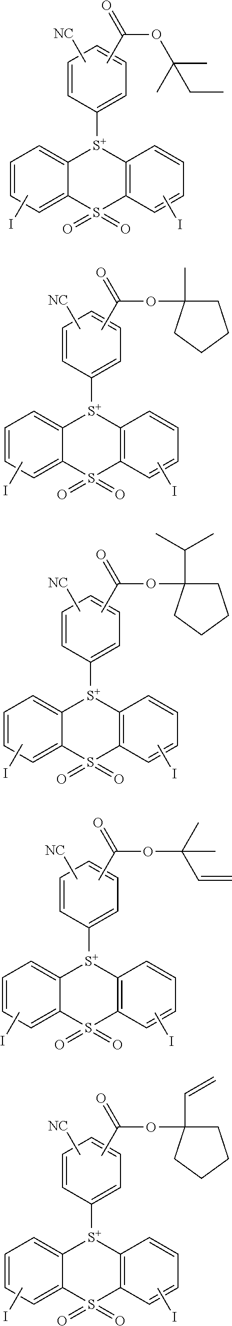

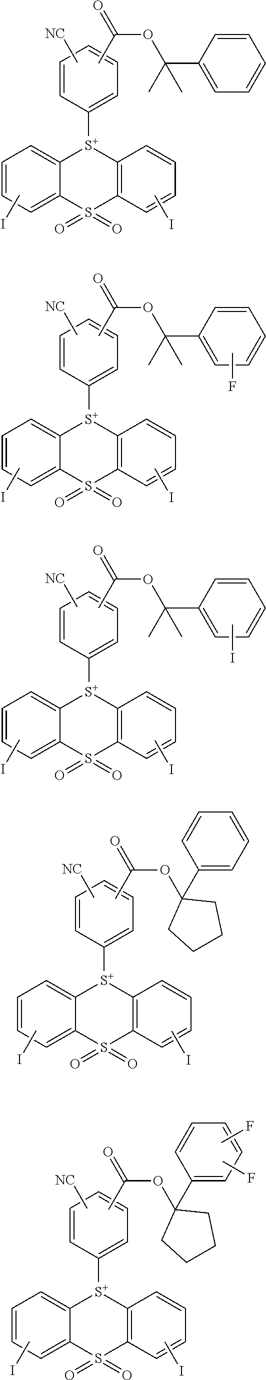

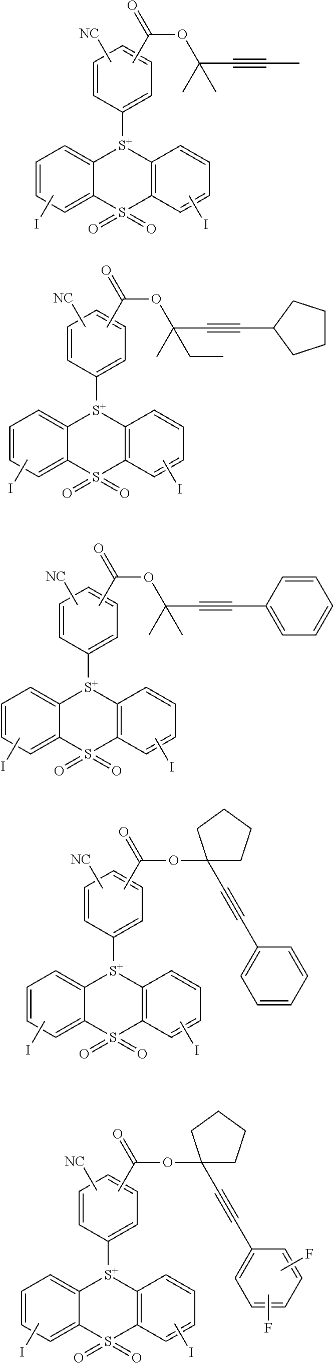

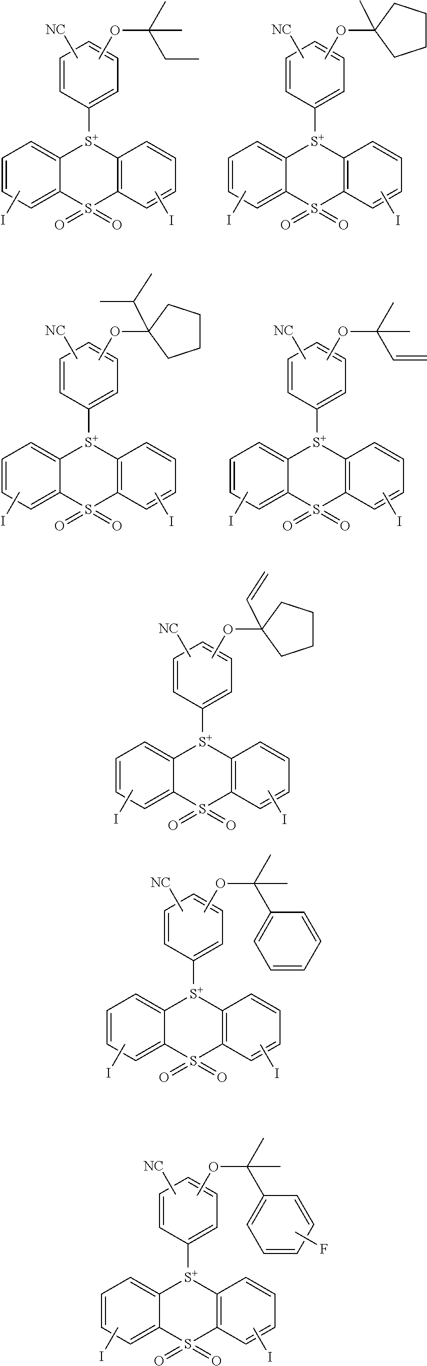

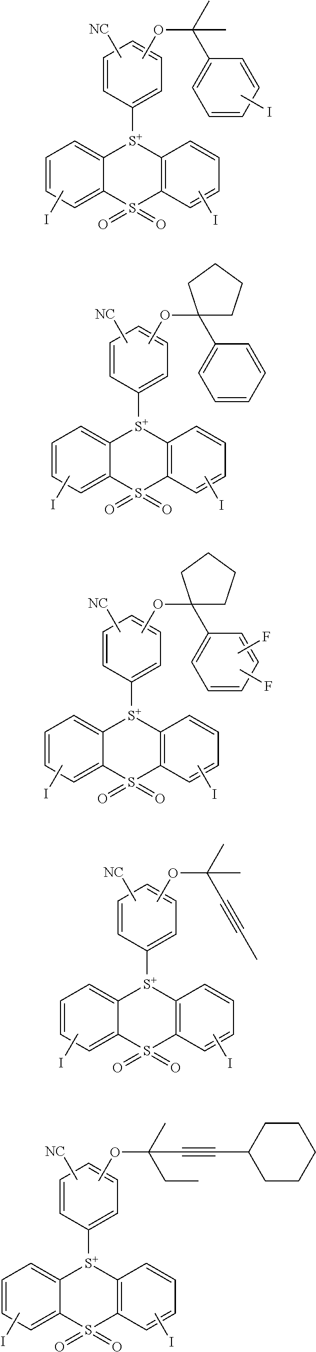

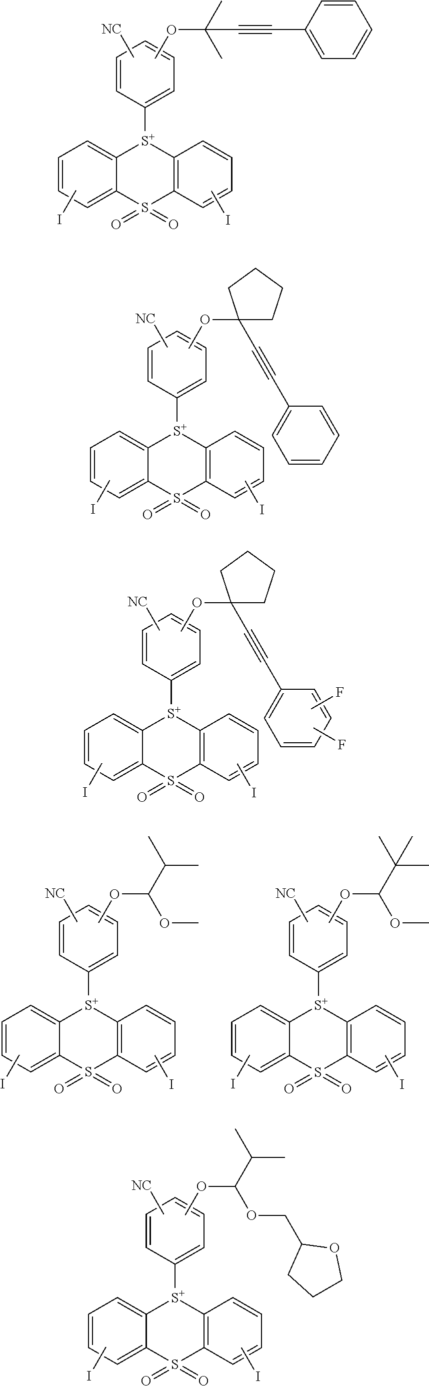

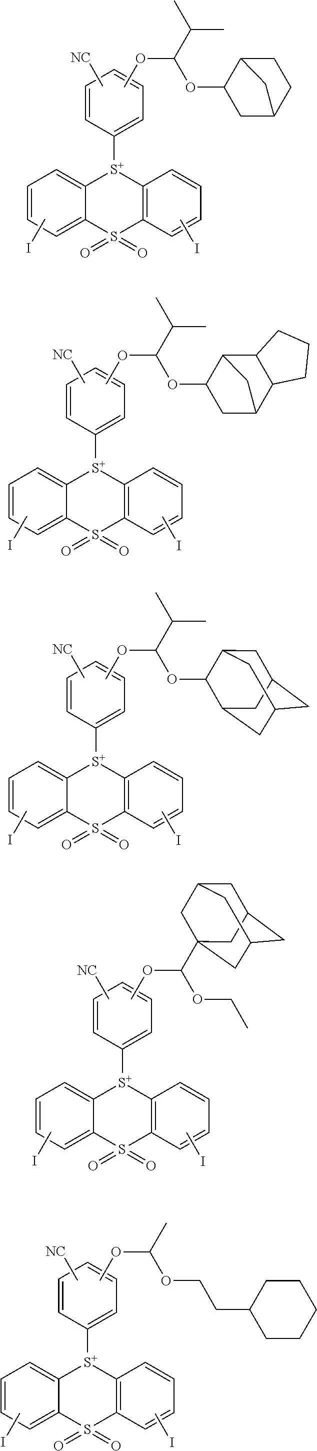

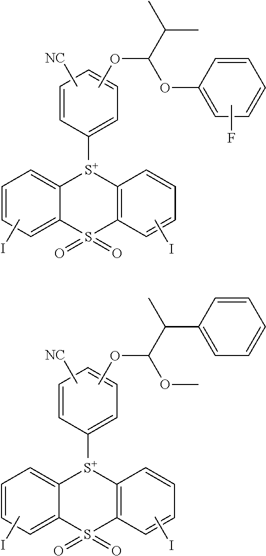

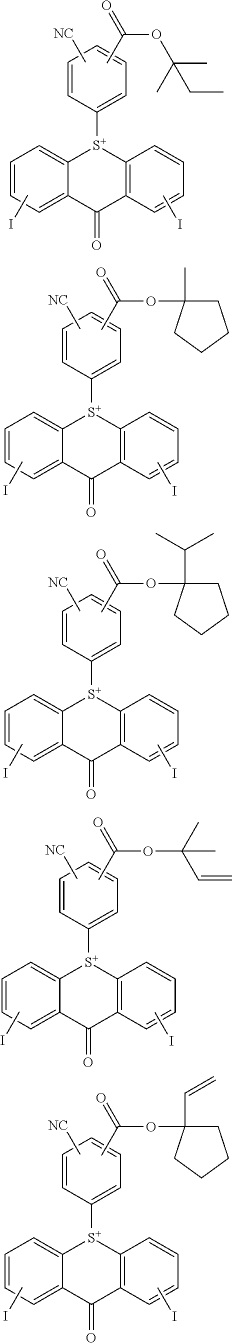

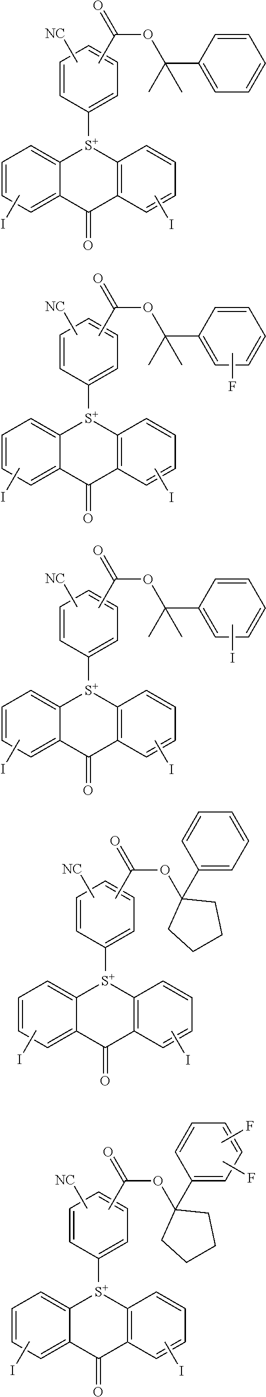

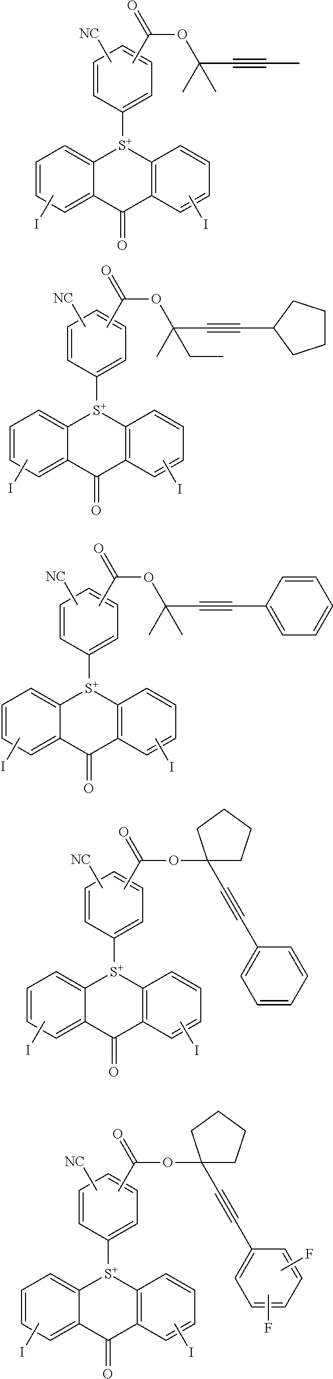

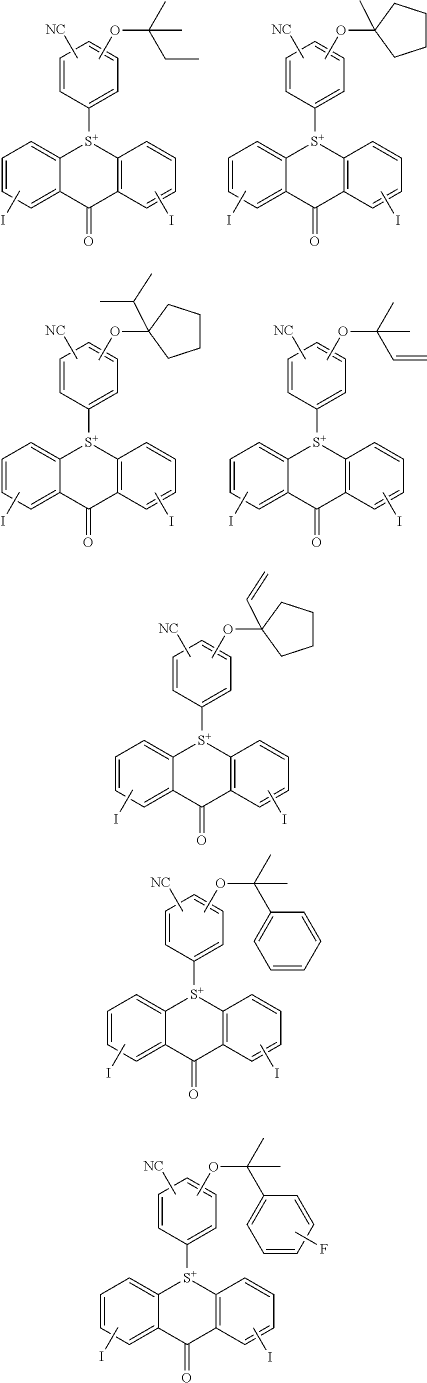

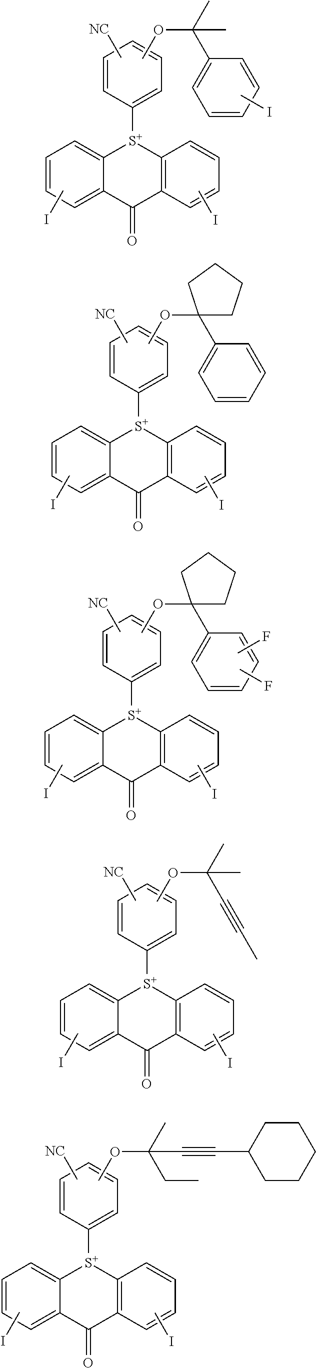

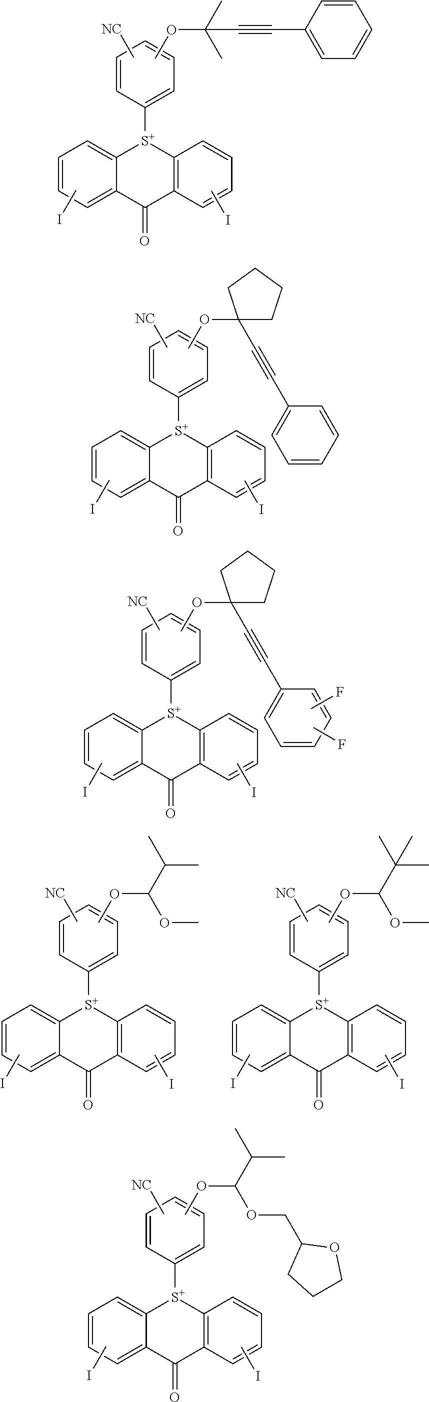

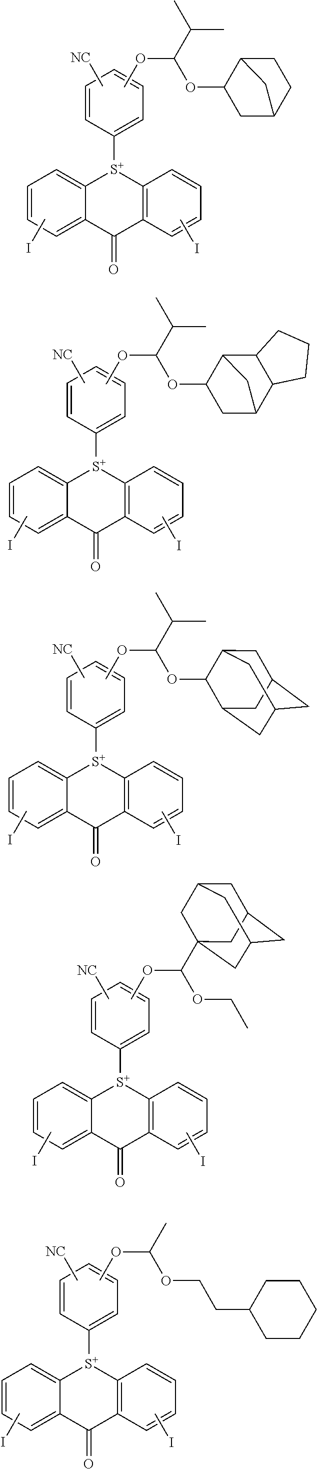

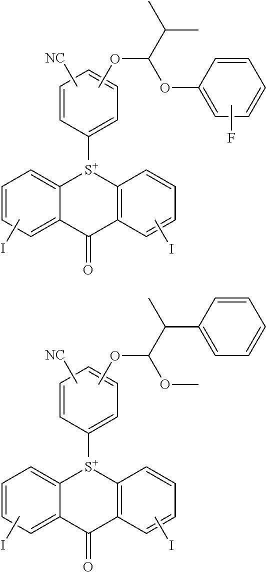

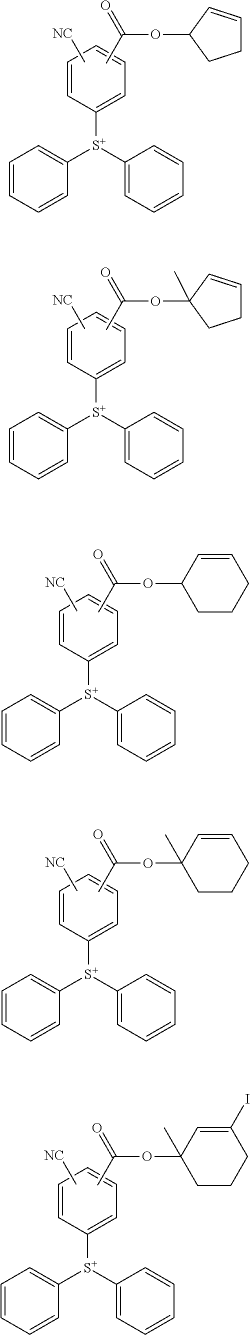

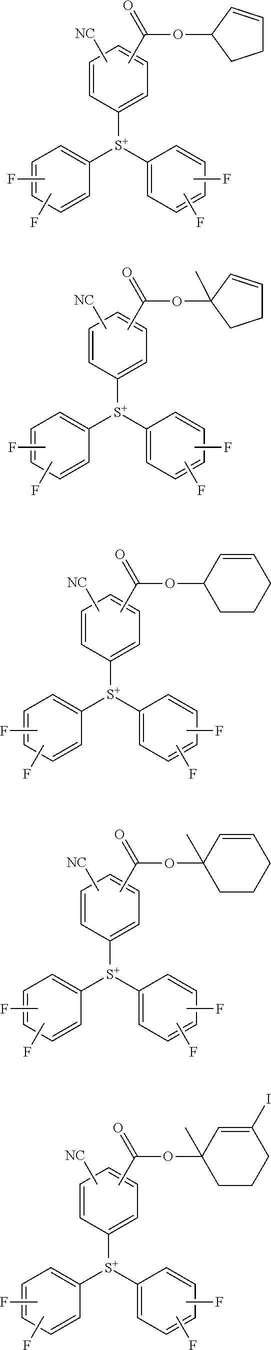

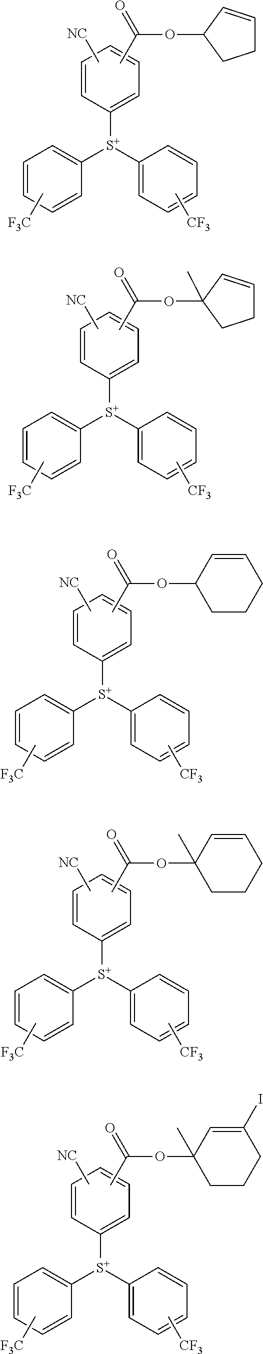

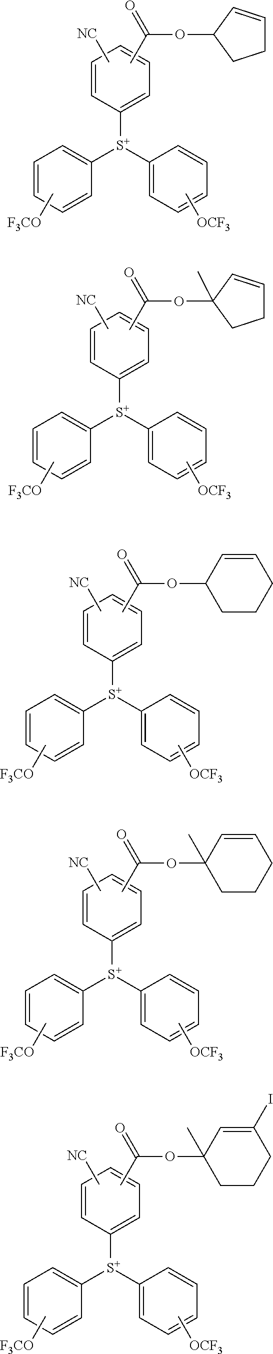

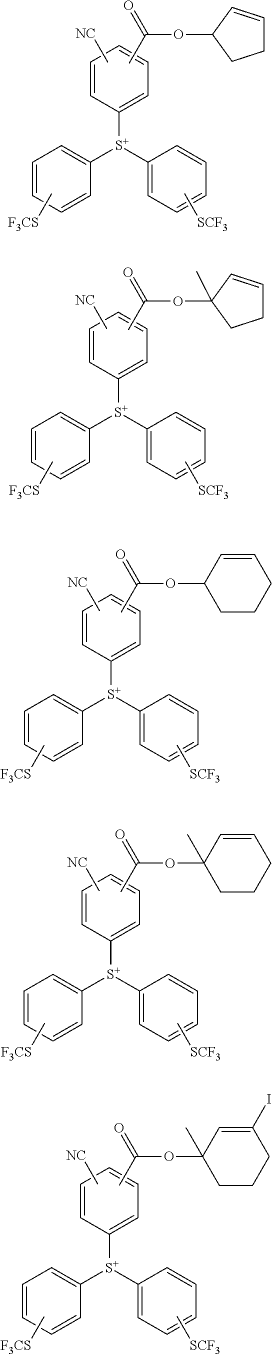

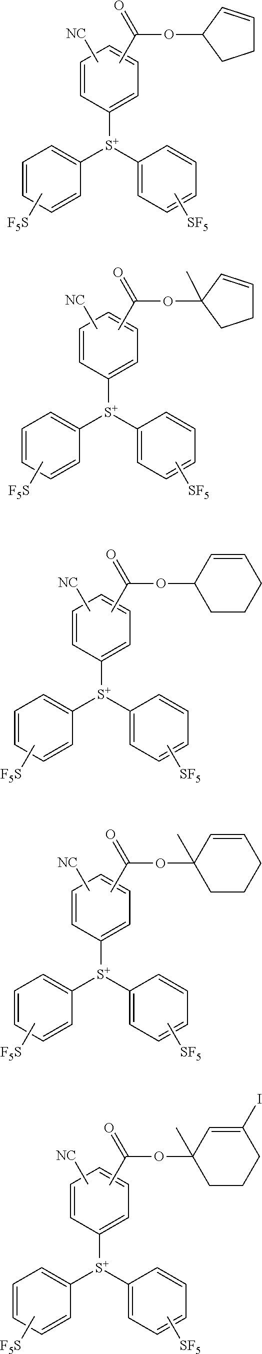

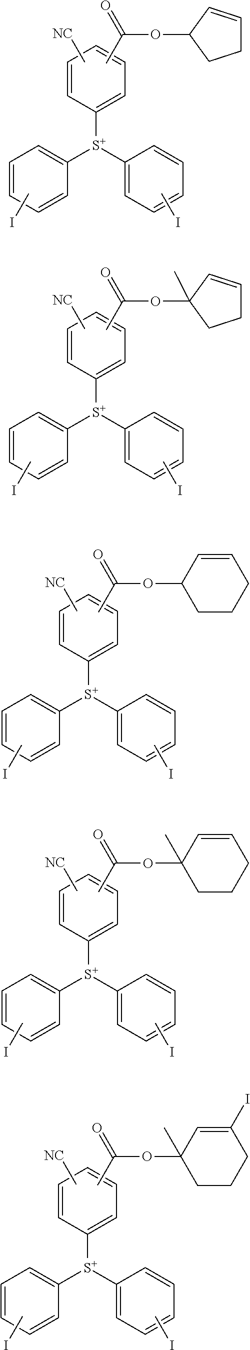

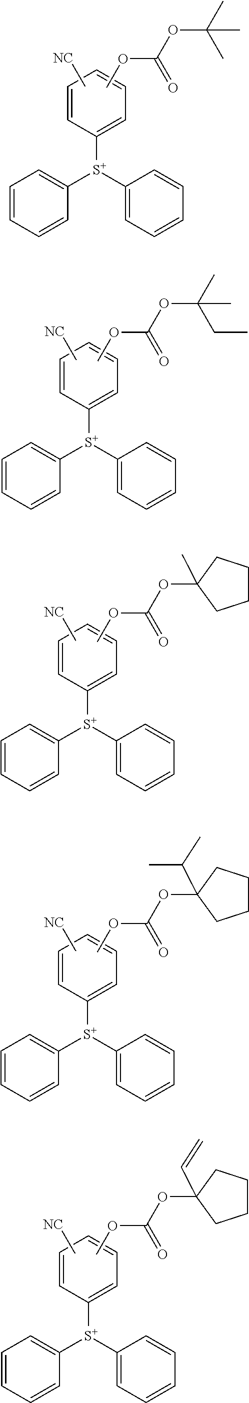

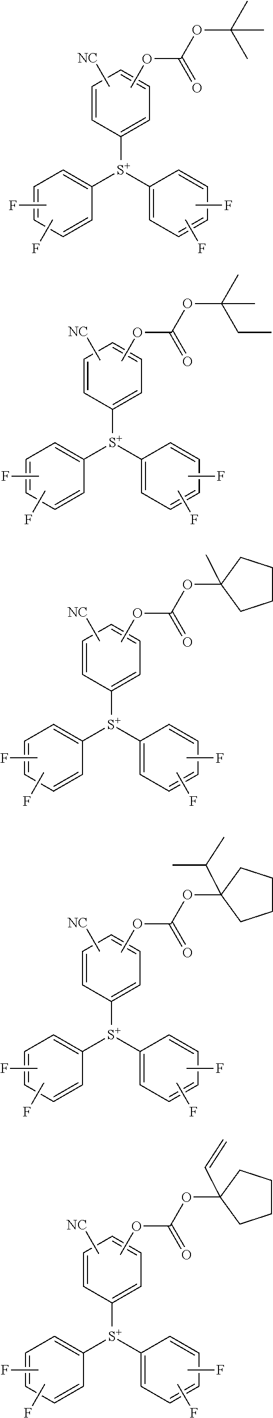

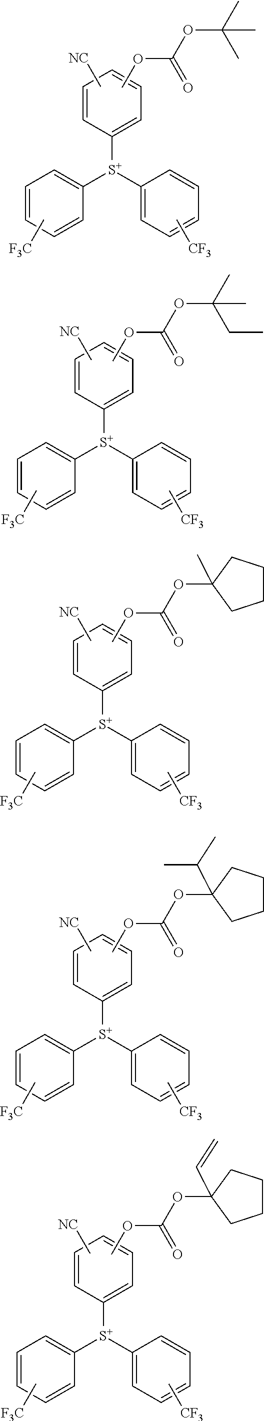

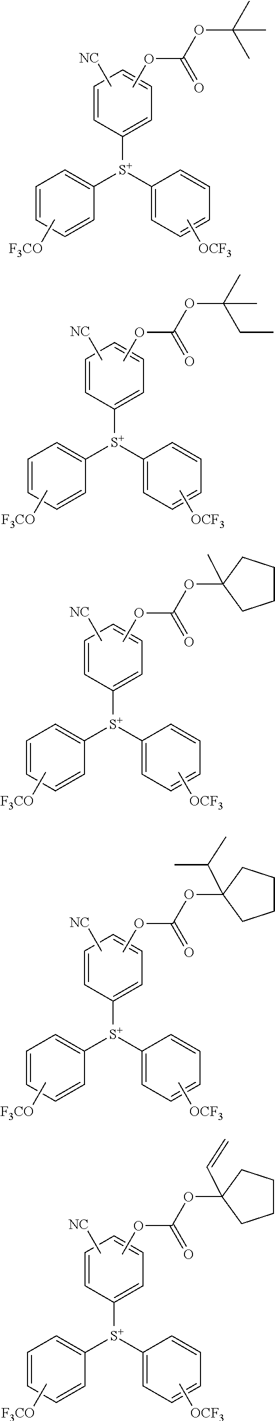

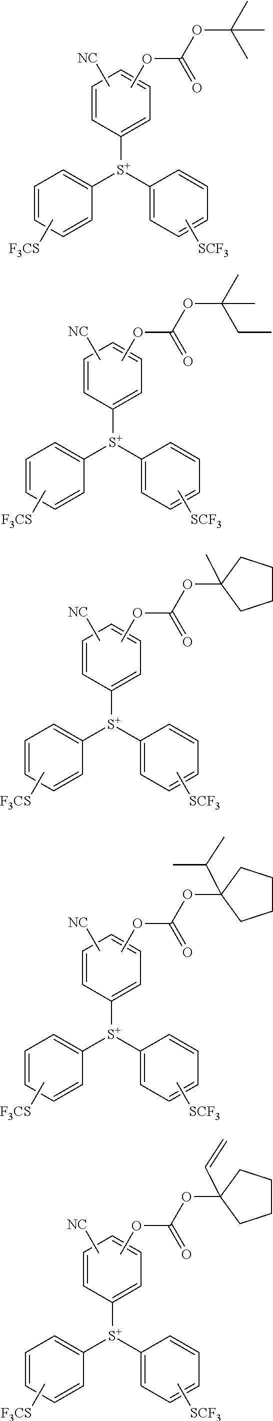

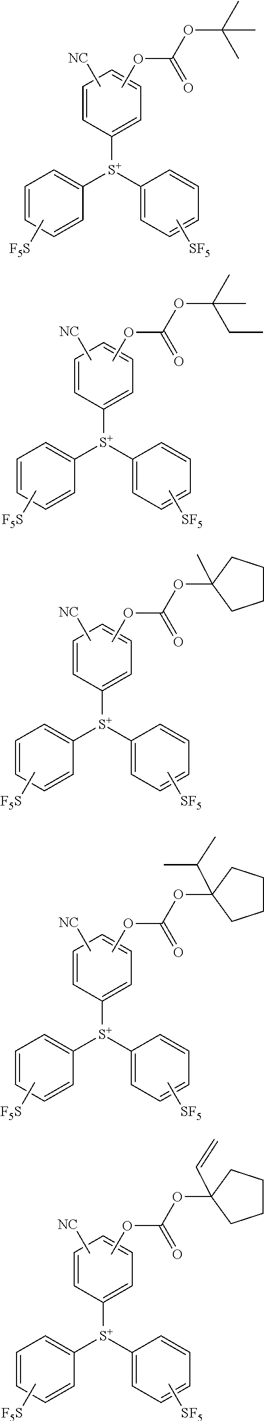

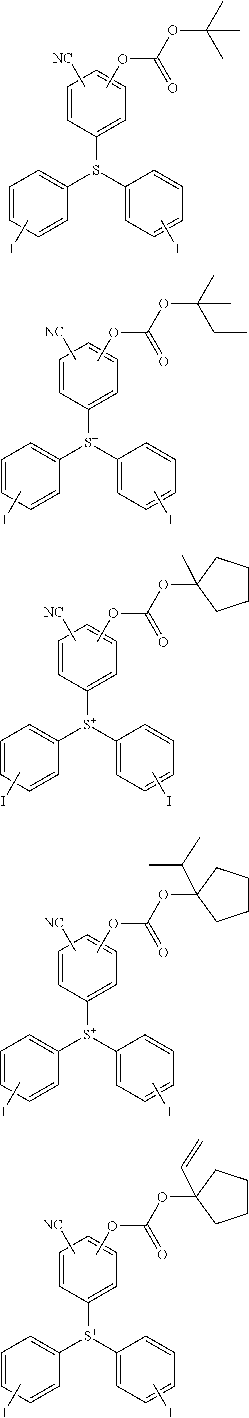

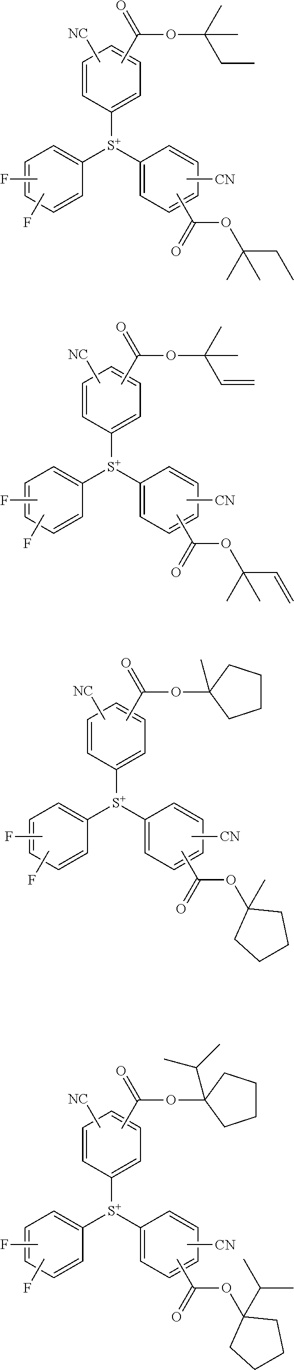

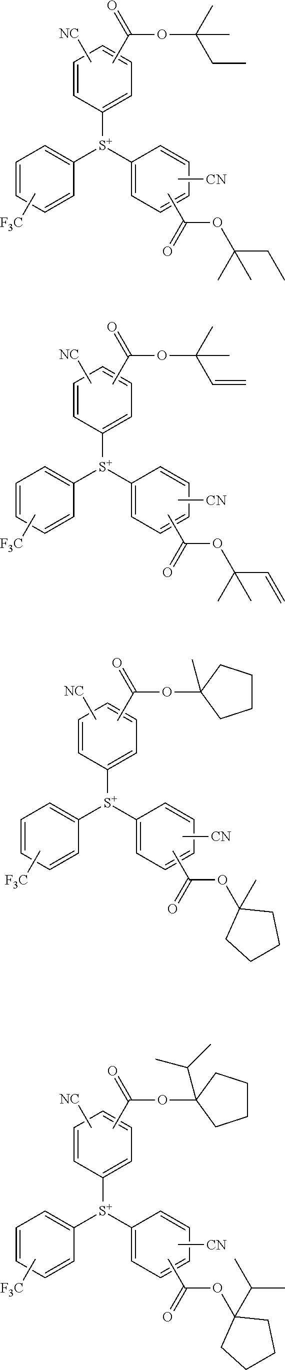

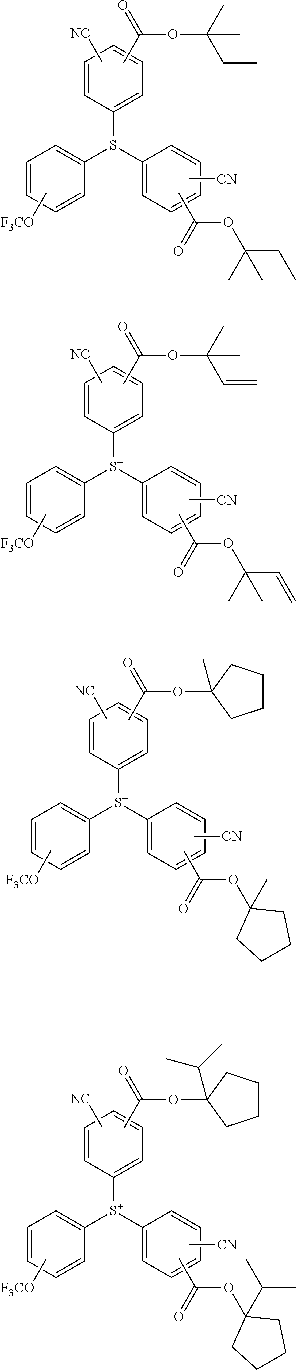

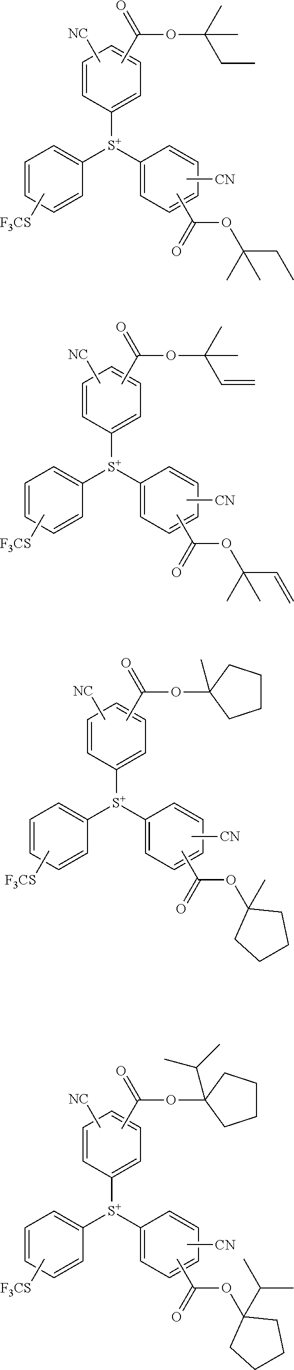

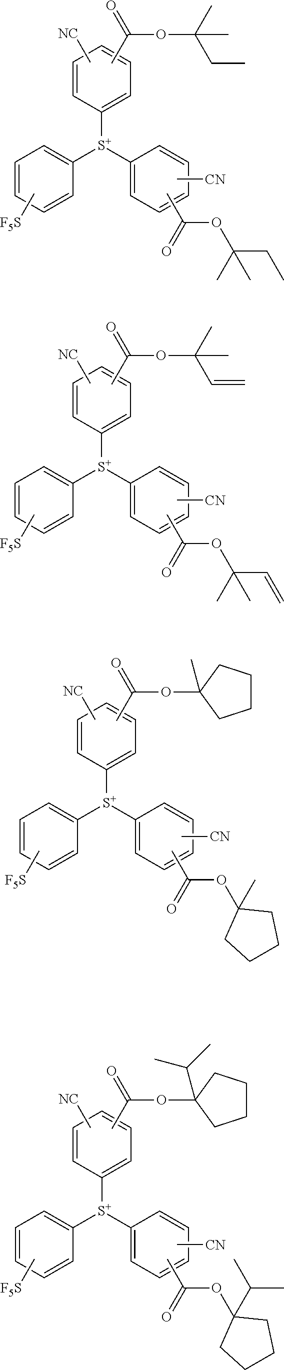

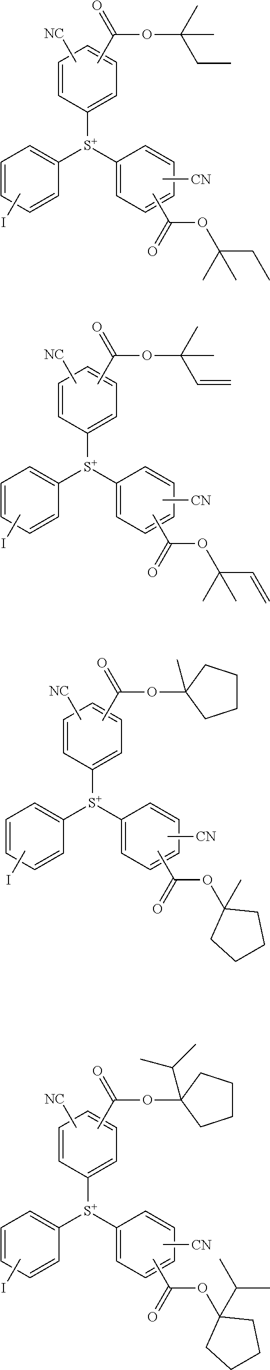

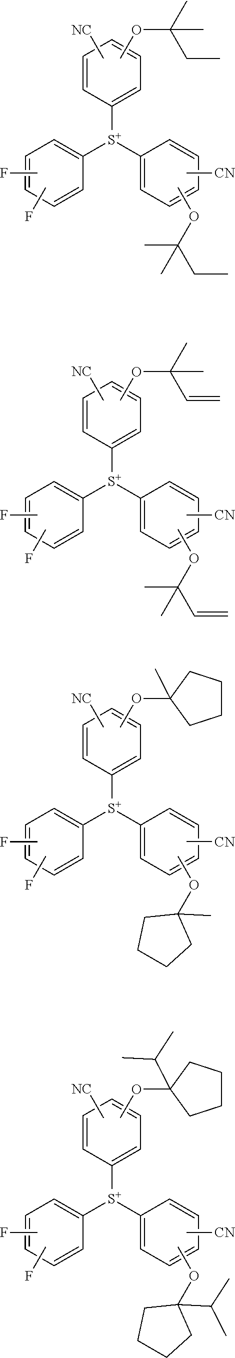

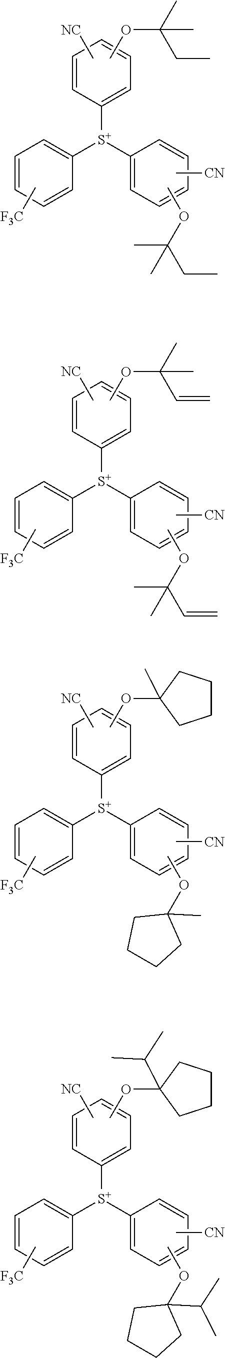

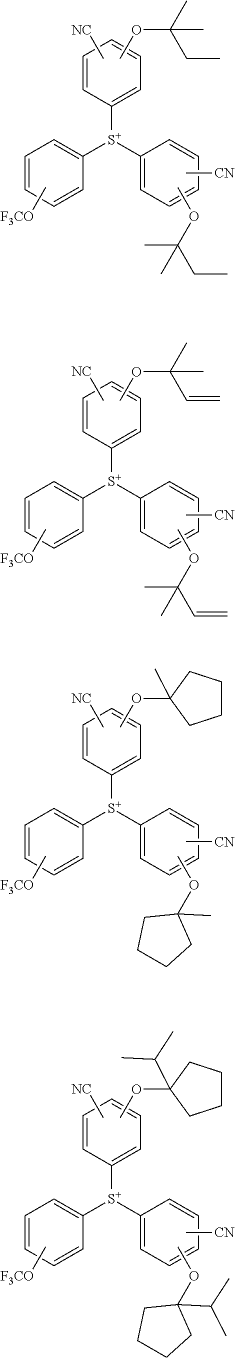

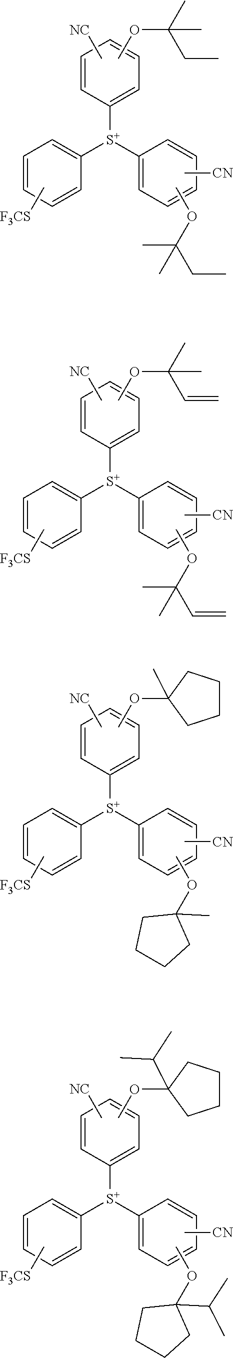

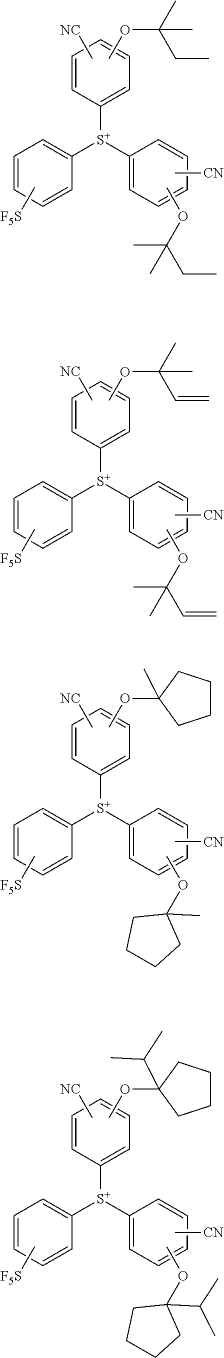

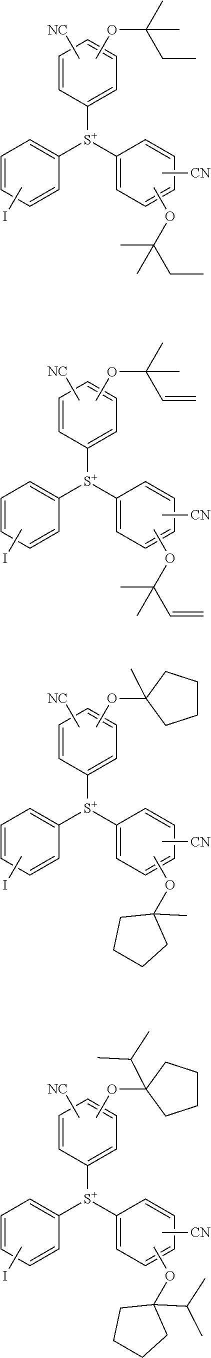

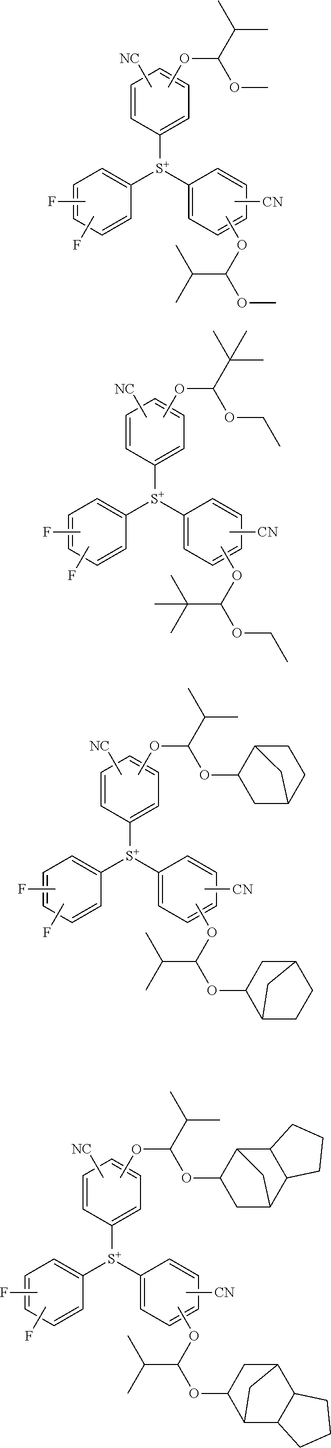

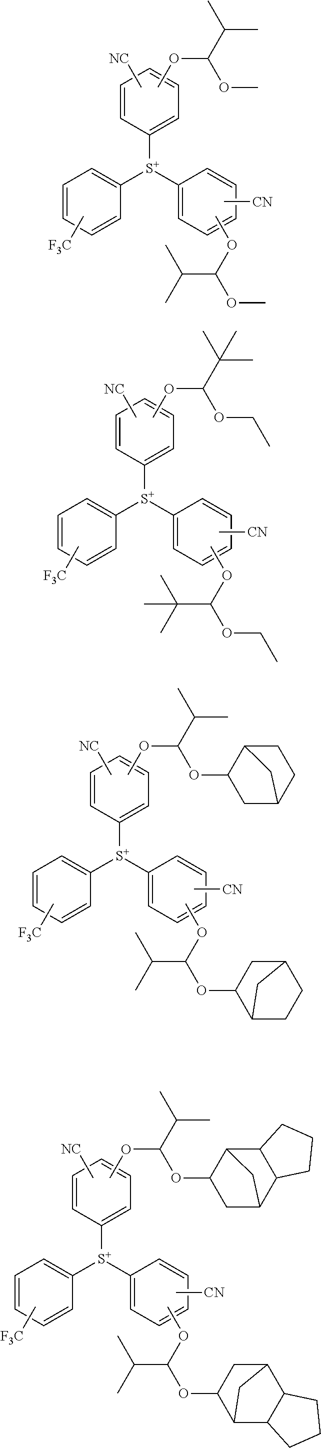

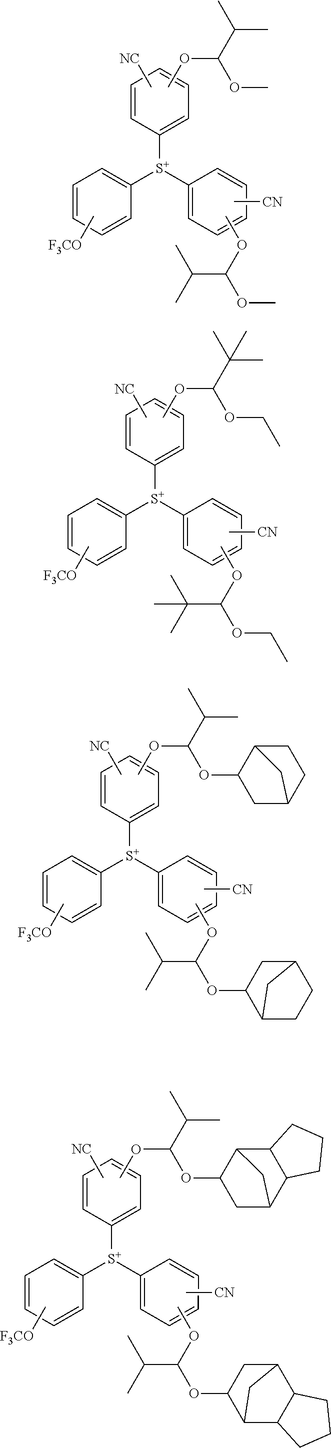

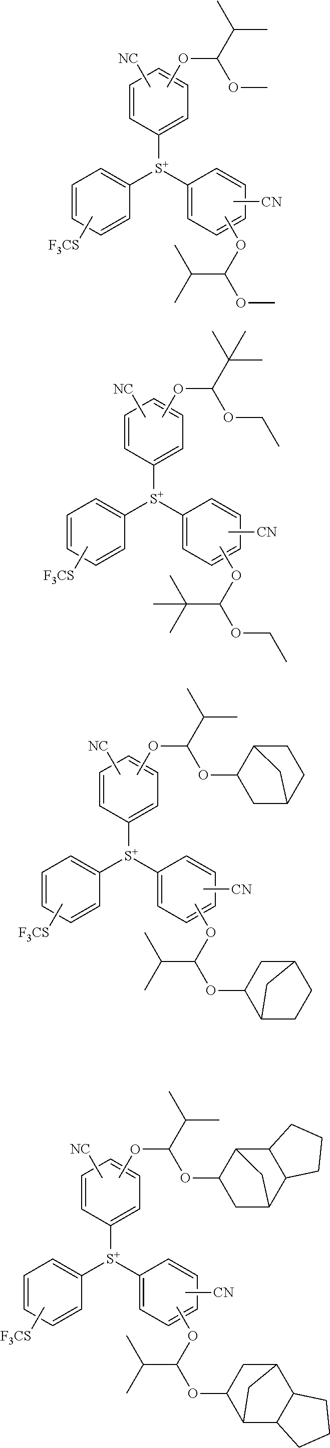

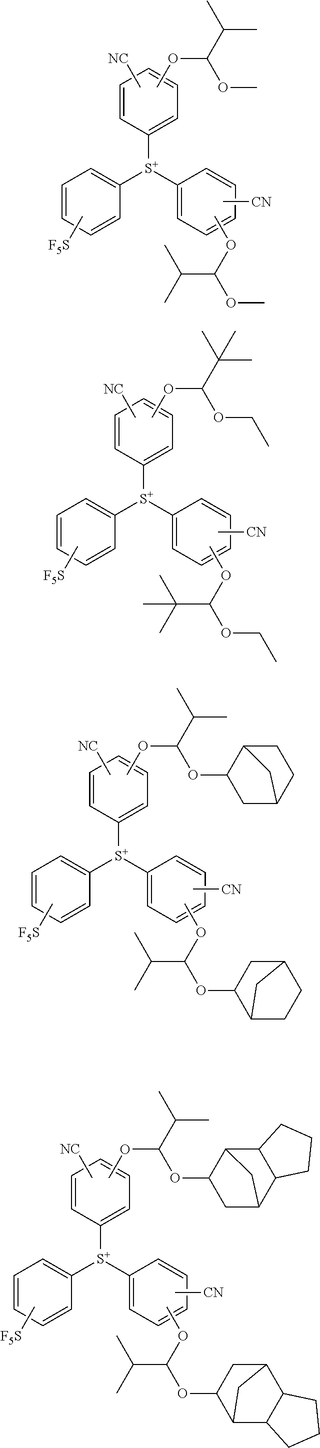

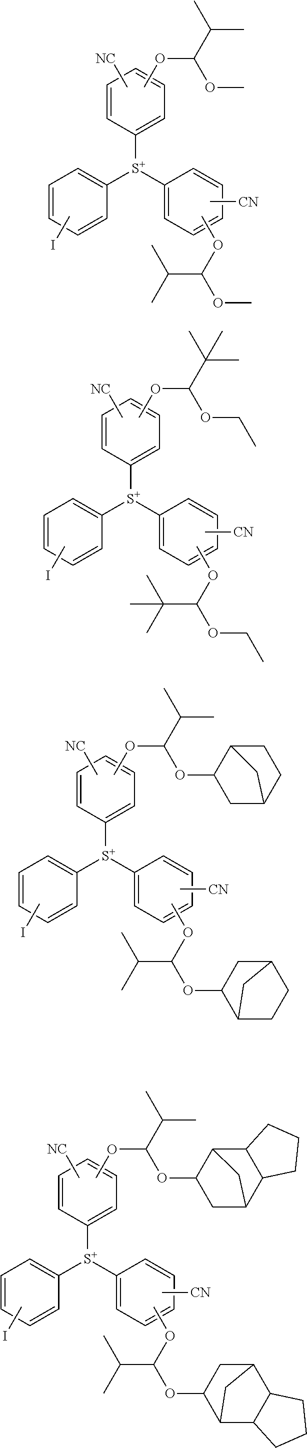

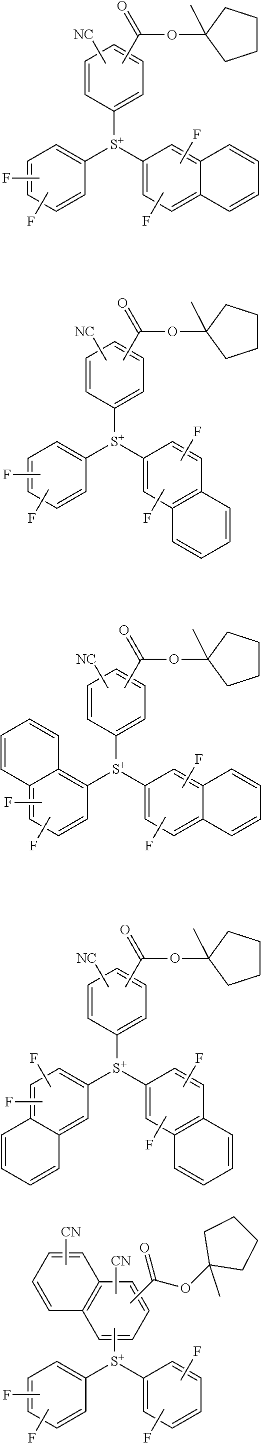

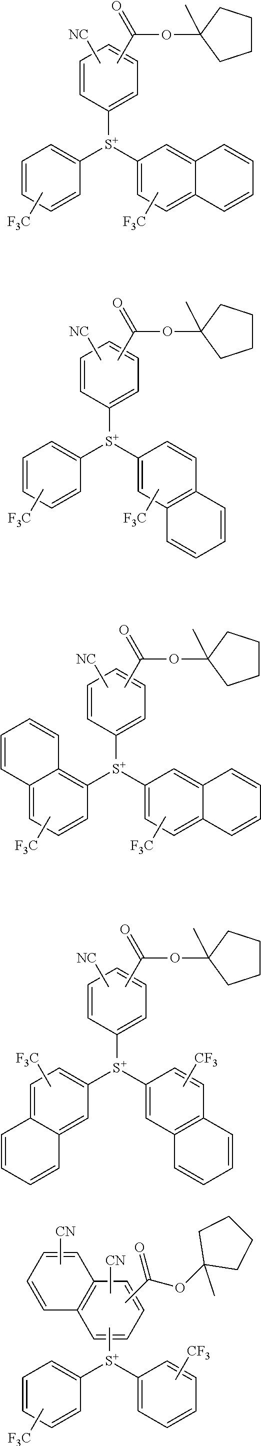

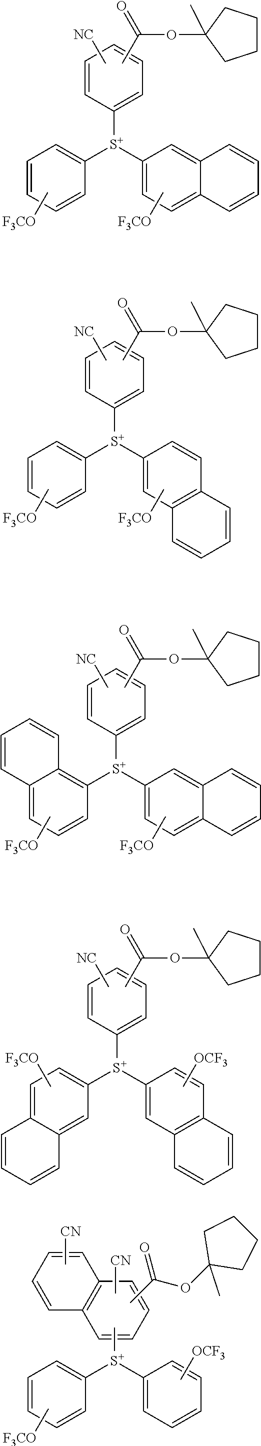

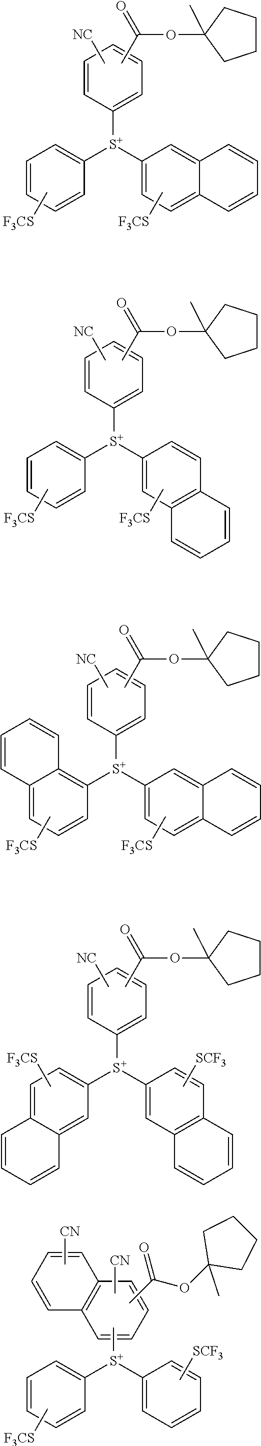

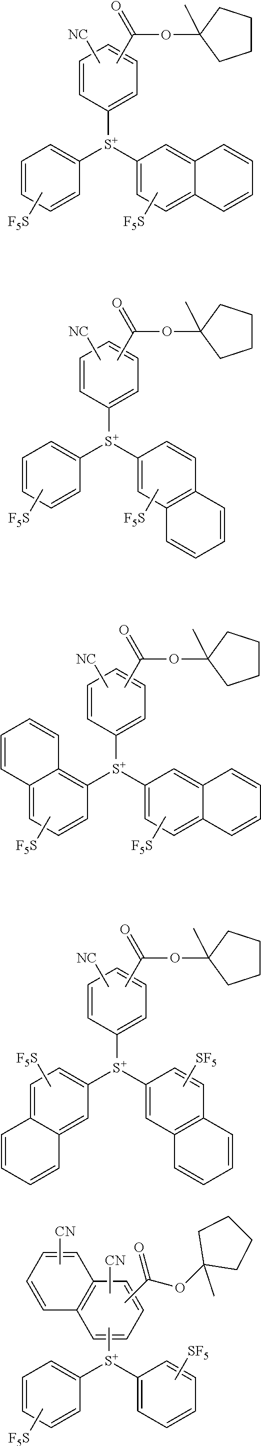

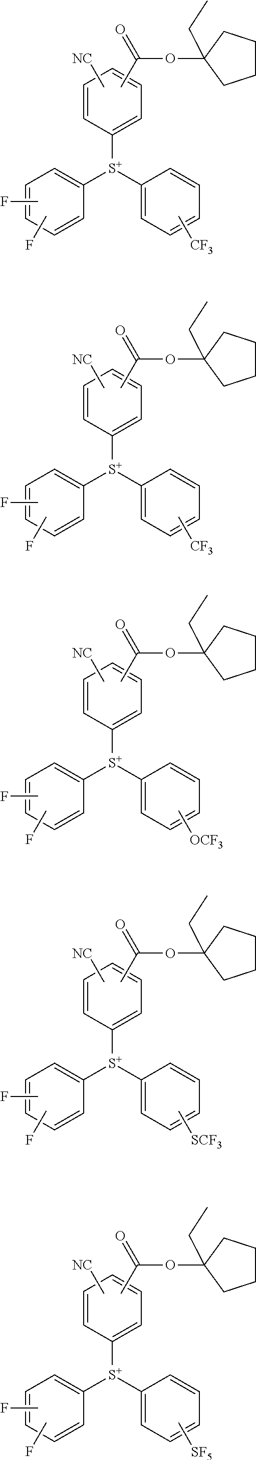

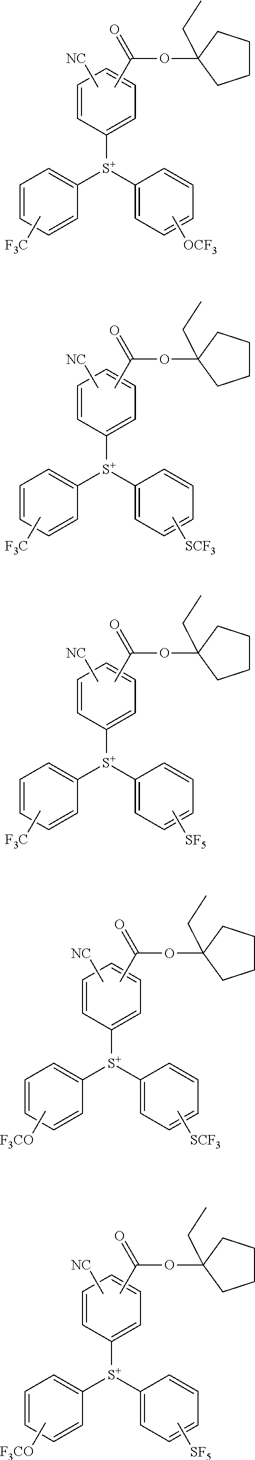

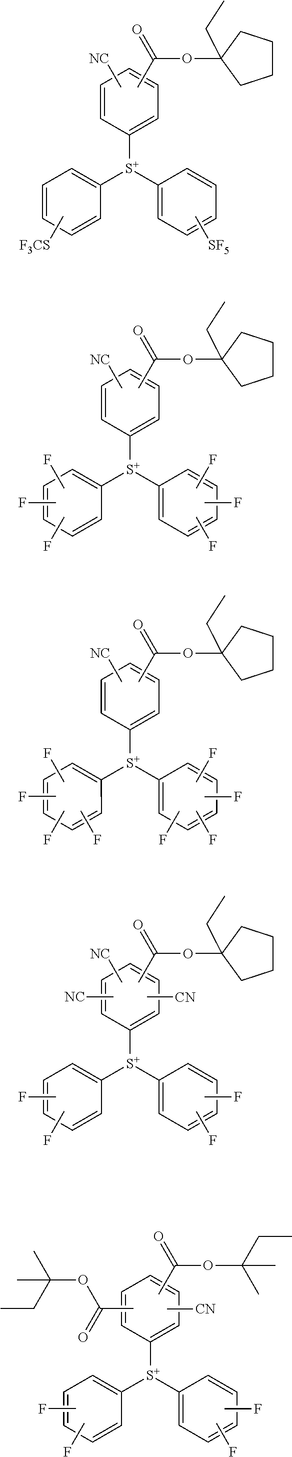

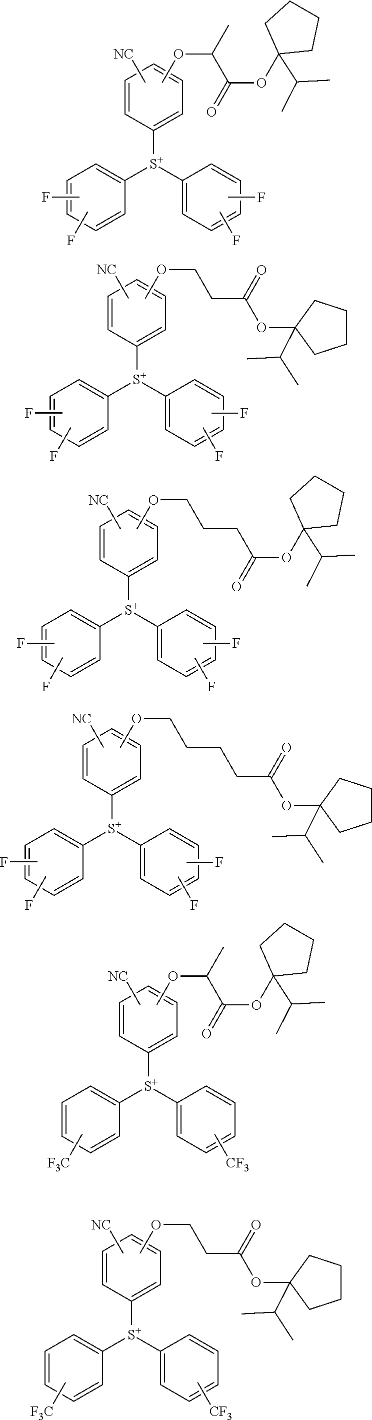

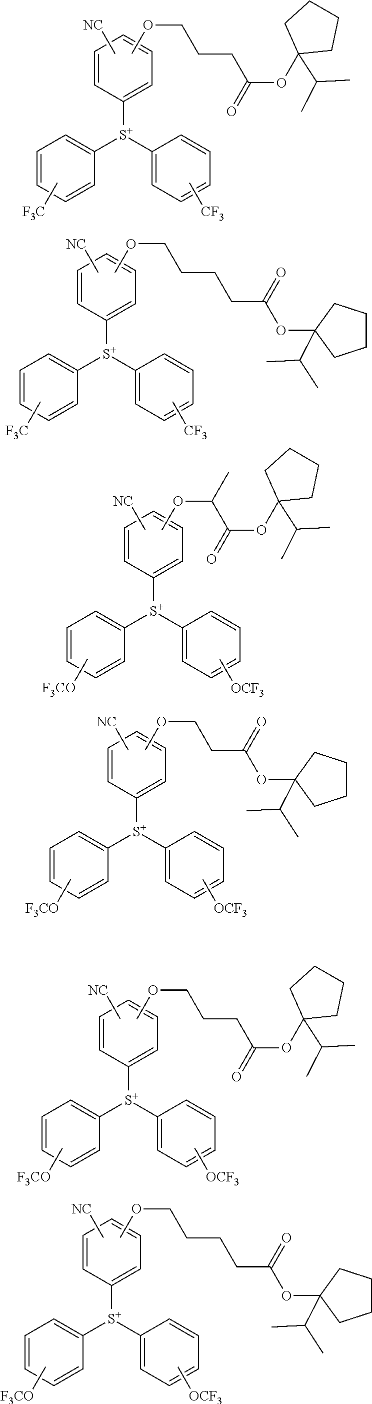

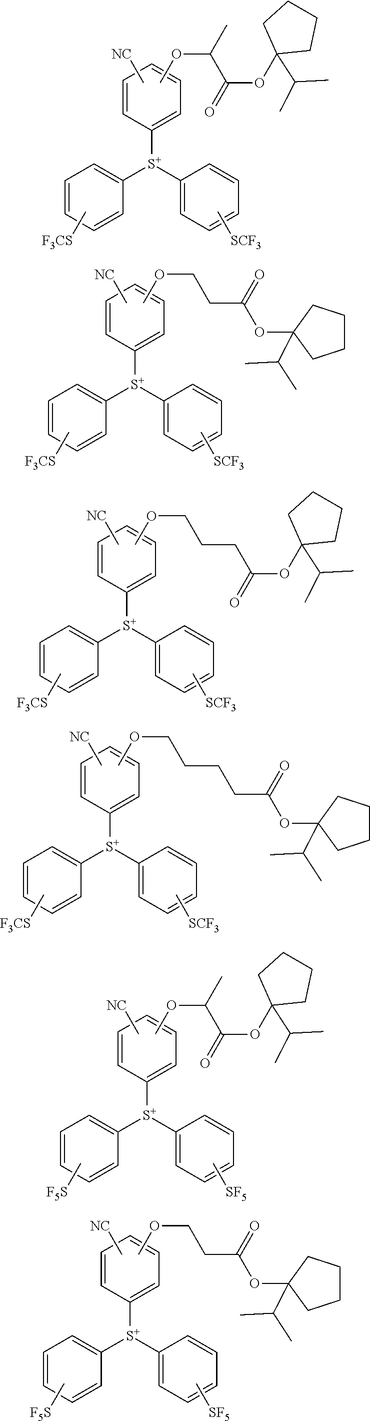

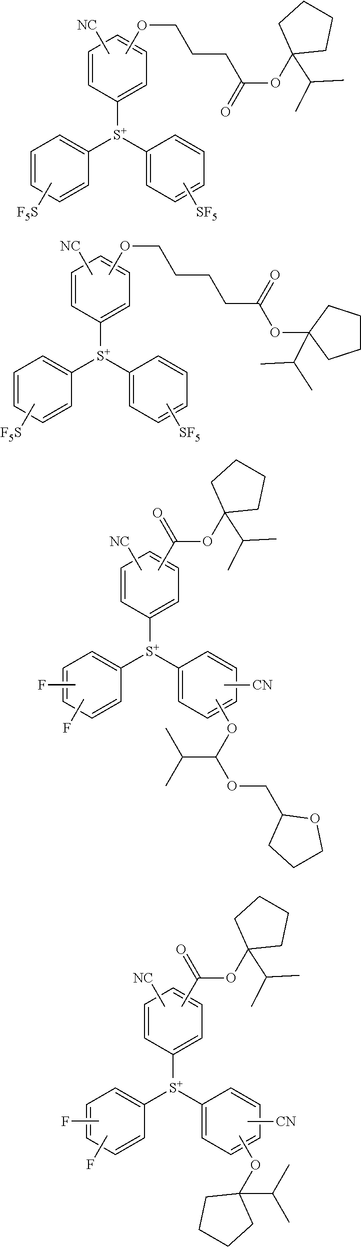

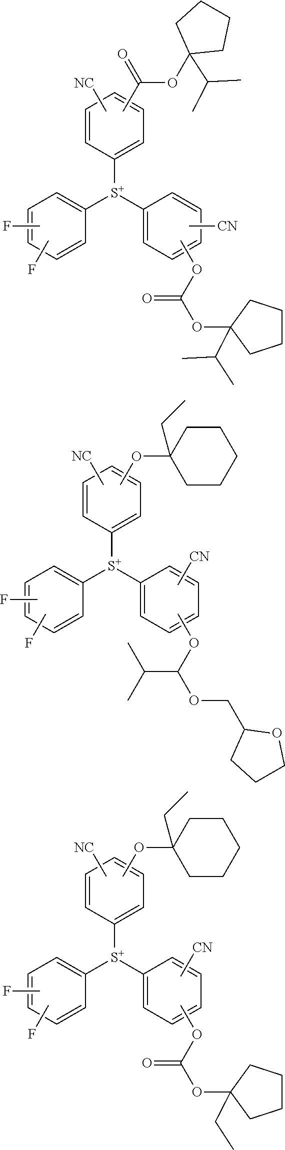

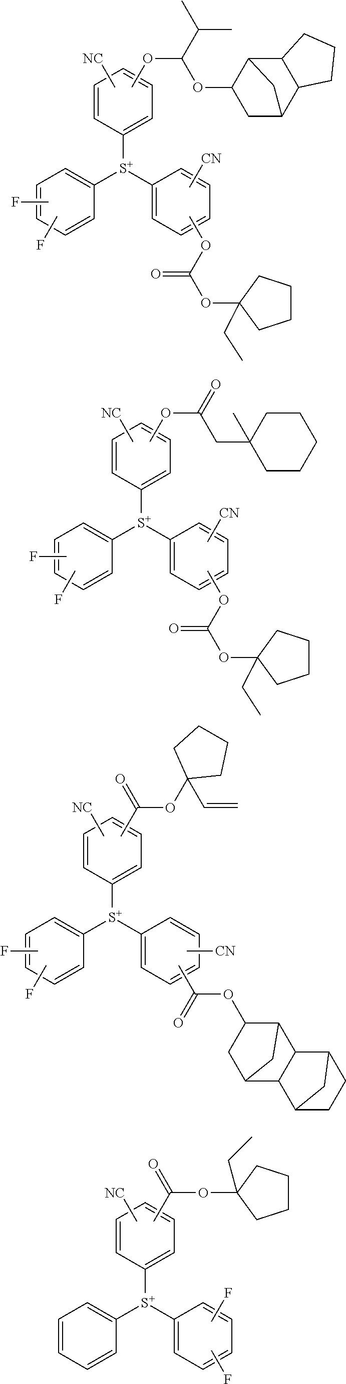

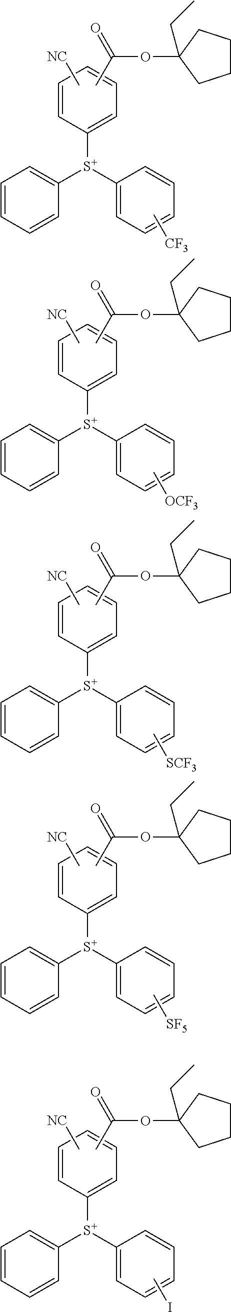

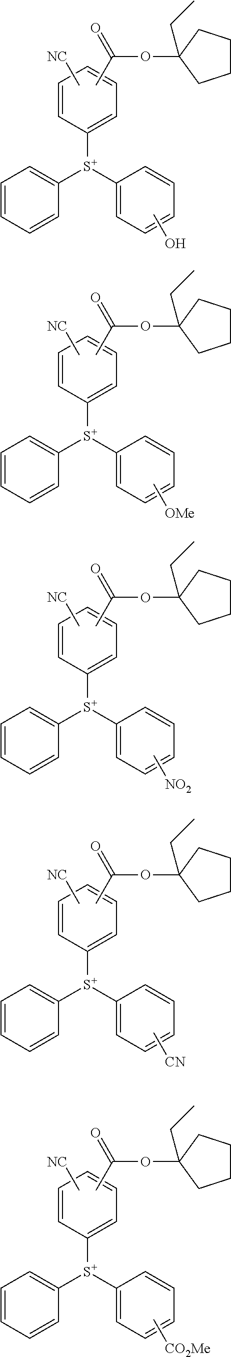

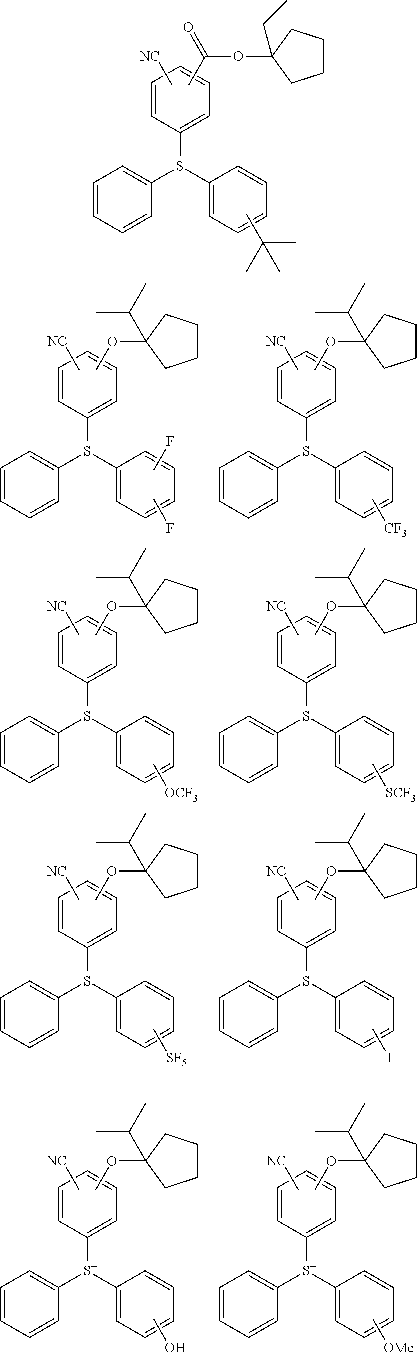

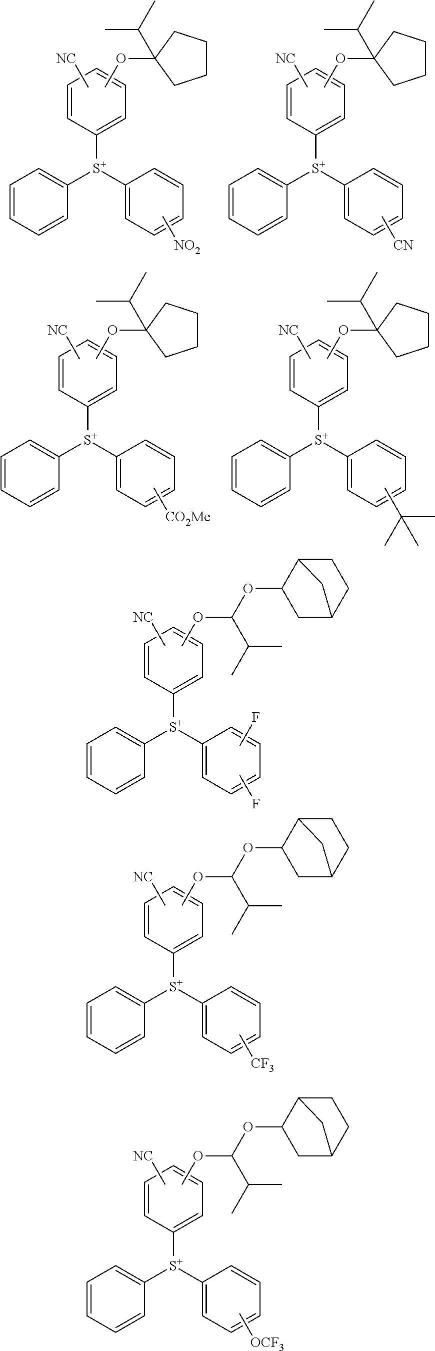

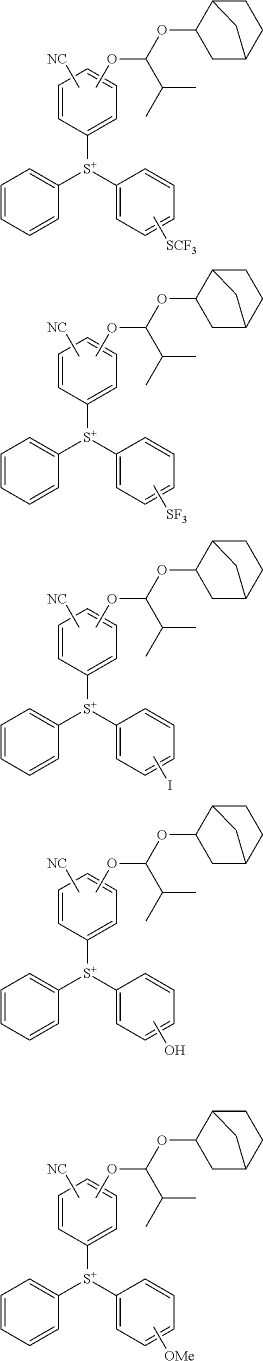

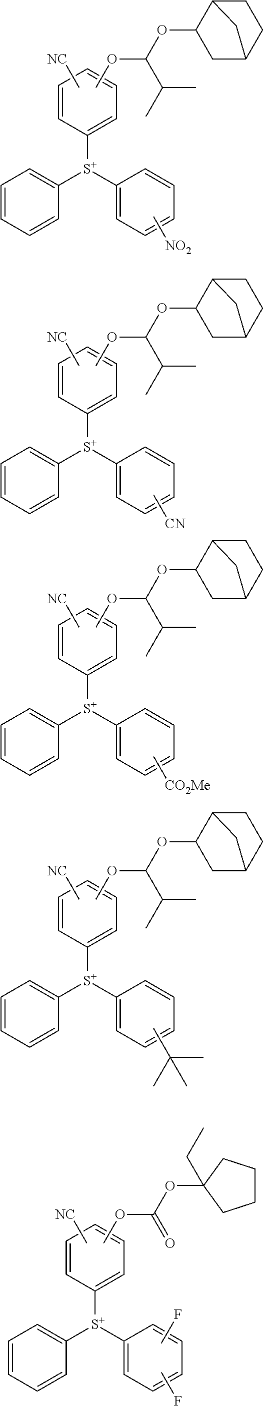

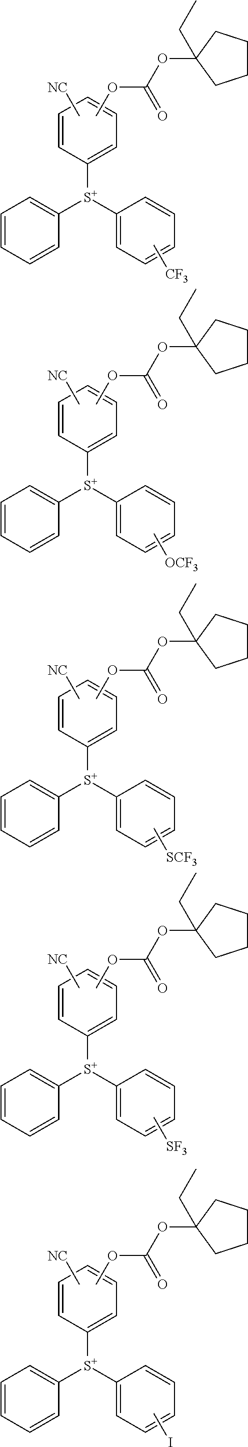

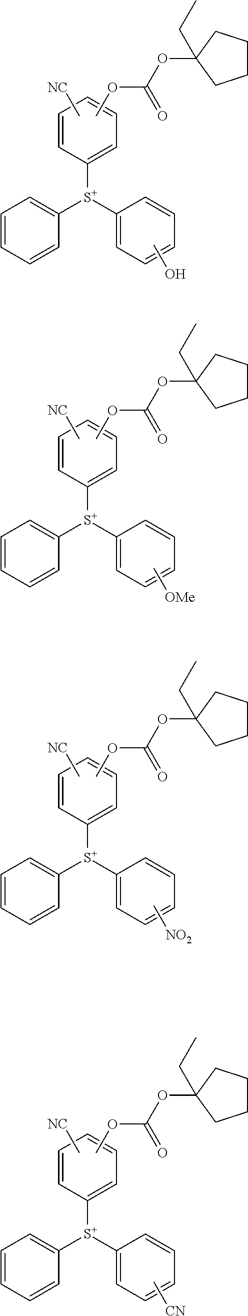

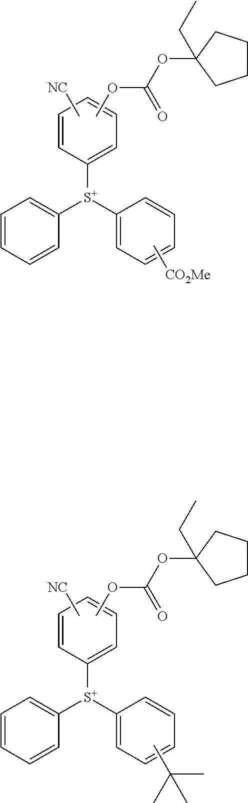

In formulae (A) and (A1), Z⁻ is a fluoroalkanesulfonate anion having a polymerizable group. The fluoroalkanesulfonate anion preferably has the formula (Z1).

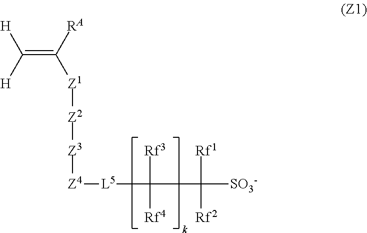

In formula (Z1), k is 0, 1, 2 or 3, and preferably 1.

In formula (Z1), R^{A} is each independently hydrogen, fluorine, methyl or trifluoromethyl. Z¹ is each independently a single bond, optionally substituted phenylene group, naphthylene group or *—C(═O)—O—Z¹¹—. Z¹¹ is a C₁-C₁₀ aliphatic hydrocarbylene group which may contain halogen, hydroxy, ether bond, ester bond or lactone ring, a phenylene group or naphthylene group. Z² is a single bond, ether bond, ester bond, amide bond, sulfonate ester bond, sulfonamide bond, carbonate bond or carbamate bond. Z³ is each independently a single bond, ***—Z³¹—C(═O)—O—, ***—C(═O)—N(H)—Z³¹— or ***—O—Z³¹—. Z³¹ is a C₁-C₂₀ hydrocarbylene group which may contain a heteroatom. Z⁴ is each independently a single bond, ****—Z⁴¹—C(═O)—O—, ****—C(═O)—N(H)—Z⁴¹— or ****—O—Z⁴¹. Z⁴¹ is a C₁-C₂₀ hydrocarbylene group which may contain a heteroatom. * designates a point of attachment to the carbon atom in the backbone. *** designates a point of attachment to Z². **** designates a point of attachment to Z³.

The aliphatic hydrocarbylene group Z¹¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl; and combinations thereof.

The hydrocarbylene groups which may contain a heteroatom, represented by Z³¹ and Z⁴¹, may be saturated or unsaturated, and may be straight, branched or cyclic. Examples thereof are shown below, but are not limited thereto.

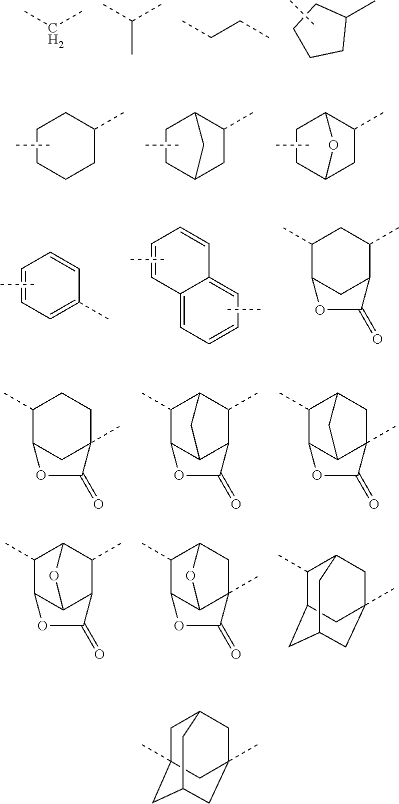

Herein, the broken line denotes a point of attachment.

In formula (Z1), L⁵ is a single bond, ether bond, ester bond, carbonyl group, sulfonate ester bond, sulfonamide bond, carbonate bond or carbamate bond. From the aspect of synthesis, L⁵ is preferably an ether bond, ester bond or carbonyl group, more preferably ester bond or carbonyl group.

In formula (Z1), Rf¹ and Rf² are each independently a fluorine atom or a C₁-C₆ fluorinated saturated hydrocarbyl group. Preferably both Rf¹ and Rf² are fluorine because the generated acid has a higher acid strength.

In formula (Z1), Rf³ and Rf⁴ are each independently hydrogen, fluorine or a C₁-C₆ fluorinated saturated hydrocarbyl group. It is preferred for solvent solubility that at least one of Rf³ and Rf^{V} be trifluoromethyl.

As the fluoroalkanesulfonate anion Z⁻, an anion having the formula (Z2) is also preferred.

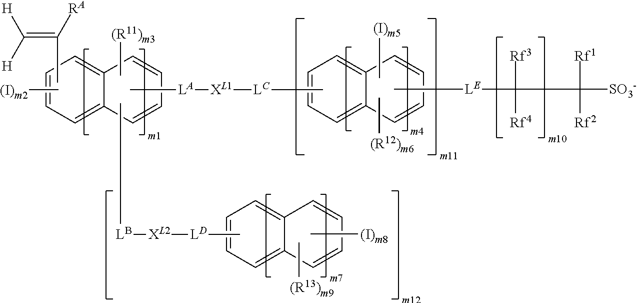

In formula (Z2), m1 is 0 or 1. The relevant structure is a benzene ring in case of m1=0 and a naphthalene ring in case of m1=1. From the aspect of solvent solubility, the benzene ring corresponding to m1=0 is preferred. m2 is 0, 1, 2, 3 or 4. From the aspect of reactant availability, m2 is preferably 0, 1, 2 or 3, more preferably 0, 1 or 2, most preferably 0 or 1. m3 is 0, 1, 2 or 3.

In formula (Z2), m4 is 0 or 1. The relevant structure is a benzene ring in case of m4=0 and a naphthalene ring in case of m4=1. From the aspect of solvent solubility, the benzene ring corresponding to m4=0 is preferred. m5 is 0, 1, 2, 3 or 4. From the aspect of reactant availability, m5 is preferably 0, 1, 2 or 3, more preferably 0, 1 or 2. m6 is 0, 1, 2 or 3.

In formula (Z2), m7 is 0 or 1. The relevant structure is a benzene ring in case of m7=0 and a naphthalene ring in case of m7=1. From the aspect of solvent solubility, the benzene ring corresponding to m7=0 is preferred. m8 is 1, 2, 3 or 4. From the aspect of reactant availability, m8 is preferably 1, 2 or 3, more preferably 1 or 2. m9 is 0, 1, 2 or 3.

In formula (Z2), m10 is 0, 1, 2, 3 or 4, preferably 0, 1, 2 or 3, more preferably 1, 2 or 3, still more preferably 1. m11 is 0 or 1. m12 is 0 or 1.

0≤m2+m3+m12≤4 when m1=0 and 0≤m2+m3+m12≤6 when m1=1. 0≤m5+m6≤4 when m4=0 and 0≤m5+m6≤6 when m4=1. 0≤m8+m9≤5 when m7=0 and 0≤m8+m9≤7 when m7=1. As the number of iodine atoms in the anion is more, the absorption of EUV becomes higher, but solvent solubility becomes poorer. There remains concern that the compound precipitates in the resist composition. For this reason, 1≤m2+m5+m8≤4 is preferred.

In formula (Z2), R^{A} is hydrogen, fluorine, methyl or trifluoromethyl. R^{A} is preferably hydrogen or methyl, more preferably hydrogen.

In formula (Z2), R¹¹ is halogen exclusive of iodine, nitro, cyano, hydroxy, carboxy, a C₁-C₂₀ hydrocarbyl group which may contain a heteroatom, C₁-C₂₀ hydrocarbyloxy group which may contain a heteroatom, C₁-C₂₀ hydrocarbylthio group which may contain a heteroatom or C₂-C₂₀ hydrocarbyloxycarbonyl group which may contain a heteroatom. Suitable halogen atoms exclusive of iodine include fluorine, chlorine, and bromine, with fluorine being more preferred. The hydrocarbyl group or hydrocarbyl moiety in the hydrocarbyloxy, hydrocarbylthio and hydrocarbyloxycarbonyl groups may be saturated or unsaturated, and may be straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety. When m3 is 2 or 3, a plurality of R¹¹ may be identical or different.

When m3 is 2 or 3, two R¹¹ may bond together to form a ring with the carbon atoms to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur, or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

In formula (Z2), R¹² is halogen exclusive of iodine, nitro, cyano, hydroxy, carboxy, a C₁-C₂₀ hydrocarbyl group which may contain a heteroatom, C₁-C₂₀ hydrocarbyloxy group which may contain a heteroatom, C₁-C₂₀ hydrocarbylthio group which may contain a heteroatom or C₂-C₂₀ hydrocarbyloxycarbonyl group which may contain a heteroatom. Suitable halogen atoms exclusive of iodine include fluorine, chlorine, and bromine. The hydrocarbyl group or hydrocarbyl moiety in the hydrocarbyloxy, hydrocarbylthio and hydrocarbyloxycarbonyl groups may be saturated or unsaturated, and may be straight, branched or cyclic. Examples thereof are as exemplified above for the hydrocarbyl group R¹¹, but are not limited thereto. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety. When m6 is 2 or 3, a plurality of R¹² may be identical or different.

When m6 is 2 or 3, two R¹² may bond together to form a ring with the carbon atoms to which they are attached. The ring is preferably 5 to 8-membered.

In formula (Z2), R¹³ is halogen exclusive of iodine, nitro, cyano, hydroxy, carboxy, a C₁-C₂₀ hydrocarbyl group which may contain a heteroatom, C₁-C₂₀ hydrocarbyloxy group which may contain a heteroatom, C₁-C₂₀ hydrocarbylthio group which may contain a heteroatom or C₂-C₂₀ hydrocarbyloxycarbonyl group which may contain a heteroatom. Suitable halogen atoms exclusive of iodine include fluorine, chlorine, and bromine. The hydrocarbyl group or hydrocarbyl moiety in the hydrocarbyloxy, hydrocarbylthio and hydrocarbyloxycarbonyl groups may be saturated or unsaturated, and may be straight, branched or cyclic. Examples thereof are as exemplified above for the hydrocarbyl group R¹¹, but are not limited thereto. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety. When m9 is 2 or 3, a plurality of R¹³ may be identical or different.

When m9 is 2 or 3, two R¹³ may bond together to form a ring with the carbon atoms to which they are attached. The ring is preferably 5 to 8-membered.

In formula (Z2), L^{A}, L^{B}, L^{C}, L^{D} and L^{E} are each independently a single bond, ether bond, ester bond, sulfonate ester bond, amide bond, sulfonamide bond, carbonate bond or carbamate bond. L^{A} is preferably a single bond, ether bond, ester bond or sulfonate ester bond, more preferably an ether bond, ester bond, or sulfonate ester bond. L^{B} is preferably a single bond, ether bond, ester bond, amide bond, sulfonamide bond or sulfonate ester bond, more preferably an ester bond or sulfonate ester bond. L^{C} is preferably a single bond, ether bond, ester bond, amide bond or sulfonate ester bond, more preferably a single bond, ether bond, or ester bond. L^{D} is preferably a single bond, ether bond, ester bond, amide bond or sulfonate ester bond, more preferably a single bond, ether bond, or ester bond. L^{E} is preferably a single bond, ether bond, ester bond or sulfonate ester bond, more preferably a single bond, ether bond, or ester bond.

When m12=1, L^{A} and L^{B} are preferably attached to adjacent carbon atoms on the aromatic ring. Then, a higher sensitivity is expectable because a substituent containing the fluorosulfonate anion structure and a substituent containing an iodized aromatic ring are present at spatially closer positions.

In formula (Z2), X^{L1} and X^{L2} are each independently a single bond or a C₁-C₄₀ hydrocarbylene group which may contain a heteroatom. The hydrocarbylene group may be straight, branched or cyclic. Examples thereof include alkanediyl, cyclic saturated hydrocarbylene, and arylene groups. Suitable heteroatoms include oxygen, nitrogen, and sulfur.

Examples of the C₁-C₄₀ hydrocarbylene group which may contain a heteroatom, represented by X^{L1} and X^{L}2, are shown below, but are not limited thereto. Herein, * designates a point of attachment to L^{A} and L^{C}, or L^{B} and L^{D}.

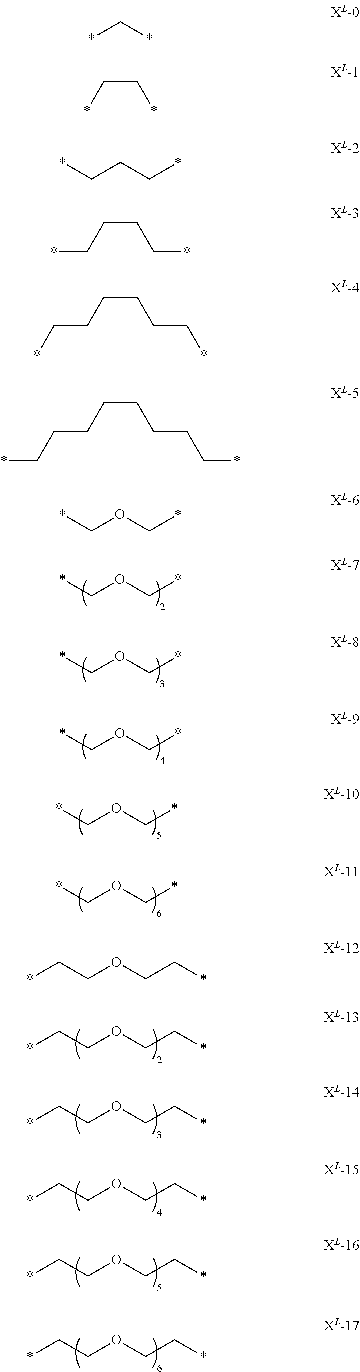

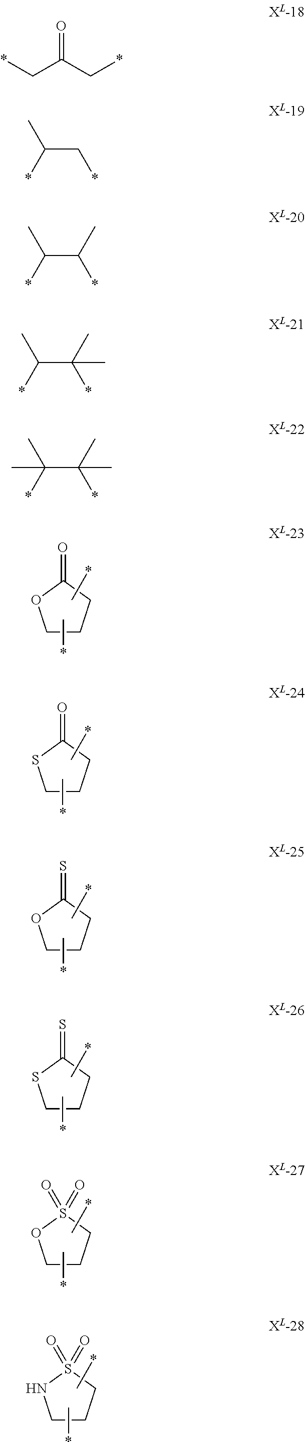

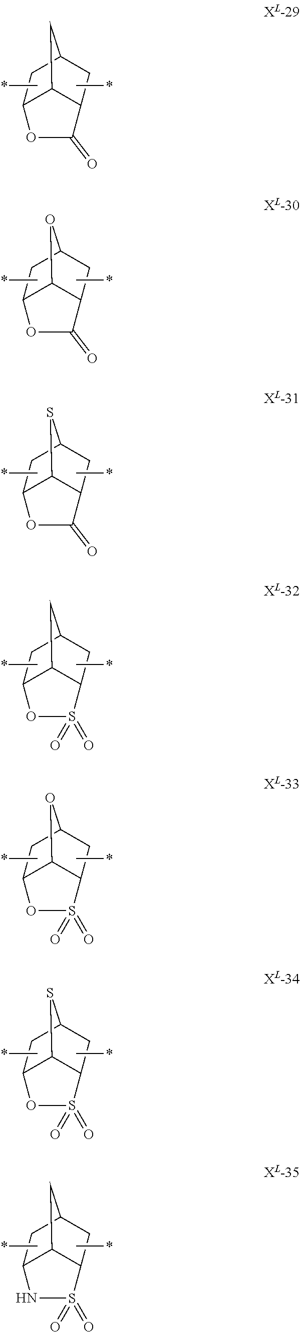

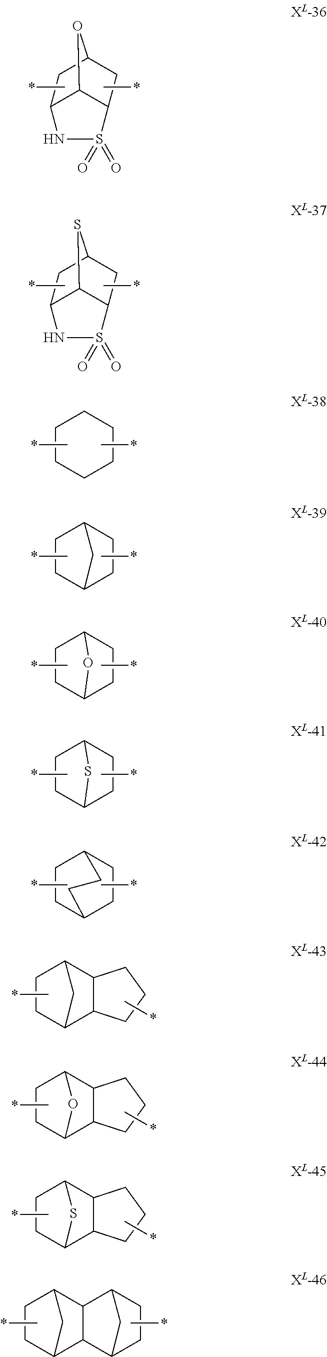

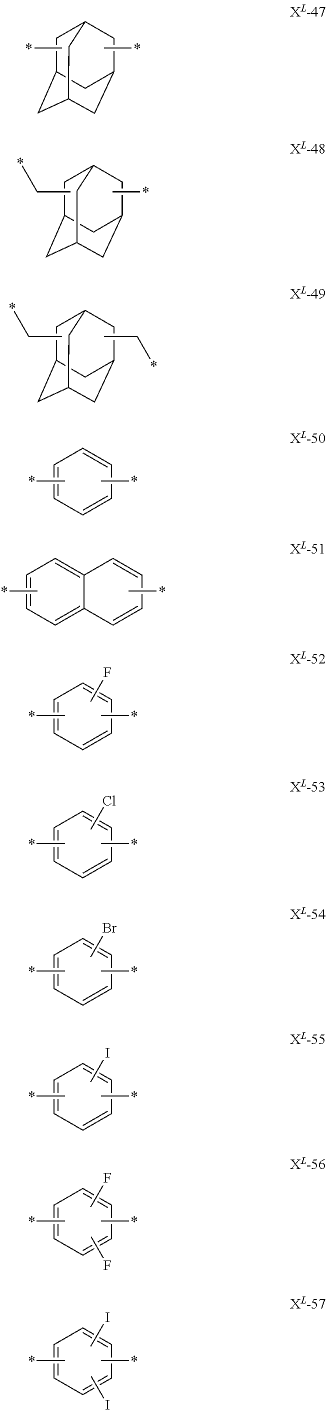

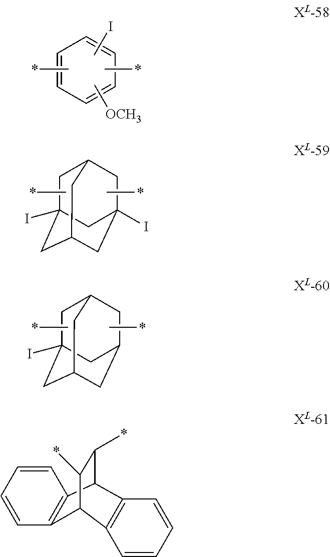

Of these, X^{L}-0 to X^{L}-22, X^{L}-29 to X^{L}-34, and X^{L}-47 to X^{L}-61 are preferred.

Provided that, in formula (Z2), m11 and m12 are not simultaneously 0, and L^{A}, L^{B}, L^{C}, L^{D}, X^{L1} and X^{L2} are not simultaneously single bonds.

In formula (Z2), Rf¹ and Rf² are each independently a fluorine atom or a C₁-C₆ fluorinated saturated hydrocarbyl group. The C₁-C₆ fluorinated saturated hydrocarbyl group is preferably trifluoromethyl. More preferably, both Rf¹ and Rf² are fluorine.

In formula (Z2), Rf³ and Rf⁴ are each independently hydrogen, fluorine or a C₁-C₆ fluorinated saturated hydrocarbyl group. The C₁-C₆ fluorinated saturated hydrocarbyl group is preferably trifluoromethyl.

Preferred examples of the partial structure: —[C(Rf³)(Rf⁴)]ₘ₁₀—C(Rf¹)(Rf²)—SO₃⁻ in formula (Z2) are shown below, but are not limited thereto. Herein, * designates a point of attachment to L^{E}.

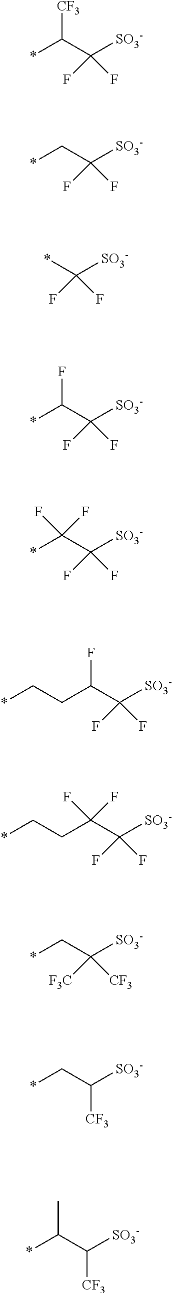

Of these, Acid-1 to Acid-7 are preferred, with Acid-1 to Acid-3, Acid-6, and Acid-7 being more preferred.

Examples of the anion Z⁻ are shown below, but are not limited thereto. Herein, R^{A} and Rf⁴ are as defined above, and Me is methyl. The positions of attachment of substituents on the aromatic ring are interchangeable.

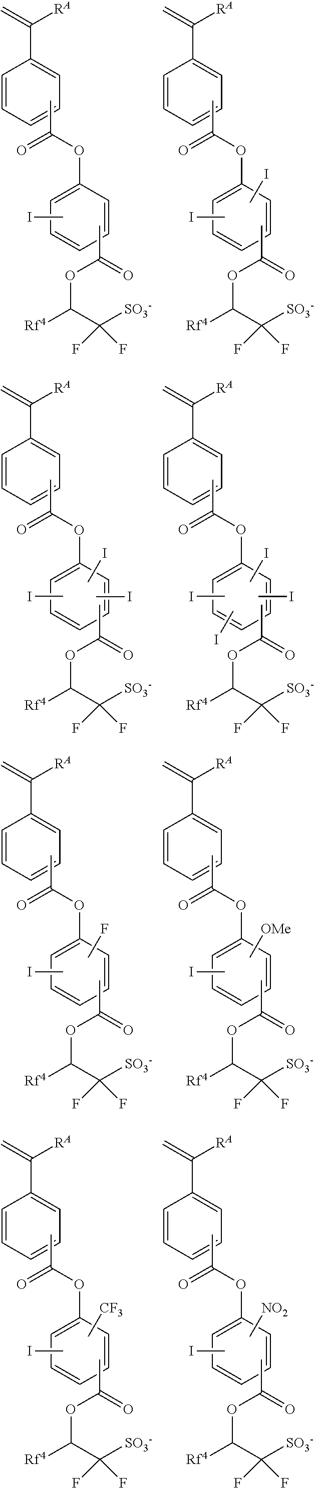

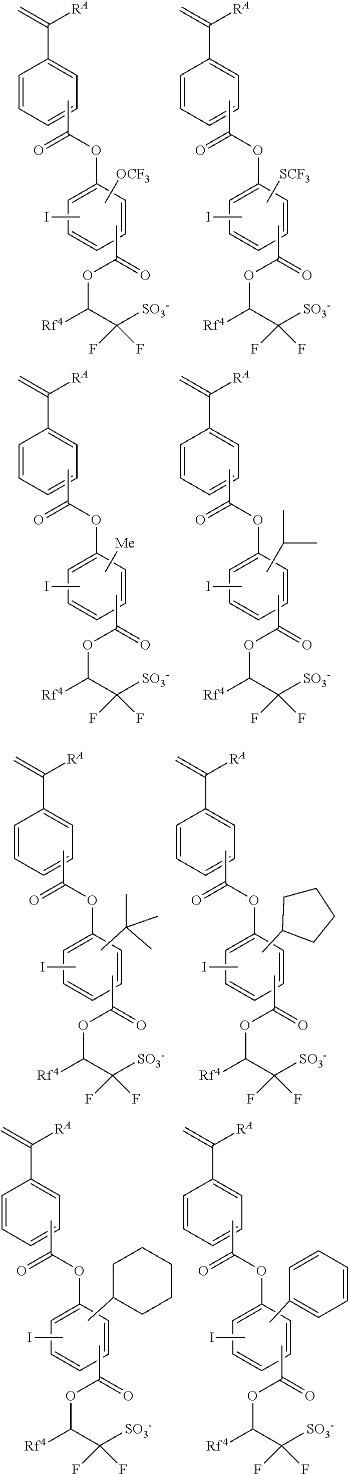

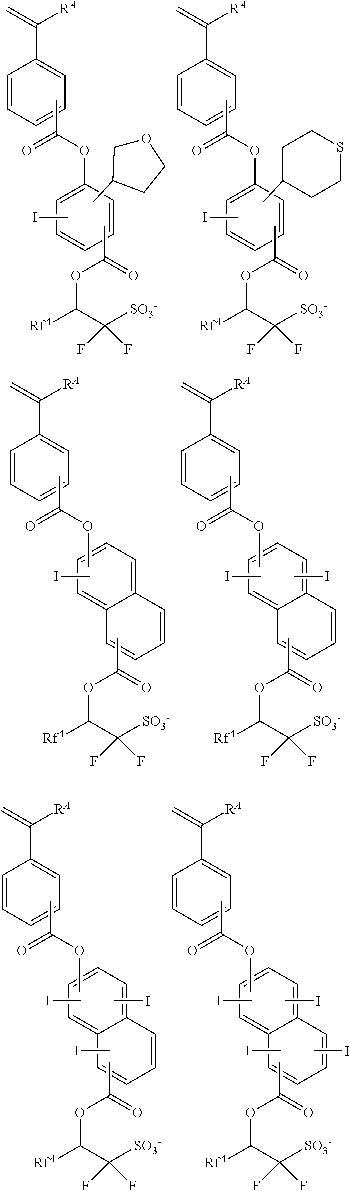

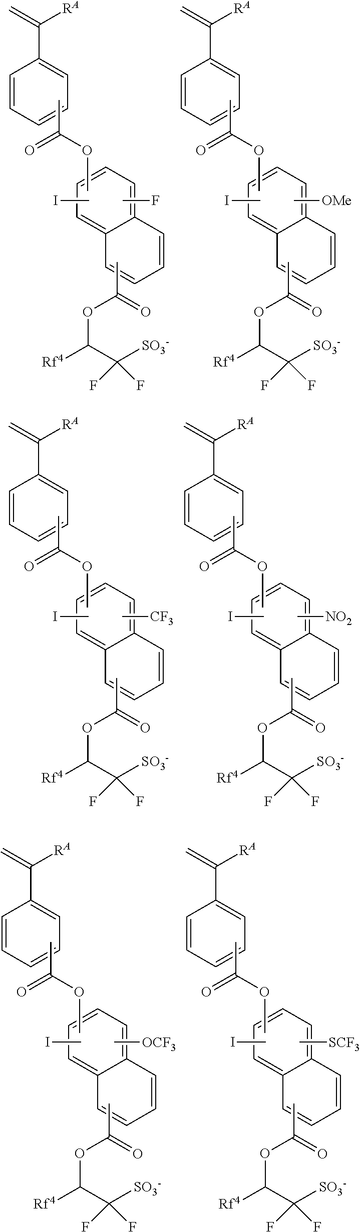

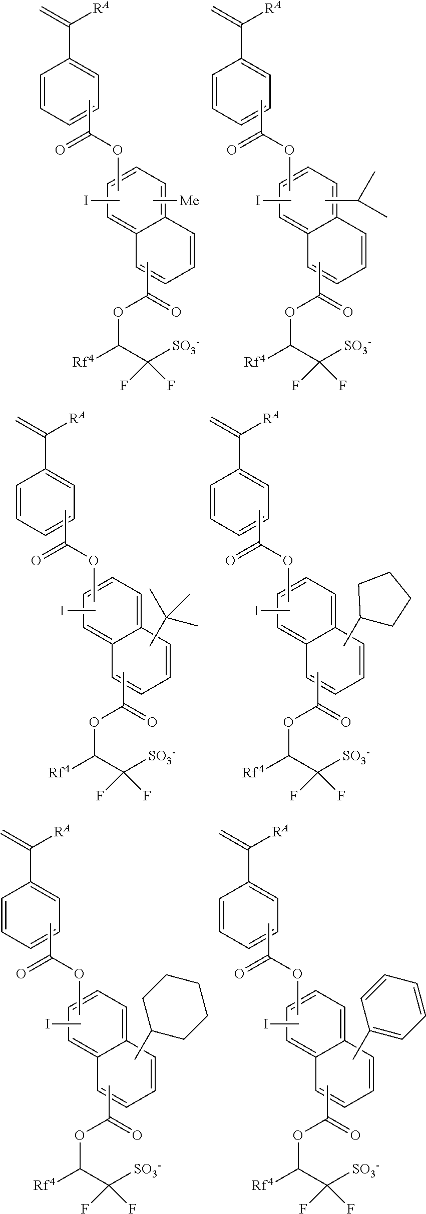

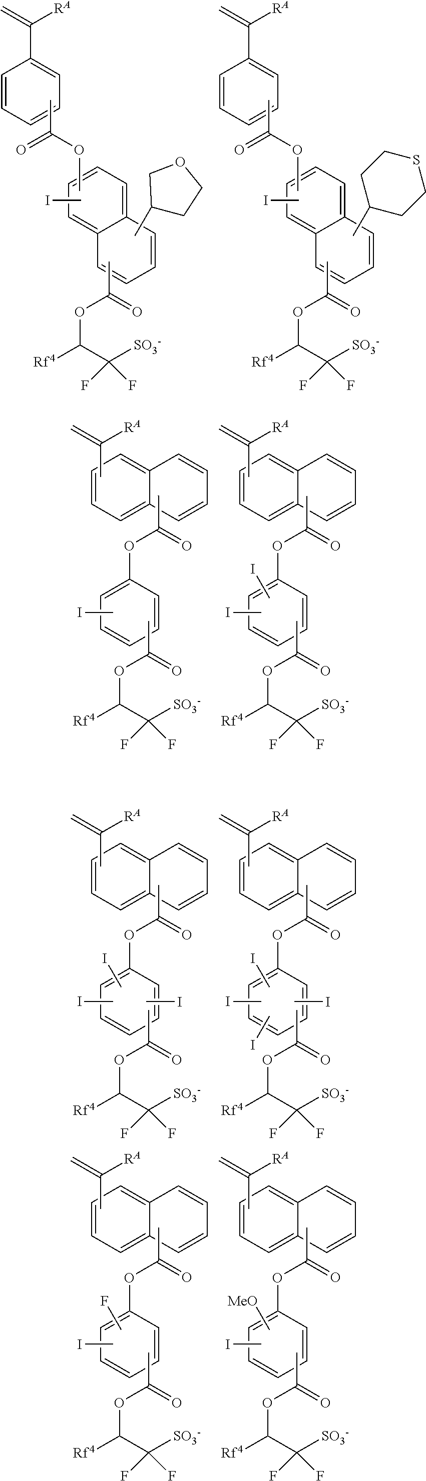

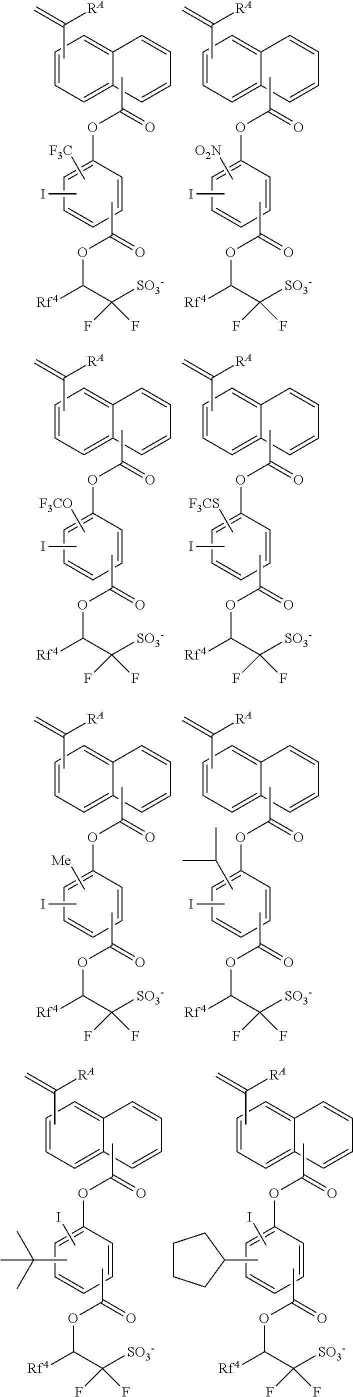

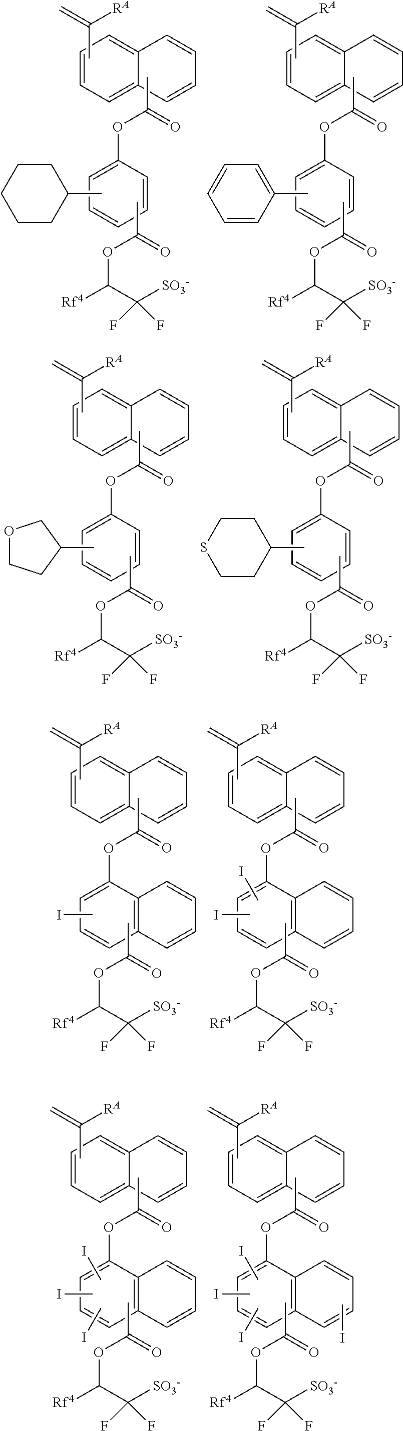

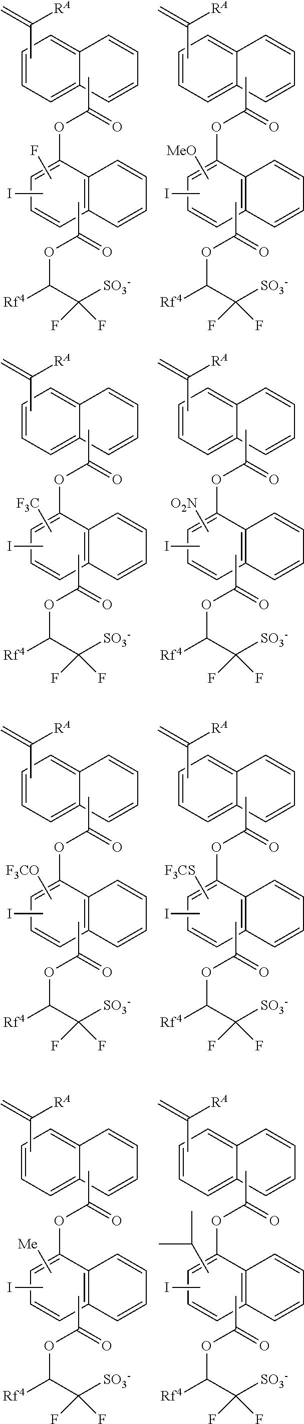

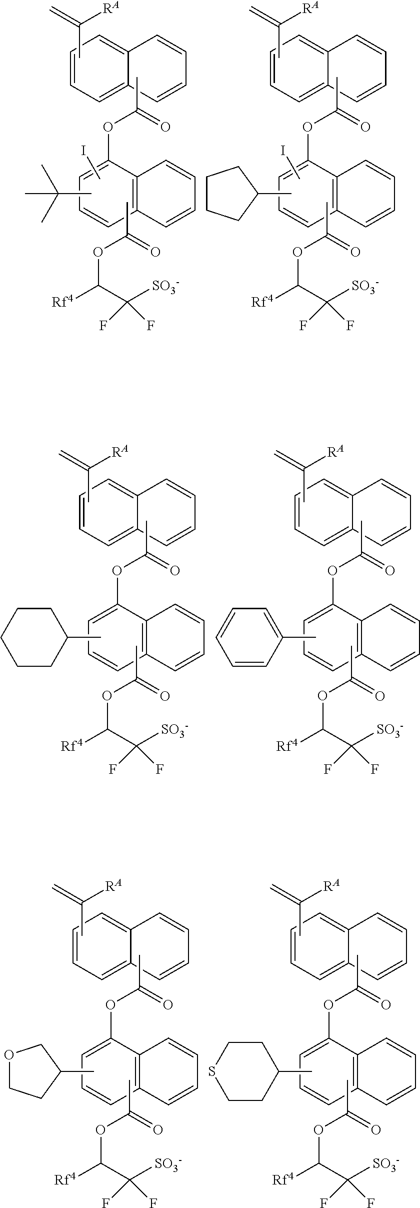

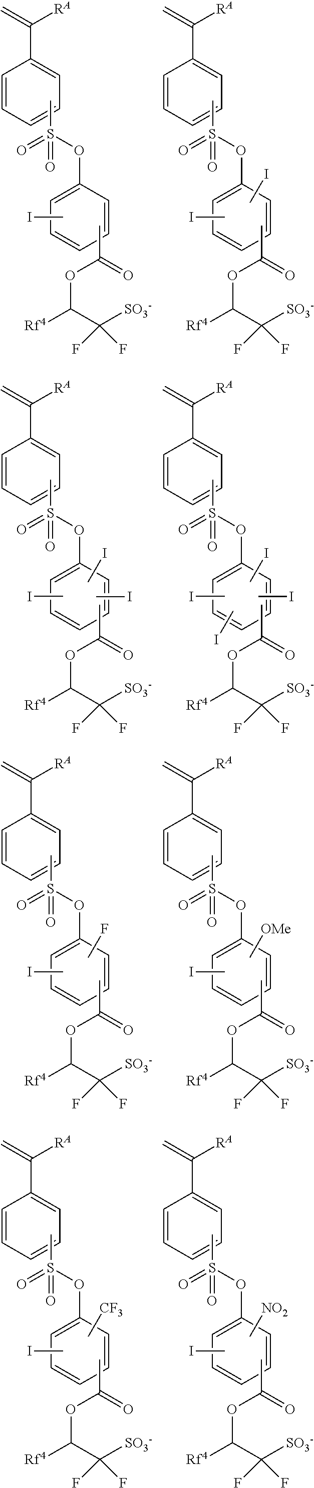

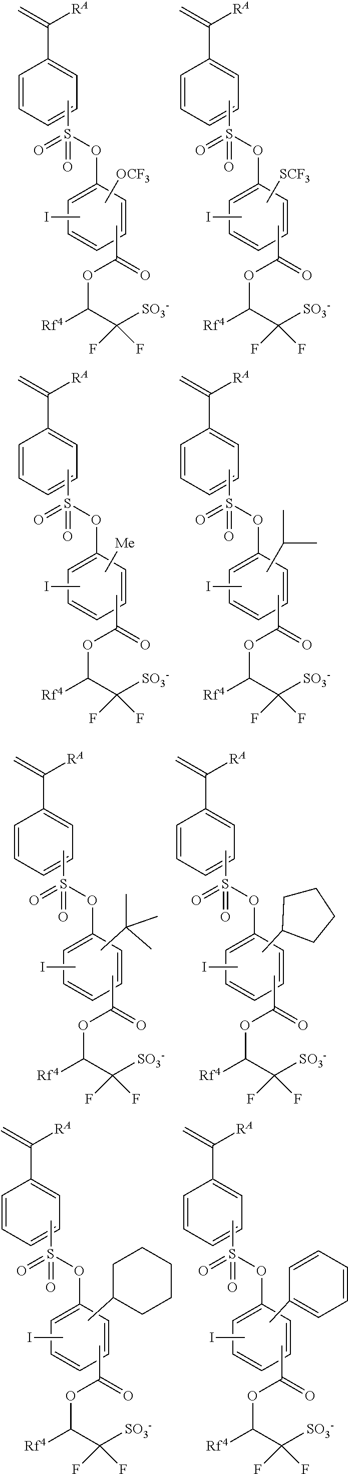

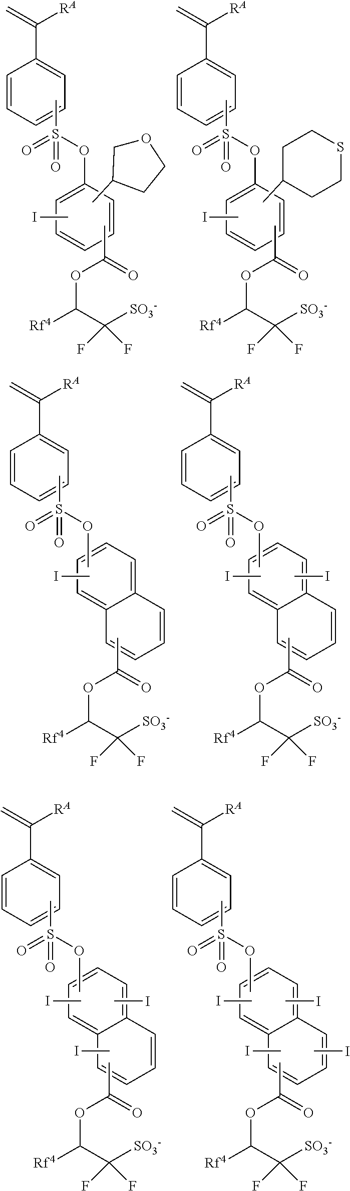

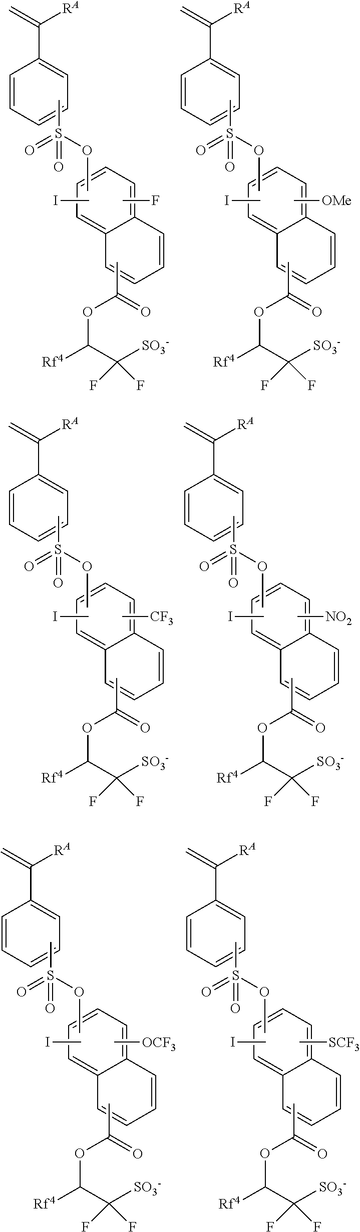

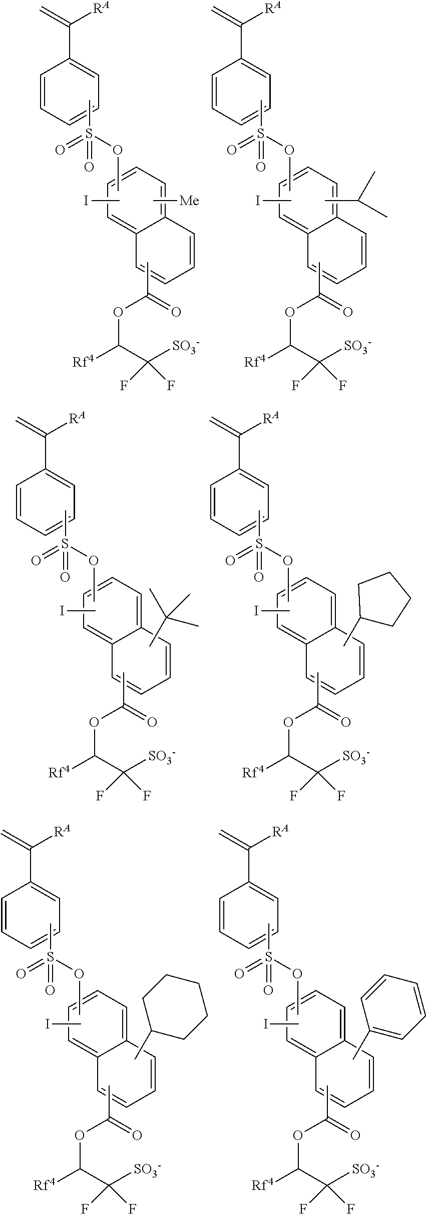

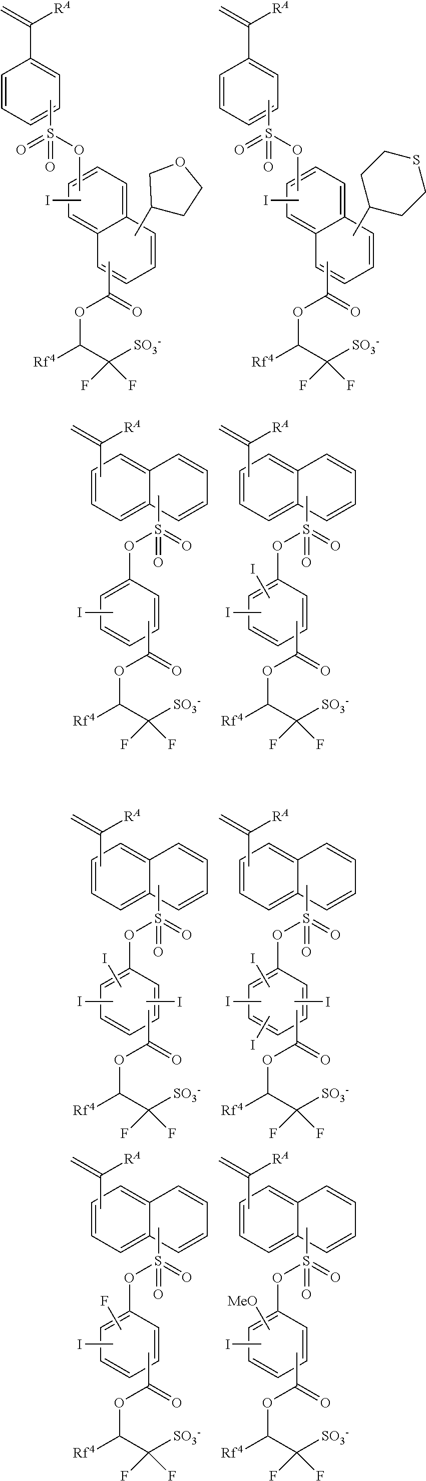

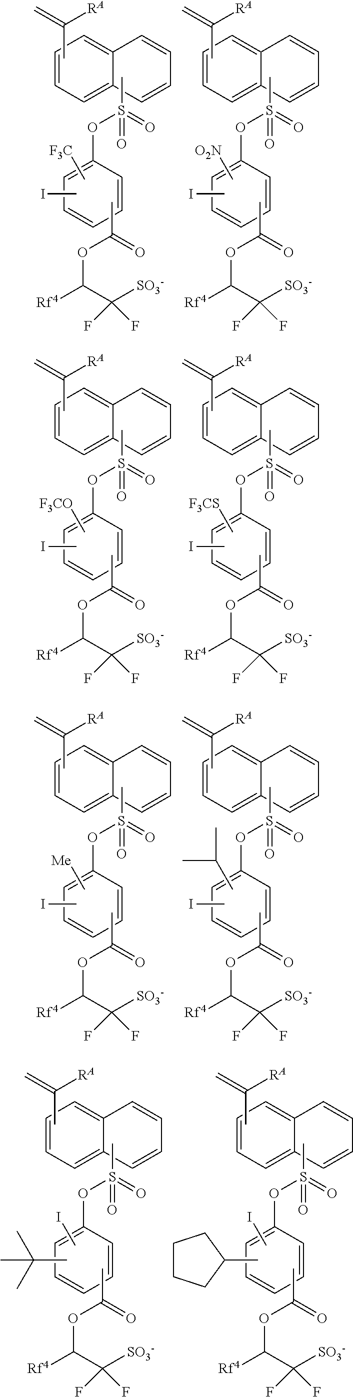

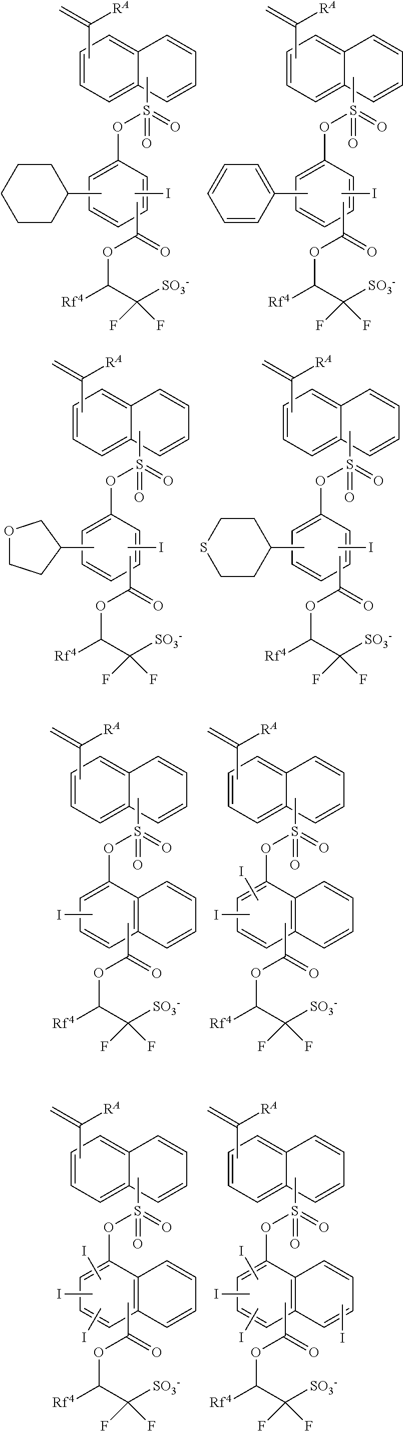

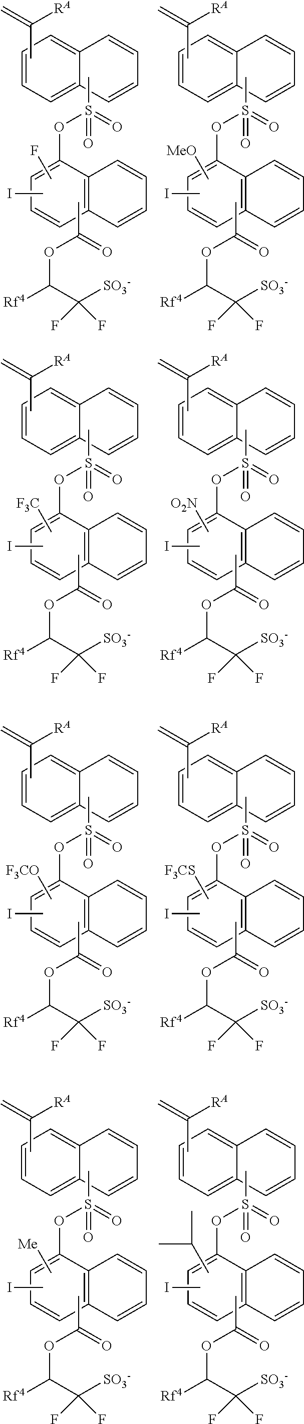

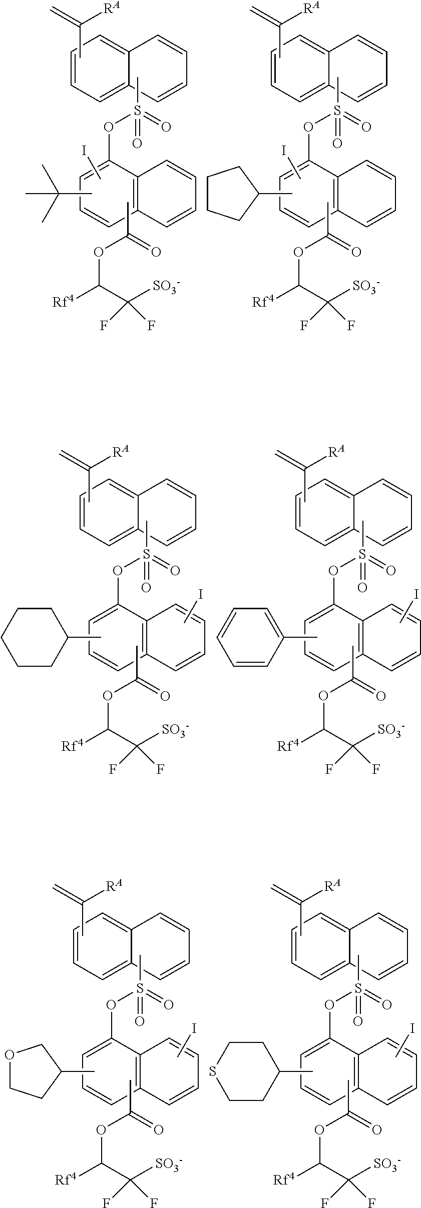

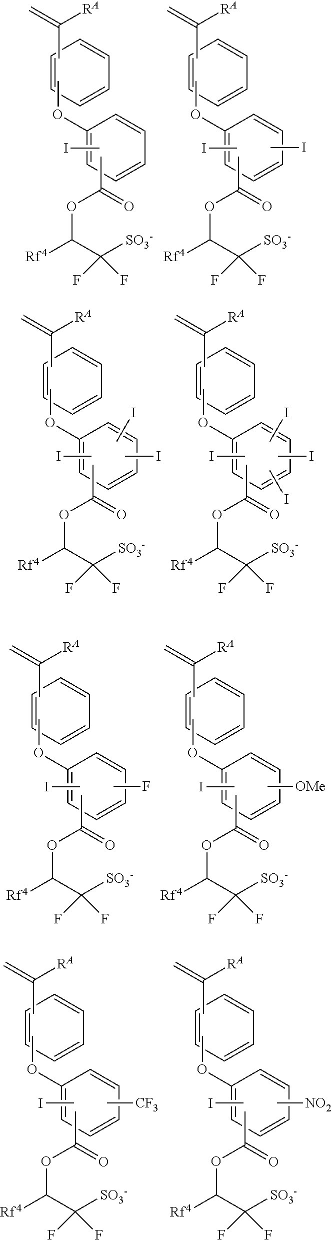

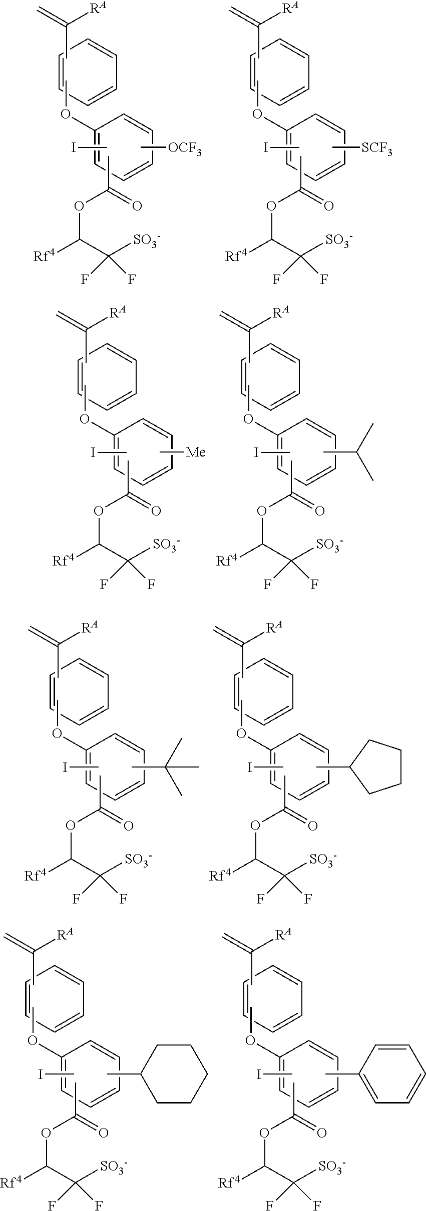

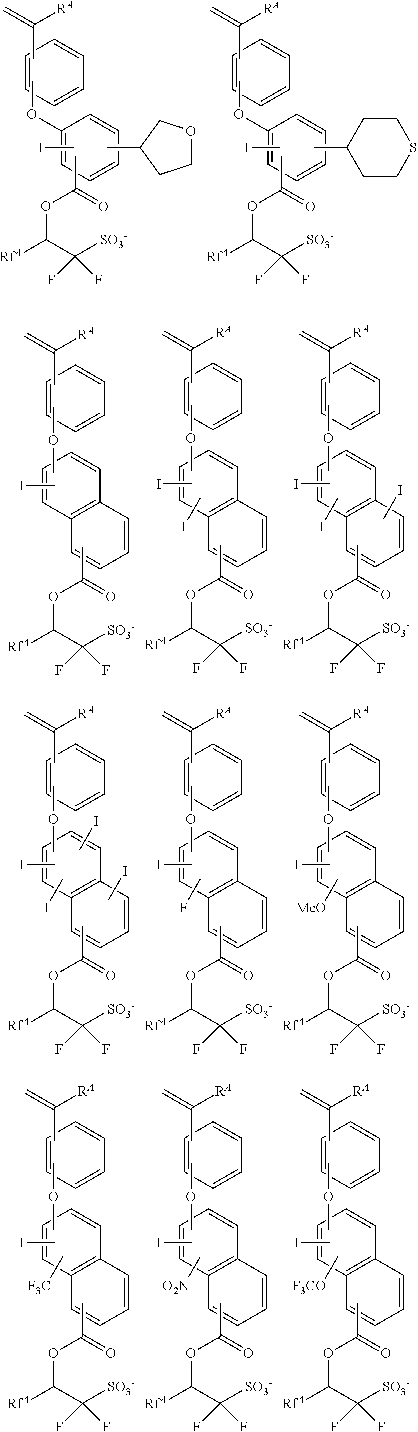

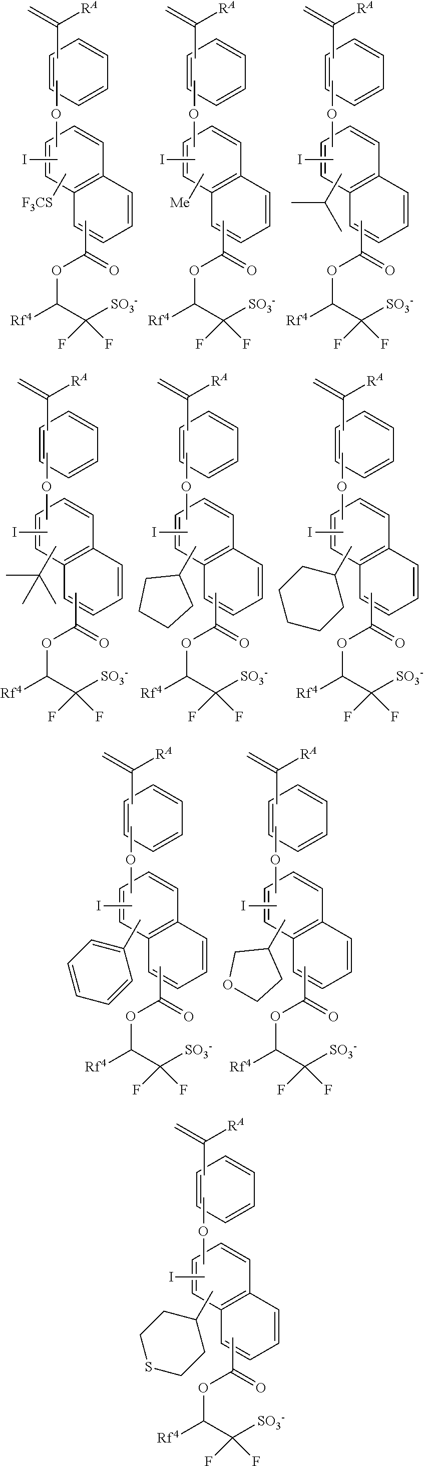

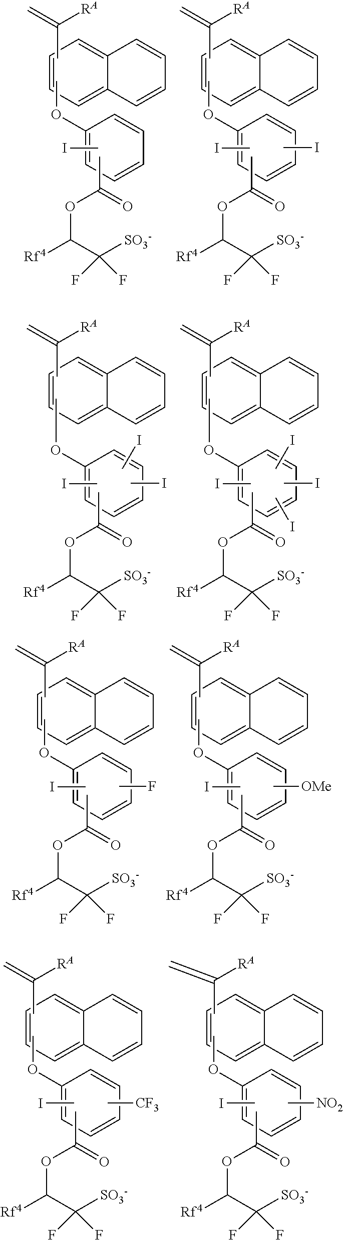

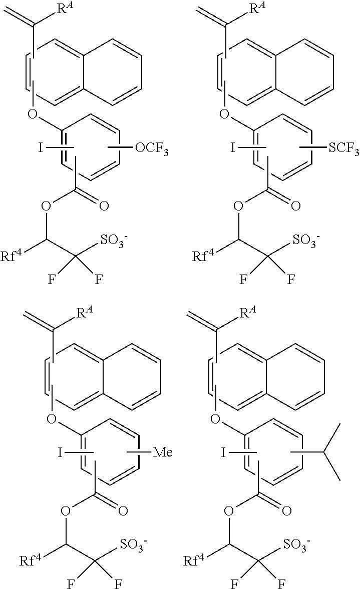

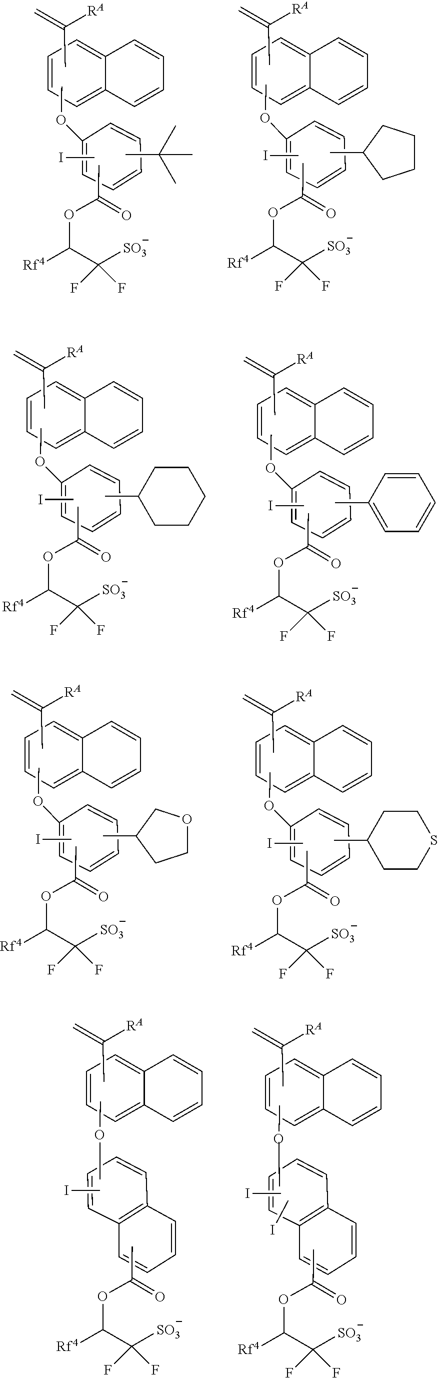

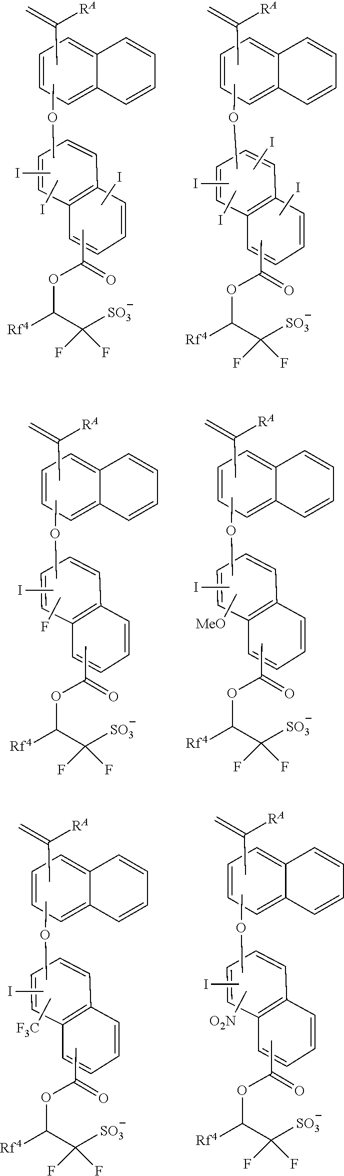

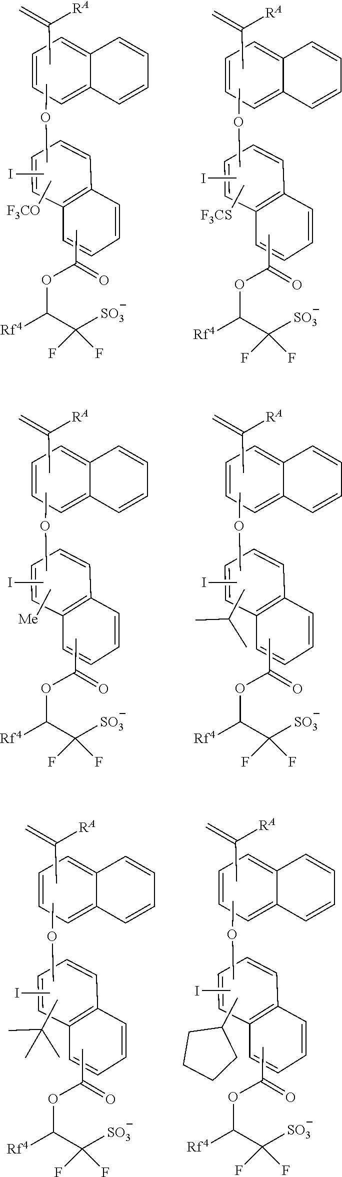

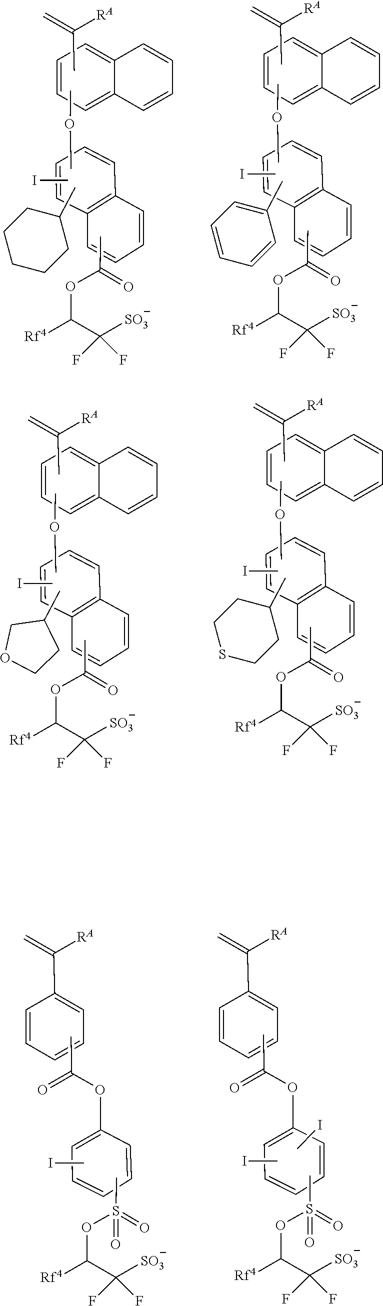

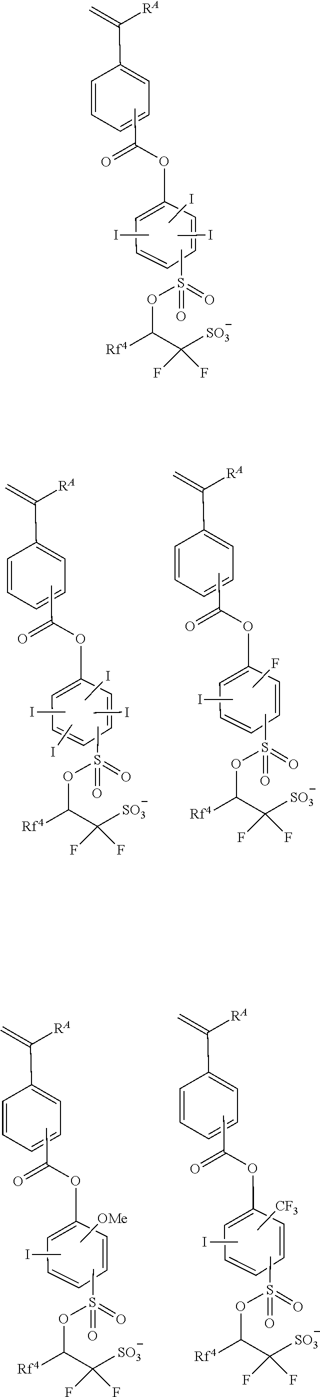

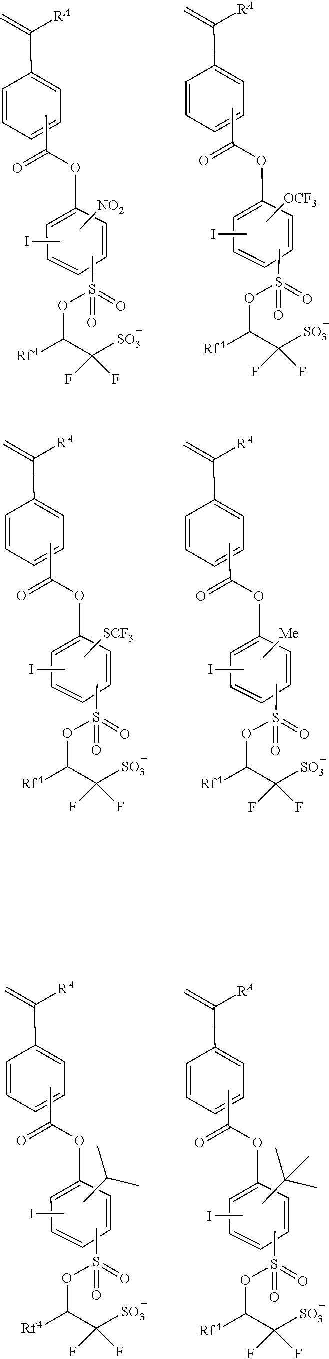

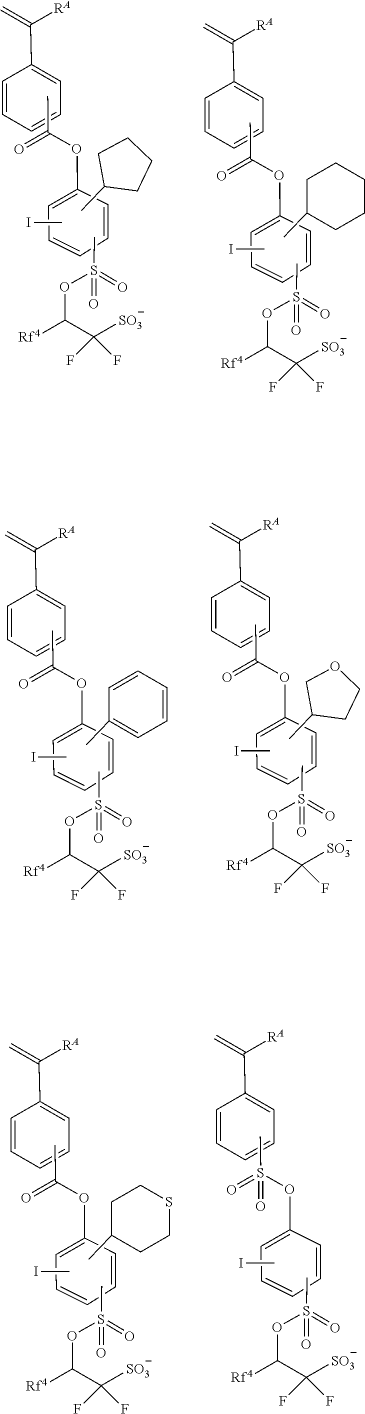

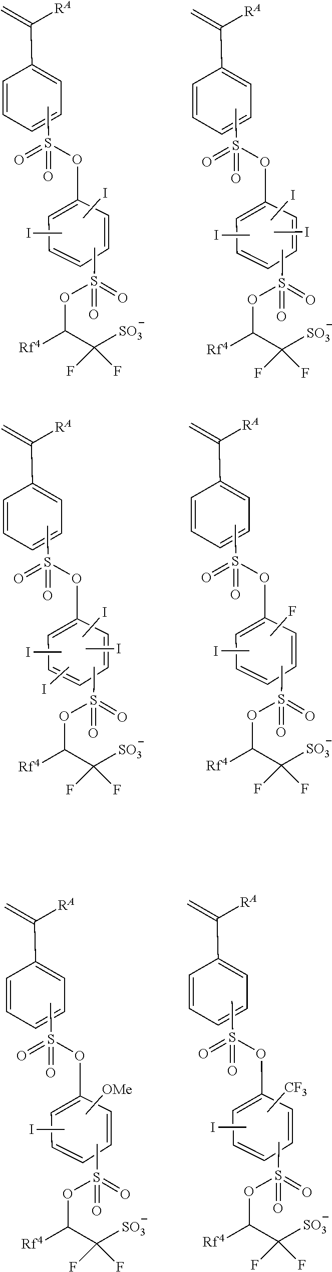

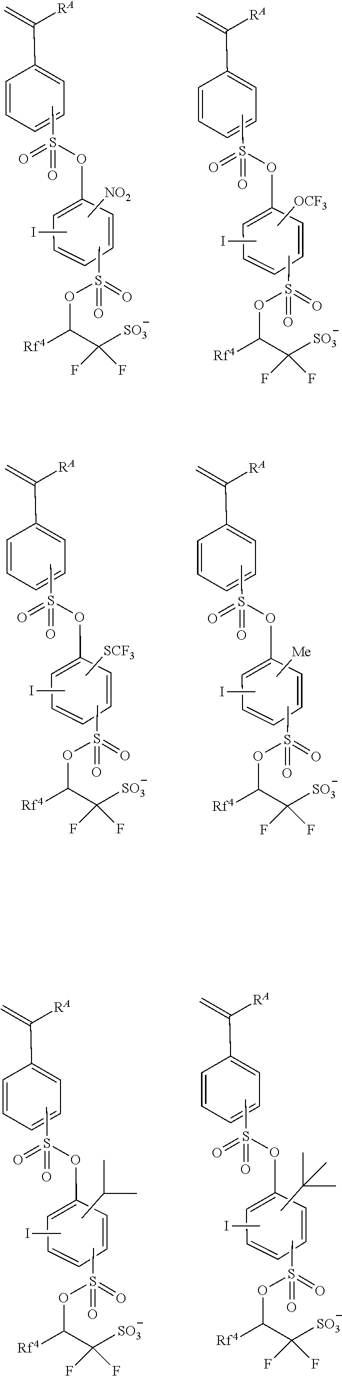

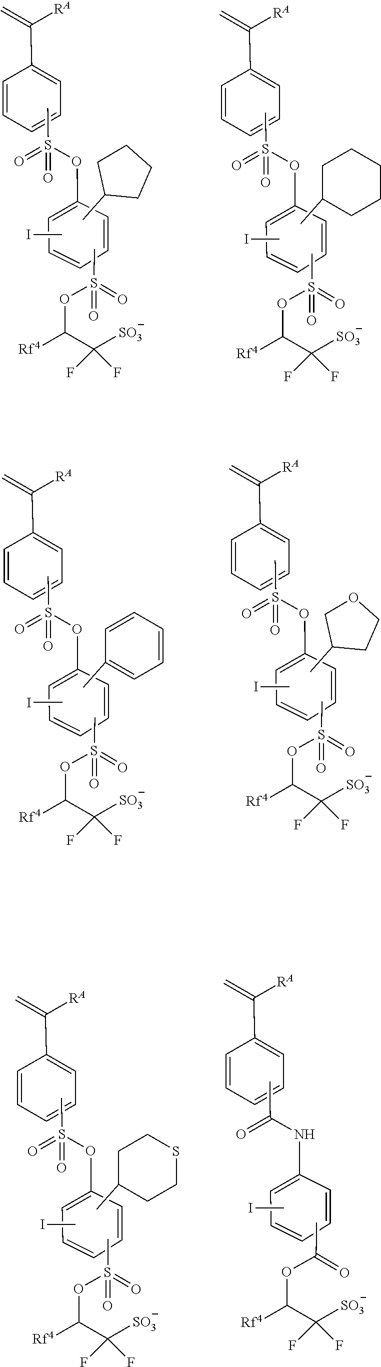

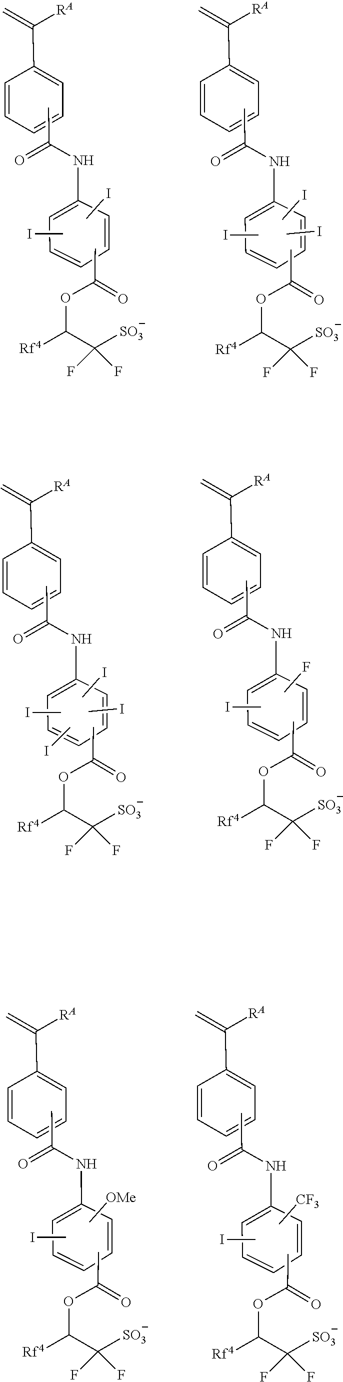

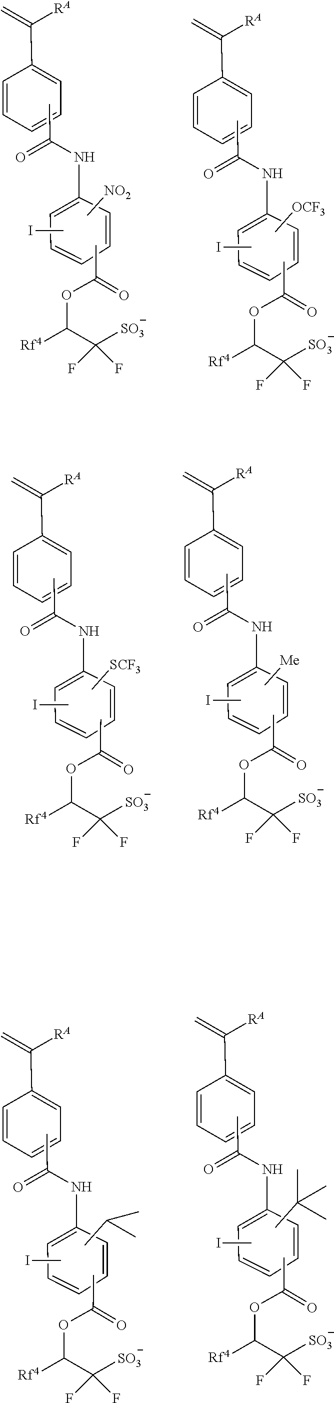

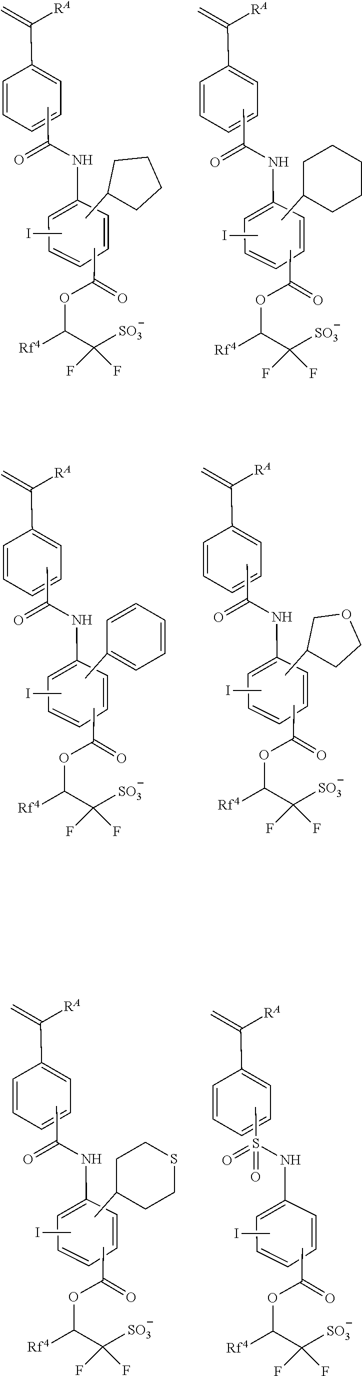

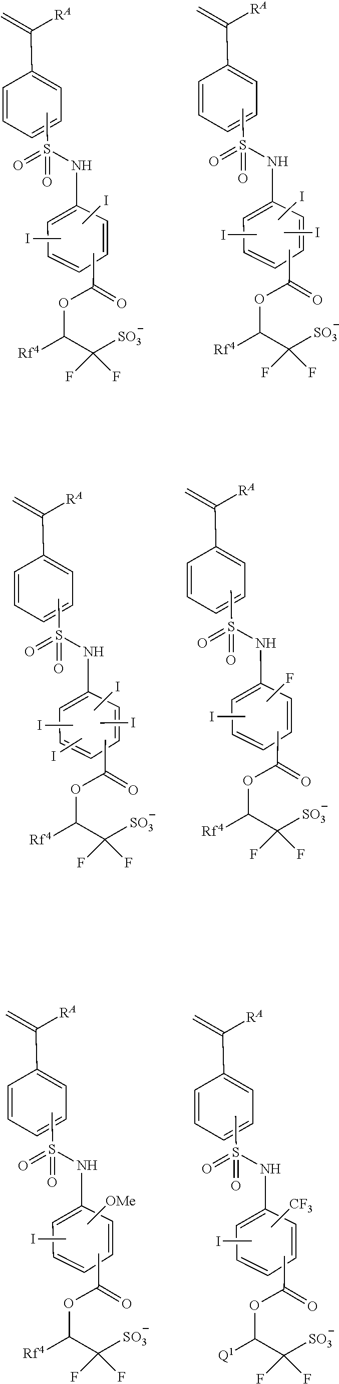

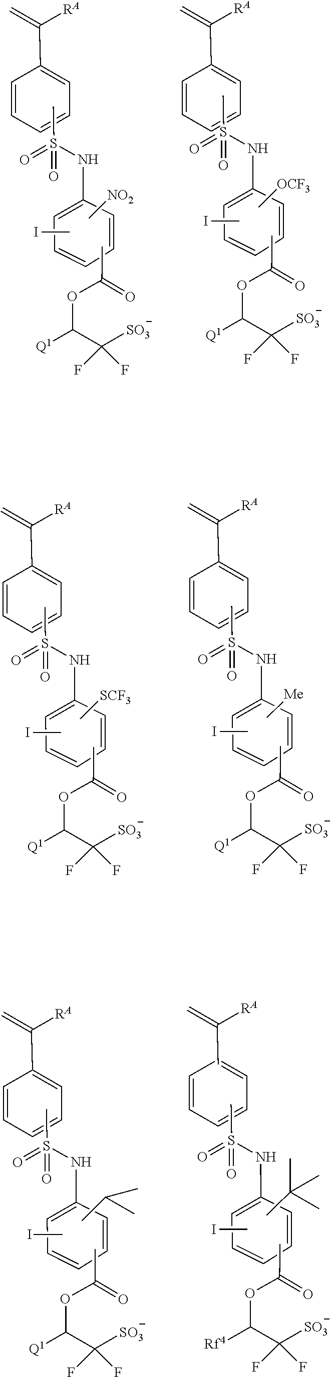

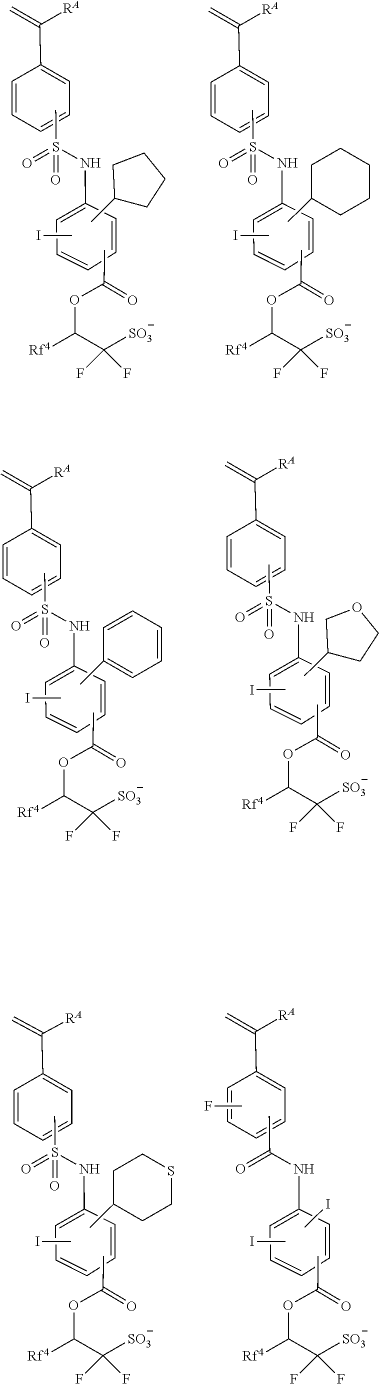

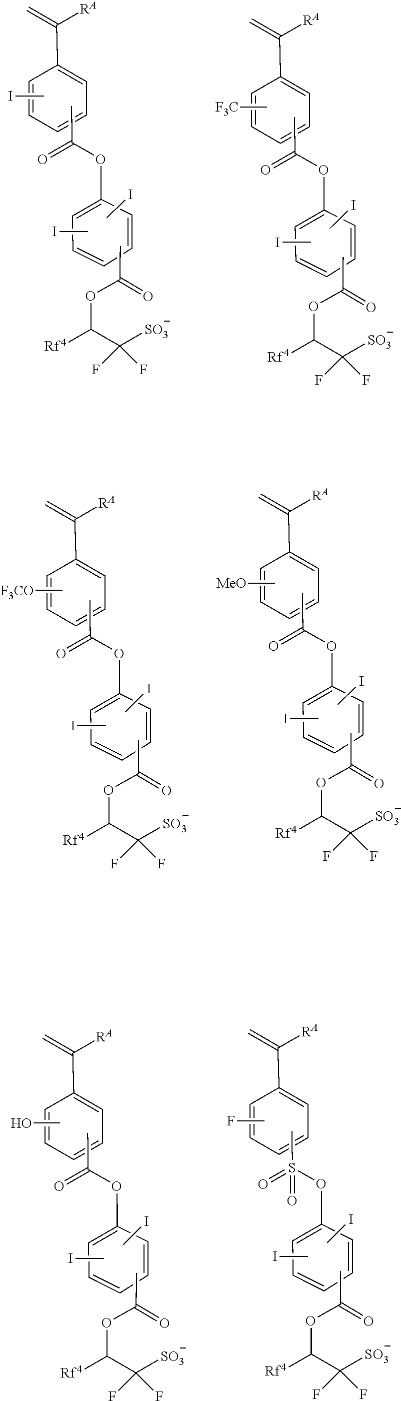

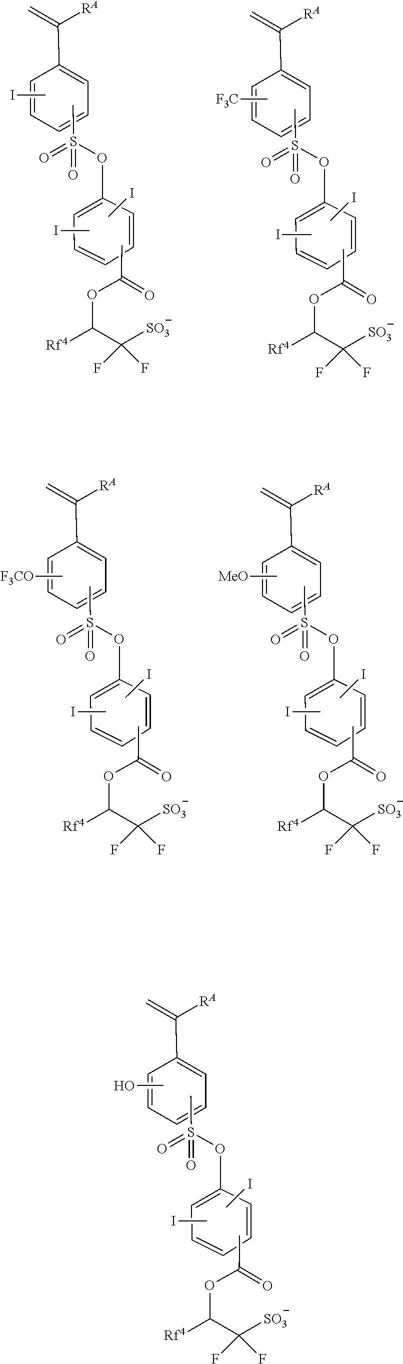

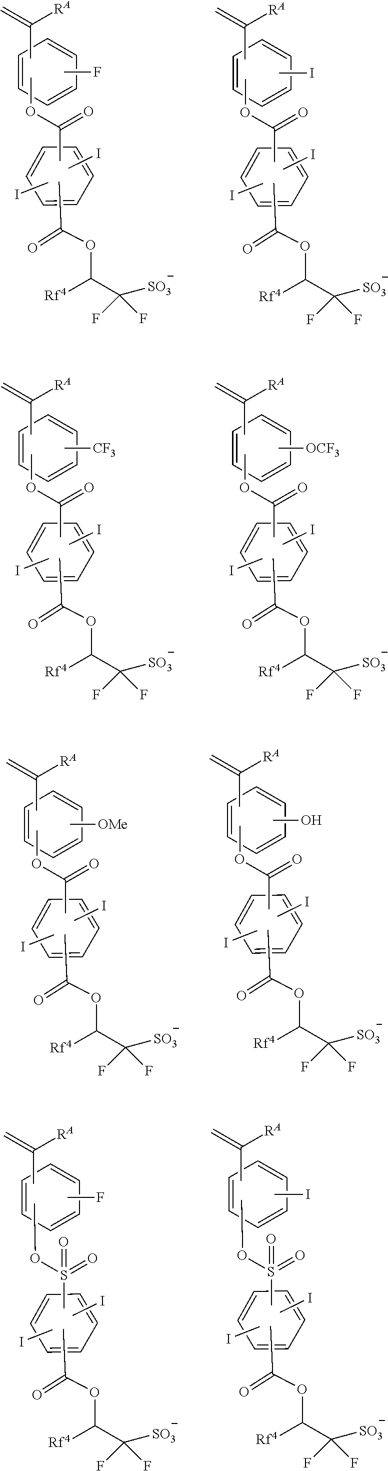

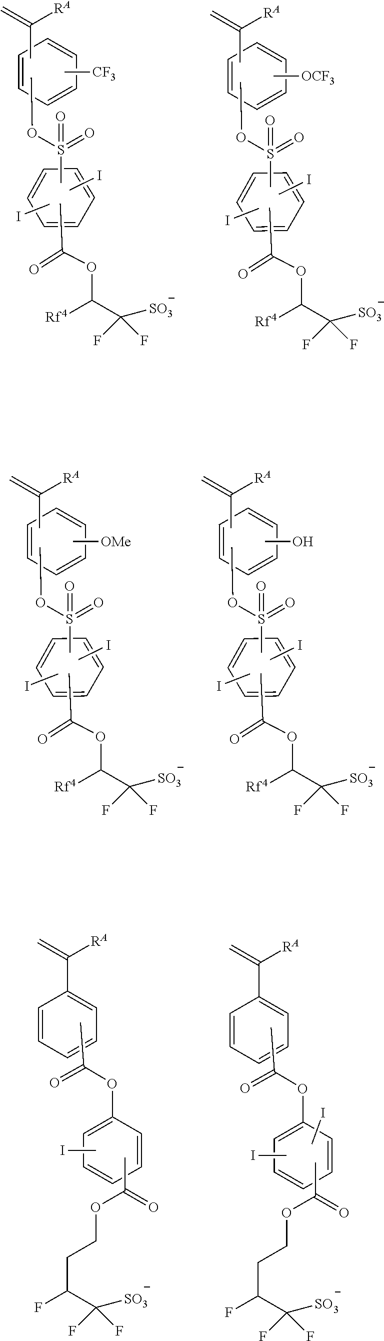

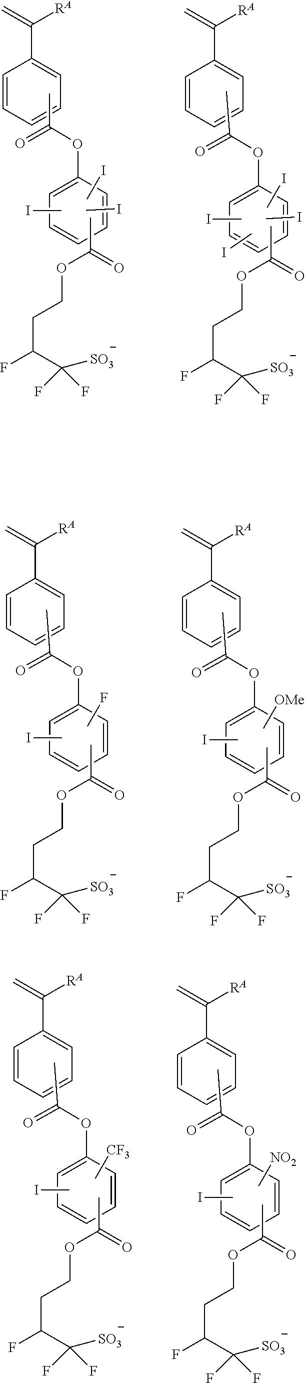

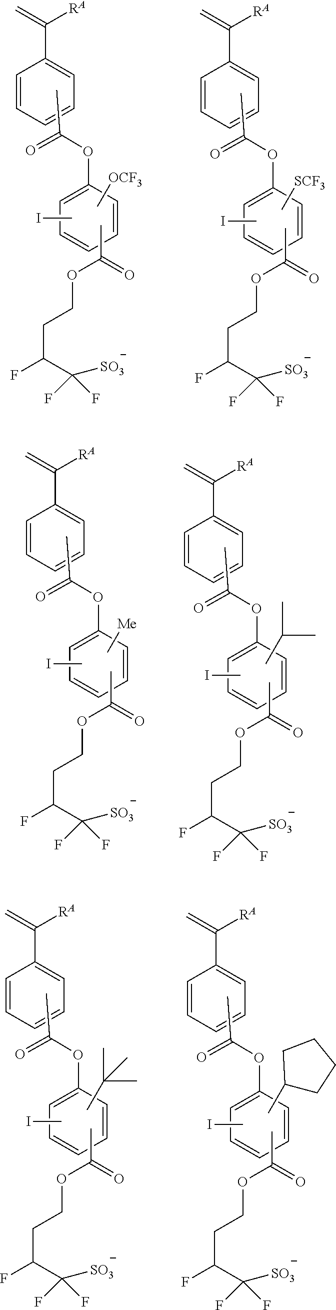

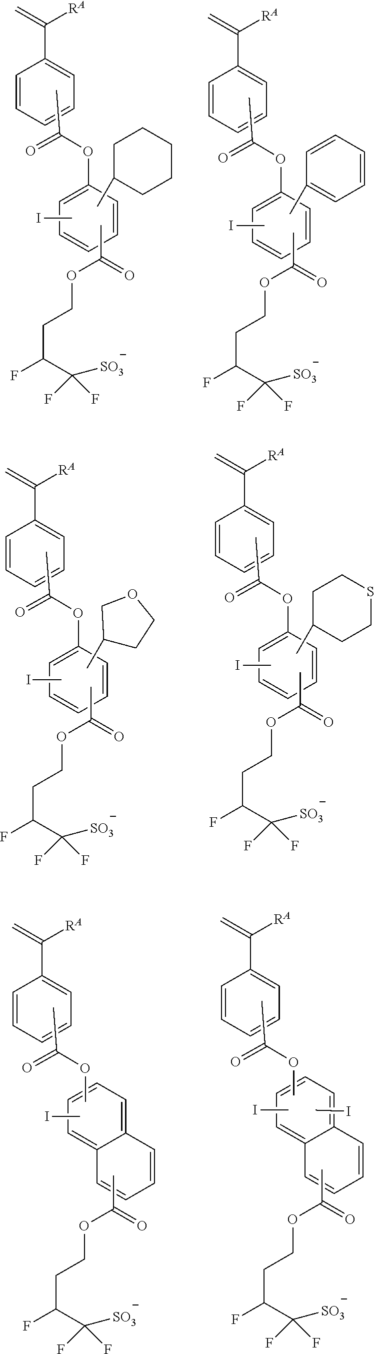

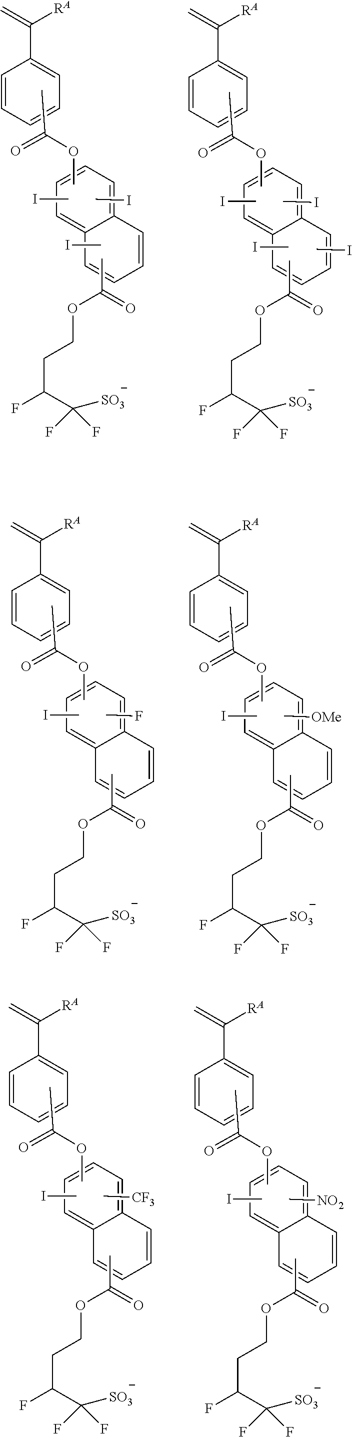

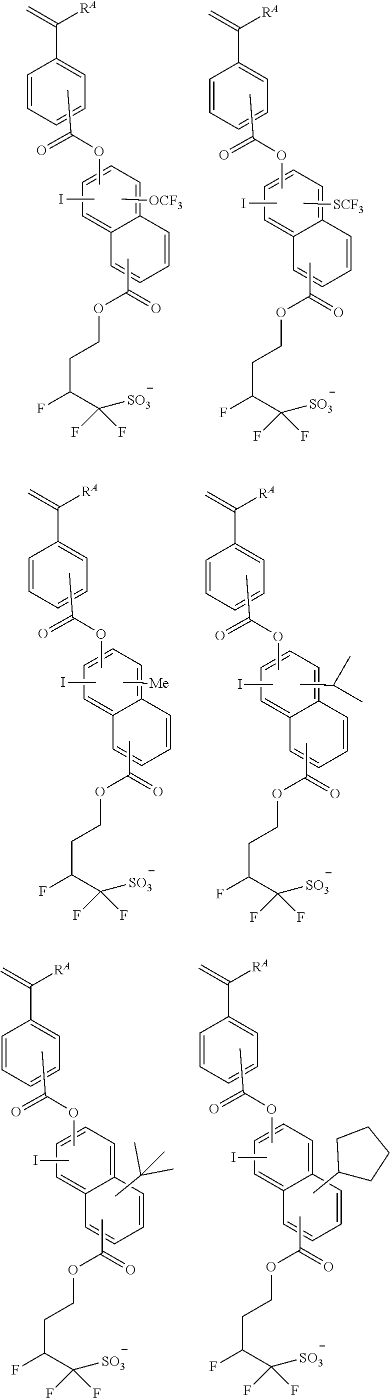

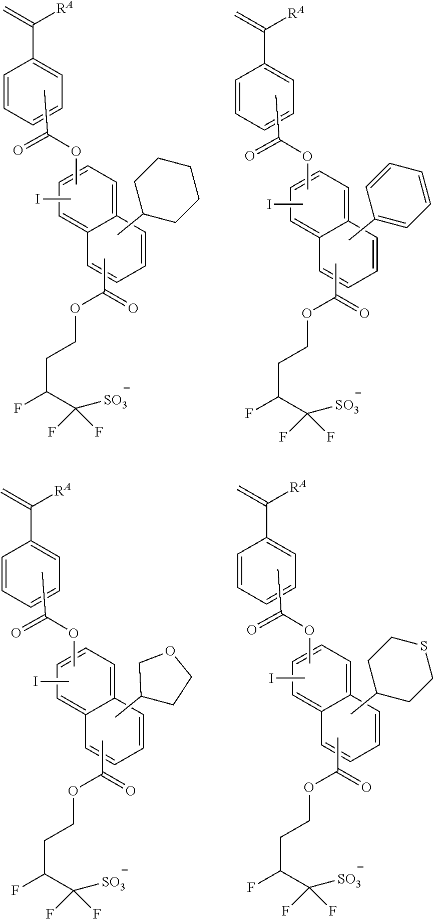

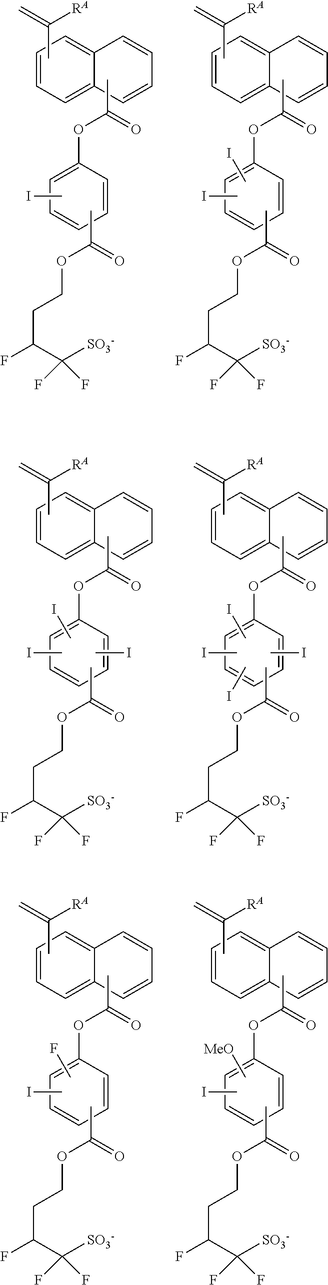

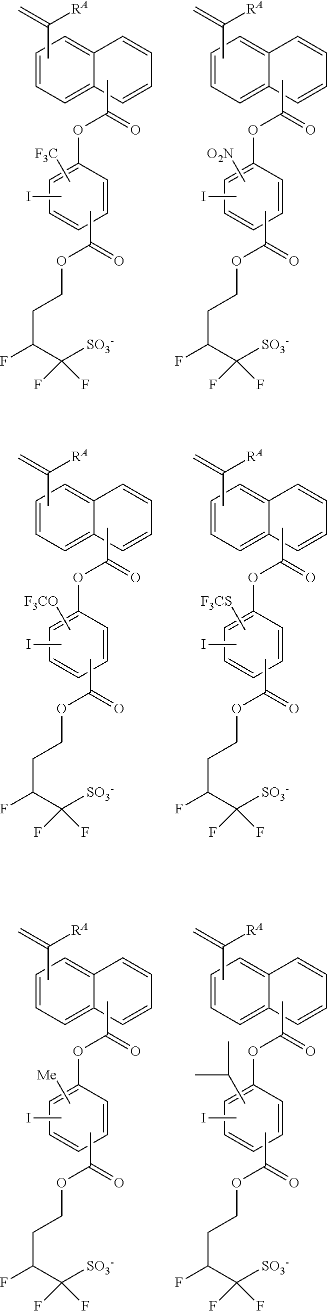

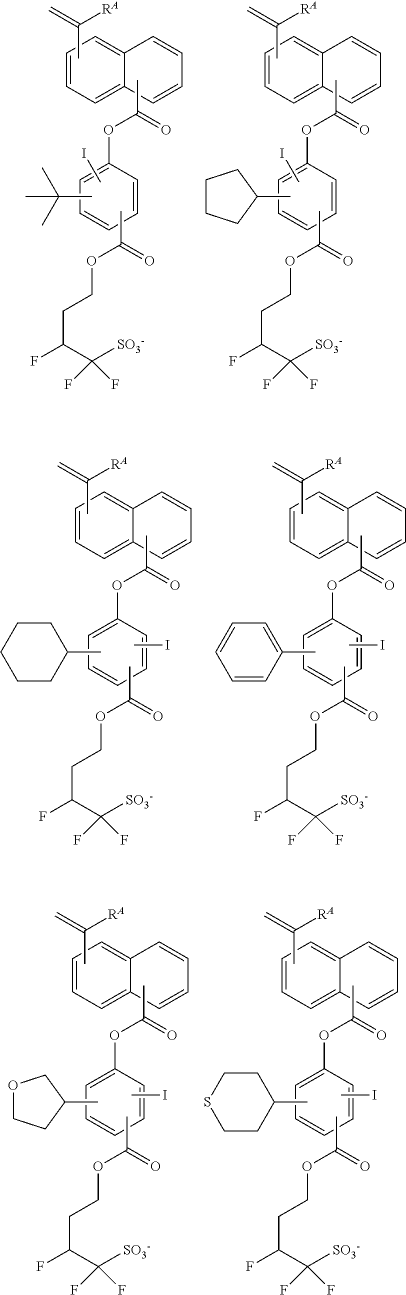

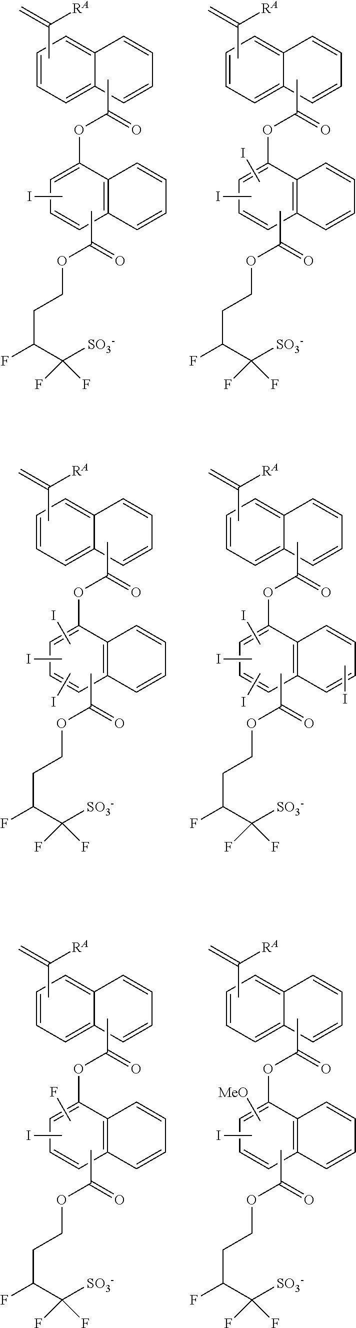

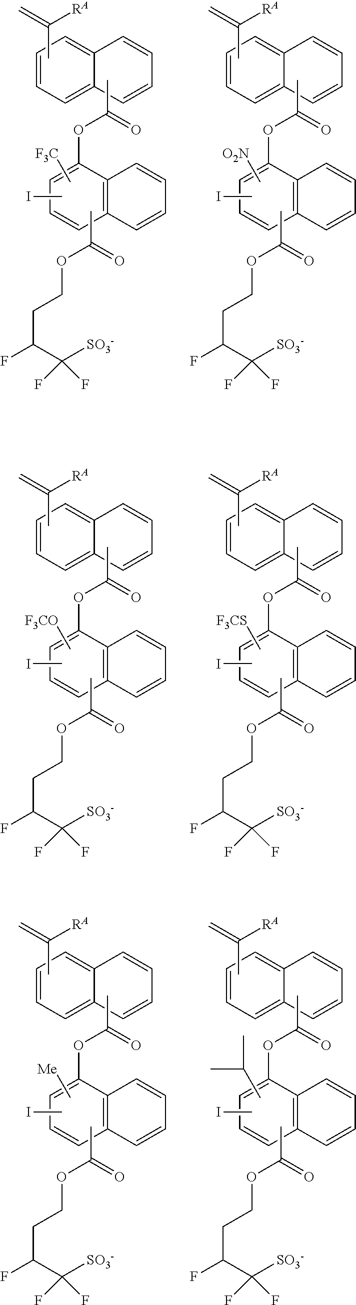

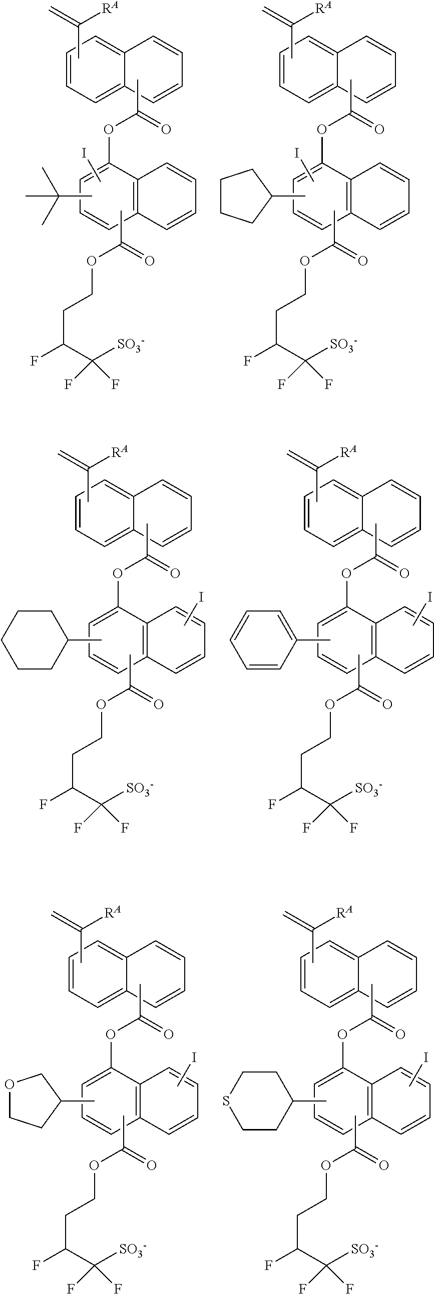

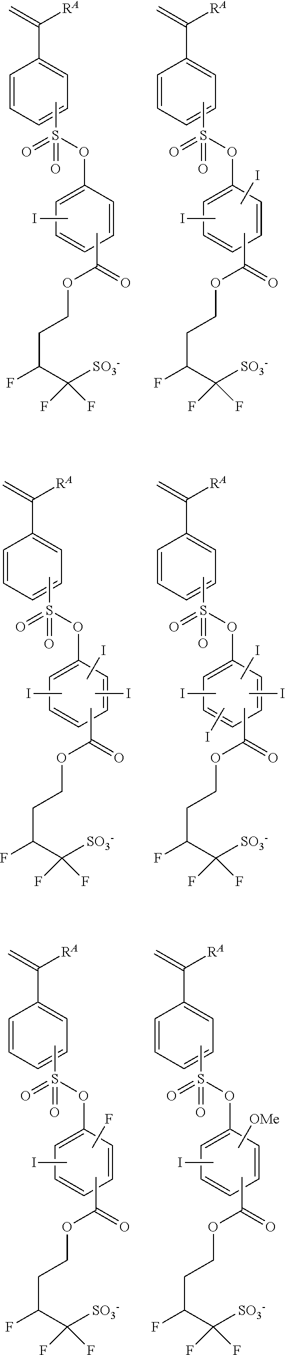

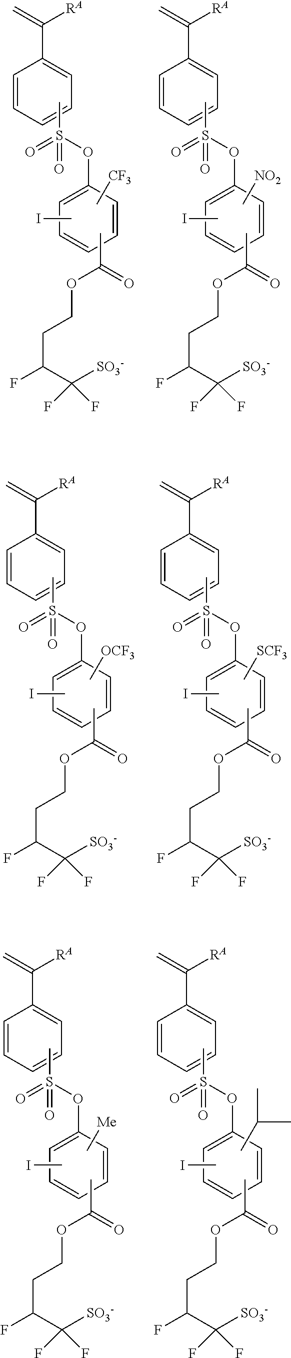

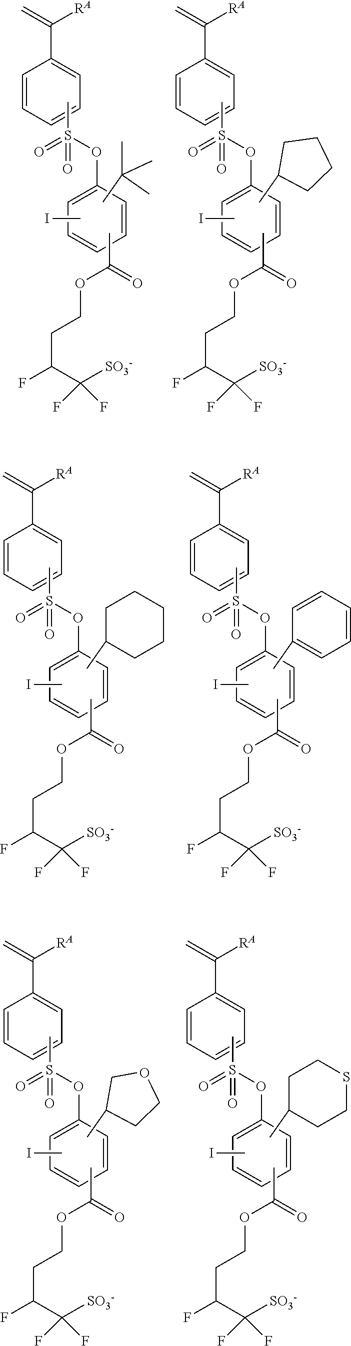

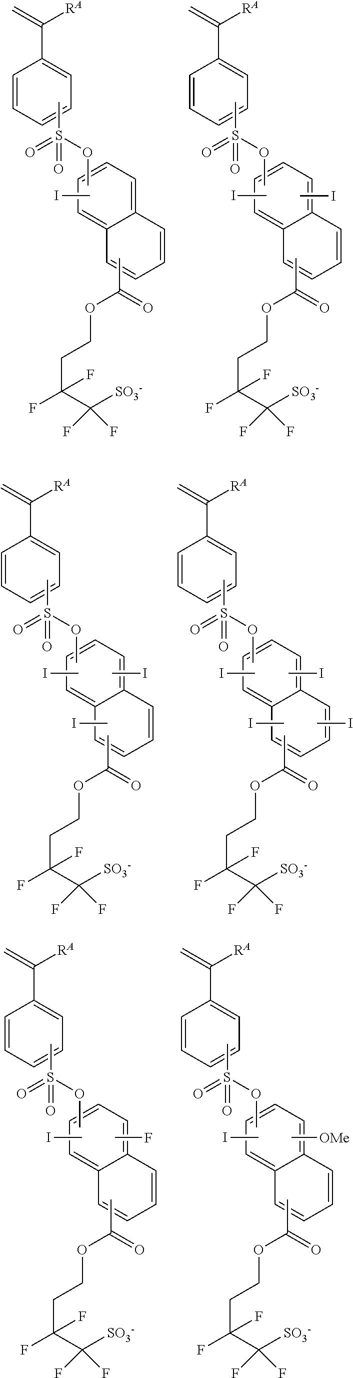

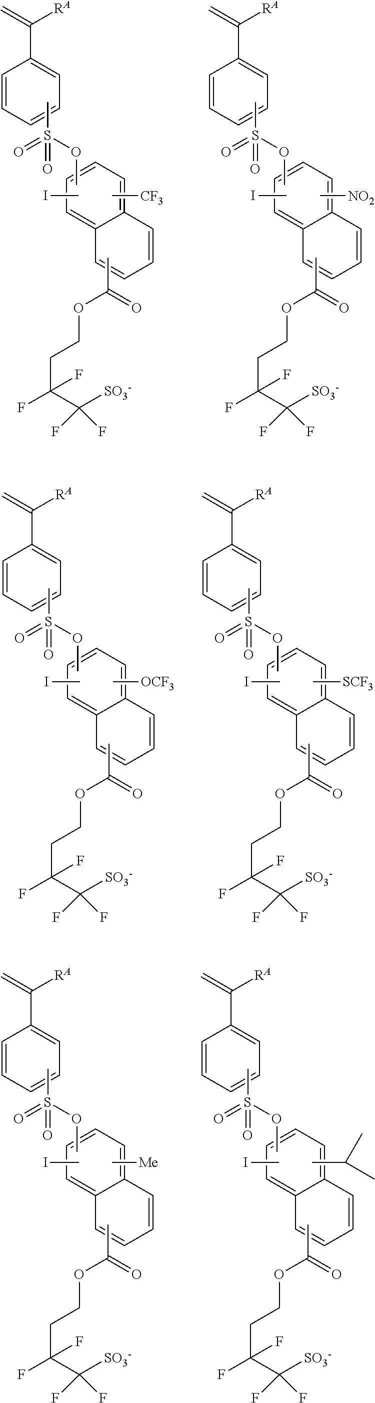

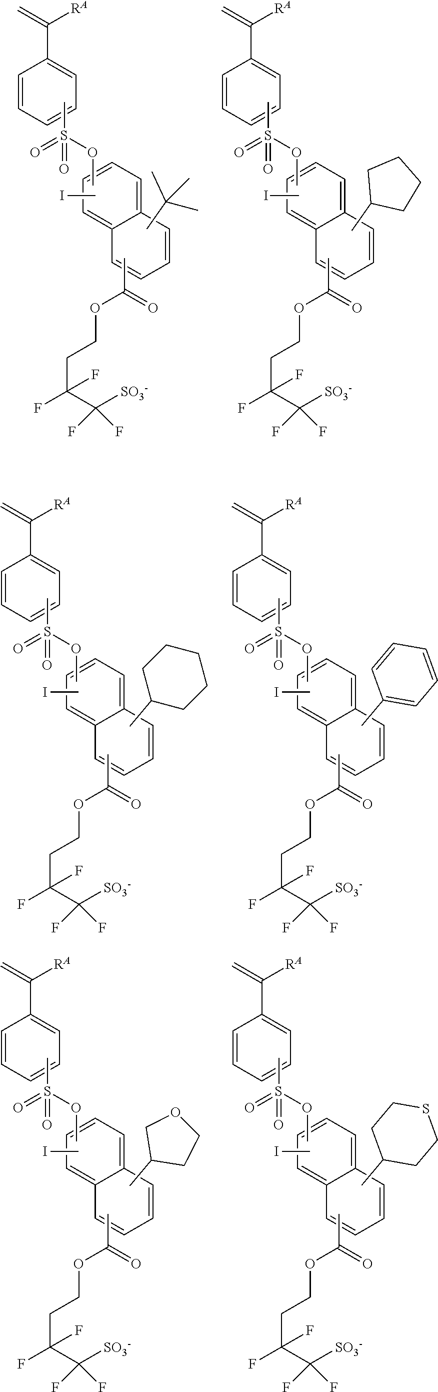

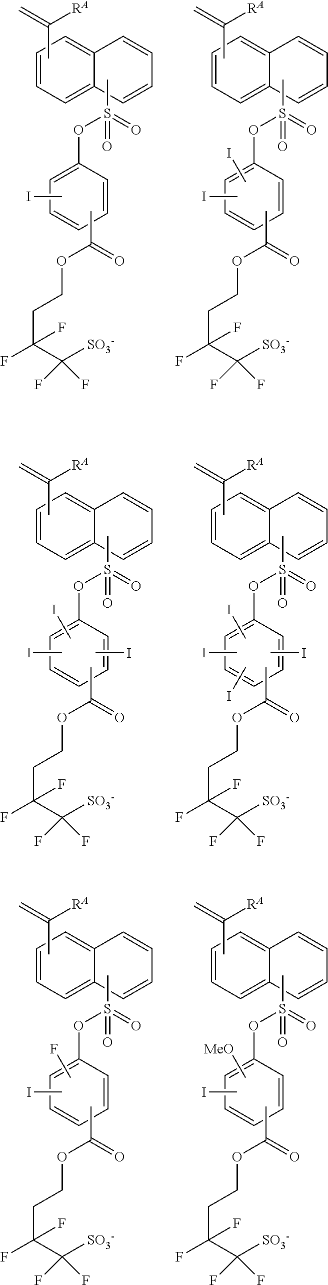

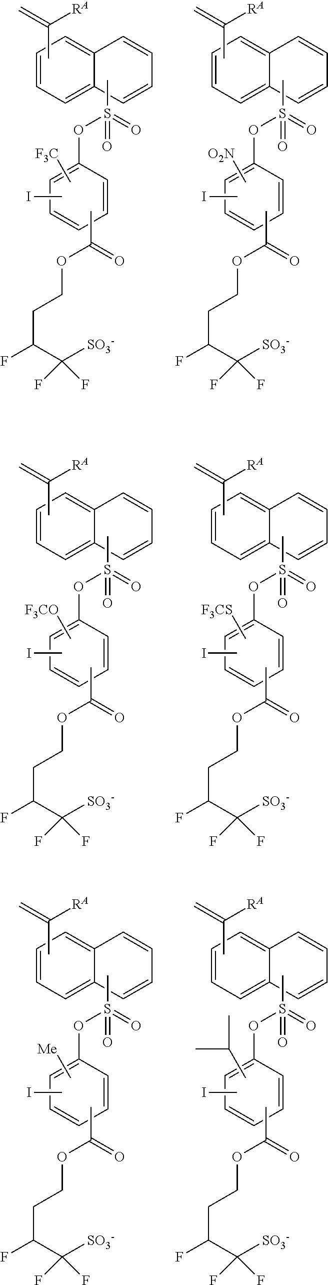

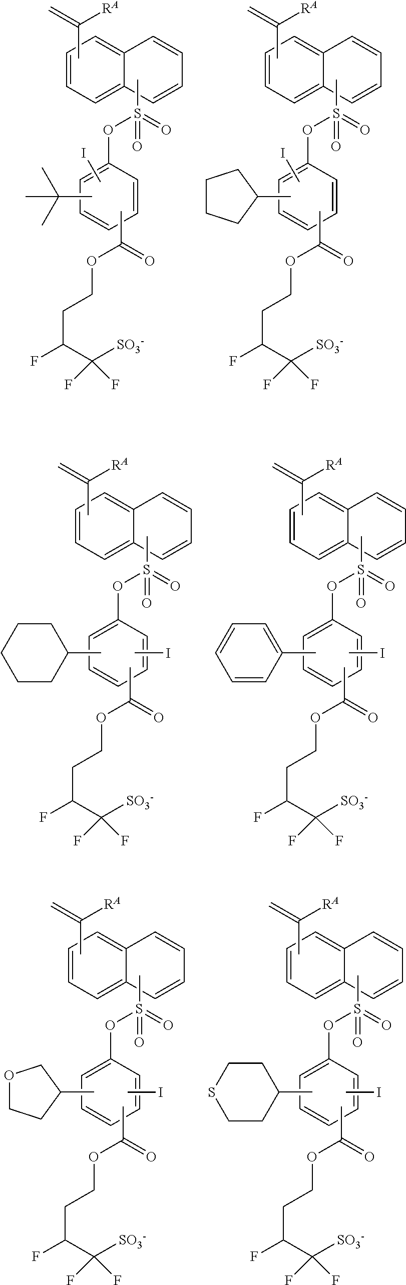

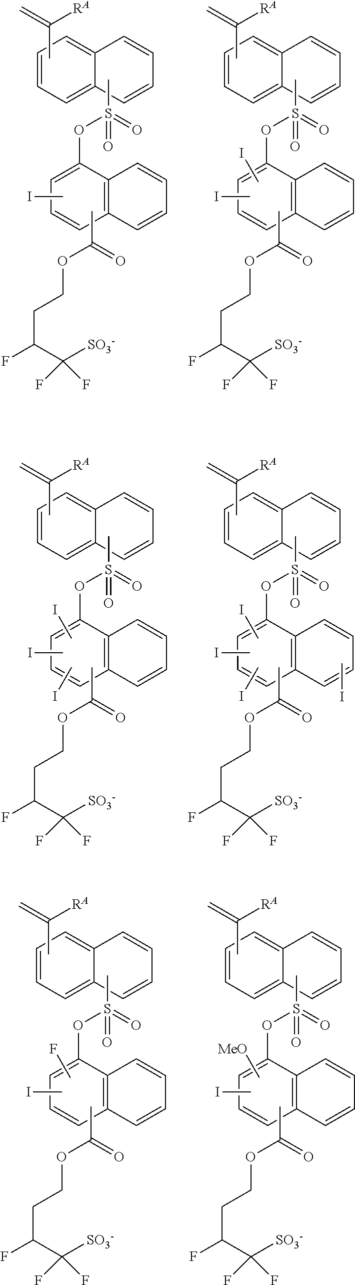

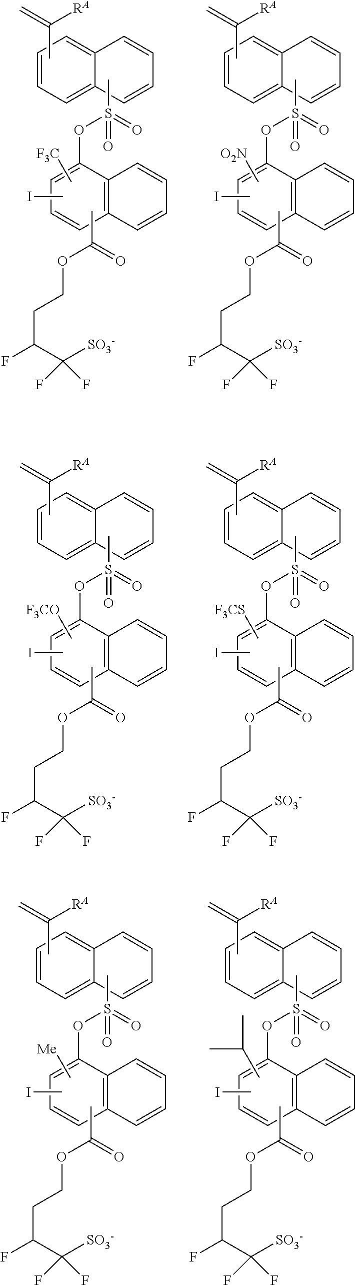

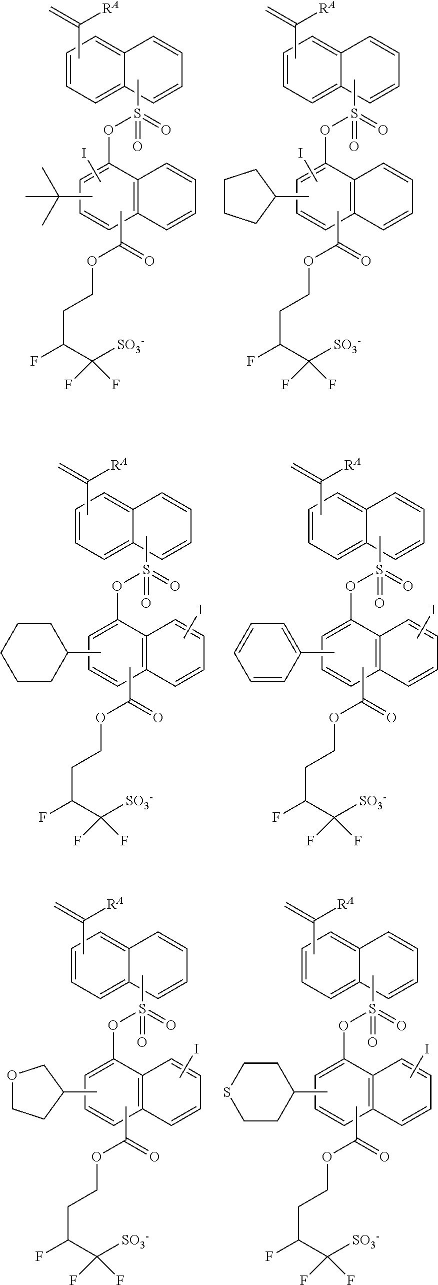

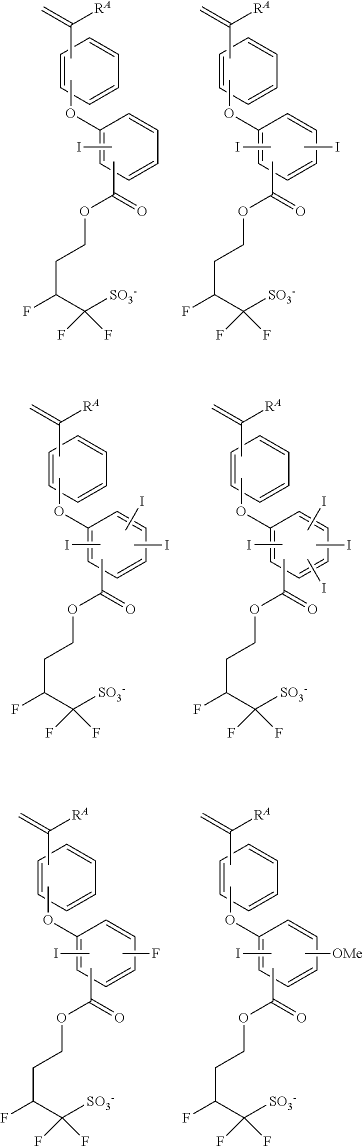

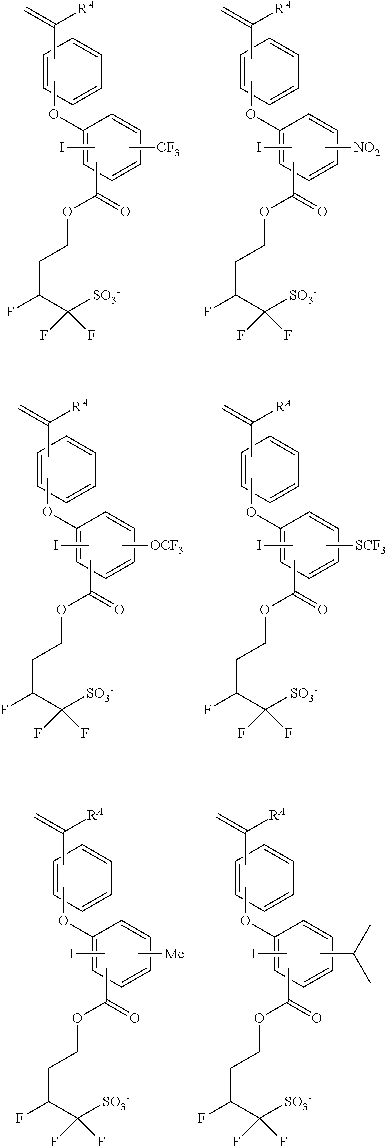

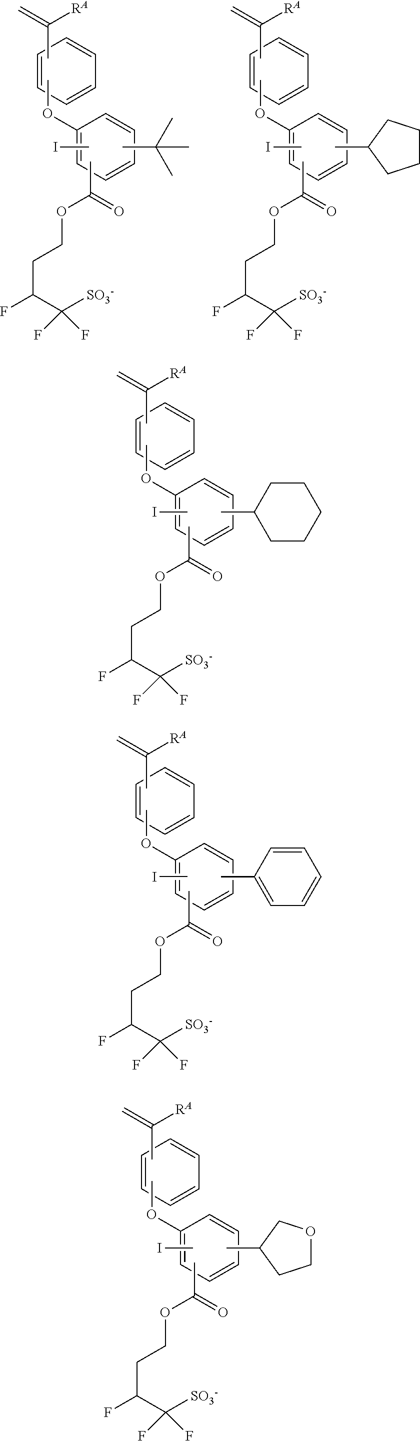

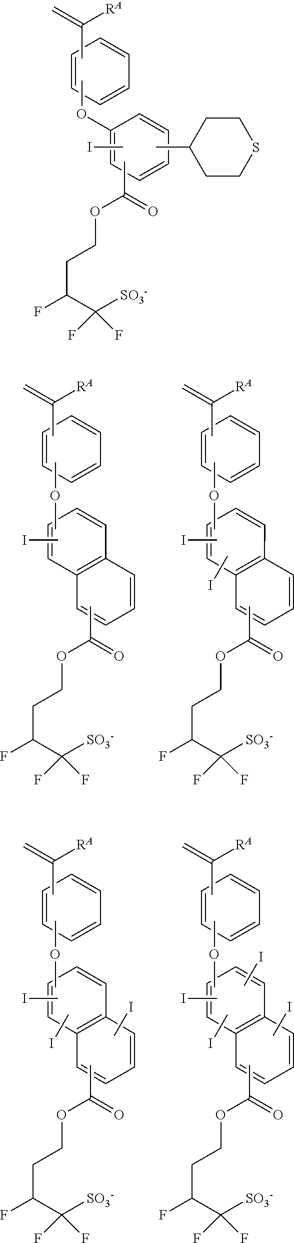

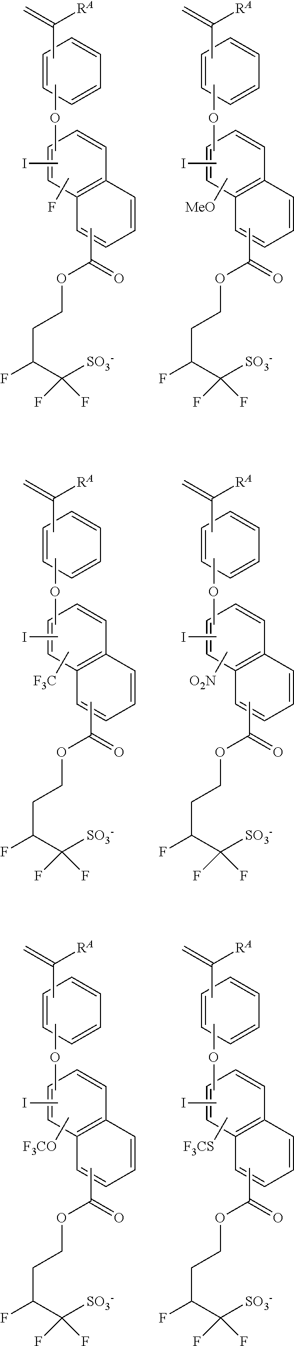

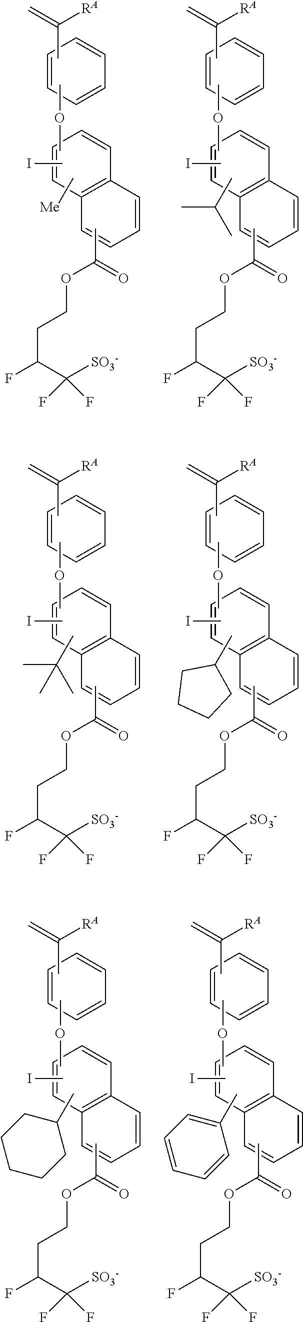

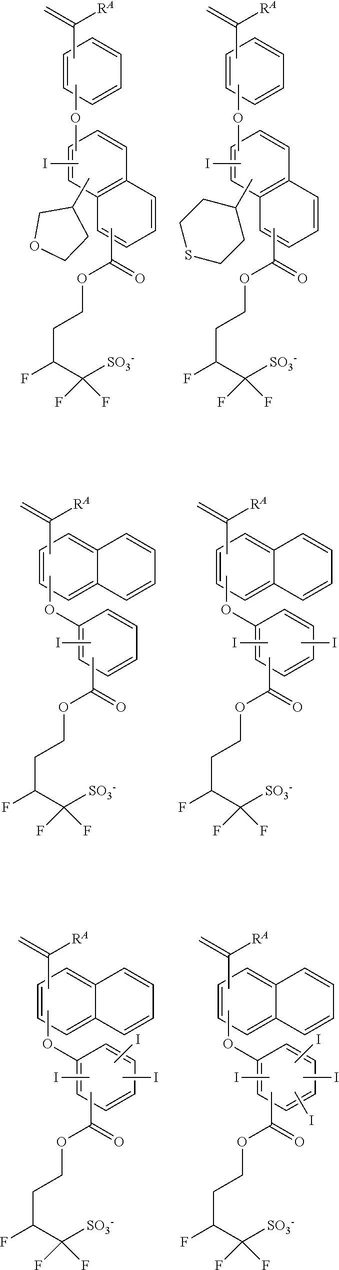

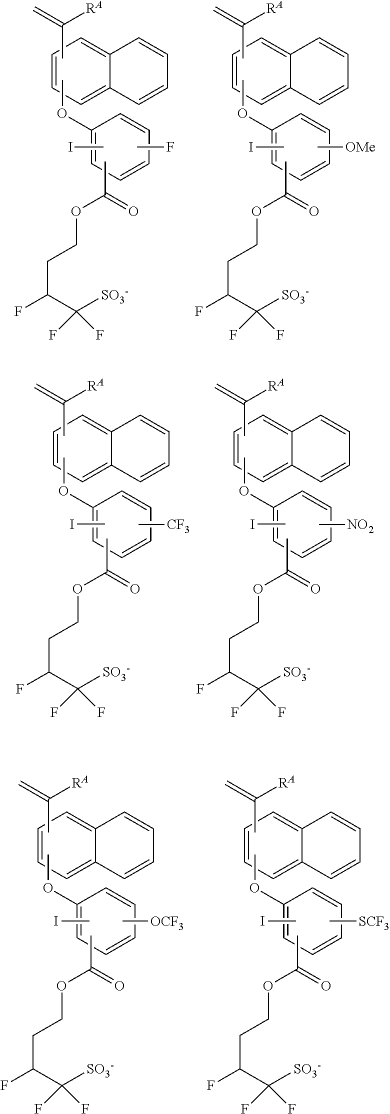

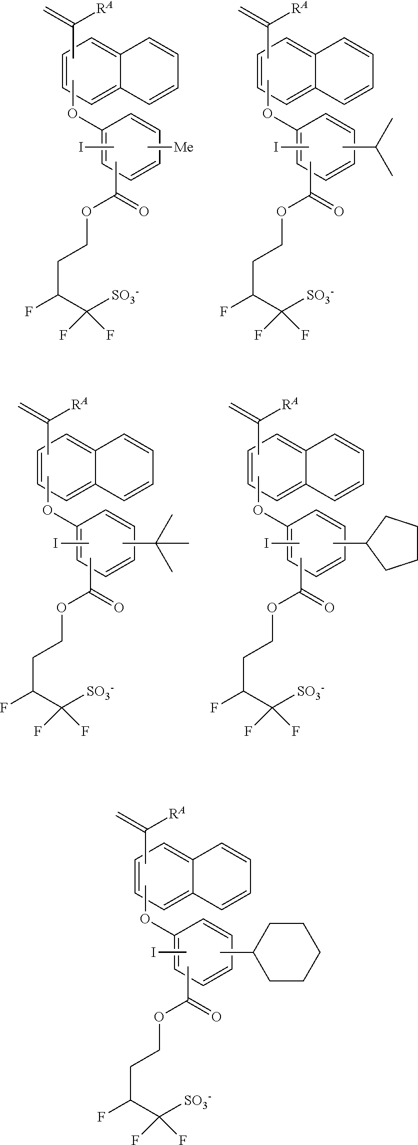

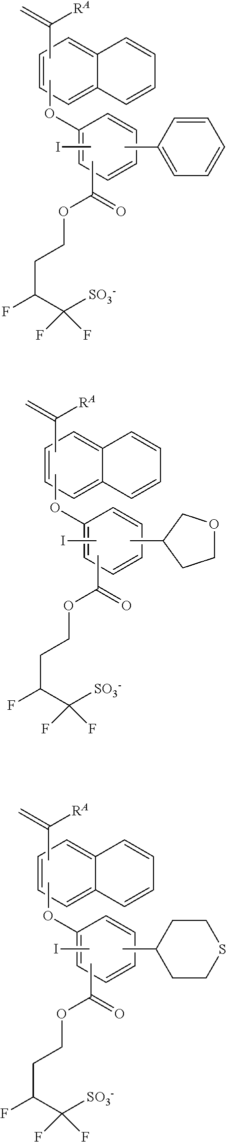

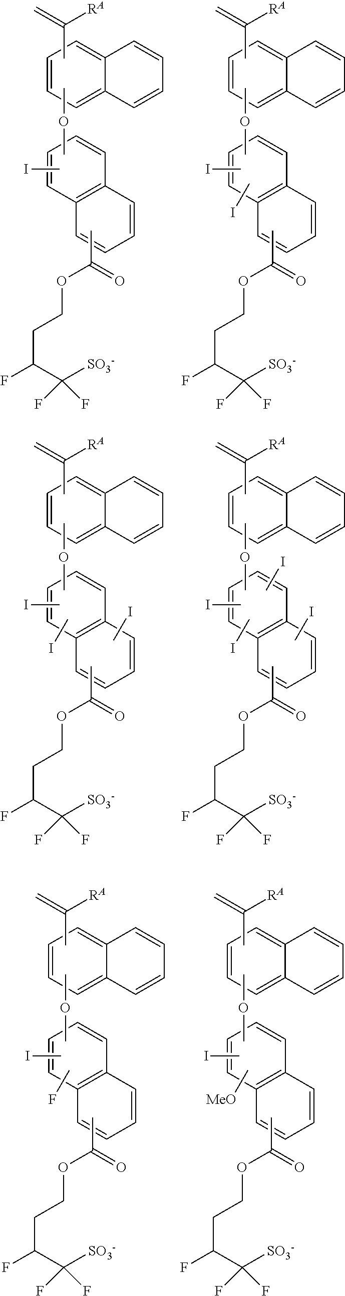

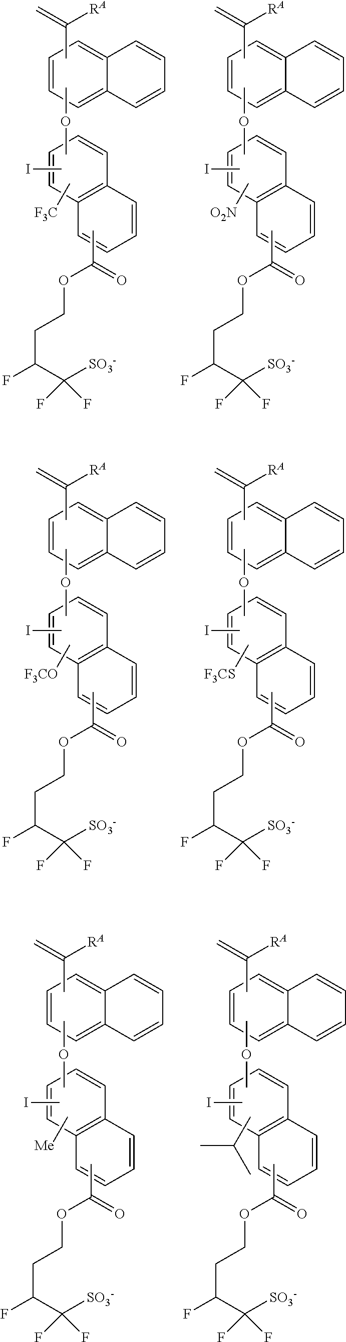

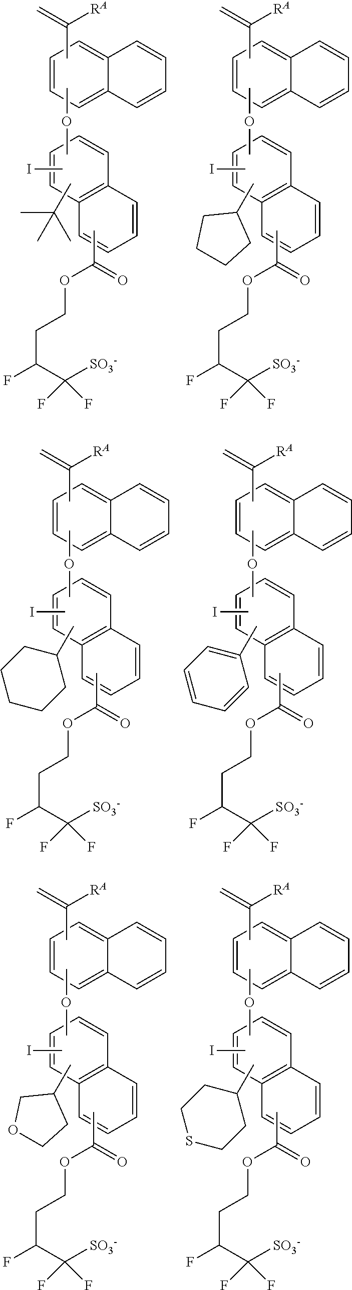

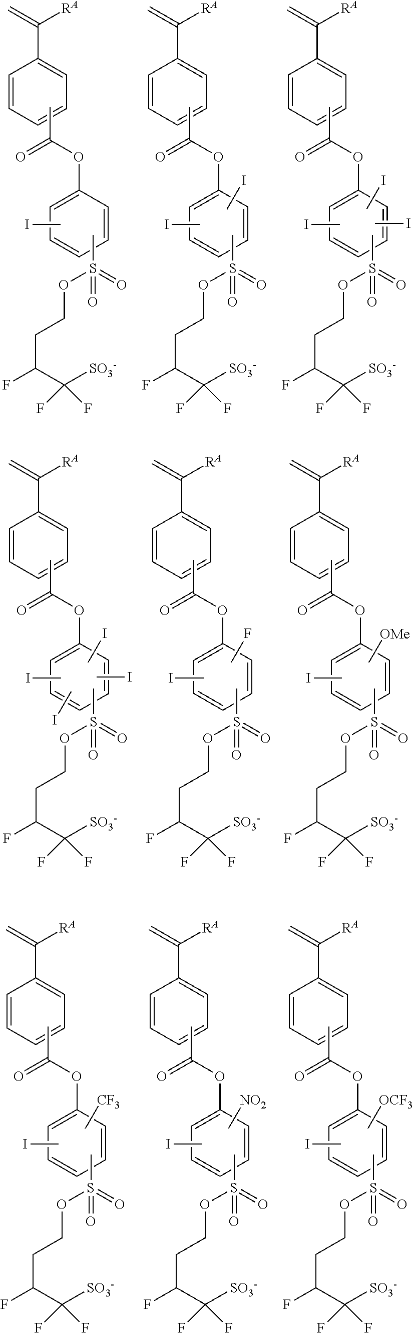

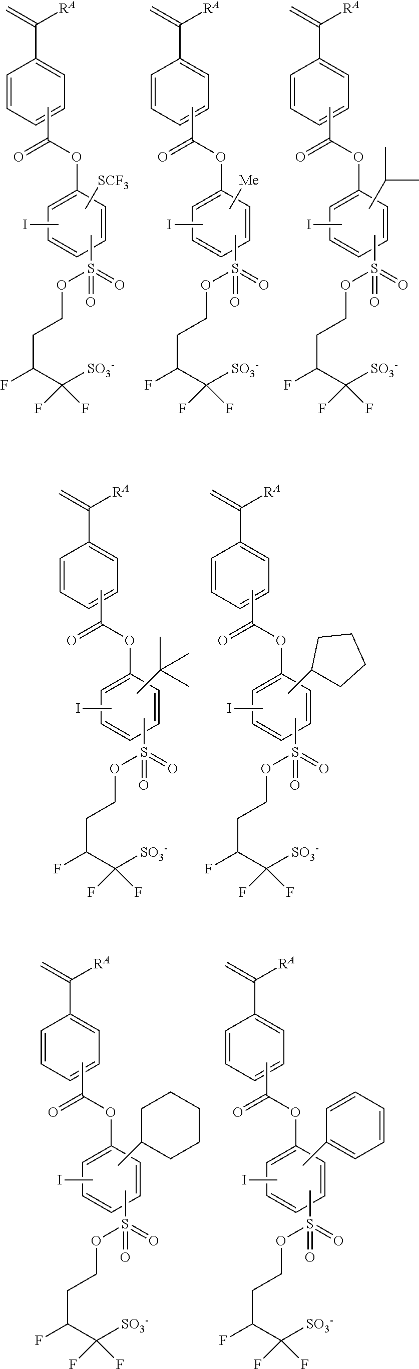

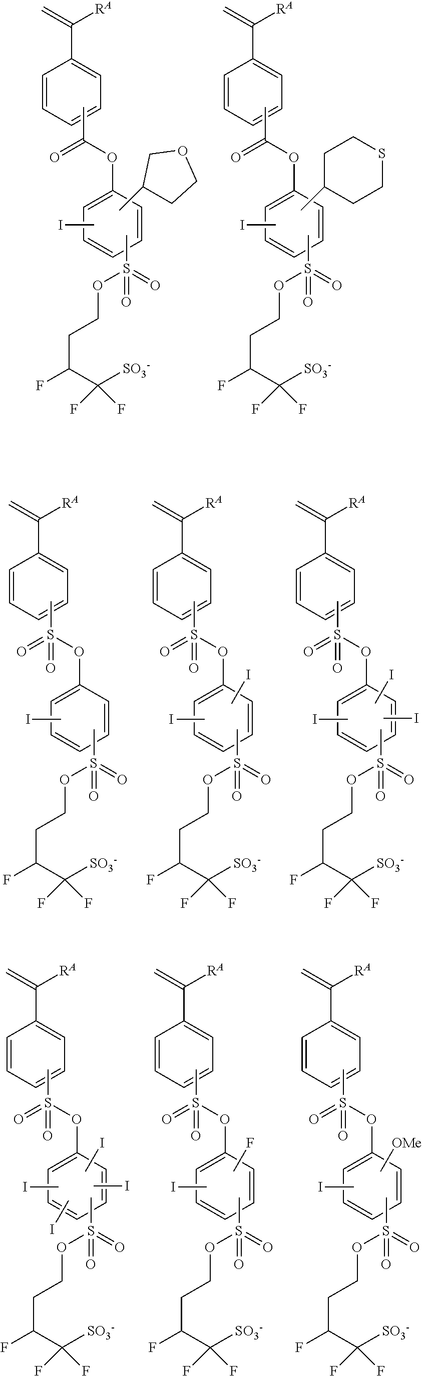

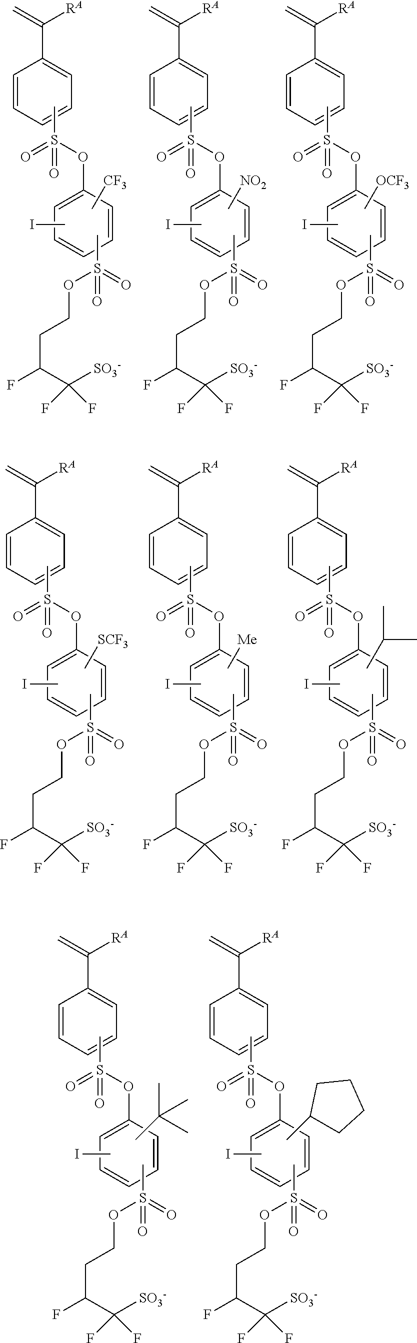

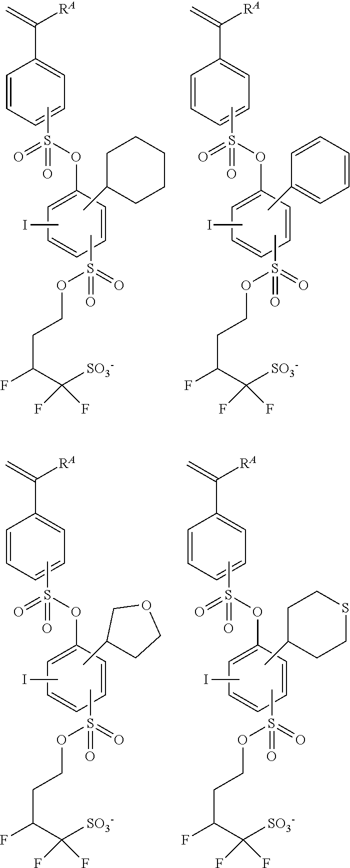

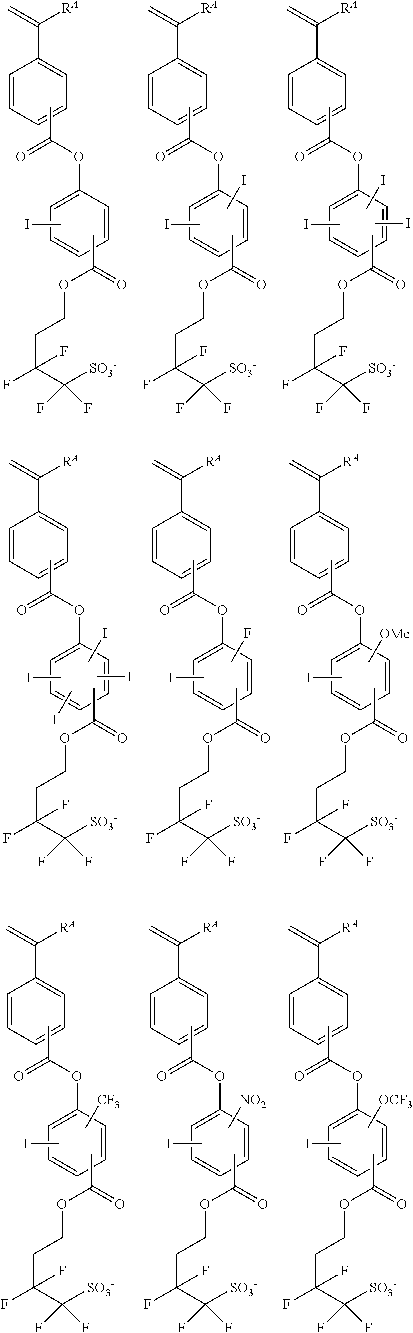

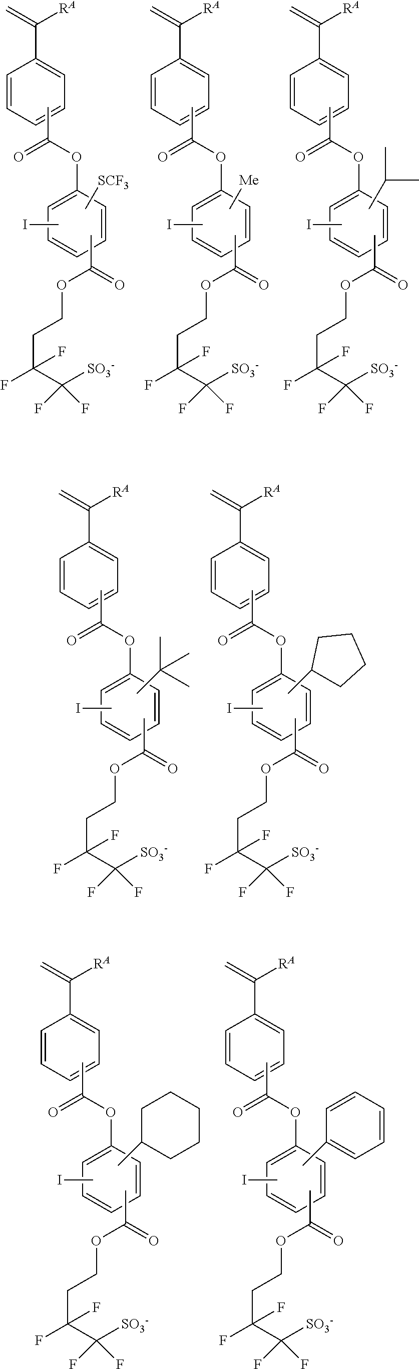

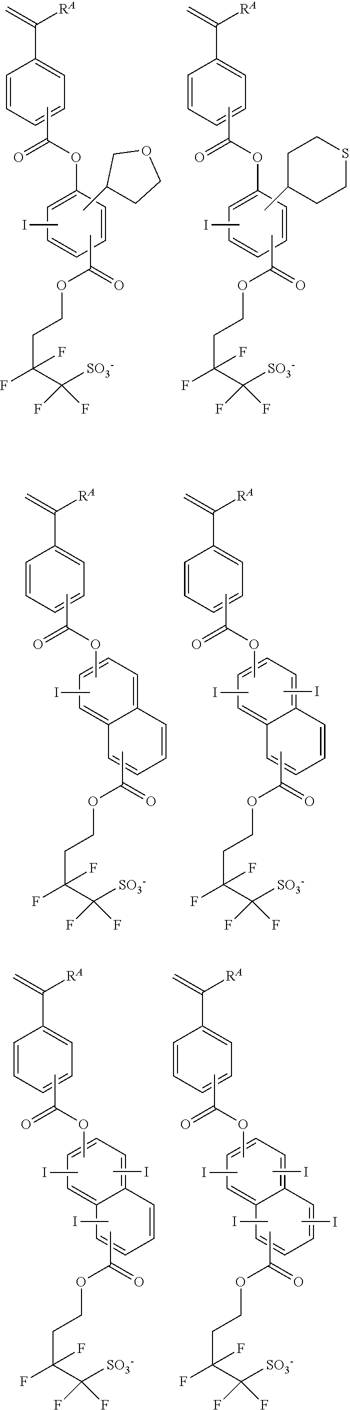

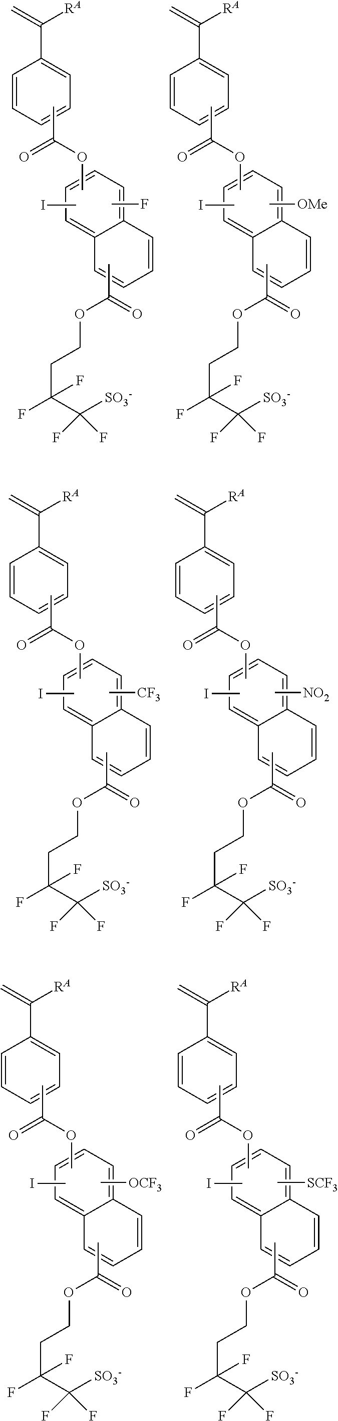

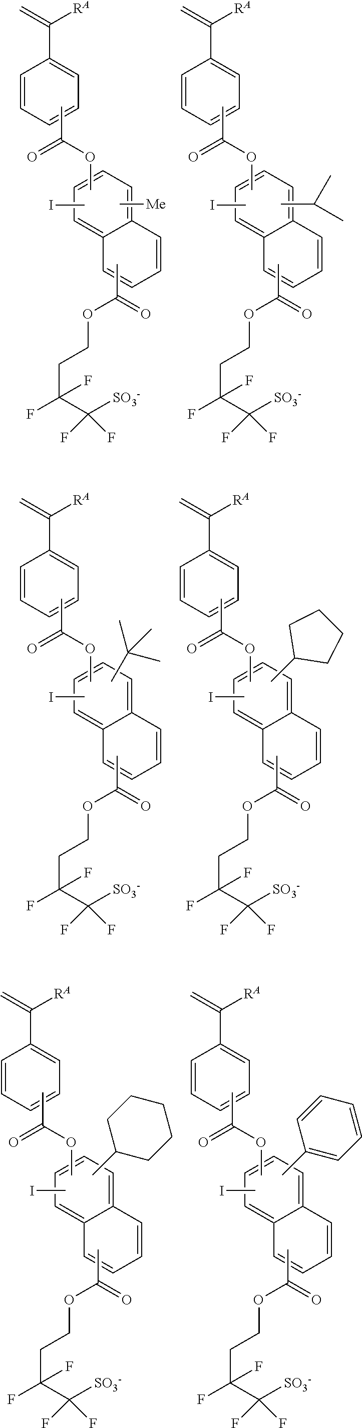

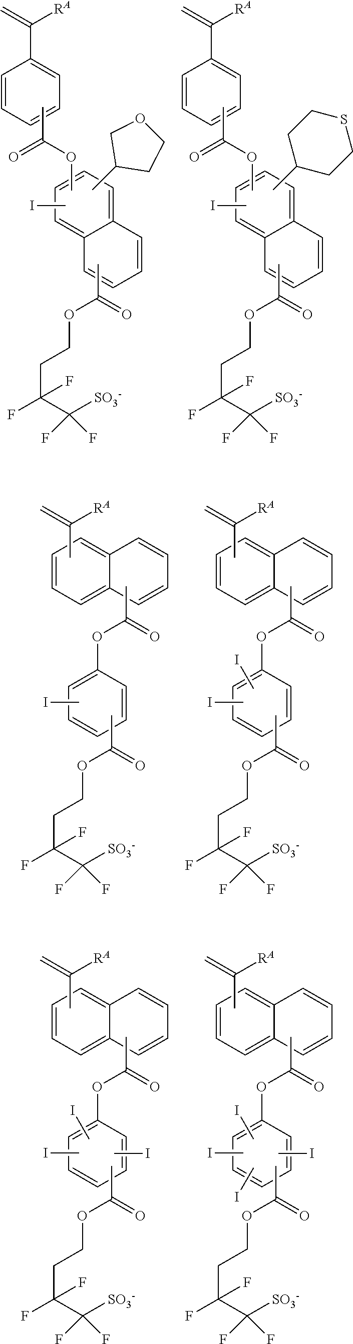

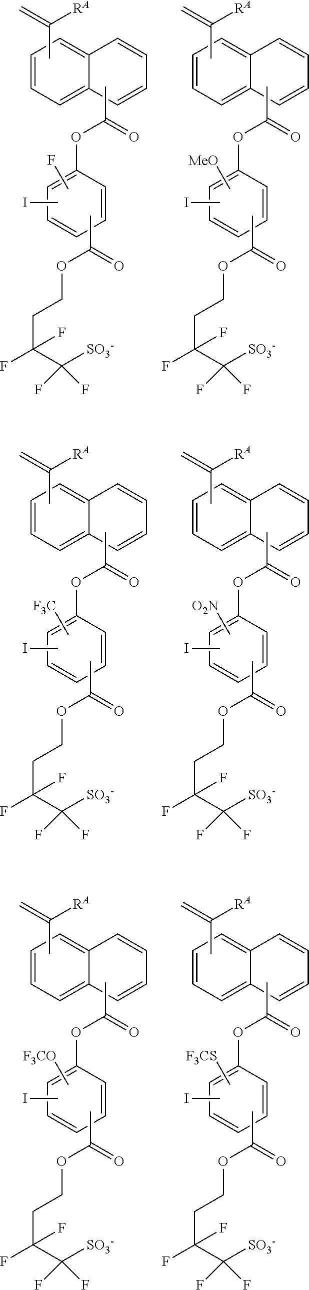

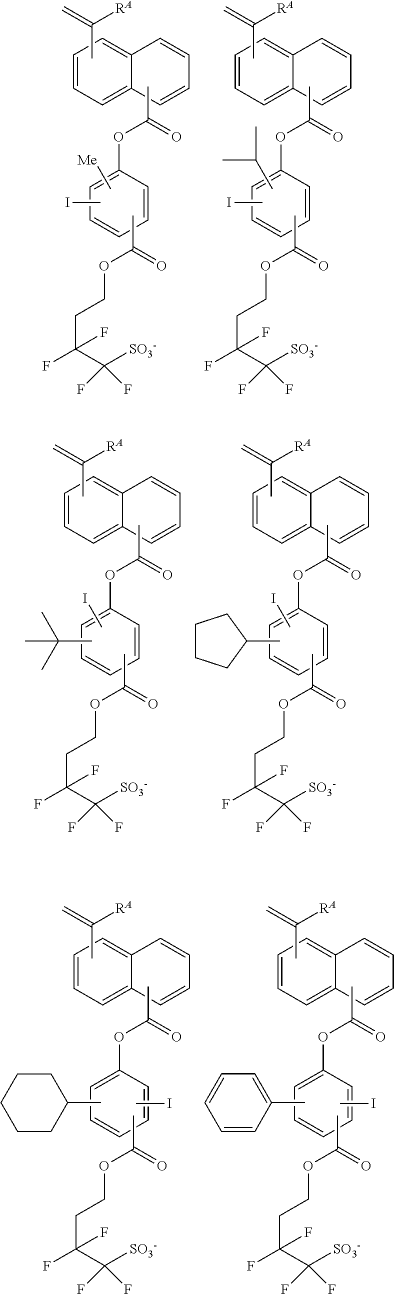

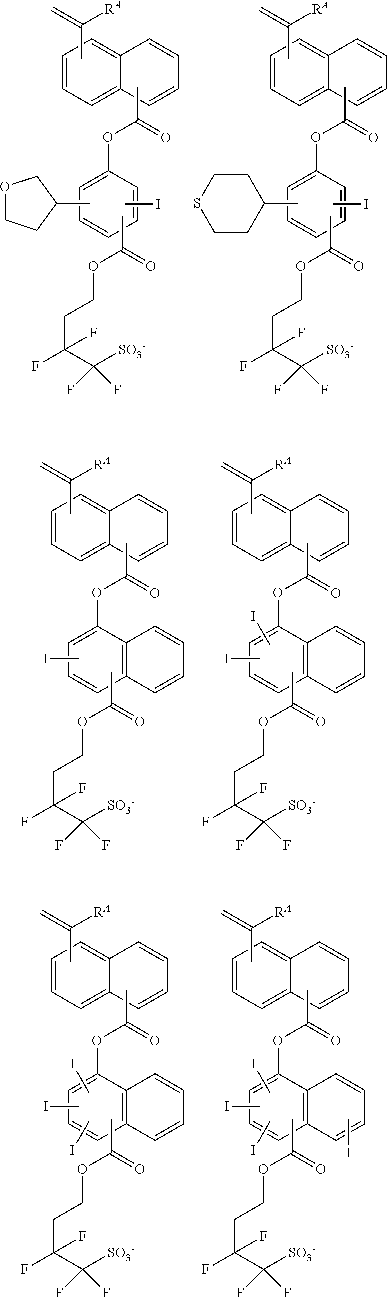

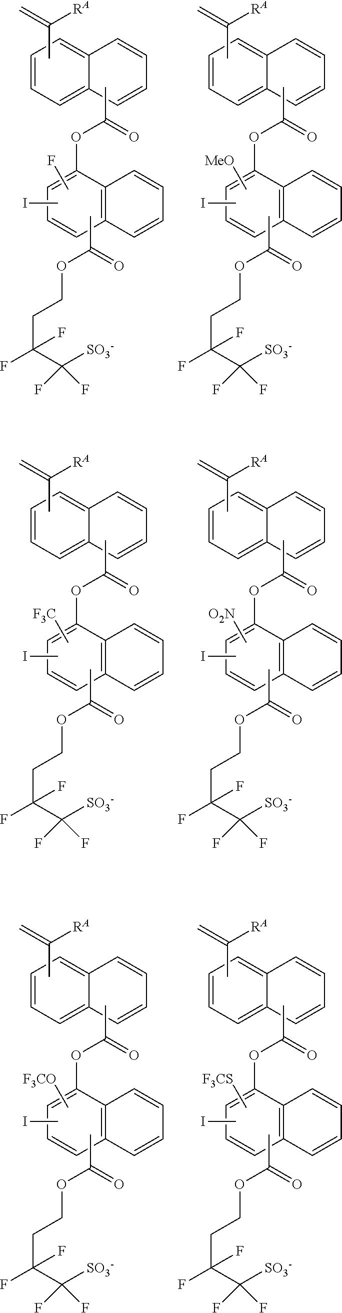

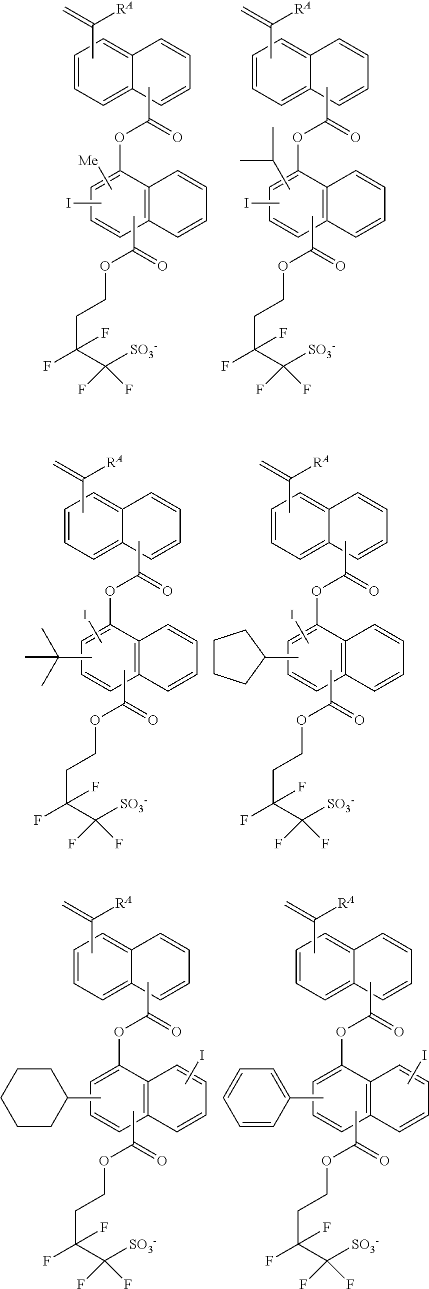

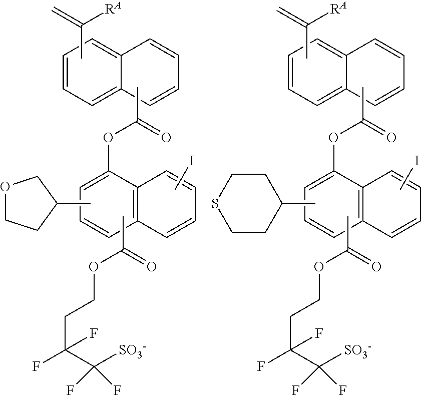

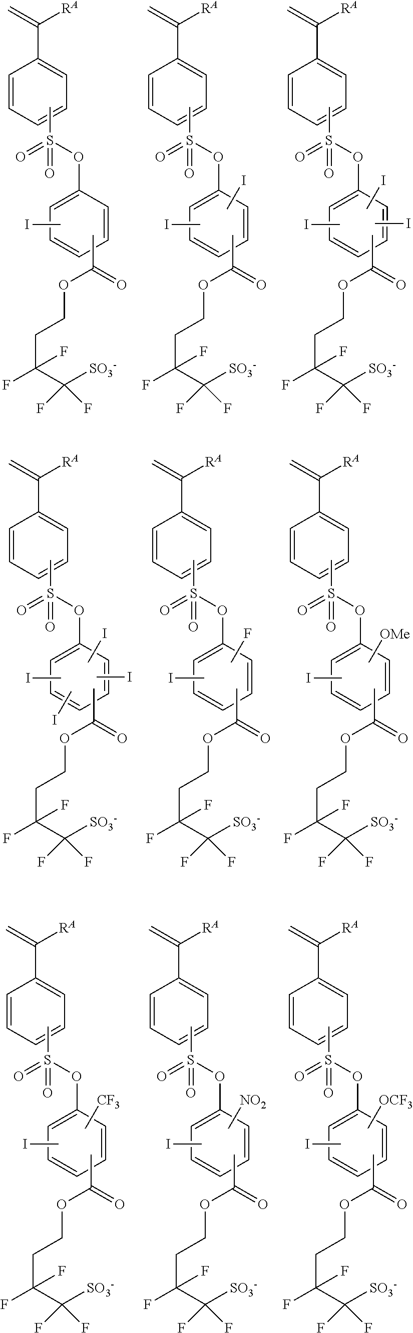

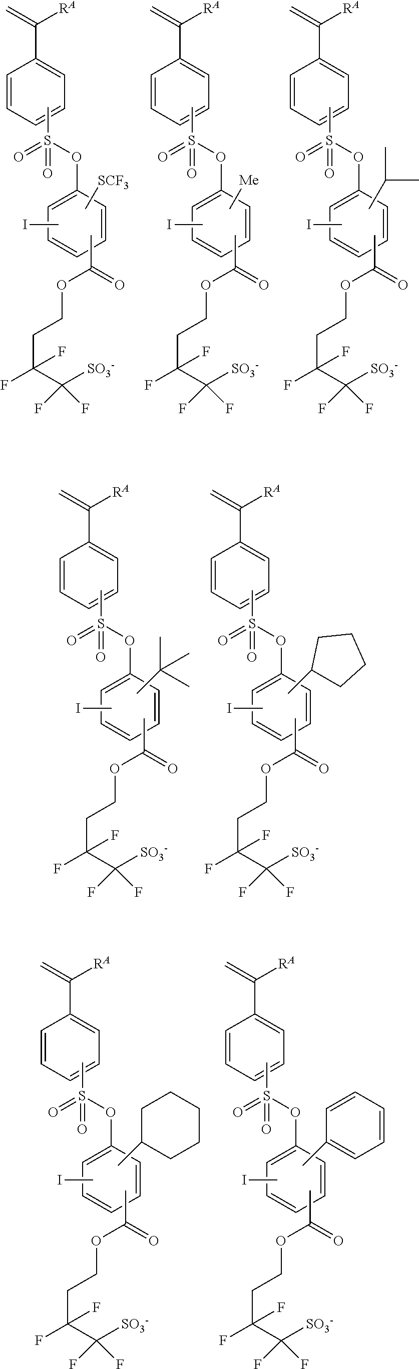

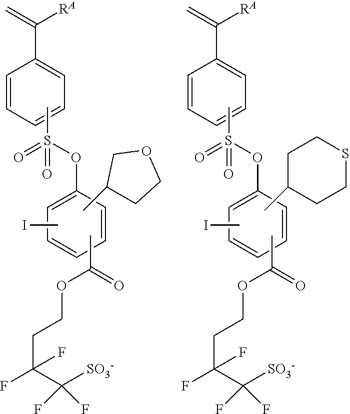

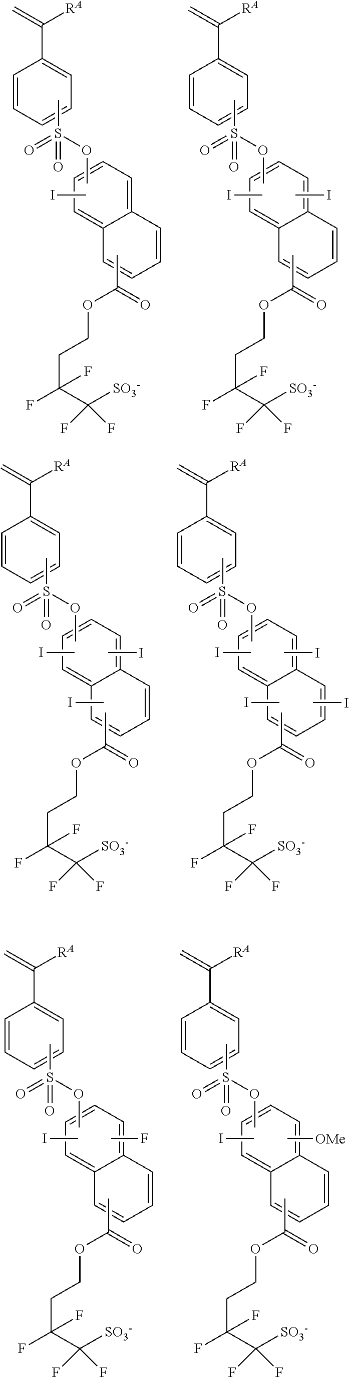

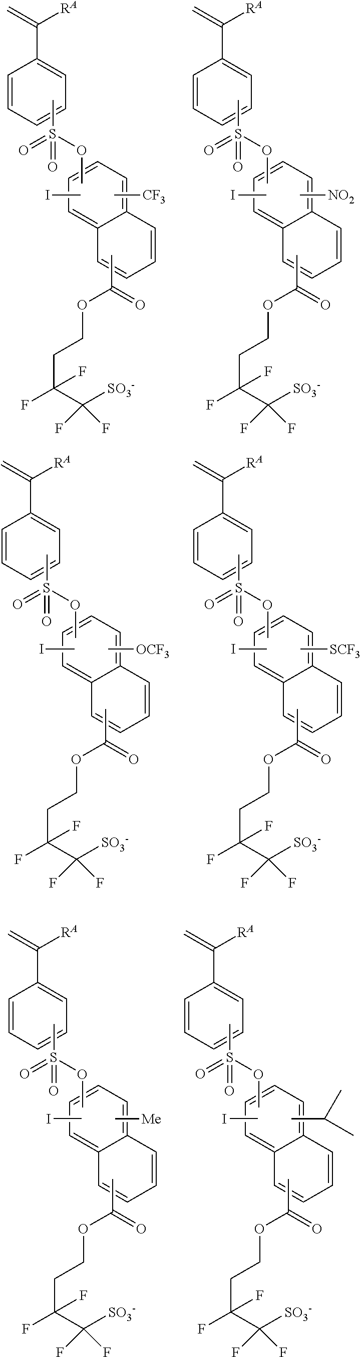

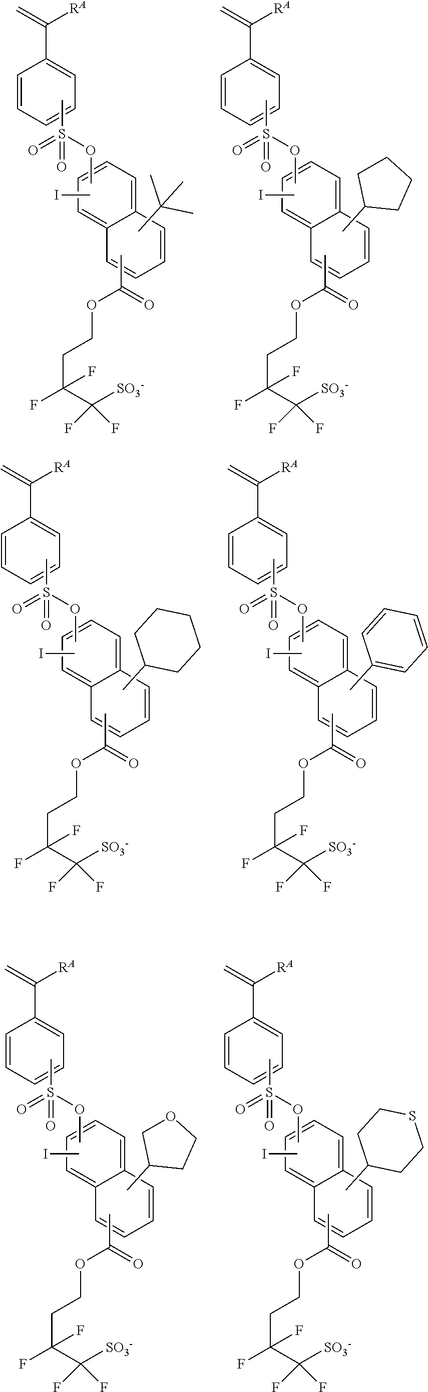

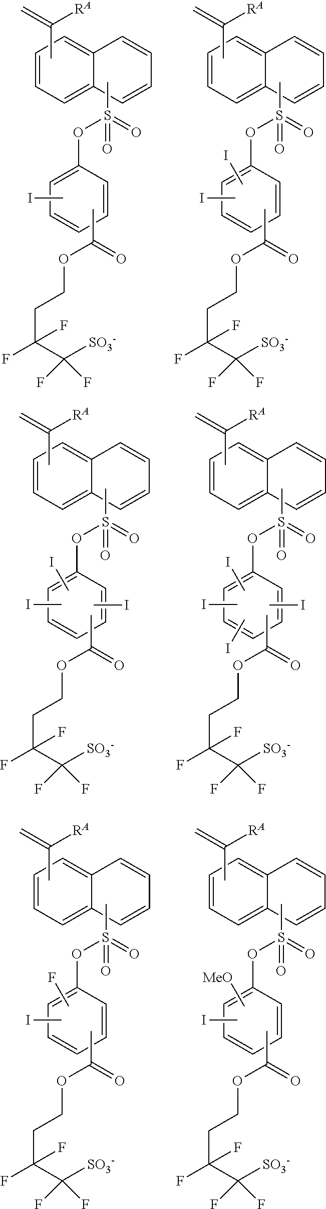

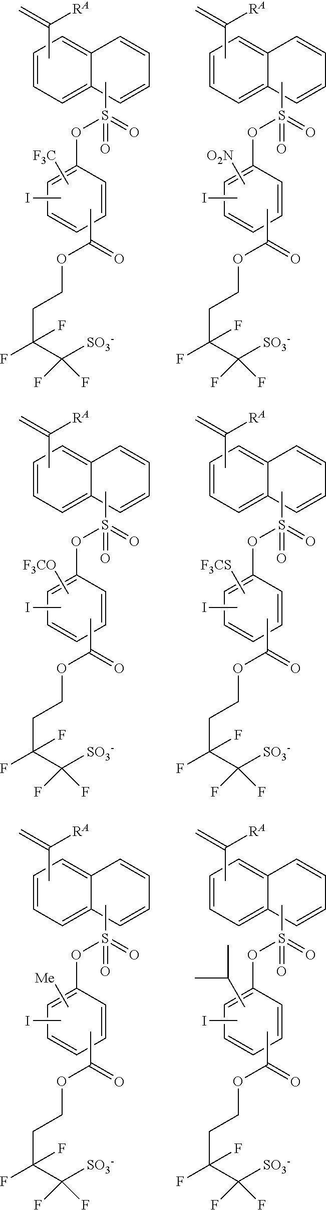

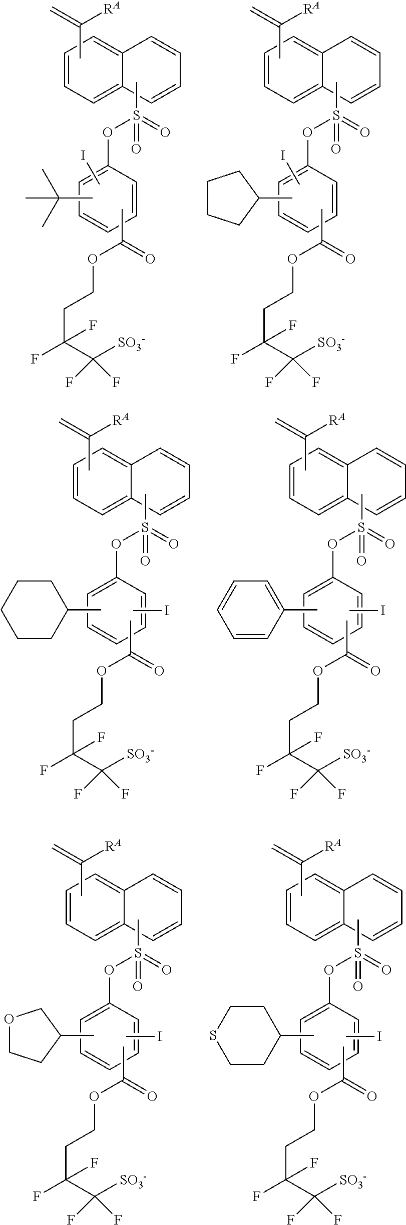

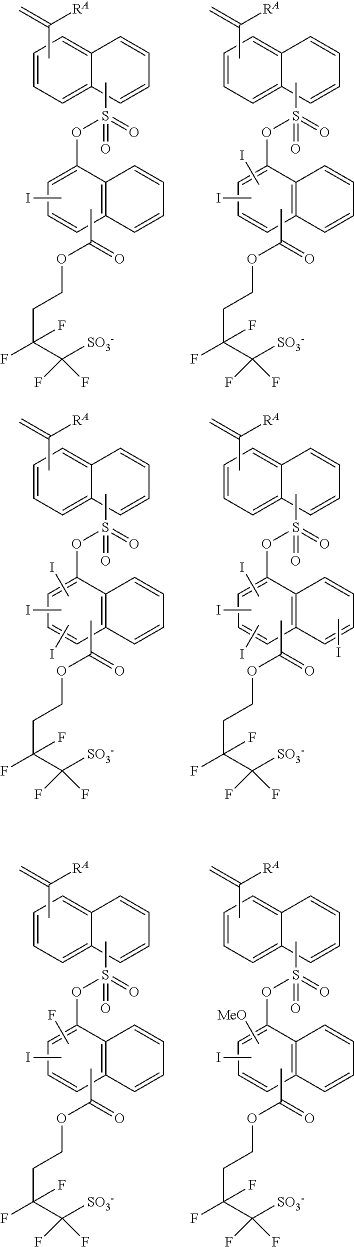

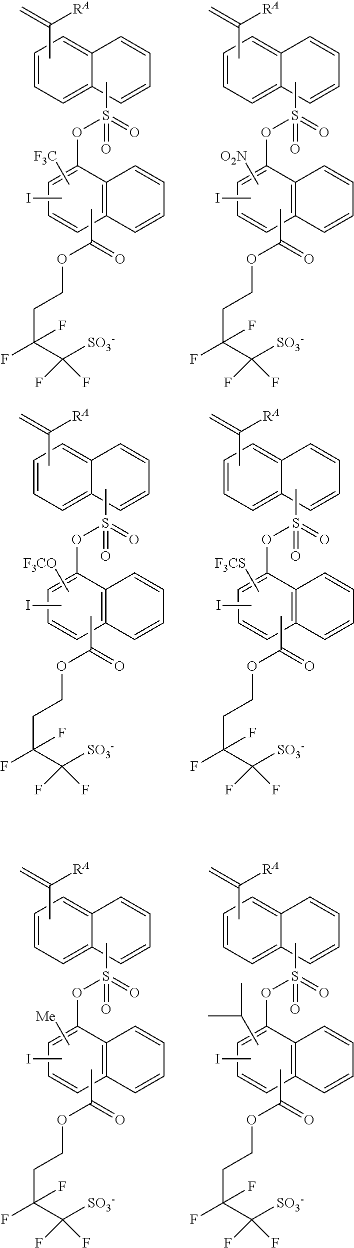

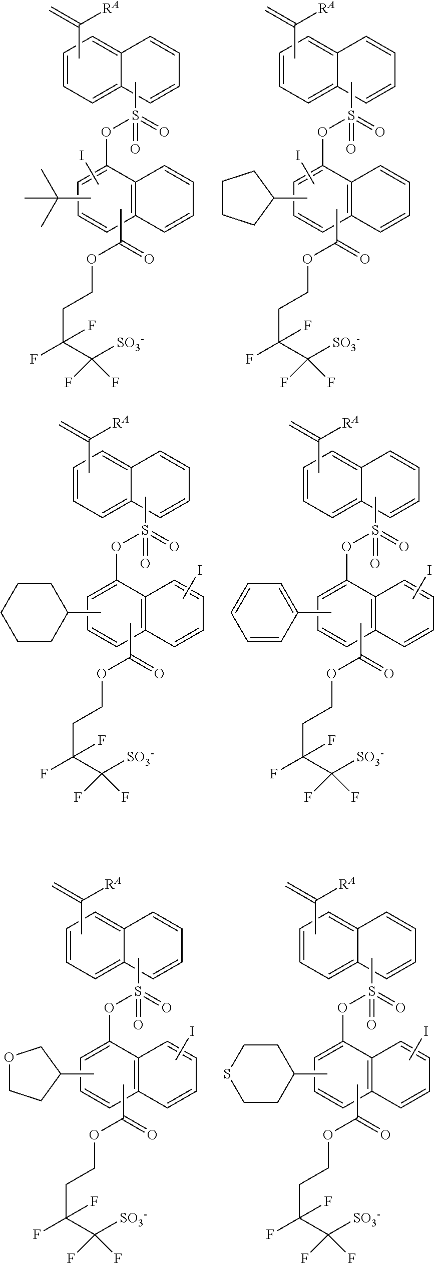

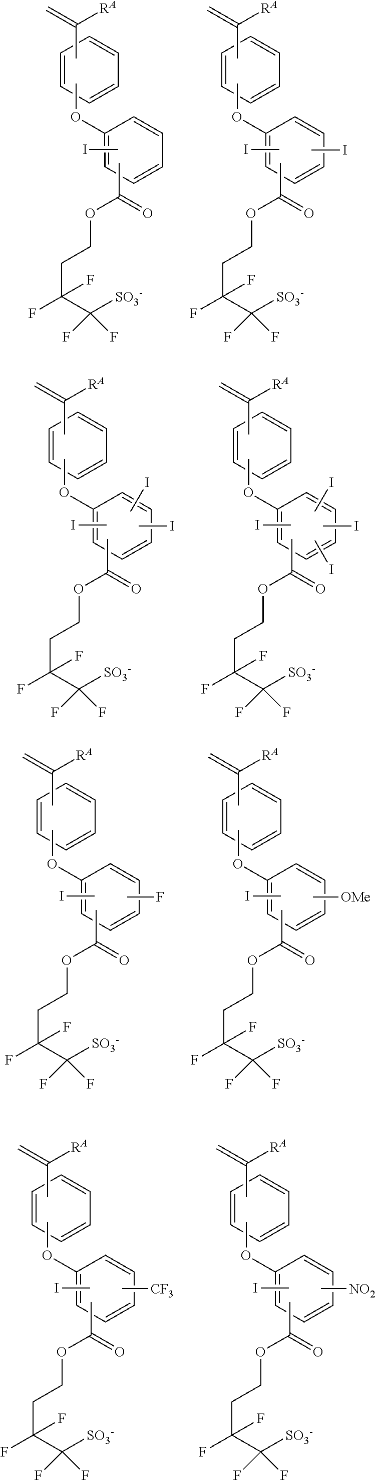

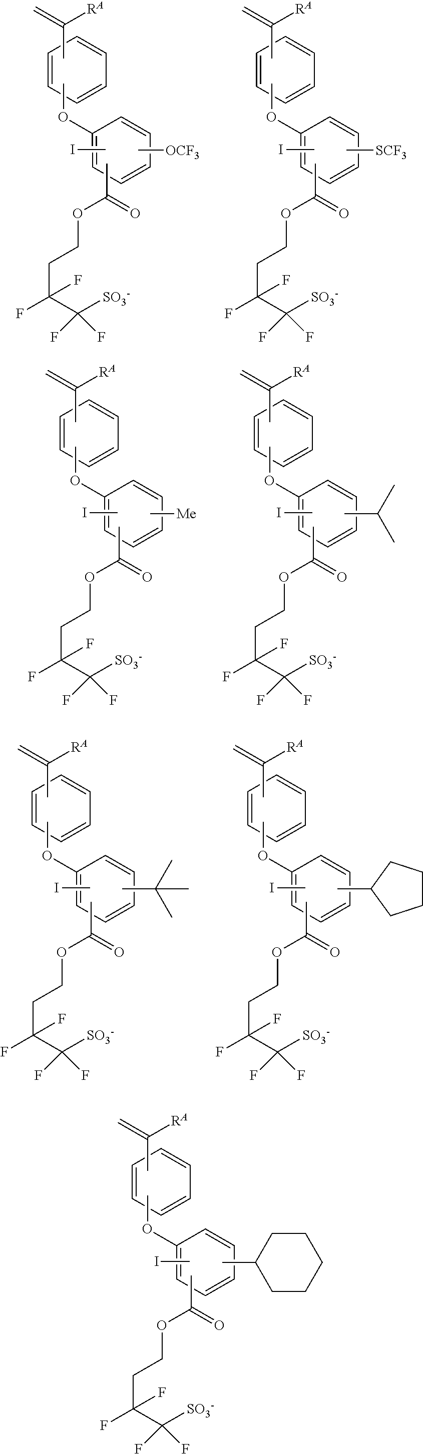

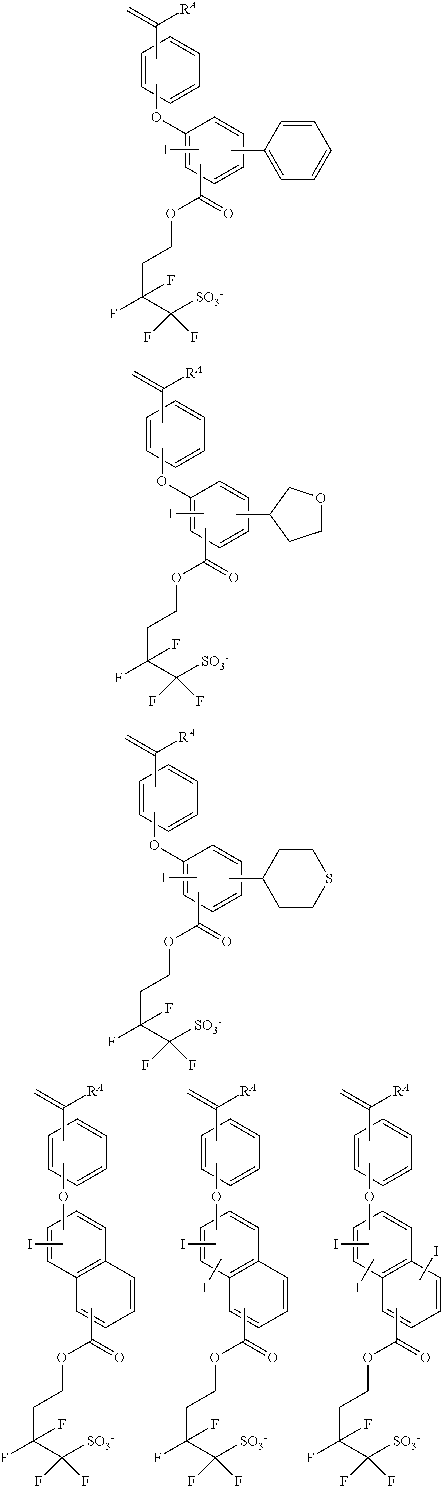

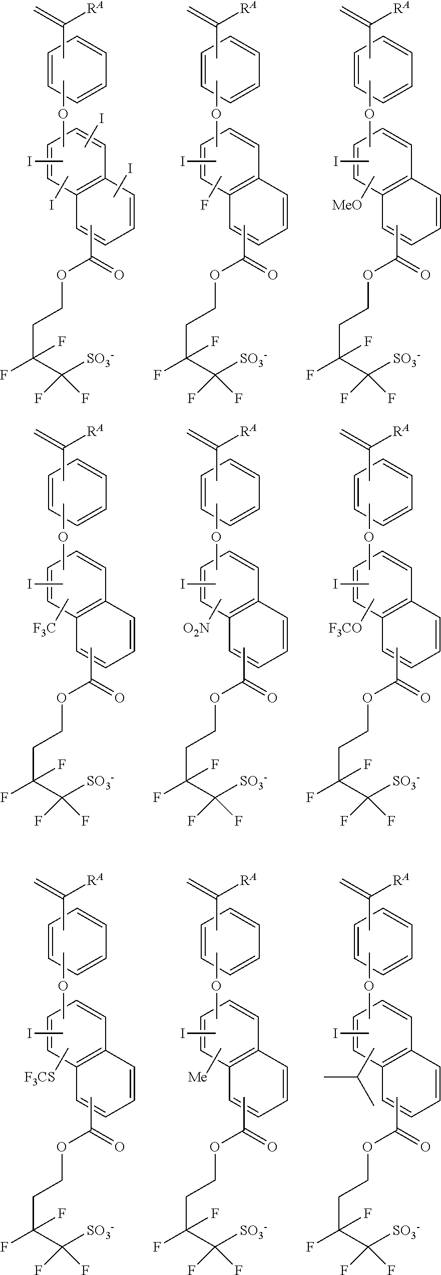

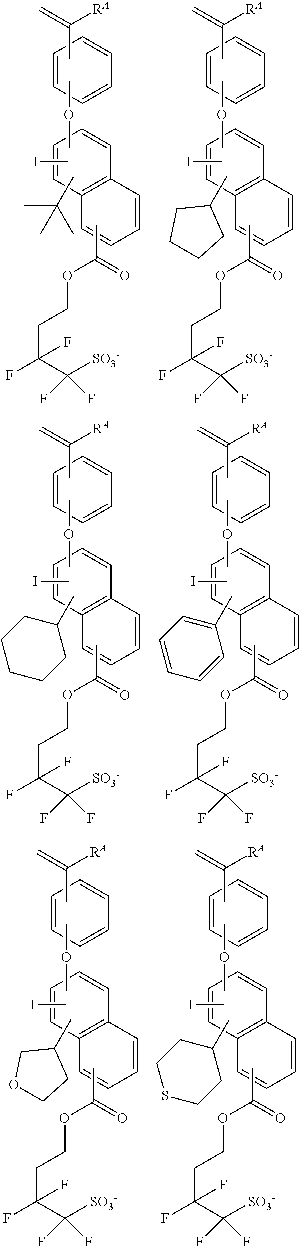

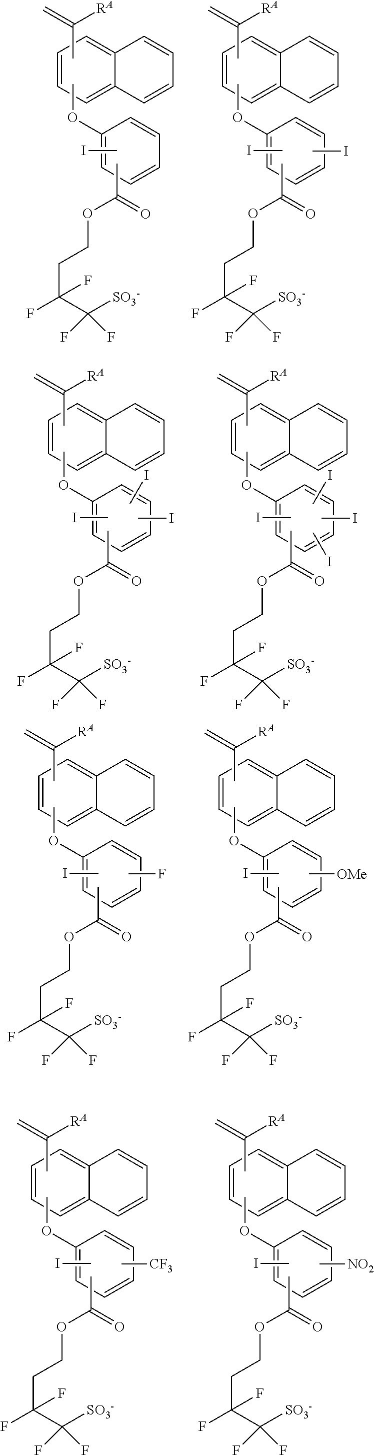

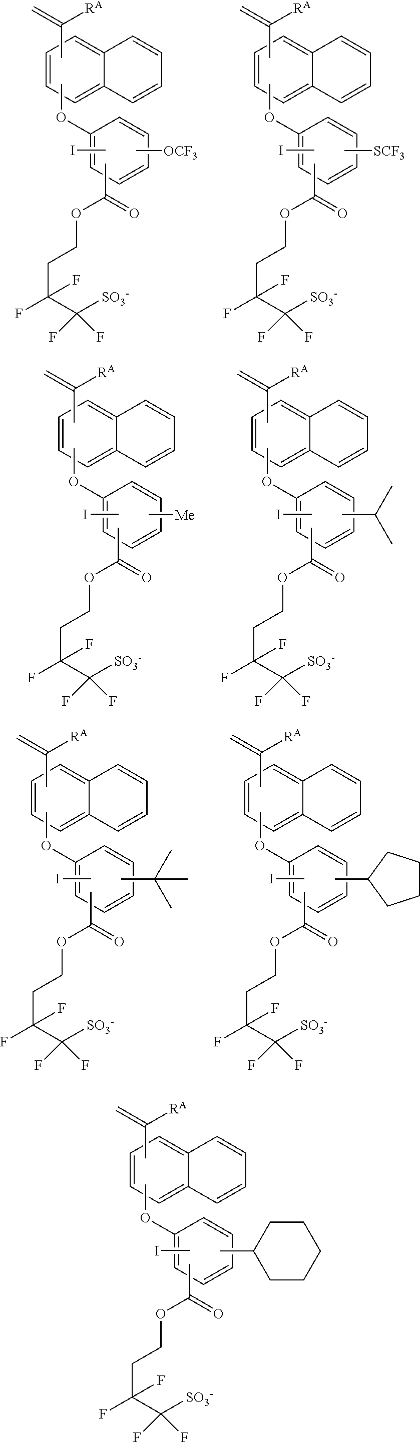

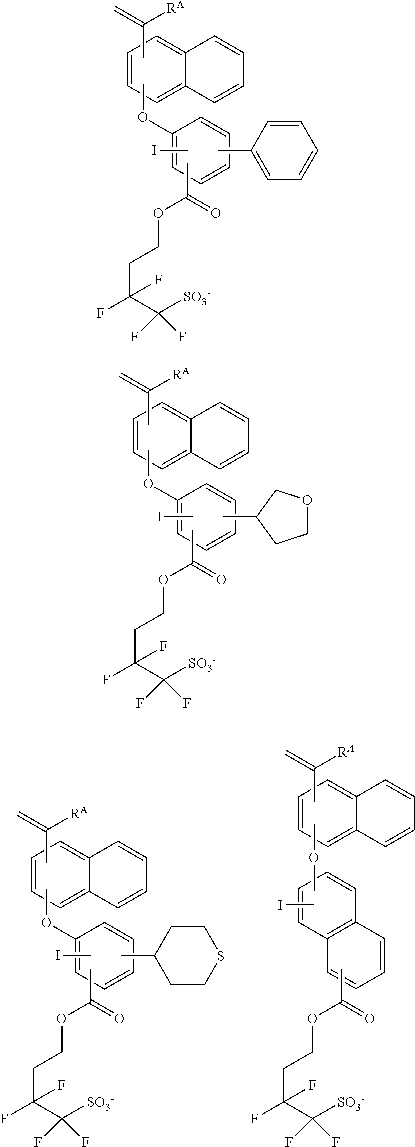

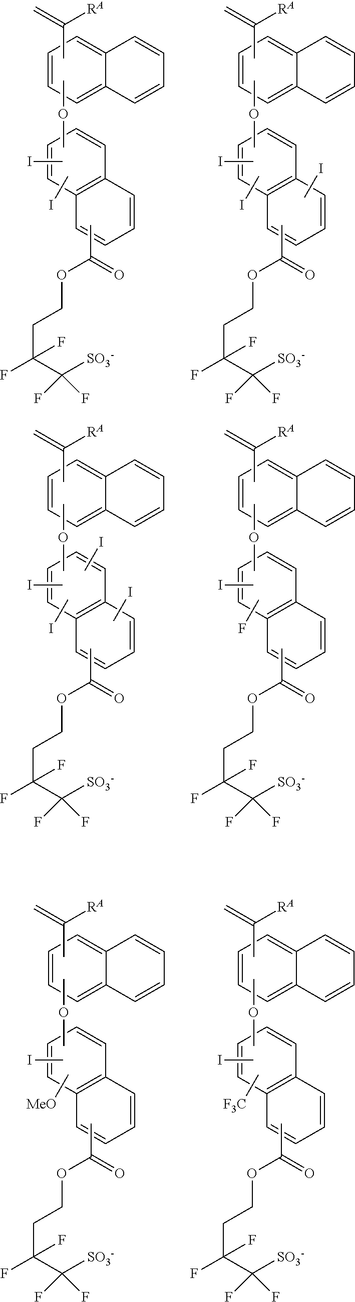

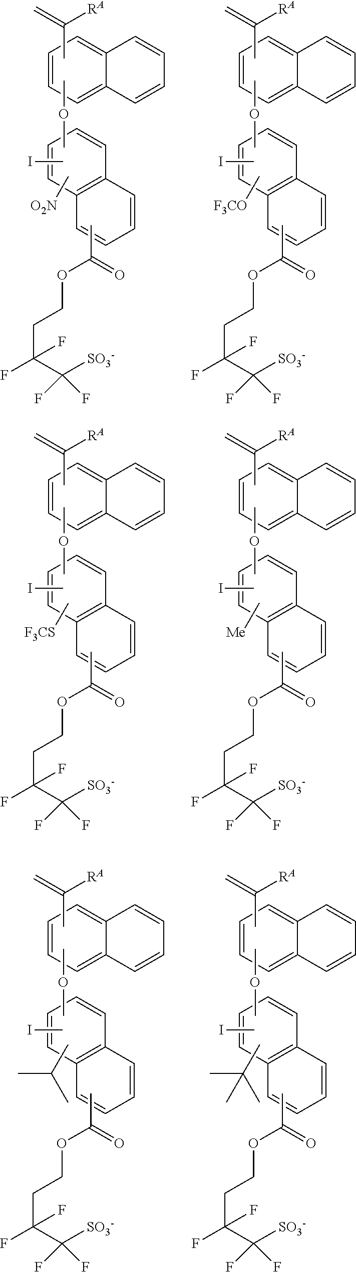

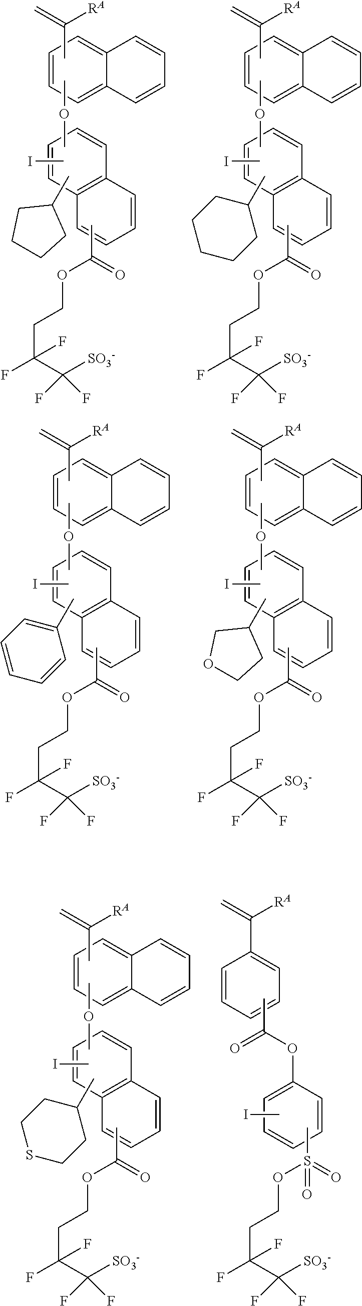

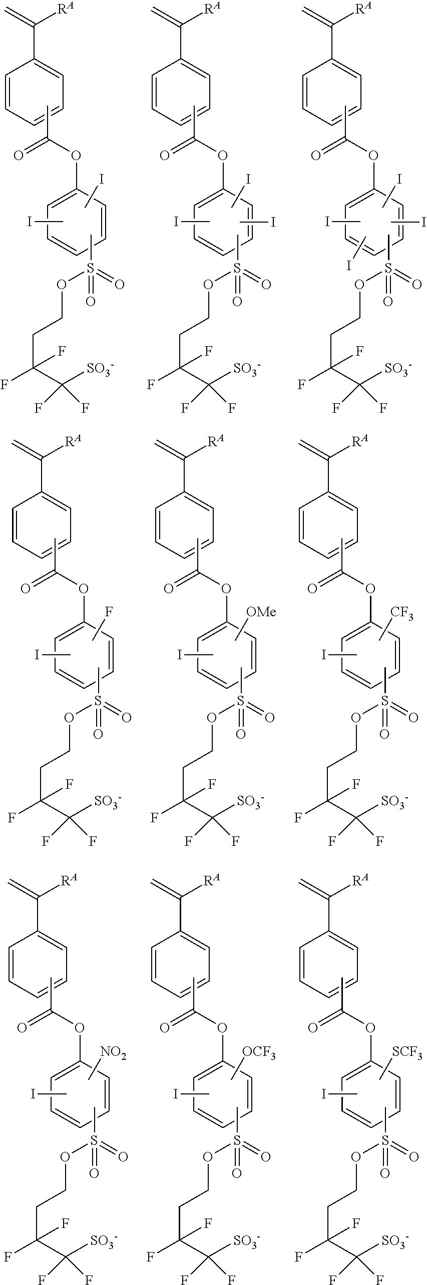

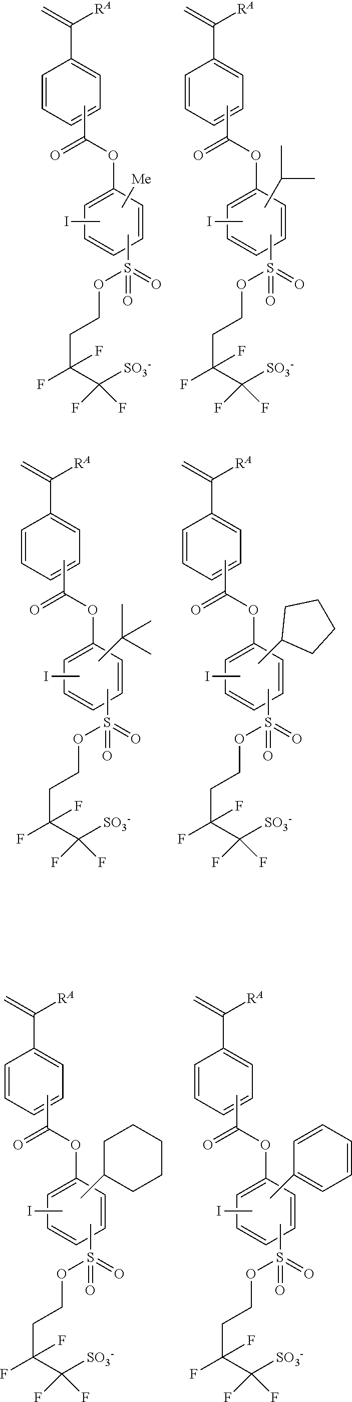

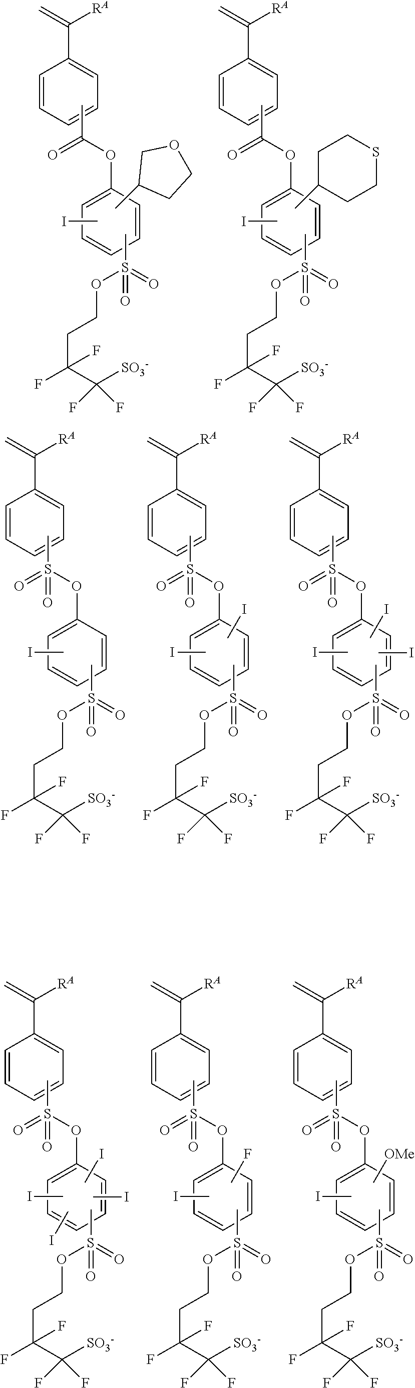

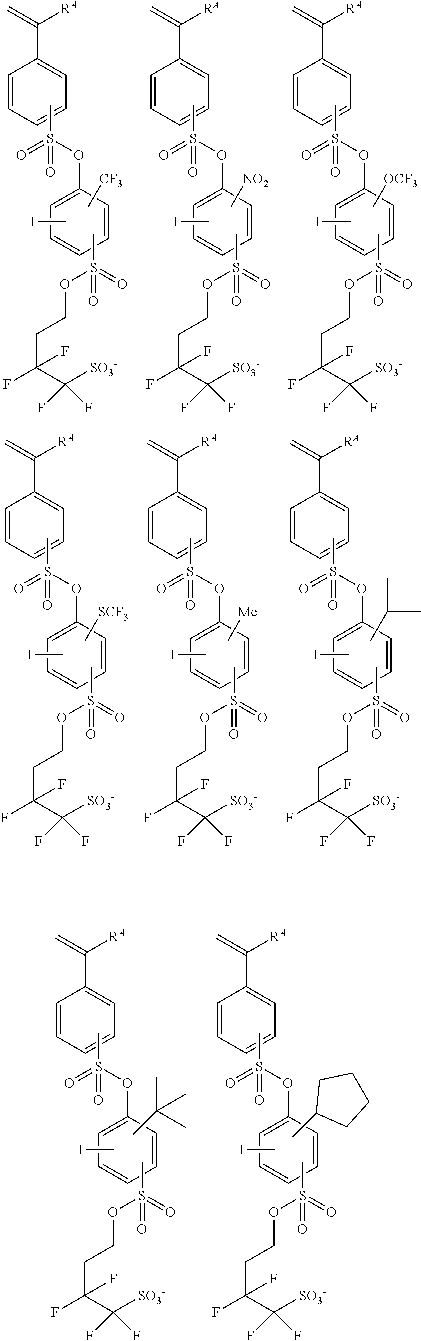

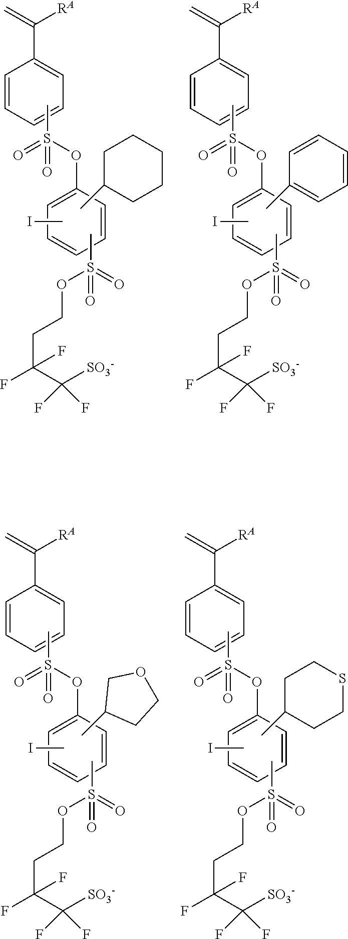

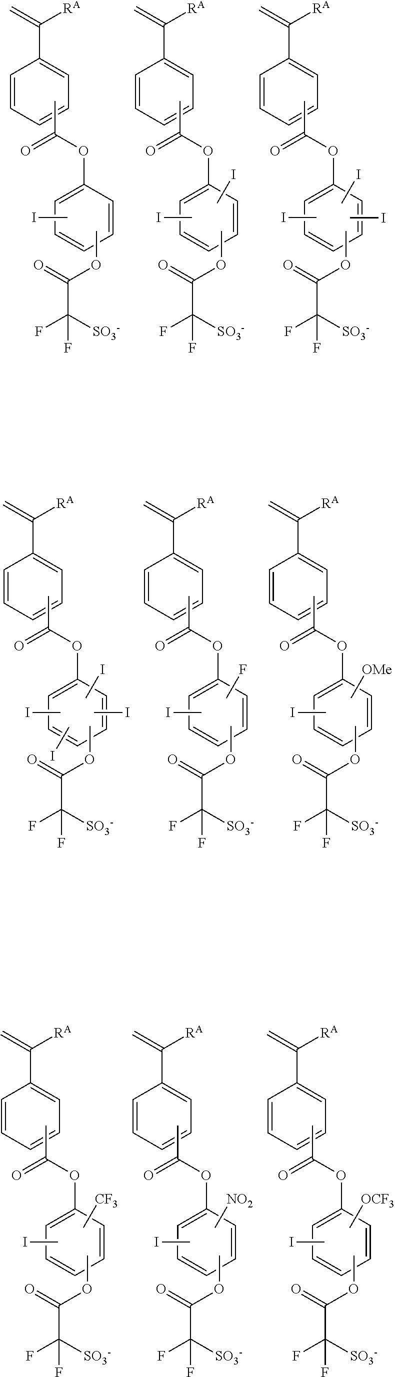

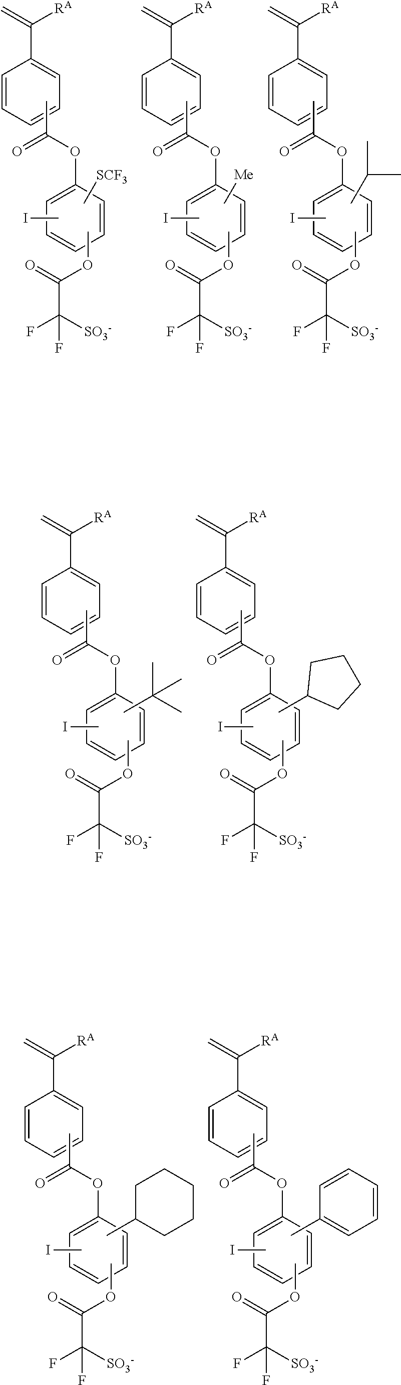

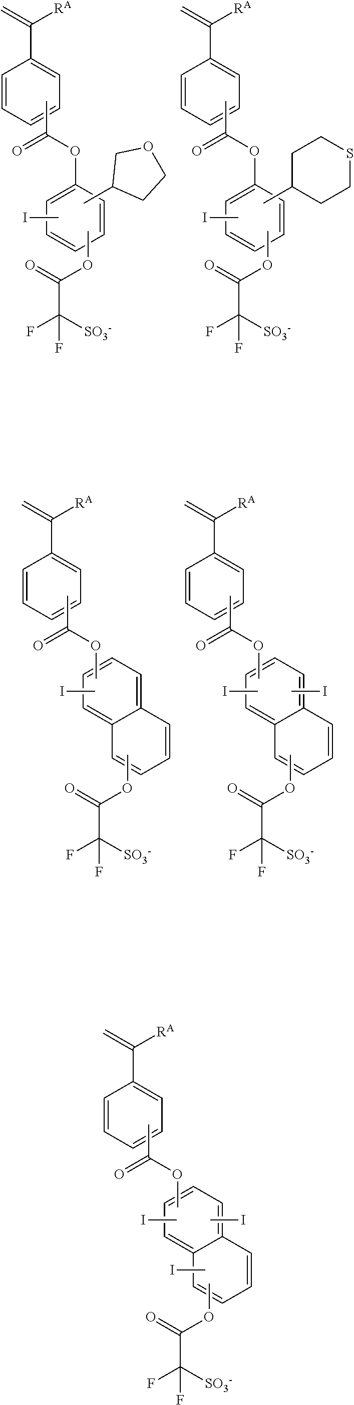

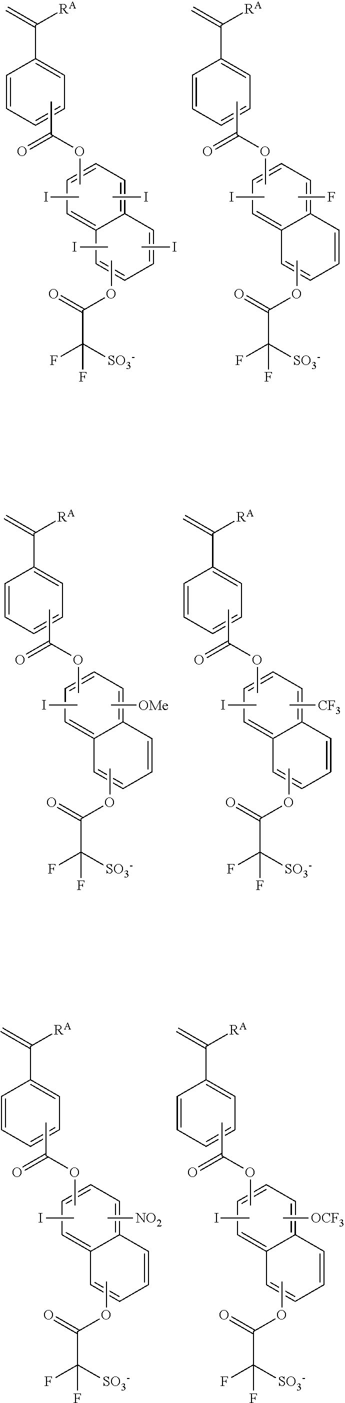

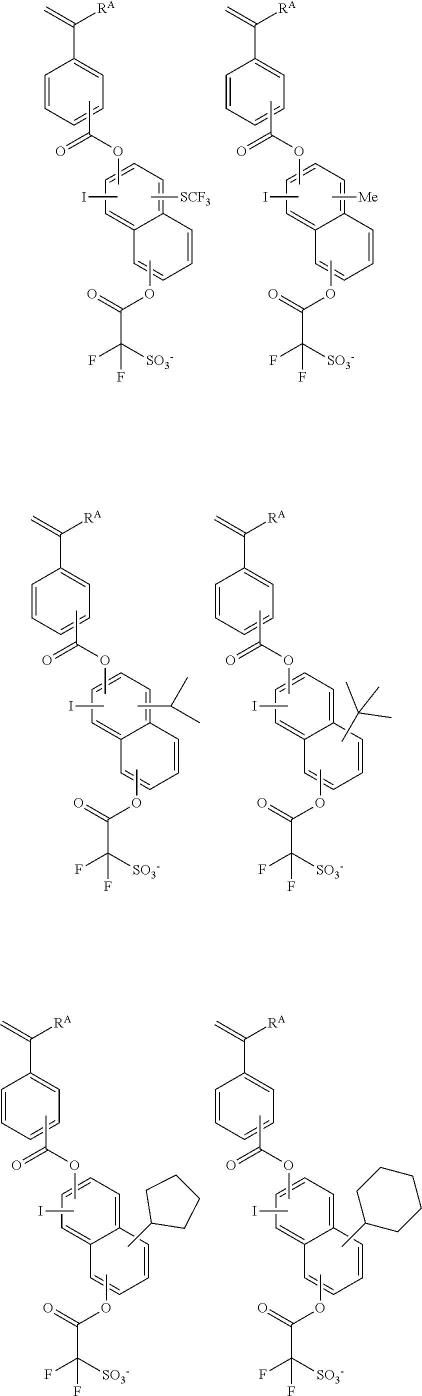

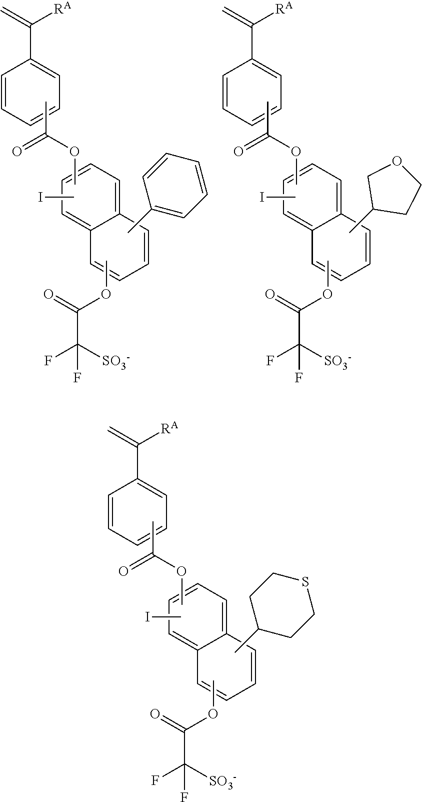

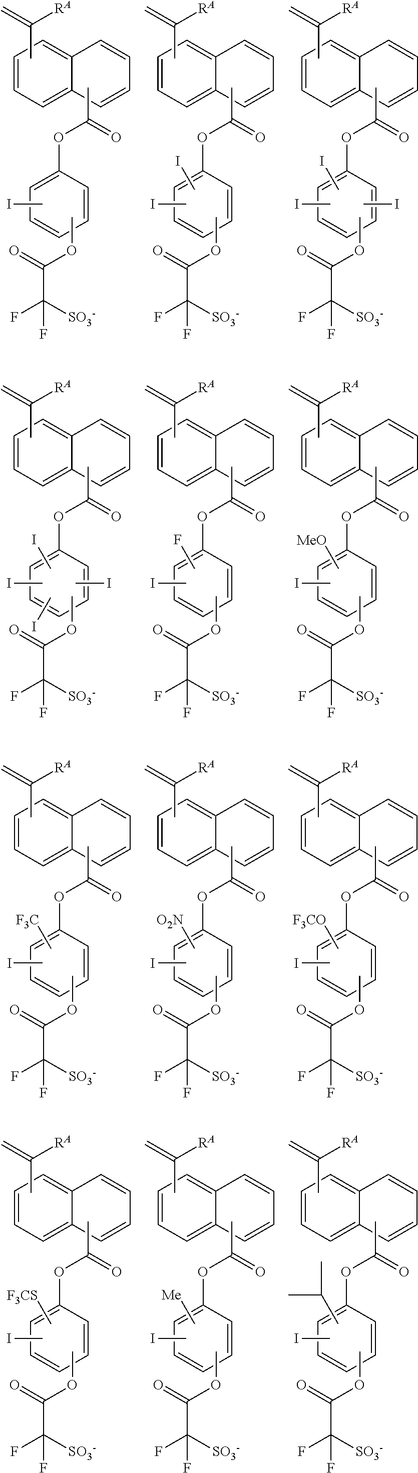

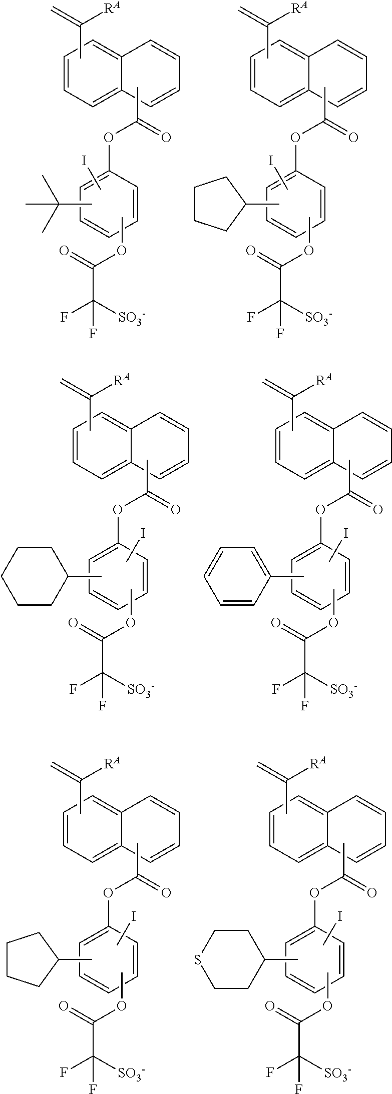

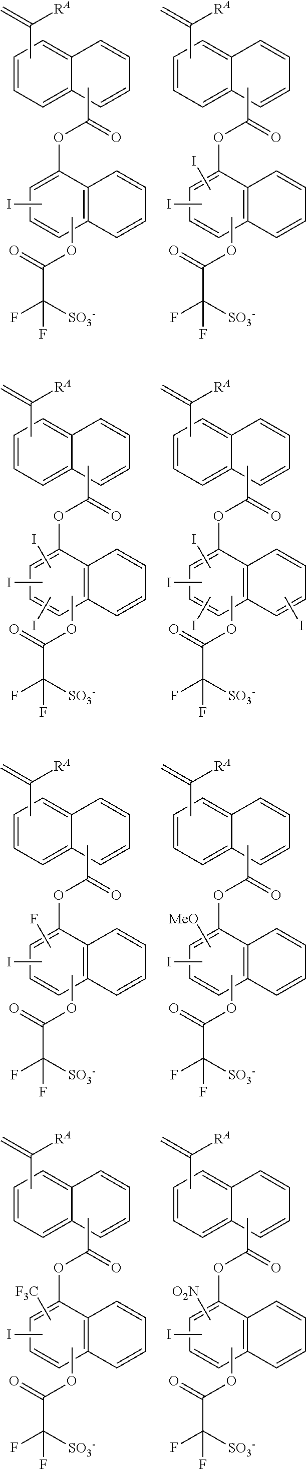

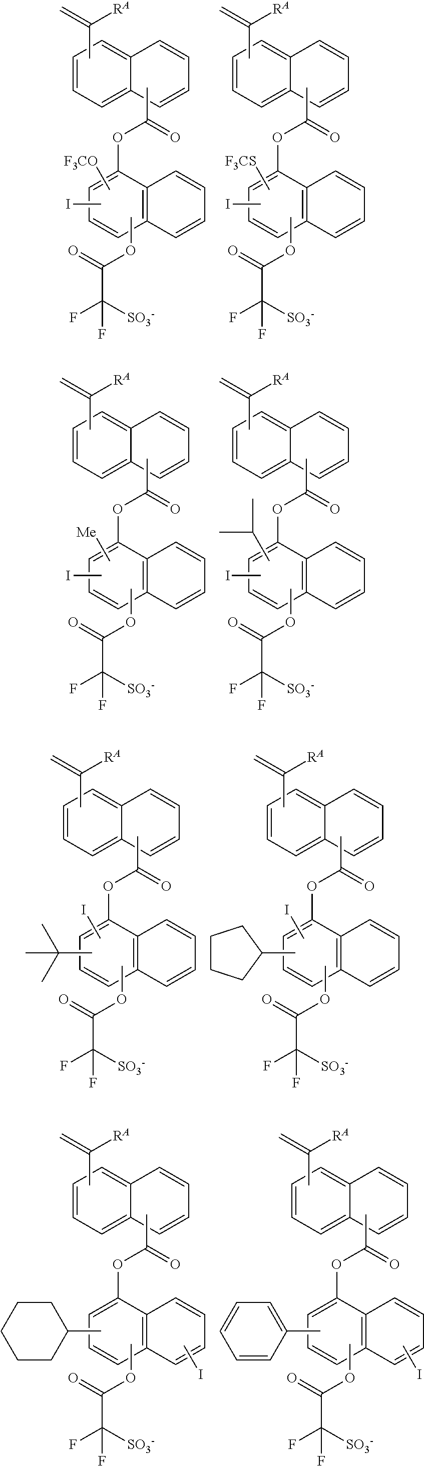

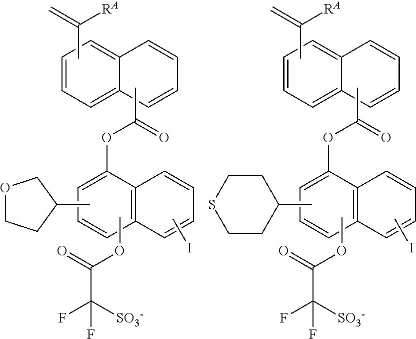

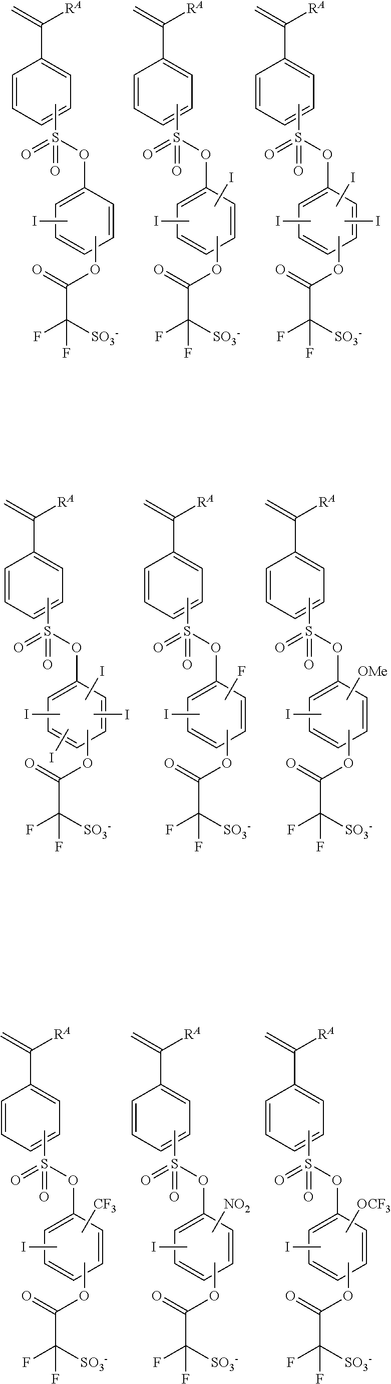

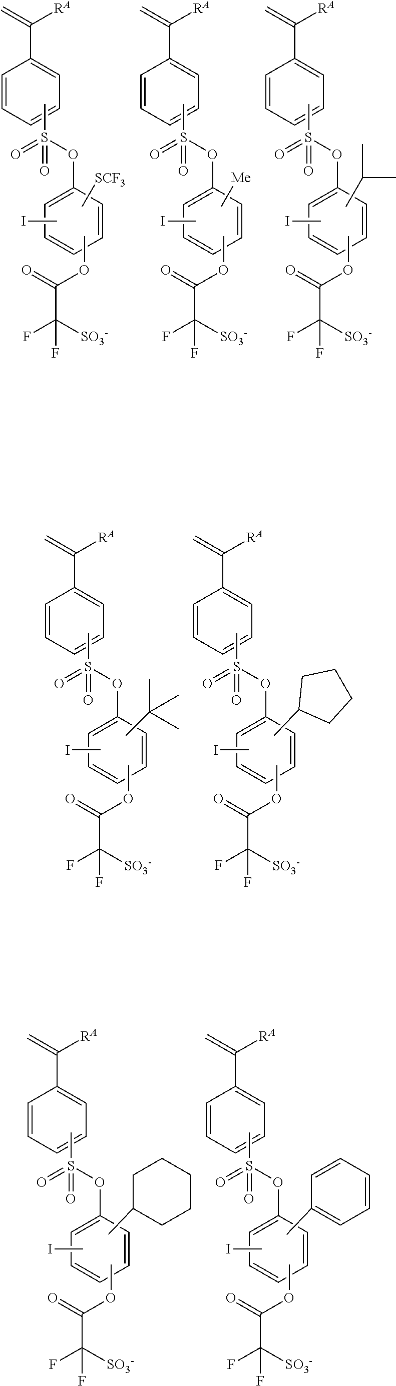

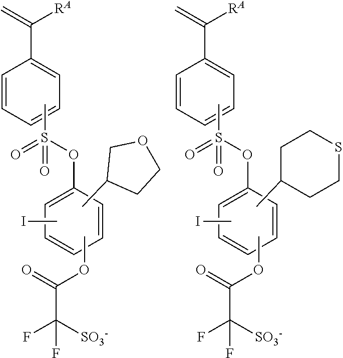

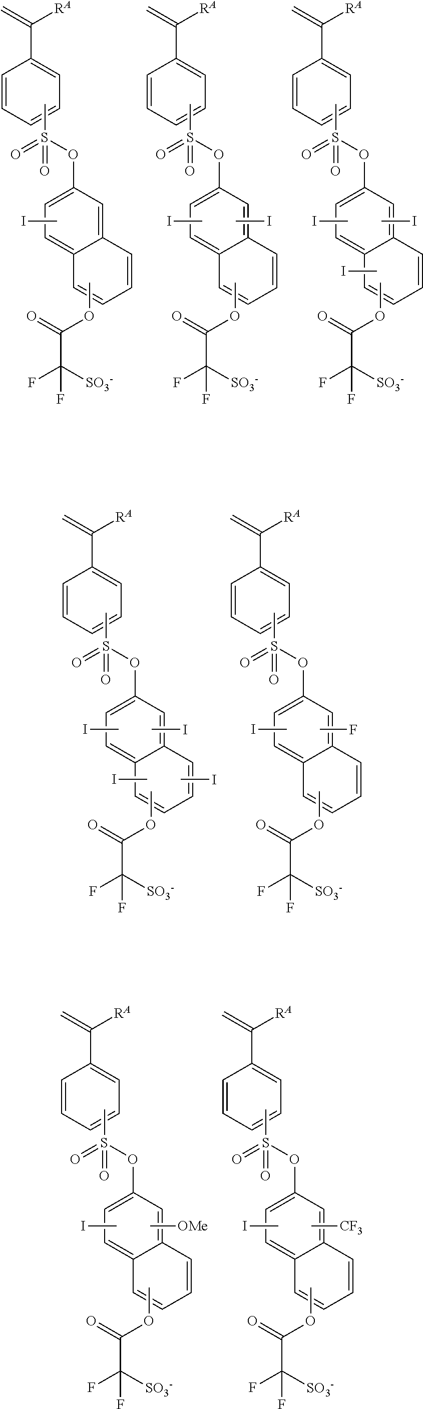

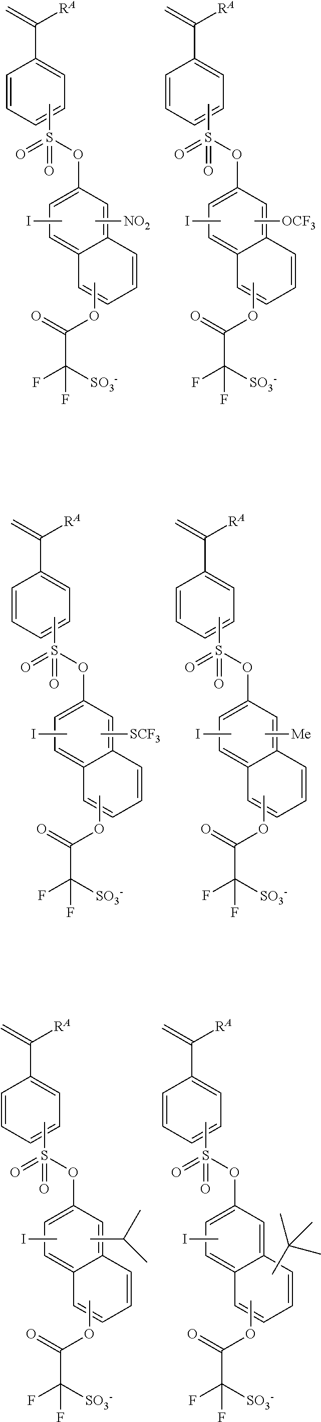

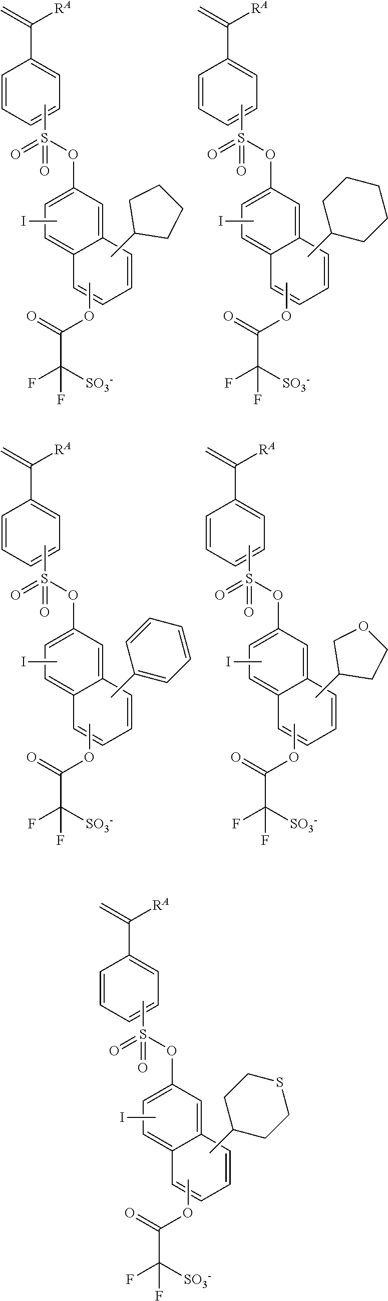

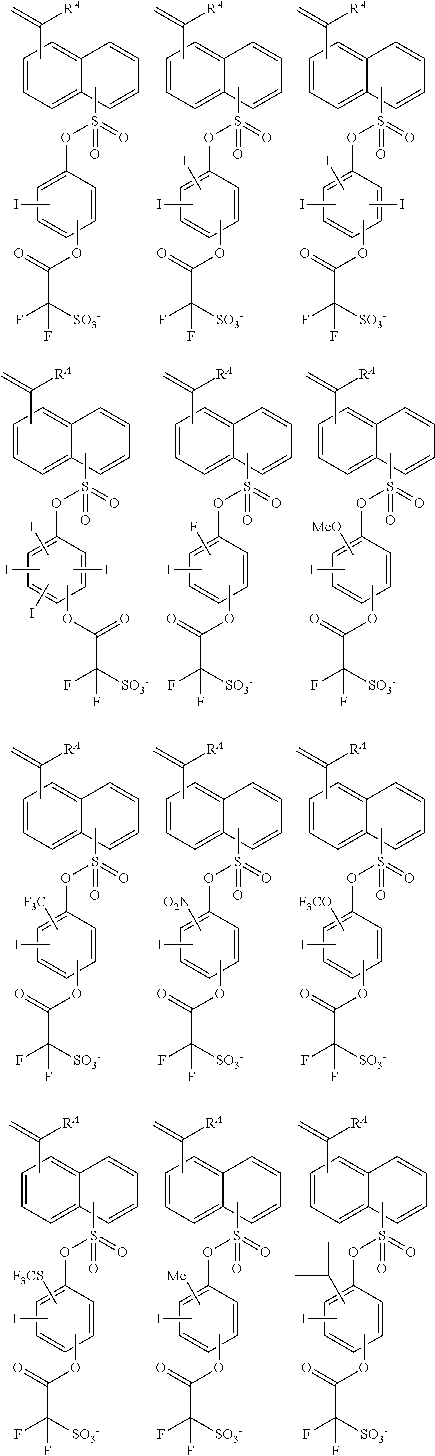

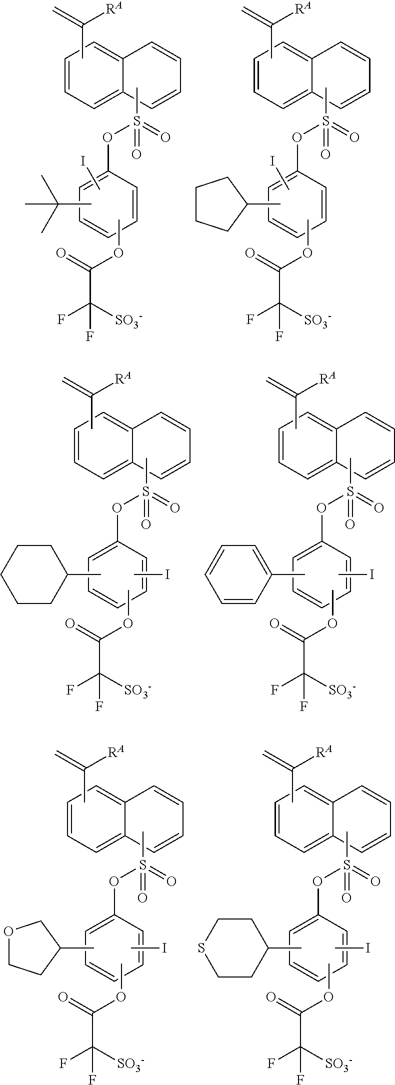

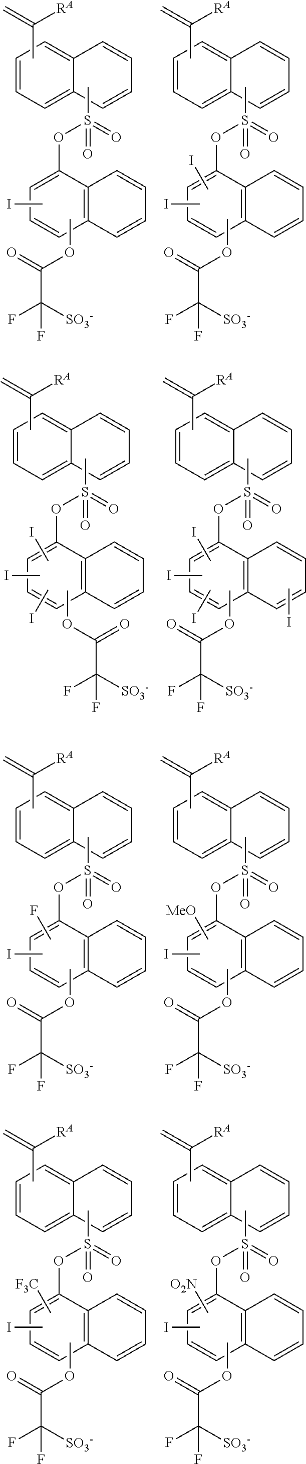

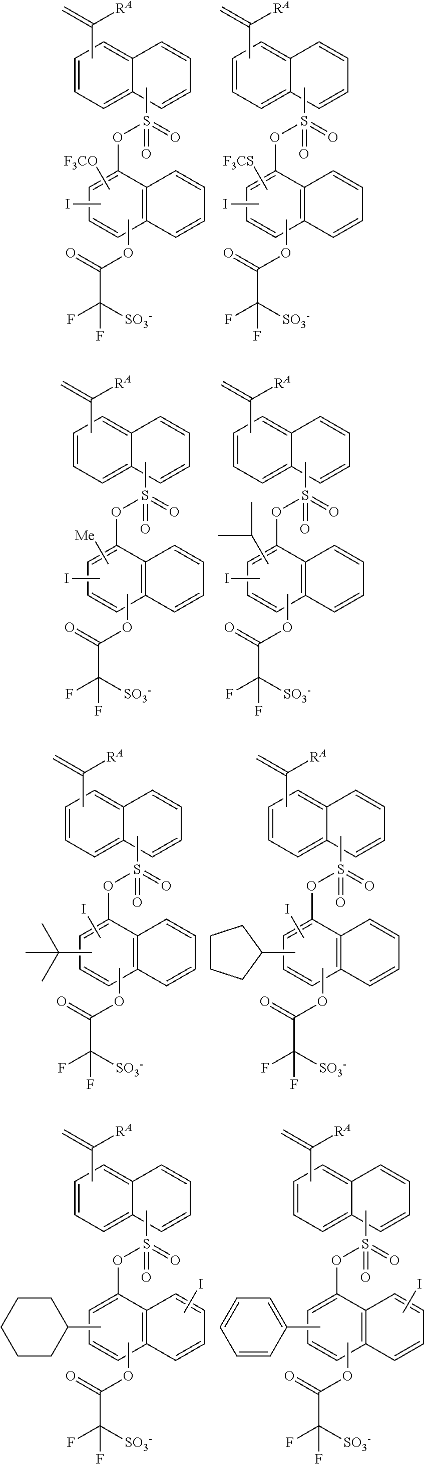

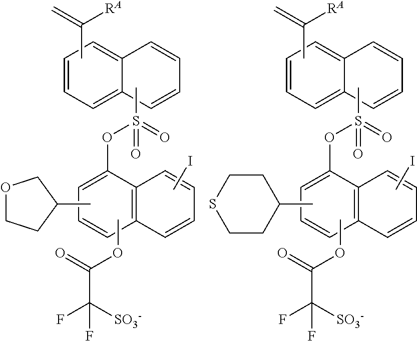

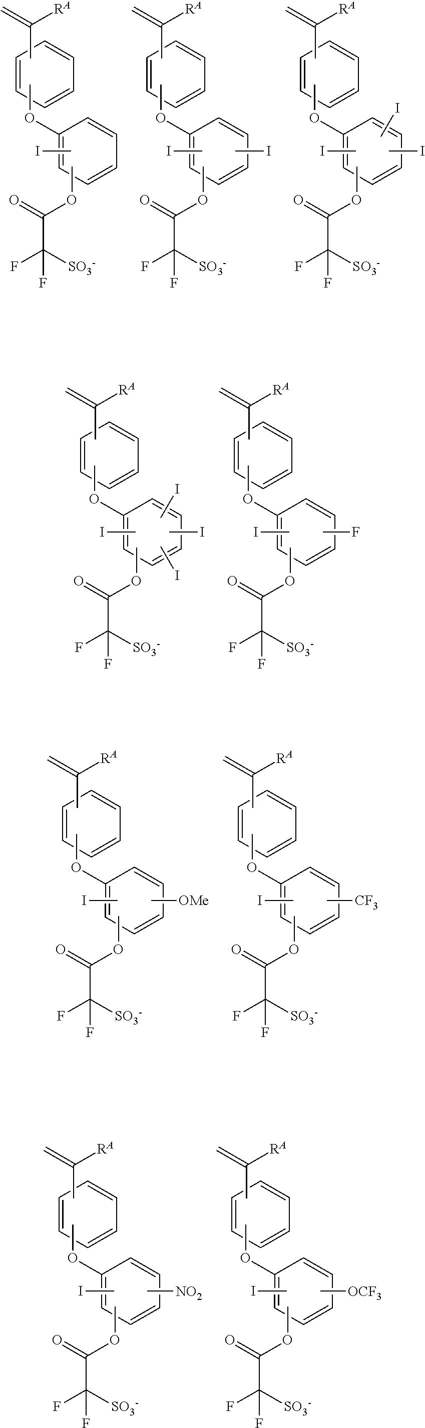

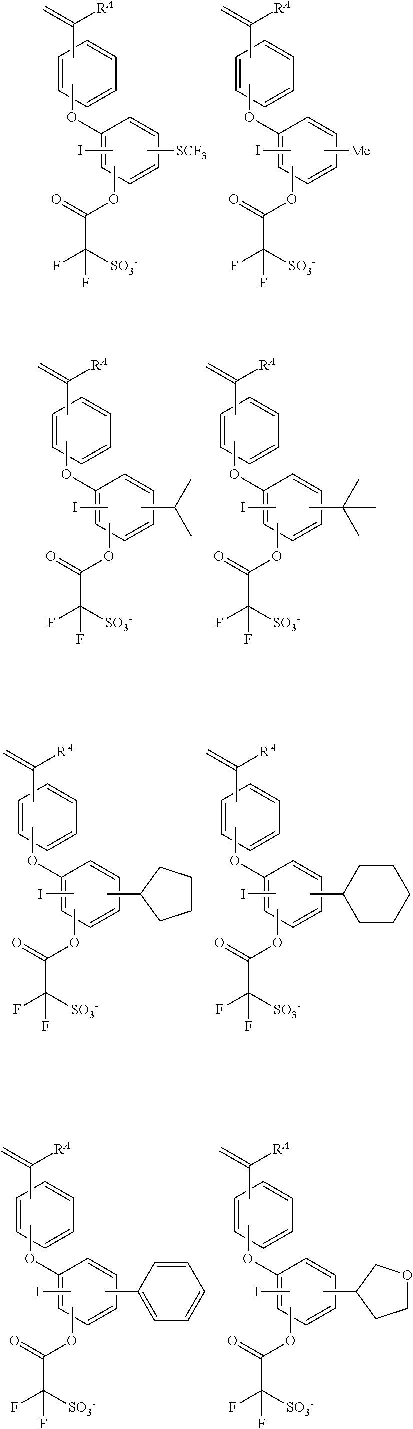

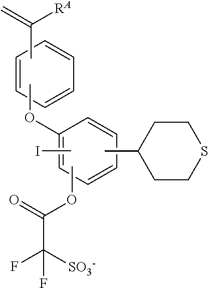

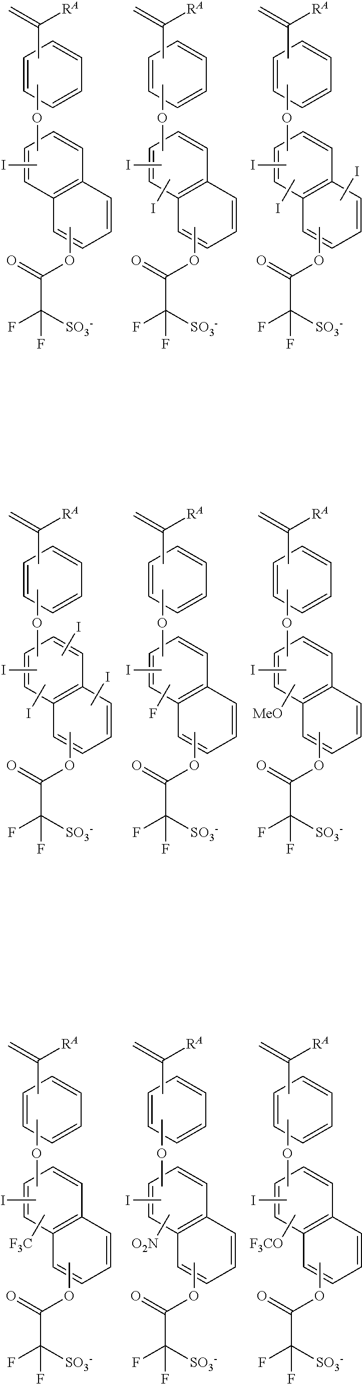

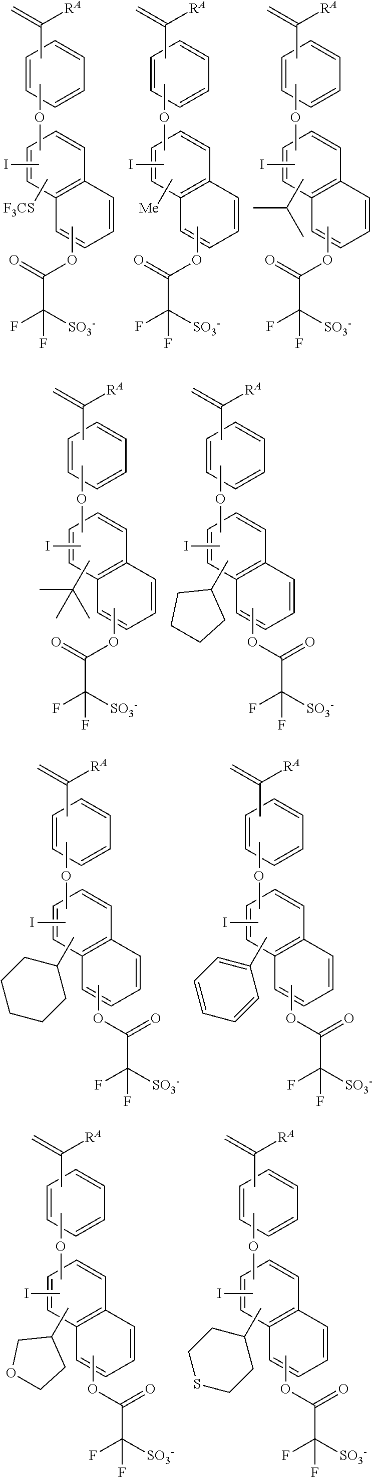

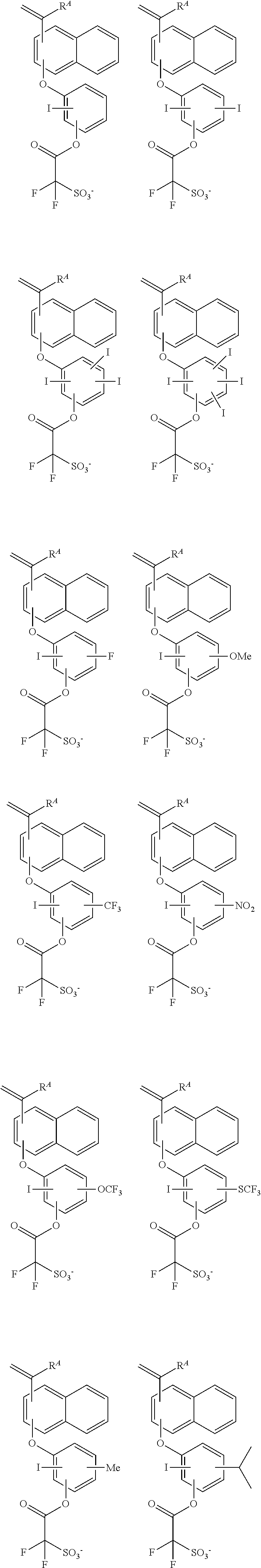

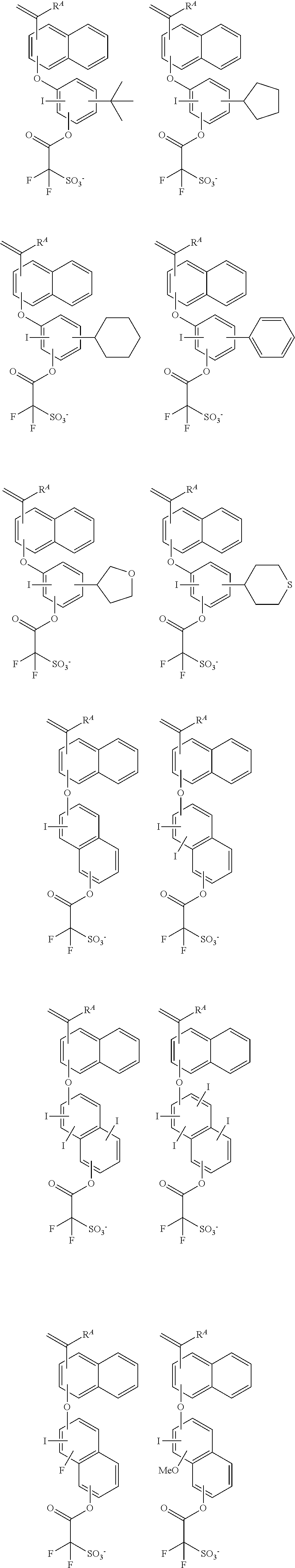

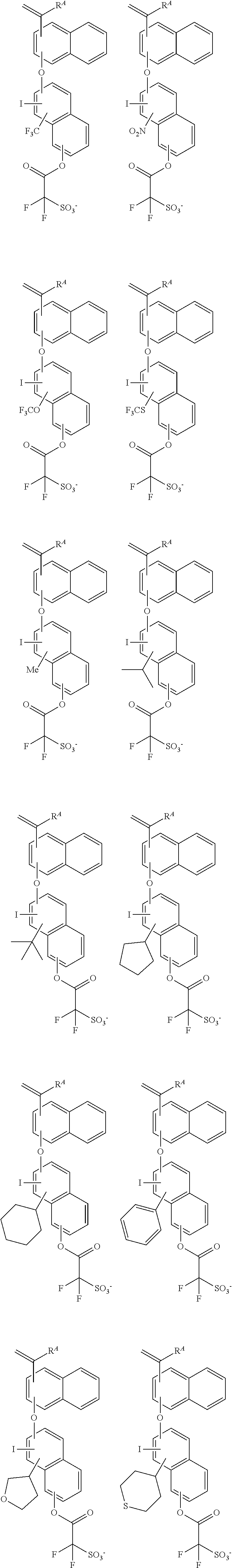

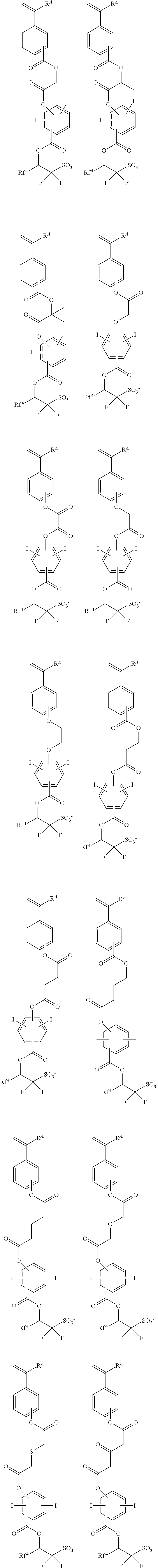

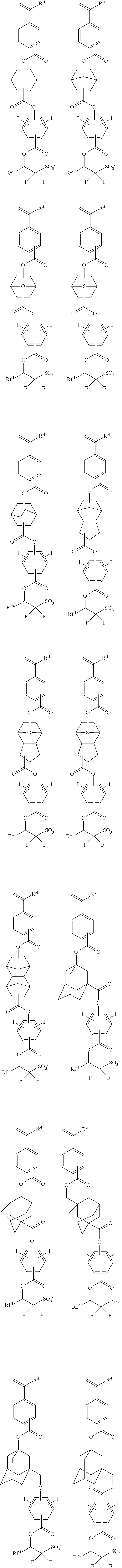

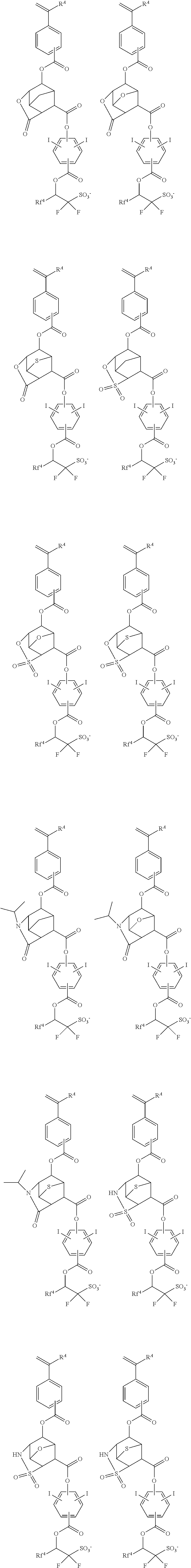

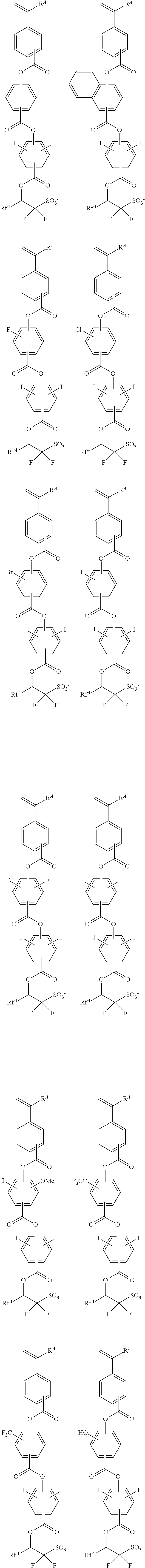

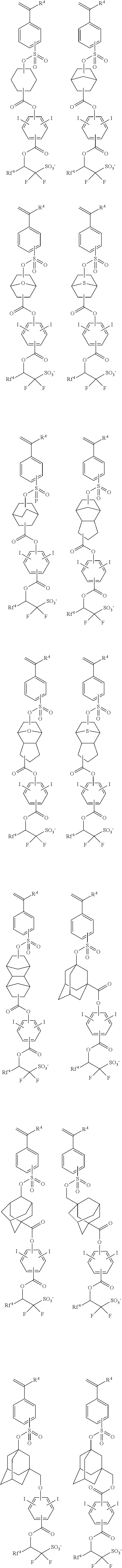

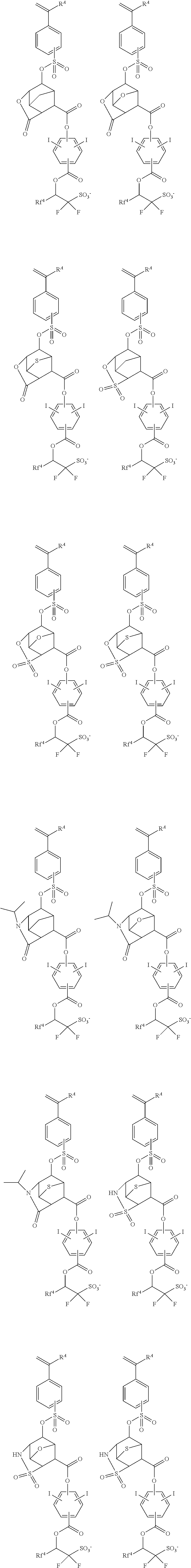

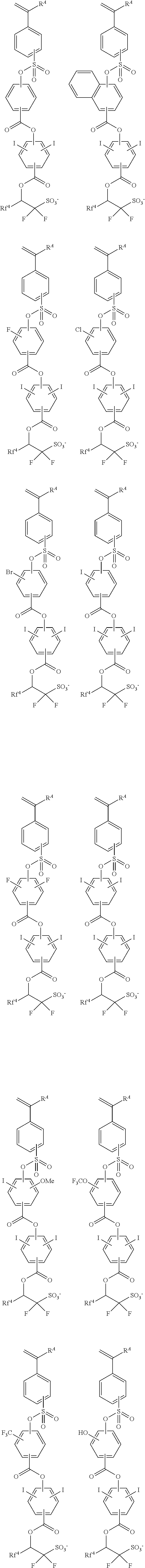

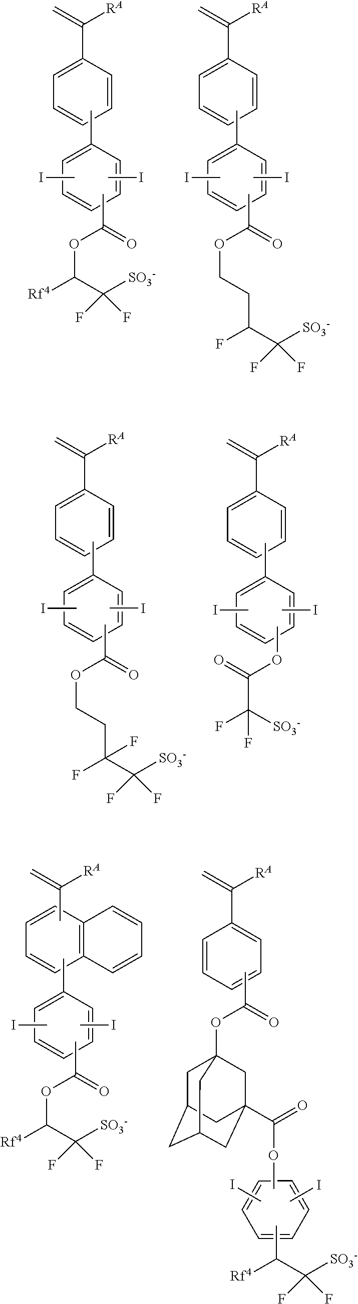

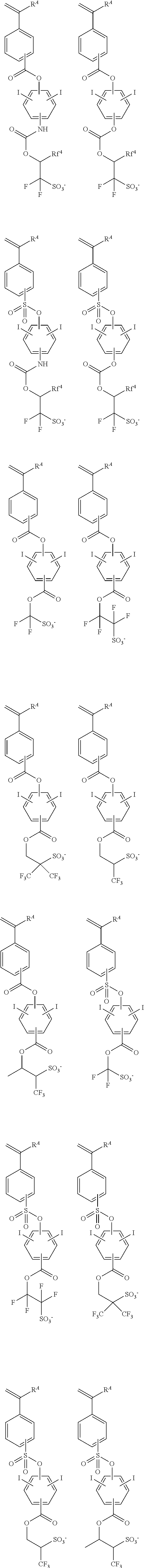

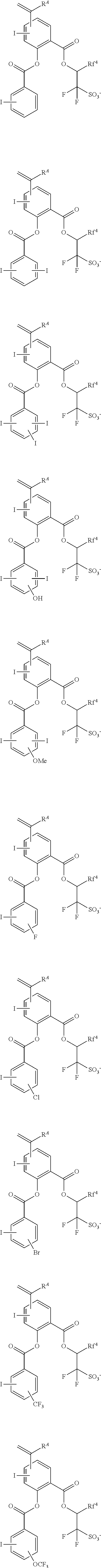

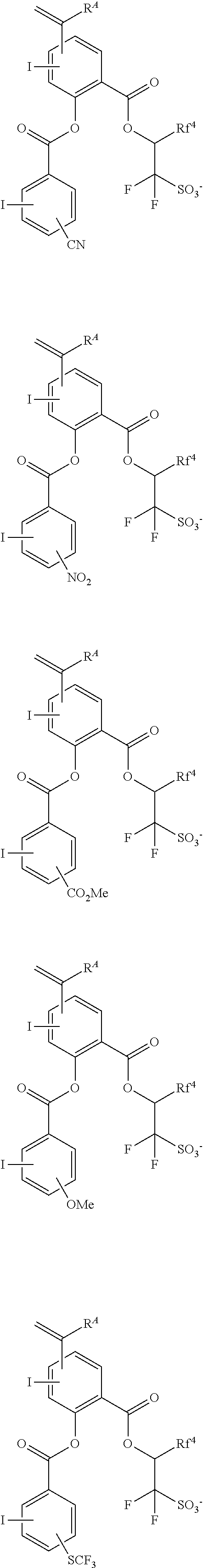

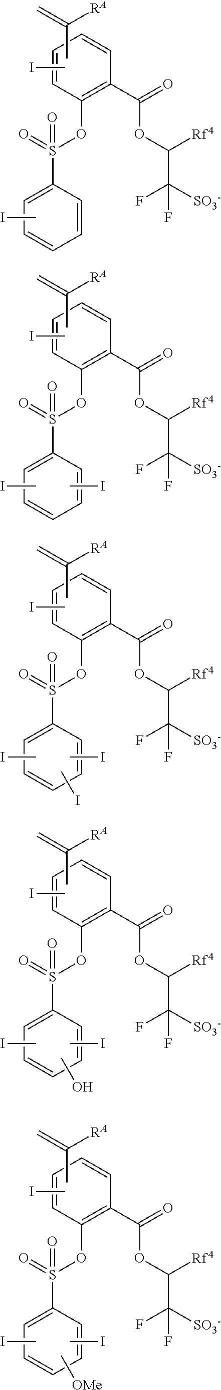

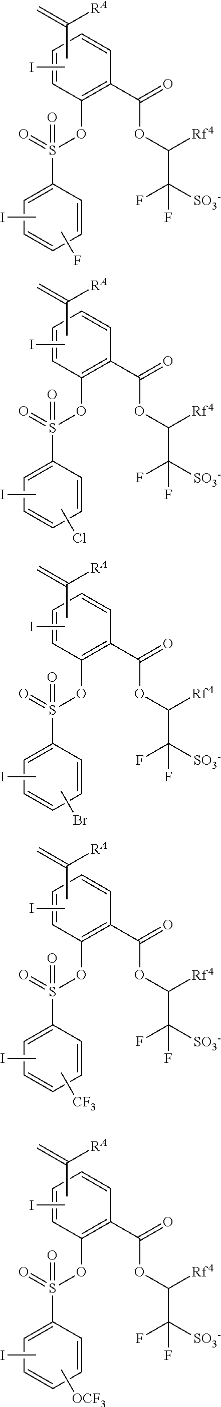

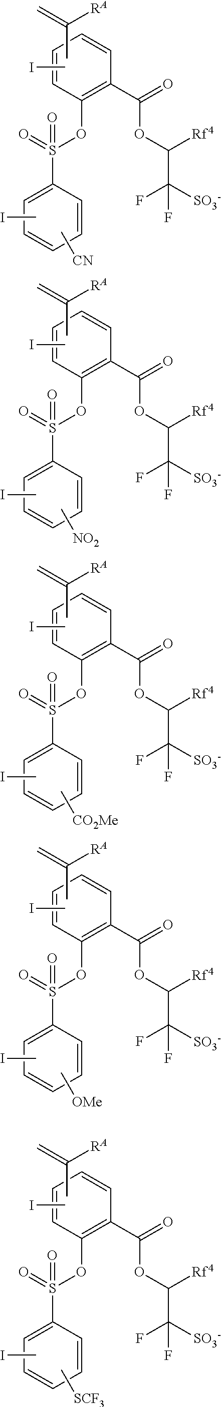

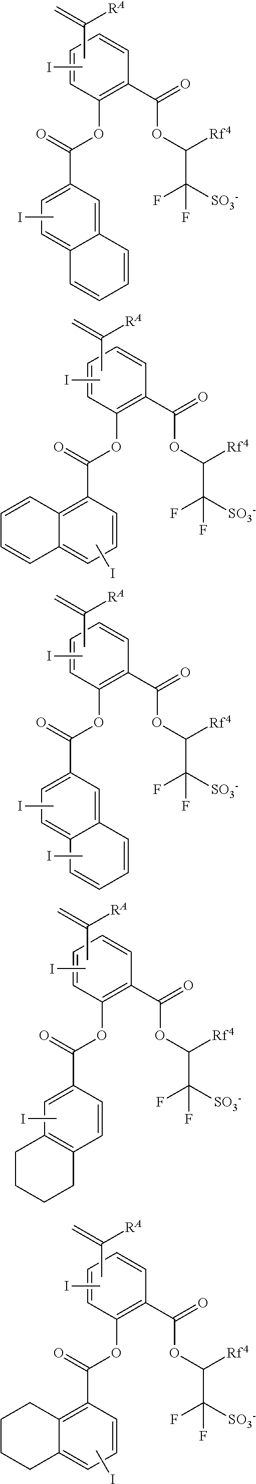

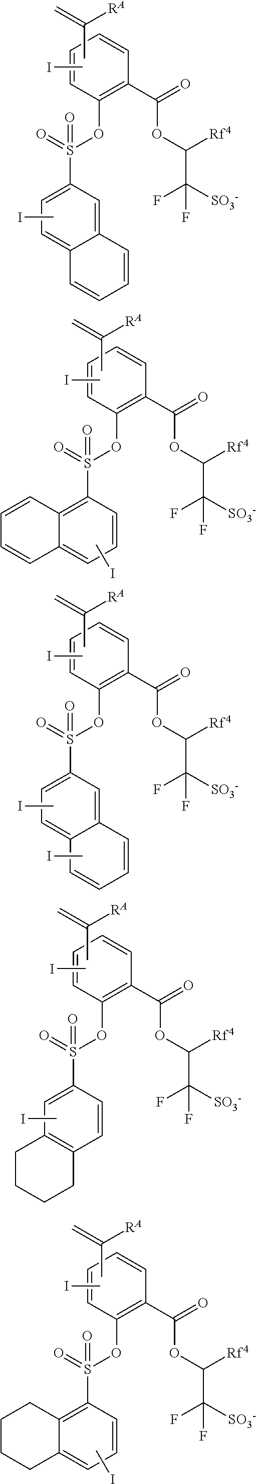

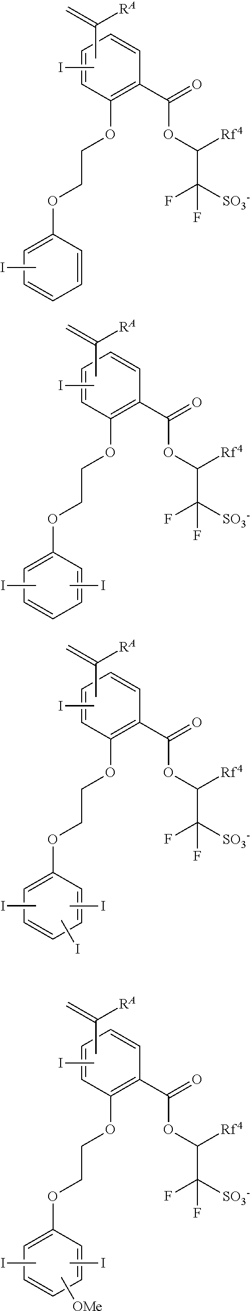

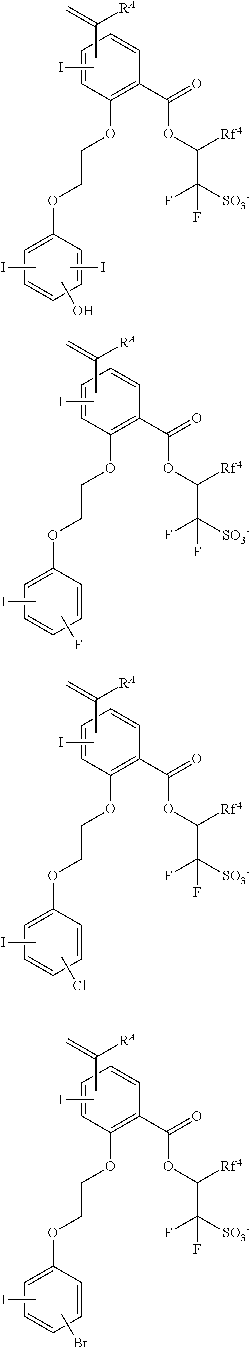

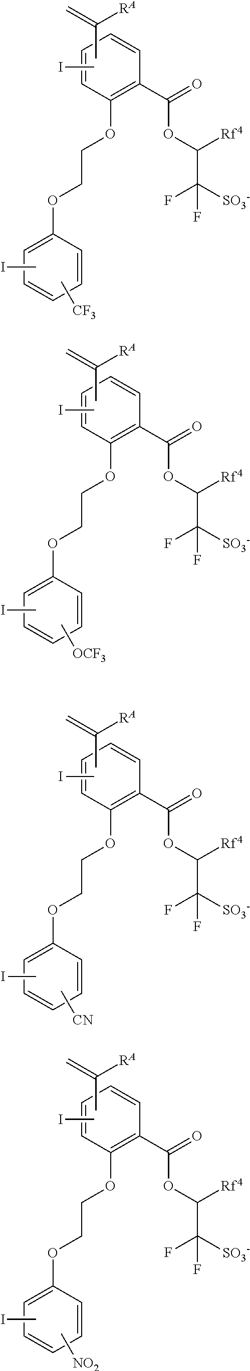

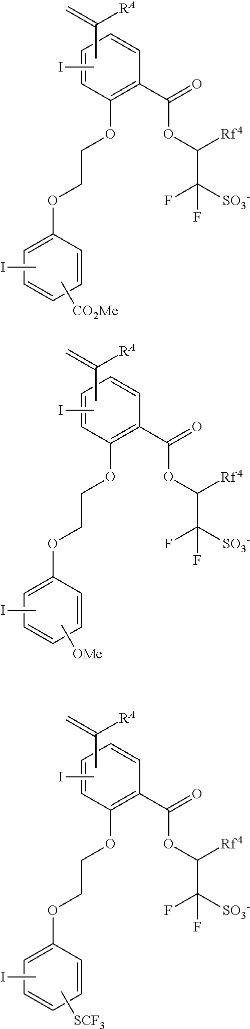

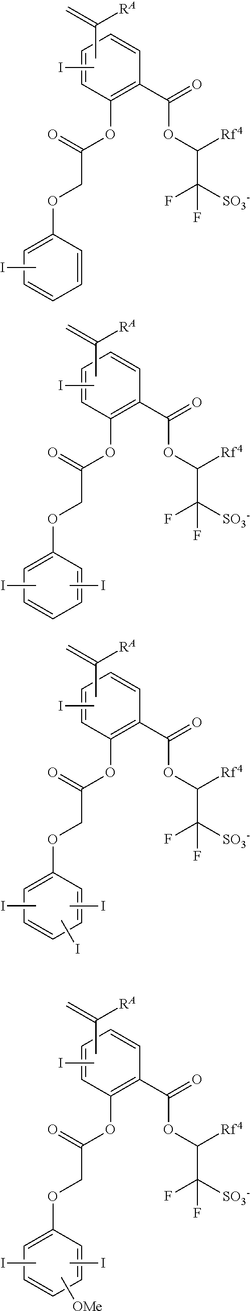

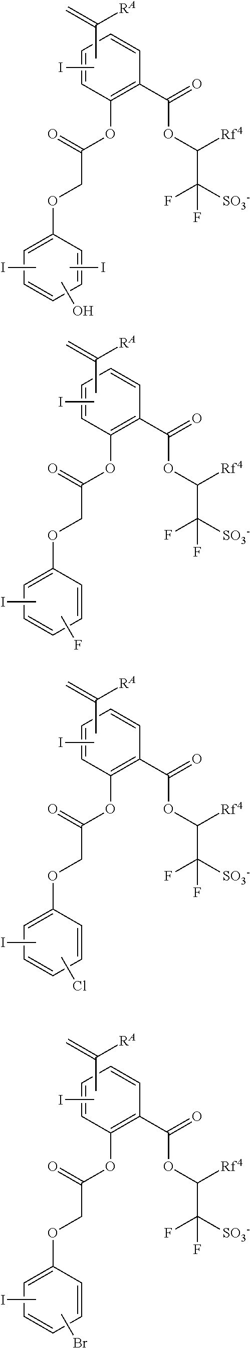

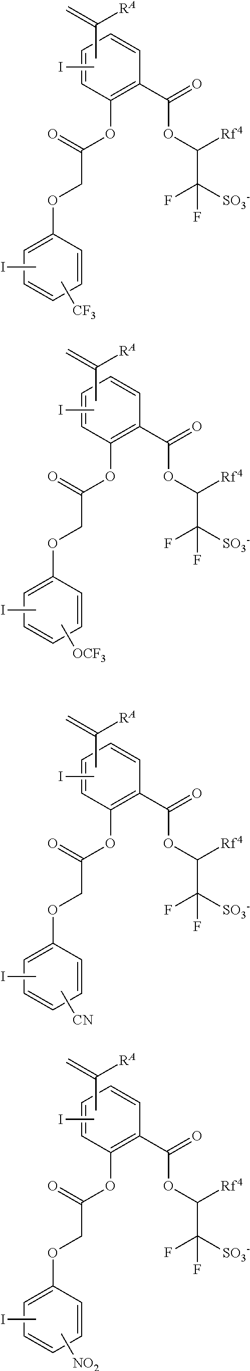

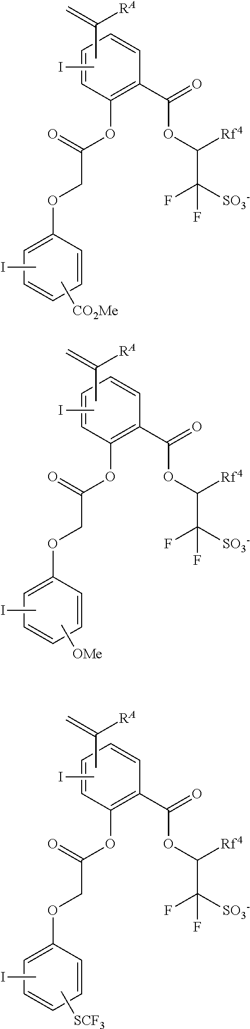

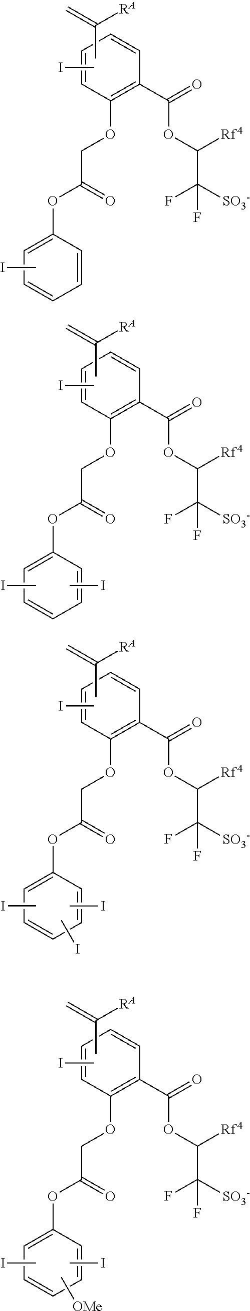

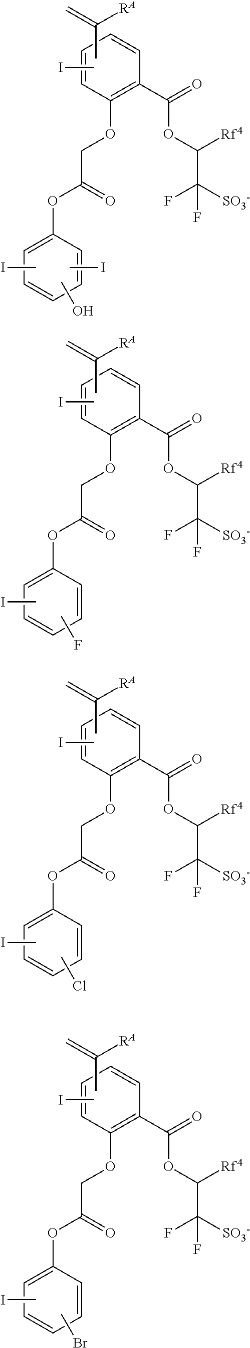

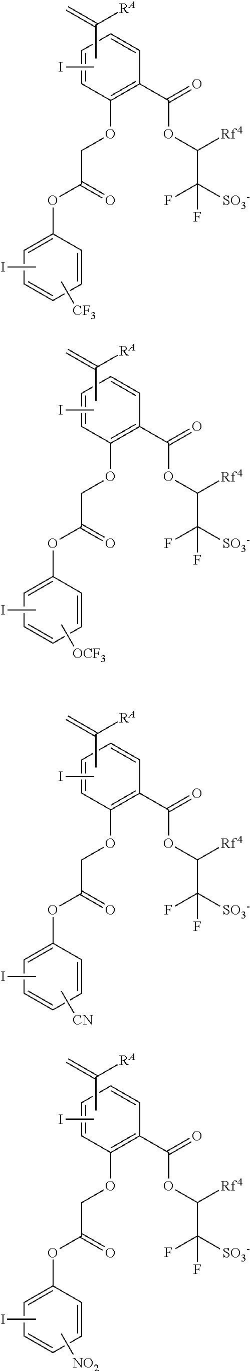

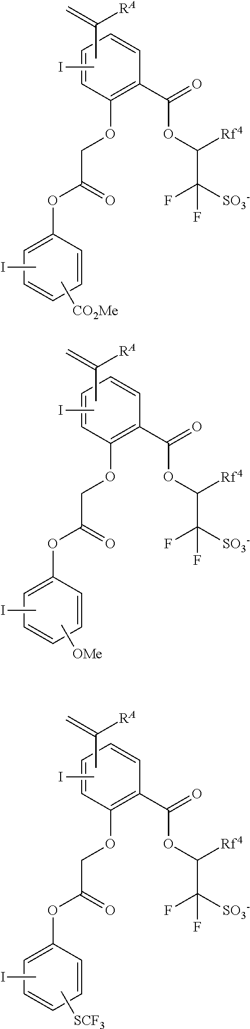

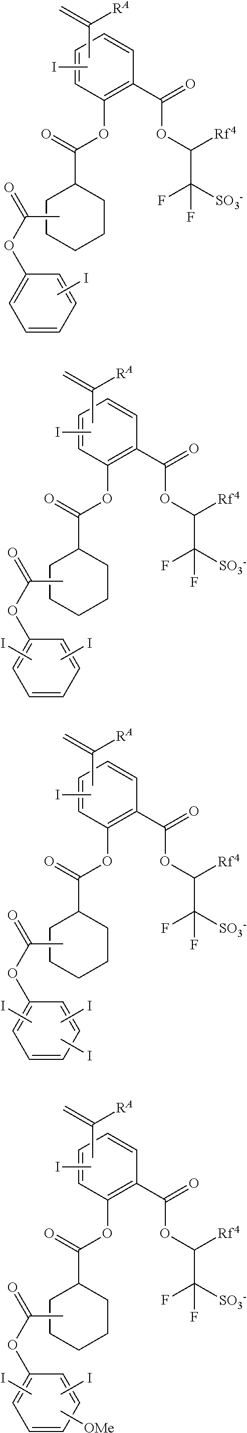

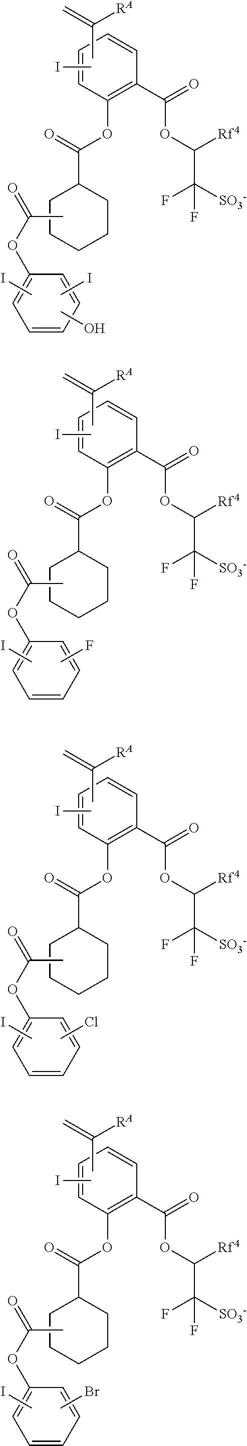

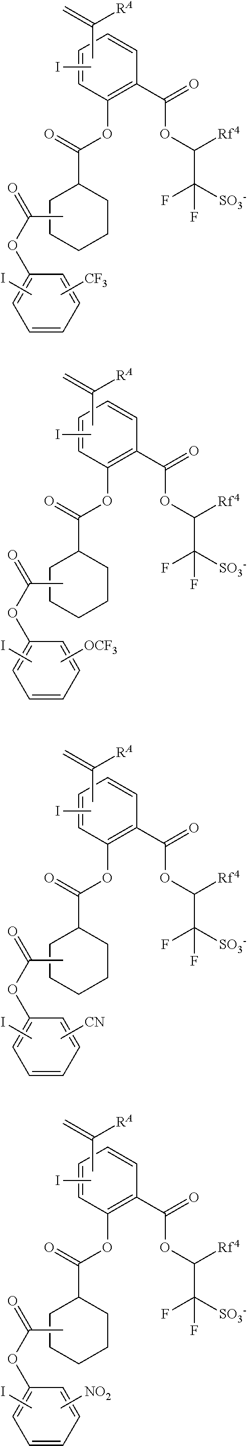

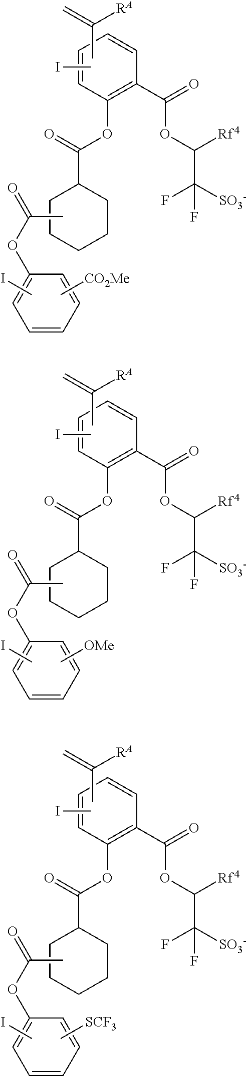

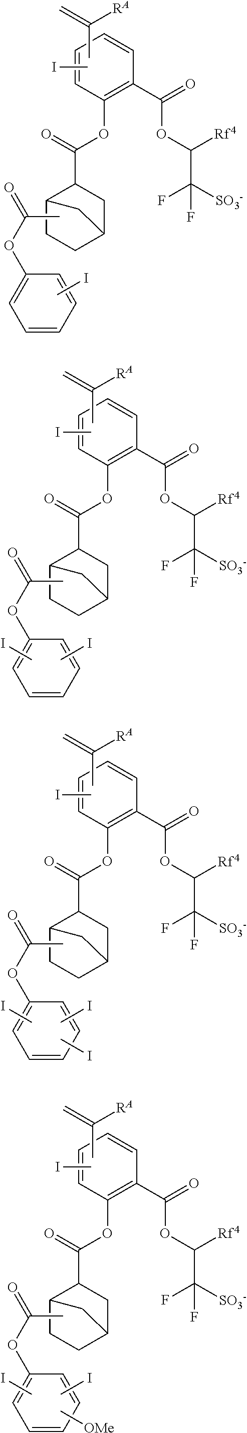

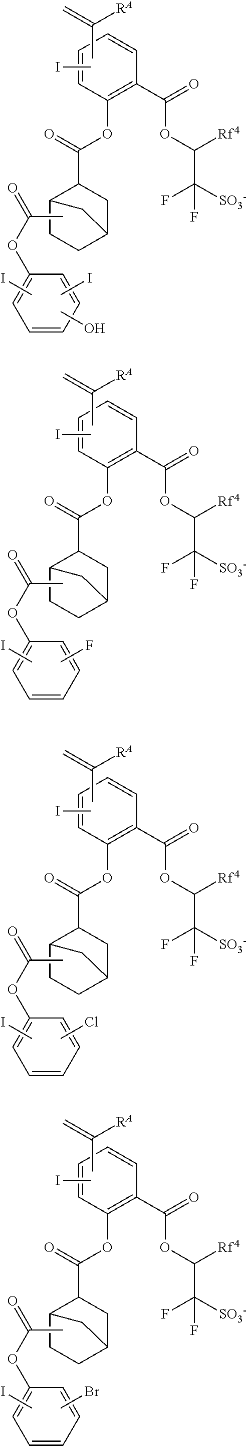

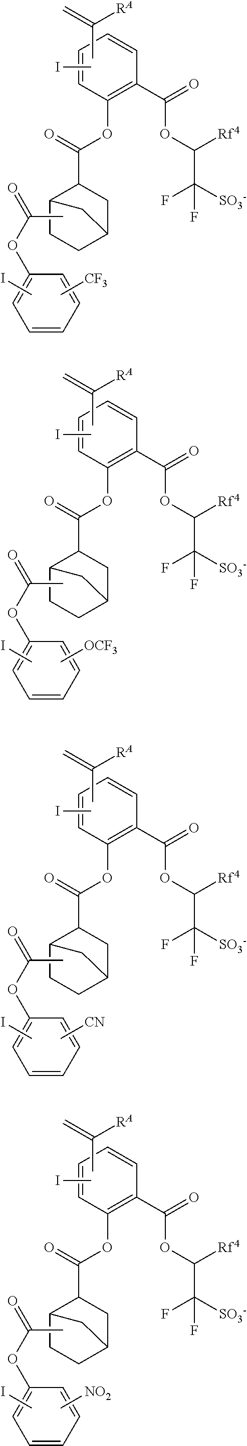

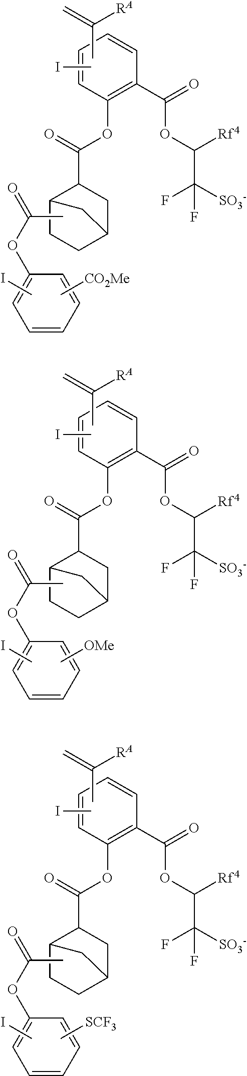

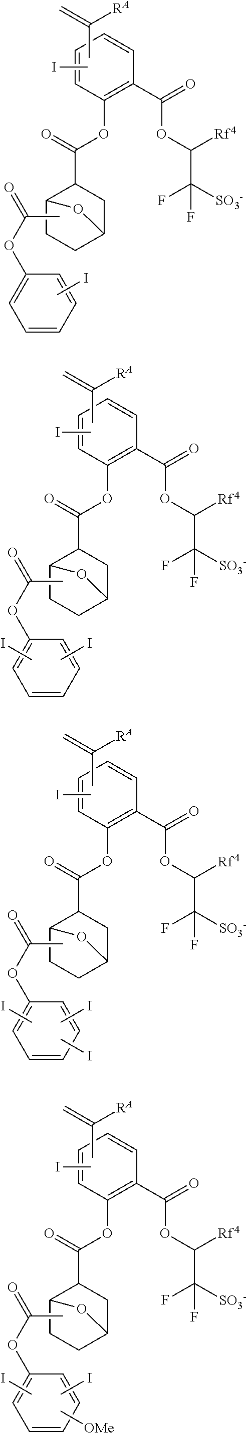

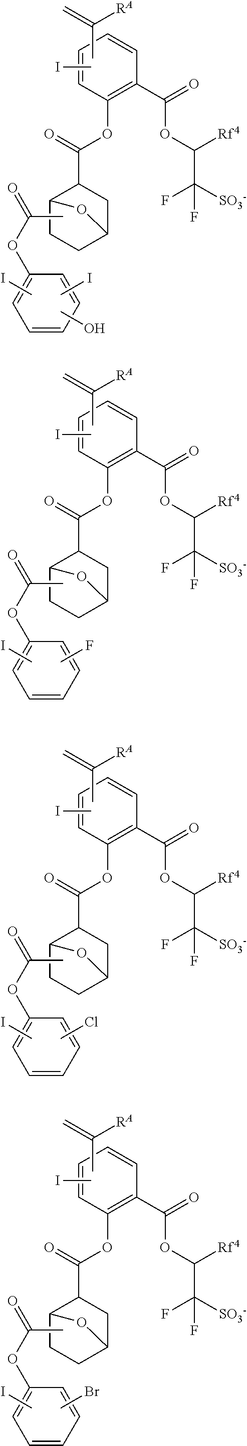

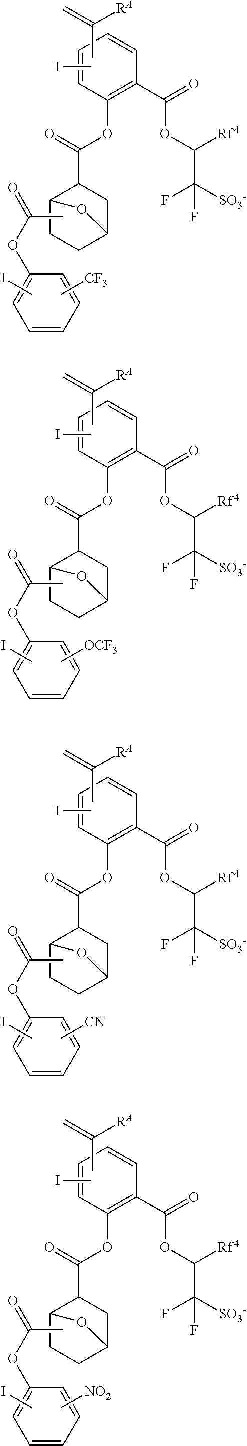

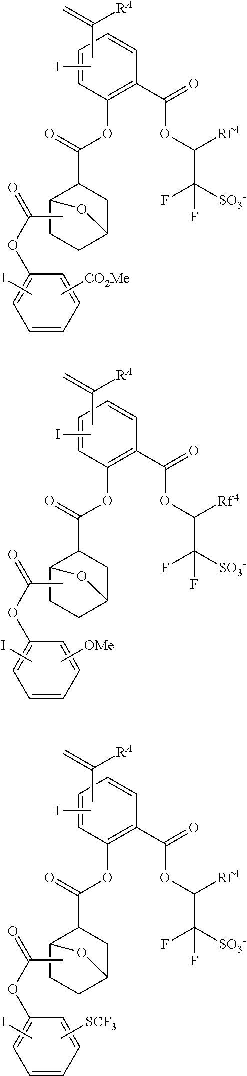

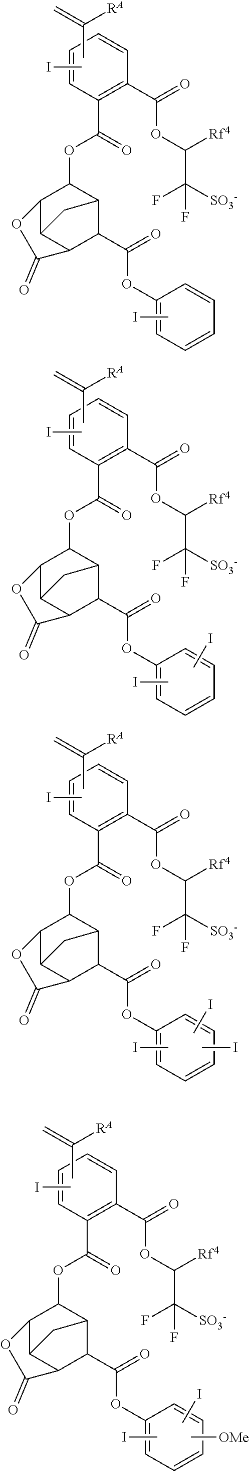

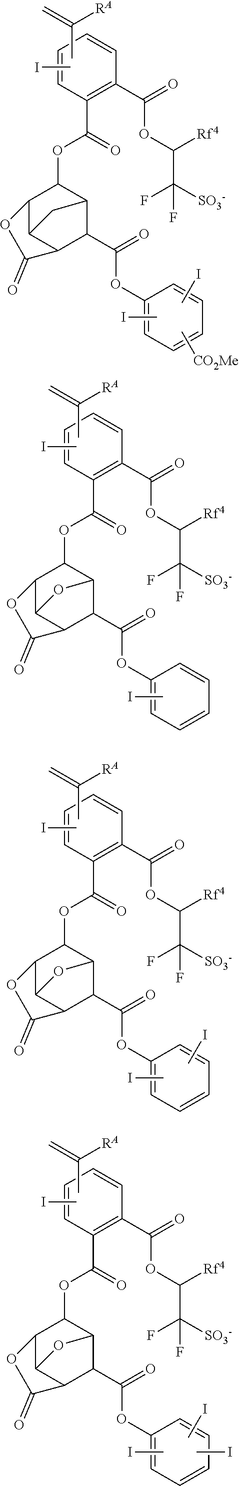

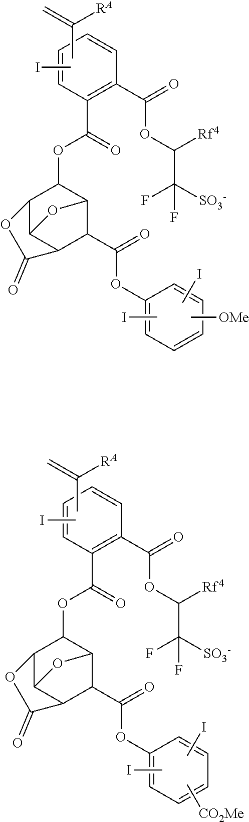

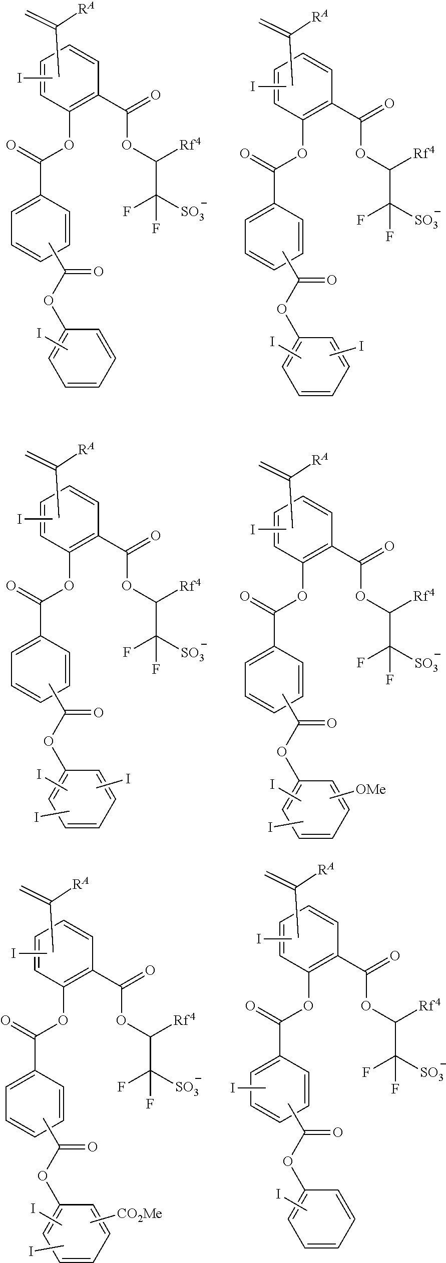

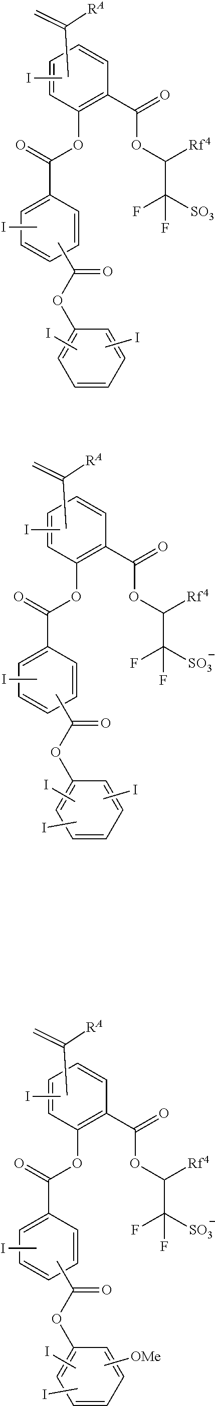

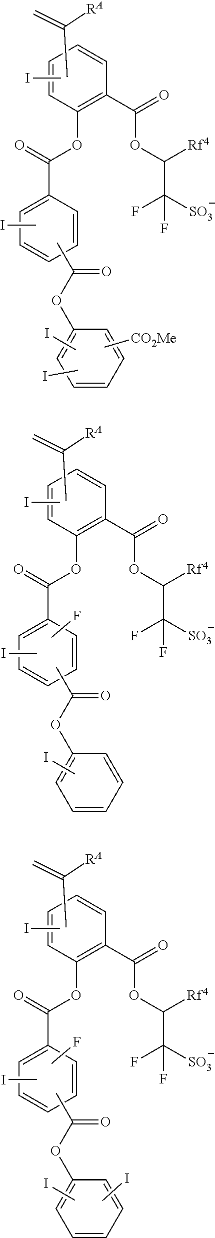

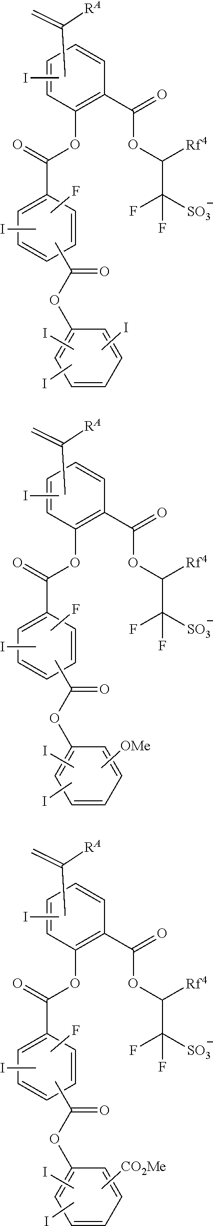

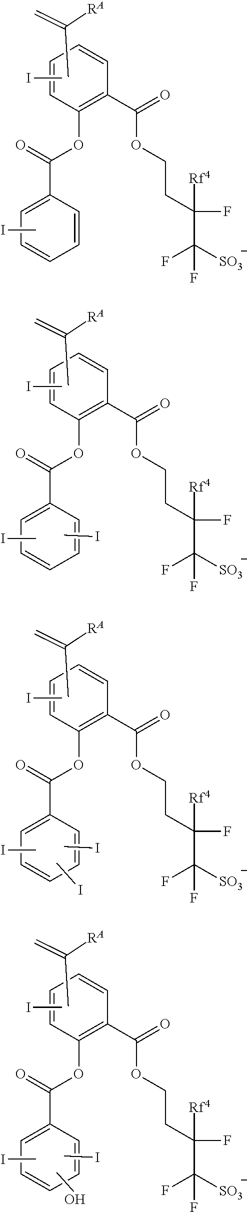

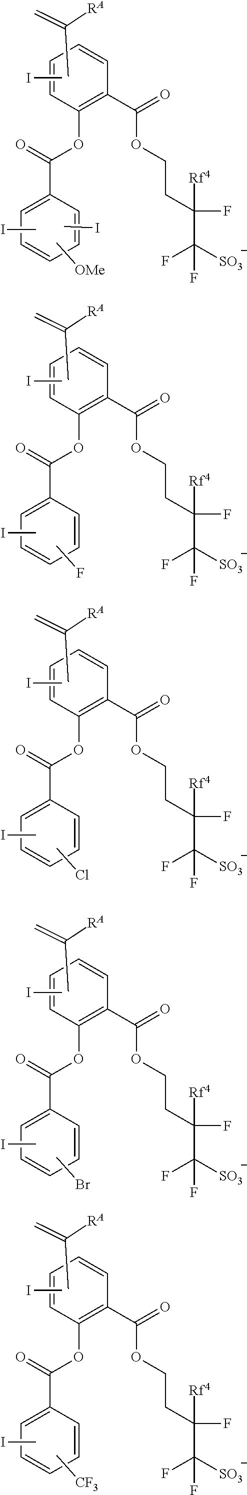

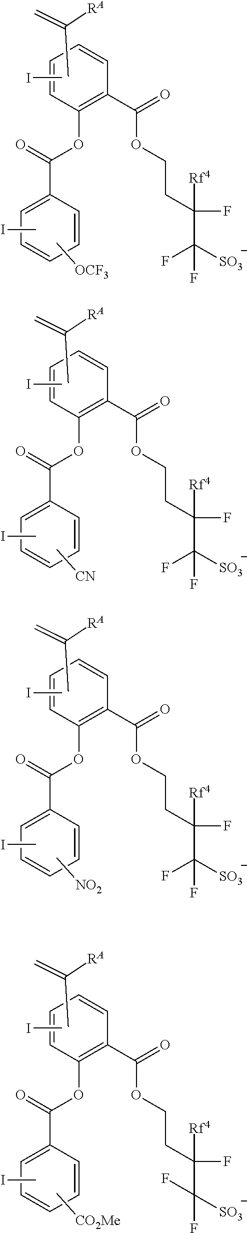

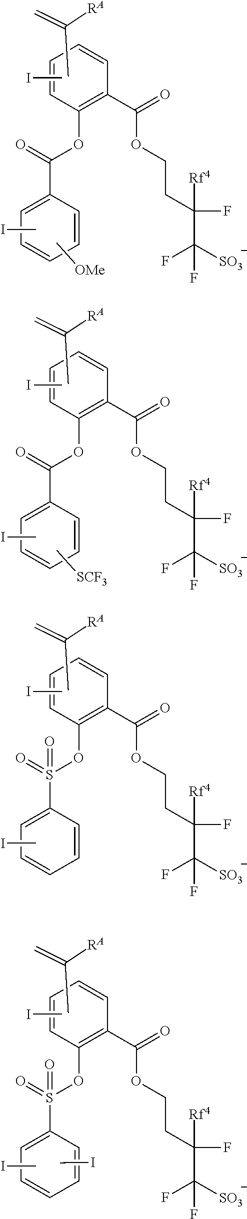

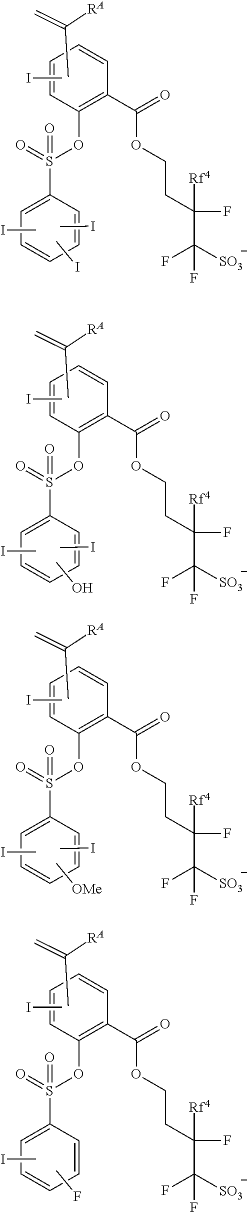

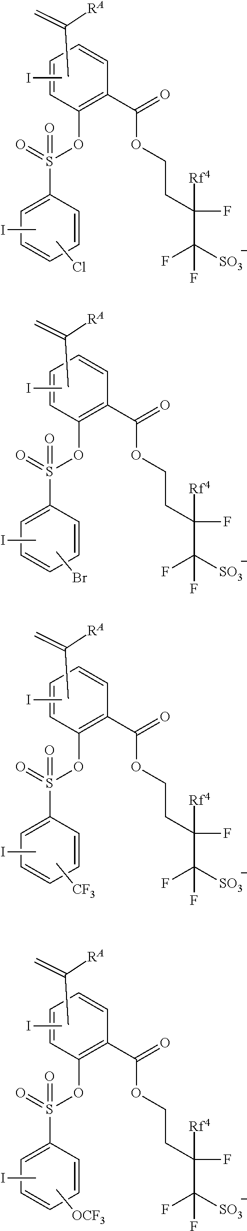

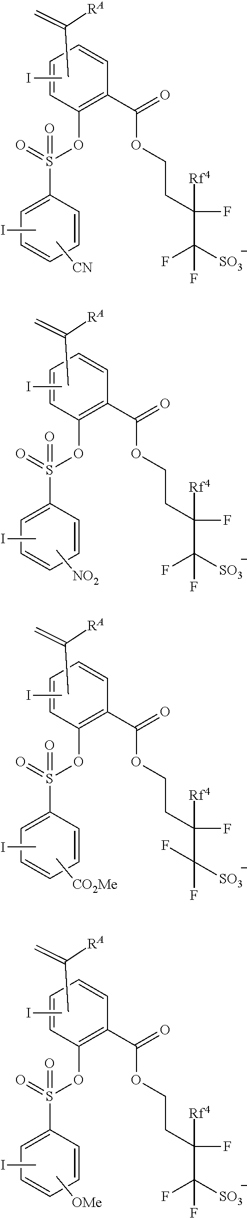

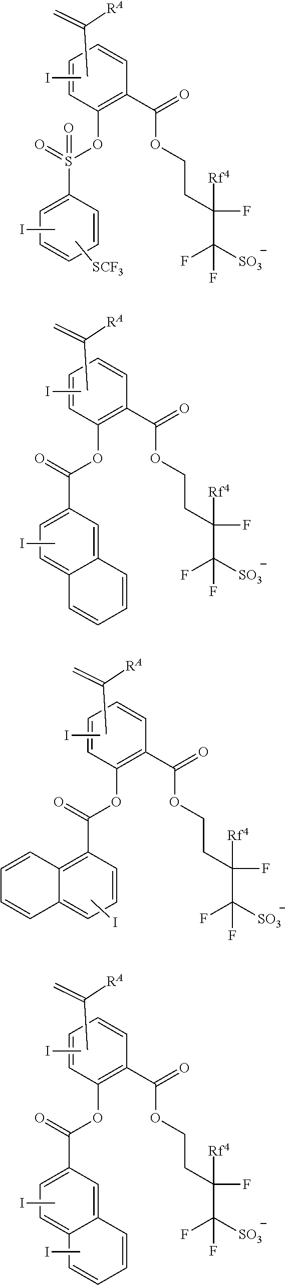

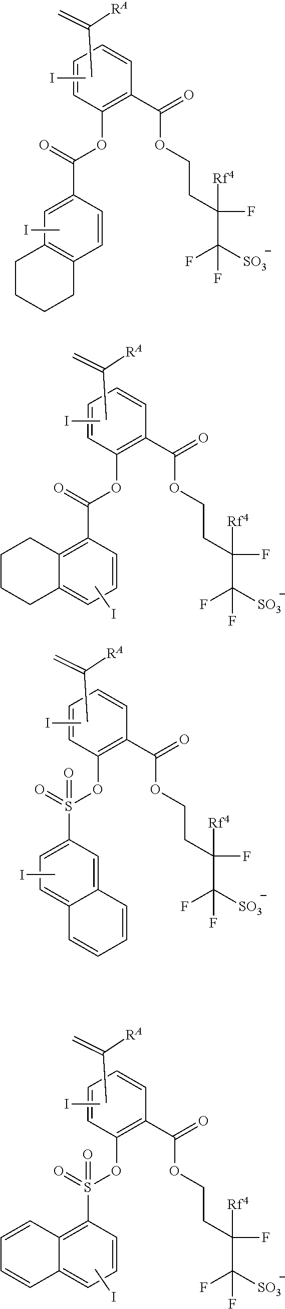

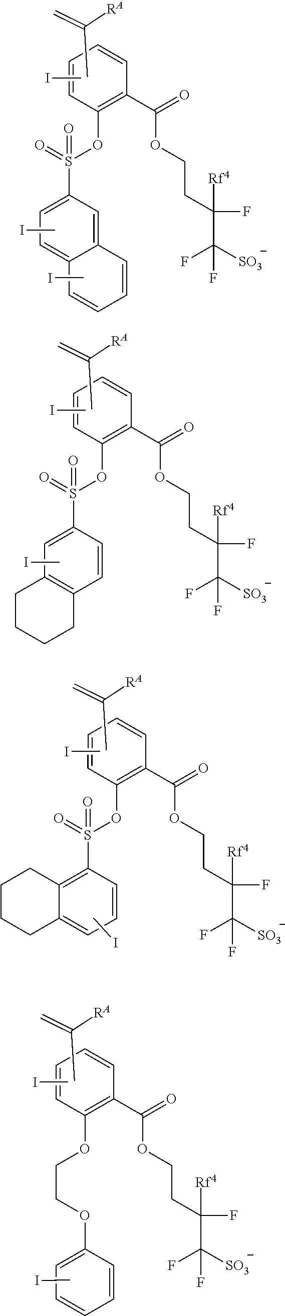

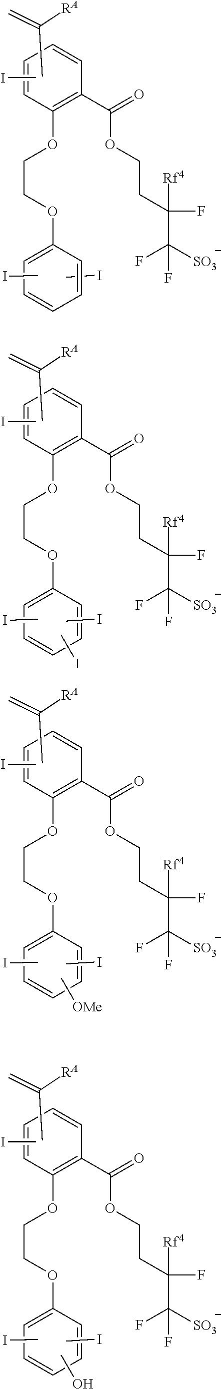

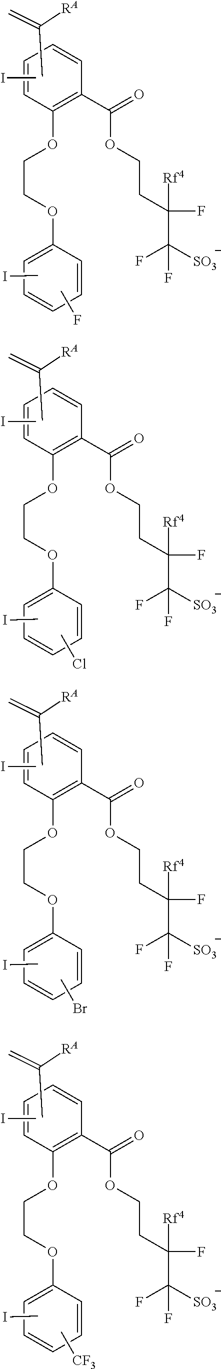

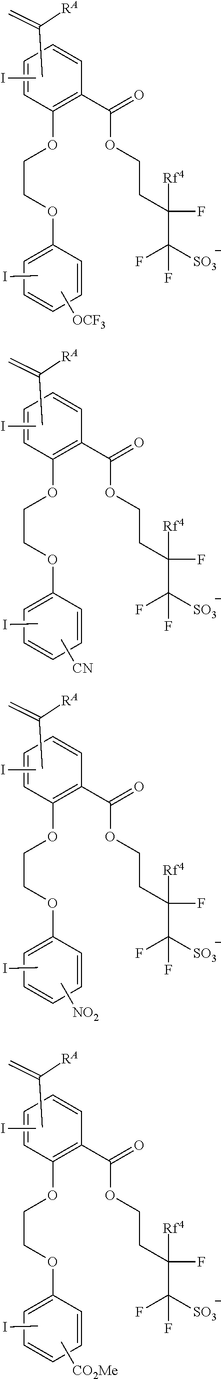

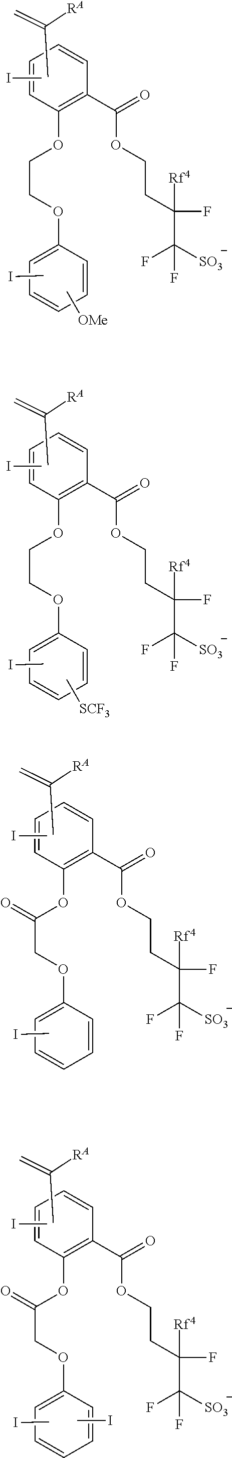

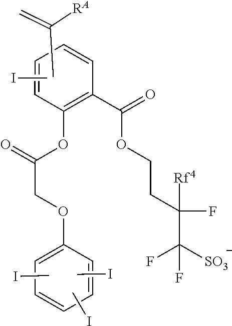

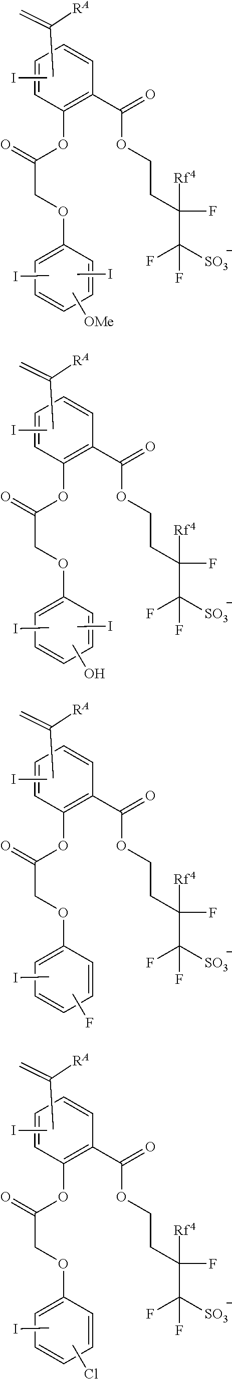

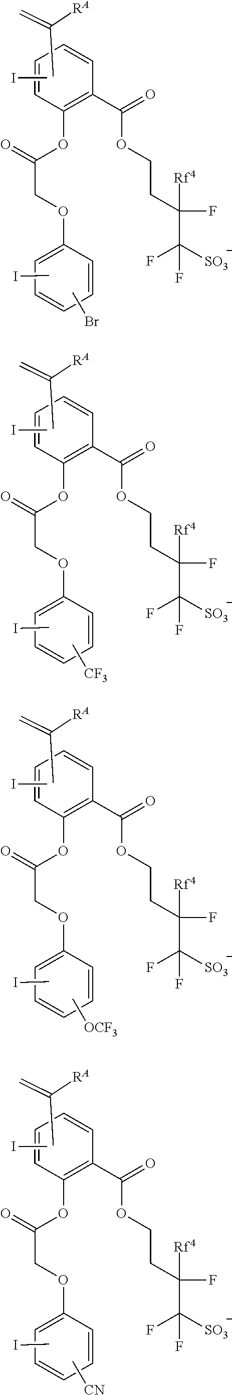

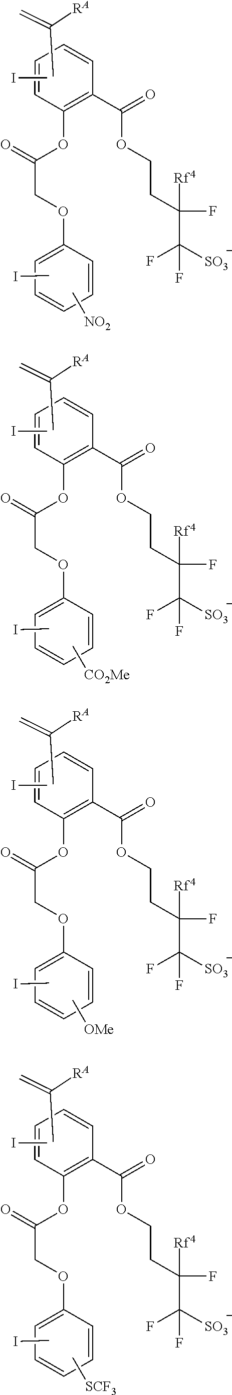

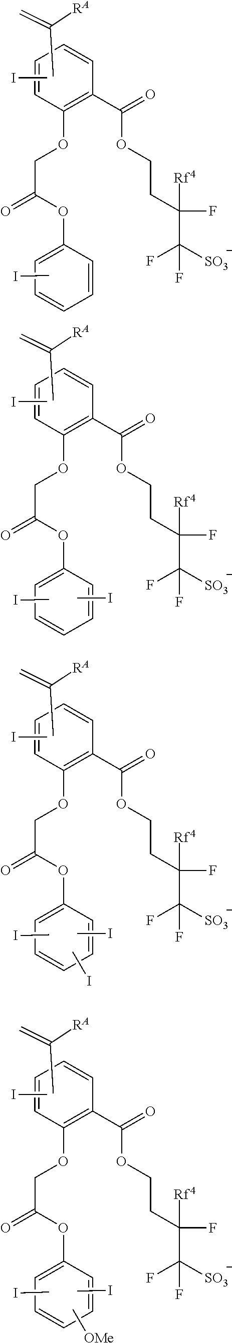

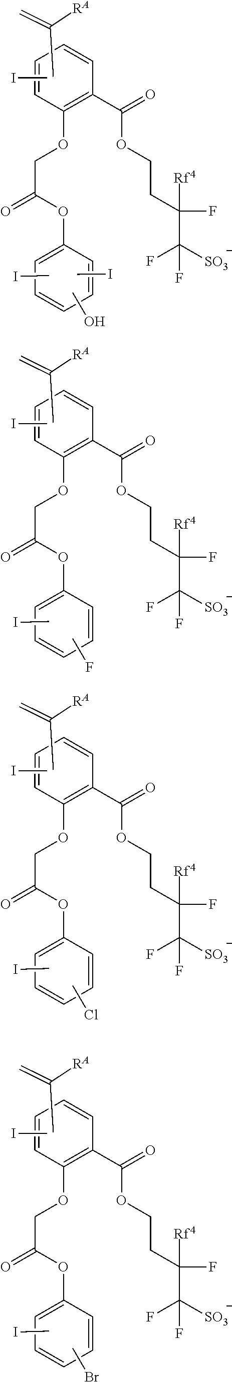

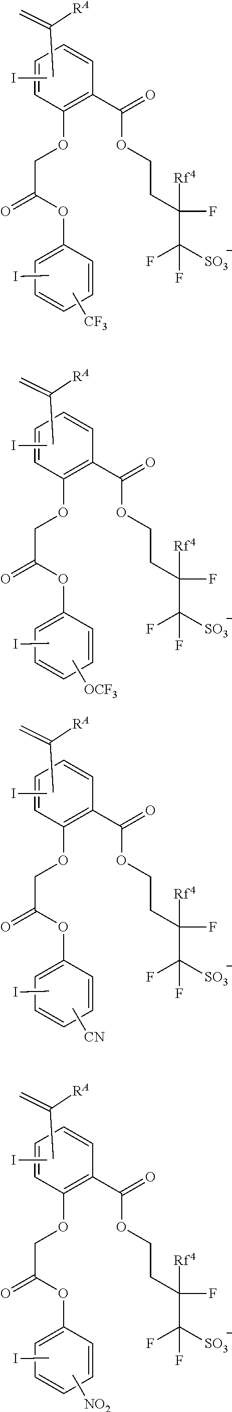

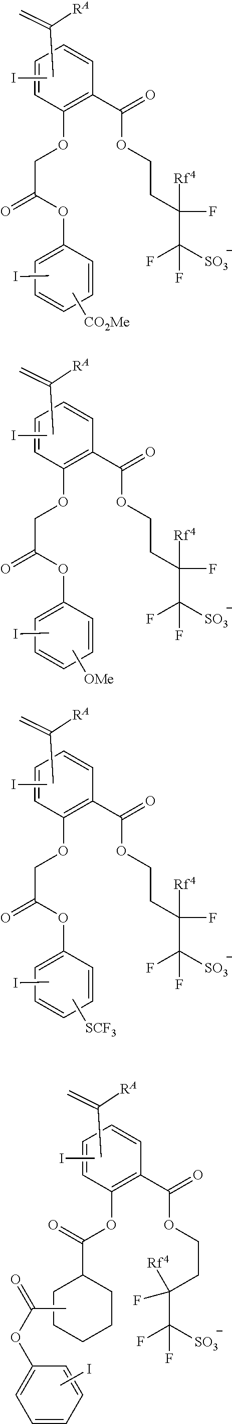

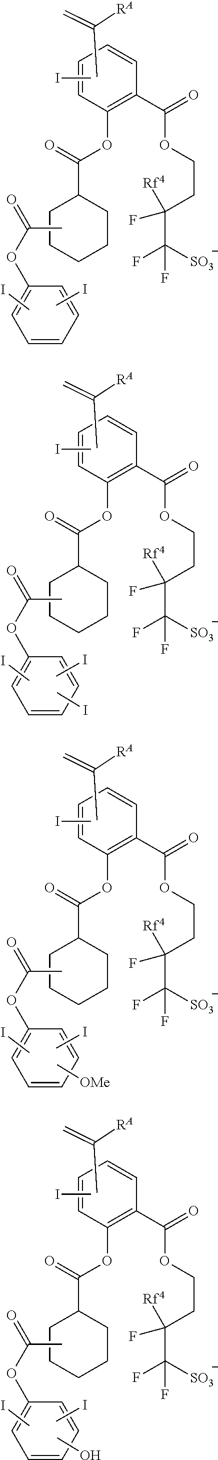

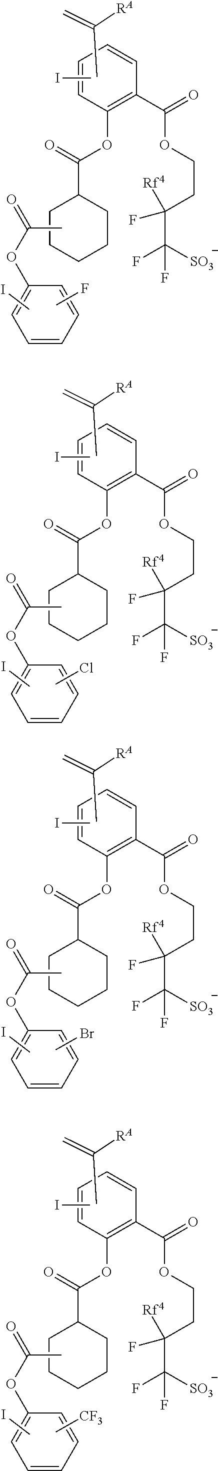

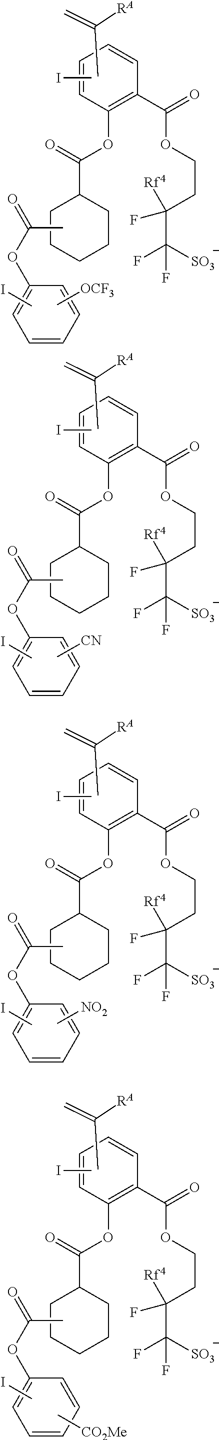

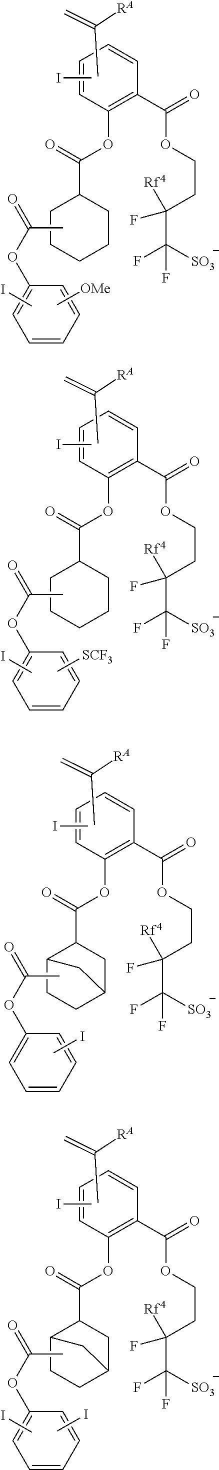

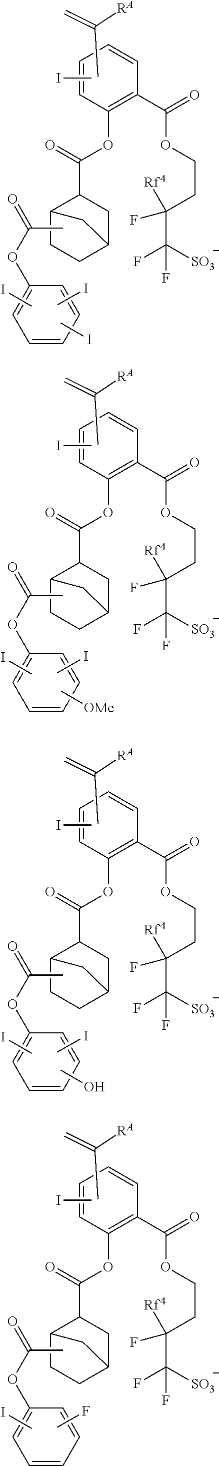

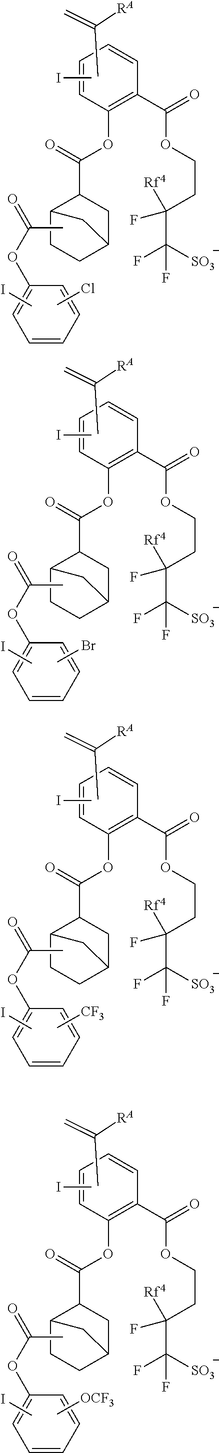

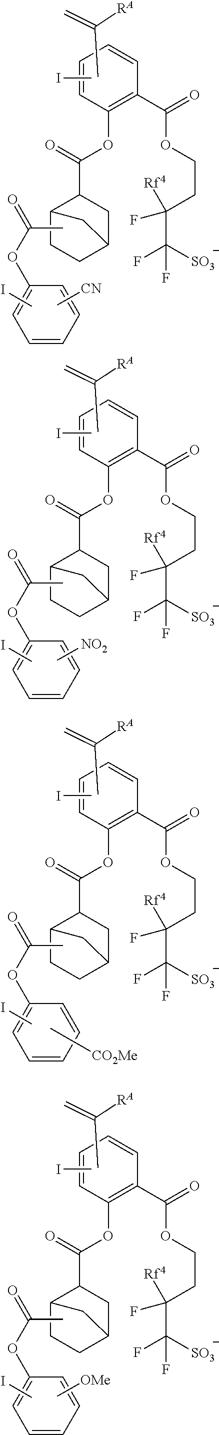

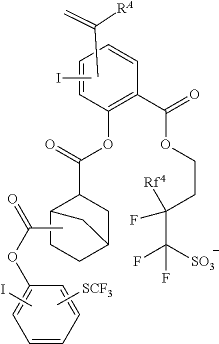

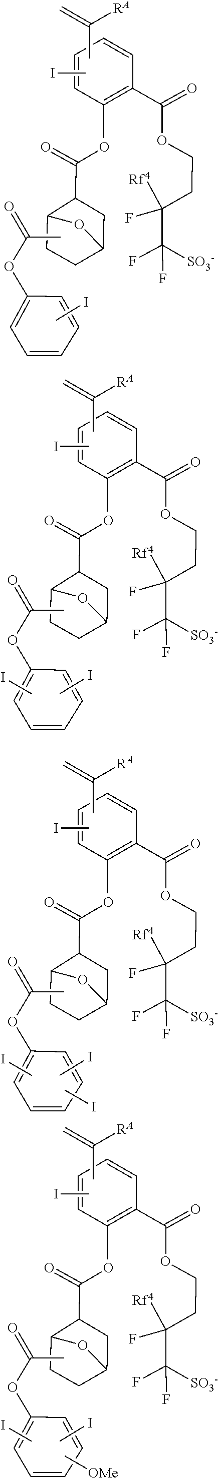

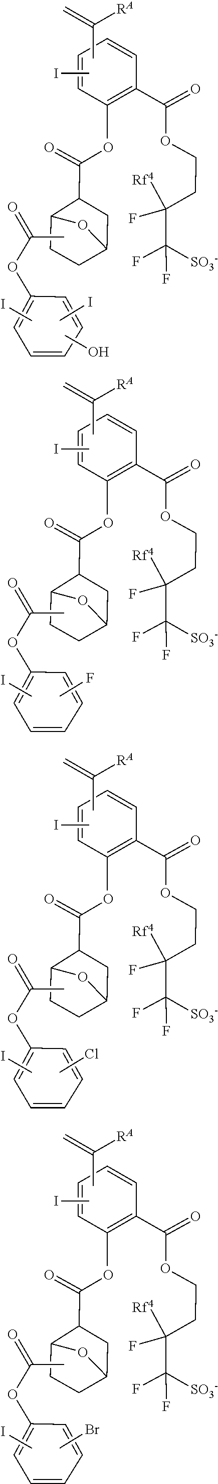

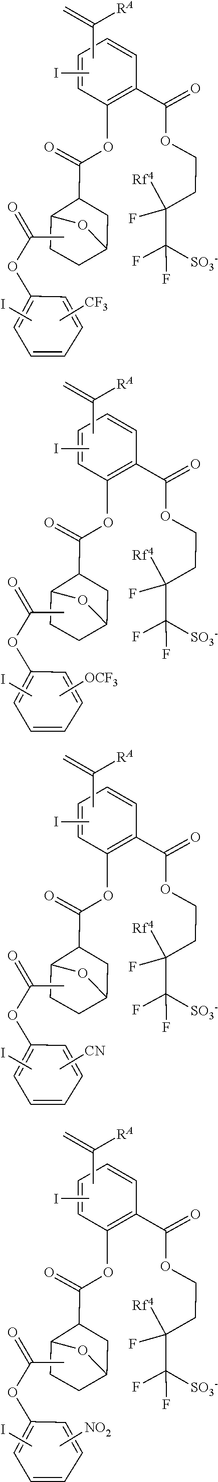

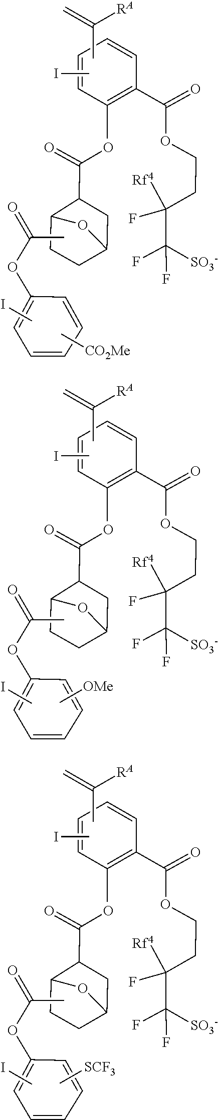

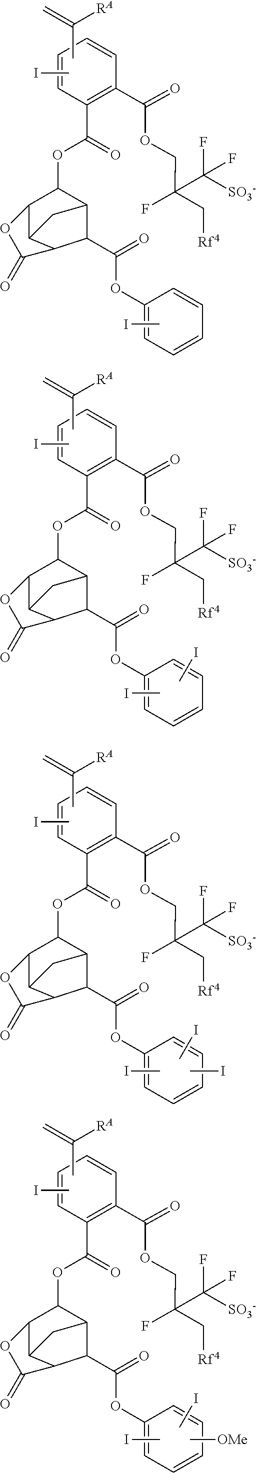

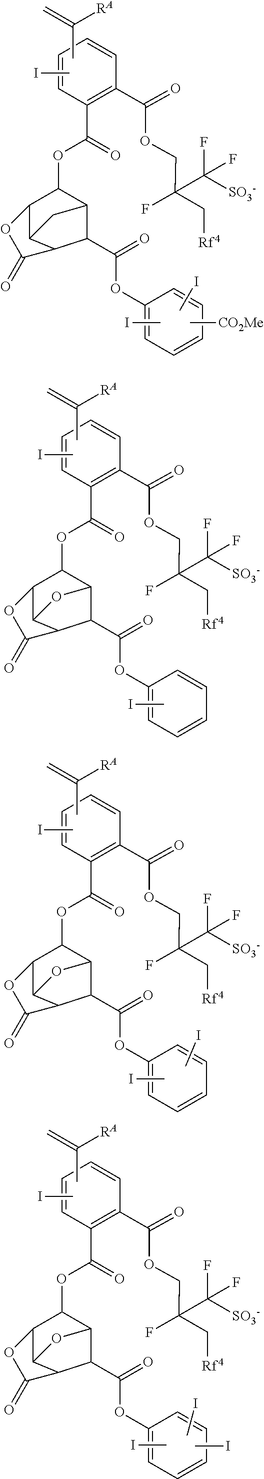

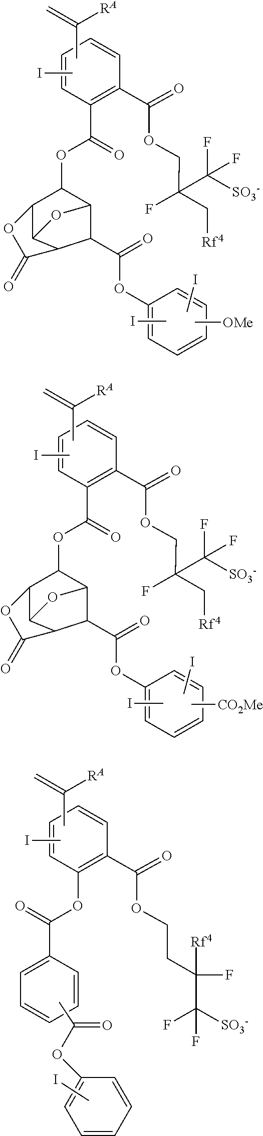

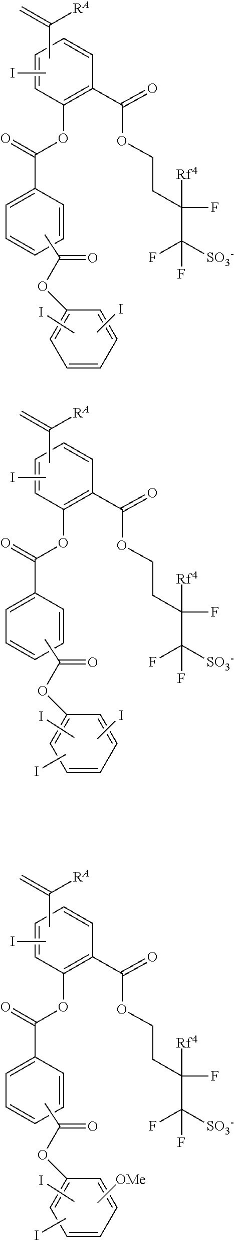

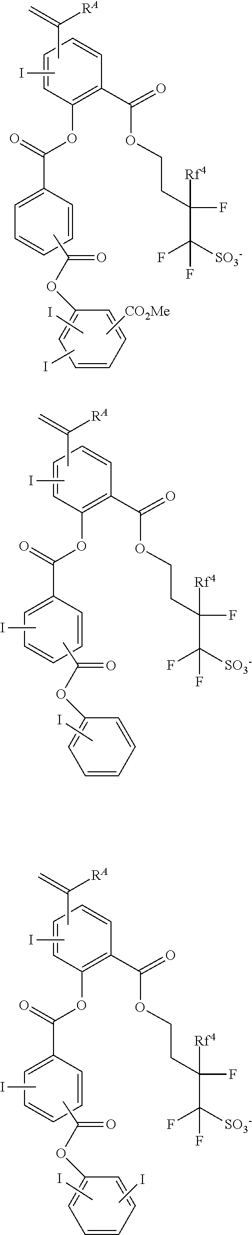

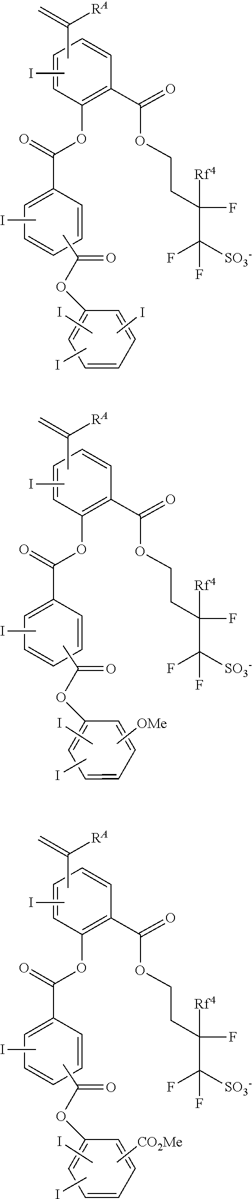

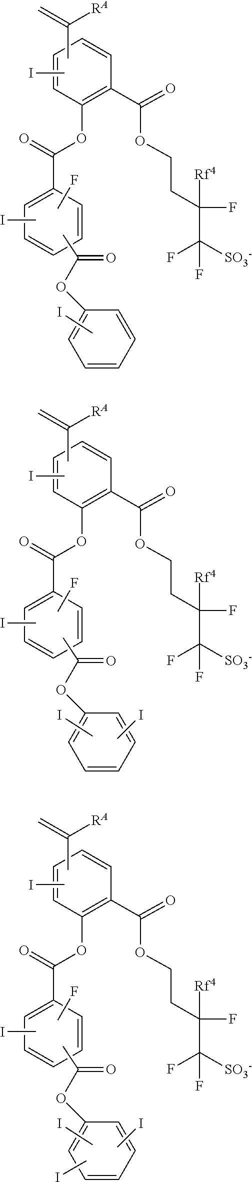

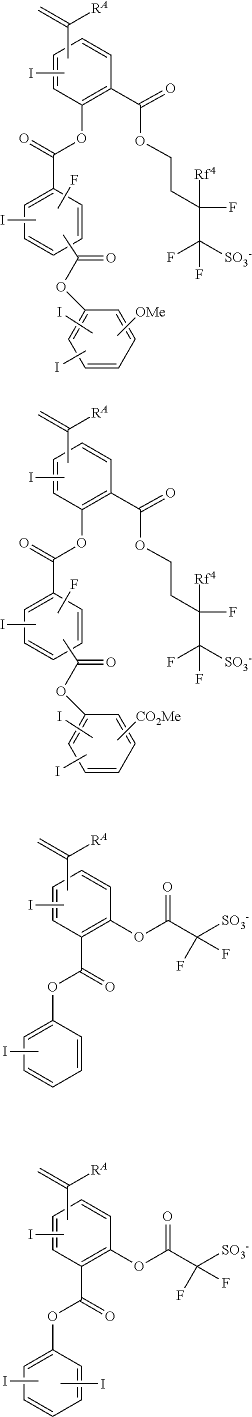

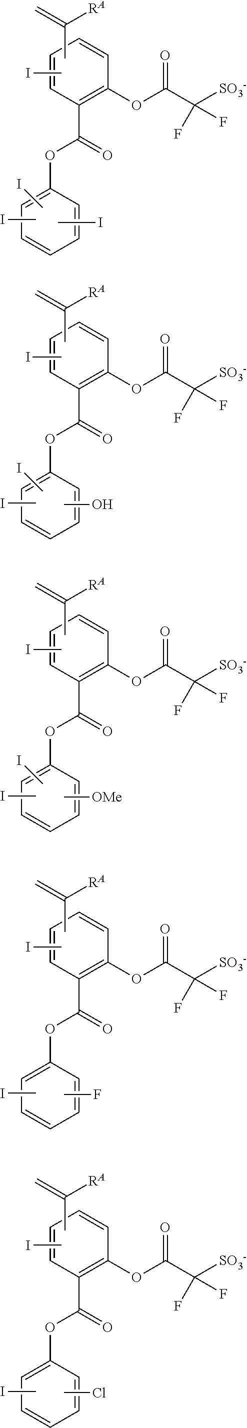

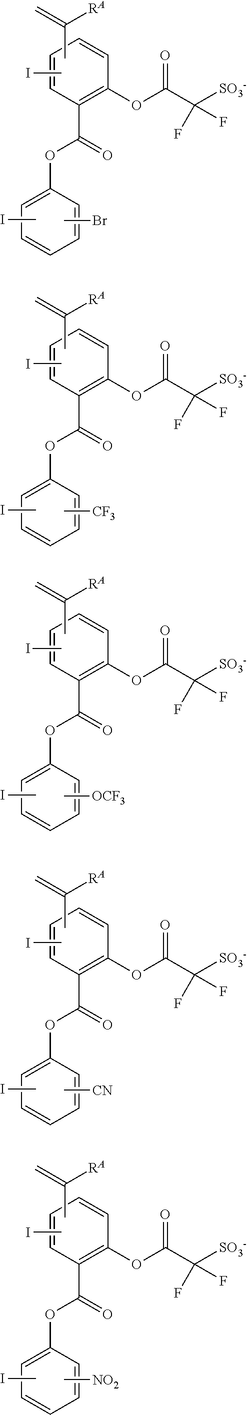

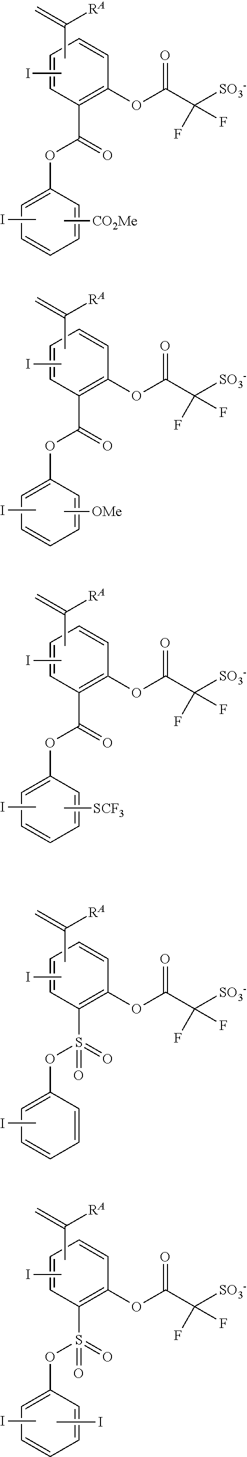

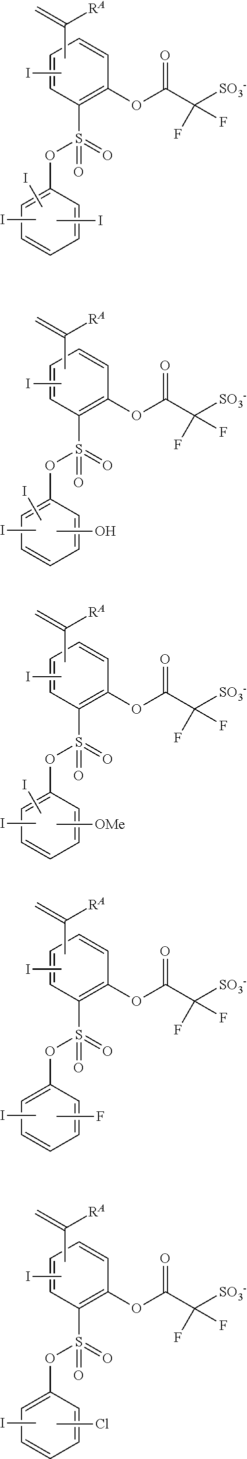

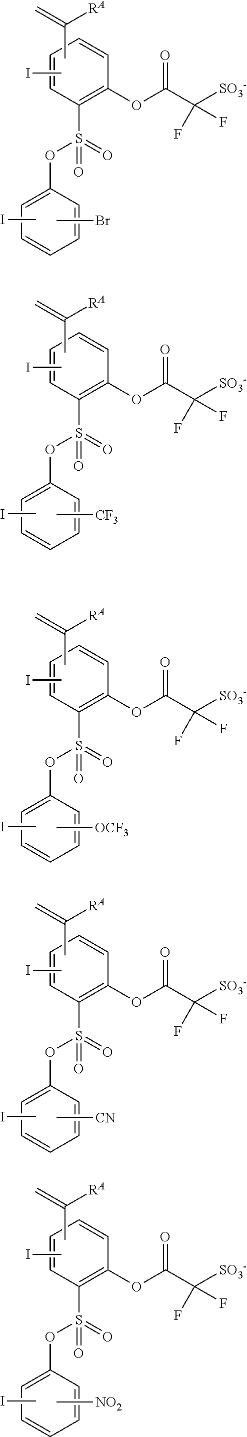

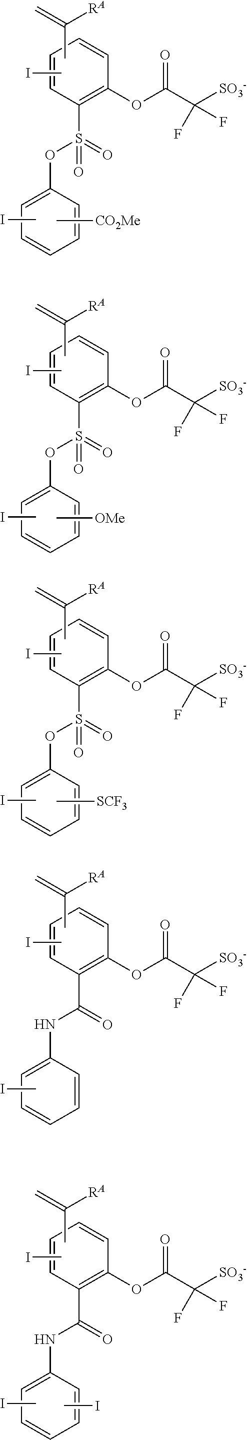

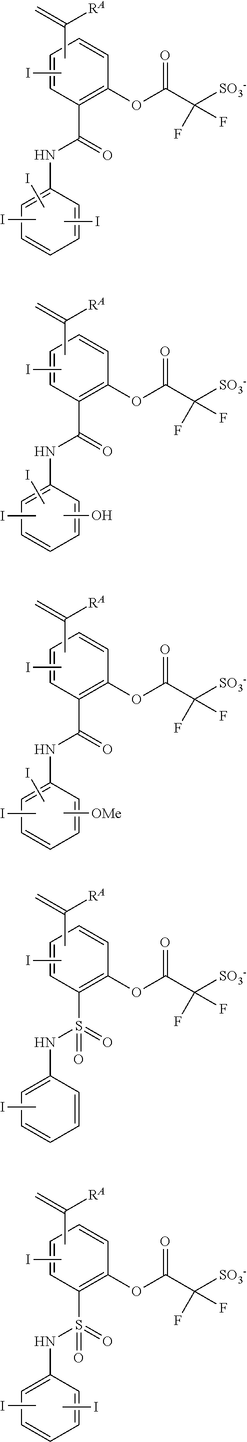

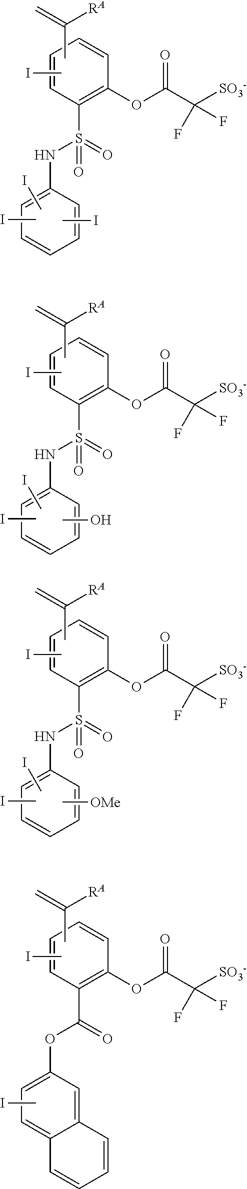

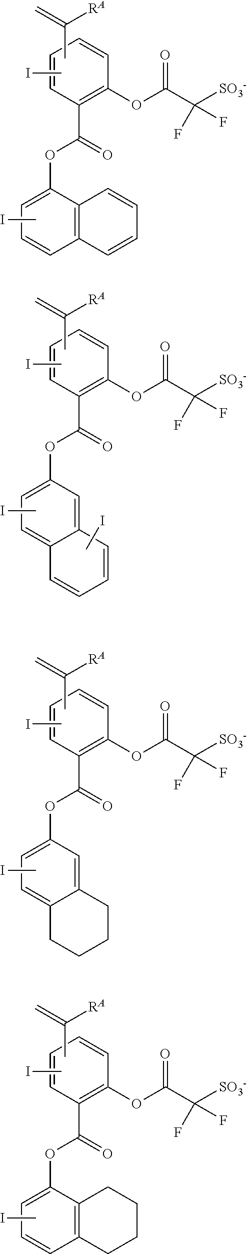

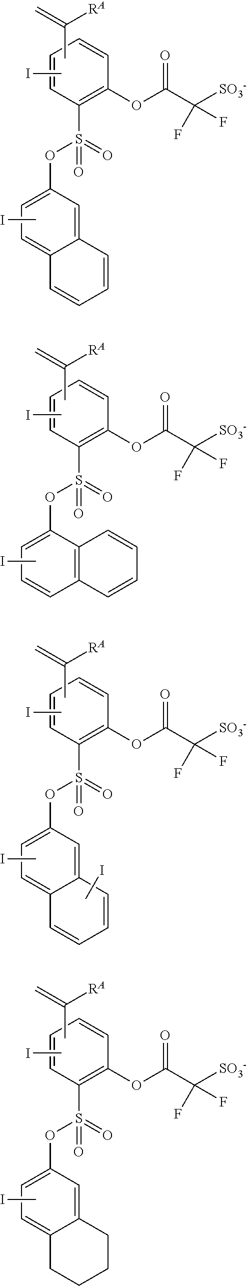

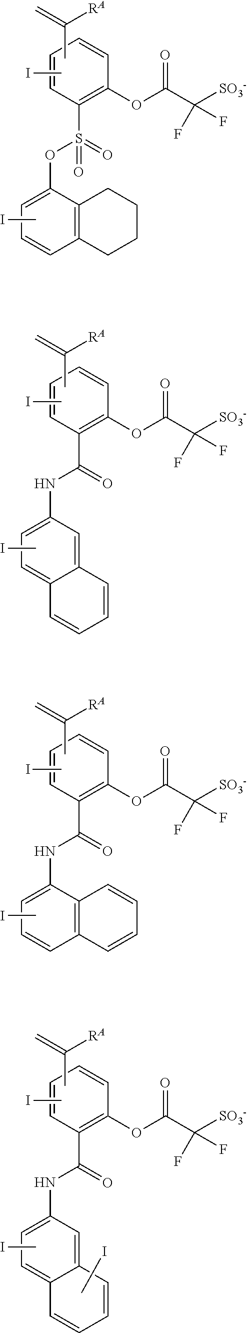

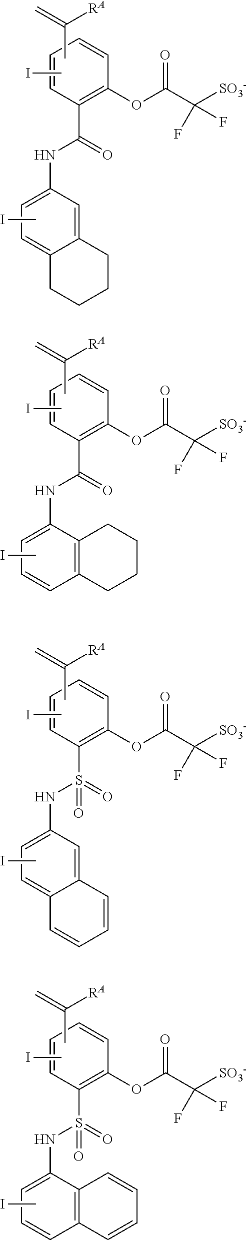

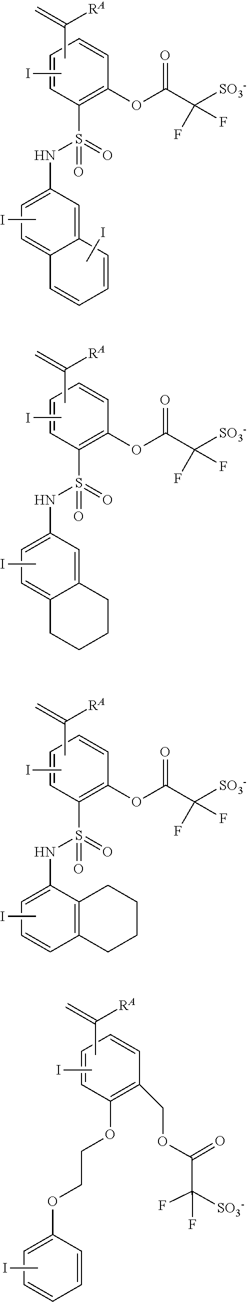

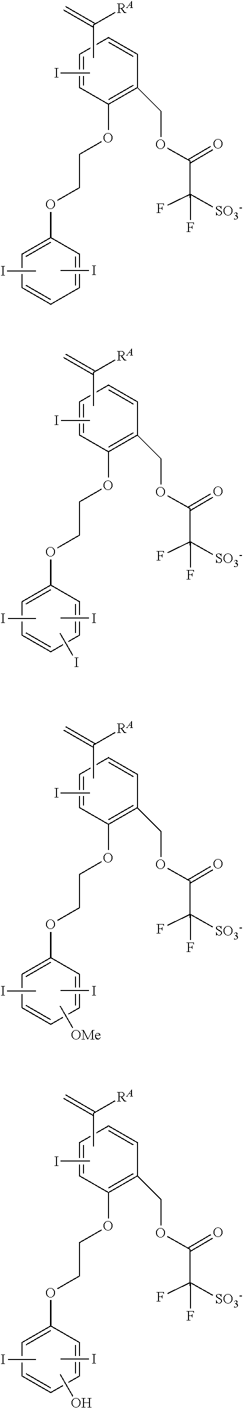

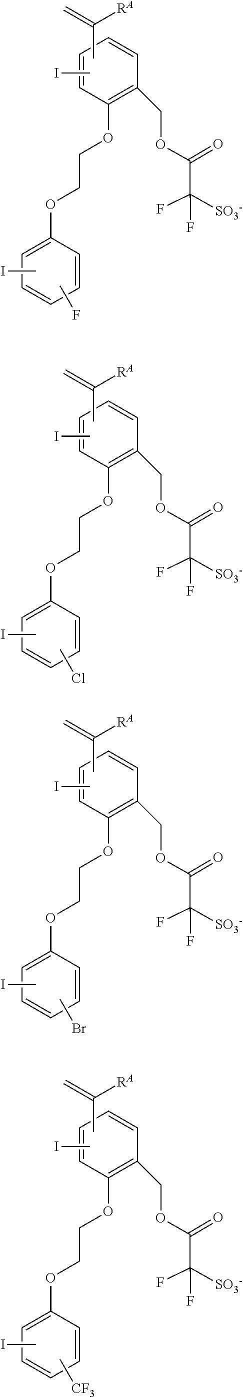

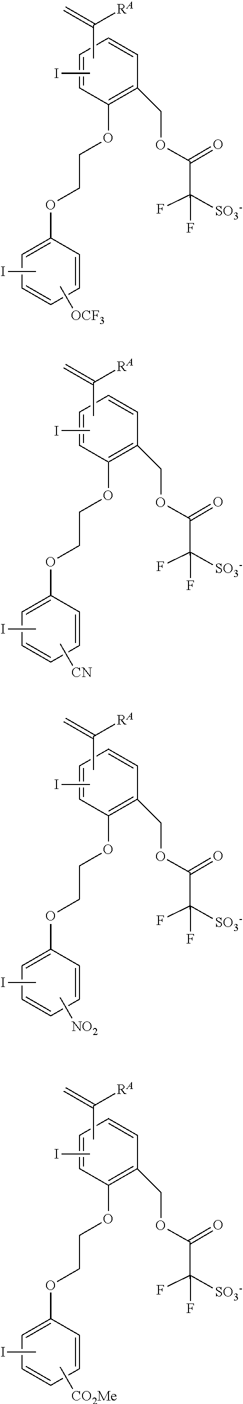

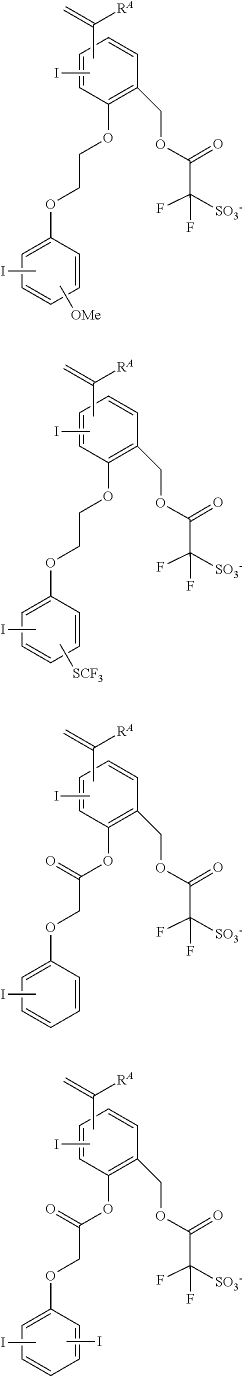

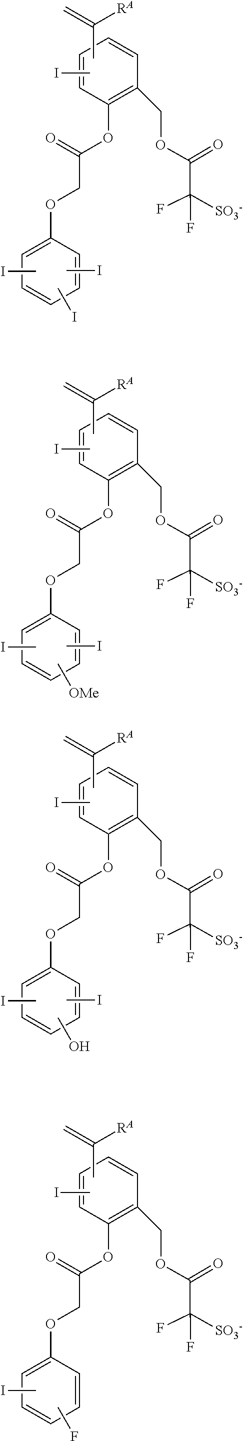

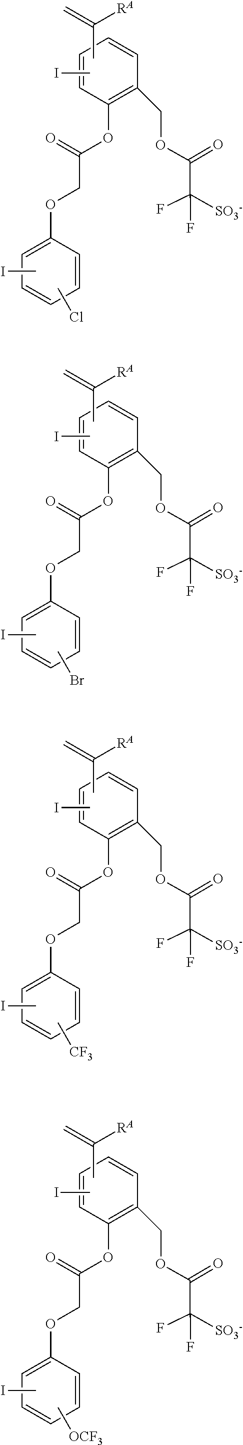

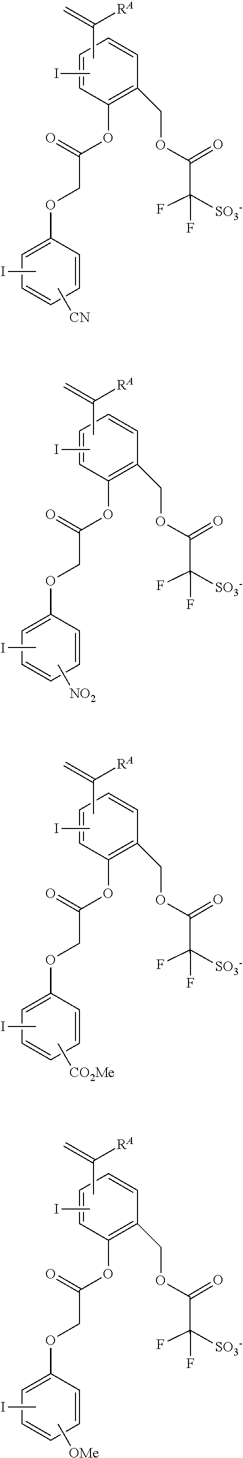

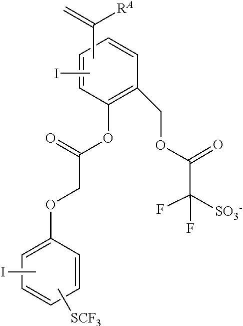

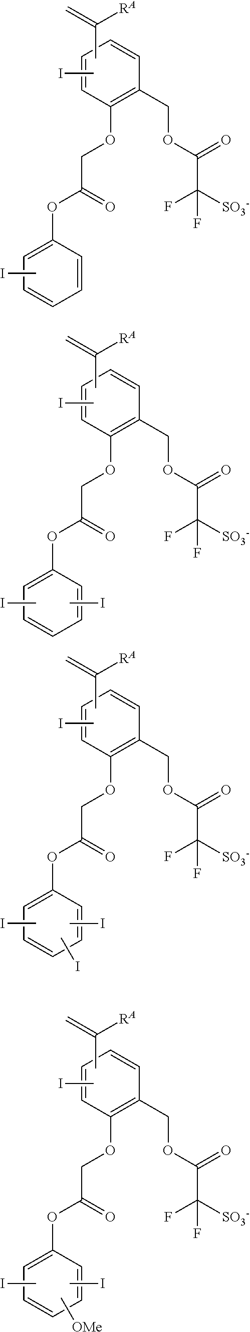

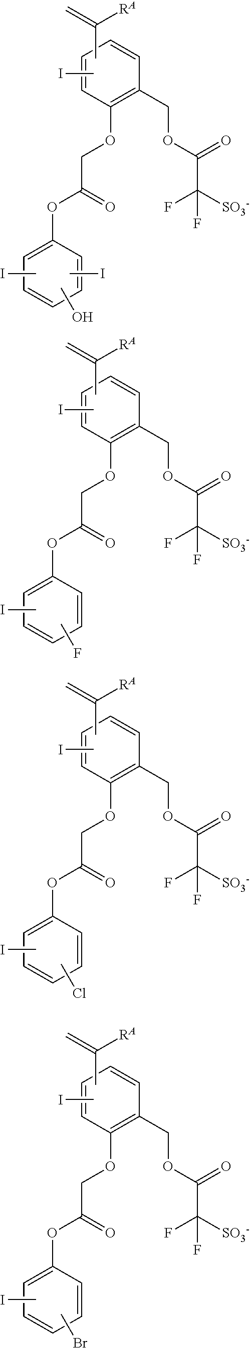

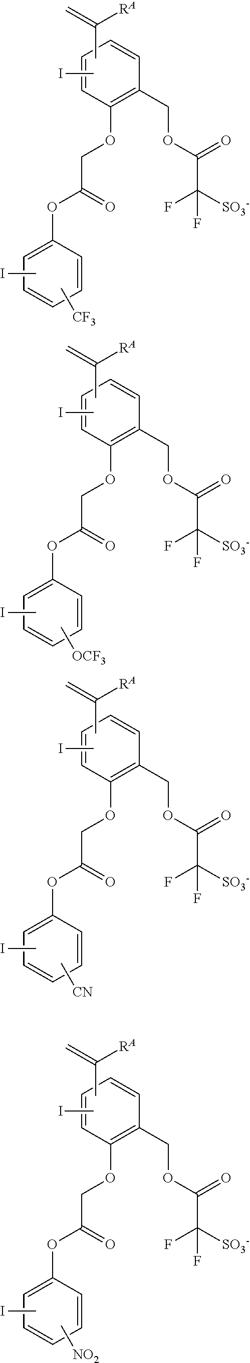

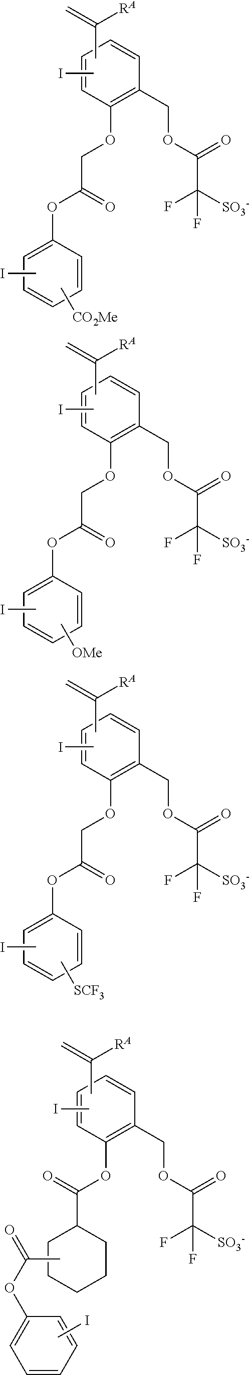

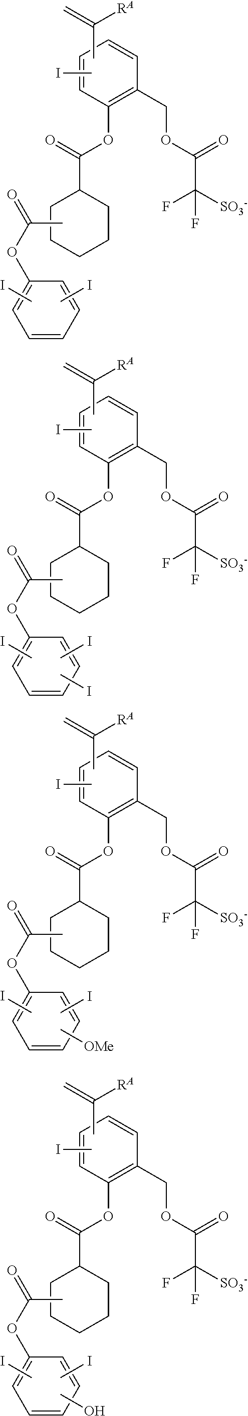

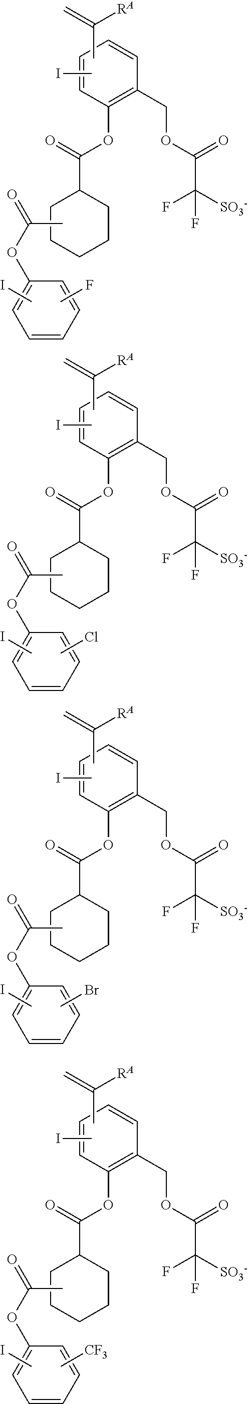

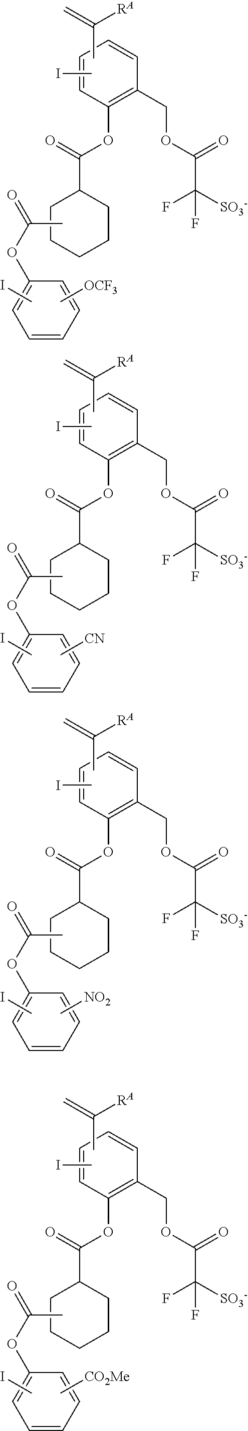

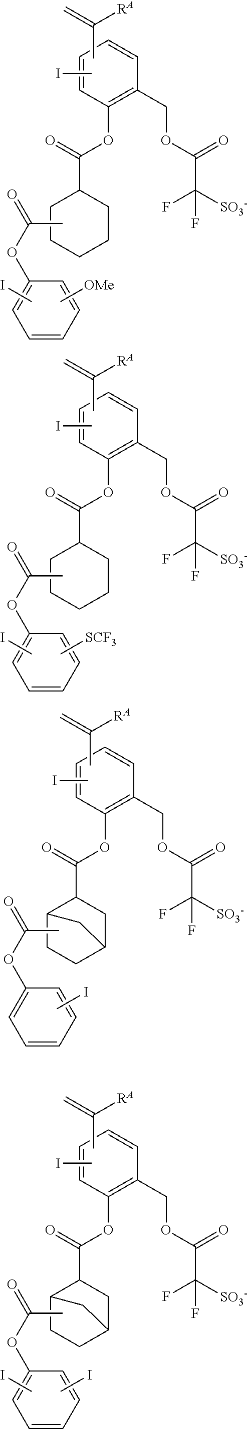

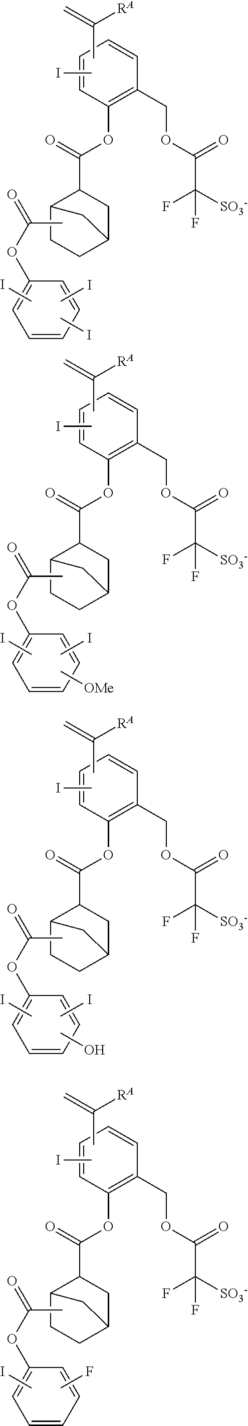

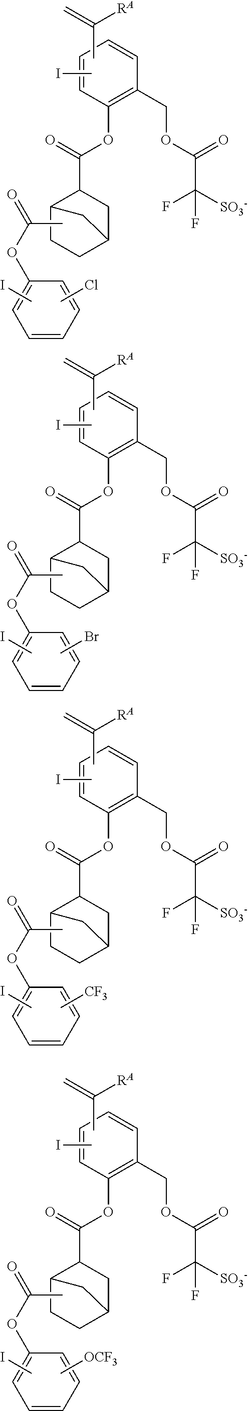

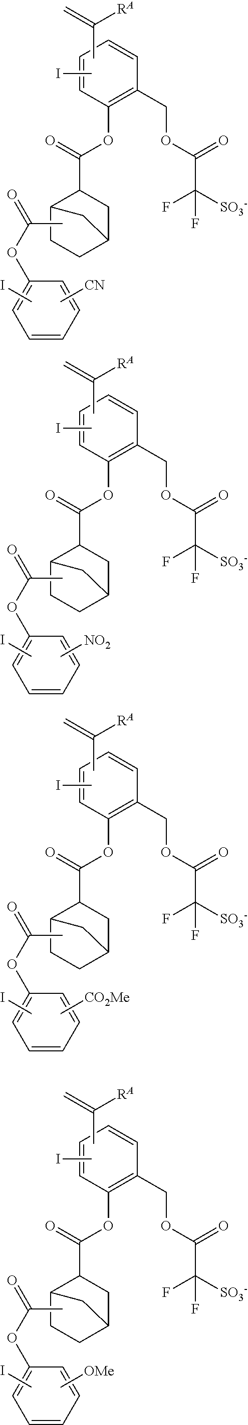

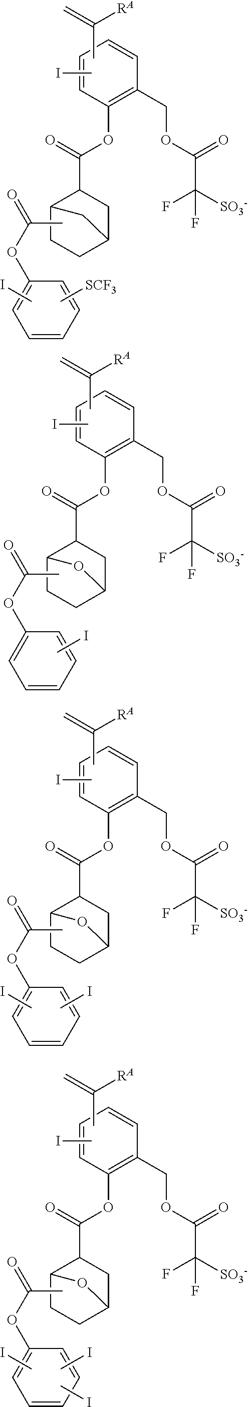

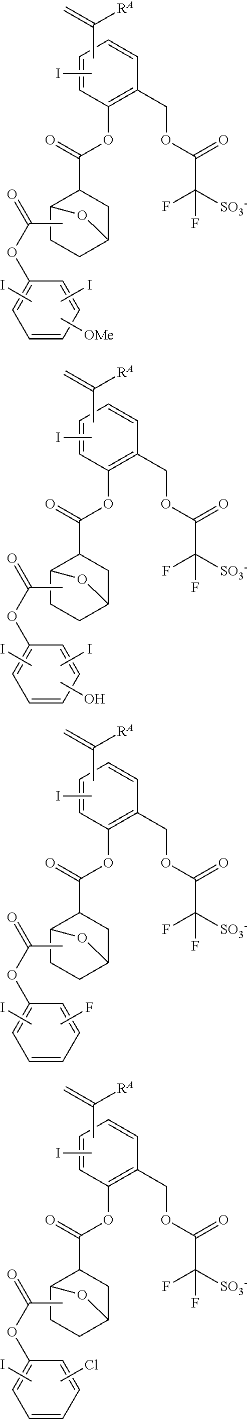

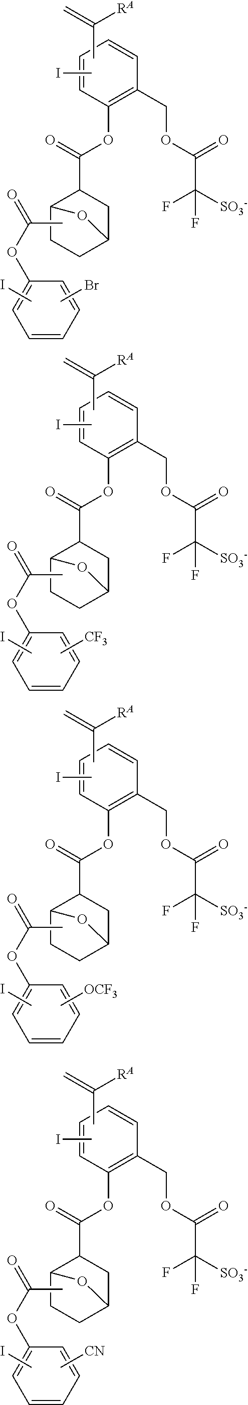

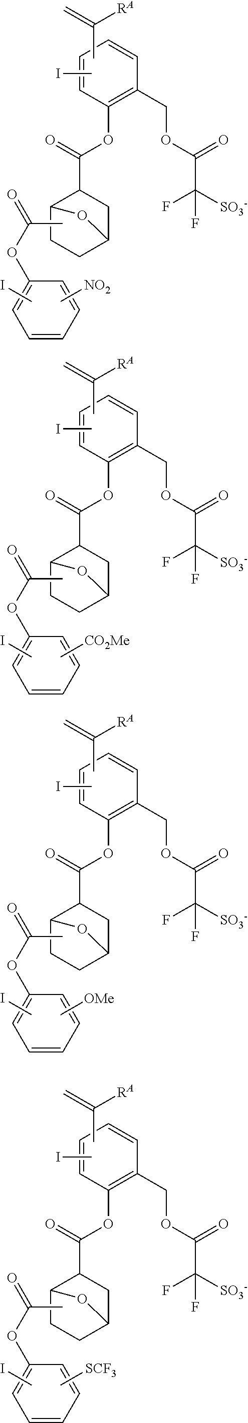

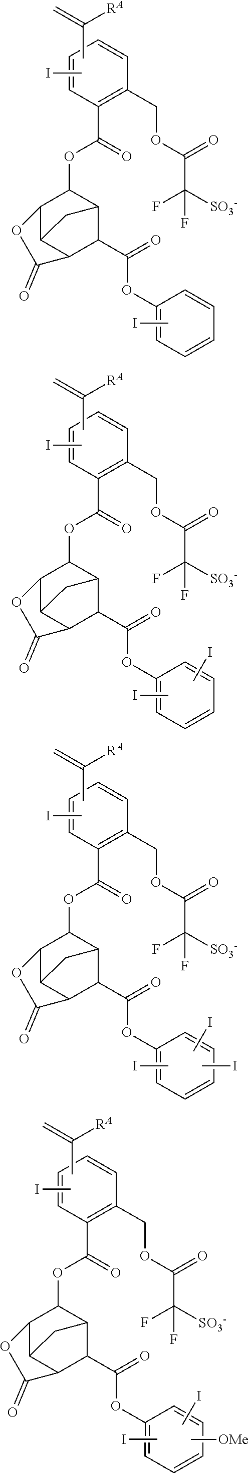

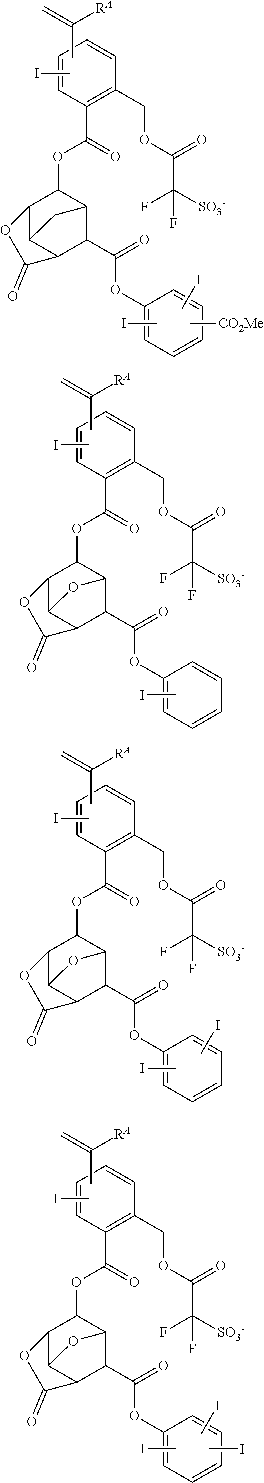

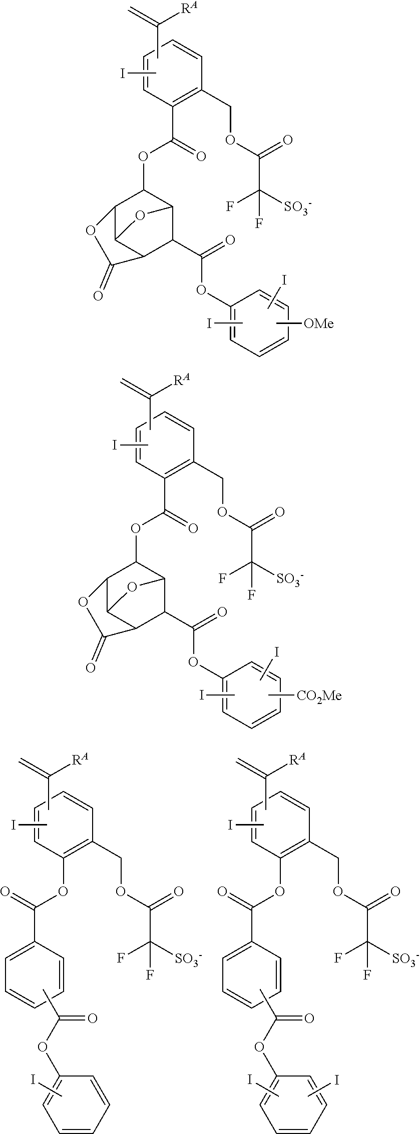

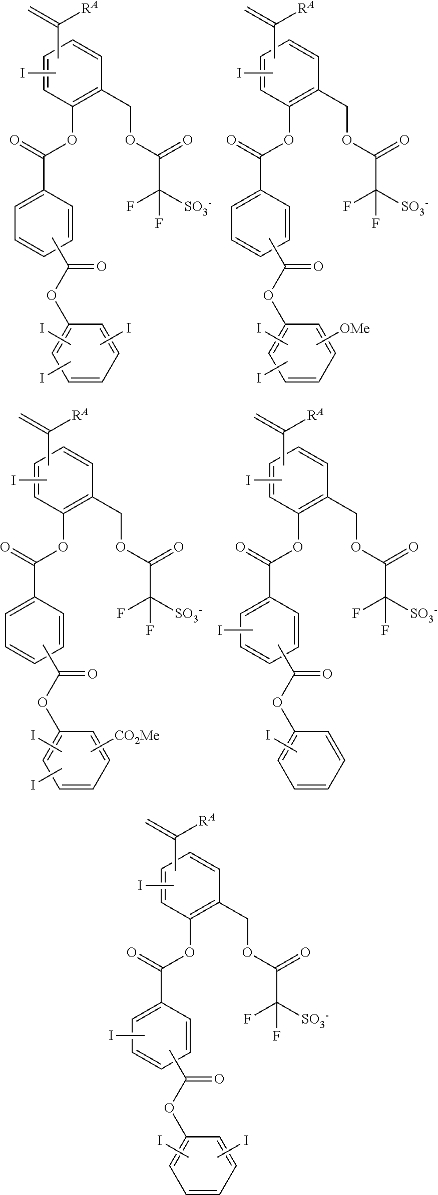

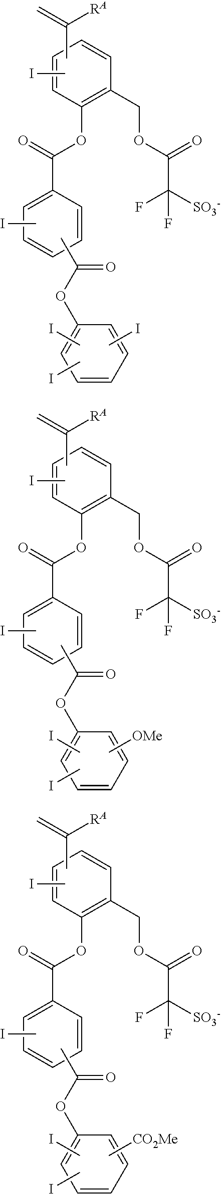

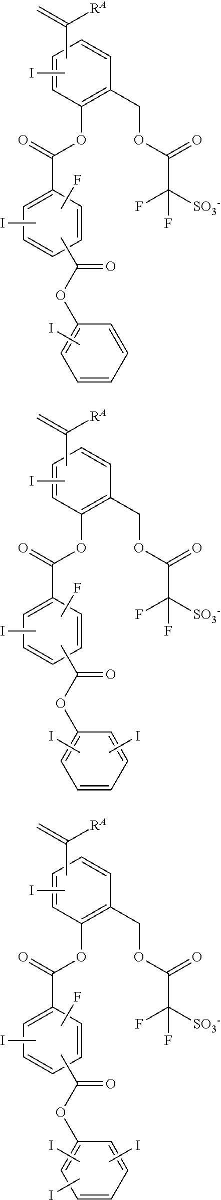

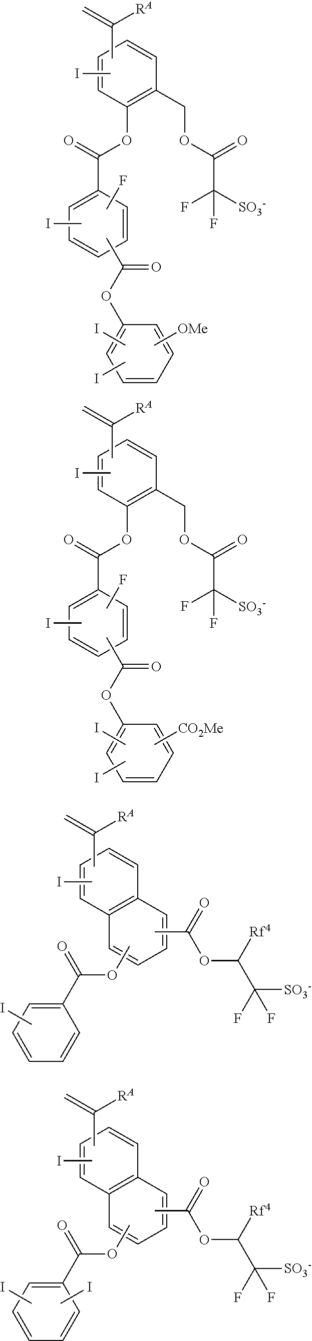

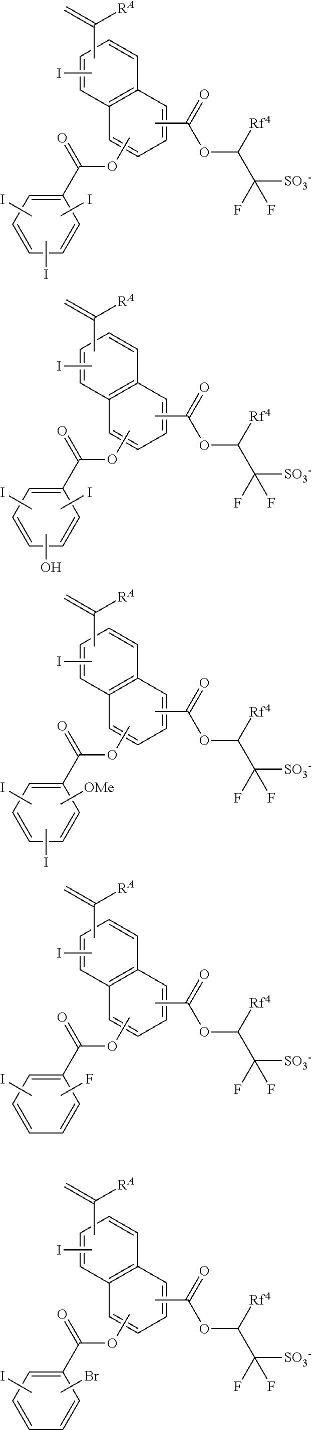

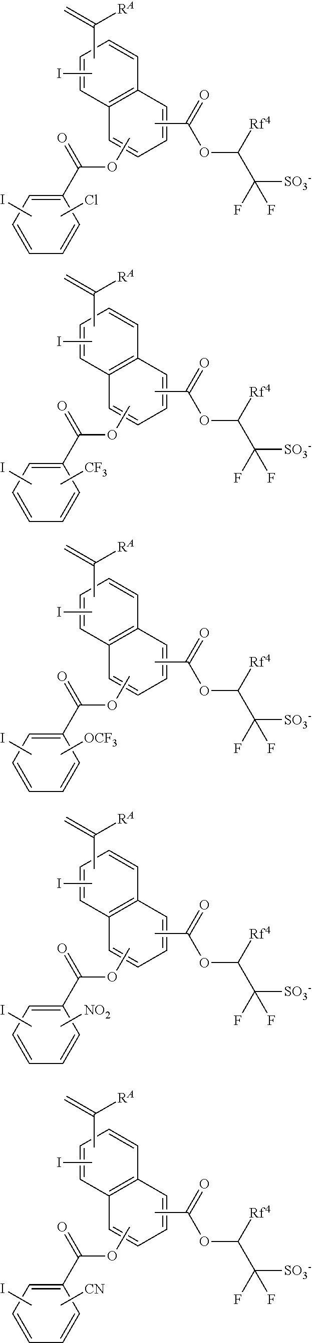

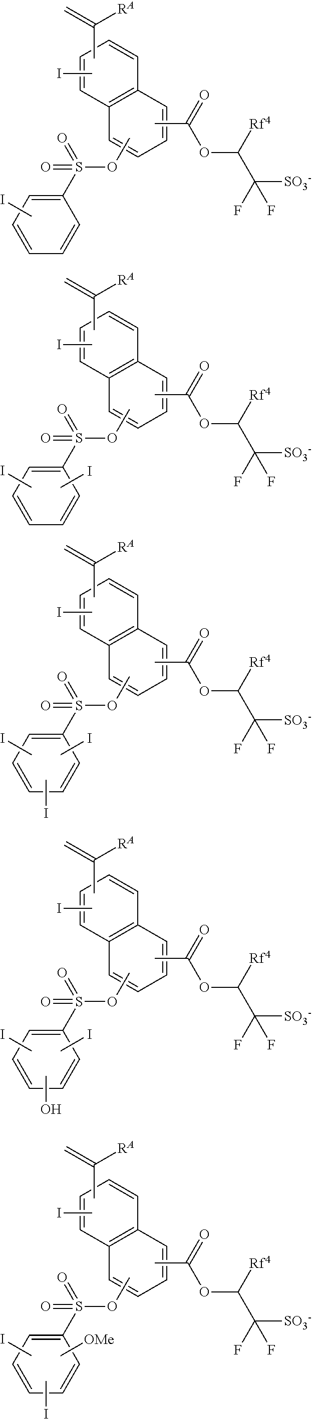

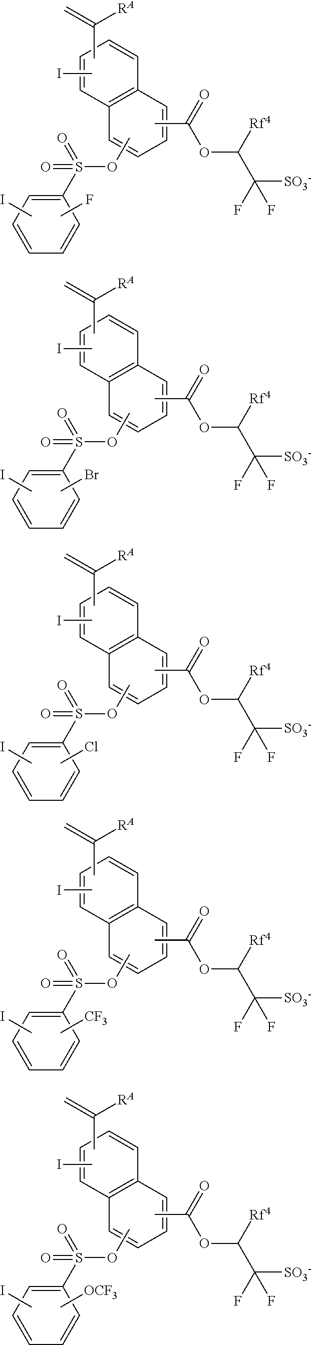

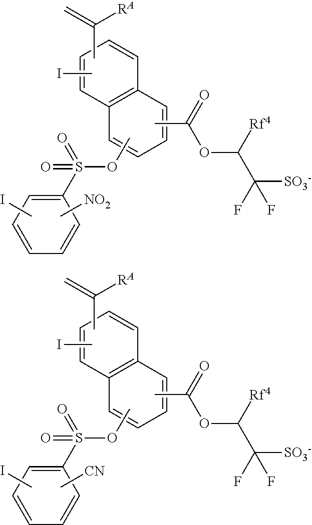

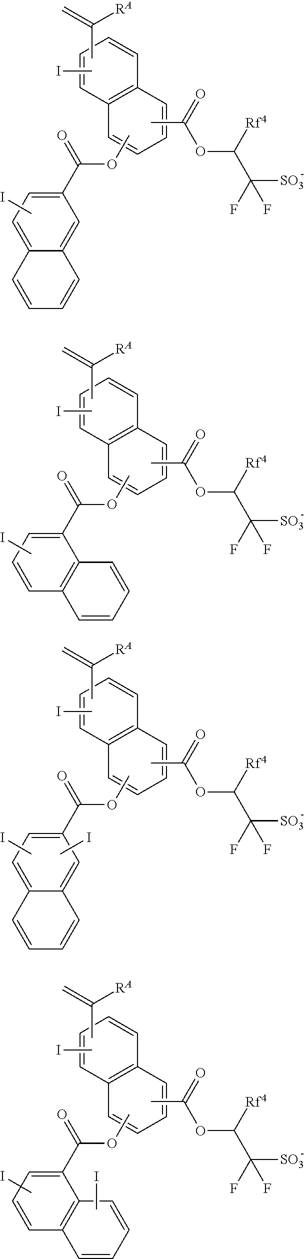

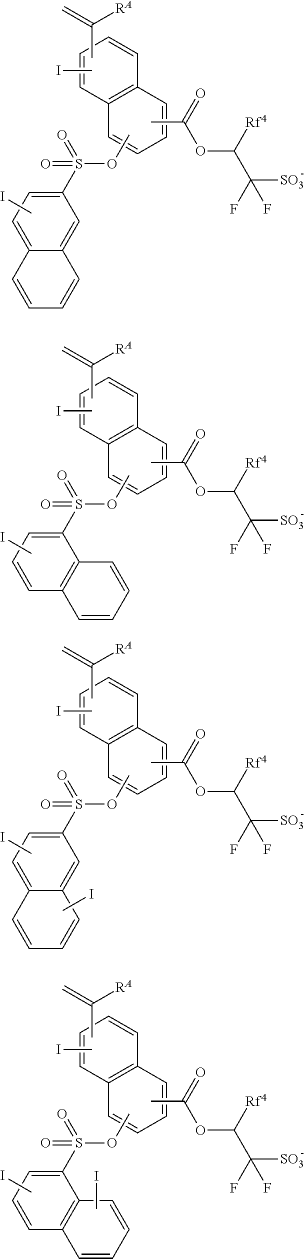

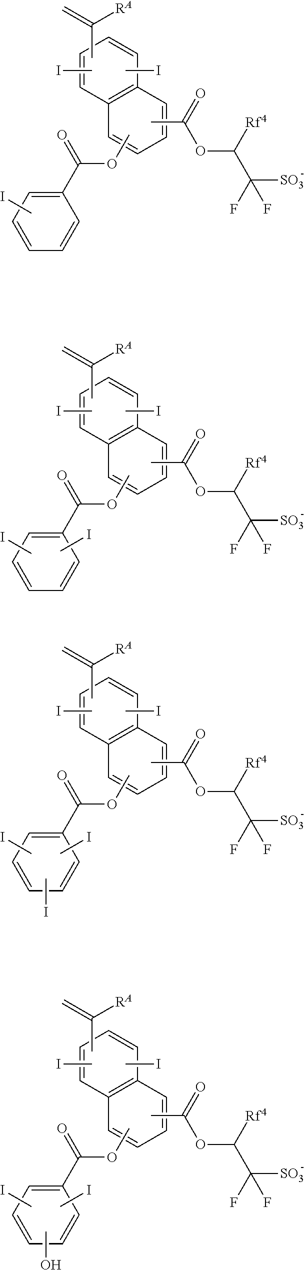

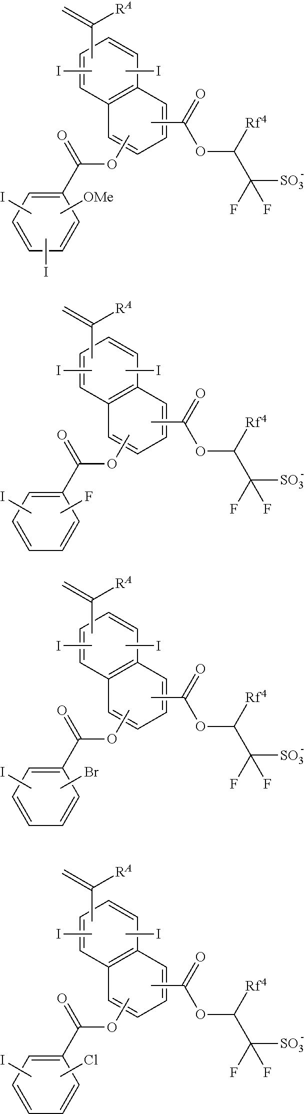

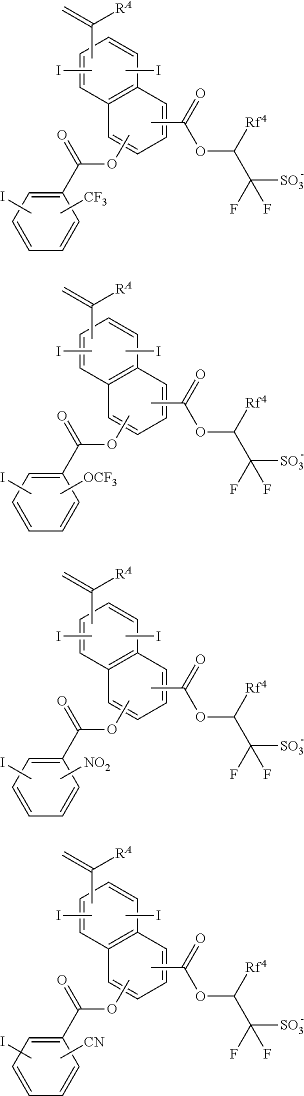

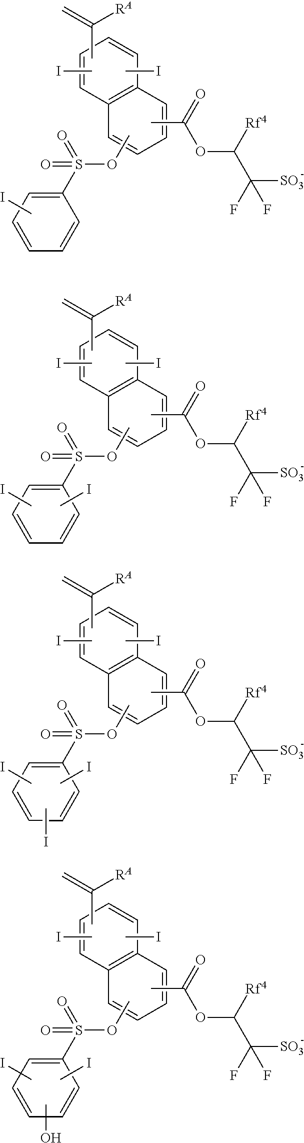

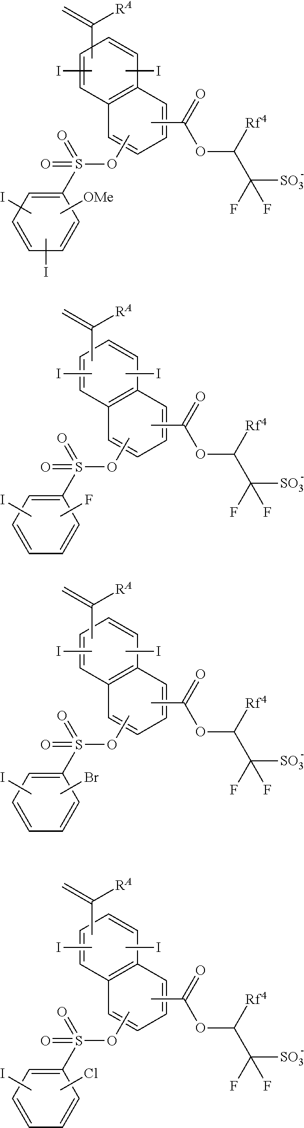

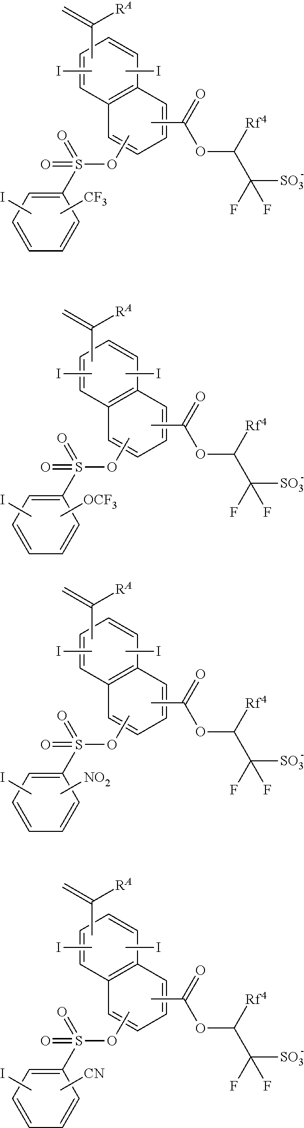

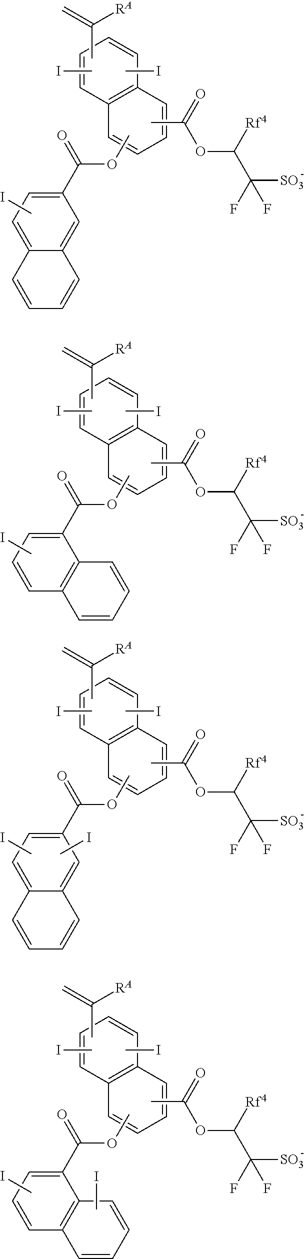

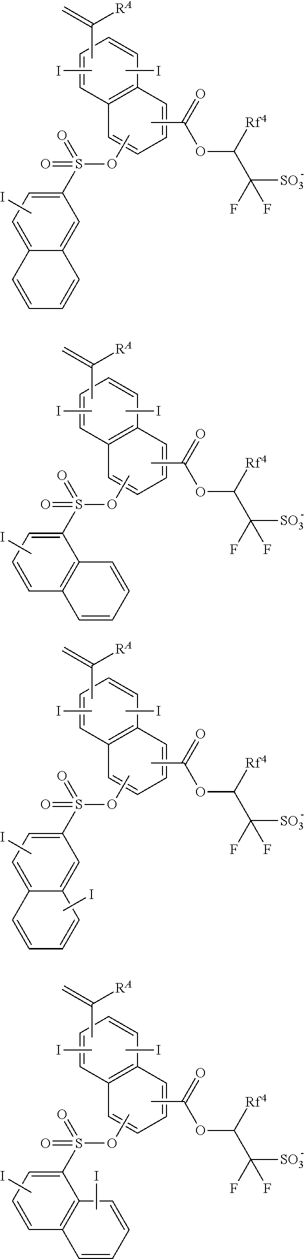

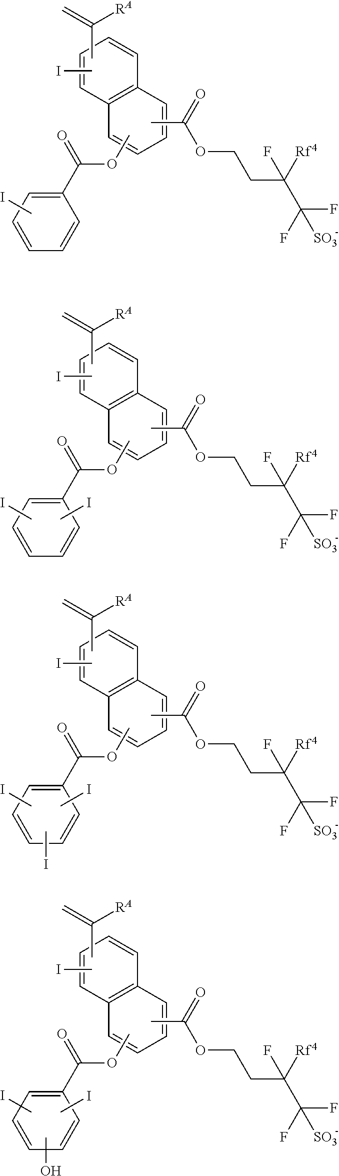

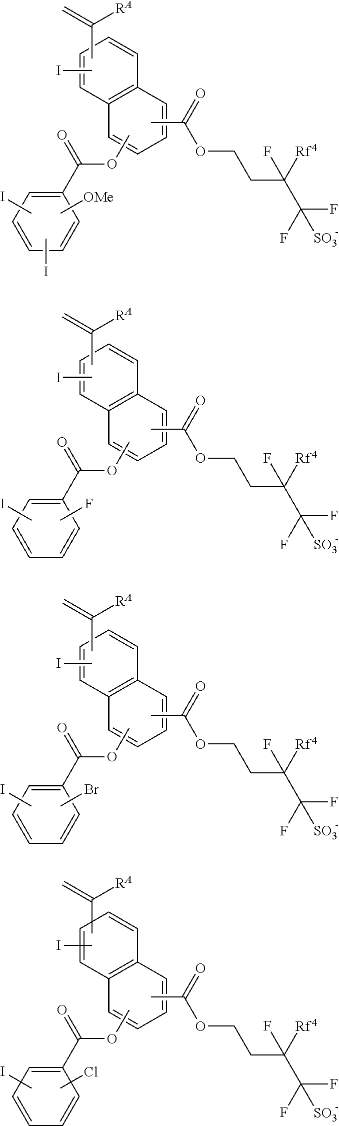

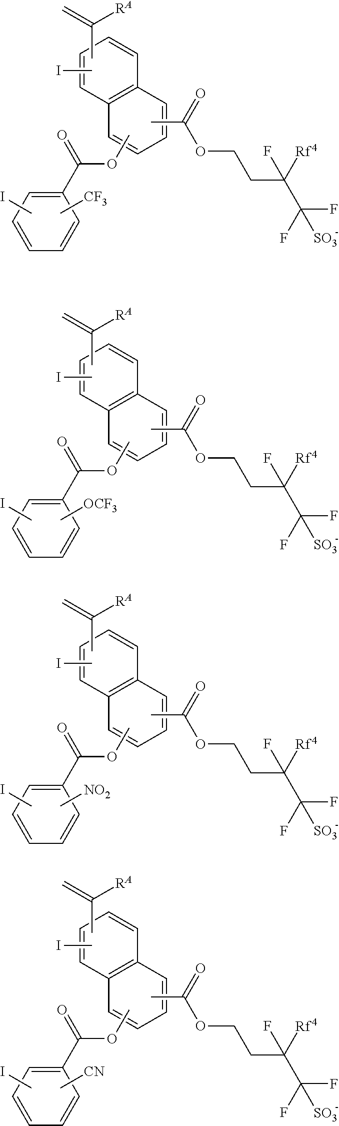

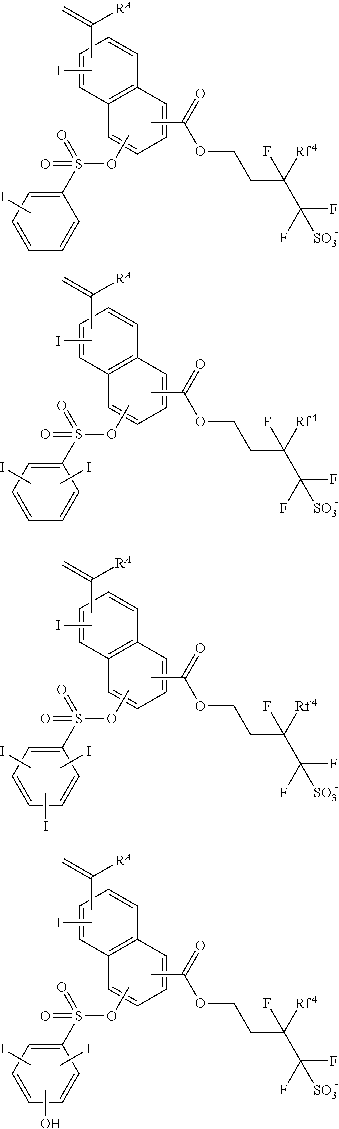

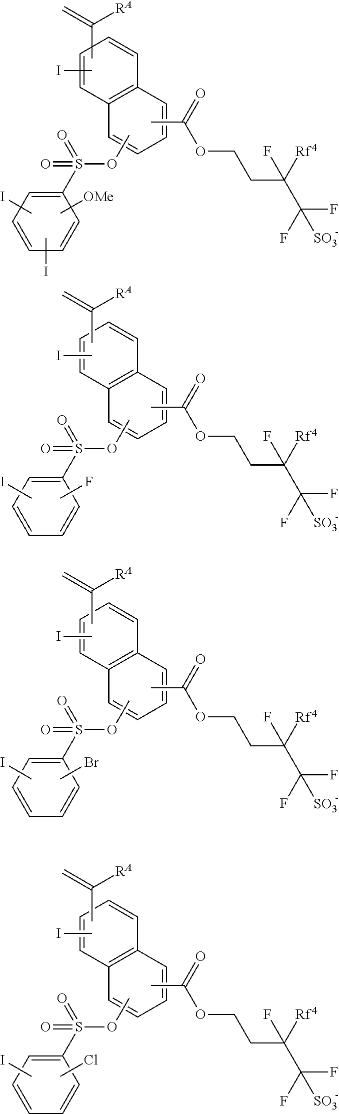

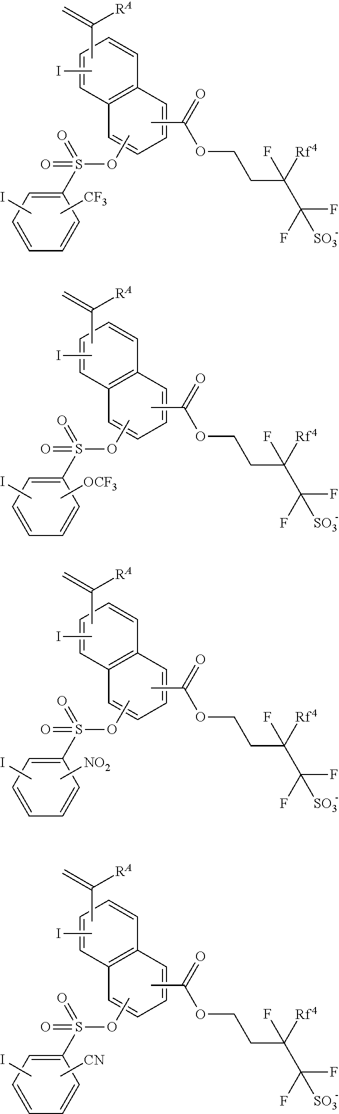

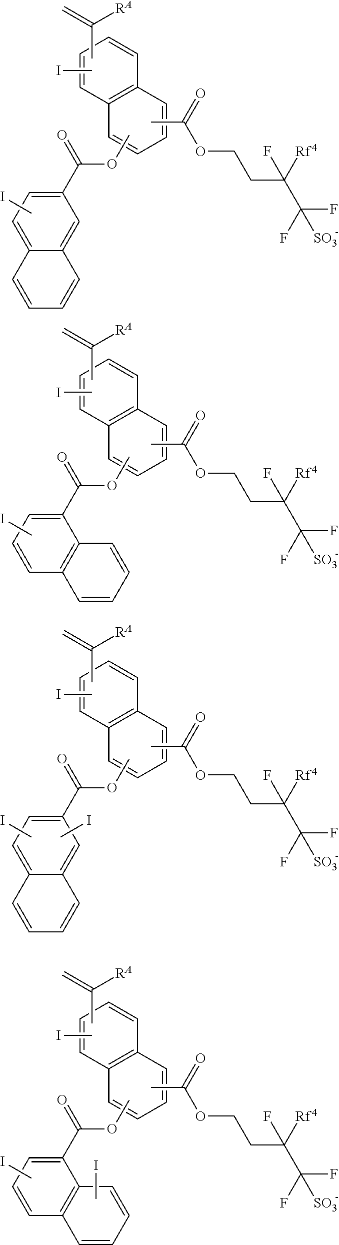

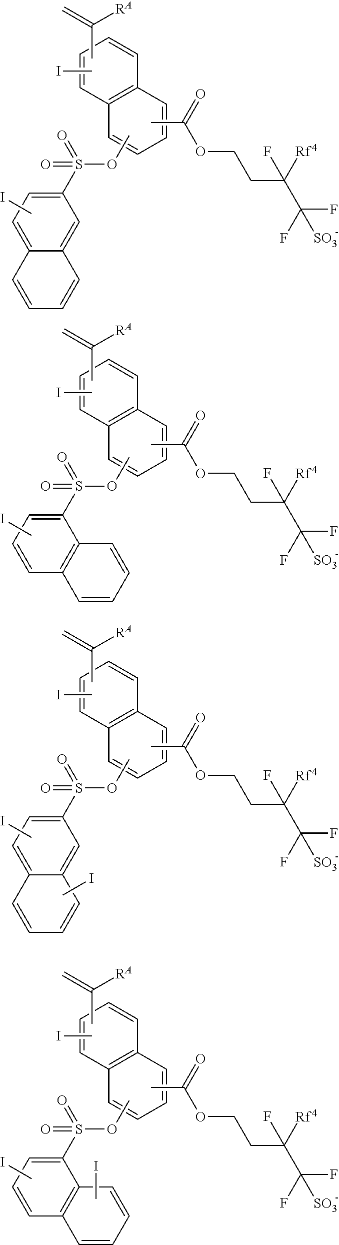

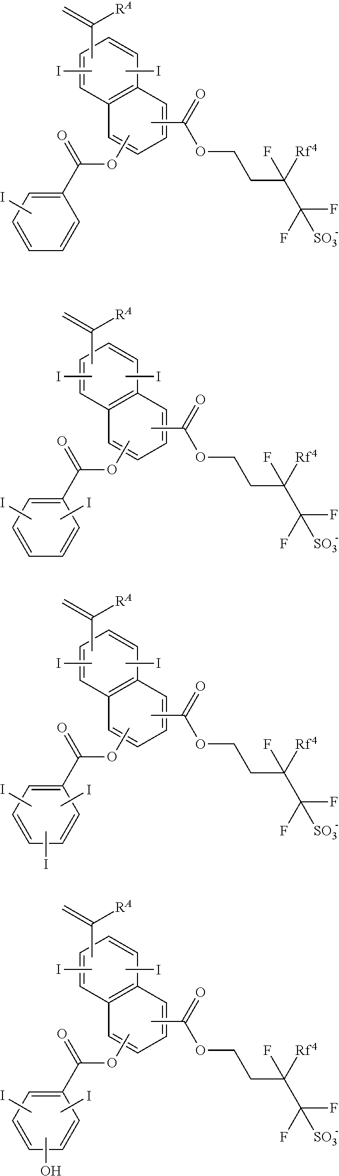

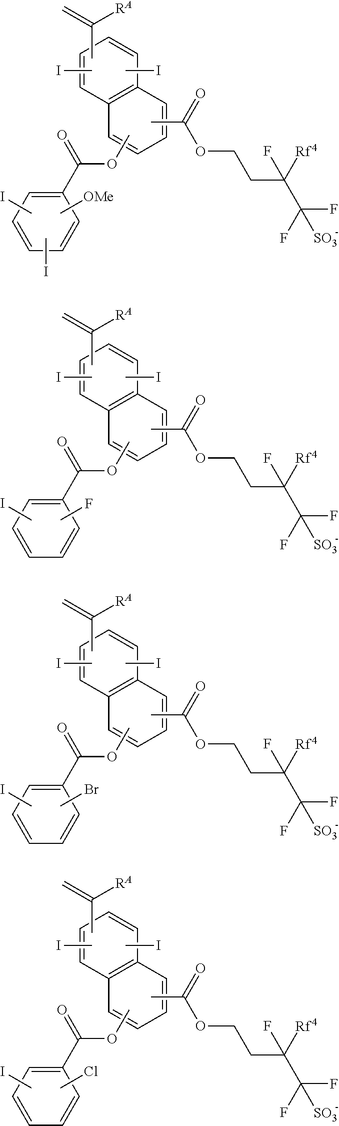

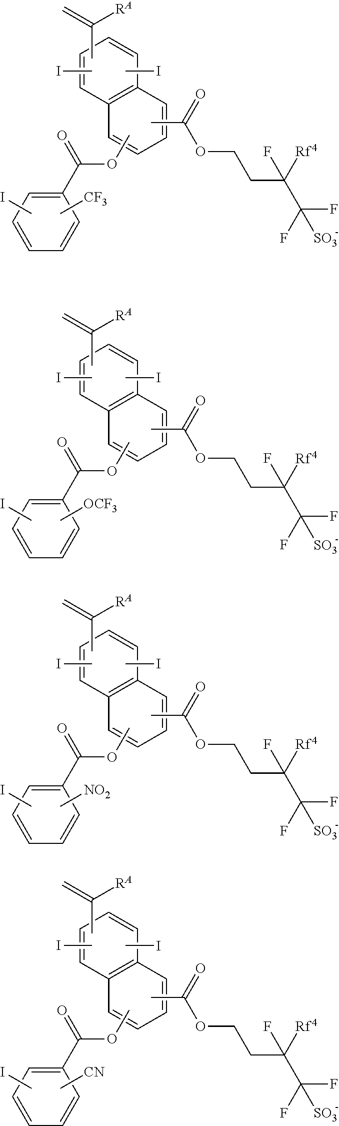

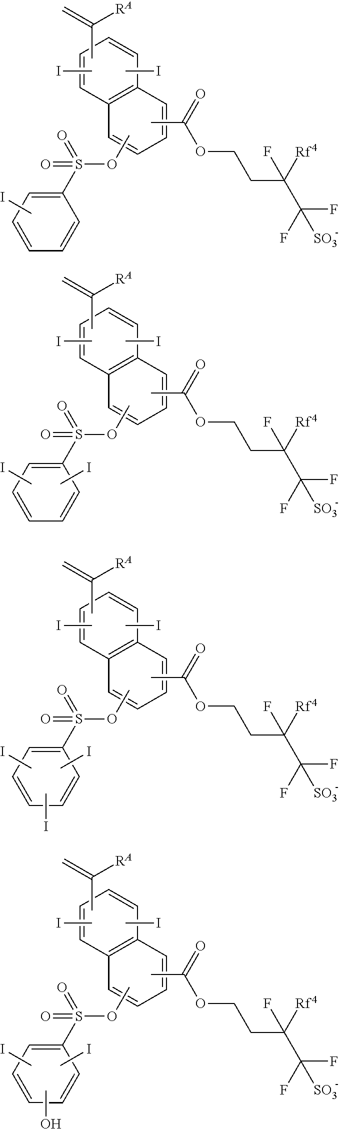

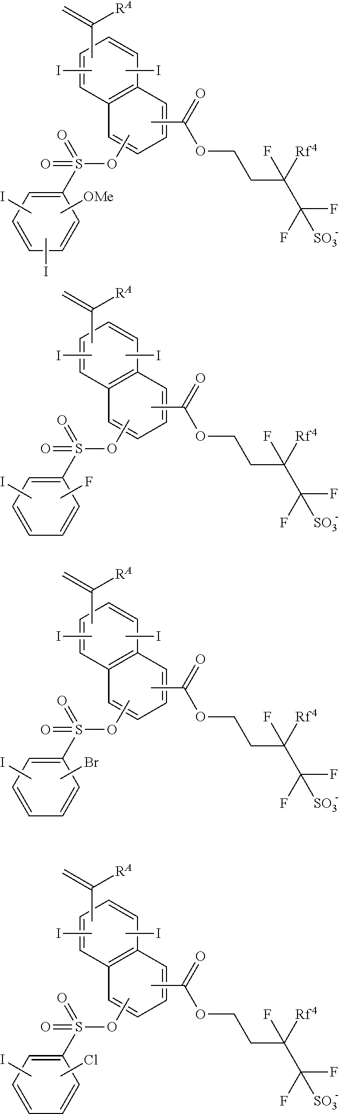

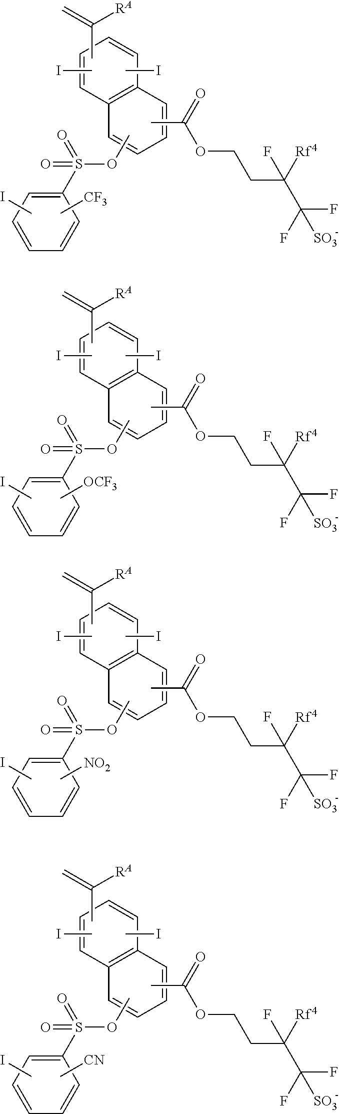

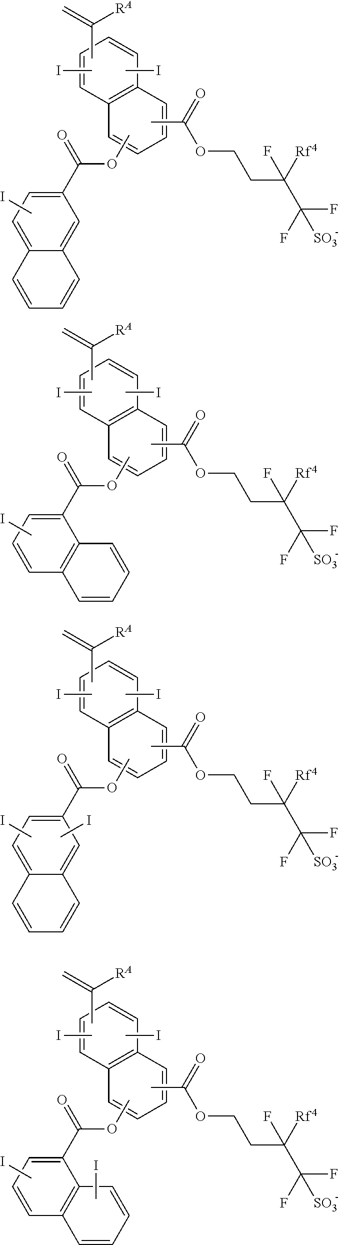

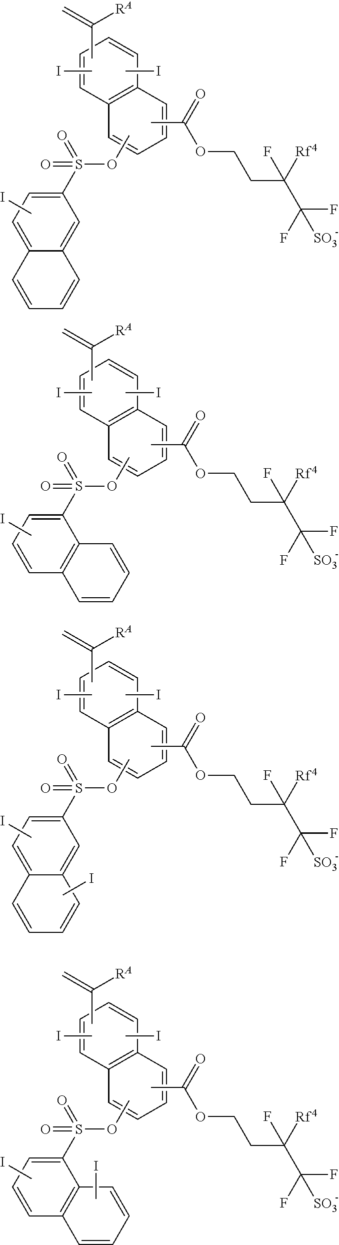

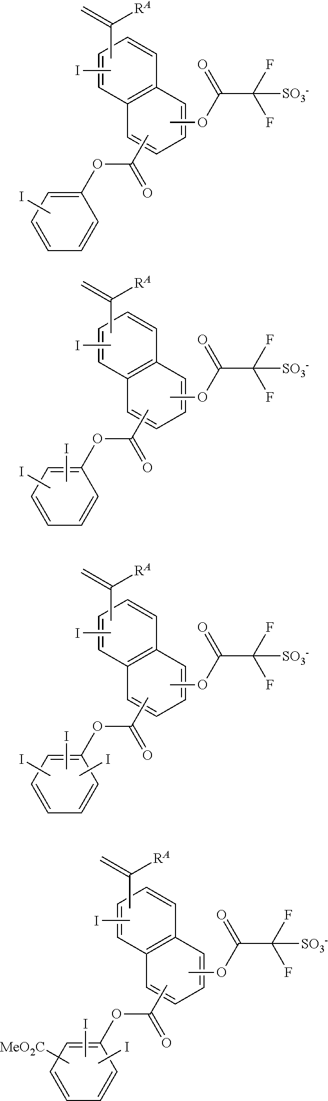

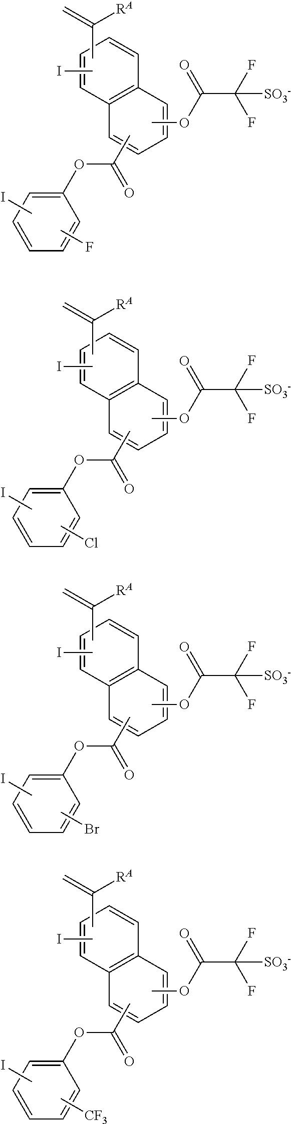

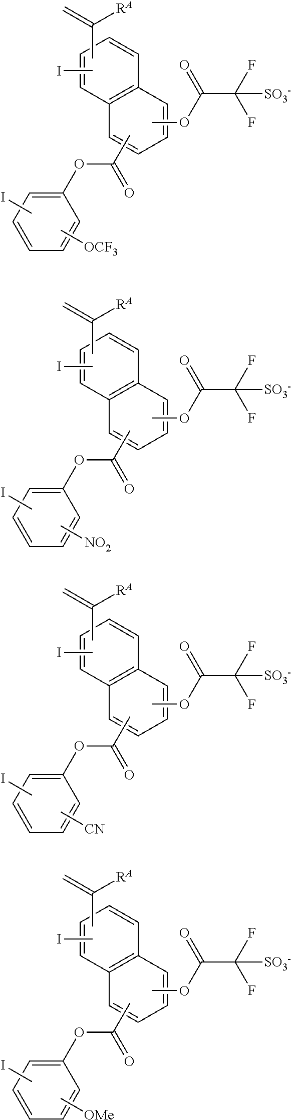

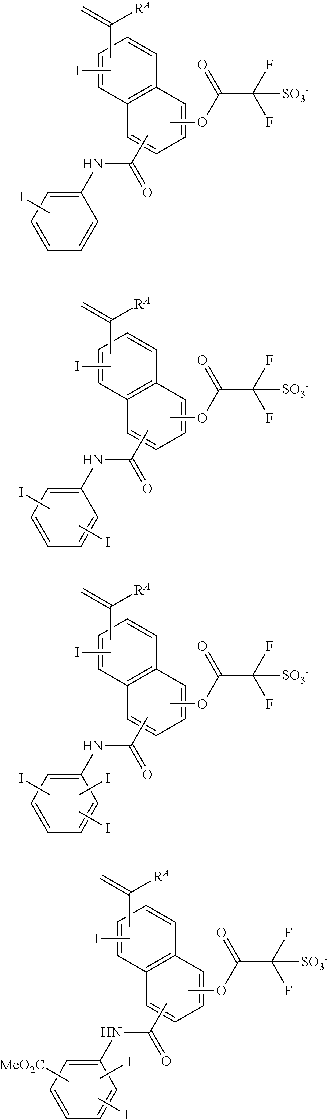

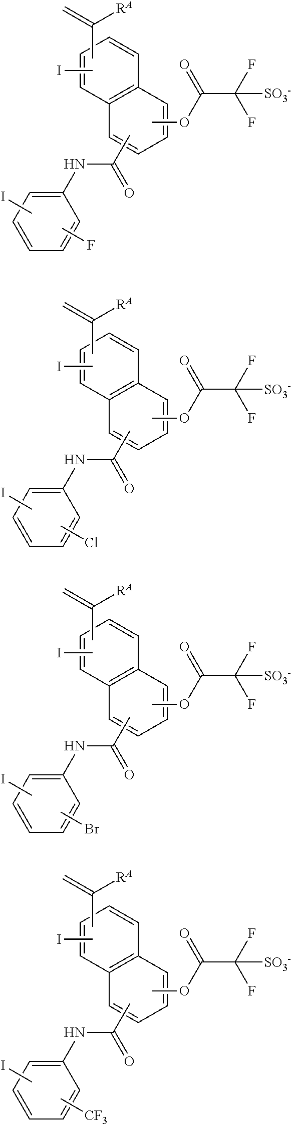

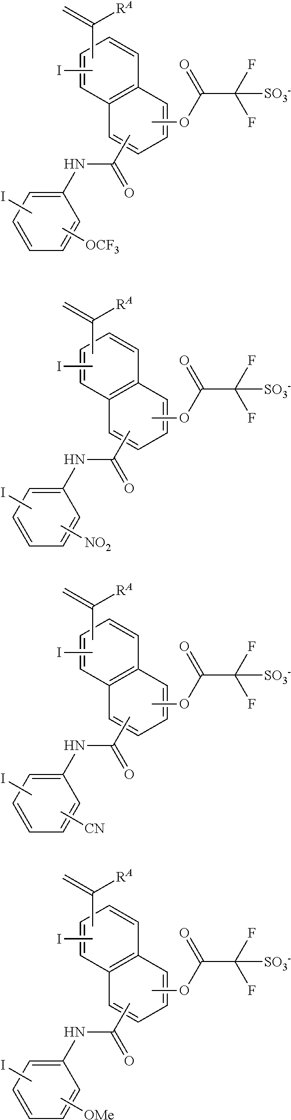

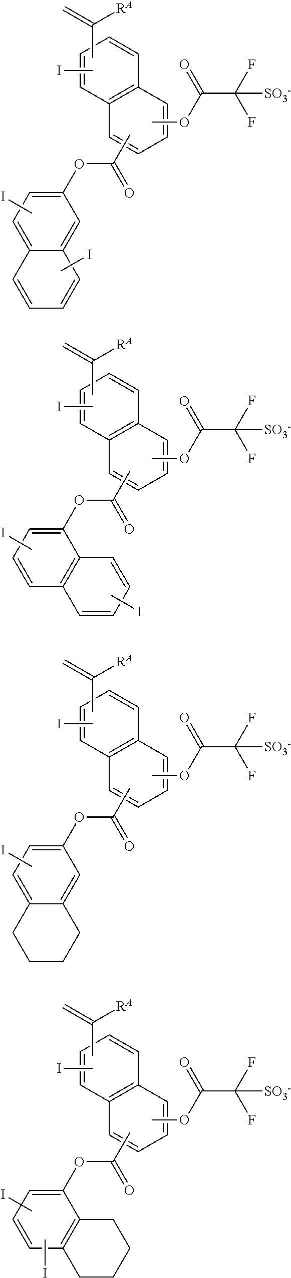

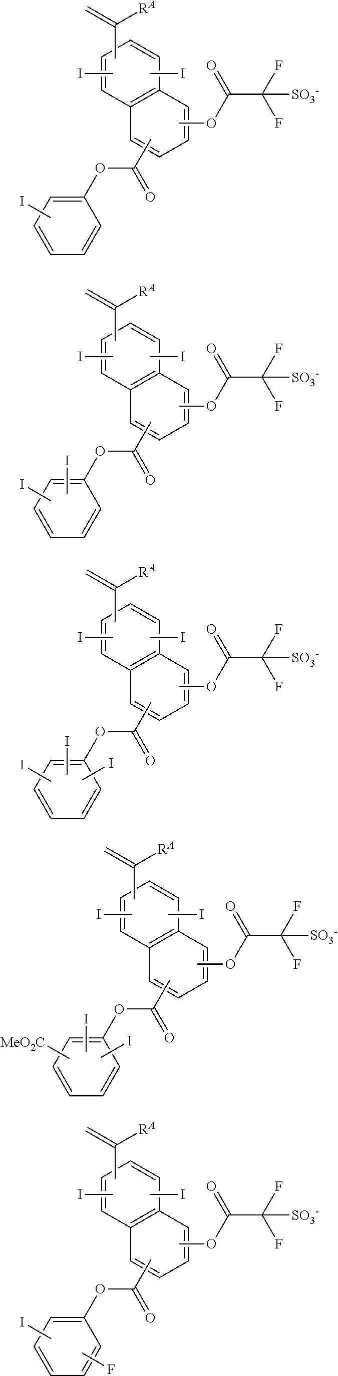

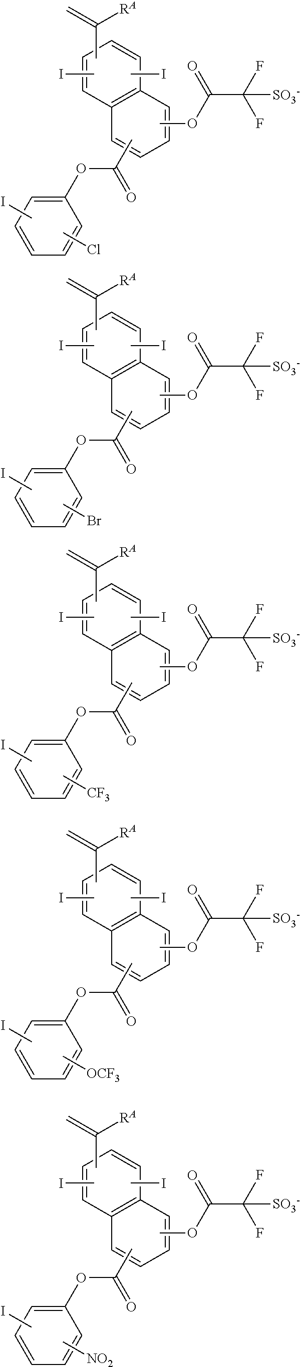

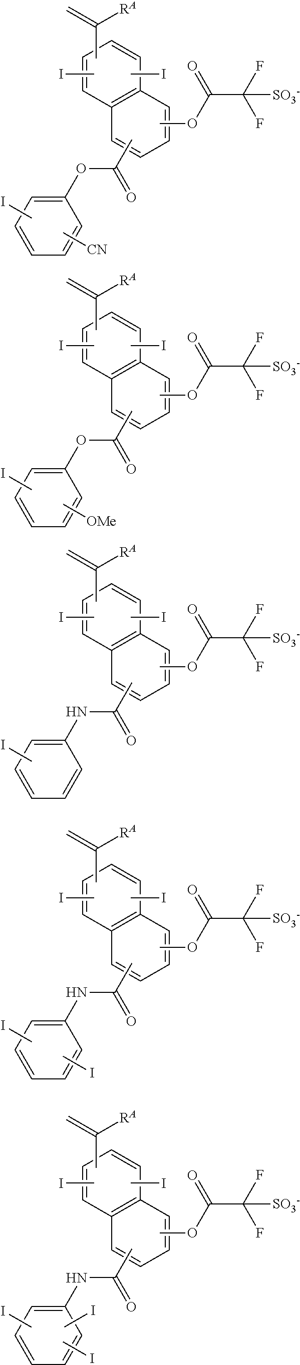

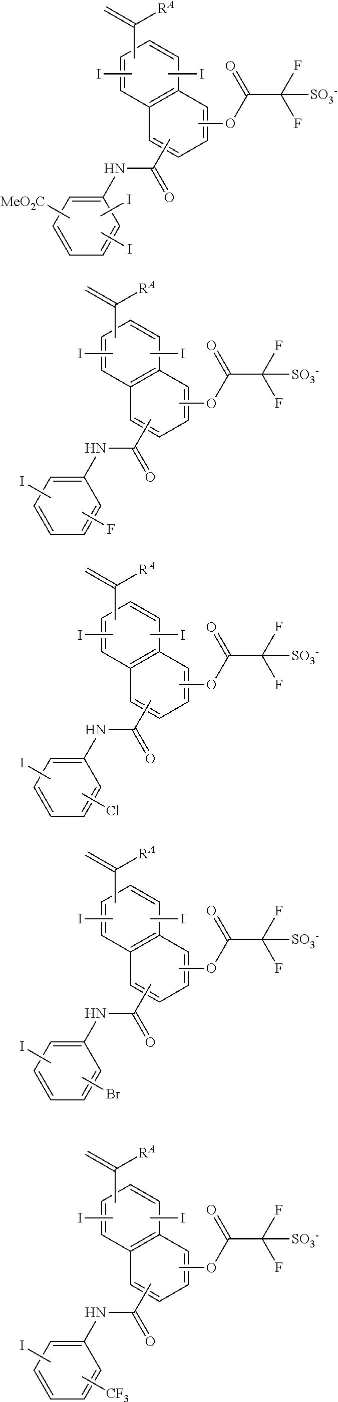

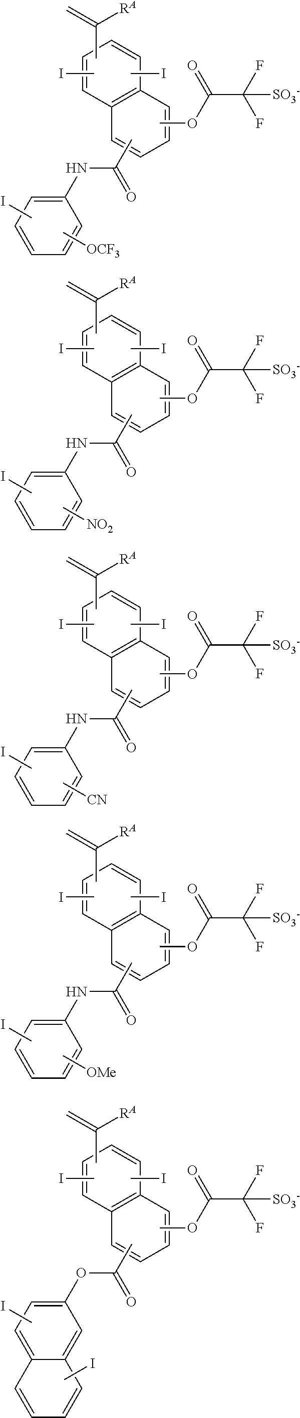

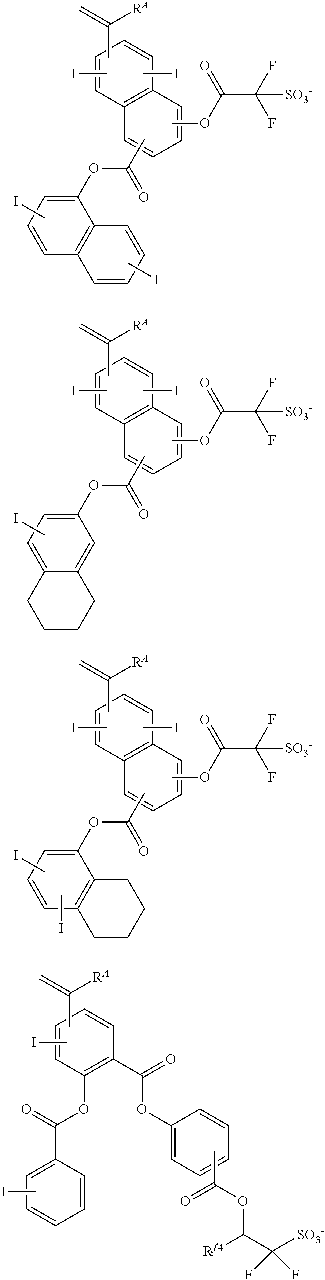

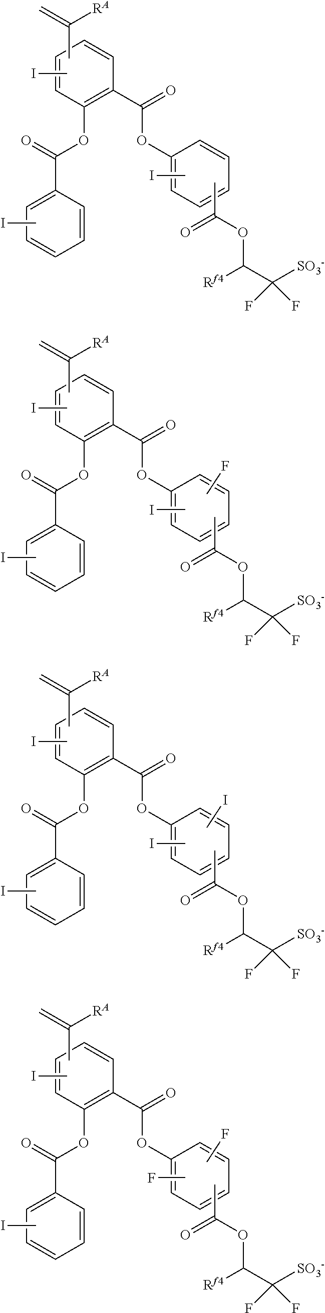

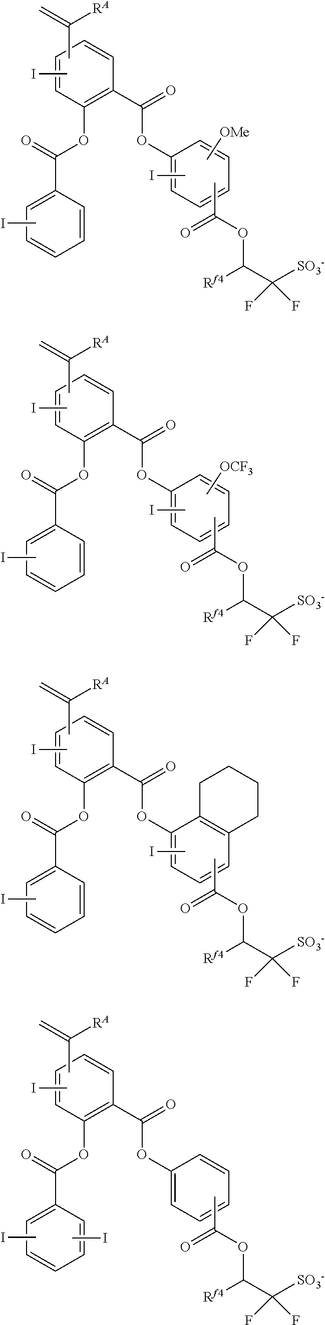

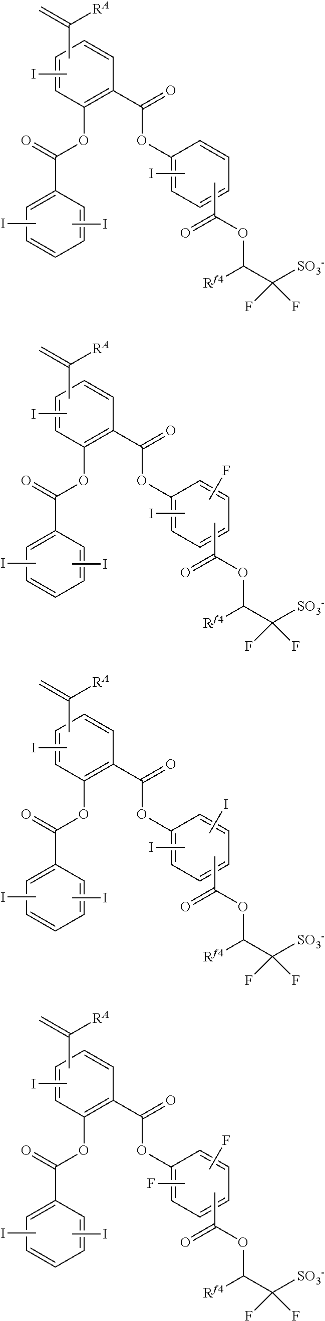

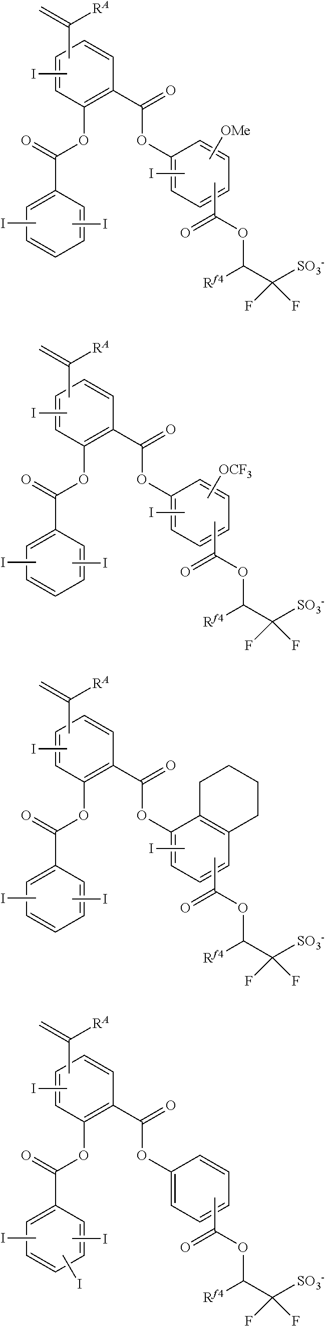

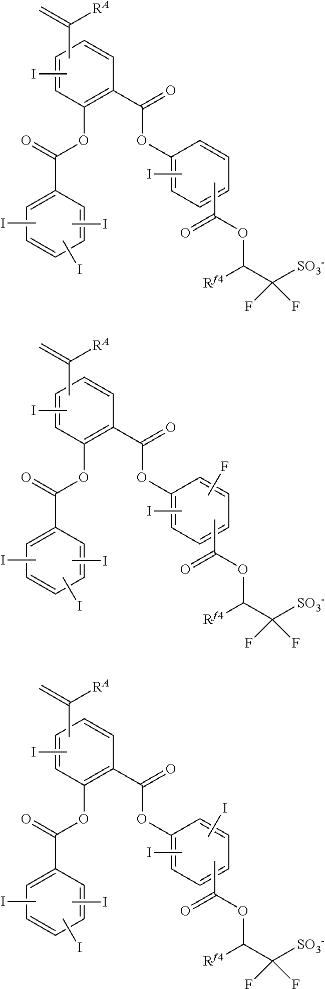

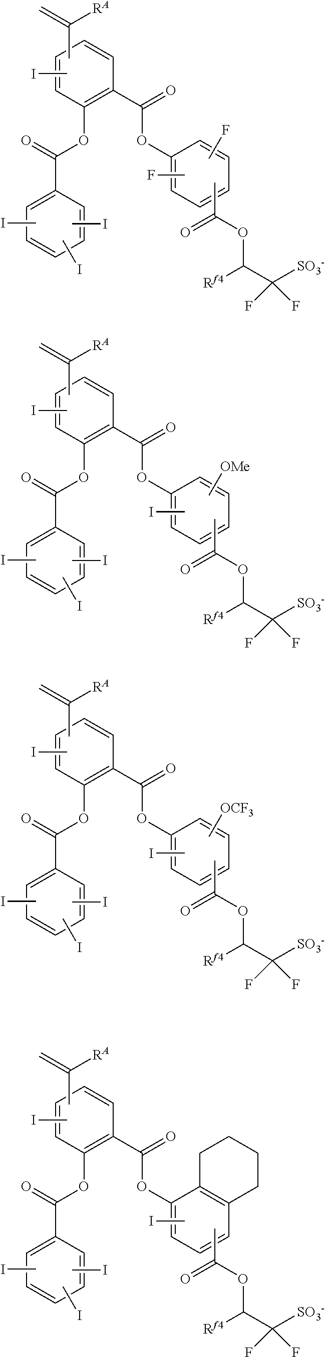

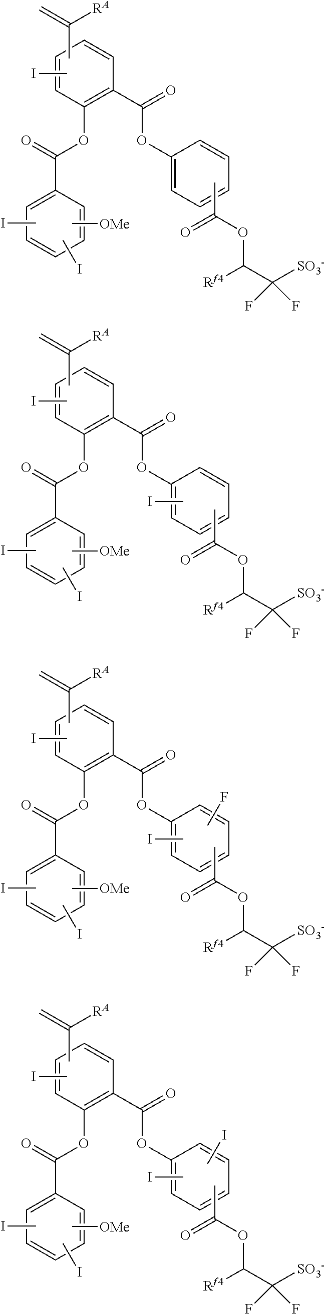

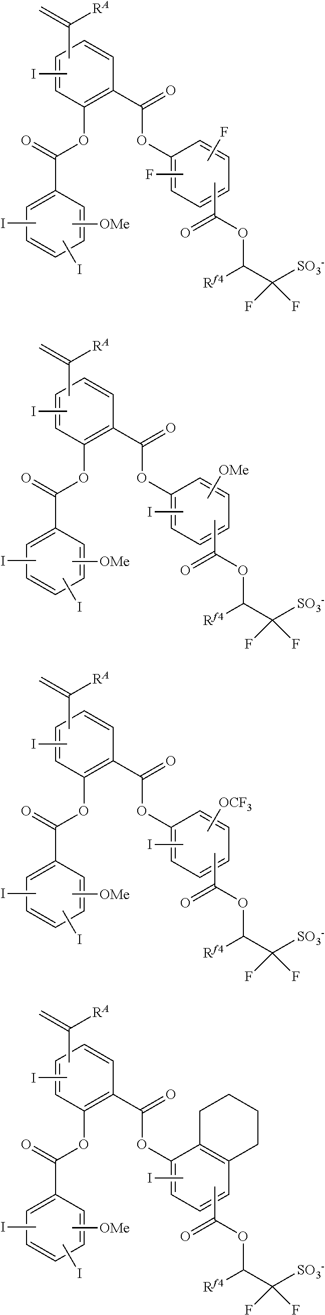

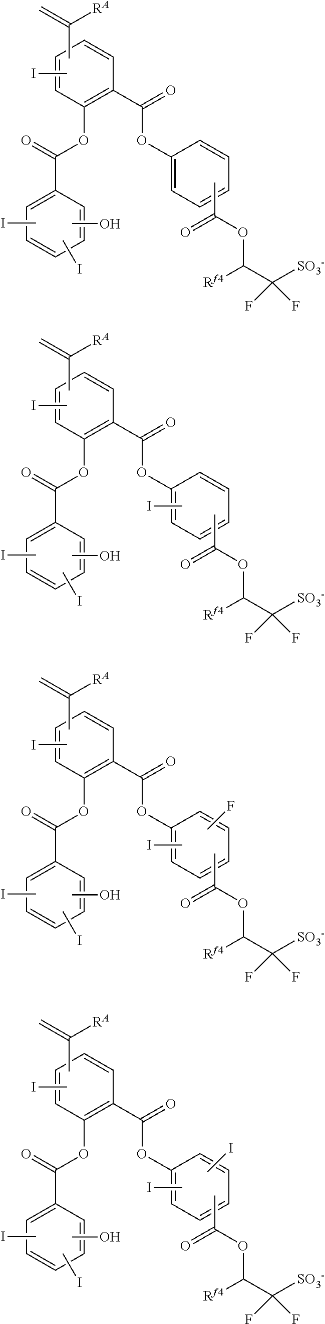

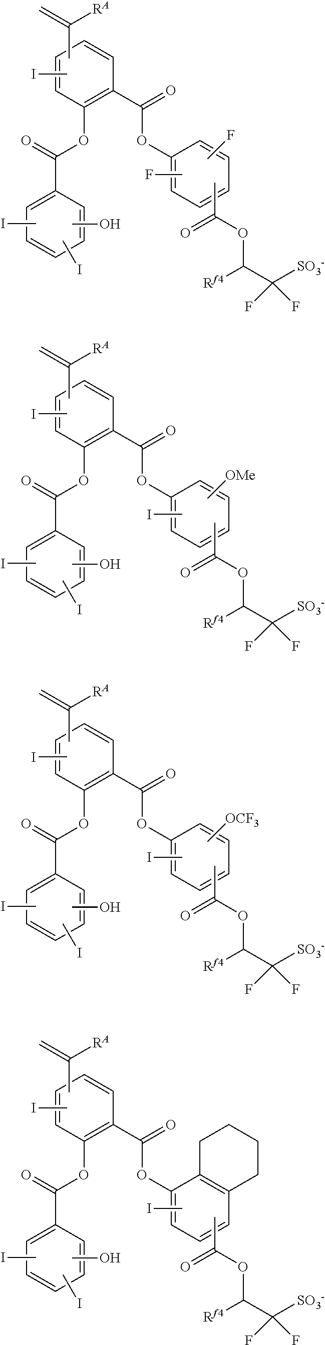

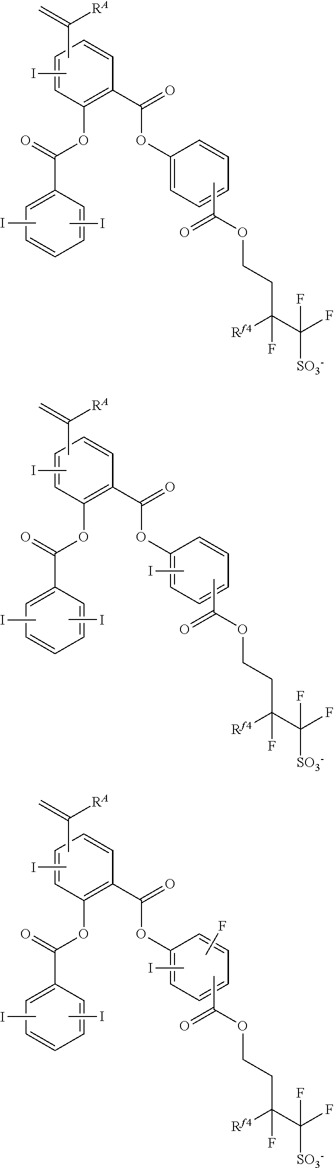

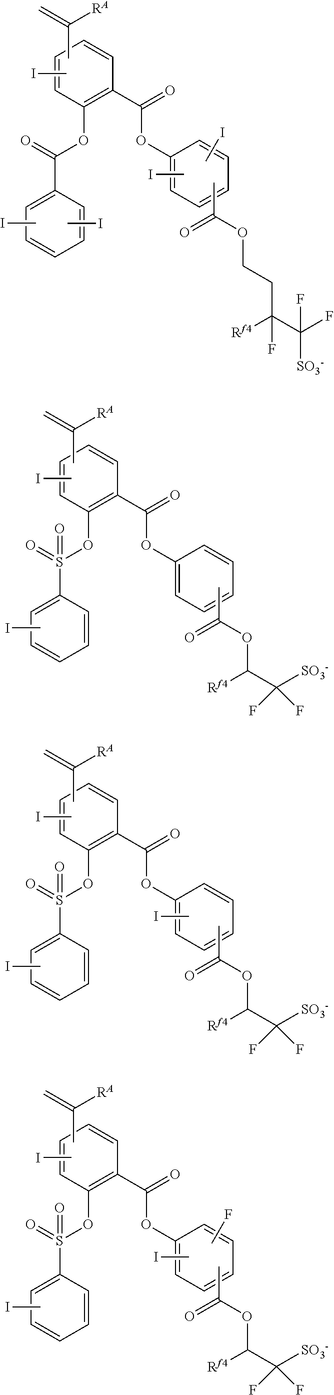

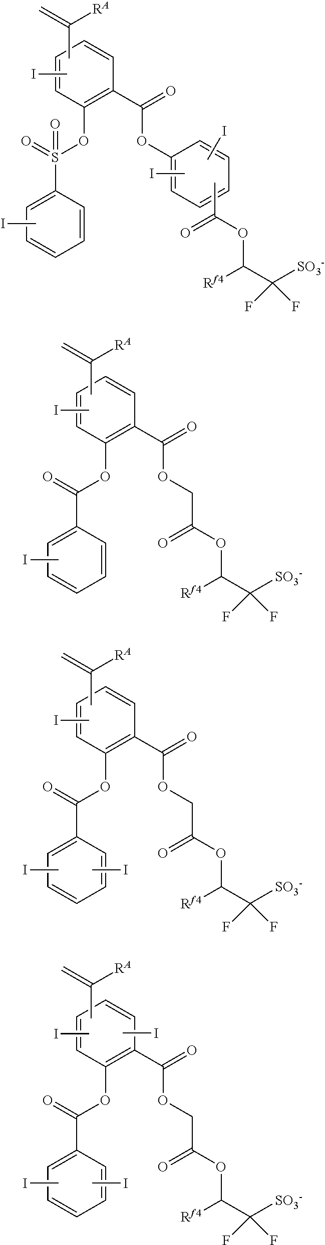

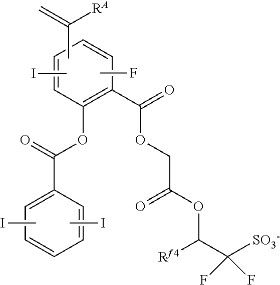

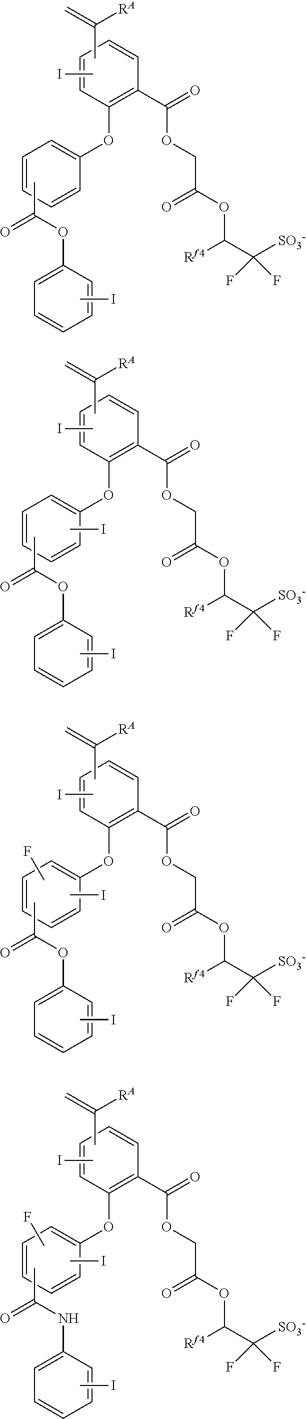

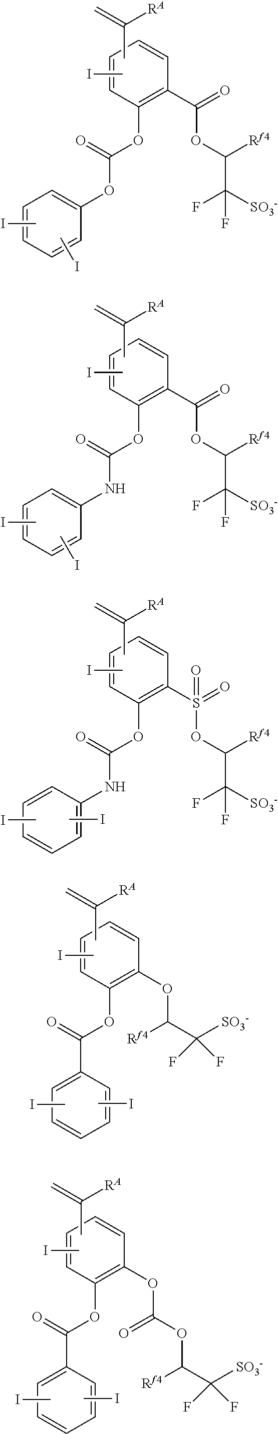

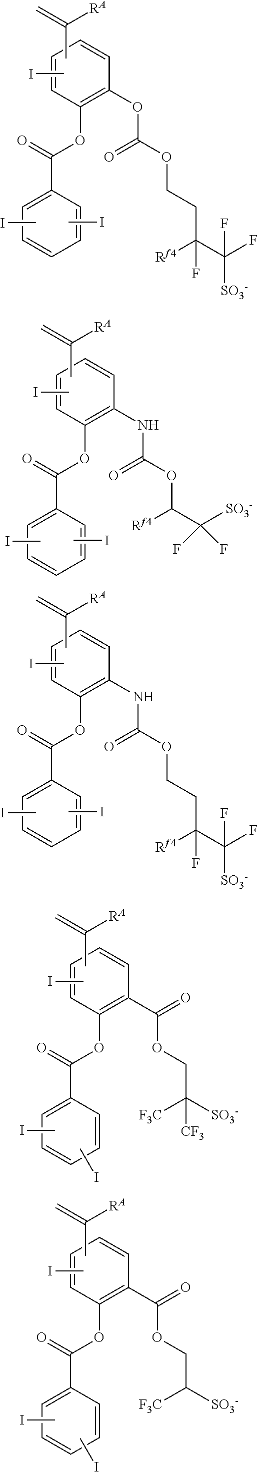

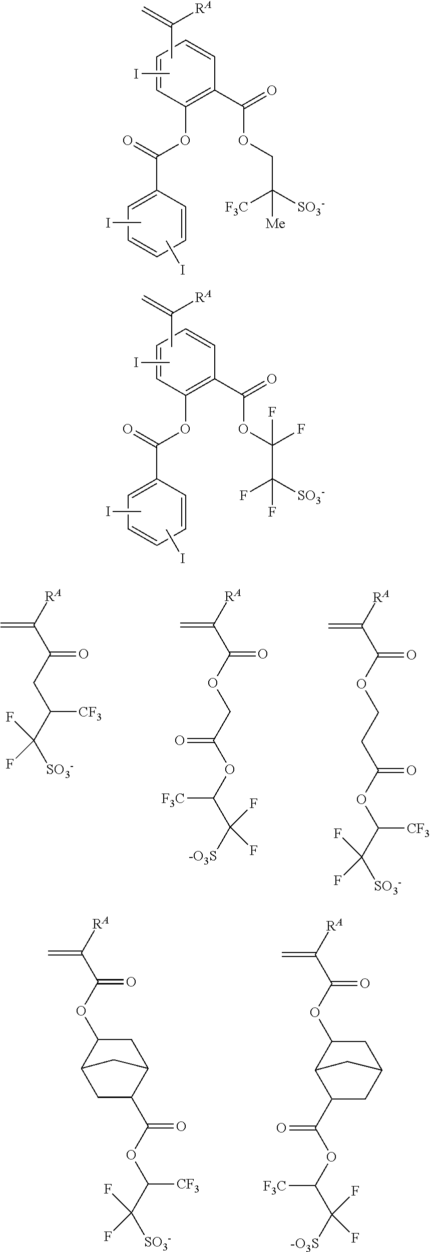

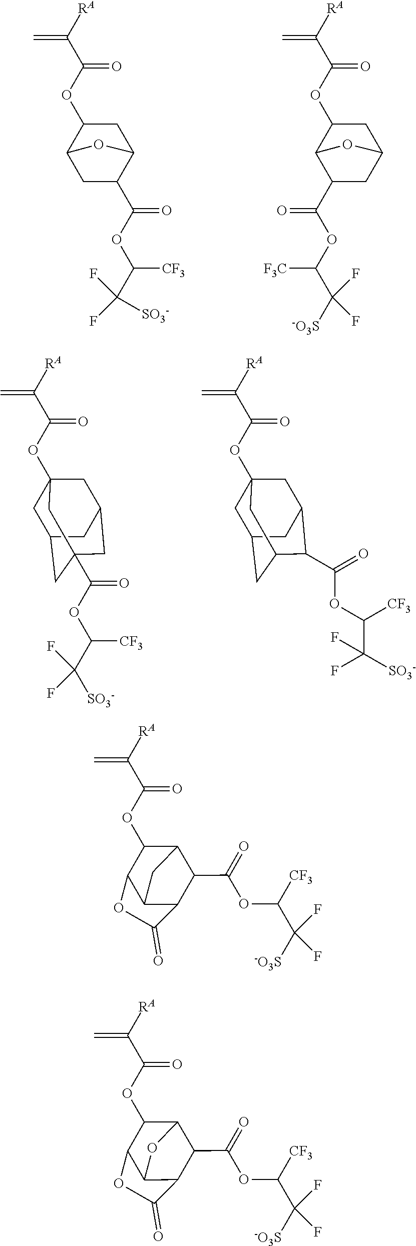

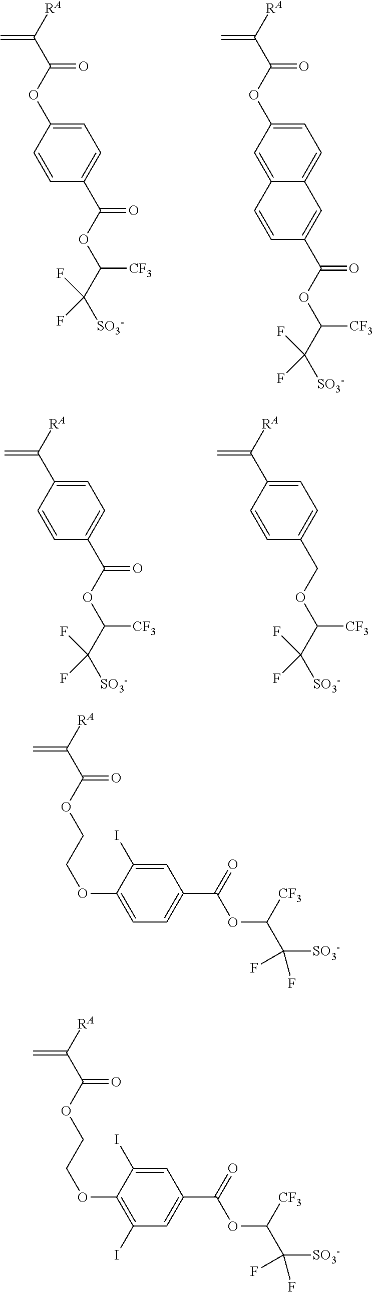

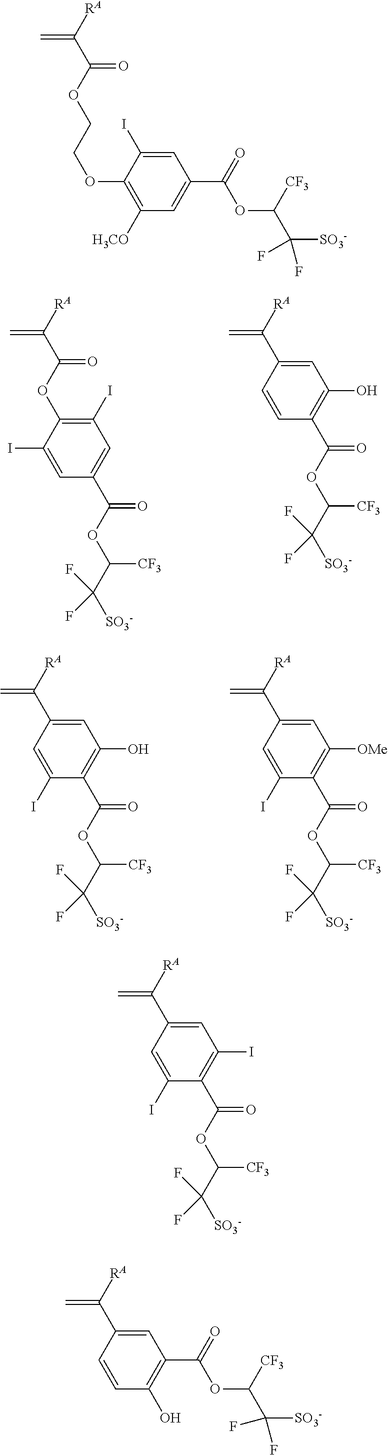

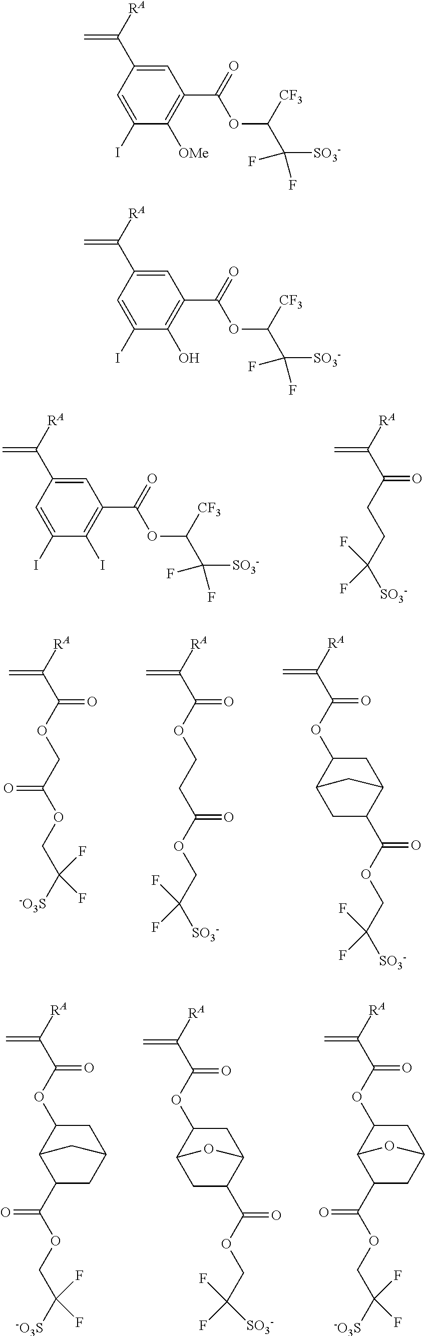

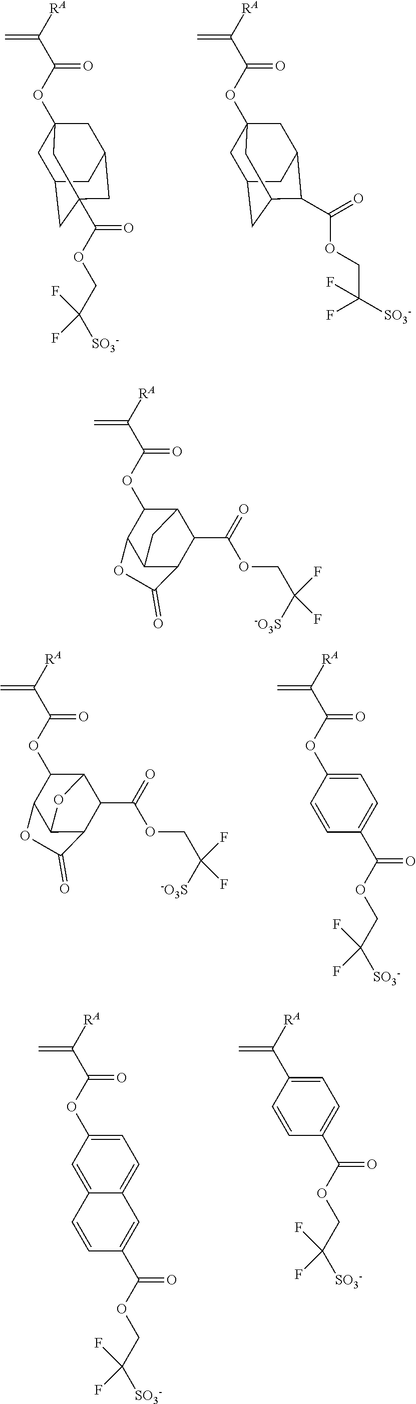

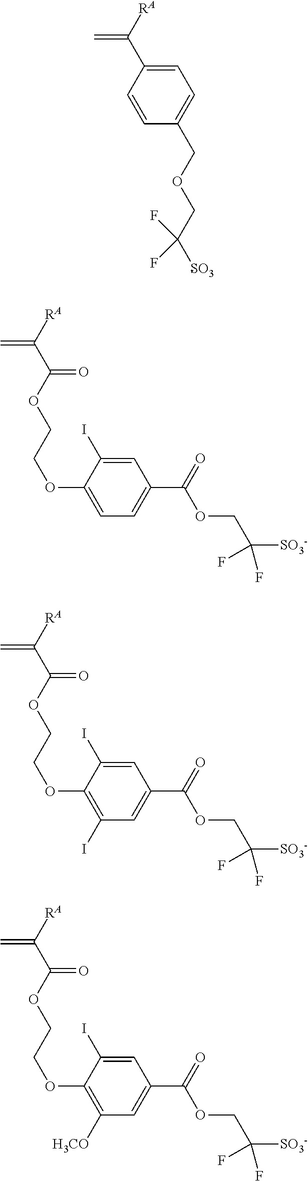

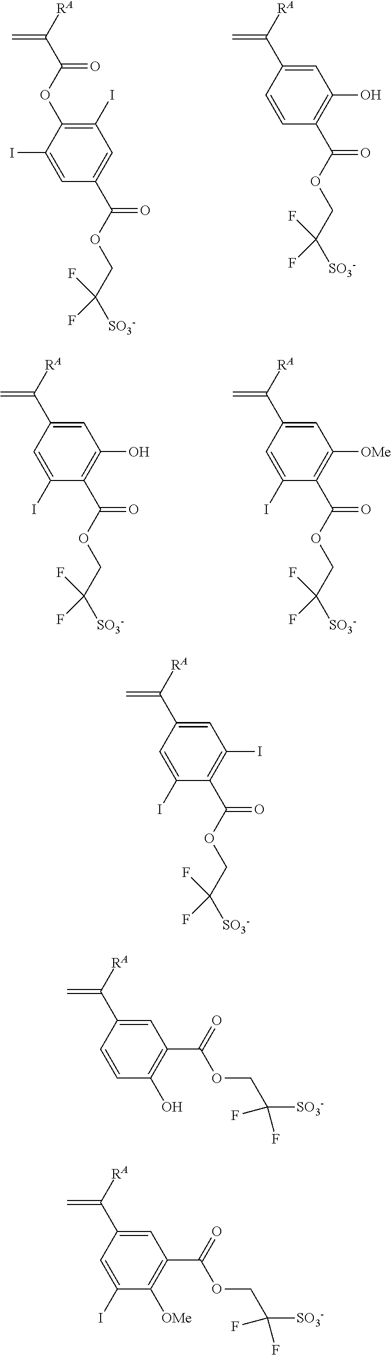

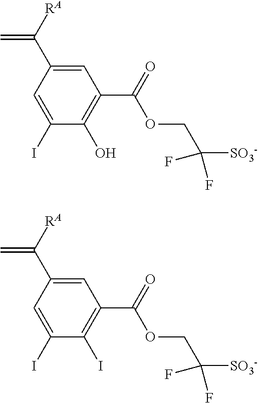

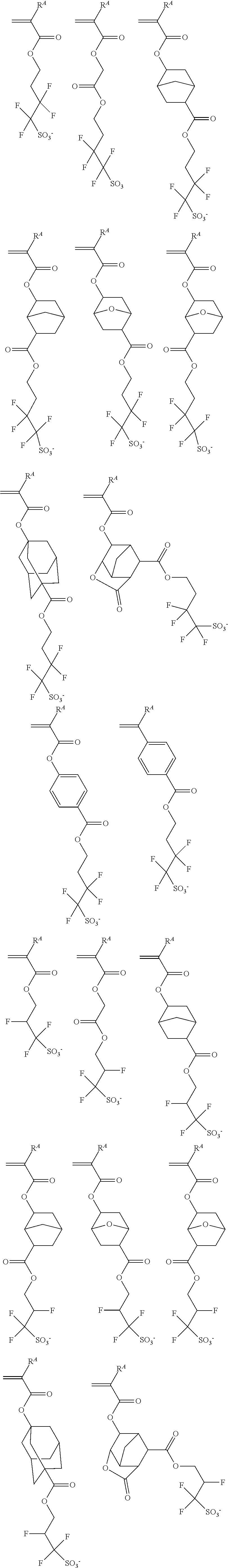

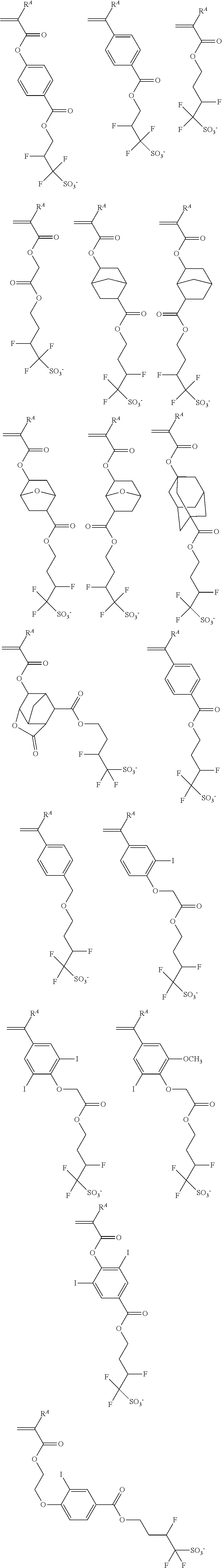

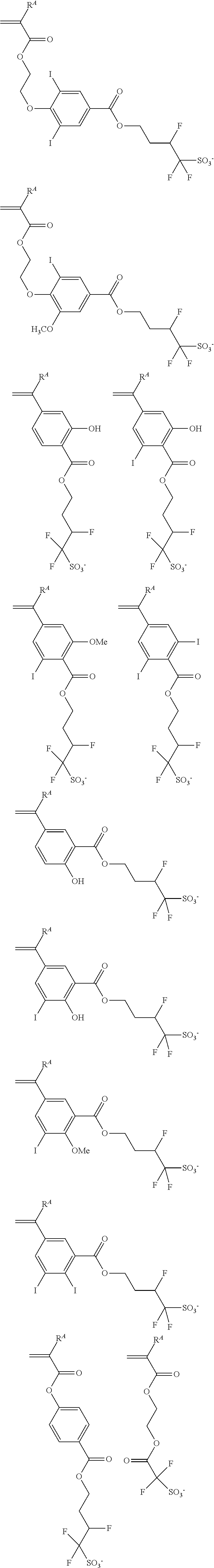

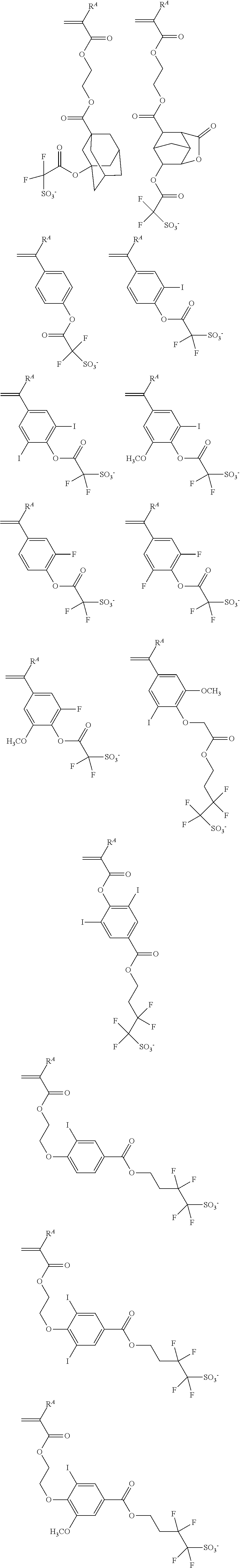

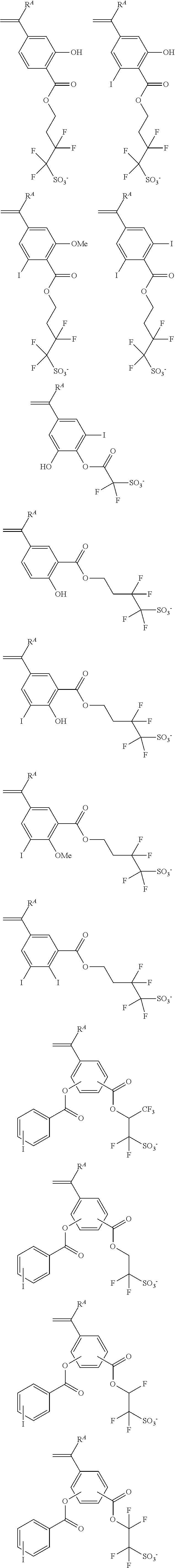

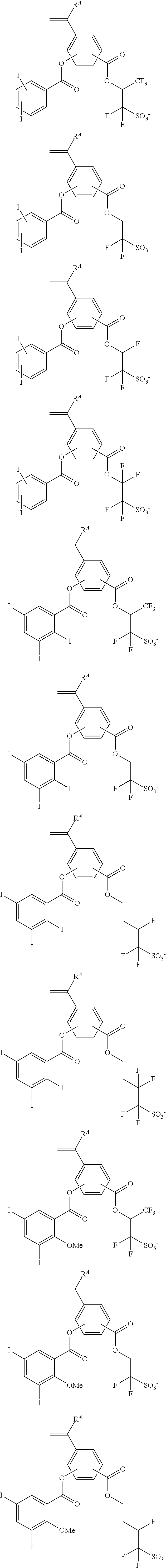

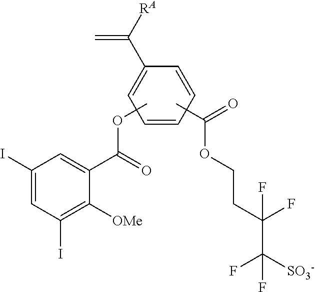

Other examples of the anion Z⁻ are as described in WO 2024/176672, paragraphs [0043]-[0044], WO 2024/176701, paragraphs [0067]-[0070], WO 2024/190386, paragraphs [0057]-[0058], JP-A 2024-103465, paragraphs [0020]-[0023], JP-A 2024-120703, paragraphs [0067]-[0070], JP 7520258, paragraphs [0075]-[0084], JP-A 2024-043941, paragraphs [0032]-[0047], JP-A 2025-013053, paragraphs [0385]-[0390], and WO 2025/018169, paragraphs [0062]-[0064].

Examples of the sulfonium salt monomer of the invention include combinations of anions with cations, both as exemplified above.

The sulfonium salt monomer can be synthesized by any well-known methods. For example, a salt having the above-mentioned sulfonium cation, which is synthesized by the standard method, can be converted to a desired sulfonium salt by subjecting it to salt exchange reaction with a salt having the above-mentioned anion. The salt exchange reaction with the corresponding anion is readily carried out by the standard technique, for example, with reference to JP-A 2007-145797. The above-mentioned preparation method is merely exemplary, and the method of preparing the sulfonium salt of the invention is not limited thereto.

A structural characteristic of the sulfonium salt monomer of the invention is that the sulfonium salt monomer has a fluoroalkanesulfonate anion having a polymerizable group, and a triarylsulfonium cation having, on the same aromatic ring, a cyano group and an acid labile group having a tertiary/secondary ether structure, a tertiary/secondary ester structure, a tertiary/secondary carbonate structure or an acetal structure. In the sulfonium salt monomer of the invention, the anion has a polymerizable group. Therefore, the polymer of the invention obtained using the sulfonium salt monomer provides an anion-bound acid generator in which the anion is bonded to the polymer backbone. That is, since an acid bonded to the polymer backbone is generated, diffusion of the generated acid can be suppressed. In particular, a polymerizable group having a styrene or vinylnaphthalene structure is preferred because it has higher rigidity over a polymerizable group such as a methacrylic acid ester, and improves the glass transition temperature (Tg) of the polymer. It is considered that the aromatic rings in the polymer or between the polymers interact with each other (exhibiting a π-π stacking effect) to regularly arrange the polymers, and even in fine pattern formation, resistance to pattern collapse is exhibited against the developer. In an etching step after fine pattern formation, excellent etch resistance is also exhibited because the aromatic ring is directly bound to the backbone.

The sulfonium salt monomer of the invention preferably has an iodine atom in the anion. With regard to an iodine atom, an iodine atom which is highly absorptive to EUV generates secondary electrons upon exposure particularly in the EUV lithography of wavelength 13.5 nm. The cyano bound to the aromatic ring of the sulfonium cation is known as a strong electron-withdrawing group. From this, it is considered that the energy level of the LUMO of the frontier orbital theory decreases. Therefore, secondary electrons generated from the iodine atom in the anion are easily received, so that the decomposition of the cation is promoted and the acid is effectively generated. In addition, since the nitrogen atom of cyano has a lone pair of electrons, it is also expected to interact with a proton of a generated acid and thereby function as an acid diffusion-controlling group. The acid labile group having a tertiary/secondary ether structure, a tertiary/secondary ester structure, a tertiary/secondary carbonate structure, or an acetal structure and bonded on the same aromatic ring in the sulfonium cation undergoes a deprotection reaction with an acid generated by exposure, and generates a corresponding phenol or carboxylic acid. The structure changes from a lipophilic structure to a hydrophilic structure to improve the contrast between an exposed portion and an unexposed portion. Cyano is bonded to a carbon atom adjacent to a carbon atom to which a phenolic hydroxy group or a carboxy group formed by a deprotection reaction is attached, so that the acidity of a phenol or a carboxylic acid is improved due to the electron-withdrawing property of the cyano. When the exposed portion in which the deprotection reaction has proceeded is developed with an alkaline developer, the phenol having improved acidity has improved solubility in the alkaline developer, so that development residues can be suppressed. Other aromatic rings in the triarylsulfonium cation preferably have thereon a fluorine atom or a substituent containing a fluorine atom. The fluorine atom or the substituent containing a fluorine atom, which has a strong electron-withdrawing property, thus may lower the energy level of the LUMO of the frontier orbital theory. Therefore, secondary electrons generated from the iodine atom in the cation and the anion are easily received, so that the decomposition of the cation is promoted and the acid is effectively generated. The synergy of these effects results in a higher sensitivity, and any lowering of resolution due to blur by acid diffusion can be prevented, contributing an improvement in lithography performance such as LWR and CDU. Accordingly, the polymer of the invention is particularly suitable as a material for chemically amplified positive resist compositions.

### [Polymer]

In another aspect, the invention provides a polymer comprising repeat units derived from the sulfonium salt monomer having formula (A) (hereinafter, also referred to as repeat units A).

The polymer may comprise repeat units having the formula (a1) (hereinafter, also referred to as repeat units a1) or repeat units having the formula (a2) (hereinafter, also referred to as repeat units a2).

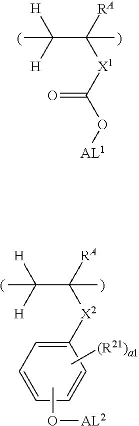

In formulae (a1) to (a2), R^{A} is each independently hydrogen, fluorine, methyl or trifluoromethyl.

In formula (a1), X¹ is a single bond, phenylene group, naphthylene group, *—C(═O)—O—X¹¹— or *—C(═O)—N(H)—X¹¹—, and the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen. X¹¹ is a C₁-C₁₀ saturated hydrocarbylene group, phenylene group or naphthylene group, and the saturated hydrocarbylene group may contain hydroxy, ether bond, ester bond or lactone ring. * designates a point of attachment to the carbon atom in the backbone.

In formula (a2), X² is a single bond, *—C(═O)—O— or *—C(═O)—N(H)—. * designates a point of attachment to the carbon atom in the backbone. R²¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom. a1 is 0, 1, 2, 3 or 4, preferably 0 or 1. When a1 is 2, 3 or 4, a plurality of R²¹ may be identical or different.

In formulae (a1) and (a2), AL¹ and AL² are each independently an acid labile group. Examples of the acid labile group are as described in JP-A 2013-080033 and JP-A 2013-083821.

Typical of the acid labile group are groups having the formulae (AL-3) to (AL-5).

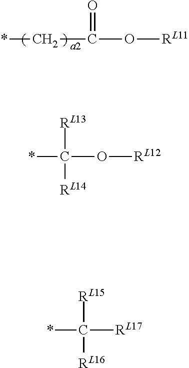

Herein, * designates a point of attachment.

In formulae (AL-3) and (AL-4), R^{L11} and R^{L12} are each independently a C₁-C₄₀ hydrocarbyl group which may contain a heteroatom such as oxygen, sulfur, nitrogen, or fluorine. The hydrocarbyl group may be saturated or unsaturated, and may be straight, branched or cyclic. The hydrocarbyl group is preferably a C₁-C₂₀ hydrocarbyl group.

In formula (AL-3), a2 is an integer of 0 to 10, preferably 1, 2, 3, 4 or 5.

In formula (AL-4), R^{L13} and R^{L14} are each independently a hydrogen atom or a C₁-C₂₀ hydrocarbyl group which may contain a heteroatom such as oxygen, sulfur, nitrogen, or fluorine. The hydrocarbyl group may be saturated or unsaturated, and may be straight, branched or cyclic. Any two of R^{L}12, R^{L13}, and R^{L14} may bond together to form a C₃-C₂₀ ring with the carbon atom or carbon and oxygen atoms to which they are attached. The ring is preferably a C₄-C₁₆ ring, particularly preferably in an alicyclic form.

In formula (AL-5), R^{L1}, R^{L16} and R^{L17} are each independently a C₁-C₂₀ hydrocarbyl group which may contain a heteroatom such as oxygen, sulfur, nitrogen, or fluorine. The hydrocarbyl group may be saturated or unsaturated, and may be straight, branched or cyclic. Any two of R^{L15}, R^{L16} and R^{L17} may bond together to form a C₃-C₂₀ ring with the carbon atom to which they are attached. The ring is preferably a C₄-C₁₆ ring, particularly preferably in an alicyclic form.

Examples of the acid labile group are as described in JP-A 2023-123222, paragraphs [0064]-[0068], and JP 7492842, paragraphs [0013]-[0014]. These are obtained through a reaction driven to proceed by generation of a conjugated olefin or an acrylic acid ester derivative after the acid elimination reaction.

Examples of repeat unit a1 are shown below, but are not limited thereto. Herein, R^{A} and AL¹ are as defined above.

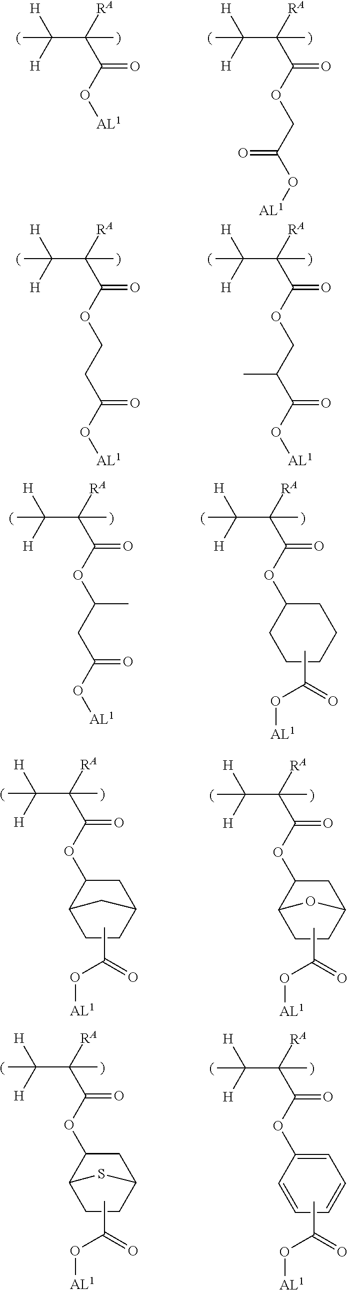

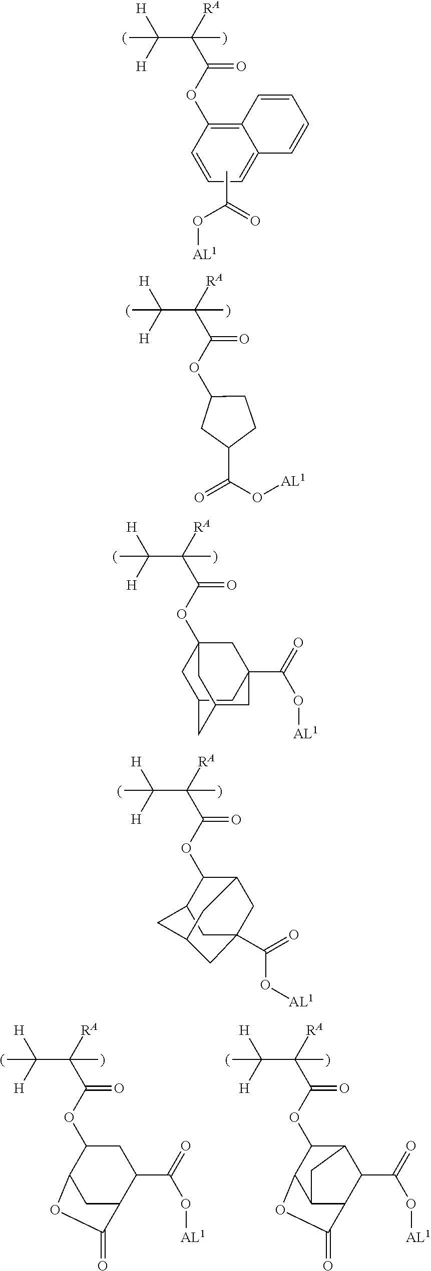

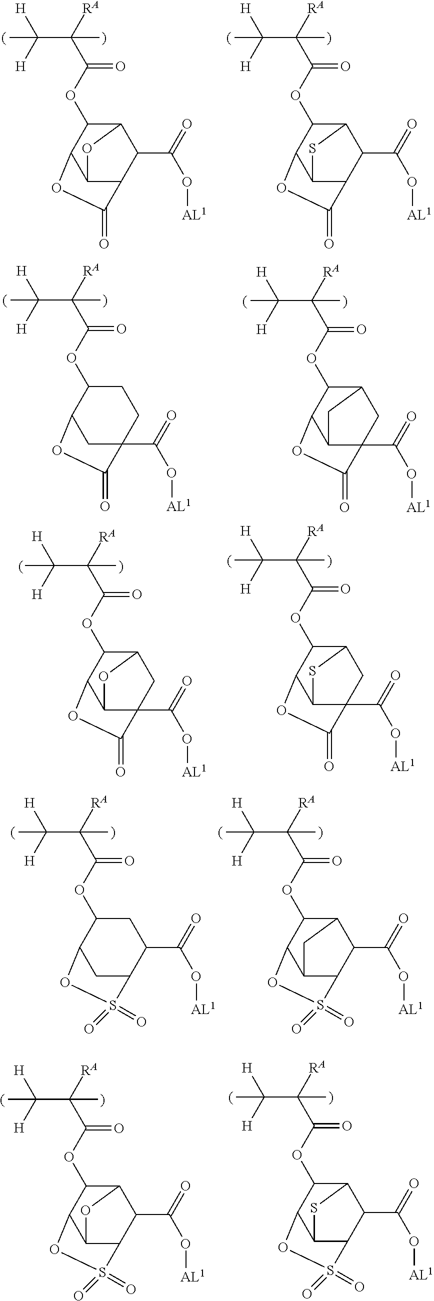

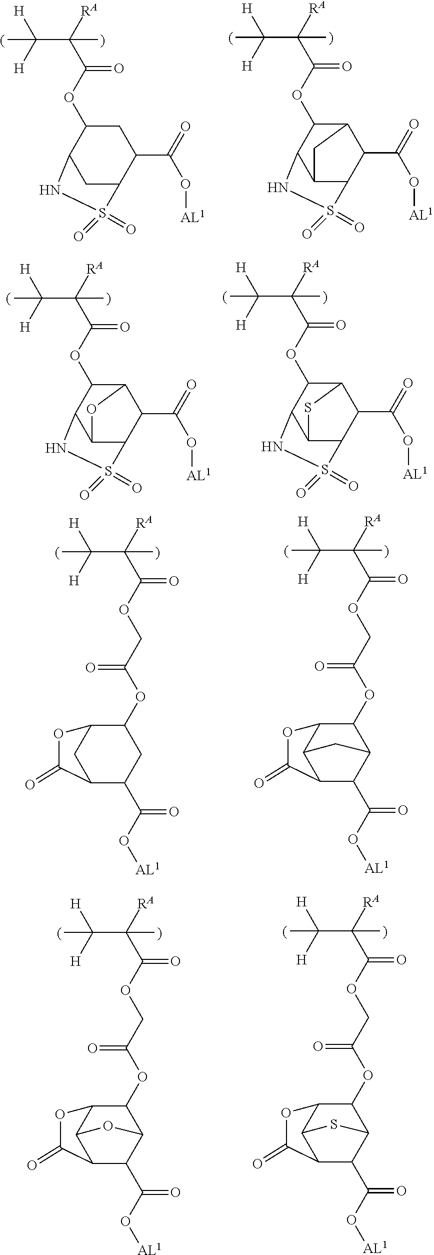

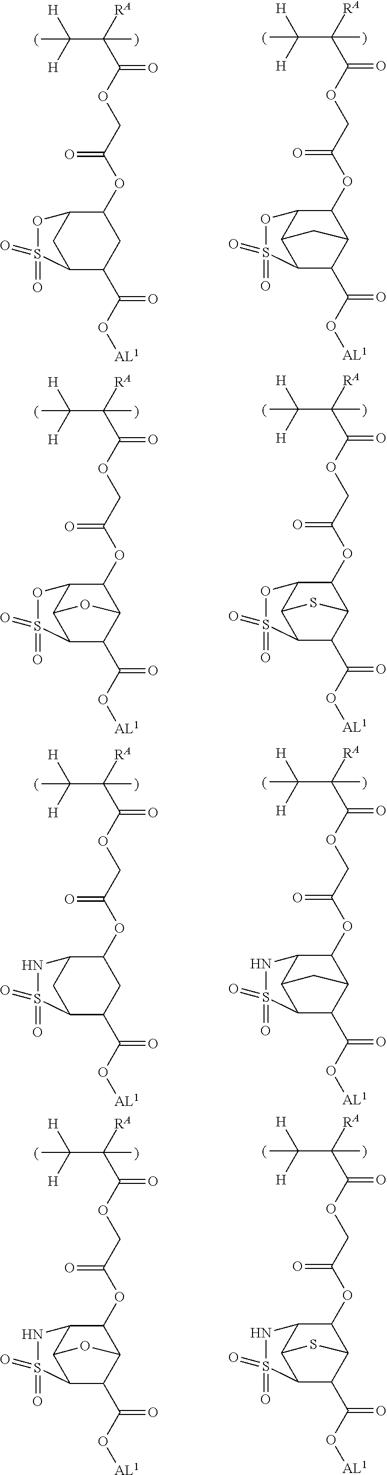

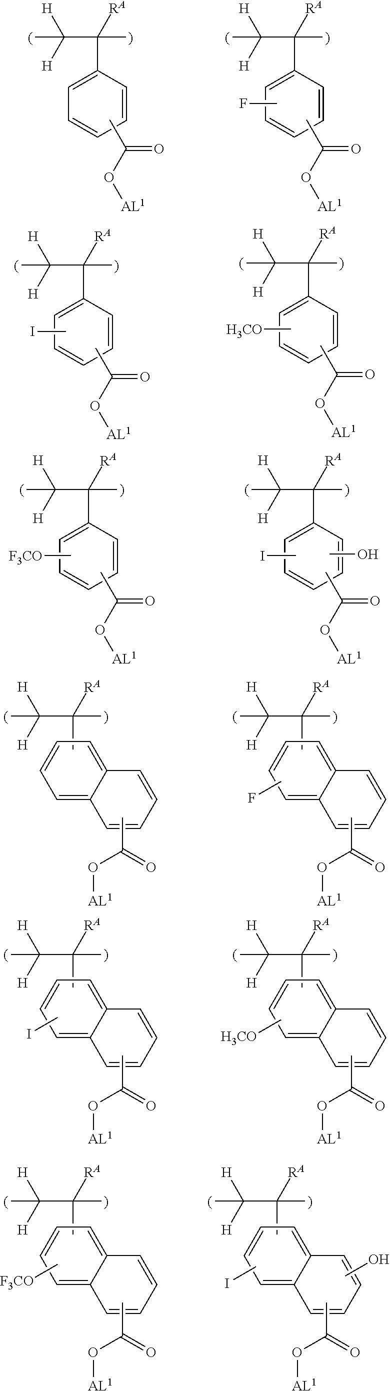

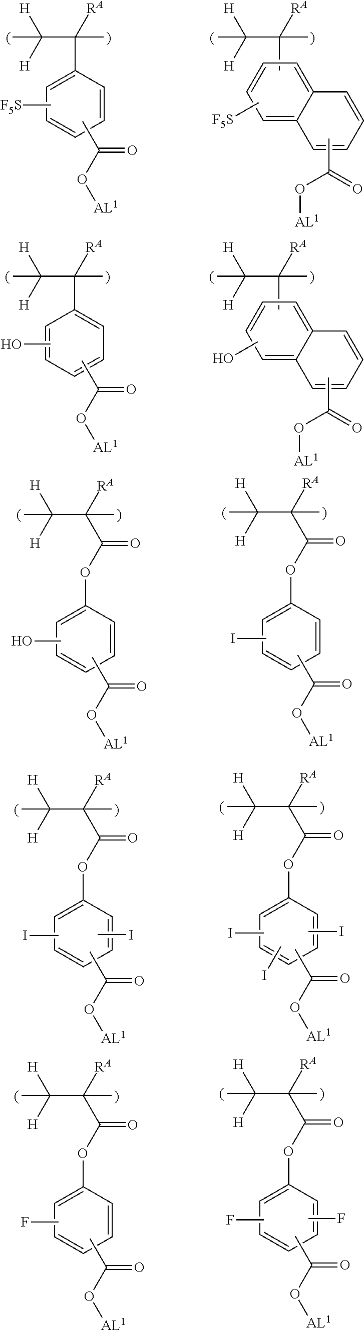

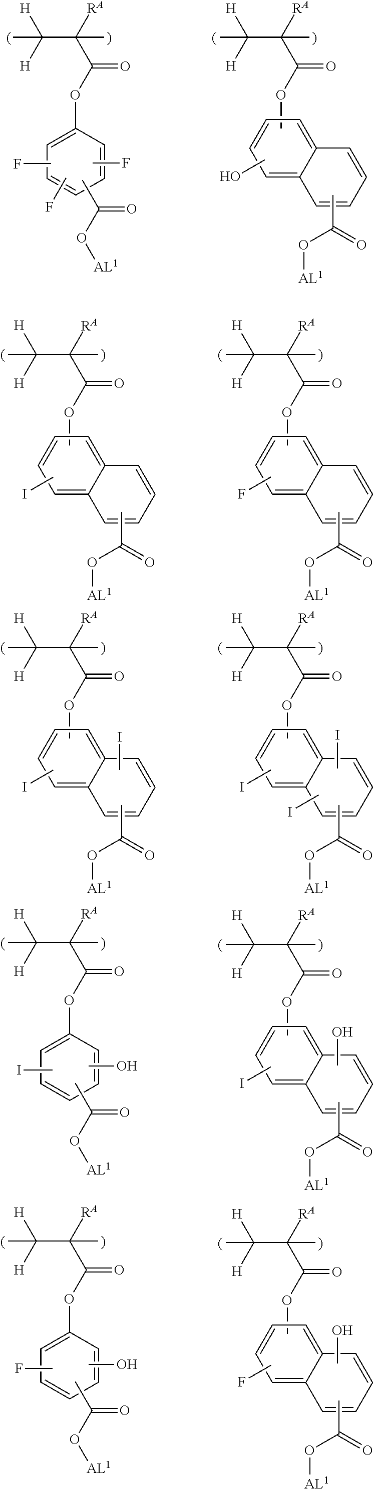

Examples of repeat unit a2 are shown below, but are not limited thereto. Herein, R^{A} and AL² are as defined above.

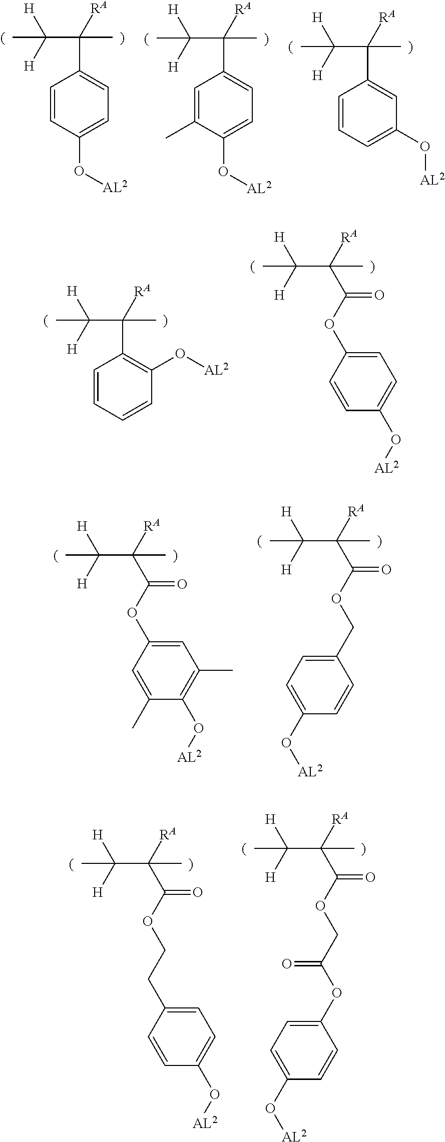

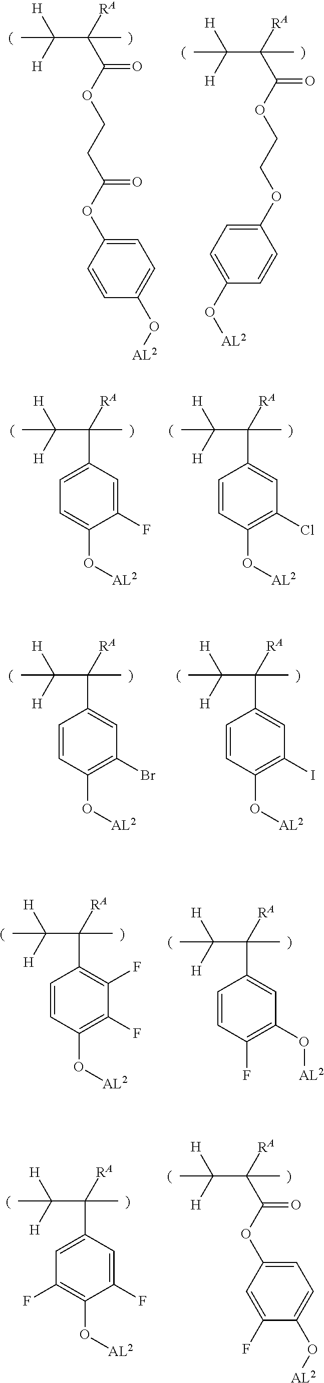

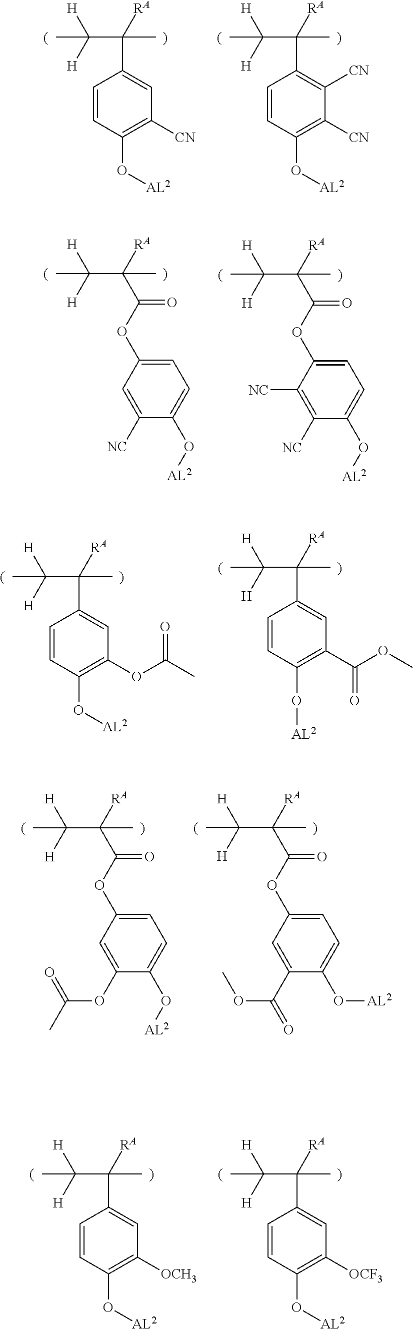

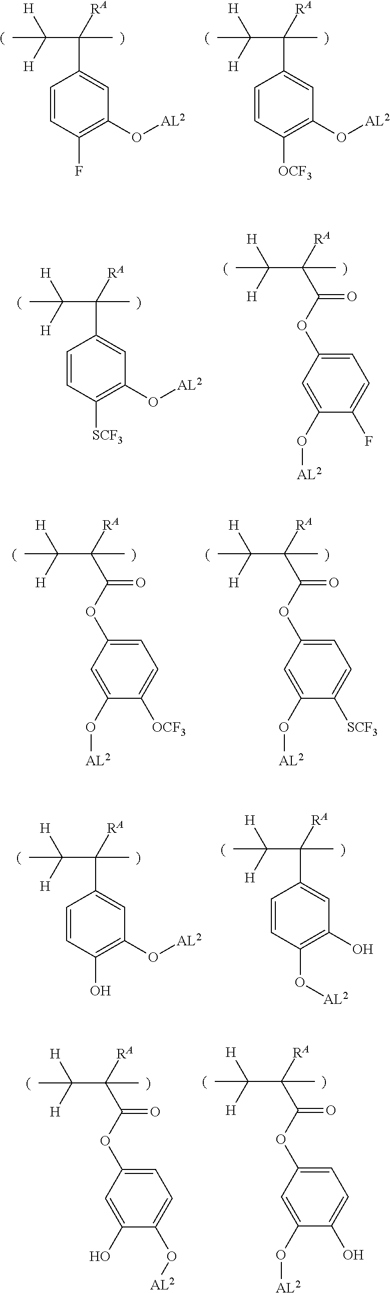

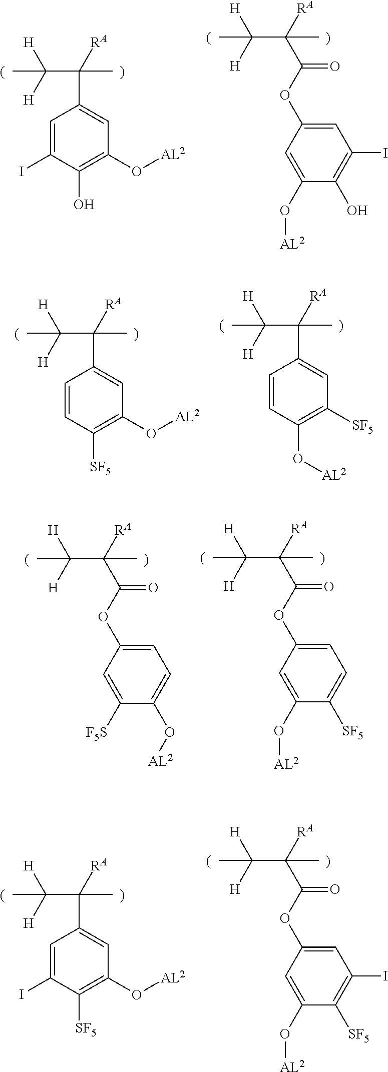

The polymer may comprise repeat units having the formula (a3) (hereinafter, also referred to as repeat units a3).

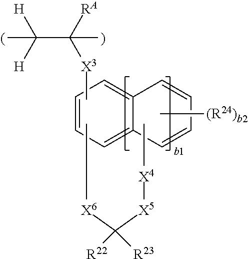

In formula (a3), b1 is 0 or 1. The relevant structure is a benzene ring when b1=0 and a naphthalene ring when b1=1. From the aspect of solvent solubility, the benzene ring corresponding to b1=0 is preferred. b2 is 0, 1, 2 or 3 when b1=0 and 0, 1, 2, 3, 4 or 5 when b1l. It is preferred from the aspect of reactant availability that b2 be 0, 1, 2 or 3, more preferably 0, 1 or 2.

In formula (a3), R^{A} is hydrogen, fluorine, methyl or trifluoromethyl. R^{A} is preferably hydrogen or methyl, more preferably hydrogen.

In formula (a3), X³ is a single bond, *—C(═O)—O— or *—C(═O)—N(H)—. * designates a point of attachment to the carbon atom in the backbone. X³ is preferably a single bond or *—C(═O)—O—, more preferably a single bond.

In formula (a3), X⁴ is a single bond, C₁-C₄ aliphatic hydrocarbylene group, carbonyl group, sulfonyl group or a group obtained by combining the foregoing. It is preferred from the aspect of reactant availability that X⁴ be a single bond, carbonyl, or sulfonyl. It is more preferred from the aspect of a polar group created after reaction that X⁴ be a single bond or carbonyl.

In formula (a3), X⁵ and X⁶ are each independently oxygen or sulfur. Provided that X⁴ and X⁶ are attached to adjacent carbon atoms on the aromatic ring. X⁵ and X⁶ may be identical or different. It is preferred from the aspect of reactivity that X⁵ and X⁶ be both oxygen.

In formula (a3), R²² and R²³ are each independently hydrogen or a C₁-C₂₀ hydrocarbyl group which may contain a heteroatom. The hydrocarbyl group may be saturated or unsaturated, and may be straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, 1-propenyl, 2-propenyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety.

Also, R²² and R²³ may bond together to form a ring with the carbon atom to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur, or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

In formula (a3), R²⁴ is halogen, hydroxy, cyano, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{24A})(R^{24B}). R^{24A} and R^{24B} are each independently hydrogen or a C₁-C₆ hydrocarbyl group. Suitable halogen atoms include fluorine, chlorine, bromine and iodine, with fluorine and iodine being more preferred. The hydrocarbyl group and hydrocarbyl moiety in the hydrocarbyloxy, hydrocarbyloxycarbonyl, and hydrocarbylthio groups may be saturated or unsaturated, and may be straight, branched or cyclic. Examples thereof are as exemplified above for the hydrocarbyl groups R²² and R²³. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety. When b2 is 2 or more, a plurality of R²⁴ may be identical or different.

When b2 is 2 or more, a plurality of R²⁴ may bond together to form a ring with carbon atoms on the aromatic ring to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur, or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

Examples of repeat unit a3 are shown below, but are not limited thereto. Herein, R^{A} is as defined above, and Me is methyl. The positions of attachment of substituents on the aromatic ring are interchangeable.

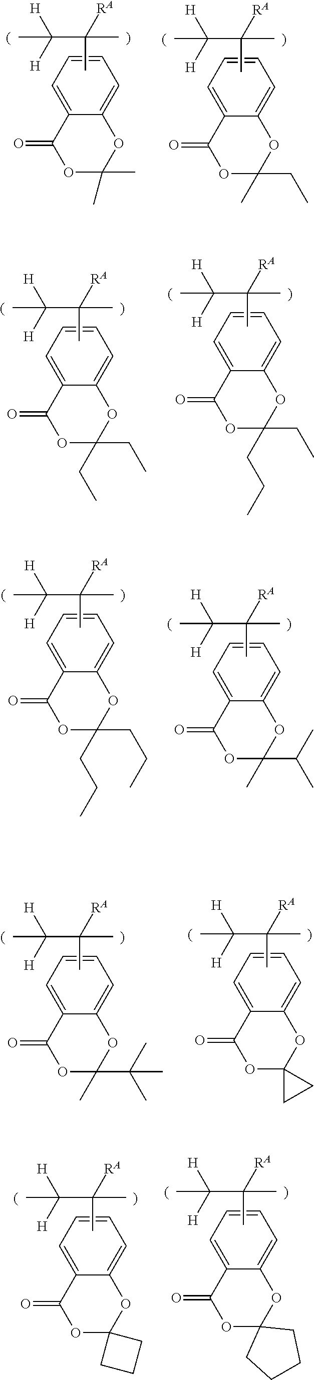

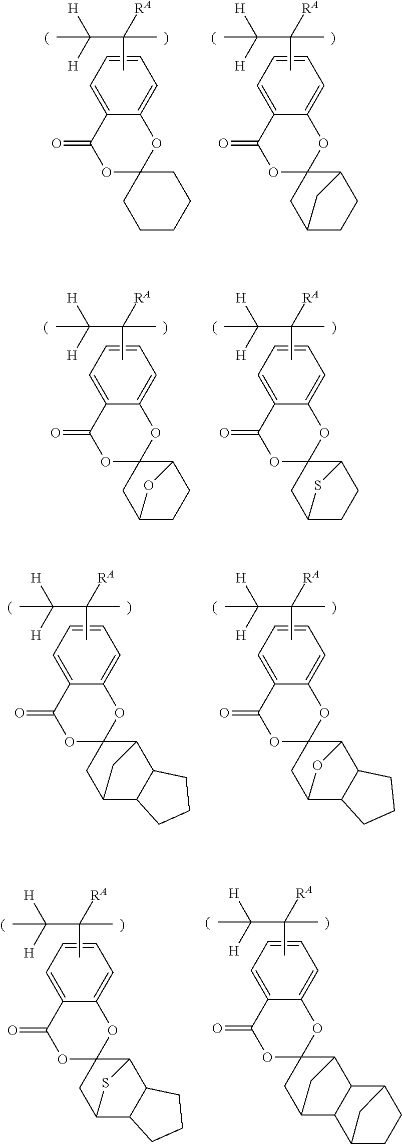

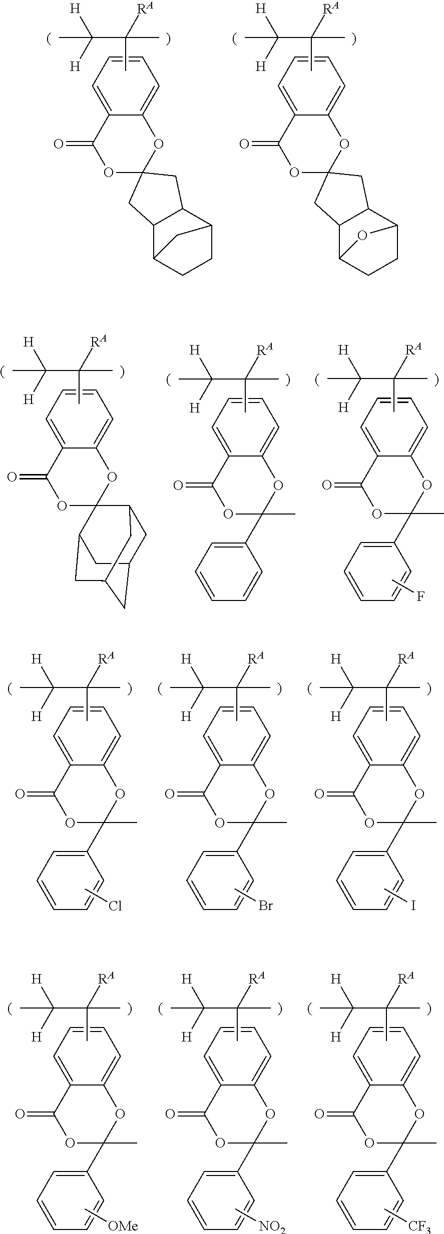

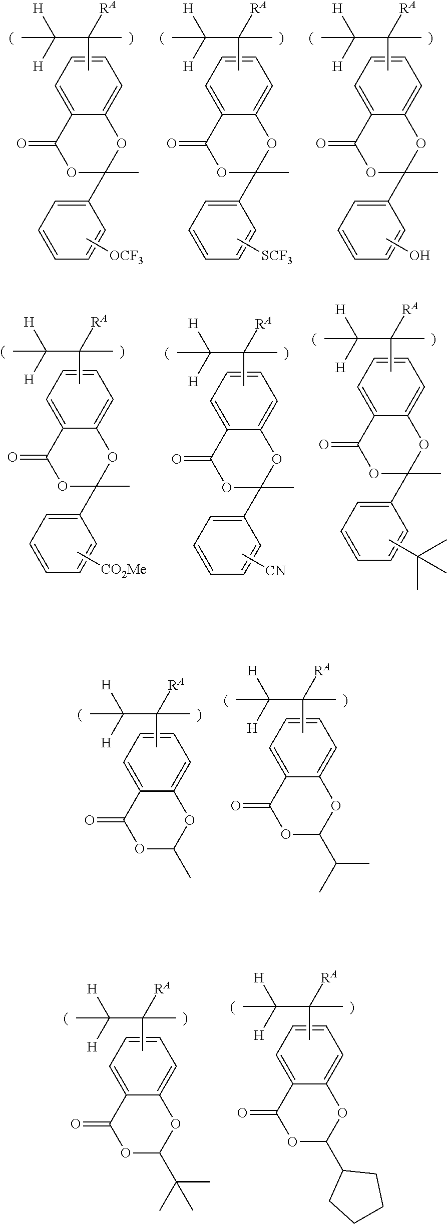

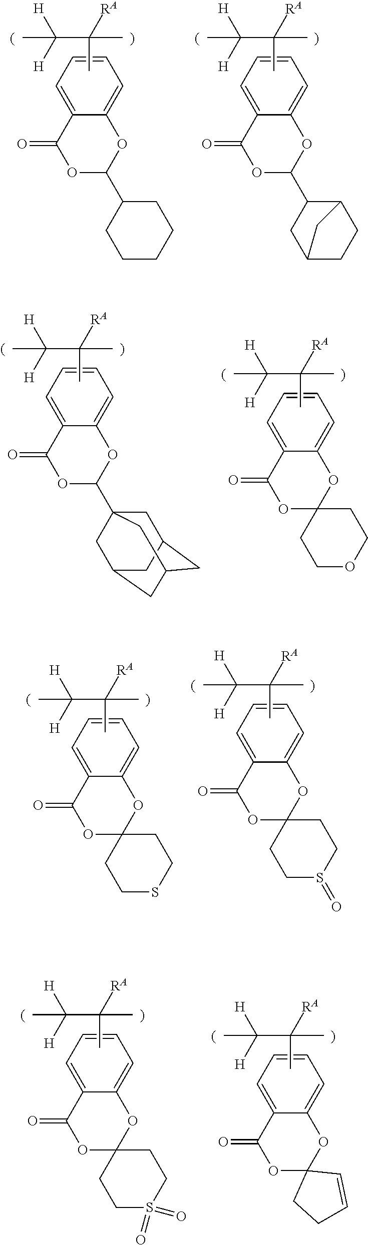

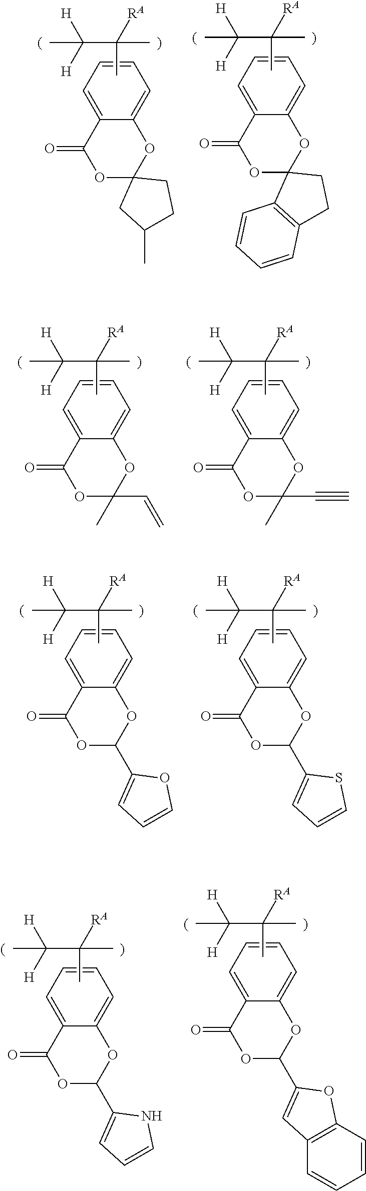

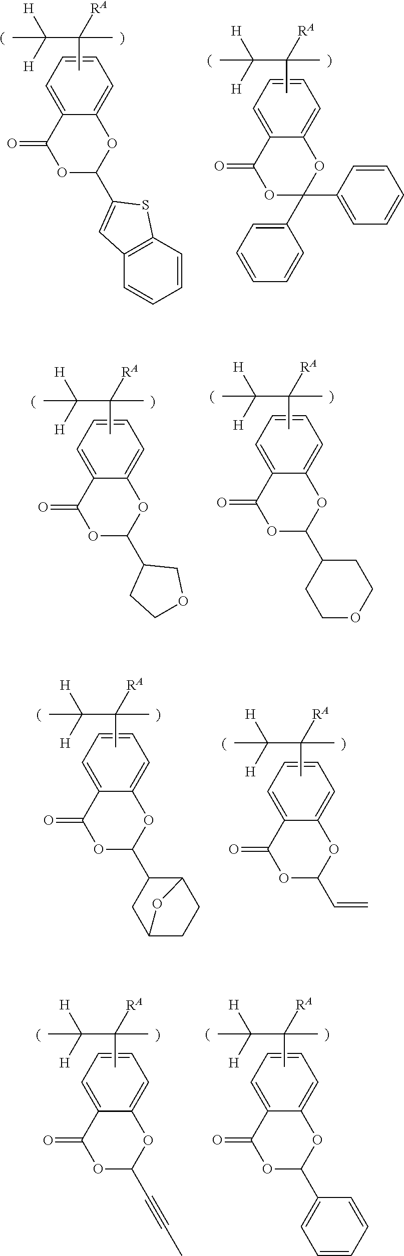

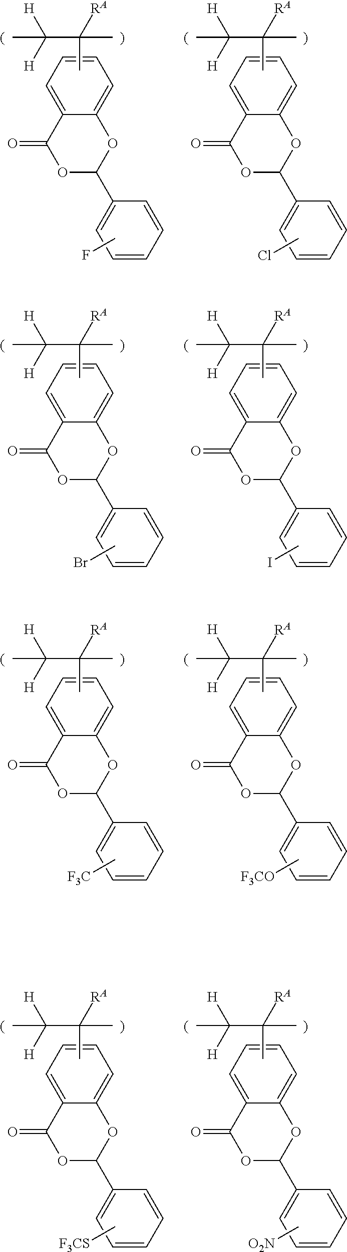

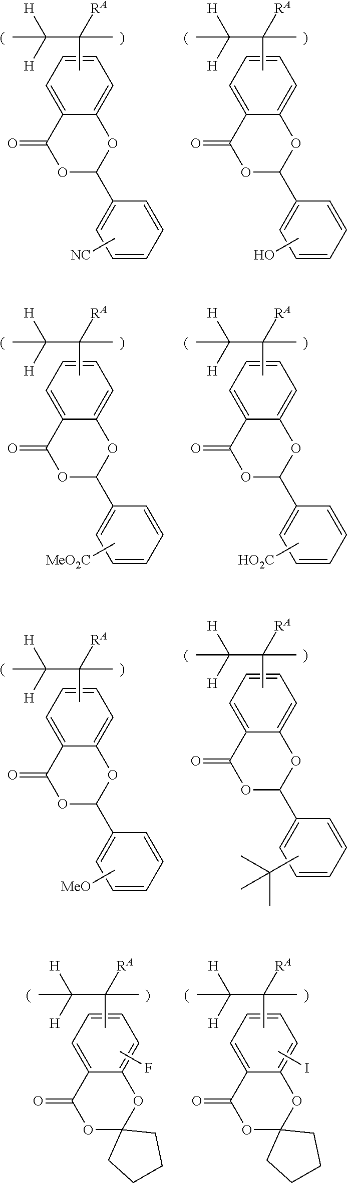

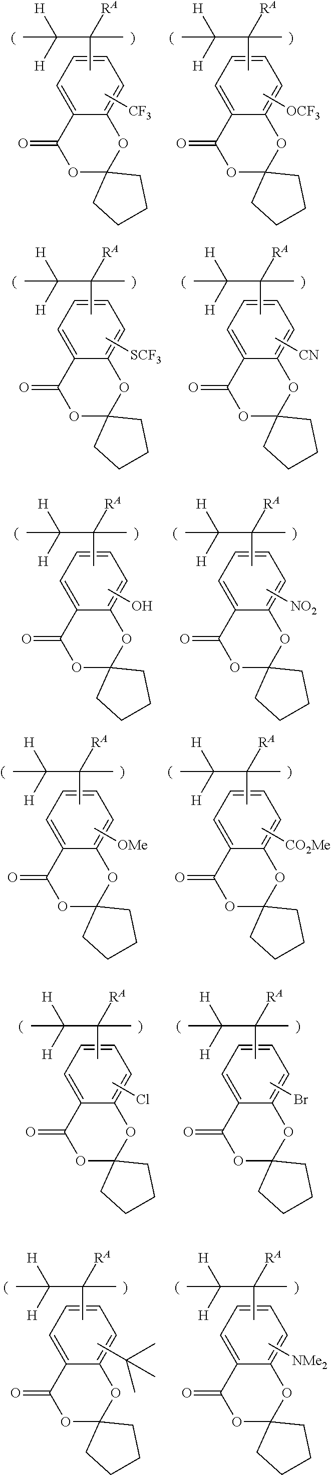

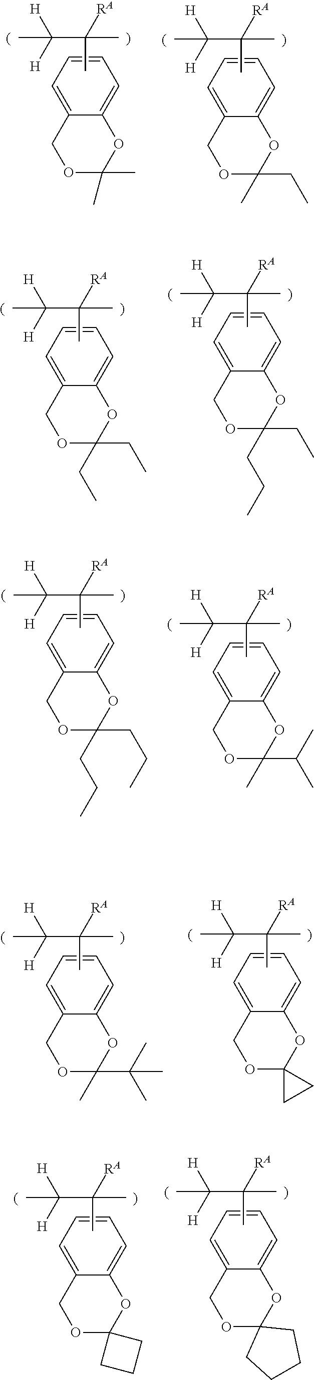

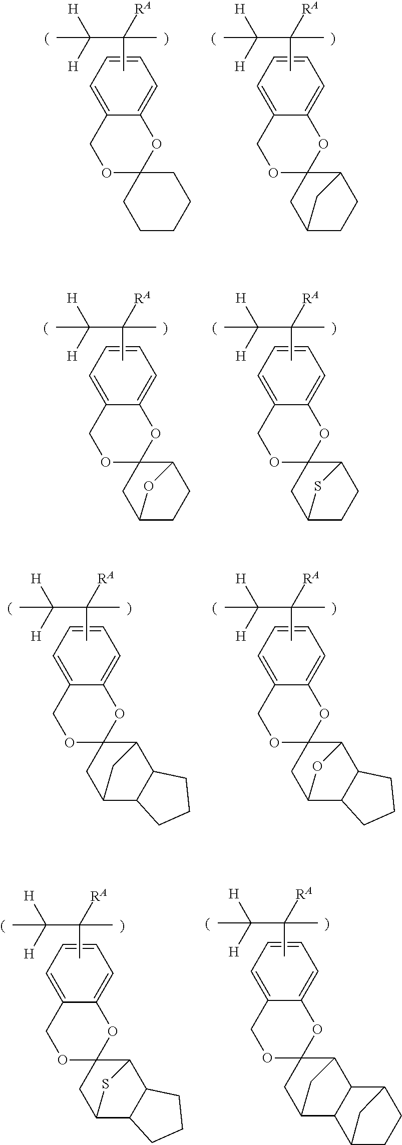

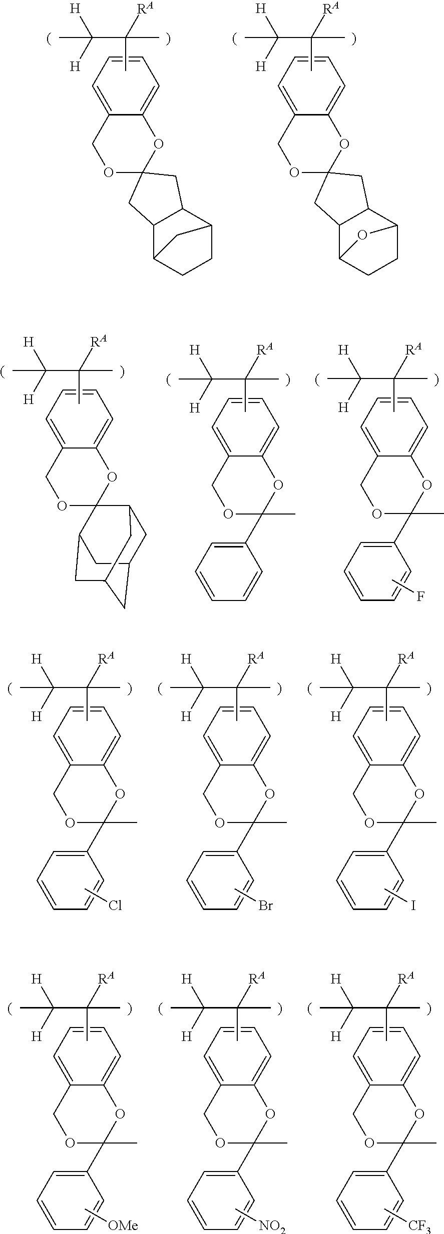

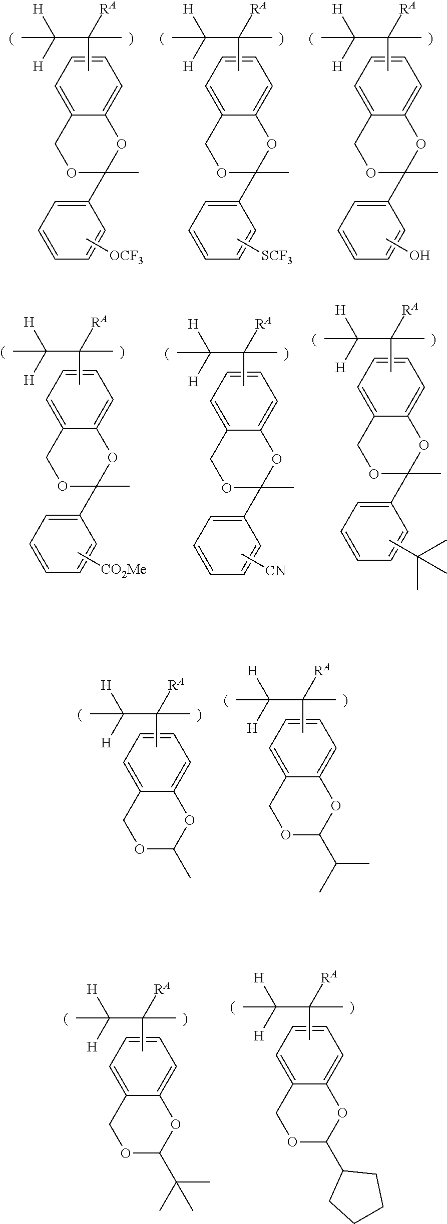

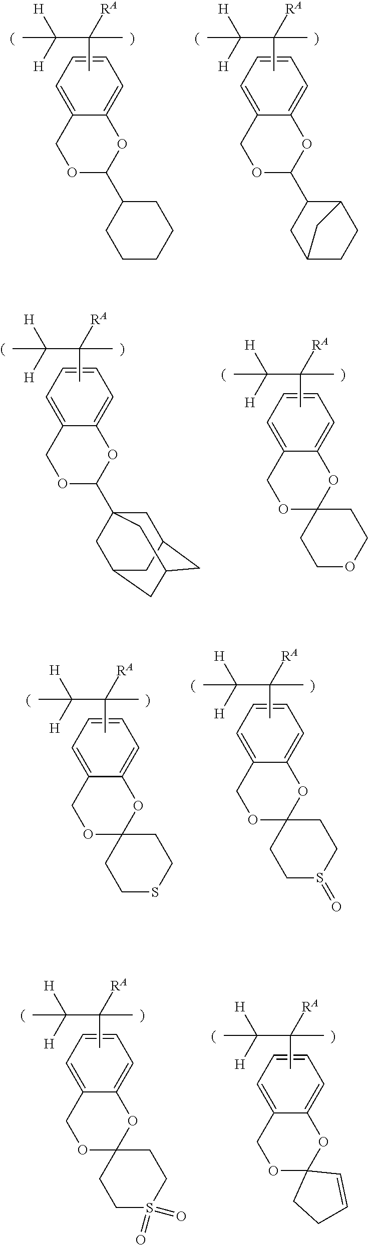

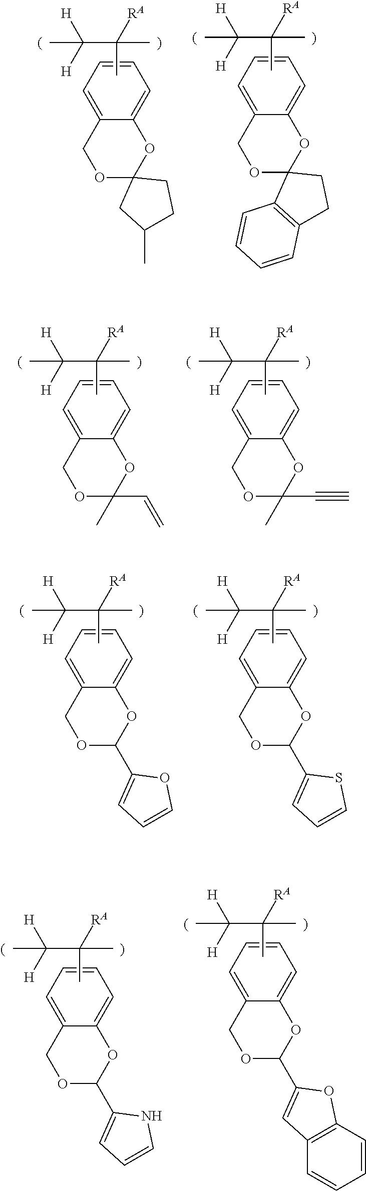

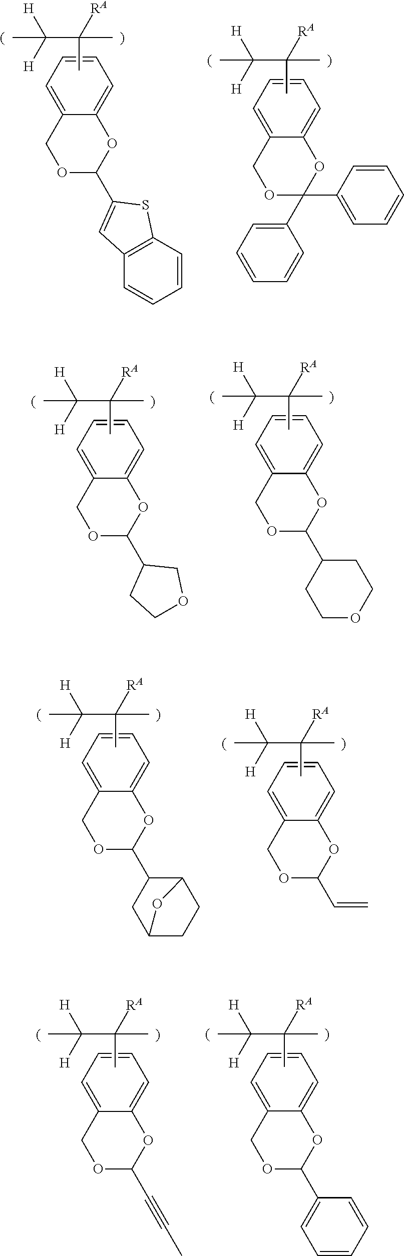

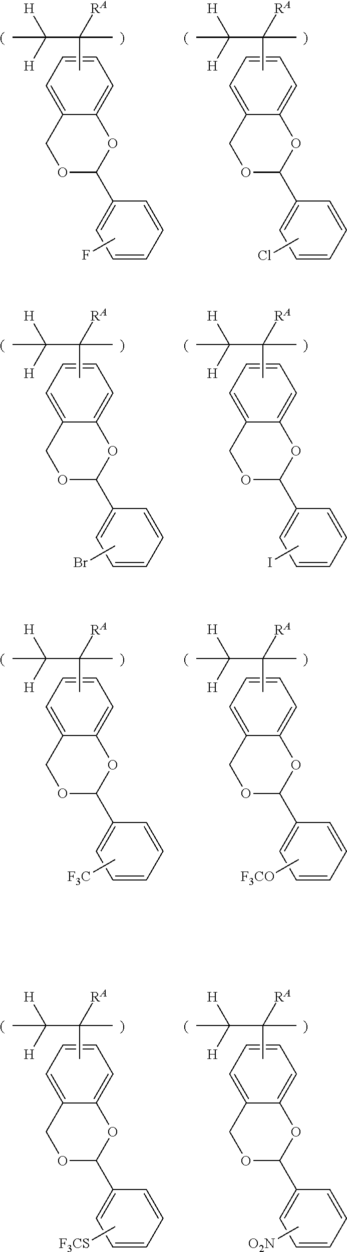

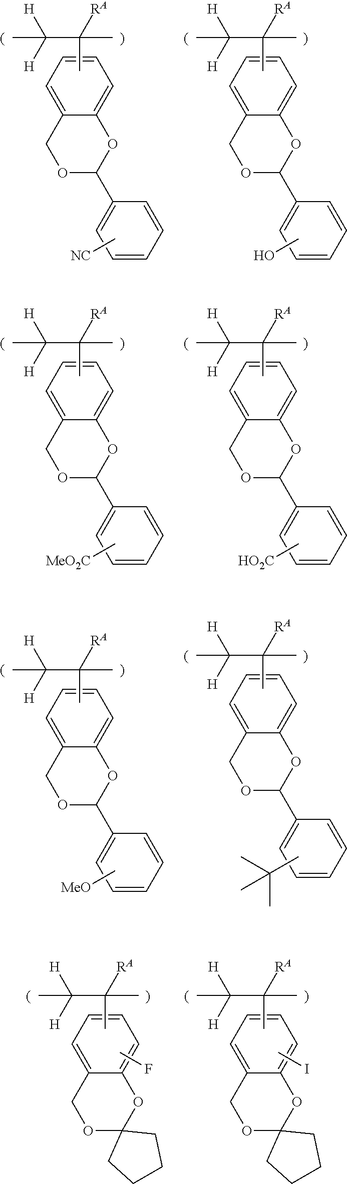

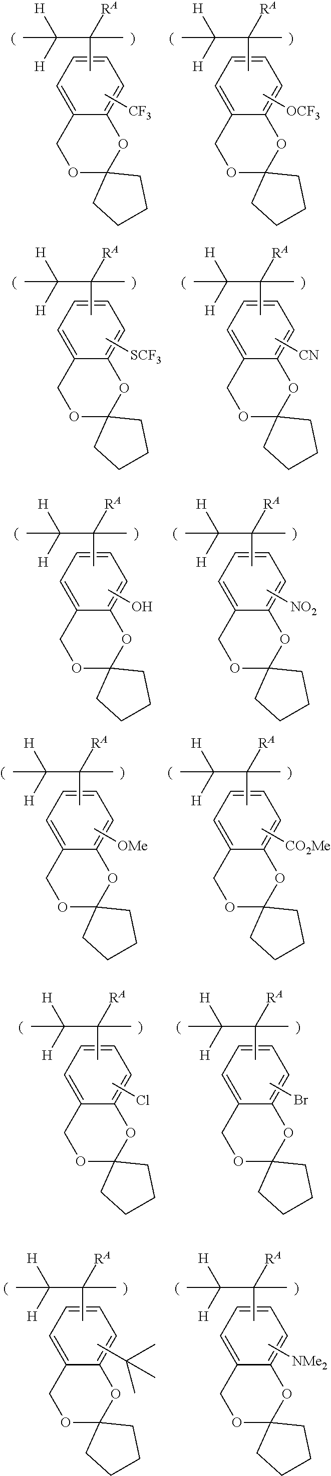

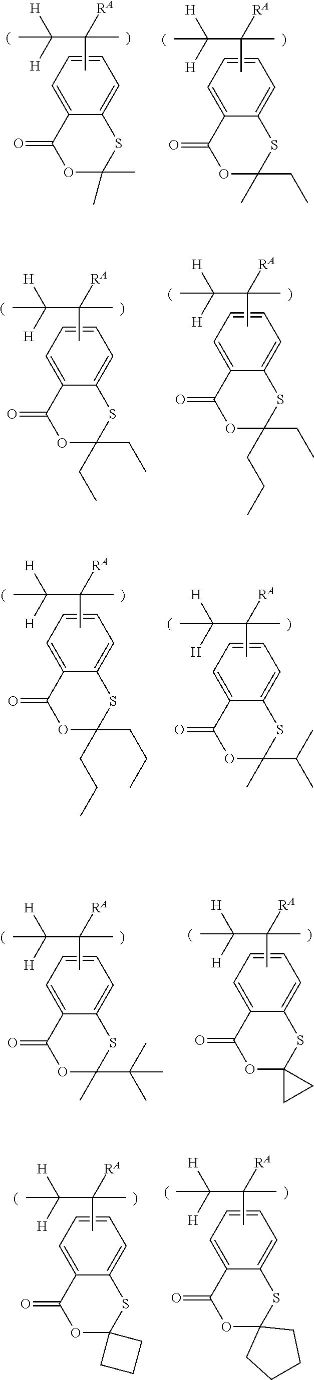

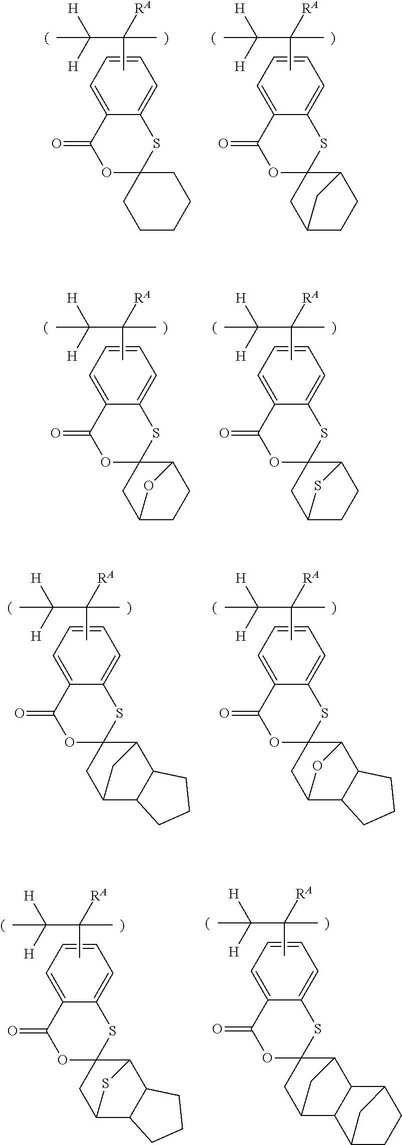

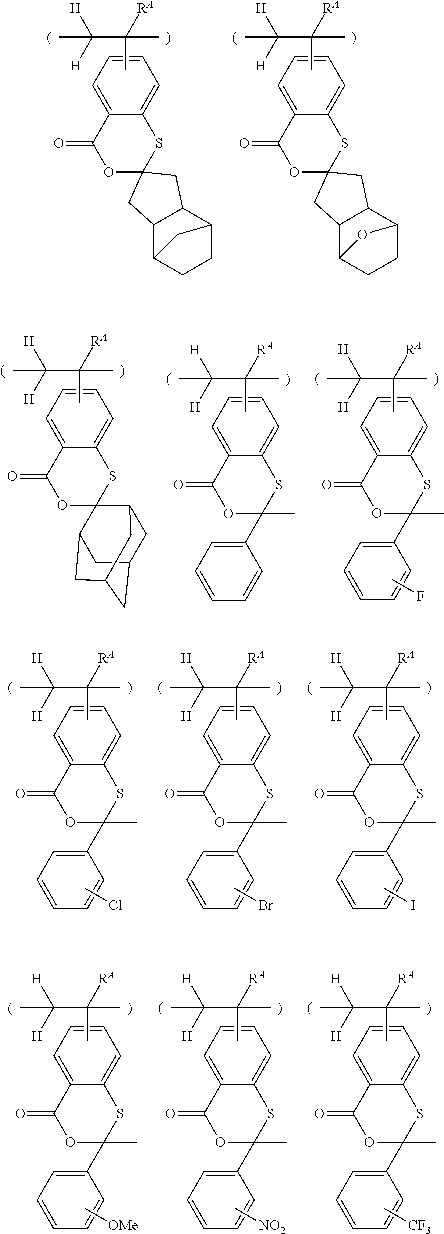

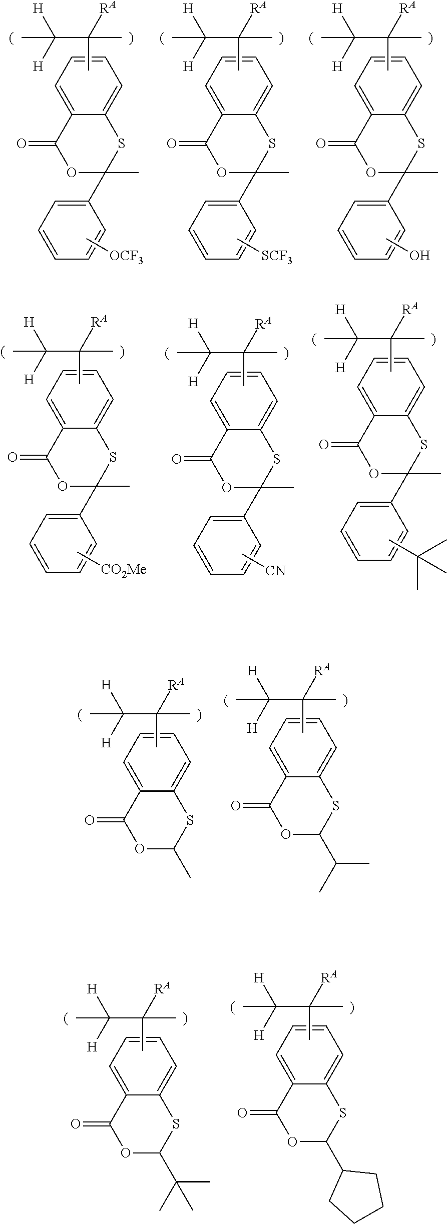

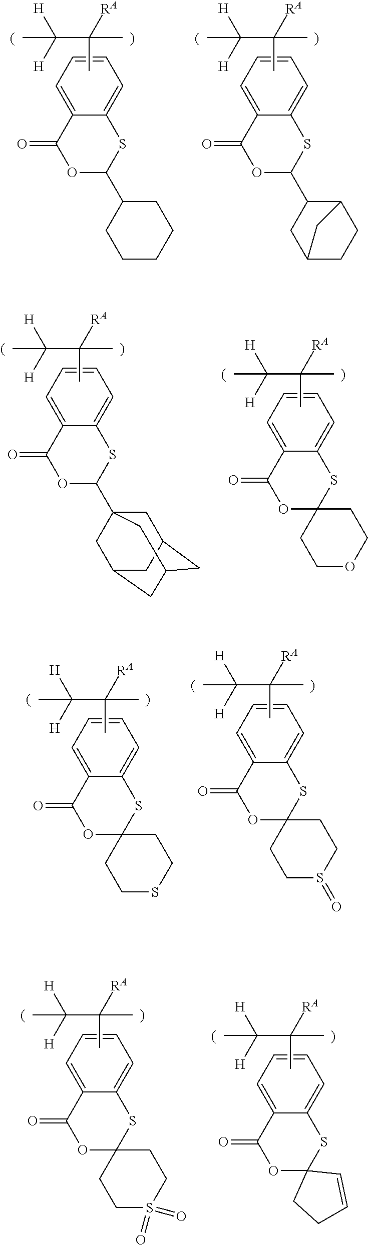

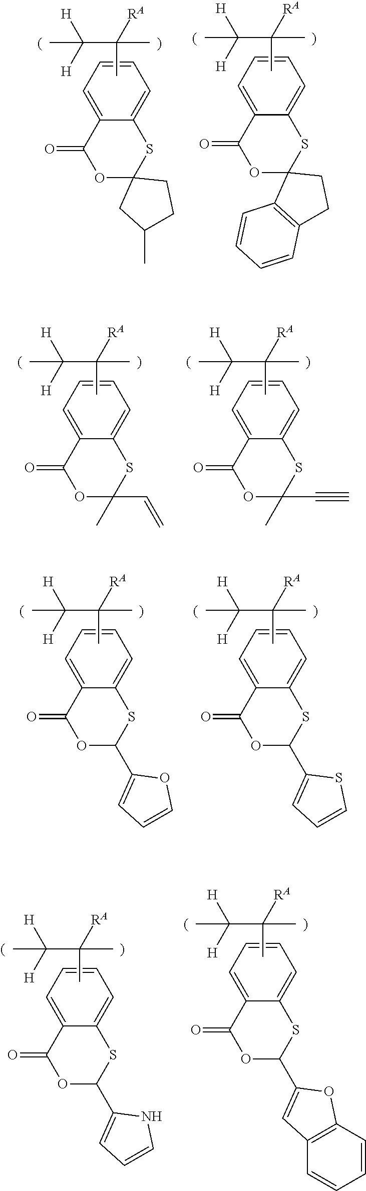

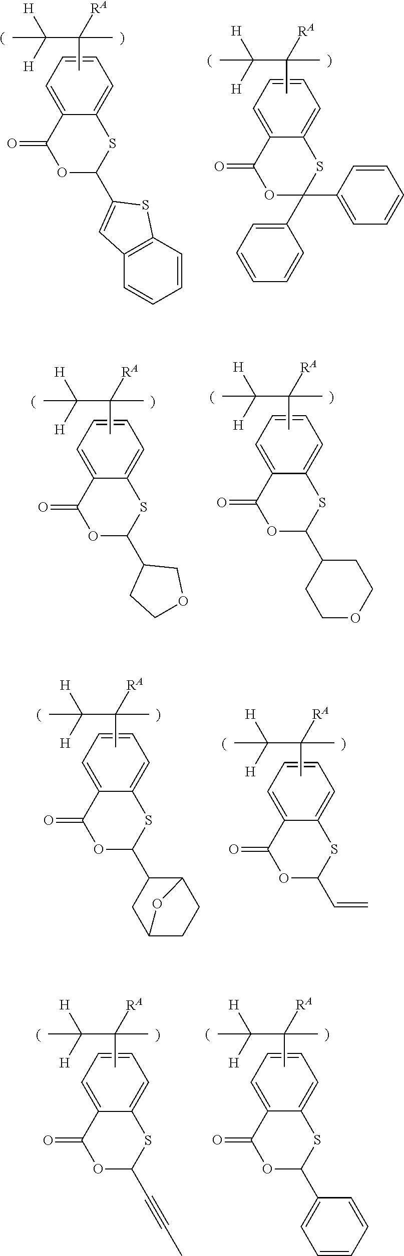

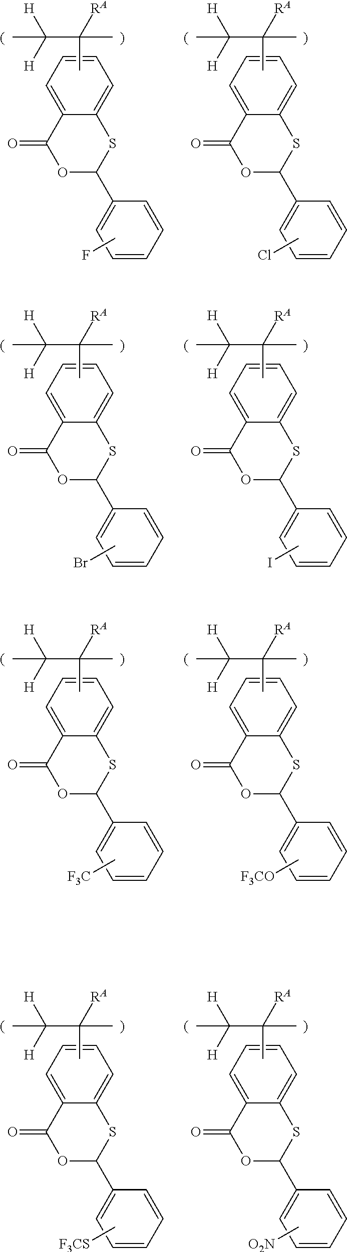

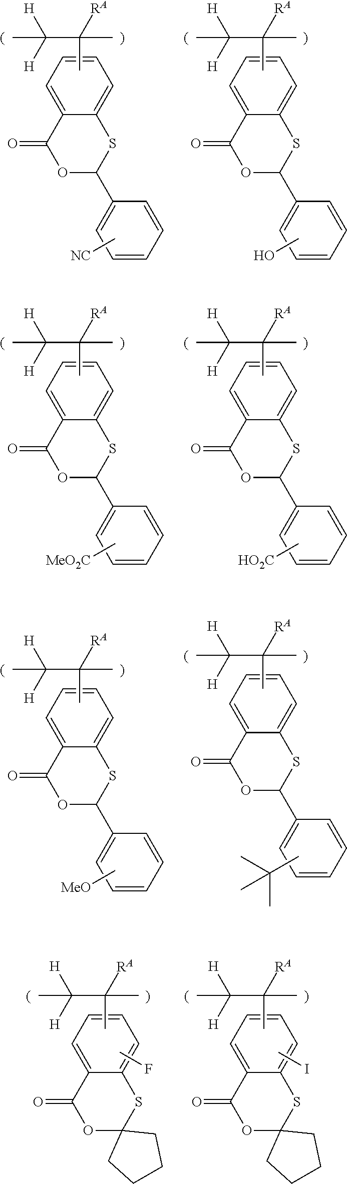

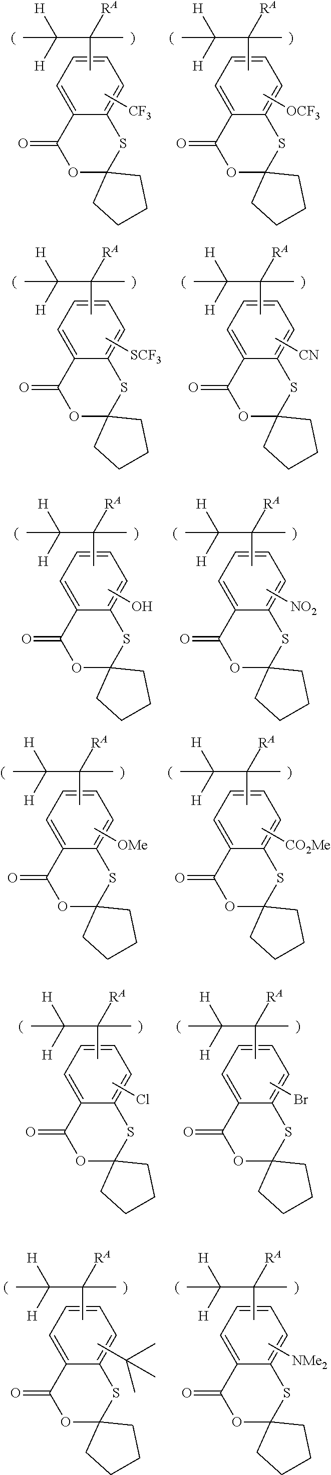

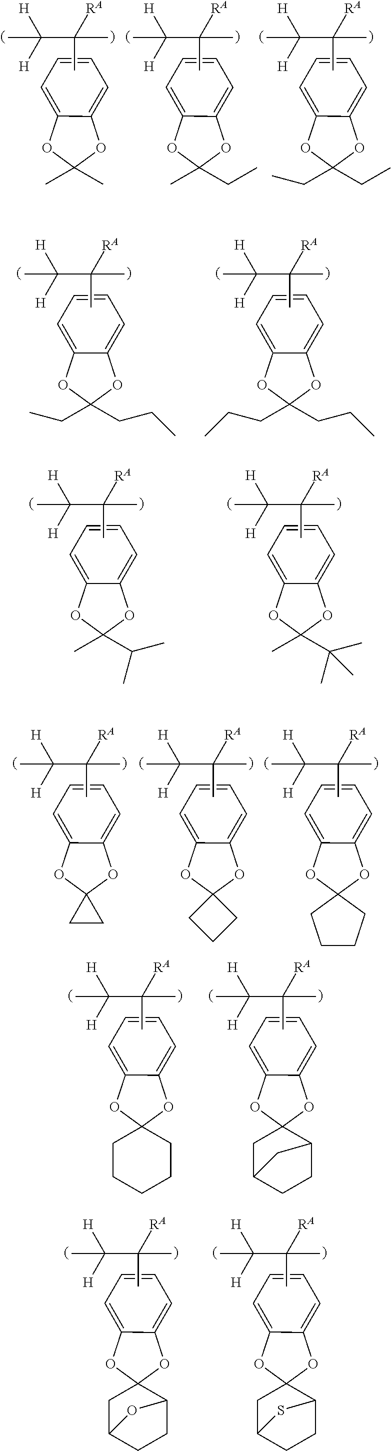

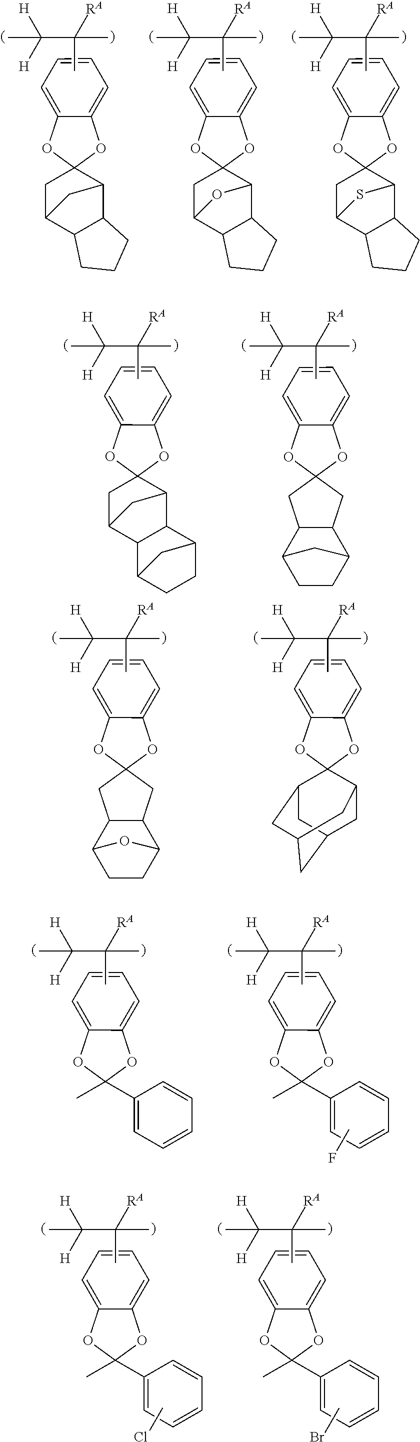

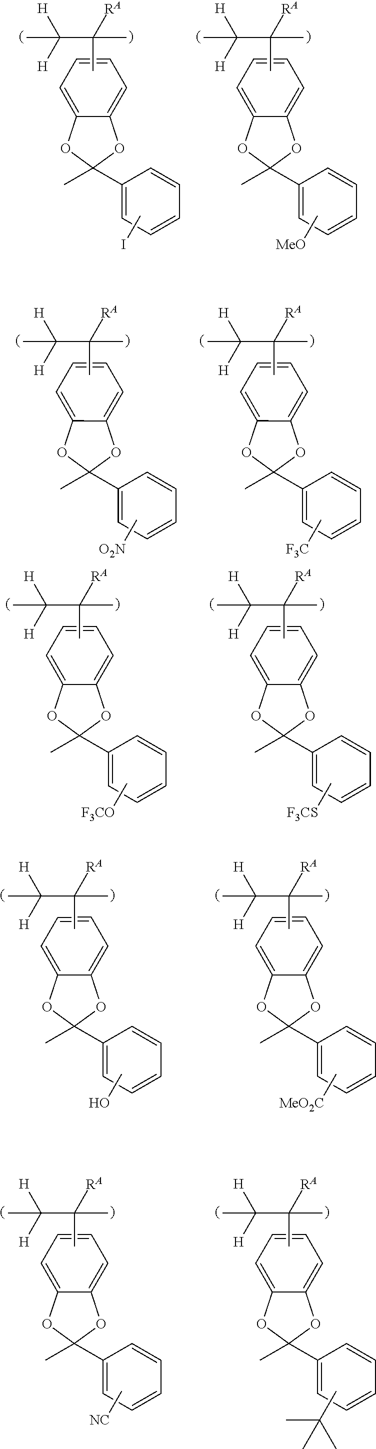

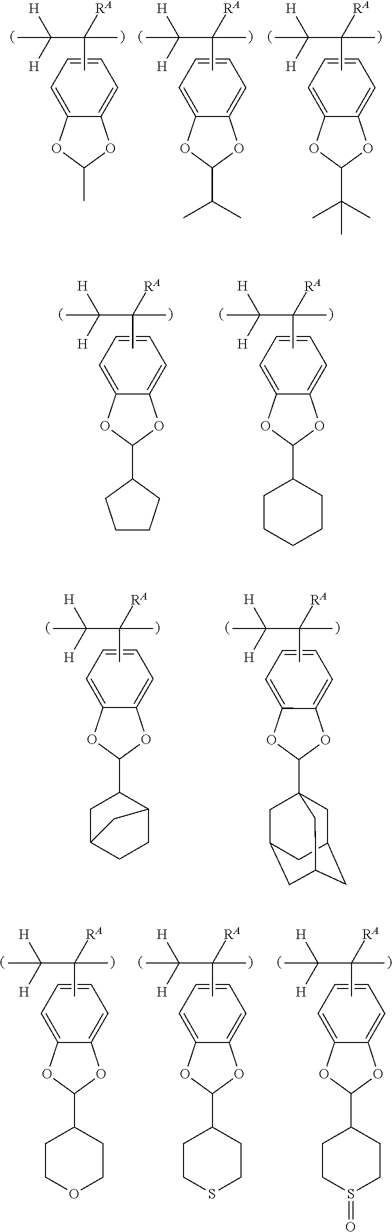

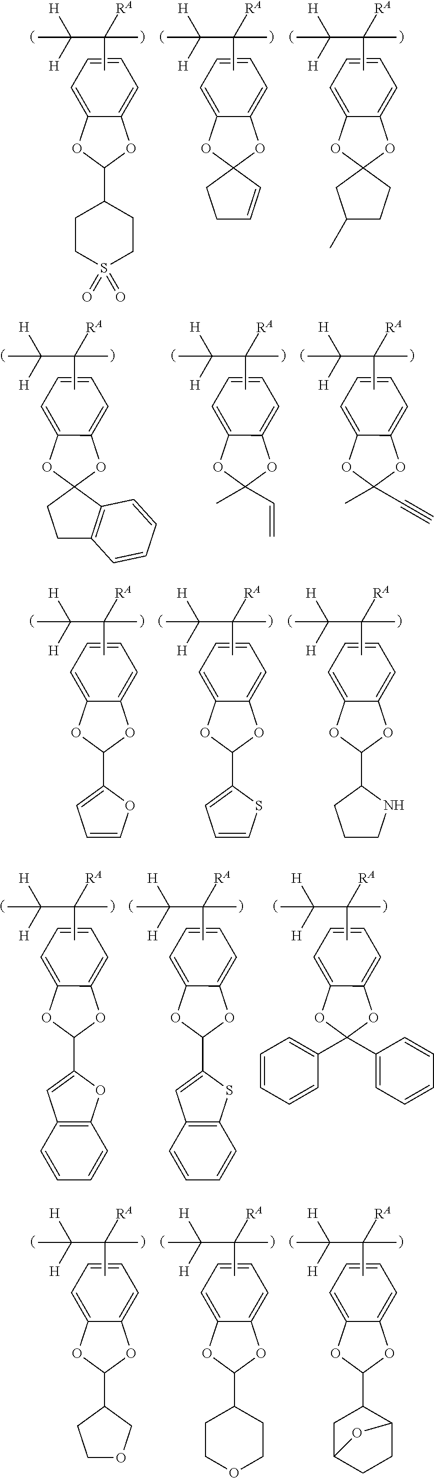

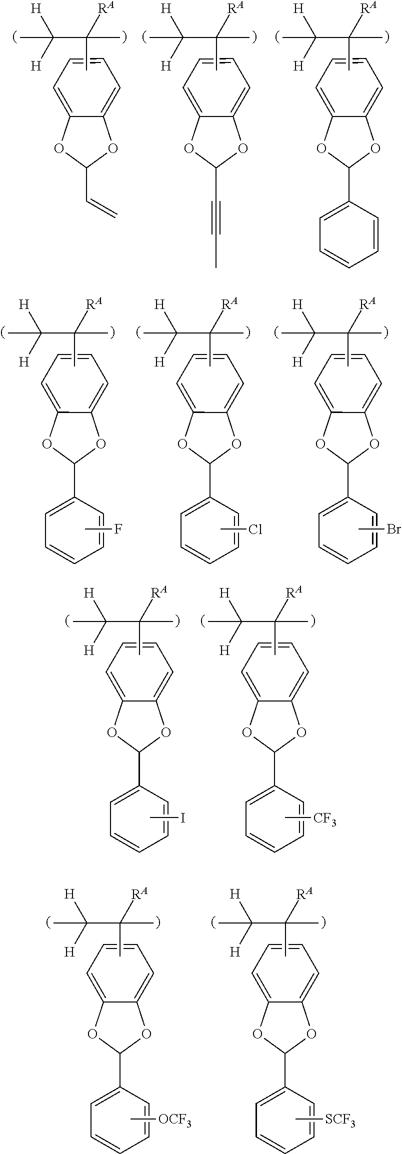

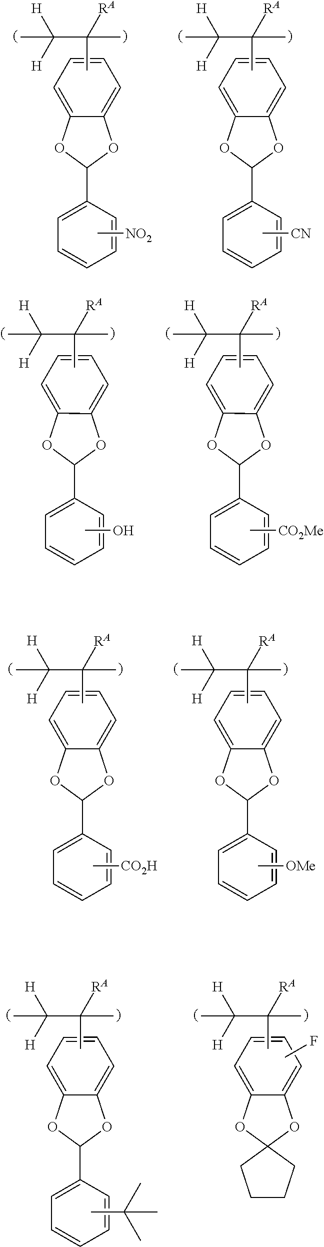

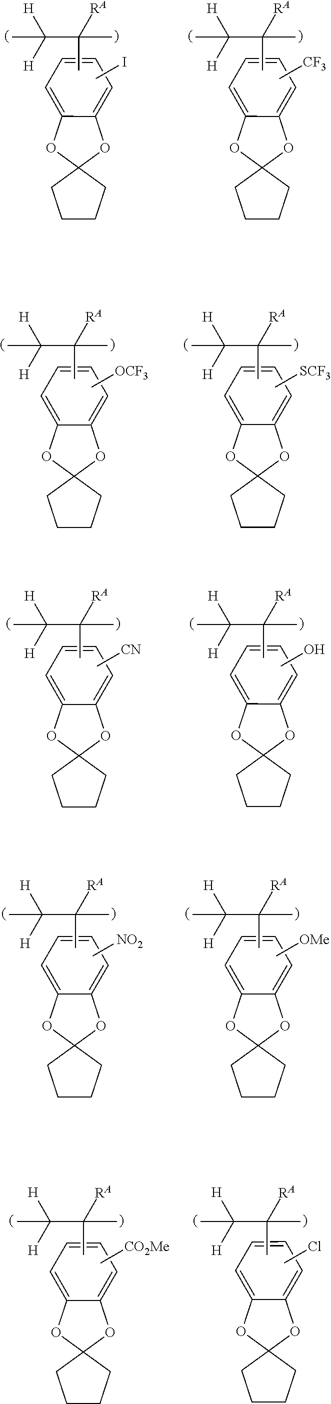

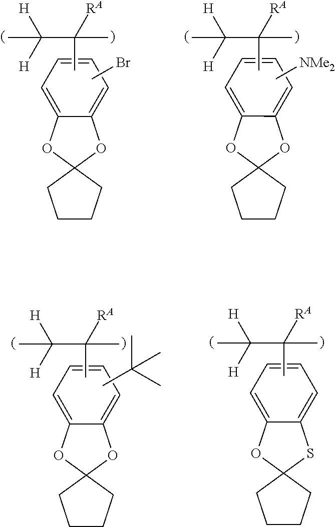

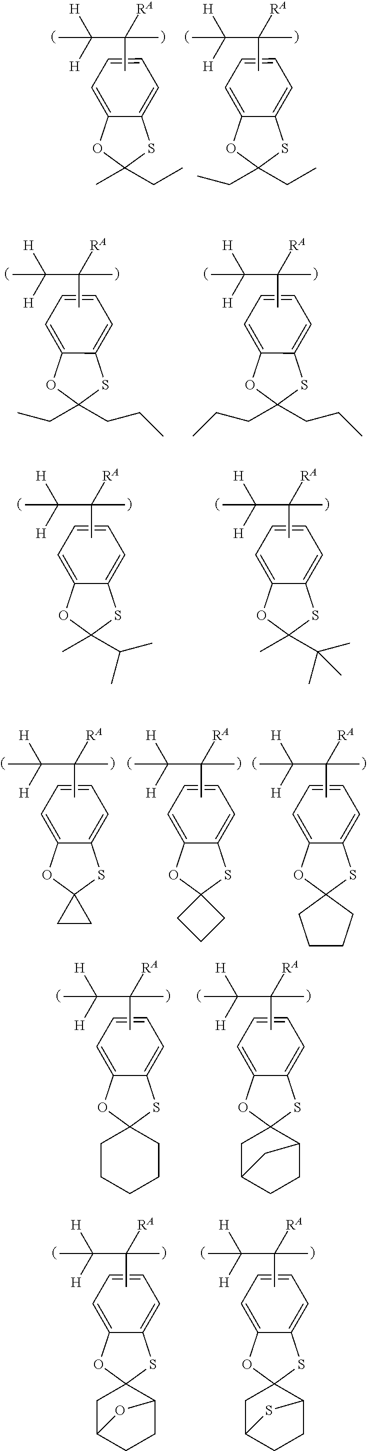

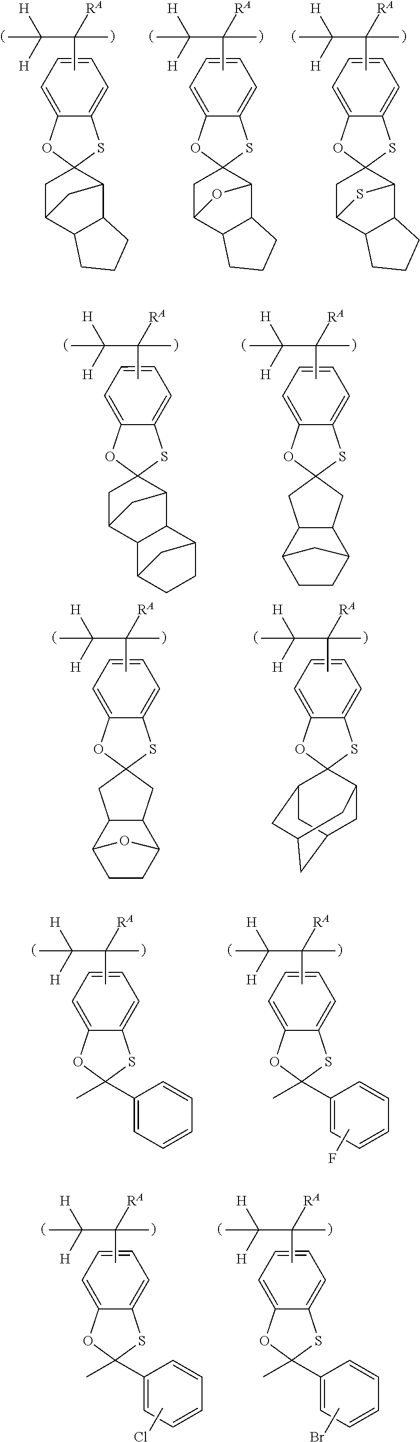

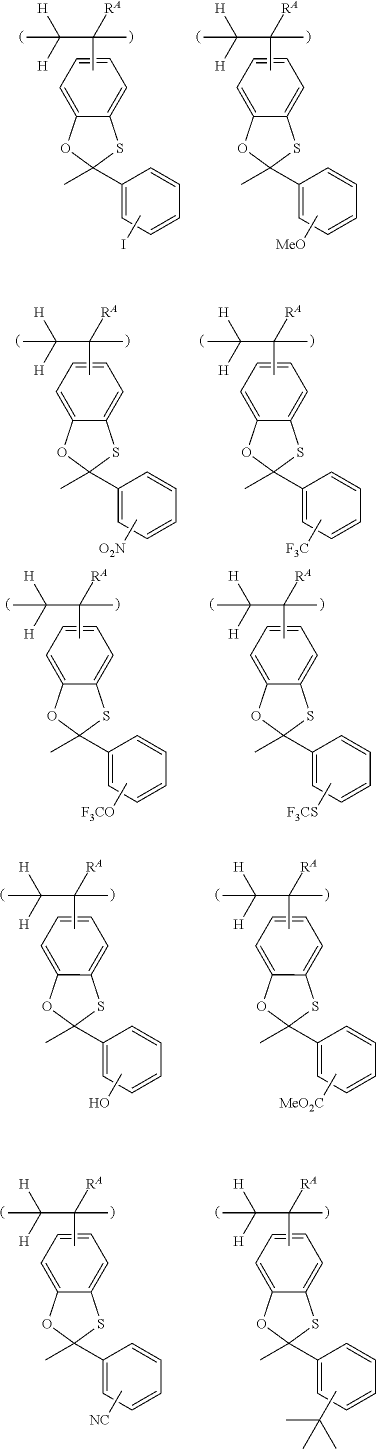

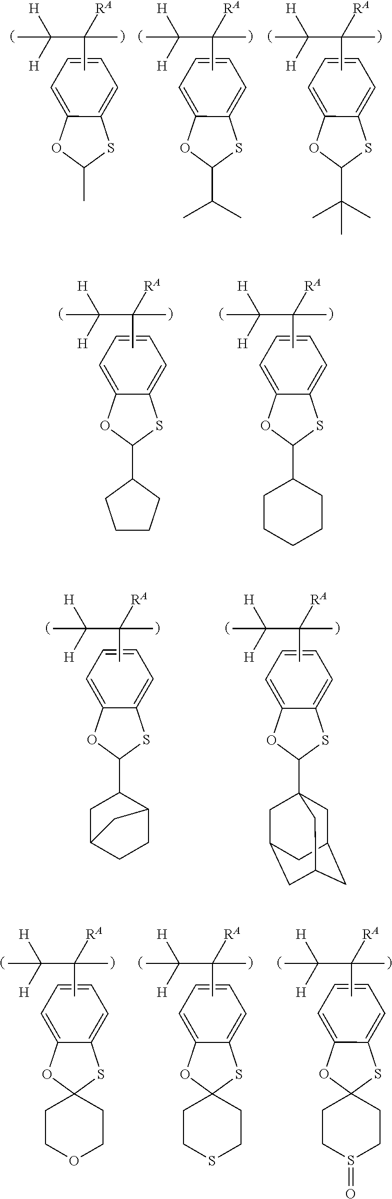

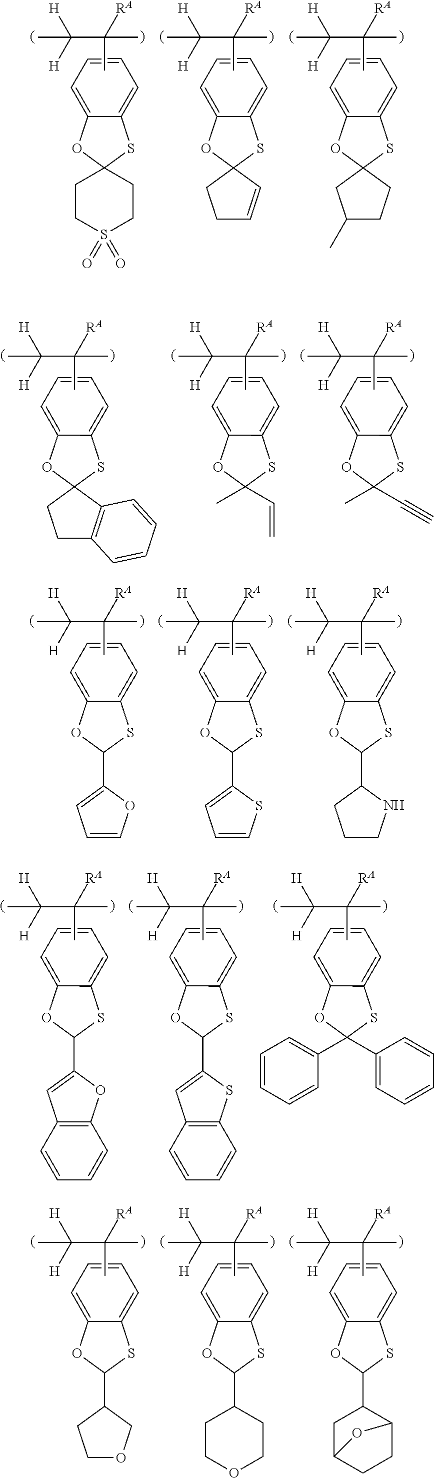

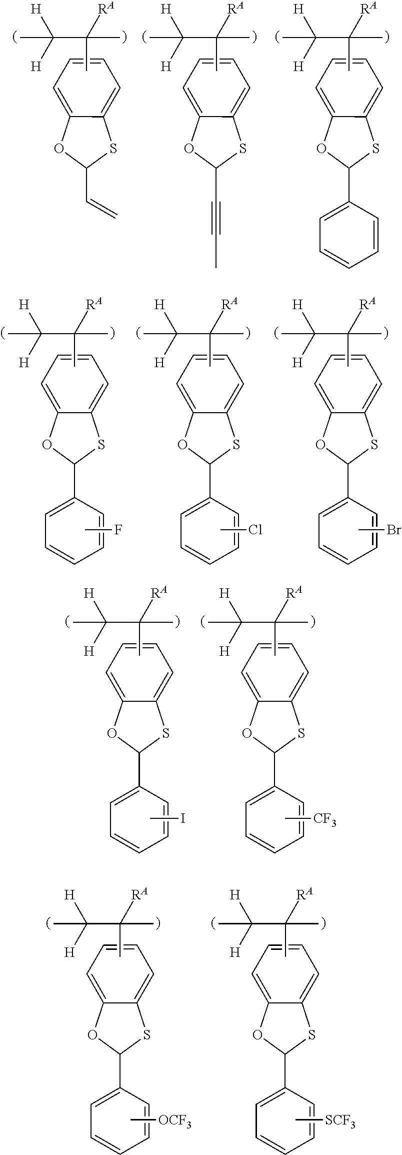

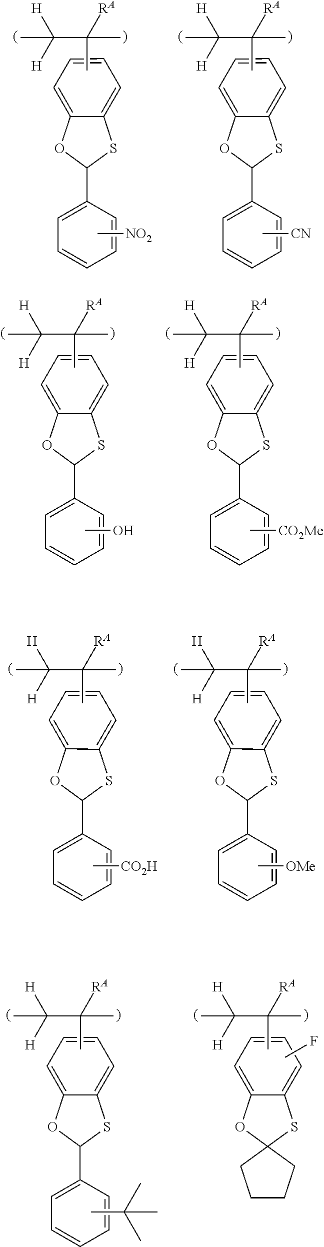

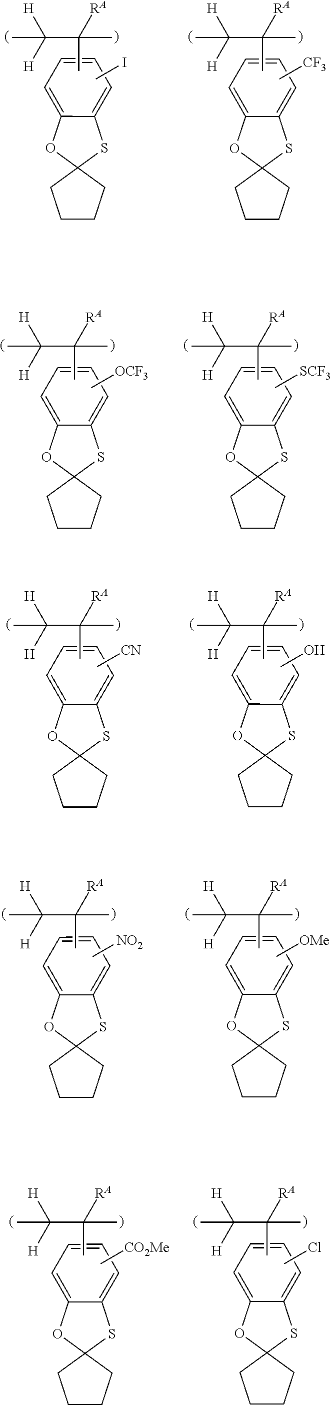

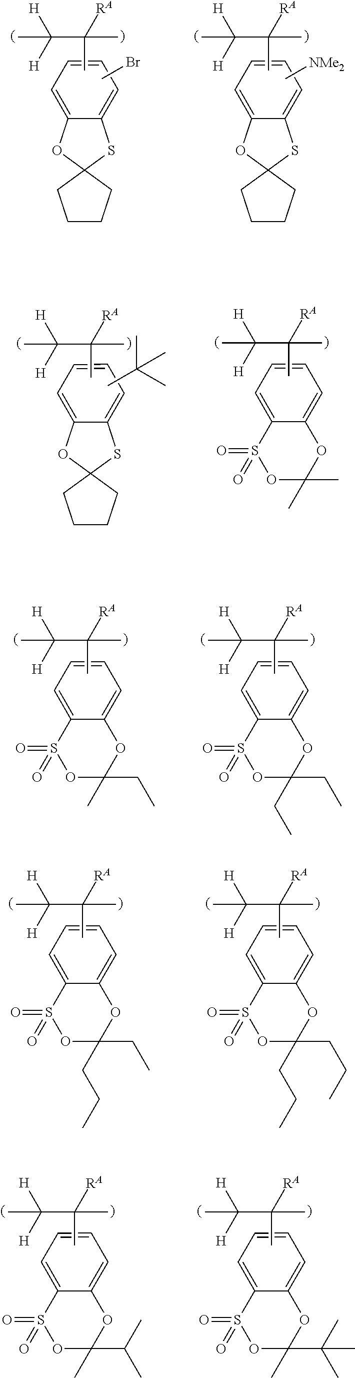

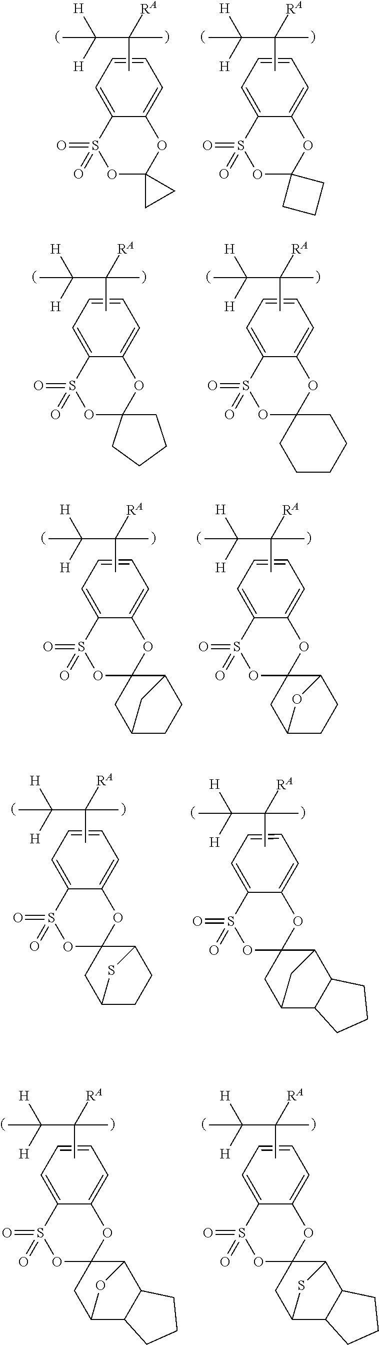

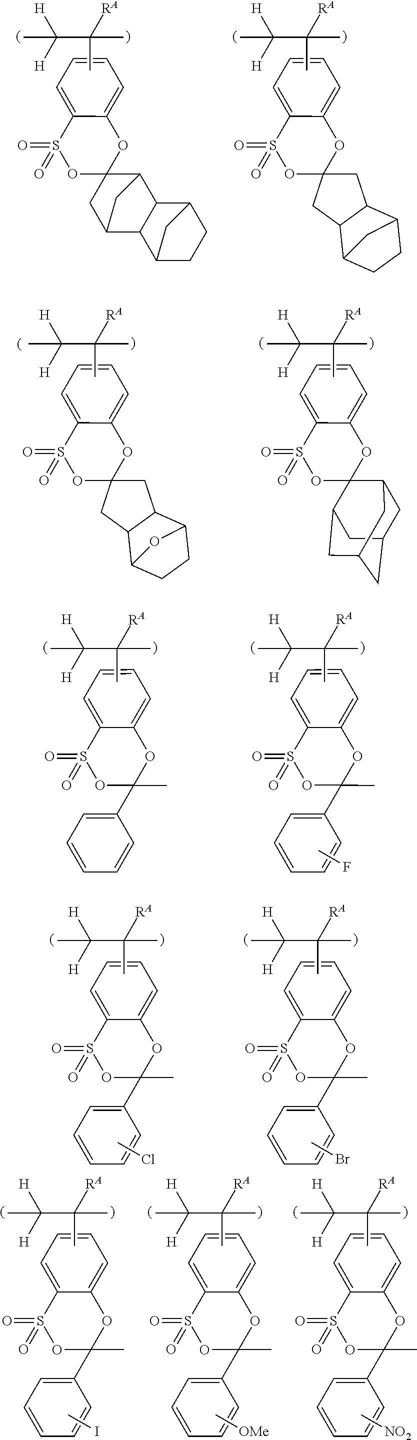

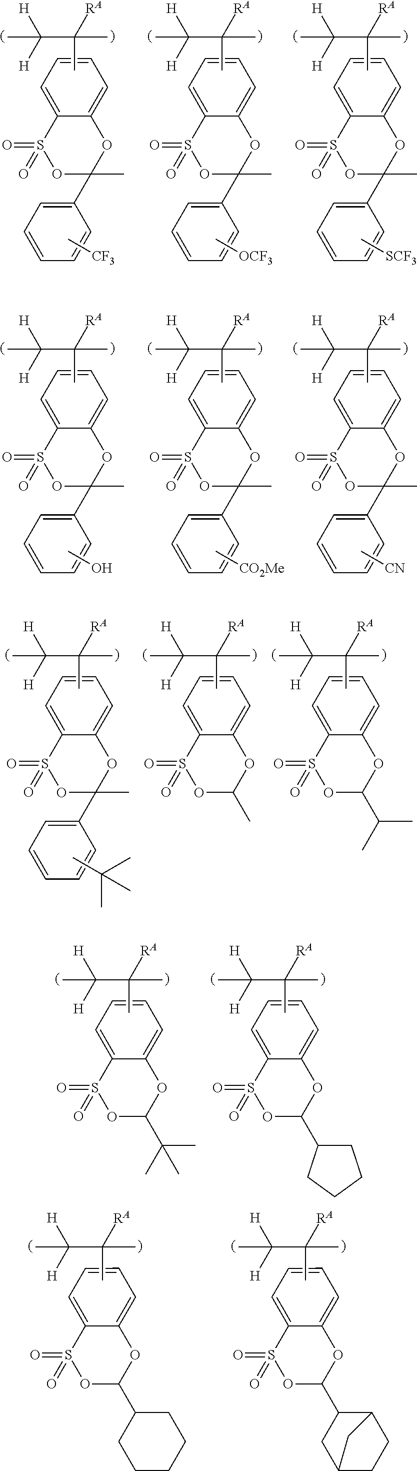

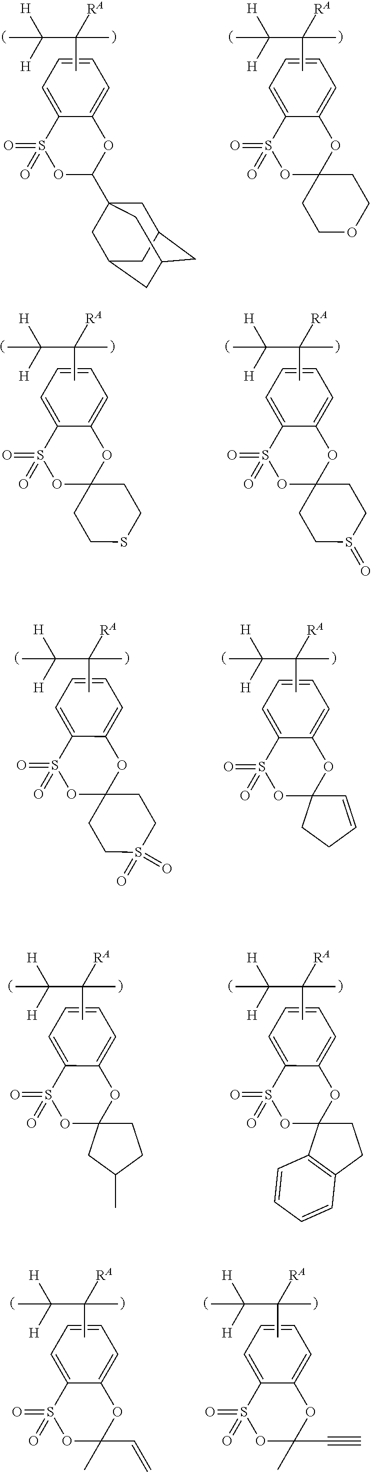

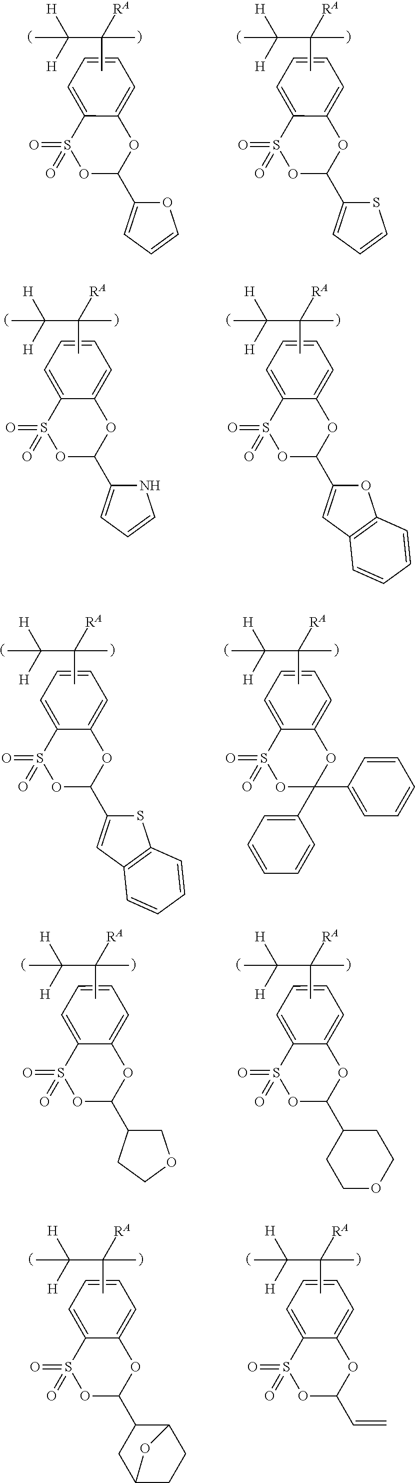

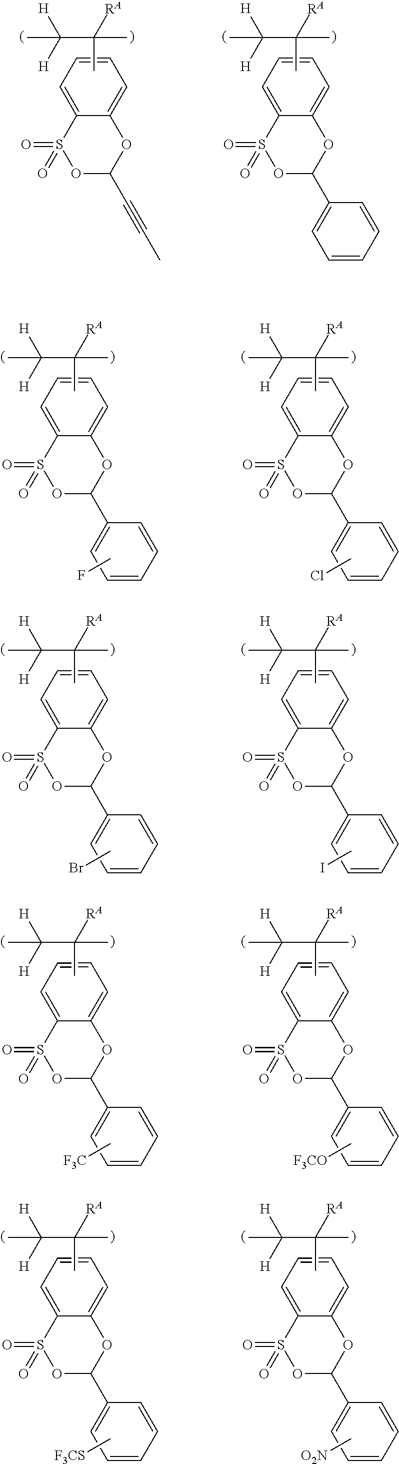

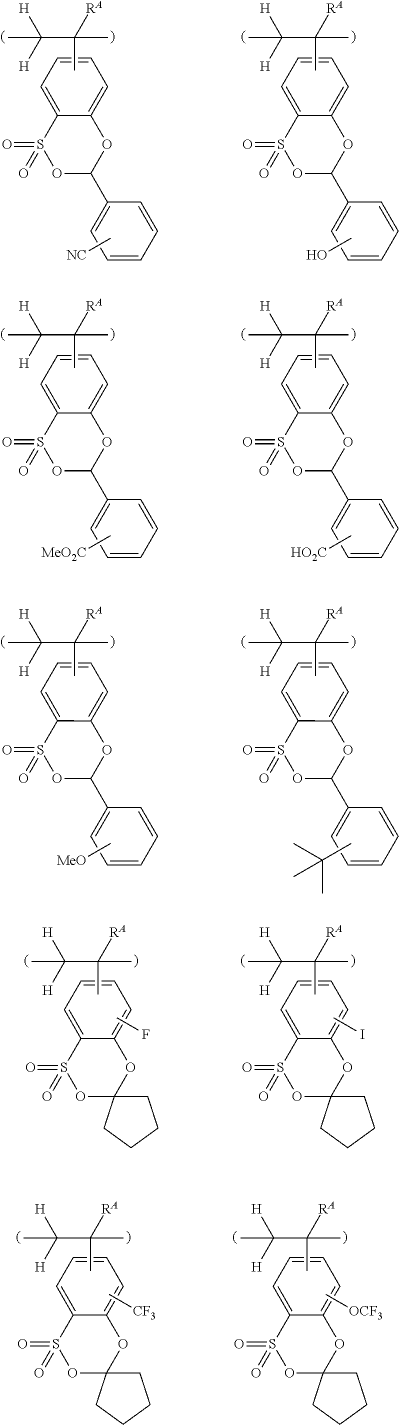

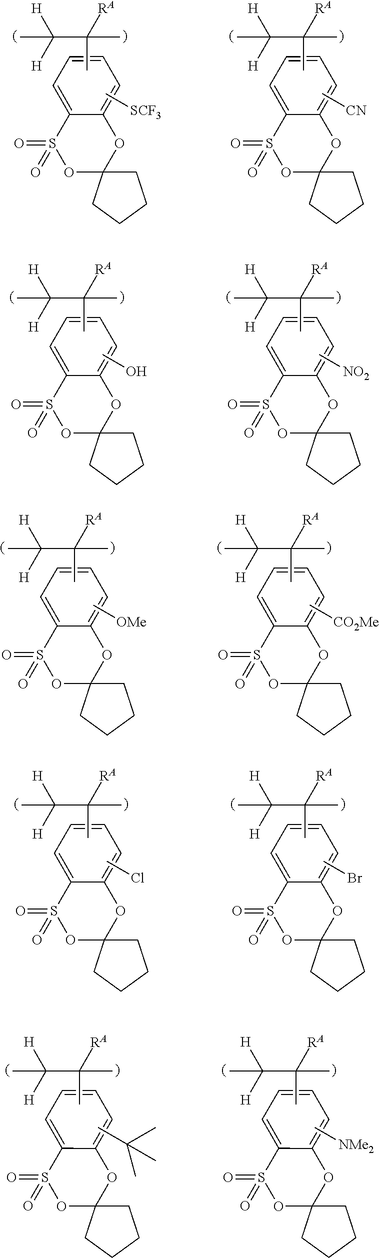

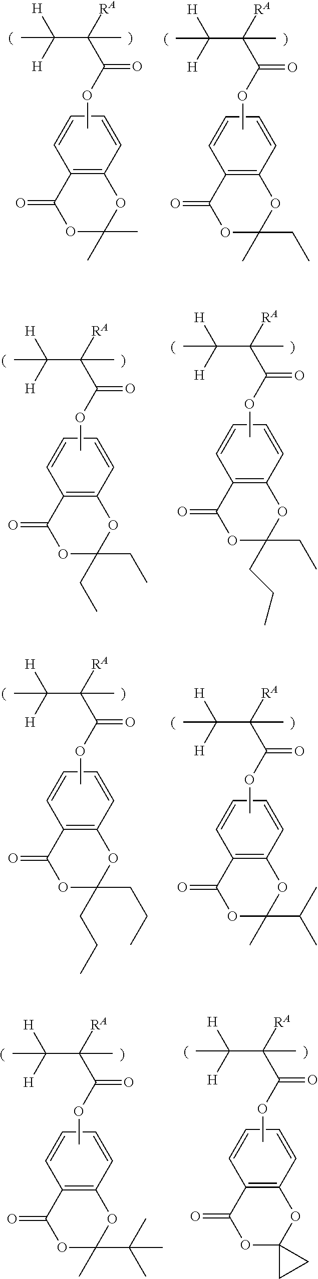

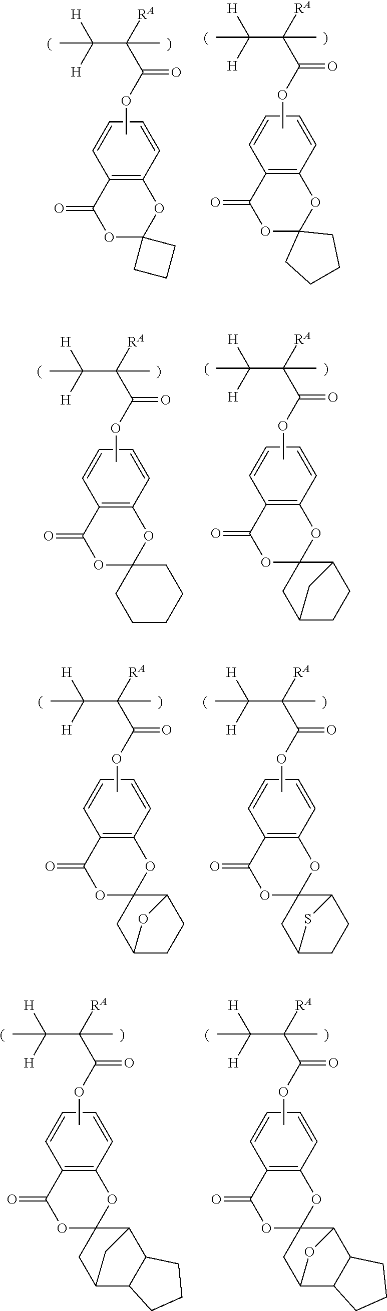

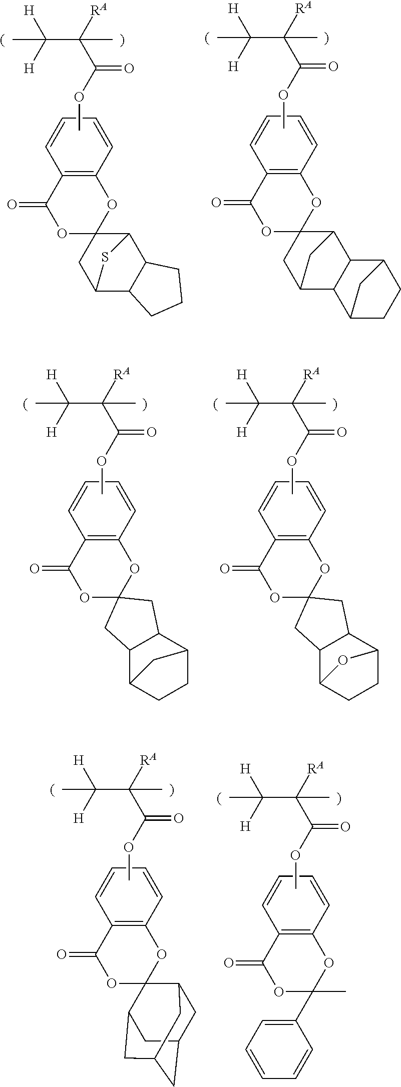

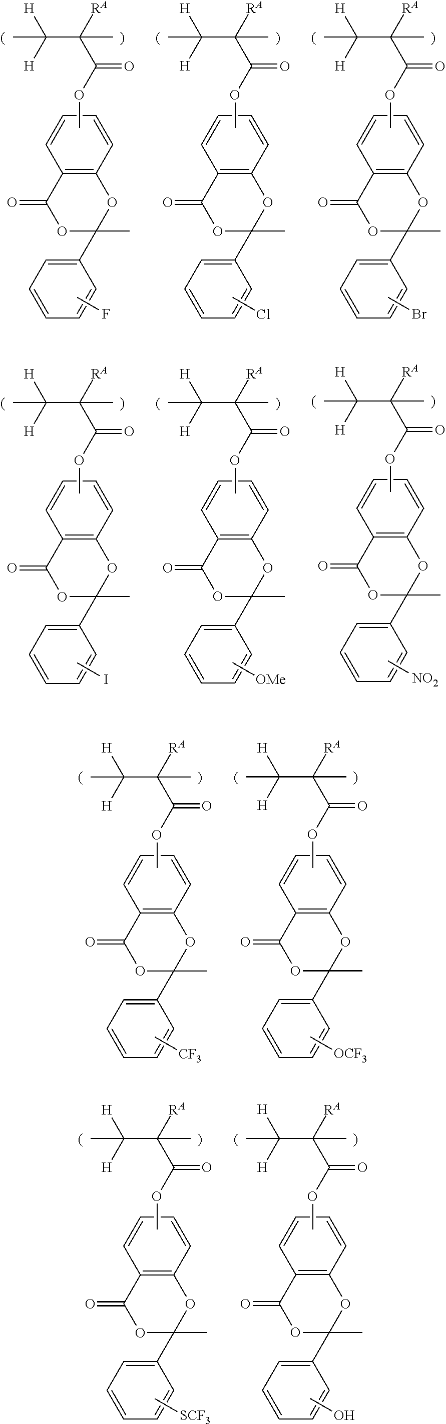

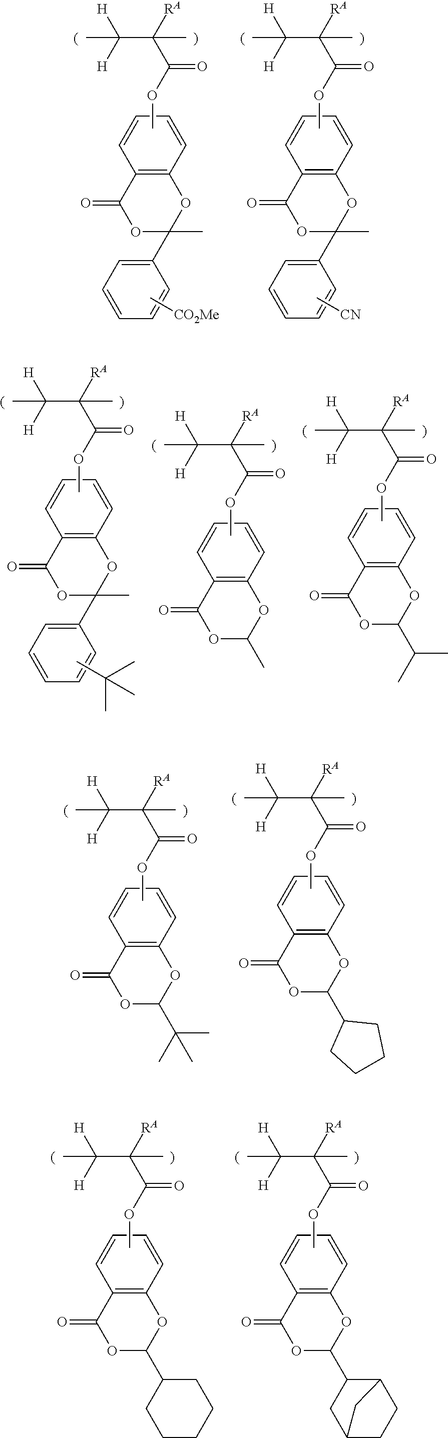

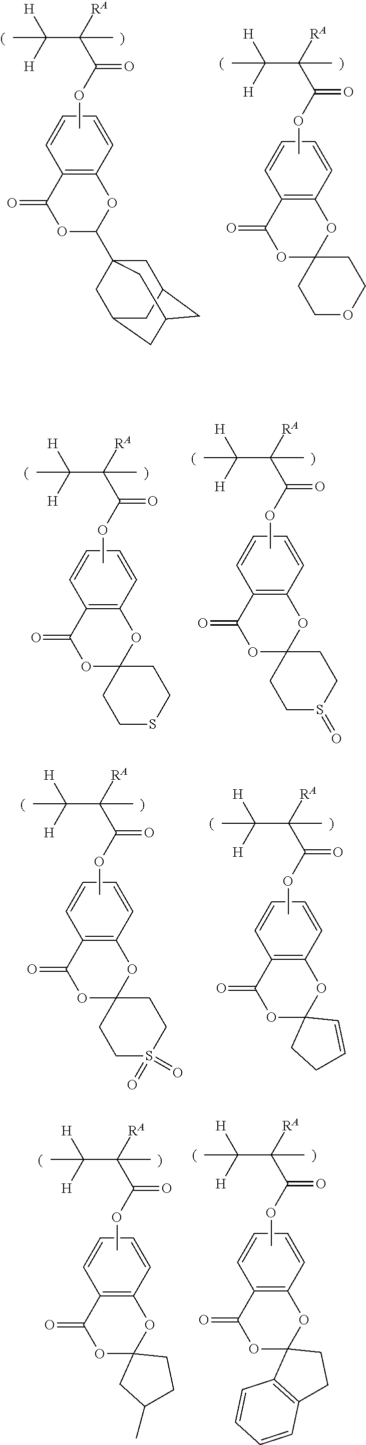

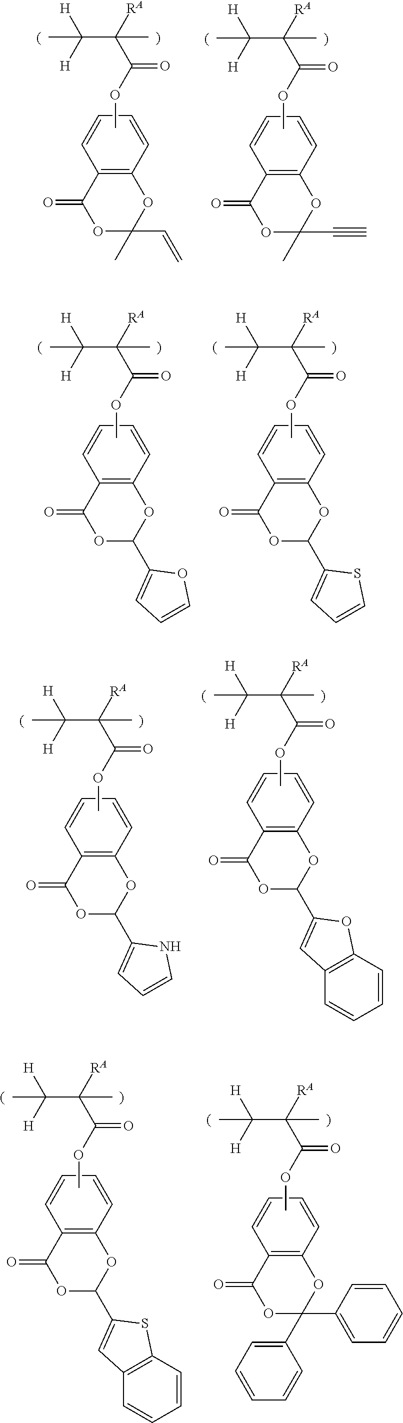

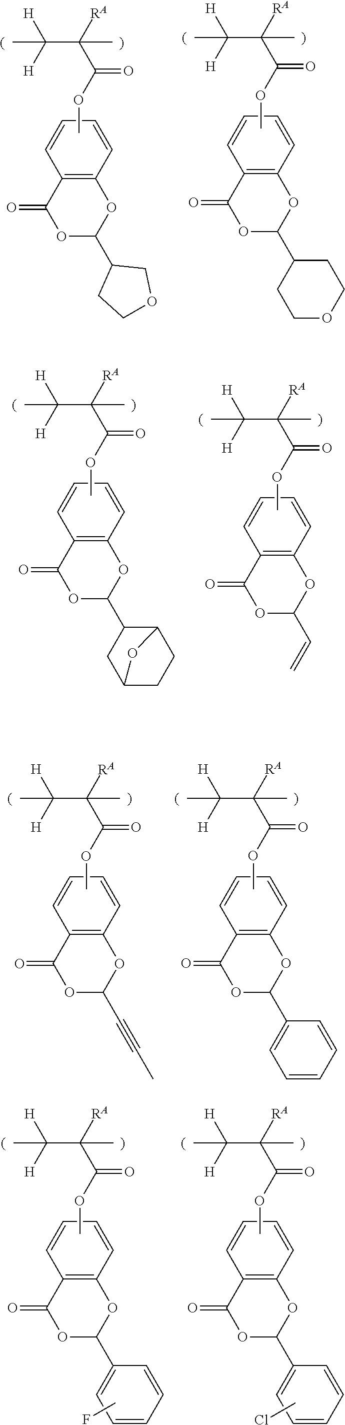

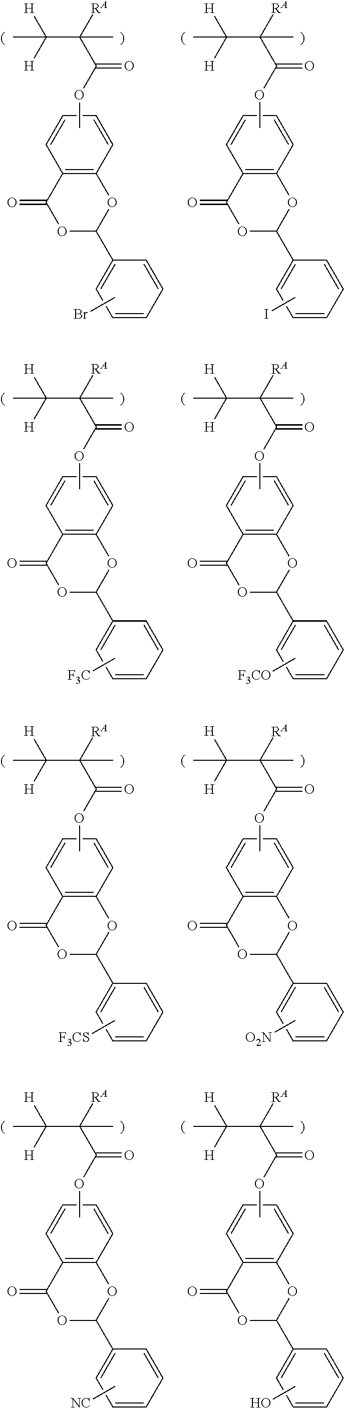

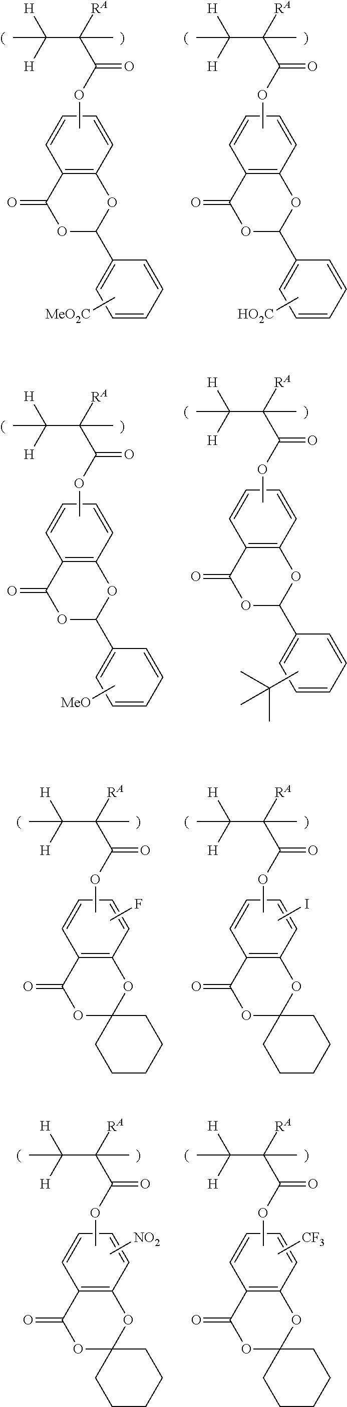

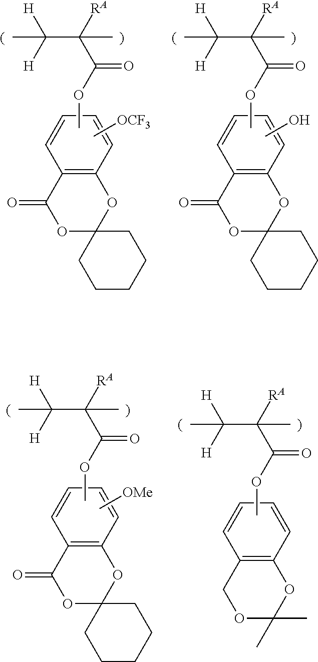

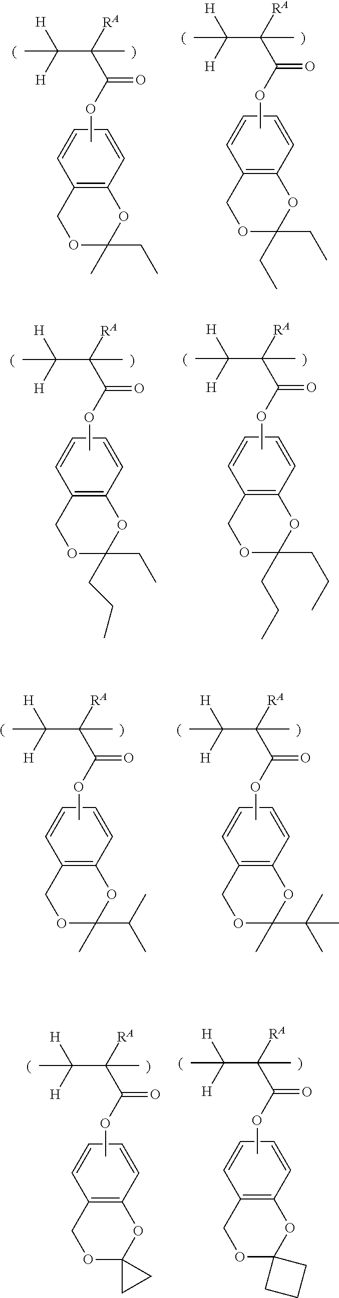

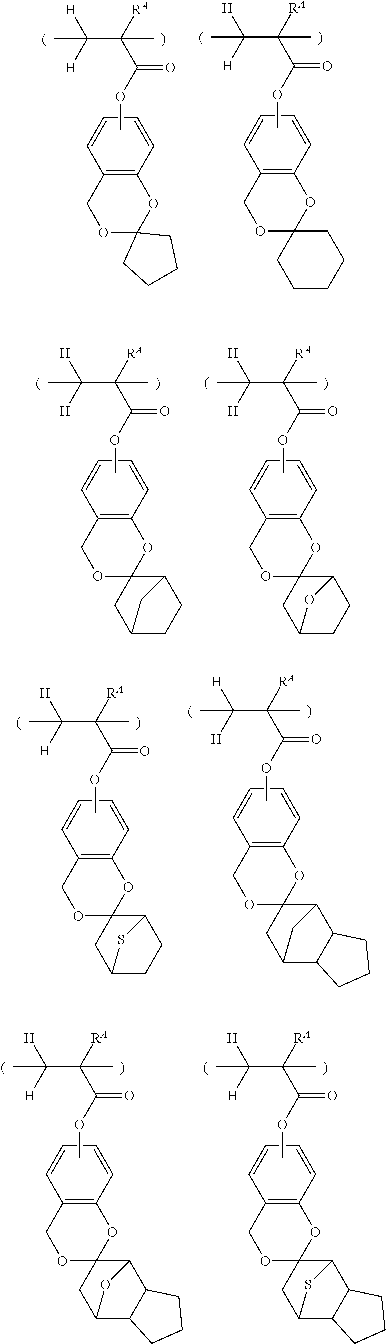

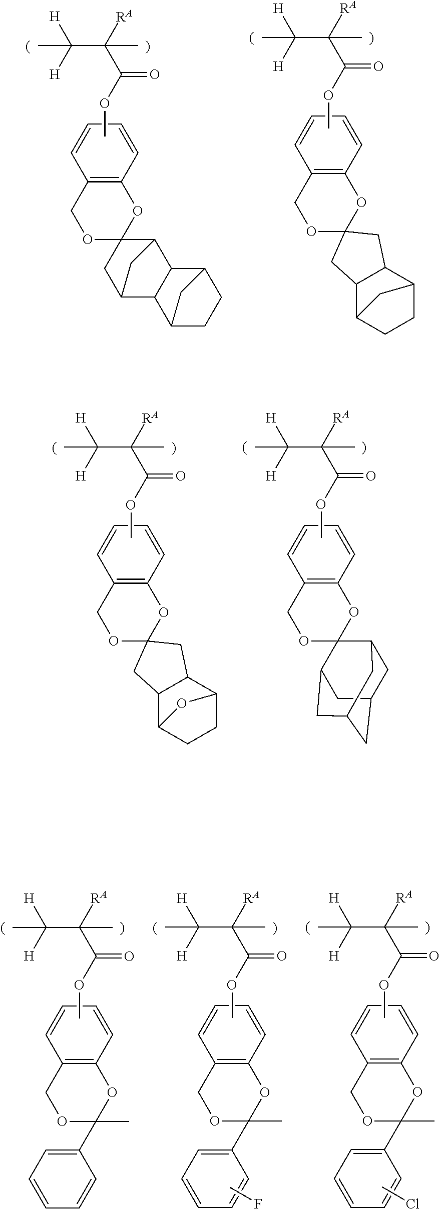

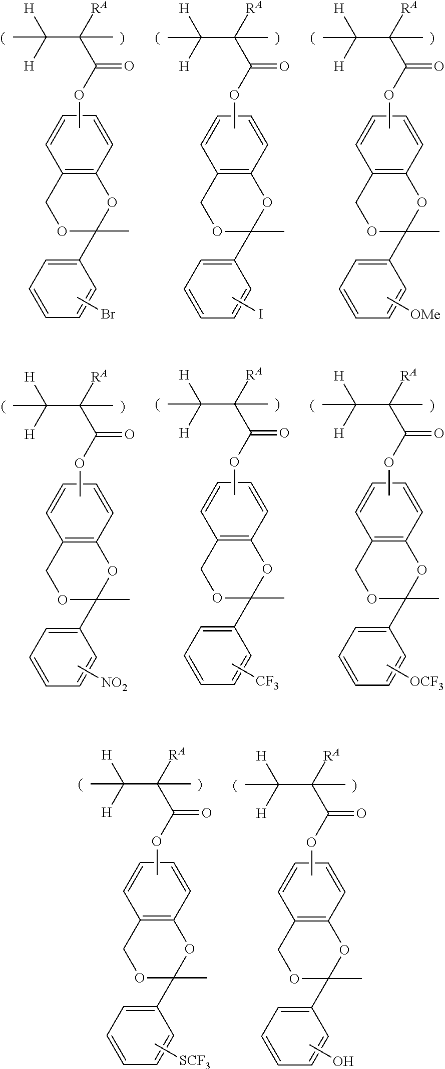

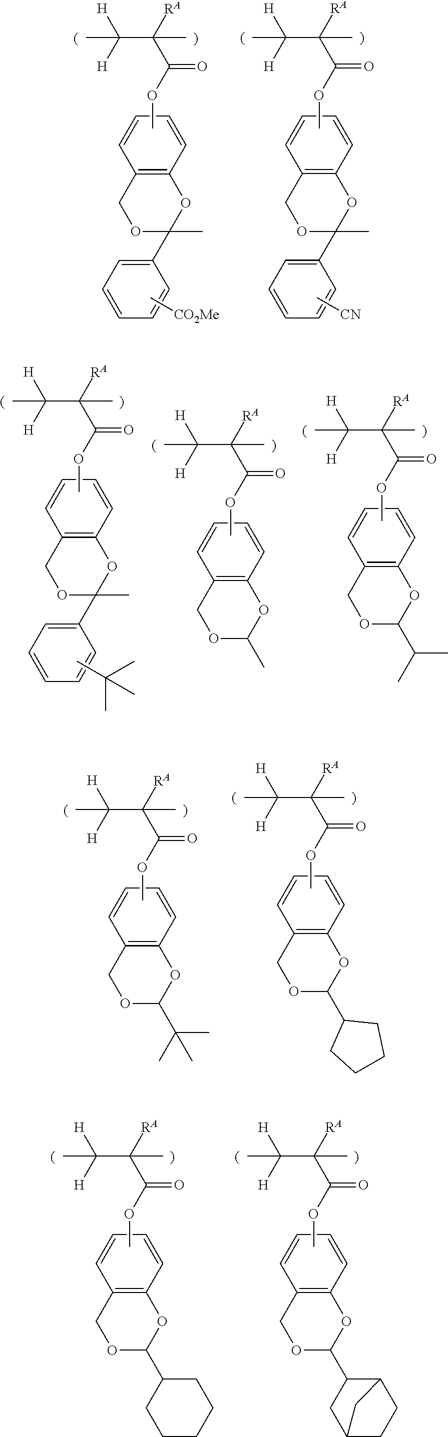

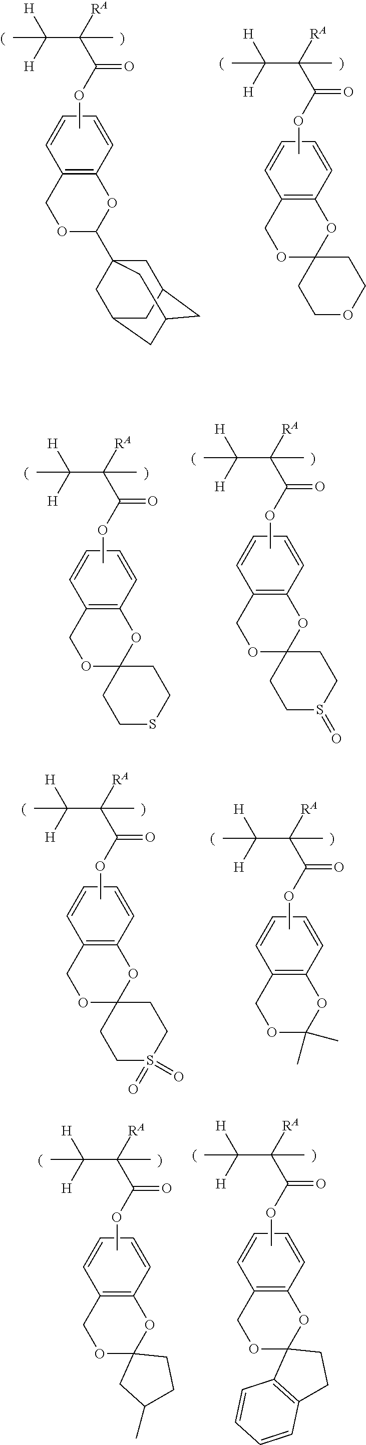

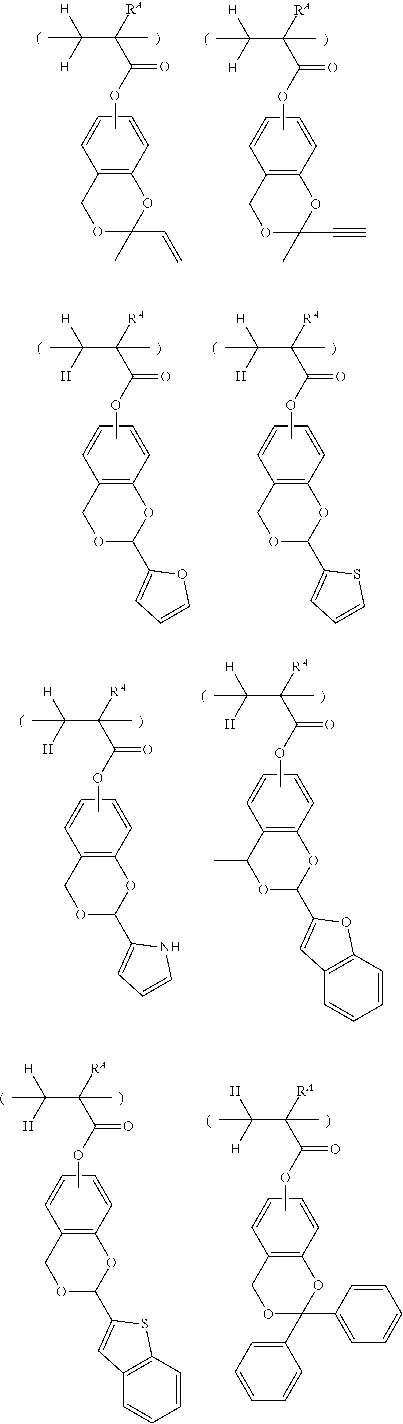

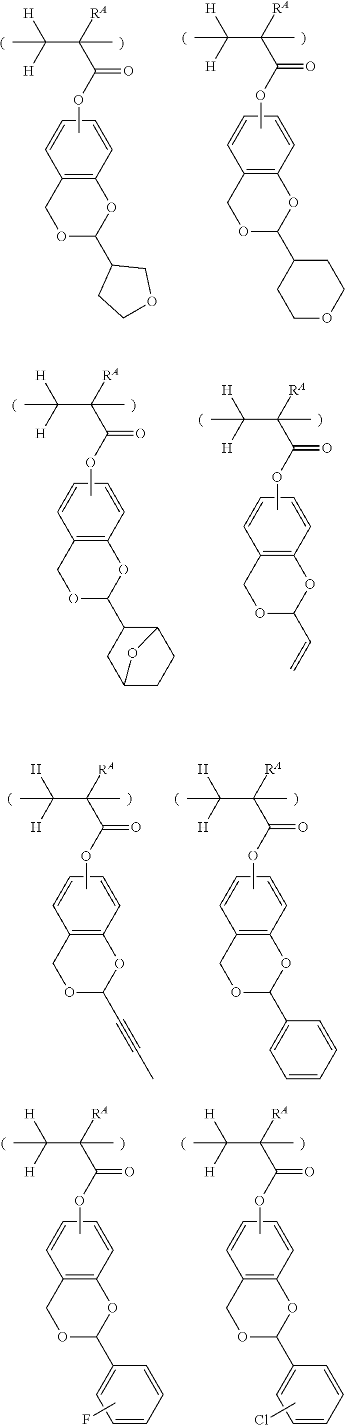

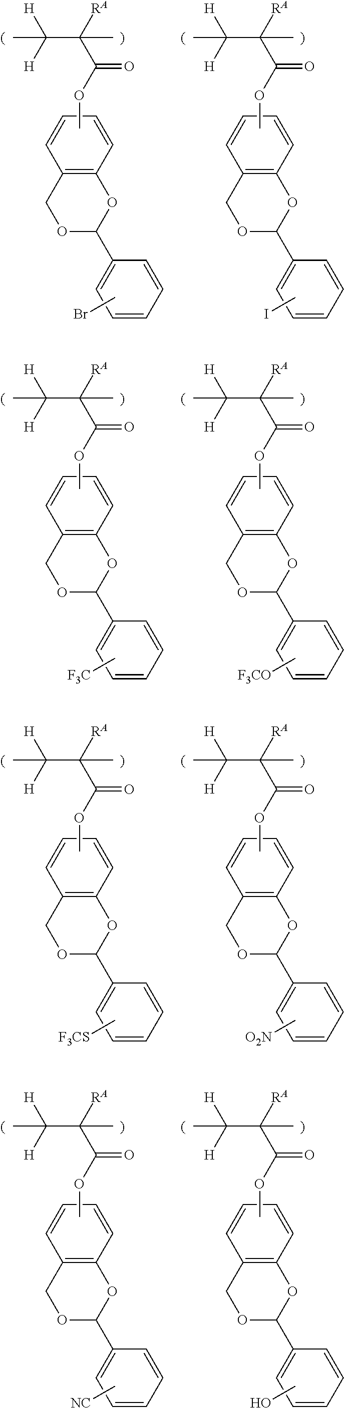

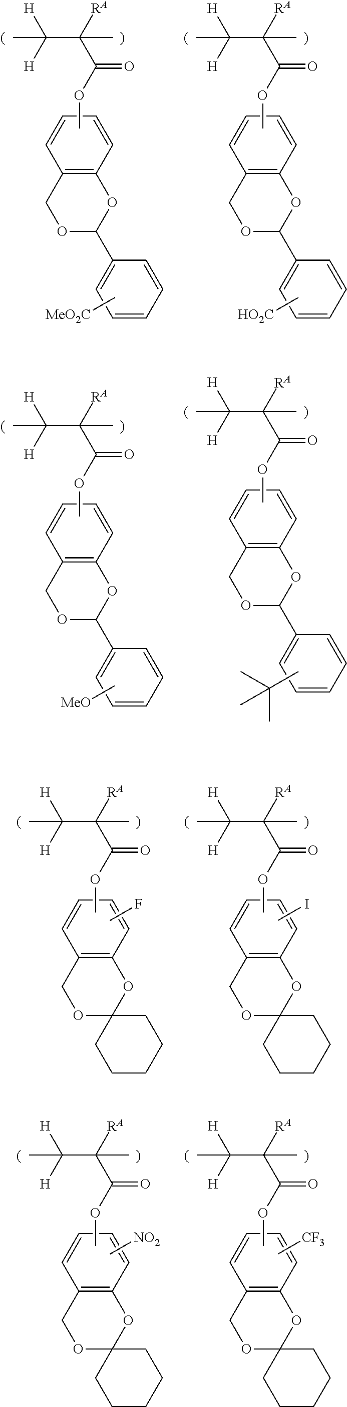

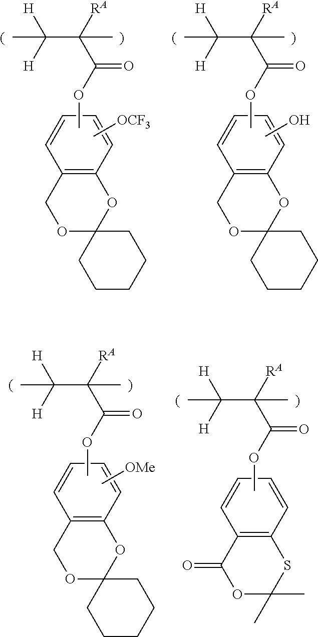

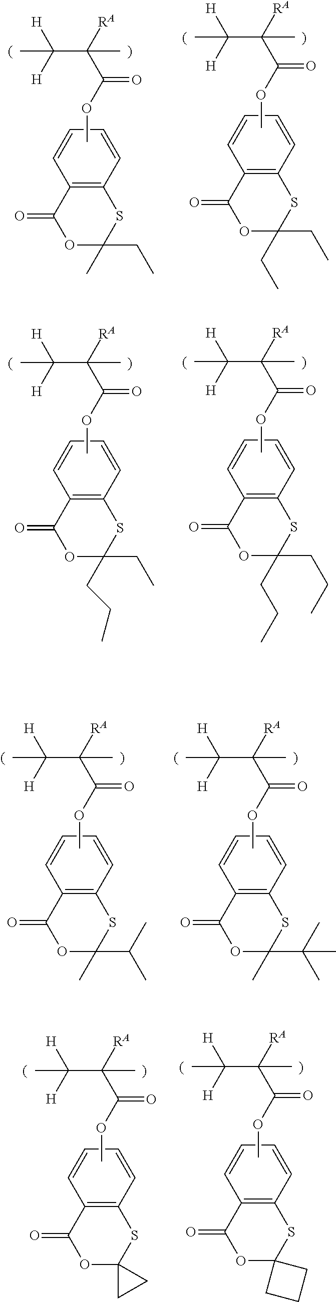

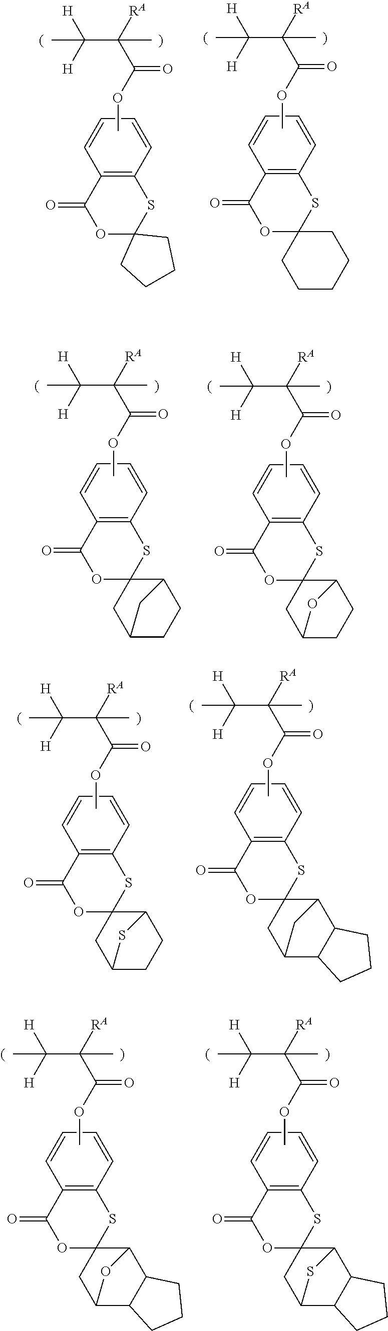

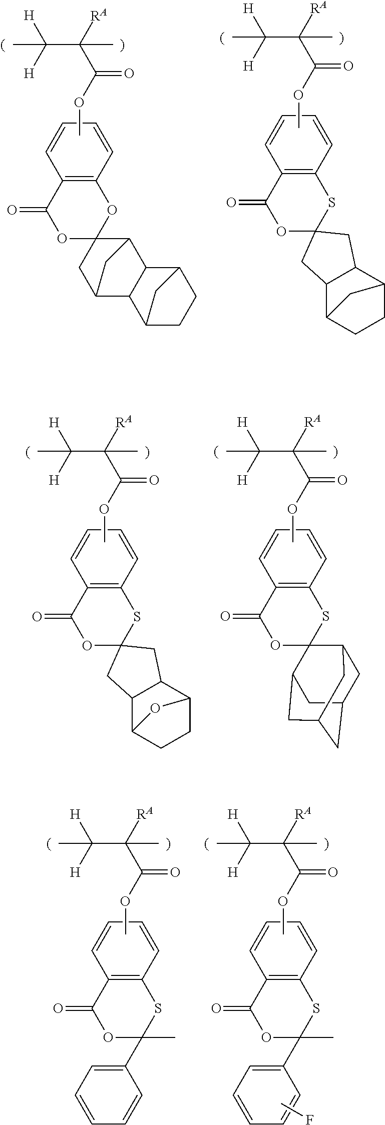

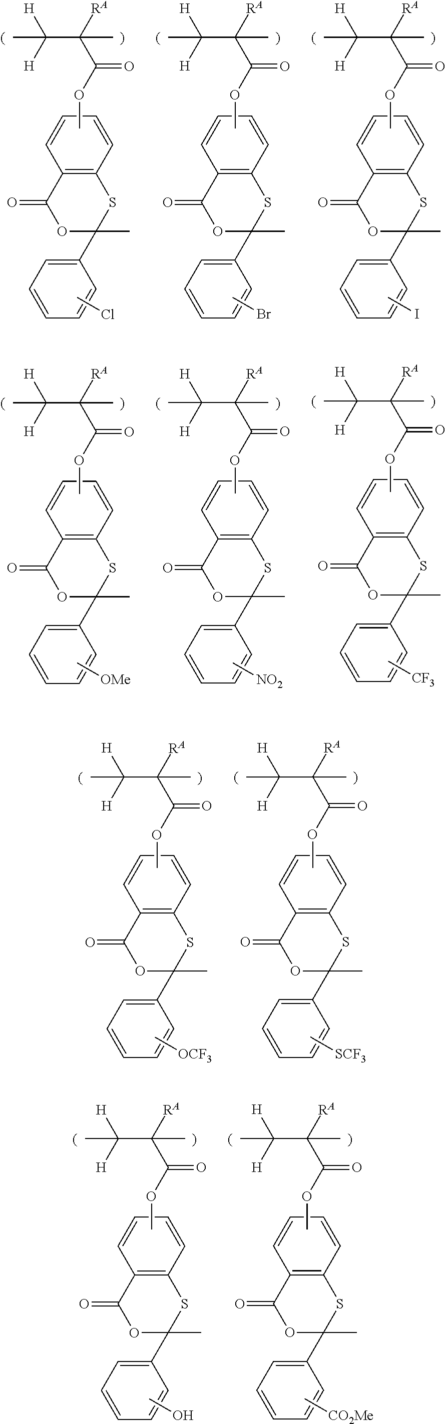

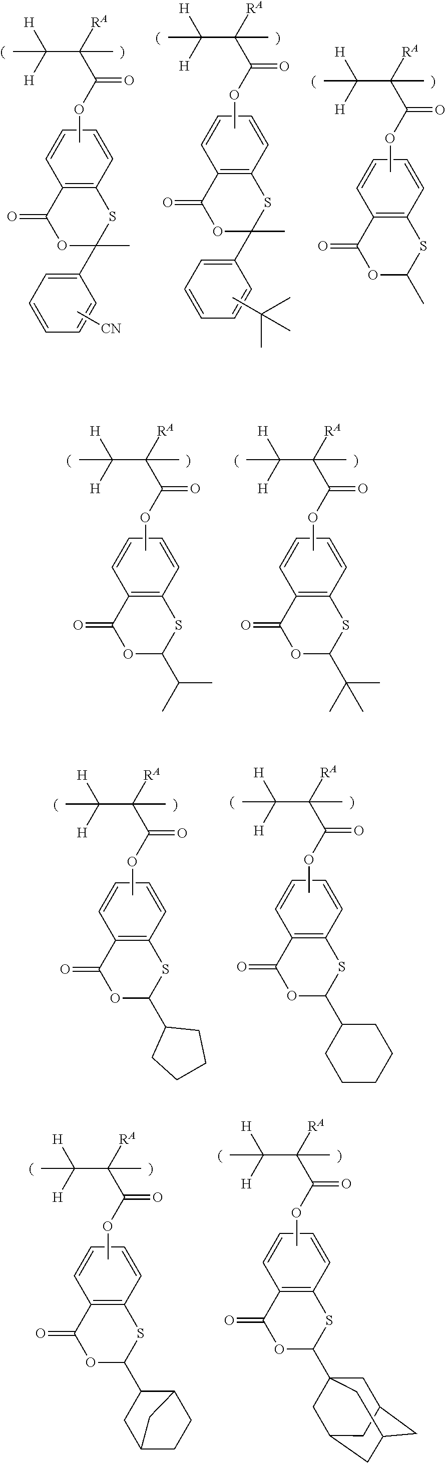

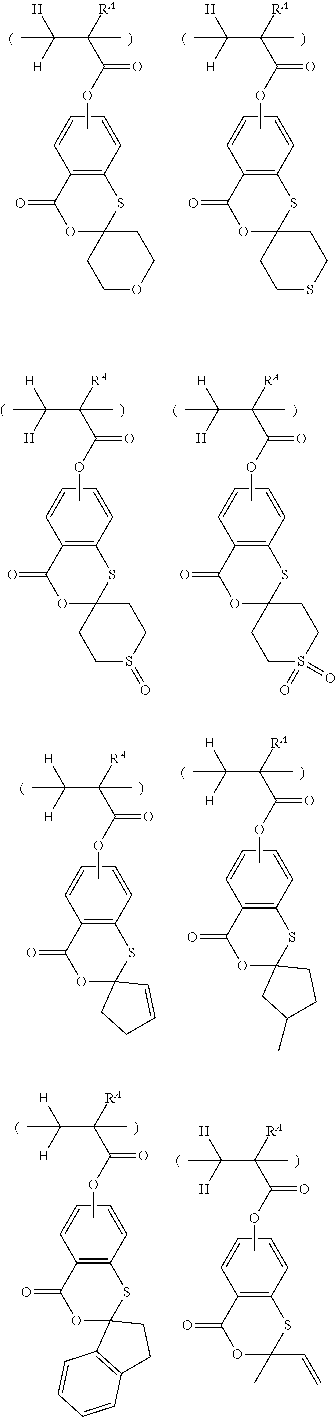

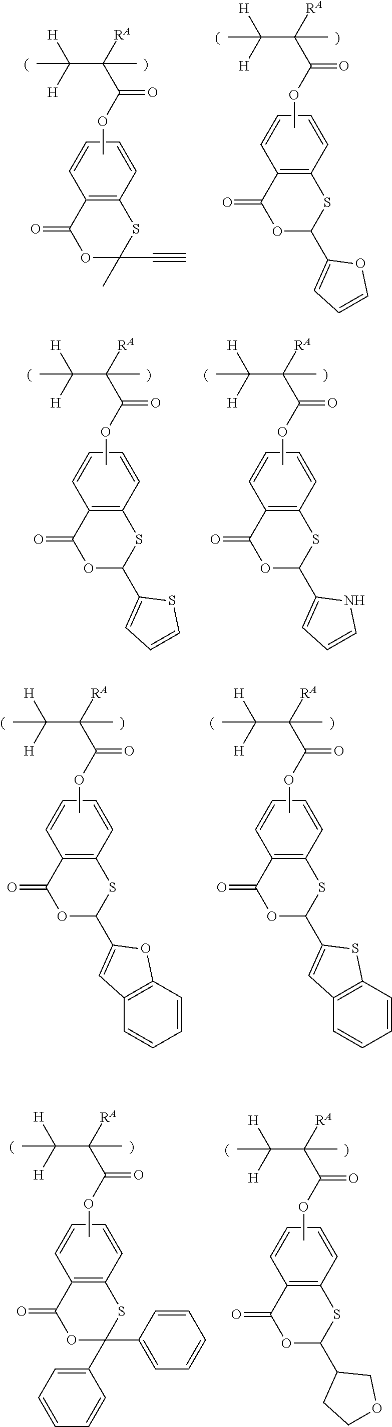

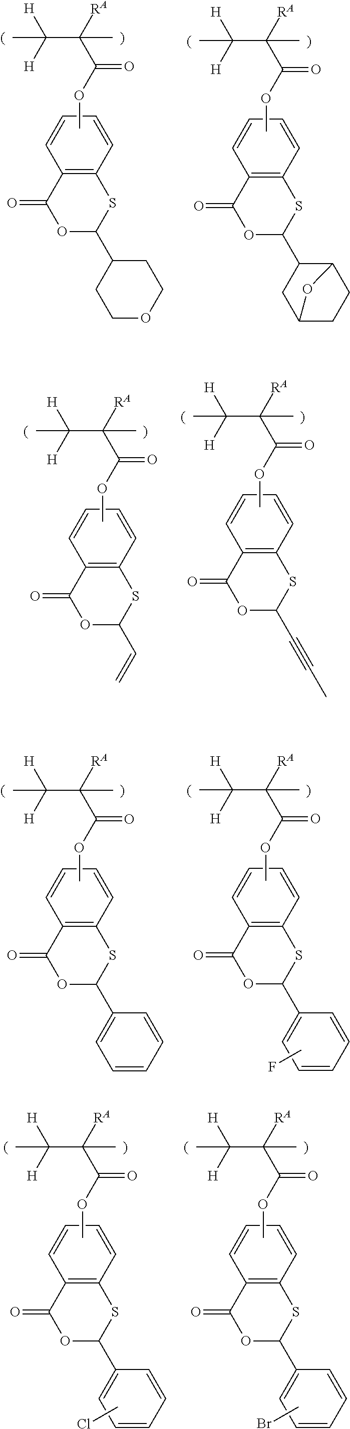

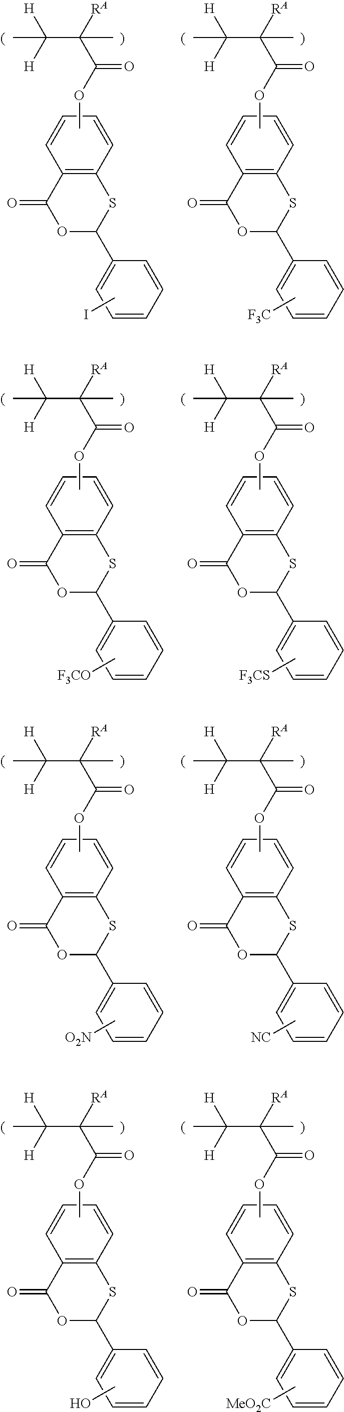

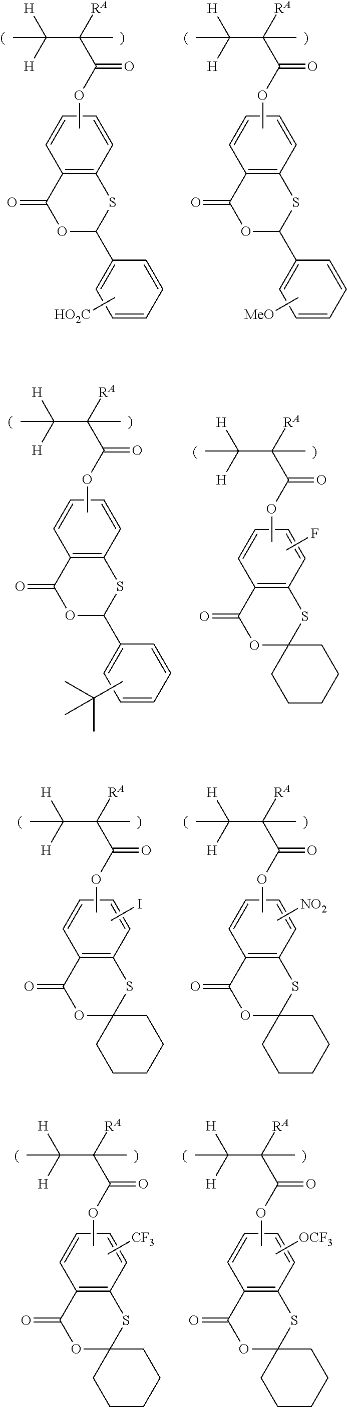

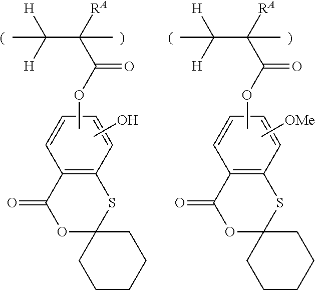

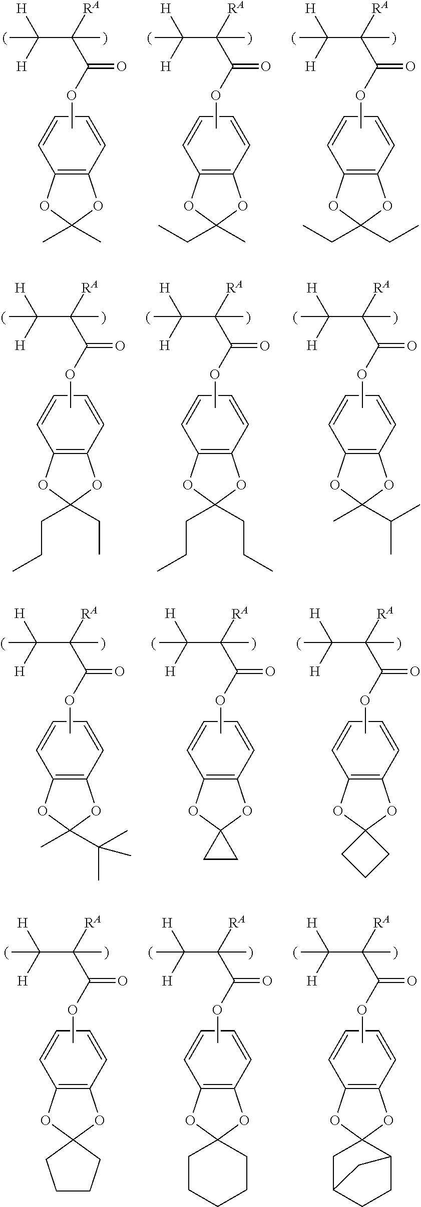

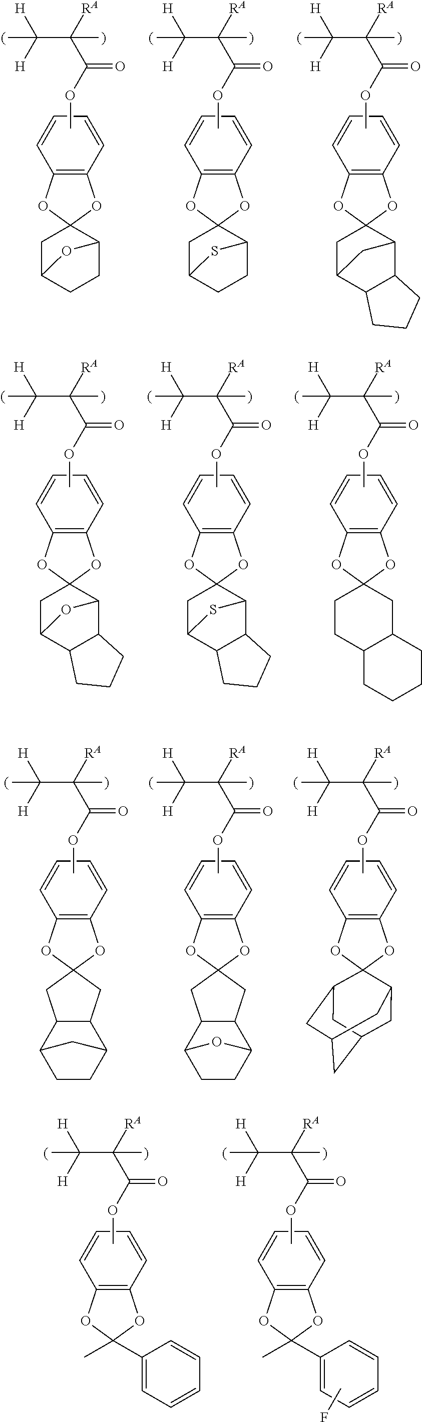

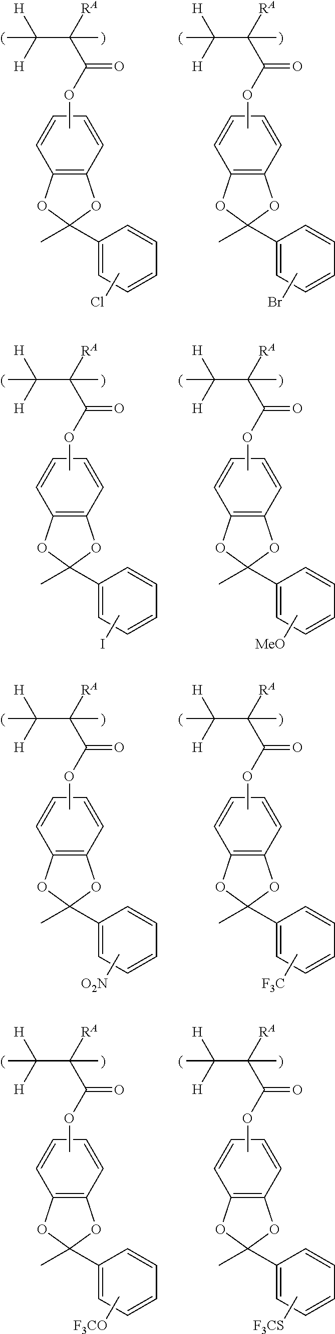

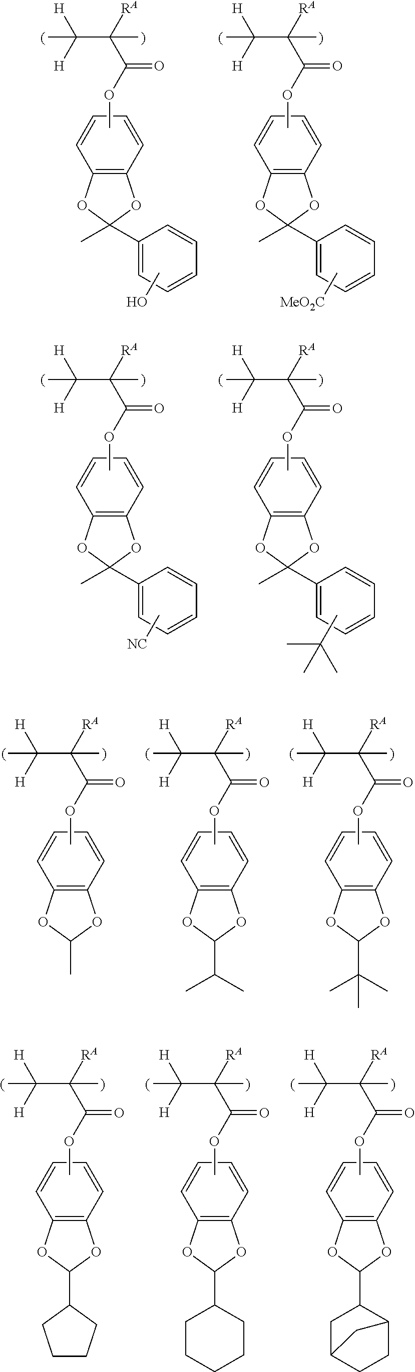

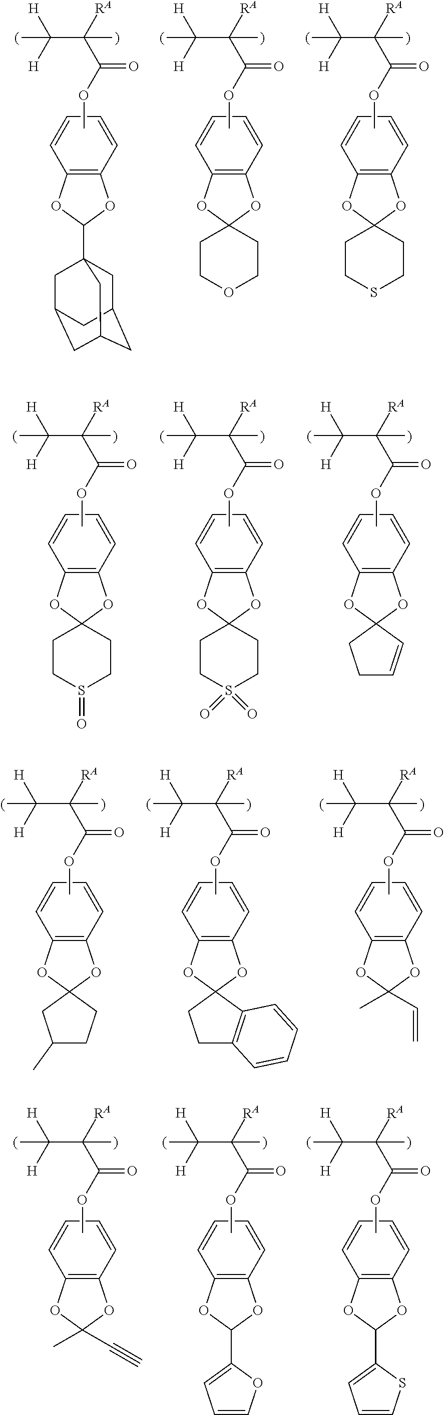

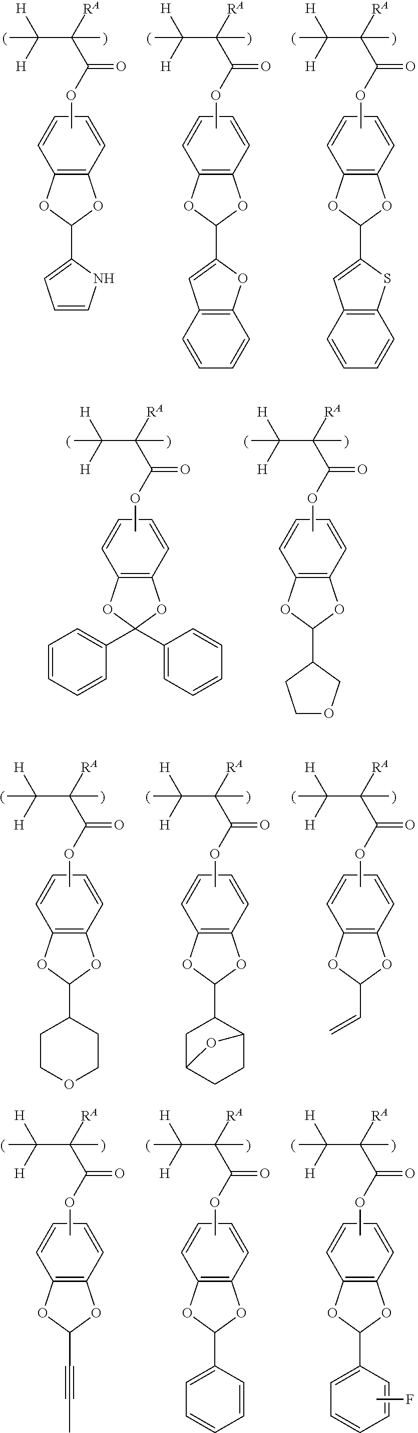

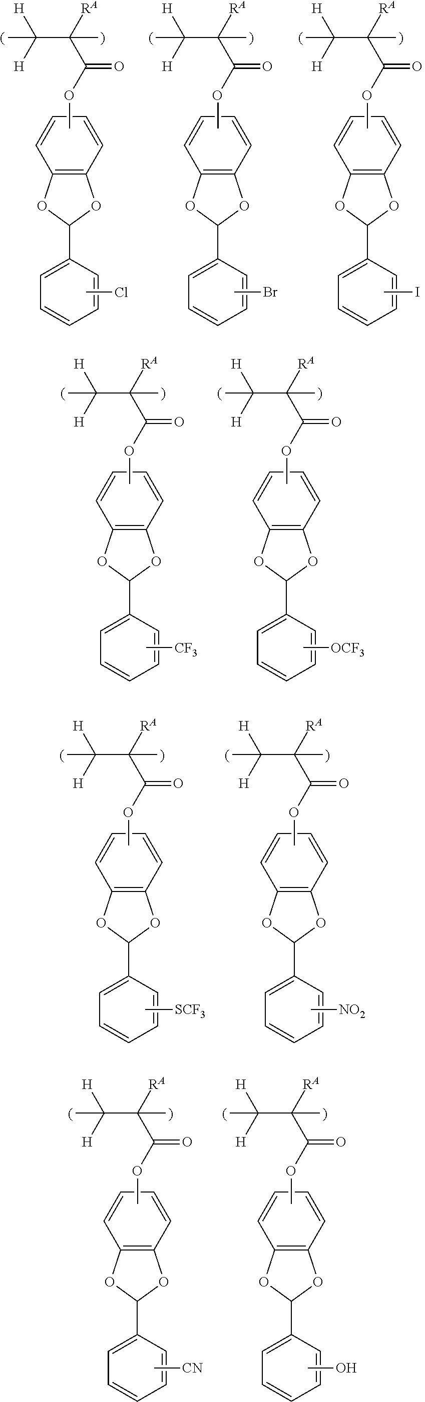

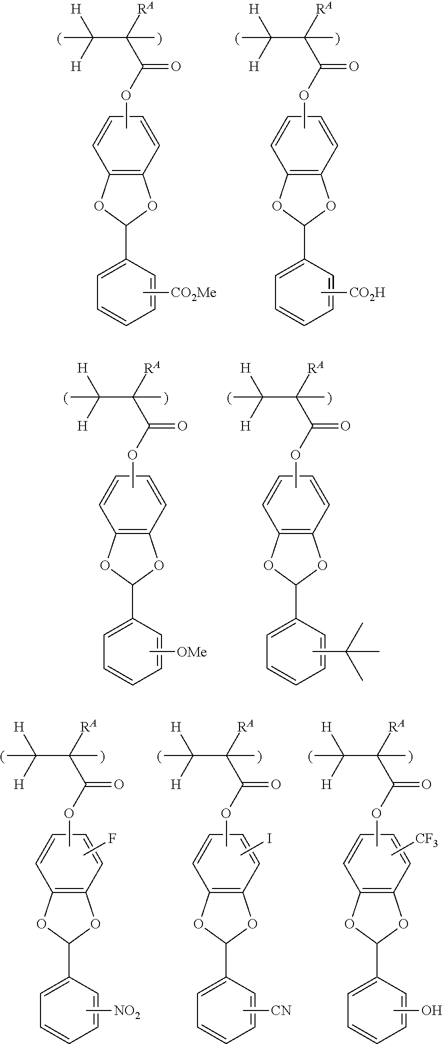

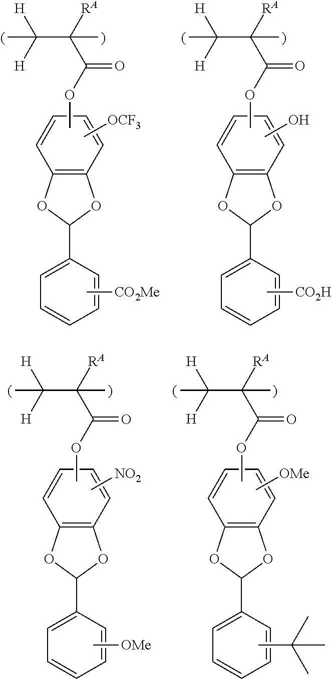

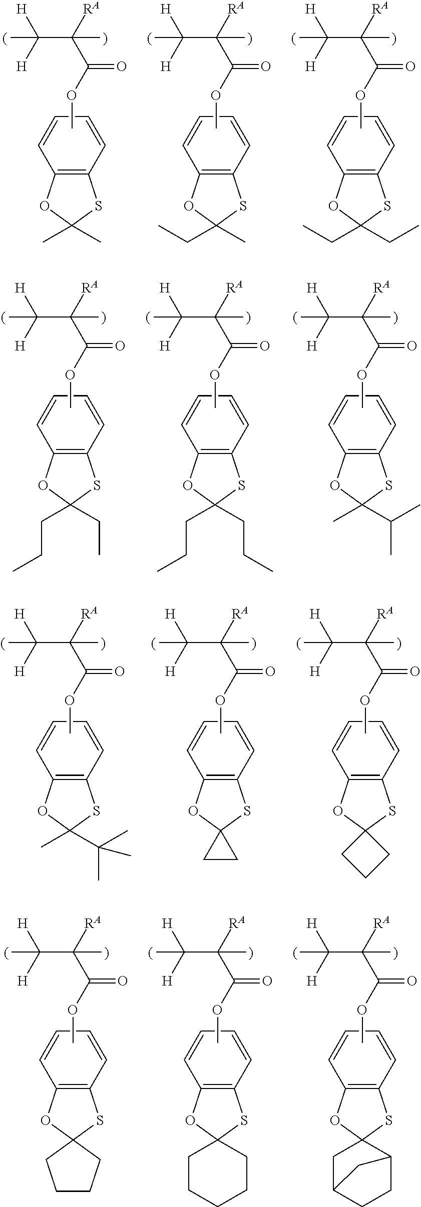

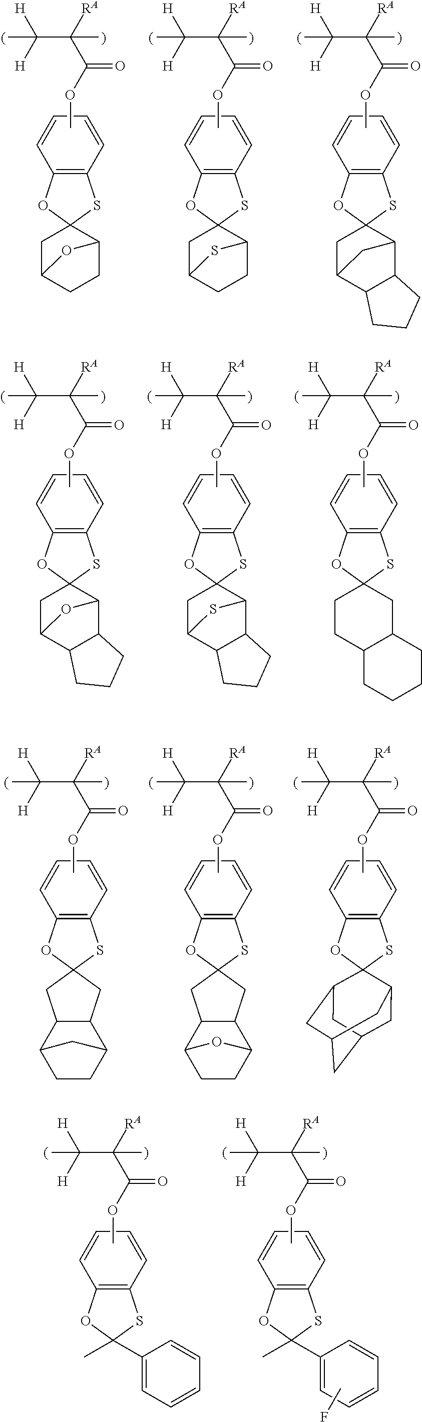

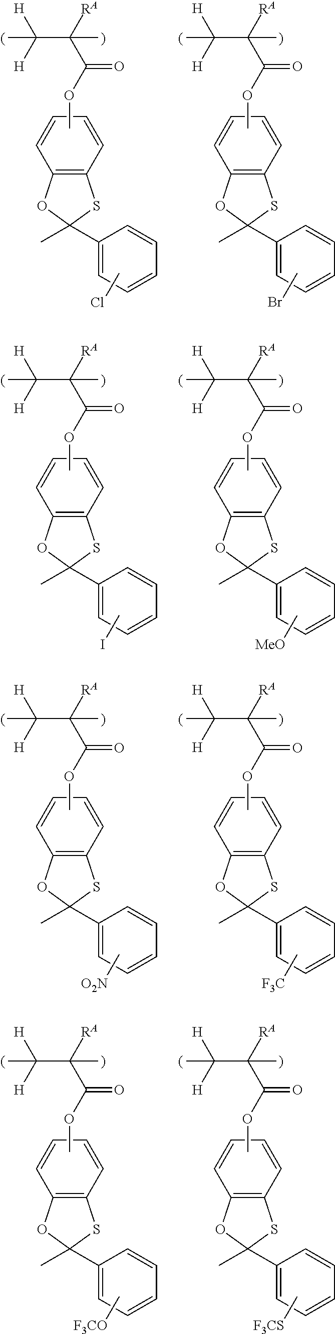

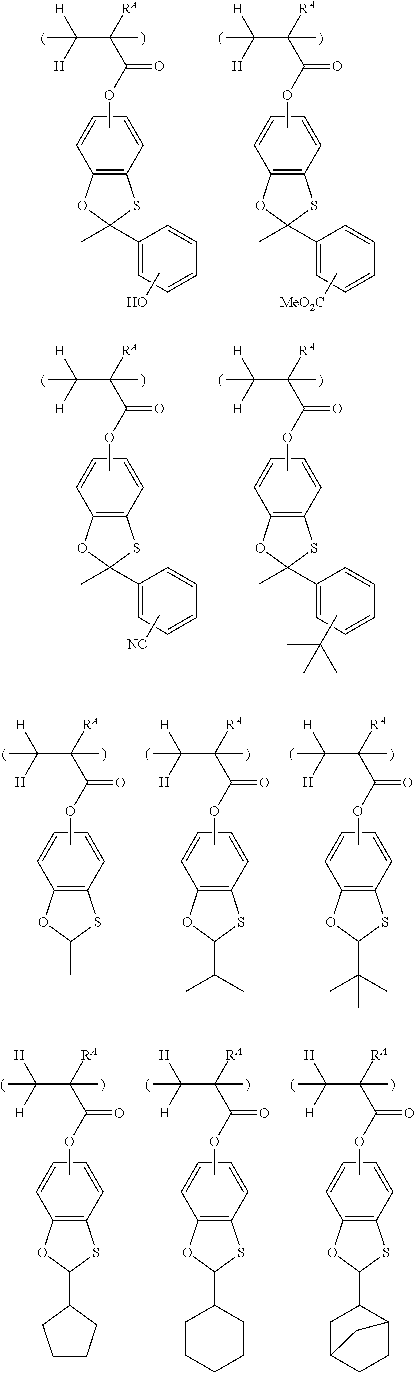

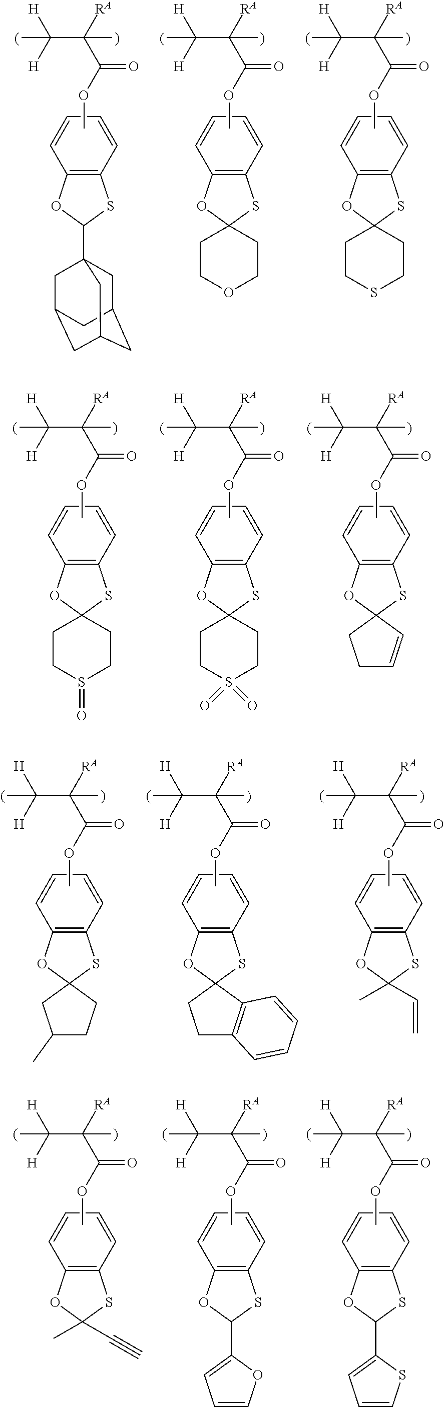

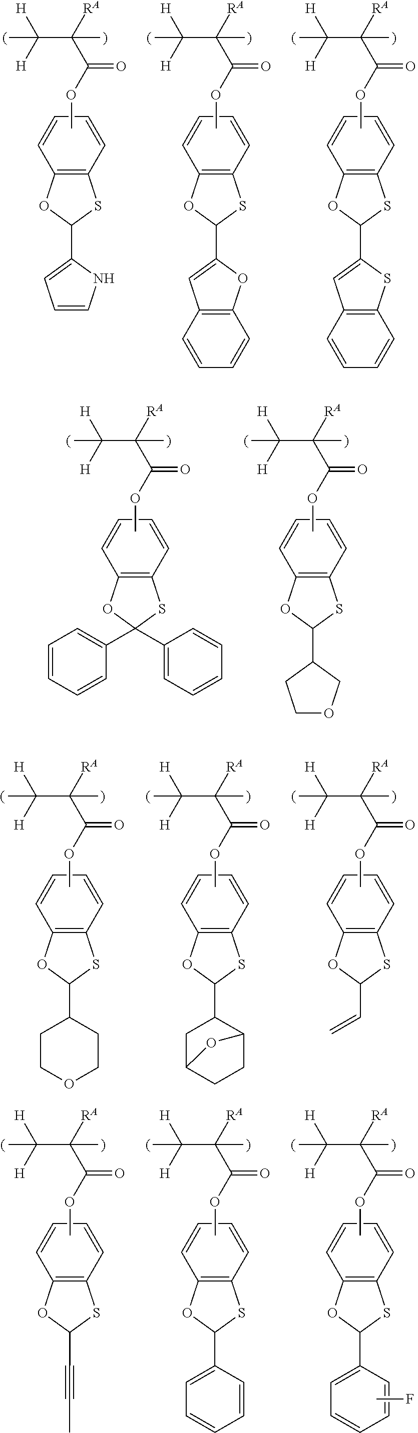

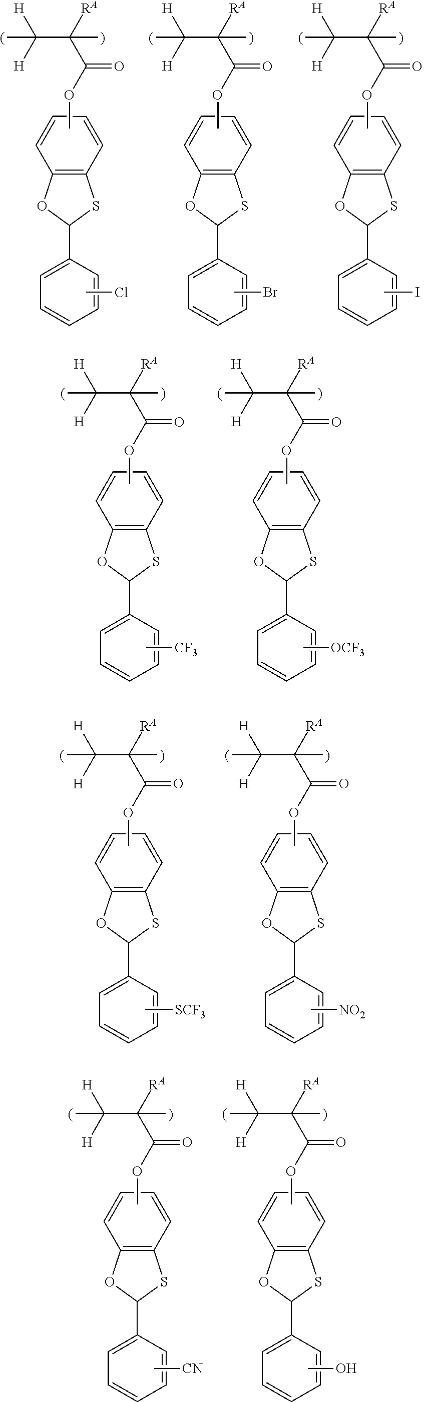

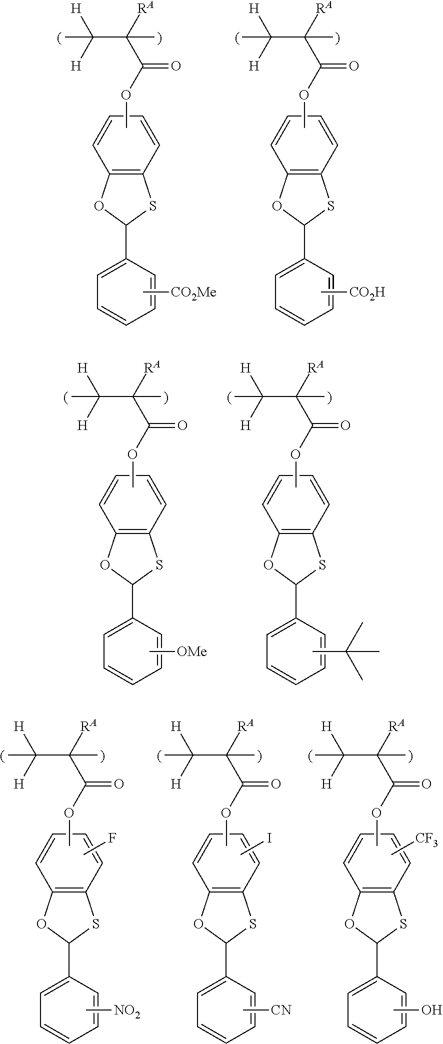

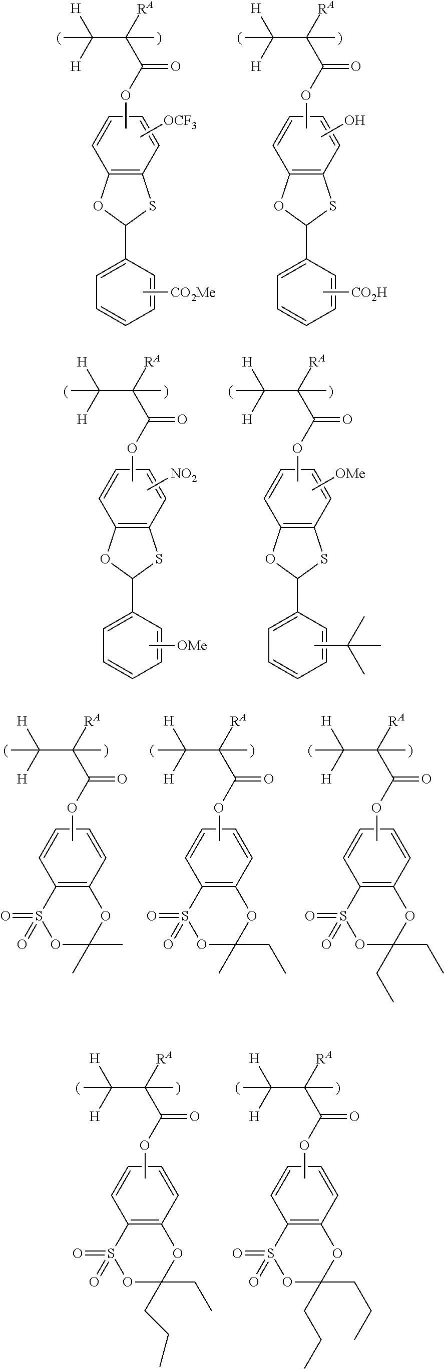

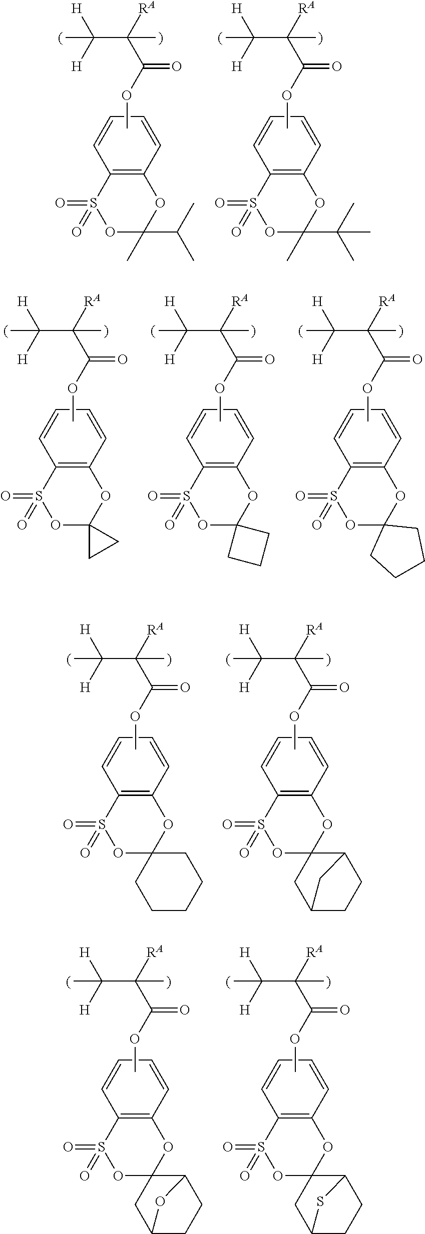

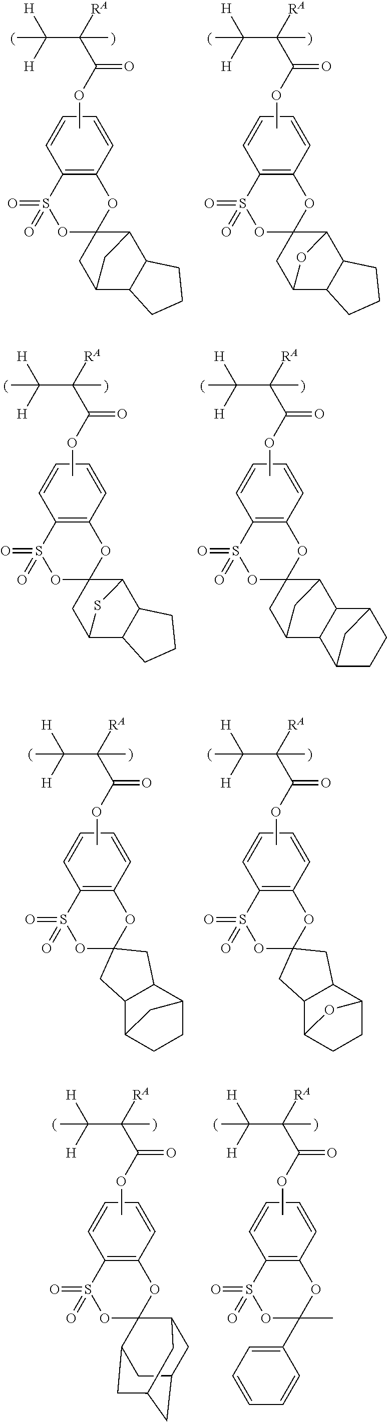

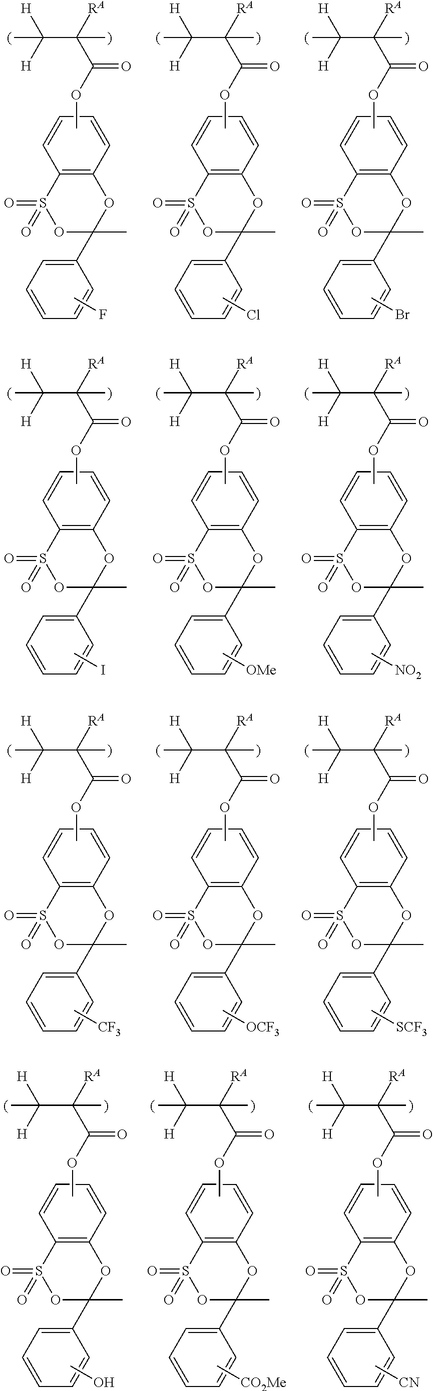

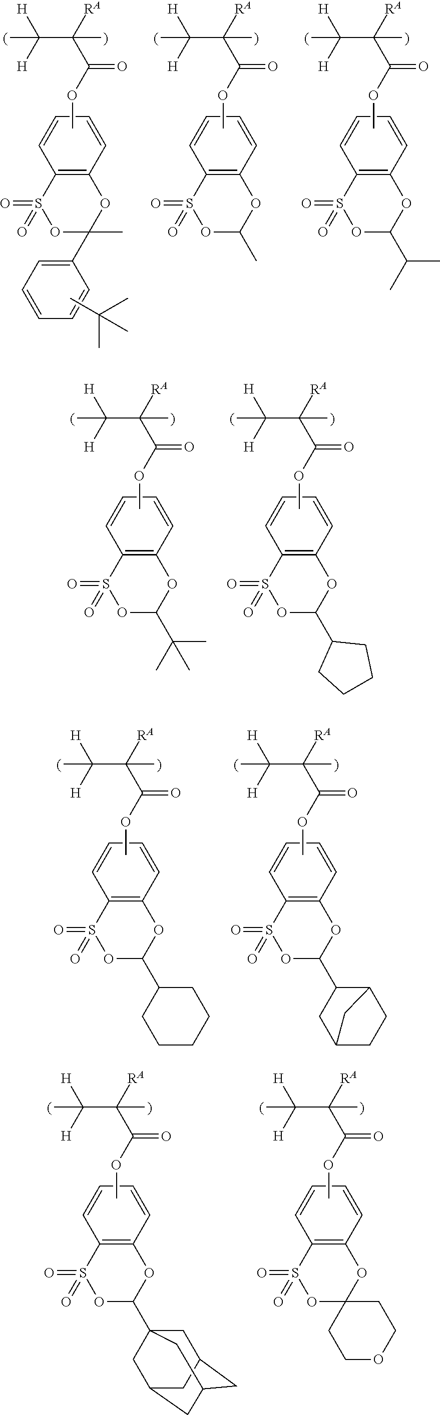

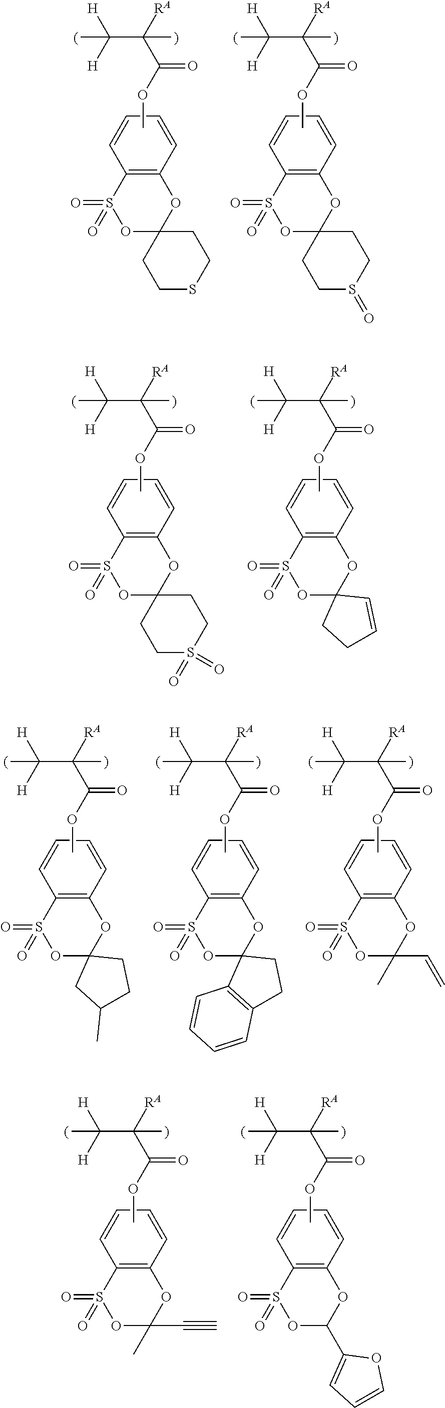

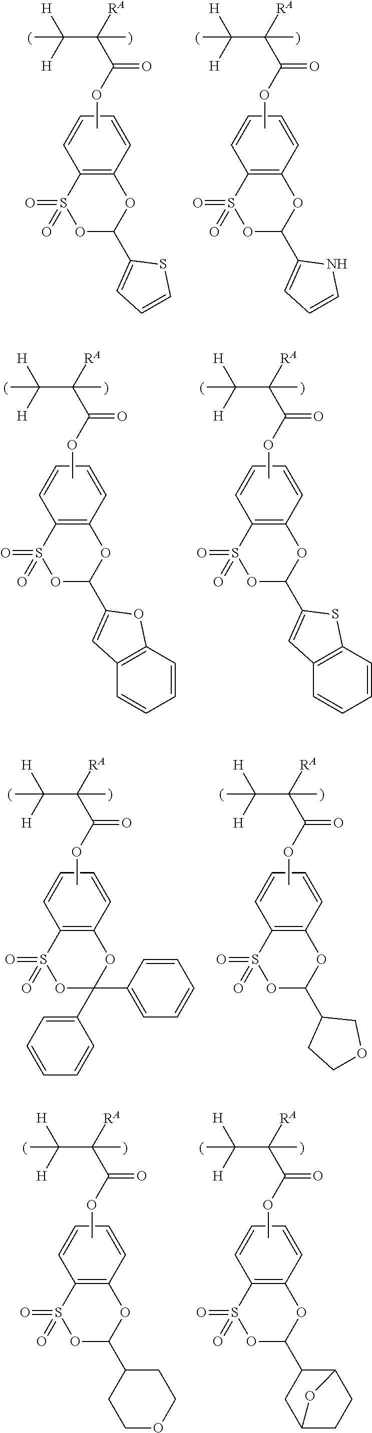

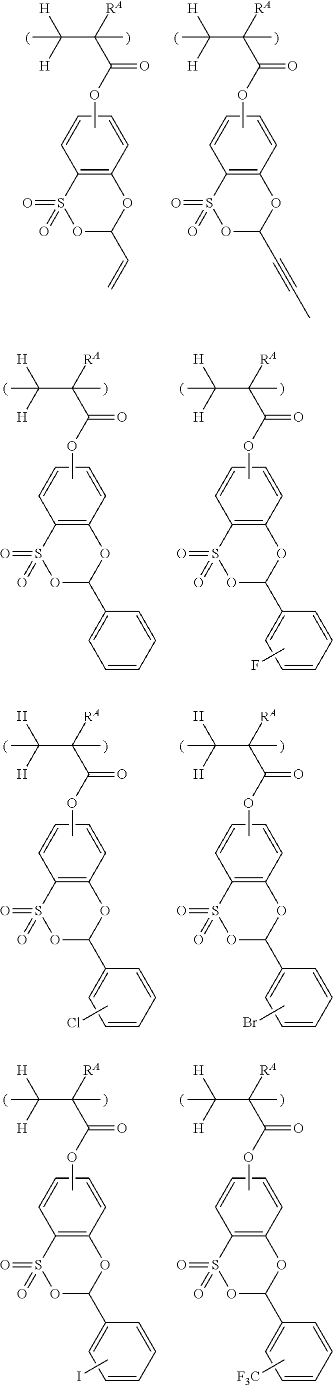

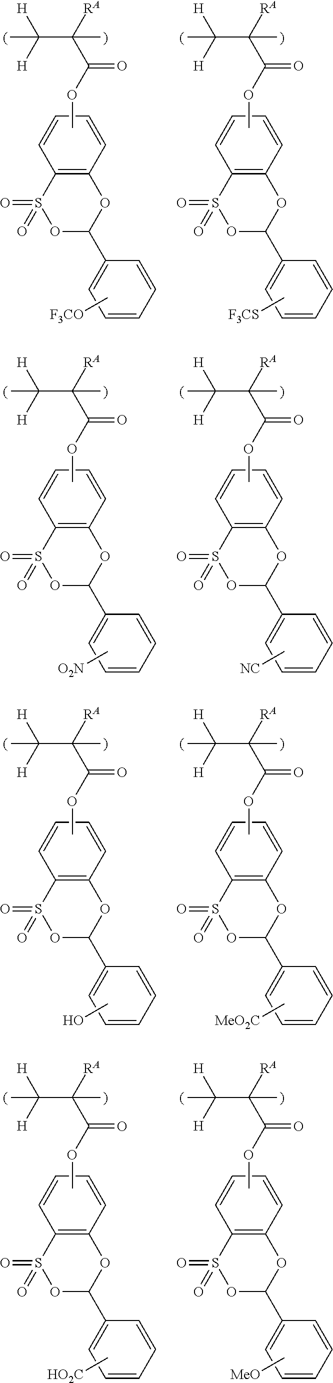

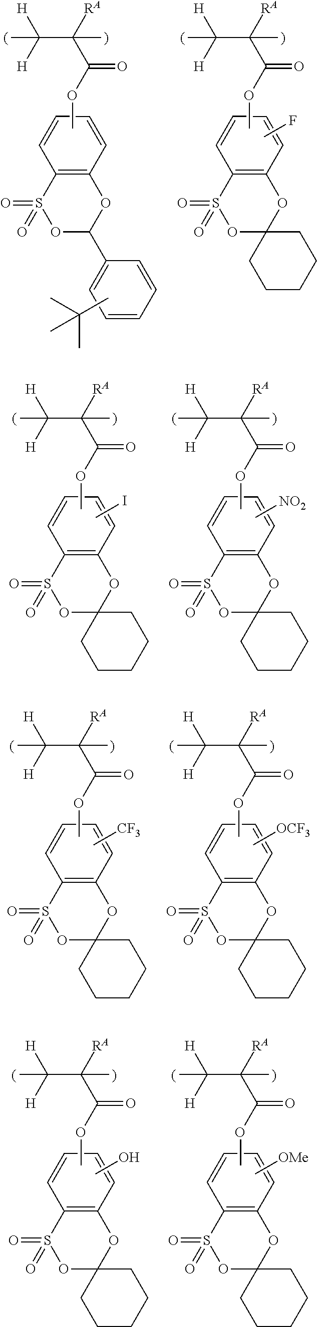

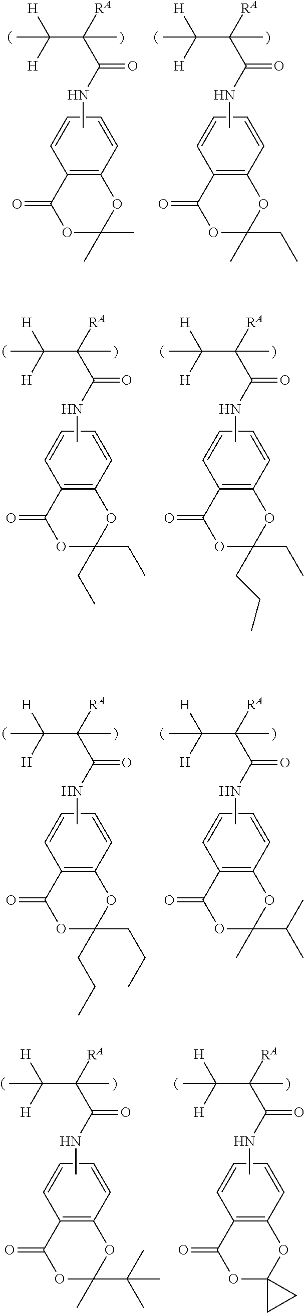

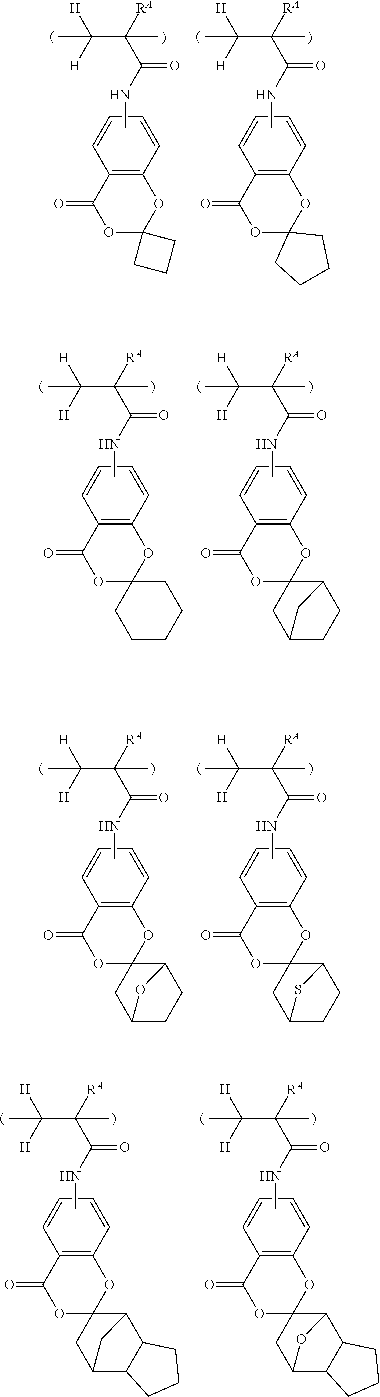

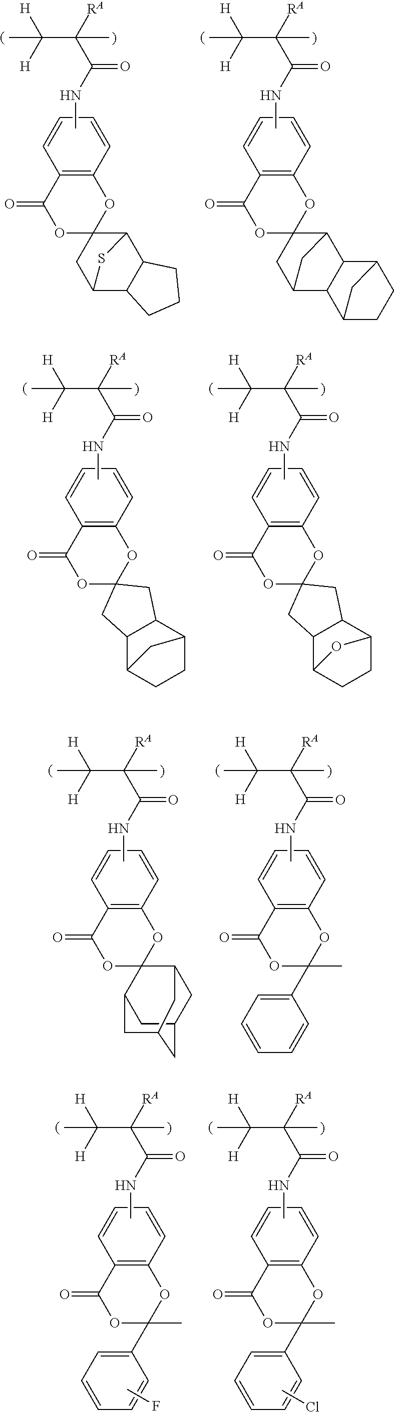

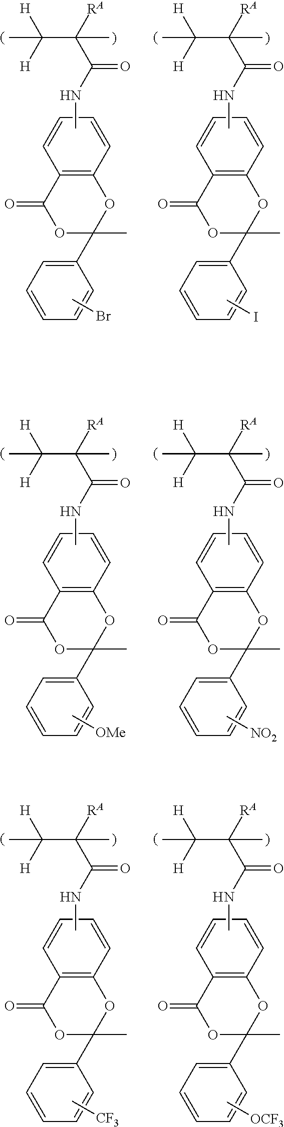

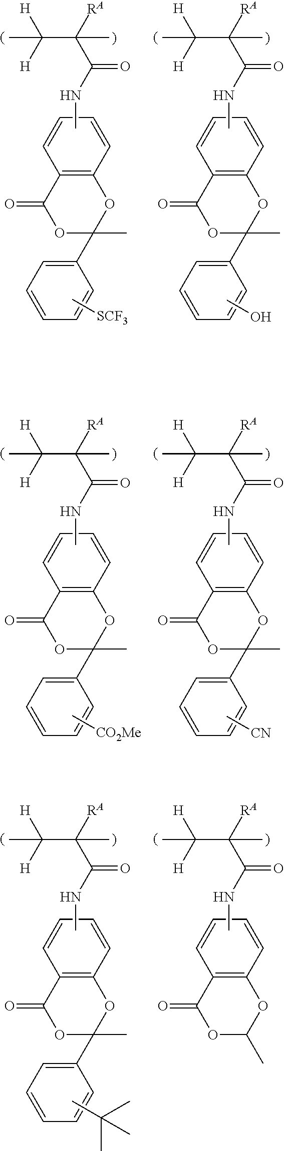

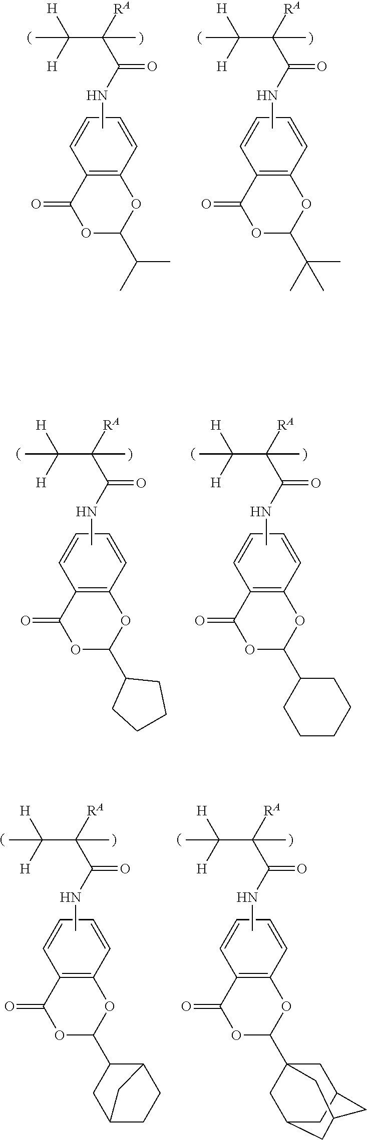

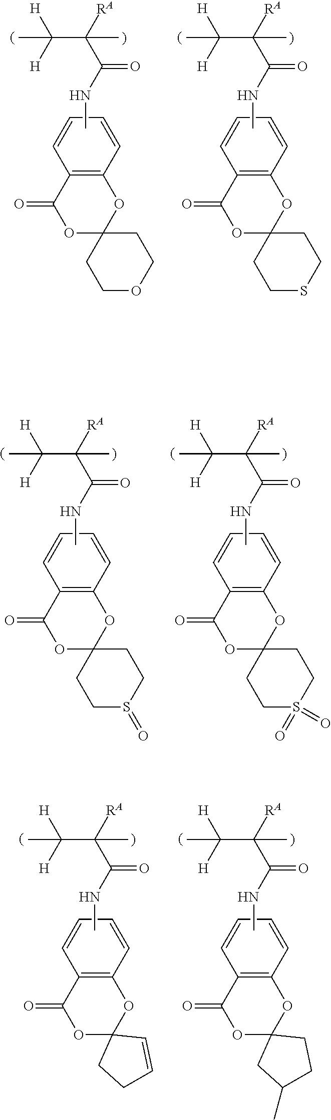

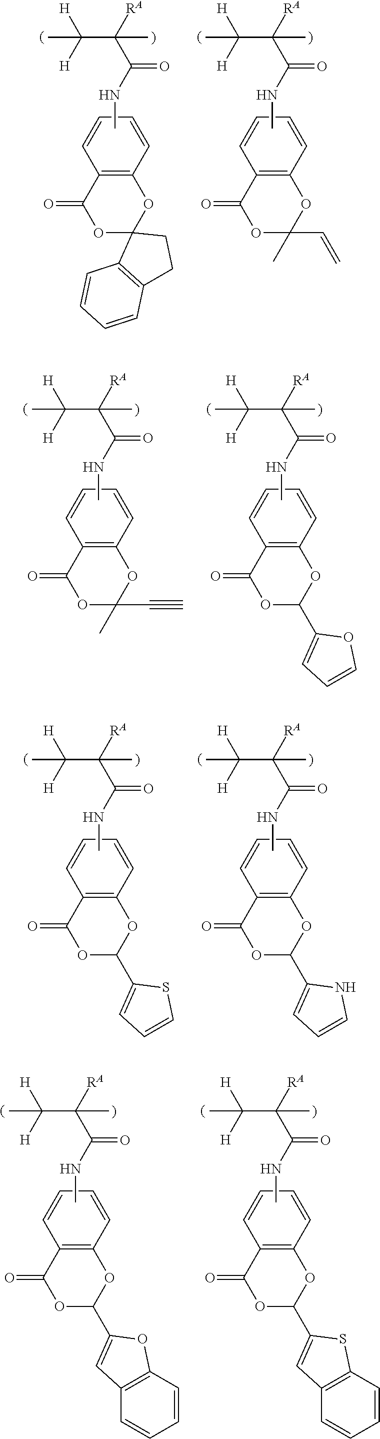

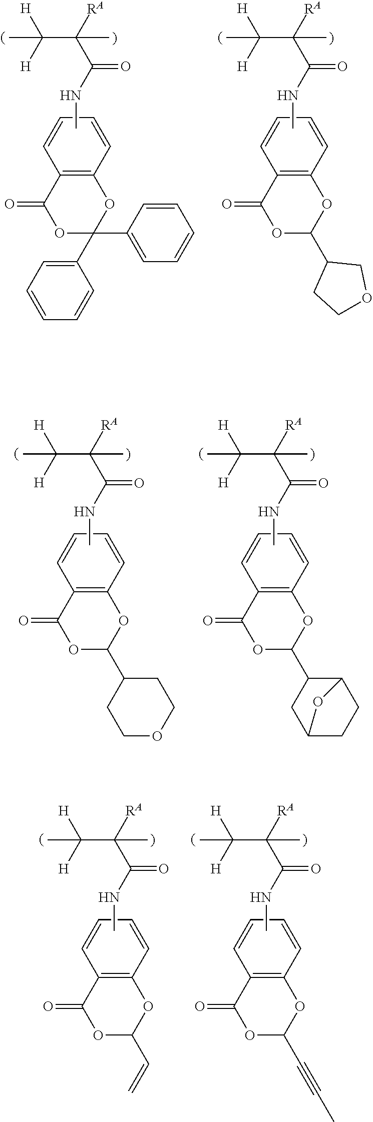

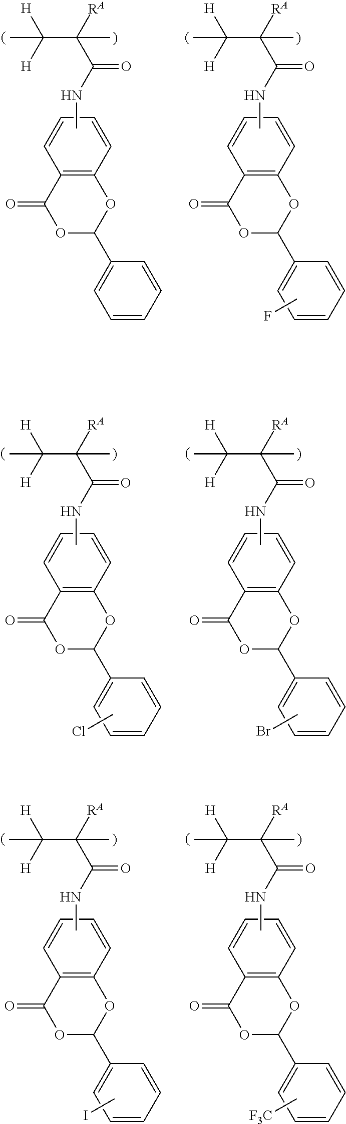

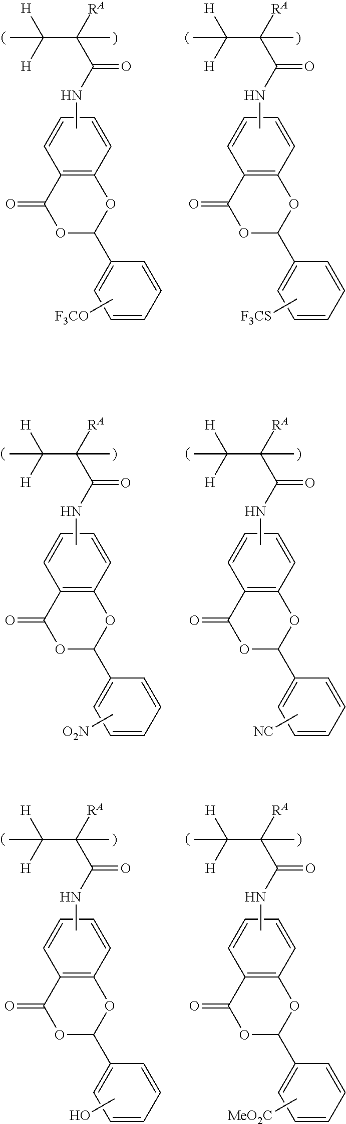

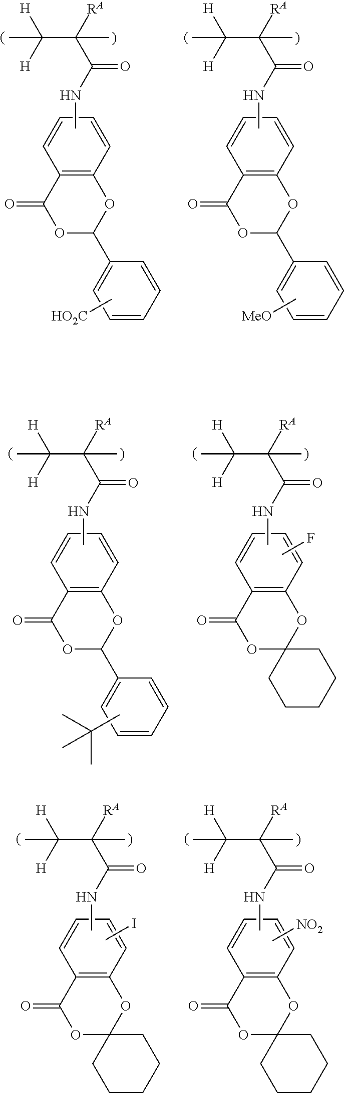

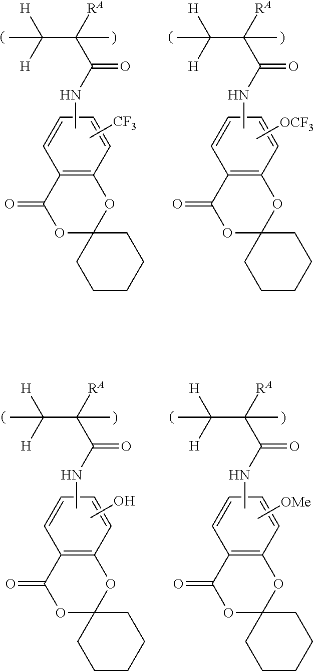

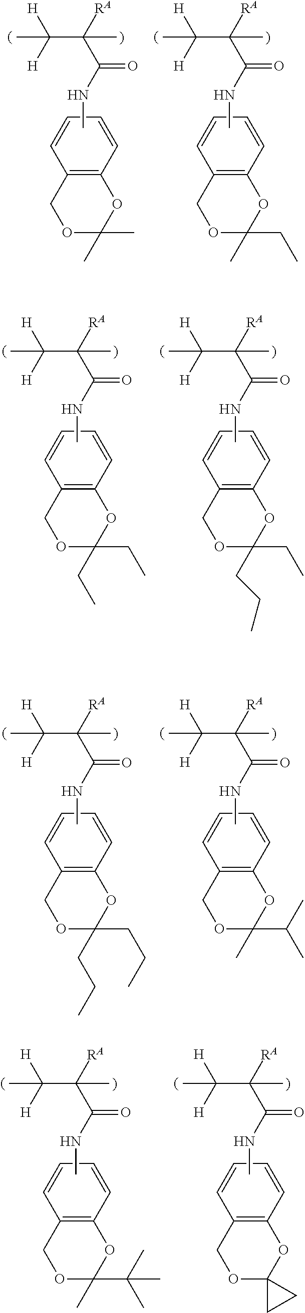

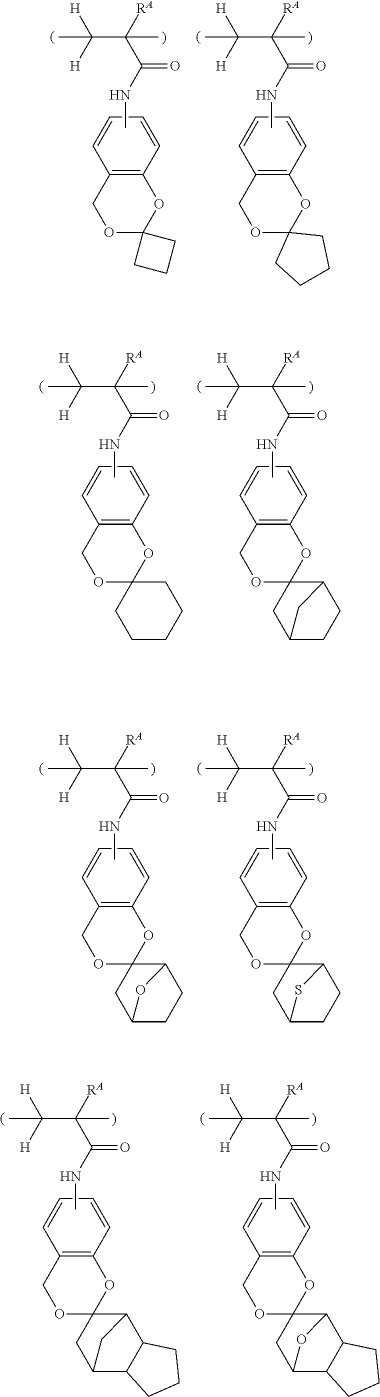

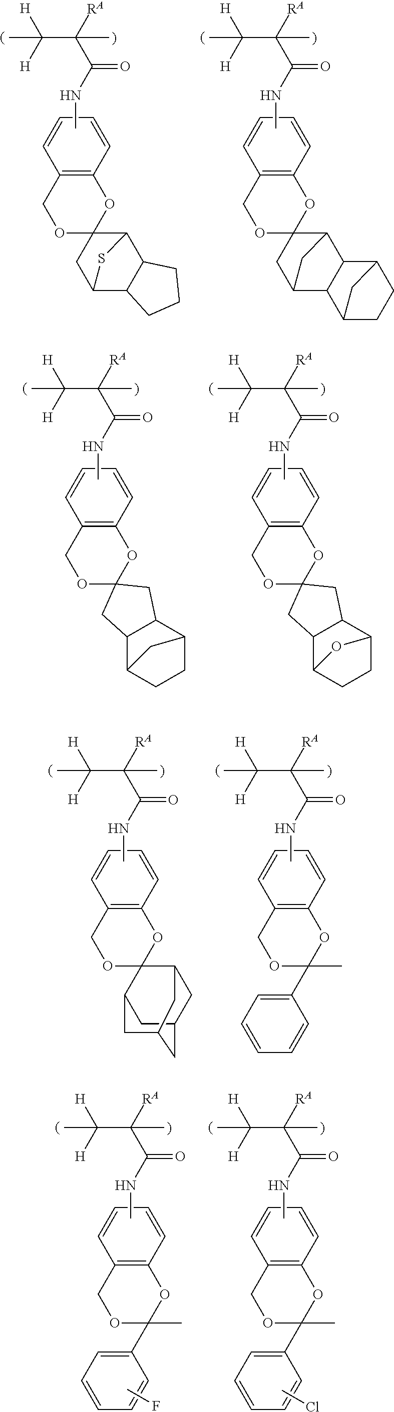

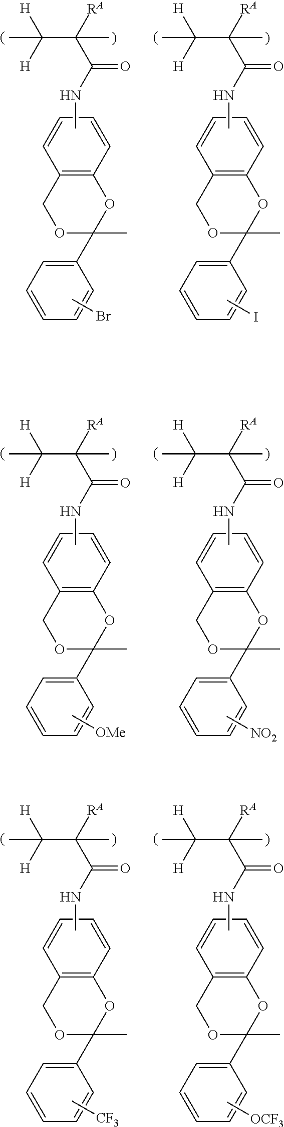

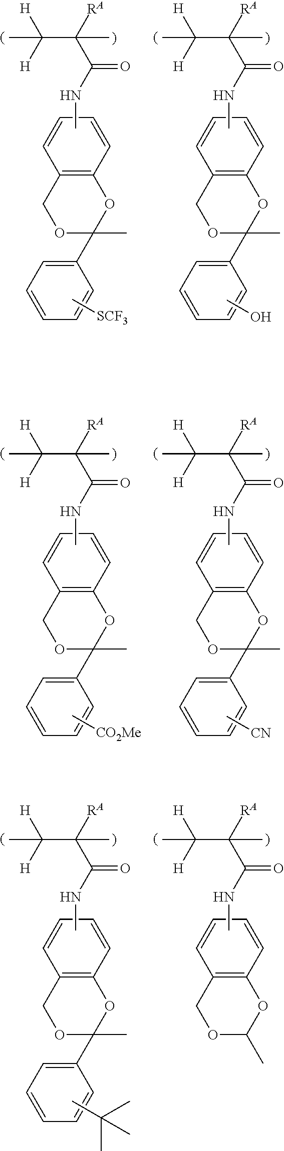

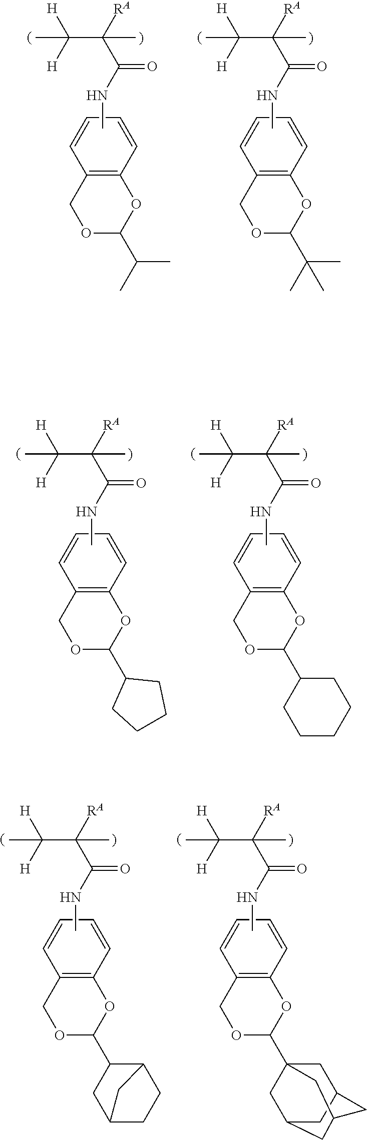

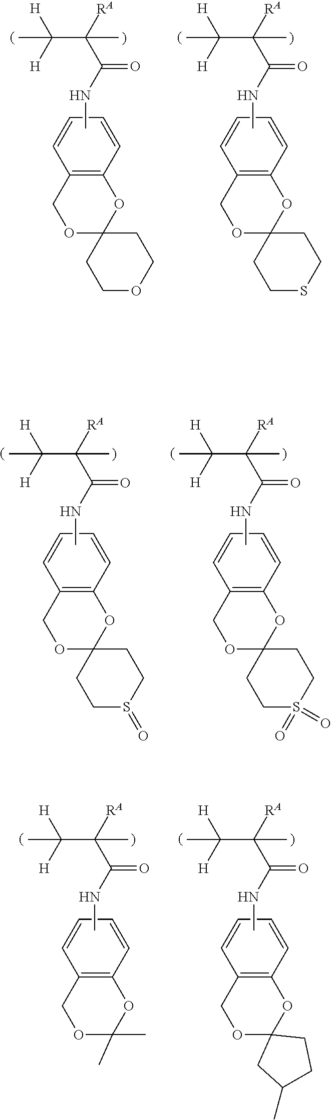

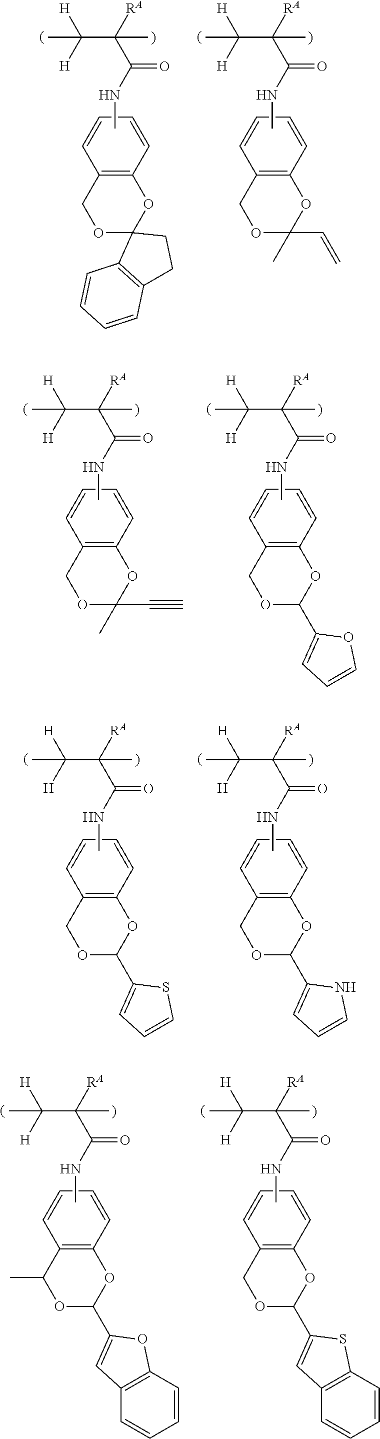

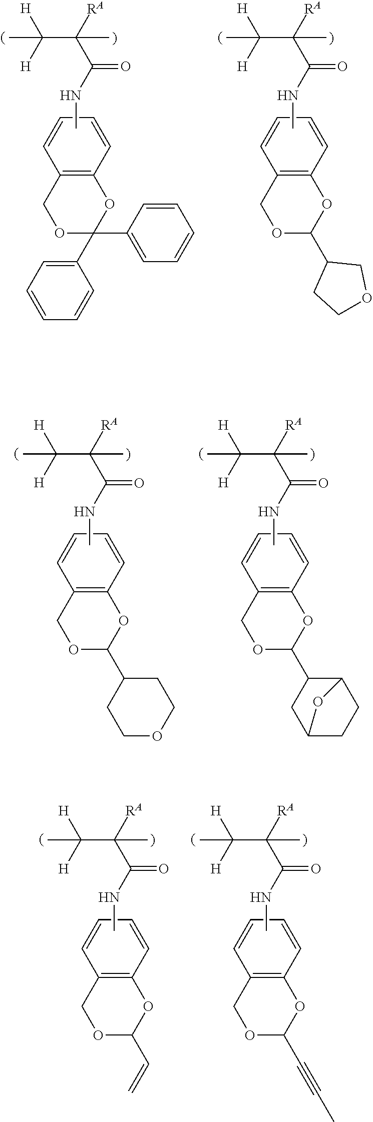

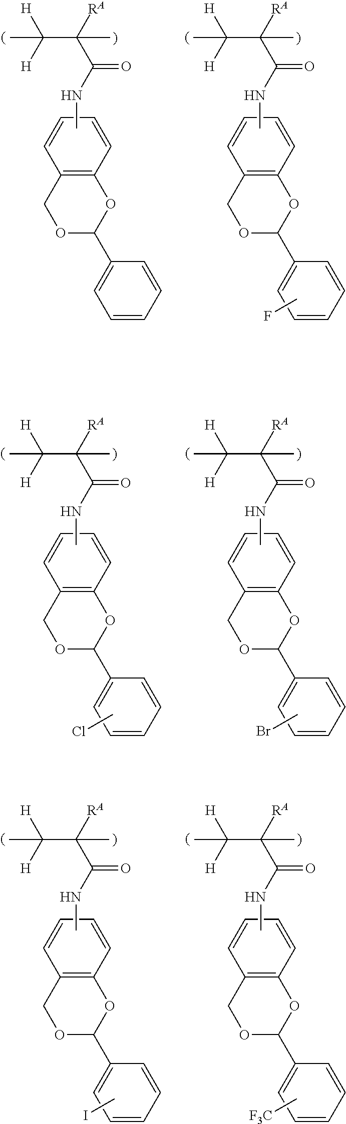

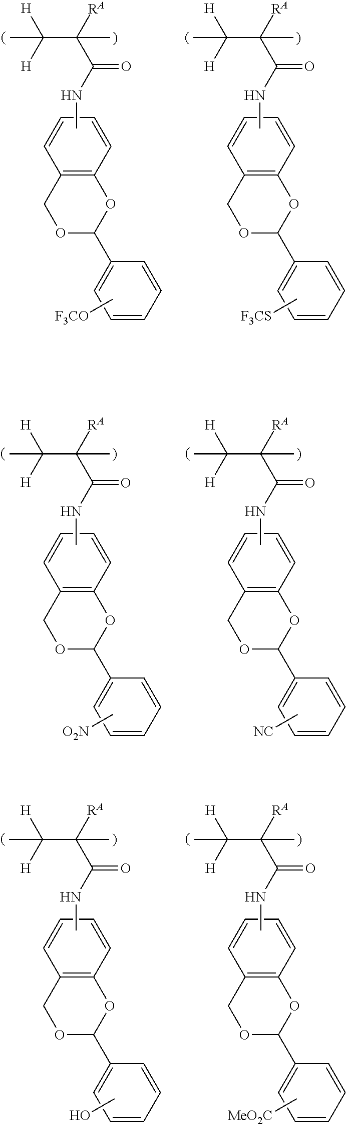

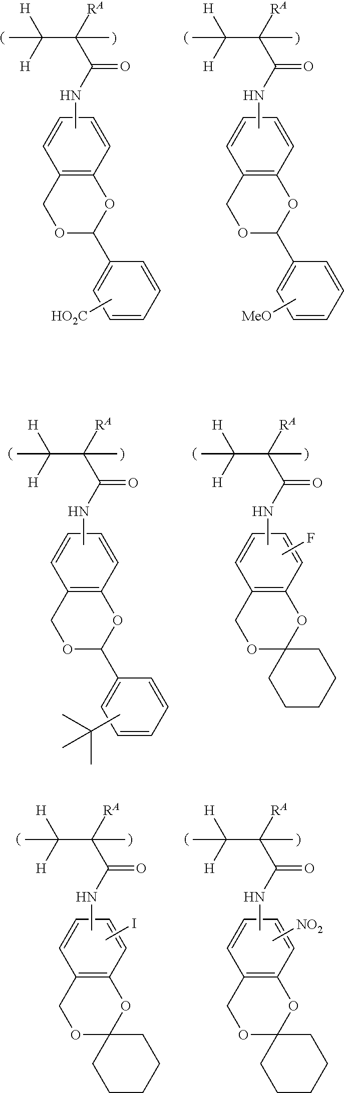

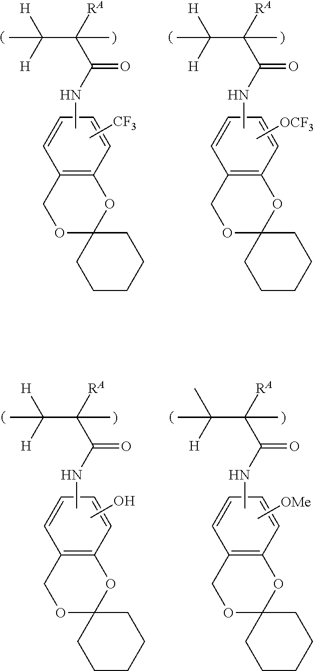

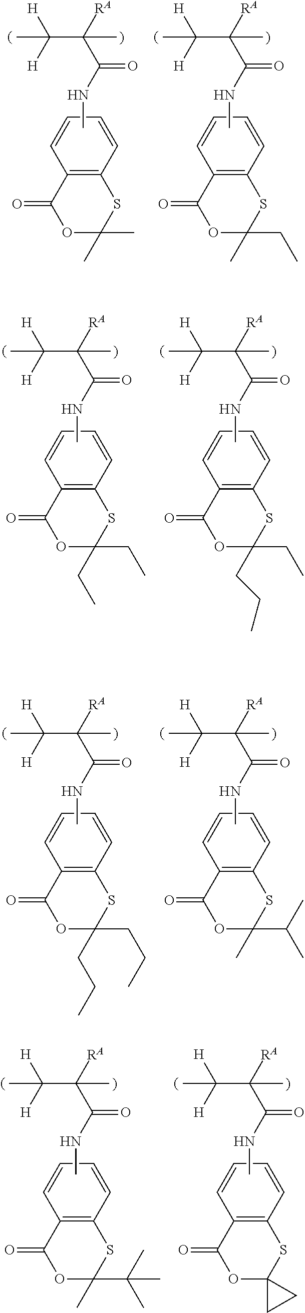

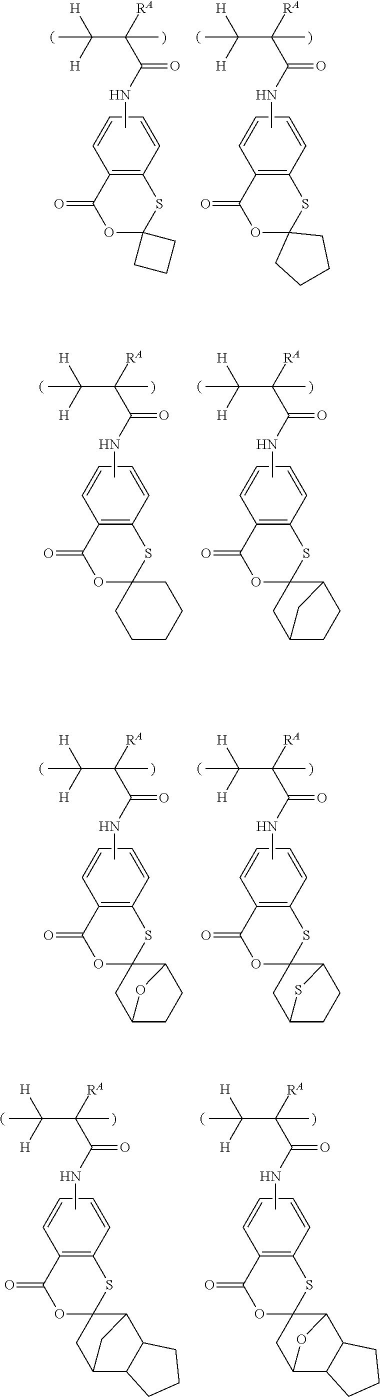

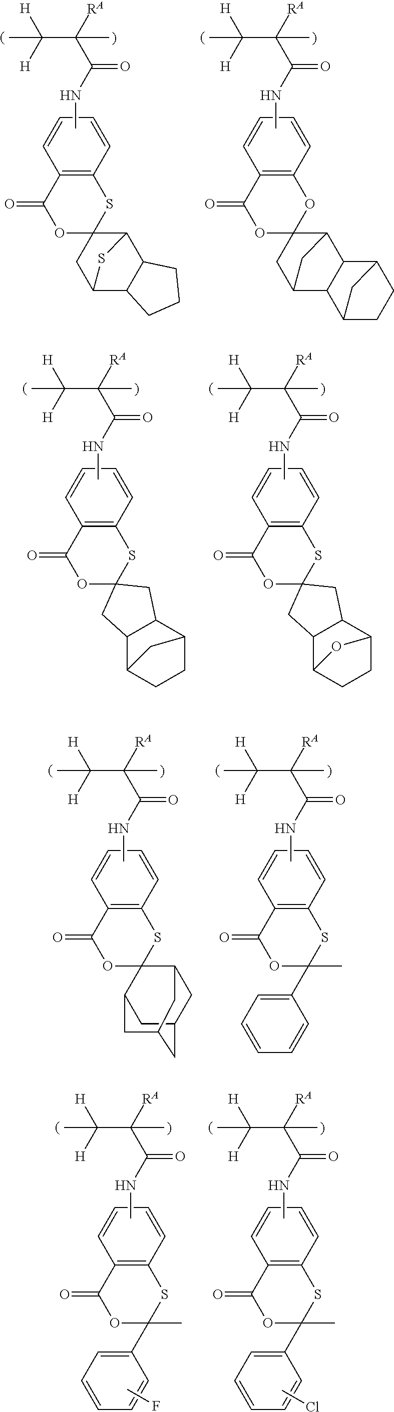

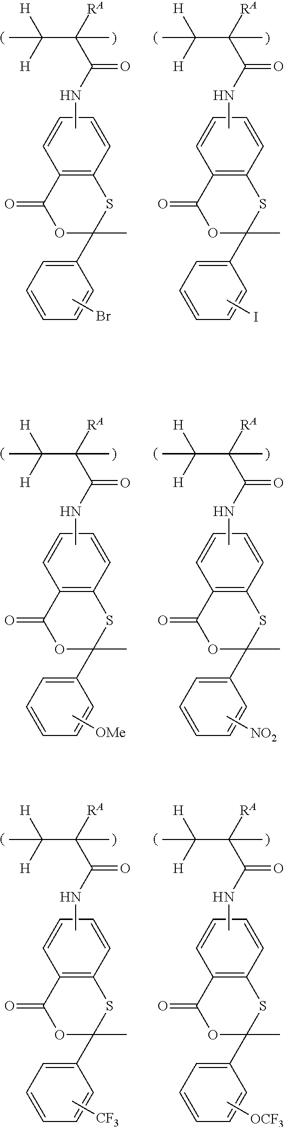

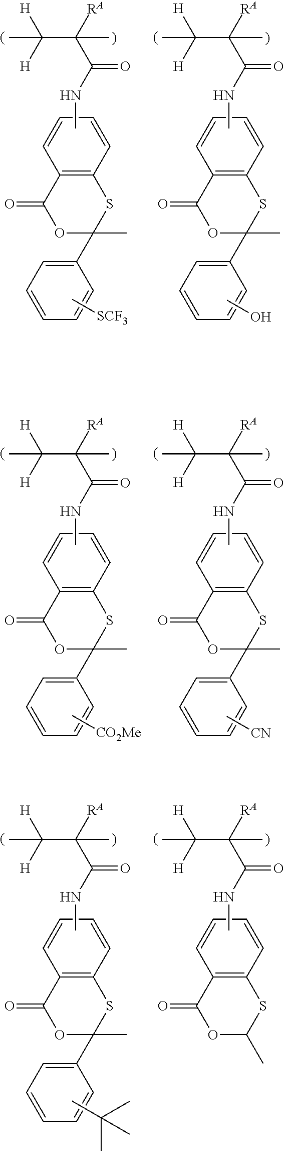

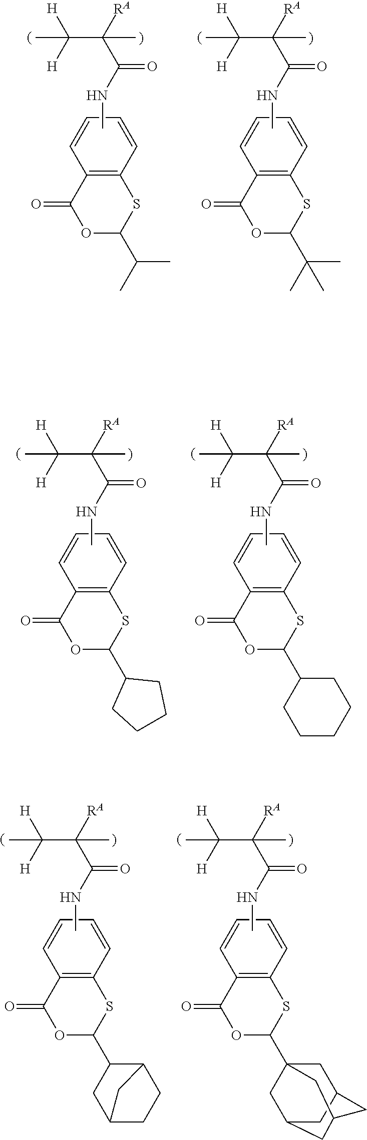

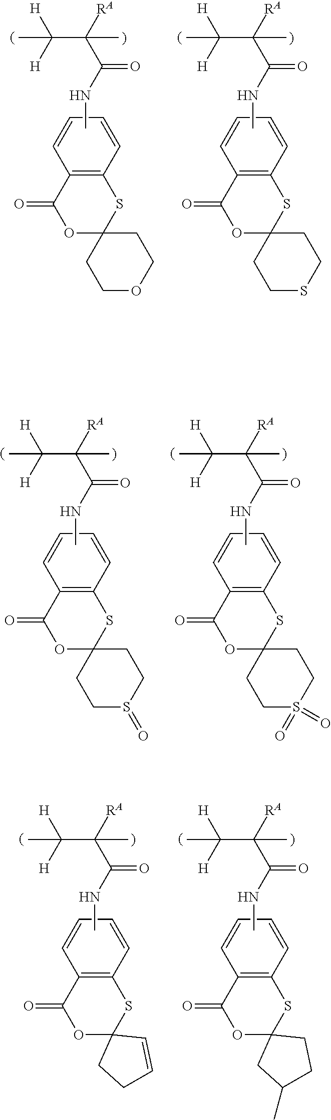

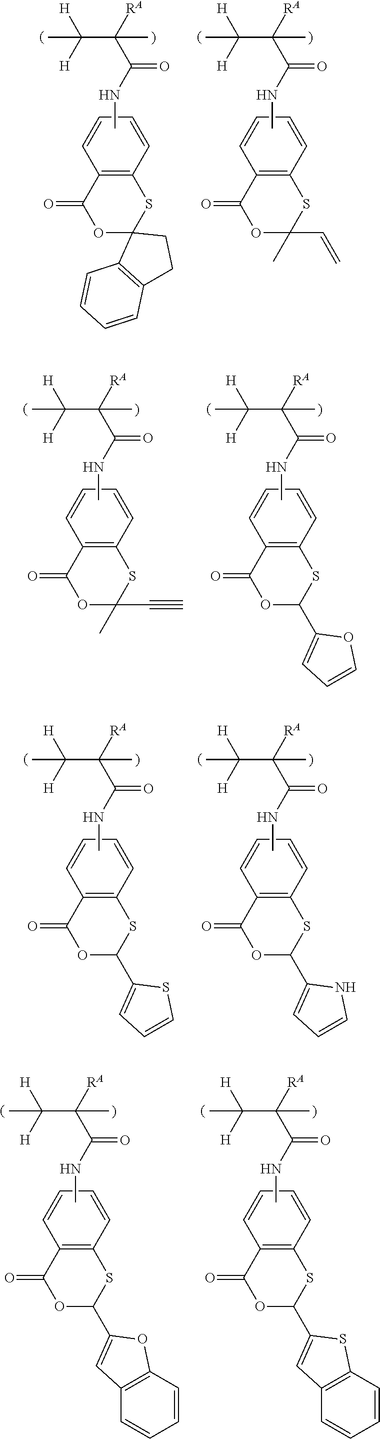

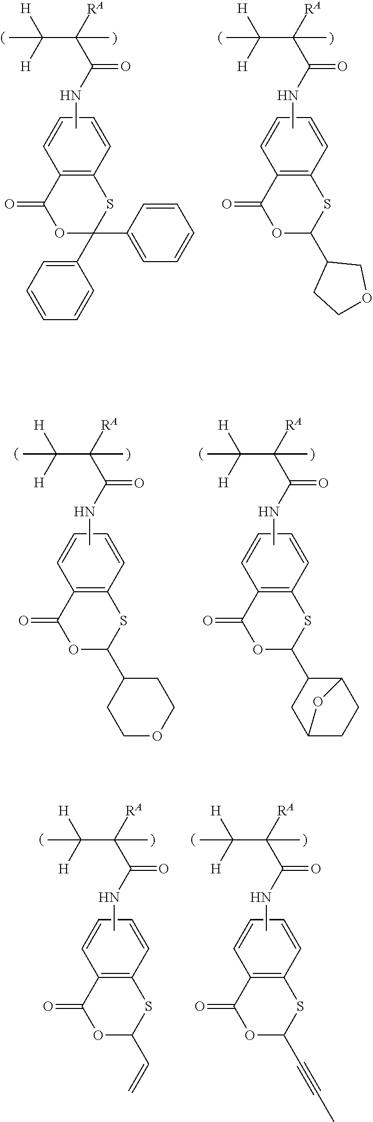

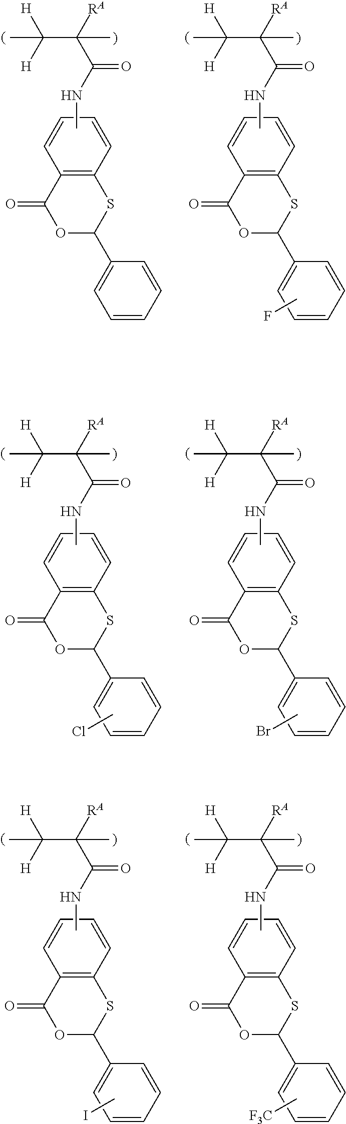

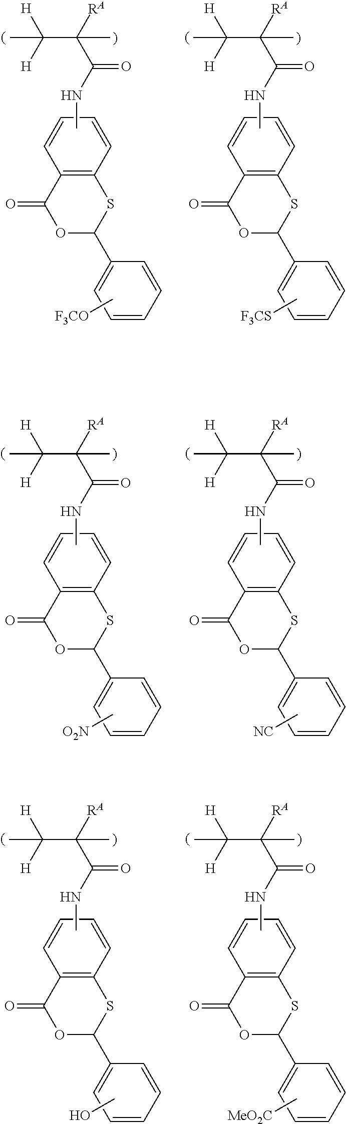

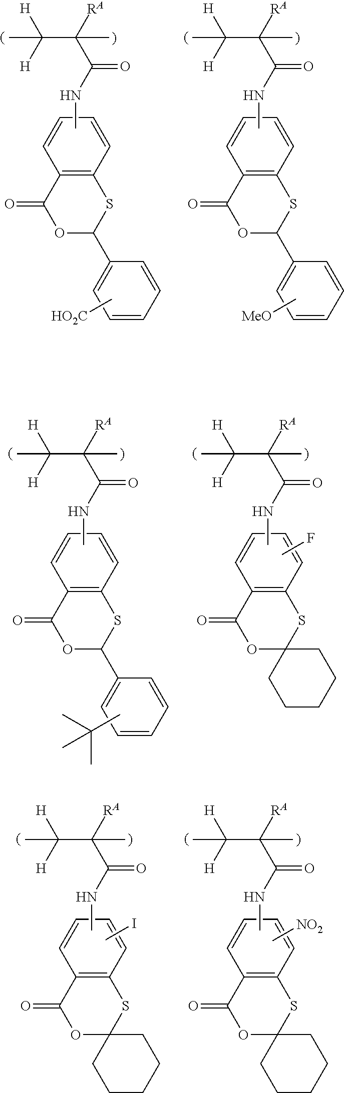

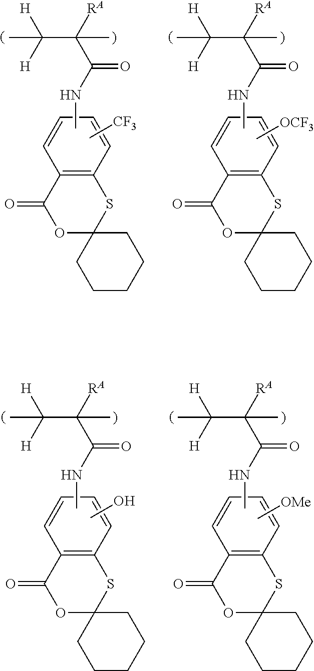

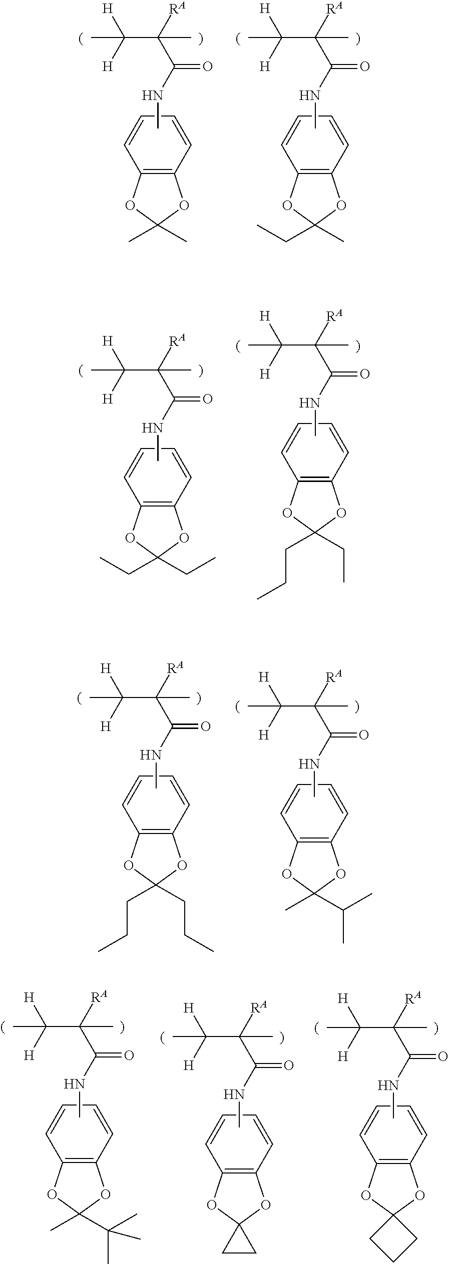

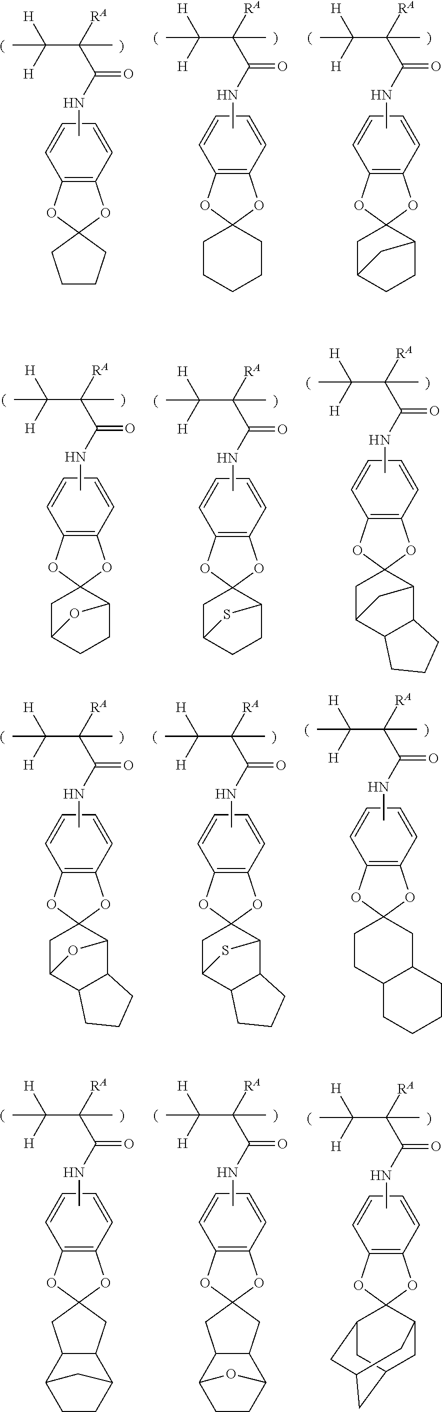

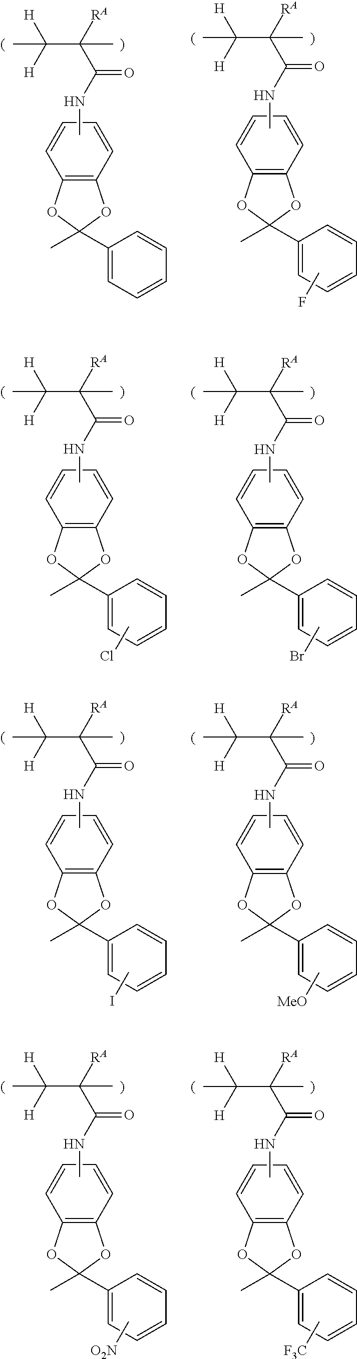

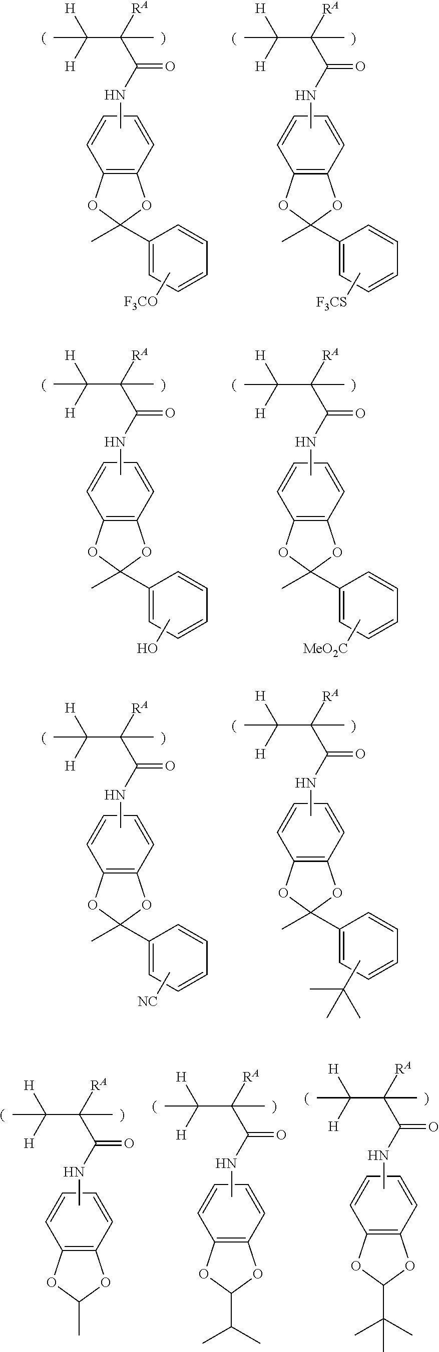

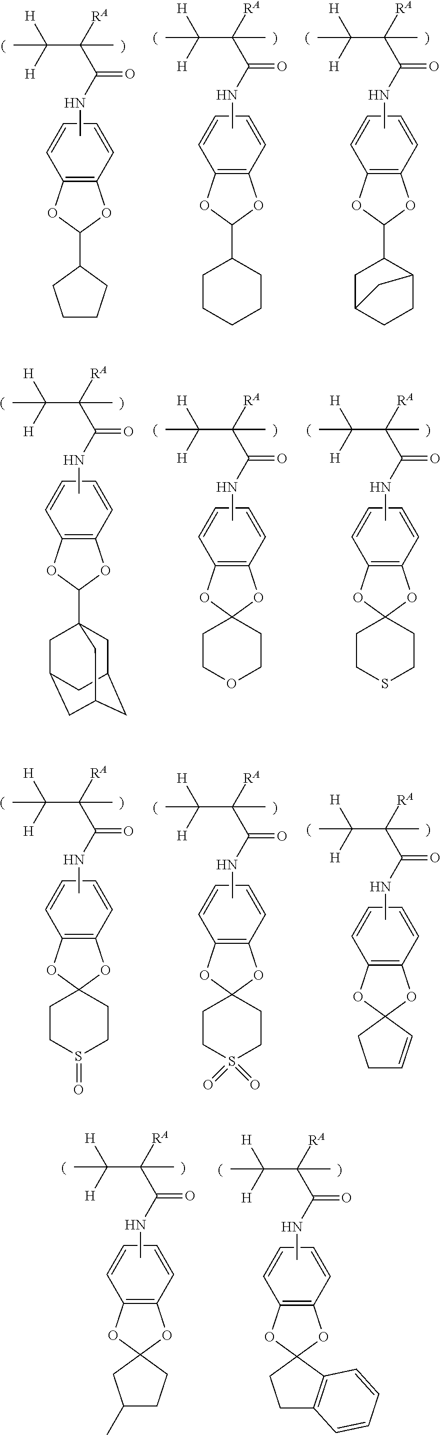

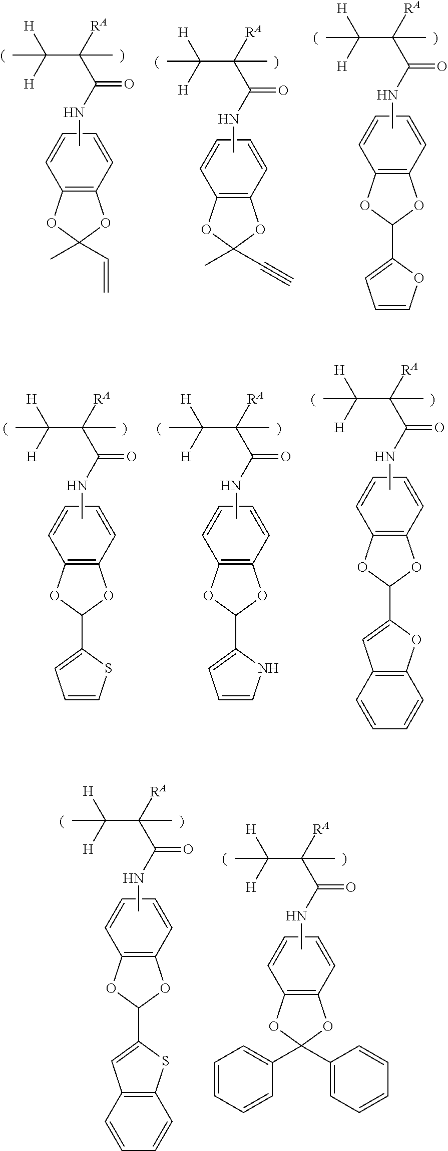

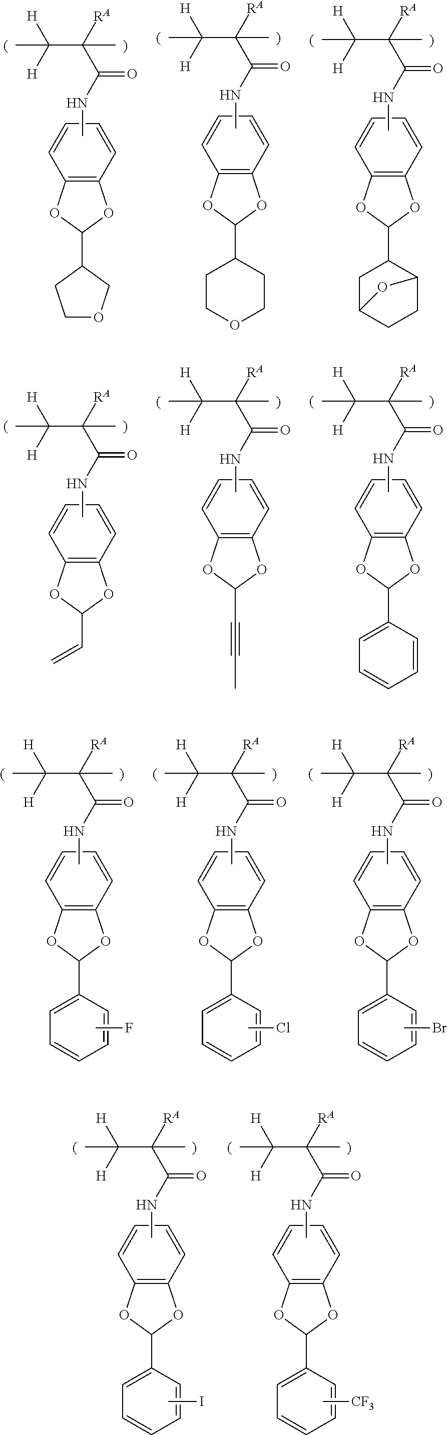

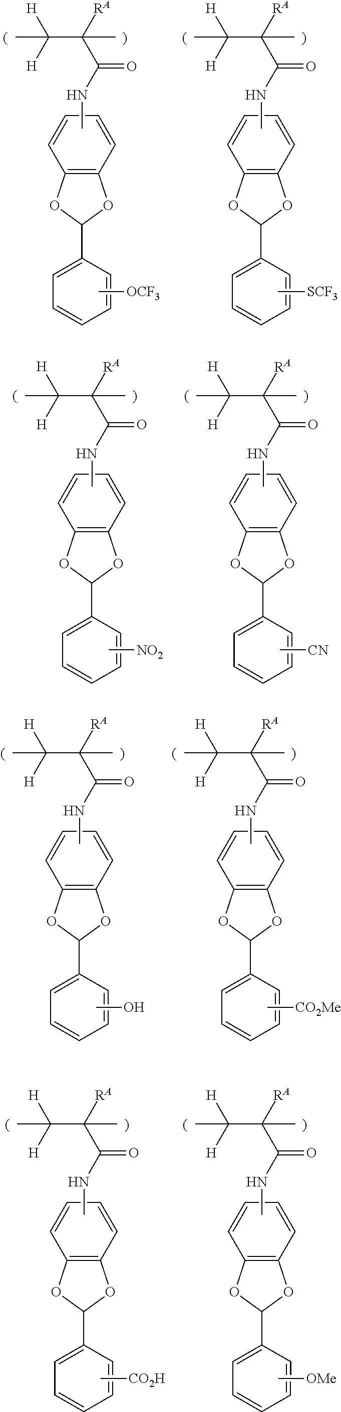

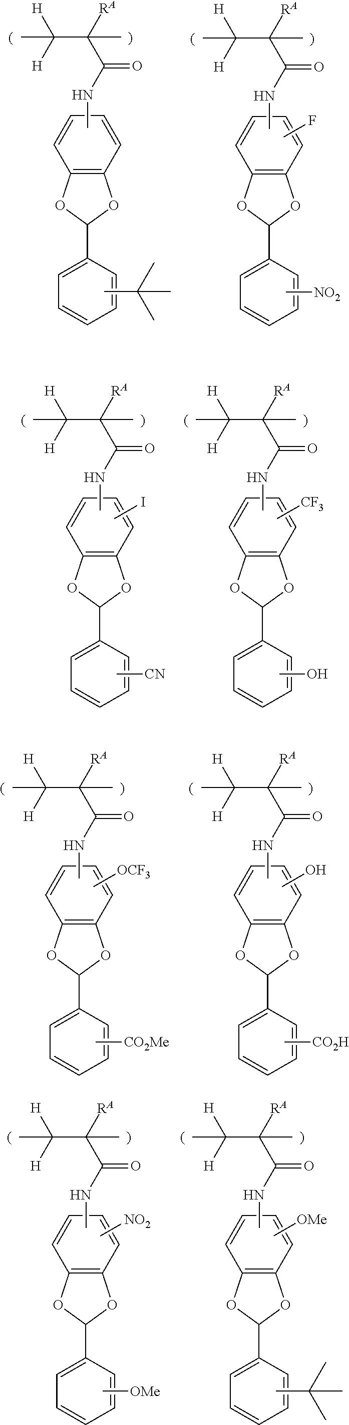

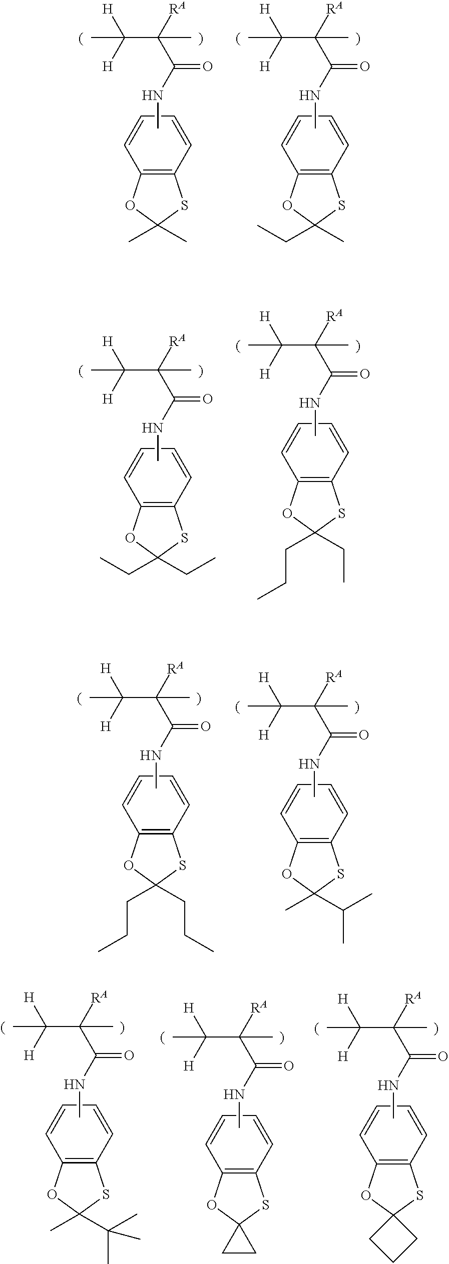

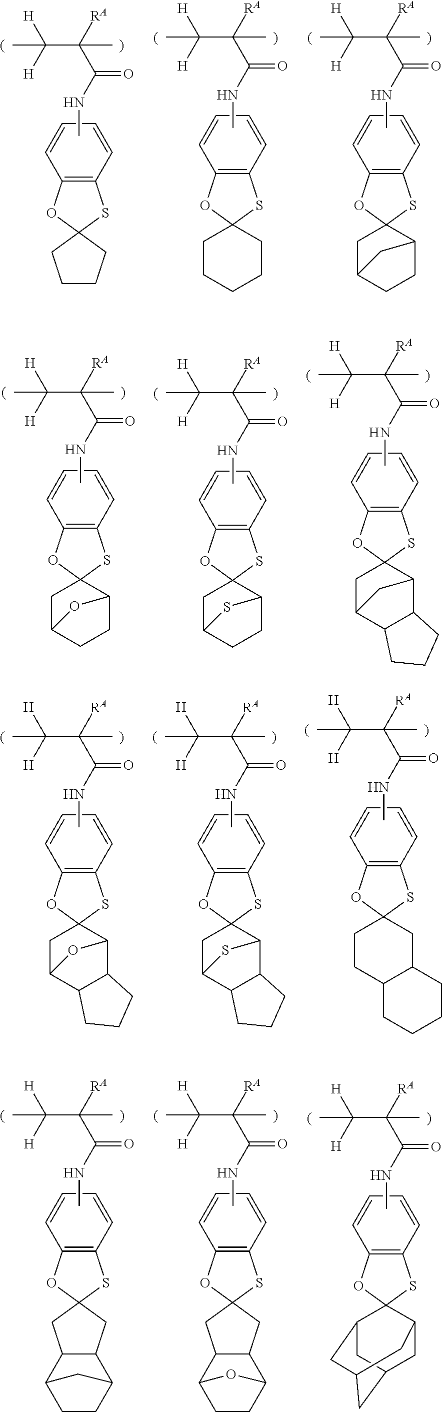

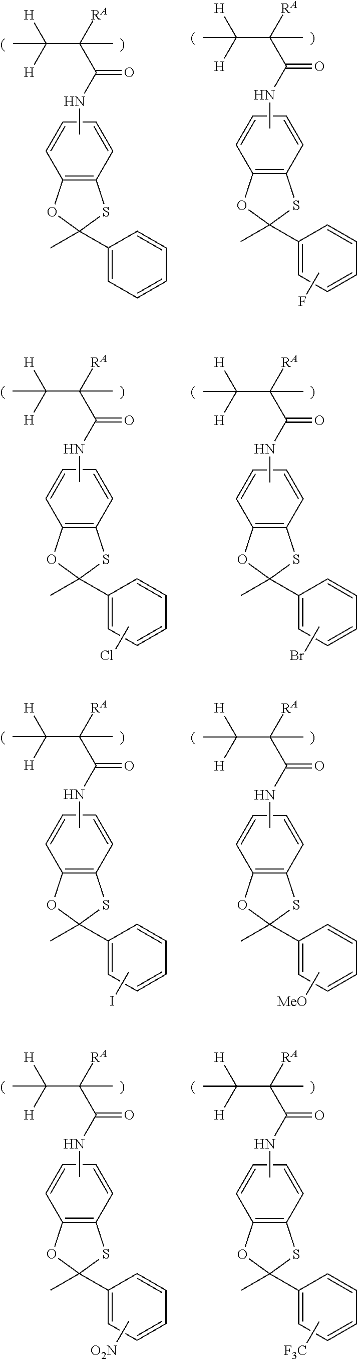

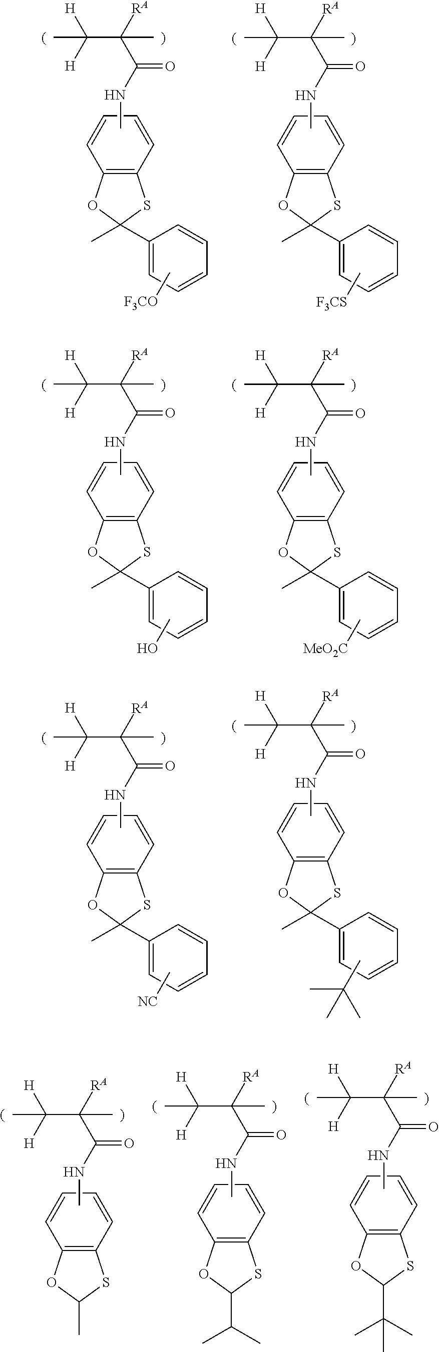

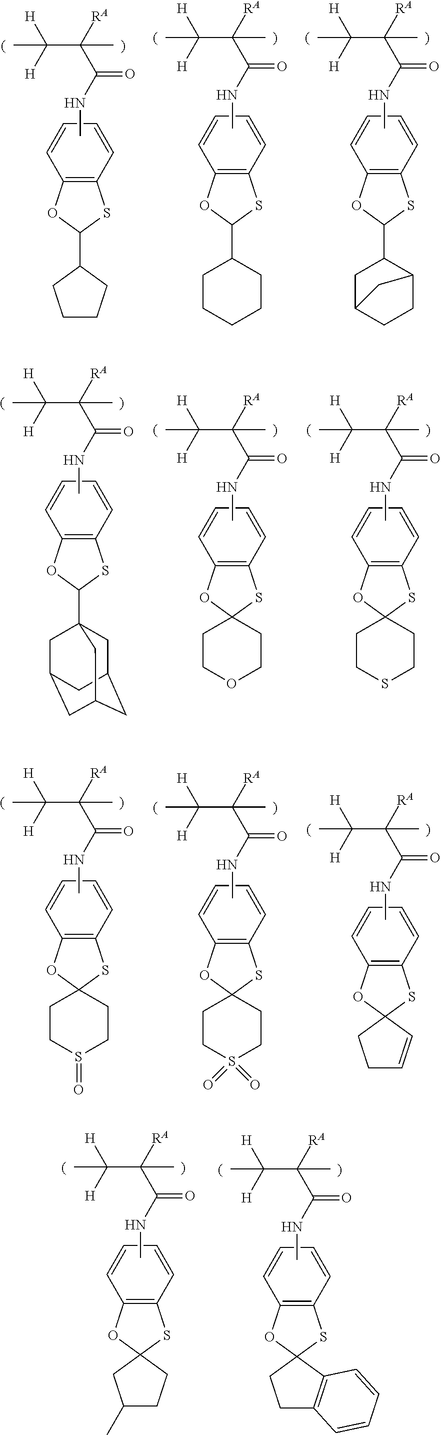

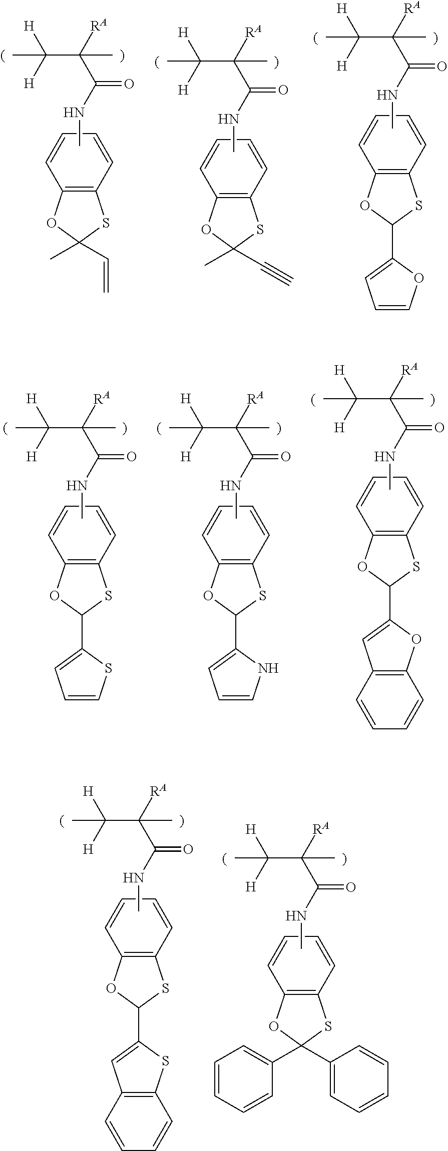

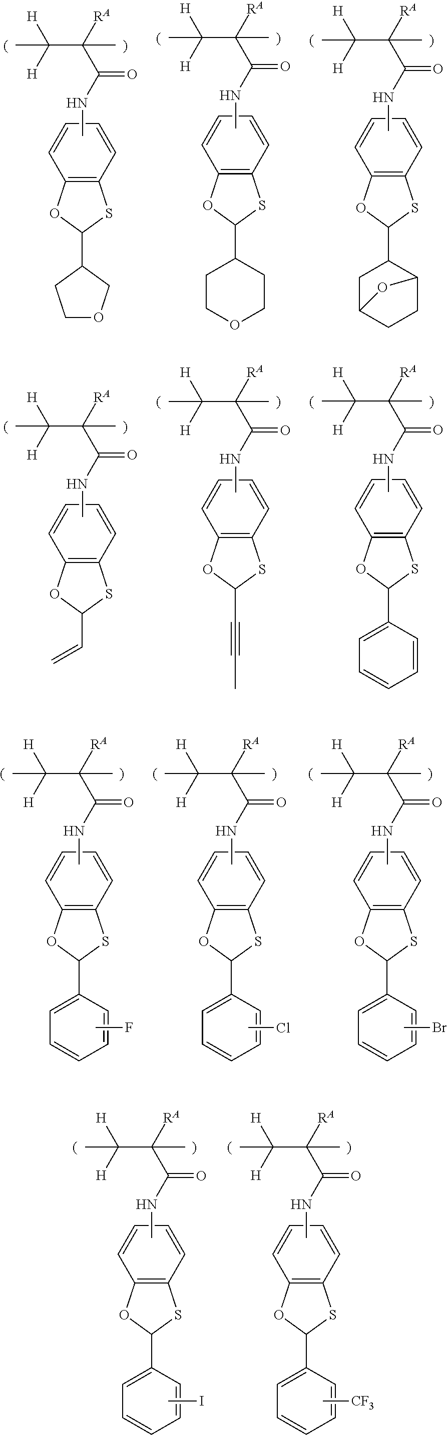

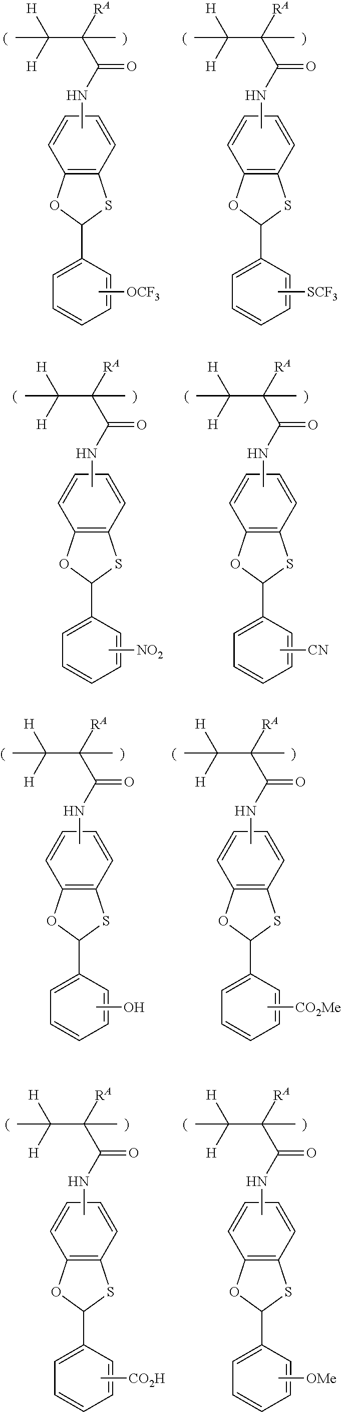

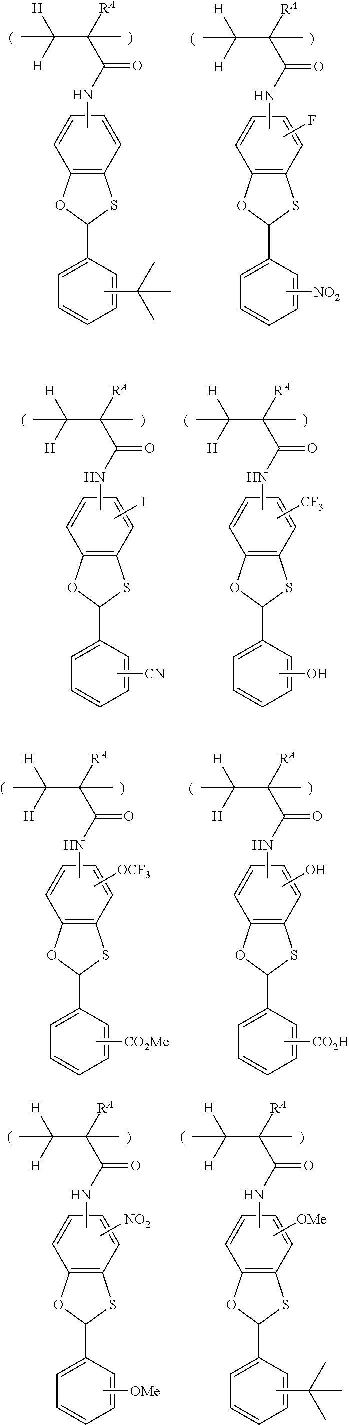

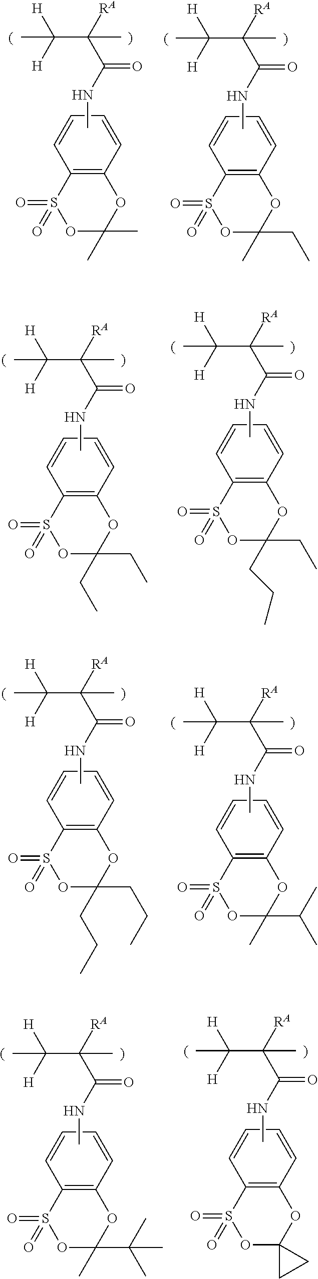

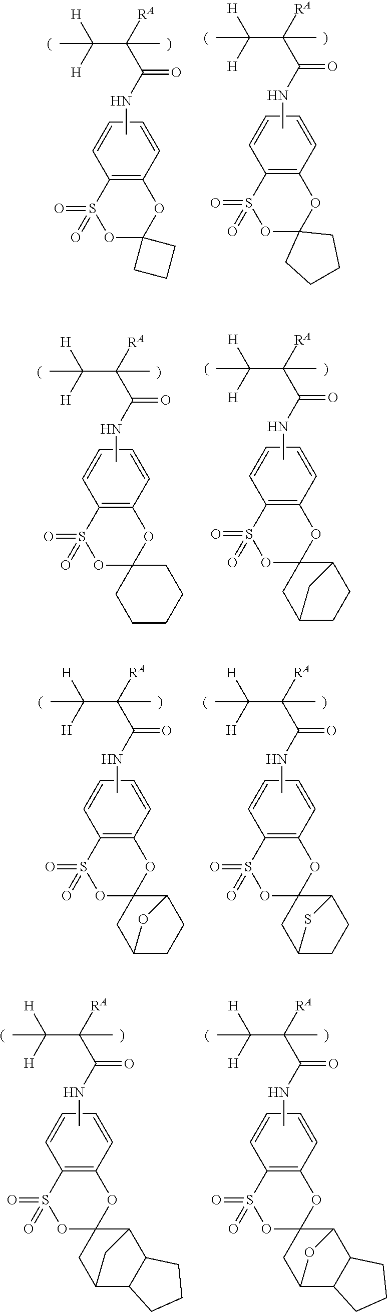

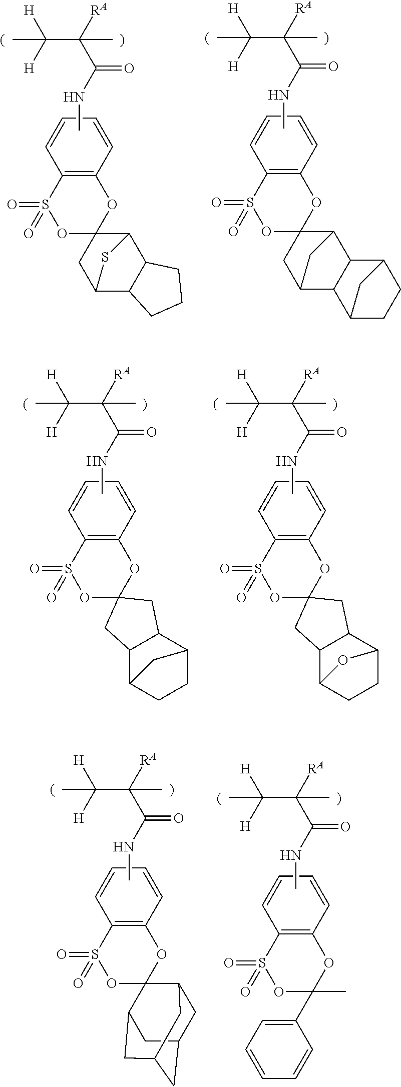

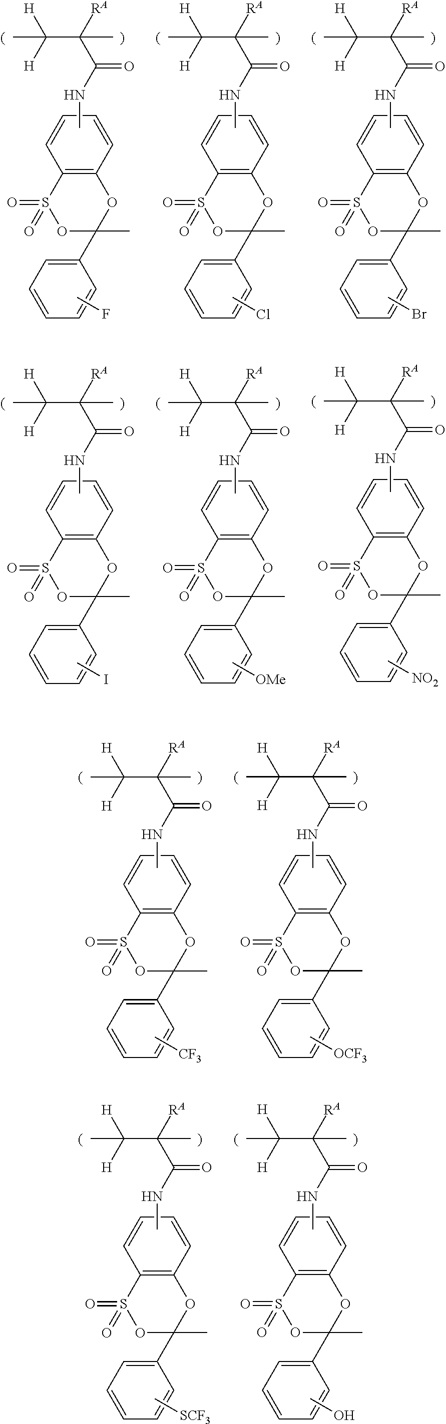

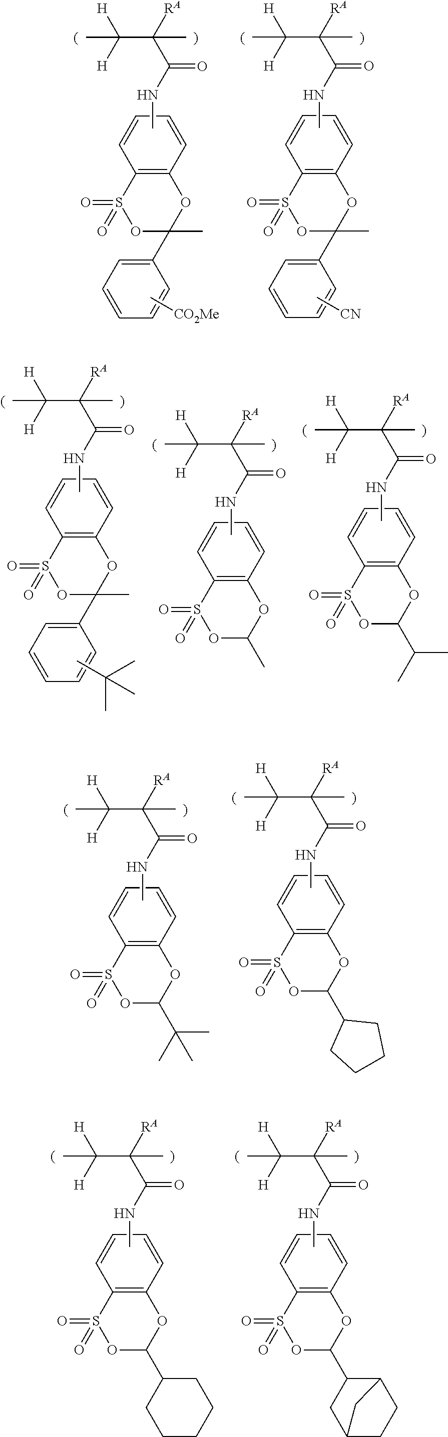

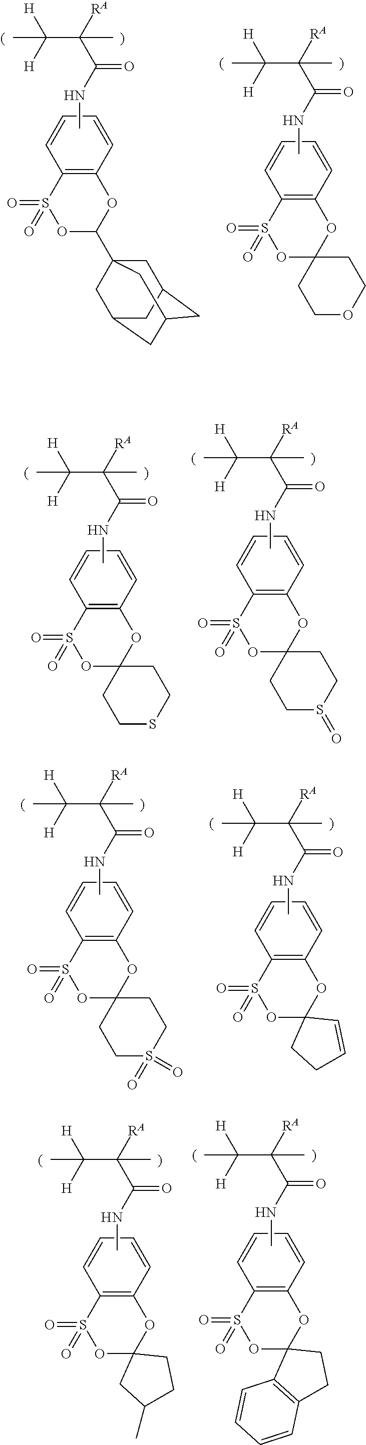

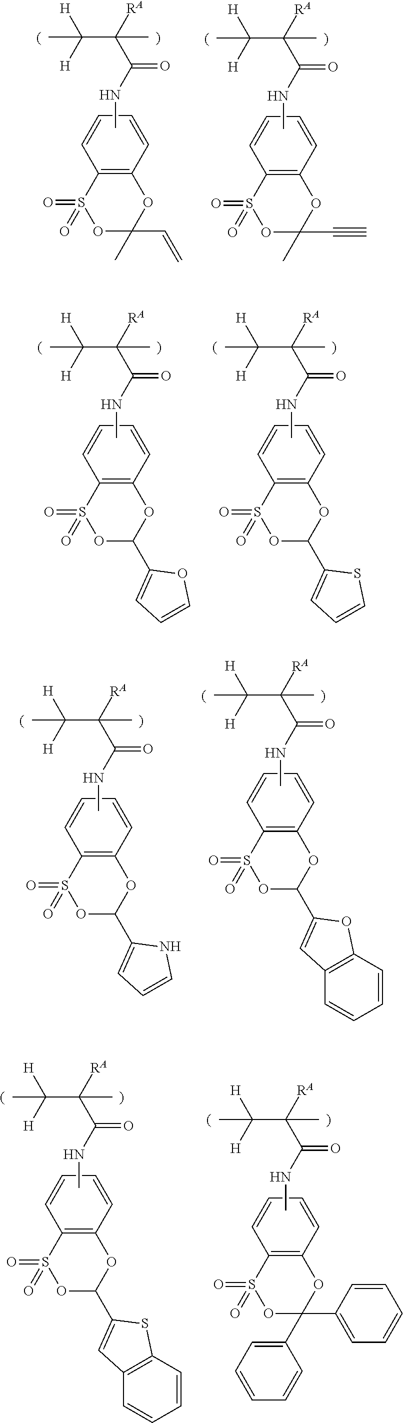

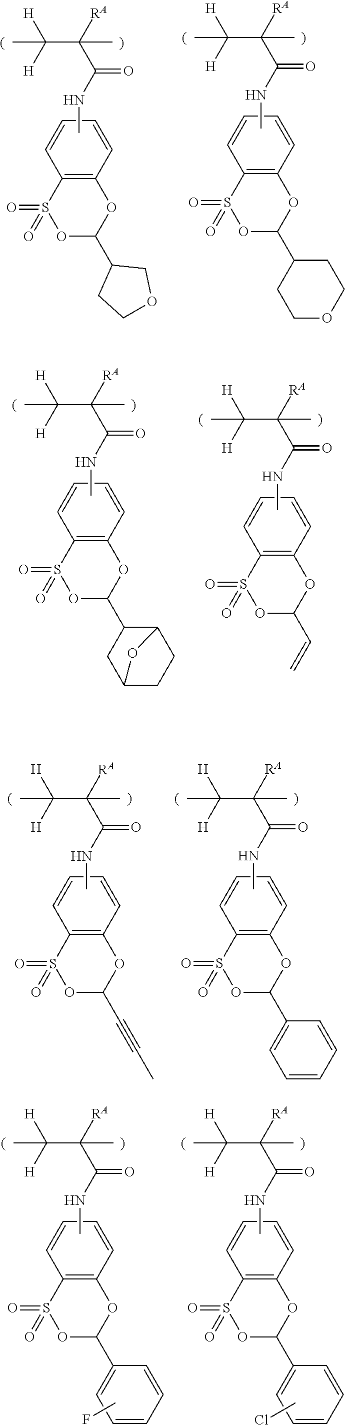

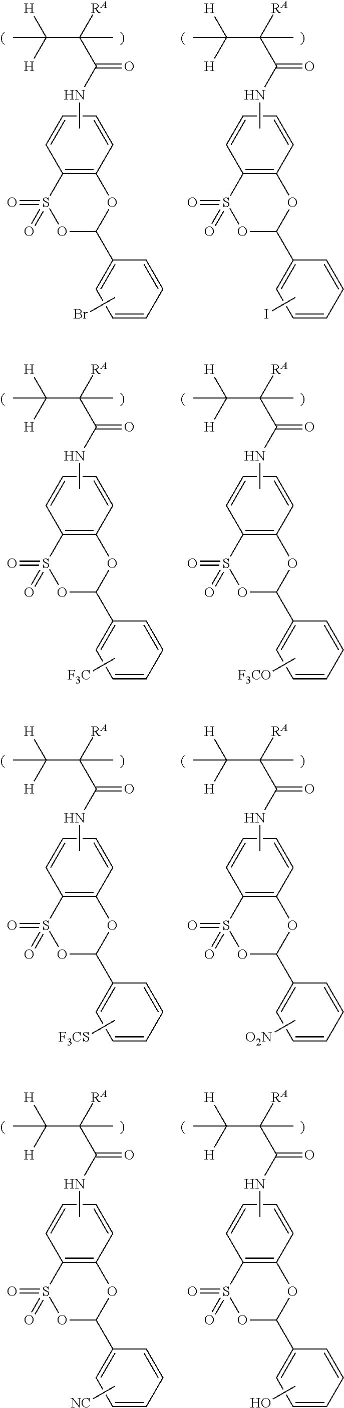

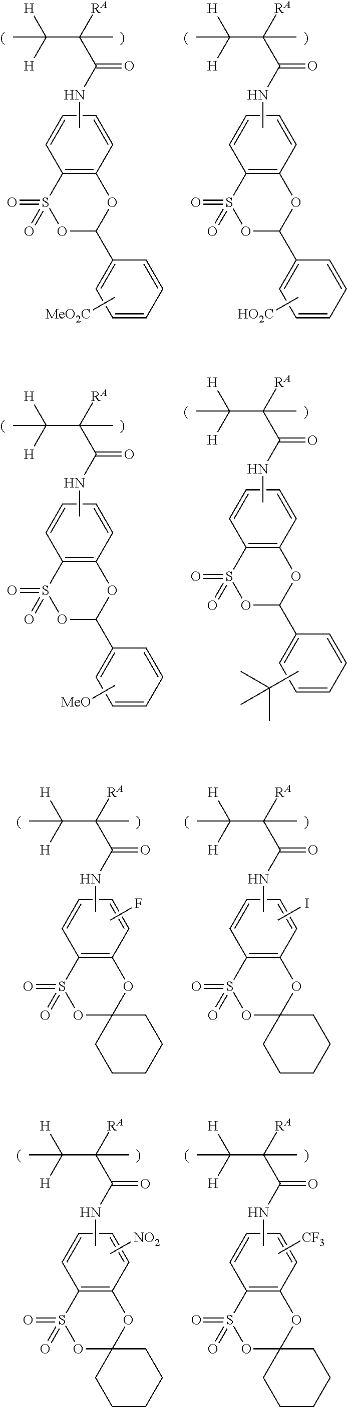

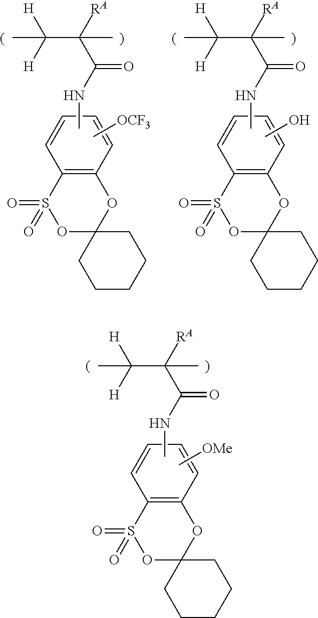

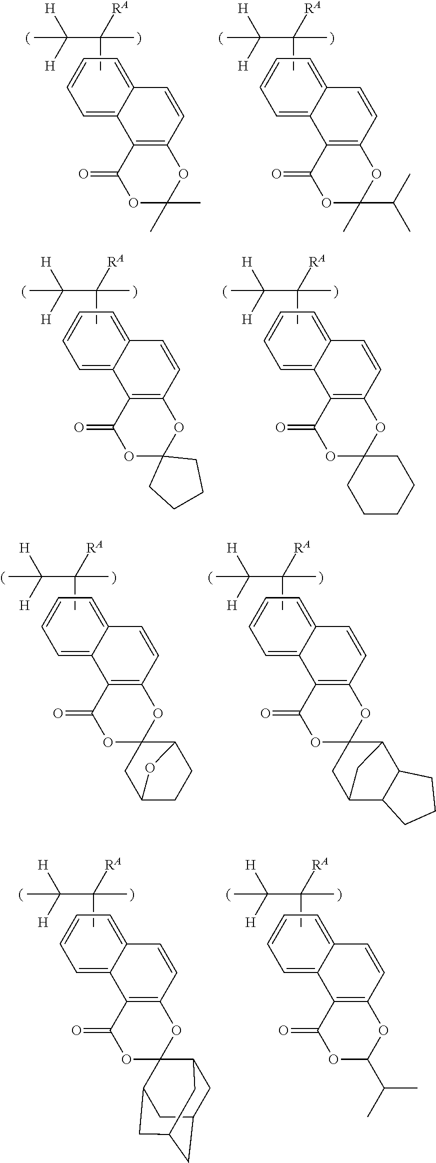

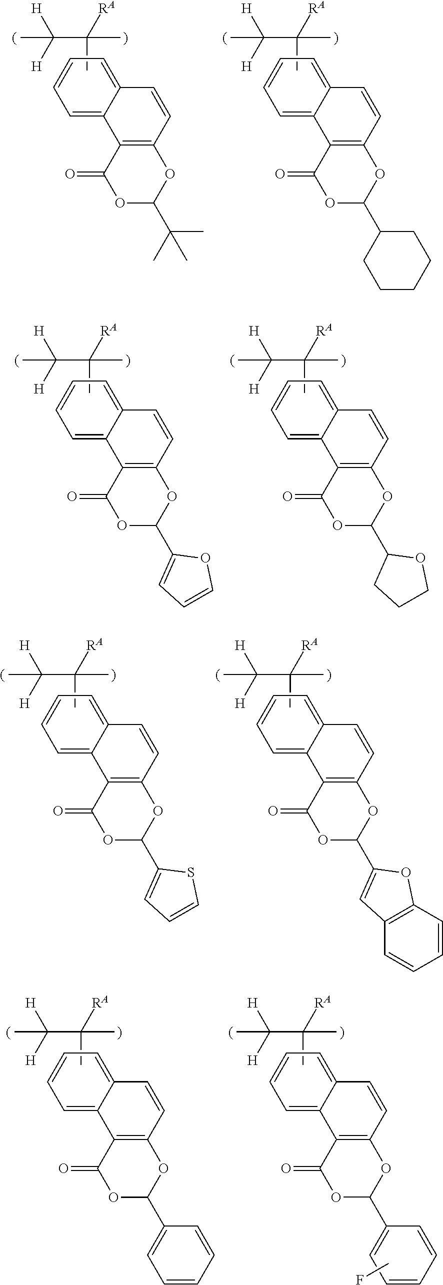

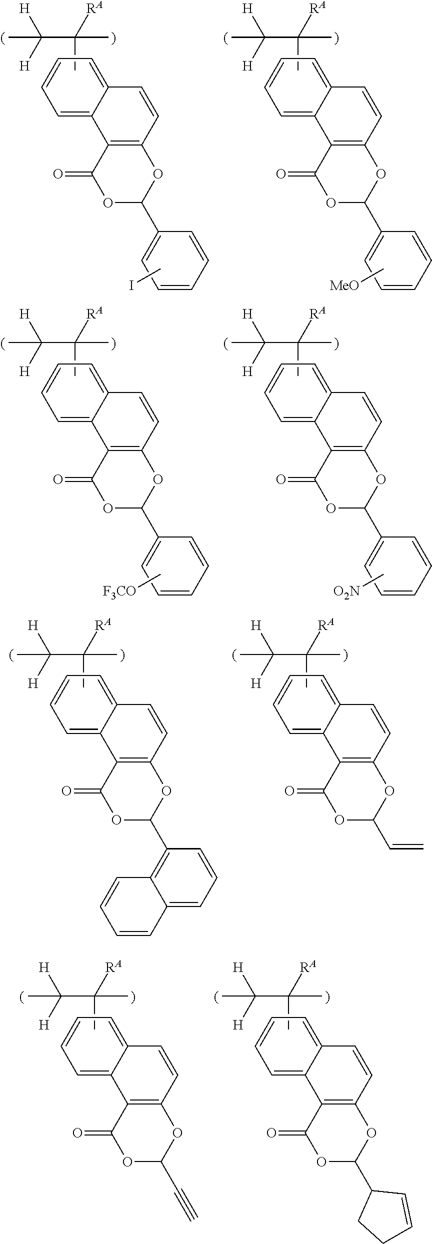

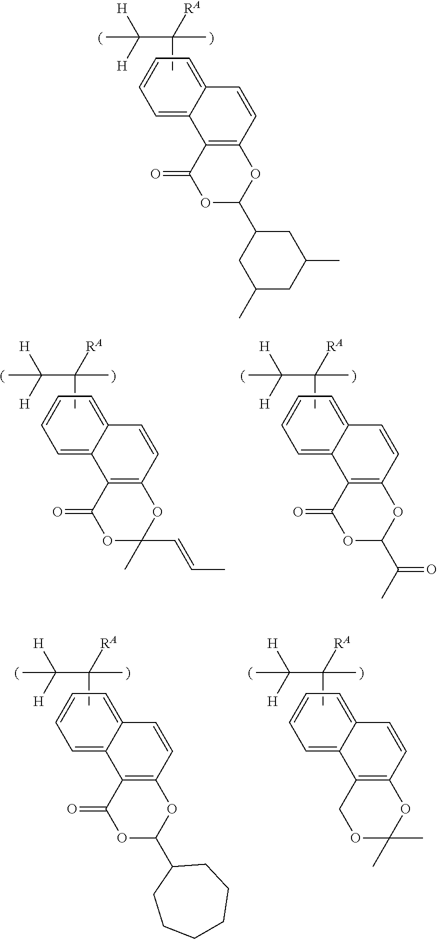

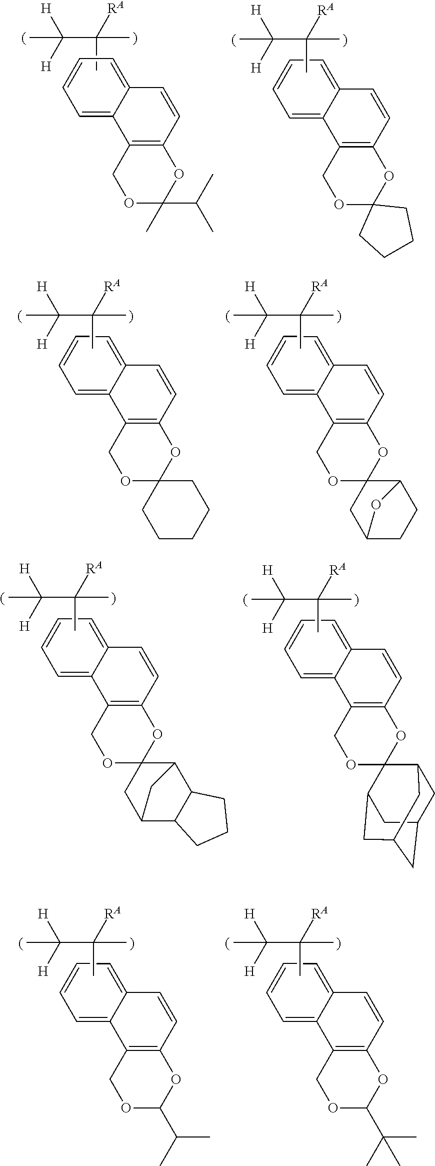

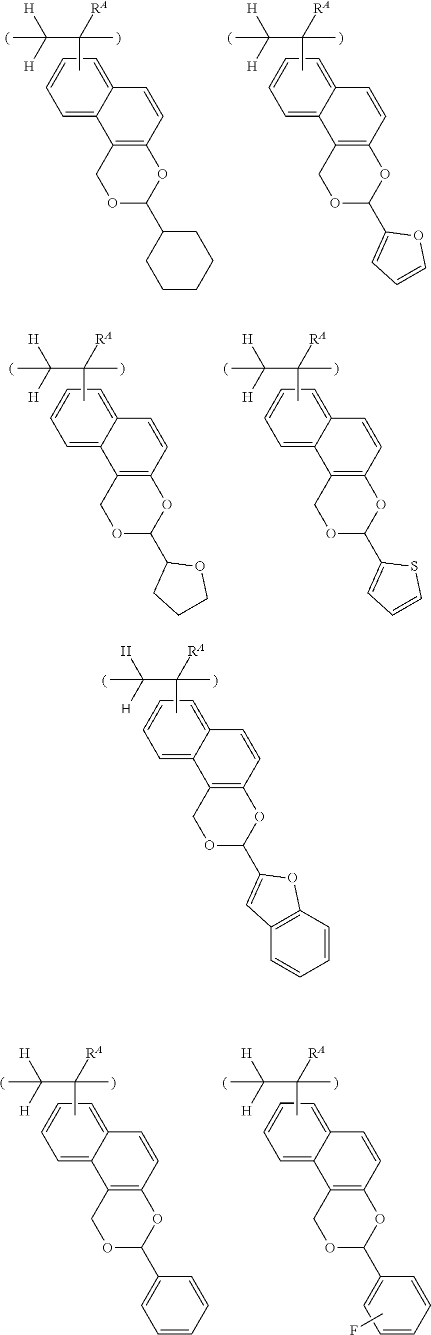

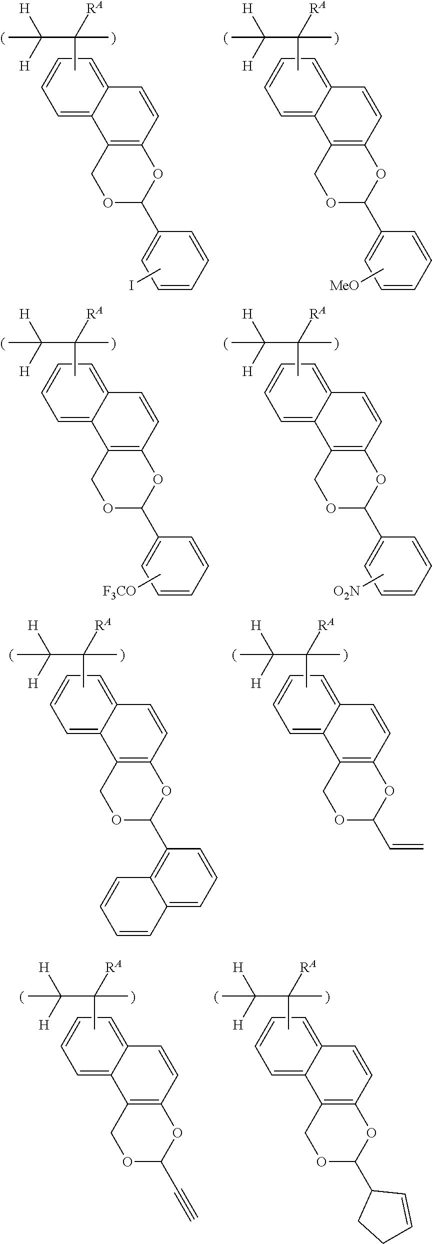

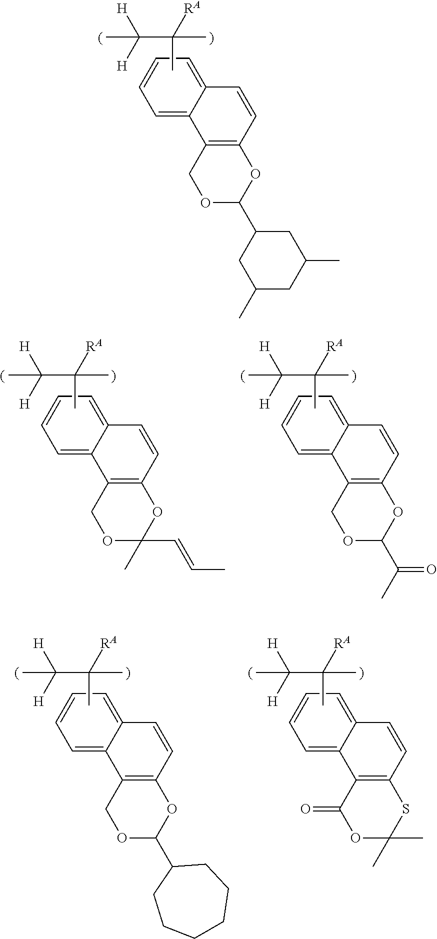

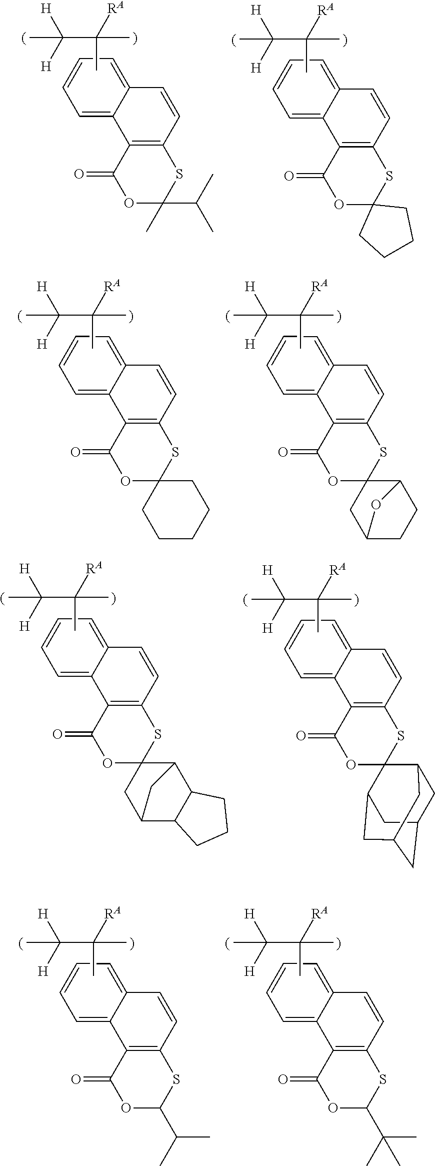

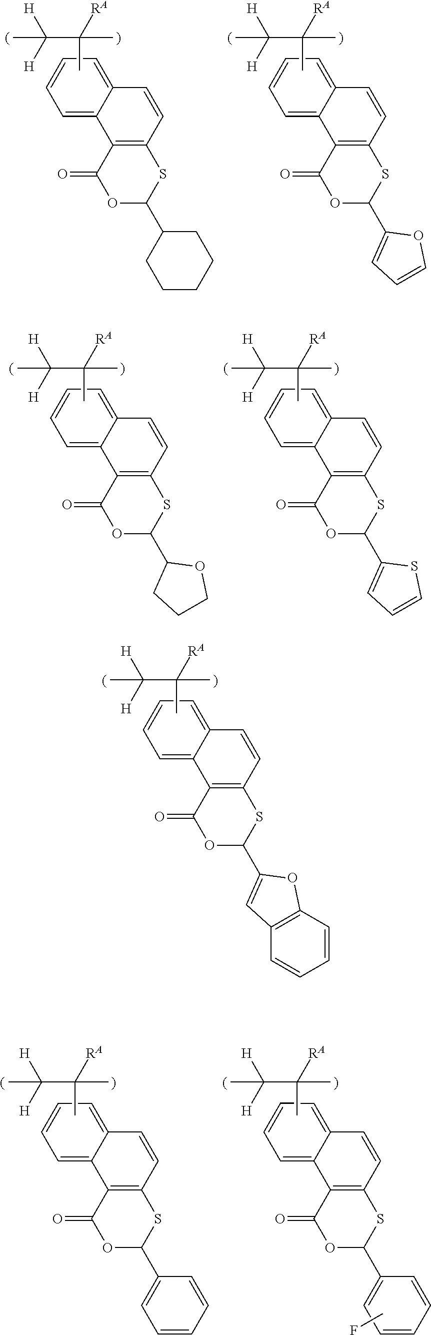

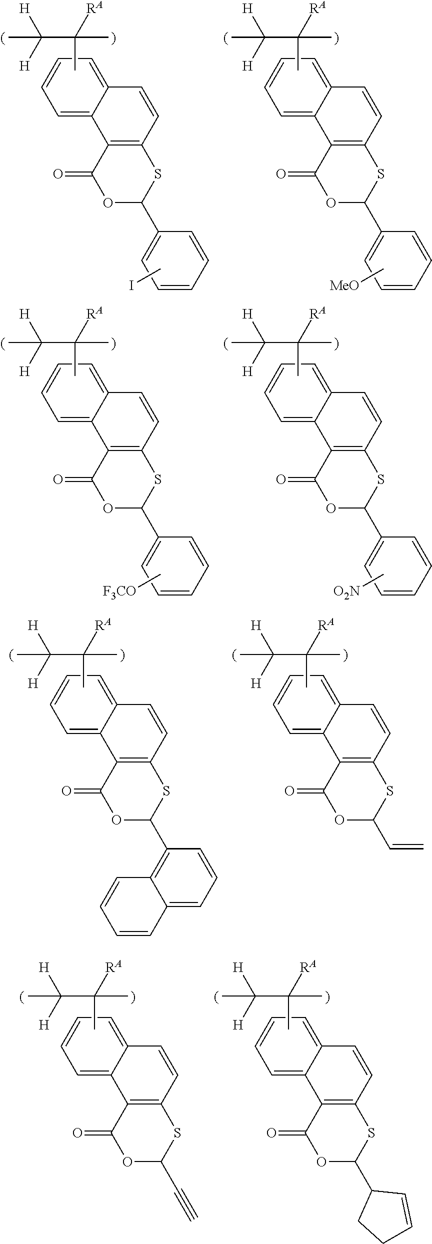

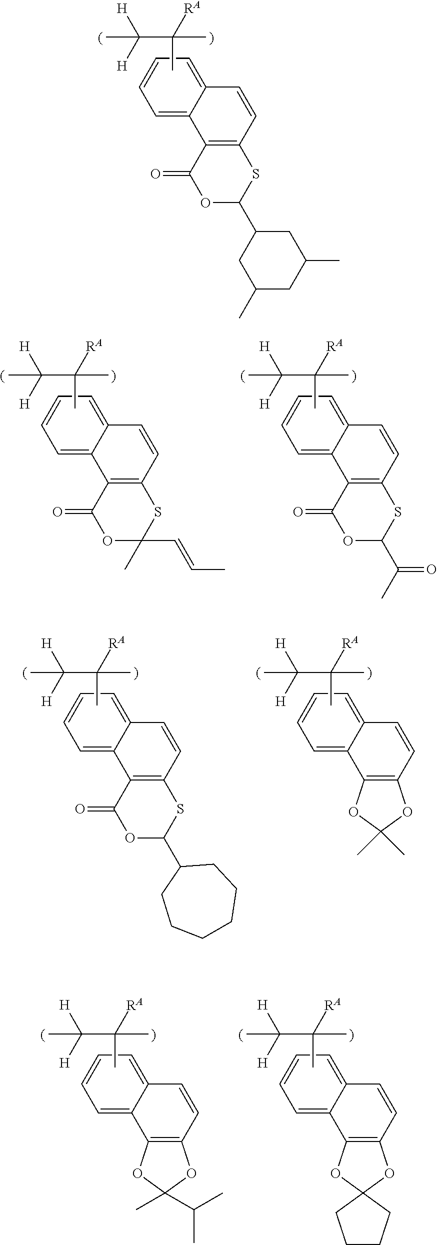

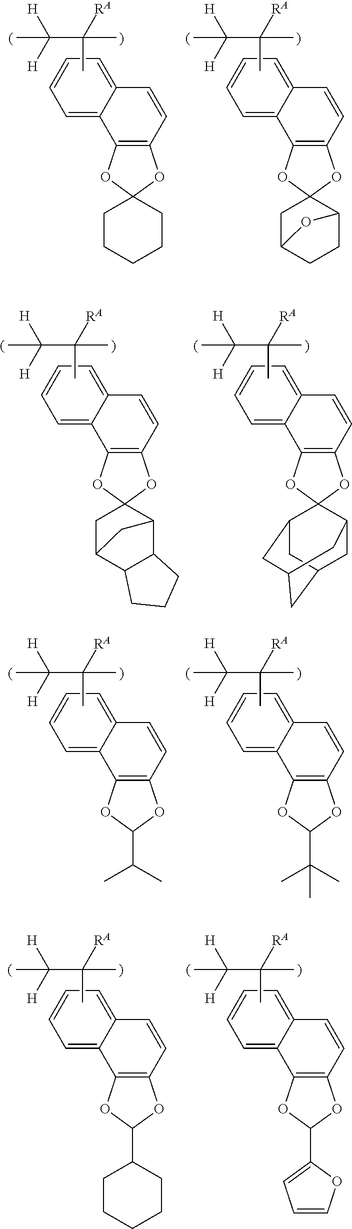

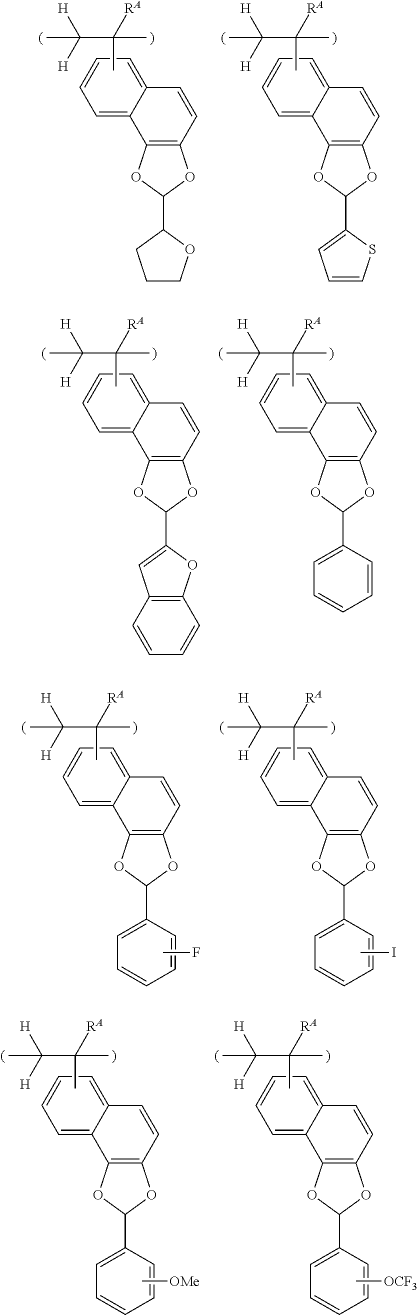

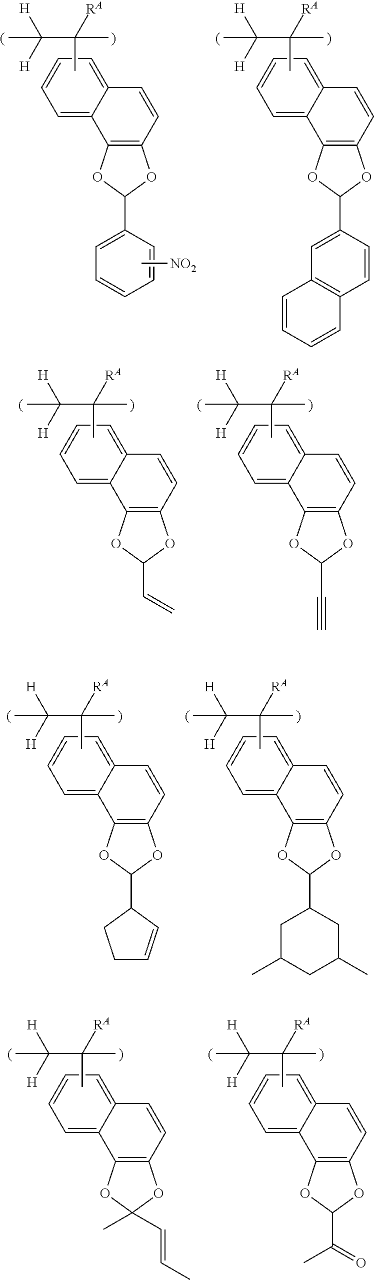

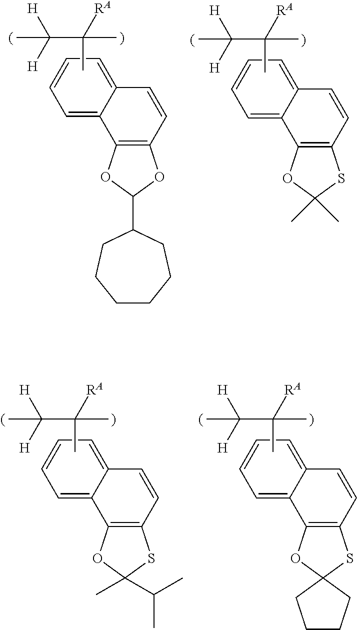

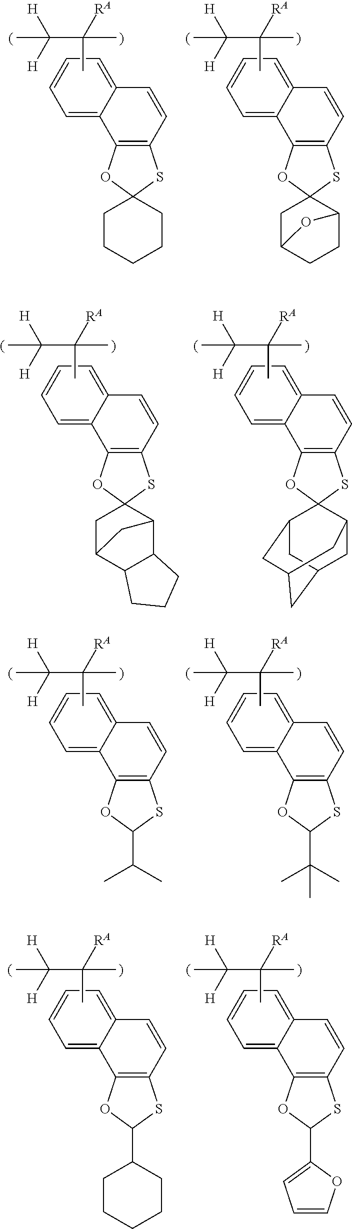

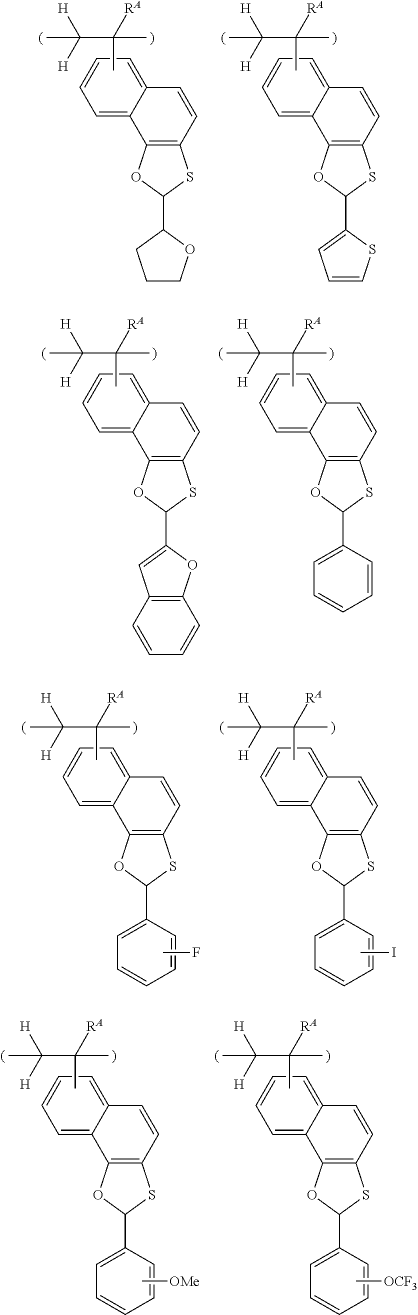

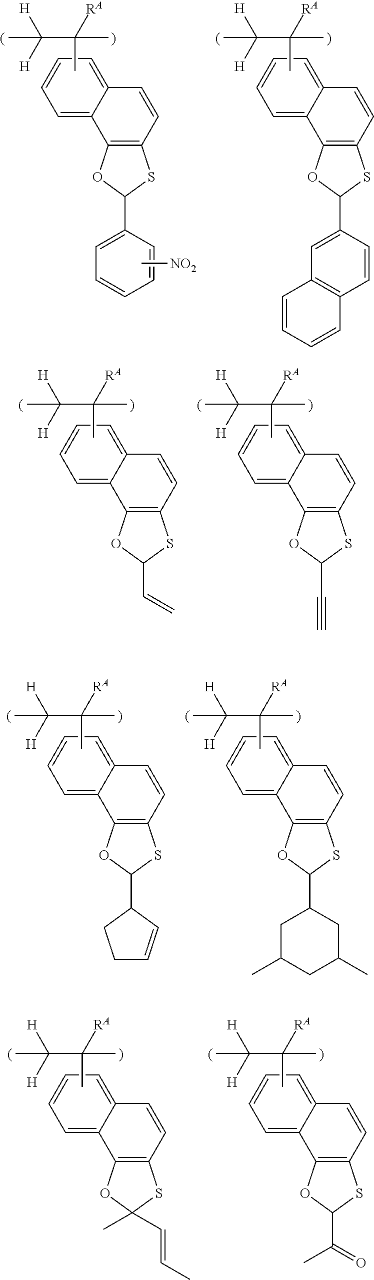

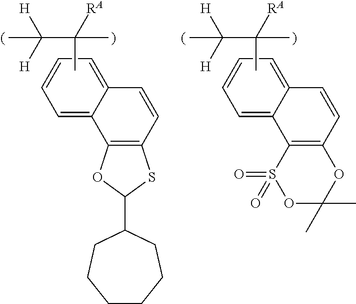

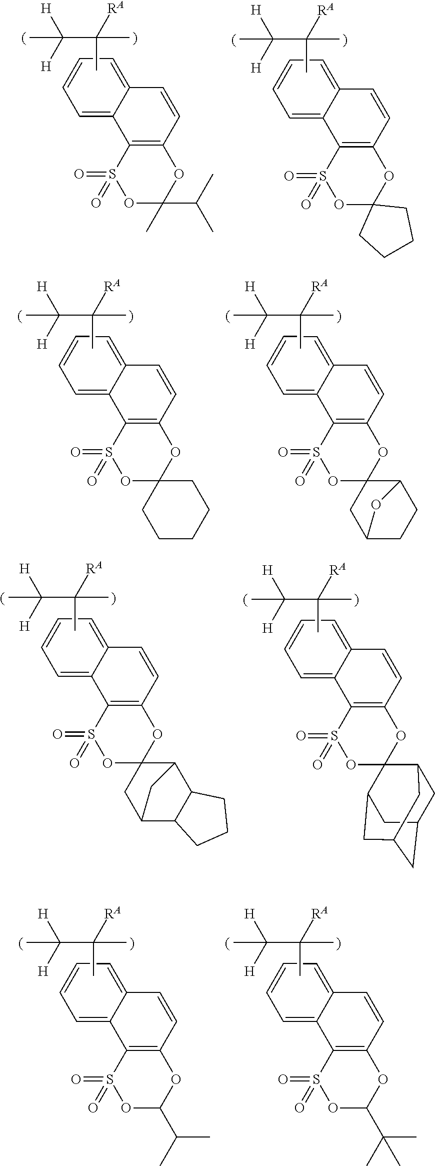

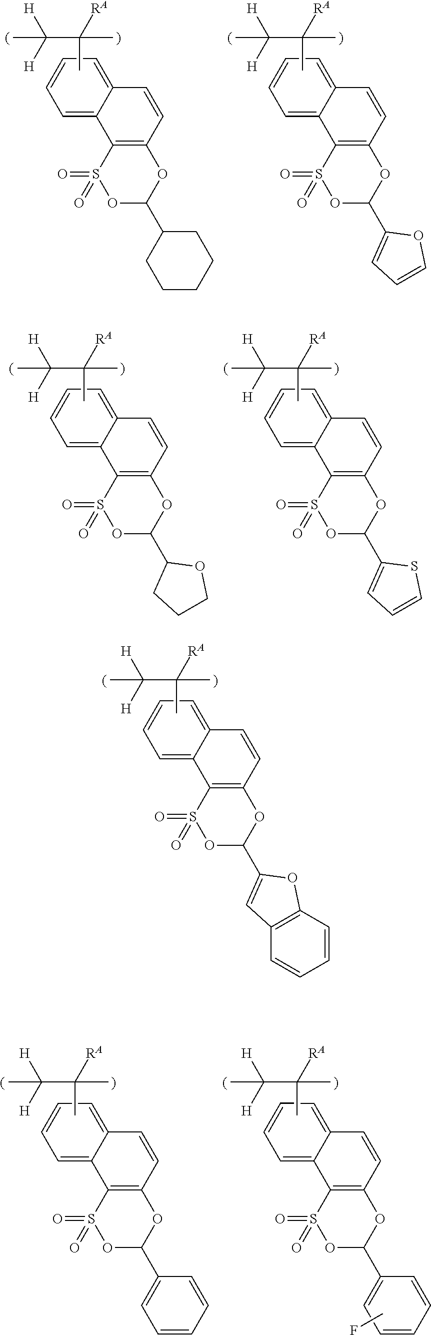

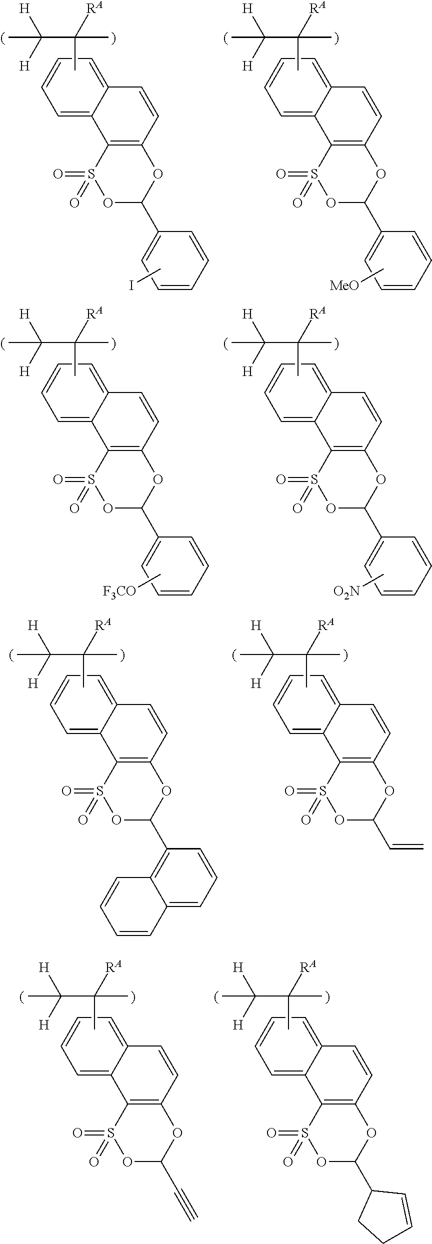

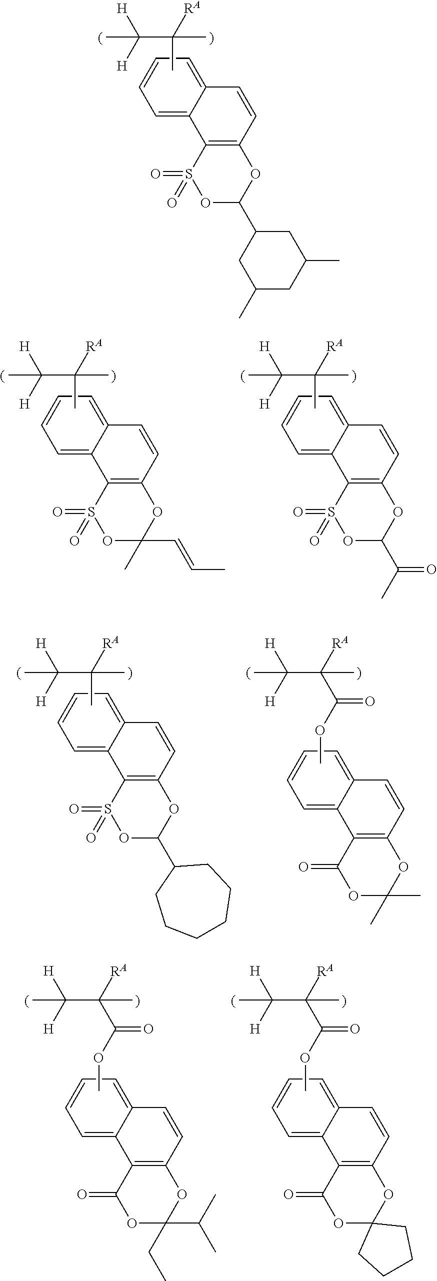

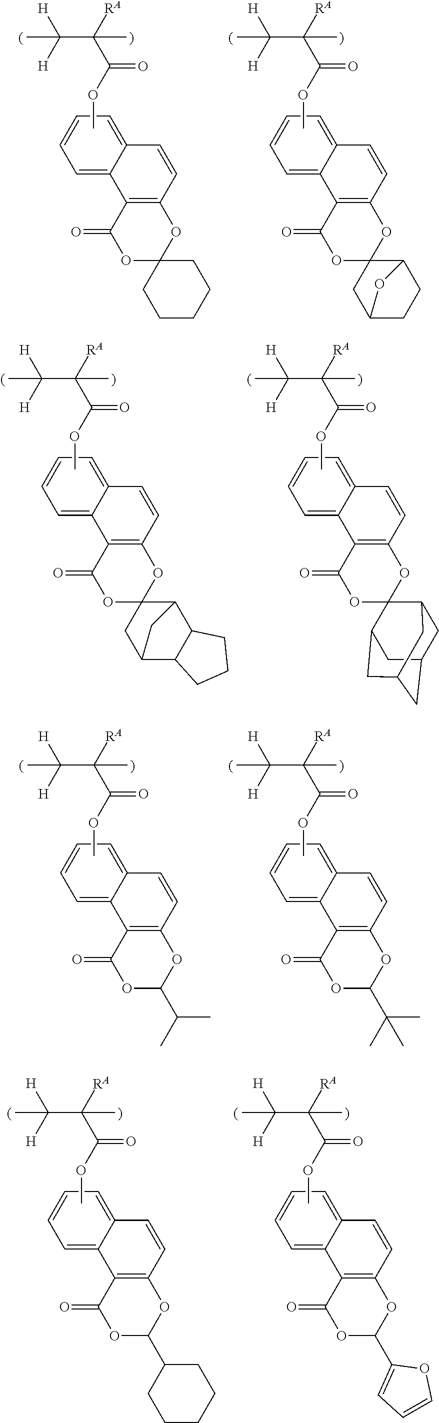

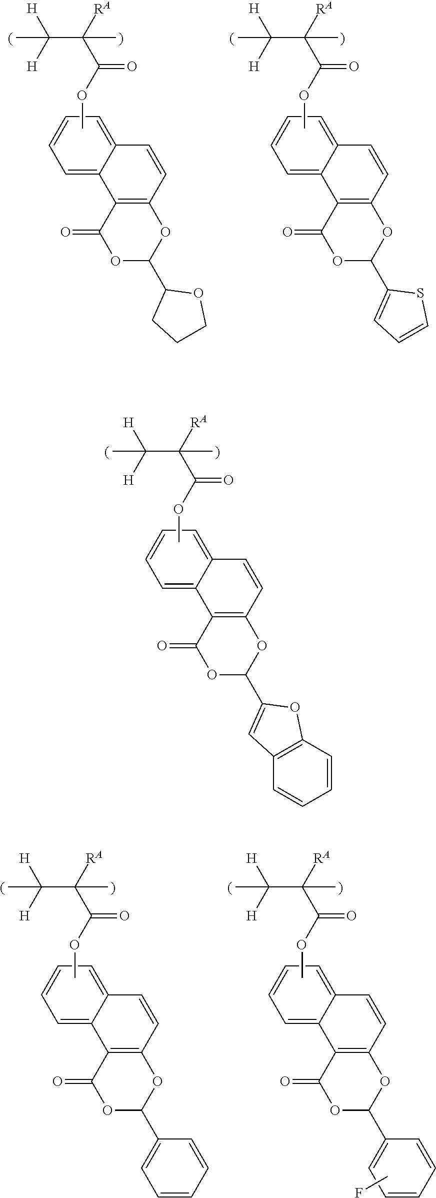

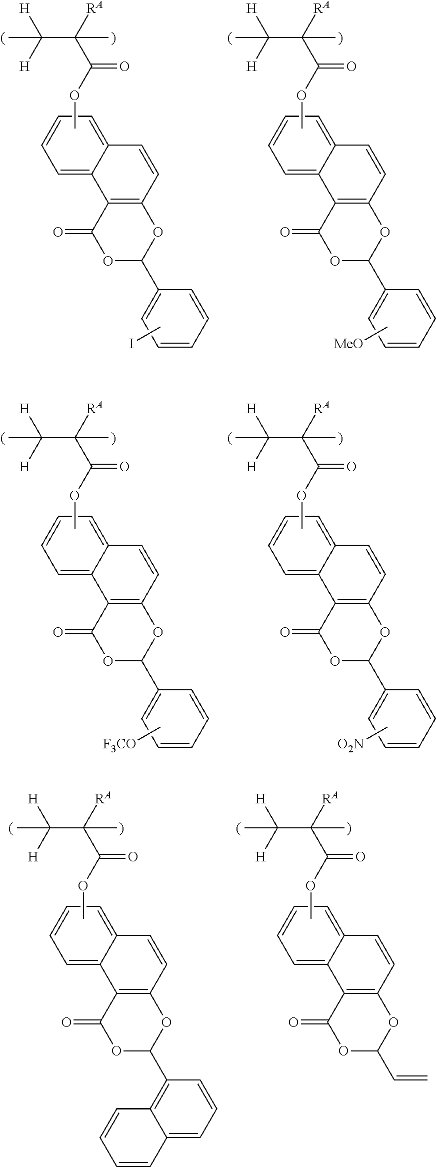

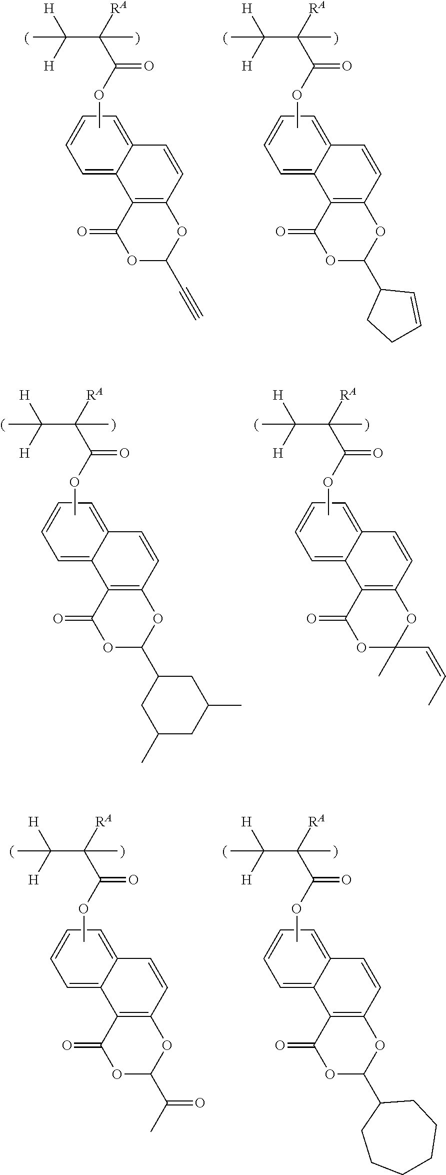

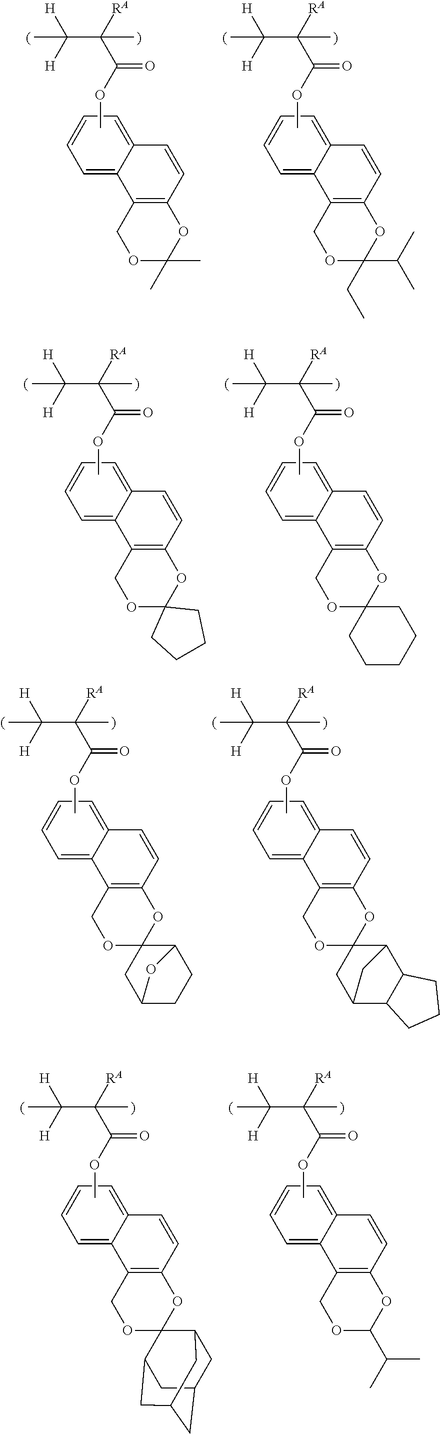

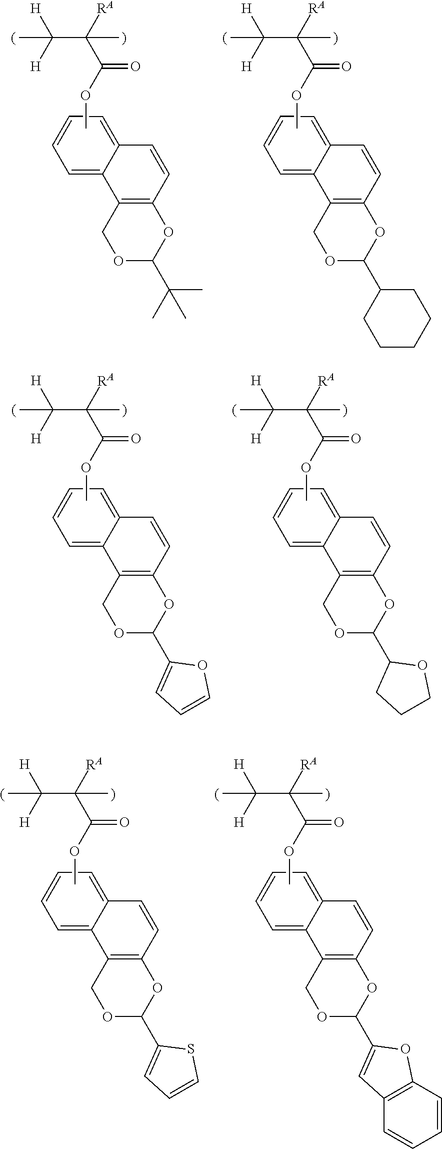

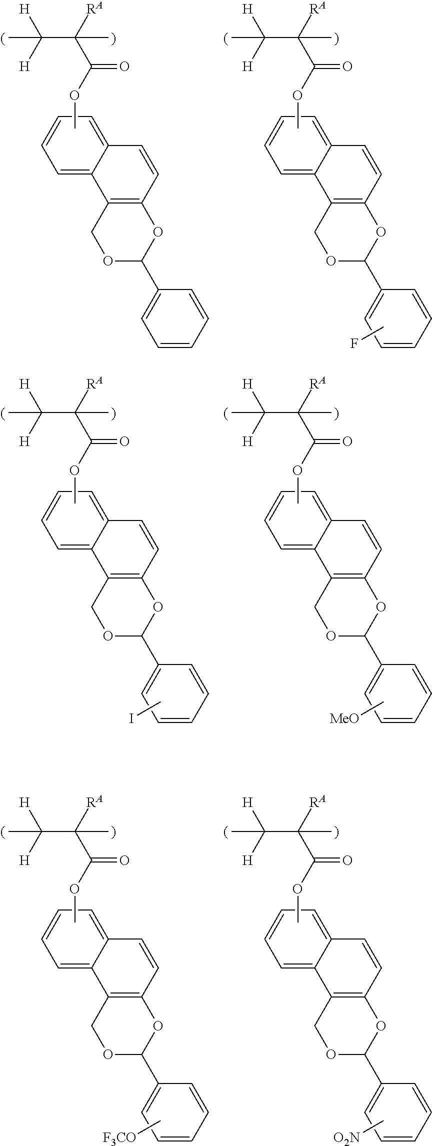

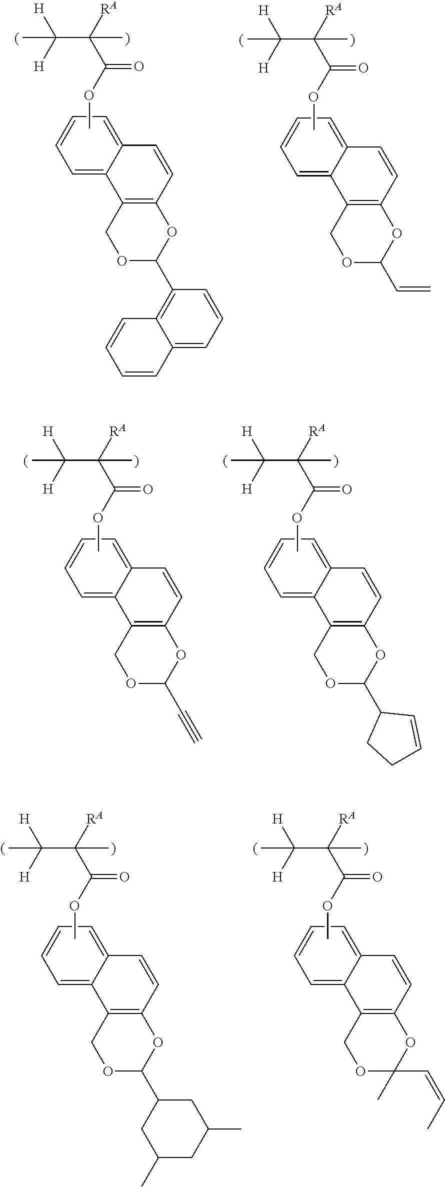

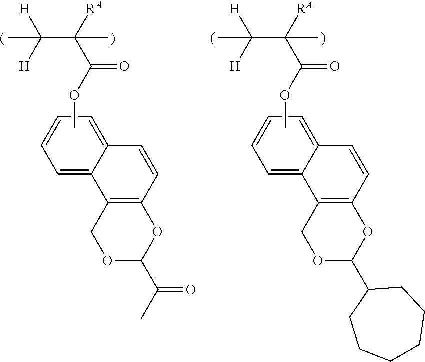

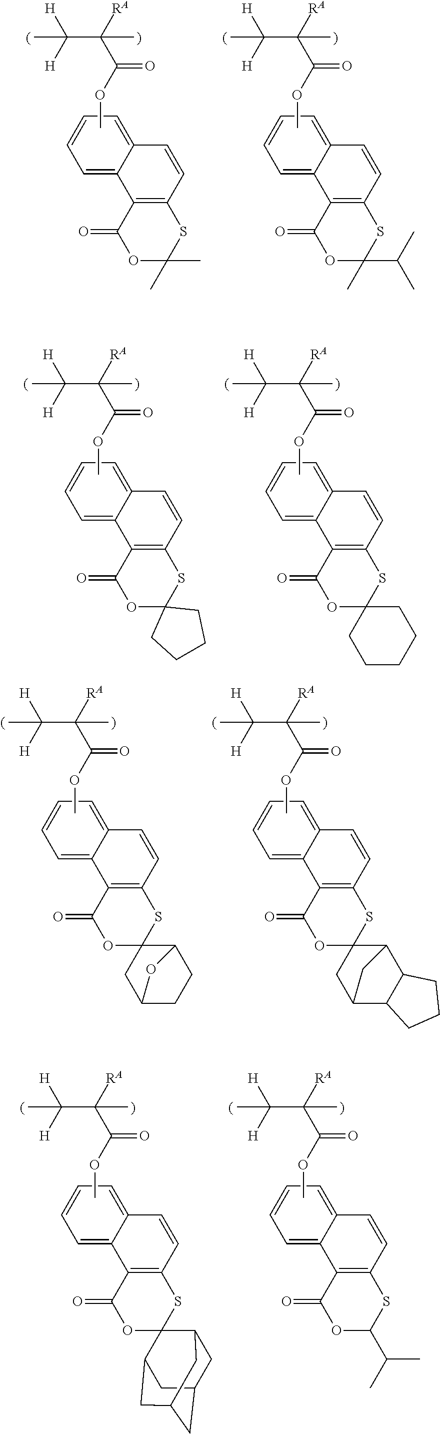

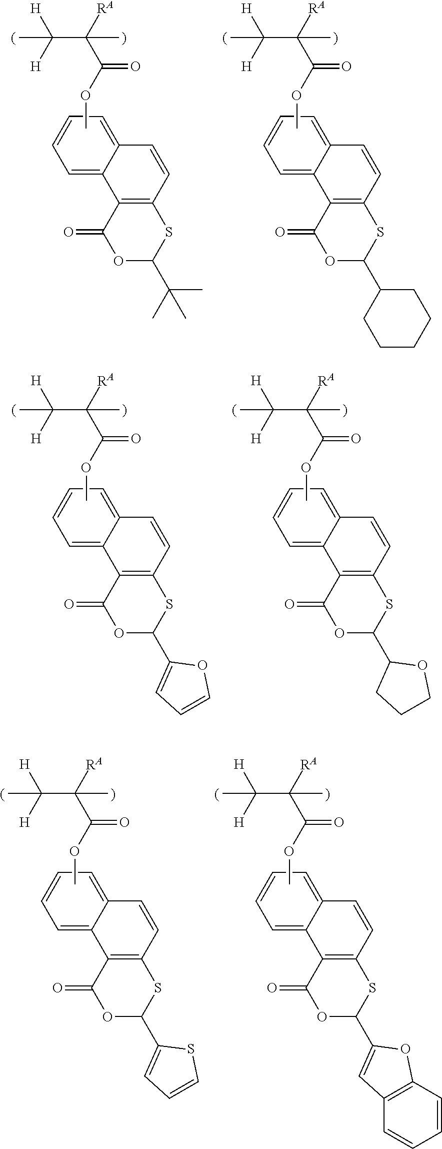

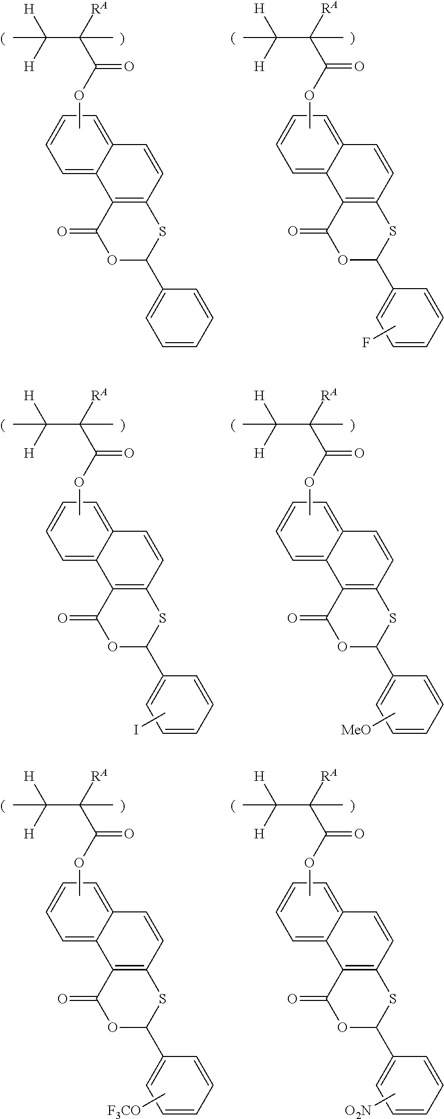

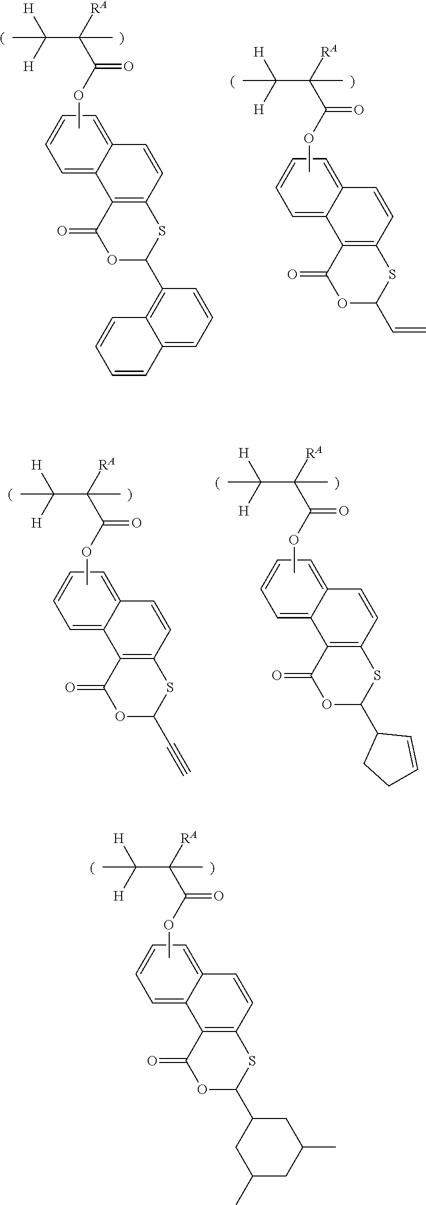

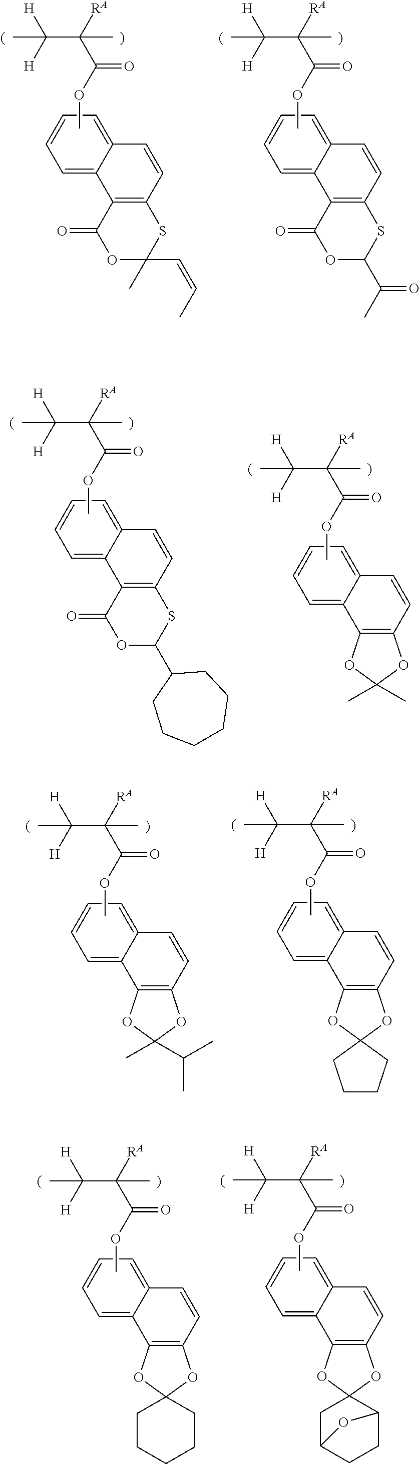

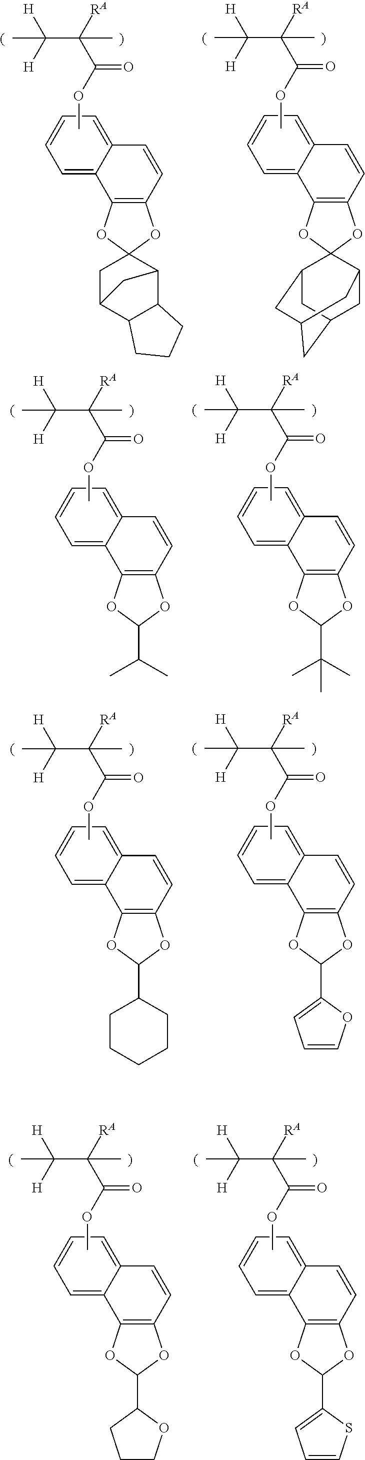

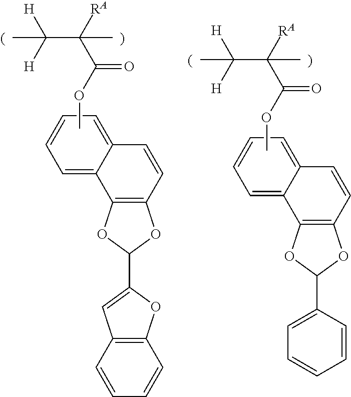

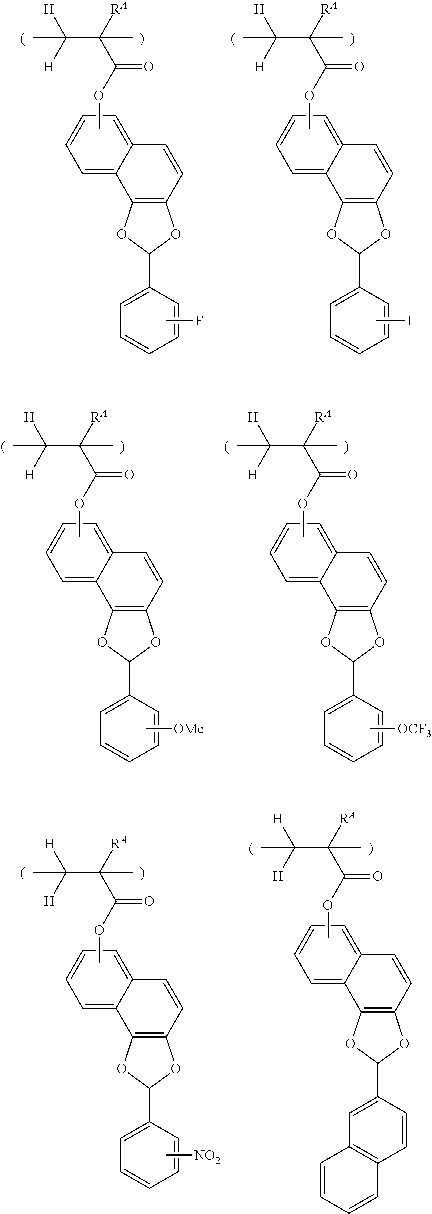

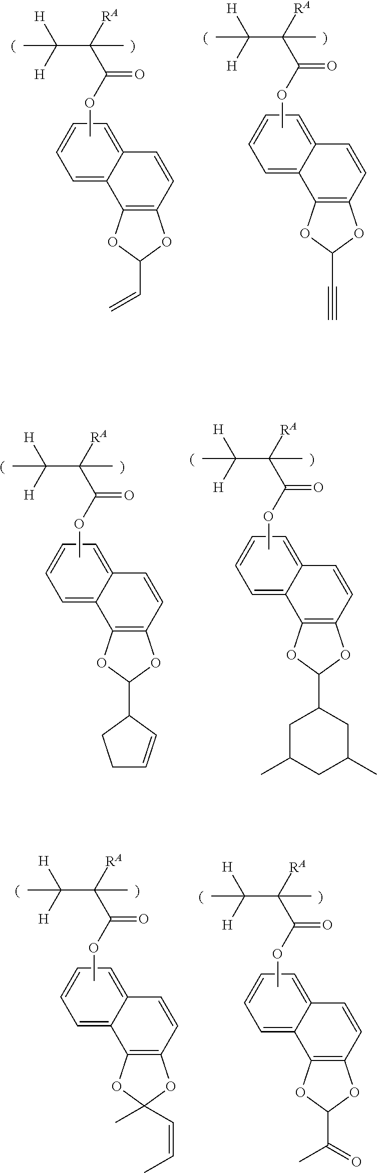

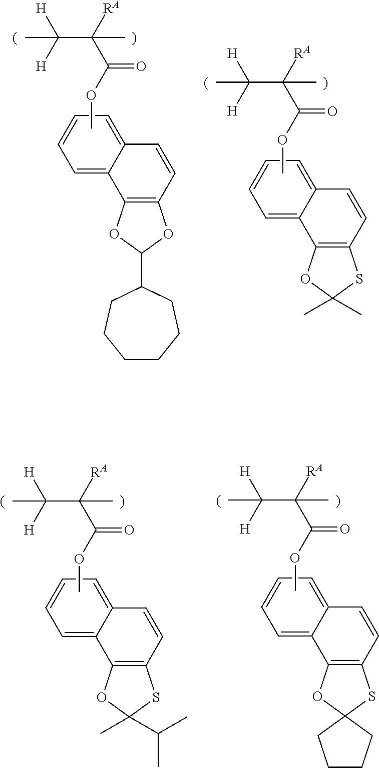

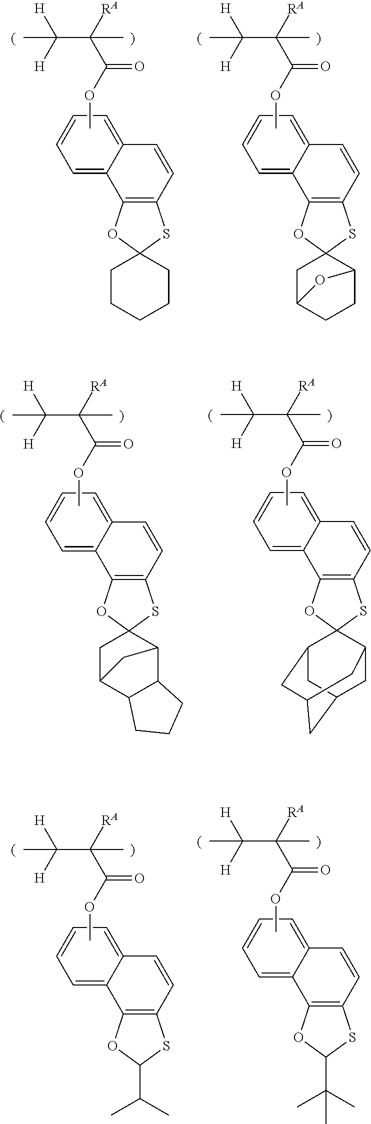

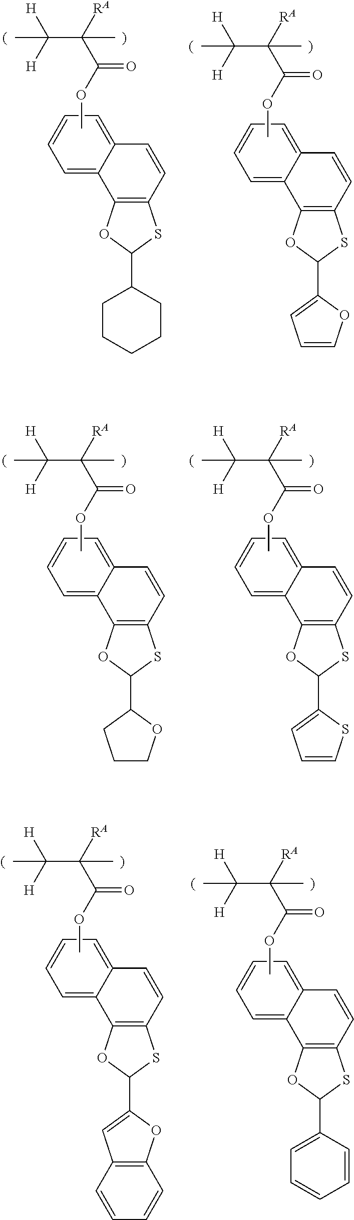

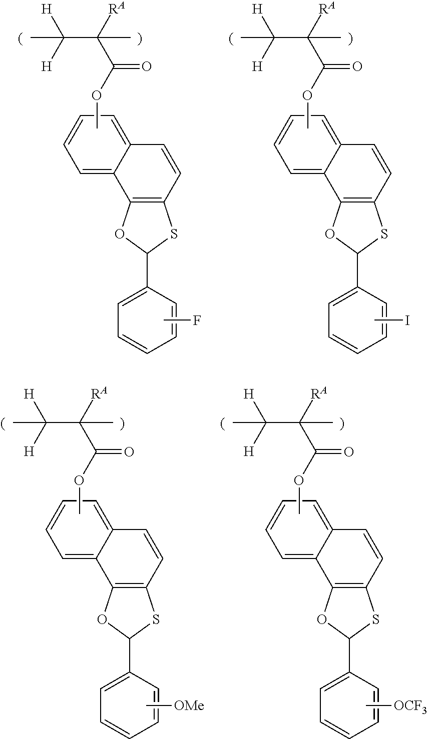

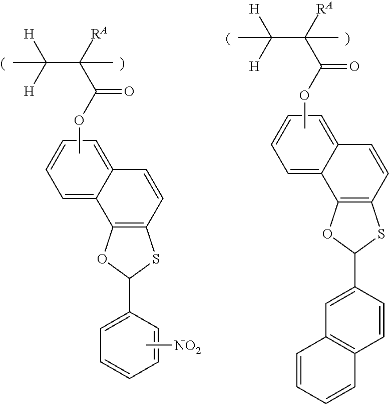

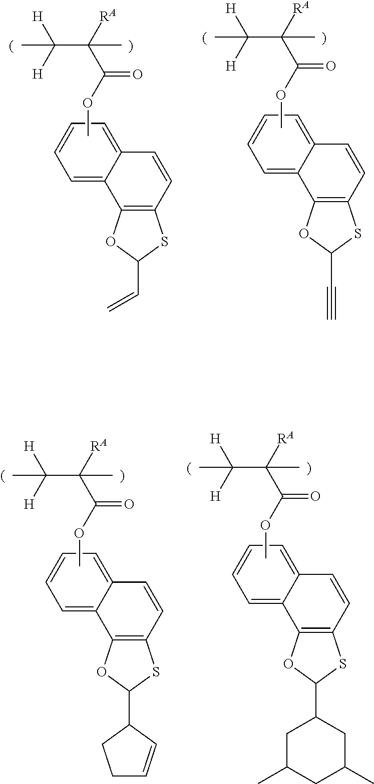

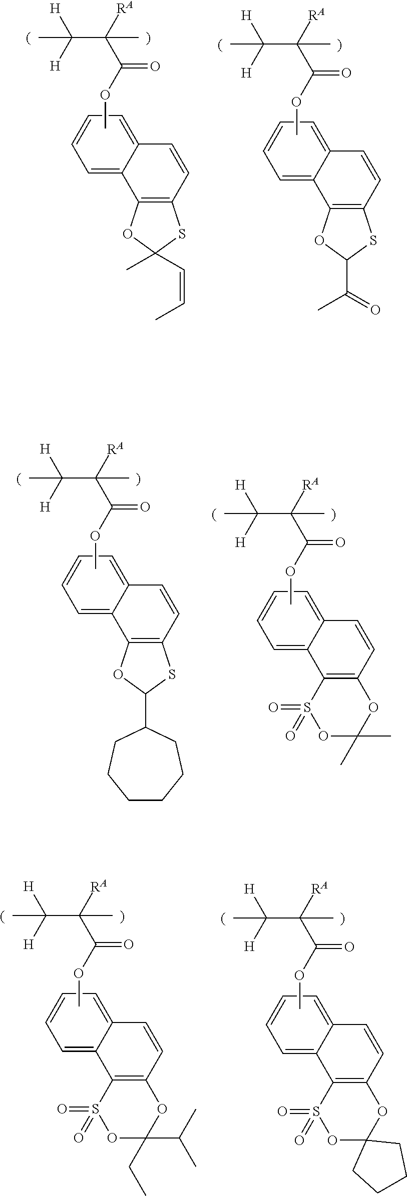

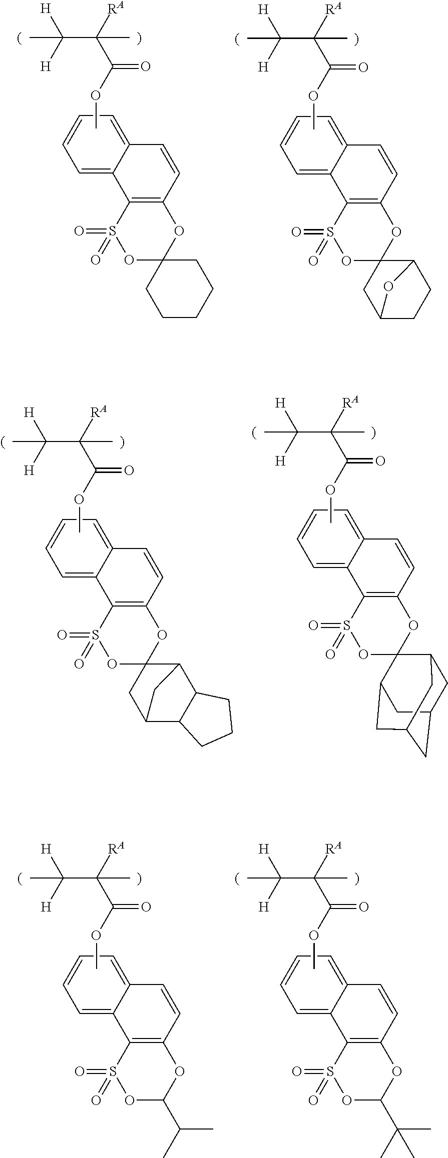

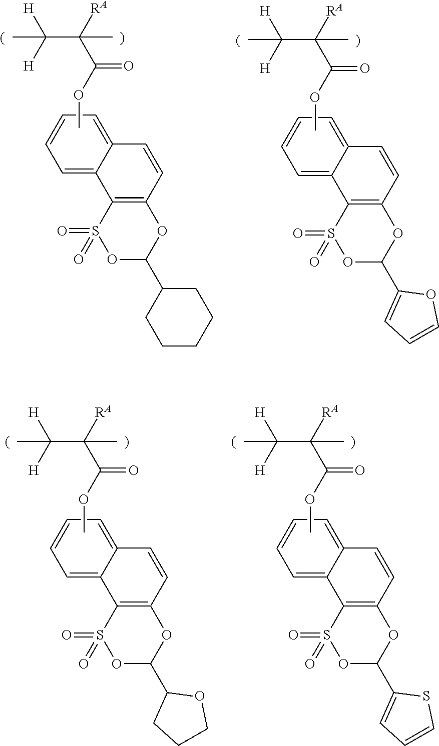

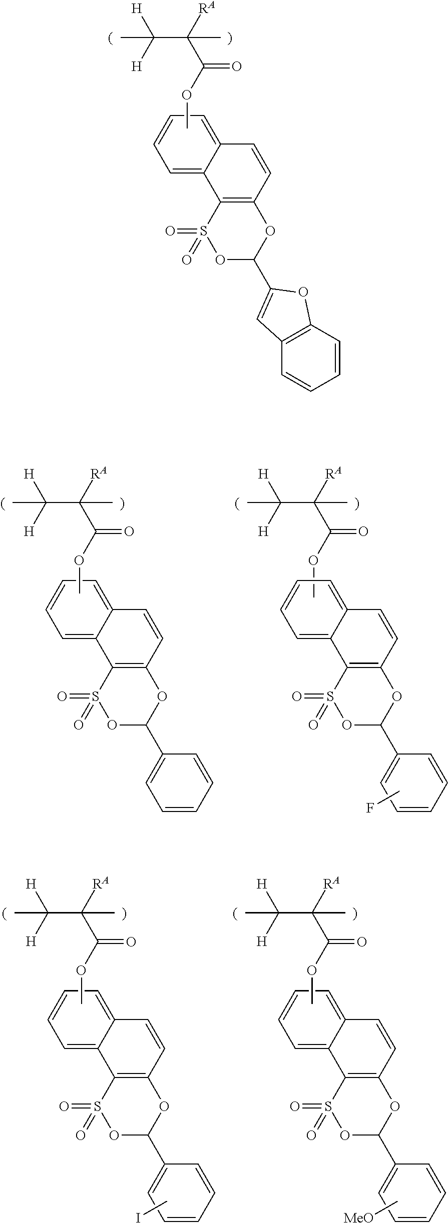

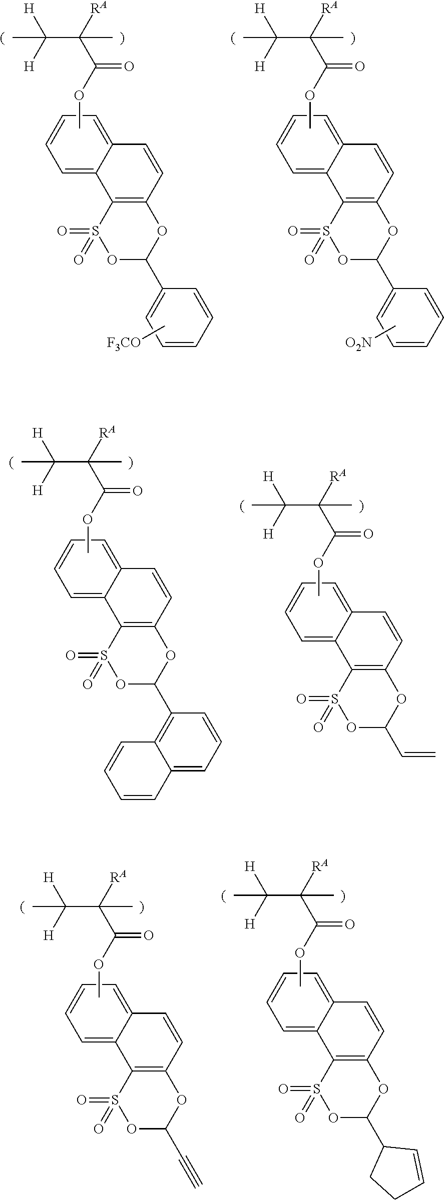

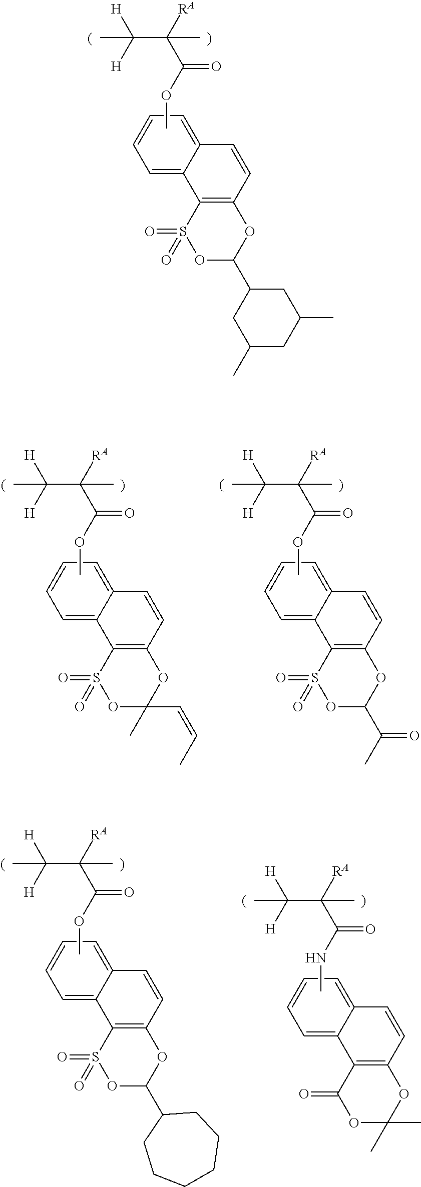

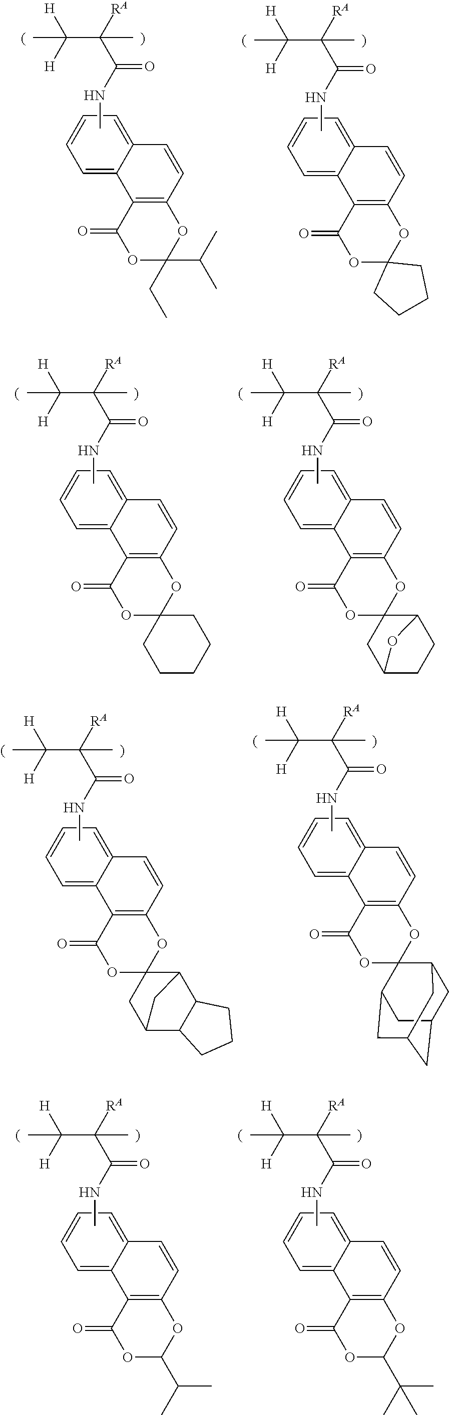

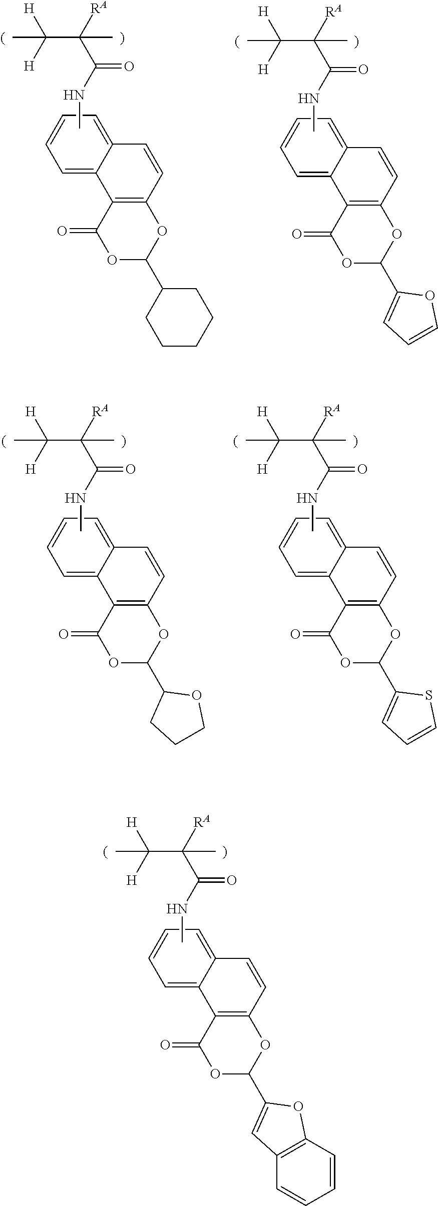

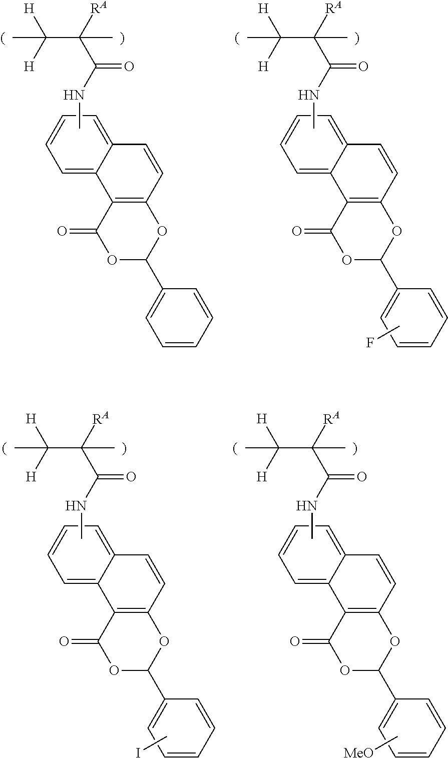

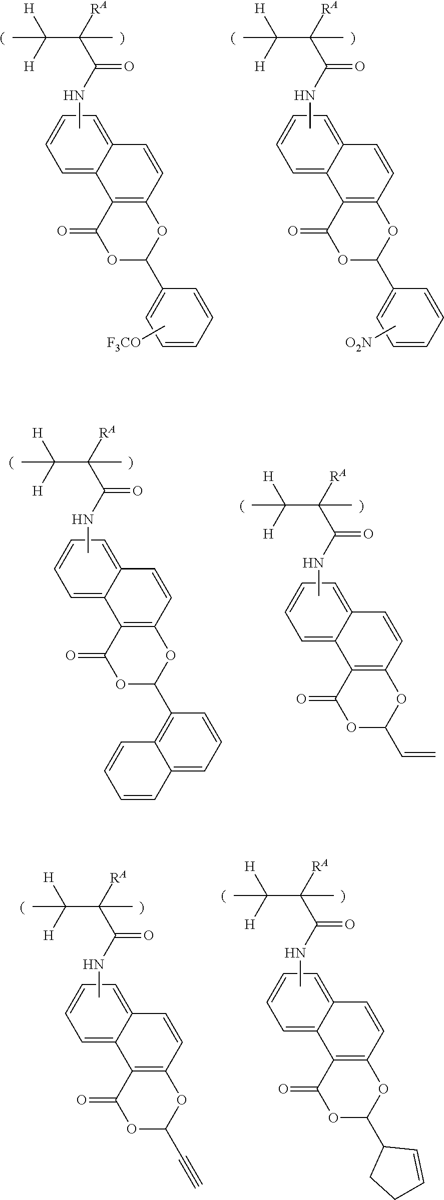

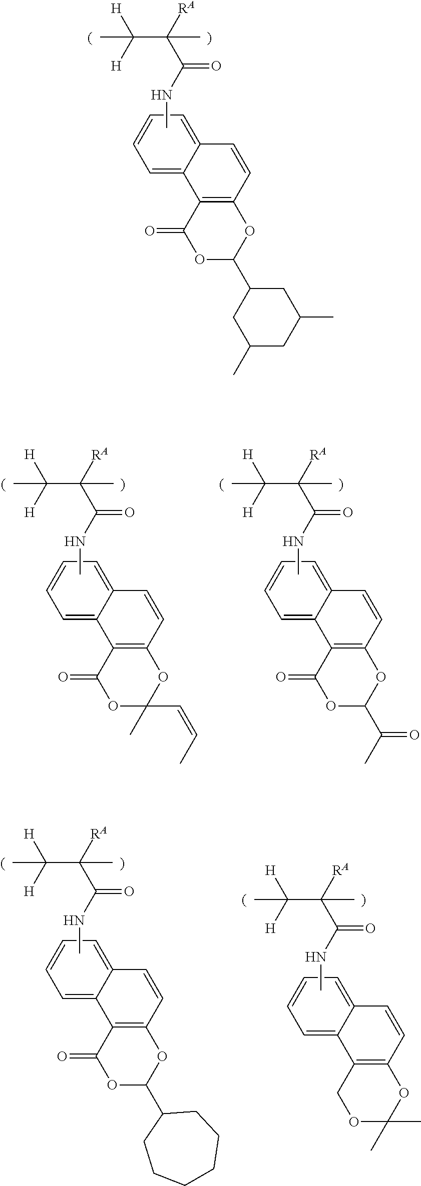

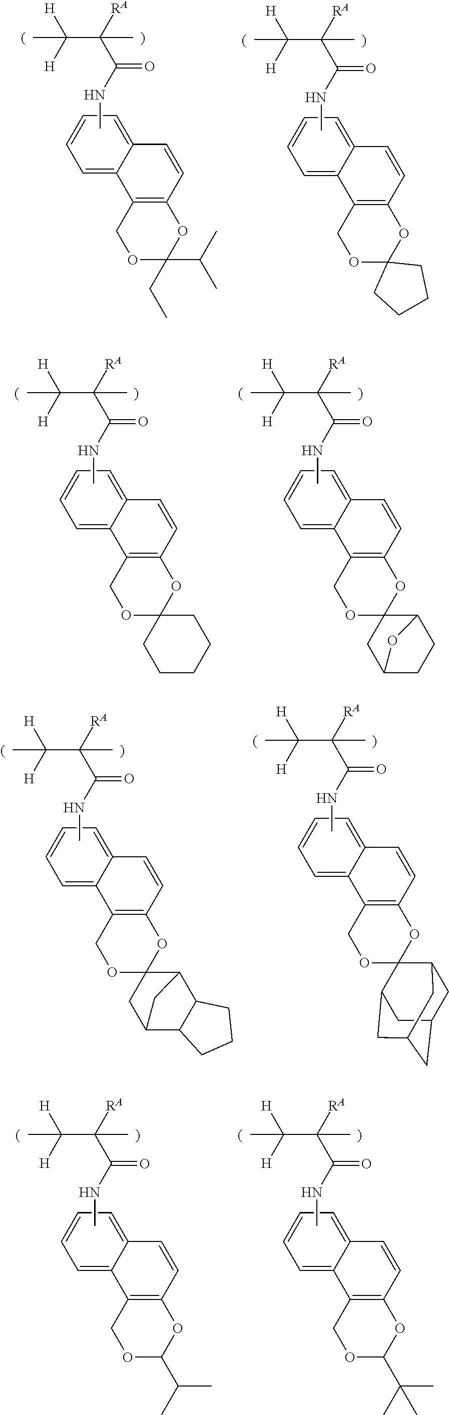

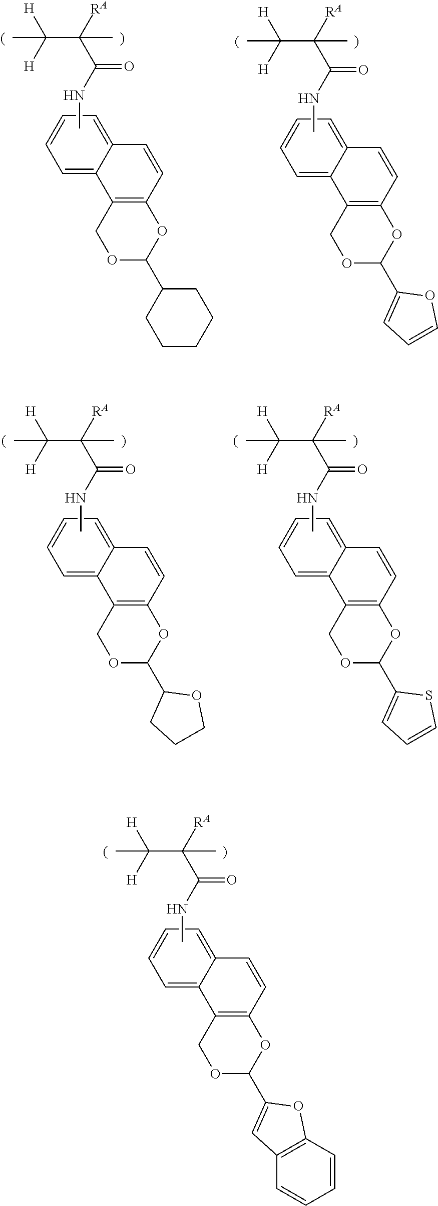

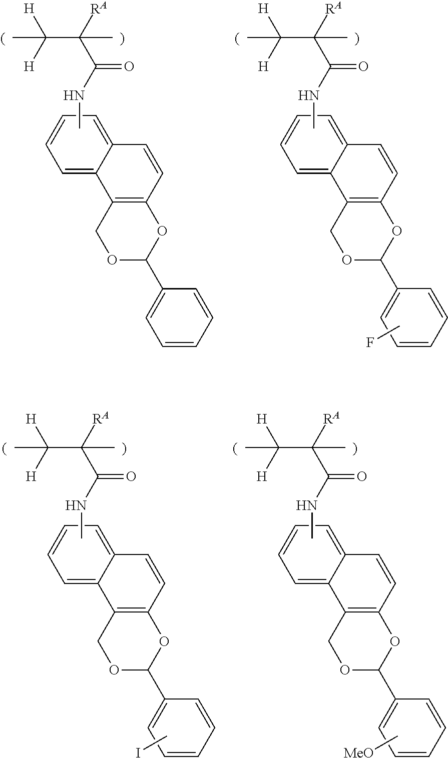

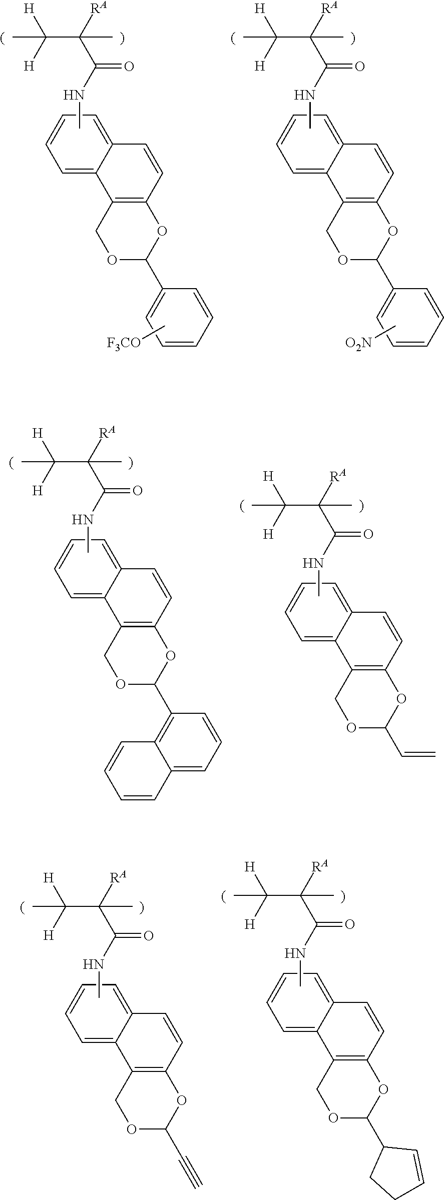

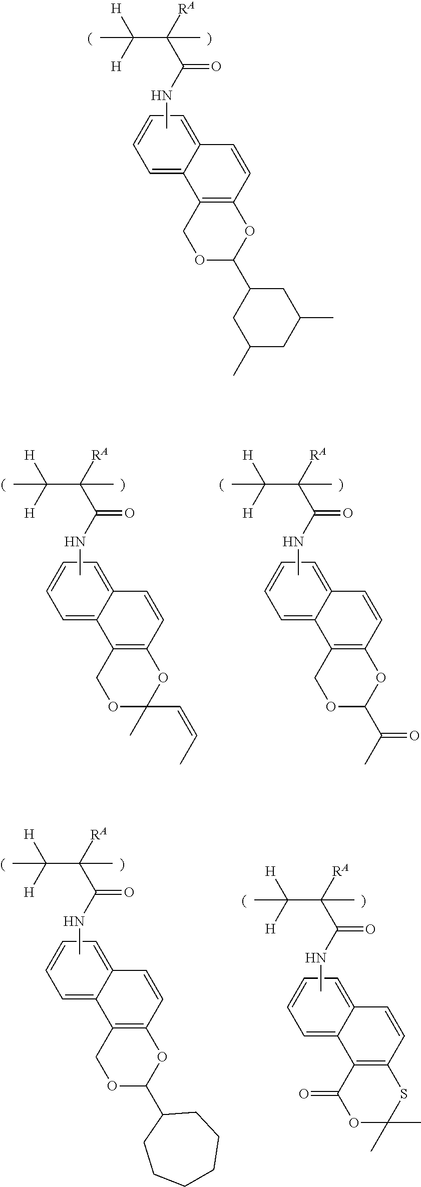

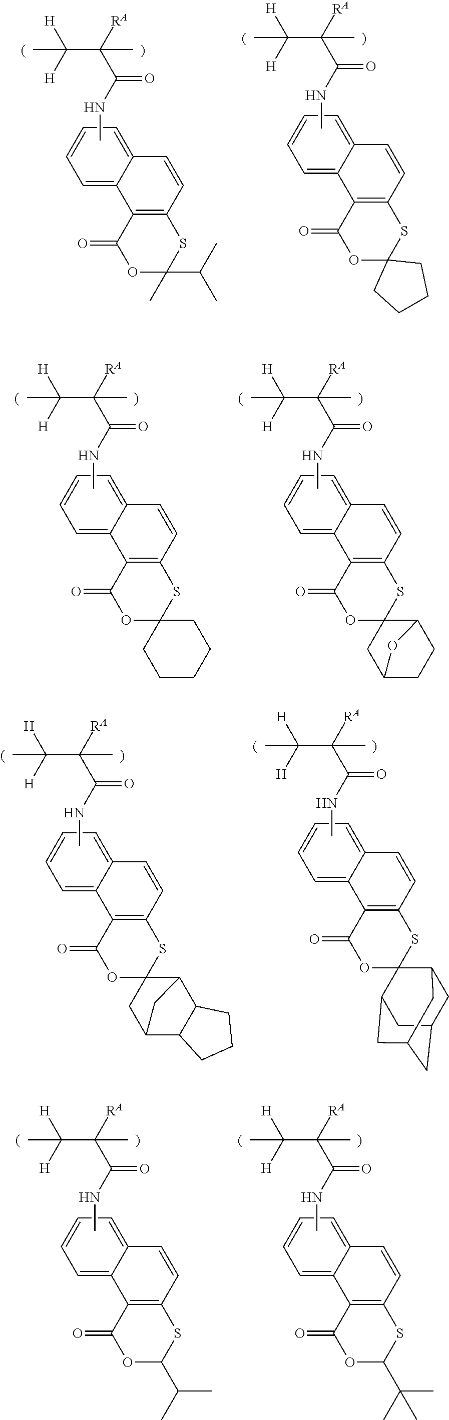

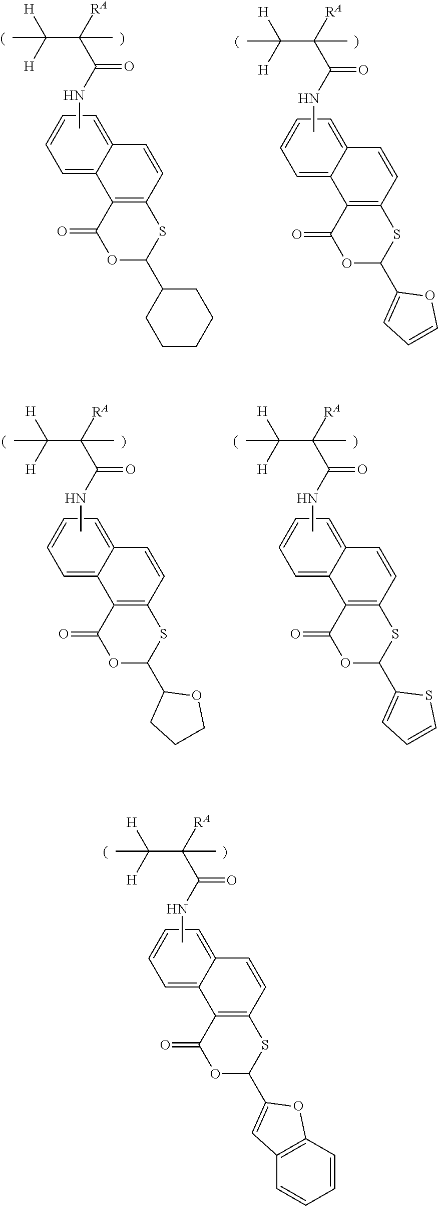

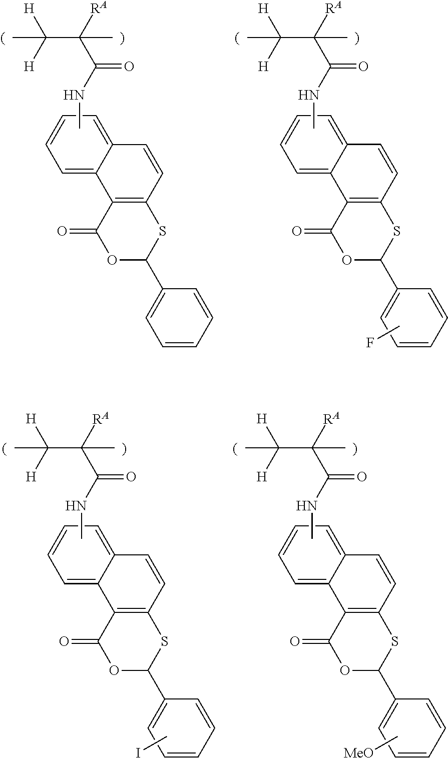

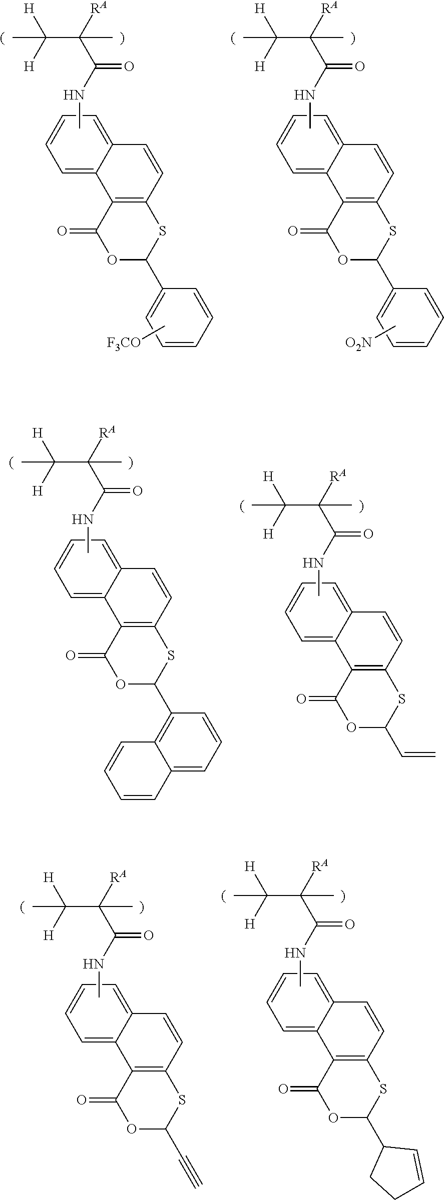

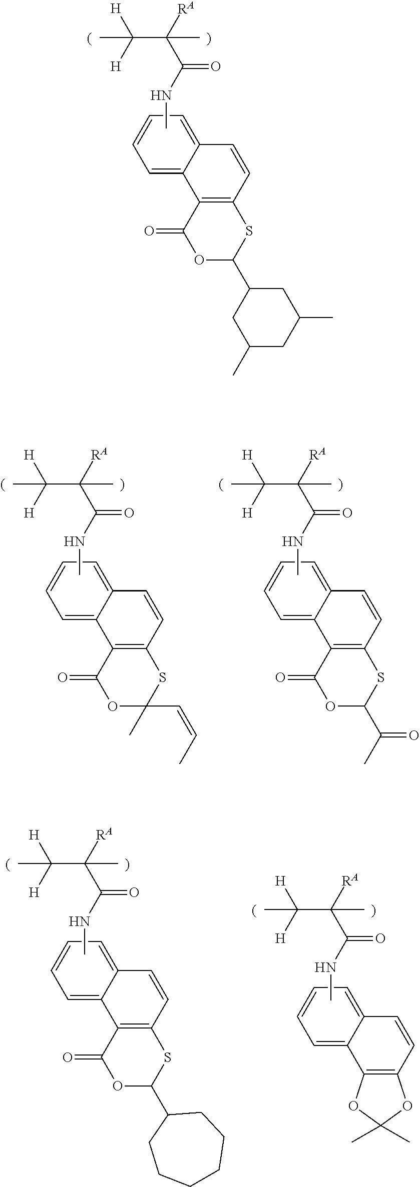

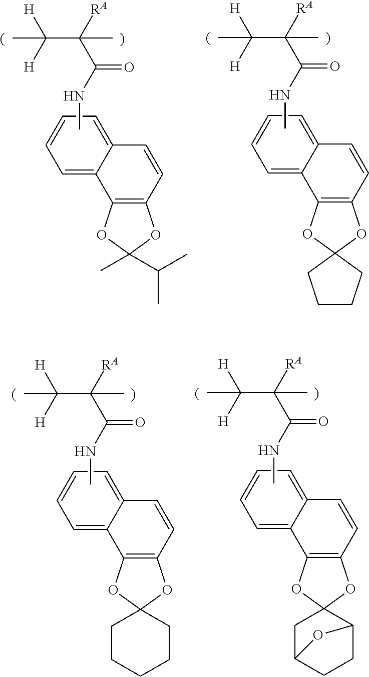

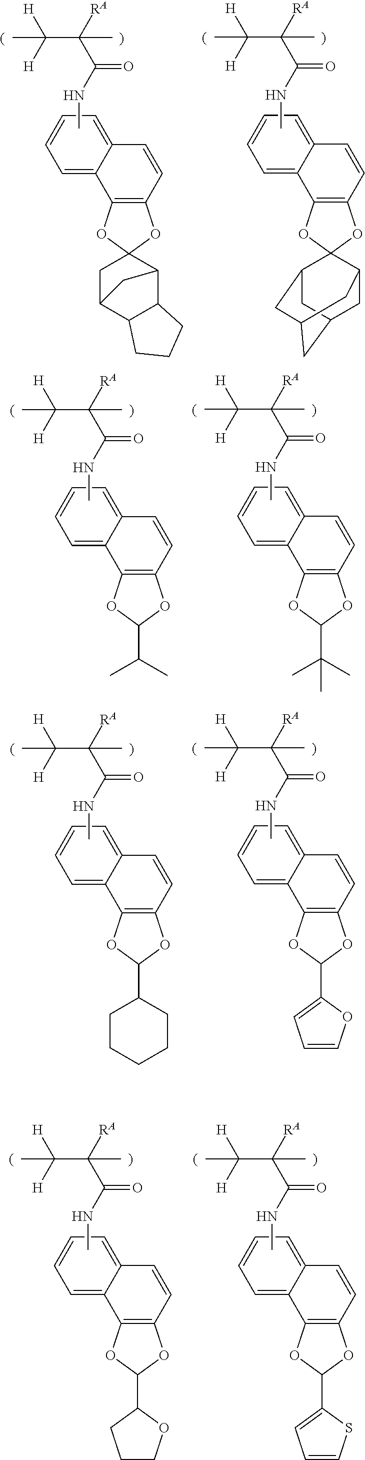

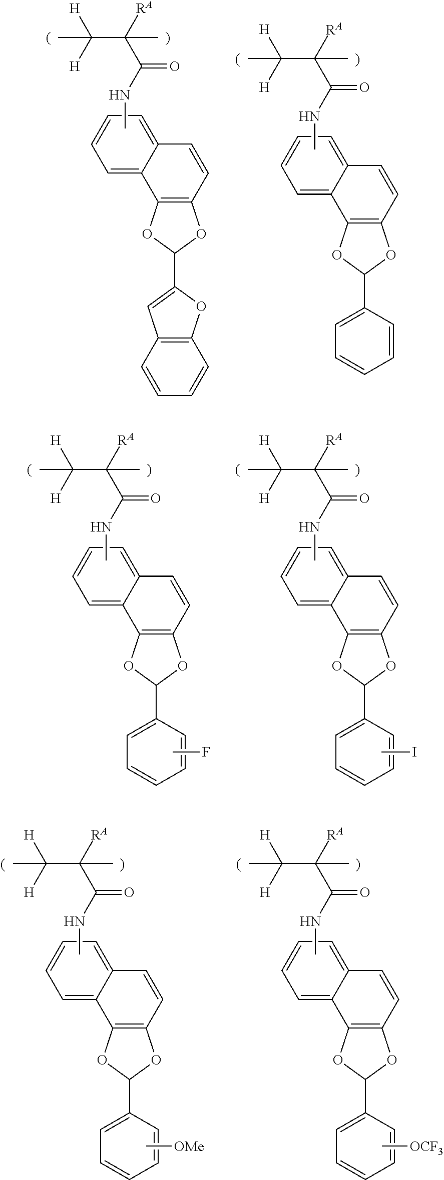

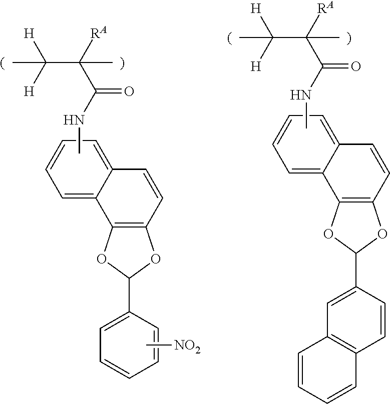

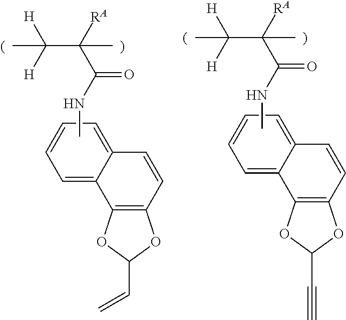

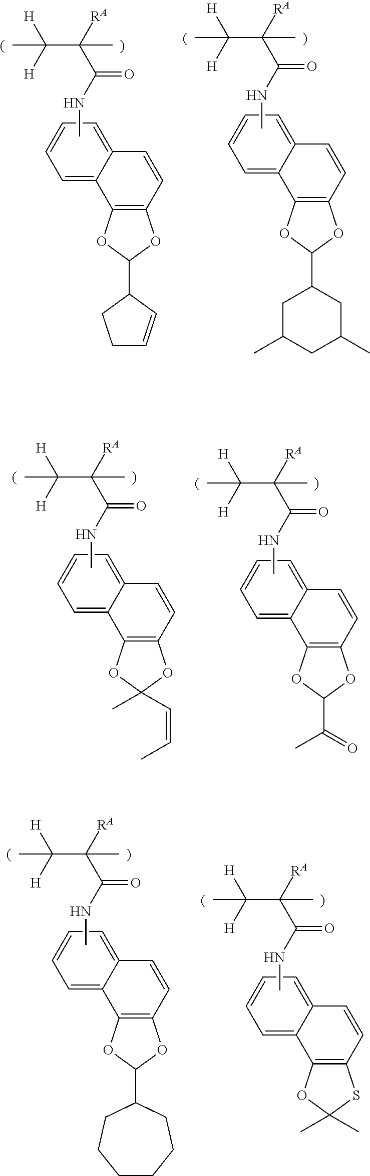

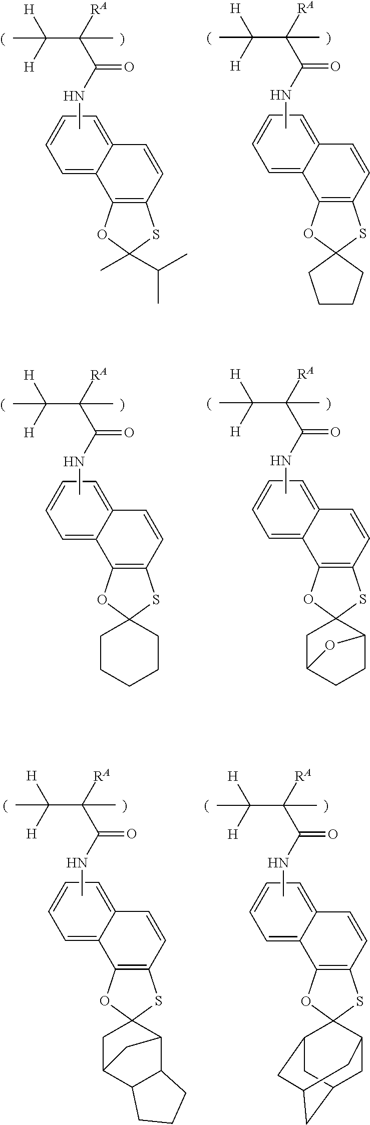

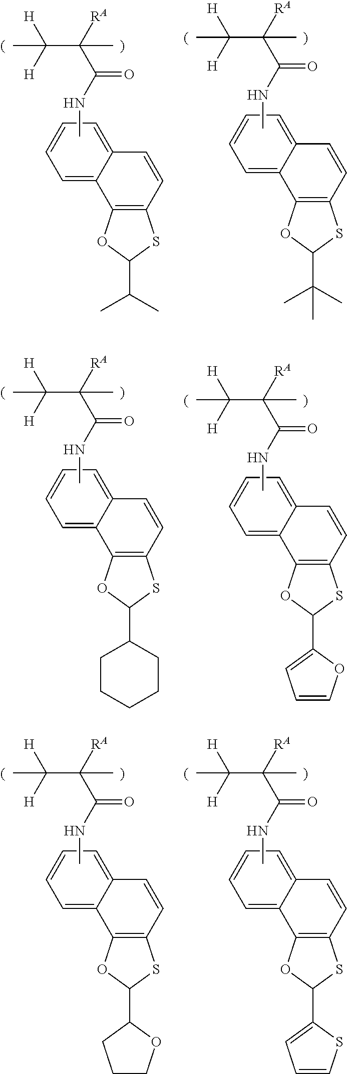

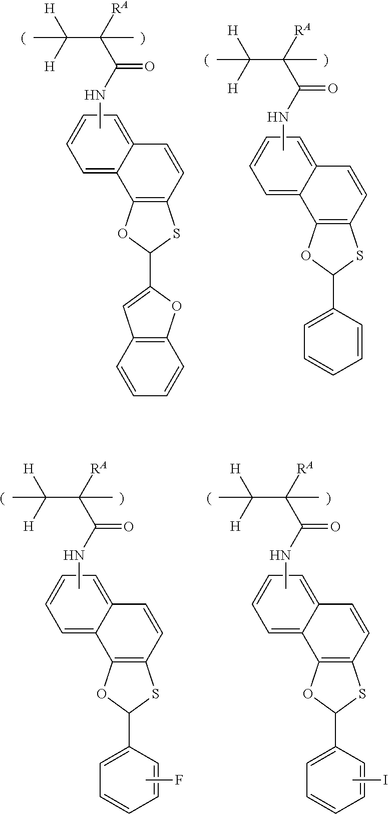

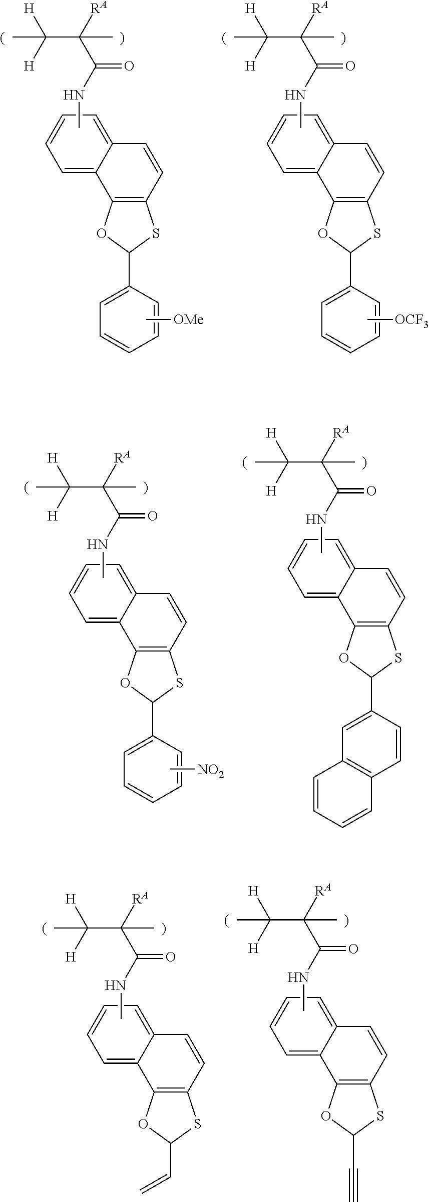

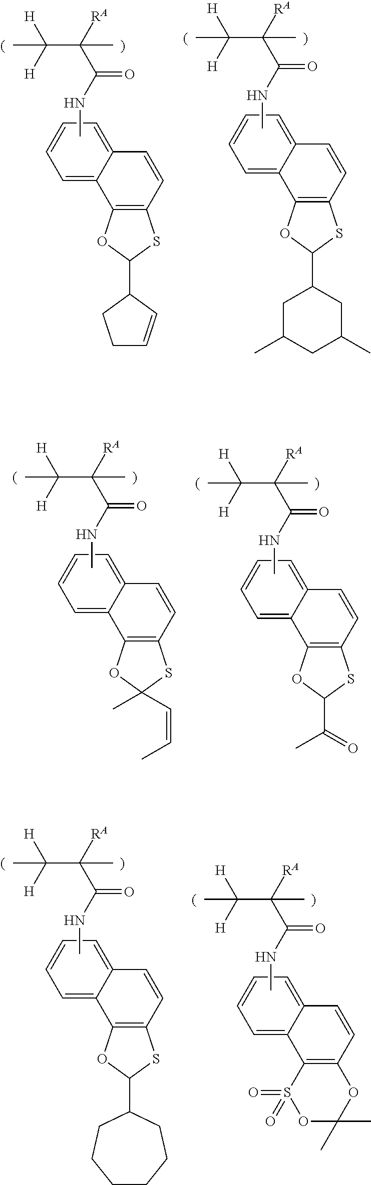

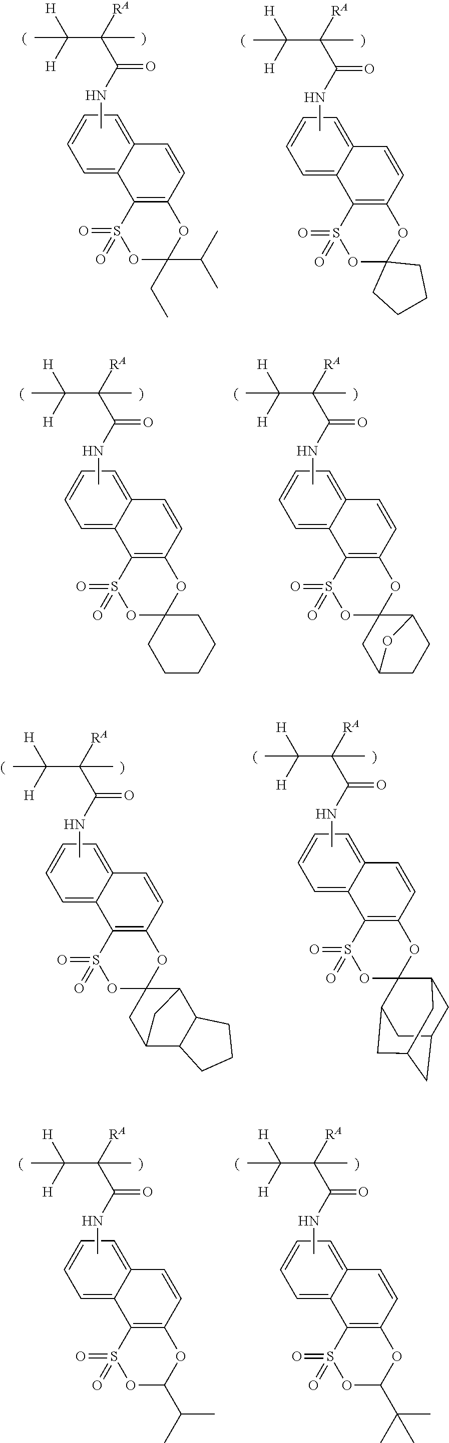

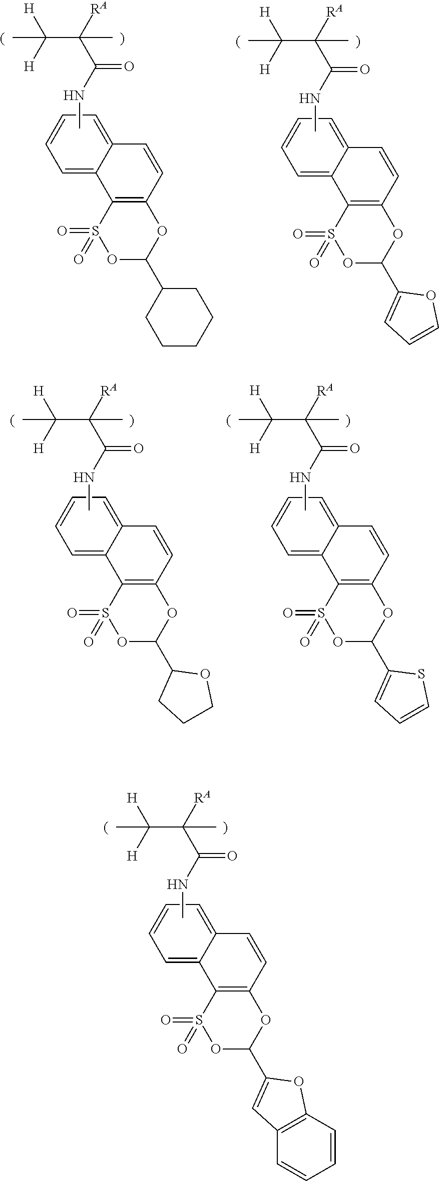

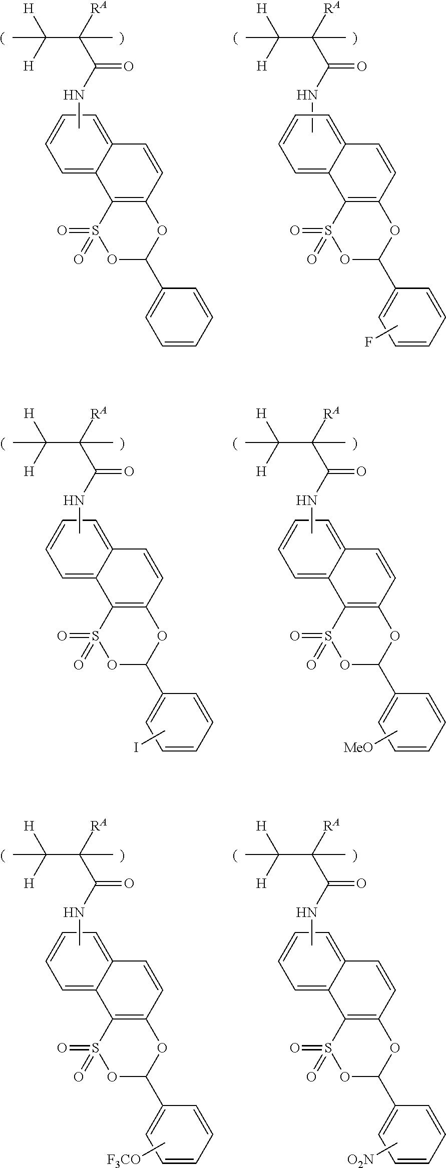

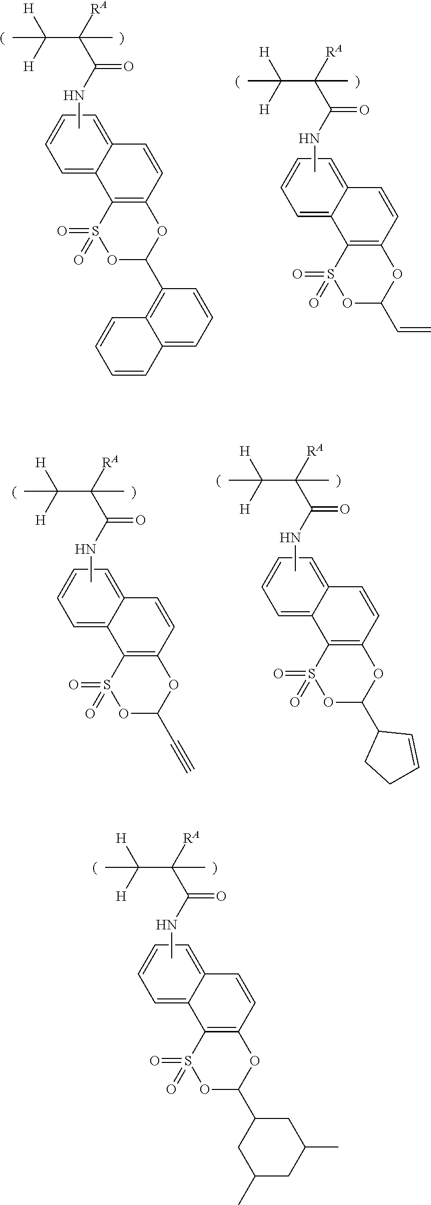

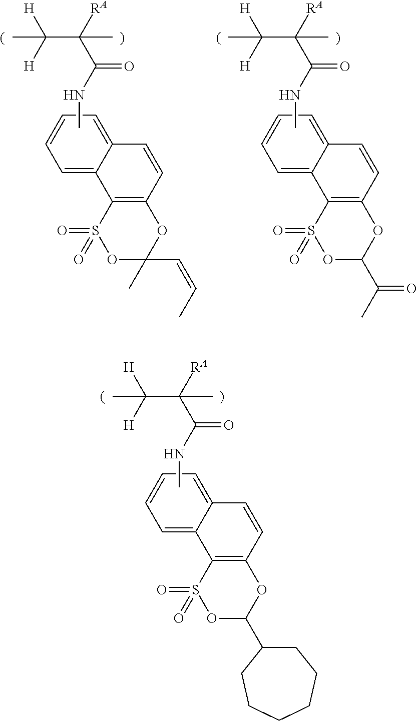

The base polymer may comprise repeat units having the formula (b1) (hereinafter, also referred to as repeat units b1) or repeat units having the formula (b2) (hereinafter, also referred to as repeat units b2).

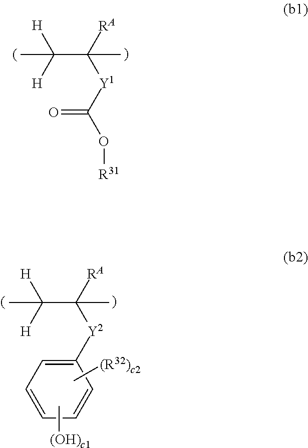

In formulae (b1) and (b2), R^{A} is each independently hydrogen, fluorine, methyl or trifluoromethyl. Y¹ is a single bond, phenylene group, naphthylene group, *—C(═O)—O—Y¹¹— or *—C(═O)—N(H)—Y¹¹—, and the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen. Y¹¹ is a C₁-C₁₀ saturated hydrocarbylene group, phenylene group or naphthylene group, the saturated hydrocarbylene group may contain hydroxy, ether bond, ester bond or lactone ring. Y² is a single bond or *—C(═O)—O—. * designates a point of attachment to the carbon atom in the backbone. R³¹ is hydrogen or a C₁-C₂₀ group which contains at least one structure selected from hydroxy exclusive of phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—). R³² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom. When c2 is 2, 3 or 4, a plurality of R³² may be identical or different. c1 is 1, 2, 3 or 4. c2 is 0, 1, 2, 3 or 4. Provided that 1≤c1+c2≤5.

Examples of repeat unit b 1 are shown below, but are not limited thereto. Herein, R^{A} is as defined above.

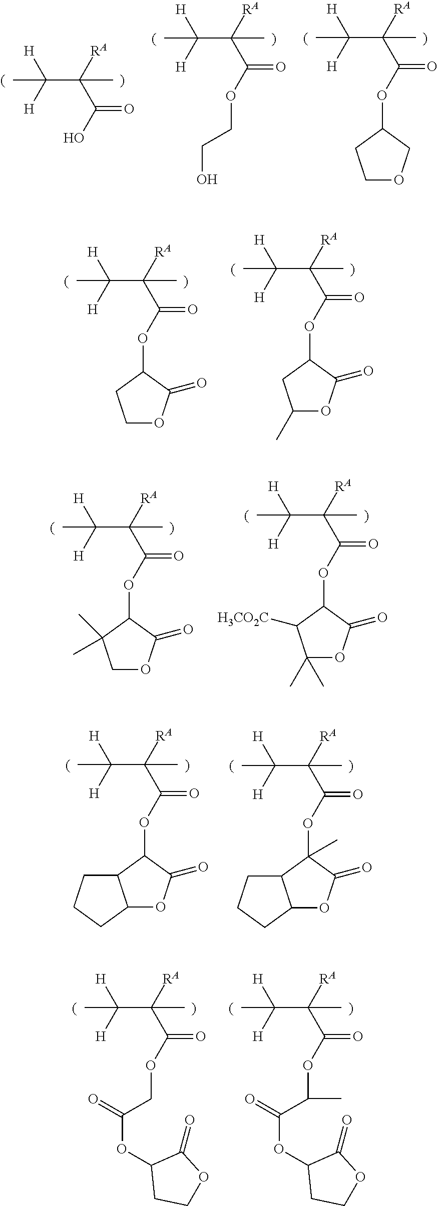

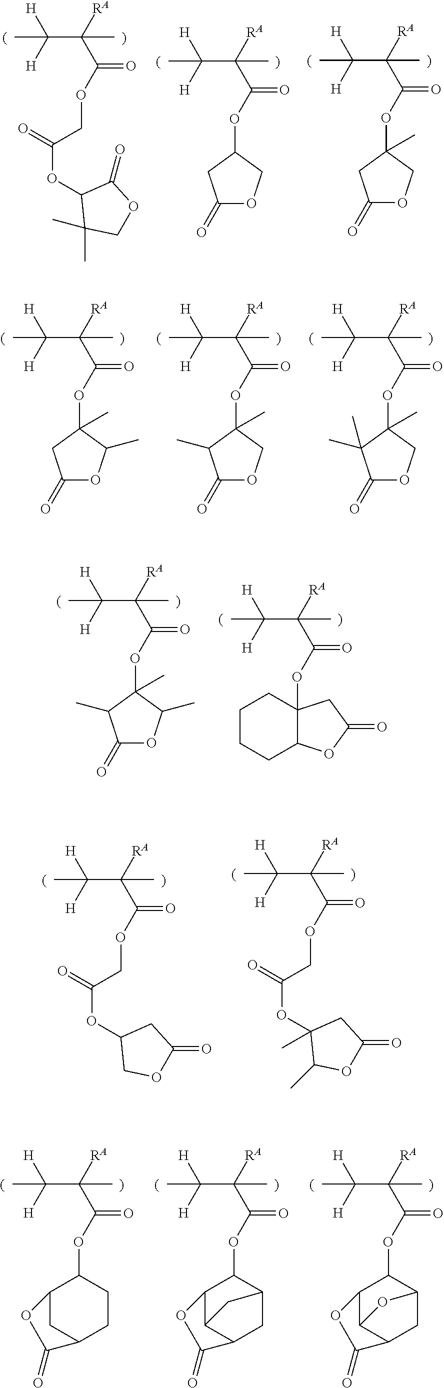

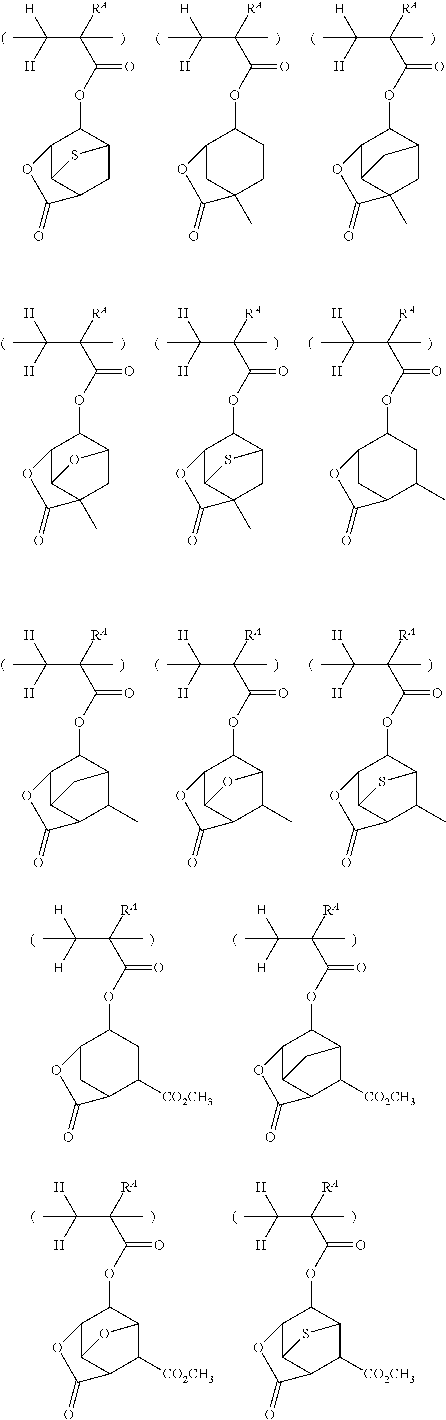

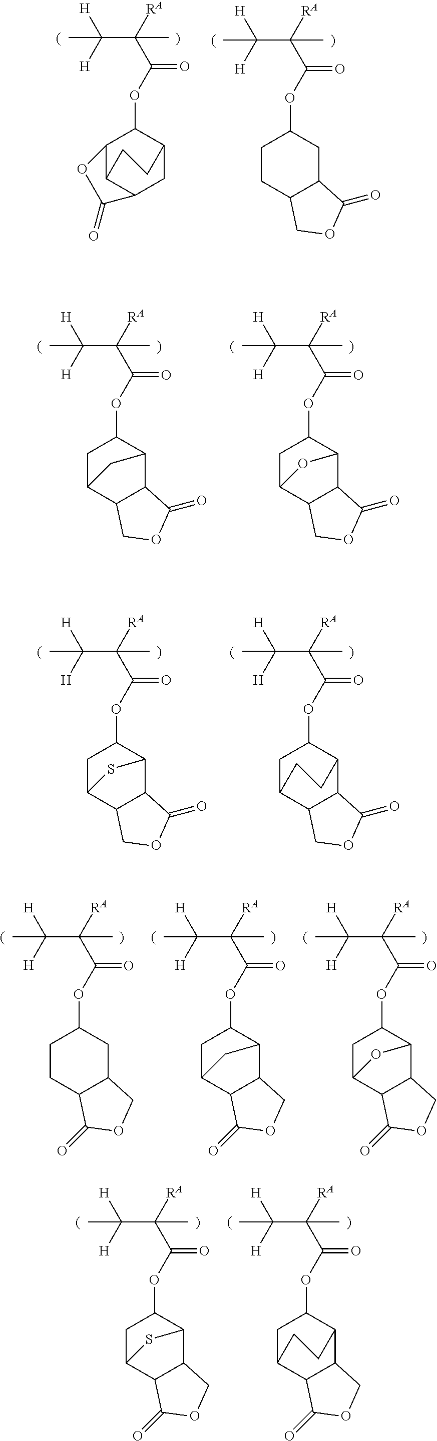

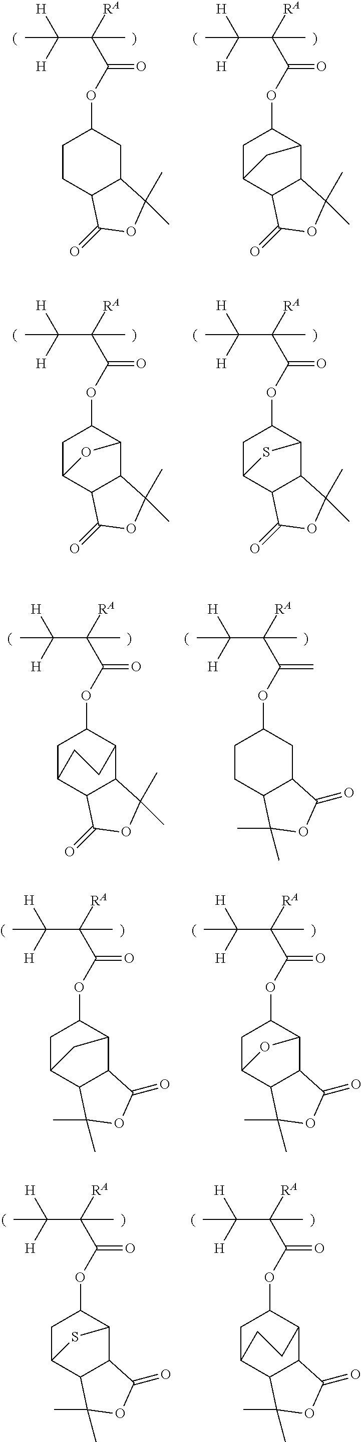

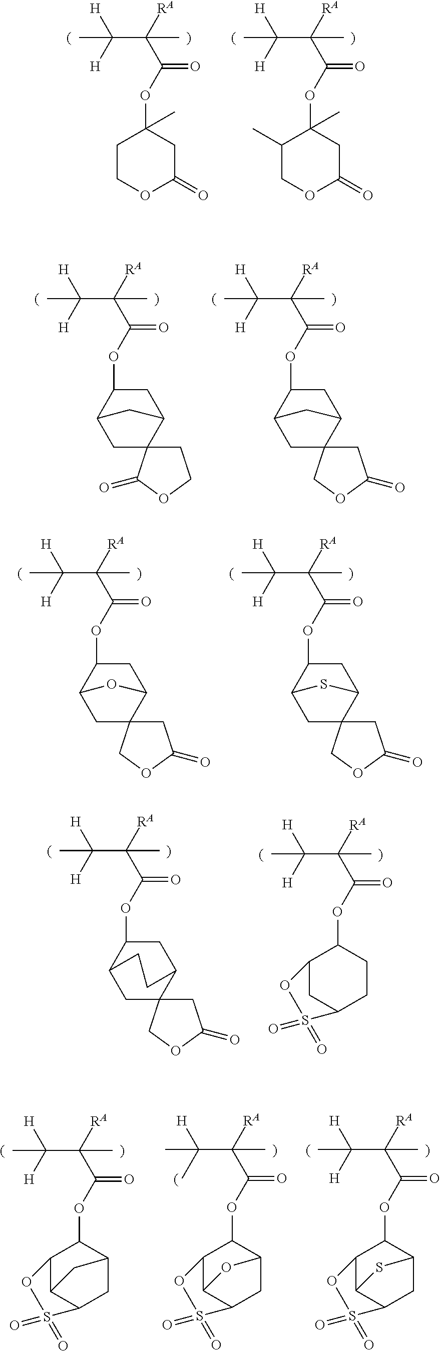

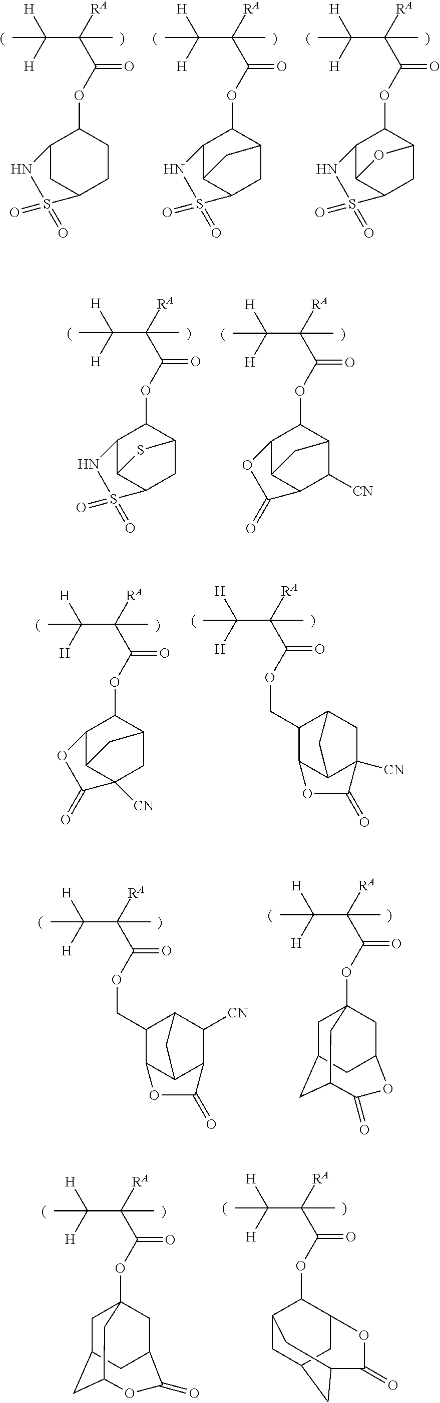

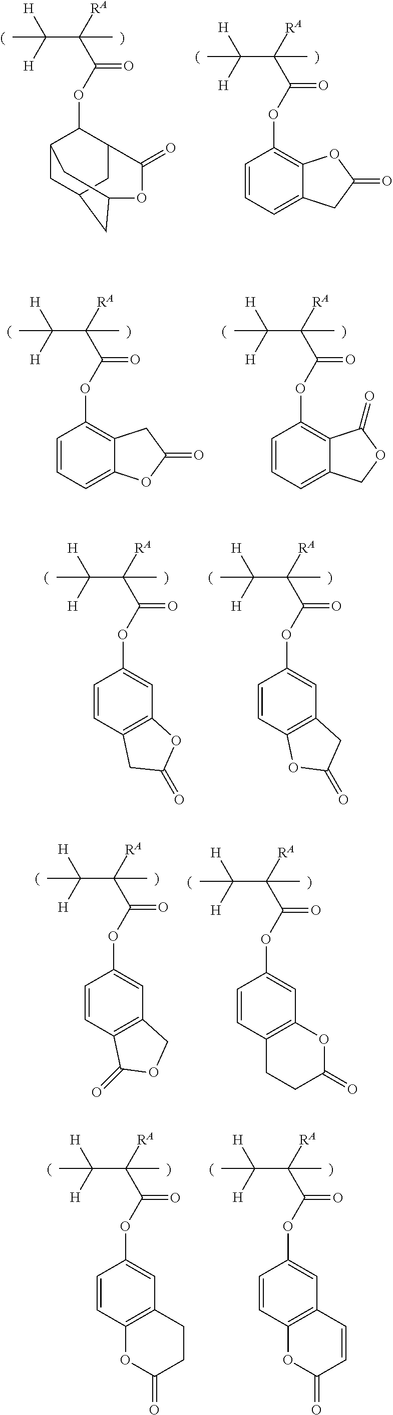

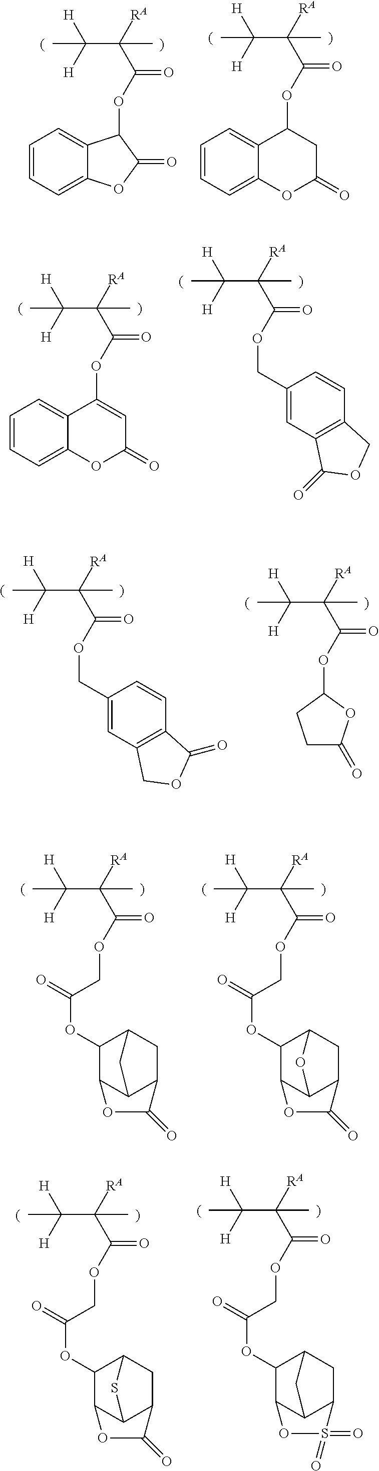

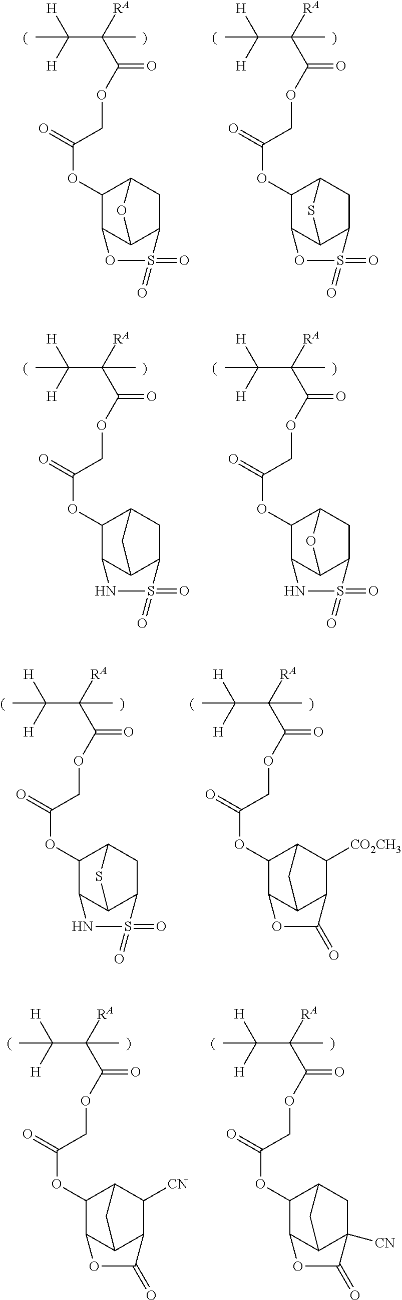

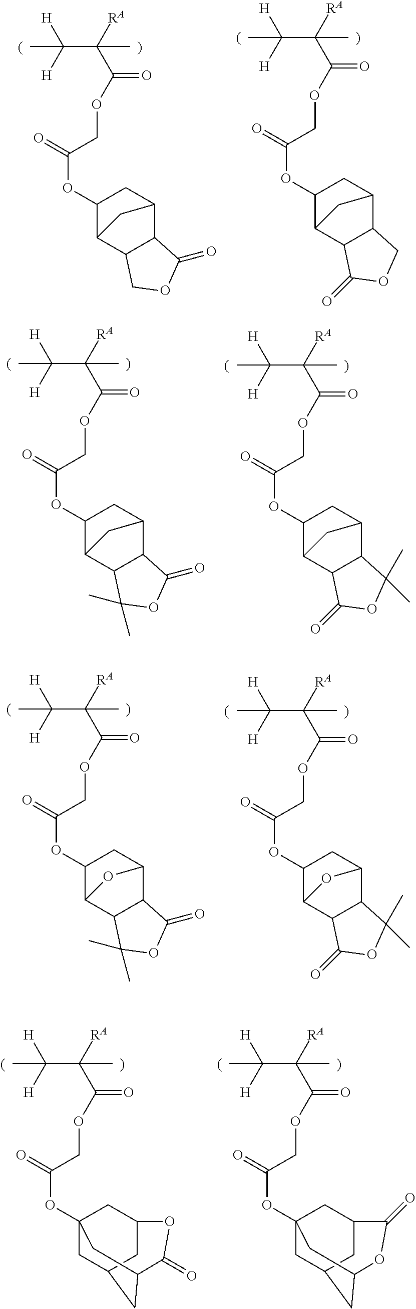

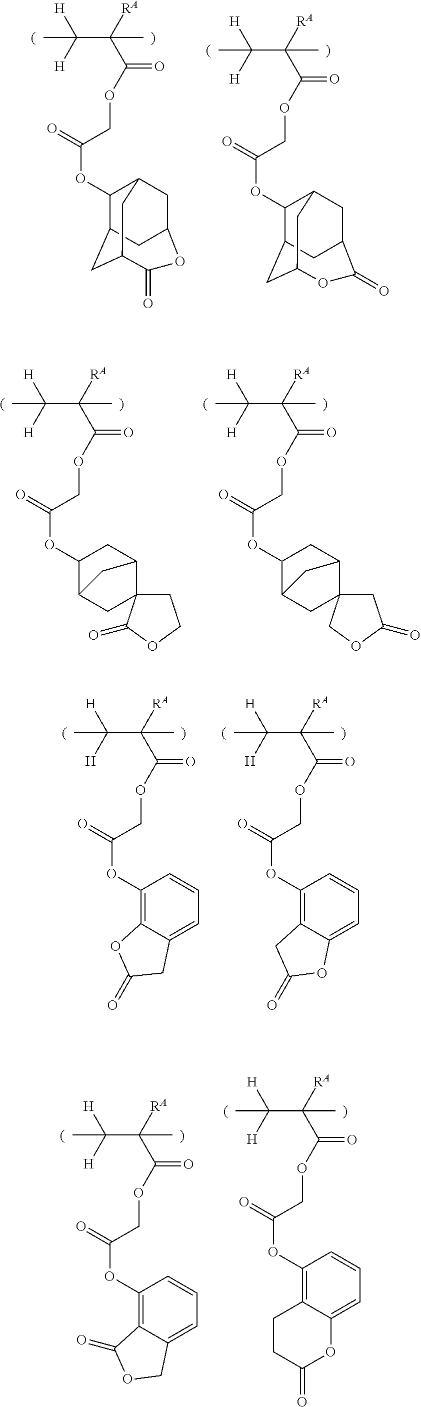

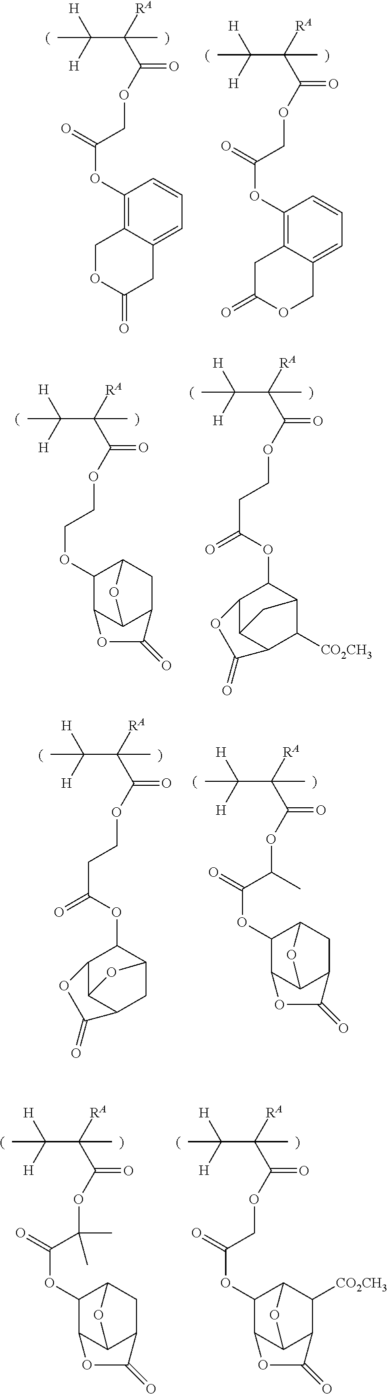

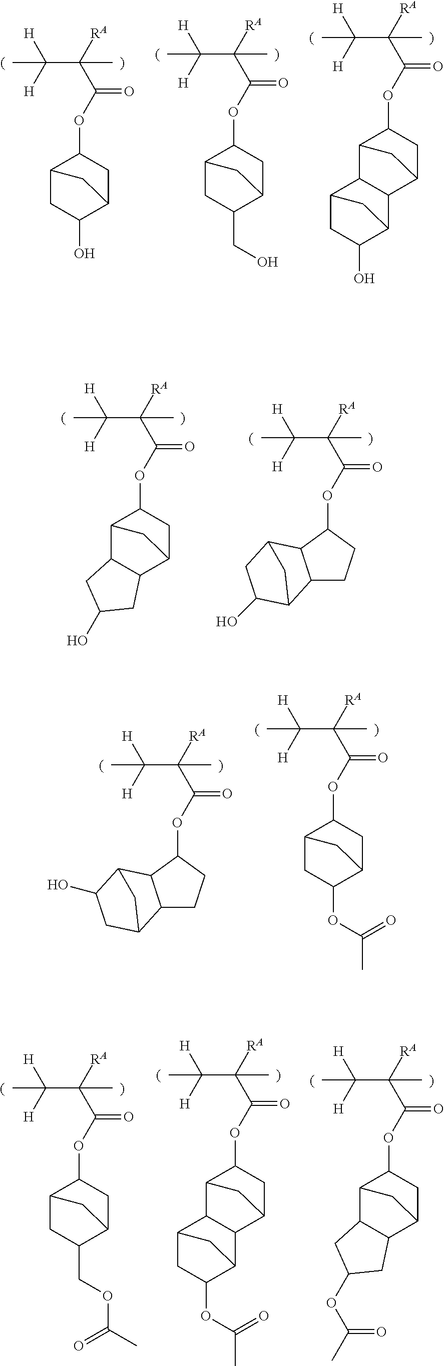

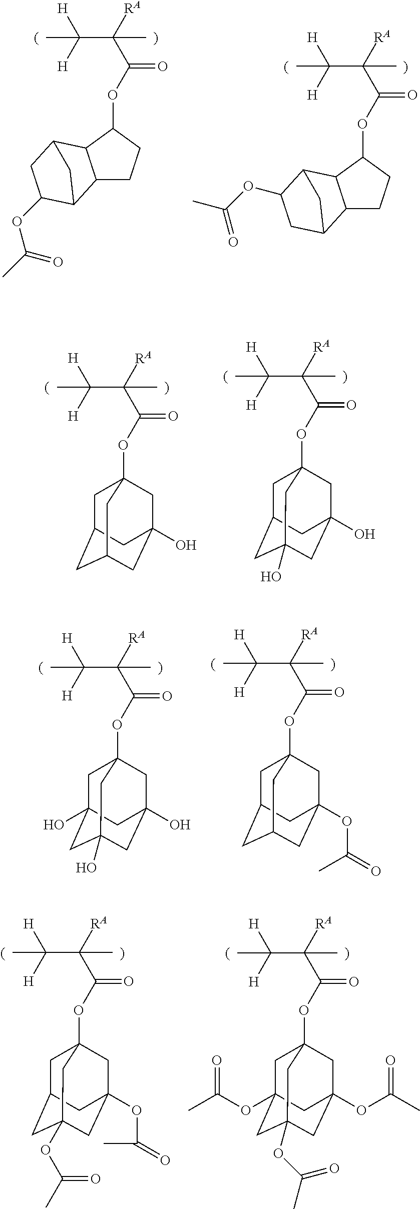

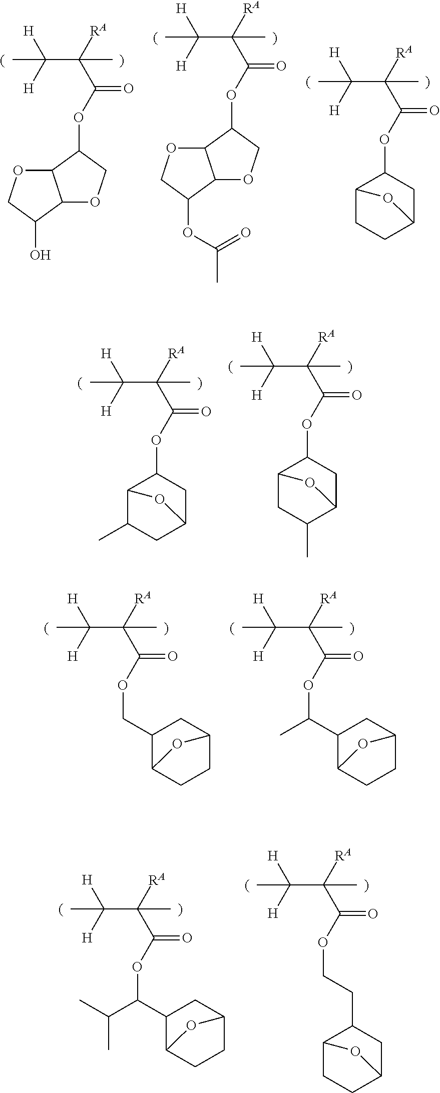

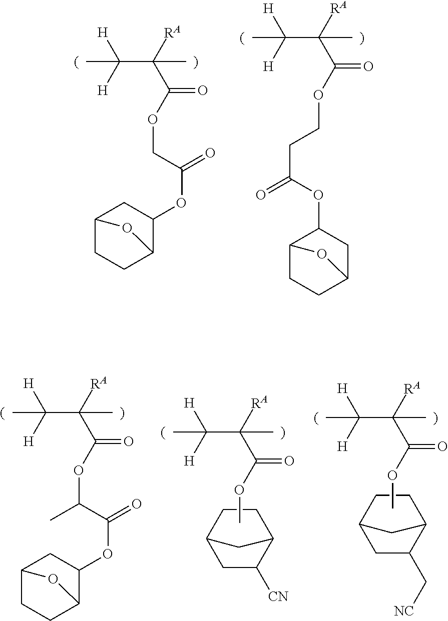

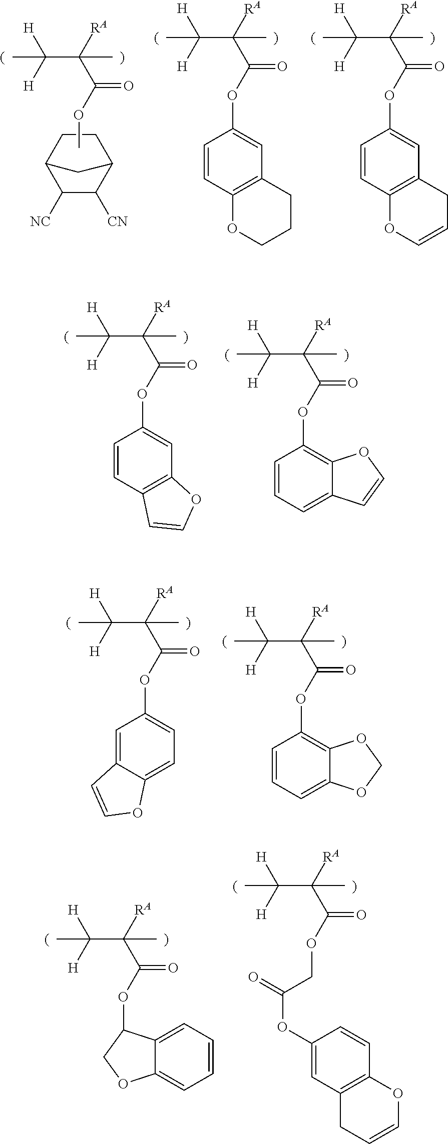

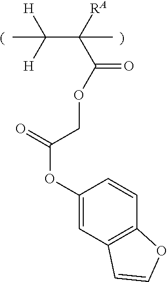

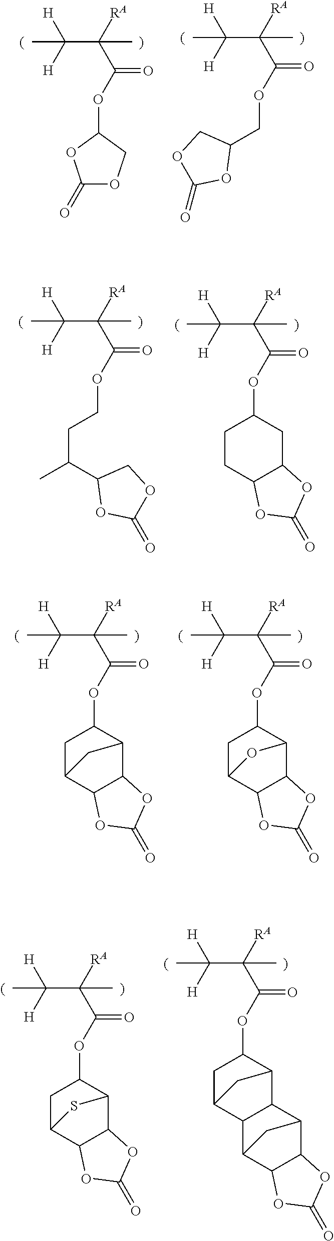

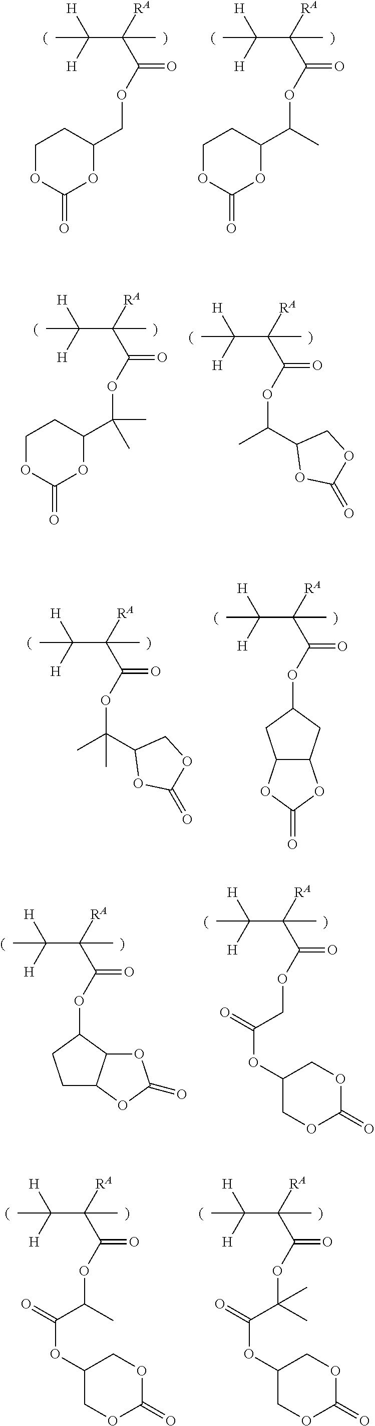

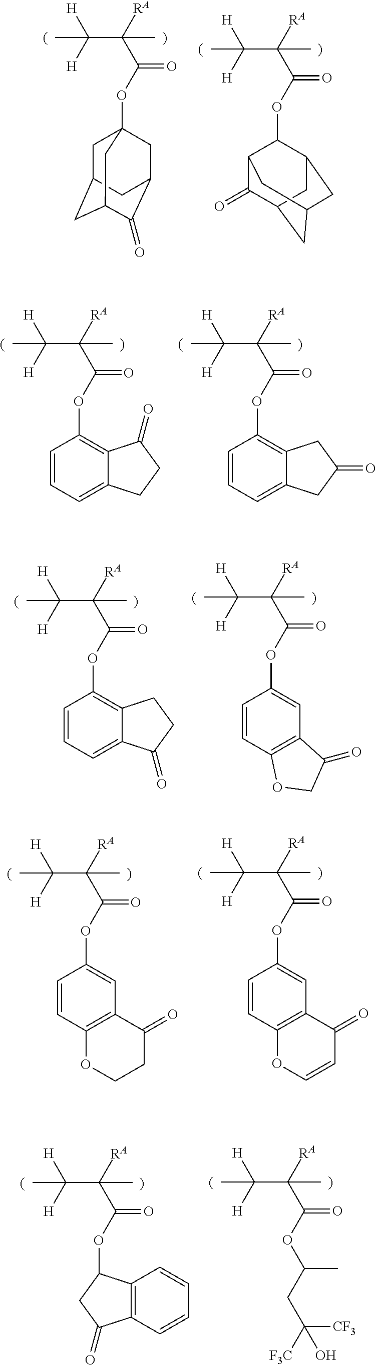

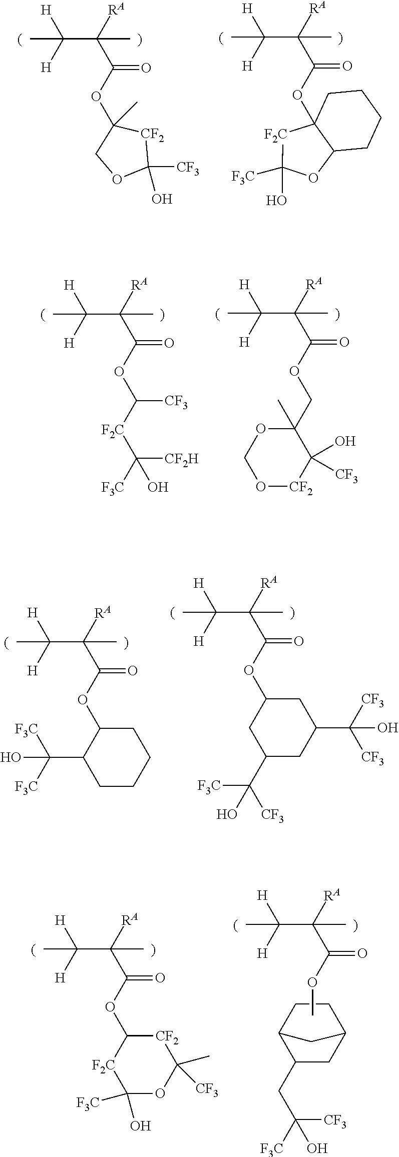

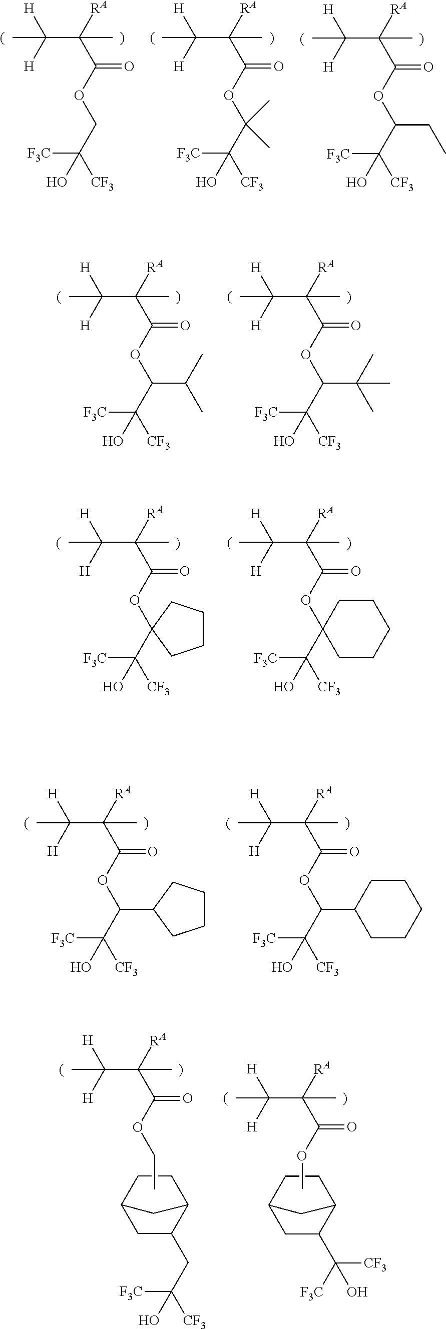

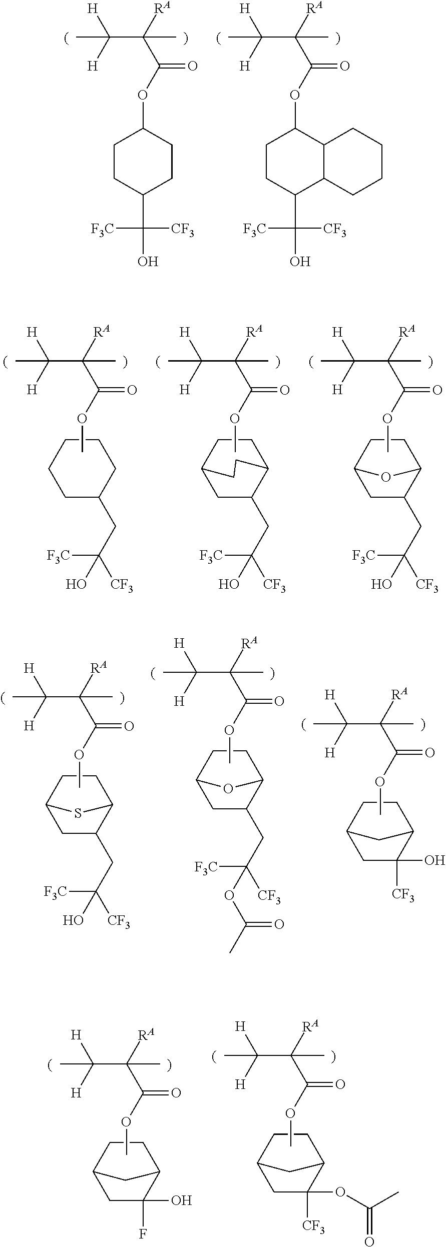

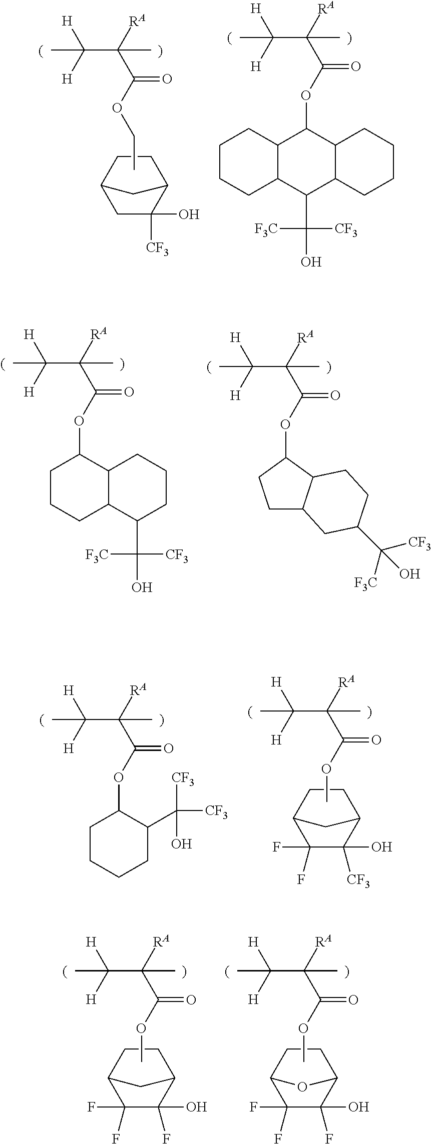

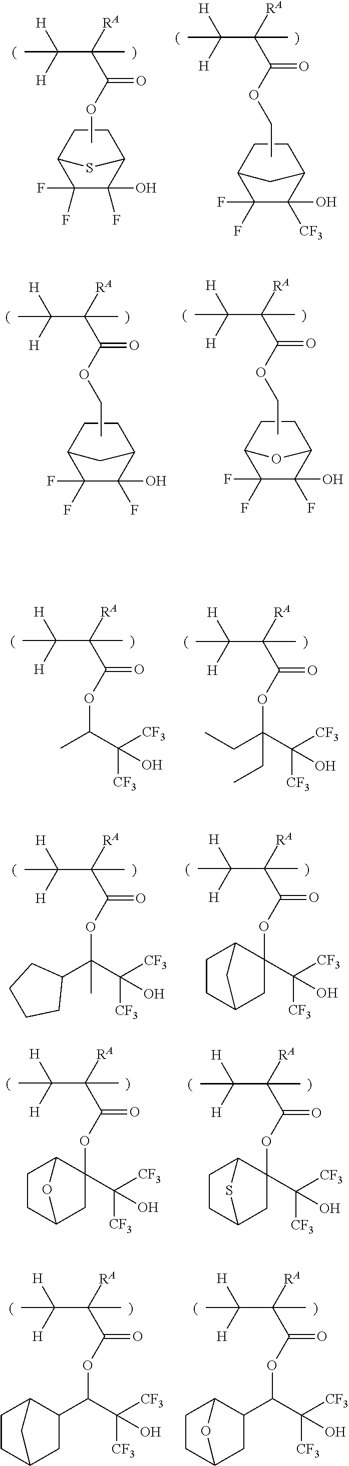

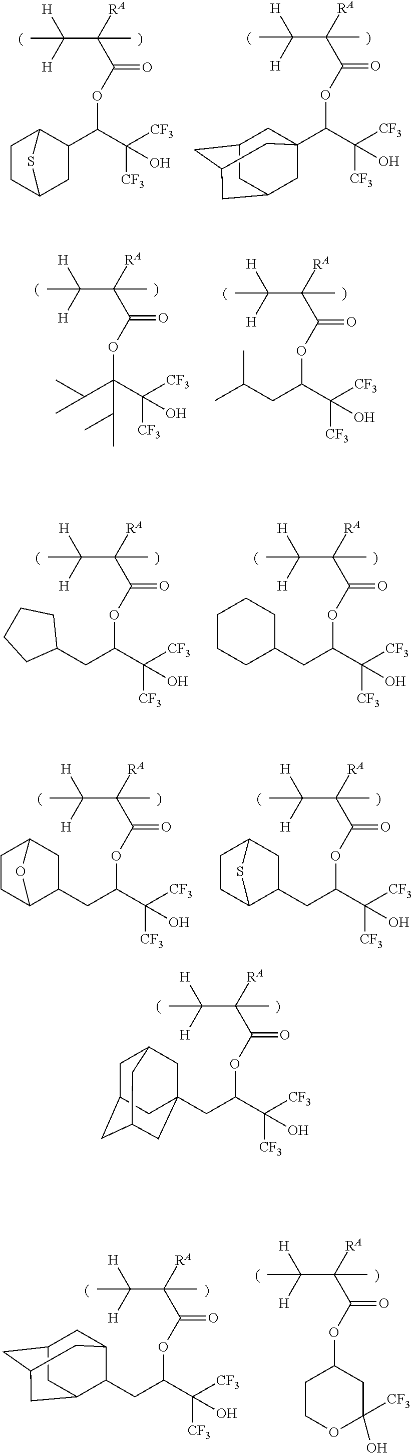

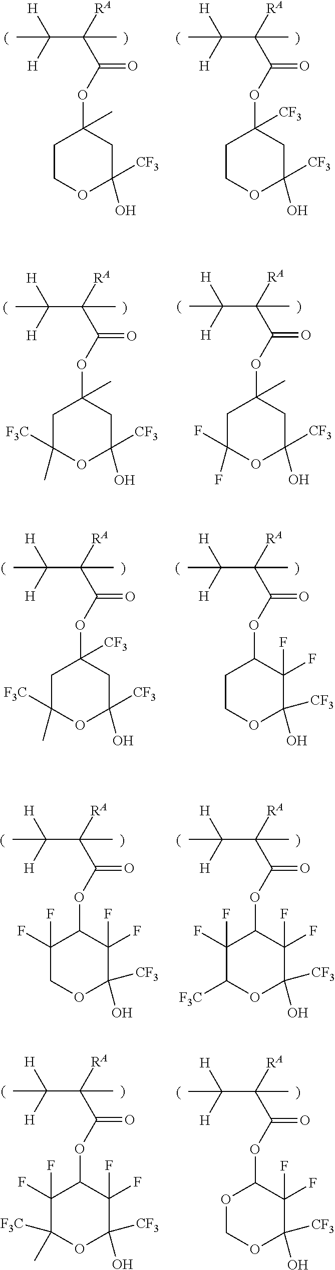

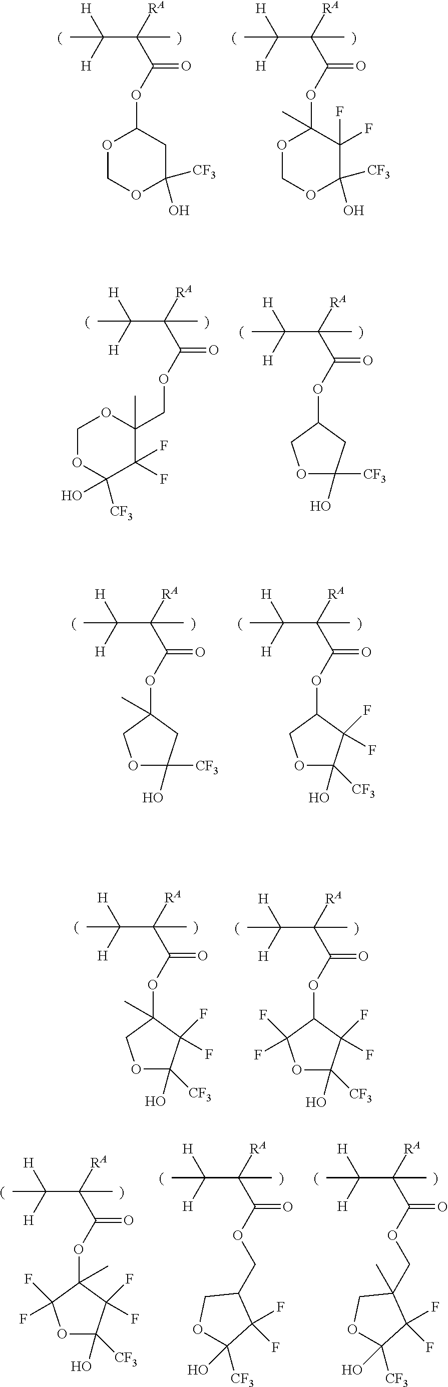

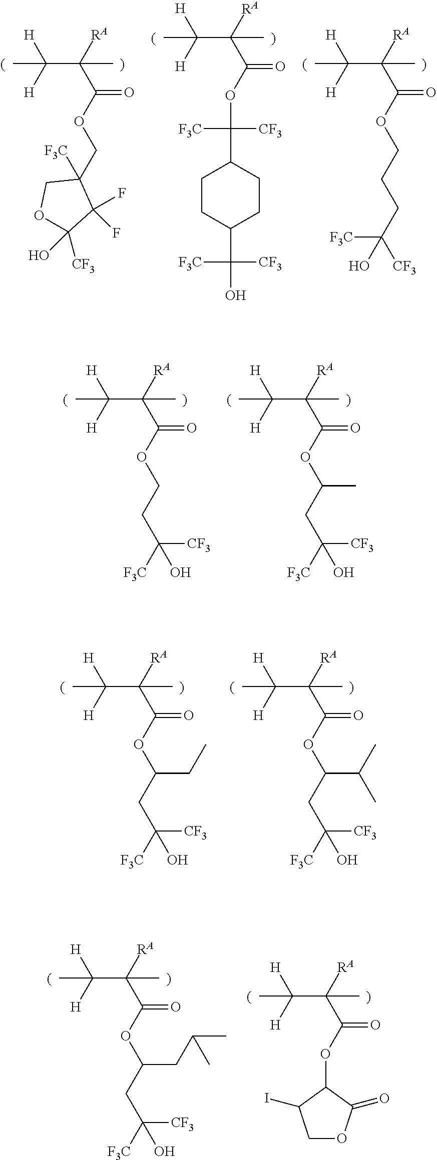

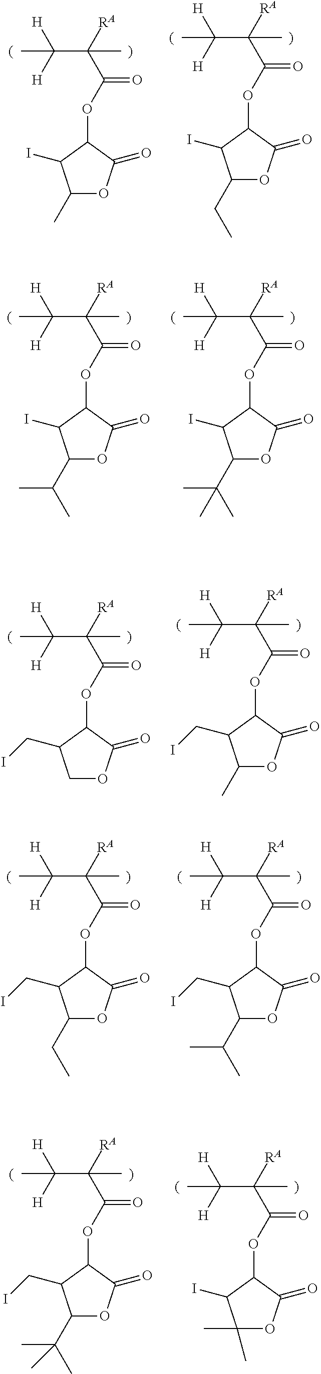

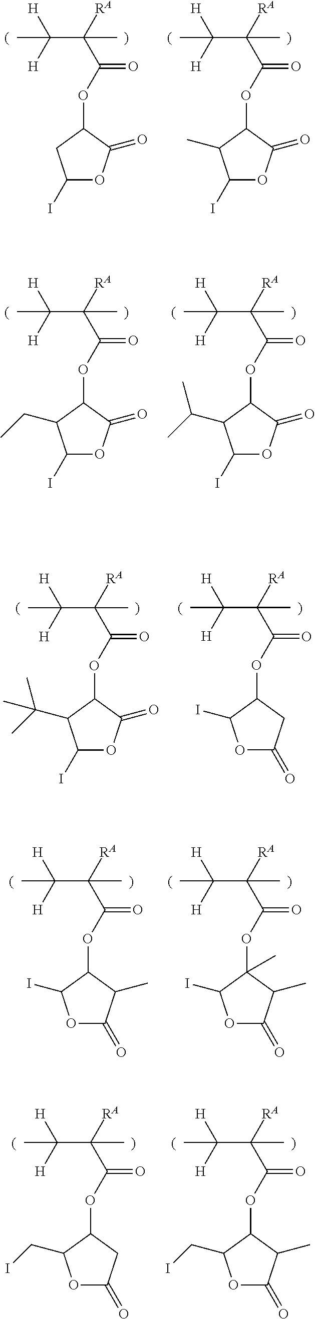

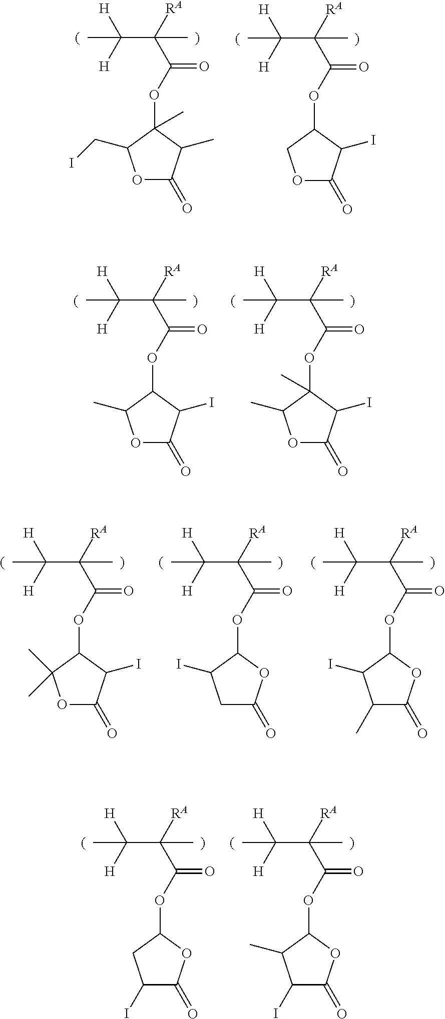

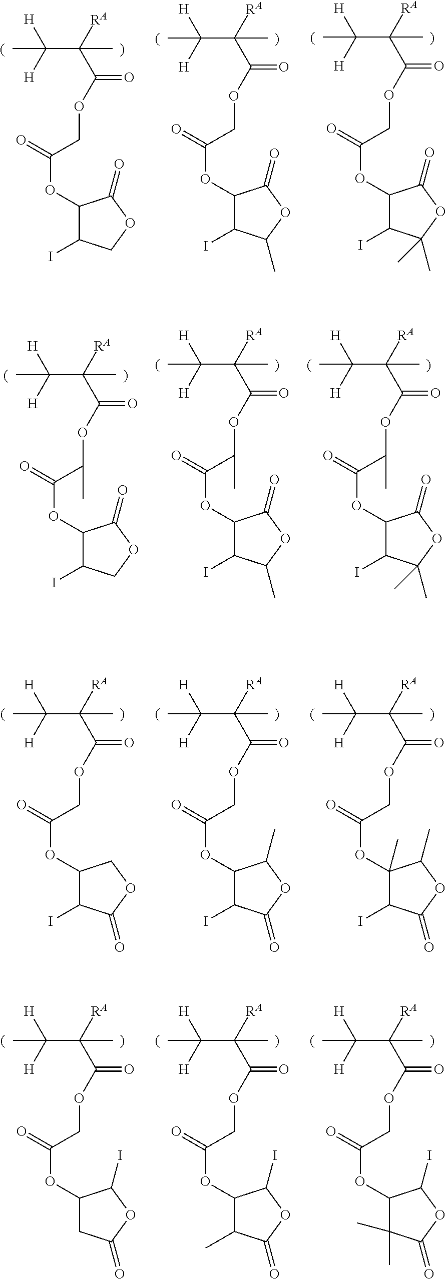

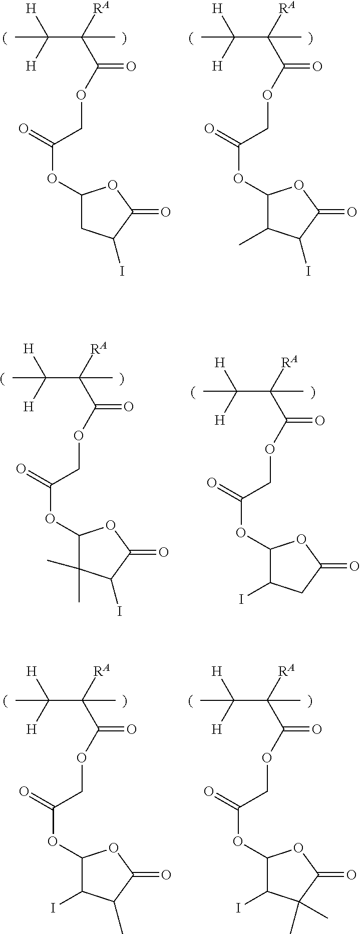

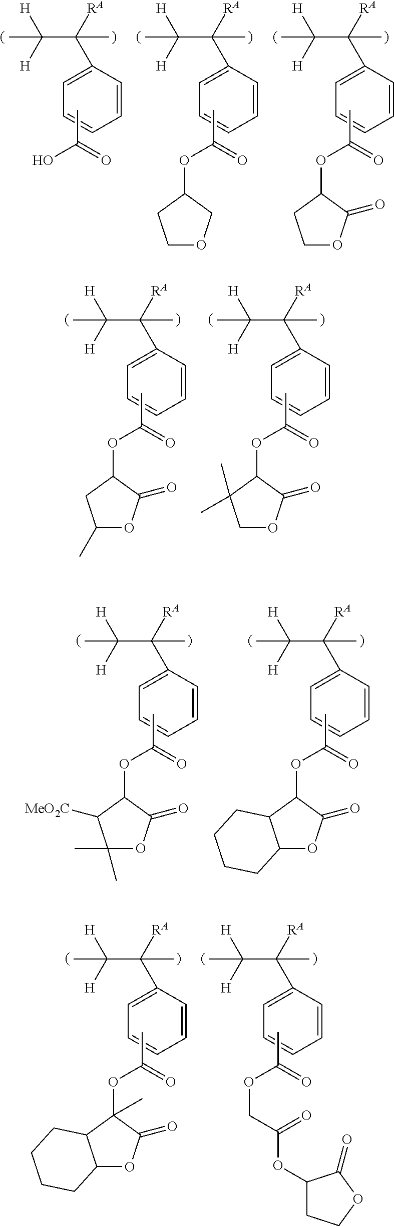

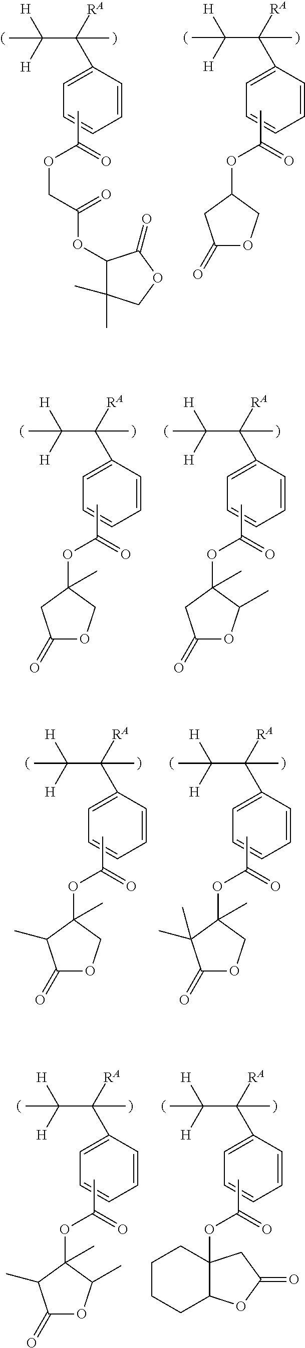

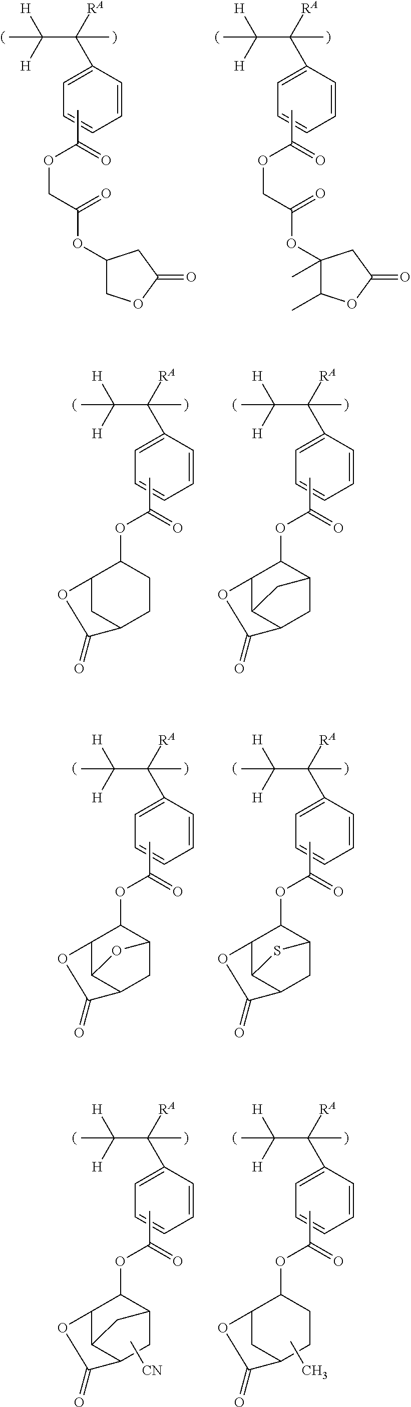

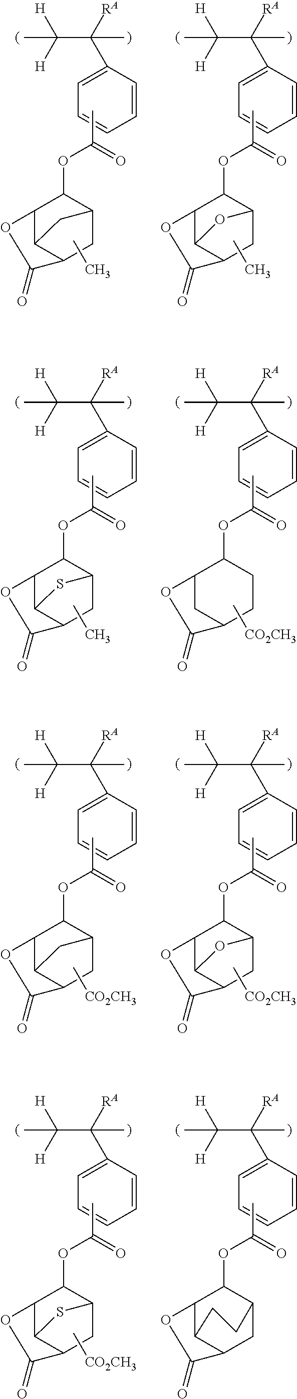

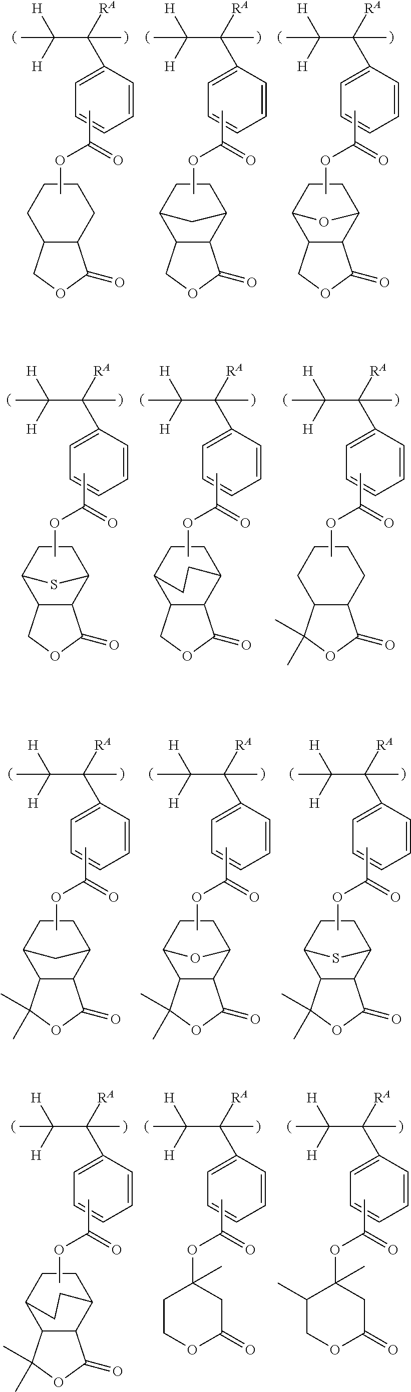

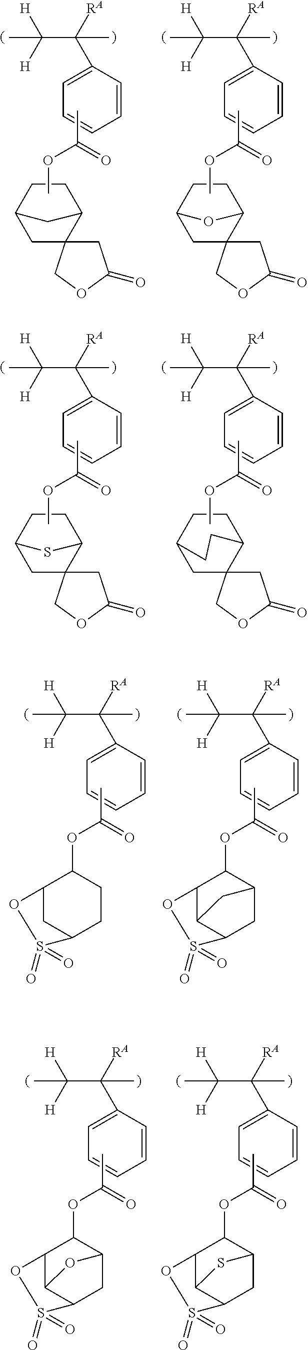

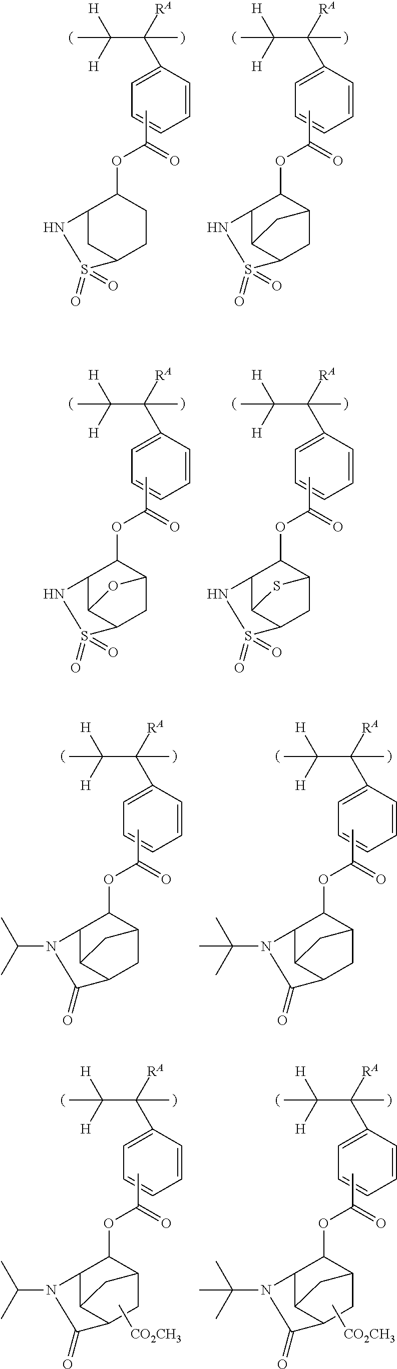

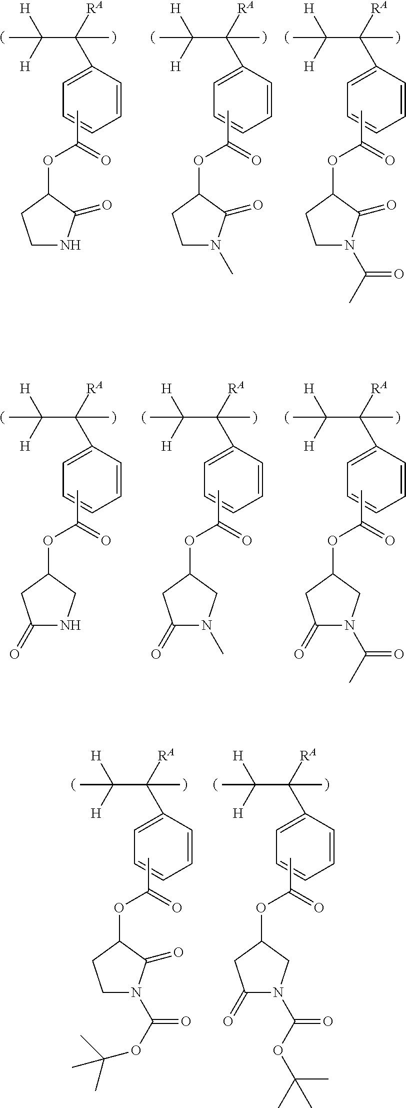

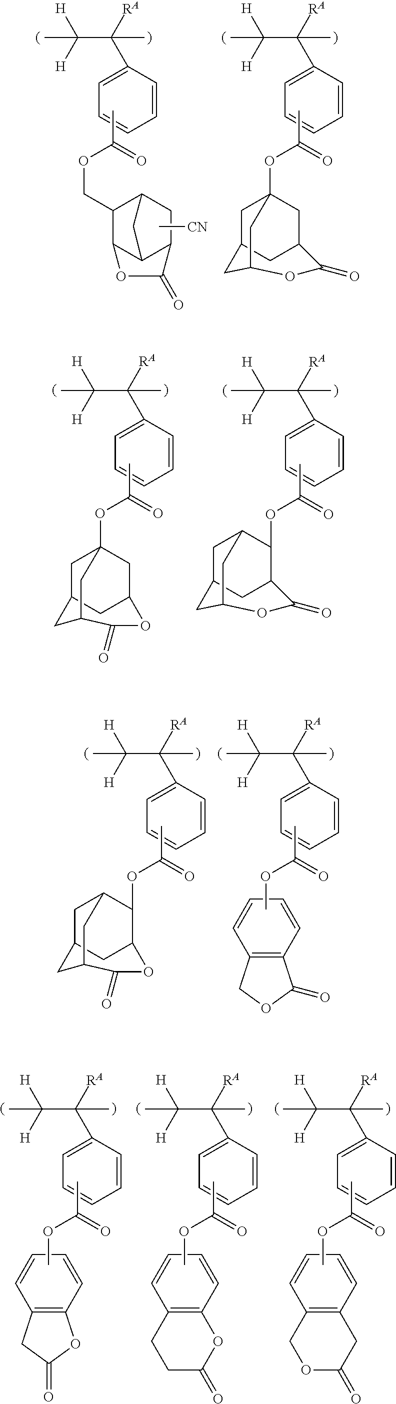

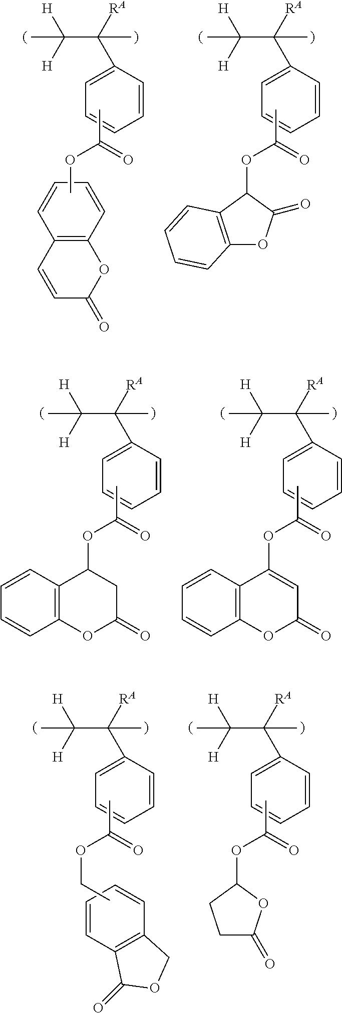

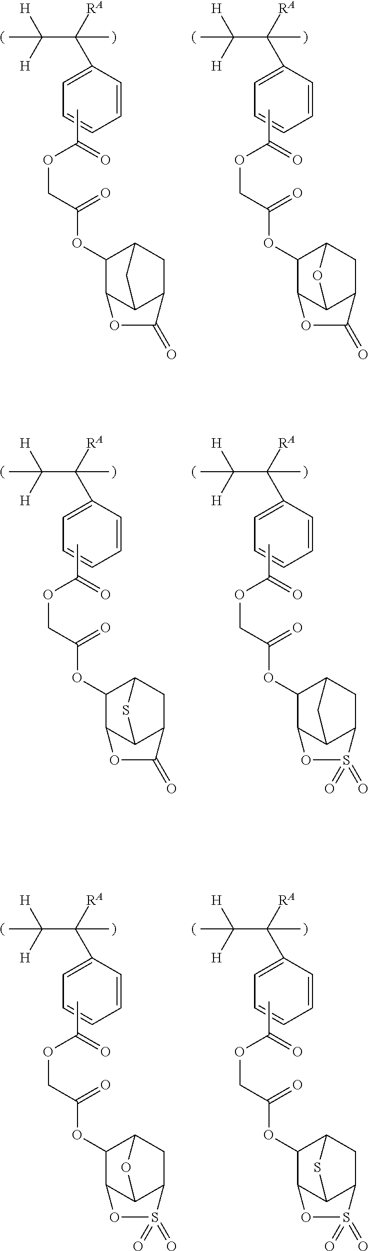

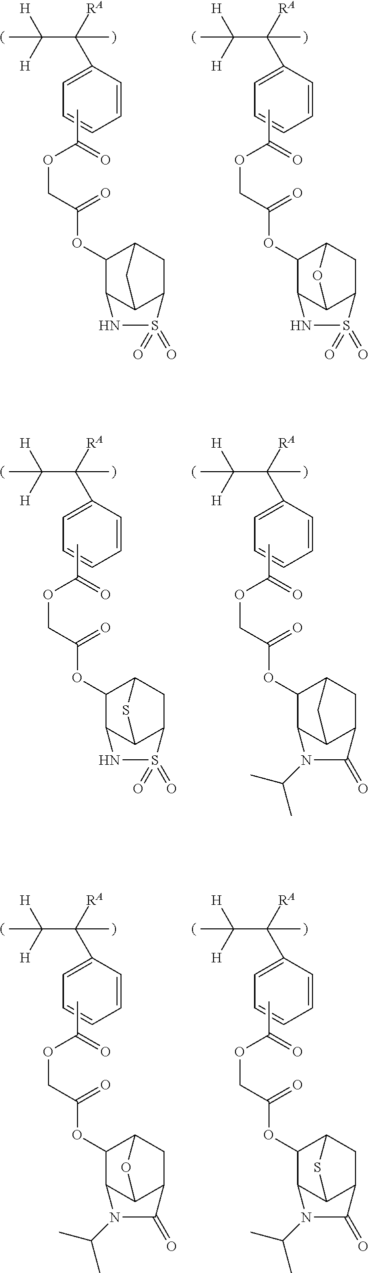

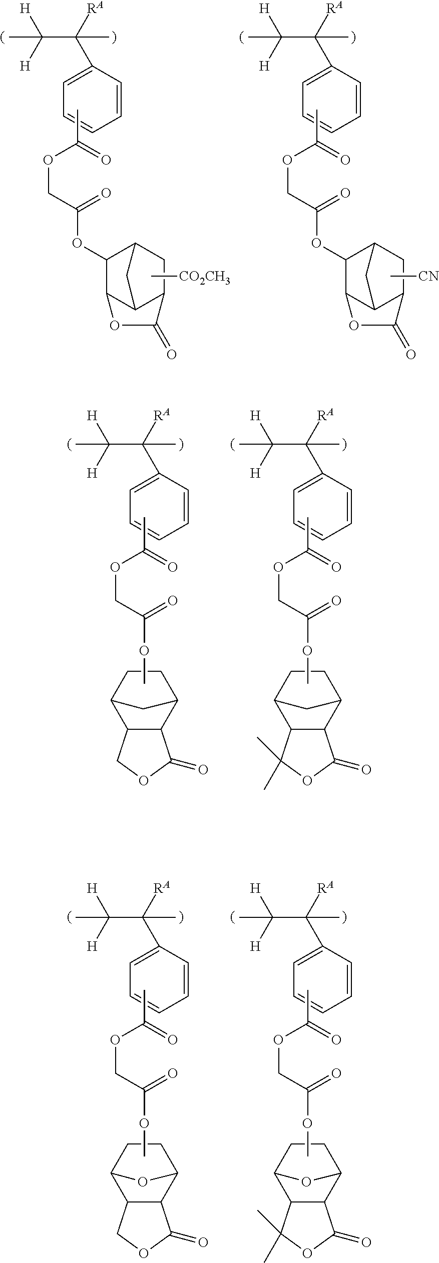

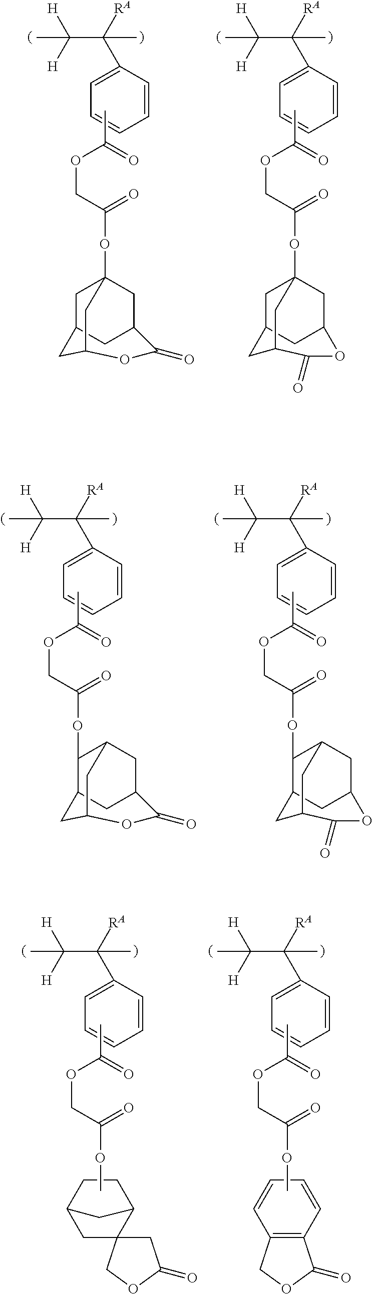

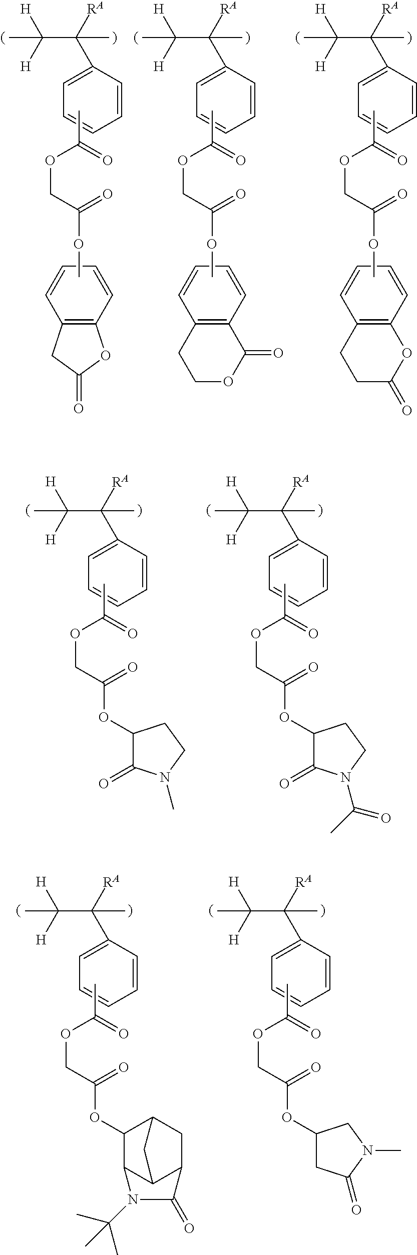

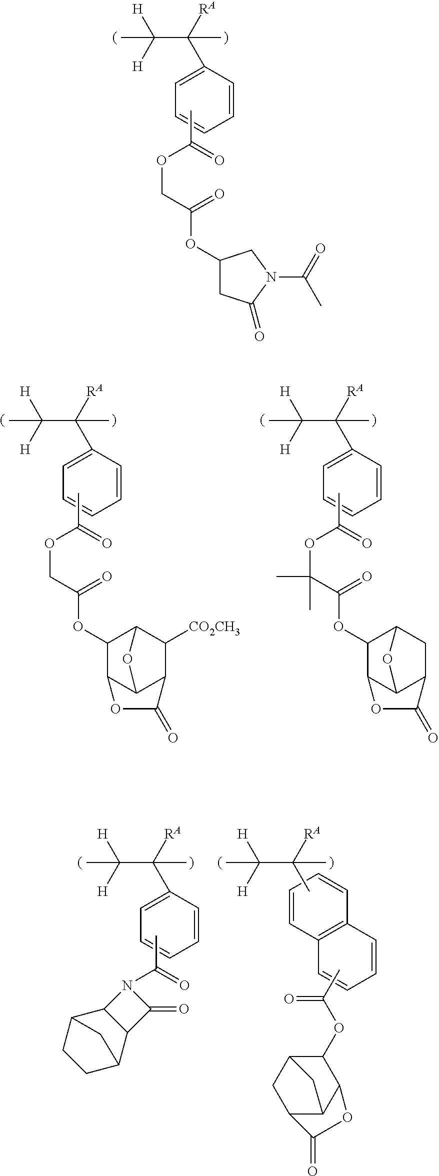

Examples of repeat unit b2 are shown below, but are not limited thereto. Herein, R^{A} is as defined above.

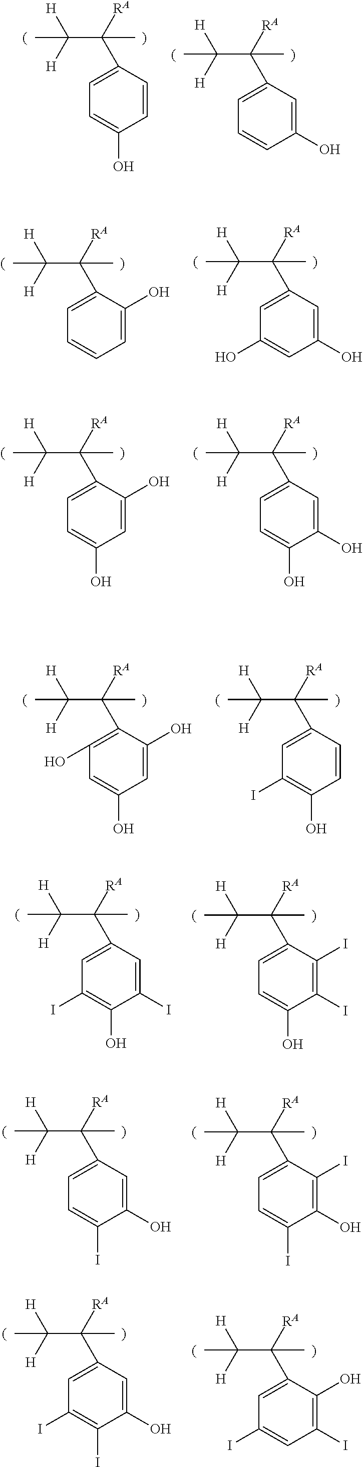

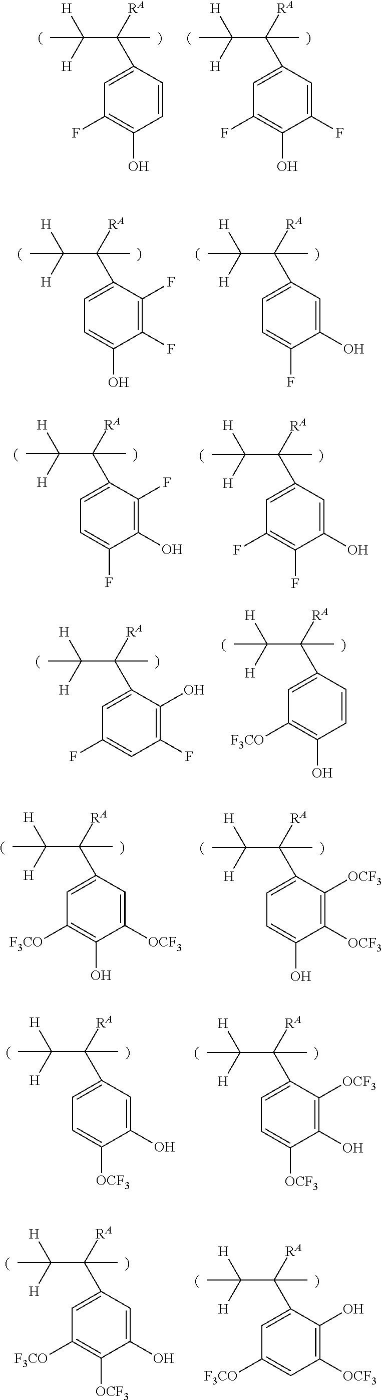

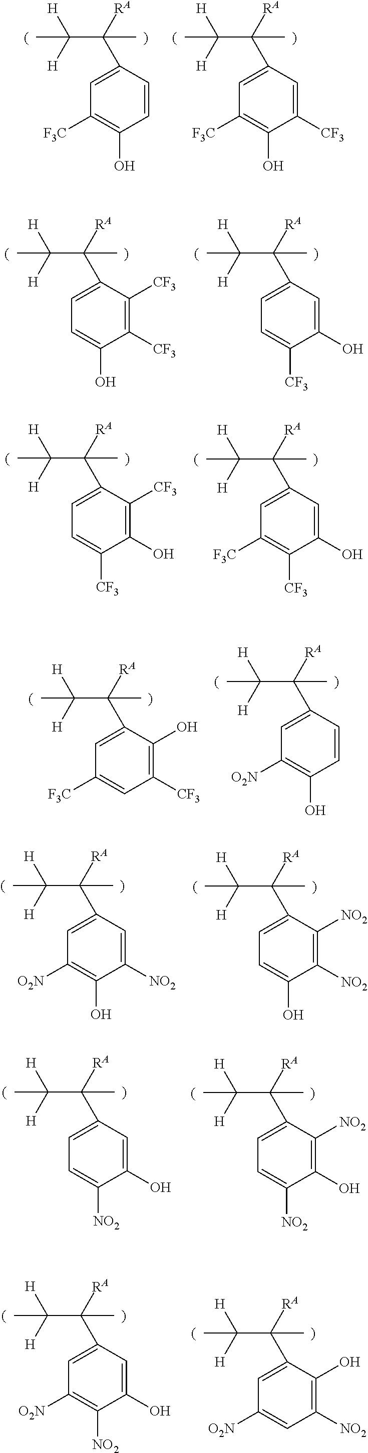

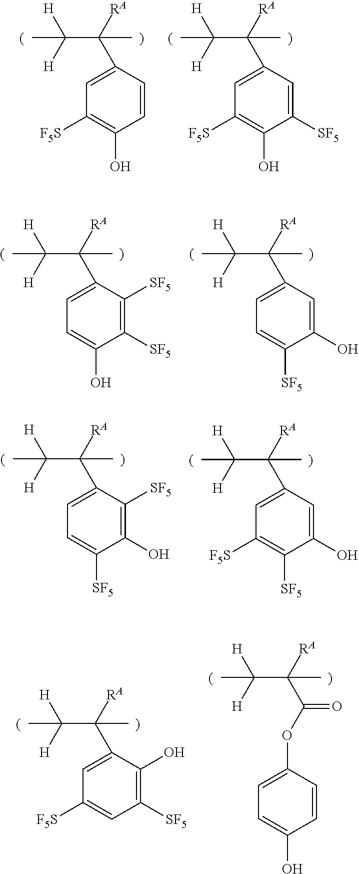

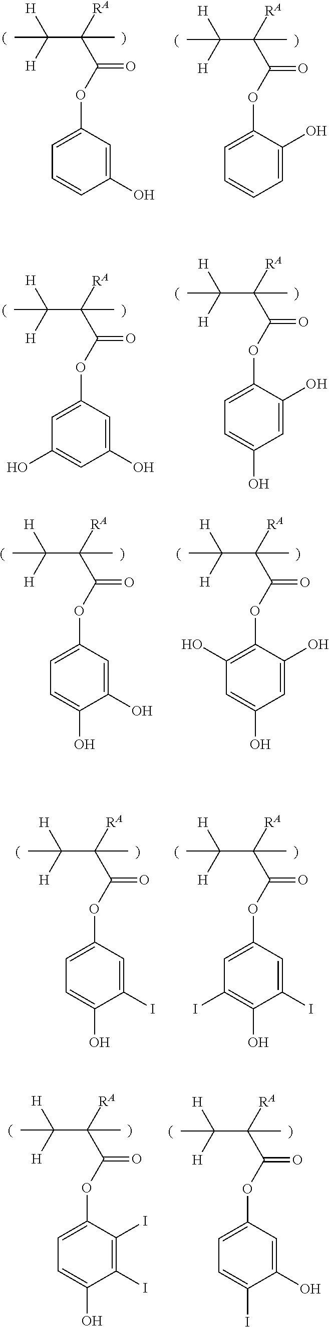

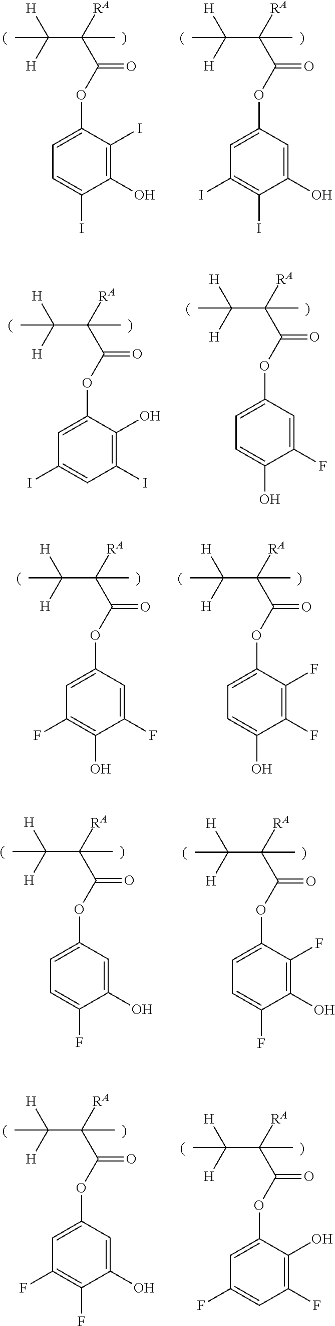

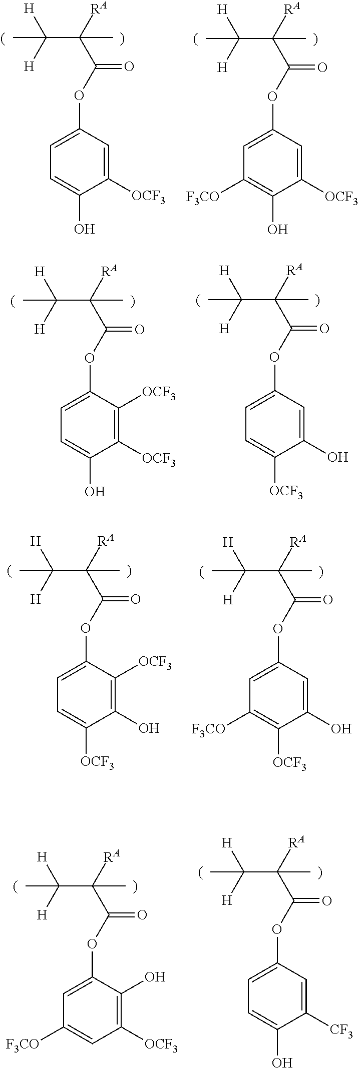

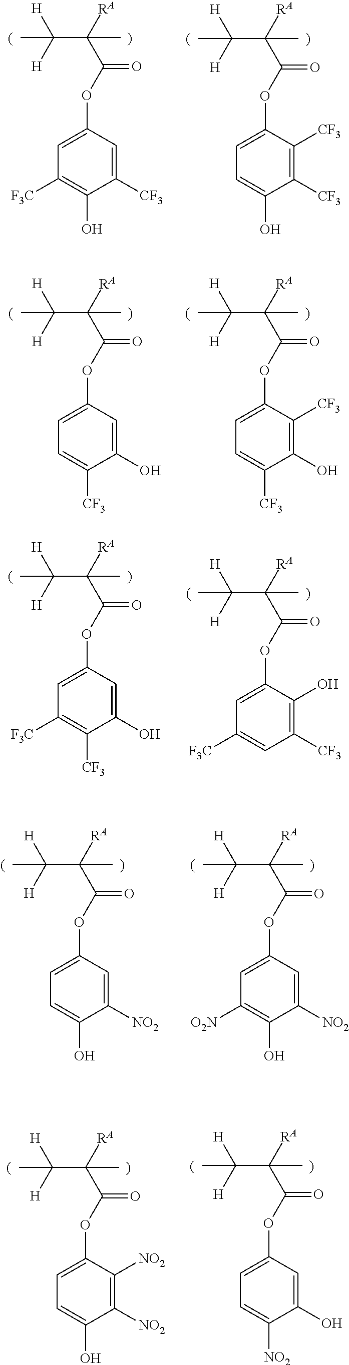

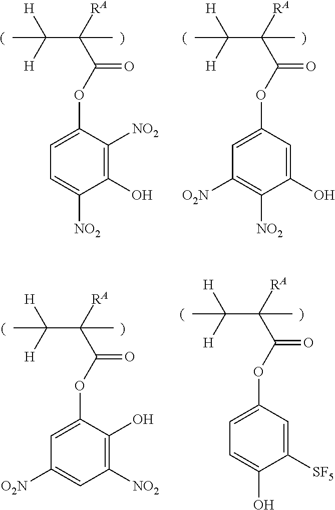

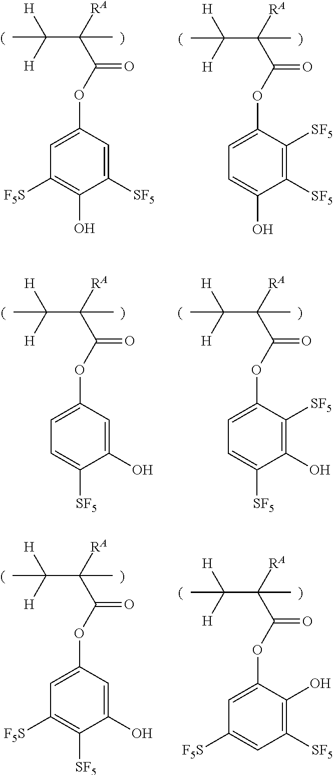

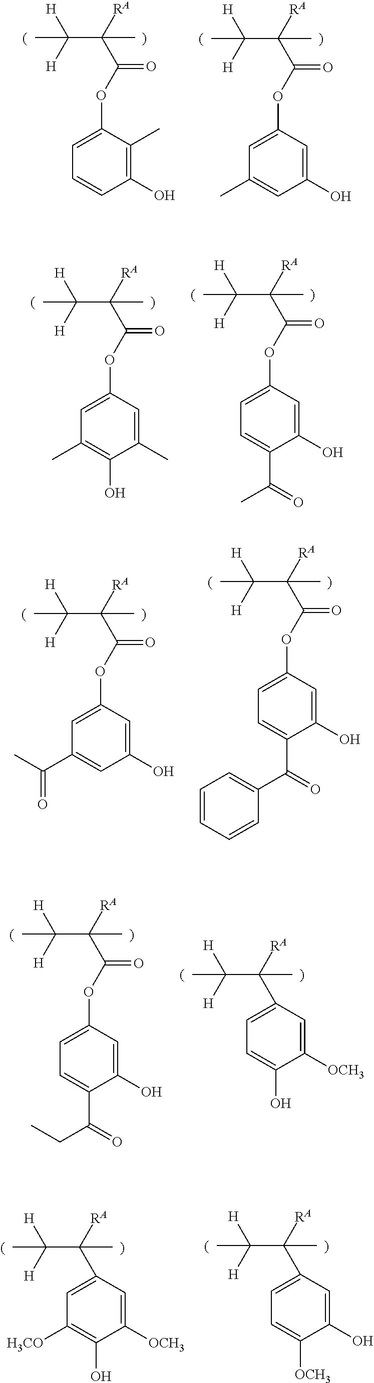

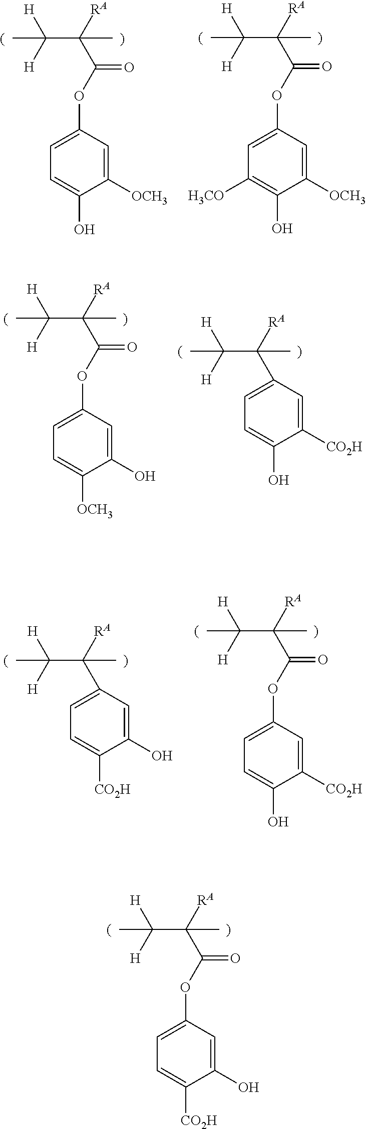

Of the repeat units b1 and b2, those units having a lactone ring as the polar group are preferred in the ArF lithography and those units having a phenolic site are preferred in the KrF, EB and EUV lithography.

The polymer may comprise repeat units of a structure having a hydroxy group protected with an acid labile group (hereinafter, also referred to as repeat units c). The repeat unit c is not particularly limited as long as the unit includes one or more structures having a hydroxy group protected with a protective group such that the protective group is decomposed to generate a hydroxy group under the action of acid. Repeat units having the formula (c1) are preferred.

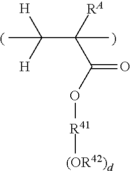

In formula (c1), R^{A} is hydrogen, fluorine, methyl or trifluoromethyl. R⁴¹ is a C₁-C₃₀ (d+1)-valent hydrocarbon group which may contain a heteroatom. R⁴² is an acid labile group. d is 1, 2, 3 or 4.

In formula (c1), the acid labile group R⁴² is deprotected under the action of acid so that a hydroxy group is generated. The structure of R⁴² is not particularly limited, an acetal structure, ketal structure, hydrocarbyloxycarbonyl group, and hydrocarbyloxymethyl group having the formula (c2) are preferred, with the hydrocarbyloxymethyl group having the formula (c2) being more preferred.

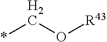

Herein, * designates a point of attachment. R⁴³ is a C₁-C₁₅ hydrocarbyl group.

Examples of the acid labile group R⁴², the hydrocarbyloxymethyl group having formula (c2), and the repeat units c are as exemplified for the repeat units c in JP-A 2020-111564.

The polymer may further comprise repeat units d derived from indene, benzofuran, benzothiophene, acenaphthylene, chromone, coumarin, and norbornadiene or derivatives thereof. Examples of the monomer from which the repeat units d are derived are shown below, but are not limited thereto.

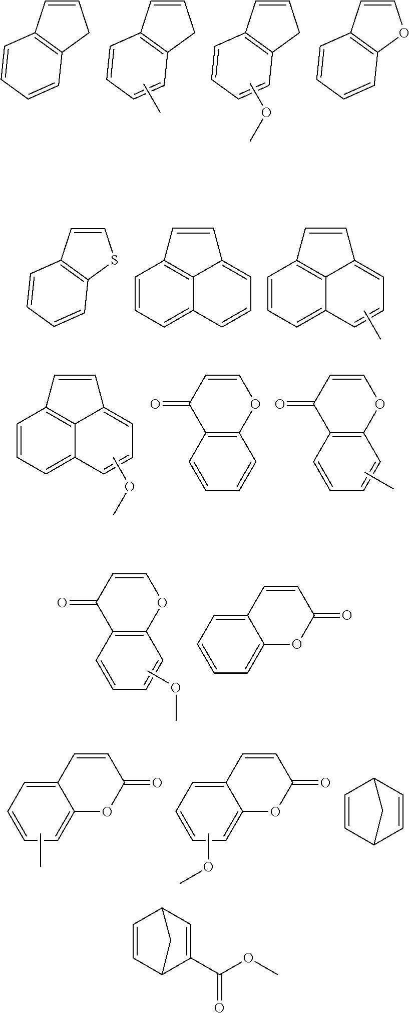

The polymer may comprise repeat units e derived from indane, vinylpyridine or vinylcarbazole.

In the polymer of the invention, a fraction of repeat units A, a1, a2, a3, b1, b2, c, d and e are preferably 0<A≤0.4, 0≤a1≤0.8, 0≤a2≤0.8, 0≤a3≤0.6, 0≤a1+a2+a3≤0.8, 0≤b1≤0.6, 0≤b2≤0.6, 0≤c≤0.5, 0≤d≤0.3, and 0≤e≤0.3, more preferably 0<A≤0.3, 0≤a1≤0.7, 0≤a2≤0.7, 0≤a3≤0.5, 0<a1+a2+a3≤0.7, 00≤b1≤0.5, 0≤b2≤0.5, 0≤c≤0.3, 0≤d≤0.3 and 0≤e≤0.3. Provided that A+a1+a2+a3+b1+b2+c+d+e 1.0.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. It is noted that Mw is as measured by GPC versus polystyrene standards using tetrahydrofuran (THF) or N,N-dimethylformamide (DMF) solvent.

Since the influence of dispersity (Mw/Mn) becomes stronger as the pattern rule becomes finer, the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight polymers and eliminates the risk of leaving foreign matter on the pattern or degrading the pattern profile after exposure and development.

Examples of the method for synthesizing the polymer include a method in which one or more monomers selected from the monomers corresponding to the foregoing repeat units are dissolved in an organic solvent, a radical polymerization initiator is added thereto, and the mixture is heated for polymerization.

Examples of the organic solvent which can be used for polymerization include toluene, benzene, tetrahydrofuran (THF), diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, and more preferably 2 to 12 hours from the aspect of production efficiency.

The polymerization initiator may be added to the monomer solution before supply to a reaction vessel, or an initiator solution may be prepared separately from the monomer solution and each solution may be supplied to a reaction vessel independently. Since there is a possibility that the initiator generates a radical in the standby time, by which polymerization reaction takes place to form an ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl, or similar group prior to polymerization, and the polymerization be followed by alkaline hydrolysis.

When hydroxystyrene or hydroxyvinylnaphthalene is copolymerized, hydroxystyrene or hydroxyvinylnaphthalene and other monomers may be dissolved in an organic solvent, a radical polymerization initiator is added thereto, and the mixture is heated for polymerization. Instead, as alternative method, acetoxystyrene or acetoxyvinylnaphthalene may be used and after polymerization, the acetoxy group is deprotected by alkaline hydrolysis, for thereby converting the polymer product to polyhydroxystyrene or hydroxypolyvinylnaphthalene.

Examples of the base that may be used in alkaline hydrolysis include aqueous ammonia and triethylamine. Preferably the reaction temperature is −20° C. to 100° C., more preferably 0° C. to 60° C. The reaction time is 0.2 to 100 hours, more preferably 0.5 to 20 hours.

The amounts of monomers in the monomer solution may be determined appropriate so as to provide the preferred fractions of repeat units as mentioned above.

It is described how to use the polymer obtained by the above preparation method. The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145]. Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and high-boiling alcohols such as diethylene glycol, propylene glycol, glycerol, 1,4-butanediol, and 1,3-butanediol, and mixtures thereof.

The polymer solution preferably has a polymer concentration of 0.01 to 30 wt %, more preferably 0.1 to 20 wt %.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be appropriately selected to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any number of times, in any order and in any stage. The reaction solution as polymerized or the polymer solution may be filtered, preferably both are filtered.

### [Chemically Amplified Resist Composition]

### (A) Base Polymer

A further embodiment of the invention is a chemically amplified resist composition comprising (A) a base polymer containing the polymer defined above.

The polymer defined above may be used alone or as a mixture of two or more polymers which are different in compositional ratio, Mw and/or Mw/Mn. In addition to the polymer, the base polymer (A) may contain a hydrogenated product of ring-opening metathesis polymerization (ROMP) polymer, which is described in JP-A 2003-066612.

### (B) Organic Solvent

The chemically amplified resist composition may comprise (B) an organic solvent. The organic solvent (B) is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixtures thereof.

Of the foregoing organic solvents, it is recommended to use 1-ethoxy-2-propanol, PGMEA, cyclohexanone, GBL, ethyl lactate, DAA, and mixtures thereof because the base polymer (A) is most soluble therein.

The organic solvent (B) in the chemically amplified resist composition is preferably added in an amount of 200 to 7,000 parts by weight, and more preferably 400 to 5,000 parts by weight per 80 parts by weight of the base polymer (A). The organic solvent (B) may be used alone or in admixture.

### (C) Quencher

The chemically amplified resist composition may comprise (C) a quencher. In the invention, the quencher refers to a compound capable of trapping the strong acid, which is generated by the photoacid generator in the chemically amplified resist composition upon light exposure, to prevent the acid from diffusing to the unexposed region and to assist in forming the desired pattern. The term “strong acid” means an acid having a sufficient acidity to induce deprotection reaction of an acid labile group.

Examples of the quencher (C) include onium salts having the formulae (1) and (2).

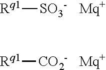

In the formula (1), R^{q1} is hydrogen atom or a C₁-C₄₀ hydrocarbyl group which may contain a heteroatom, exclusive of the hydrocarbyl group in which the hydrogen atom bonded to the carbon atom at α-position of the sulfo group is substituted by fluorine atom or fluoroalkyl group. In formula (2), R^{q2} is hydrogen or a C₁-C₄₀ hydrocarbyl group which may contain a heteroatom.

Examples of the C₁-C₄₀ hydrocarbyl group R^{q1} include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl and adamantyl groups; and C₆-C₄₀ aryl groups such as phenyl, naphthyl and anthracenyl groups. In the hydrocarbyl group, some or all hydrogen may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

Examples of the hydrocarbyl group R^{q2} include those exemplified above for R^{q1}, fluorinated saturated hydrocarbyl groups such as trifluoromethyl and trifluoroethyl groups, and fluorinated aryl groups such as pentafluorophenyl and 4-trifluoromethylphenyl groups.

Examples of the anion in the onium salt having formula (1) are shown below, but are not limited thereto.

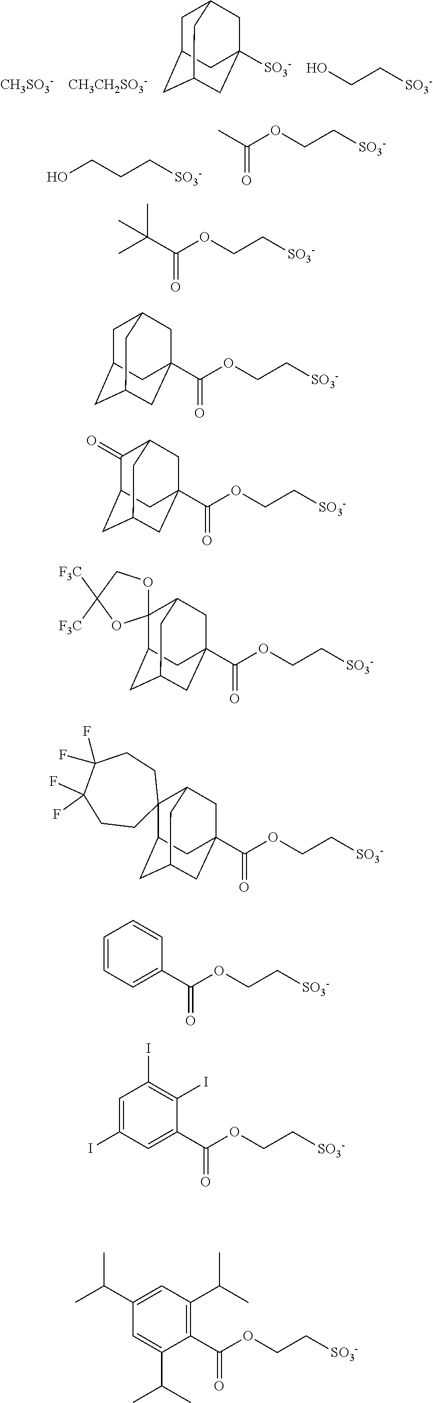

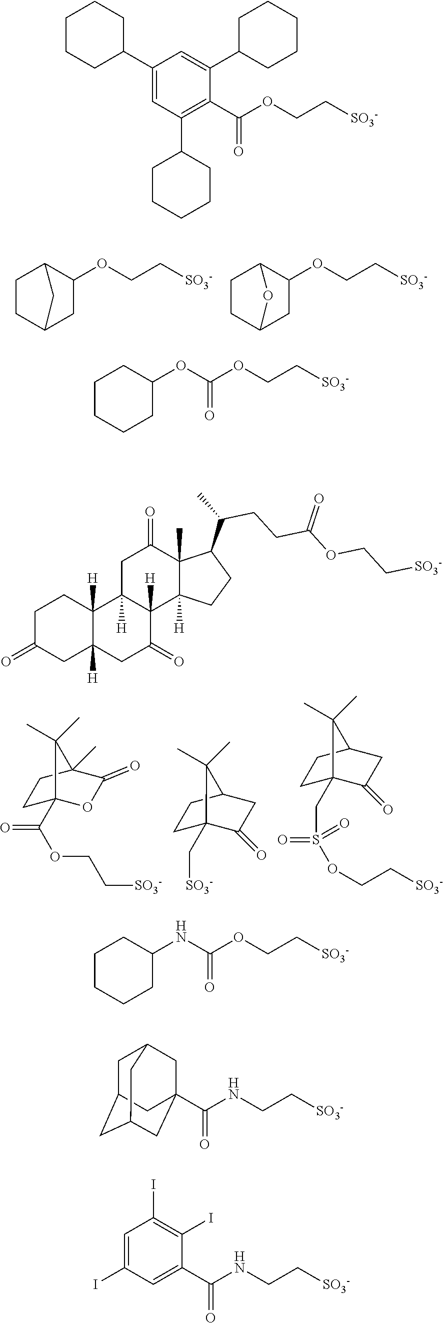

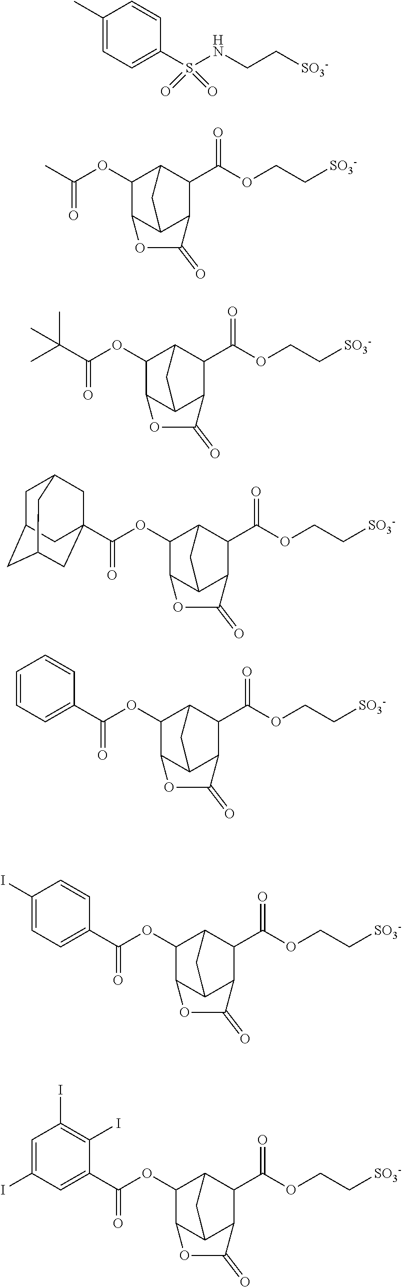

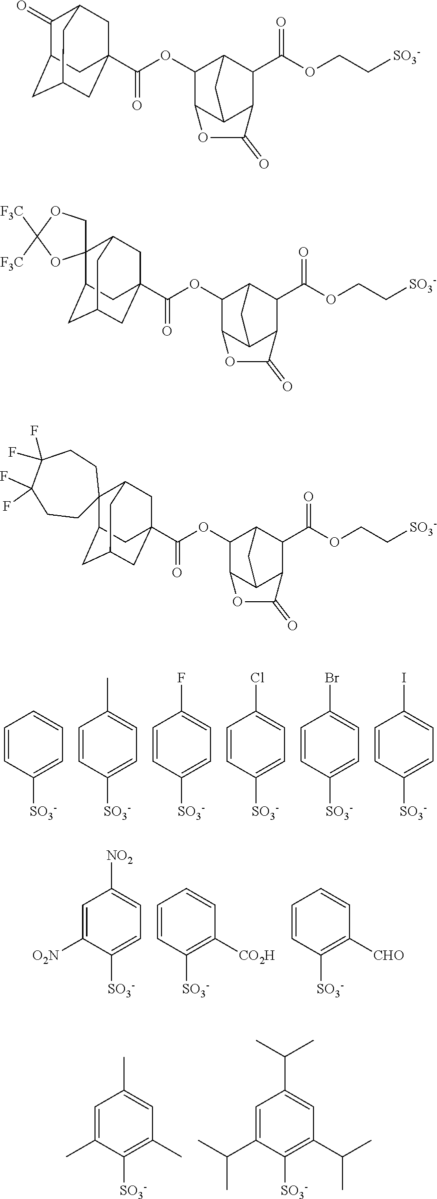

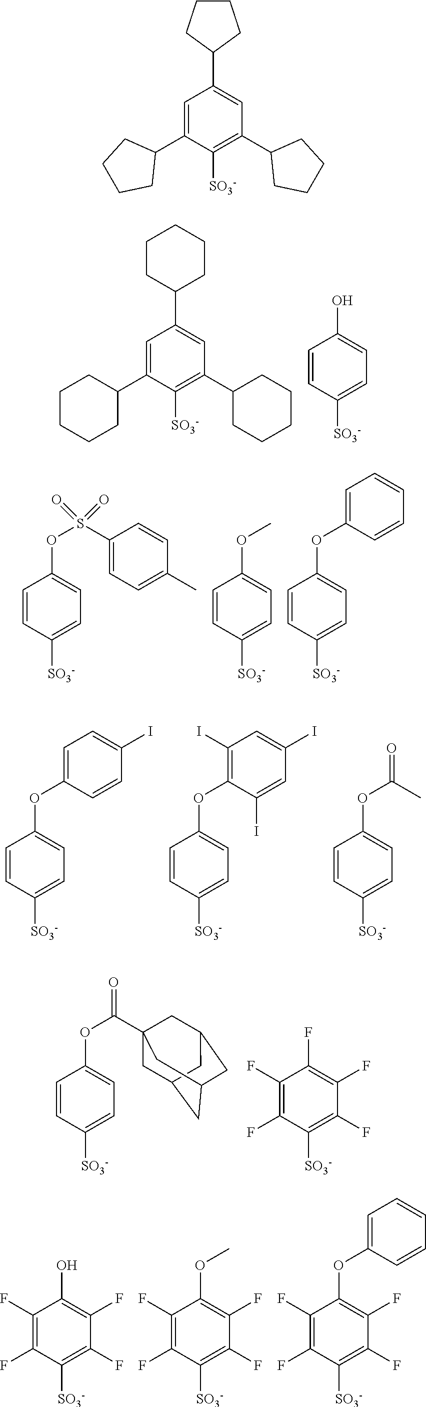

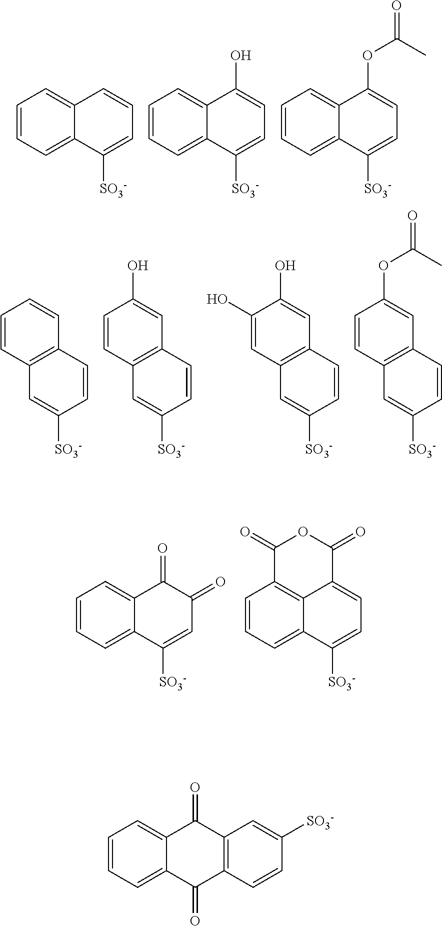

Other examples of the anion of the onium salt having formula (1) are as described in JP 6248882, paragraphs [0027]-[0029], JP 6648726, paragraphs [0029]-[0034], JP 7032549, paragraphs [0220]-[0225], JP 7067271, paragraphs [0028]-[0029], JP-A 2024-104830, paragraphs [0037]-[0060], JP-A 2023-177038, paragraphs [0039]-[0066], JP-A 2024-140135, paragraphs [0033]-[0093], and WO 2024/241820, paragraphs [0056]-[0059].

Examples of the anion in the onium salt having formula (2) are shown below, but are not limited thereto.

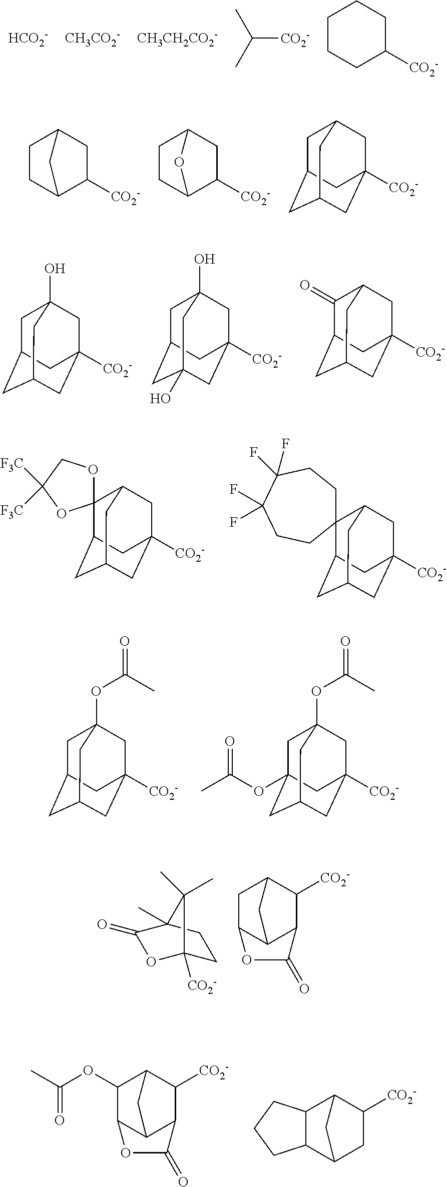

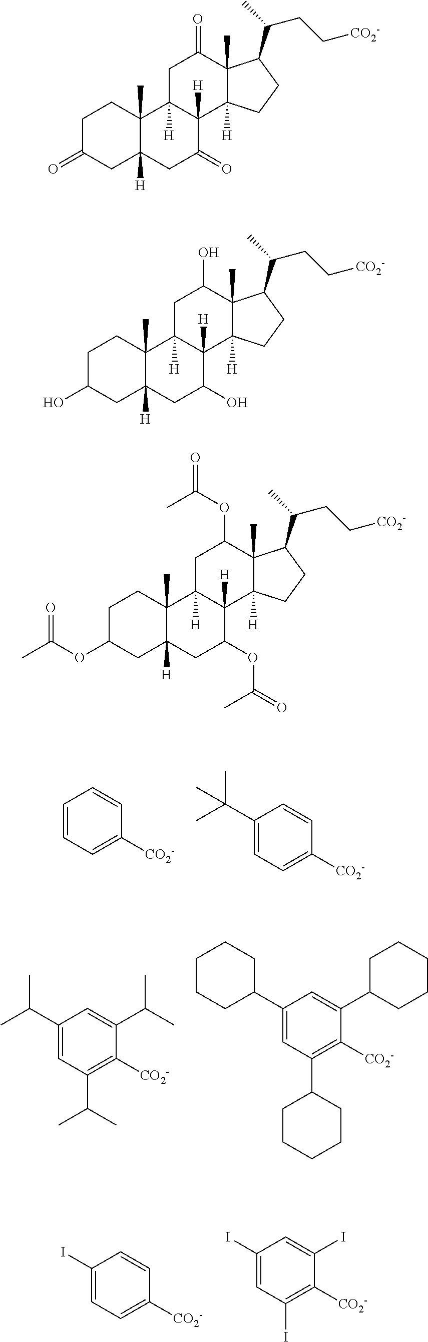

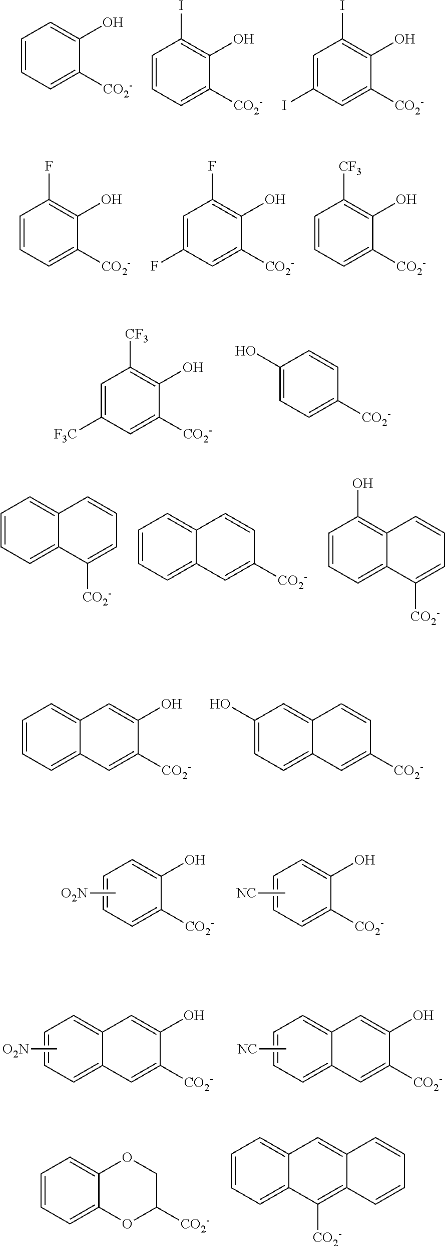

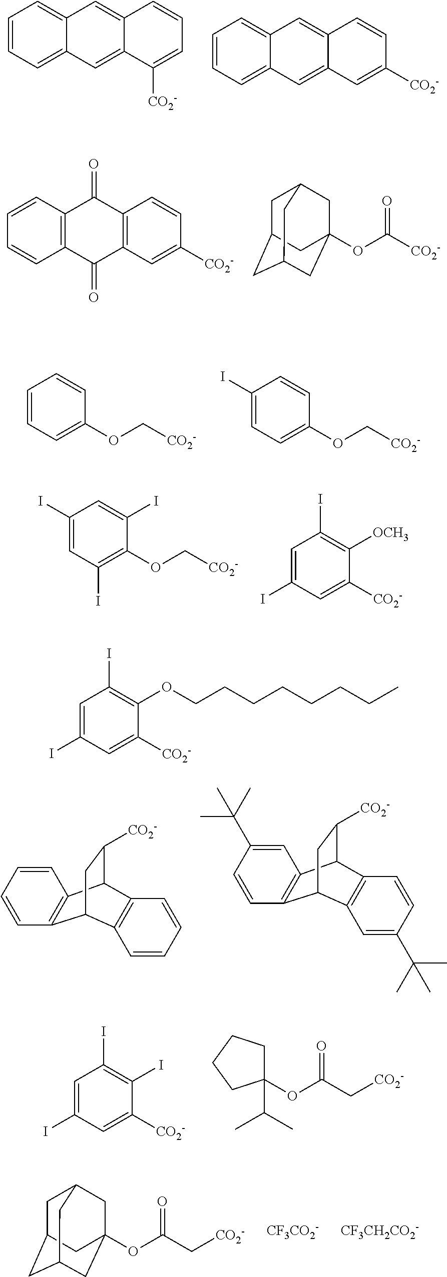

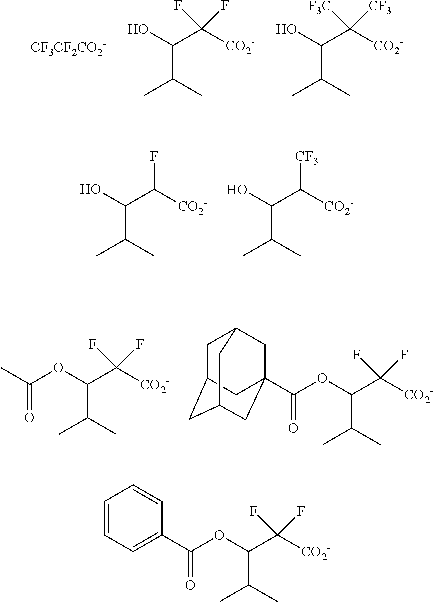

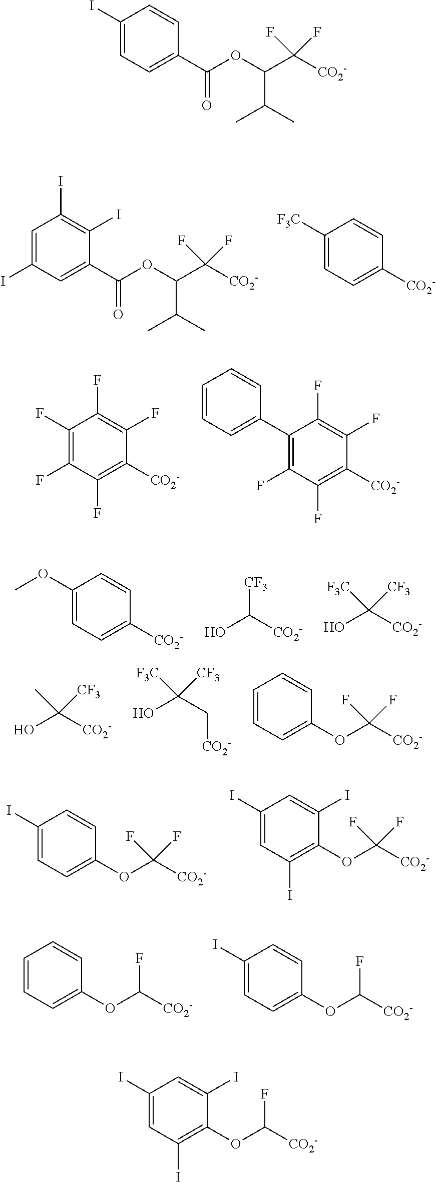

Other examples of the anion of the onium salt having formula (2) are as described in WO 2023/189502, paragraph [0097], WO 2023/199907, paragraphs [0231]-[0233] and [0236], WO 2023/223865, paragraphs [0240]-[0242], WO 2024/122425, paragraphs [0203]-[0211], WO 2024/166822, paragraphs [0282]-[0294], JP-A 2022-077505, paragraphs [0013]-[0015], JP-A 2022-091720, paragraphs [0022]-[0024], JP-A 2022-141598, paragraphs [0026]-[0031], JP-A 2023-018671, paragraphs [0017]-[0018], JP-A 2023-062898, paragraph [0157], JP-A 2023-108593, paragraph [0147], JP-A 2024-040793, paragraphs [0181]-[0194], JP-A 2024-067732, paragraph [0171], JP-A 2024-074267, paragraphs [0029]-[0030], JP-A 2024-074268, paragraph [0022], JP-A 2024-079160, paragraphs [0233]-[0237], JP-A 2024-083302, paragraphs [0025]-[0026], JP-A 2024-083305, paragraphs [0030]-[0031], JP-A 2024-084141, paragraphs [0032]-[0034], JP-A 2024-099498, paragraphs [0030]-[0031], JP-A 2024-099503, paragraphs [0032]-[0033], JP-A 2024-101558, paragraphs [0032]-[0033], JP-A 2024-102839, paragraphs [0027]-[0028], JP-A 2024-102841, paragraphs [0035]-[0036], JP-A 2024-113453, paragraphs [0256]-[0261], JP-A 2024-131484, paragraph [0266], JP-A 2025-010082, paragraphs [0022]-[0024], JP-A 2025-027484, paragraph [0299], JP 7611887, paragraphs [0190]-[0198], JP 7614747, paragraphs [0285]-[0290], and JP 7621323, paragraphs [0201]-[0209].

In formulae (1) and (2), Mq⁺ is an onium cation. The onium cation is preferably a sulfonium, iodonium or ammonium cation. Examples of the sulfonium cation are as exemplified for the cation in the sulfonium salt monomer in formula (A), and as described in JP-A 2024-003744, paragraphs [0102]-[0125], WO 2024/128017, paragraphs [0044]-[0049], and JP 7491173, paragraphs [0035]-[0046], but are not limited thereto.

As the sulfonium cation, sulfonium cations having the formula (sulfo-1) are also preferred.

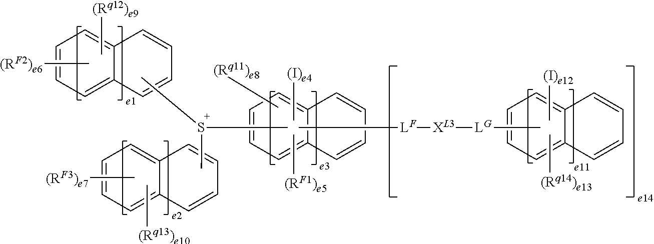

In formula (sulfo-1), e1 is 0 or 1. The relevant structure is a benzene ring when e1=0 and a naphthalene ring when e1=1. From the aspect of solvent solubility, the benzene ring corresponding to e1=0 is preferred. e2 is 0 or 1. The relevant structure is a benzene ring when e2=0 and a naphthalene ring when e2=1. From the aspect of solvent solubility, the benzene ring corresponding to e2=0 is preferred. e3 is 0 or 1. The relevant structure is a benzene ring when e3=0 and a naphthalene ring when e3=1. From the aspect of solvent solubility, the benzene ring corresponding to e3=0 is preferred.

In formula (sulfo-1), e4 is 0, 1, 2, 3 or 4. As the number of iodine atoms in the cation structure becomes larger, the amount of absorption of EUV increases, but precipitation in the resist composition may occur due to reduced solvent solubility. Therefore, e4 is preferably 0, 1, 2 or 3, more preferably 0, 1 or 2.

In formula (sulfo-1), e5 is 0, 1, 2, 3 or 4. It is preferred from the aspect of reactant availability that e5 be 0, 1, 2 or 3, more preferably 0, 1 or 2. e6 is 0, 1, 2, 3, 4, 5 or 6. It is preferred from the aspect of reactant availability that e6 be 0, 1, 2 or 3, more preferably 0, 1 or 2. e7 is 0, 1, 2, 3, 4, 5 or 6. It is preferred from the aspect of reactant availability that e7 be 0, 1, 2 or 3, more preferably 0, 1 or 2.

In formula (sulfo-1), e8 is 0, 1 or 2. From the aspect of reactant availability, e8 is preferably 0 or 1. e9 is 0, 1 or 2. From the aspect of reactant availability, e9 is preferably 0 or 1. e10 is 0, 1 or 2. From the aspect of reactant availability, e10 is preferably 0 or 1.

In formula (sulfo-1), e11 is 0 or 1. The sulfonium salt monomer has a benzene ring when e1 1=0, and a naphthalene ring when e11=1, and e11 is preferably 0 from the aspect of solvent solubility.

In formula (sulfo-1), e12 is 0, 1, 2, 3 or 4. As the number of iodine atoms in the cation structure becomes larger, the amount of absorption of EUV increases, but precipitation in the resist composition may occur due to reduced solvent solubility. Therefore, e12 is preferably 0, 1, 2 or 3, more preferably 0, 1 or 2.

In formula (sulfo-1), e13 is 0, 1 or 2. From the aspect of reactant availability, e13 is preferably 0 or 1. e14 is 0, 1 or 2. From the aspect of synthesis, e14 is preferably 0 or 1.

Provided that 0≤e6+e9≤4 when e1=0, and 0≤e6+e9≤6 when e1=1. 0≤e7+e10≤4 when e2=0, and 0≤e7+e10≤6 when e2=1. 1≤e4+e5+e8+e14≤4 when e3=0, and 1≤e4+e5+e8+e14≤6 when e3=1. 0≤e12+e13≤4 when e11=0, and 0≤e12+e13≤6 when e11=1. e4+e12≥1.

In formula (sulfo-1), R^{F1} to R^{F3} are each independently fluorine, a C₁-C₆ fluorinated saturated hydrocarbyl group, C₁-C₆ fluorinated saturated hydrocarbyloxy group or C₁-C₆ fluorinated saturated hydrocarbylthio group. Of these, trifluoromethyl, trifluoromethoxy, and trifluorothiomethoxy are preferred. When e5 is 2, 3 or 4, a plurality of R^{F1} may be identical or different. When e6 is 2, 3, 4, 5 or 6, a plurality of R^{F2} may be identical or different. When e7 is 2, 3, 4, 5 or 6, a plurality of R^{F3} may be identical or different.

In formula (sulfo-1), R^{g11} to R^{q14} are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom or C₁-C₂₀ hydrocarbylthio group which may contain a heteroatom. The hydrocarbyl group and hydrocarbyl moiety in the hydrocarbyloxy and hydrocarbylthio groups may be saturated or unsaturated, and may be straight, branched or cyclic. Examples thereof are as exemplified above for the hydrocarbyl groups R¹ to R³ in formula (A). In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety.

When e8=2, two R¹¹ may be identical or different and may bond together to form a ring with the carbon atoms to which they are attached. When e9=2, two R^{q12} may be identical or different and may bond together to form a ring with the carbon atoms to which they are attached. When e10=2, two R^{q13} may be identical or different and may bond together to form a ring with the carbon atoms to which they are attached. When e13=2, two R^{q14} may be identical or different and may bond together to form a ring with the carbon atoms to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur, or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—), or haloalkyl moiety.

The aromatic rings directly bonded to S⁺ in the sulfonium cation having formula (sulfo-1) may bond together to form a ring with S⁺. Exemplary structures of the ring are shown below.

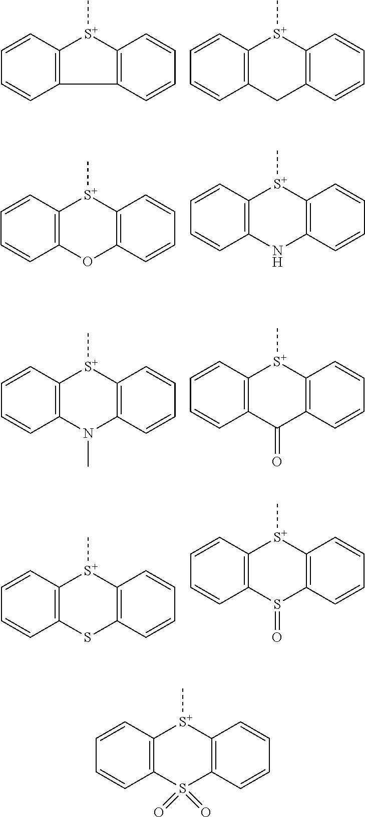

Herein, the broken line denotes a point of attachment.

In formula (sulfo-1), L^{F} and L^{G} are each independently a single bond, ether bond, ester bond, amide bond, sulfonate ester bond, sulfonamide bond, carbonate bond or carbamate bond. L^{F} is preferably a single bond, ether bond, ester bond, or sulfonate ester bond, more preferably an ester bond or sulfonate ester bond. L^{G} is preferably a single bond, ether bond, or ester bond, more preferably a single bond.

In formula (sulfo-1), X^{L3} is a single bond or a C₁-C₄₀ hydrocarbylene group which may contain a heteroatom. The hydrocarbylene group may be straight, branched or cyclic. Examples thereof include alkanediyl, cyclic saturated hydrocarbylene, and arylene groups. Suitable heteroatoms include oxygen, nitrogen, and sulfur. Examples of the optionally heteroatom-containing C₁-C₄₀ hydrocarbylene group X^{L3} are as exemplified above for the C₁-C₄₀ hydrocarbylene group represented by X^{L1} and X^{L2} in formula (Z2), specifically X^{L}-0 to X^{L}-61. Of these, X^{L}-0 to X^{L}-22, X^{L}-29 to X^{L}-34, and X^{L}-47 to X^{L}-61 are preferred as X^{L3}.

Preferably the sulfonium cation having formula (sulfo-1) has the formula (sulfo-1-1).

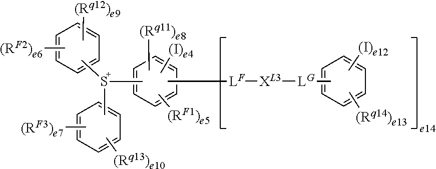

Herein, e4 to e10, e12 to e14, R^{F1} to R^{F3}, R¹¹ to R^{q14}, L^{F}, L^{G}, and X^{L3} are as defined above.

More preferably the sulfonium cation having formula (sulfo-1-1) has the formula (sulfo-1-2).

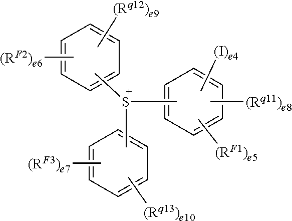

Herein, e4 to e10, R^{F1} to R^{F3}, and R^{q11} to R^{q13} are as defined above.

Examples of the sulfonium cation having formula (sulfo-1) are shown below, but are not limited thereto. Herein, Me is methyl.

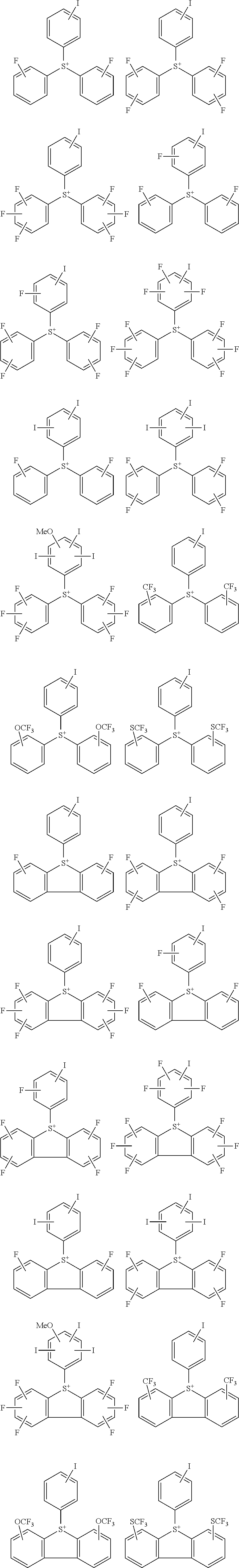

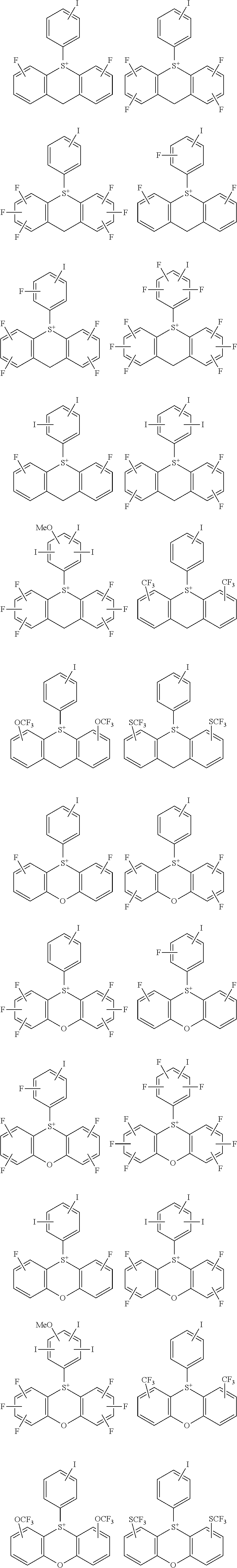

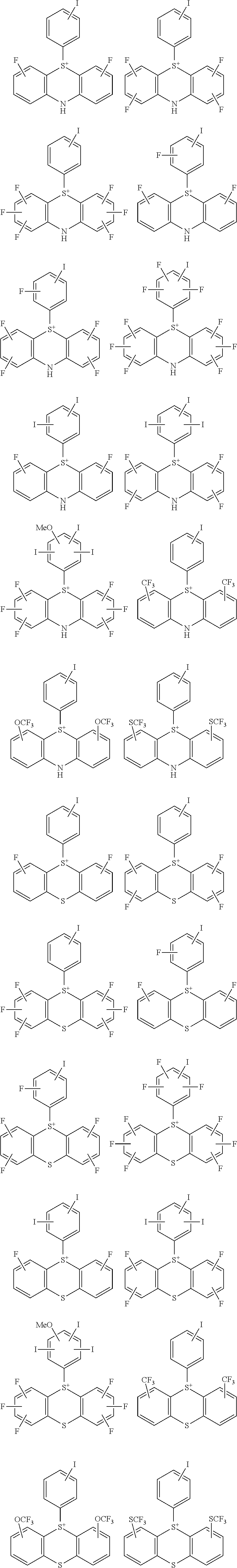

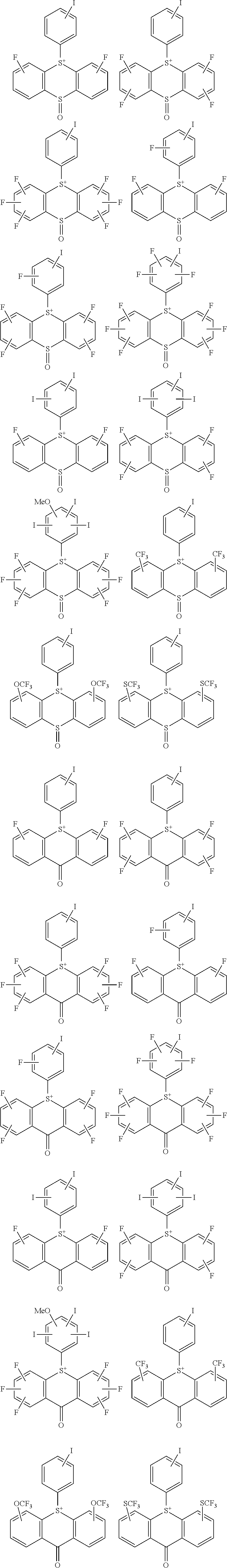

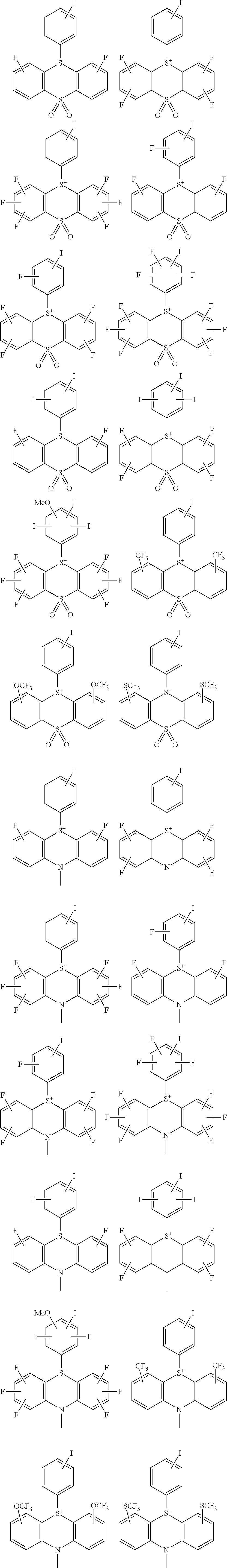

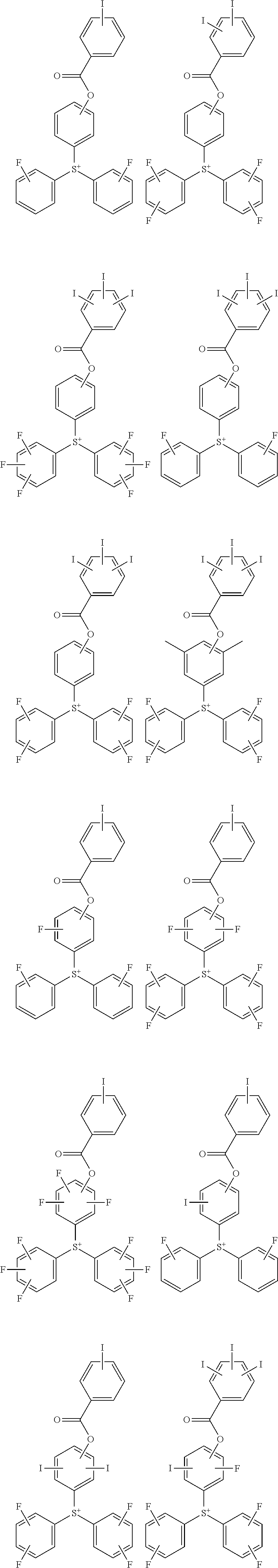

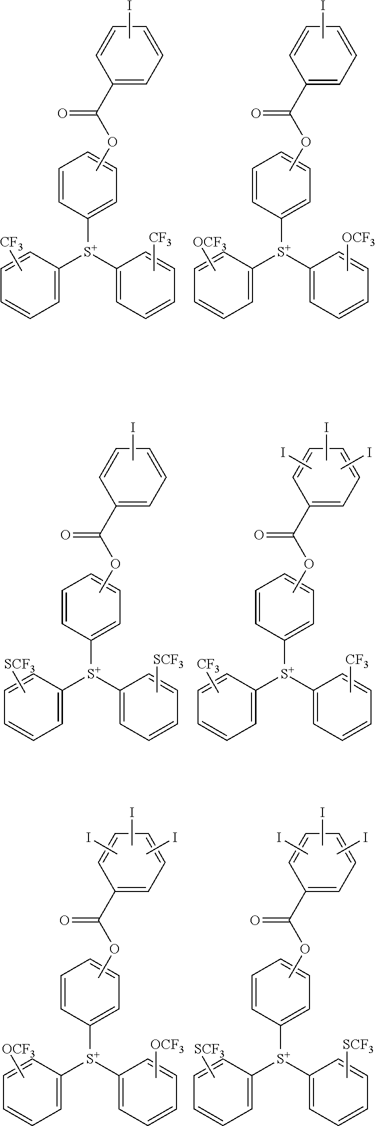

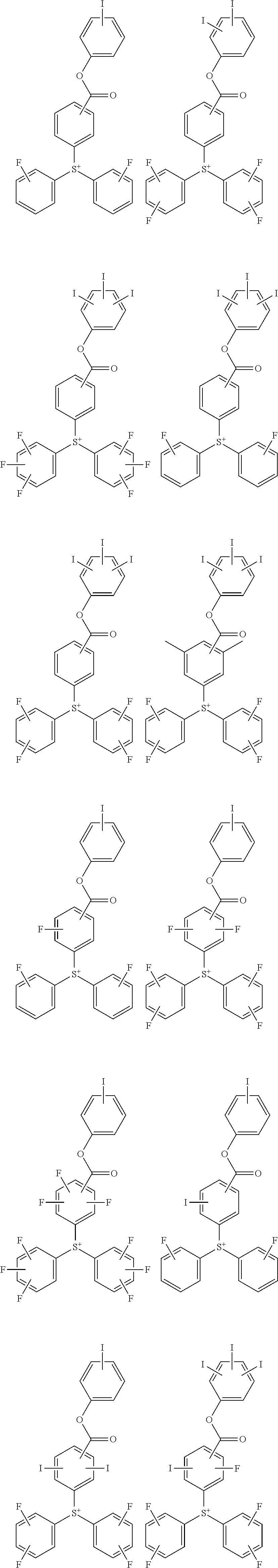

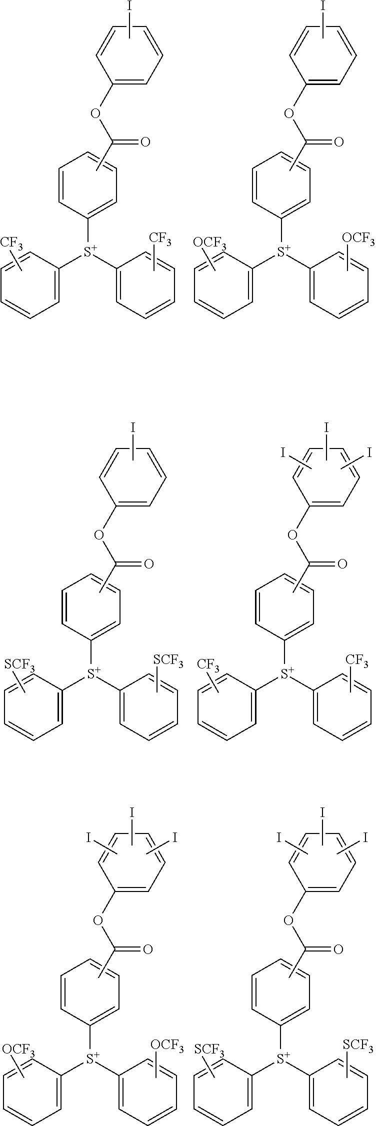

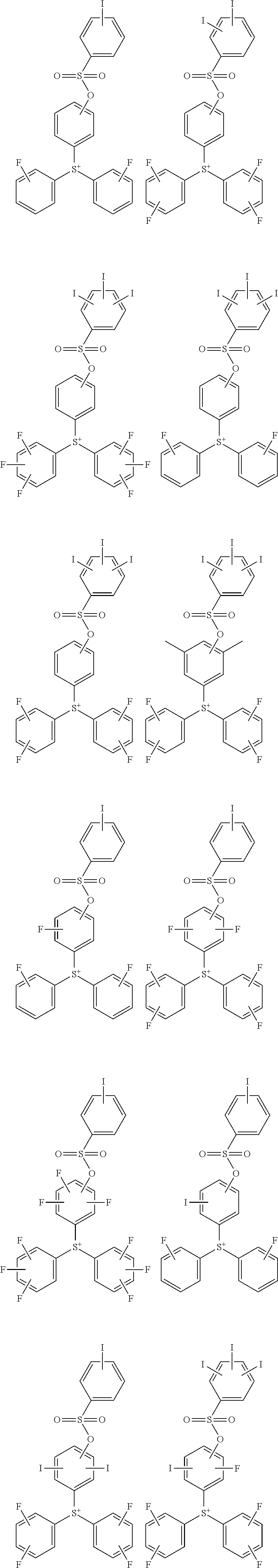

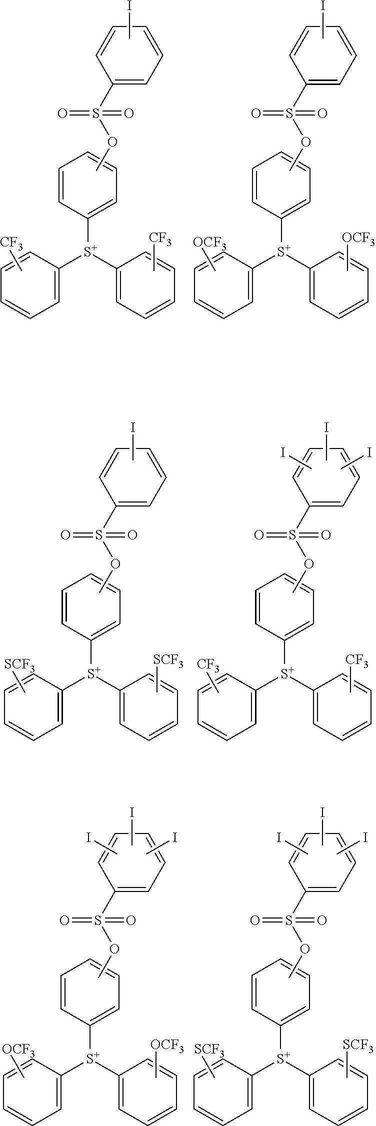

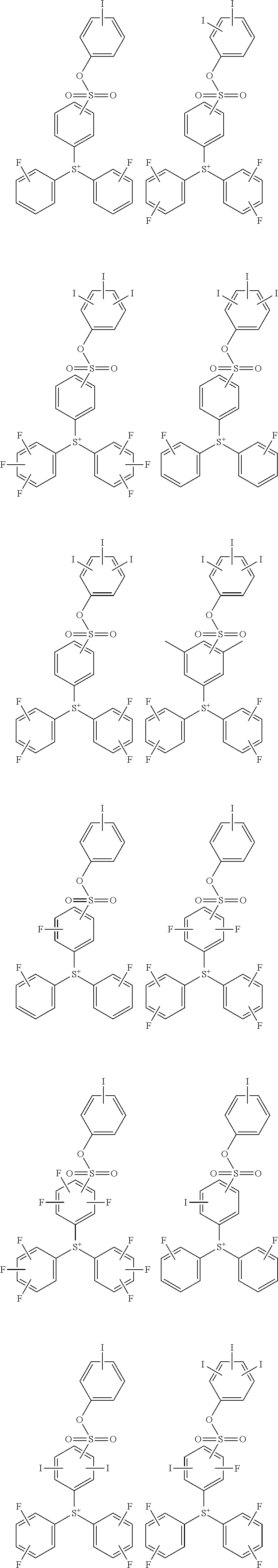

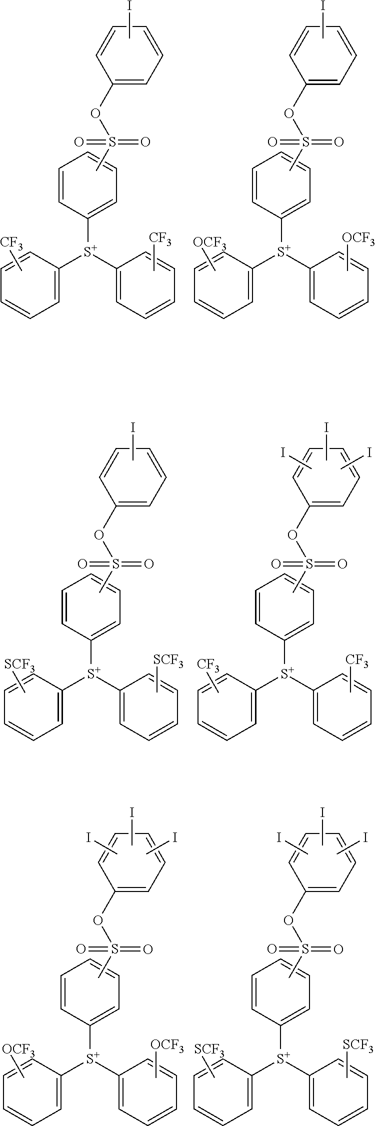

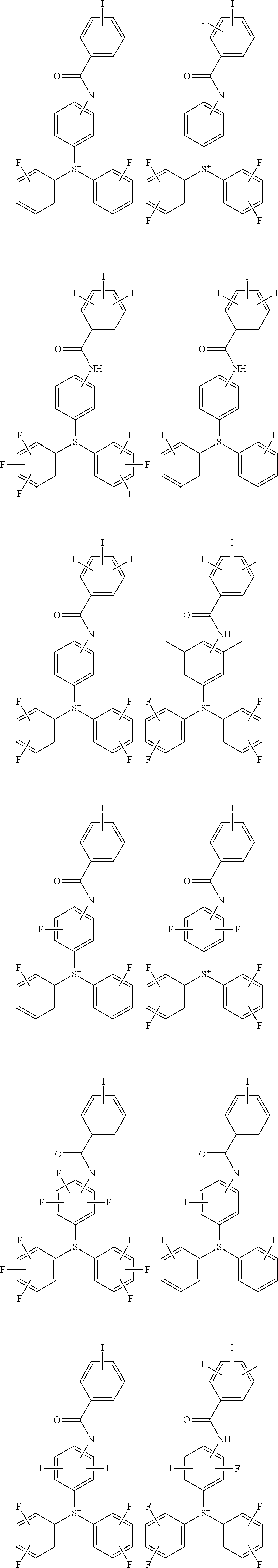

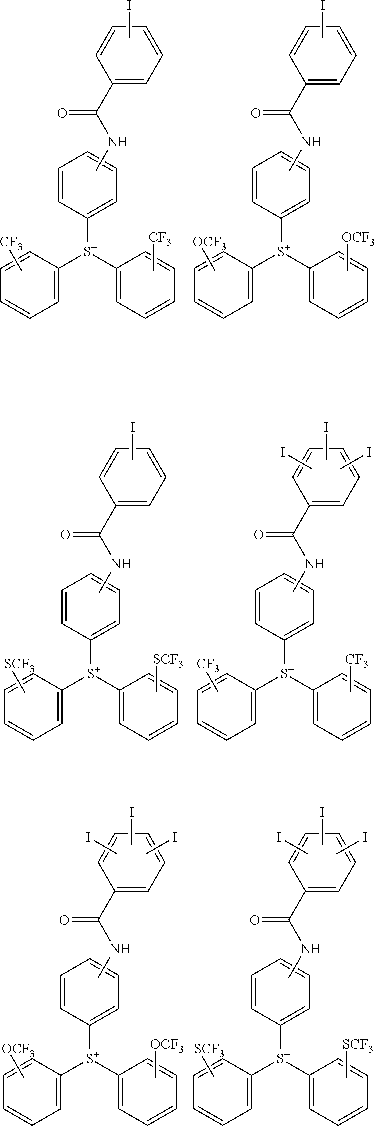

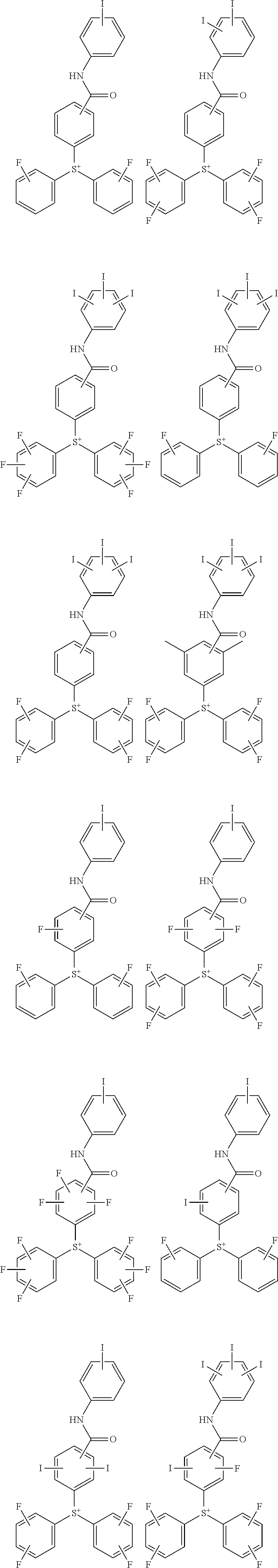

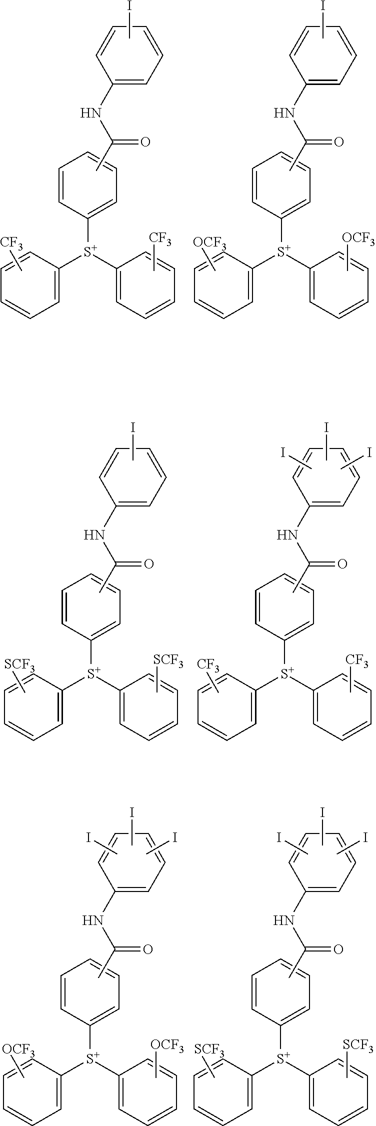

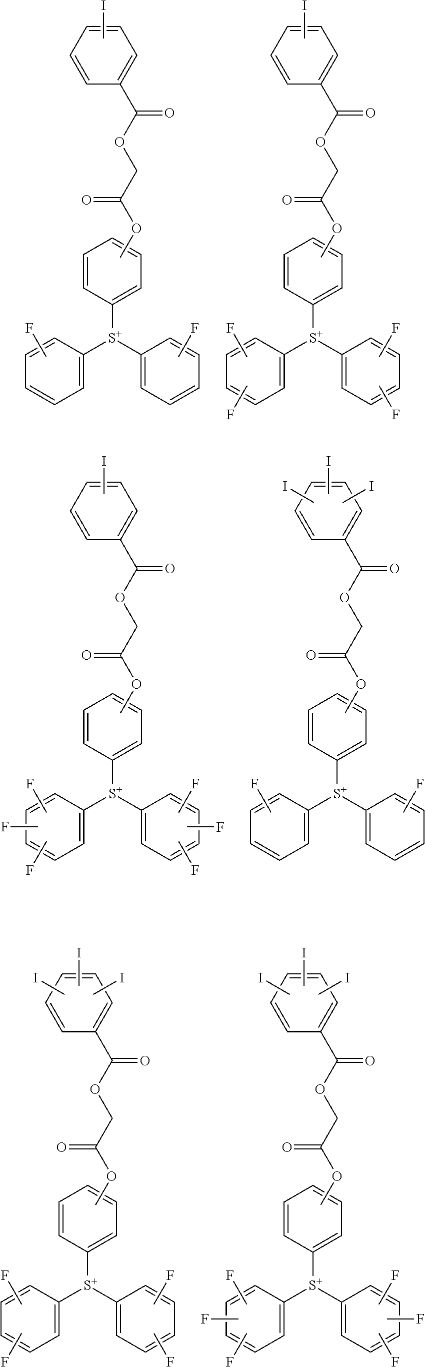

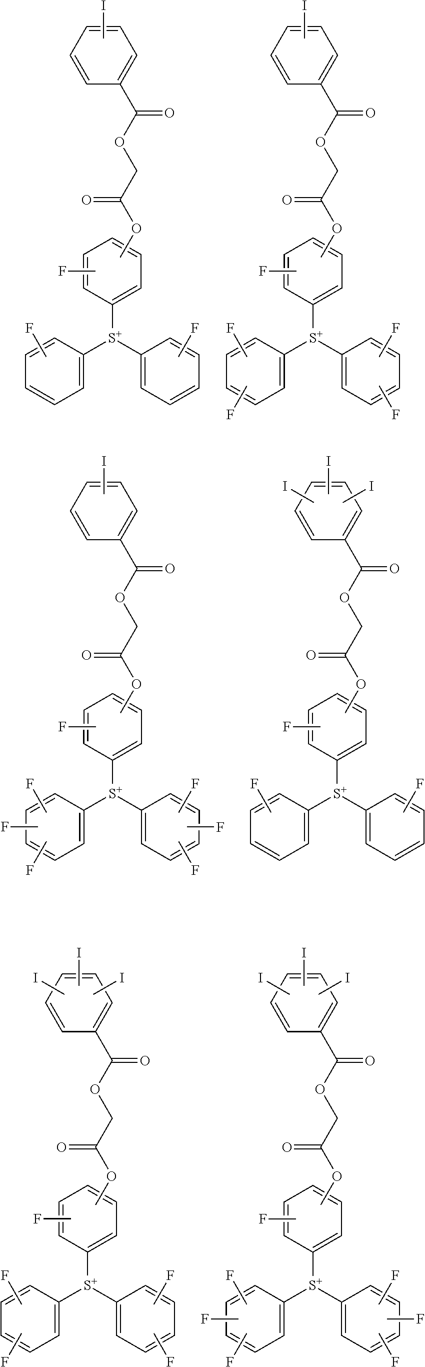

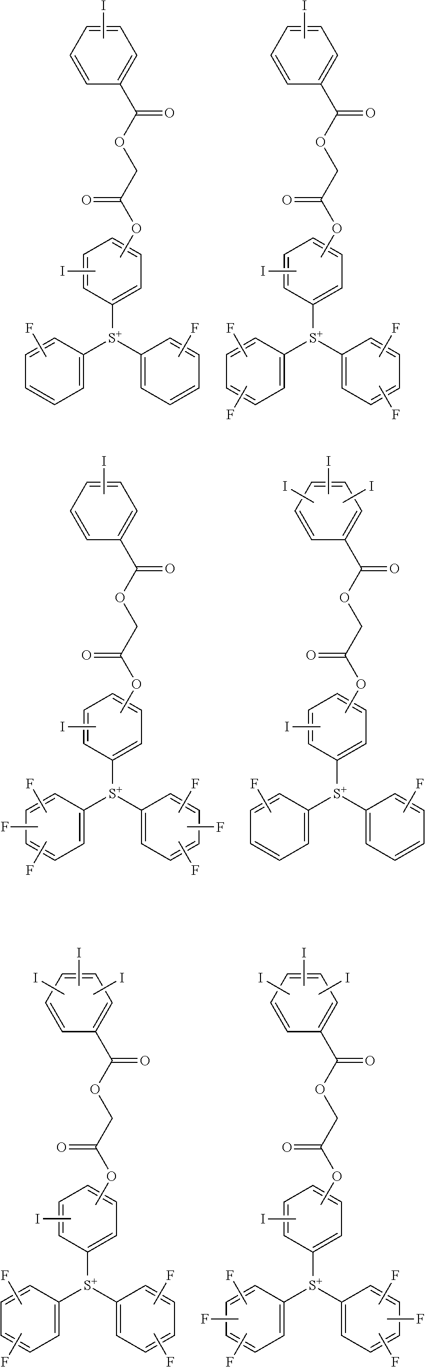

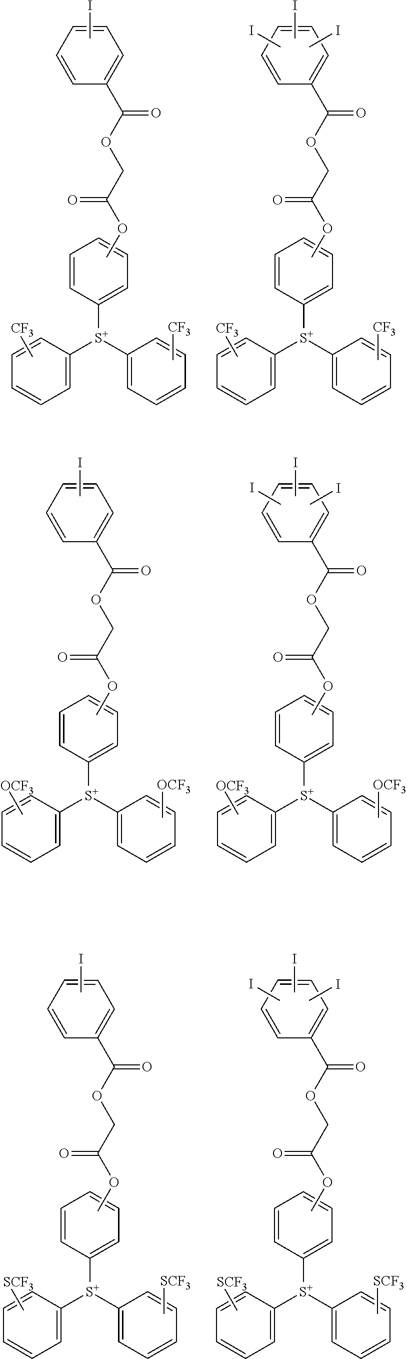

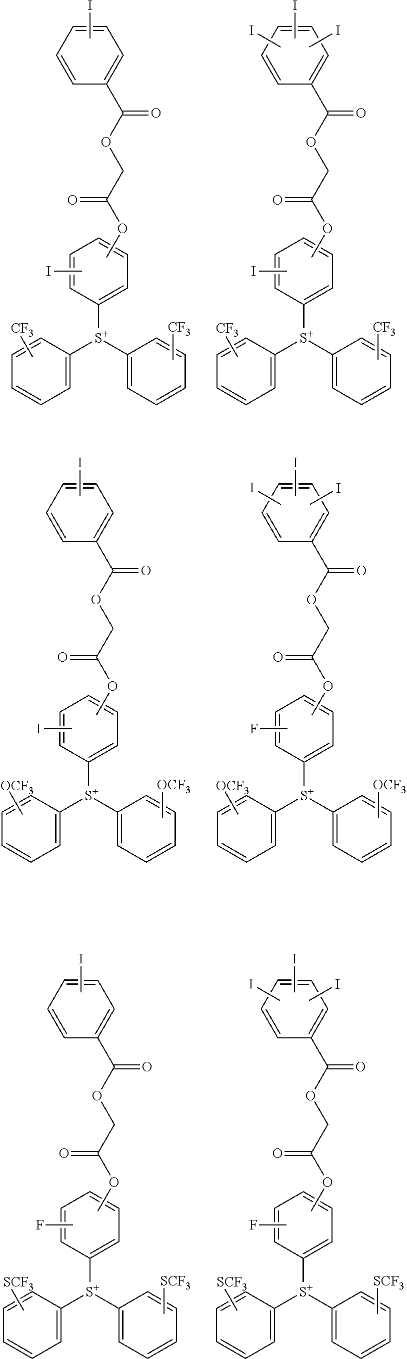

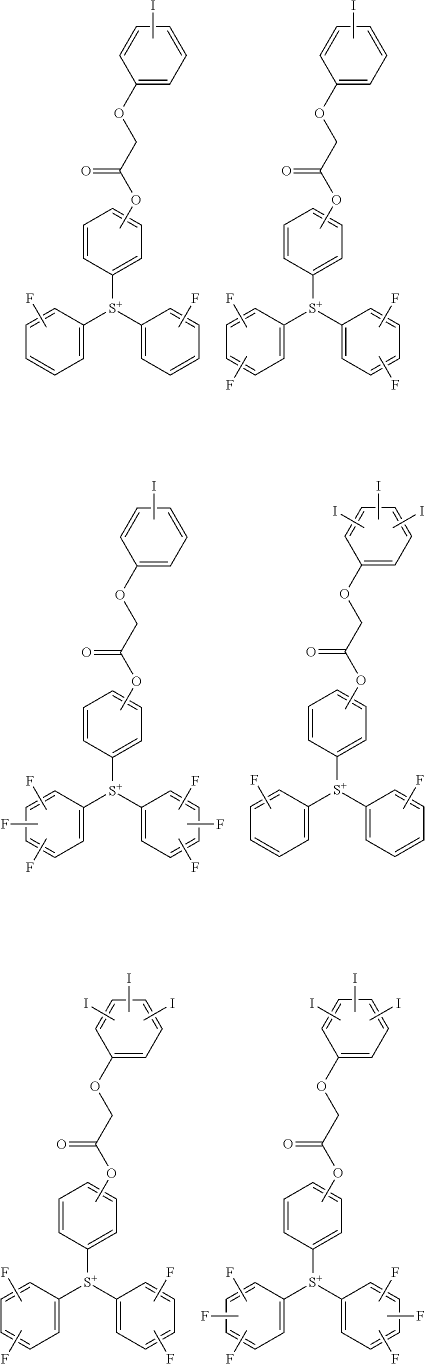

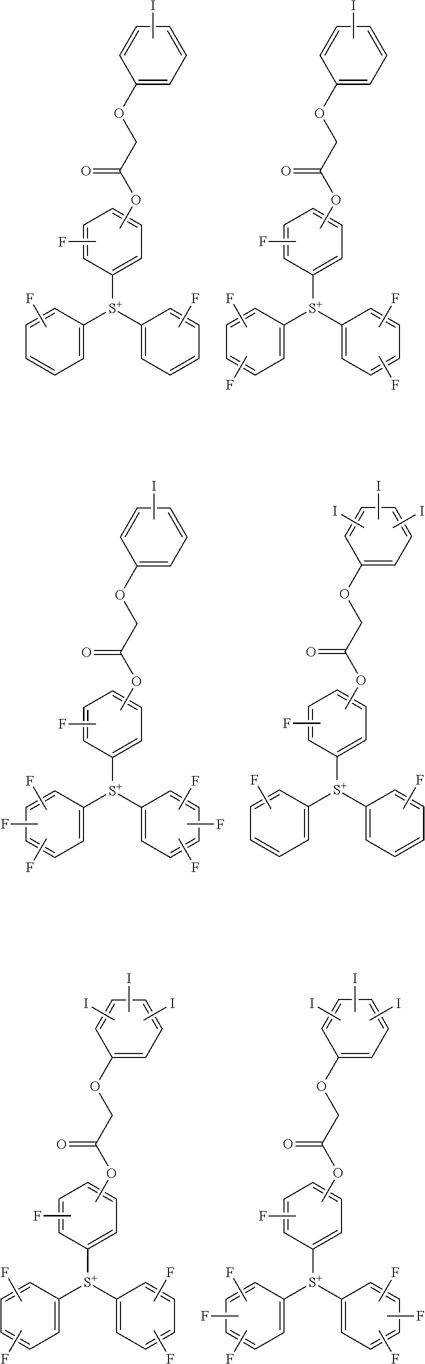

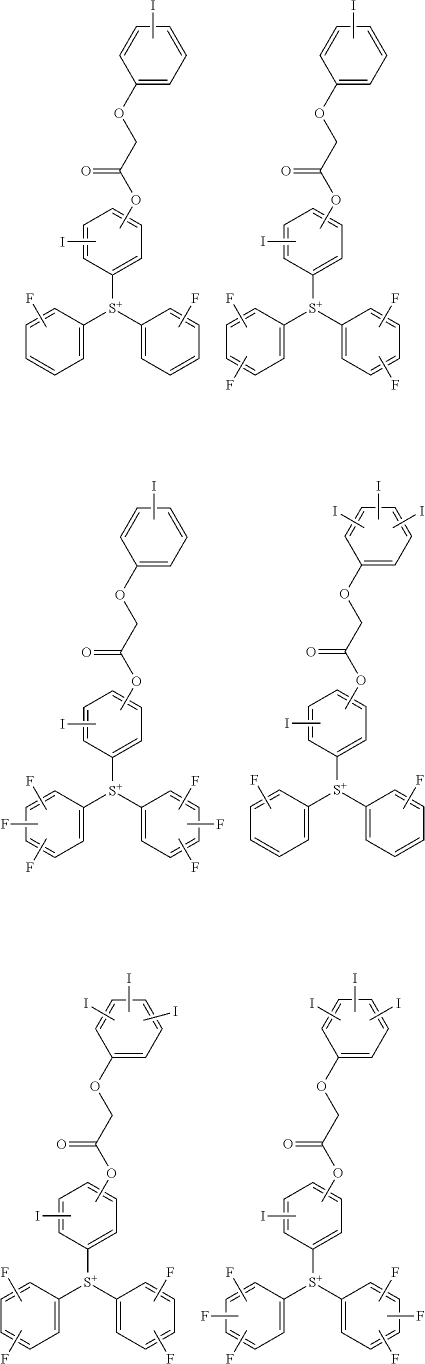

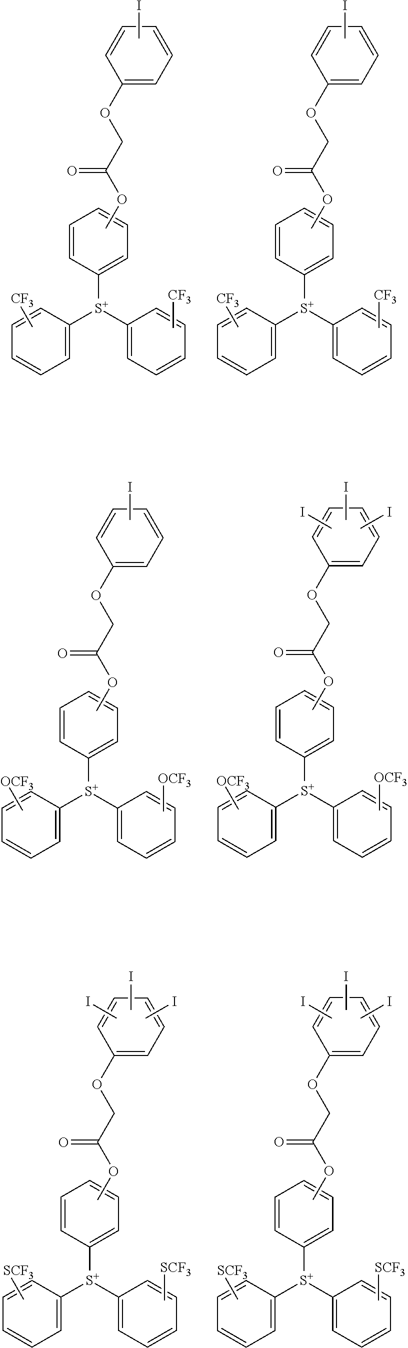

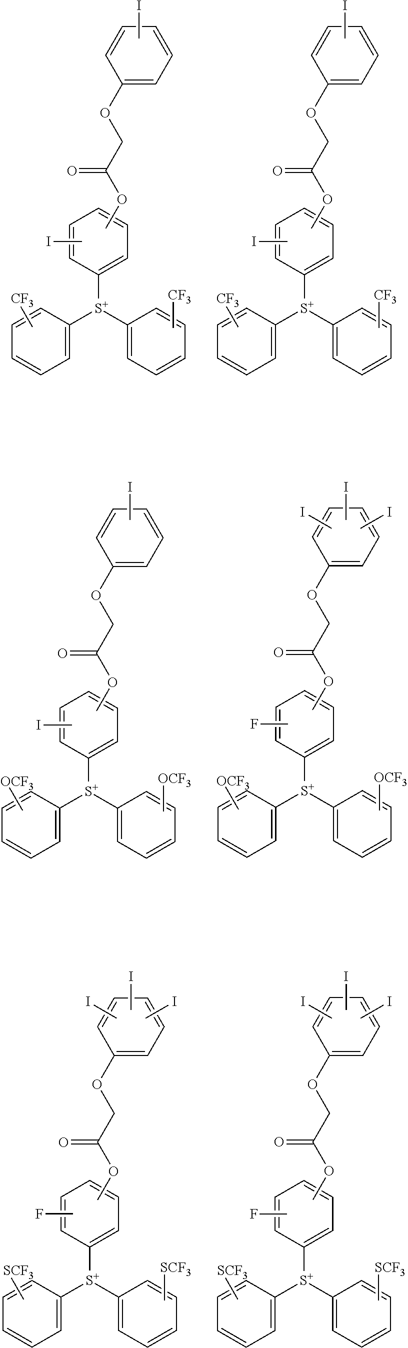

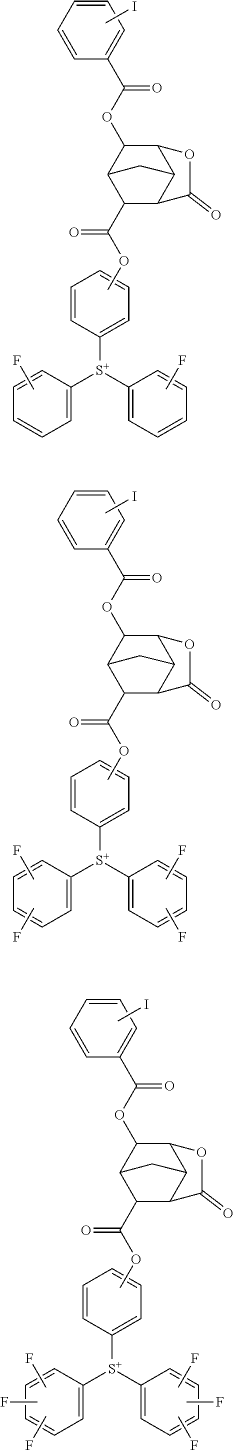

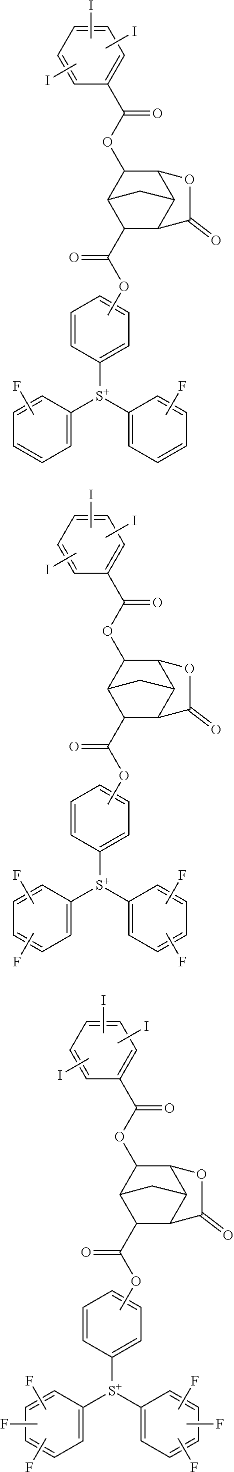

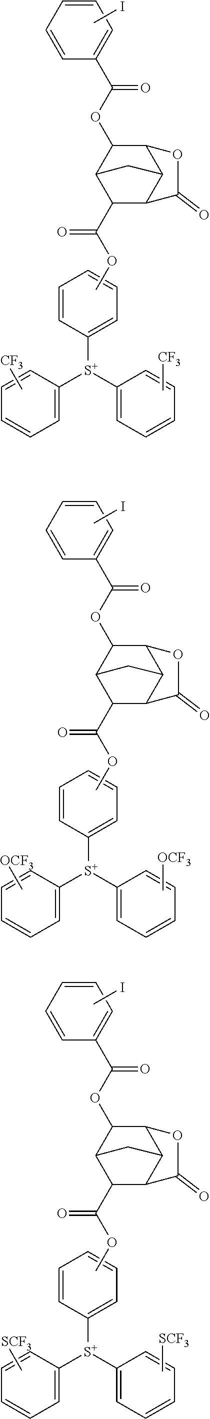

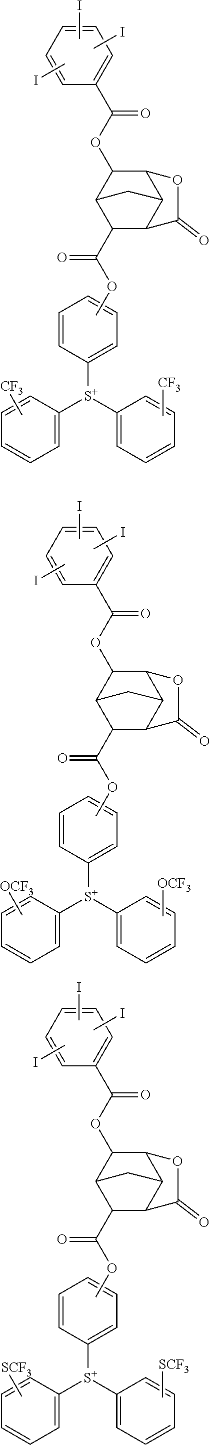

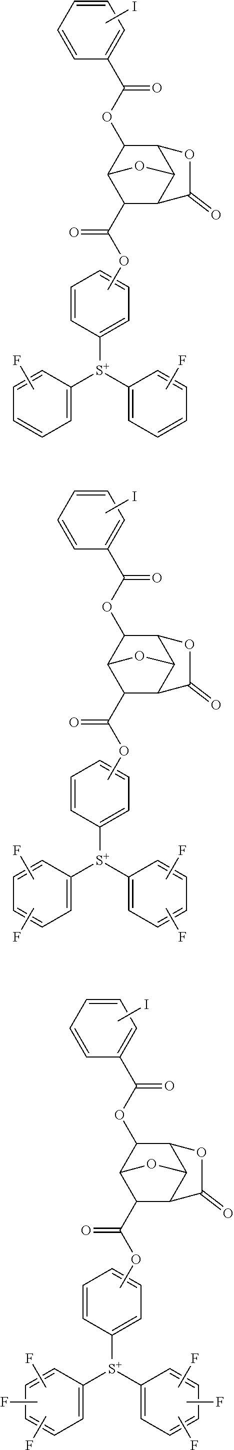

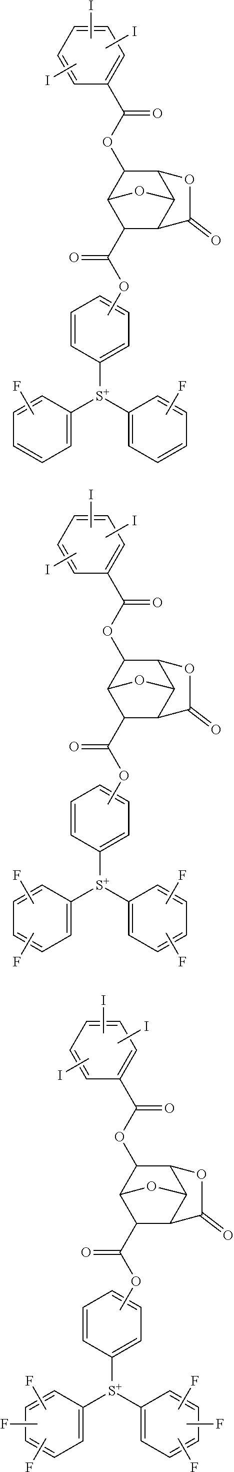

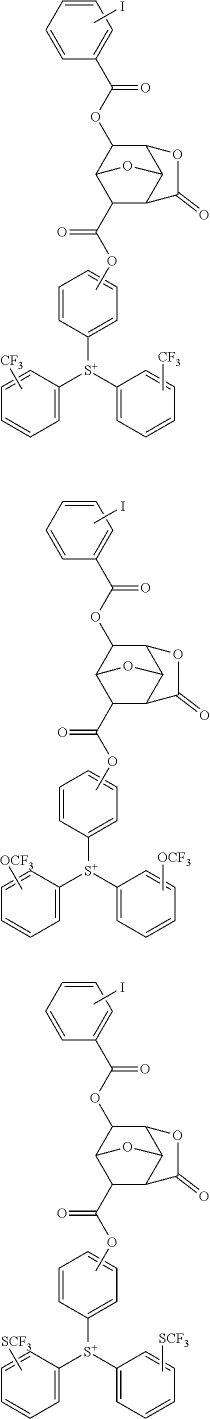

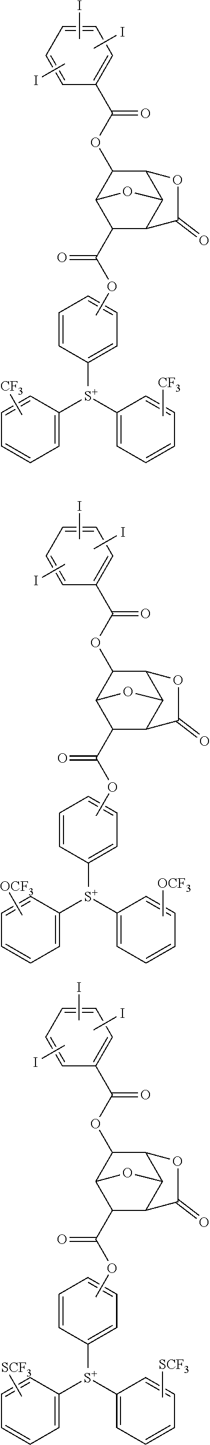

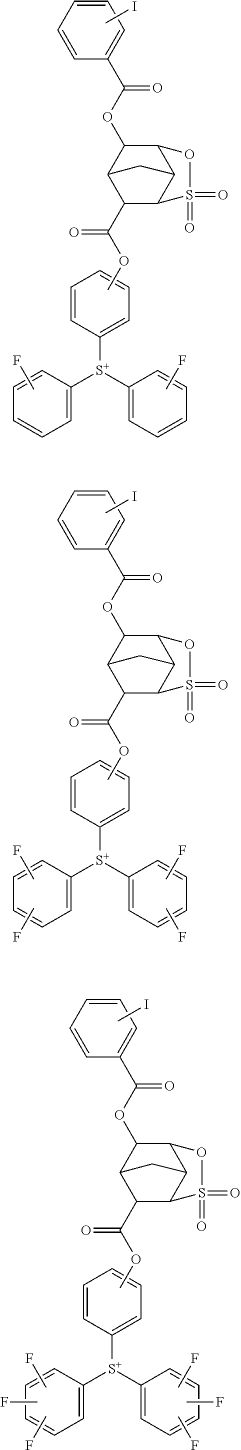

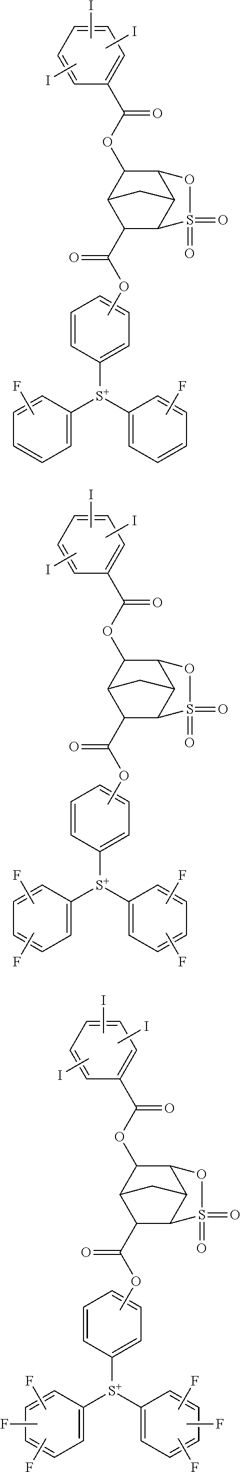

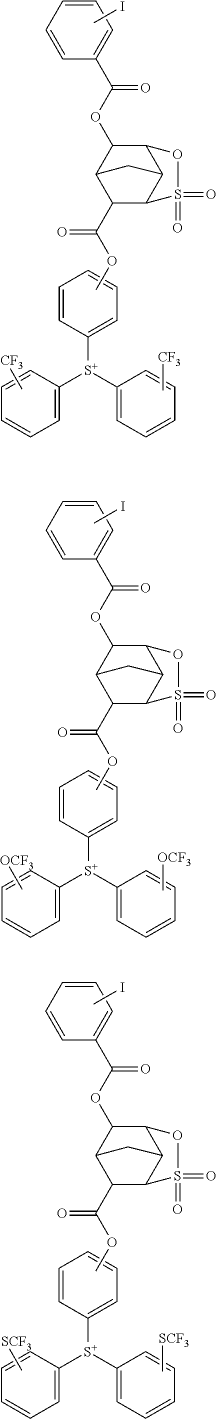

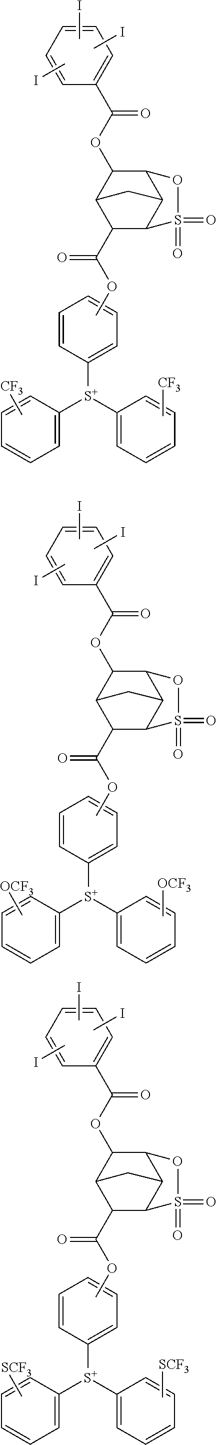

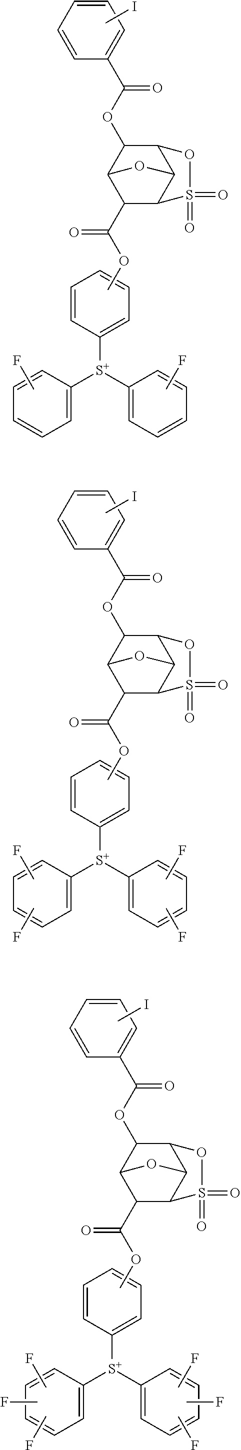

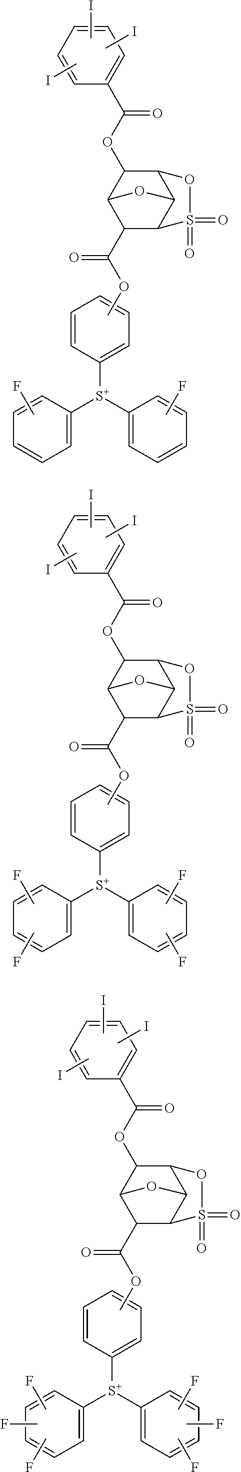

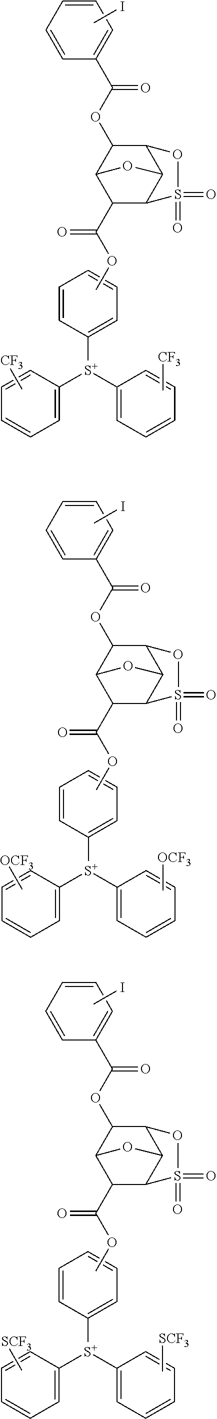

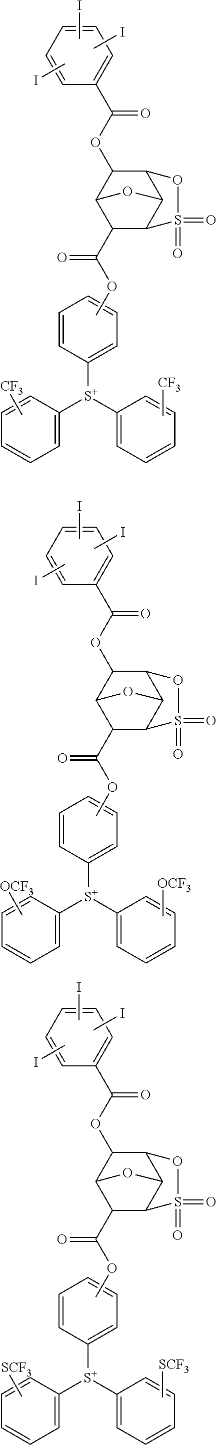

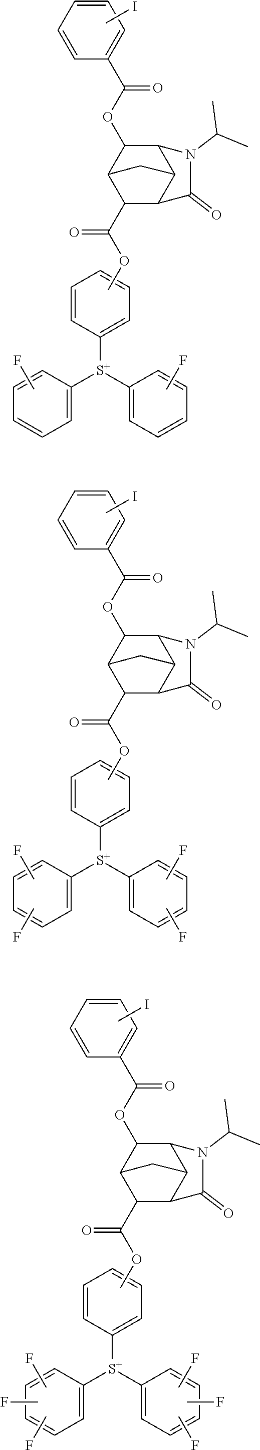

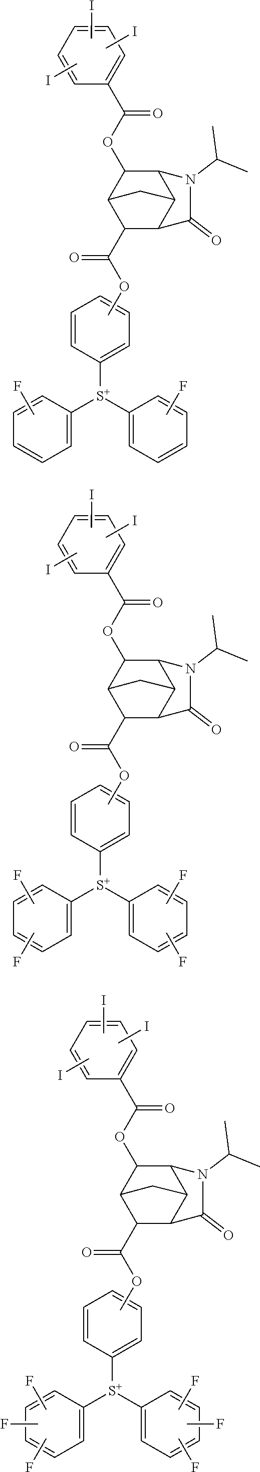

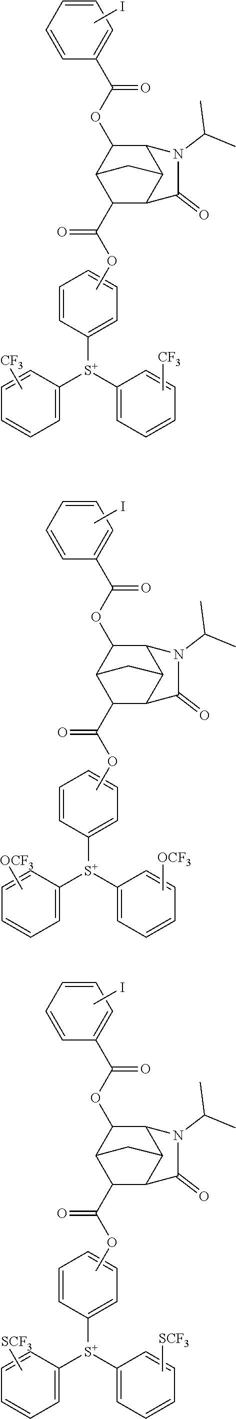

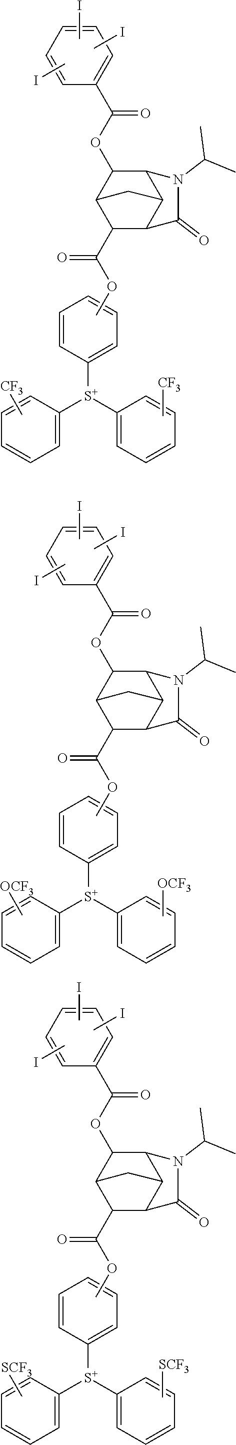

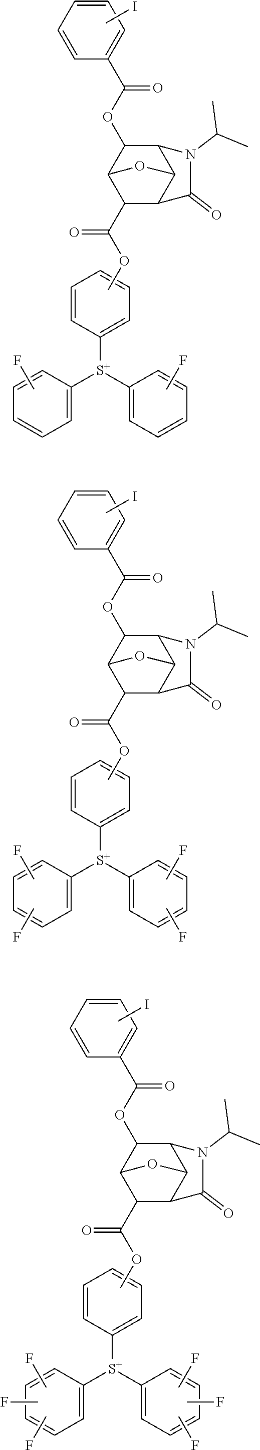

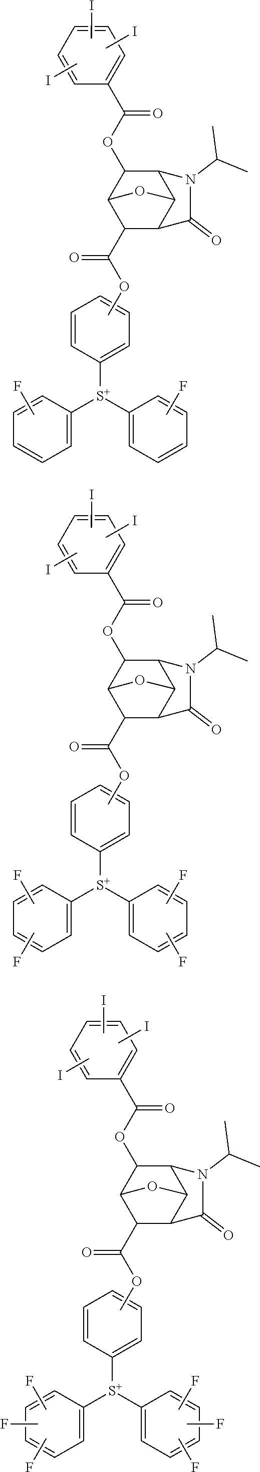

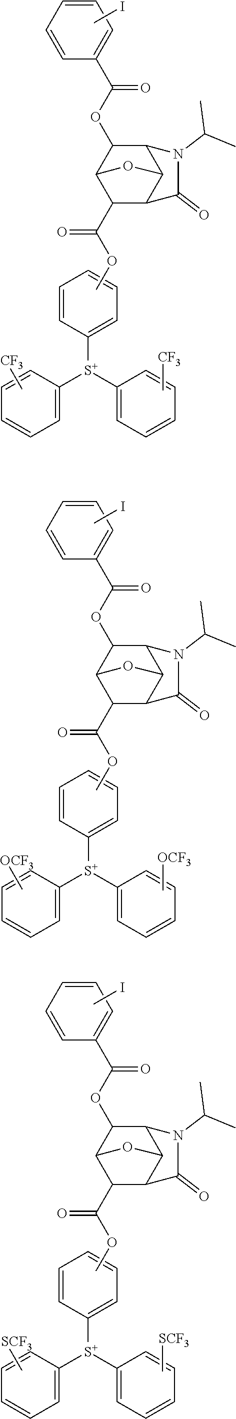

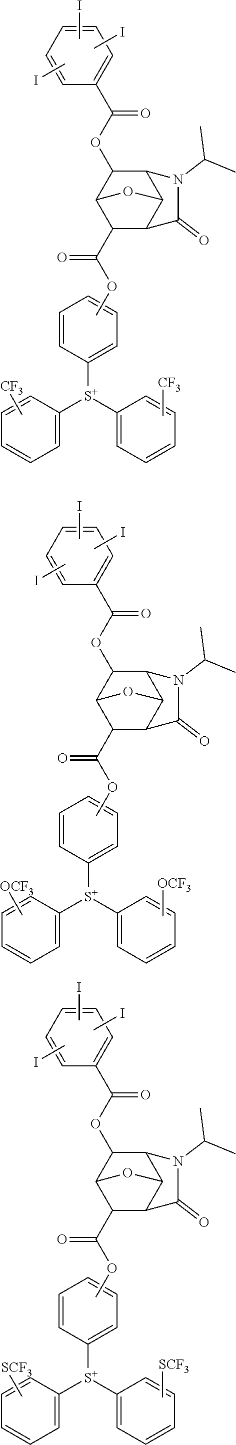

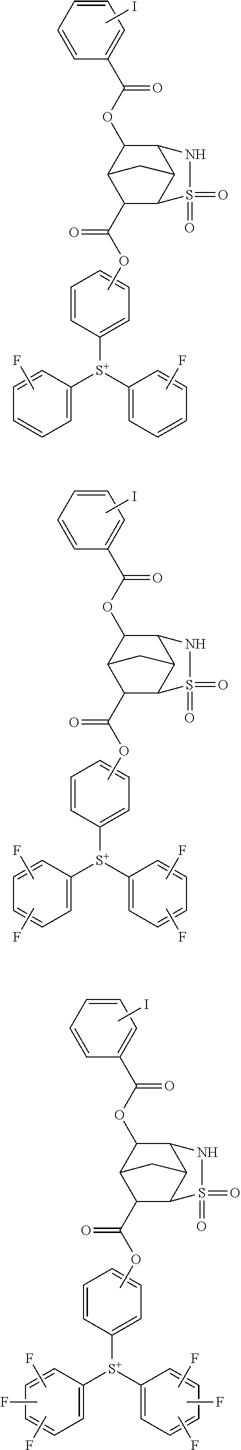

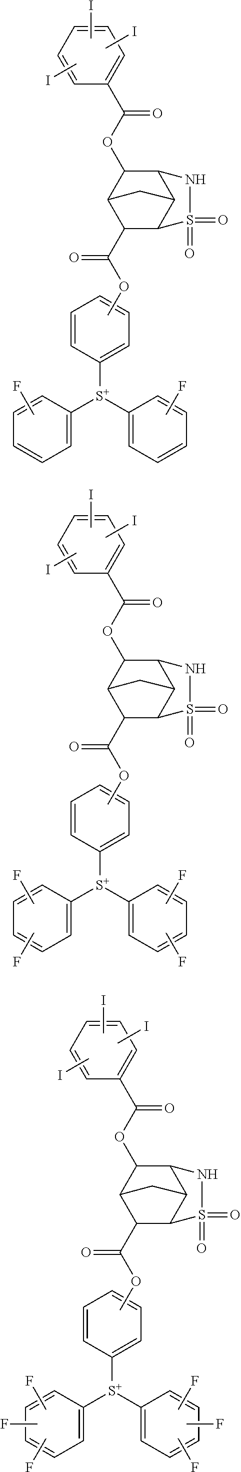

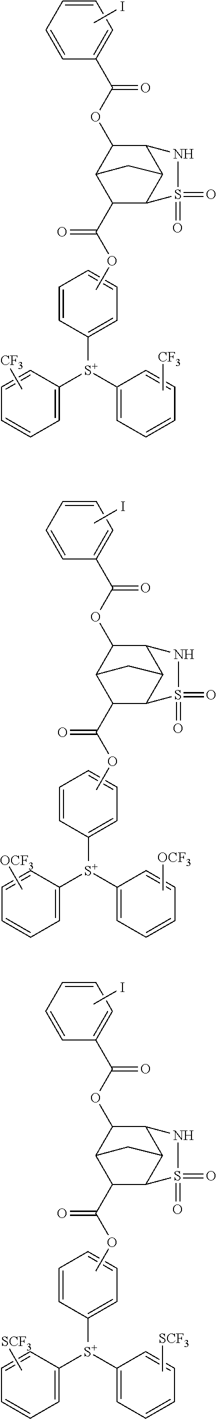

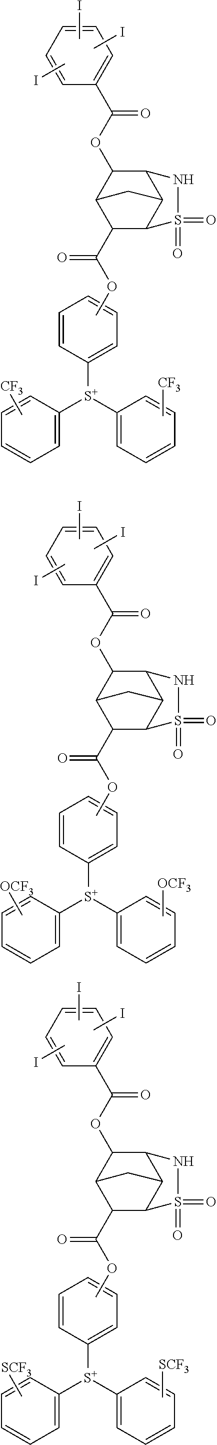

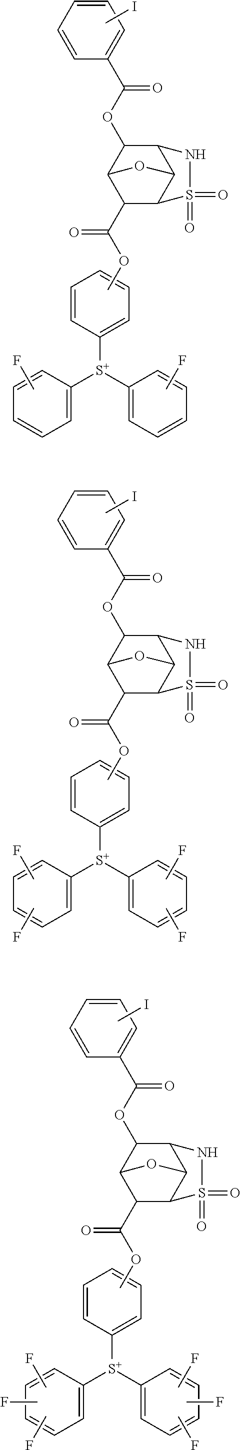

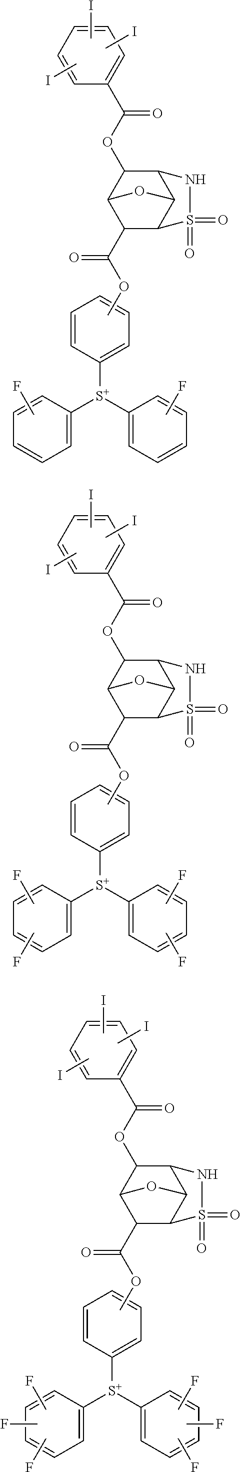

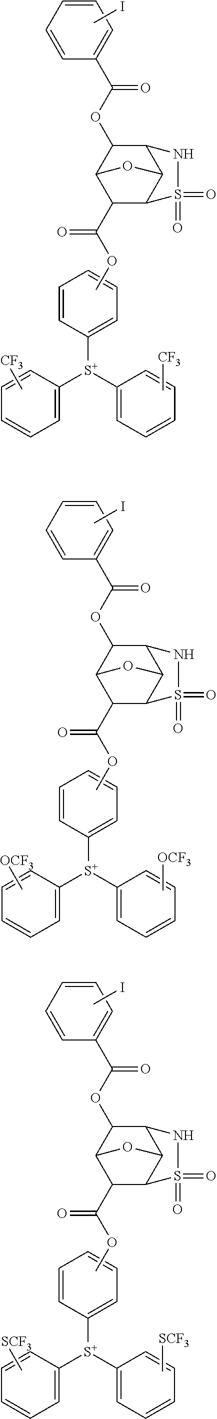

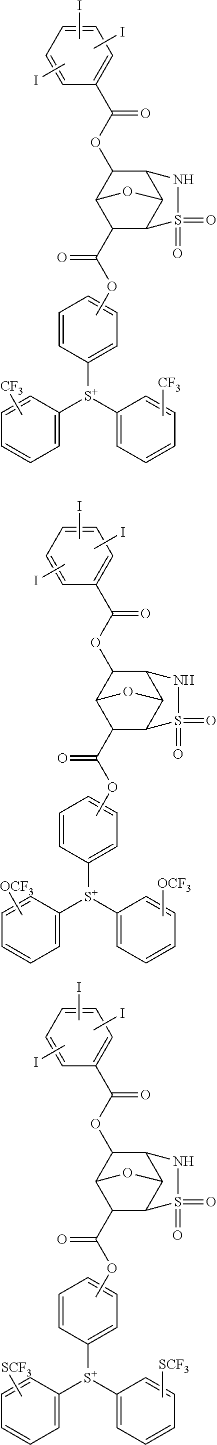

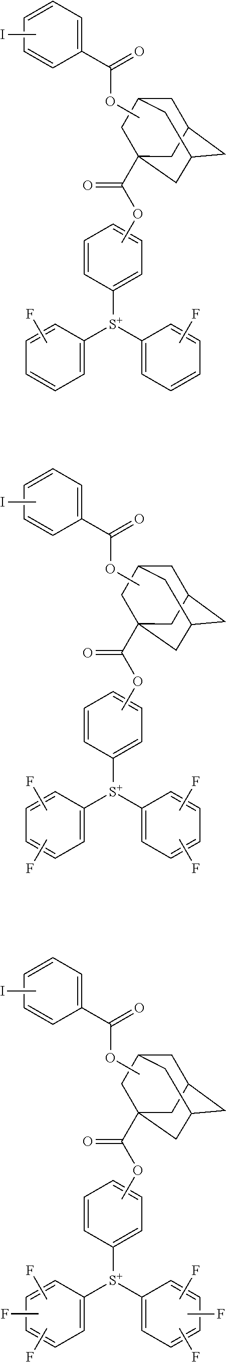

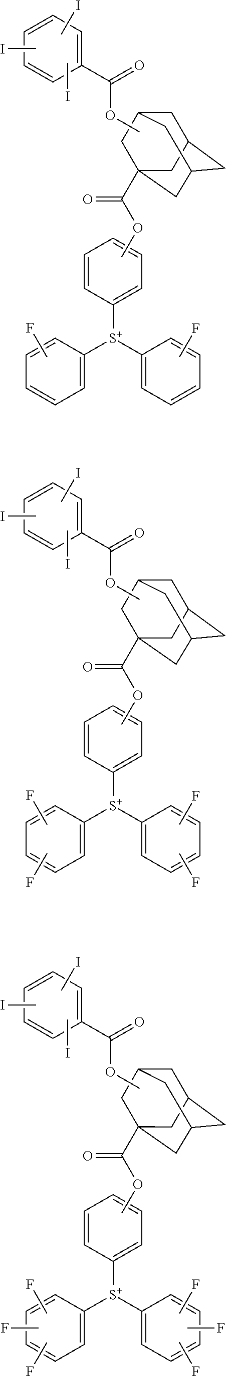

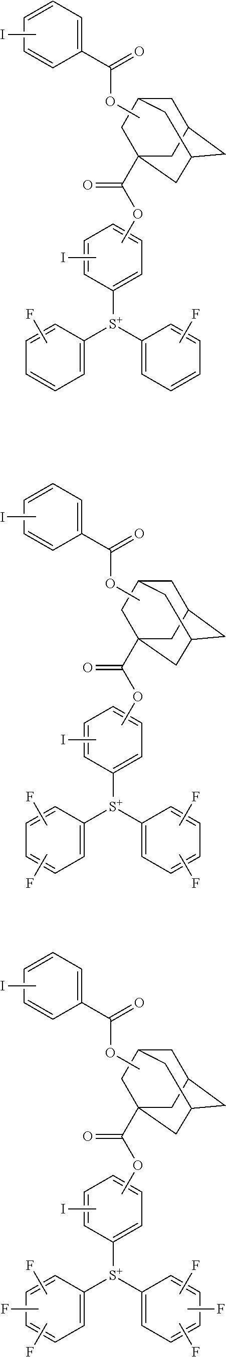

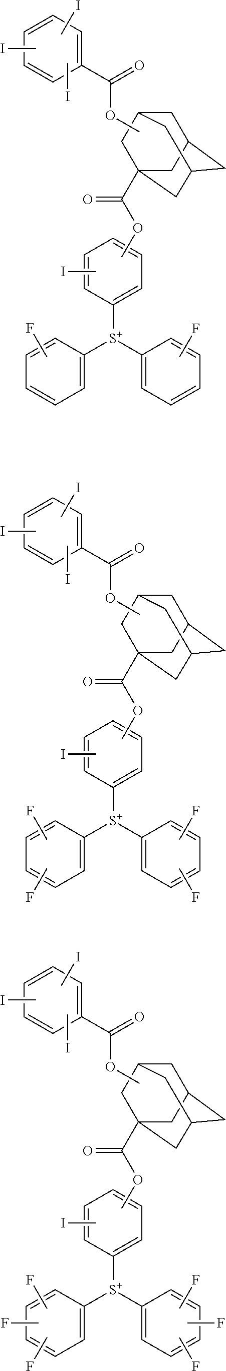

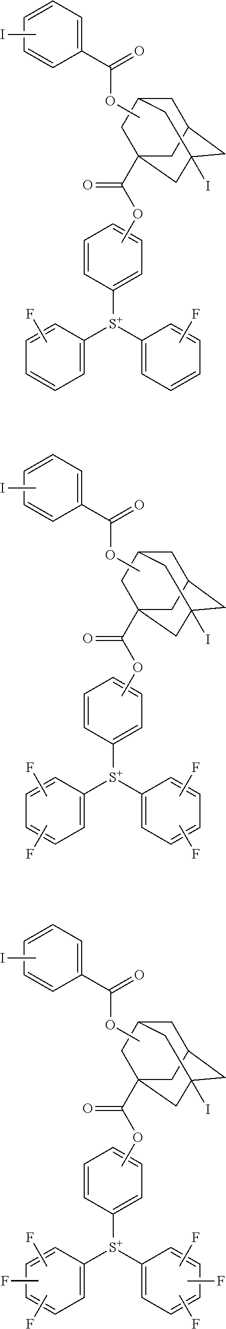

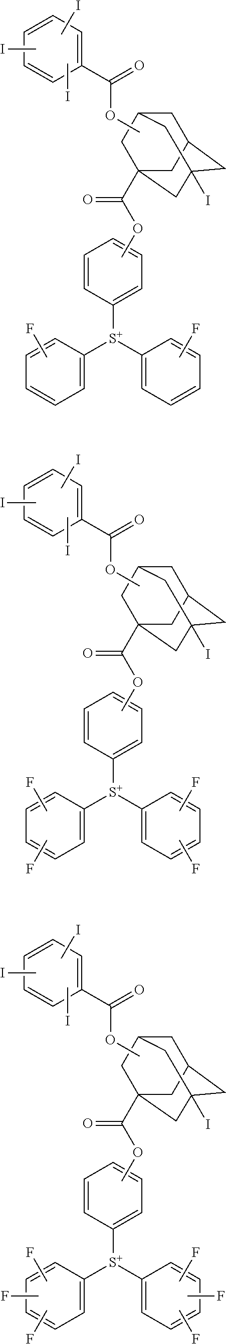

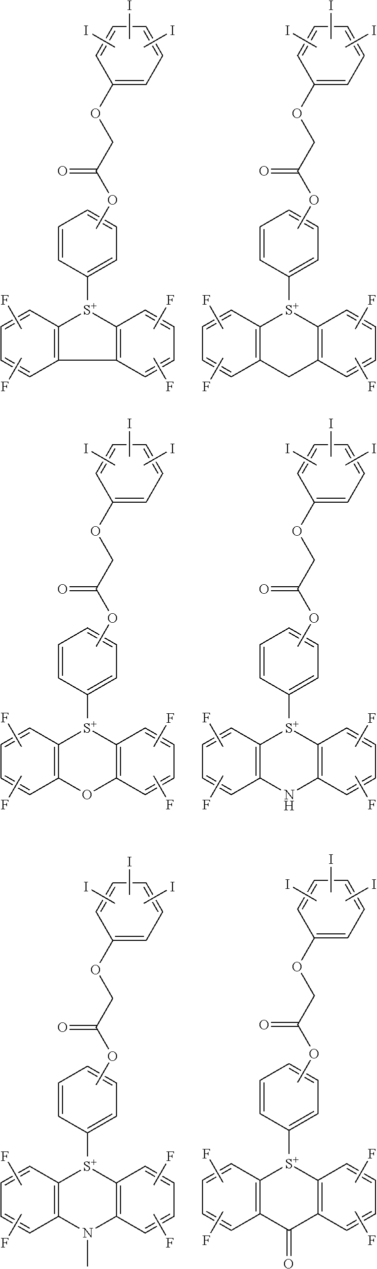

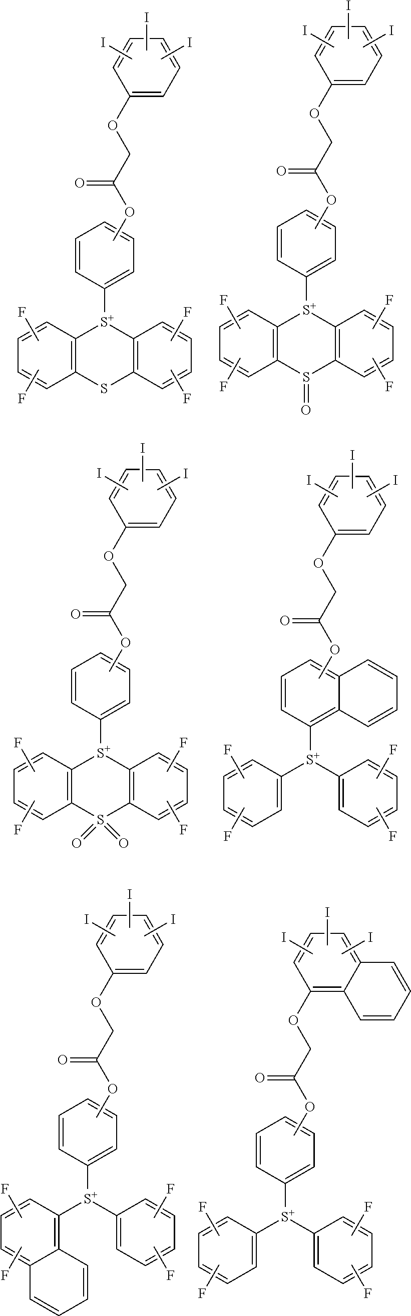

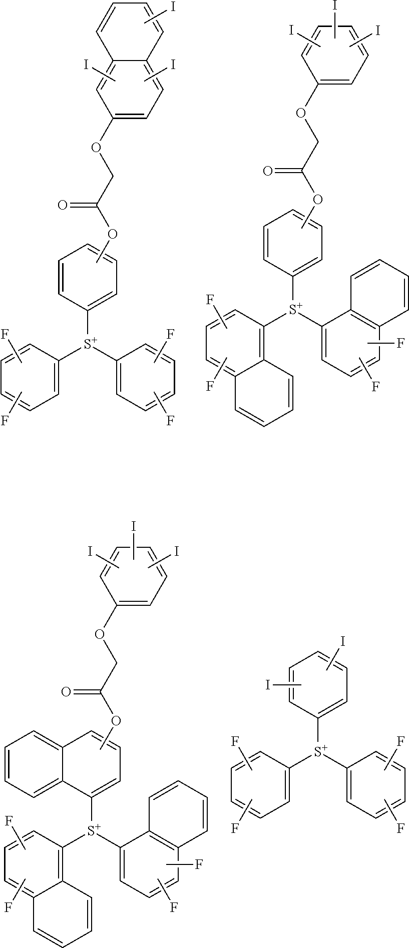

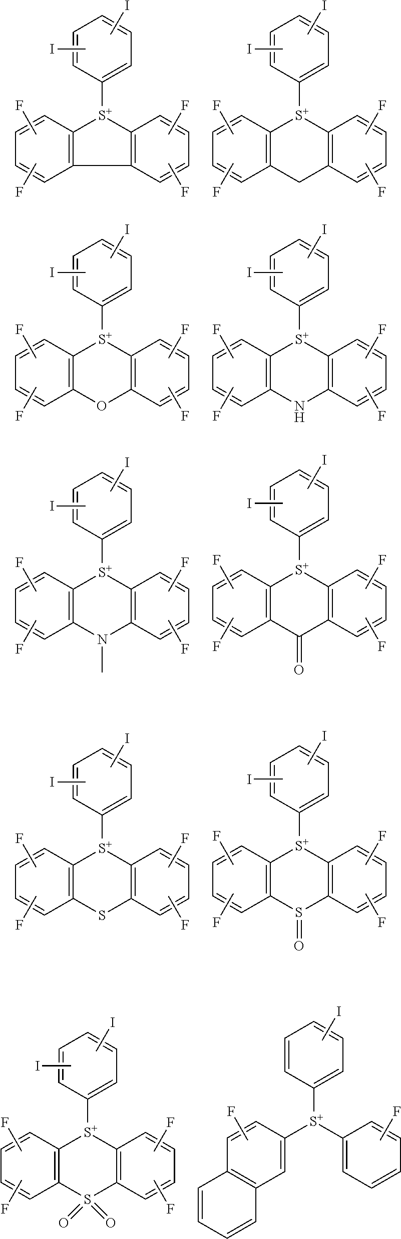

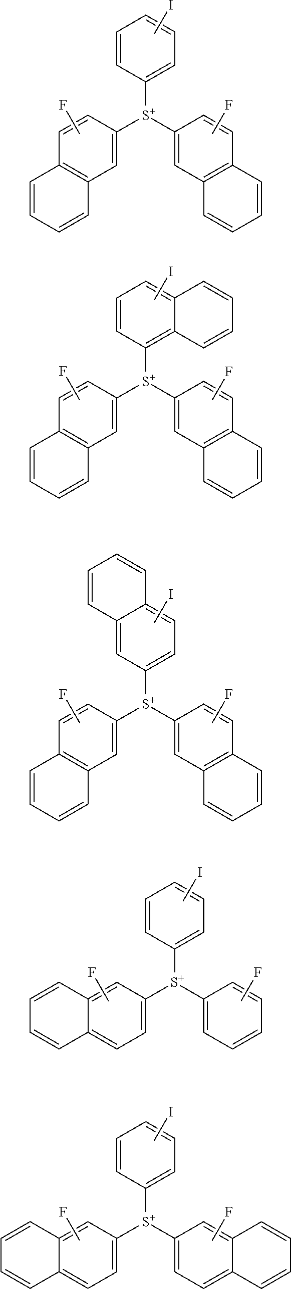

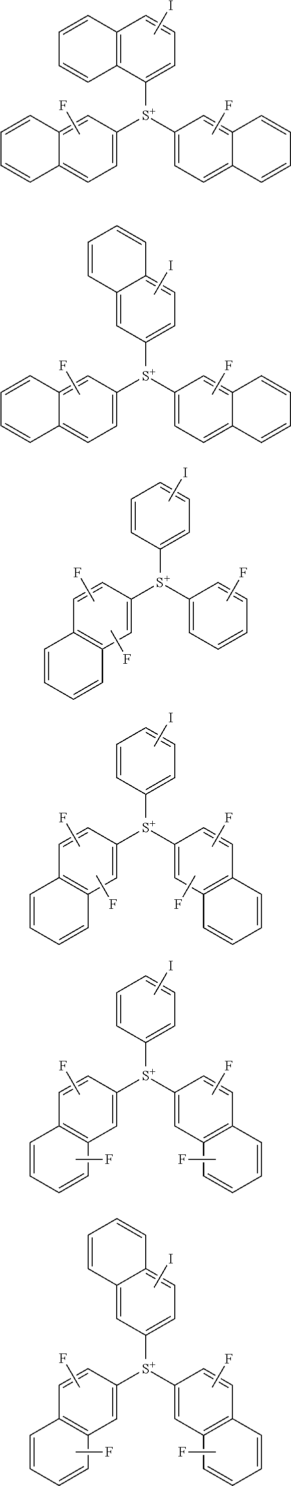

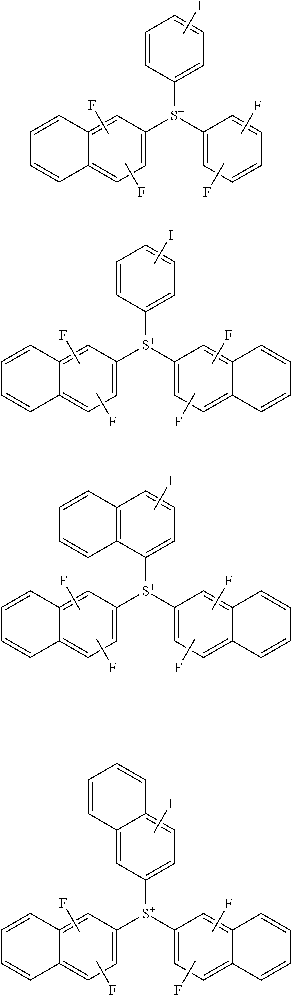

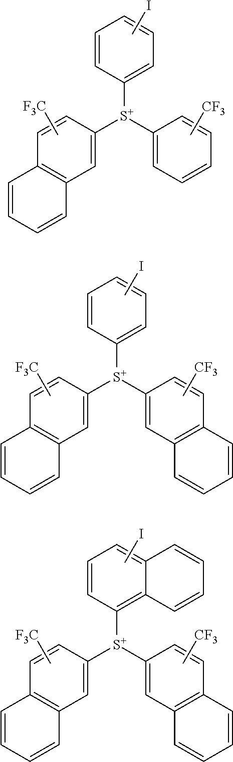

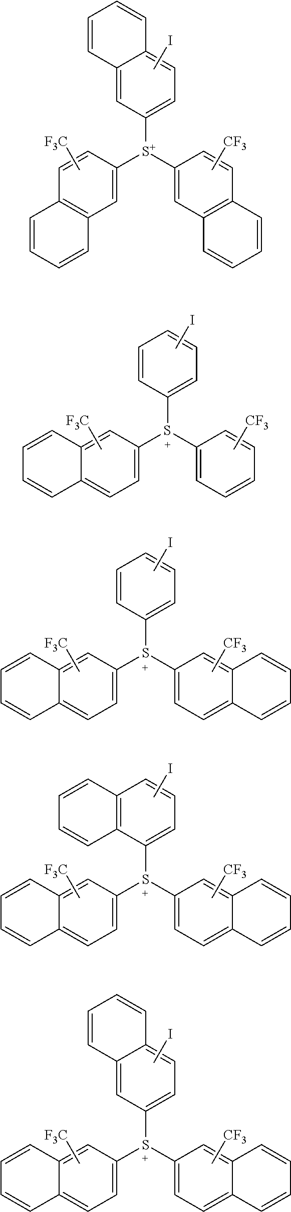

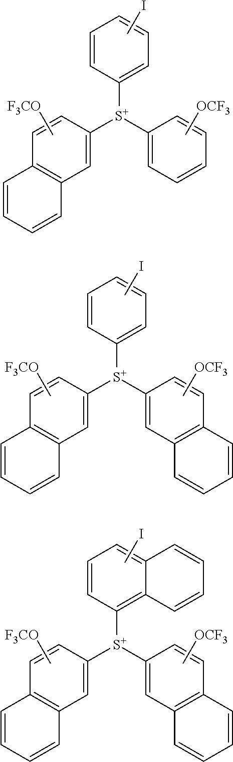

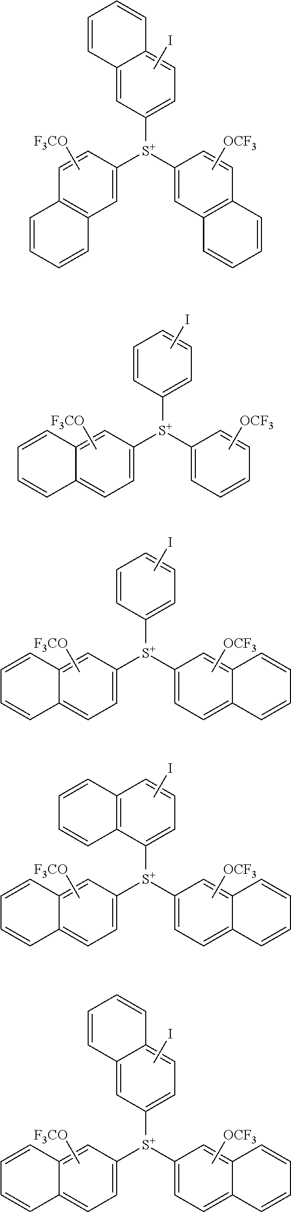

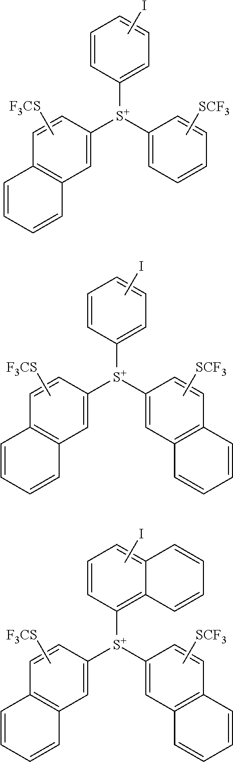

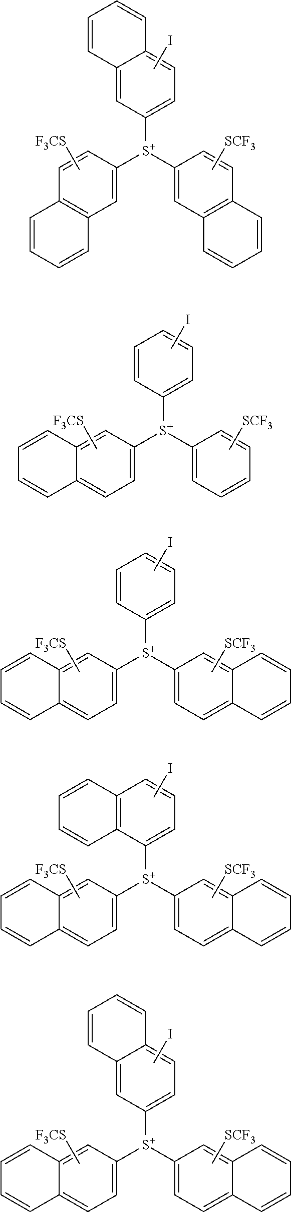

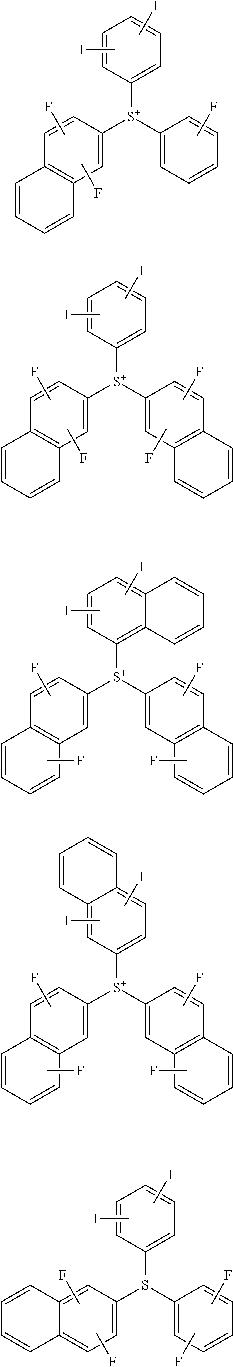

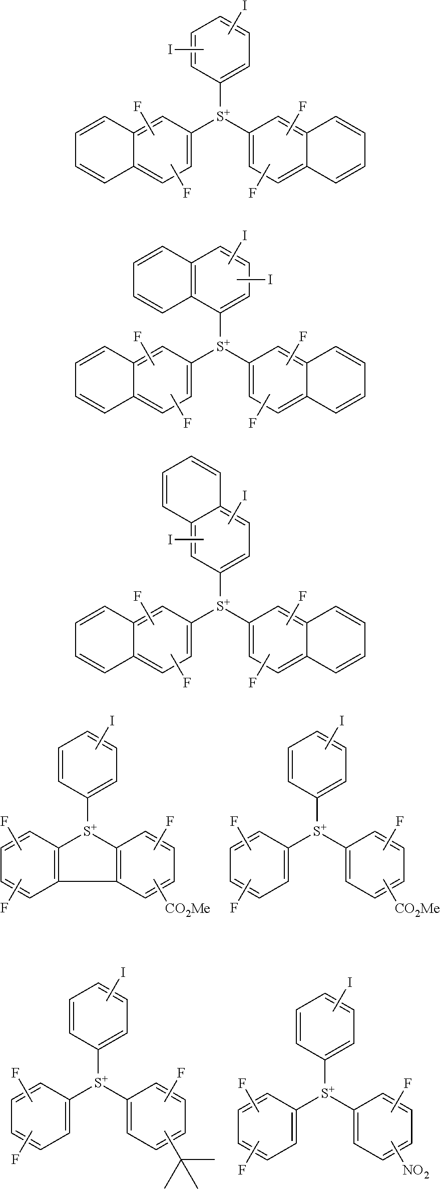

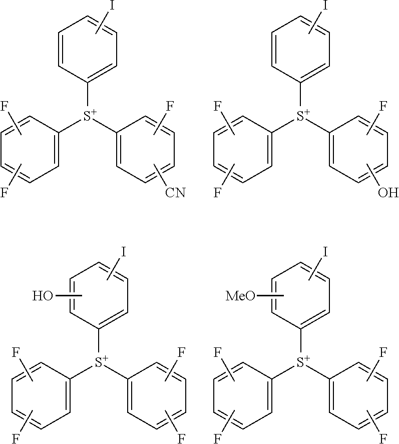

Examples of the iodonium cation include those described in JP-A 2024-000259, paragraph [0181], but are not limited thereto.

The ammonium cation preferably has the formula (am-1).

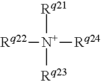

In formula (am-1), R^{q21} to R^{q24} are each independently a C₁-C₄₀ hydrocarbyl group which may contain a heteroatom. R^{q21} and R^{q22} may bond together to form a ring with the nitrogen atom to which they are attached. Examples of the hydrocarbyl group are as exemplified above for the hydrocarbyl groups R¹ to R³ in formula (A).

Examples of the ammonium cation having formula (am-1) are shown below, but are not limited thereto.

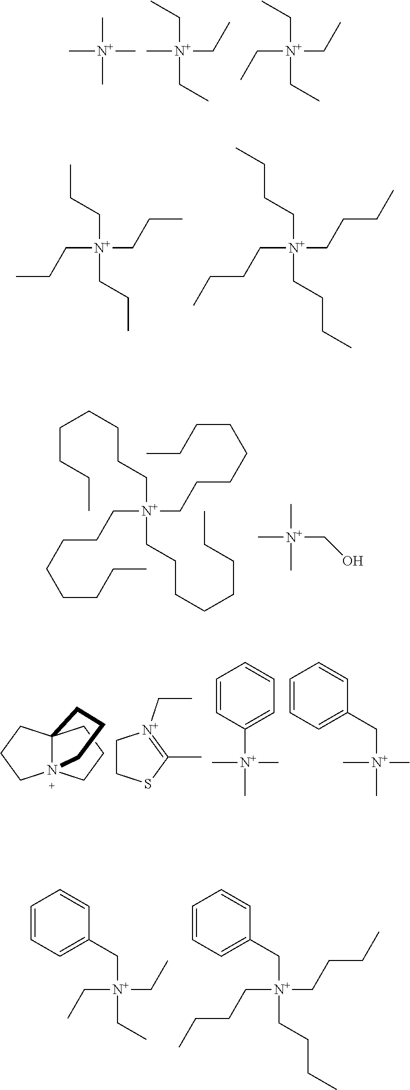

Examples of the onium salt having formula (1) or (2) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt having formula (1) or (2) functions as a quencher in the chemically amplified resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (1) or (2) functions as a quencher when used in combination with an onium salt type photoacid generator having a conjugated base of a strong acid (typically α-fluorinated sulfonic acid) as the counter anion. In a system using a mixture of an onium salt capable of generating a strong acid (typically α-fluorinated sulfonic acid) and an onium salt capable of generating a weak acid (typically non-fluorinated sulfonic acid or carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Also useful as the quencher (C) are onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755.

If a photoacid generator capable of generating a strong acid is an onium salt, an exchange from the strong acid generated upon exposure to high-energy radiation to a weak acid can take place as mentioned above, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the onium salt having formula (1) or (2) is used as the quencher (C), the amount of the onium salt used is preferably 0.1 to 20 parts by weight, more preferably 0.1 to 10 parts by weight per 80 parts by weight of the base polymer (A). As long as the amount of onium salt type quencher is in the range, a satisfactory resolution is available without a substantial lowering of sensitivity. The onium salt having formula (1) or (2) may be used alone or in admixture.

The chemically amplified resist composition may comprise a nitrogen-containing compound as the quencher (C). Suitable nitrogen-containing compounds include primary, secondary and tertiary amine compounds, specifically amine compounds having a hydroxy group, ether bond, ester bond, lactone ring, cyano group, or sulfonate ester bond, as described in JP-A 2008-111103, paragraphs [0146]-[0164], primary or secondary amine compounds protected with a carbamate group, as described in JP 3790649, and a compound having a structure in which any of a lactone ring, sultone ring, lactam ring, and a sultam ring and an acid labile group coexist, as described in JP 7615989.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the quencher (C), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (D) Photoacid Generator

The chemically amplified resist composition may comprise (D) a photoacid generator. The photoacid generator is not particularly limited as long as it is capable of generating an acid upon exposure to high-energy radiation. The preferred photoacid generators are compounds having the formulae (3) and (4).

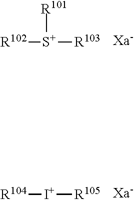

In formulae (3) and (4), R¹⁰¹ to R¹⁰⁵ are each independently halogen or a C₁-C₂₀ hydrocarbyl group which may contain a heteroatom. Any two of R¹⁰¹, R¹⁰² and R¹⁰³ may bond together to form a ring with the sulfur atom to which they are attached. Examples of the hydrocarbyl group are as exemplified above for the hydrocarbyl groups R¹ to R³ in formula (A). In the hydrocarbyl groups, some or all of the hydrogen atoms may be substituted by a moiety containing a heteroatom such as oxygen, sulfur, nitrogen, or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—), or haloalkyl moiety.

Examples of the cation in the sulfonium salt having formula (3) include, but are not limited to, those exemplified above for the cation in the sulfonium salt monomer having formula (A), those described in JP-A 2024-003744, paragraphs [0102]-[0125], WO 2024/128017, paragraphs [0044]-[0049], and JP 7491173, paragraphs [0035]-[0046], and those exemplified above for the sulfonium cation having formula (sulfo-1). Examples of the cation in the iodonium salt having formula (4) include those described in JP-A 2024-000259, paragraph [0181], but are not limited thereto.

In formulae (3) and (4), Xa⁻ is an anion of strong acid, which is typically selected from the formulae (Xa-1) to (Xa-4).

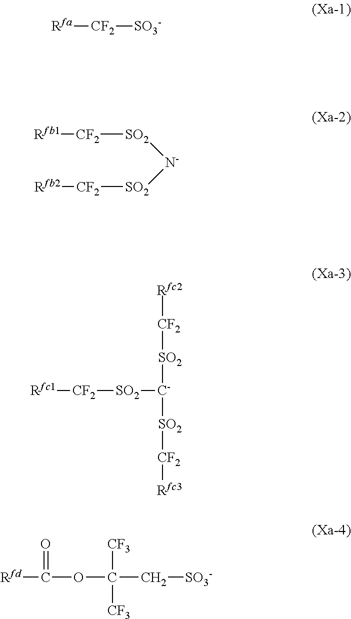

In formula (Xa-1), R^{fa} is fluorine or a C₁-C₆₀ hydrocarbyl group which may contain a heteroatom. The hydrocarbyl group may be saturated or unsaturated, and may be straight, branched or cyclic. Examples thereof are as exemplified below for hydrocarbyl group R^{fa1} in formula (Xa-1-1).

Of the anions of formula (Xa-1), a structure having the formula (Xa-1-1) is preferred.

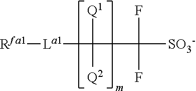

In formula (Xa-1-1), Q¹ and Q² are each independently hydrogen, fluorine or a C₁-C₆ fluorinated saturated hydrocarbyl group. It is preferred for improving solvent solubility that at least one of Q¹ and Q² be trifluoromethyl. m is 0, 1, 2, 3 or 4, particularly preferably 1. R^{fa1} is a C₁-C₄₀ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation.

In formula (Xa-1-1), the C₁-C₄₀ hydrocarbyl group R^{fa1} may be saturated or unsaturated, and may be straight, branched or cyclic. Examples thereof include C₁-C₄₀ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosyl groups; C₃-C₄₀ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl groups; C₂-C₄₀ unsaturated aliphatic hydrocarbyl groups such as 2-propenyl and 3-cyclohexenyl groups; C₆-C₄₀ aryl groups such as phenyl, 1-naphthyl, 2-naphthyl, and 9-fluorenyl groups; C₇-C₄₀ aralkyl groups such as benzyl and diphenylmethyl groups; C₇-C₄₀ aromatic ring-containing polycyclic hydrocarbyl groups such as 9,10-ethano-9,10-dihydroanthryl, and 6,13-ethano-6,13-dihydropentacenyl groups; C₁₇-C₄₀ hydrocarbyl groups having a steroid skeleton; and combinations thereof.

In the hydrocarbyl groups, some or all of the hydrogen atoms may be substituted by a moiety containing a heteroatom such as oxygen, sulfur, nitrogen, or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—), or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

In formula (Xa-1-1), L^{a1} is a single bond, ether bond, ester bond, sulfonate ester bond, carbonate bond or carbamate bond. From the aspect of synthesis, L^{a1} is preferably an ether bond or ester bond, more preferably ester bond.

Examples of the anion having formula (Xa-1) are shown below, but are not limited thereto. Herein, Q¹ is as defined above, and Ac is acetyl.

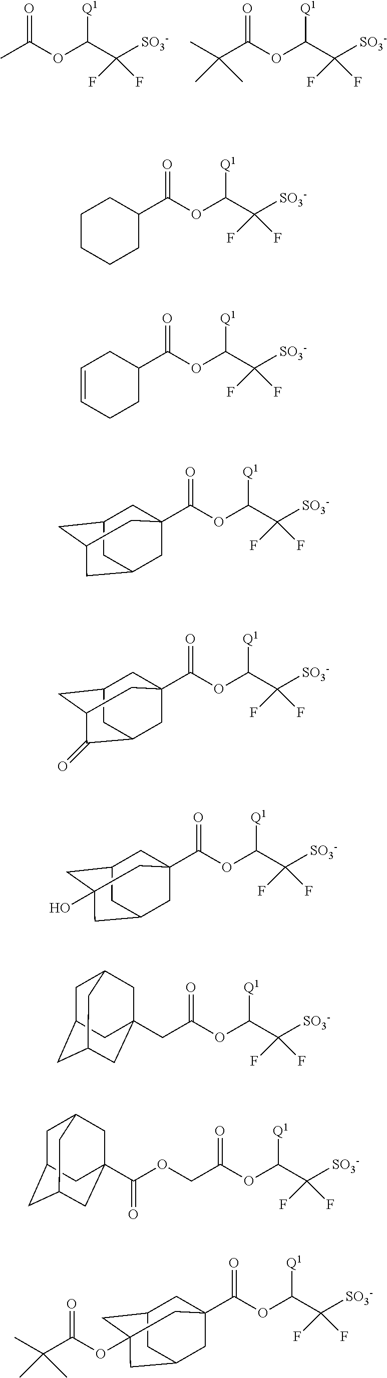

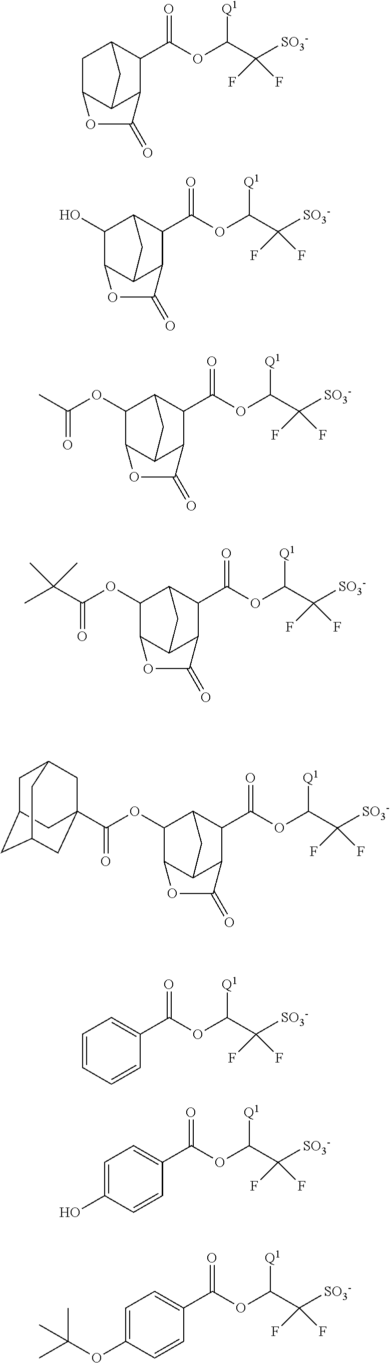

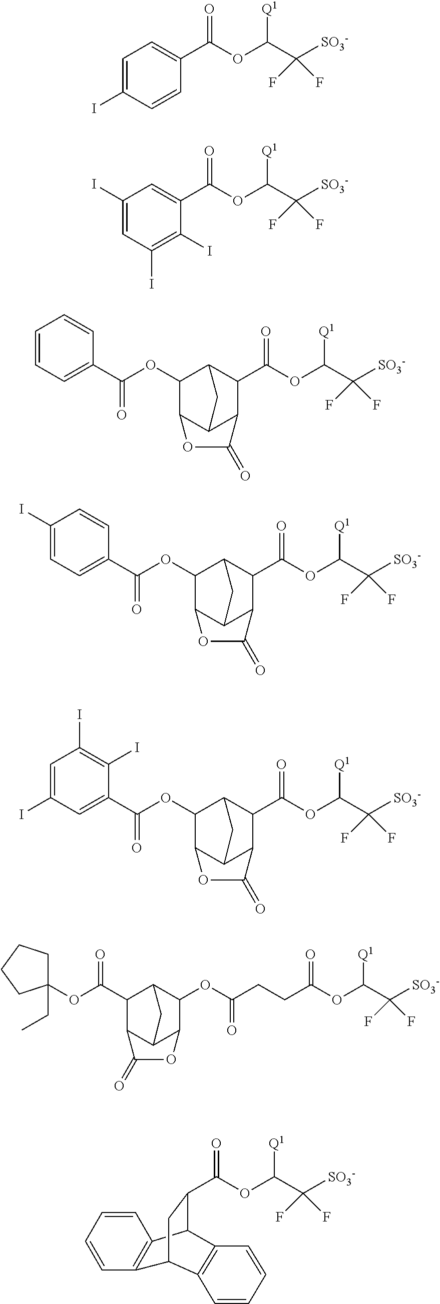

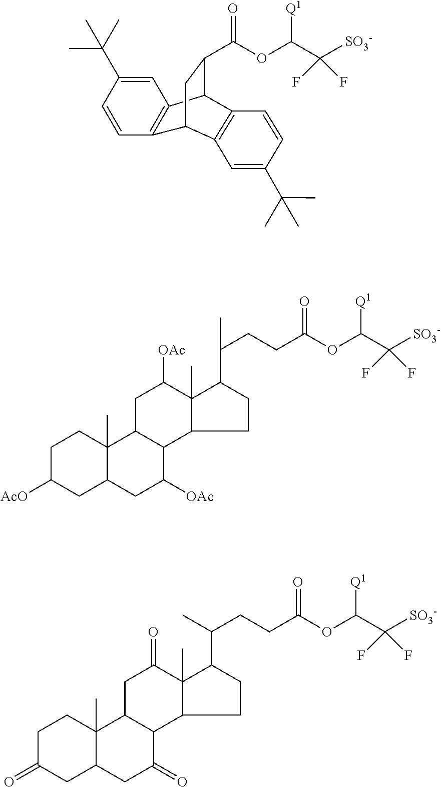

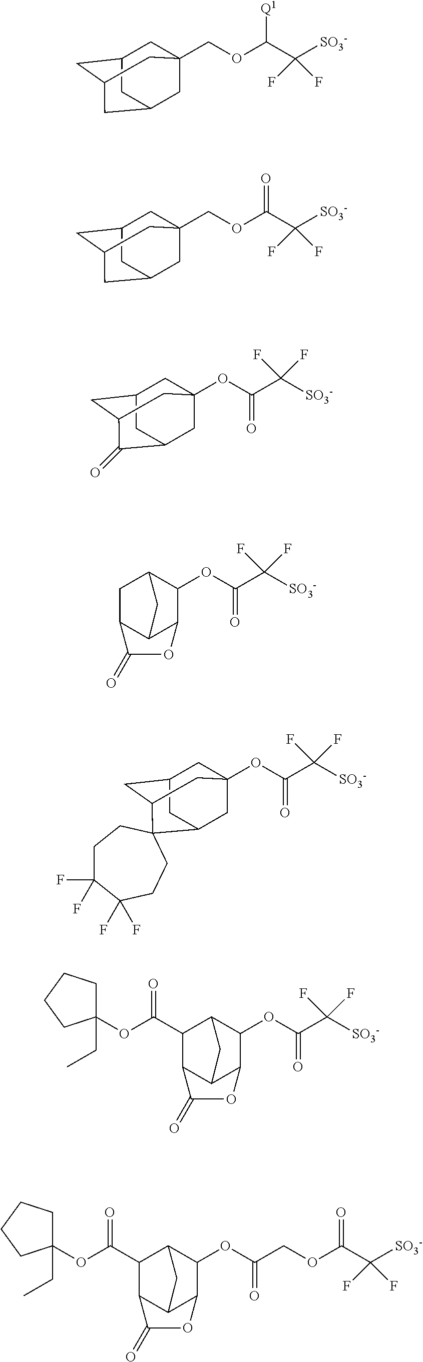

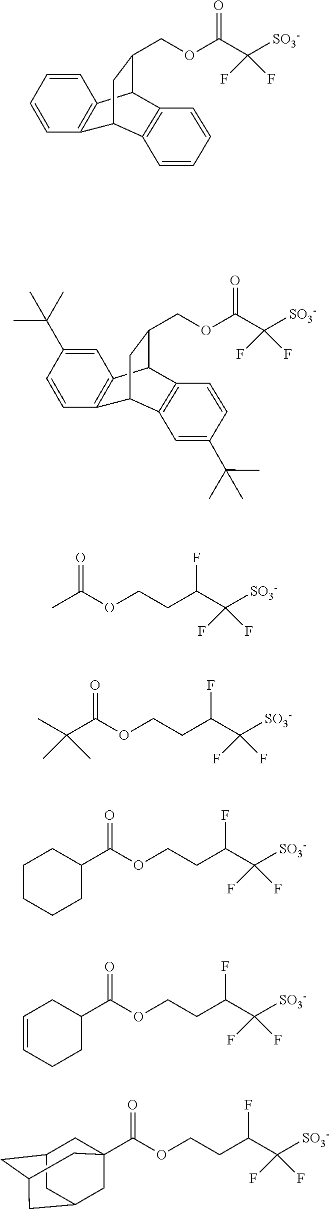

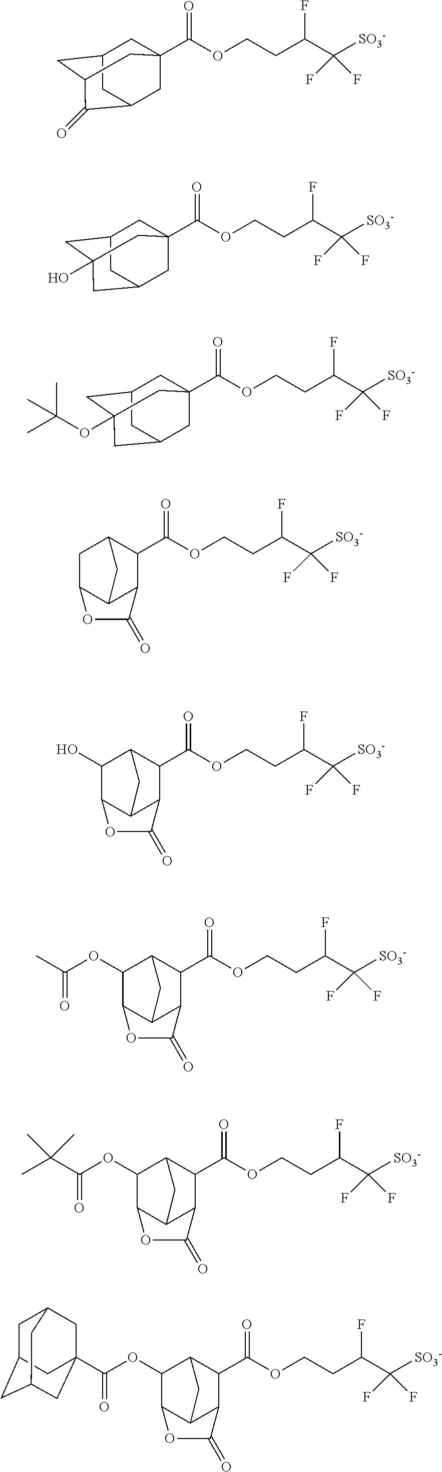

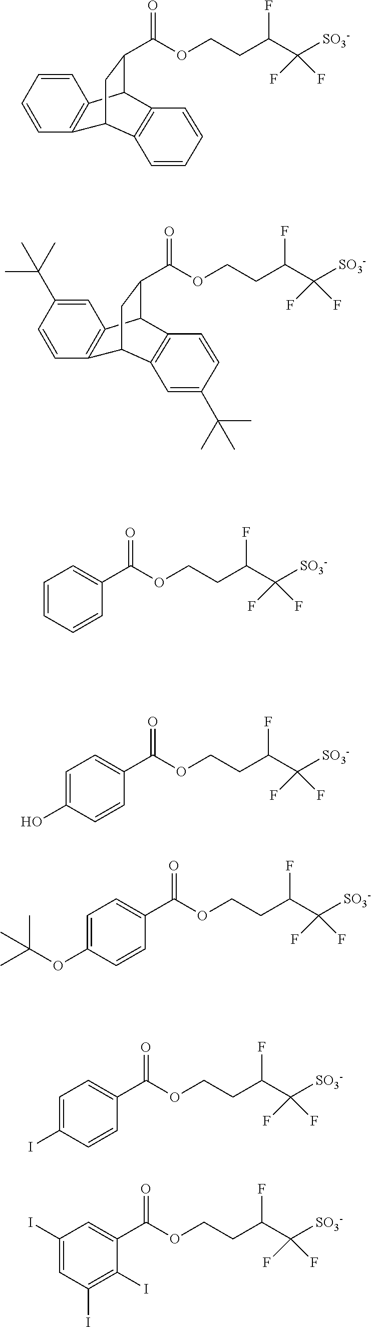

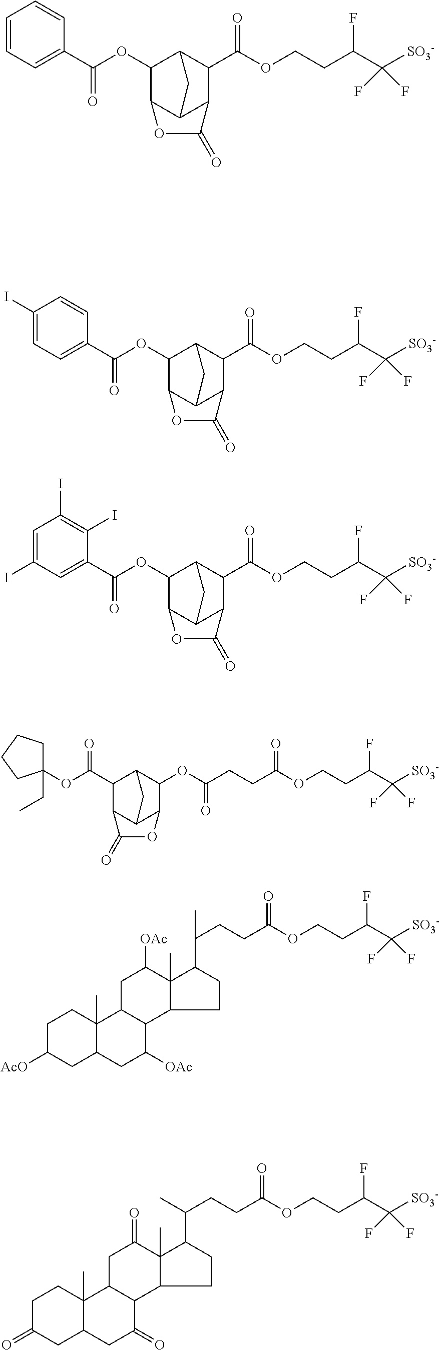

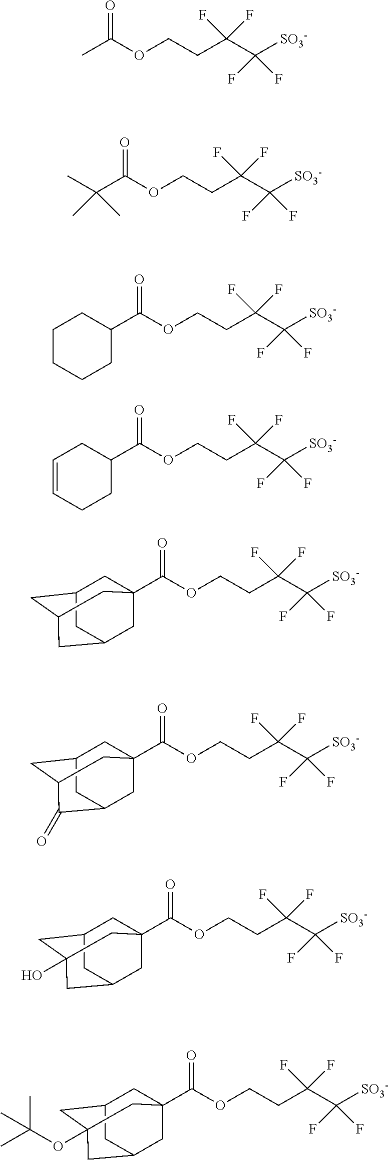

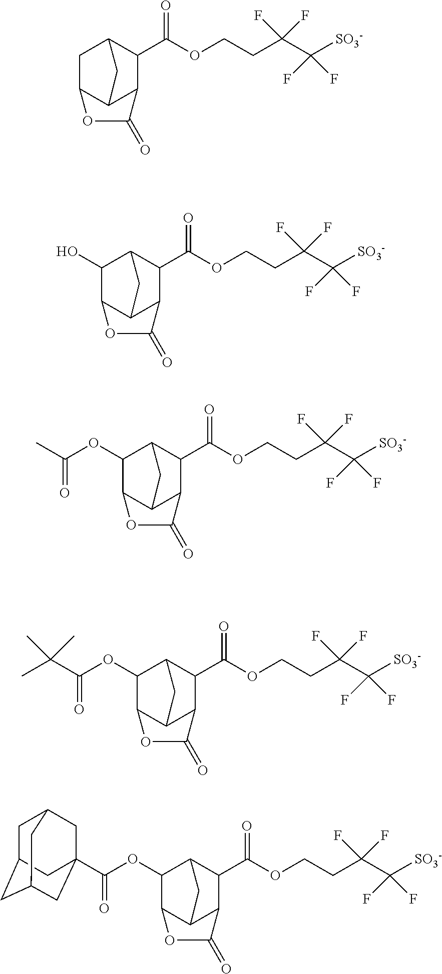

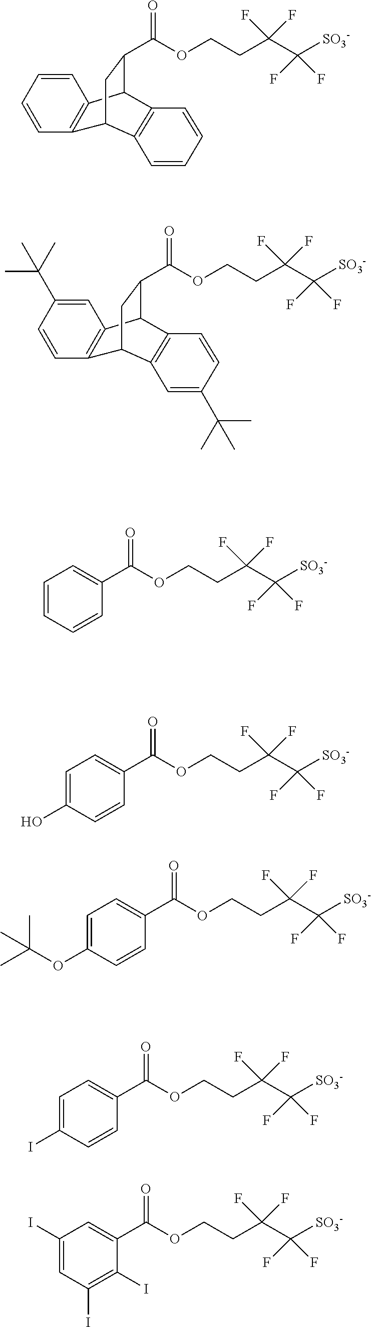

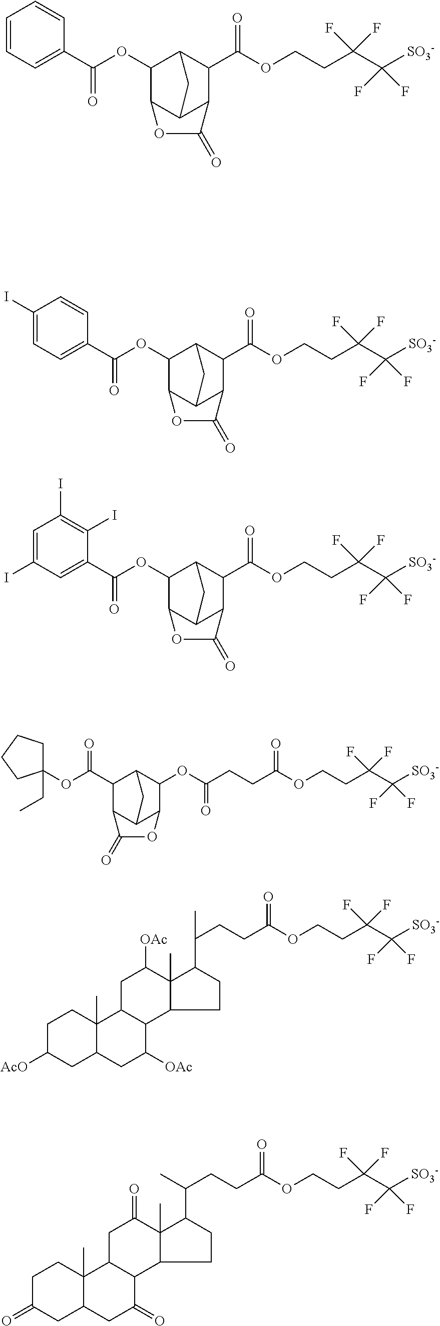

Other examples of the anion having formula (Xa-1) are as described in JP-A 2025-023601, paragraphs [0042]-[0049], JP-A 2021-070692, paragraphs [0015]-[0017], JP 7091762, paragraphs [0145] and [0148], JP 7628783, paragraphs [0022]-[0023], and JP 7109178, paragraph [0224].

In formula (Xa-2), R^{fb1} and R^{fb2} are each independently fluorine or a C₁-C₄₀ hydrocarbyl group which may contain a heteroatom. The hydrocarbyl group may be saturated or unsaturated, and may be straight, branched or cyclic. Examples thereof are as exemplified above for the hydrocarbyl group R^{fa1} in formula (Xa-1-1). R^{fb1} and R^{fb2} are preferably fluorine or a C₁-C₄ straight fluorinated alkyl group. A pair of R^{fb1} and R^{fb2} may bond together to form a ring with the linkage (—CF₂—SO₂—N⁻—SO₂—CF₂—) to which they are attached, and the R^{fb1}—R^{fb2} group is preferably a fluorinated ethylene or fluorinated propylene group.

In formula (Xa-3), R^{fc1}, R^{fc2} and R^{fc3} are each independently fluorine or a C₁-C₄₀ hydrocarbyl group which may contain a heteroatom. The hydrocarbyl group may be saturated or unsaturated, and may be straight, branched or cyclic. Examples thereof are as exemplified above for the hydrocarbyl group R^{fa1} in formula (Xa-1-1). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the R^{fc1}—R^{fc2} group is preferably a fluorinated ethylene or fluorinated propylene group.

In formula (Xa-4), R^{fd} is a C₁-C₄₀ hydrocarbyl group which may contain a heteroatom. The hydrocarbyl group may be saturated or unsaturated, and may be straight, branched or cyclic. Examples thereof are as exemplified above for the hydrocarbyl group R^{fa1} in formula (Xa-1-1). In the hydrocarbyl groups, some or all of the hydrogen atoms may be substituted by a moiety containing a heteroatom such as oxygen, sulfur, nitrogen, or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—), or haloalkyl moiety.

Examples of the anion having formula (Xa-4) are shown below, but are not limited thereto.

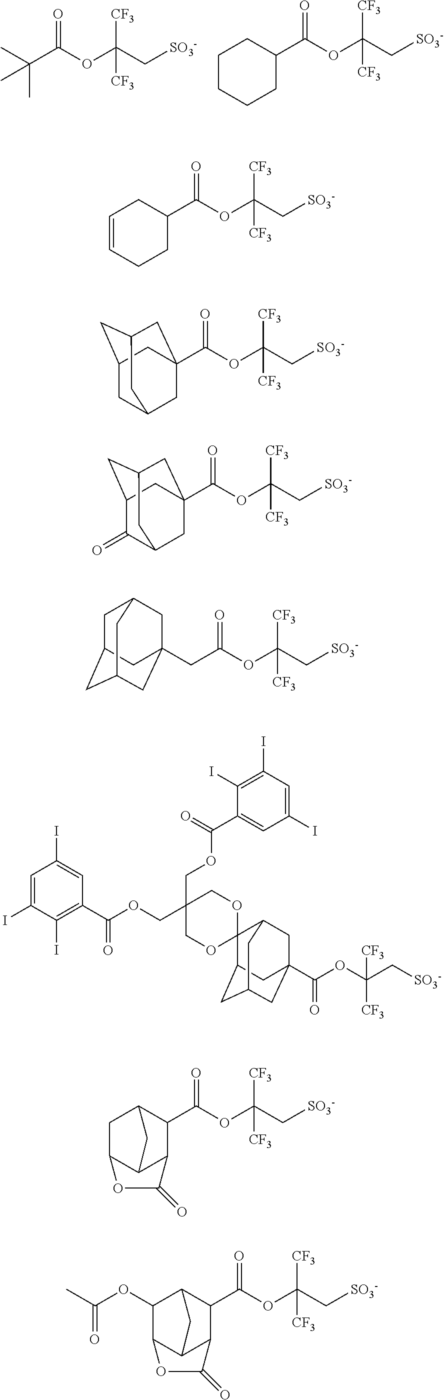

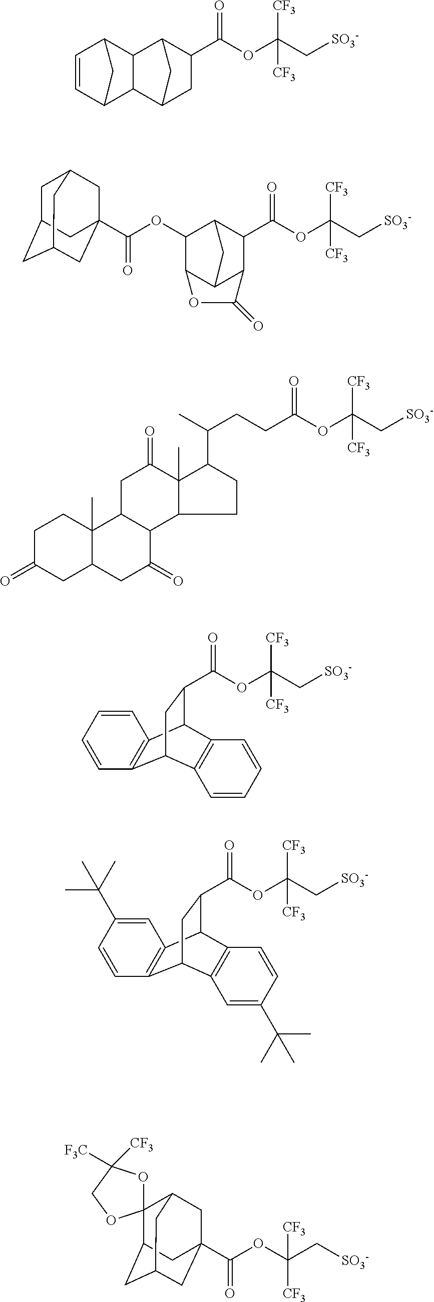

Other examples of the anion having formula (Xa-4) are as described in JP-A 2025-023601, in paragraphs [0050]-[0058].

Examples of the anion Xa⁻ include anions having an aromatic ring substituted with iodine or bromine. Examples of the anion include anions of the formula (Xa-5).

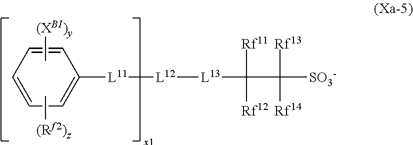

In formula (Xa-5), x is 1, 2 or 3. y is 1, 2, 3, 4 or 5.
z is 0, 1, 2 or 3. Provided that 1≤y+z≤5. y is preferably 1, 2 or 3, more preferably 2 or 3. z is preferably 0, 1 or 2.

In formula (Xa-5), X^{B1} is iodine or bromine. A plurality of X^{B1} may be identical or different when x and/or y is 2 or more.

In formula (Xa-5), L¹¹ is a single bond, ether bond, ester bond, sulfonate ester bond, carbonate bond, carbamate bond, or a C₁-C₆ saturated hydrocarbylene group, and some constituent —CH₂— of the hydrocarbylene group may be replaced by an ether bond or ester bond. The saturated hydrocarbylene group may be straight, branched or cyclic.

In formula (Xa-5), L¹² is a single bond or C₁-C₂₀ hydrocarbylene group which may contain a heteroatom when x=1, and a C₁-C₂₀ (x+1)-valent hydrocarbon group which may contain a heteroatom when x is 2 or 3.

The C₁-C₂₀ hydrocarbylene group L¹² may be saturated or unsaturated, and may be straight, branched or cyclic. Examples thereof include C₁-C₂₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl and dodecane-1,12-diyl groups; C₃-C₂₀ cyclic saturated hydrocarbylene groups such as cyclopentanediyl, cyclohexanediyl, norbornanediyl, adamantanediyl and tricyclo[5.2.1.0^{2,6}]decanediyl groups; C₂-C₂₀ unsaturated aliphatic hydrocarbylene groups such as vinylene and propene-1,3-diyl groups; C₆-C₂₀ arylene groups such as phenylene, naphthylene and anthracenediyl groups; C₇-C₂₀ aromatic ring-containing polycyclic hydrocarbylene groups such as 9,10-ethano-9,10-dihydroanthracenediyl and 6,13-ethano-6,13-dihydropentacene groups; and combinations thereof. The C₁-C₂₀ (x+1)-valent hydrocarbon group of L¹² may be saturated or unsaturated, and may be straight, branched or cyclic. Examples thereof include those exemplified above for the C₁-C₂₀ hydrocarbylene group, with one or two hydrogen atoms being eliminated.

Some or all of hydrogen atoms in the hydrocarbylene group and (x+1)-valent hydrocarbon group may be replaced by a group containing a heteroatom such as oxygen, sulfur, nitrogen, or halogen, some constituent —CH₂— in the hydrocarbylene group and (x+1)-valent hydrocarbon group may be replaced by a group containing a heteroatom such as oxygen, sulfur or nitrogen, and as a result, the hydrocarbylene group and (x+1)-valent hydrocarbon group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—), or haloalkyl moiety.

In formula (Xa-5), L¹³ is a single bond, ether bond, ester bond, sulfonate ester bond, carbonate bond or carbamate bond.

In formula (Xa-5), R^{fc} is hydroxy, carboxy, fluorine, chlorine, bromine, amino group, C₁-C₂₀ hydrocarbyl, C₁-C₂₀ hydrocarbyloxy, C₁-C₂₀ hydrocarbylthio, C₂-C₂₀ hydrocarbylcarbonyl, C₂-C₂₀ hydrocarbyloxycarbonyl, C₂-C₂₀ hydrocarbylcarbonyloxy, C₁-C₂₀ hydrocarbylsulfonyloxy, —N(R^{feA})(R^{feB}), —N(R^{feC})—C(═O)—R^{feD},

or —N(R^{feC})—C(═O)—O—R^{feD}, and the hydrocarbyl, hydrocarbyloxy, hydrocarbylthio, hydrocarbylcarbonyl, hydrocarbyloxycarbonyl, hydrocarbylcarbonyloxy, and hydrocarbylsulfonyloxy groups may contain at least one selected from fluorine, chlorine, bromine, iodine, hydroxy, amino, ester bond, and ether bond. R^{feA} and R^{feB} are each independently hydrogen or a C₁-C₆ saturated hydrocarbyl group. R^{feC} is hydrogen, or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy group. R^{feD} is a C₁-C₁₆ aliphatic hydrocarbyl group, C₆-C₁₂ aryl group or C₇-C₁₅ aralkyl group, which may contain halogen, hydroxy, a C₁-C₆ saturated hydrocarbyloxy, C₂-C₆ saturated hydrocarbylcarbonyl or C₂-C₆ saturated hydrocarbylcarbonyloxy group. The aliphatic hydrocarbyl group may be saturated or unsaturated, and may be straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In formula (Xa-5), Rf¹¹ to Rf¹⁴ are each independently hydrogen, fluorine, or trifluoromethyl, and at least one of Rf¹¹ to Rf¹⁴ is fluorine or trifluoromethyl. Rf¹¹ and Rf¹² taken together, may form a carbonyl group. More preferably, both Rf¹³ and Rf¹⁴ are fluorine.

Examples of the anion having formula (Xa-5) are shown below, but are not limited thereto. Herein, X^{B1} is as defined above.

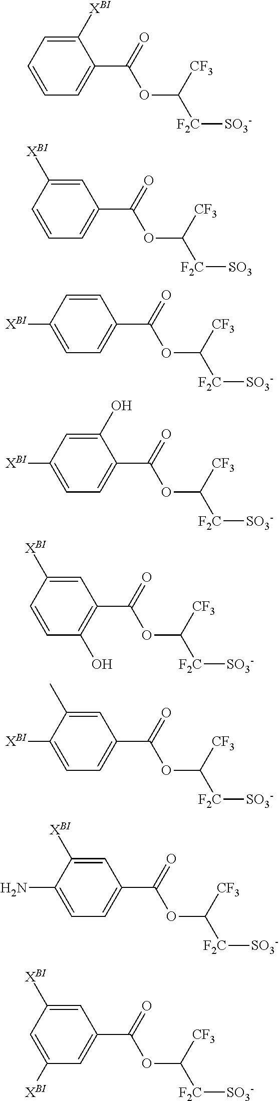

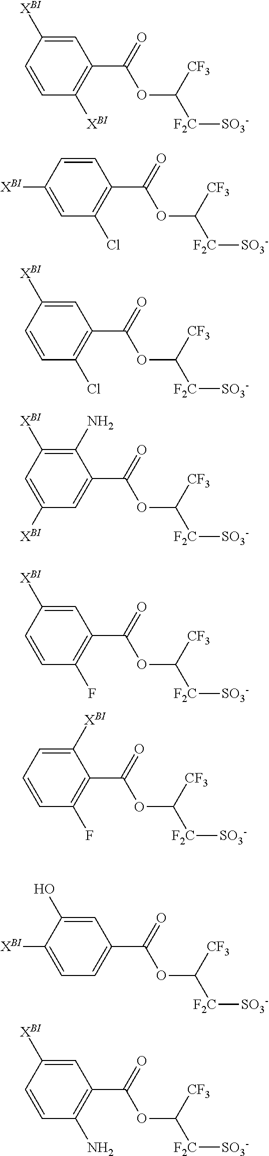

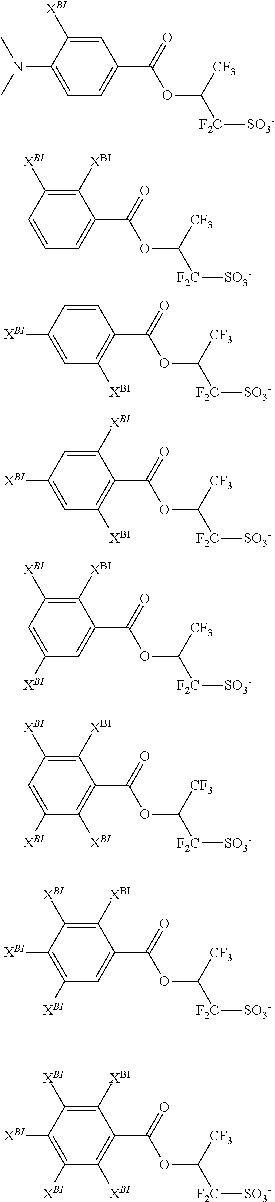

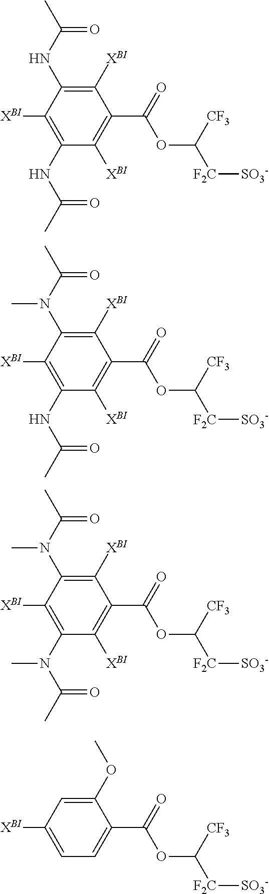

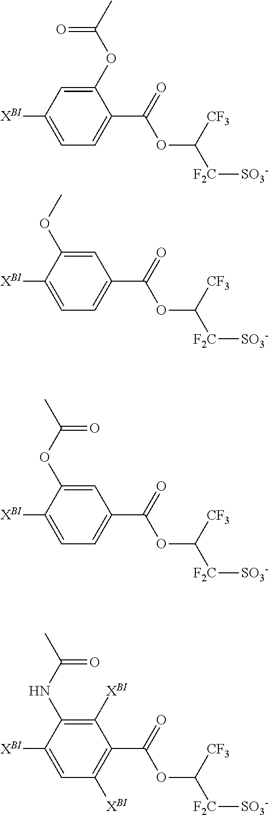

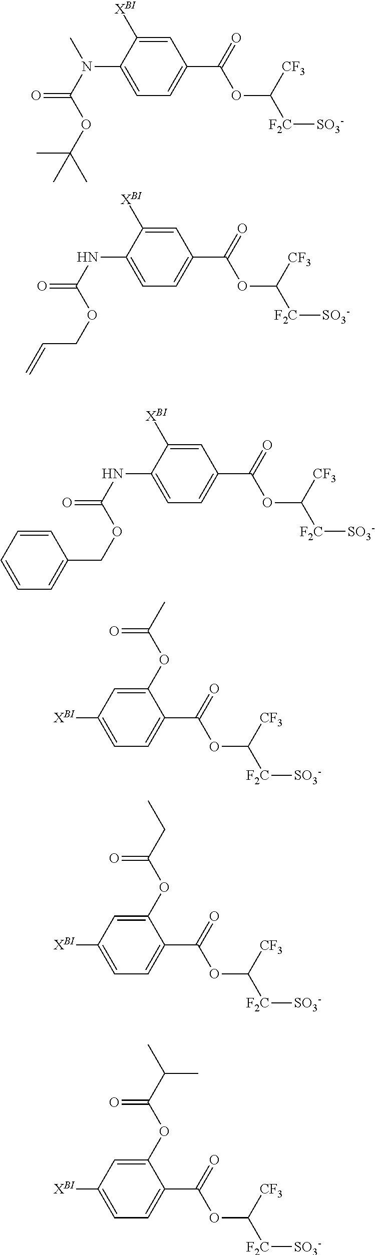

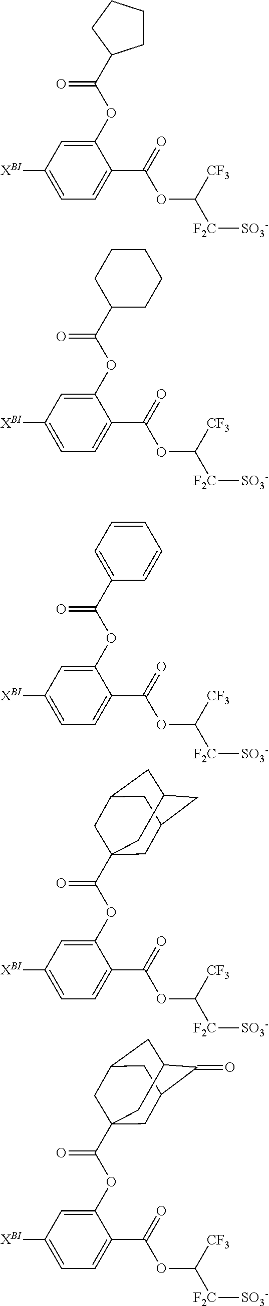

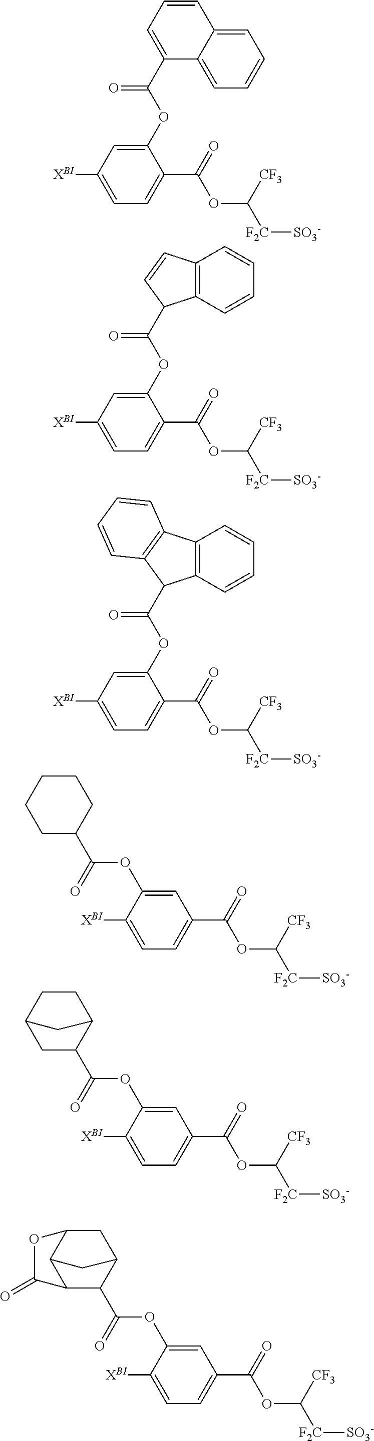

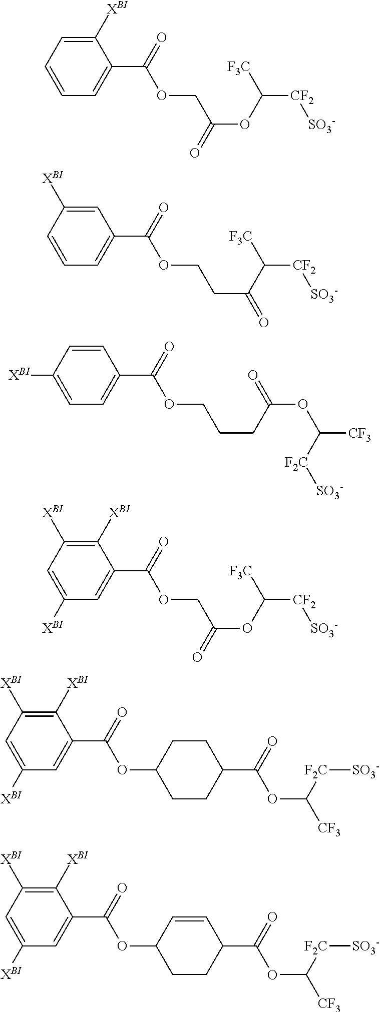

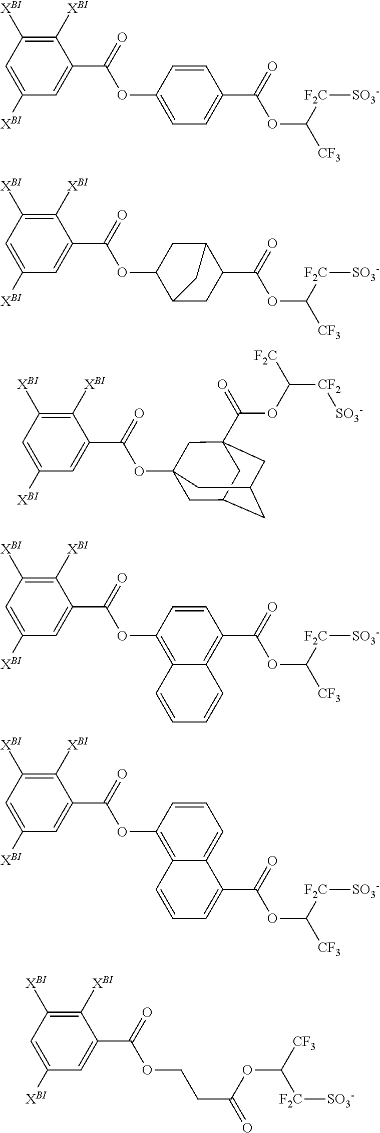

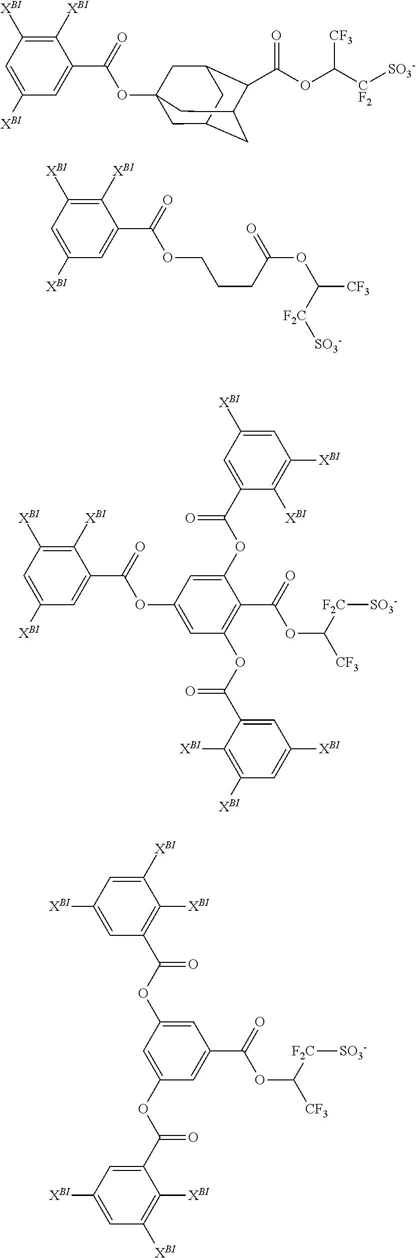

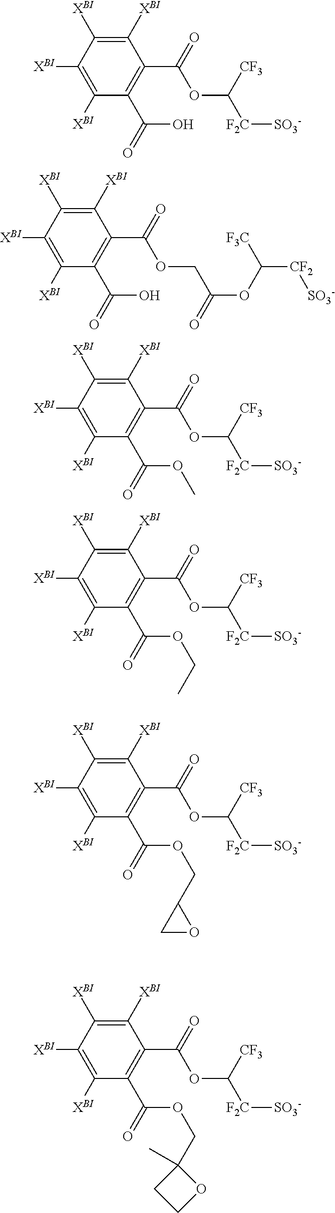

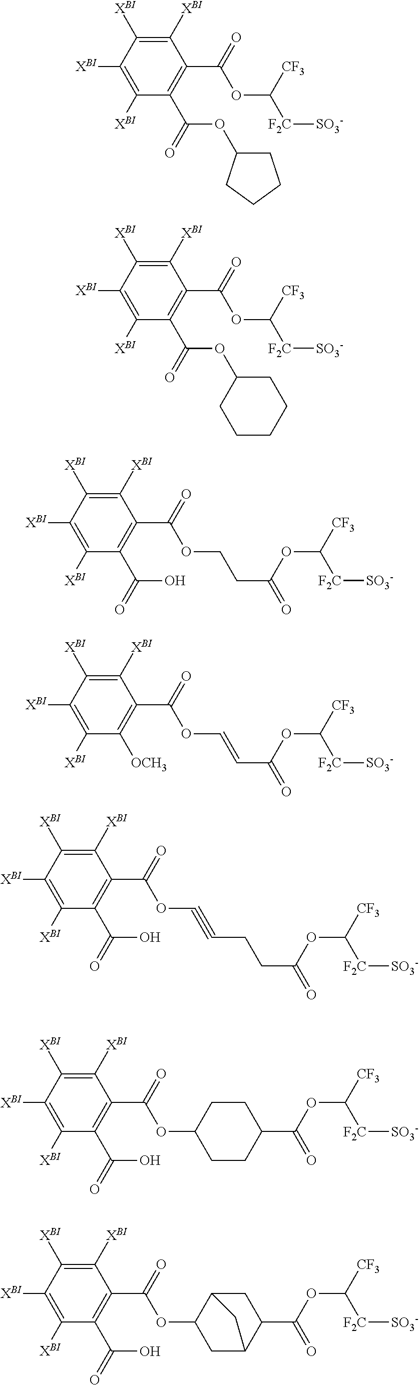

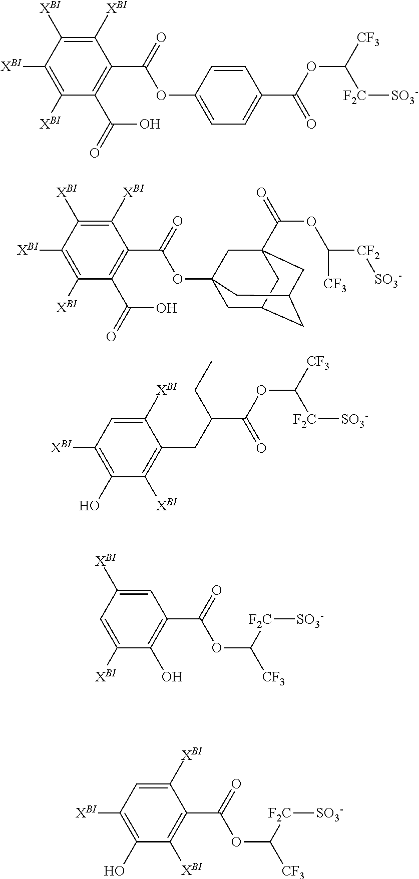

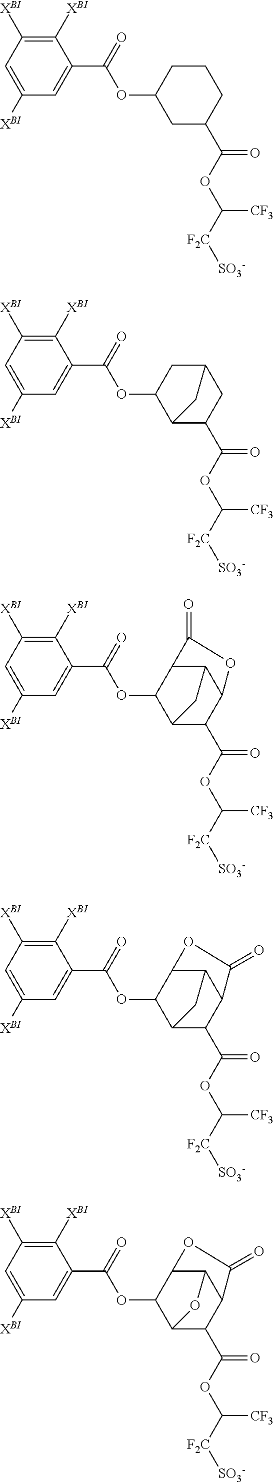

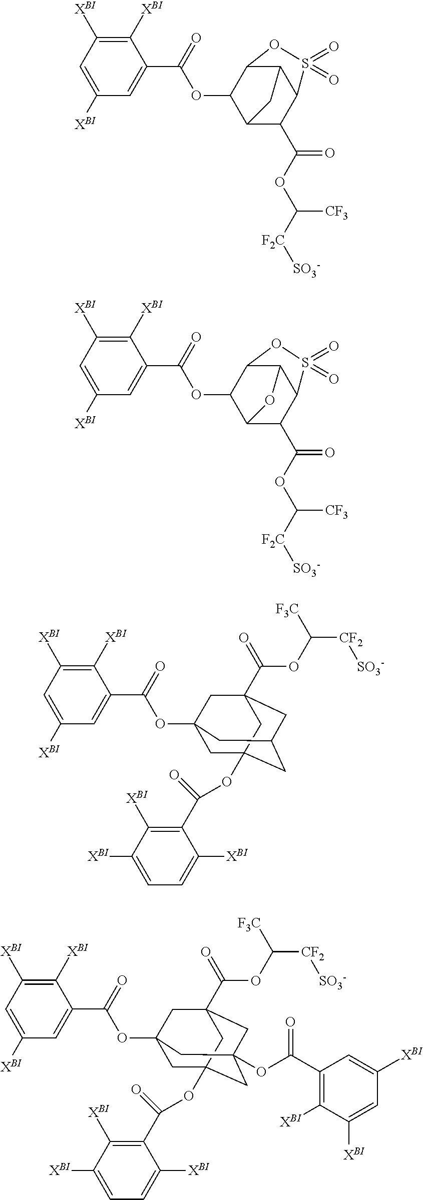

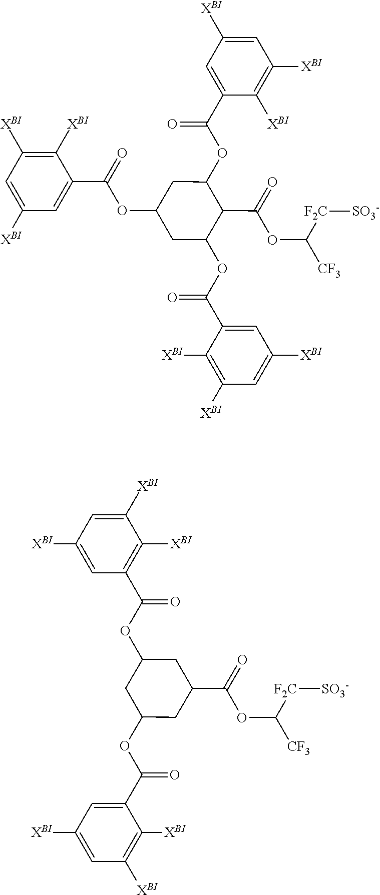

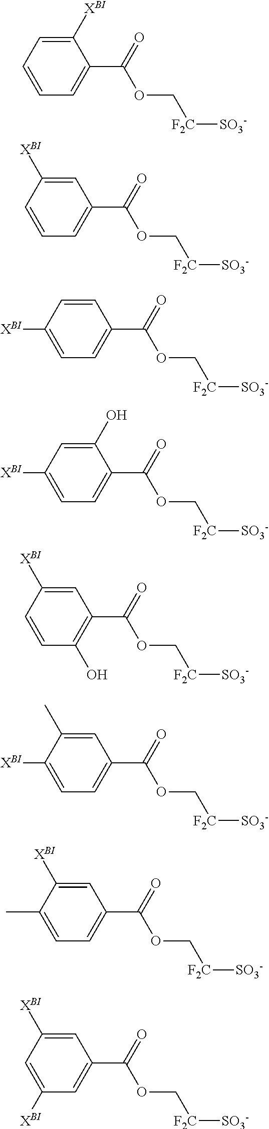

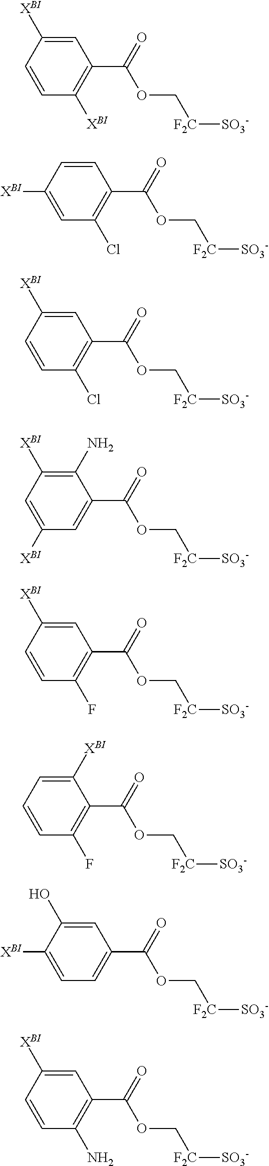

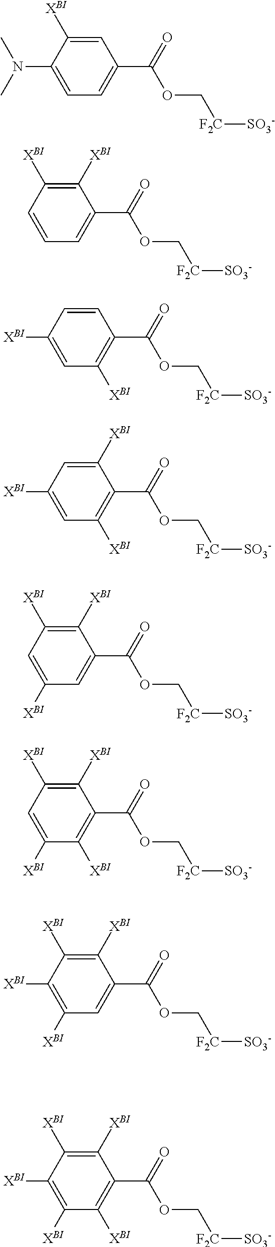

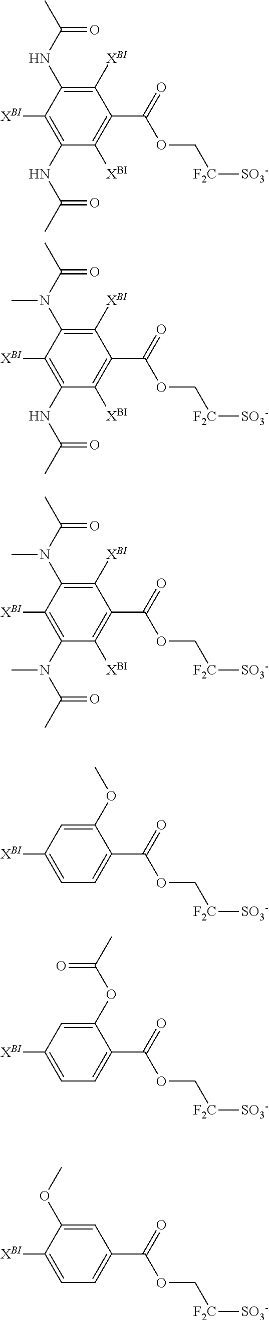

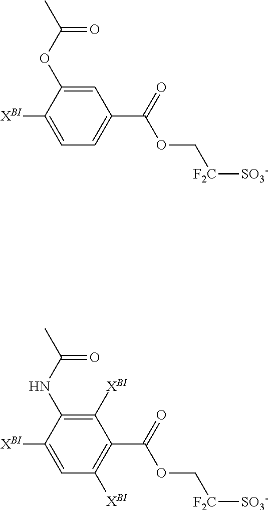

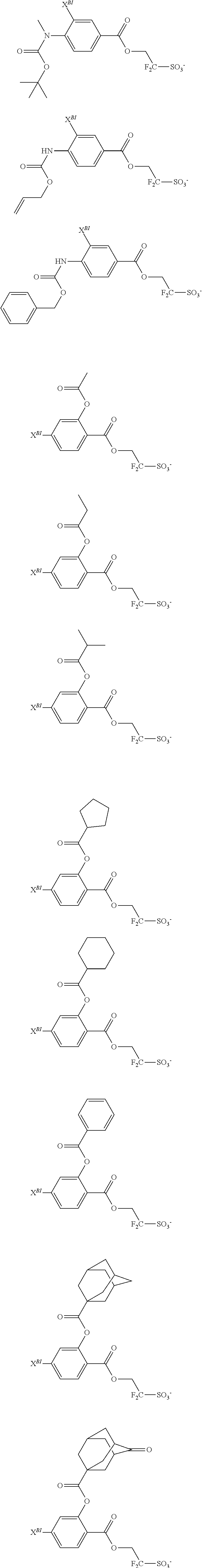

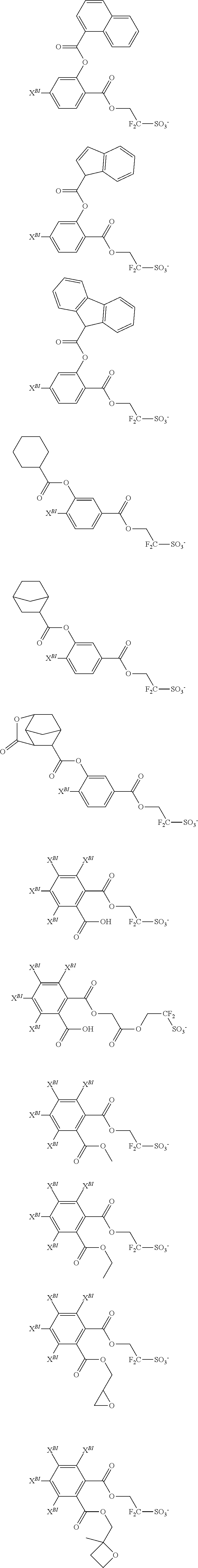

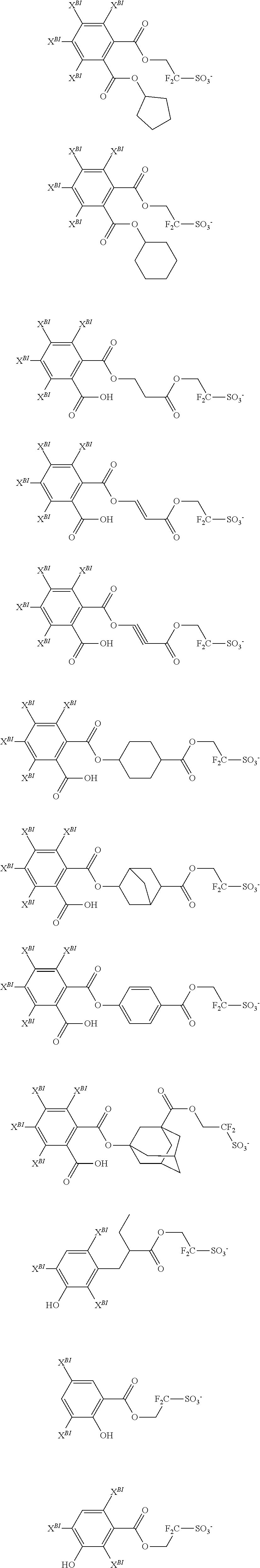

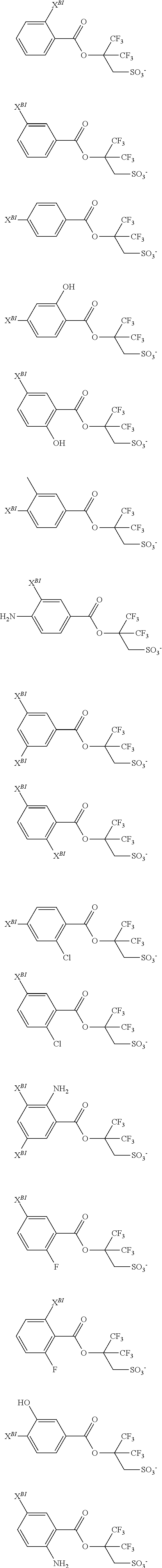

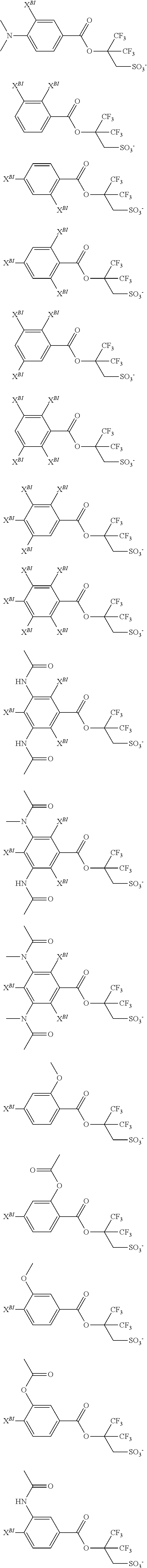

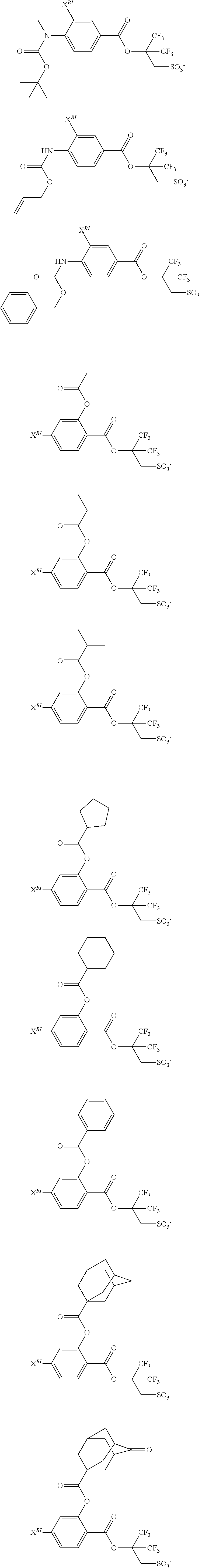

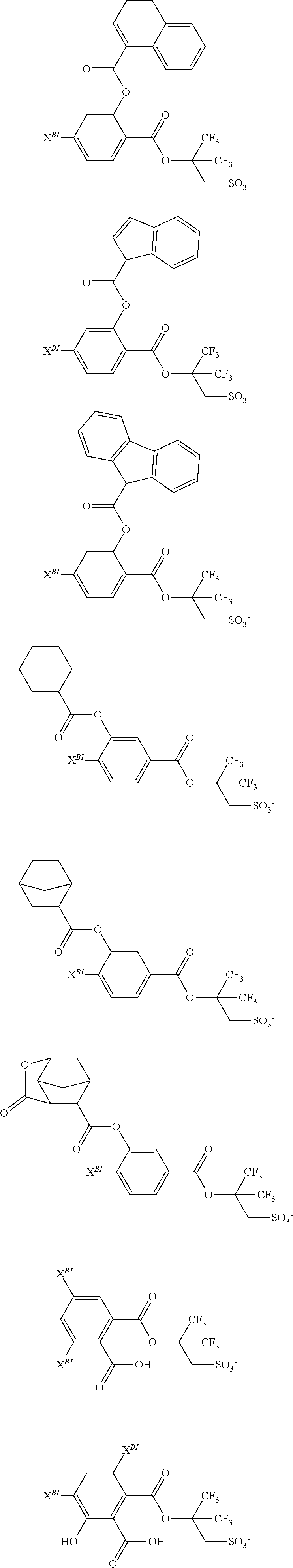

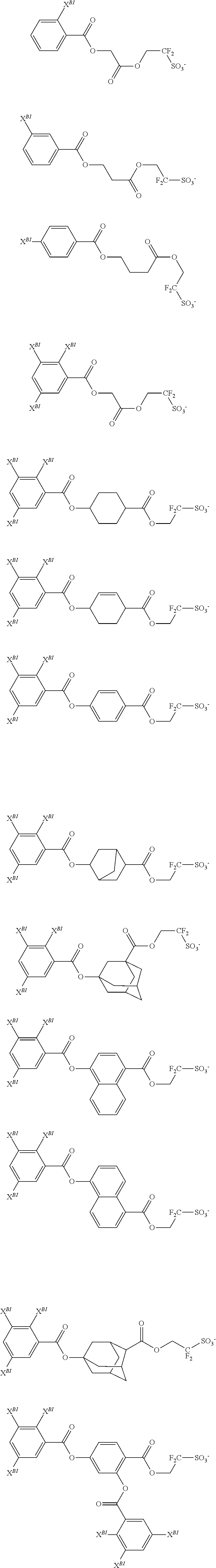

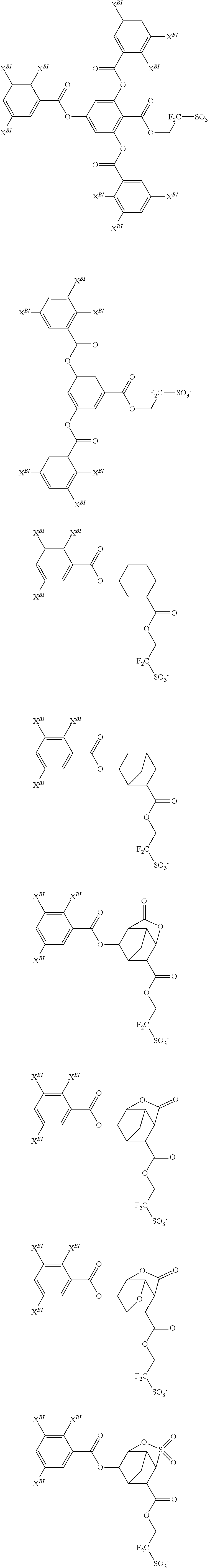

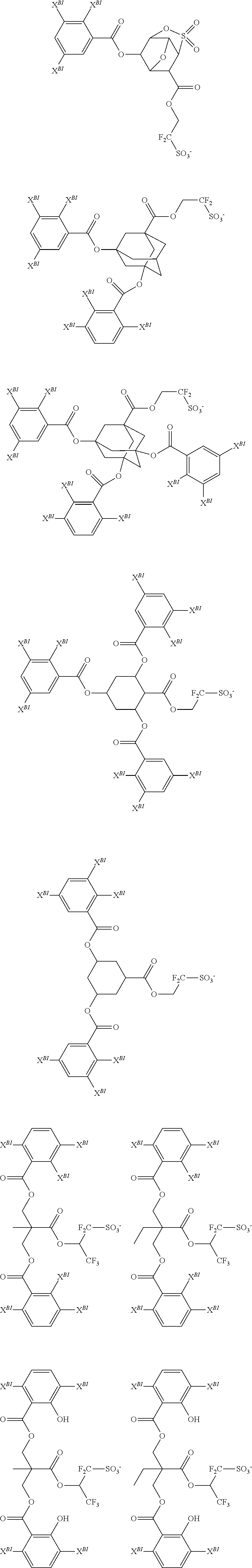

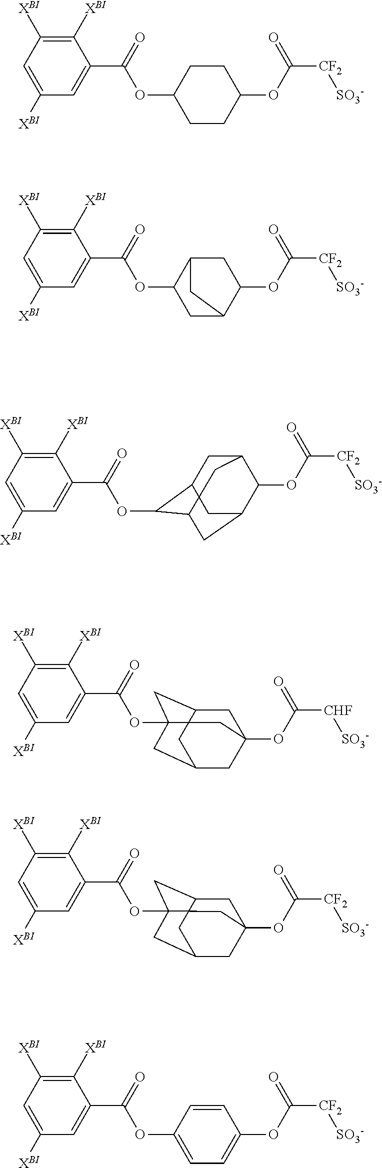

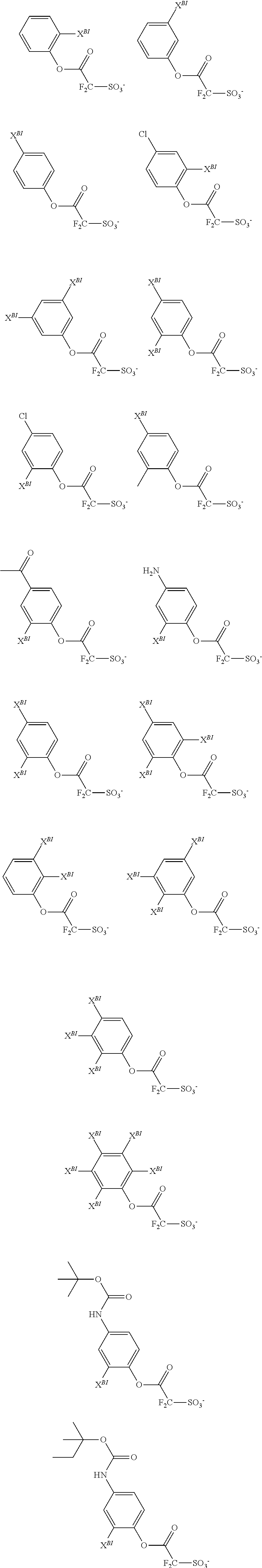

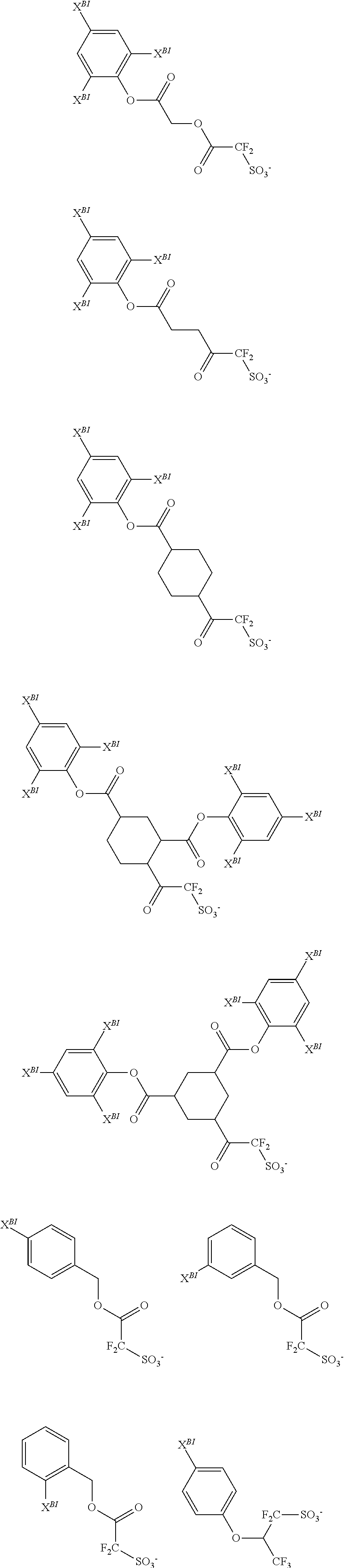

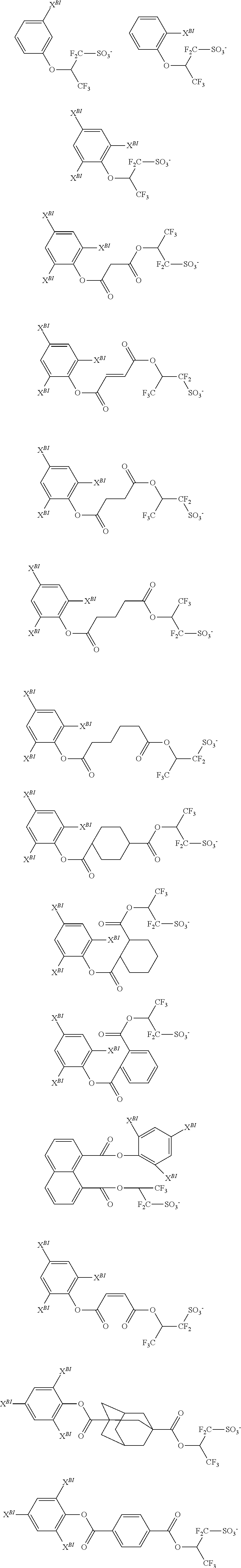

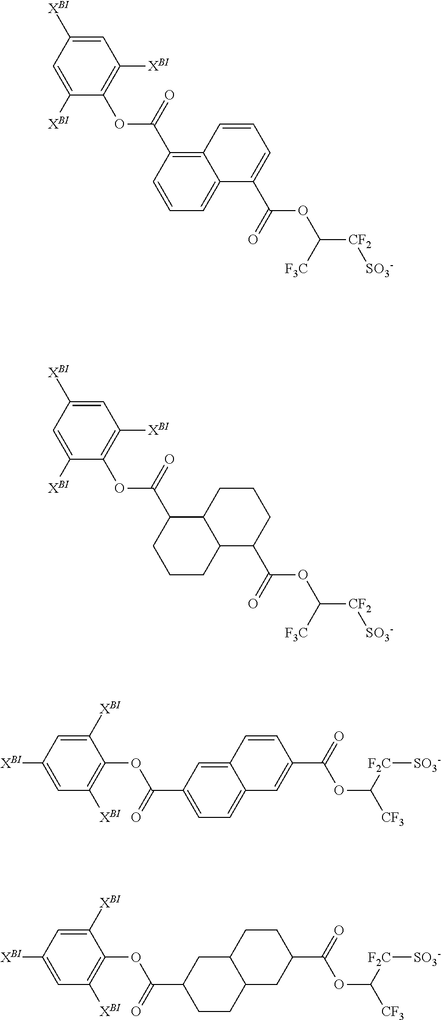

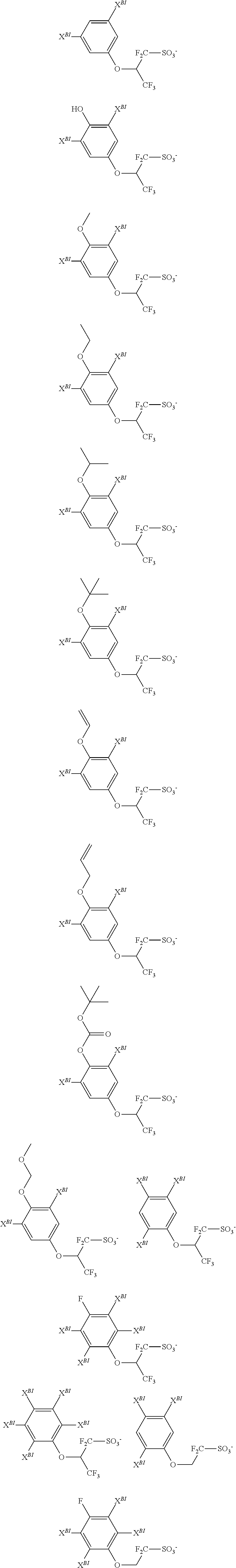

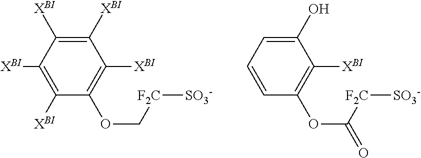

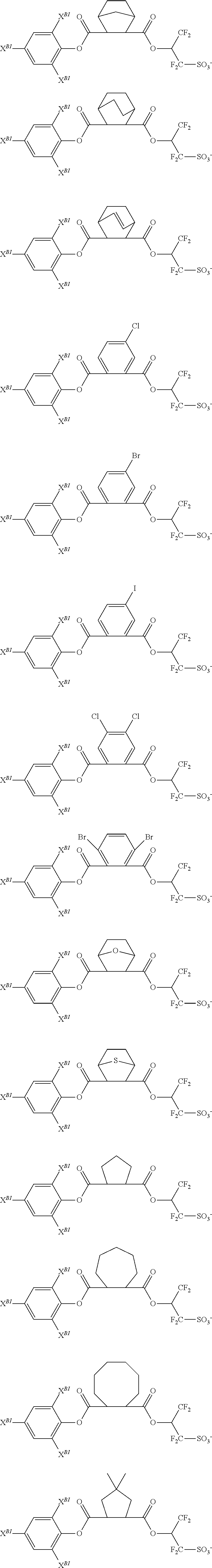

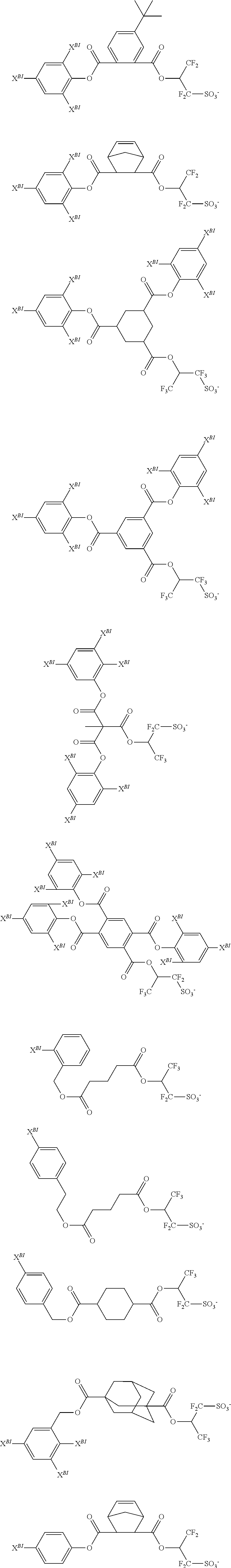

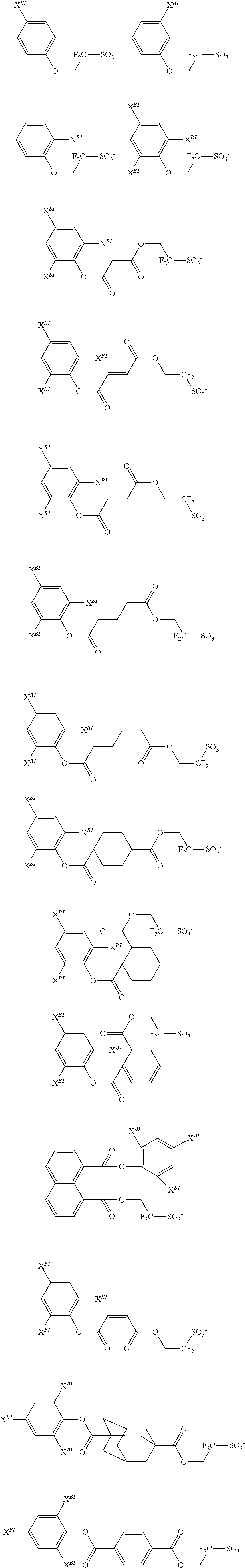

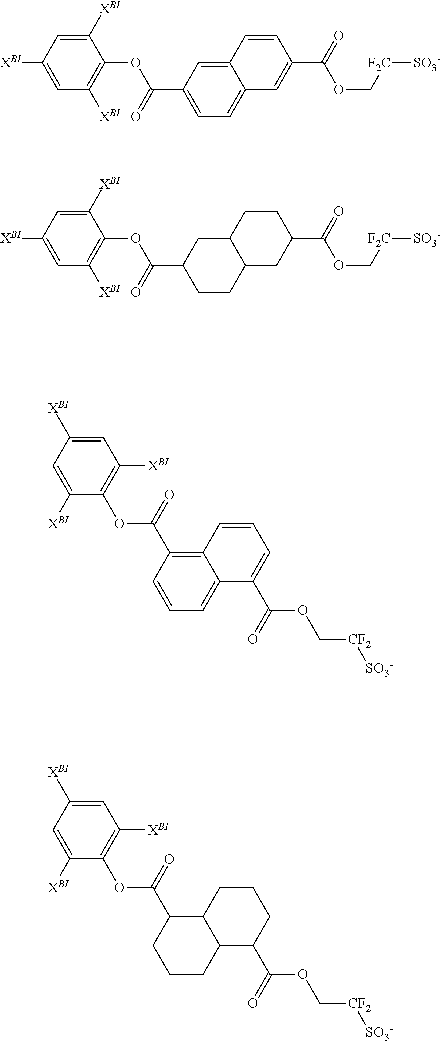

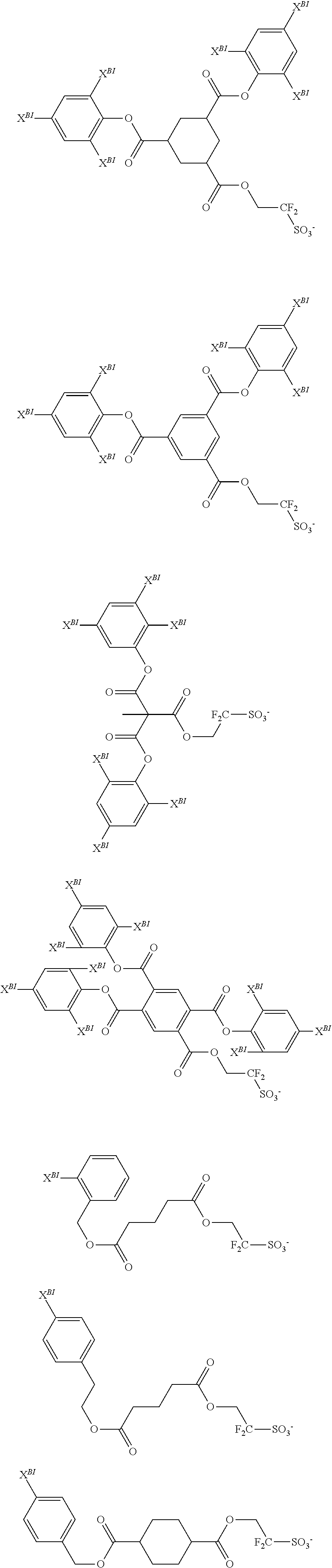

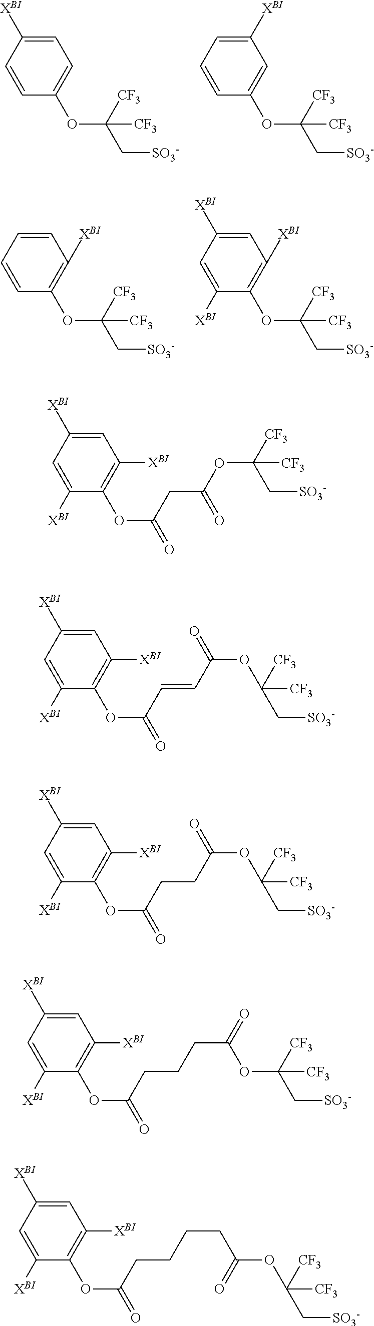

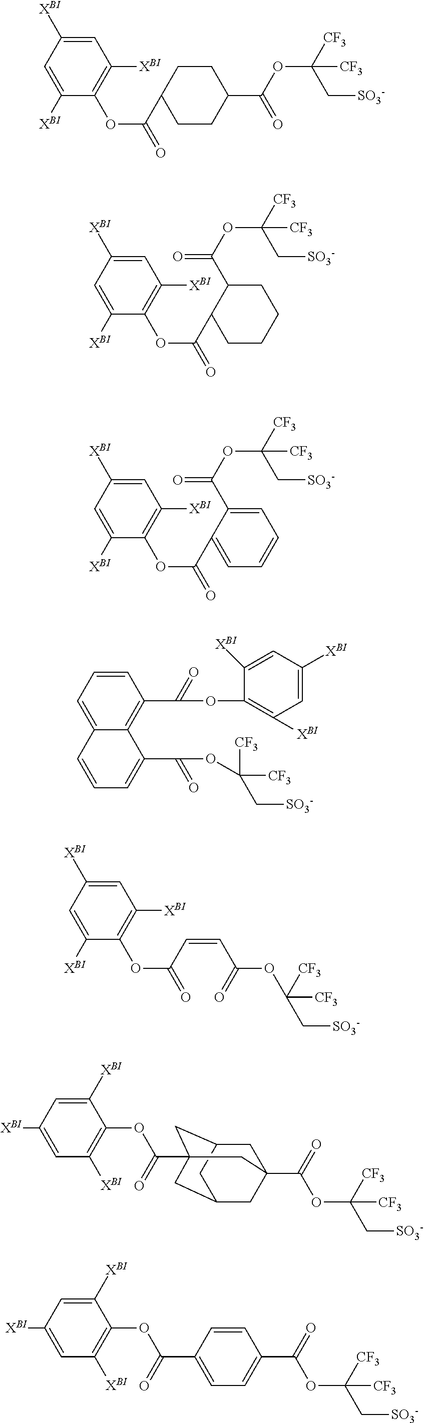

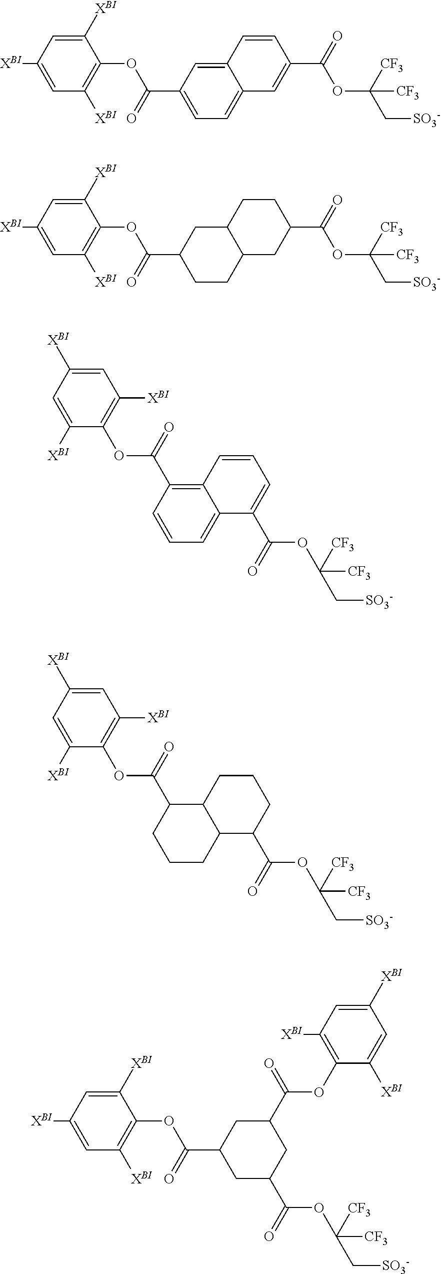

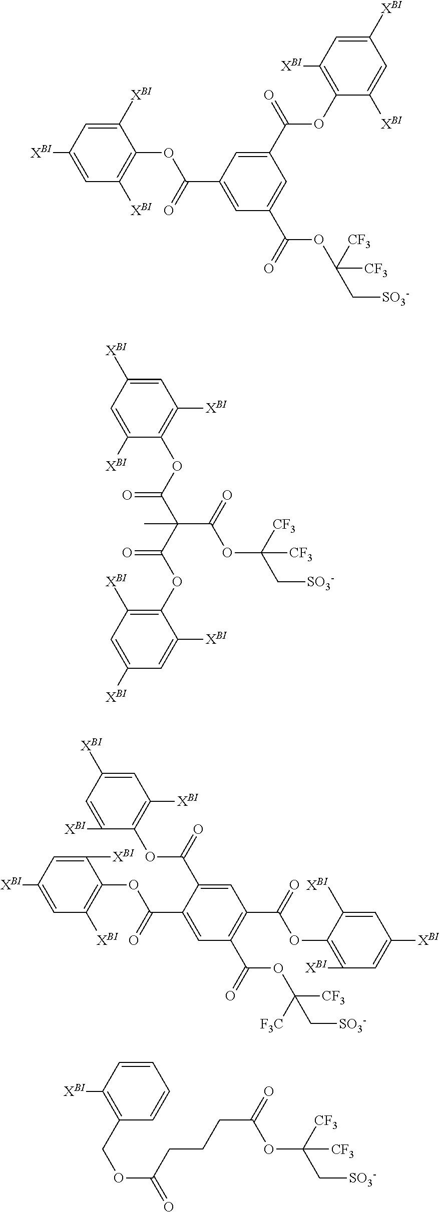

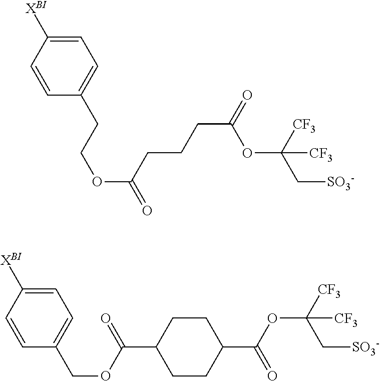

Other examples of the anion represented by Xa⁻ are as described in WO 2023/157455 paragraphs [0076] and [0106], WO 2024/024801, paragraph [0111], WO 2024/043121, paragraphs [0253]-[0256], WO 2024/057751, paragraphs [0044]-[0045], WO 2024/122423, paragraphs [0205]-[0220], JP-A 2023-123183, paragraphs [0170]-[0178], JP-A 2024-062406, paragraphs [0026]-[0028], JP-A 2024-062407, paragraphs [0022]-[0025], JP-A 2024-062408, paragraphs [0026]-[0028], JP-A 2024-068156, paragraphs [0028]-[0030], JP-A 2024-068157, paragraphs [0026]-[0028], JP-A 2024-068158, paragraphs [0028]-[0030], JP-A 2024-068159, paragraphs [0028]-[0030], JP-A 2024-072280, paragraphs [0031]-[0033], JP-A 2024-072281, paragraphs [0023]-[0025], JP-A 2024-077618, paragraphs [0026]-[0029], JP-A 2024-077619, paragraphs [0020]-[0021], JP-A 2024-080672, paragraphs [0140]-[0143], JP-A 2024-083303, paragraphs [0023]-[0025], JP-A 2024-083304, paragraphs [0028]-[0031], JP-A 2024-099500, paragraphs [0030]-[0033], JP-A 2024-099502, paragraphs [0028]-[0030], JP-A 2024-101557, paragraphs [0030]-[0032], JP-A 2024-102842, paragraphs [0025]-[0027], JP-A 2024-102843, paragraphs [0033]-[0035], JP-A 2024-127832, paragraphs [0021]-[0022], JP-A 2024-144354, paragraphs [0169]-[0172], JP-A 2024-144356, paragraphs [0178]-[0181], JP-A 2024-160436, paragraphs [0040]-[0143], JP 7247732, paragraphs [0157]-[0158], JP 7446352, paragraphs [0227]-[0238], JP 7466597, paragraphs [0253]-[0256], JP 7466782, paragraphs [0309]-[0312], and JP-A 2025-023601, paragraphs [0042]-[0058].

Other examples of the anion Xa⁻ include fluorine-free aromatic sulfonate anions and alkanesulfonate anions described in JP 7398551, paragraphs [0186]-[0188], JP 7067271, paragraphs [0028]-[0029], JP 7032549, paragraphs [0220]-[0225], JP 6645464, paragraphs [0040]-[0045], JP 6248882, paragraphs [0027]-[0029], JP 6090585, paragraphs [0034]-[0037], JP-A 2024-104830, paragraphs [0037]-[0060], JP-A 2024-140135, paragraphs [0033]-[0093], JP-A 2023-177038, paragraphs [0039]-[0066], and JP-A 2006-276759, paragraphs [0100]-[0102]. Examples thereof include fluorine-containing aromatic sulfonate anions described in JP 6648726, paragraphs [0029]-[0034], JP-A 2024-077330, paragraphs [0264]-[0267], and JP-A 2025-000201, paragraphs [0040]-[0071].

Other useful anions Xa⁻ include bis-sulfonate anions described in JP-A 2015-206932, sulfonamide or sulfonimide anions having sulfonate on one side and non-sulfonate on the other side as described in WO 2020/158366, and anions having sulfonate on one side and carboxylate on the other side as described in JP-A 2015-024989.

Photoacid generators (D) having the formula (5) are also preferred.

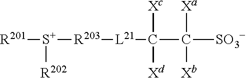

In formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with a sulfur atom to which they are attached.

The C₁-C₃₀ hydrocarbyl groups R²⁰¹ and R²⁰² may be saturated or unsaturated, and may be straight, branched or cyclic. Examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl, and n-decyl groups; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl groups; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl groups; and combinations thereof. In the hydrocarbyl group, some or all of the hydrogen atoms may be substituted by a moiety containing a heteroatom such as oxygen, sulfur, nitrogen, or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—), or haloalkyl moiety.

The C₁-C₃₀ hydrocarbylene group R²⁰³ may be saturated or unsaturated, and may be straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl groups; C₃-C₃₀ cyclic saturated hydrocarbylene groups such as cyclopentanediyl, cyclohexanediyl, norbornanediyl, and adamantanediyl groups; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene, and tert-butylnaphthylene groups. Some or all of hydrogen atoms of the hydrocarbylene group may be replaced by a group containing a heteroatom such as oxygen, sulfur, nitrogen, or halogen, some constituent —CH₂— of the hydrocarbylene group may be replaced by a group containing a heteroatom such as oxygen, sulfur or nitrogen, and as a result, the hydrocarbylene group may contain hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—), or haloalkyl moiety. The heteroatom is preferably oxygen.

In formula (5), L²¹ is a single bond, ether bond, or a C₁-C₂₀ hydrocarbylene group which may contain a heteroatom. The hydrocarbylene group may be saturated or unsaturated, and may be straight, branched or cyclic. Examples thereof are as exemplified above for the hydrocarbylene group R²⁰³.

In formula (5), X^{a}, X^{b}, X^{c} and X^{d} are each independently hydrogen, fluorine, or trifluoromethyl. At least one of X^{a}, X^{b}, X^{c} and X^{d} is fluorine or trifluoromethyl.

Of the photoacid generators having formula (5), those having the formula (5′) are preferred.

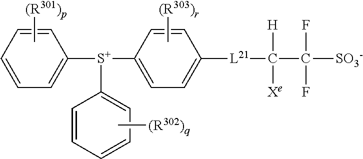

In formula (5′), L²¹ is as defined above. X^{e} is hydrogen or trifluoromethyl, preferably trifluoromethyl. R³⁰¹, R³⁰², and R³⁰³ are each independently hydrogen or a C₁-C₂₀ hydrocarbyl group which may contain a heteroatom. The hydrocarbyl group may be saturated or unsaturated, and may be straight, branched or cyclic. Examples thereof are as exemplified above for the hydrocarbyl group R^{fa1} in formula (Xa-1-1). p and q are each independently 0, 1, 2, 3, 4 or 5, and r is 0, 1, 2, 3 or 4.

Examples of the photoacid generator having formula (5) include those exemplified for the photoacid generator having formula (2) in JP-A 2017-026980.

Of the foregoing photoacid generators, those having an anion of formula (Xa-1-1) or (Xa-4) are especially preferred because of reduced acid diffusion and high solubility in solvents. Also those having formula (5′) are especially preferred because of extremely reduced acid diffusion.

When the chemically amplified resist composition comprises the photoacid generator (D), the amount is preferably 0.1 to 40 parts by weight, and more preferably 0.5 to 20 parts by weight per 80 parts by weight of the base polymer (A). As long as the amount of the photoacid generator (D) is in the range, good resolution is achievable and the risk of foreign matter being formed after development or during stripping of resist film is avoided. The photoacid generator (D) may be used alone or in admixture.

### (E) Surfactant

The chemically amplified resist composition may further comprise (E) a surfactant. Preferred are (E) a surfactant which is insoluble or substantially insoluble in water and soluble in alkaline developer, and a surfactant which is insoluble or substantially insoluble in water and alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are surfactants FC-4430 (3M), Surflon® 5-381 (AGC Seimi Chemical Co., Ltd.), Olfine® E1004 (Nissin Chemical Co., Ltd.), and KH-20 and KH-30 (AGC Seimi Chemical Co., Ltd.). Partially fluorinated oxetane ring-opened polymers having the formula (surf-1) are also useful.

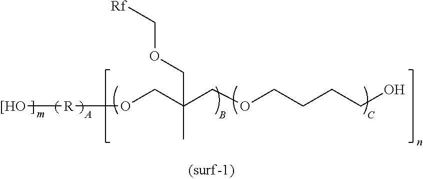

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

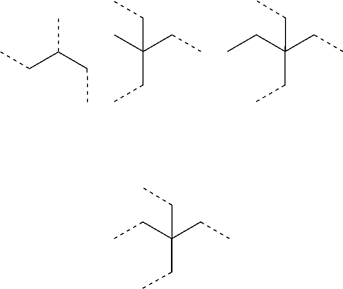

Herein, the broken line denotes a point of attachment. These formulae are partial structures derived from glycerol, trimethylol ethane, trimethylol propane, and pentaerythritol, respectively.

Of these, 1,4-butylene and 2,2-dimethyl-1,3-propylene are preferably used.

Rf is trifluoromethyl or pentafluoroethyl, and preferably trifluoromethyl. m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably 0 or 1. The formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure or PEB, and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Examples of the polymeric surfactant include those containing at least one type selected from repeat units having the formula (6A) (hereinafter, also referred to as repeat units 6A), repeat units having the formula (6B) (hereinafter, also referred to as repeat units 6B), repeat units having the formula (6C) (hereinafter, also referred to as repeat units 6C), repeat units having the formula (6D) (hereinafter, also referred to as repeat units 6D), and repeat units having the formula (6E) (hereinafter, also referred to as repeat units 6E).

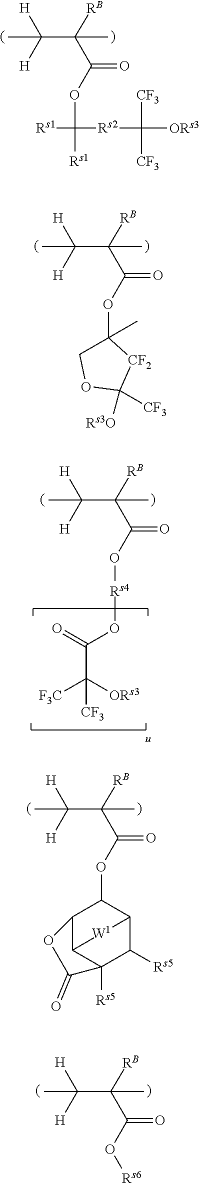

In formulae (6A) to (6E), R^{B} is hydrogen, fluorine, methyl or trifluoromethyl. W¹ is —CH₂—, —CH₂CH₂—, —O—, or two separate —H. R¹ is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group. u is 1, 2 or 3. R^{s5} is each independently hydrogen or a group: —C(═O)—O—R^{sa}. R^{sa} is a C₁-C₂₀ fluorinated hydrocarbyl group. R^{S6} is a C₁-C₁₅ hydrocarbyl or fluorinated hydrocarbyl group in which an ether bond or carbonyl moiety may intervene in a carbon-carbon bond.

The C₁-C₁₀ hydrocarbyl group of R^{s1} is preferably a saturated hydrocarbyl group, and may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl, n-decyl, and C₃-C₁₀ cyclic saturated hydrocarbyl groups such as cyclopropyl, cyclobutyl, cyclopentyl, cyclohexyl, adamantyl, and norbornyl. Inter alia, C₁-C₆ hydrocarbyl groups are preferred.

The hydrocarbylene group of R^{s2} is preferably a saturated hydrocarbylene group, and may be straight, branched or cyclic. Examples thereof include methylene, ethylene, propylene, butylene, and pentylene.

The hydrocarbyl group represented by R^{s3} or R^{S6} may be saturated or unsaturated, and may be straight, branched or cyclic. Examples thereof include saturated hydrocarbyl groups, and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic and examples thereof include the foregoing hydrocarbyl or fluorinated hydrocarbyl groups from which “u” number of hydrogen atoms are eliminated.

The fluorinated hydrocarbyl group represented by R^{sa} may be straight, branched or cyclic and is preferably saturated. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units 6A to 6E are shown below, but are not limited thereto. Herein, R^{B} is as defined above.

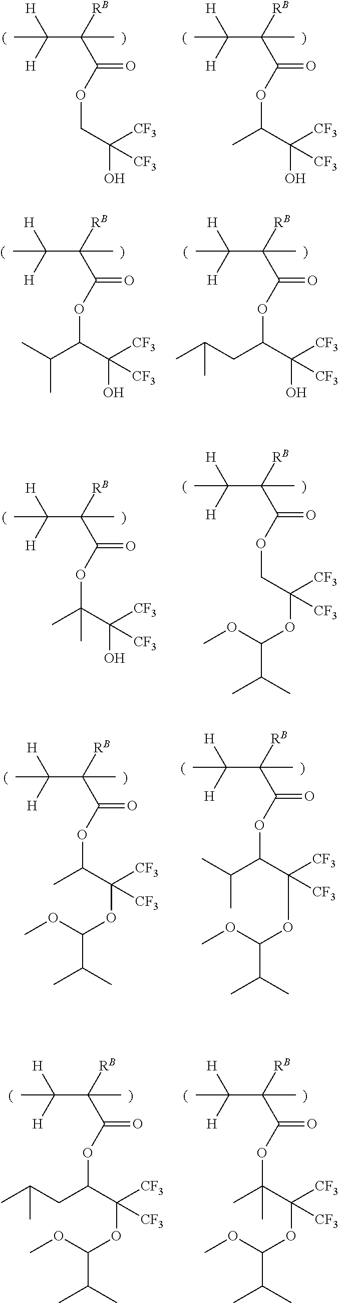

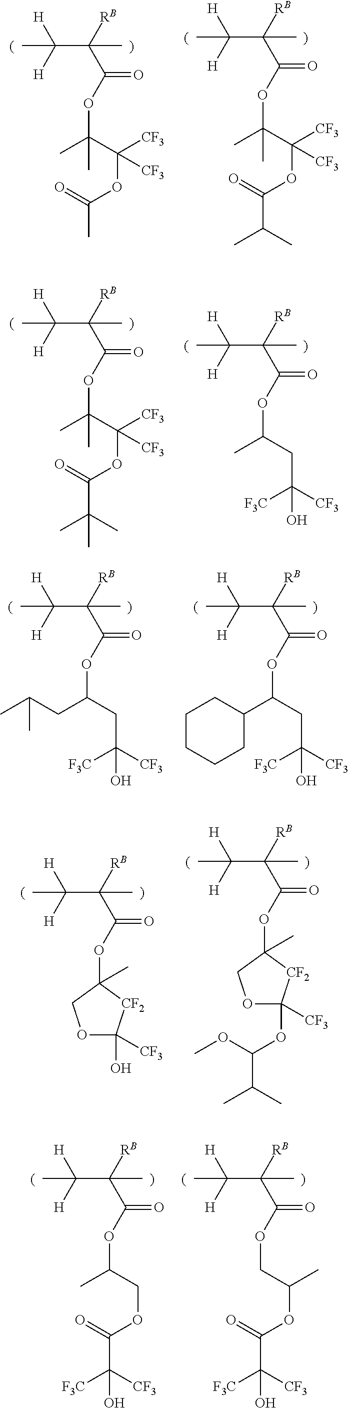

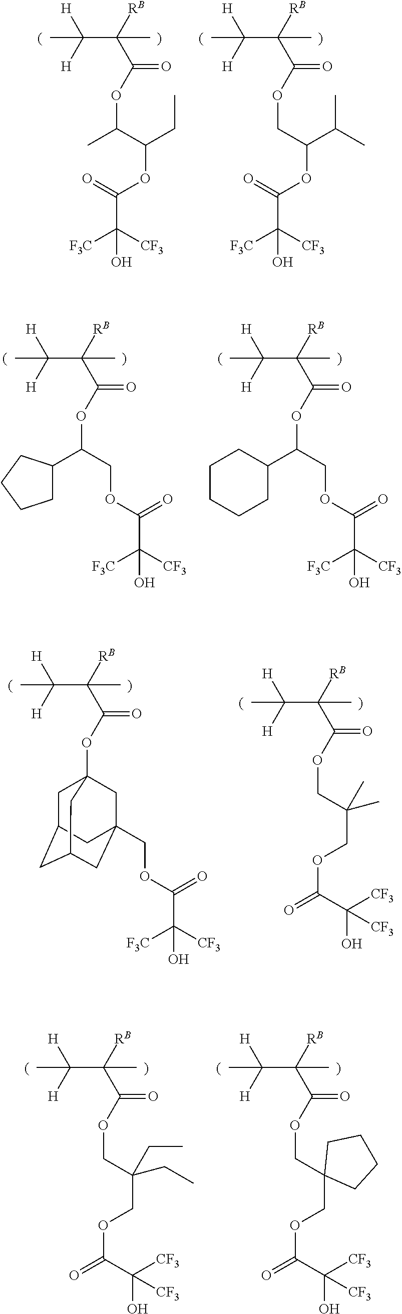

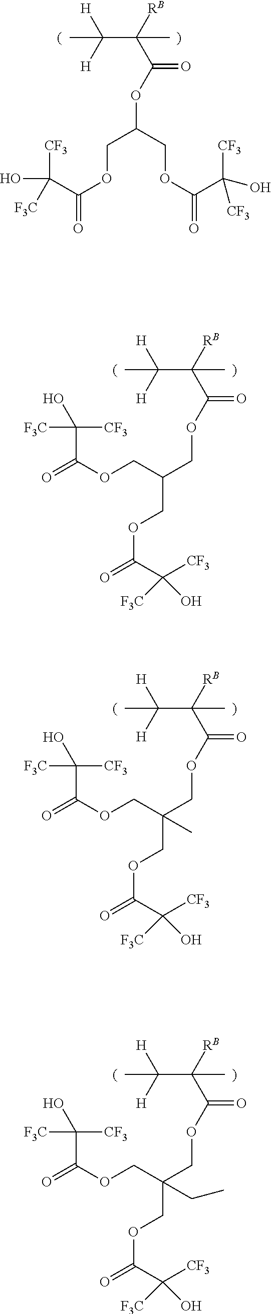

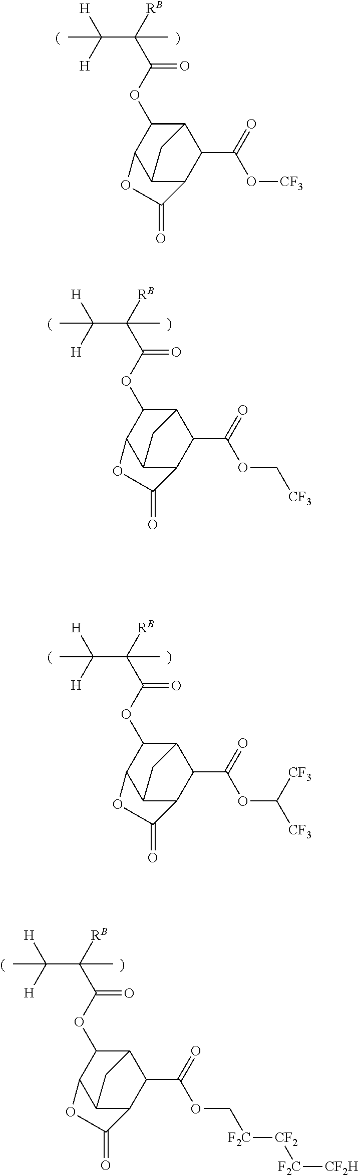

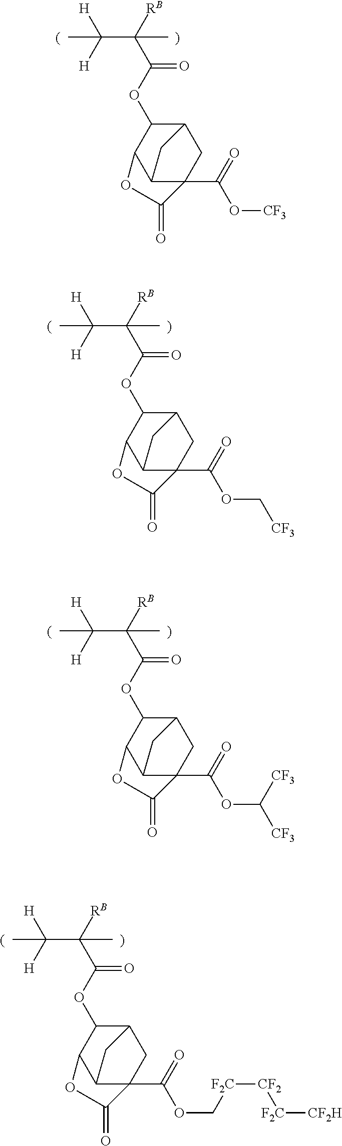

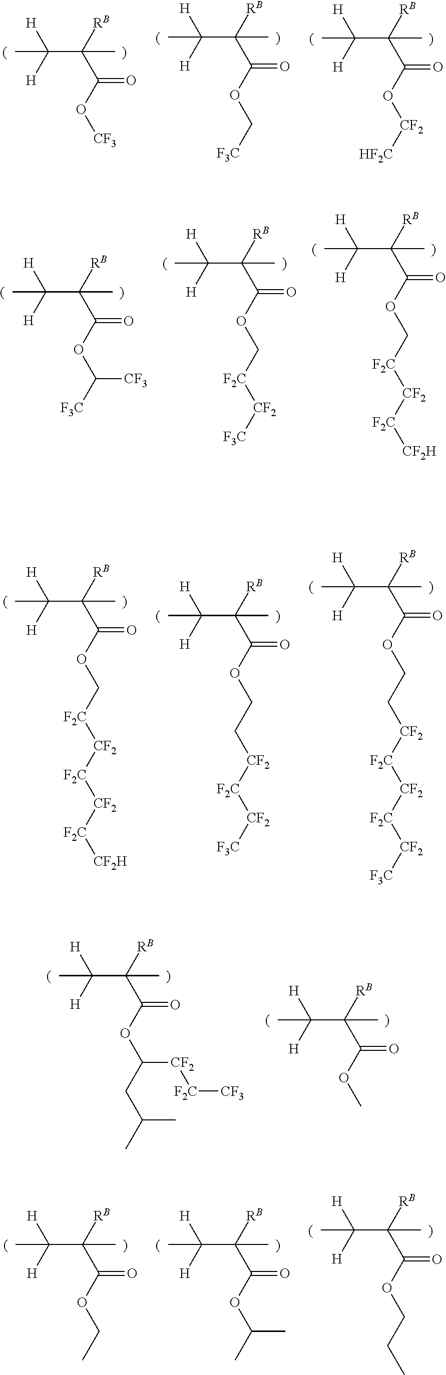

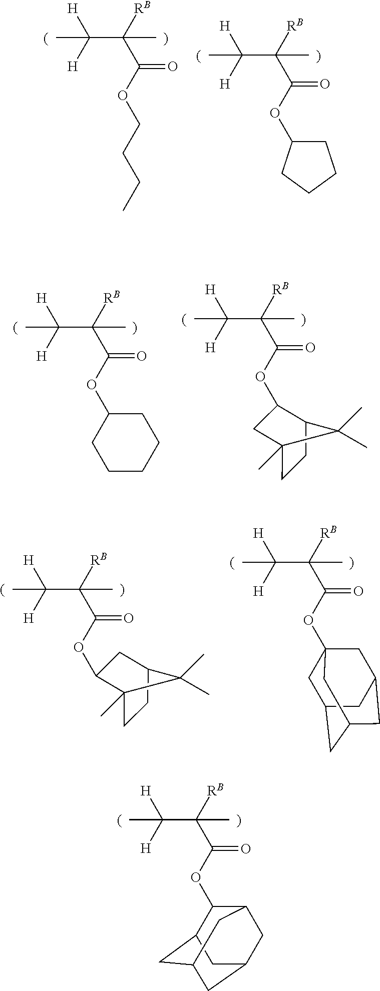

The polymeric surfactant may further contain repeat units other than the repeat units 6A to 6E. Typical other repeat units are those derived from methacrylic acid and α-trifluoromethylacrylic acid derivatives. In the polymeric surfactant, the content of the repeat units 6A to 6E is preferably at least 20 mol %, more preferably at least 60 mol %, most preferably 100 mol % of the overall repeat units.

The polymeric surfactant preferably has a Mw of 1,000 to 500,000, more preferably 3,000 to 100,000, and a Mw/Mn of 1.0 to 2.0, more preferably 1.0 to 1.6.

The polymeric surfactant may be synthesized by any desired method, for example, by dissolving an unsaturated bond-containing monomer or monomers providing at least one repeat unit selected from repeat units 6A to 6E and optionally other repeat units in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

During the synthesis of polymeric surfactant, any known chain transfer agent such as dodecyl mercaptan or 2-mercaptoethanol may be added for molecular weight control purpose. The amount of chain transfer agent added is preferably 0.01 to 10 mol % based on the total moles of monomers to be polymerized.

When the chemically amplified resist composition contains a surfactant (E), the amount thereof is preferably 0.1 to 50 parts by weight, and more preferably 0.5 to 10 parts by weight per 80 parts by weight of the base polymer (A). At least 0.1 parts by weight of the surfactant (E) is effective in improving the receding contact angle with water of the resist film at its surface. Up to 50 parts by weight of the surfactant is effective in forming a resist film having a low rate of dissolution in a developer and capable of maintaining the height of a small-size pattern formed therein. The surfactant (E) may be used alone or in admixture.

### (F) Other Components

The chemically amplified resist composition may further comprise (F) other components, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). Specifically, the acid amplifier compound is described in JP-A 2009-269953 and JP-A 2010-215608 and preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the remaining additives, reference should be made to JP-A 2009-269953 and JP-A 2010-215608.

### [Pattern Forming Process]

A pattern forming process of the invention includes: applying the chemically amplified resist composition onto a substrate to form a resist film thereon; exposing the resist film to high-energy radiation; and developing the exposed resist film in a developer.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The chemically amplified resist composition is applied onto a substrate by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of 0.05 to 2 μm.

Then the resist film is exposed to a pattern of high-energy radiation, typically KrF or ArF excimer laser, EUV of wavelength 3 to 15 nm or EB. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. The liquid is typically water, and in this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

After the exposure, the resist film may be baked (PEB), for example, on a hotplate preferably at 60 to 150° C. for 1 to 5 minutes, more preferably at 80 to 140° C. for 1 to 3 minutes.

The resist film is then developed with a developer in the form of an aqueous base solution, for example, 0.1 to 5 wt %, preferably 2 to 3 wt % aqueous solution of tetramethylammonium hydroxide (TMAH) for 0.1 to 3 minutes, preferably 0.5 to 2 minutes by conventional techniques such as dip, puddle and spray techniques. In this way, the exposed region of the resist film is dissolved away, and a desired resist pattern is formed on the substrate.

Any desired step may be added to the pattern forming process. For example, after the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents may be used alone or in admixture.

### EXAMPLES

Synthesis Examples, Examples, and Comparative Examples are given below by way of illustration and not by way of limitation. The apparatuses used are as follows.

MALDI TOF-MS: S3000 manufactured by JEOL Ltd.

### [1] Synthesis of Sulfonium Salt Monomer

### Example 1-1

### Synthesis of Monomer PAG-1

### (1) Synthesis of Intermediate In-1

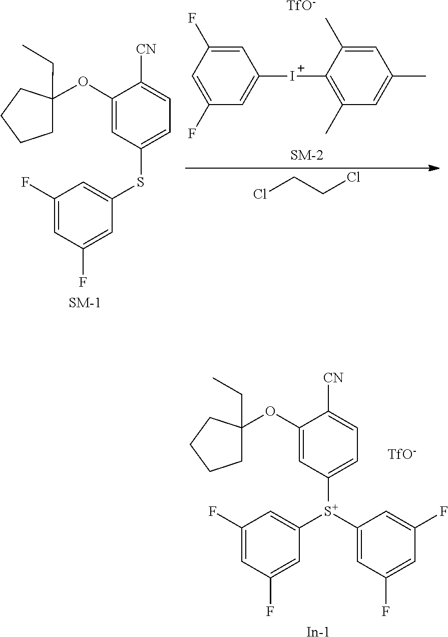

In nitrogen atmosphere, 71.9 g of Compound SM-1 and 122.0 g of Compound SM-2 were dissolved in 400 g of 1,2-dichloroethane. Thereafter, the solution was heated to reflux for 15 hours. The reaction solution was cooled to 25° C., and 100 g of water was added to quench the reaction. Thereafter, the organic layer was taken out, washed with water, and concentrated under reduced pressure to distill off the solvent. Purification of the residue by silica gel chromatography gave 75.8 g (yield 61%) of Intermediate In-1 as colorless oily matter.

### (2) Synthesis of Monomer PAG-1

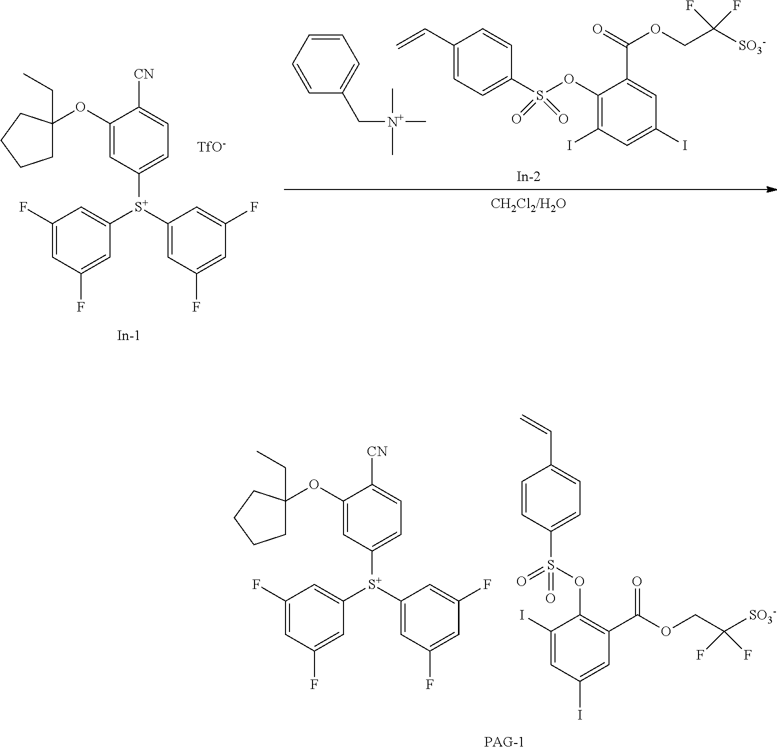

In nitrogen atmosphere, a reactor was charged with 55.9 g of Intermediate In-1, 76.4 g of Intermediate In-2, 300 g of methylene chloride, and 150 g of water. The mixture was stirred for 30 minutes at room temperature. The organic layer was taken out, washed with water, and concentrated under reduced pressure. Purification of the residue by silica gel chromatography gave 97.0 g (yield 92%) of Monomer PAG-1 as oily matter.

PAG-1 was analyzed by TOF-MS, with the data shown below.

### MALDI TOF-MS:

POSITIVE M⁺472 (corresponding to C₂₆H₂₂F₄NOS⁺)
NEGATIVE M⁻699 (corresponding to C₁₇H₁₁F₂I₂O₈S₂⁻)

### Examples 1-2 to 1-9

### Synthesis of Monomers PAG-2 to PAG-9

Sulfonium salt monomers PAG-2 to PAG-9 shown below were synthesized using corresponding reactants and well-known organic synthesis reactions.

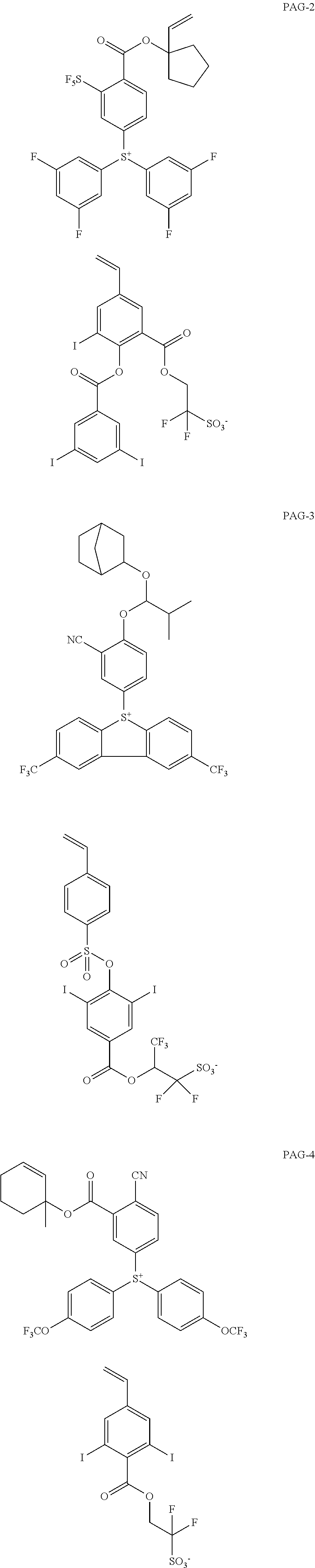

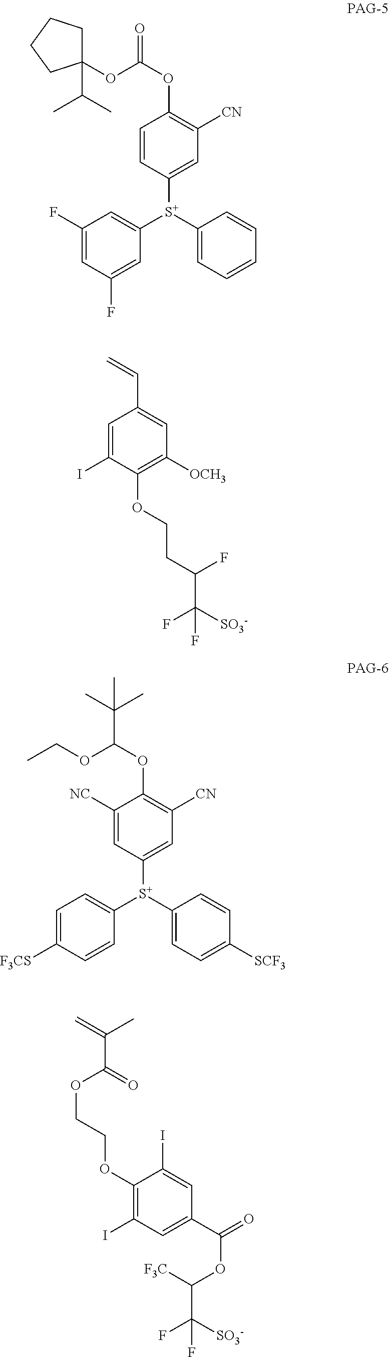

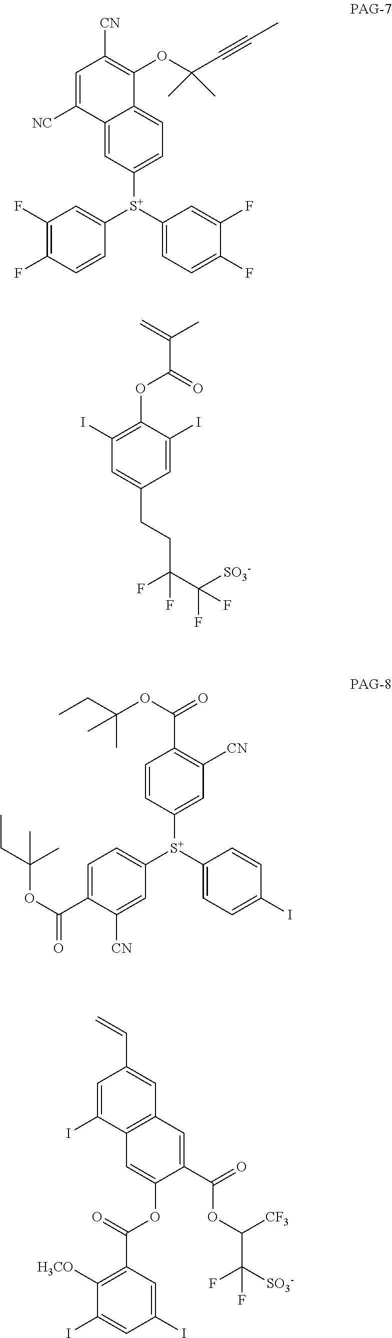

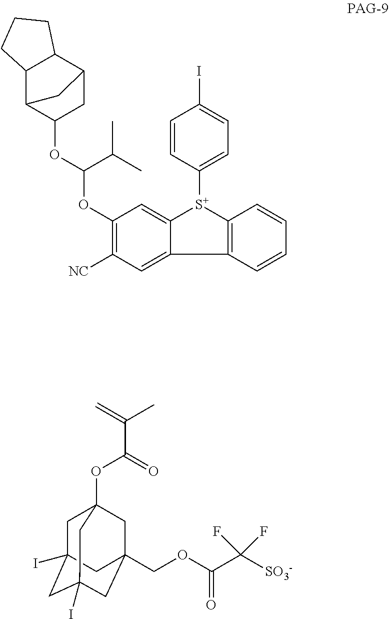

### Comparative Examples 1-1 to 1-6

### Synthesis of Comparative Monomers PAG-A to PAG-F

Comparative sulfonium salt monomers PAG-A to PAG-F shown below were synthesized using corresponding reactants and well-known organic synthesis reactions.

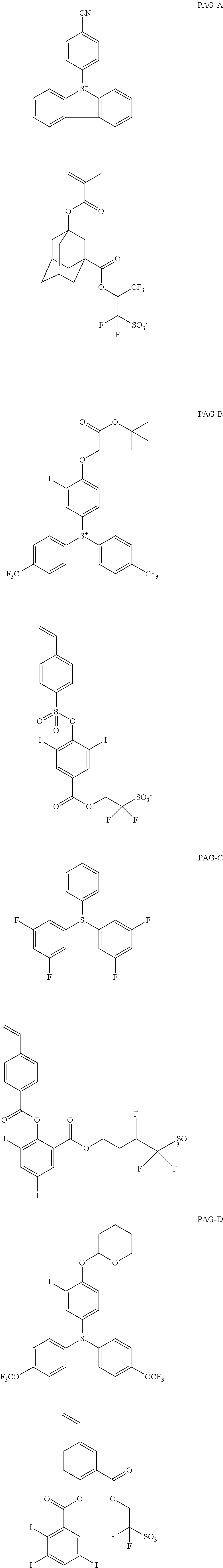

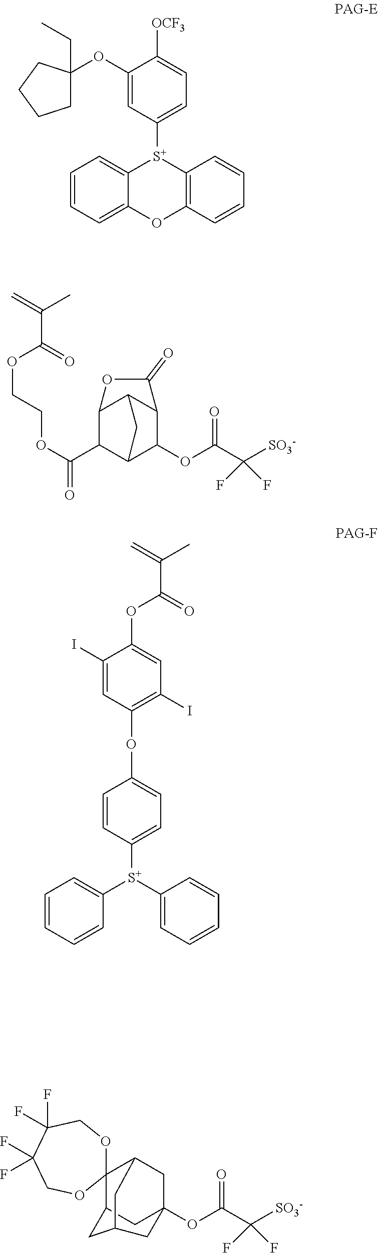

### [2] Synthesis of Base Polymer

In addition to monomers PAG-1 to PAG-9 and PAG-A to PAG-F, the following monomers were used for the synthesis of base polymers.

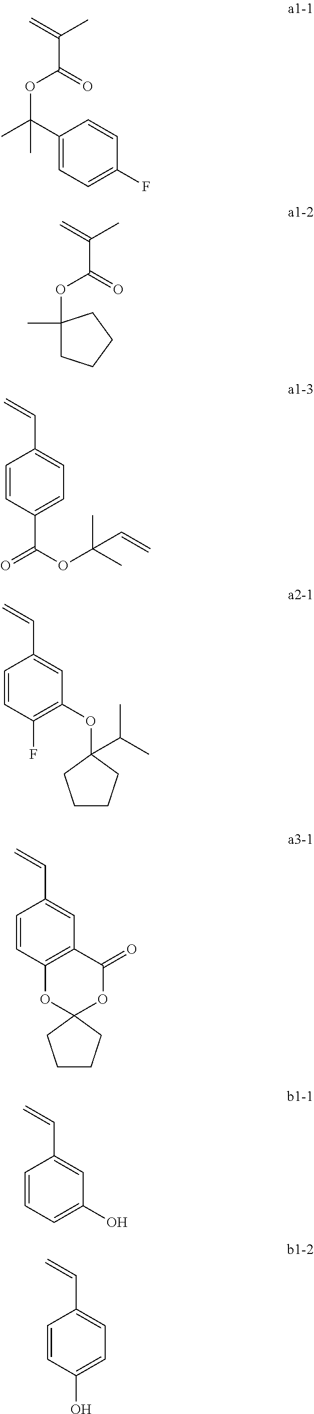

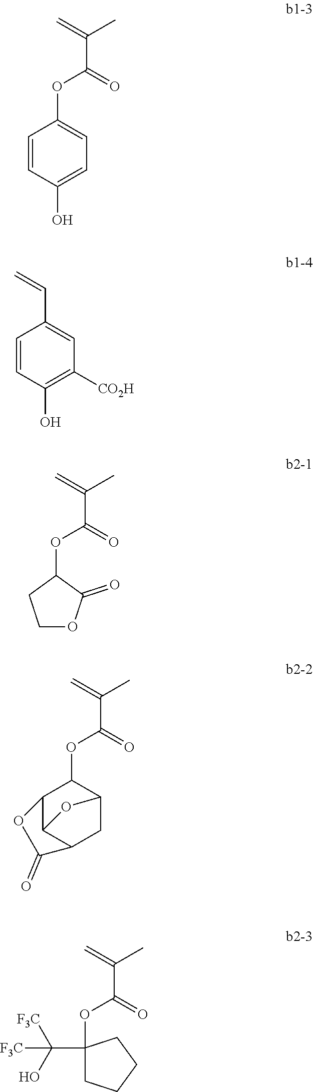

### Example 2-1

### Synthesis of Polymer P-1

A flask under nitrogen atmosphere was charged with 36.8 g of Monomer a1-1, 10.8 g of Monomer b1-1, 52.6 g of Monomer PAG-1, 3.45 g of V-601 (manufactured by Wako Pure Chemical Industries, Ltd.), and 140 g of MEK to prepare a monomer/initiator solution. Another flask under nitrogen atmosphere was charged with 46 g of MEK, which was heated to 80° C. with stirring. The monomer/initiator solution was added dropwise to the MEK over 4 hours. At the end of dropwise addition, the polymerization solution was continuously stirred for 2 hours while maintaining the temperature of 80° C. The polymerization solution was cooled to room temperature. The obtained polymerization solution was added dropwise to 3,000 g of hexane with vigorous stirring. The precipitate was collected by filtration. The precipitate was washed twice with 600 g of hexane and vacuum dried at 50° C. for 20 hours to obtain Polymer P-1 as white powder (amount 97.3 g, yield 97%). The polymer P-1 had a value of Mw of 9,300, and a value of Mw/Mn of 1.51. It is noted that Mw is measured by GPC versus polystyrene standards using DMF solvent.

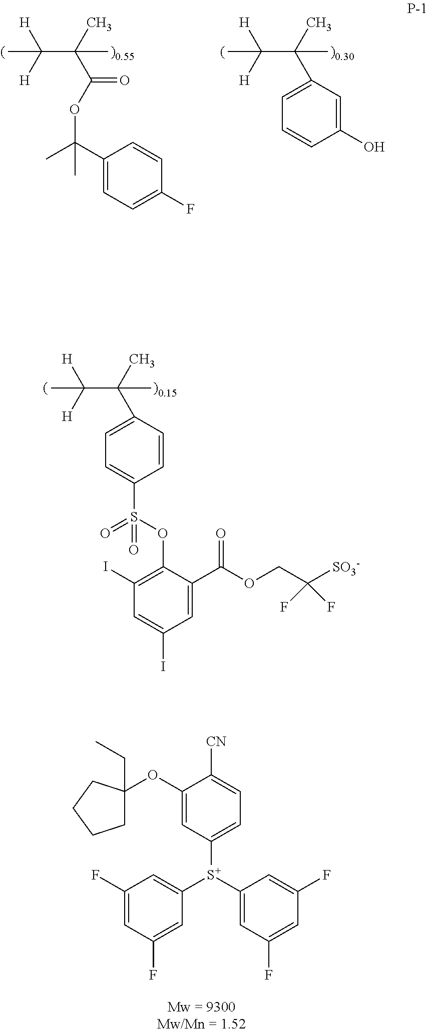

### Examples 2-2 to 2-26 and Comparative Examples 2-1 to 2-18

### Synthesis of Polymers P-2 to P-26 and CP-1 to CP-18

Polymers shown in Tables 1 and 2 were synthesized by the same procedure as in Example 2-1 except that the type and amount (blending ratio) of monomers were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | PAG-1 | 15 | a1-1 | 55 | b1-1 | 30 | — | — | — | — | 9,200 | 1.52 |
| P-2 | PAG-2 | 15 | a1-1 | 55 | b1-1 | 30 | — | — | — | — | 9,100 | 1.51 |
| P-3 | PAG-3 | 15 | a1-1 | 55 | b1-1 | 30 | — | — | — | — | 9,300 | 1.54 |
| P-4 | PAG-4 | 15 | a1-1 | 55 | b1-1 | 30 | — | — | — | — | 9,200 | 1.51 |
| P-5 | PAG-5 | 15 | a1-1 | 55 | b1-1 | 30 | — | — | — | — | 9,100 | 1.51 |
| P-6 | PAG-6 | 15 | a1-1 | 55 | b1-1 | 30 | — | — | — | — | 9,500 | 1.52 |
| P-7 | PAG-7 | 15 | a1-1 | 55 | b1-1 | 30 | — | — | — | — | 9,300 | 1.51 |
| P-8 | PAG-8 | 15 | a1-1 | 55 | b1-1 | 30 | — | — | — | — | 9,200 | 1.52 |
| P-9 | PAG-9 | 15 | a1-1 | 55 | b1-1 | 30 | — | — | — | — | 9,400 | 1.51 |
| P-10 | PAG-1 | 15 | a1-2 | 55 | b1-1 | 30 | — | — | — | — | 9,600 | 1.52 |
| P-11 | PAG-1 | 15 | a1-3 | 55 | b1-1 | 30 | — | — | — | — | 9,400 | 1.51 |
| P-12 | PAG-1 | 15 | a2-1 | 55 | b1-1 | 30 | — | — | — | — | 9,600 | 1.53 |
| P-13 | PAG-1 | 15 | a3-1 | 45 | b1-1 | 40 | — | — | — | — | 9,200 | 1.51 |
| P-14 | PAG-2 | 15 | a1-1 | 55 | b1-2 | 30 | — | — | — | — | 9,100 | 1.50 |
| P-15 | PAG-2 | 15 | a1-1 | 55 | b1-3 | 30 | — | — | — | — | 8,800 | 1.52 |
| P-16 | PAG-2 | 15 | a1-1 | 55 | b1-4 | 30 | — | — | — | — | 9,200 | 1.51 |
| P-17 | PAG-1 | 15 | a1-1 | 30 | a2-1 | 20 | b1-1 | 35 | — | — | 9,500 | 1.52 |
| P-18 | PAG-3 | 15 | a1-1 | 35 | a3-1 | 15 | b1-2 | 35 | — | — | 9,200 | 1.51 |
| P-19 | PAG-4 | 15 | a1-2 | 30 | a2-1 | 15 | b1-3 | 40 | — | — | 9,100 | 1.52 |
| P-20 | PAG-6 | 10 | a1-1 | 35 | a2-1 | 15 | b1-1 | 30 | b2-1 | 10 | 9,000 | 1.53 |
| P-21 | PAG-7 | 15 | a1-2 | 35 | a3-1 | 15 | b1-2 | 25 | b2-2 | 10 | 9,300 | 1.52 |
| P-22 | PAG-9 | 15 | a1-1 | 50 | b1-1 | 30 | b2-3 | 5 | — | — | 9,500 | 1.54 |
| P-23 | PAG-1 | 5 | a1-1 | 55 | b1-2 | 40 | — | — | — | — | 9,200 | 1.52 |
| P-24 | PAG-2 | 5 | a1-1 | 30 | a1-3 | 25 | b1-2 | 40 | — | — | 9,300 | 1.52 |
| P-25 | PAG-3 | 5 | a1-2 | 30 | a2-1 | 20 | b1-4 | 35 | b2-1 | 10 | 9,200 | 1.51 |
| P-26 | PAG-5 | 5 | a1-1 | 35 | a3-1 | 15 | b1-1 | 30 | b2-2 | 15 | 9,100 | 1.52 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | PAG-A | 15 | a1-1 | 55 | b1-1 | 30 | — | — | — | — | 9,500 | 1.54 |
| CP-2 | PAG-B | 15 | a1-1 | 55 | b1-1 | 30 | — | — | — | — | 9,100 | 1.52 |
| CP-3 | PAG-C | 15 | a1-1 | 55 | b1-1 | 30 | — | — | — | — | 9,300 | 1.53 |
| CP-4 | PAG-D | 15 | a1-1 | 55 | b1-1 | 30 | — | — | — | — | 9,100 | 1.51 |
| CP-5 | PAG-E | 15 | a1-1 | 55 | b1-1 | 30 | — | — | — | — | 9,000 | 1.54 |
| CP-6 | PAG-F | 15 | a1-1 | 55 | b1-1 | 30 | — | — | — | — | 9,500 | 1.54 |
| CP-7 | PAG-B | 15 | a1-2 | 55 | b1-1 | 30 | — | — | — | — | 9,600 | 1.53 |
| CP-8 | PAG-C | 15 | a3-1 | 45 | b1-1 | 40 | — | — | — | — | 9,700 | 1.52 |
| CP-9 | PAG-D | 15 | a1-1 | 55 | b1-3 | 30 | — | — | — | — | 9,500 | 1.53 |
| CP-10 | PAG-E | 15 | a1-1 | 55 | b1-4 | 30 | — | — | — | — | 9,400 | 1.55 |
| CP-11 | PAG-B | 15 | a1-1 | 35 | a3-1 | 15 | b1-2 | 35 | — | — | 9,700 | 1.54 |
| CP-12 | PAG-D | 10 | a1-1 | 35 | a2-1 | 15 | b1-1 | 30 | b2-1 | 10 | 9,500 | 1.53 |
| CP-13 | PAG-C | 15 | a1-2 | 35 | a3-1 | 15 | b1-2 | 25 | b2-2 | 10 | 9,300 | 1.51 |
| CP-14 | PAG-F | 15 | a1-1 | 50 | b1-1 | 30 | b2-3 | 5 | — | — | 9,100 | 1.54 |
| CP-15 | PAG-A | 5 | a1-1 | 55 | b1-2 | 40 | — | — | — | — | 9,300 | 1.52 |
| CP-16 | PAG-D | 5 | a1-2 | 30 | a2-1 | 20 | b1-4 | 35 | b2-1 | 10 | 9,400 | 1.54 |
| CP-17 | a1-1 | 60 | b1-1 | 40 | — | — | — | — | — | — | 5,700 | 1.47 |
| CP-18 | a1-1 | 50 | b1-2 | 30 | b2-1 | 20 | — | — | — | — | 6,100 | 1.48 |

### [3] Preparation of Chemically Amplified Resist Composition

### Examples 3-1 to 3-26 and Comparative Examples 3-1 to 3-18

A chemically amplified resist composition (R-1 to R-26, CR-1 to CR-18) was prepared by dissolving a base polymer (P-1 to P-26) containing an inventive sulfonium salt monomer (PAG-1 to PAG-9), a base polymer (CP-1 to CP-18) containing a comparative sulfonium salt monomer (PAG-A to PAG-F), a photoacid generator (PAG-X to PAG-Z) and a quencher (Q-1 to Q-4) in a solvent containing 0.01 wt % of surfactant A (ONMNOVA Inc.) in accordance with the formulation shown in Tables 3 and 4, and filtering the solution through a Teflon® filter with a pore size of 0.2 μm.

TABLE 3

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 3-1 | R-1 | P-1 (80) | Q-1 (8.0) | — | PGMEA (2250) | EL (2800) | DAA (550) |
| | 3-2 | R-2 | P-2 (80) | Q-1 (8.2) | — | PGMEA (2250) | EL (2800) | DAA (550) |
| | 3-3 | R-3 | P-3 (80) | Q-1 (8.0) | — | PGMEA (2250) | EL (2800) | DAA (550) |
| | 3-4 | R-4 | P-4 (80) | Q-1 (7.8) | — | PGMEA (2250) | EL (2800) | DAA (550) |
| | 3-5 | R-5 | P-5 (80) | Q-1 (8.0) | — | PGMEA (2250) | EL (2800) | DAA (550) |
| | 3-6 | R-6 | P-6 (80) | Q-1 (8.0) | — | PGMEA (2250) | EL (2800) | DAA (550) |
| | 3-7 | R-7 | P-7 (80) | Q-1 (8.2) | — | PGMEA (2250) | EL (2800) | DAA (550) |
| | 3-8 | R-8 | P-8 (80) | Q-1 (8.0) | — | PGMEA (2250) | EL (2800) | DAA (550) |
| | 3-9 | R-9 | P-9 (80) | Q-1 (7.6) | — | PGMEA (2250) | EL (2800) | DAA (550) |
| | 3-10 | R-10 | P-10 (80) | Q-2 (8.2) | — | PGMEA (2250) | EL (2800) | DAA (550) |
| | 3-11 | R-11 | P-11 (80) | Q-3 (8.0) | — | PGMEA (2250) | EL (2800) | DAA (550) |
| | 3-12 | R-12 | P-12 (80) | Q-1 (8.0) | — | PGMEA (2250) | EL (2800) | DAA (550) |
| | 3-13 | R-13 | P-13 (80) | Q-1 (8.2) | — | PGMEA (2250) | EL (2800) | DAA (550) |
| | 3-14 | R-14 | P-14 (80) | Q-1 (8.0) | — | PGMEA (2250) | EL (2800) | DAA (550) |
| | 3-15 | R-15 | P-15 (80) | Q-3 (7.8) | — | PGMEA (2250) | EL (2800) | DAA (550) |
| | 3-16 | R-16 | P-16 (80) | Q-2 (8.0) | — | PGMEA (2250) | EL (2800) | DAA (550) |
| | 3-17 | R-17 | P-17 (80) | Q-1 (8.0) | — | PGMEA (2250) | EL (2800) | DAA (550) |
| | 3-18 | R-18 | P-18 (80) | Q-1 (7.8) | — | PGMEA (2250) | EL (2800) | DAA (550) |
| | 3-19 | R-19 | P-19 (80) | Q-2 (8.0) | — | PGMEA (2250) | EL (2800) | DAA (550) |
| | 3-20 | R-20 | P-20 (80) | Q-3 (7.8) | PAG-Y (15) | PGMEA (2250) | EL (2800) | DAA (550) |
| | 3-21 | R-21 | P-21 (80) | Q-1 (4.0) | — | PGMEA (2250) | EL (2800) | DAA (550) |
| | | | | Q-4 (3.8) | | | | |
| | 3-22 | R-22 | P-22 (80) | Q-1 (8.2) | — | PGMEA (2250) | EL (2800) | DAA (550) |
| | 3-23 | R-23 | P-23 (80) | Q-3 (7.6) | — | PGMEA (2250) | EL (2800) | DAA (550) |
| | 3-24 | R-24 | P-24 (80) | Q-1 (8.0) | PAG-X (10) | PGMEA (2250) | EL (2800) | DAA (550) |
| | 3-25 | R-25 | P-25 (80) | Q-2 (8.2) | PAG-X (8) | PGMEA (2250) | EL (2800) | DAA (550) |
| | | | | | PAG-Z (4) | | | |
| | 3-26 | R-26 | P-26 (80) | Q-3 (8.0) | PAG-Y (10) | PGMEA (2250) | EL (2800) | DAA (550) |
| | | | | | PAG-Z (5) | | | |

TABLE 4

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Comparative | 3-1 | CR-1 | CP-1 (80) | Q-1 (8.0) | — | PGMEA (2250) | EL (2800) | DAA (550) |
| Example | 3-2 | CR-2 | CP-2 (80) | Q-1 (8.2) | — | PGMEA (2250) | EL (2800) | DAA (550) |
| | 3-3 | CR-3 | CP-3 (80) | Q-1 (8.0) | — | PGMEA (2250) | EL (2800) | DAA (550) |
| | 3-4 | CR-4 | CP-4 (80) | Q-1 (7.8) | — | PGMEA (2250) | EL (2800) | DAA (550) |
| | 3-5 | CR-5 | CP-5 (80) | Q-1 (8.0) | — | PGMEA (2250) | EL (2800) | DAA (550) |
| | 3-6 | CR-6 | CP-6 (80) | Q-1 (8.2) | | PGMEA (2250) | EL (2800) | DAA (550) |
| | 3-7 | CR-7 | CP-7 (80) | Q-2 (8.2) | — | PGMEA (2250) | EL (2800) | DAA (550) |
| | 3-8 | CR-8 | CP-8 (80) | Q-1 (8.2) | — | PGMEA (2250) | EL (2800) | DAA (550) |
| | 3-9 | CR-9 | CP-9 (80) | Q-3 (7.8) | — | PGMEA (2250) | EL (2800) | DAA (550) |
| | 3-10 | CR-10 | CP-10 (80) | Q-2 (8.0) | — | PGMEA (2250) | EL (2800) | DAA (550) |
| | 3-11 | CR-11 | CP-11 (80) | Q-1 (8.0) | — | PGMEA (2250) | EL (2800) | DAA (550) |
| | 3-12 | CR-12 | CP-12 (80) | Q-3 (7.8) | PAG-Y (15) | PGMEA (2250) | EL (2800) | DAA (550) |
| | 3-13 | CR-13 | CP-13 (80) | Q-1 (4.0) | — | PGMEA (2250) | EL (2800) | DAA (550) |
| | | | | Q-4 (3.8) | | | | |
| | 3-14 | CR-14 | CP-14 (80) | Q-1 (8.2) | — | PGMEA (2250) | EL (2800) | DAA (550) |
| | 3-15 | CR-15 | CP-15 (80) | Q-3 (7.6) | — | PGMEA (2250) | EL (2800) | DAA (550) |
| | 3-16 | CR-16 | CP-16 (80) | Q-2 (8.2) | PAG-X (8) | PGMEA (2250) | EL (2800) | DAA (550) |
| | | | | | PAG-Z (4) | | | |
| | 3-17 | CR-17 | CP-17 (80) | Q-1 (8.0) | PAG-X (24) | PGMEA (2250) | EL (2800) | DAA (550) |
| | 3-18 | CR-18 | CP-18 (80) | Q-1 (8.0) | PAG-Y (14) | PGMEA (2250) | EL (2800) | DAA (550) |
| | | | | | PAG-Z (7) | | | |

The solvents, photoacid generators PAG-X, PAG-Y, PAG-Z, quenchers Q-1 to Q-4 and surfactant A in Tables 3 and 4 are as identified below.

### Solvent:

PGMEA (propylene glycol monomethyl ether acetate)
EL (ethyl lactate)
DAA (diacetone alcohol)

### Photoacid Generator: PAG-X, PAG-Y, PAG-Z

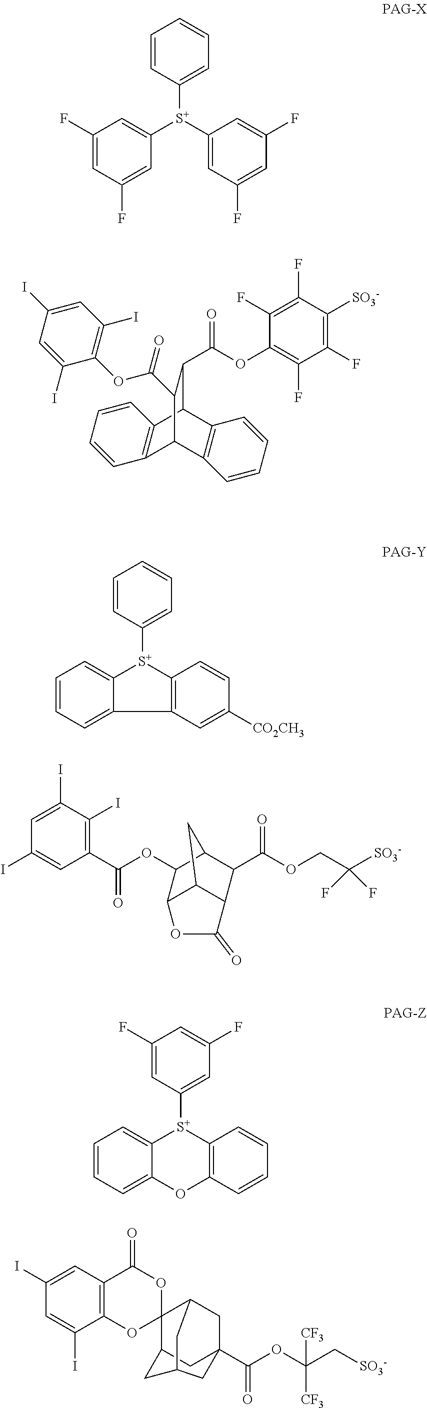

### Quencher: Q-1 to Q-4

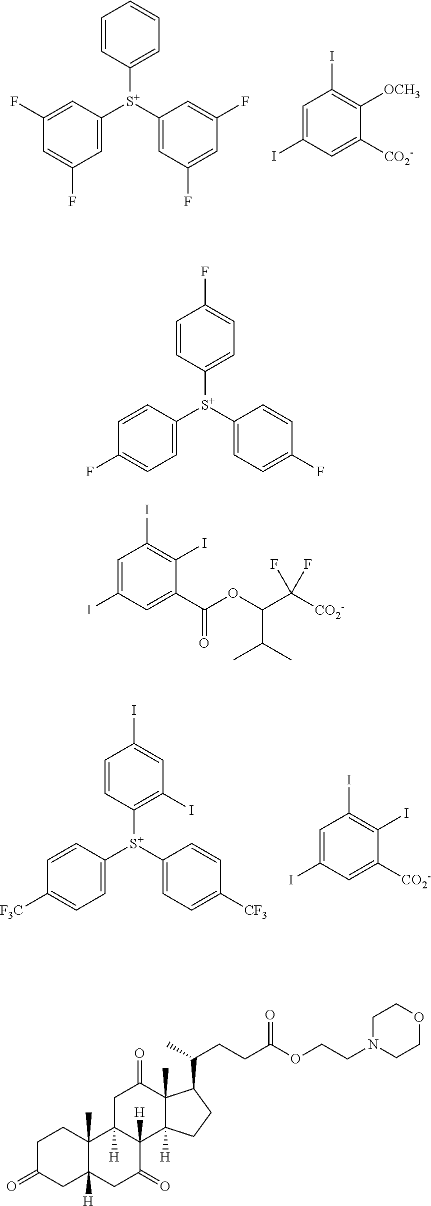

### Surfactant A:

3-methyl-3-(2,2,2-trifluoroethoxymethyl)oxetane/tetrahydrofuran/
2,2-dimethyl-1,3-propanediol copolymer (manufactured by OMNOVA Inc.)

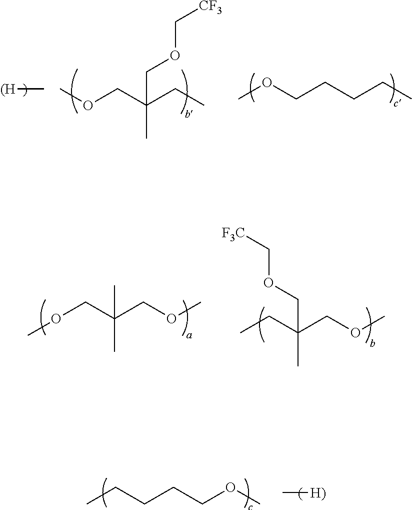

a:(b+b′):(c+c′)=1:4 to 7:0.01 to 1 (molar ratio)
Mw=1,500

### [4] EUV Lithography Test (1)

### Examples 4-1 to 4-26 and Comparative Examples 4-1 to 4-18

Each of the chemically amplified resist compositions (R-1 to R-26, CR-1 to CR-18 in Tables 3 and 4) was spin coated on a silicon substrate having a 20-nm coating of silicon containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 (ASML, NA 0.33, a 0.9/0.6, dipole illumination), the resist film was exposed to EUV through a mask bearing a line-and-space (LS) pattern having a width of 18 nm and a pitch of 36 nm (on-wafer size) while changing the dose at a pitch of 1 mJ/cm² and the focus at a pitch of 0.020 μm. The resist film was baked (PEB) at the temperature shown in Tables 5 and 6 for 60 seconds. This was followed by puddle development in a 2.38 wt % TMAH aqueous solution for 30 seconds, rinsing with a surfactant-containing rinse fluid, and spin drying. A positive LS pattern was obtained.

The LS pattern was observed under CD-SEM (CG6300, Hitachi High-Technologies Corp.) and evaluated for sensitivity, exposure latitude (EL), LWR, depth of focus (DOF), and collapse limit by the following methods. Development defect evaluation was performed on the obtained LS pattern. The results are shown in Tables 5 and 6.

### [Evaluation of Sensitivity]

The optimum dose Eop (mJ/cm²) which provided an LS pattern with a line width of 18 nm and a pitch of 36 nm was determined and reported as sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation. A greater value indicates better performance.

$$EL ( \% ) = ( \vert {E}_{1} - {E}_{2} \vert / Eop )$$

wherein E₁ is an optimum exposure dose which provides a LS pattern with a line width of 16.2 nm and a pitch of 36 nm,

E₂ is an optimum exposure dose which provides a LS pattern with a line width of 19.8 nm and a pitch of 36 nm, and

Eop is an optimum exposure dose which provides a LS pattern with a line width of 18 nm and a pitch of 36 nm. A greater value indicates better performance.

### [Evaluation of LWR]

For the LS pattern formed by exposure at the optimum dose Eop, the line width was measured at 10 longitudinally spaced apart points, from which a 3-fold value (3a) of the standard deviation (a) was determined and reported as LWR. A smaller value of 3a indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

### [Development Defect Evaluation]

The LS pattern of line width 18 nm and pitch 36 nm printed at the optimum dose was counted for defects using a defect inspection system (trade name KLA 2360 by KLA Tencor Corp.). With a pixel size of 0.16 μm and a threshold of 20 set, the number of defects extracted from the difference obtained by overlapping a comparative image and a pixel unit was detected, from which the count of defects per unit area (count/cm²) was computed. On reviewing defects, development defects were classified and extracted from all the defects. The number of development defects per unit area (count/cm²) was computed. A sample is rated “A” for a count of less than 0.5, “B” for a count of 0.5 to less than 1.0, “C” for a count of 1.0 to less than 5.0, and “D” for a count of 5.0 or more. A smaller count indicates better performance.

TABLE 5

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Example | 4-1 | R-1 | 100 | 31 | 17 | 2.1 | 120 | 11.0 | A |
| | 4-2 | R-2 | 100 | 32 | 19 | 2.3 | 120 | 10.9 | A |
| | 4-3 | R-3 | 100 | 31 | 18 | 2.1 | 110 | 10.8 | A |
| | 4-4 | R-4 | 105 | 31 | 18 | 2.3 | 100 | 11.1 | A |
| | 4-5 | R-5 | 100 | 32 | 17 | 2.3 | 120 | 11.2 | A |
| | 4-6 | R-6 | 100 | 34 | 17 | 2.4 | 120 | 11.1 | A |
| | 4-7 | R-7 | 100 | 32 | 18 | 2.2 | 110 | 11.1 | A |
| | 4-8 | R-8 | 95 | 31 | 18 | 2.1 | 110 | 11.4 | A |
| | 4-9 | R-9 | 100 | 32 | 17 | 2.4 | 110 | 10.8 | A |
| | 4-10 | R-10 | 95 | 31 | 19 | 2.2 | 100 | 11.0 | A |
| | 4-11 | R-11 | 100 | 32 | 17 | 2.1 | 110 | 11.1 | A |
| | 4-12 | R-12 | 100 | 31 | 18 | 2.2 | 100 | 10.9 | A |
| | 4-13 | R-13 | 105 | 32 | 17 | 2.3 | 110 | 10.8 | A |
| | 4-14 | R-14 | 100 | 32 | 17 | 2.2 | 110 | 10.8 | A |
| | 4-15 | R-15 | 95 | 31 | 18 | 2.1 | 110 | 10.8 | A |
| | 4-16 | R-16 | 100 | 32 | 17 | 2.2 | 120 | 11.3 | A |
| | 4-17 | R-17 | 100 | 31 | 19 | 2.4 | 120 | 11.2 | A |
| | 4-18 | R-18 | 95 | 31 | 17 | 2.3 | 110 | 11.1 | A |
| | 4-19 | R-19 | 95 | 32 | 18 | 2.2 | 100 | 11.1 | A |
| | 4-20 | R-20 | 100 | 31 | 18 | 2.4 | 100 | 11.3 | A |
| | 4-21 | R-21 | 100 | 32 | 19 | 2.1 | 120 | 10.8 | A |
| | 4-22 | R-22 | 100 | 32 | 17 | 2.2 | 110 | 11.0 | A |
| | 4-23 | R-23 | 95 | 31 | 17 | 2.3 | 110 | 11.2 | A |
| | 4-24 | R-24 | 95 | 33 | 18 | 2.3 | 120 | 11.2 | A |
| | 4-25 | R-25 | 100 | 31 | 17 | 2.2 | 110 | 11.1 | A |
| | 4-26 | R-26 | 100 | 32 | 19 | 2.3 | 110 | 10.9 | A |

TABLE 6

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Comparative | 4-1 | CR-1 | 95 | 43 | 13 | 3.1 | 90 | 12.8 | C |
| Example | 4-2 | CR-2 | 100 | 37 | 12 | 2.8 | 80 | 12.6 | B |
| | 4-3 | CR-3 | 100 | 35 | 13 | 2.7 | 80 | 13.2 | B |
| | 4-4 | CR-4 | 100 | 35 | 14 | 2.8 | 90 | 13.1 | C |
| | 4-5 | CR-5 | 95 | 37 | 14 | 2.6 | 80 | 12.5 | B |
| | 4-6 | CR-6 | 100 | 35 | 12 | 3.0 | 60 | 12.3 | B |
| | 4-7 | CR-7 | 100 | 36 | 13 | 2.8 | 90 | 12.6 | C |
| | 4-8 | CR-8 | 100 | 35 | 14 | 2.7 | 80 | 12.3 | B |
| | 4-9 | CR-9 | 95 | 37 | 15 | 2.7 | 90 | 13.3 | B |
| | 4-10 | CR-10 | 100 | 35 | 14 | 2.6 | 80 | 12.9 | C |
| | 4-11 | CR-11 | 100 | 36 | 13 | 2.7 | 80 | 12.6 | B |
| | 4-12 | CR-12 | 100 | 36 | 13 | 2.8 | 80 | 13.0 | C |
| | 4-13 | CR-13 | 95 | 35 | 15 | 2.6 | 90 | 12.5 | B |
| | 4-14 | CR-14 | 105 | 36 | 13 | 3.1 | 50 | 12.2 | B |
| | 4-15 | CR-15 | 100 | 42 | 13 | 3.0 | 70 | 12.4 | C |
| | 4-16 | CR-16 | 95 | 38 | 14 | 2.8 | 80 | 13.3 | C |
| | 4-17 | CR-17 | 95 | 41 | 12 | 3.6 | 60 | 12.7 | C |
| | 4-18 | CR-18 | 100 | 40 | 12 | 3.4 | 60 | 12.2 | B |

As seen from Tables 5 and 6, chemically amplified resist compositions comprising polymers comprising repeat units derived from sulfonium salt monomers within the scope of the invention exhibit a high sensitivity and improved lithography properties including EL, LWR and DOF. Small values of collapse limit show that small-size patterns have resistance to collapse. Restraint of development defects is also confirmed. This demonstrates that chemically amplified resist compositions are suitable as materials for EUV lithography.

### [5] EUV Lithography Test (2)

### Examples 5-1 to 5-26 and Comparative Examples 5-1 to 5-18

Each of the chemically amplified resist compositions (R-1 to R-26, CR-1 to CR-18 in Tables 3 and 4) was spin coated on a silicon substrate having a 20-nm coating of silicon containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, a 0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 7 and 8 for 60 seconds. This was followed by development in a 2.38 wt % TMAH aqueous solution for 30 seconds. Hole patterns with a size of 23 nm were obtained.

The hole pattern was observed under CD-SEM (CG6300, Hitachi High-Technologies Corp.). The exposure dose Eop that provides a hole pattern having a size of 23 nm was determined and reported as sensitivity. The size of 50 holes at that dose was measured, from which a 3-fold value (3σ) of the standard deviation (σ) was computed and reported as CDU. The results are shown in Tables 7 and 8.

TABLE 7

| | | | | Optimum | |
|---|---|---|---|---|---|
| | | Resist | PEB temp. | exposure dose | CDU |
| | | composition | (°C) | (mJ/cm²) | (nm) |
| Example | 5-1 | R-1 | 90 | 23 | 2.2 |
| | 5-2 | R-2 | 95 | 22 | 2.3 |
| | 5-3 | R-3 | 95 | 24 | 2.1 |
| | 5-4 | R-4 | 90 | 23 | 2.2 |
| | 5-5 | R-5 | 95 | 22 | 2.4 |
| | 5-6 | R-6 | 95 | 24 | 2.2 |
| | 5-7 | R-7 | 95 | 22 | 2.3 |
| | 5-8 | R-8 | 95 | 23 | 2.1 |
| | 5-9 | R-9 | 95 | 22 | 2.3 |
| | 5-10 | R-10 | 95 | 24 | 2.2 |
| | 5-11 | R-11 | 95 | 22 | 2.2 |
| | 5-12 | R-12 | 90 | 23 | 2.4 |
| | 5-13 | R-13 | 90 | 23 | 2.3 |
| | 5-14 | R-14 | 95 | 24 | 2.2 |
| | 5-15 | R-15 | 90 | 22 | 2.2 |
| | 5-16 | R-16 | 90 | 23 | 2.4 |
| | 5-17 | R-17 | 90 | 22 | 2.2 |
| | 5-18 | R-18 | 95 | 23 | 2.2 |
| | 5-19 | R-19 | 95 | 22 | 2.3 |
| | 5-20 | R-20 | 90 | 24 | 2.4 |
| | 5-21 | R-21 | 95 | 22 | 2.2 |
| | 5-22 | R-22 | 95 | 22 | 2.1 |
| | 5-23 | R-23 | 90 | 24 | 2.2 |
| | 5-24 | R-24 | 95 | 22 | 2.3 |
| | 5-25 | R-25 | 95 | 24 | 2.2 |
| | 5-26 | R-26 | 95 | 22 | 2.3 |

TABLE 8

| | | | | Optimum | |
|---|---|---|---|---|---|
| | | Resist | PEB temp. | exposure dose | CDU |
| | | composition | (°C) | (mJ/cm²) | (nm) |
| Comparative | 5-1 | CR-1 | 95 | 33 | 3.2 |
| Example | 5-2 | CR-2 | 95 | 26 | 2.8 |
| | 5-3 | CR-3 | 95 | 27 | 2.8 |
| | 5-4 | CR-4 | 90 | 26 | 2.7 |
| | 5-5 | CR-5 | 90 | 26 | 2.8 |
| | 5-6 | CR-6 | 95 | 25 | 3.4 |
| | 5-7 | CR-7 | 90 | 29 | 2.7 |
| | 5-8 | CR-8 | 90 | 26 | 2.6 |
| | 5-9 | CR-9 | 90 | 25 | 2.7 |
| | 5-10 | CR-10 | 95 | 27 | 2.6 |
| | 5-11 | CR-11 | 95 | 27 | 2.6 |
| | 5-12 | CR-12 | 95 | 28 | 2.8 |
| | 5-13 | CR-13 | 85 | 26 | 2.7 |
| | 5-14 | CR-14 | 95 | 26 | 3.2 |
| | 5-15 | CR-15 | 95 | 34 | 3.5 |
| | 5-16 | CR-16 | 90 | 28 | 2.8 |
| | 5-17 | CR-17 | 95 | 33 | 3.4 |
| | 5-18 | CR-18 | 95 | 32 | 3.3 |

It is demonstrated in Tables 7 and 8 that chemically amplified resist compositions comprising polymers comprising repeat units derived from sulfonium salt monomers within the scope of the invention exhibit a high sensitivity and improved CDU.

Japanese Patent Application No. 2025-062615 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt monomer having the formula (A): 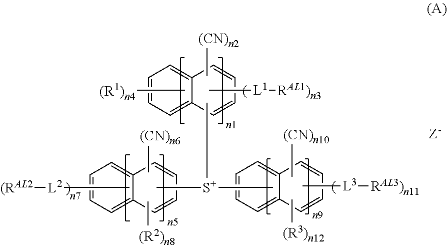
wherein n1 is 0 or 1, n2 is 0, 1 or 2, n3 is 0, 1 or 2, n4 is 0, 1 or 2, provided that 0≤n2+n3+n4≤5 when n1=0, and 0≤n2+n3+n4≤7 when n1=1; n5 is 0 or 1, n6 is 0, 1 or 2, n7 is 0, 1 or 2, n8 is 0, 1 or 2, provided that 0≤n6+n7+n8≤5 when n5=0, and 0≤n6+n7+n8≤7 when n5=1; n9 is 0 or 1, n10 is 0, 1 or 2, n11 is 0, 1 or 2, n12 is 0, 1 or 2, provided that 0≤n10+n11+n12≤5 when n9=0, and 0≤n10+n11+n12≤7 when n9=1, and 1≤n2+n6+n10≤6 and 1≤n3+n7+n11≤6,
R¹, R² and R³ are each independently halogen, nitro, hydroxy, carboxy, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom; when n4=2, two R¹ may be identical or different and may bond together to form a ring with the carbon atoms to which they are attached; when n8=2, two R² may be identical or different and may bond together to form a ring with the carbon atoms to which they are attached; when n12=2, two R³ may be identical or different and may bond together to form a ring with the carbon atoms to which they are attached,
L¹, L² and L³ are each independently an ether bond, ester bond or carbonate bond, two L¹ may be identical or different when n3=2, two L² may be identical or different when n7=2, two L³ may be identical or different when n11=2,
R^{AL}, R^{AL2} and R^{AL3} are each independently an acid labile group, two R^{AL1} may be identical or different when n3=2, two R^{AL2} may be identical or different when n7=2, two R^{AL3} may be identical or different when n11=2,
two of three aromatic rings bonded to S⁺ may bond together to form a ring with the sulfur atom to which they are attached, and
Z⁻ is a fluoroalkanesulfonate anion having a polymerizable group.

2. The sulfonium salt monomer of claim 1, having the formula (A1): 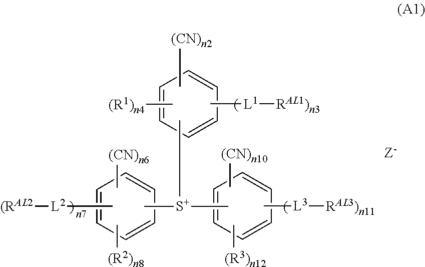
wherein n2 to n4, n6 to n8, n10 to n12, R¹ to R³, L¹ to L³, R^{AL1} to R^{AL3}, and Z⁻ are as defined above.

3. The sulfonium salt monomer of claim 1, wherein the acid labile group has the formula (AL-1) or (AL-2): 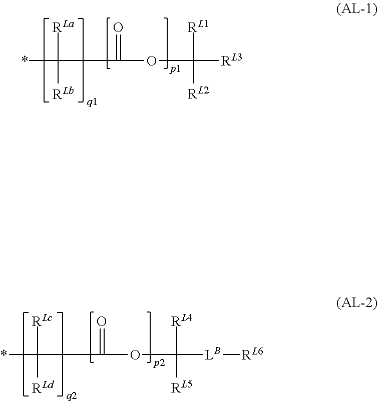
wherein p1 and p2 are each independently 0 or 1, q1 and q2 are each independently 0, 1, 2, 3 or 4,
R^{L1} and R^{L2} are each independently a C₁-C₁₂ hydrocarbyl group, R^{L3} is hydrogen or a C₁-C₁₂ hydrocarbyl group, some constituent —CH₂— in the hydrocarbyl groups R^{L1}, R^{L2} and R^{L3} may be replaced by —O— or —S—, and when hydrocarbyl group contains an aromatic ring, some or all of the hydrogen atoms on the aromatic ring may be substituted by halogen, cyano, nitro, a C₁-C₄ alkyl group which may contain a halogen atom or C₁-C₄ alkoxy group which may contain a halogen atom, R^{L1} and R^{L2} may bond together to form a ring with the carbon atom to which they are attached, and some constituent —CH₂— in the ring may be replaced by —O— or —S—, provided that when R^{L3} is hydrogen, R^{L1} and R^{L2} bond together to form an alicyclic ring containing a multiple bond together with the carbon atom to which they are bonded, some of the hydrogen atoms on the alicyclic ring may be replaced by halogen,
R^{L4} and R^{L5} are each independently hydrogen or a C₁-C₁₀ hydrocarbyl group, R^{L6} is a C₁-C₂₀ hydrocarbyl group in which some constituent —CH₂— may be replaced by —O— or —S—, R^{L5} and R^{L6} may bond together to form a C₃-C₂₀ heterocyclic group with the carbon atom and L⁴ to which they are attached, and some constituent —CH₂— in the heterocyclic group may be replaced by —O— or —S—,
L⁴ is —O— or —S—,
R^{La} to R^{Ld} are each independently hydrogen or a C₁-C₂₀ hydrocarbyl group which may contain a heteroatom, and
* designates a point of attachment to L¹, L² and L³.

4. The sulfonium salt monomer of claim 1, wherein Z⁻ is an anion having the formula (Z1): 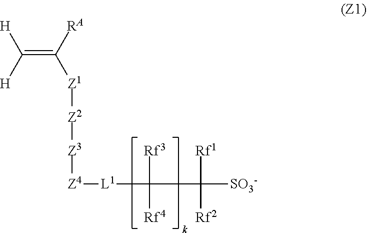
wherein k is 0, 1, 2 or 3,
R^{A} is each independently hydrogen, fluorine, methyl or trifluoromethyl,
Z¹ is each independently a single bond, optionally substituted phenylene group, naphthylene group or *—C(═O)—O—Z¹¹—, Z¹¹ is a C₁-C₁₀ aliphatic hydrocarbylene group which may contain halogen, hydroxy, ether bond, ester bond or lactone ring, a phenylene group or naphthylene group,
Z² is a single bond, ether bond, ester bond, amide bond, sulfonate ester bond, sulfonamide bond, carbonate bond or carbamate bond,
Z³ is each independently a single bond, ***—Z³¹—C(═O)—O—, ***—C(═O)—N(H)—Z³¹— or ***—O—Z³¹—, Z³¹ is a C₁-C₂₀ hydrocarbylene group which may contain a heteroatom,
Z⁴ is each independently a single bond, ****—Z⁴¹—C(═O)—O—, ****—C(═O)—N(H)—Z⁴¹— or ****—O—Z⁴¹—, Z⁴¹ is a C₁-C₂₀ hydrocarbylene group which may contain a heteroatom,
* designates a point of attachment to the carbon atom in the backbone, *** designates a point of attachment to Z², **** designates a point of attachment to Z³,
L⁵ is a single bond, ether bond, ester bond, carbonyl group, sulfonate ester bond, sulfonamide bond, carbonate bond or carbamate bond,
Rf¹ and Rf² are each independently fluorine or a C₁-C₆ fluorinated saturated hydrocarbyl group, and
Rf³ and Rf⁴ are each independently hydrogen, fluorine or a C₁-C₆ fluorinated saturated hydrocarbyl group.

5. The sulfonium salt monomer of claim 1, wherein Z⁻ is an anion having the formula (Z2): 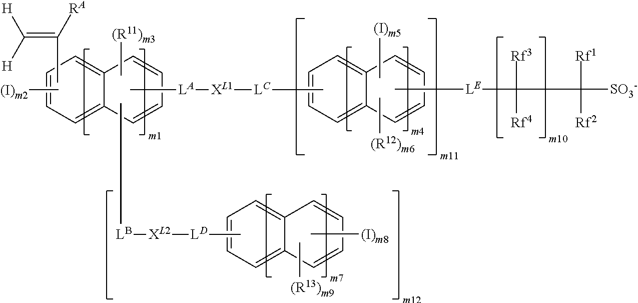
wherein m1 is 0 or 1, m2 is 0, 1, 2, 3 or 4, m3 is 0, 1, 2 or 3, m4 is 0 or 1, m5 is 0, 1, 2, 3 or 4, m6 is 0, 1, 2 or 3, m7 is 0 or 1, m8 is 1, 2, 3 or 4, m9 is 0, 1, 2 or 3, m10 is 0, 1, 2, 3 or 4, m11 is 0 or 1, m12 is 0 or 1, provided that 0≤m2+m3+m12≤4 when m1=0 and 0≤m2+m3+m12≤6 when m1=1; 0≤m5+m6≤4 when m4=0 and 0≤m5+m6≤6 when m4=1, 0≤m8+m9≤5 when m7=0 and 0≤m8+m9≤7 when m7=1, and 1≤m2+m5+m8≤4,
R^{A} is hydrogen, fluorine, methyl or trifluoromethyl,
R¹¹, R¹² and R¹³ are each independently halogen exclusive of iodine, nitro, cyano, hydroxy, carboxy, a C₁-C₂₀ hydrocarbyl group which may contain a heteroatom, C₁-C₂₀ hydrocarbyloxy group which may contain a heteroatom, C₁-C₂₀ hydrocarbylthio group which may contain a heteroatom or C₂-C₂₀ hydrocarbyloxycarbonyl group which may contain a heteroatom; when m3 is 2 or 3, a plurality of R¹¹ may be identical or different and two R¹¹ may bond together to form a ring with the carbon atoms to which they are attached; when m6 is 2 or 3, a plurality of R¹² may be identical or different and two R¹² may bond together to form a ring with the carbon atoms to which they are attached; when m9 is 2 or 3, a plurality of R¹³ may be identical or different and two R¹³ may bond together to form a ring with the carbon atoms to which they are attached;
L^{A}, L^{B}, L^{C}, L^{D} and L^{E} are each independently a single bond, ether bond, ester bond, sulfonate ester bond, amide bond, sulfonamide bond, carbonate bond or carbamate bond,
X^{L1} and X^{L2} are each independently a single bond or a C₁-C₄₀ hydrocarbylene group which may contain a heteroatom,
Rf¹ and Rf² are each independently a fluorine atom or a C₁-C₆ fluorinated saturated hydrocarbyl group,
Rf³ and Rf⁴ are each independently hydrogen, fluorine or a C₁-C₆ fluorinated saturated hydrocarbyl group,
provided that m11 and m12 are not simultaneously 0, and L^{A}, L^{B}, L^{C}, L^{D}, X^{L1} and X^{L2} are not simultaneously single bonds.

6. A monomeric photoacid generator in the form of the sulfonium salt monomer of claim 1.

7. A polymer comprising repeat units derived from the monomeric photoacid generator of claim 6.

8. The polymer of claim 7, further comprising repeat units of at least one type selected from repeat units having the formula (a1), repeat units having the formula (a2), and repeat units having the formula (a3): 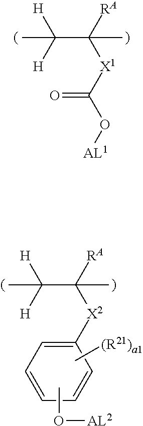
wherein R^{A} is each independently hydrogen, fluorine, methyl or trifluoromethyl,
X¹ is a single bond, phenylene group, naphthylene group, *—C(═O)—O—X¹¹— or *—C(═O)—N(H)—X¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O— or *—C(═O)—N(H)—,
* designates a point of attachment to the carbon atom in the backbone,
R²¹ is halogen, cyano, hydroxy, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom; when a1 is 2, 3 or 4, a plurality of R²¹ may be identical or different,
AL¹ and AL² are each independently an acid labile group, and
a1 is 0, 1, 2, 3 or 4, 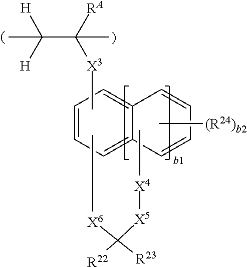
wherein b1 is 0 or 1, b2 is 0, 1, 2 or 3 when b1=0 and 0, 1, 2, 3, 4 or 5 when b1=1,
R^{A} is hydrogen, fluorine, methyl or trifluoromethyl,
X³ is a single bond, *—C(═O)—O— or *—C(═O)—N(H)—, * designates a point of attachment to the carbon atom in the backbone,
X⁴ is a single bond, a C₁-C₄ aliphatic hydrocarbylene group, carbonyl group, sulfonyl group or a group obtained by combining the foregoing,
X⁵ and X⁶ are each independently oxygen or sulfur, X⁴ and X⁶ are attached to adjacent carbon atoms on the aromatic ring,
R²² and R²³ are each independently hydrogen or a C₁-C₂₀ hydrocarbyl group which may contain a heteroatom, R²² and R²³ may bond together to form a ring with the carbon atom to which they are attached,
R²⁴ is halogen, hydroxy, cyano, nitro, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{24A})(R^{24B}), R^{24A} and R^{24B} are each independently hydrogen or a C₁-C₆ hydrocarbyl group; when b2 is 2 or more, a plurality of R²⁴ may be identical or different and may bond together to form a ring with the carbon atom on the aromatic ring to which they are attached.

9. The polymer of claim 7, further comprising repeat units of at least one type selected from repeat units having the formula (b1) and repeat units having the formula (b2): 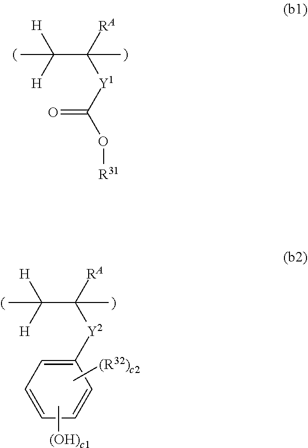
wherein R^{A} is each independently hydrogen, fluorine, methyl or trifluoromethyl,
Y¹ is a single bond, phenylene group, naphthylene group, *—C(═O)—O—Y¹¹— or *—C(═O)—N(H)—Y¹¹—, the phenylene and naphthylene groups may be substituted with hydroxy, nitro, cyano, a C₁-C₁₀ saturated hydrocarbyl moiety which may contain fluorine, C₁-C₁₀ saturated hydrocarbyloxy moiety which may contain fluorine, or halogen, Y¹¹ is a C₁-C₁₀ saturated hydrocarbylene group, phenylene group or naphthylene group, the saturated hydrocarbylene group may contain hydroxy, ether bond, ester bond or lactone ring,
Y² is a single bond or *—C(═O)—O—, * designates a point of attachment to the carbon atom in the backbone,
R³¹ is hydrogen or a C₁-C₂₀ group which contains at least one structure selected from hydroxy exclusive of phenolic hydroxy, cyano, nitro, carbonyl, carboxy, ether bond, ester bond, sulfonate ester bond, carbonate bond, lactone ring, sultone ring, and carboxylic anhydride (—C(═O)—O—C(═O)—),
R³² is halogen, carboxy, nitro, cyano, pentafluorosulfanyl, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom; when c2 is 2, 3 or 4, a plurality of R³² may be identical or different,
c1 is 1, 2, 3 or 4, c2 is 0, 1, 2, 3 or 4, provided that 1≤c1+c2≤5.

10. A chemically amplified resist composition comprising (A) a base polymer containing the polymer of claim 7.

11. The chemically amplified resist composition of claim 10, further comprising (B) an organic solvent.

12. The chemically amplified resist composition of claim 10, further comprising (C) a quencher.

13. The chemically amplified resist composition of claim 10, further comprising (D) a photoacid generator.

14. The chemically amplified resist composition of claim 10, further comprising (E) a surfactant.

15. A pattern forming process comprising the steps of applying the chemically amplified resist composition of claim 10 onto a substrate to form a resist film thereon, exposing the resist film to high-energy radiation, and developing the exposed resist film in a developer.

16. The pattern forming process of claim 15, wherein the high-energy radiation is KrF excimer laser, ArF excimer laser, EB or EUV of wavelength 3 to 15 nm.
